(19) United States
(12) Patent Application Publication
(10) Pub. No.: US 2025/0154171 A1
(43) Pub. Date: May 15, 2025

(54) RAS INHIBITORS

(71) Applicant: Revolution Medicines, Inc., Redwood City, CA (US)

(72) Inventors: John E. KNOX, Emerald Hills, CA (US); Elena S. KOLTUN, Foster City, CA (US); Jinyu LIU, Union City, CA (US); Yang LIU, Foster City, CA (US); Benjamin D. MADEJ, San Leandro, CA (US); Dylan E. PARSONS, San Carlos, CA (US); Aidan TOMLINSON, San Francisco, CA (US); Jingwei YIN, Redwood City, CA (US); Georgios ALACHOUZOS, Redwood City, CA (US); G.Leslie BURNETT, Redwood City, CA (US); Neil CHAN, GRONINGEN (NL); James CREGG, Belmont, CA (US); Anne V. EDWARDS, San Mateo, CA (US); Adrian L. GILL, Atherton, CA (US)

(21) Appl. No.: 18/913,242

(22) Filed: Oct. 11, 2024

Related U.S. Application Data
(60) Provisional application No. 63/638,805, filed on Apr. 25, 2024.
(60) Provisional application No. 63/611,600, filed on Dec. 18, 2023.
(60) Provisional application No. 63/543,841, filed on Oct. 12, 2023.

(51) Int. Cl. C07D 519/00; A61K 31/5025; A61K 31/504; A61K 31/506; A61K 31/5377; A61K 45/06; C07D 513/22

(52) U.S. Cl. CPC C07D 519/00; A61K 31/5025; A61K 31/504; A61K 31/506; A61K 31/5377; A61K 45/06; C07D 513/22

(57) ABSTRACT

The disclosure features macrocyclic compounds, and pharmaceutical compositions and protein complexes thereof, capable of inhibiting Ras proteins, and their uses in the treatment of cancers.

### BACKGROUND

The vast majority of small molecule drugs act by binding a functionally important pocket on a target protein, thereby modulating the activity of that protein. For example, cholesterol-lowering drugs known as statins bind the enzyme active site of HMG-COA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18:674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras (ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by the various Ras mutations.

### SUMMARY

Provided herein are Ras inhibitors and compounds useful for studying Ras inhibition. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF and PI3K, which are required for propagating the oncogenic signal.

As such, in some embodiments, the disclosure features a compound, or pharmaceutically acceptable salt or a stereoisomer thereof, of structural Formula I:

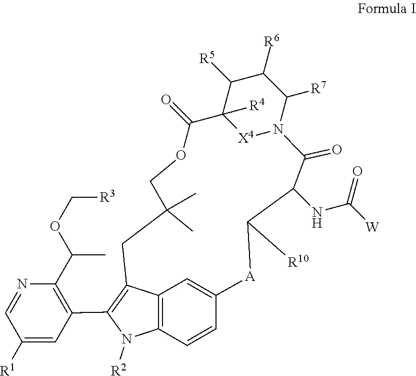

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is —OR¹¹ or —NR¹²R¹³; and
R¹¹, R¹², and R¹³ are each, independently, optionally substituted C₁-C₆ alkyl, optionally substituted C₁-C₆ heteroalkyl, or R¹² and R¹³ combine to form an optionally substituted 3- to 10-membered heterocycloalkyl.

In some embodiments, the disclosure features a compound, or a pharmaceutically acceptable salt or a stereoisomer thereof, of structural Formula I′:

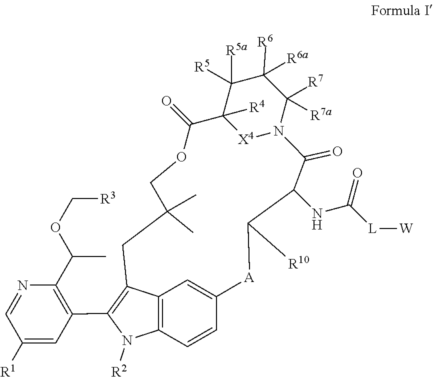

wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
L is a bond or —CH(R²⁰)—N(R²¹)—C(O)—, wherein R²⁰ and R²¹ are each independently an optionally substituted C₁₋₆ alkyl; or R²⁰ and R²¹ taken together with the atoms to which they are attached form 4-10 membered heterocycloalkyl;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R^{5a}, R^{6a} and R^{7a} are each independently hydrogen, halo or C₁-C₆ alkyl;
or R⁵ and R⁶ taken together with the atoms to which they are attached form optionally substituted 3- to 7-membered cycloalkyl or optionally substituted 3- to 7-membered heterocycloalkyl;
or R⁶ and R⁷ taken together with the atoms to which they are attached form optionally substituted 3- to 7-membered cycloalkyl or optionally substituted 3- to 7-membered heterocycloalkyl;
or R⁵ and R^{5a} taken together with the atom to which they are attached form optionally substituted 3- to 7-membered cycloalkyl or an optionally substituted 3- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 3- to 7-membered heterocycloalkyl;
or R⁵ and R⁶ taken together form a bond;
R¹⁰ is H, —OR¹¹ or —NR¹²R¹³; R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl;
and
when R¹⁰ is H, then ring A is optionally substituted 2,4-oxazol-diyl or optionally substituted 2,5-oxazol-diyl and R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt or a stereoisomer thereof.

In some embodiments, the disclosure features a compound, or a pharmaceutically acceptable salt or a stereoisomer thereof, of structural Formula I′-a:

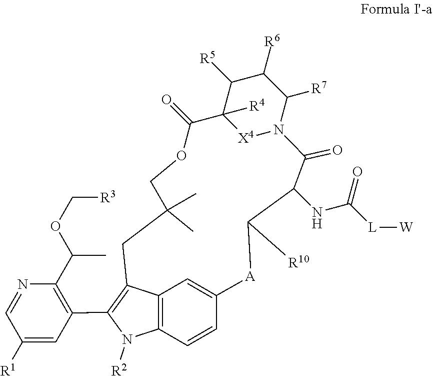

wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
L is a bond or —CH(R²⁰)—N(R²¹)—C(O)—, wherein R²⁰ and R²¹ are each independently an optionally substituted C₁₋₆ alkyl; or R²⁰ and R²¹ taken together with the atoms to which they are attached form 4-10 membered heterocycloalkyl;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl;
and
when R¹⁰ is H, then ring A is optionally substituted 2,4-oxazol-diyl or optionally substituted 2,5-oxazol-diyl and R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt or a stereoisomer thereof.

In some embodiments, one or more compounds of WO 2022/060836 is excluded. In some embodiments, one or more compounds of Table 3 may be excluded from any embodiment herein.

Also provided are pharmaceutical compositions comprising a compound of Formula I or Formula I′ or a pharmaceutically acceptable salt or stereoisomer thereof, and a pharmaceutically acceptable excipient. Also provided are pharmaceutical compositions comprising a compound of Table 1, or a pharmaceutically acceptable salt or stereoisomer thereof, and a pharmaceutically acceptable excipient.

Also provided are pharmaceutical compositions comprising a compound of Table 2, or a pharmaceutically acceptable salt or stereoisomer thereof, and a pharmaceutically acceptable excipient.

Also provided is a method of treating cancer in a subject in need thereof, the method comprising administering to the subject a therapeutically effective amount of a compound of the present invention, or a pharmaceutically acceptable salt or a stereoisomer thereof.

In some embodiments, a method is provided of treating a Ras protein-related disorder in a subject in need thereof, the method comprising administering to the subject a therapeutically effective amount of a compound of the present invention, or a pharmaceutically acceptable salt or a stereoisomer thereof.

Further provided is a method of inhibiting a Ras protein in a cell, the method comprising contacting the cell with an effective amount of a compound of the present invention, or a pharmaceutically acceptable salt or a stereoisomer thereof.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I or Formula I′ and subformulae thereof, for example, a compound of Table 1 or Table 2, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H—, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

Non-limiting examples of moieties that may contain one or more deuterium substitutions in compounds of the present invention, where any position “R” may be deuterium (D), include

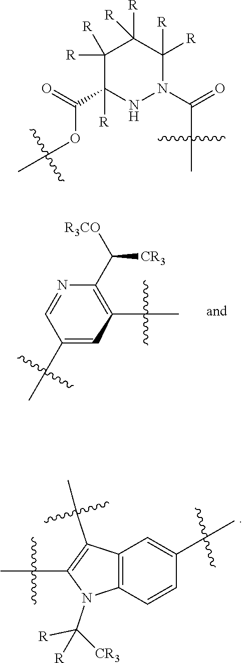

Additional examples include moieties such as

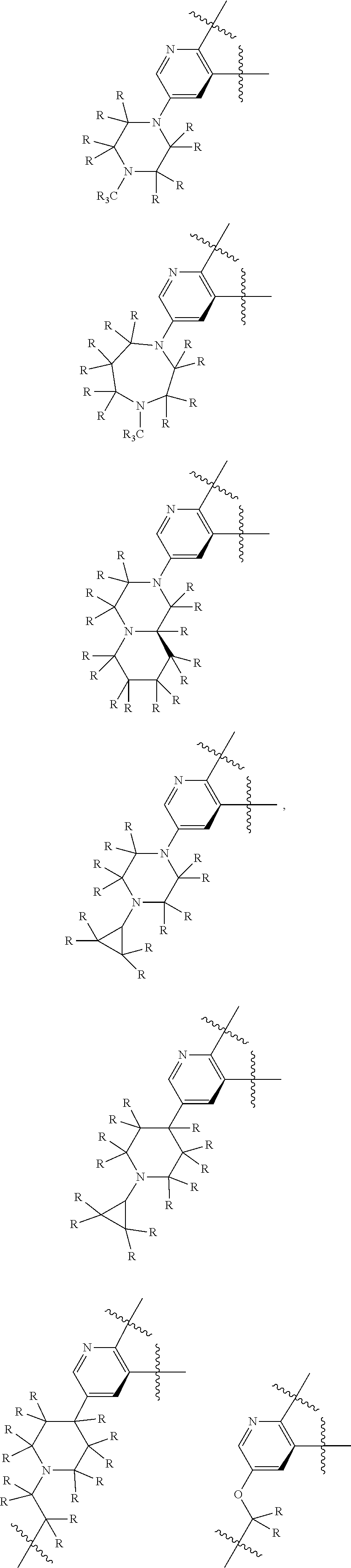

and deuteration of similar moieties, e.g., in compounds of Formula I and subformulae thereof). Moreover, deuteration of available positions in any A moiety of compounds of the Formulas described herein is also contemplated, such as

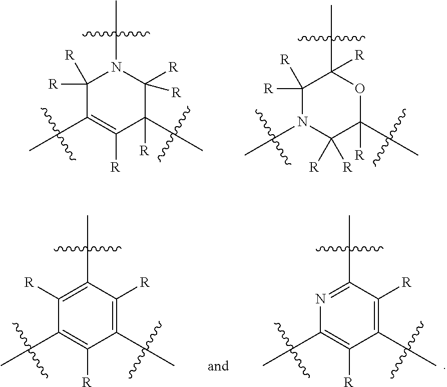

Further, deuterium substitution may also take place in compounds of the present invention at the linker position, such as

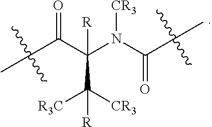

Further, deuterium substitution may also take place in compounds of the present invention at the linker position, such as

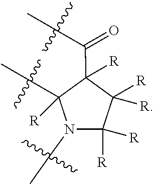

Further, deuterium substitution may also take place in compounds of the present invention at the linker position, such as

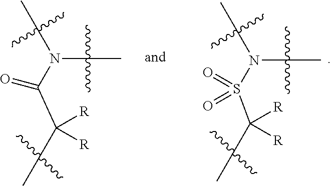

In a further embodiment, silylation substitution is also contemplated, such as in the linker as follows:

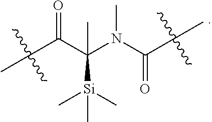 [SMILES: *C(=O)N(C)[C@@](C)(C(*)=O)[Si](C)(C)C]

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; oxo; halogen; —(CH₂)₀₋₄R^{o}; —(CH₂)₀₋₄OR^{o}; —O(CH₂)₀₋₄R^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; 4- to 8-membered saturated or unsaturated heterocycloalkyl (e.g., pyridyl); 3- to 8-membered saturated or unsaturated cycloalkyl (e.g., cyclopropyl, cyclobutyl, or cyclopentyl); —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄—C(O)—N(R^{o})₂; —(CH₂)₀₋₄—C(O)—N(R^{o})—S(O)₂—R^{o}; —C(NCN)NR^{o}₂; —(CH₂)₀₋₄C(O)SR^{o}; —(CH₂)₀₋₄C(O)oSiR^{o}₃; —(CH₂)₀₋₄OC(O)R^{o}; —OC(O)(CH₂)₀₋₄SR^{o}; —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NOR^{o})NR^{o}₂; —C(NH)NR^{o}₂; —P(O)₂R^{o}; —P(O)R^{o}₂; —P(O)(OR^{o})₂; —OP(O)R^{o}₂; —OP(O)(OR^{o})₂; —OP(O)(OR^{o})R^{o}; —SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ alkyl, C₂₋₆ alkenyl, C₂₋₆ alkynyl, —C₁₋₆ aliphatic, C₃₋₁₀ cycloalkyl, C₆₋₁₀ aryl, 5- to 14-membered heteroaryl having 1-4 ring heteroatoms selected from O, N and S, 4- to 14-membered heterocycloalkyl having 1-4 ring heteroatoms selected from O, N, S, SO and SO₂, wherein the nitrogen is optionally oxidized, C₃₋₁₀ cycloalkyl-C₁₋₆ alkyl-, C₆₋₁₀ aryl-C₁₋₆ alkyl-, (5-14-membered heteroaryl)-C₁₋₆ alkyl, (4-14 membered heterocycloalkyl)-C₁₋₆ alkyl-, —CH₂Ph, —O(CH₂)₀₋₁ Ph, —CH₂-(5- to 6-membered heteroaryl ring), —CH₂-(5- to 10-membered heteroaryl ring), 3- to 15-membered saturated, partially unsaturated, or aryl ring having 0-4 heteroatoms independently selected from nitrogen, oxygen, sulfur, SO or SO₂, wherein the nitrogen is optionally oxidized; or a 3- to 6-membered saturated, partially unsaturated, or aryl ring having 0-4 heteroatoms independently selected from nitrogen, oxygen, or sulfur, wherein the nitrogen is optionally oxidized; or, notwithstanding the definition above, two independent occurrences of R^{o}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

R^{o} (or the ring formed by taking two independent occurrences of R^{o} together with their intervening atoms), may be substituted with 1, 2, 3, 4, or 5 substituents independently selected from halogen, oxo, CN, N₃, C₁₋₆ alkyl, C₂₋₆ alkenyl, C₂₋₆ alkynyl, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}2, —NO₂, —SiR^{●}₃, —OSiR^{●}₃, —C(O)SR^{●}, —(C₁₋₄ straight or branched alkylene)C(O)OR^{●}, —SSR^{●}, —(CH₂)₀₋₄N(R′)₂; —(CH₂)₀₋₄N(R′)C(O)R′; —N(R′)C(S)R′; —(CH₂)₀₋₄N(R′)C(O)NR′₂; —N(R′)C(S)NR′₂; —(CH₂)₀₋₄N(R′)C(O)OR′; —N(R′)N(R′)C(O)R′; —N(R′)N(R′)C(O)NR′₂; —N(R′)N(R′)C(O)OR′; —(CH₂)₀₋₄C(O)R′; —C(S)R′; —(CH₂)₀₋₄C(O)OR′; —(CH₂)₀₋₄—C(O)—N(R′)₂; —(CH₂)₀₋₄—C(O)—N(R^{o})—S(O)₂—R′; —C(NC N)NR′₂; —(CH₂)₀₋₄C(O)SR′; —(CH₂)₀₋₄C(O)OSiR′₃; —(CH₂)₀₋₄OC(O)R′; —OC(O)(CH₂)₀₋₄SR′; —SC(S)SR′; —(CH₂)₀₋₄SC(O)R′; —(CH₂)₀₋₄C(O)NR′₂; —C(S)NR′₂; —C(S)SR^{o}; —(CH₂)₀₋₄OC(O)NR′₂; —C(O)N(OR′)R′; —C(O)C(O)R′; —C(O)CH₂C(O)R′; —C(NOR′)R′; —(CH₂)₀₋₄SSR′; —(CH₂)₀₋₄S(O)₂R′; —(CH₂)₀₋₄S(O)₂OR′; —(CH₂)₀₋₄OS(O)₂R′; —S(O)₂NR′₂; —(CH₂)₀₋₄S(O)R′; —N(R′)S(O)₂NR′₂; —N(R′)S(O)₂R′; —N(OR′)R′; —C(NOR′)NR′₂; —C(NH)NR′₂; —P(O)₂R′; —P(O)R′₂; —P(O)(OR′)₂; —OP(O)R′₂; —OP(O)(OR′)₂; —OP(O)(OR′)R′; wherein R^{●} and R′ are each optionally substituted with 1, 2, 3, 4 or 5 R″, or where preceded by “halo” is substituted only with one or more halogens, and are each independently selected from C₁₋₆ alkyl, C₂₋₆ alkenyl, C₂₋₆ alkynyl, C₃₋₁₀ cycloalkyl, C₆₋₁₀ aryl, 5- to 14-membered heteroaryl having 1-4 ring heteroatoms selected from O, N and S, 4- to 14-membered heterocycloalkyl having 1-4 ring heteroatoms selected from O, N, S, SO and SO₂, wherein the nitrogen is optionally oxidized, C₃₋₁₀ cycloalkyl-C₁₋₆ alkyl-, C₆₋₁₀ aryl-C₁₋₆ alkyl-, (5-14-membered heteroaryl)-C₁₋₆ alkyl, (4-14 membered heterocycloalkyl)-C₁₋₆ alkyl-C₁₋₄ aliphatic, —CH₂Ph, —O(CH₂)₀₋₁Ph, a 3- to 8-membered saturated, partially unsaturated, or aryl ring having 0-4 heteroatoms independently selected from nitrogen, oxygen, or sulfur, or a 5- to 6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

R″ is C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-14 membered heterocycloalkyl)-C₁₋₄ alkyl-, each of which is optionally substituted with 1, 2 or 3 independently selected R′″ substituents.

Each R′″ is independently selected from 5- to 10-membered heteroaryl, (5- to 10-membered heteroaryl)-C₁₋₄ alkyl, 4- to 14 membered heterocycloalkyl, (4- to 14 membered heterocycloalkyl)-C₁₋₄ alkyl, phenyl, phenyl-C₁₋₄ alkyl, C₃₋₁₀ cycloalkyl, C₃₋₁₀ cycloalkyl-C₁₋₄ alkyl, C₁₋₆ alkyl, C₁₋₆ alkoxy, C₁₋₆ haloalkyl, C₁₋₆ haloalkoxy, C₂₋₆ alkenyl, C₂₋₆ alkynyl, halogen, CN, OR^{a}, SR^{a}, NHOR^{a}, C(O)R^{a}, C(O)NR^{a}R^{a}, C(O)OR^{a}, C(O)NR^{a}S(O)₂R^{a}, OC(O)R^{a}, OC(O)NR^{a}R^{a}, NHR^{a}, NR^{a}R^{a}, NR^{a}C(O)R^{a}, NR^{a}C(═NR^{a})R^{a}, NR^{a}C(O)OR^{a}, NR^{a}C(O)NR^{a}R^{a}, C(═NR^{a})R^{a}, C(═NOH)R^{a}, C(═NOH)NR^{a}, C(═NCN)NR^{a}R^{a}, NR^{a}C(═NCN)NR^{a}R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, S(O)₂NR^{a}C(O)R^{a}, —P(O)R^{a}R^{a}, —P(O)(OR^{a})(OR^{a}), —B(OH)₂, —B(OR^{a})₂ and S(O)₂NR^{a}R^{a}, wherein the 5- to 10-membered heteroaryl, (5- to 6-membered heteroaryl)-C₁₋₄ alkyl, 4- to 14 membered heterocycloalkyl, (4- to 14 membered heterocycloalkyl)-C₁₋₄ alkyl, phenyl, phenyl-C₁₋₄ alkyl, C₃₋₁₀ cycloalkyl, C₃₋₁₀ cycloalkyl-C₁₋₄ alkyl, C₁₋₆ alkyl, C₁₋₆ alkoxy, C₂₋₆ alkenyl, and C₂₋₆ alkynyl of R′″ are each optionally substituted with 1, 2, or 3 independently selected R^{b} substituents.

Each R^{a} is independently selected from H, C₁₋₆ alkyl, C₁₋₆ haloalkyl, C₂₋₆ alkenyl, C₂₋₆ alkynyl, phenyl, C₃₋₁₄ cycloalkyl, 5-10 membered heteroaryl, 4-14 membered heterocycloalkyl, phenyl-C₁₋₄ alkyl, C₃₋₁₀ -cycloalkyl-C₁₋₄ alkyl, (5-10 membered heteroaryl)-C₁₋₄ alkyl, and (4-14 membered heterocycloalkyl)-C₁₋₄ alkyl, each of which is optionally substituted with 1, 2, or 3 independently selected R^{d} substituents.

Each R^{b} is independently selected from oxo, CN, halogen, NH₂, OH, COOH, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₁₄ cycloalkyl, 5-10 membered heteroaryl, 4-14 membered heterocycloalkyl, phenyl-C₁₋₄ alkyl, C₃₋₁₀ cycloalkyl-C₁₋₄ alkyl, (5-10 membered heteroaryl)-C₁₋₄ alkyl, and (4-14 membered heterocycloalkyl)-C₁₋₄ alkyl, NHOR^{c}, OR^{c}, SR^{c}, C(O)R^{c}, C(O)NR^{c}R^{c}, C(O)OR^{c}, C(O)NR^{c} S(O)₂R^{c}, OC(O)R^{c}, OC(O)NR^{c}R^{c}, C(═NOH)R^{c}, C(═NOH)NR^{c}, C(═NCN)NR^{c}R^{c}, NR^{c}C(═NCN)NR^{c}R^{c}, C(═NR^{c})NR^{c}R^{c}, NR^{c}C(═NR^{c})NR^{c}R^{c}, NHR^{c}, NR^{c}R^{c}, NR^{c}C(O)R^{c}, NR^{c}C(═NR^{c})R^{c}, NR^{c}C(O)OR^{c}, NR^{c}C(O)NR^{c}R^{c}, NRCS(O)R^{c}, NRCS(O)₂R^{c}, NR^{c} S(O)₂NR^{c}R^{c}, S(O)R^{c}, S(O)NR^{c}R^{c}, S(O)₂R^{c}, S(O)₂NR^{c}C(O)R^{c}, —P(O)R^{c}R^{c}, —P(O)(OR^{c})(OR^{c}), —B(OH)₂, —B(OR^{C})₂ and S(O)₂NR^{c}R^{c}; Each R^{c} is independently selected from H, C₁₋₆ alkyl, C₁₋₄ haloalkyl, C₂₋₆ alkenyl, C₂₋₆ alkynyl, phenyl, C₃₋₁₀ cycloalkyl, 5-10 membered heteroaryl, 4-10 membered heterocycloalkyl, phenyl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl;
wherein each R^{d} is independently selected from H, C₁₋₆ alkyl, C₁₋₄ haloalkyl, C₂₋₆ alkenyl, C₂₋₆ alkynyl, phenyl, C₃₋₁₀ cycloalkyl, 5-10 membered heteroaryl, 4-10 membered heterocycloalkyl, phenyl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl;
wherein two R′ substituents together with the nitrogen atom to which they are attached can form a 4-, 5-, 6-, 6-, 8-, 9- or 10-membered heterocycloalkyl group optionally substituted with 1, 2, or 3 independently selected R^{e} substituents;
wherein any two R^{a} substituents together with the nitrogen atom to which they are attached can form a 4-, 5-, 6-, 6-, 8-, 9- or 10-membered heterocycloalkyl group optionally substituted with 1, 2, or 3 independently selected R^{e} substituents;
wherein two R^{c} substituents together with the nitrogen atom to which they are attached can form a 4-, 5-, 6-, 6-, 8-, 9- or 10-membered heterocycloalkyl group optionally substituted with 1, 2, or 3 independently selected R^{e} substituents;
wherein each R^{e} is independently selected from C₁₋₆ alkyl, C₁₋₆ alkoxy, C₁₋₆ haloalkyl, C₁₋₆ haloalkoxy, C₂₋₆ alkenyl, C₂₋₆ alkynyl, CN, OH, —COOH, —COOR^{f}, NH₂, —NHR^{f}, —N(R^{f})₂, —C(O)NHR^{f}, —SO₂R^{f}, —NHC(O)R^{f}, —C(O)R^{f}, NR^{′f}S(O)₂R^{′f}, NR^{′f}S(O)₂NR^{′f}R^{′f}, S(O)₂NR^{′f}C(O)R^{′f}, S(O)₂NR^{′f}R^{f}, phenyl, C₃₋₆ cycloalkyl, 4-6 membered heterocycloalkyl, or 5-6 membered heteroaryl;
wherein each R^{f} is independently selected from H, C₁₋₆ alkyl, phenyl, C₃₋₆ cycloalkyl, 4-6 membered heterocycloalkyl, and 5-6 membered heteroaryl; or wherein two R^{f} substituents together with the nitrogen atom to which they are attached can form a 4-, 5-, 6-, 6-, 8-, 9- or 10-membered heterocycloalkyl group optionally substituted with 1, 2, or 3 independently selected R^{g} substituents.

In some embodiments, R^{g} can be halogen, CN, OH, —COOH, —NH₂, —NH—C₁₋₆ alkyl, —N(C₁₋₆ alkyl)₂, C₁₋₆ alkyl, C₁₋₆ alkoxy, C₁₋₆ haloalkyl, C₁₋₆ haloalkyl, C₁₋₆ haloalkoxy, —C(O)NH₂, —C(O)NH—C₁₋₆ alkyl, phenyl, C₃₋₆ cycloalkyl, 4-6 membered heterocycloalkyl, or 5-6 membered heteroaryl.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, a 3- to 8-membered saturated, partially unsaturated, or aryl ring having 0-4 heteroatoms independently selected from nitrogen, oxygen, or sulfur, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁ Ph, a 3- to 8-membered saturated, partially unsaturated, or aryl ring having 0-4 heteroatoms independently selected from nitrogen, oxygen, or sulfur, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted -oPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on an aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁ Ph, a 3- to 8-membered saturated, partially unsaturated, or aryl ring having 0-4 heteroatoms independently selected from nitrogen, oxygen, or sulfur, or a 5- to 6-membered saturated, partially unsaturated, or aryl ring having 0-4 heteroatoms independently selected from nitrogen, oxygen, or sulfur. Suitable divalent substituents on a saturated carbon atom of Rt include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

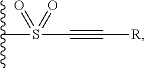

wherein R is any chemically feasible substituent described herein.

The term “amino,” as used herein, represents-N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

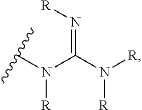

wherein each R is, independently, any any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic, or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heterocycloalkyl,” as used herein, represents a monovalent monocyclic, bicyclic, or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more-OH moieties.

The term “isomer,” as used herein, means any tautomer, stereoisomer, atropisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., one portion of a macrocycle) to a second moiety (e.g., a second portion of the same macrocycle). In some embodiments, the linker results in a compound capable of achieving an IC50 of 1 uM or less in the Ras-RAF disruption assay protocol provided here:
The purpose of this biochemical assay is to measure the ability of test compounds to facilitate ternary complex formation between a nucleotide-loaded Ras isoform and cyclophilin A; the resulting ternary complex disrupts binding to a BRAFRBD construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless cyclophilin A, His6-K-Ras-GMPPNP (or other Ras variant), and GST-BRAFRBD are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras: RAF complex are identified as those eliciting a decrease in the TR-FRET ratio relative to DMSO control wells.
   This assay may be used to assess selectivity as well. In some embodiments, a compound of the present invention is selective for one or more particular Ras mutants over other Ras mutants or wild-type compared to what is known in the art. For example, a compound of the present invention may be more selective for KRAS^{G12V} versus wild-type KRAS.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

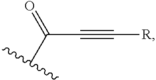

wherein R is any any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that non-covalent interactions of a compound of the present invention with Ras and the chaperone protein (e.g., cyclophilin A) contribute to the inhibition of Ras activity. For example, van der Waals, hydrophobic, hydrophilic and hydrogen bond interactions, and combinations thereof, may contribute to the ability of the compounds of the present invention to form complexes and act as Ras inhibitors.

A variety of Ras proteins may be inhibited by a compound of the present invention (e.g., K-Ras, N-Ras, H-Ras, and mutants thereof at positions 12, 13 and 61, such as G12C, G12D, G12V, G12S, G12R, G13C, G13D, Q61H, Q61K, Q61R and Q61L, and others described herein, or a combination thereof). In some embodiments, a compound of the present invention inhibits a G12C, G12D, G12R or G12S mutant of RAS, or a combination thereof. In some embodiments, a compound of the present invention interacts selectively with K-RasG12V versus wildtype and versus other mutants.

Accordingly, provided herein is a compound having the structure of Formula I′:

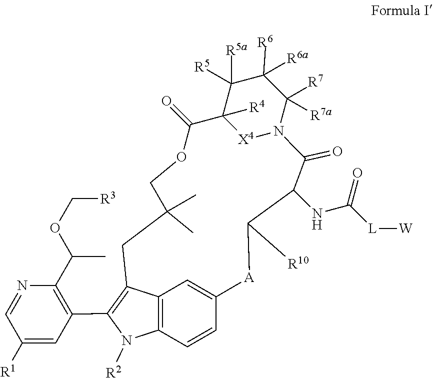

wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
L is a bond or —CH(R²⁰)—N(R²¹)—C(O)—, wherein R²⁰ and R²¹ are each independently an optionally substituted C₁₋₆ alkyl; or R²⁰ and R²¹ taken together with the atoms to which they are attached form 4-10 membered heterocycloalkyl; W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R^{5a}, R^{6a} and R^{7a} are each independently hydrogen, halo or C₁-C₆ alkyl;
or R⁵ and R⁶ taken together with the atoms to which they are attached form optionally substituted 3- to 7-membered cycloalkyl or optionally substituted 3- to 7-membered heterocycloalkyl;
or R⁶ and R⁷ taken together with the atoms to which they are attached form optionally substituted 3- to 7-membered cycloalkyl or optionally substituted 3- to 7-membered heterocycloalkyl;
or R⁵ and R^{5a} taken together with the atom to which they are attached form optionally substituted 3- to 7-membered cycloalkyl or an optionally substituted 3- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 3- to 7-membered heterocycloalkyl;
or R⁵ and R⁶ taken together form a bond;
R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl;
and
when R¹⁰ is H, then ring A is optionally substituted 2,4-oxazol-diyl or optionally substituted 2,5-oxazol-diyl and R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt or a stereoisomer thereof.

In some embodiments, each of R⁴, R⁵, R⁶, and R⁷ are hydrogen. In some embodiments, R⁴ and R⁶ are hydrogen and R⁵ and R⁷ combine together with the atoms to which they are attached, to form an optionally substituted four-membered cycloalkyl. In some embodiments, R⁵ and R⁷ are hydrogen and R⁴ and R⁶ combine, together with the atoms to which they are attached, to form an optionally substituted four-membered cycloalkyl.

In some embodiments, R^{5a}, R^{6a} and R^{7a} are each independently hydrogen, halo or C₁-C₆ alkyl.

In some embodiments, R⁵ and R⁶ taken together with the atoms to which they are attached form optionally substituted 4-membered cycloalkyl;

In some embodiments, R⁶ and R⁷ taken together with the atoms to which they are attached form optionally substituted 4-membered cycloalkyl.

In some embodiments, R⁵ and R^{5a} are taken together with the atom to which they are attached form an optionally substituted 4-membered cycloalkyl;

In some embodiments, R⁶ and R^{6a} taken together with the atom to which they are attached form optionally substituted 4-membered cycloalkyl;

In some embodiments, R⁷ and R^{7a} are taken together with the atom to which they are attached form an optionally 4-membered cycloalkyl.

In some embodiments, provided herein is a compound of Formula I′:

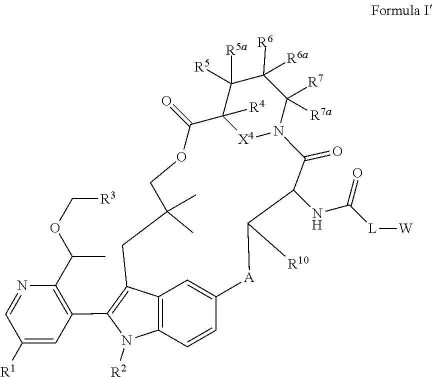

wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
L is a bond or —CH(R²⁰)—N(R²¹)—C(O)—, wherein R²⁰ and R²¹ are each independently an optionally substituted C₁₋₆ alkyl; or R²⁰ and R²¹ taken together with the atoms to which they are attached form 4-10 membered heterocycloalkyl;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R^{5a}, R^{6a} and R^{7a} are each independently hydrogen, halo or C₁-C₆ alkyl;
or R⁵ and R⁶ taken together with the atoms to which they are attached form optionally substituted 3- to 7-membered cycloalkyl or optionally substituted 3- to 7-membered heterocycloalkyl;
or R⁶ and R⁷ taken together with the atoms to which they are attached form optionally substituted 3- to 7-membered cycloalkyl or optionally substituted 3- to 7-membered heterocycloalkyl;
or R⁵ and R^{5a} taken together with the atom to which they are attached form optionally substituted 3- to 7-membered cycloalkyl or an optionally substituted 3- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 3- to 7-membered heterocycloalkyl;
or R⁵ and R⁶ taken together form a bond; R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 5- to 10-membered heteroaryl, optionally substituted 4-14 membered heterocycloalkyl, optionally substituted C₃-C₁₀ cycloalkyl or R¹² and R¹³ combine to form an optionally substituted 3- to 10-membered heterocycloalkyl;
and
when R¹⁰ is H, then ring A is optionally substituted 2,4-oxazol-diyl or optionally substituted 2,5-oxazol-diyl and R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt thereof.

In some embodiments, the moiety:

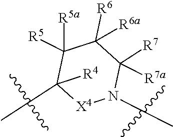

in Formula I′ is selected from:

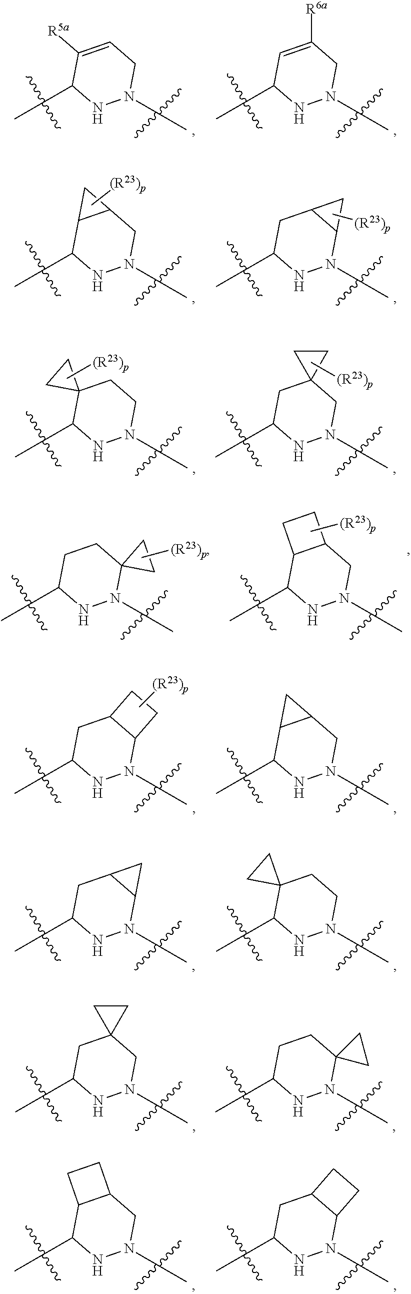

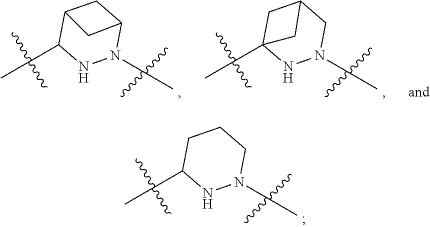

wherein:
R^{5a} is H, halogen or C₁₋₆ alkyl;
R^{6a} is H, halogen or C₁₋₆ alkyl;
each R²³ is independently halogen or C₁₋₆ alkyl; and
each p is independently an integer of 0, 1 or 2.

In some embodiments, provided herein is a compound of Formula I′-a:

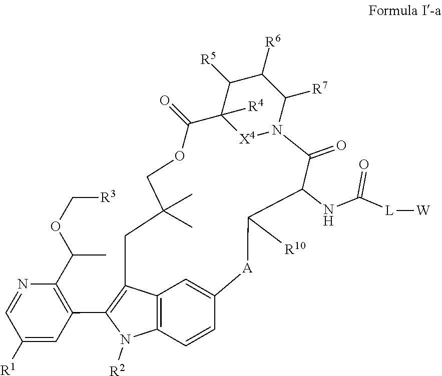

wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
L is a bond or —CH(R²⁰)—N(R²¹)—C(O)—, wherein R²⁰ and R²¹ are each independently an optionally substituted C₁₋₆ alkyl; or R²⁰ and R²¹ taken together with the atoms to which they are attached form 4-10 membered heterocycloalkyl;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl;
and
when R¹⁰ is H, then ring A is optionally substituted 2,4-oxazol-diyl or optionally substituted 2,5-oxazol-diyl and R² is hydrogen, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt or a stereoisomer thereof.

Also provided herein is a compound having the structure of Formula I:

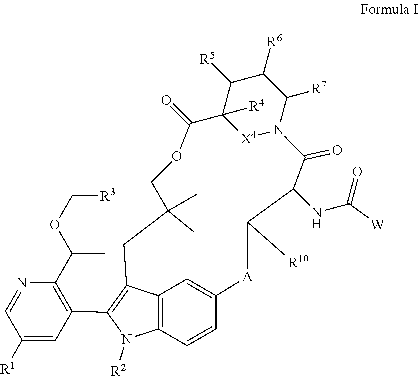

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;

each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is —OR¹¹ or —NR¹²R¹³; and
R¹¹, R¹², and R¹³ are each, independently, optionally substituted C₁-C₆ alkyl, optionally substituted C₁-C₆ heteroalkyl, or R¹² and R¹³ combine to form an optionally substituted 3- to 10-membered heterocycloalkyl
or a pharmaceutically acceptable salt or a stereoisomer thereof.

In some embodiments, the compound has the structure of Formula I

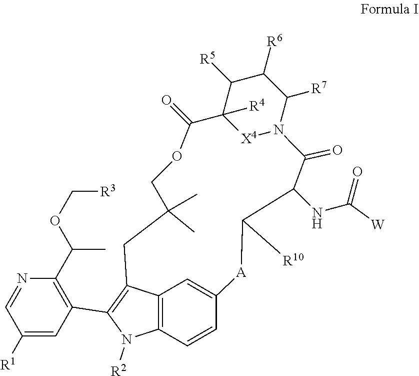

wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl; and
when R¹⁰ is H, then ring A is optionally substituted 2,4-oxazol-diyl or optionally substituted 2,5-oxazol-diyl and R² is hydrogen, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
   or a pharmaceutically acceptable salt or a stereoisomer thereof.

In some embodiments, ring A is optionally substituted 2,4-oxazol-diyl or optionally substituted 2,5-oxazol-diyl; R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R¹⁰ is H.

In some embodiments, ring A is optionally substituted 2,4-oxazol-diyl or optionally substituted 2,5-oxazol-diyl;
R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and
R¹⁰ is H.

In some embodiments, the compound has the structure of Formula I′-b

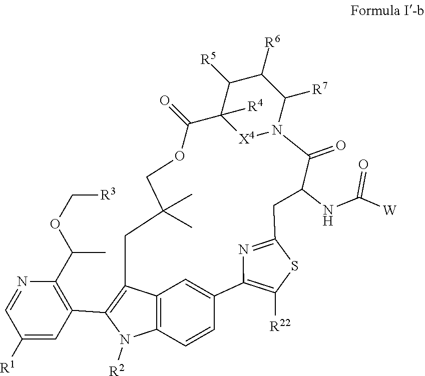

wherein R²² is H, CN, OH, optionally substituted C₁₋₆ alkyl, optionally substituted C₁₋₆ alkoxy, optionally substituted C₃₆ cycloalkyl or optionally substituted 4- to 6-membered heterocycloalkyl; and
R² is substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, or optionally substituted 5 or 6-membered heteroaryl.

In some embodiments of compounds disclosed herein, R² is optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, or optionally substituted 5 or 6-membered heteroaryl.

In some embodiments, provided herein is a compound having the structure of Formula I:

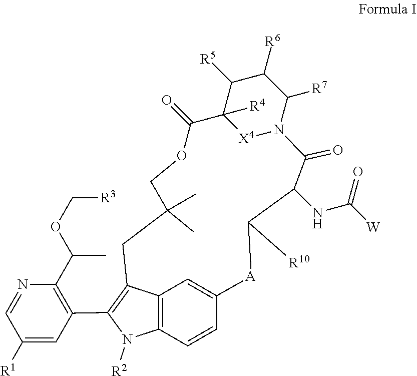

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is —OR¹¹ or —NR¹²R¹³; and
R¹¹, R¹², and R¹³ are each, independently, optionally substituted C₁-C₆ alkyl, optionally substituted C₁-C₆ heteroalkyl, or R¹² and R¹³ combine to form an optionally substituted 3- to 10-membered heterocycloalkyl;
or a pharmaceutically acceptable salt thereof.

In some embodiments, the compound is not a compound of Table 3.

In some embodiments, X⁴ is NH. In some embodiments, X⁴ is CH₂.

In some embodiments, a compound of the disclosure has the structure of Formula Ia, or a pharmaceutically acceptable salt or a stereoisomer thereof:

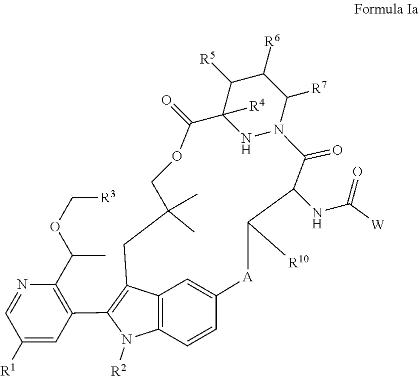

In some embodiments, a compound of the disclosure has the structure of Formula Ib, or a pharmaceutically acceptable salt or a stereoisomer thereof:

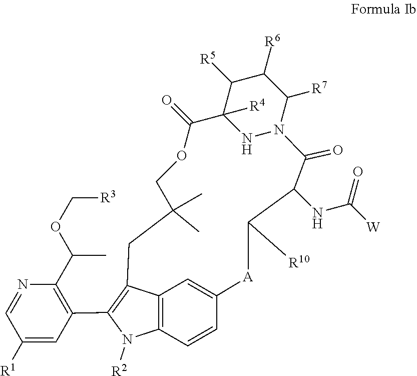

In some embodiments of compounds disclosed herein, each of R⁴, R⁵, R⁶, and R⁷ are hydrogen.

In some embodiments, a compound of the disclosure has the structure of Formula Ic, or a pharmaceutically acceptable salt or a stereoisomer thereof:

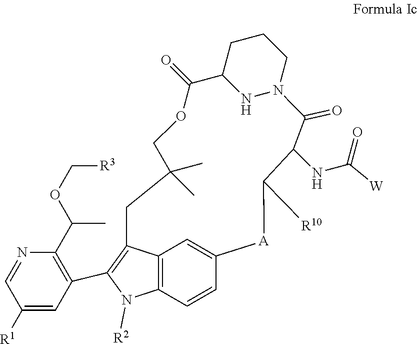

In some embodiments, a compound of the disclosure has the structure of Formula Id, or a pharmaceutically acceptable salt or a stereoisomer thereof:

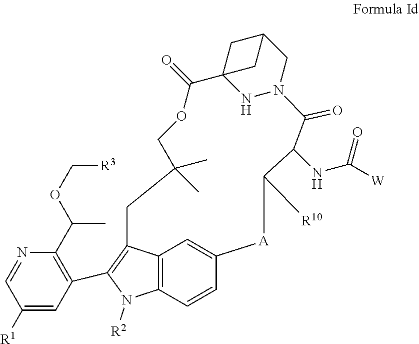

In some embodiments, a compound of the disclosure has the structure of Formula Ie, or a pharmaceutically acceptable salt or a stereoisomer thereof:

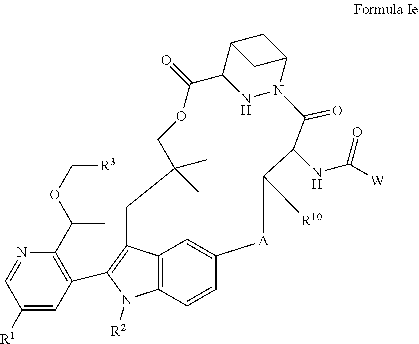

In some embodiments of compounds disclosed herein, A is optionally substituted 5 to 6-membered heteroarylene. In some embodiments of compounds disclosed herein, A is oxazole-diyl or thiazole-diyl.

In some embodiments, a compound of the disclosure has the structure of Formula Ic-1, or a pharmaceutically acceptable salt or a stereoisomer thereof:

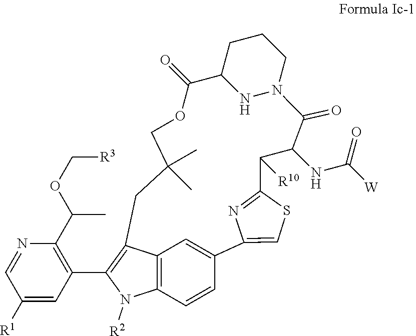

In some embodiments, a compound of the disclosure has the structure of Formula Id-1, or a pharmaceutically acceptable salt or a stereoisomer thereof:

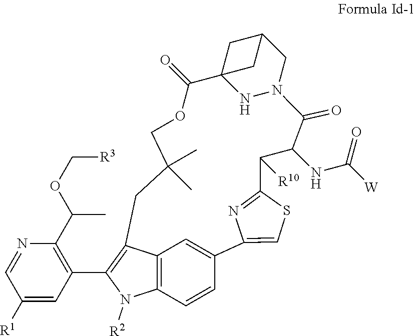

In some embodiments, a compound of the disclosure has the structure of Formula Ie-1, or a pharmaceutically acceptable salt or a stereoisomer thereof:

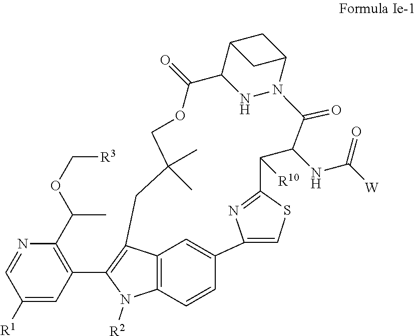

In some embodiments, a compound of the disclosure has the structure of Formula Ic-2, or a pharmaceutically acceptable salt or a stereoisomer thereof:

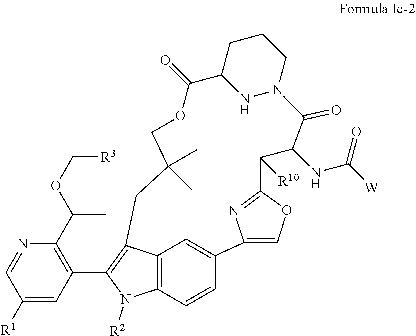

In some embodiments, a compound of the disclosure has the structure of Formula Id-2, or a pharmaceutically acceptable salt or a stereoisomer thereof:

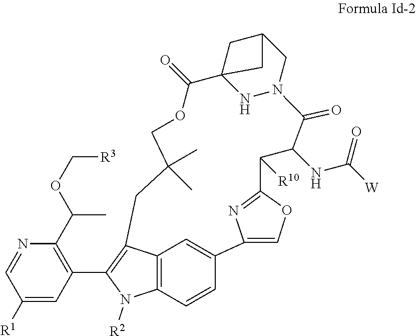

In some embodiments, a compound of the disclosure has the structure of Formula Ie-2, or a pharmaceutically acceptable salt or a stereoisomer thereof:

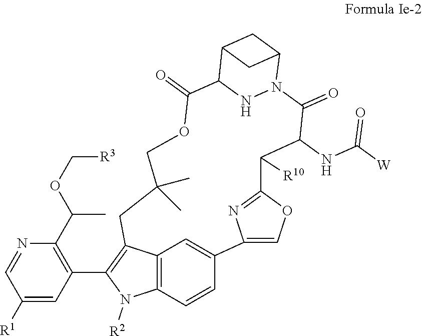

In some embodiments of compounds disclosed herein, R³ is hydrogen. In some embodiments, R² and R³ combine to form an optionally substituted 8- to 14-membered heterocycloalkyl. In some embodiments, R² is optionally substituted C₁-C₆ heteroalkyl or optionally substituted 3 to 7-membered heterocycloalkyl. In some embodiments, R² is optionally substituted C₁-C₆ heteroalkyl. In some embodiments, R² is optionally substituted 3 to 7-membered heterocycloalkyl.

In some embodiments, a compound of the disclosure has the structure of Formula Ic-3, or a pharmaceutically acceptable salt or a stereoisomer thereof:

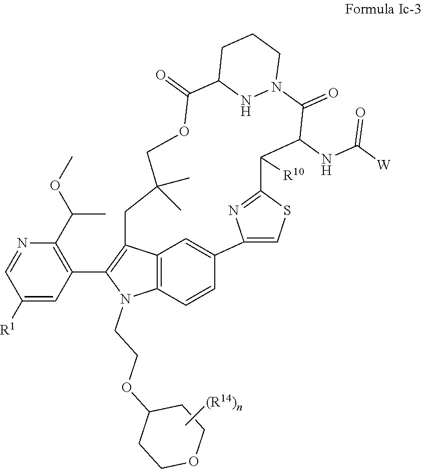

wherein each R¹⁴ is independently C₁-C₃ alkyl; and n is 0, 1, 2, or 3.

In some embodiments, a compound of the disclosure has the structure of Formula Id-3, or a pharmaceutically acceptable salt or a stereoisomer thereof:

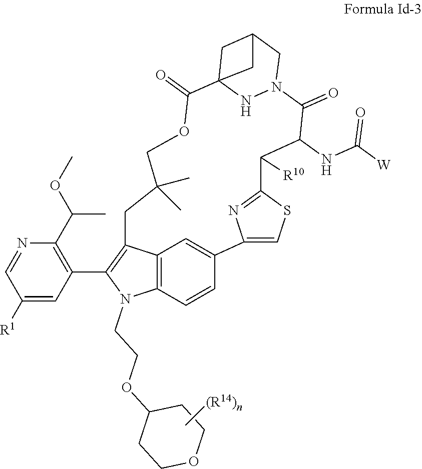

wherein each R¹⁴ is independently C₁-C₃ alkyl; and
n is 0, 1, 2, or 3.

In some embodiments, a compound of the disclosure has the structure of Formula Ie-3, or a pharmaceutically acceptable salt or a stereoisomer thereof:

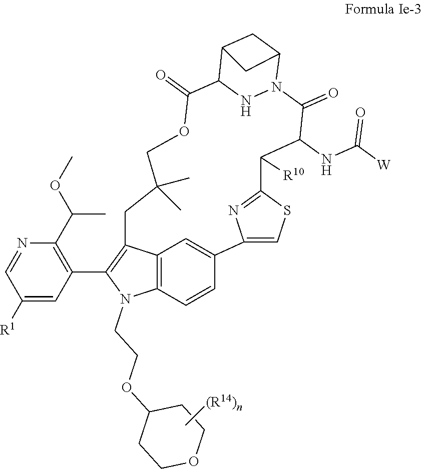

wherein each R¹⁴ is independently C₁-C₃ alkyl; and
n is 0, 1, 2, or 3.

In some embodiments, a compound of the disclosure has the structure of Formula Ic-4, or a pharmaceutically acceptable salt or a stereoisomer thereof:

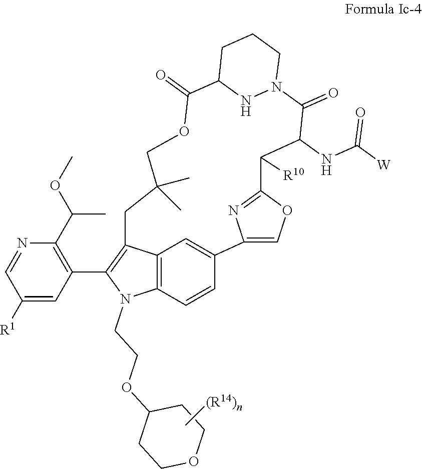

wherein each R¹⁴ is independently C₁-C₃ alkyl; and

In some embodiments, a compound of the disclosure has the structure of Formula Id-4, or a pharmaceutically acceptable salt or a stereoisomer thereof:

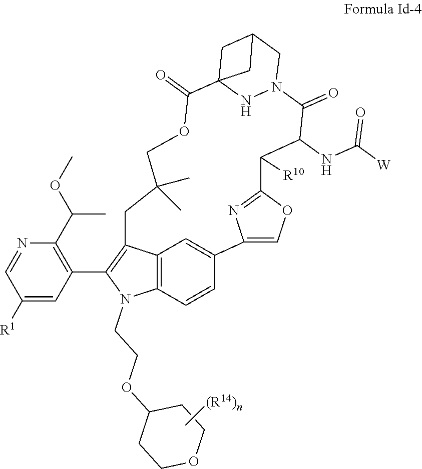

wherein each R¹⁴ is independently C₁-C₃ alkyl; and
n is 0, 1, 2, or 3.

In some embodiments, a compound of the disclosure has the structure of Formula Ie-4, or a pharmaceutically acceptable salt or a stereoisomer thereof:

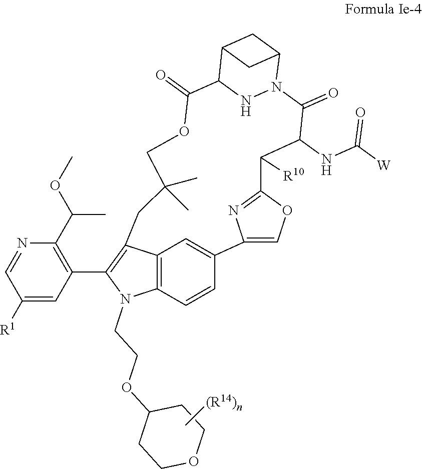

wherein each R¹⁴ is independently C₁-C₃ alkyl; and
n is 0, 1, 2, or 3.

In some embodiments, each R¹⁴ is methyl. In some embodiments, n is 1. In some embodiments, n is 2. In some embodiments, n is 0.

In some embodiments, a compound of the disclosure has the structure of Formula Ic-5:

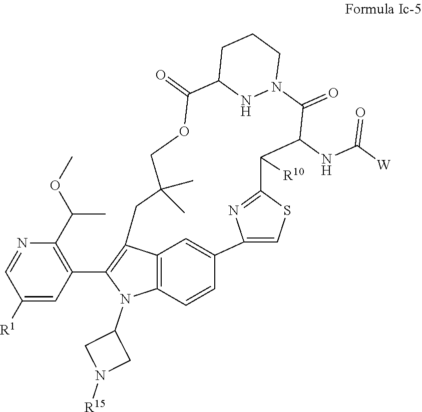

or a pharmaceutically acceptable salt or a stereoisomer thereof, wherein R¹⁵ is C₁-C₃ alkyl or C₃-C₆ cycloalkyl.

In some embodiments, a compound of the disclosure has the structure of Formula Id-5:

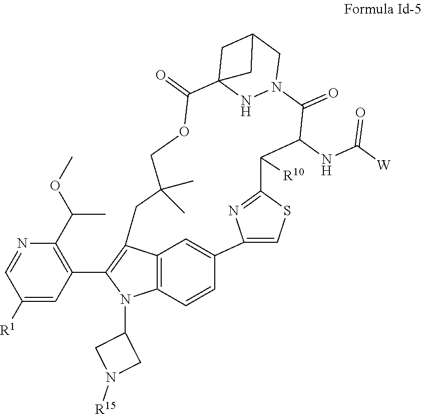

or a pharmaceutically acceptable salt or a stereoisomer thereof, wherein R¹⁵ is C₁-C₃ alkyl or C₃-C₆ cycloalkyl.

In some embodiments, a compound of the disclosure has the structure of Formula Ie-5:

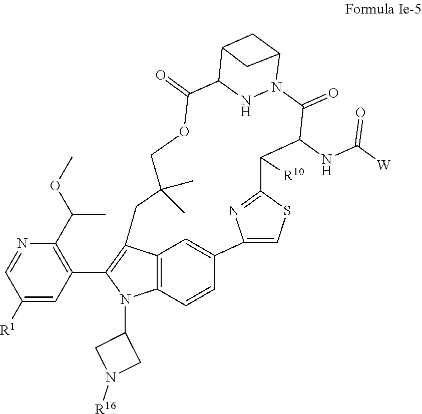

or a pharmaceutically acceptable salt or a stereoisomer thereof, wherein R¹⁵ is C₁-C₃ alkyl or C₃-C₆cycloalkyl.

In some embodiments, a compound of the disclosure has the structure of Formula Ic-6:

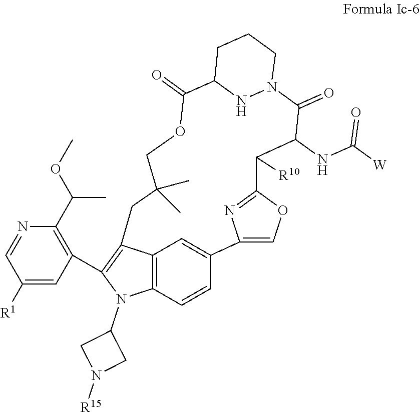

or a pharmaceutically acceptable salt or a stereoisomer thereof, wherein R¹⁵ is C₁-C₃ alkyl or C₃-C₆cycloalkyl.

In some embodiments, a compound of the disclosure has the structure of Formula Id-6:

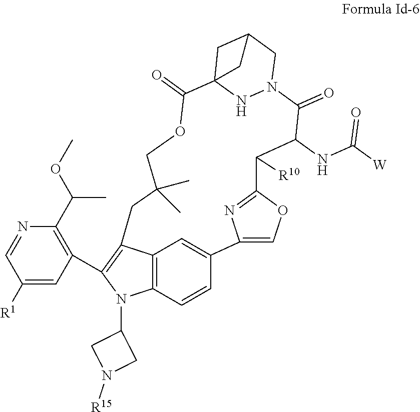

or a pharmaceutically acceptable salt or a stereoisomer thereof, wherein R¹⁵ is C₁-C₃ alkyl or C₃-C₆ cycloalkyl.

In some embodiments, a compound of the disclosure has the structure of Formula Ie-6:

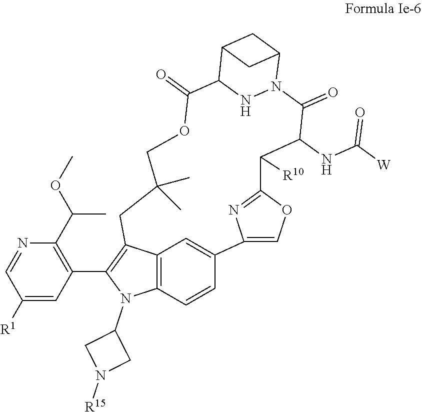

or a pharmaceutically acceptable salt or a stereoisomer thereof, wherein R¹⁵ is C₁-C₃ alkyl or C₃-C₆ cycloalkyl.

In some embodiments, R¹⁵ is C₃-C₆ cycloalkyl. In some embodiments, R¹⁵ is cyclopropyl.

In some embodiments of compounds disclosed herein, R¹ is:

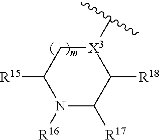

wherein X³ is N or CH;
   m is 1 or 2;
   R¹⁵, R¹⁶, R¹⁷, and R¹⁸ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
   R¹⁶ and R¹⁷ combine with the atoms to which they are attached to form an optionally substituted 3 to 8-membered heterocycloalkyl; or
   R¹⁷ and R¹⁸ combine with the atoms to which they are attached to form an optionally substituted 3 to 8-membered heterocycloalkyl; or
   R¹⁷ and R¹⁵ combine with the atoms to which they are attached to form an optionally substituted 4 to 8-membered heterocycloalkyl.

In some embodiments, R¹ is:

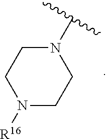

In some embodiments, R¹ is

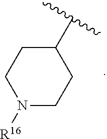

In some embodiments, R¹⁶ is methyl. In some embodiments, R¹⁶ is cyclopropyl.

In some embodiments of compounds of the present disclosure, W is optionally substituted 3 to 10-membered heterocycloalkyl. In some embodiments, W is optionally substituted 4- to 6-membered optionally substituted heterocycloalkyl. In some embodiments, the heterocycloalkyl comprises one oxygen atom. In some embodiments, the heterocycloalkyl comprises one nitrogen atom.

In some embodiments of compounds of the present disclosure, W is optionally substituted 3 to 10-membered heterocycloalkyl. In some embodiments, W is optionally substituted 3- to 6-membered optionally substituted heterocycloalkyl. In some embodiments, W is optionally substituted cyclopropyl.

In some embodiments of compounds of the present disclosure, R¹⁰ is —OR¹¹. In some embodiments, R¹¹ is optionally substituted C₁-C₆ alkyl. In some embodiments, R¹¹ is optionally substituted C₁-C₆ heteroalkyl. In some embodiments, R¹⁰ is —OCH₂CH₃.

In some embodiments of compounds of the present disclosure, R¹⁰ is —NR¹²R¹³. In some embodiments, R¹² and R¹³ combine to form an optionally substituted 3- to 10-membered heterocycloalkyl. In some embodiments, R¹² and R¹³ combine to form an optionally substituted pyrrolidine. In some embodiments, R¹² and R¹³ combine to form an optionally substituted azetidine.

In some embodiments, R¹⁰ is

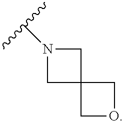 [SMILES: *N1CC2(COC2)C1]

In some embodiments, R¹⁰ is

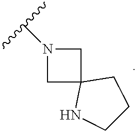 [SMILES: *N1CC2(CCCN2)C1]

In some embodiments, R¹⁰ is

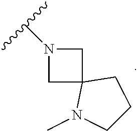 [SMILES: *N1CC2(CCCN2C)C1]

In some embodiments, R¹⁰ is

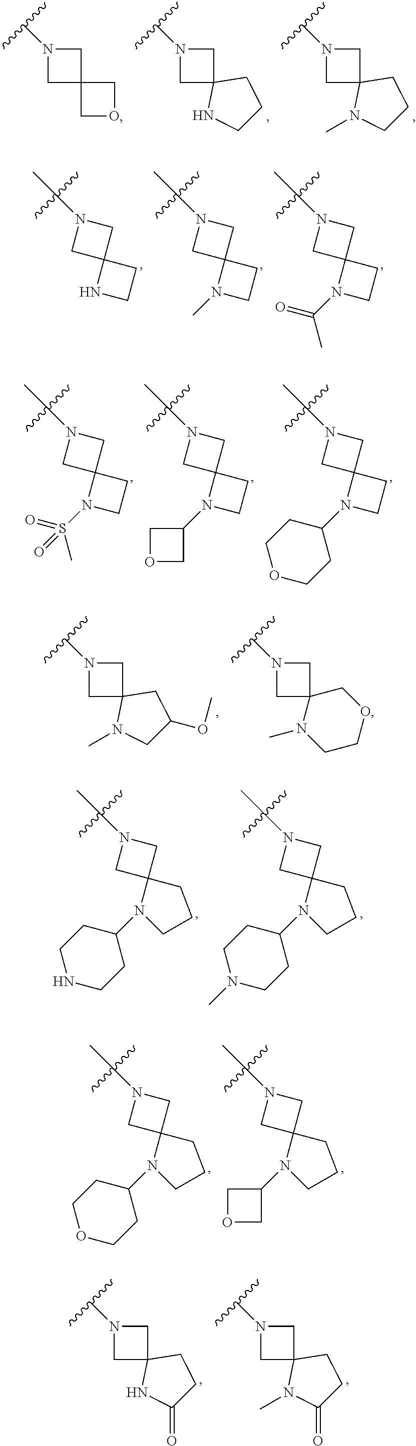

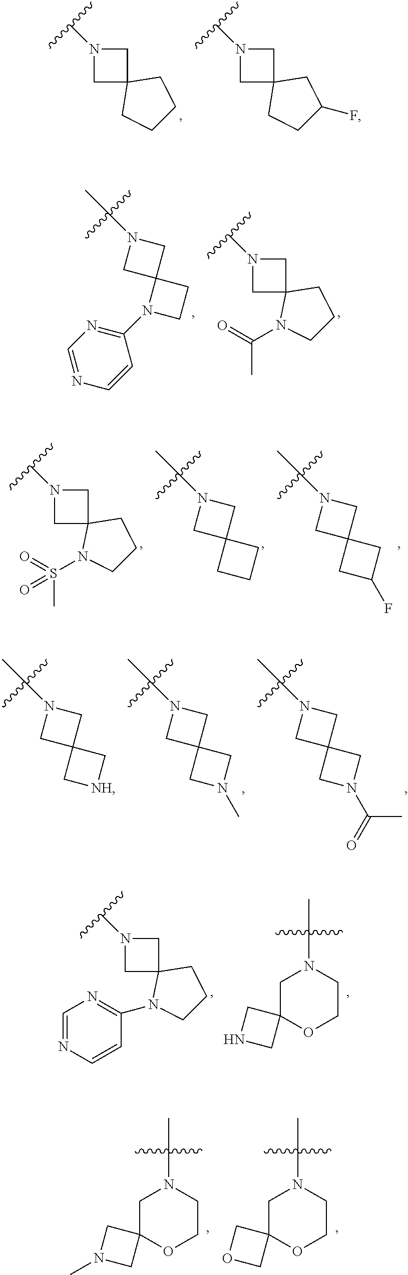

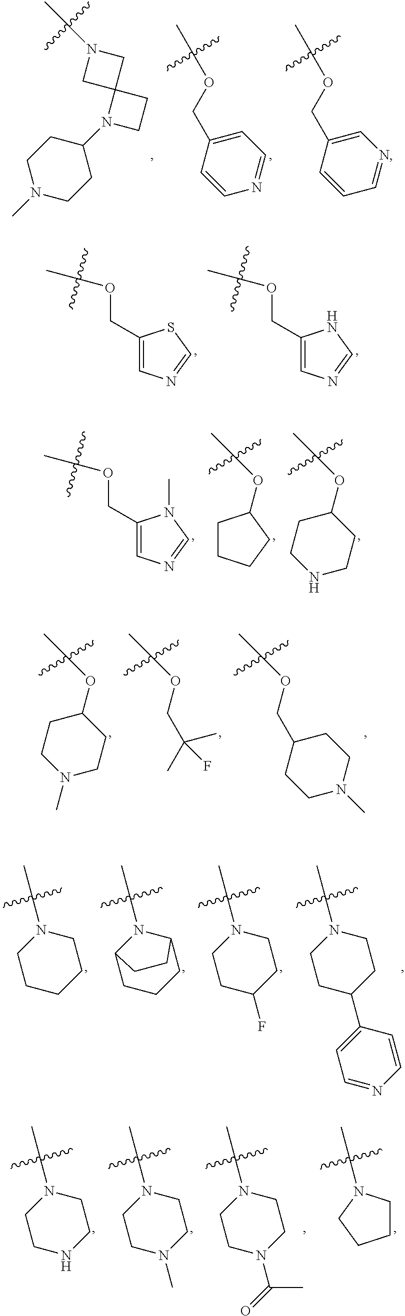

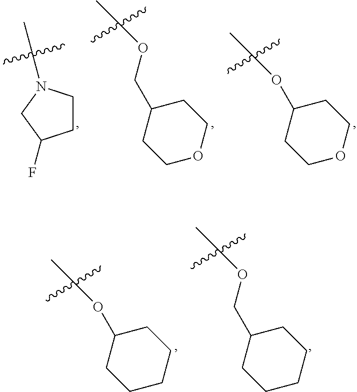 [SMILES: *N1CCC(F)C1 ; *OCC1CCOCC1 ; *OC1CCOCC1 ; *OC1CCCCC1 ; *OCC1CCCCC1]

—OCH₃, —N(CH₃)₂, —OCH═CH(CH₃)₂, or —OCH₂SO₂CH₃, each of which is optionally substituted.

In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present Invention

| Ex# | Structure |
|---|---|
| A1 | 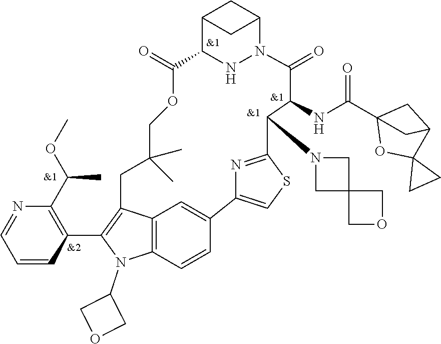 |
| A2 | 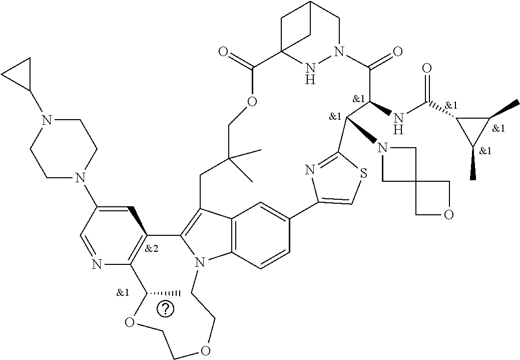 |
| A3 | 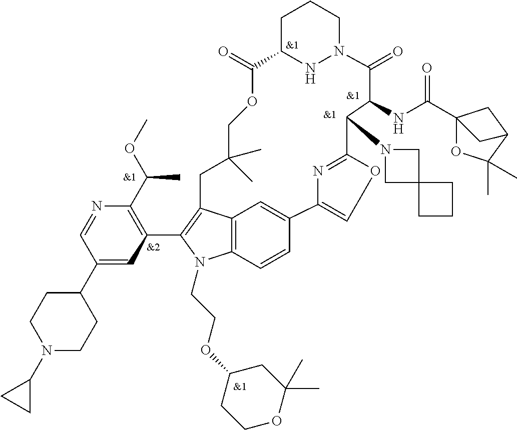 |
| A4 | 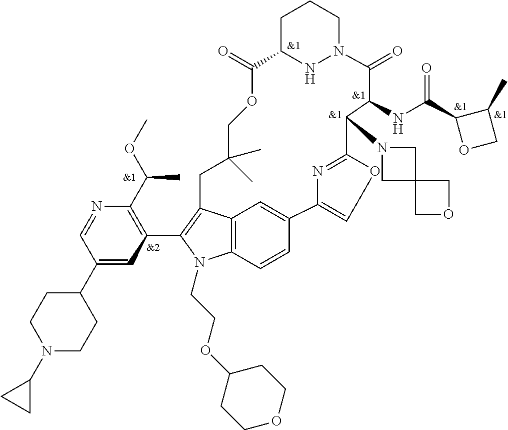 |
| A5 | 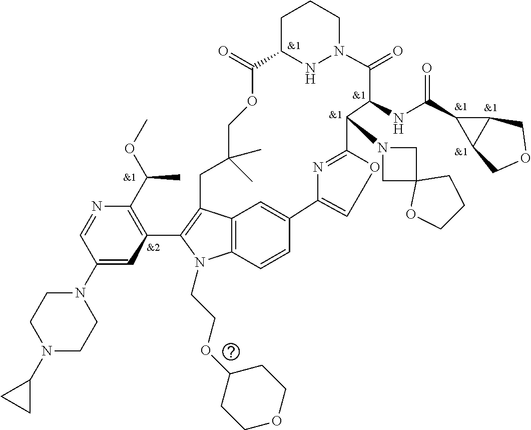 |
| A6 | 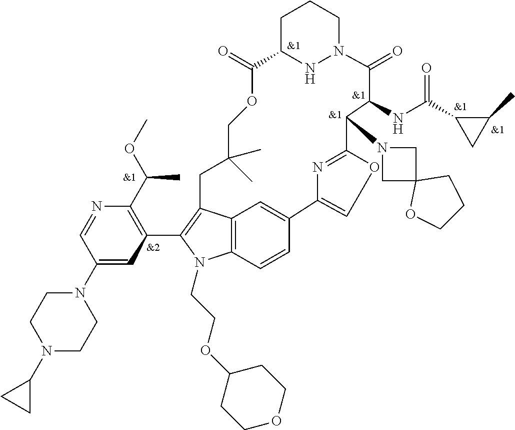 |
| A7 | 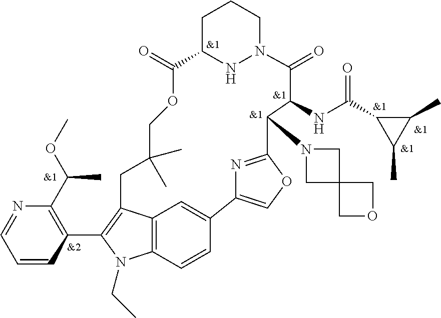 |
| A8 | 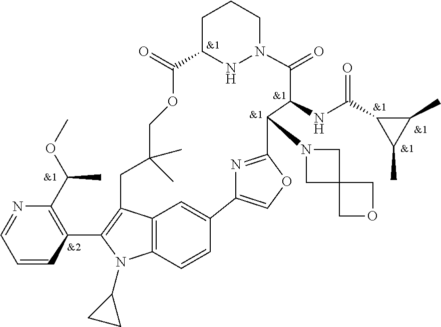 |
| A9 | 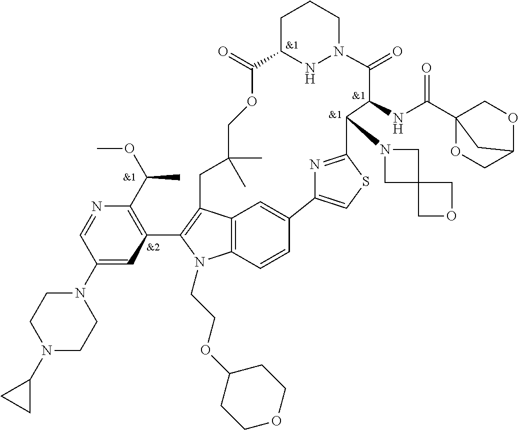 |
| A10 | 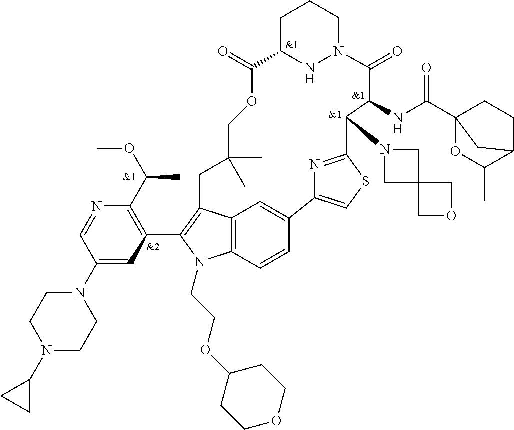 |
| A11 | 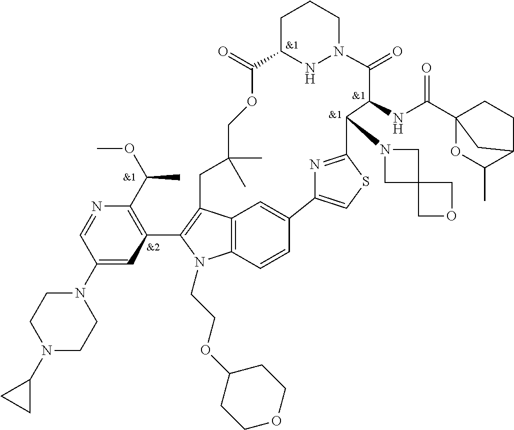 |
| A12 | 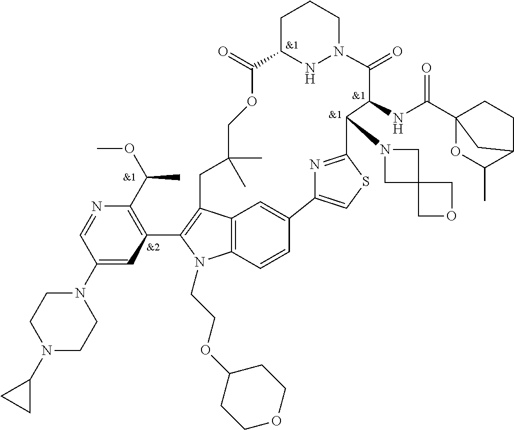 |
| A13 | 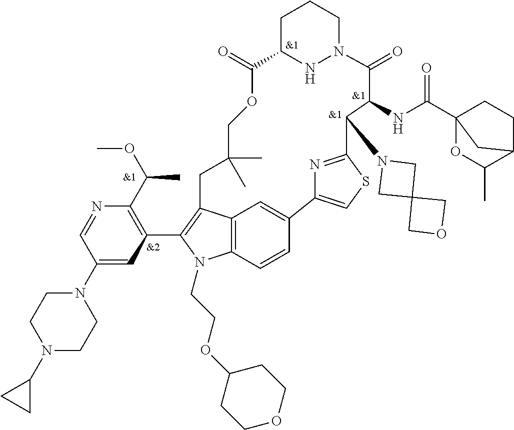 |
| A14 | 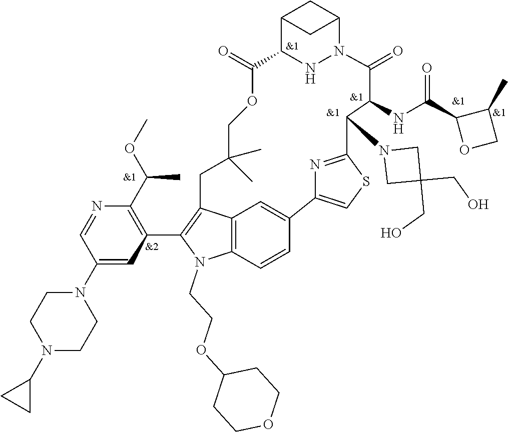 |
| A15 | 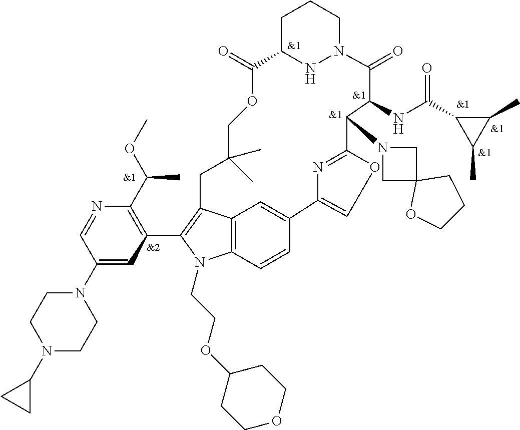 |
| A16 | 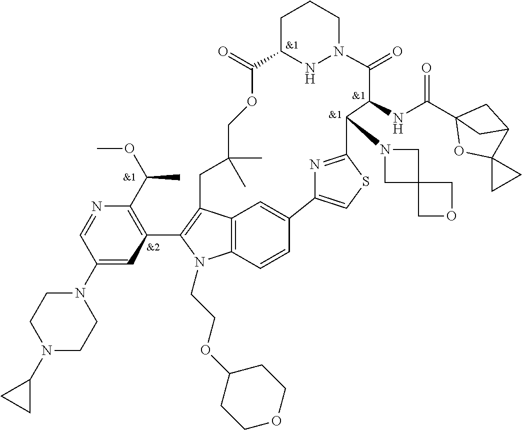 |
| A17 | 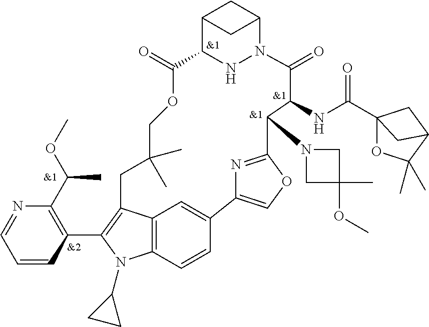 |
| A18 | 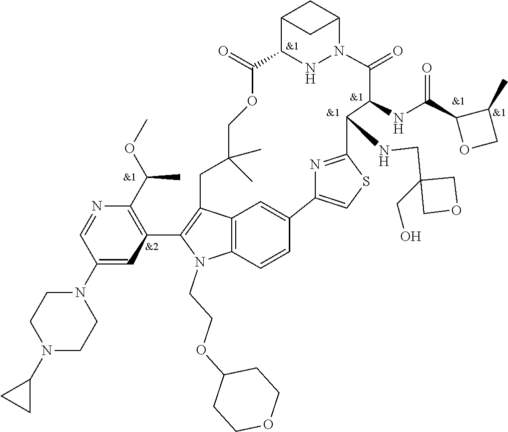 |
| A19 | 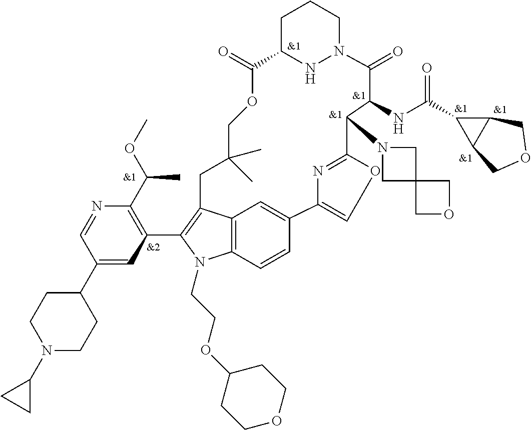 |
| A20 | 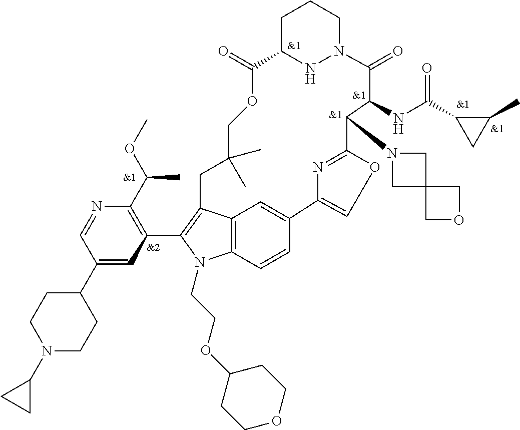 |
| A21 | 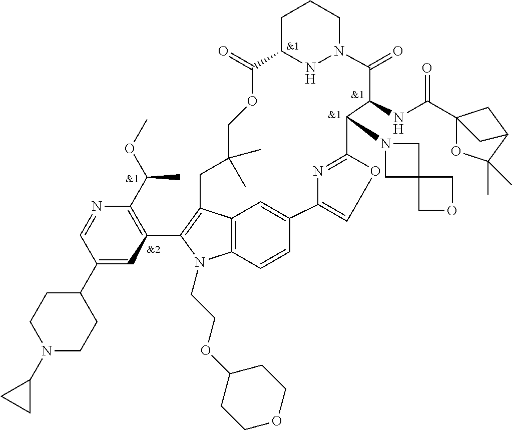 |
| A22 | 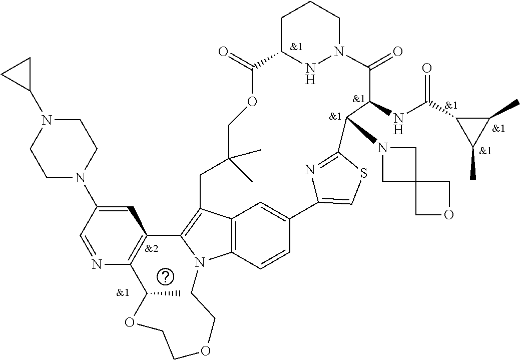 |
| A23 | 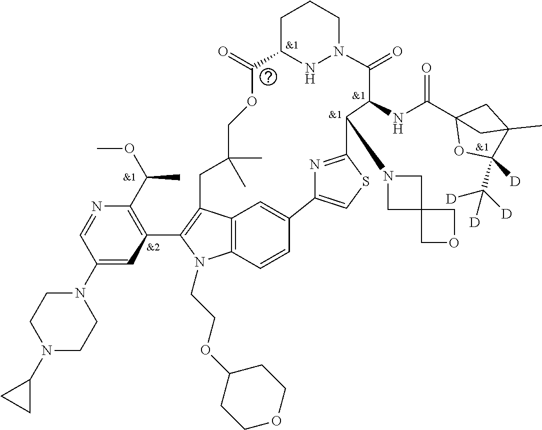 |
| A24 | 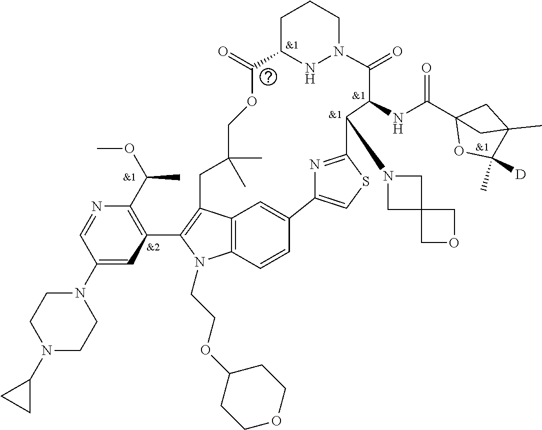 |
| A25 | 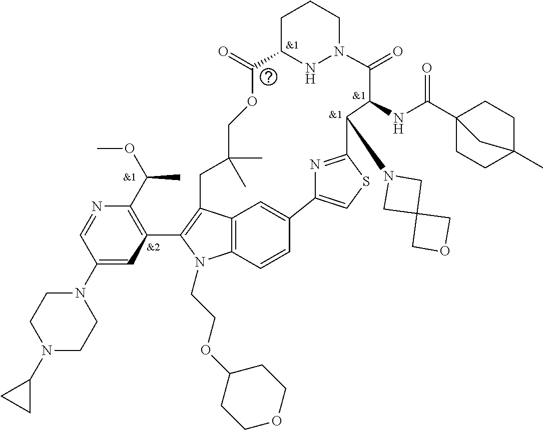 |
| A26 | 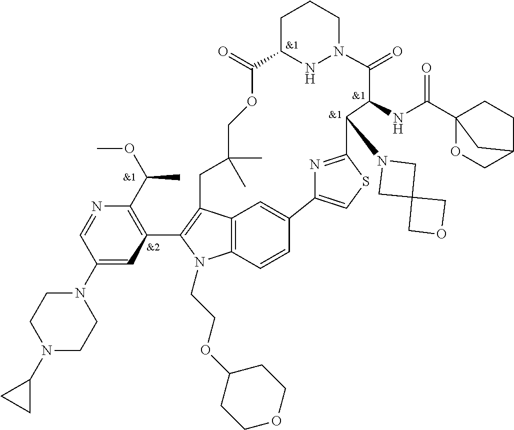 |
| A27 | 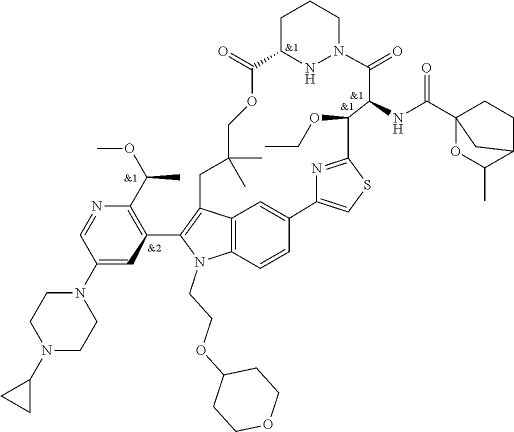 |
| A28 | 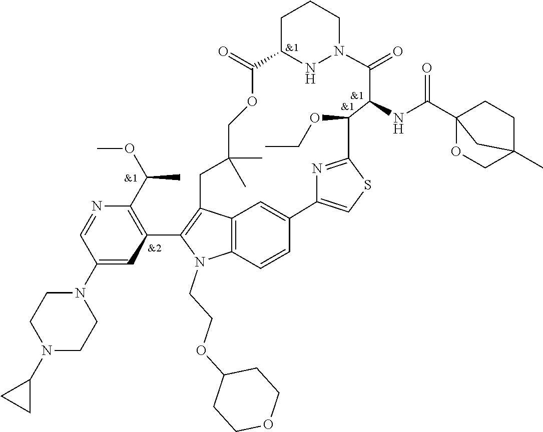 |
| A29 | 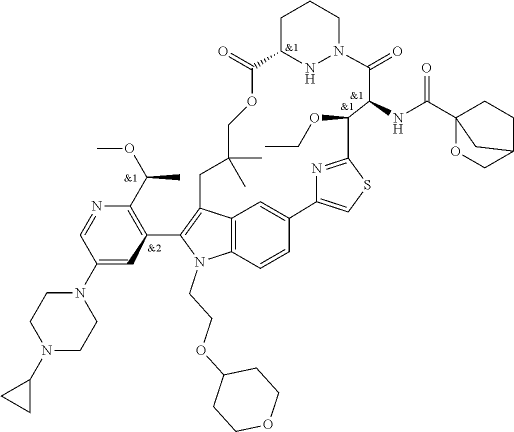 |
| A30 | 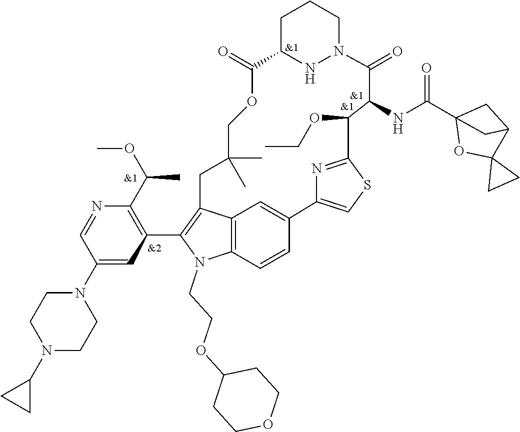 |
| A31 | 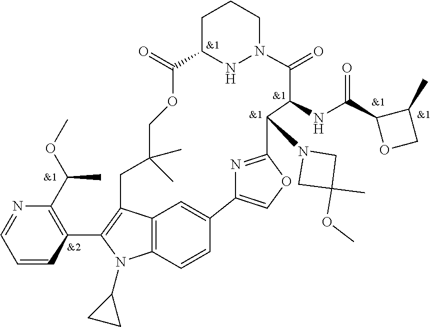 |
| A32 | 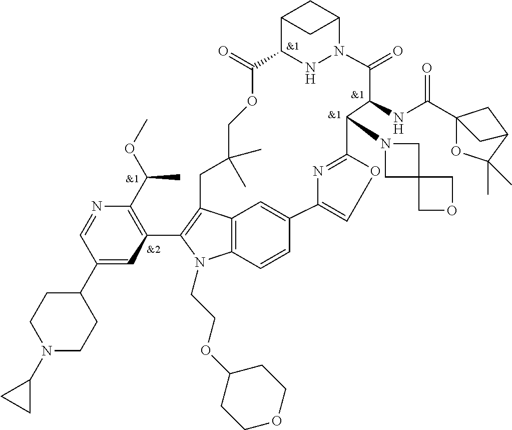 |
| A33 | 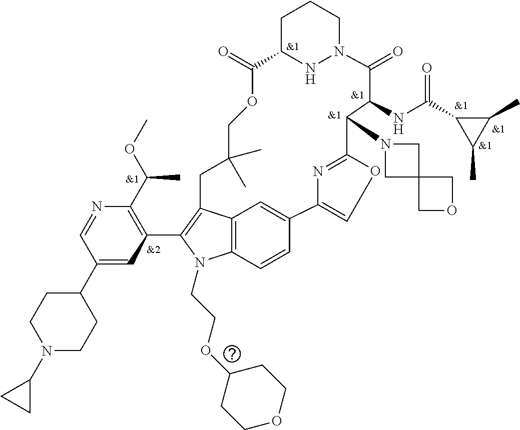 |
| A34 | 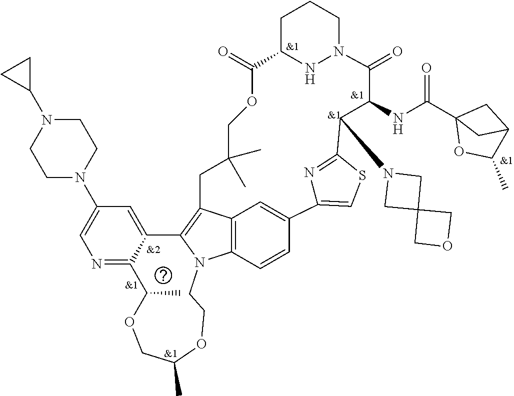 |
| A35 | 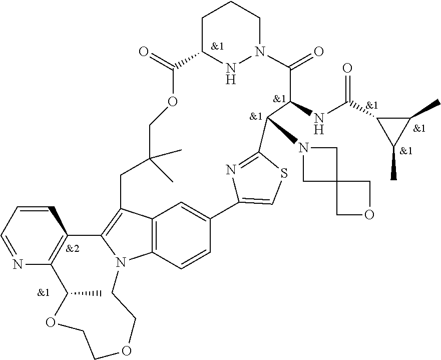 |
| A36 | 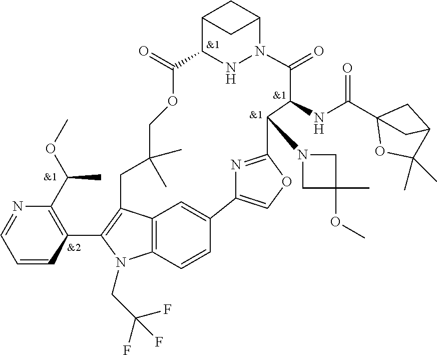 |
| A37 | 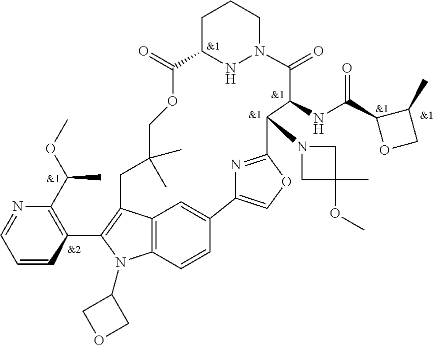 |
| A38 | 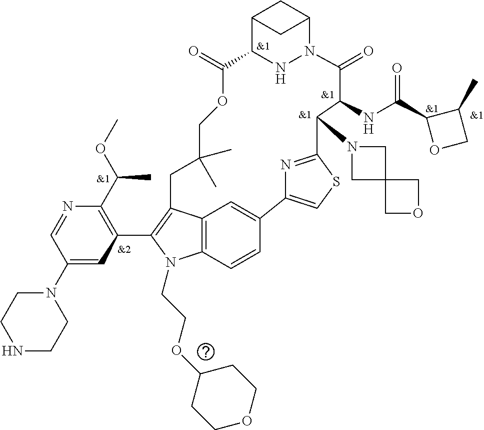 |
| A39 | 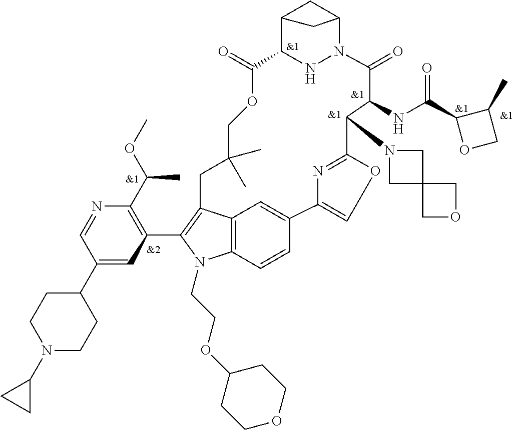 |
| A40 | 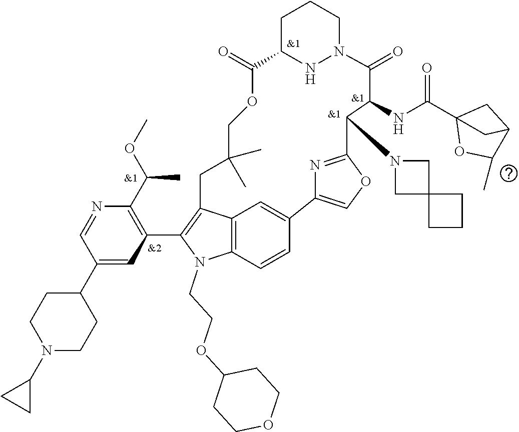 |
| A41 | 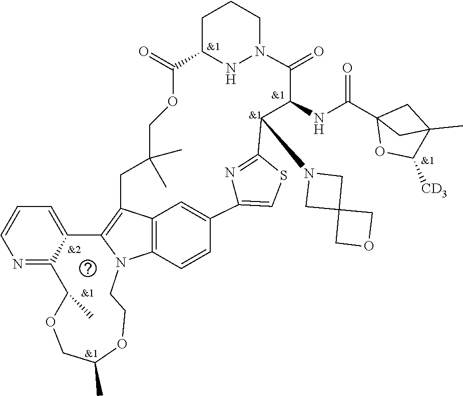 |
| A42 | 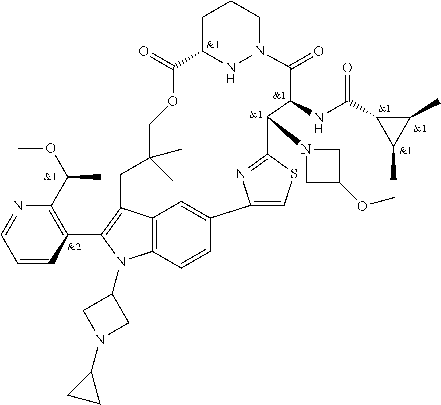 |
| A43 | 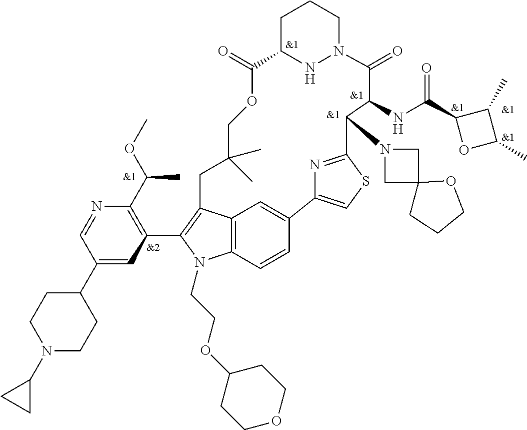 |
| A44 | 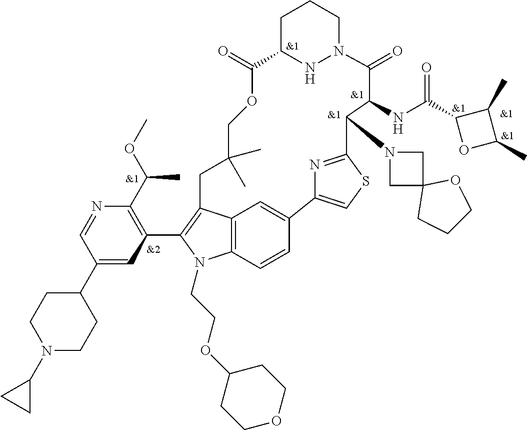 |
| A45 | 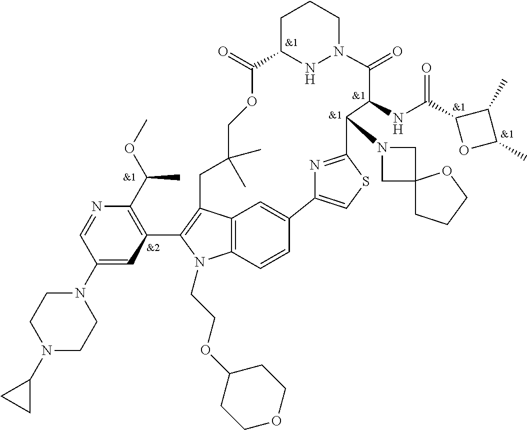 |
| A46 | 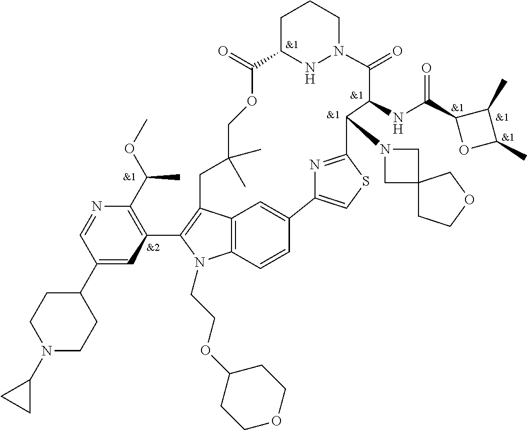 |
| A47 | 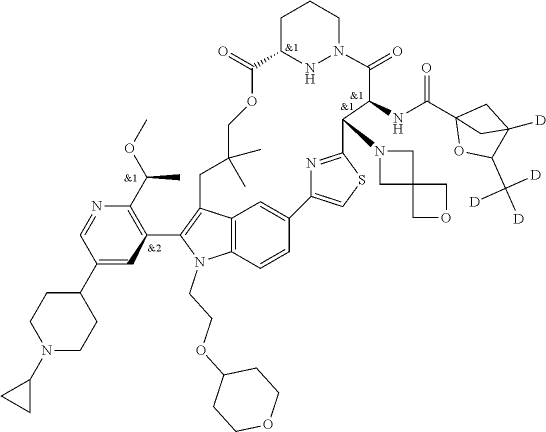 |
| A48 | 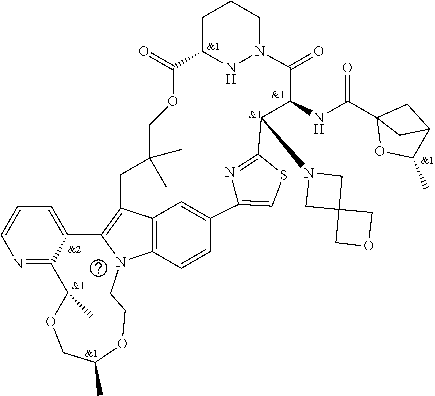 |
| A49 | 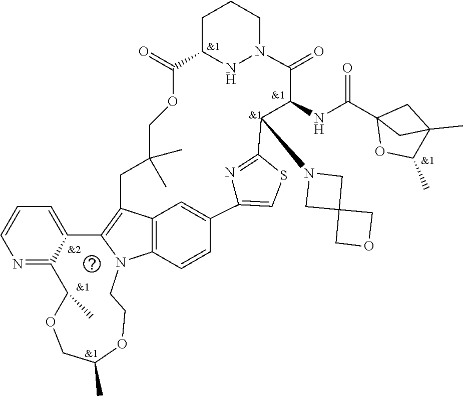 |
| A50 | 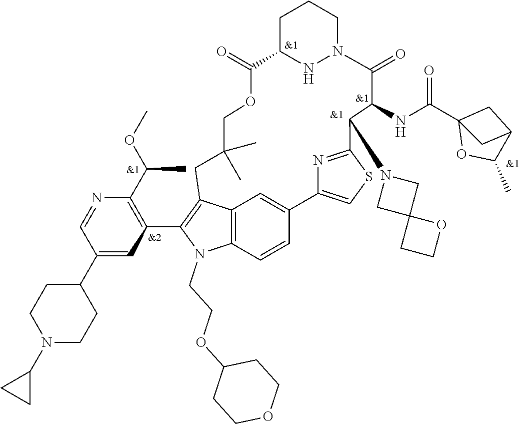 |
| A51 | 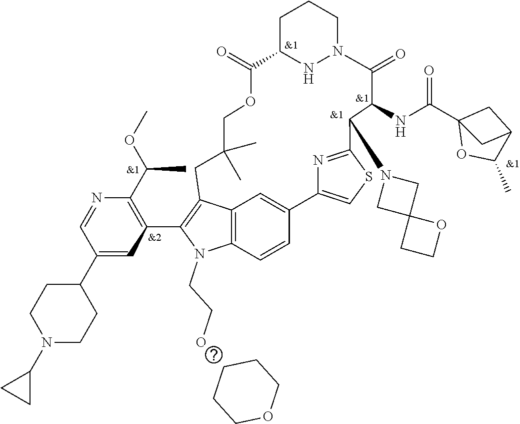 |
| A52 | 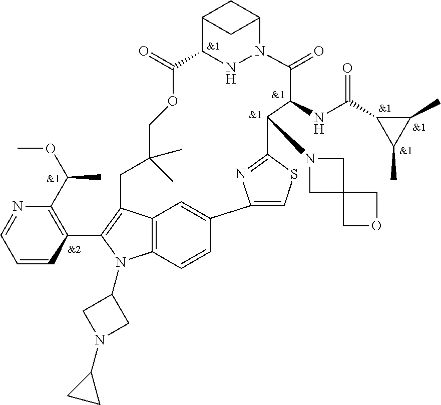 |
| A53 | 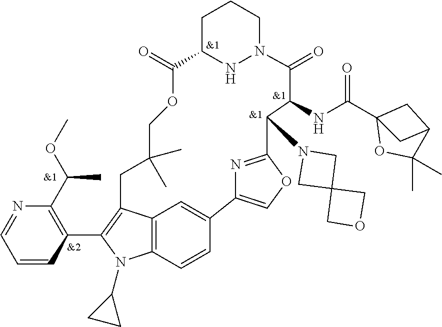 |
| A54 | 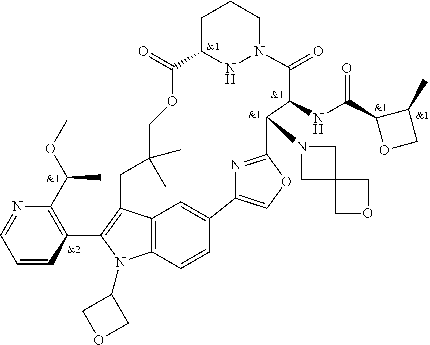 |
| A55 | 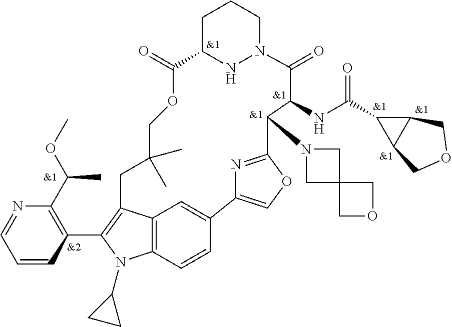 |
| A56 | 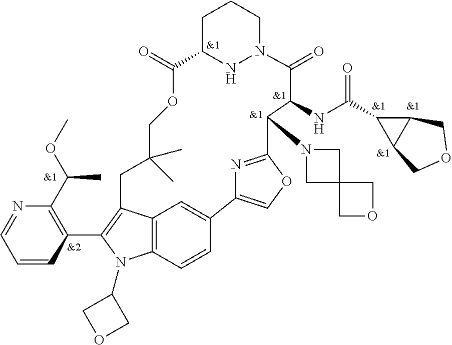 |
| A57 | 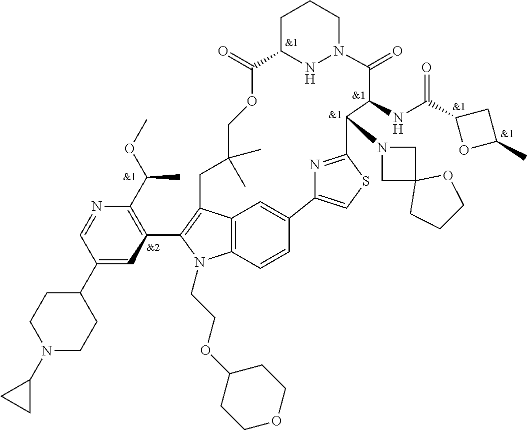 |
| A58 | 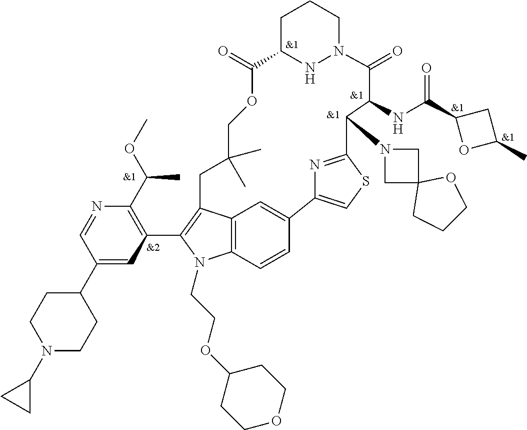 |
| A59 | 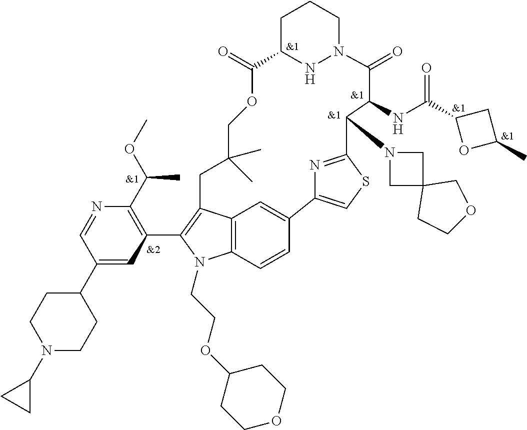 |
| A60 | 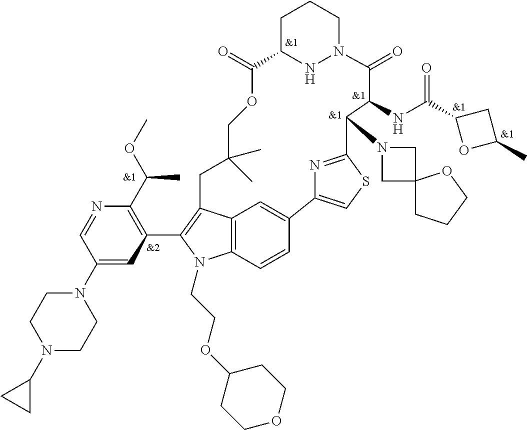 |
| A61 | 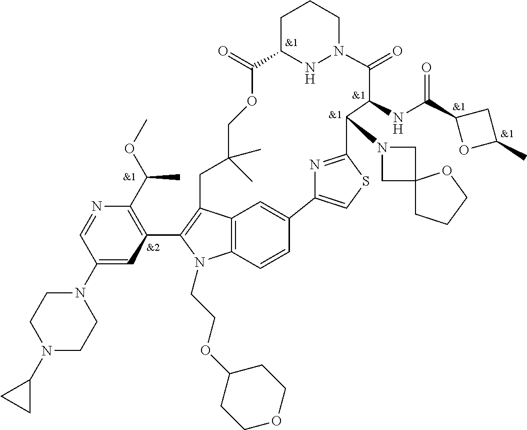 |
| A62 | 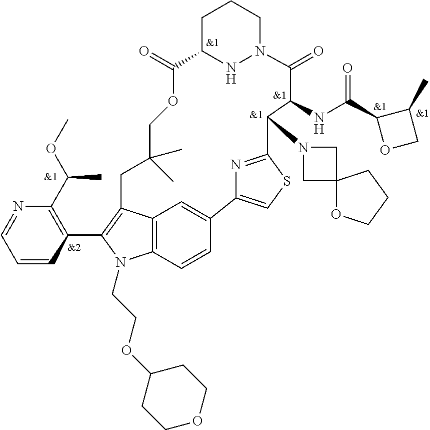 |
| A63 | 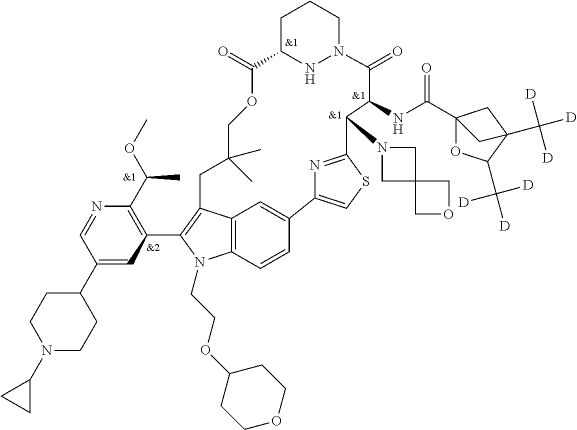 |
| A64 | 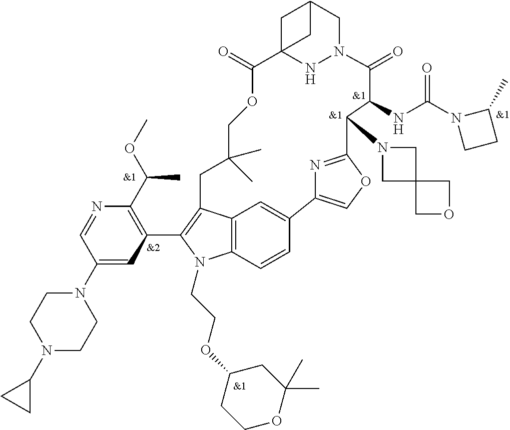 |
| A65 | 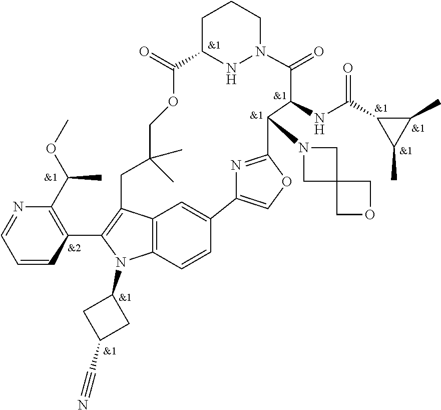 |
| A66 | 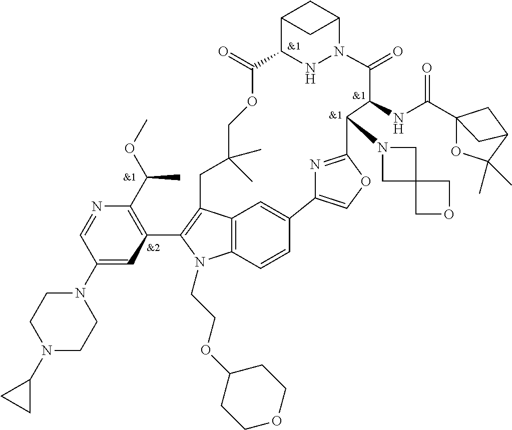 |
| A67 | 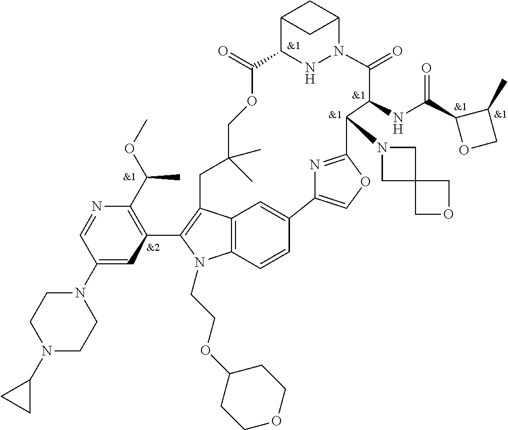 |
| A68 | 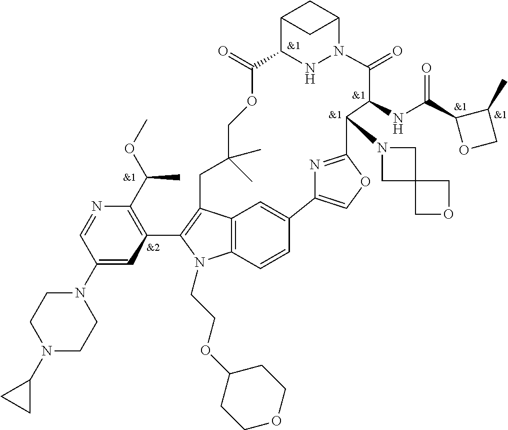 |
| A69 | 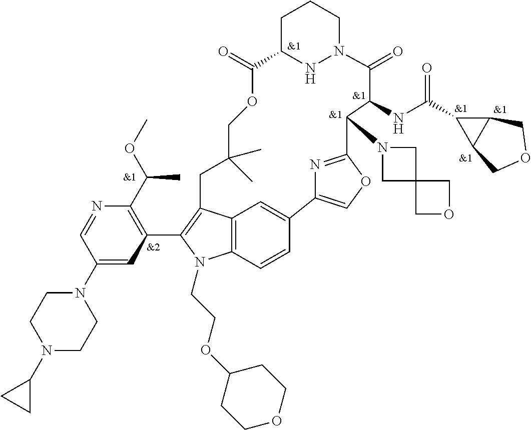 |
| A70 | 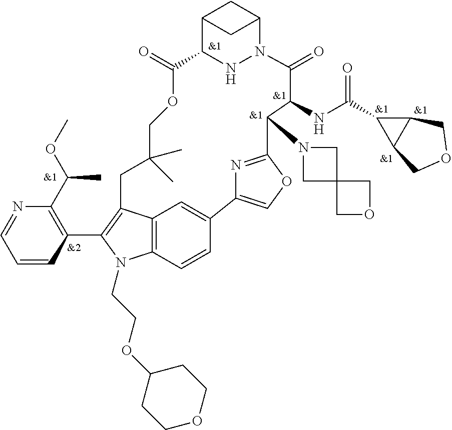 |
| A71 | 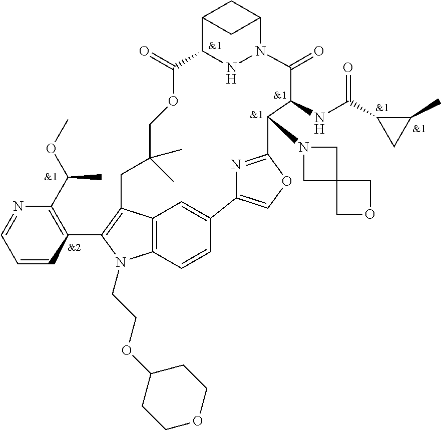 |
| A72 | 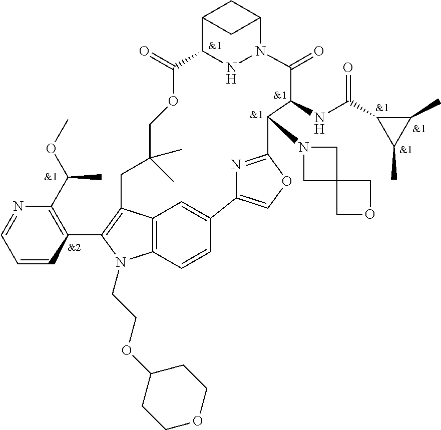 |
| A73 | 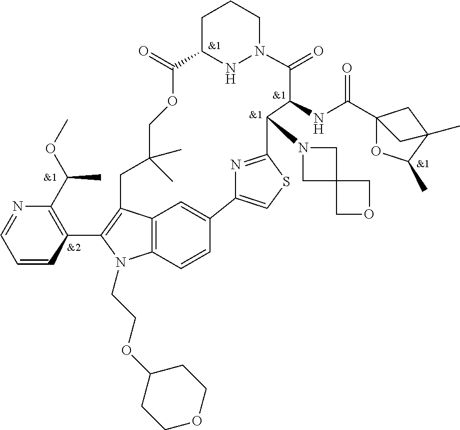 |
| A74 | 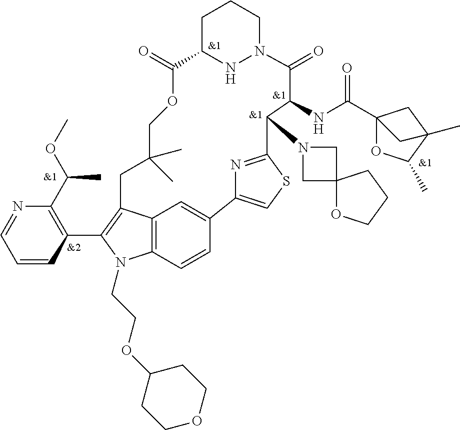 |
| A75 | 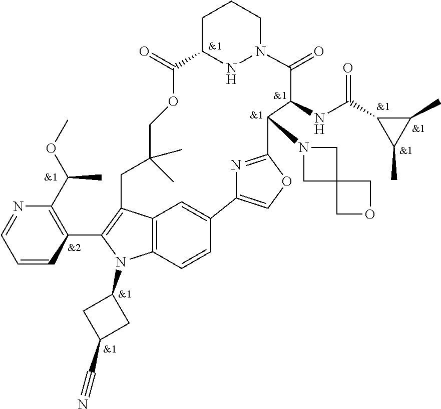 |
| A76 | 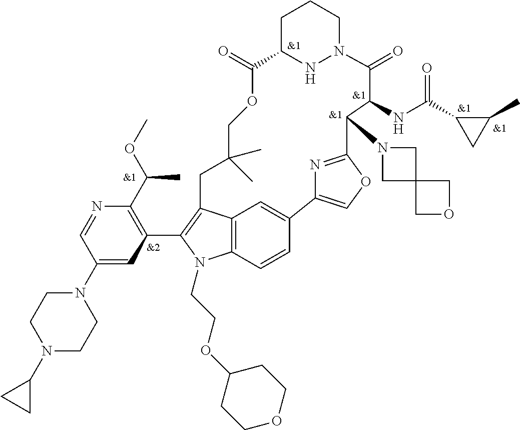 |
| A77 | 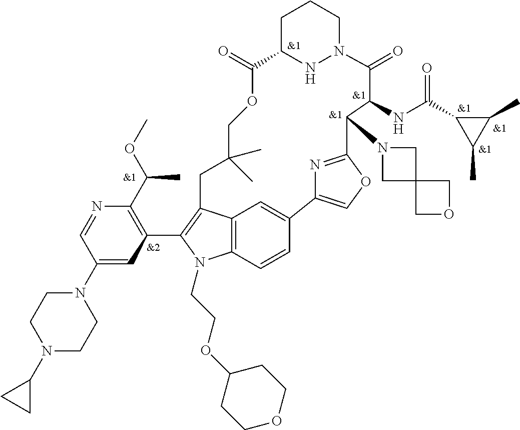 |
| A78 | 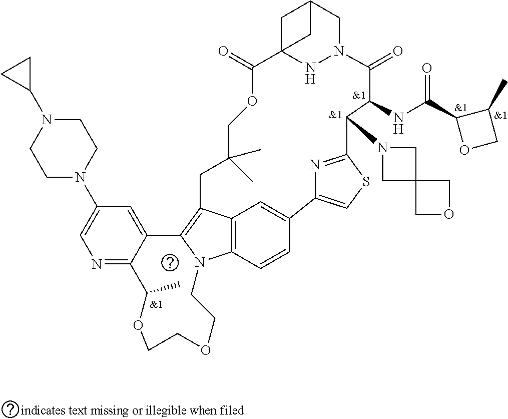 [SMILES: C[C@@H]1OCCOCCn2c(c3c4cc(ccc42)-c2csc(n2)[C@@H](N2CC4(COC4)C2)[C@H](NC(=O)[C@@H]2OC[C@@H]2C)C(=O)N2CC4CC(C4)(N2)C(=O)OCC(C)(C)C3)-c2cc(N3CCN(C4CC4)CC3)cnc21] |
| A79 | 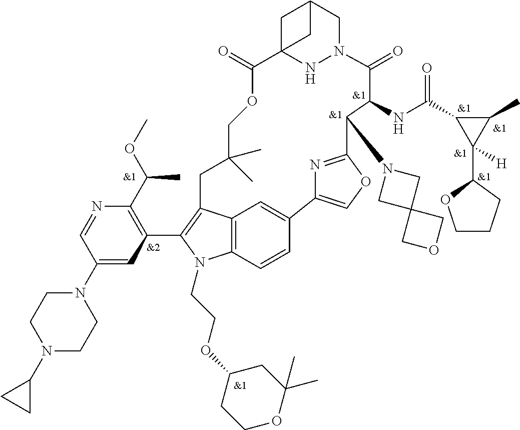 |
| A80 | 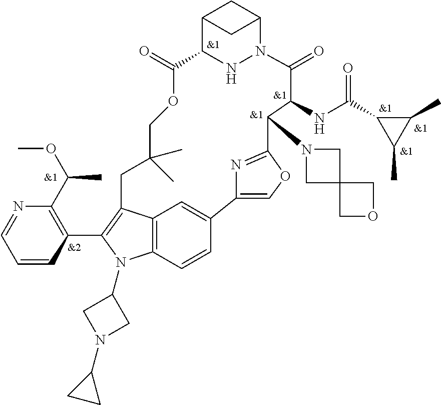 |
| A81 | 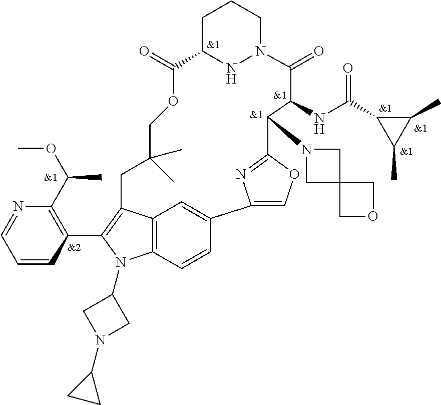 |
| A82 | 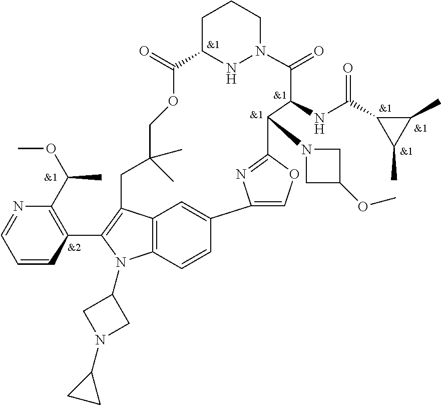 |
| A83 | 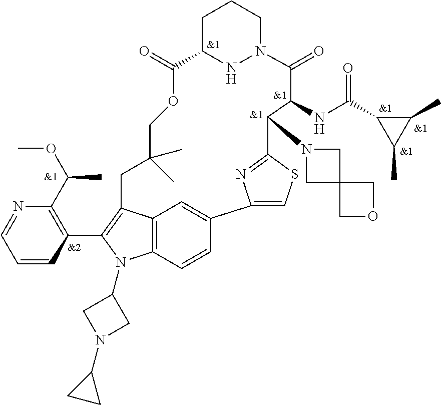 |
| A84 | 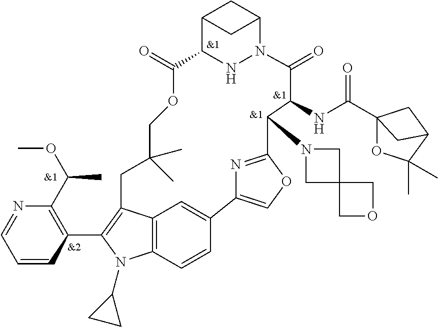 |
| A85 | 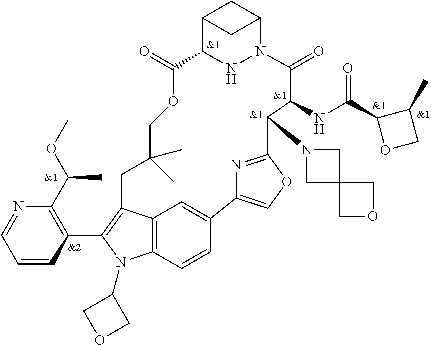 |
| A86 | 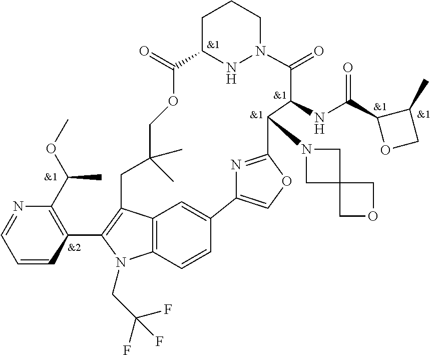 |
| A87 | 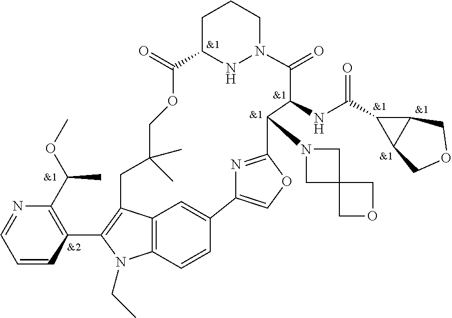 |
| A88 | 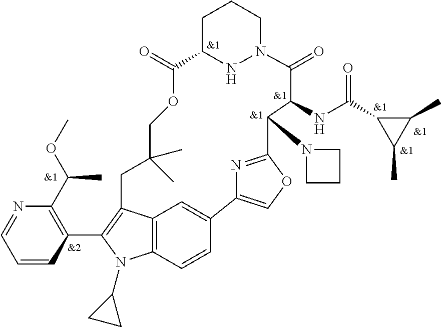 |
| A89 | 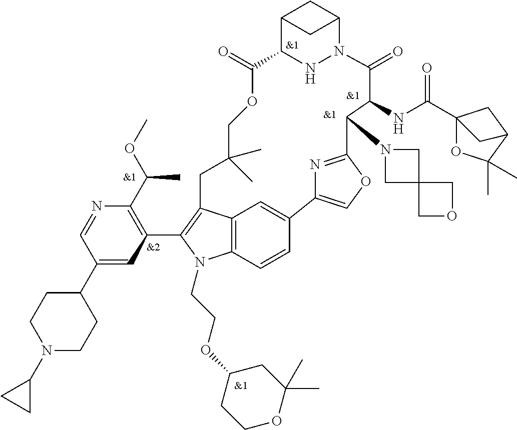 |
| A90 | 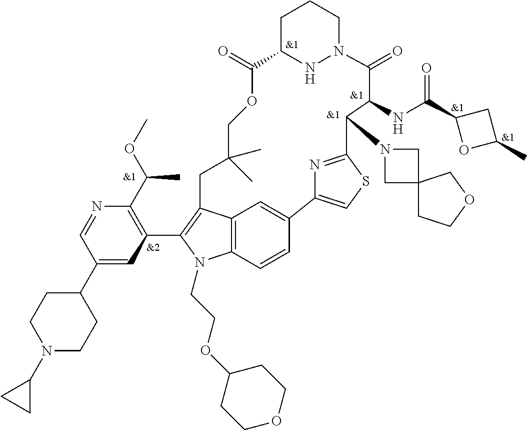 |
| A91 | 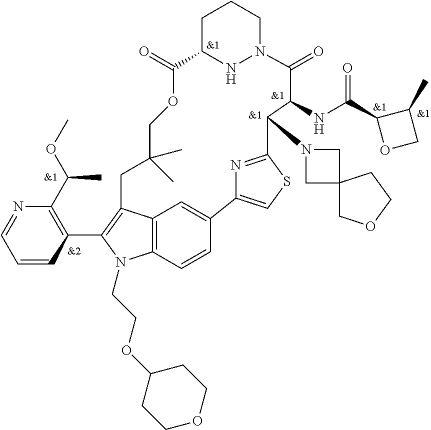 |
| A92 | 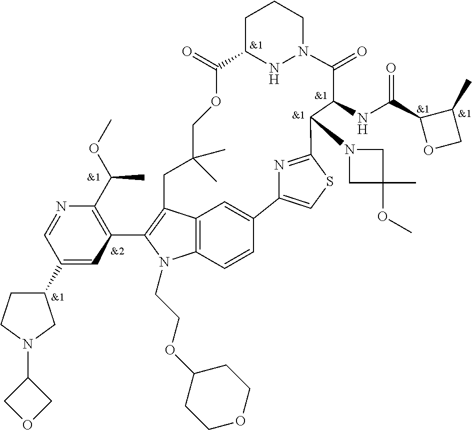 |
| A93 | 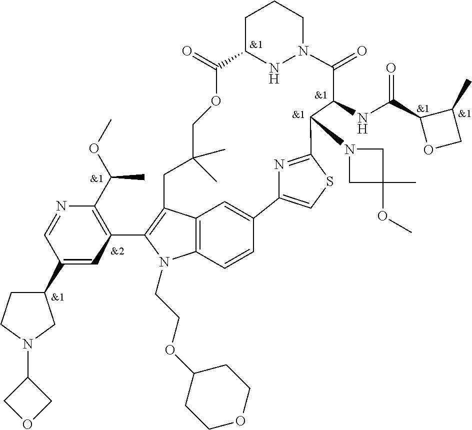 |
| A94 | 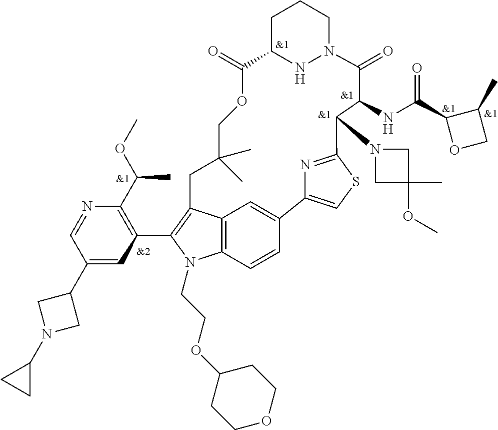 |
| A95 | 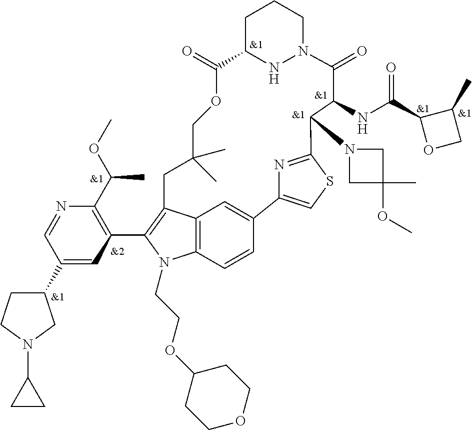 |
| A96 | 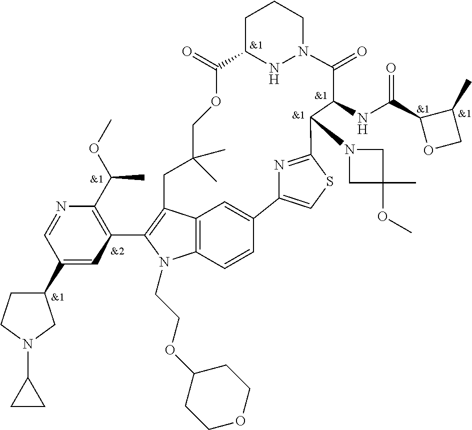 |
| A97 | 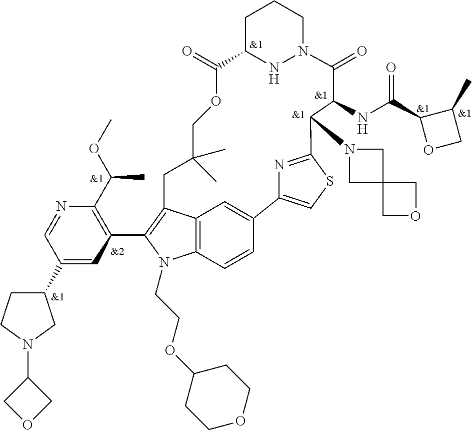 |
| A98 | 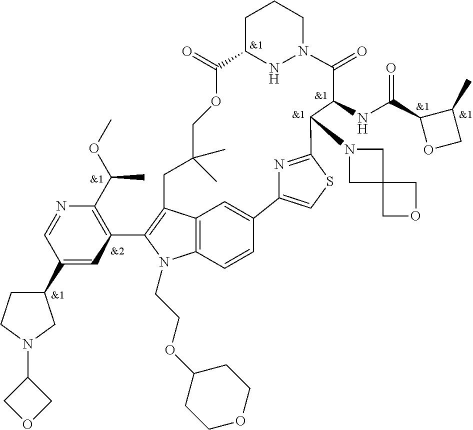 |
| A99 | 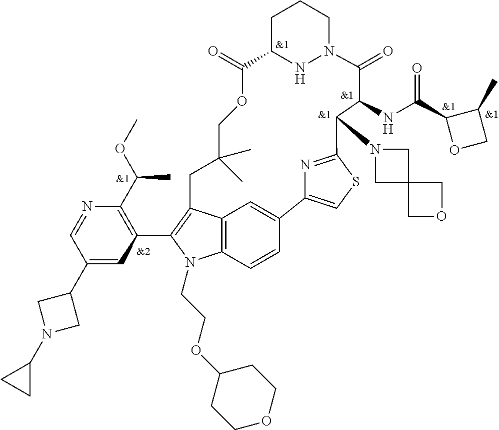 |
| A100 | 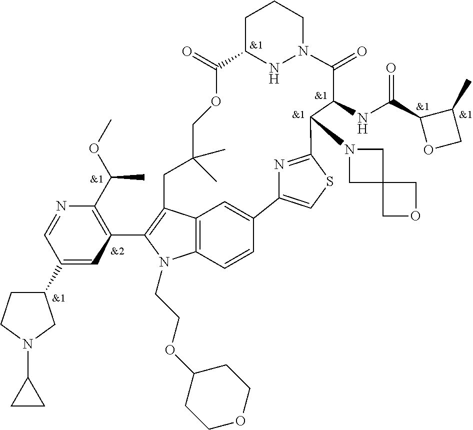 |
| A101 | 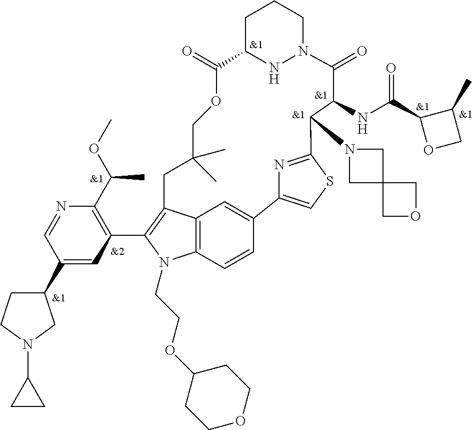 |
| A102 | 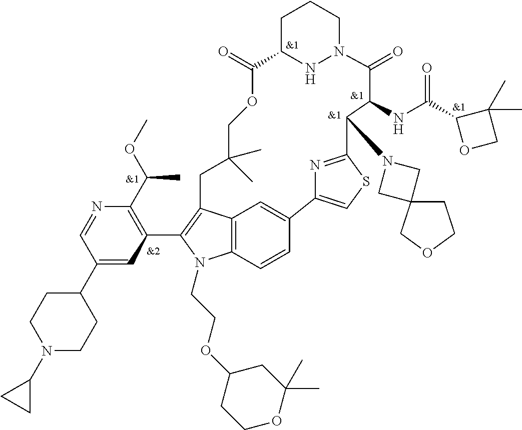 |
| A103 | 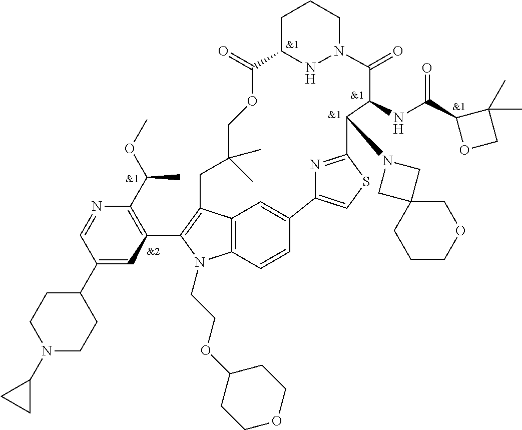 |
| A104 | 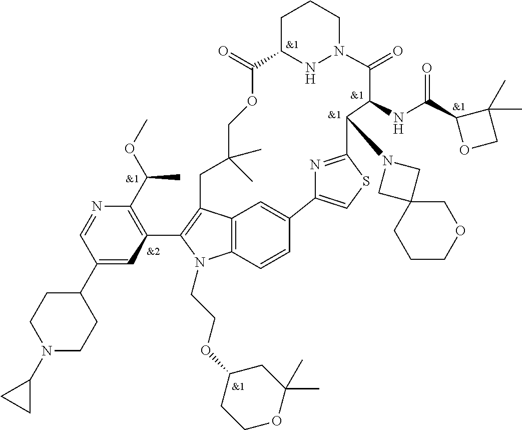 |
| A105 | 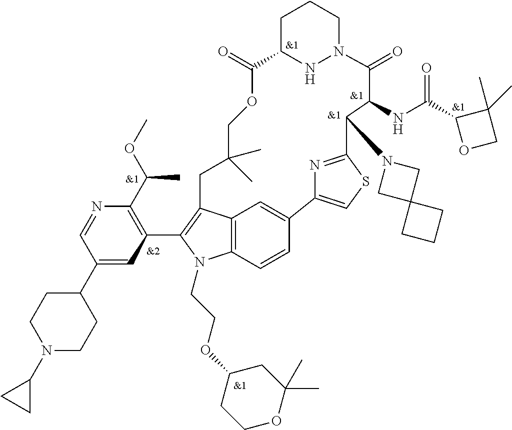 |
| A106 | 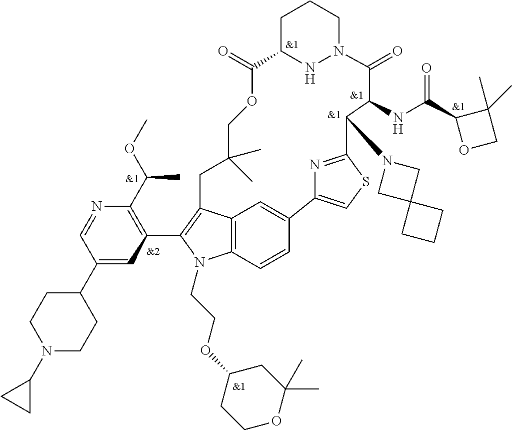 |
| A107 | 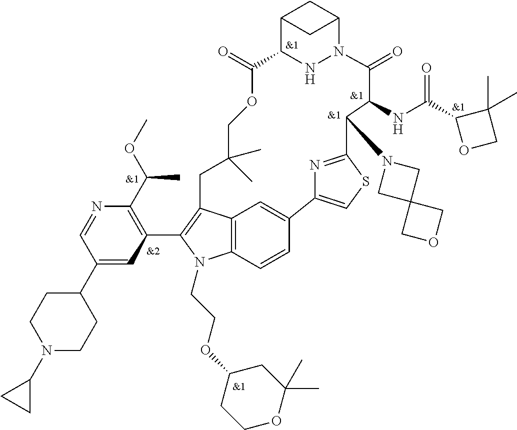 |
| A108 | 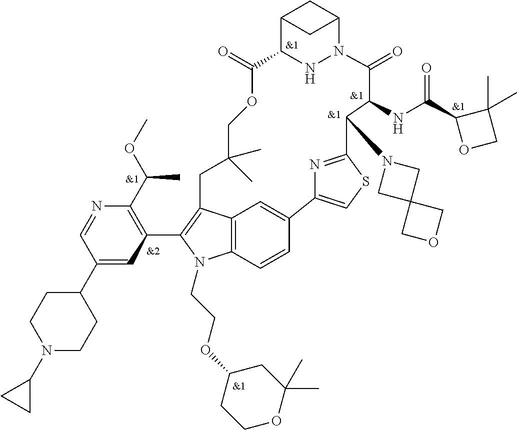 |
| A109 | 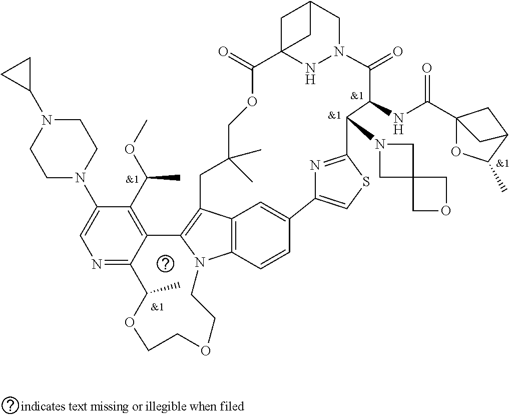 [SMILES: CO[C@@H](C)c1c(N2CCN(C3CC3)CC2)cnc2c1-c1c3c4cc(ccc4n1CCOCCO[C@H]2C)-c1csc(n1)[C@@H](N1CC2(COC2)C1)[C@H](NC(=O)C12CC(C1)[C@H](C)O2)C(=O)N1CC2CC(C2)(N1)C(=O)OCC(C)(C)C3] |
| A110 | 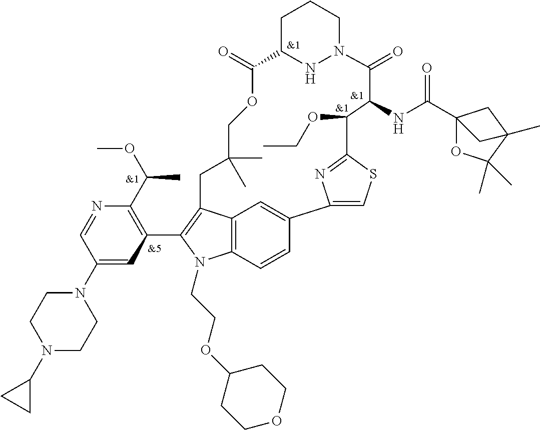 |
| A111 | 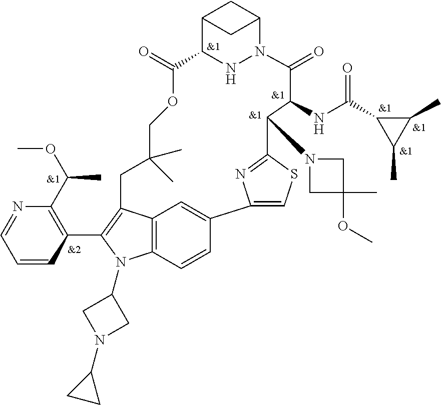 |
| A112 | 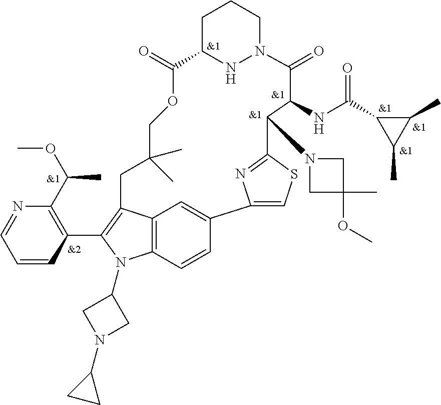 |
| A113 | 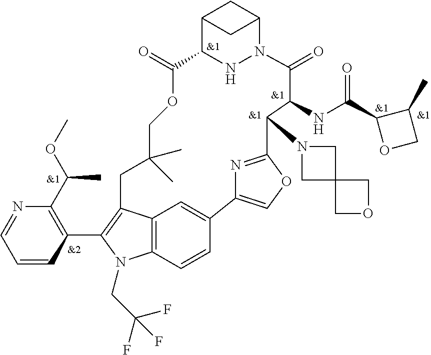 |
| A114 | 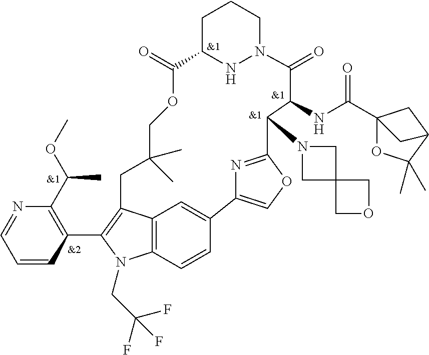 |
| A115 | 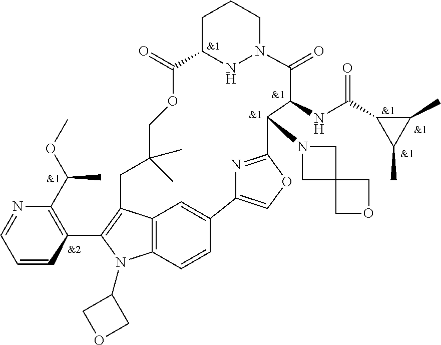 |
| A116 | 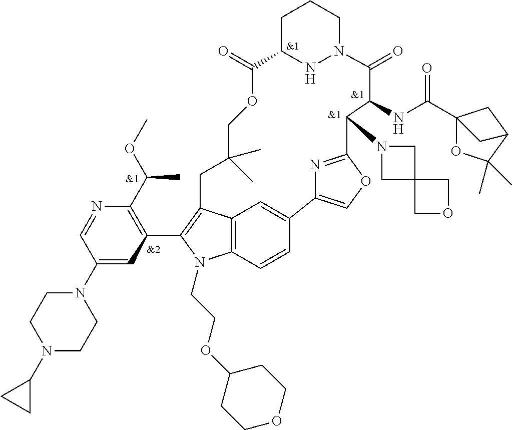 |
| A117 | 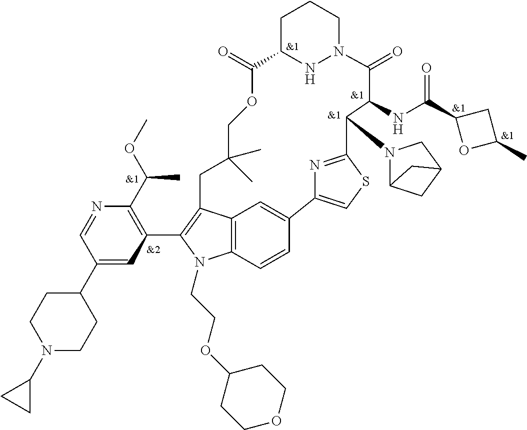 |
| A118 | 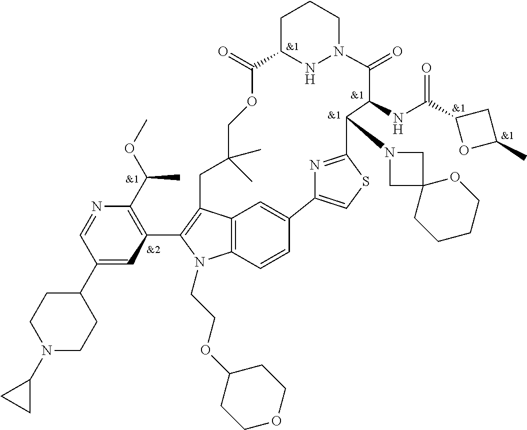 |
| A119 | 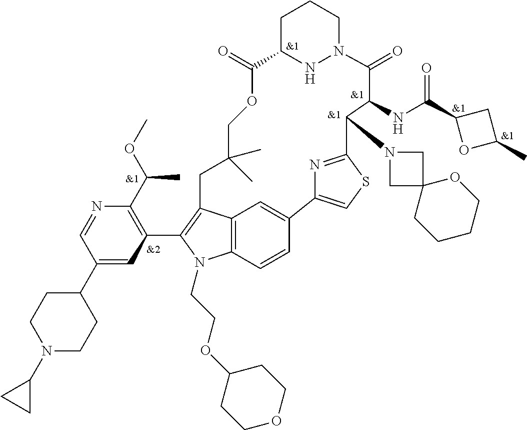 |
| A120 | 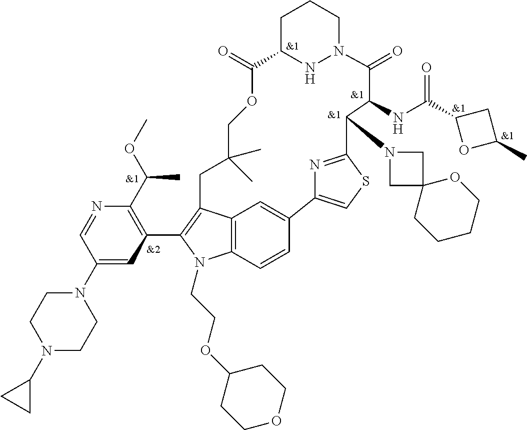 |
| A121 | 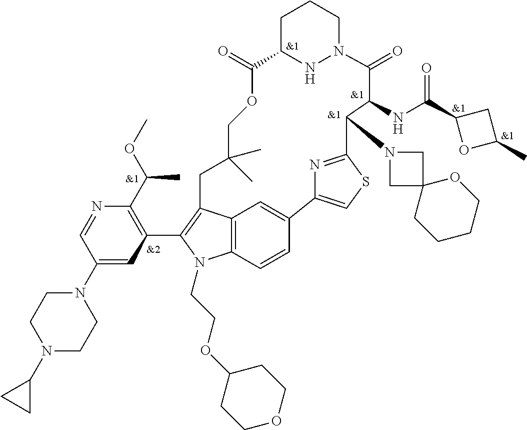 |
| A122 | 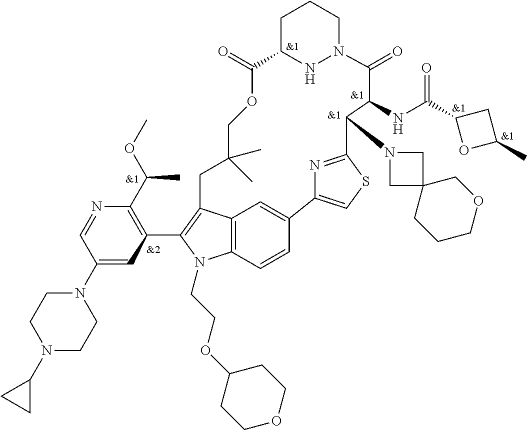 |
| A123 | 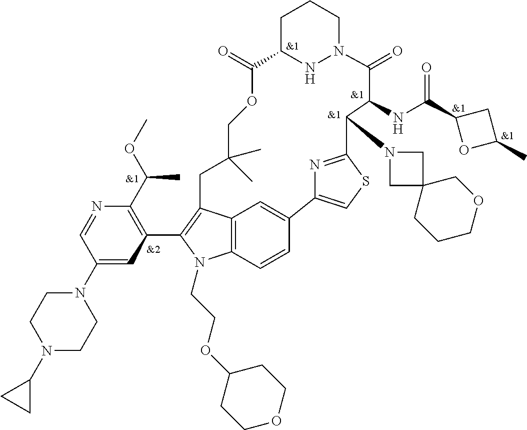 |
| A124 | 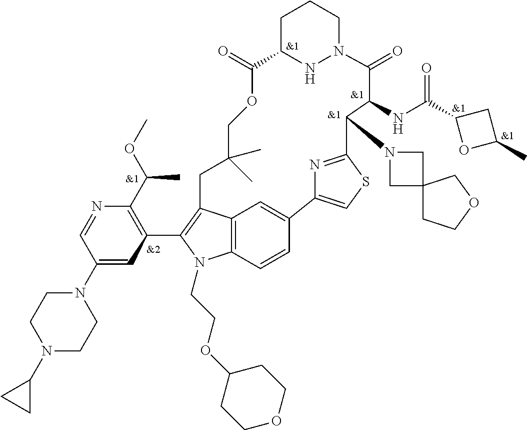 |
| A125 | 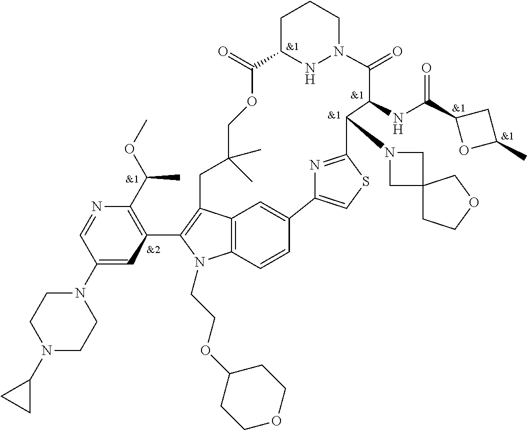 |
| A126 | 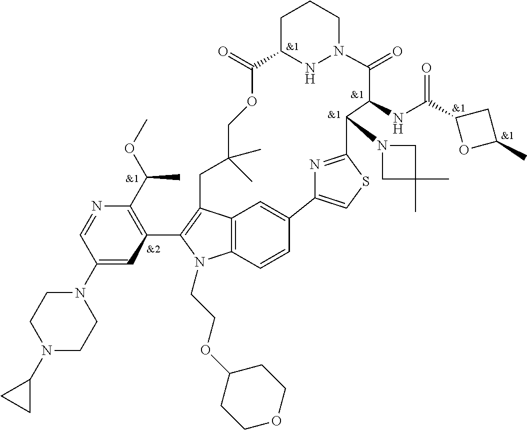 |
| A127 | 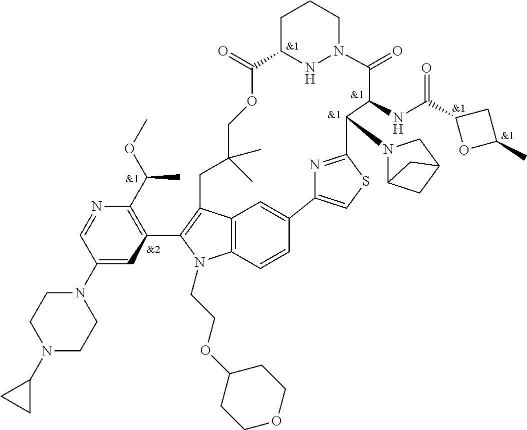 |
| A128 | 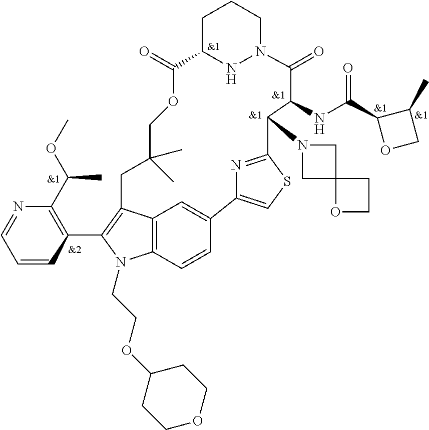 |
| A129 | 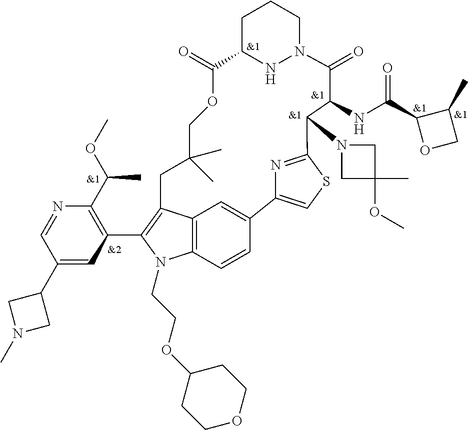 |
| A130 | 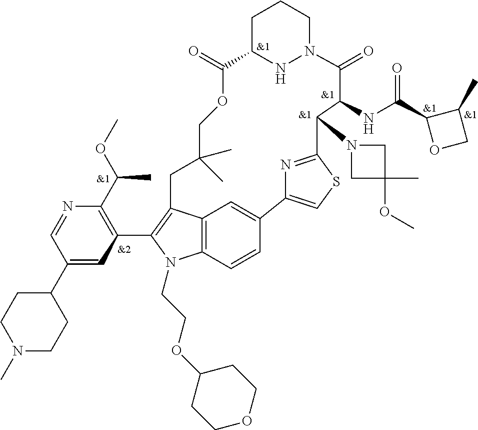 |
| A131 | 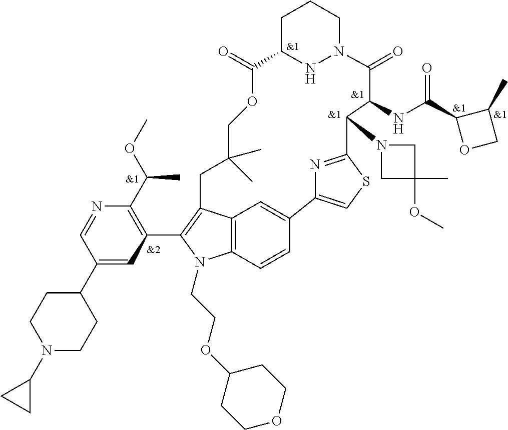 |
| A132 | 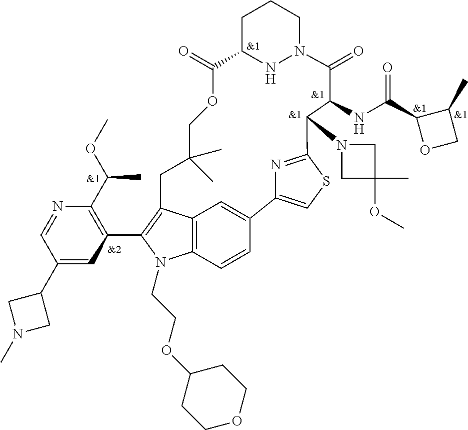 |
| A133 | 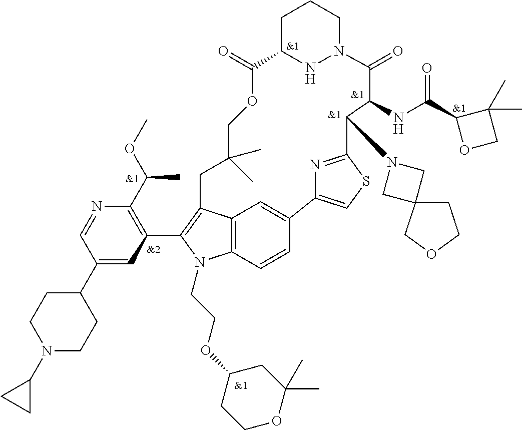 |
| A134 | 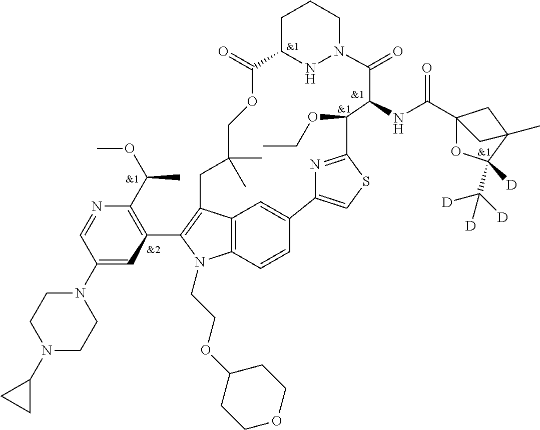 |
| A135 | 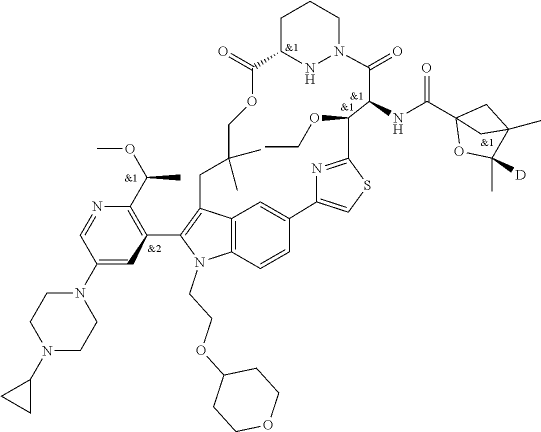 |
| A136 | 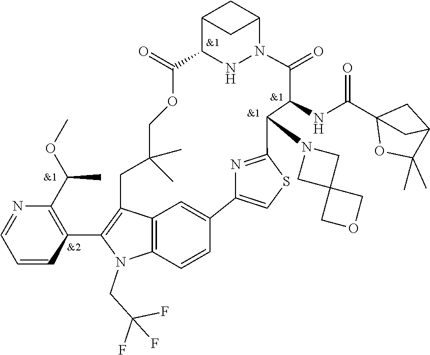 |
| A137 | 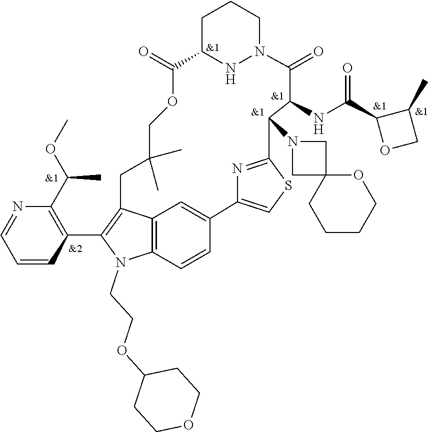 |
| A138 | 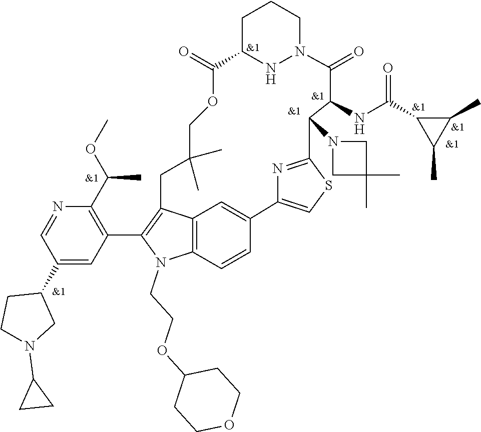 [SMILES: CO[C@@H](C)c1ncc([C@H]2CCN(C3CC3)C2)cc1-c1c2c3cc(ccc3n1CCOC1CCOCC1)-c1csc(n1)[C@@H](N1CC(C)(C)C1)[C@H](NC(=O)[C@H]1[C@H](C)[C@@H]1C)C(=O)N1CCC[C@H](N1)C(=O)OCC(C)(C)C2] |
| A139 | 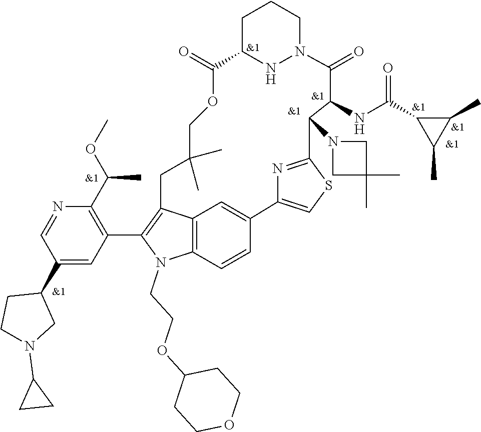 [SMILES: CO[C@@H](C)c1ncc([C@@H]2CCN(C3CC3)C2)cc1-c1c2c3cc(ccc3n1CCOC1CCOCC1)-c1csc(n1)[C@@H](N1CC(C)(C)C1)[C@H](NC(=O)[C@H]1[C@H](C)[C@@H]1C)C(=O)N1CCC[C@H](N1)C(=O)OCC(C)(C)C2] |
| A140 | 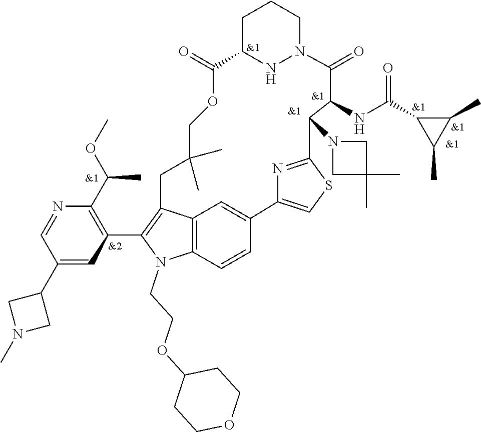 |
| A141 | 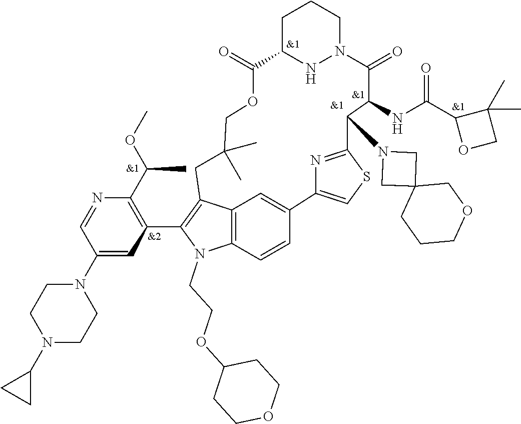 |
| A142 | 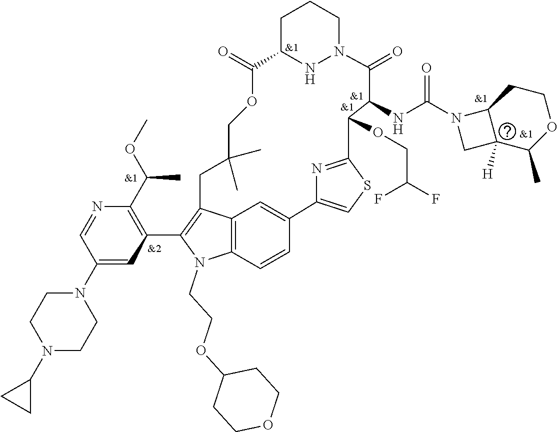 |
| A143 | 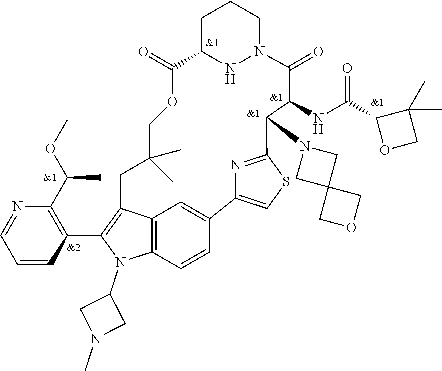 |
| A144 | 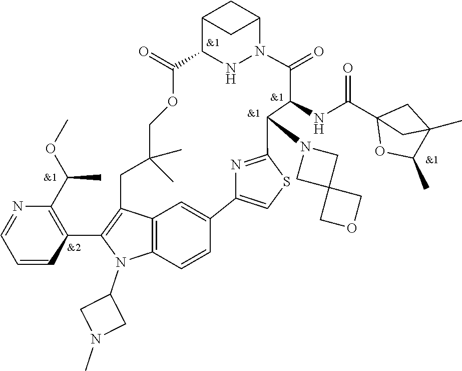 |
| A145 | 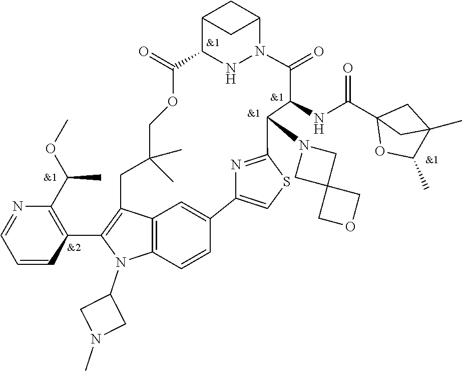 |
| A146 | 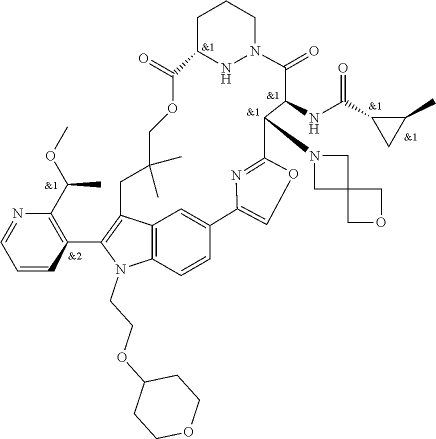 |
| A147 | 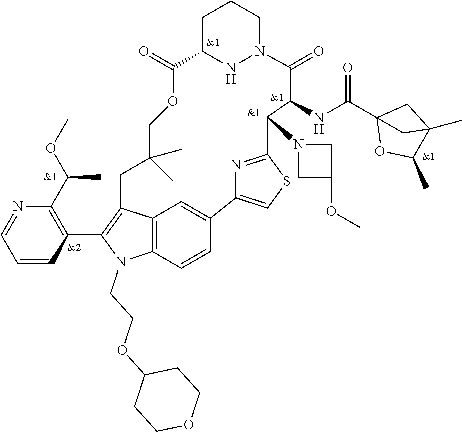 |
| A148 | 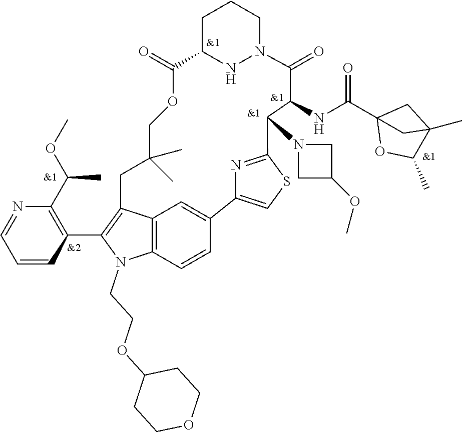 |
| A149 | 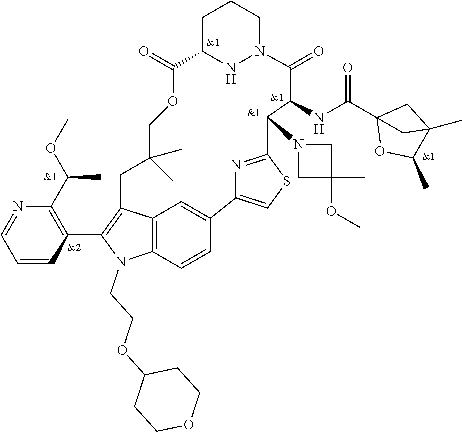 |
| A150 | 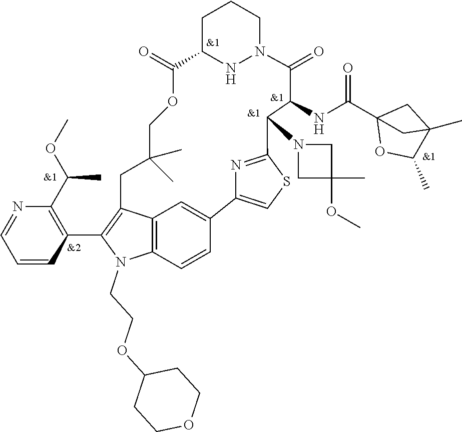 |
| A151 | 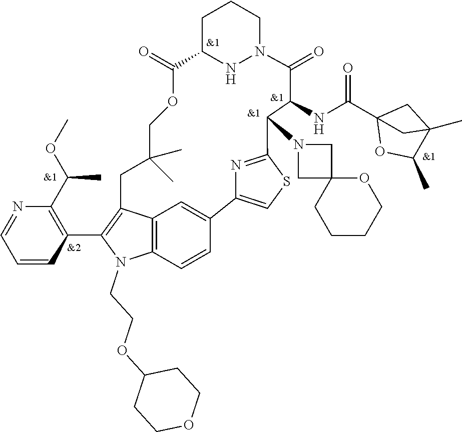 |
| A152 | 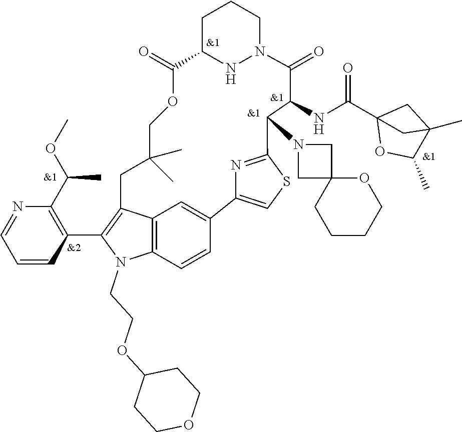 |
| A153 | 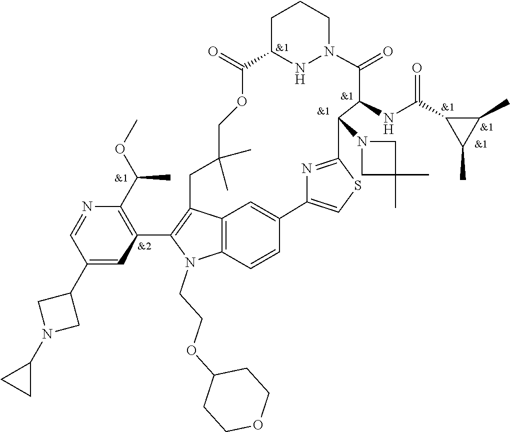 |
| A154 | 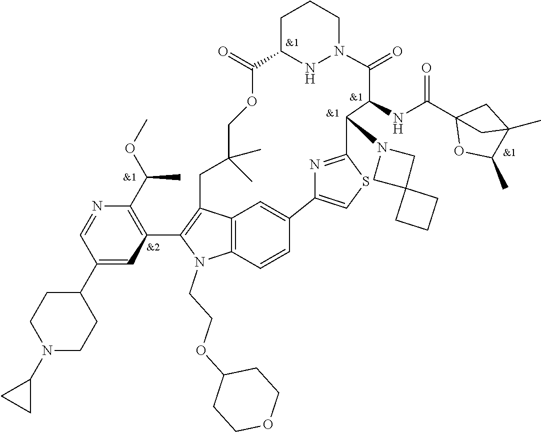 |
| A155 | 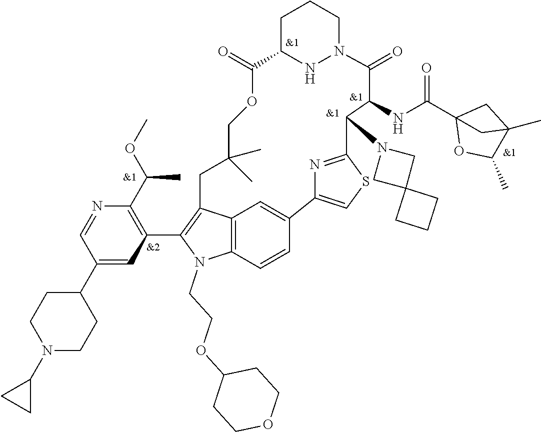 |
| A156 | 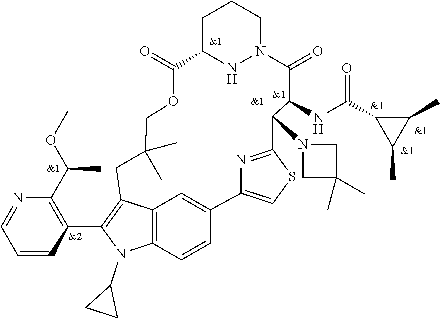 |
| A157 | 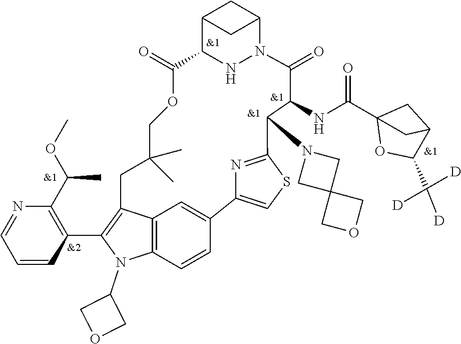 |
| A158 | 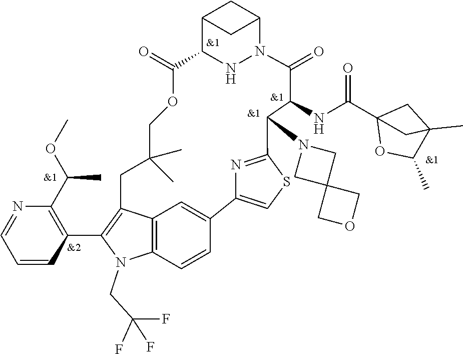 |
| A159 | 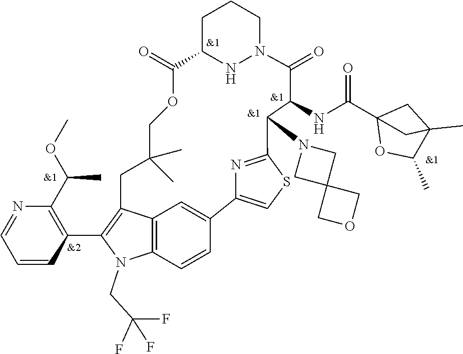 |
| A160 | 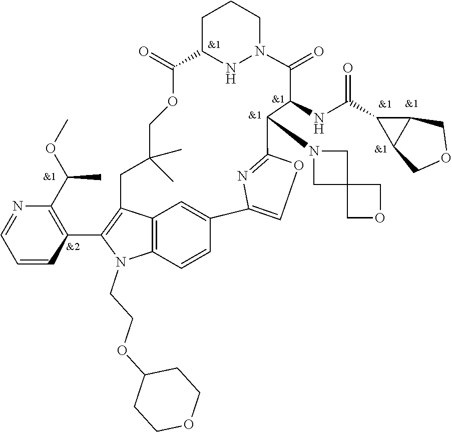 |
| A161 | 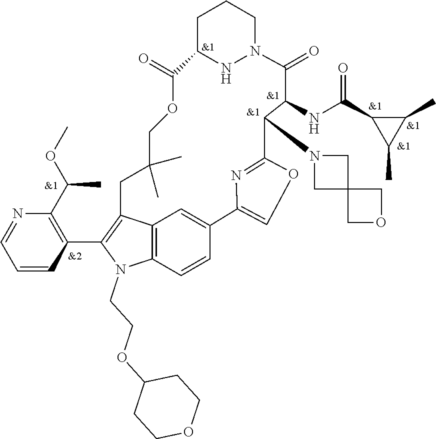 |
| A162 | 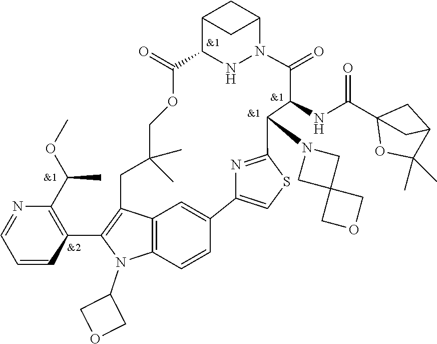 |
| A163 | 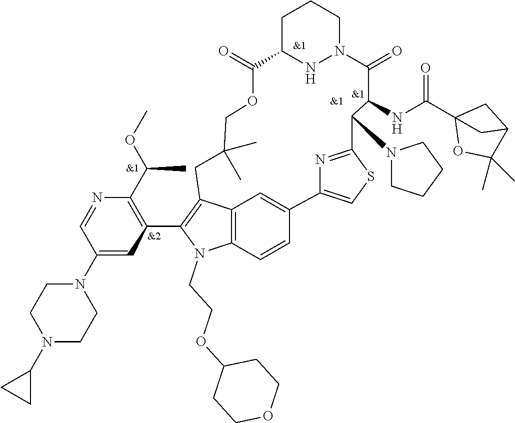 |
| A164 | 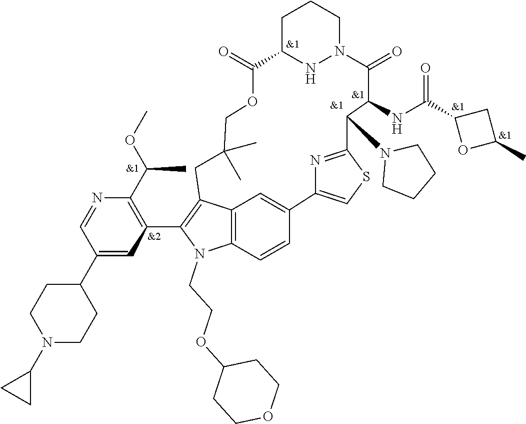 |
| A165 | 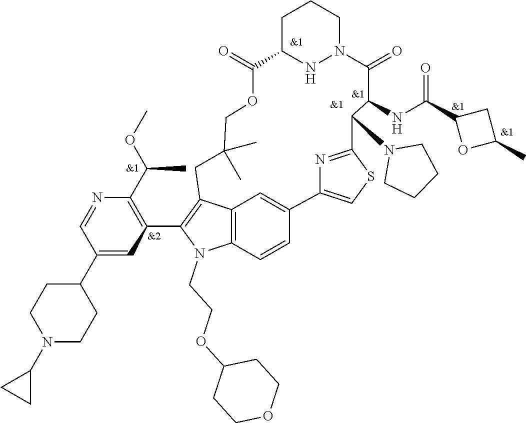 |
| A166 | 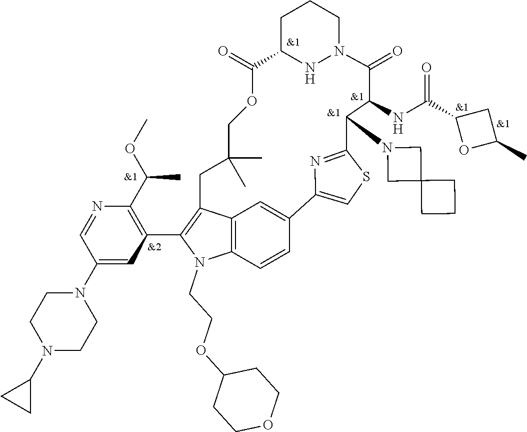 |
| A167 | 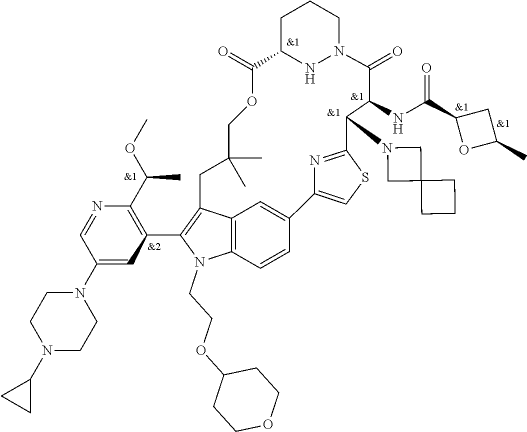 |
| A168 | 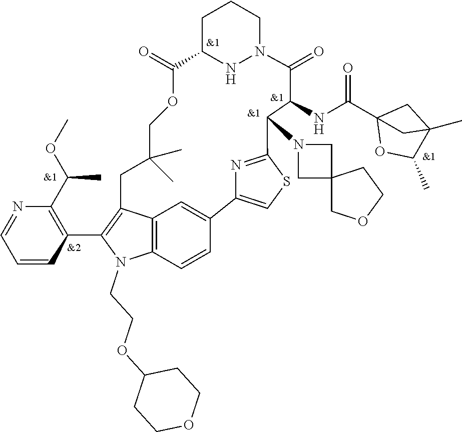 |
| A169 | 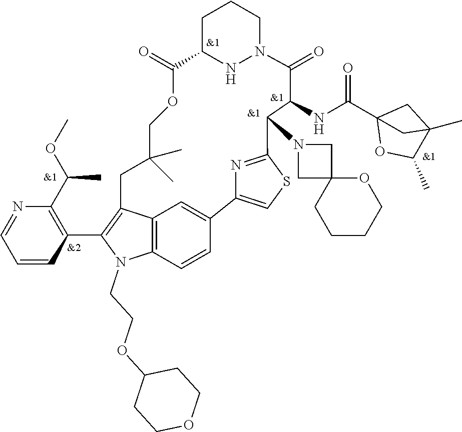 |
| A170 | 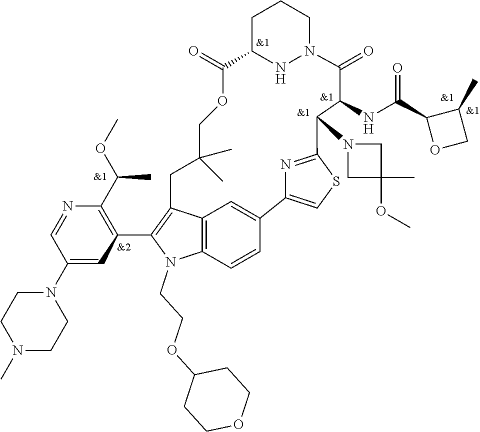 |
| A171 | 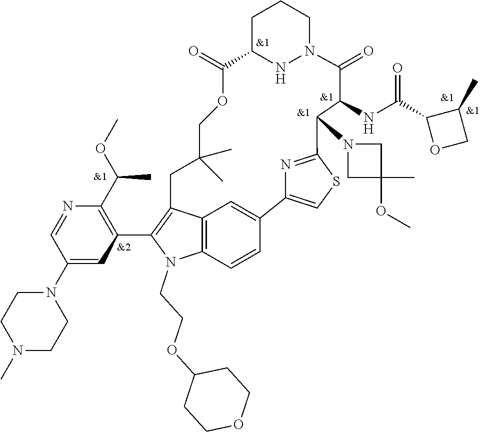 |
| A172 | 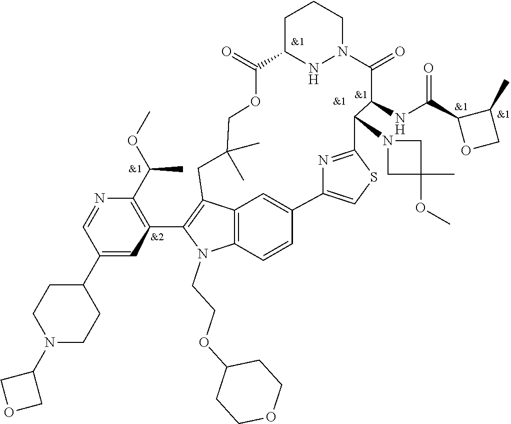 |
| A173 | 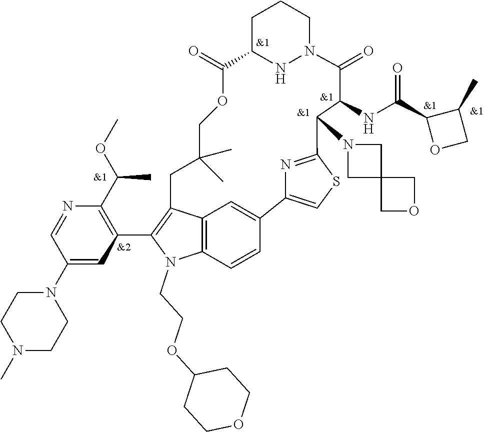 |
| A174 | 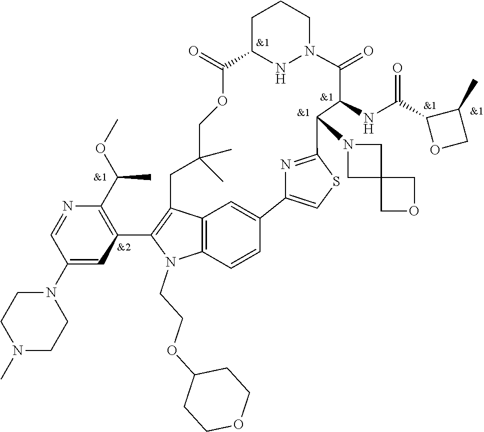 |
| A175 | 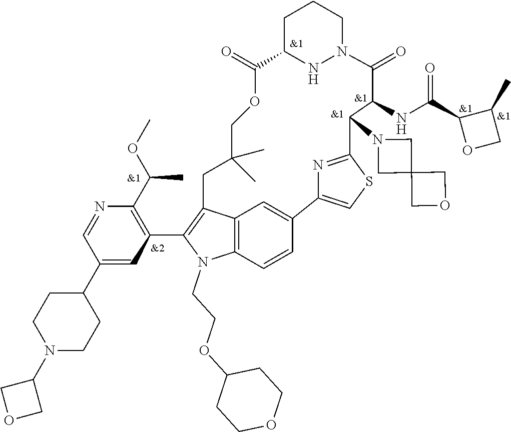 |
| A176 | 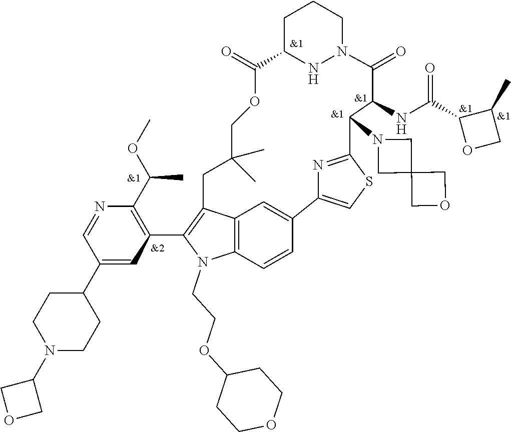 |
| A177 | 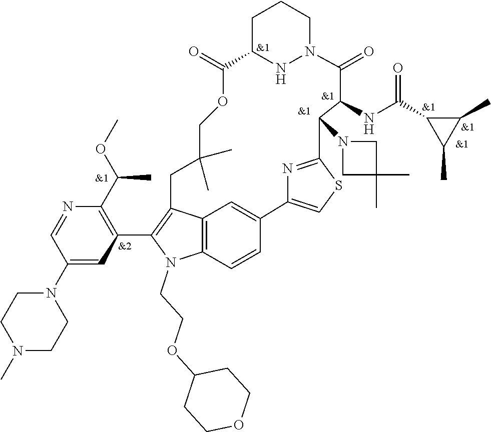 |
| A178 | 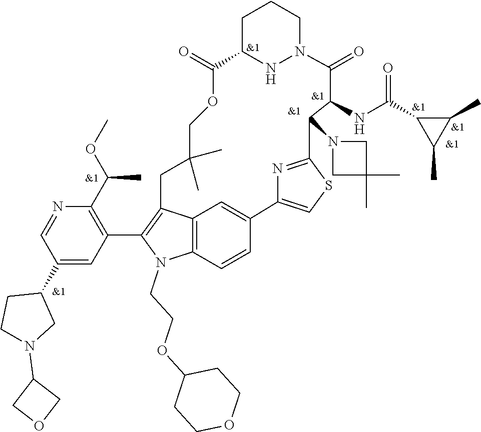 |
| A179 | 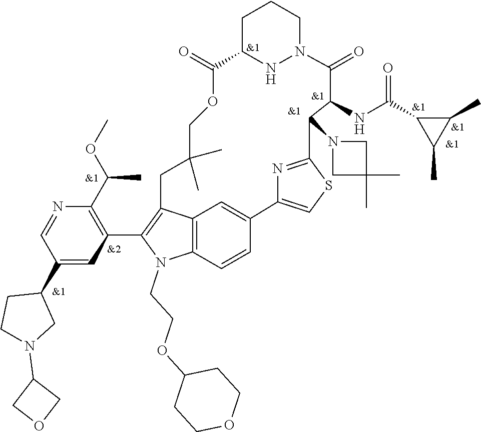 |
| A180 | 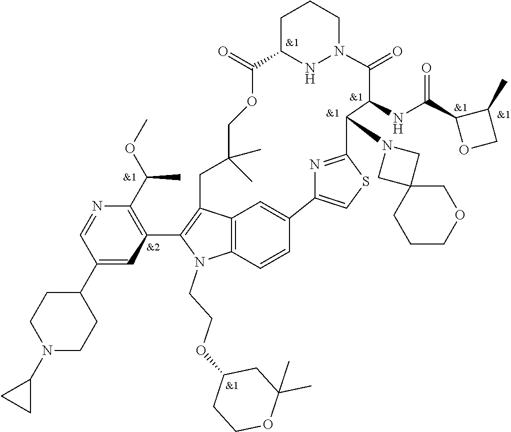 |
| A181 | 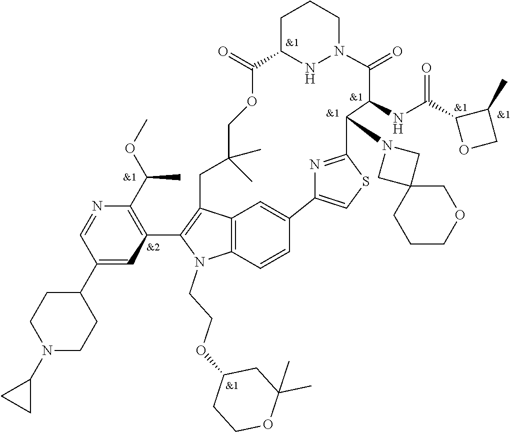 |
| A182 | 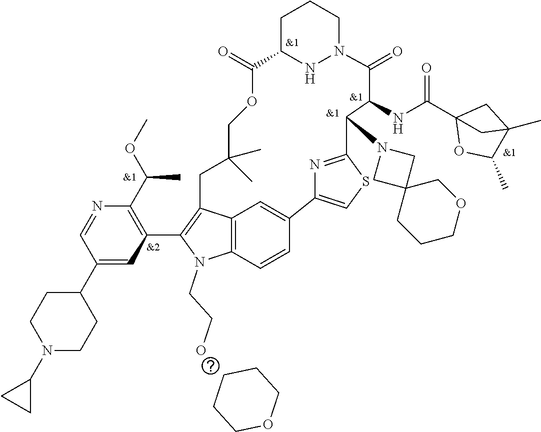 |
| A183 | 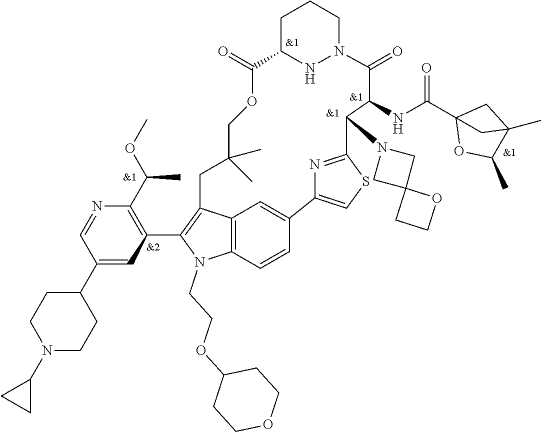 |
| A184 | 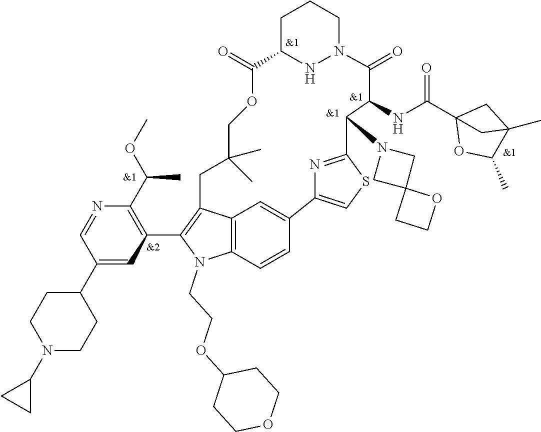 |
| A185 | 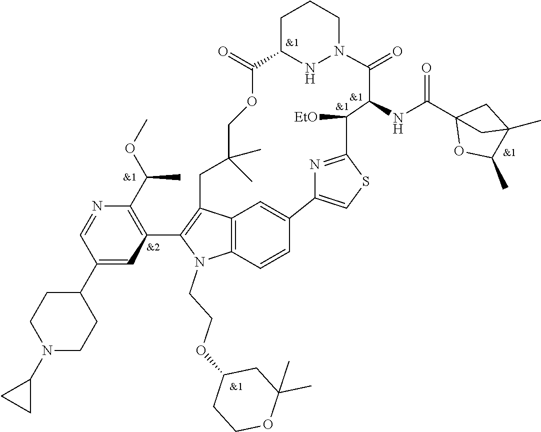 |
| A186 | 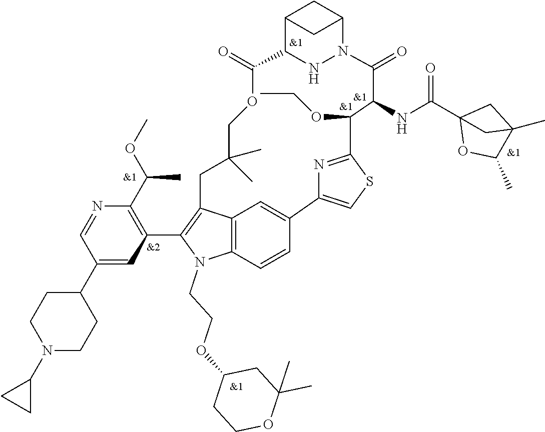 |
| A187 | 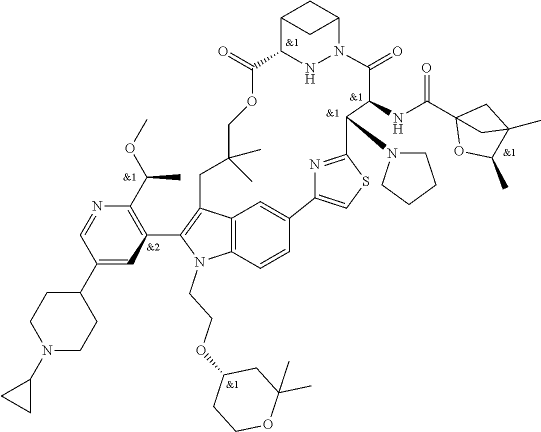 |
| A188 | 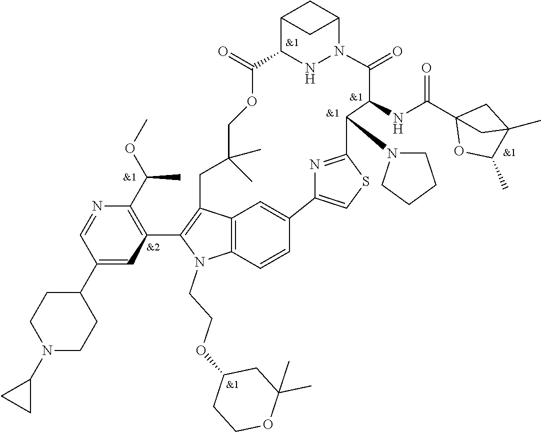 |
| A189 | 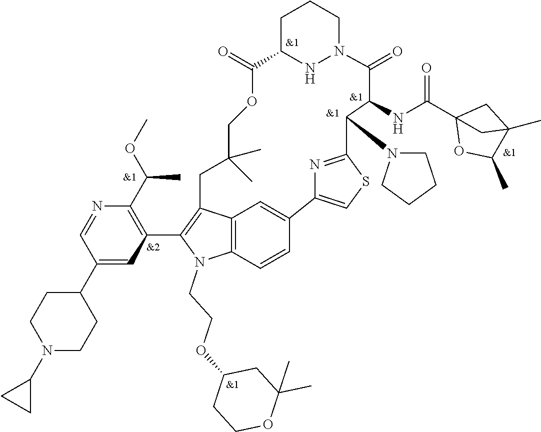 |
| A190 | 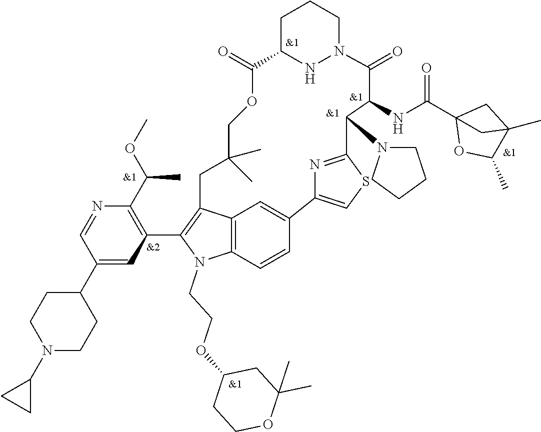 |
| A191 | 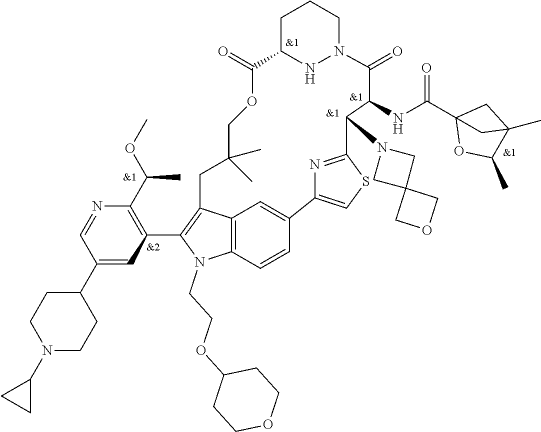 |
| A192 | 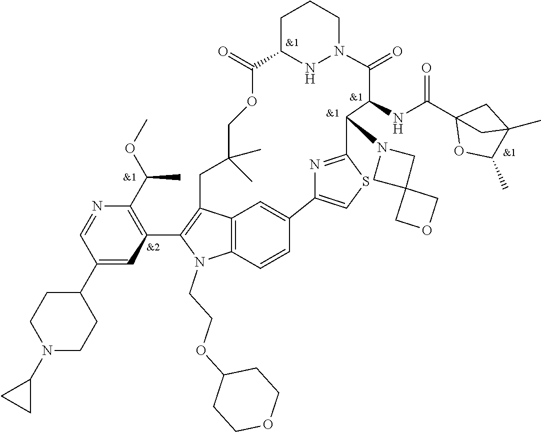 |
| A193 | 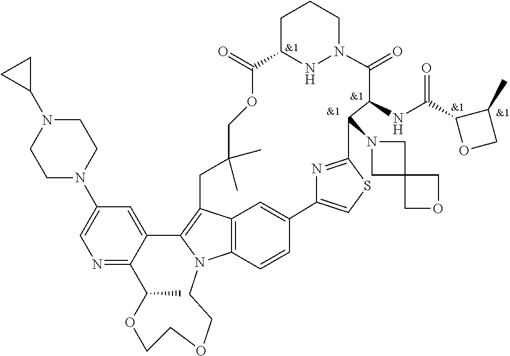 |
| A194 | 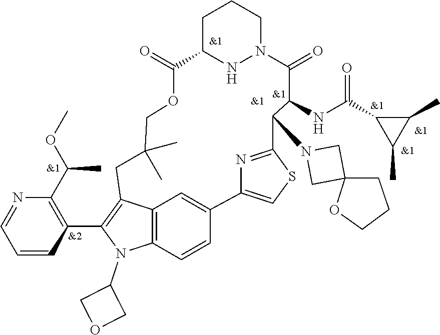 |
| A195 | 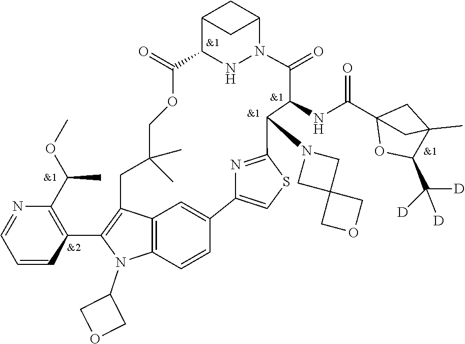 |
| A196 | 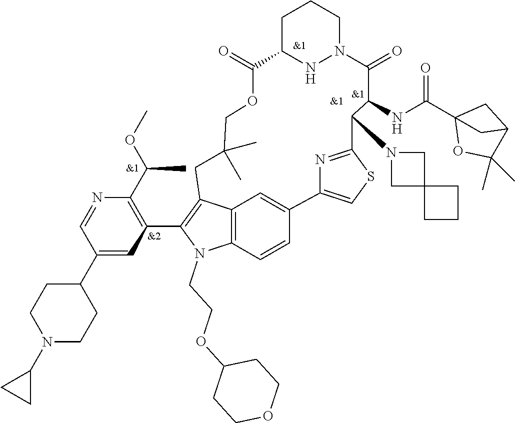 |
| A197 | 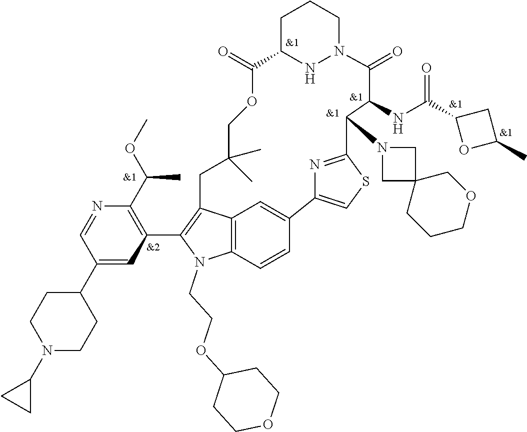 |
| A198 | 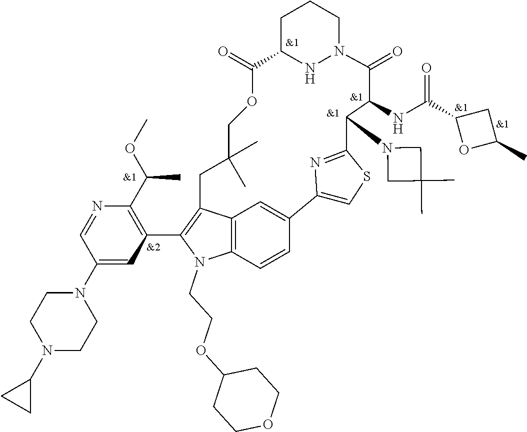 |
| A199 | 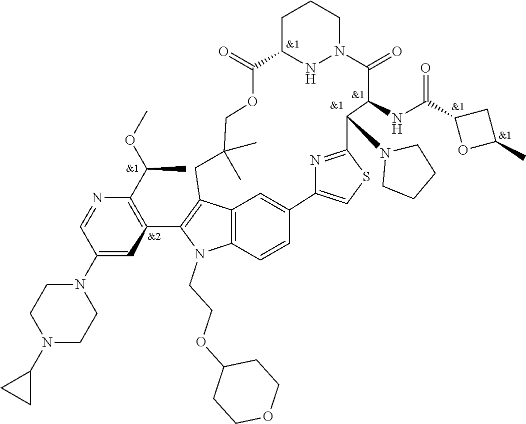 |
| A200 | 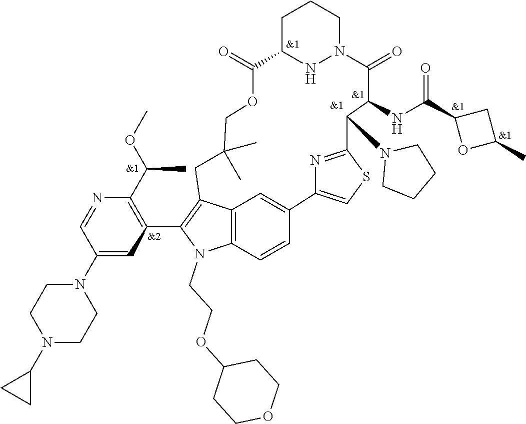 |
| A201 | 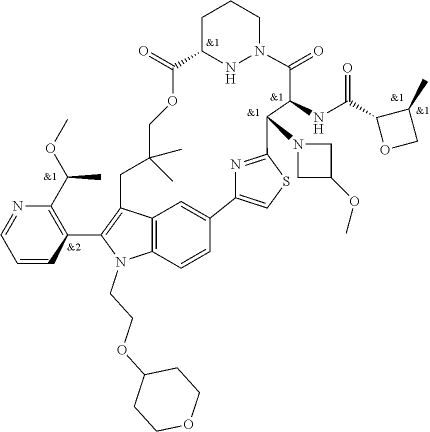 |
| A202 | 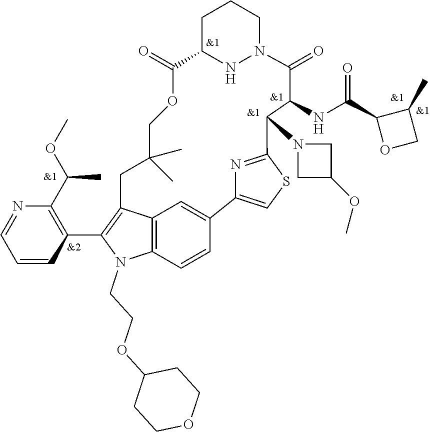 |
| A203 | 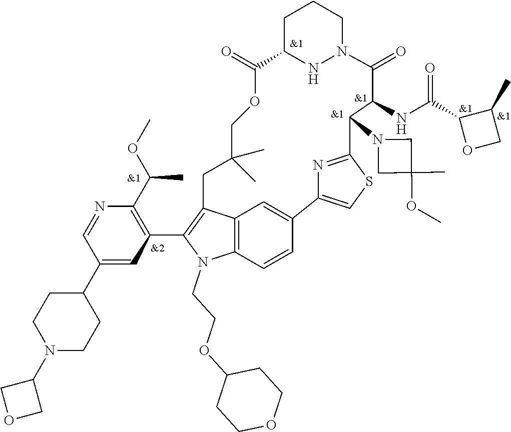 |
| A204 | 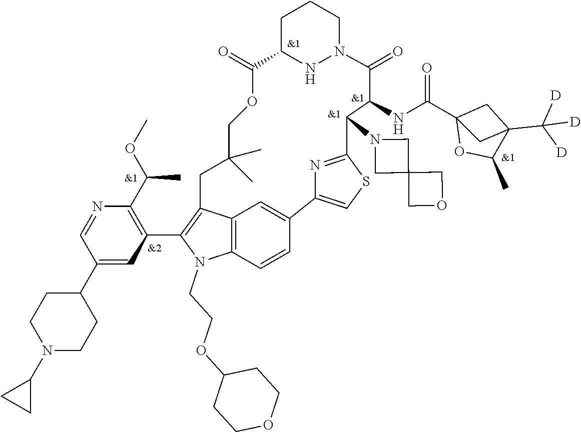 |
| A205 | 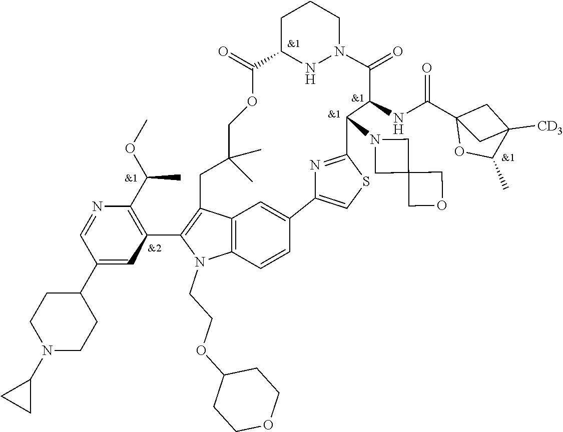 |
| A206 | 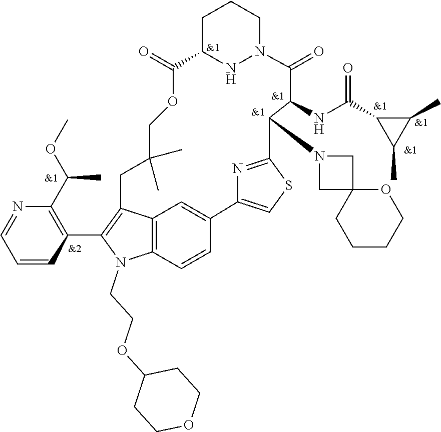 |
| A207 | 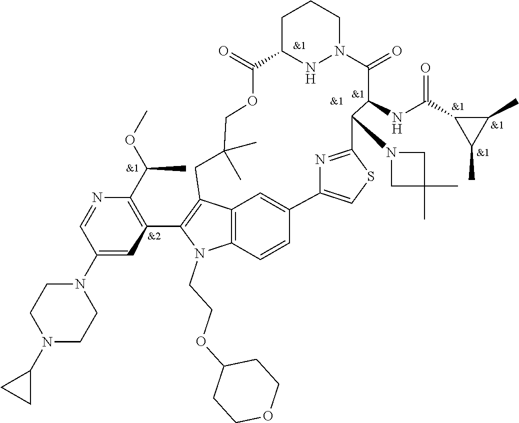 |
| A208 | 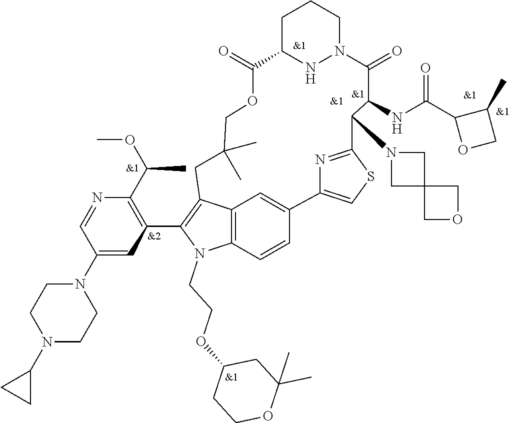 |
| A209 | 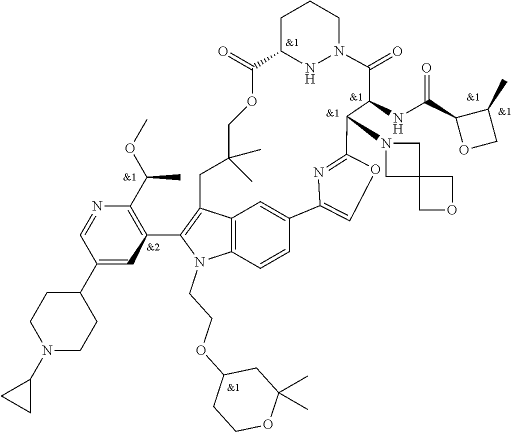 |
| A210 | 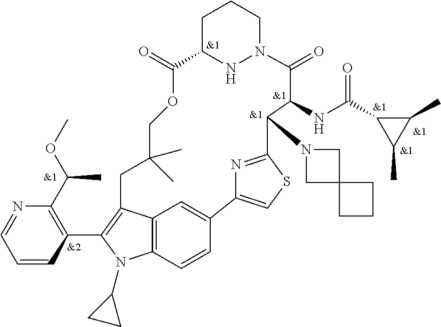 |
| A211 | 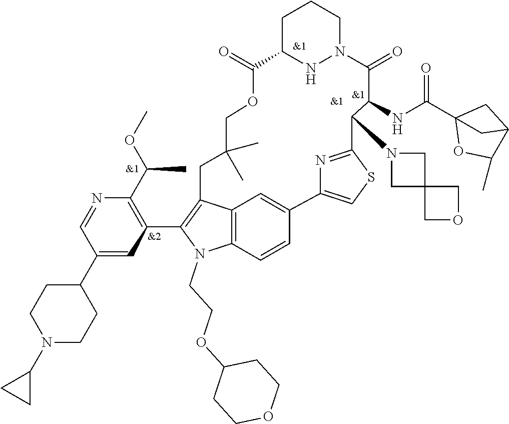 |
| A212 | 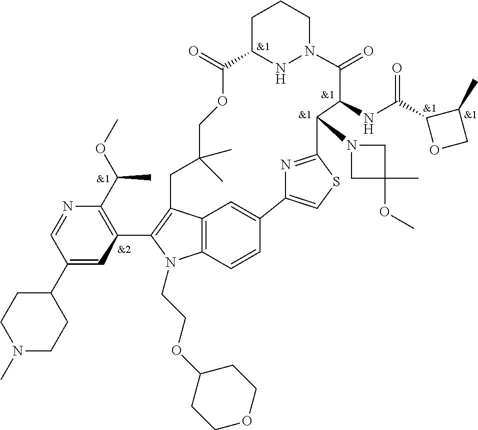 |
| A213 | 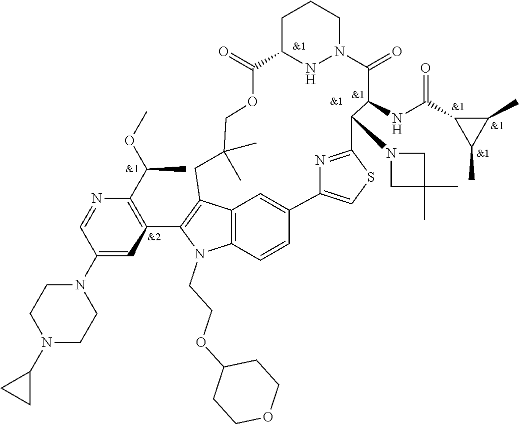 |
| A214 | 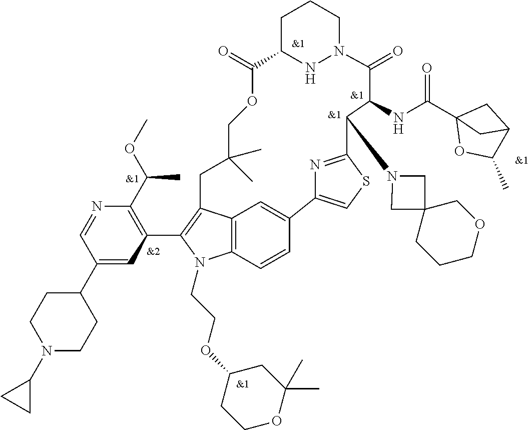 |
| A215 | 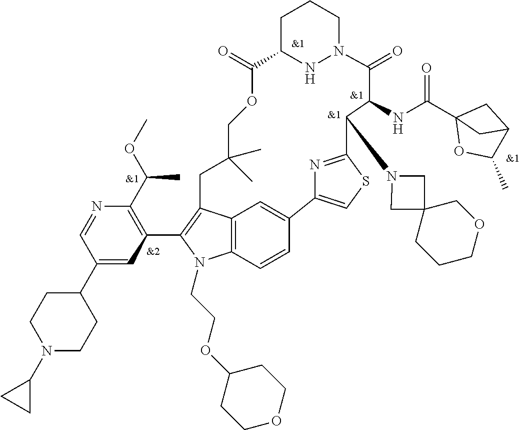 |
| A216 | 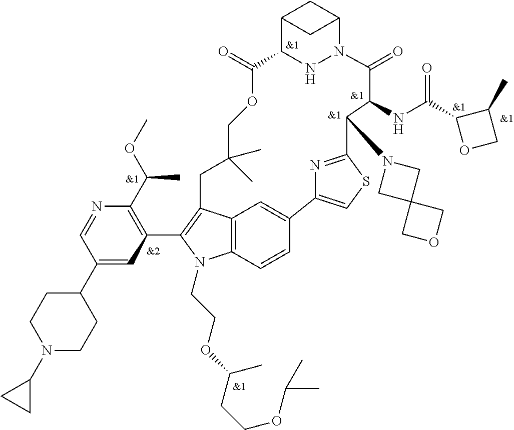 |
| A217 | 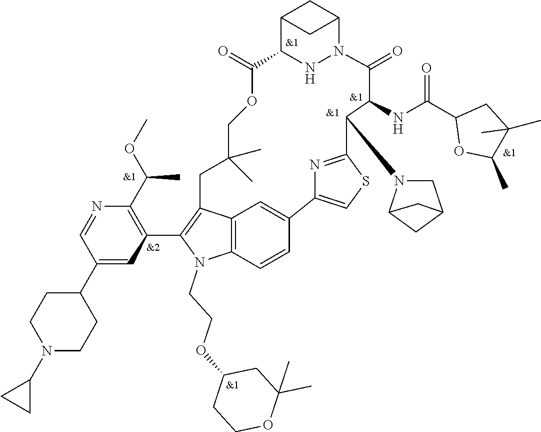 |
| A218 | 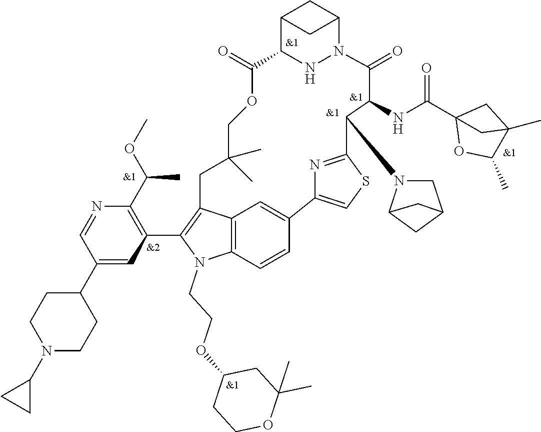 |
| A219 | 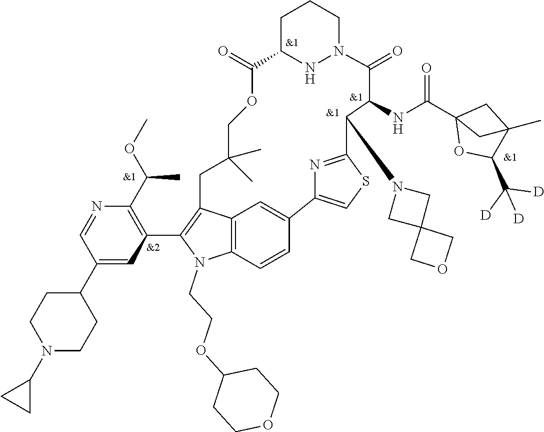 |
| A220 | 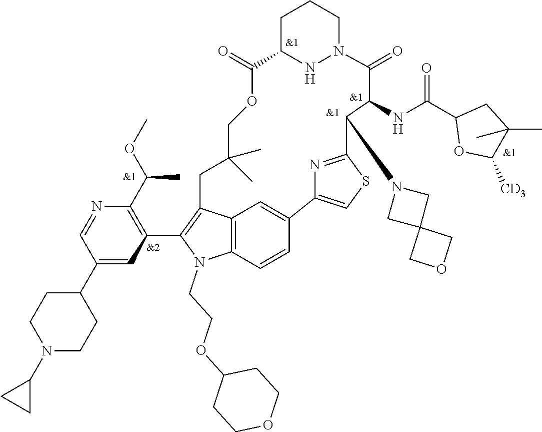 |
| A221 | 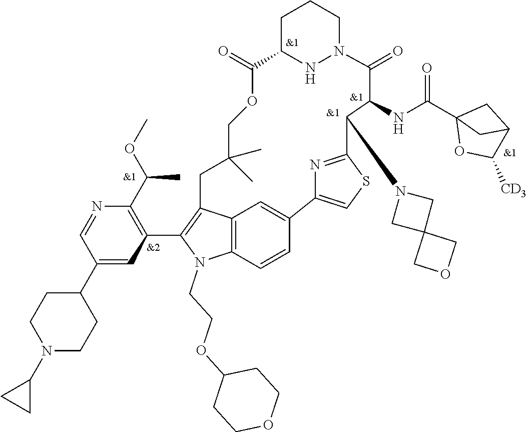 |
| A222 | 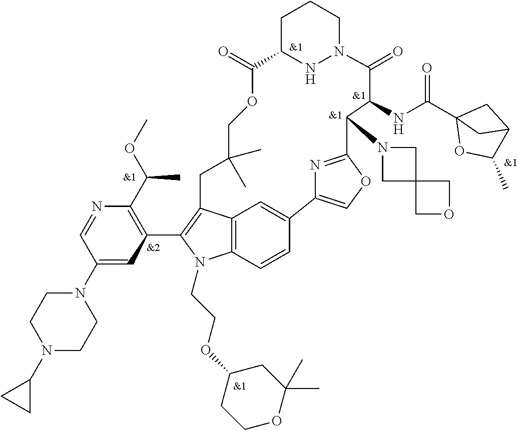 |
| A223 | 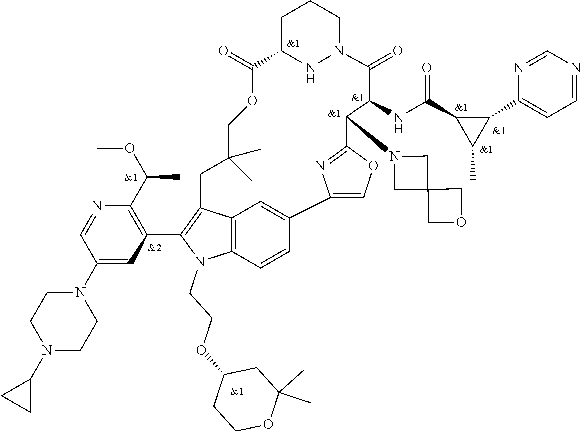 |
| A224 | 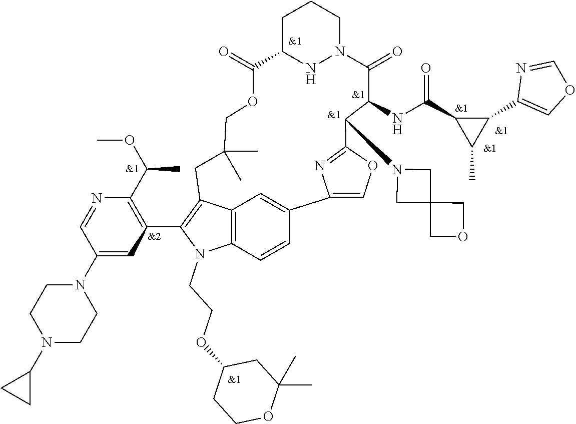 |
| A225 | 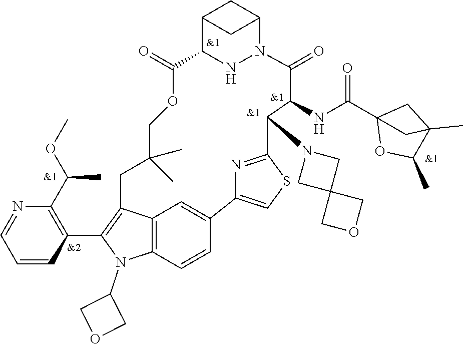 |
| A226 | 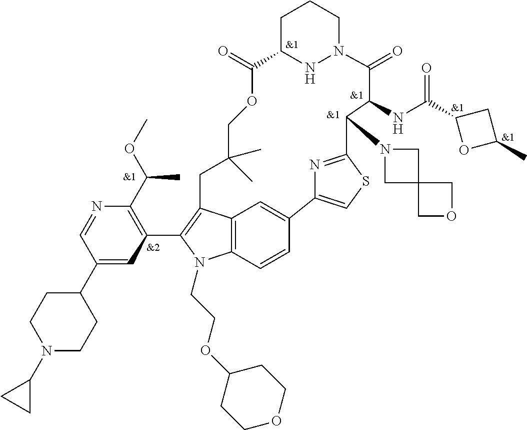 |
| A227 | 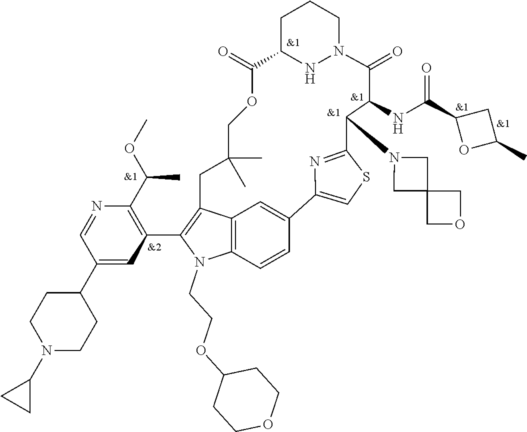 |
| A228 | 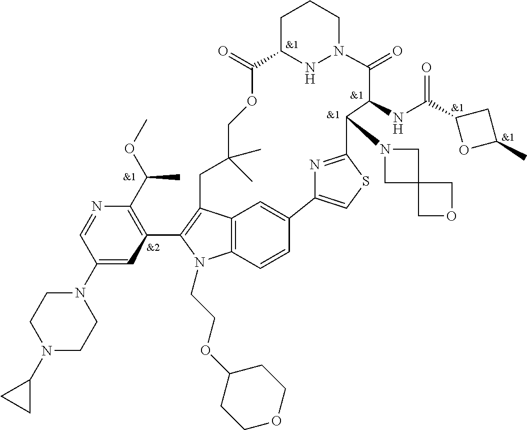 |
| A229 | 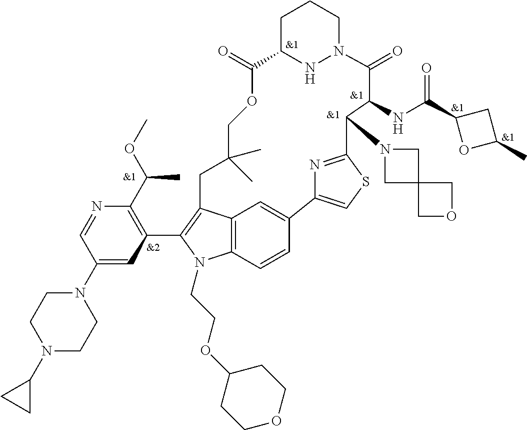 |
| A230 | 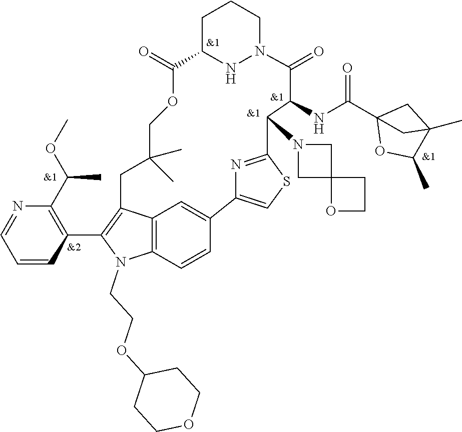 |
| A231 | 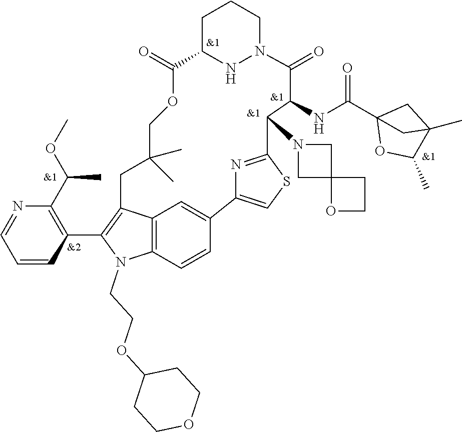 |
| A232 | 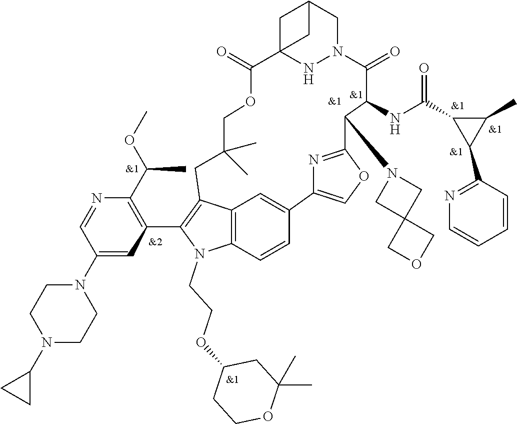 |
| A233 | 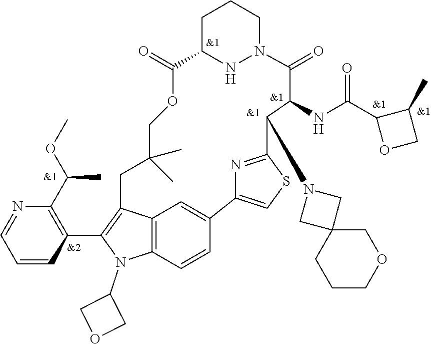 |
| A234 | 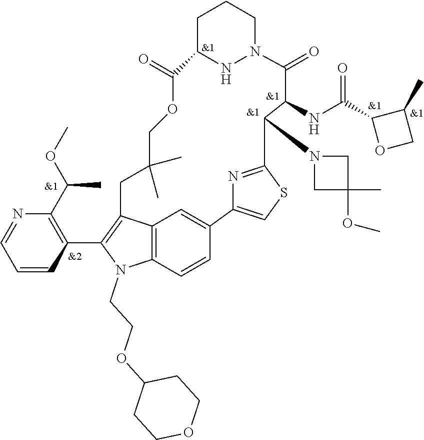 |
| A235 | 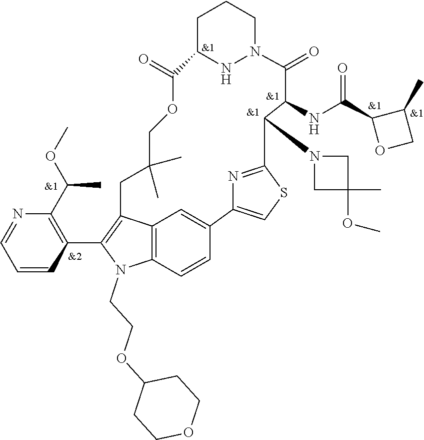 |
| A236 | 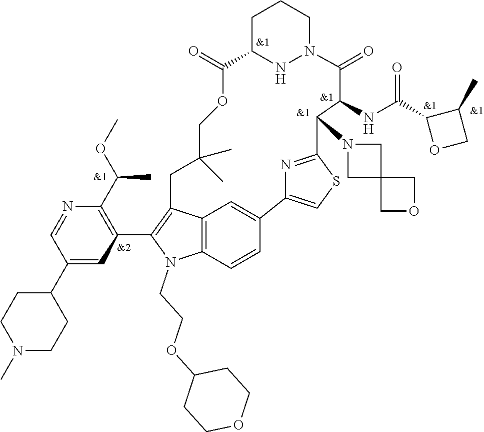 |
| A237 | 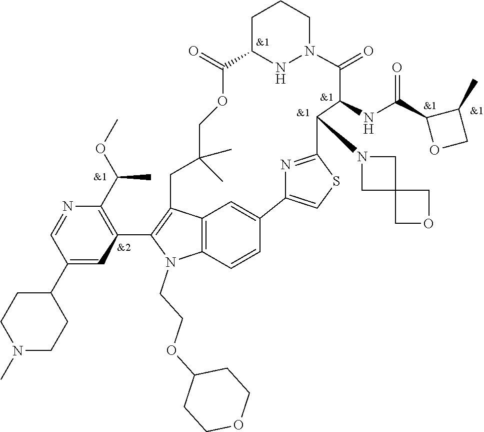 |
| A238 | 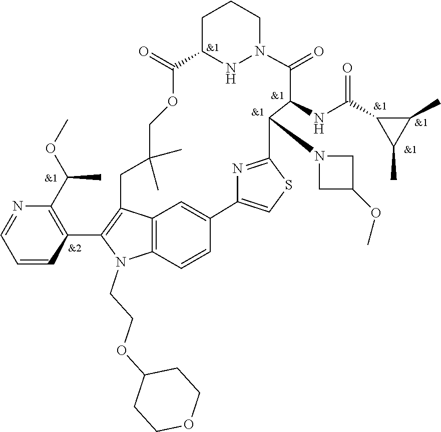 |
| A239 | 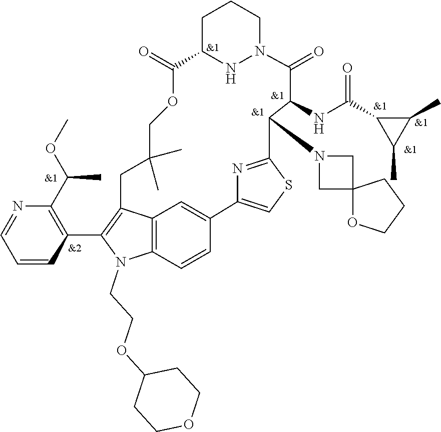 |
| A240 | 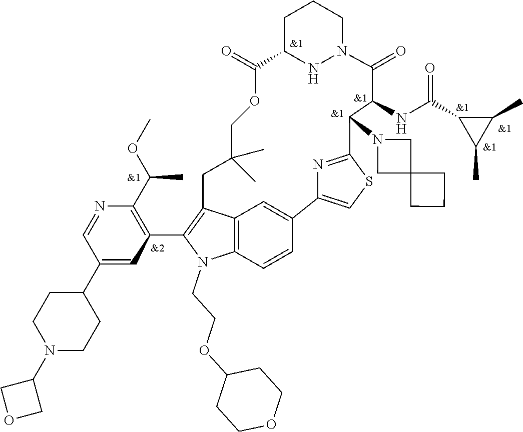 |
| A241 | 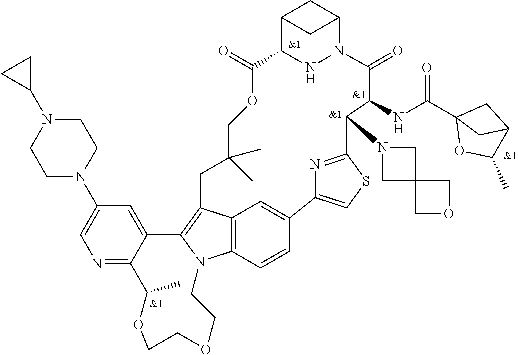 [SMILES: C[C@@H]1OCCOCCn2c(c3c4cc(ccc42)-c2csc(n2)[C@@H](N2CC4(COC4)C2)[C@H](NC(=O)C24CC(C2)[C@H](C)O4)C(=O)N2N[C@H](C(=O)OCC(C)(C)C3)C3CC2C3)-c2cc(N3CCN(C4CC4)CC3)cnc21] |
| A242 | 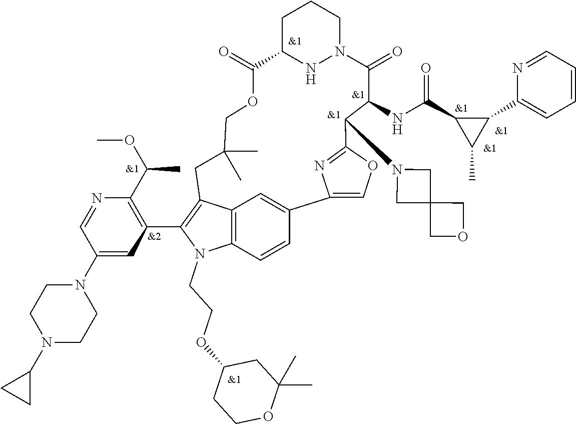 |
| A243 | 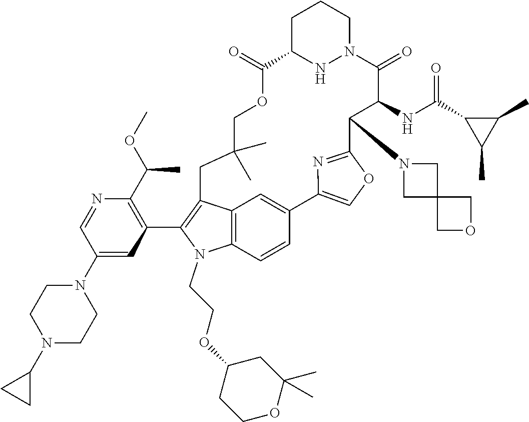 |
| A244 | 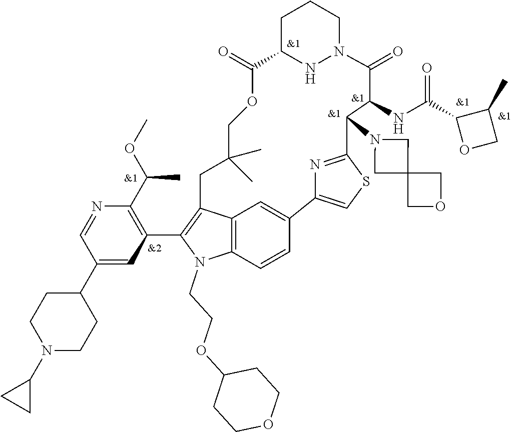 |
| A245 | 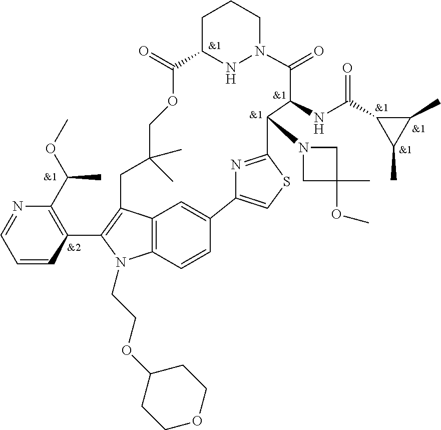 |
| A246 | 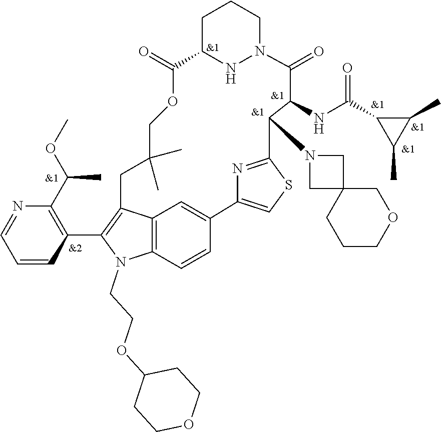 |
| A247 | 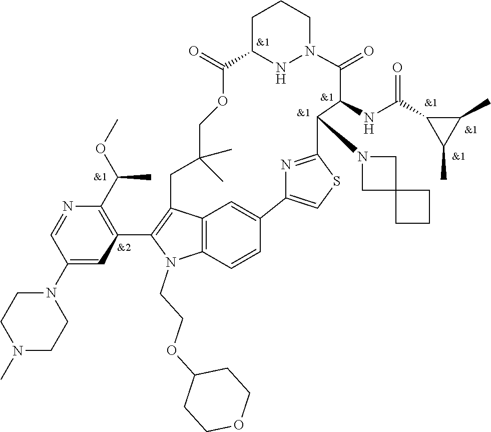 |
| A248 | 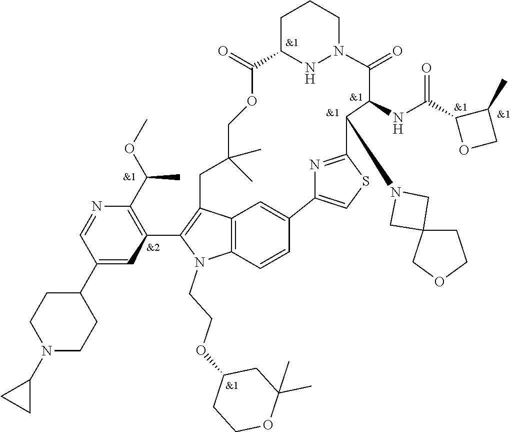 |
| A249 | 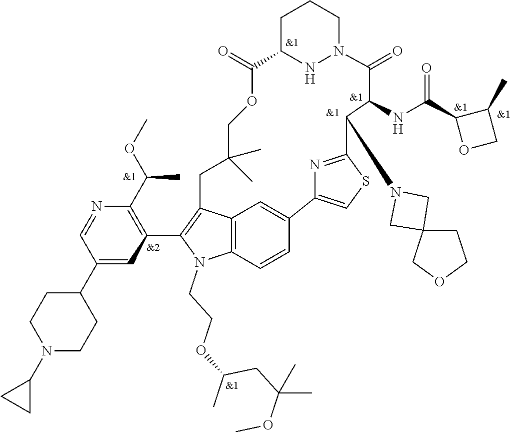 |
| A250 | 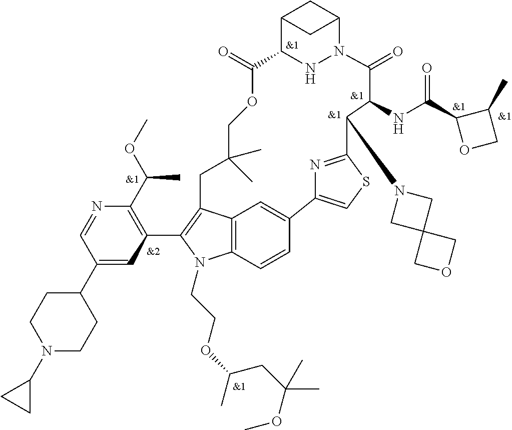 |
| A251 | 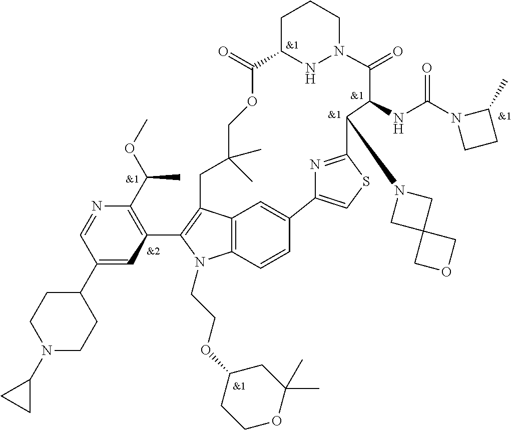 |
| A252 | 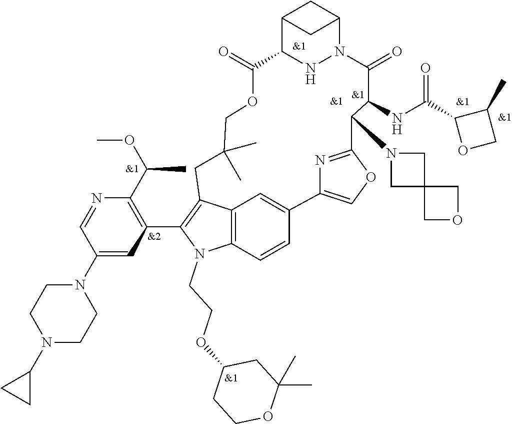 |
| A253 | 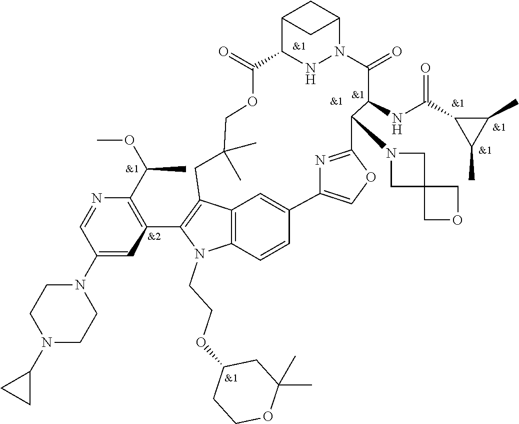 |
| A254 | 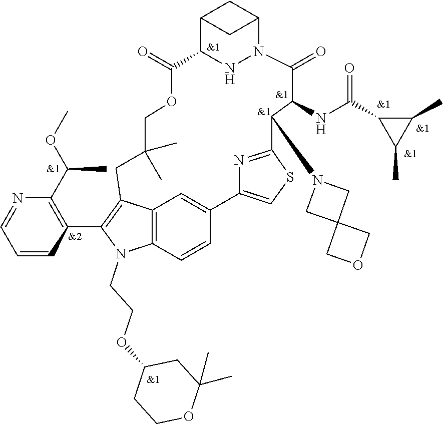 |
| A255 | 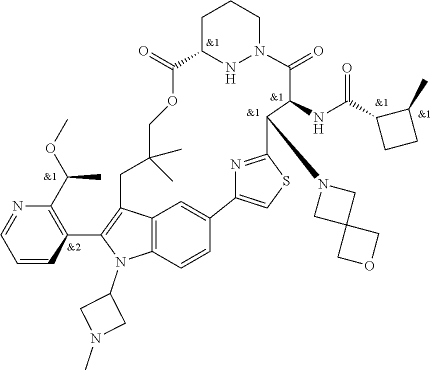 |
| A256 | 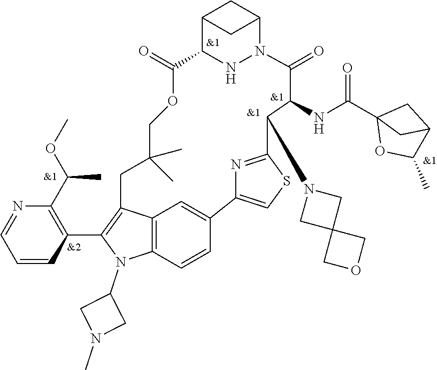 |
| A257 | 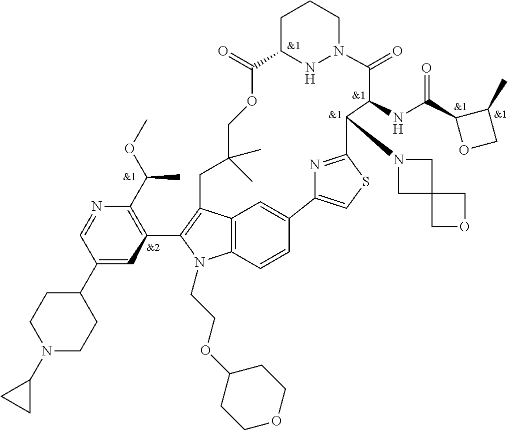 |
| A258 | 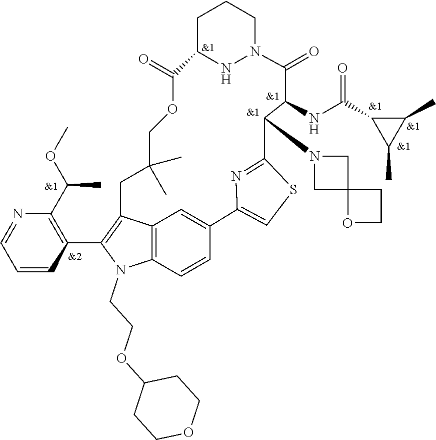 |
| A259 | 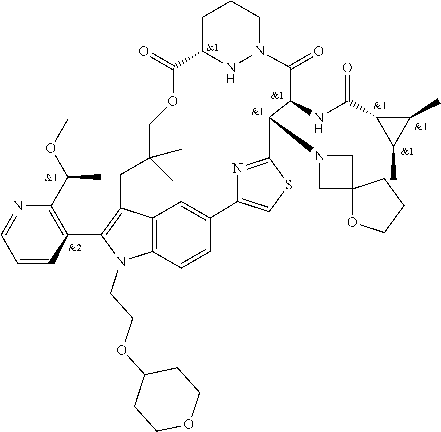 |
| A260 | 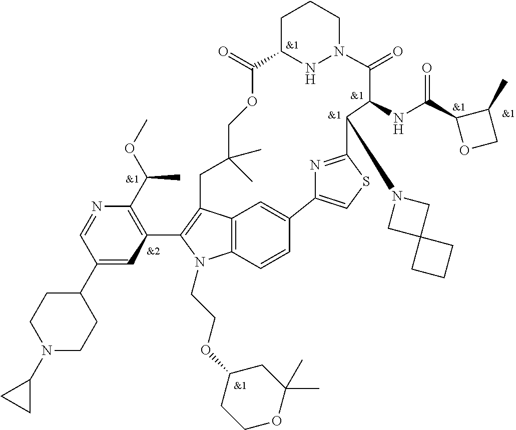 |
| A261 | 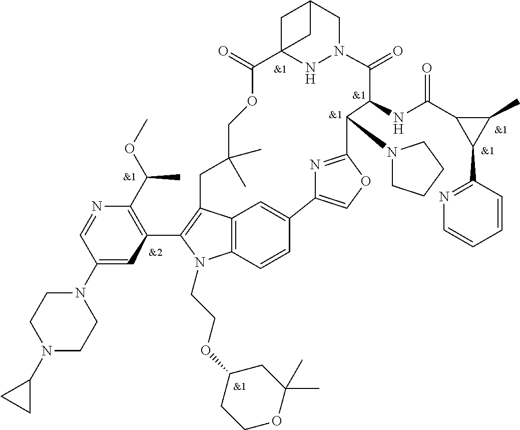 |
| A262 | 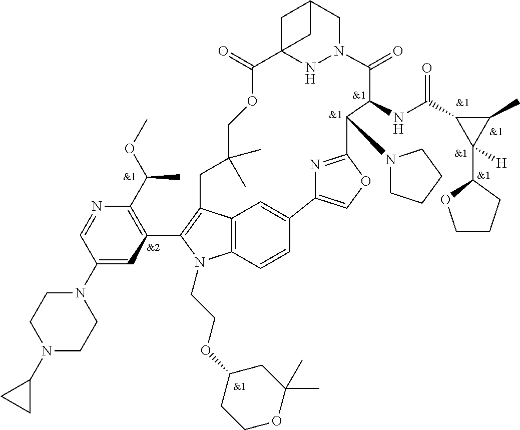 |
| A263 | 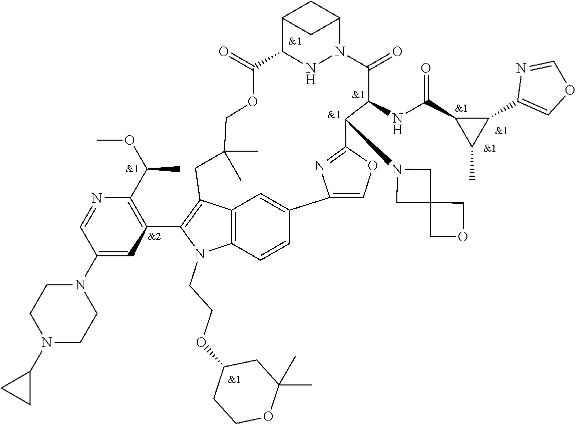 |
| A264 | 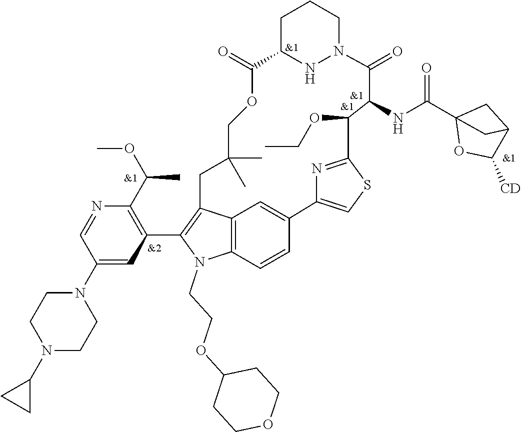 |
| A265 | 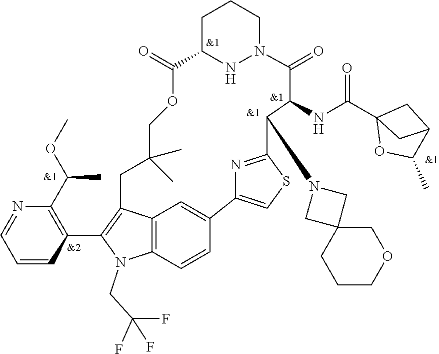 |
| A266 | 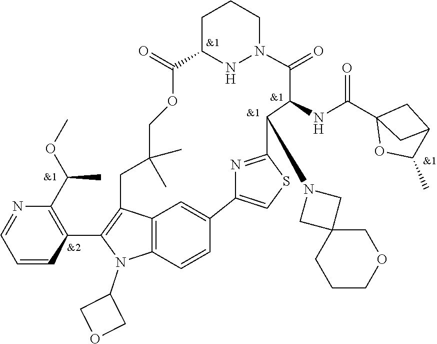 |
| A267 | 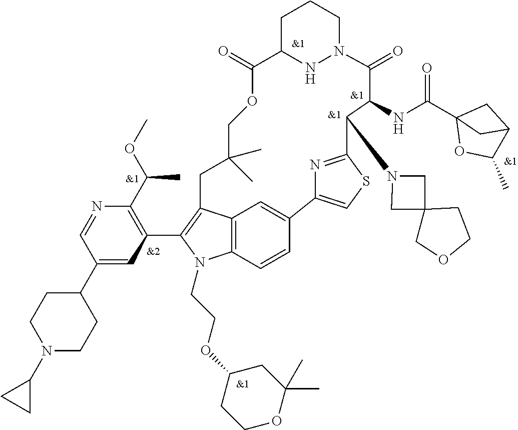 |
| A268 | 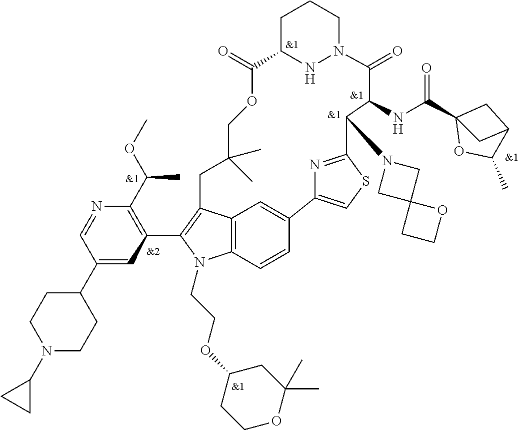 |
| A269 | 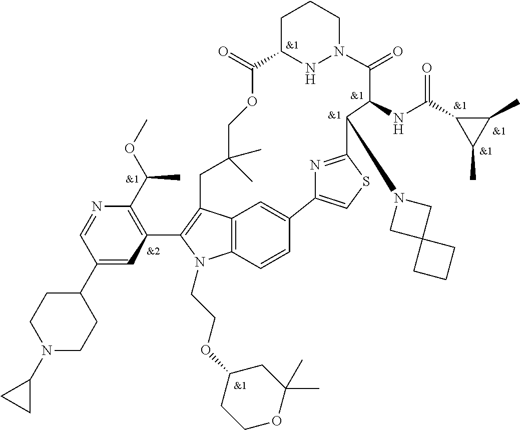 |
| A270 | 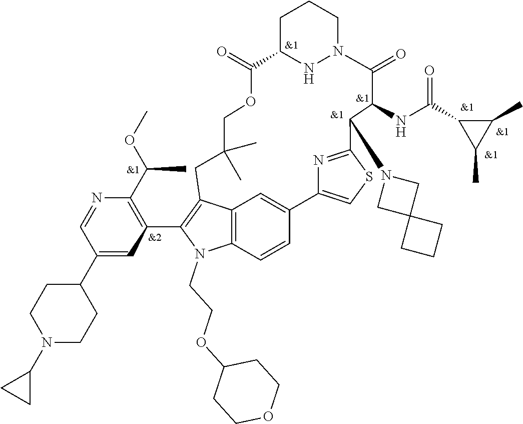 |
| A271 | 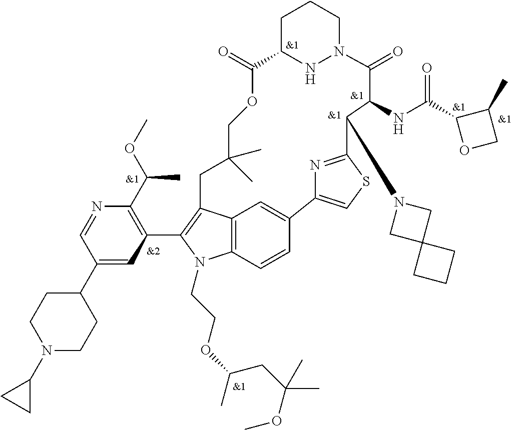 |
| A272 | 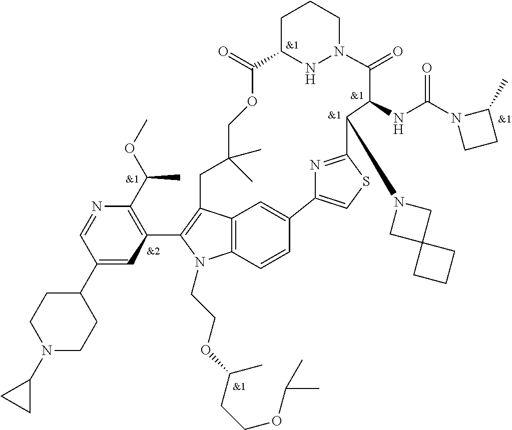 |
| A273 | 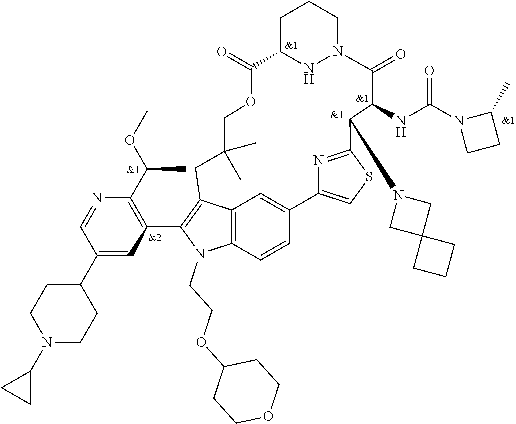 |
| A274 | 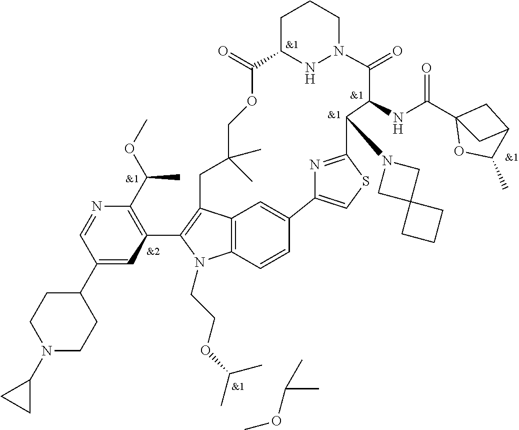 |
| A275 | 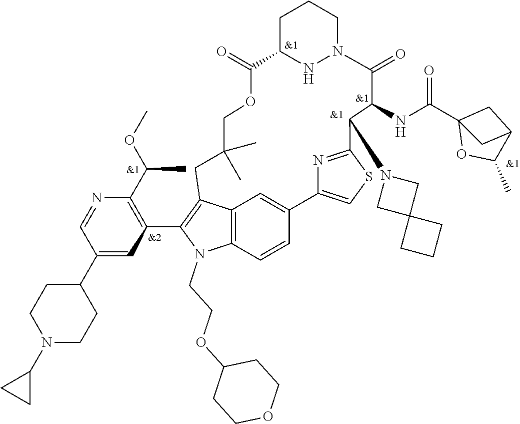 |
| A276 | 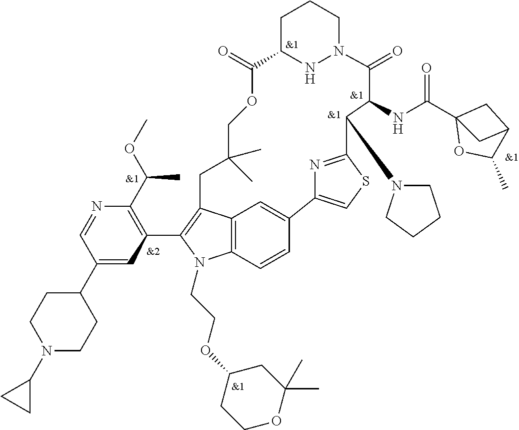 |
| A277 | 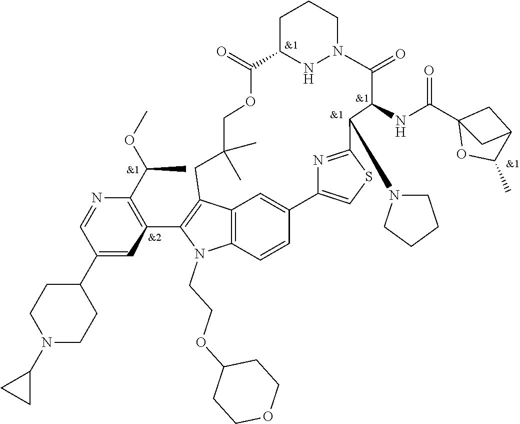 |
| A278 | 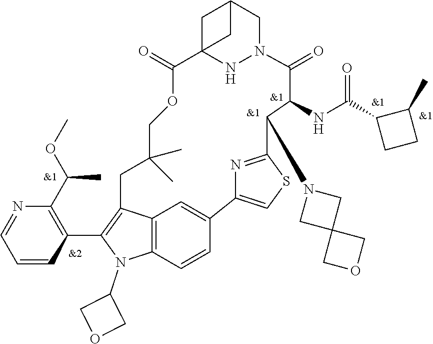 |
| A279 | 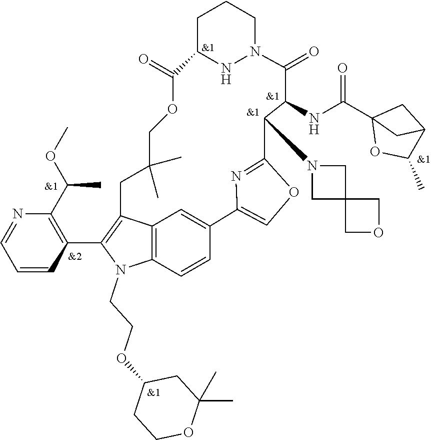 |
| A280 | 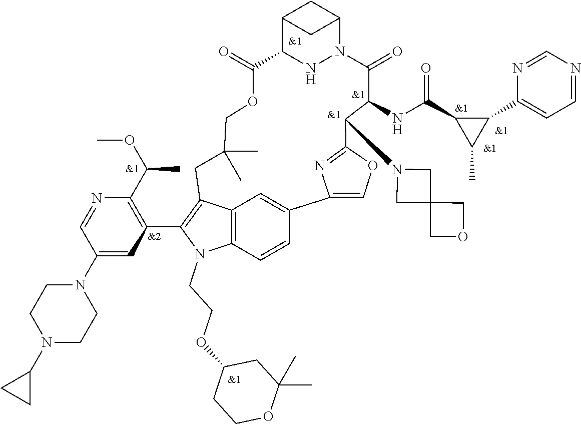 |
| A281 | 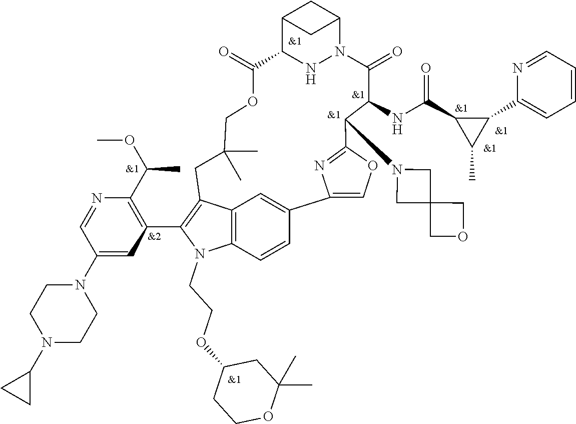 |
| A282 | 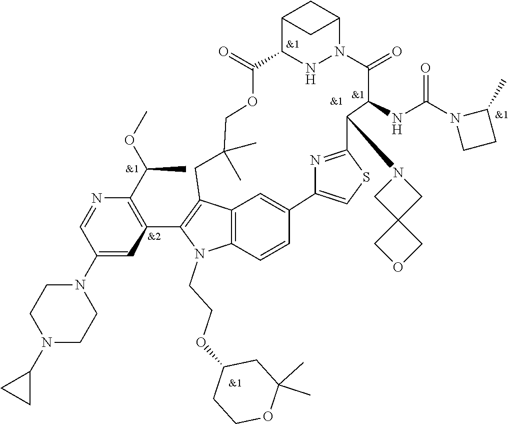 |
| A283 | 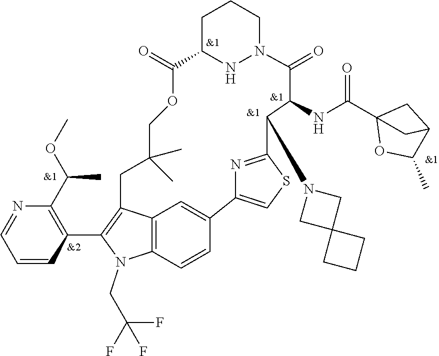 |
| A284 | 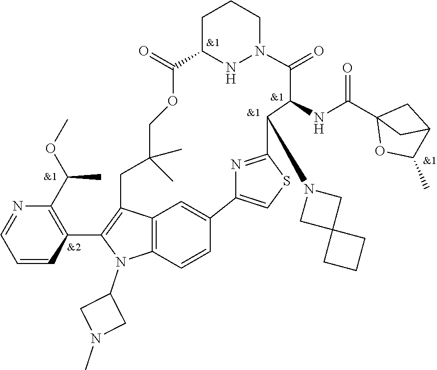 |
| A285 | 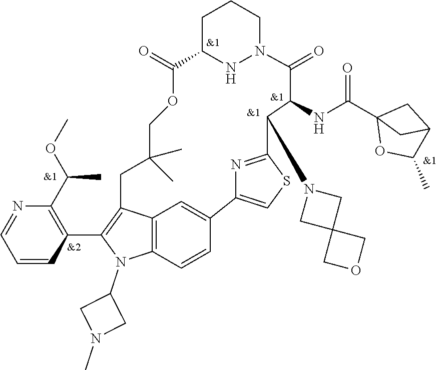 |
| A286 | 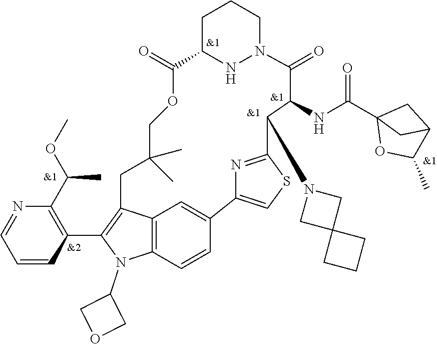 |
| A287 | 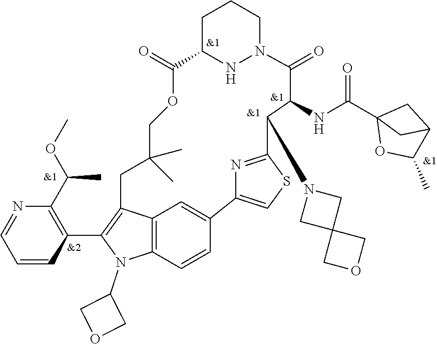 |
| A288 | 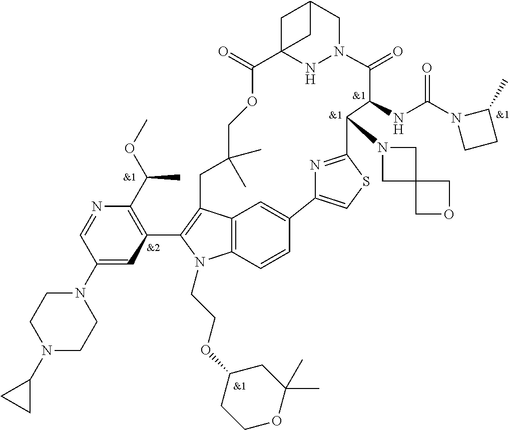 |
| A289 | 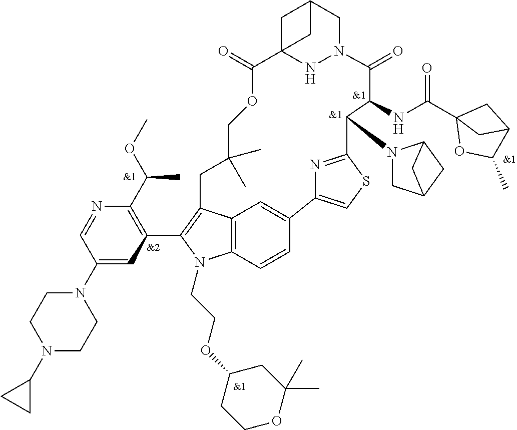 |
| A290 | 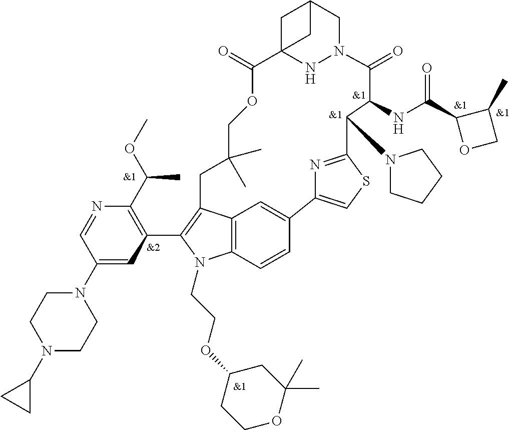 |
| A291 | 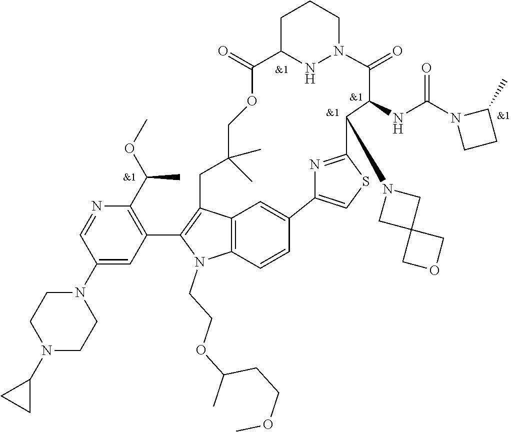 [SMILES: COCCC(C)OCCn1c(-c2cc(N3CCN(C4CC4)CC3)cnc2[C@H](C)OC)c2c3cc(ccc31)-c1csc(n1)[C@@H](N1CC3(COC3)C1)[C@H](NC(=O)N1CC[C@H]1C)C(=O)N1CCCC(N1)C(=O)OCC(C)(C)C2] |
| A292 | 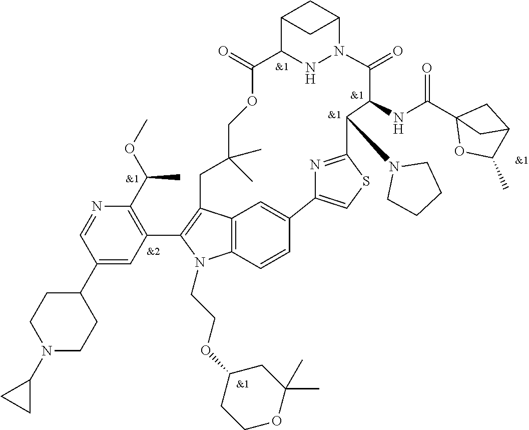 [SMILES: CO[C@@H](C)c1ncc(C2CCN(C3CC3)CC2)cc1-c1c2c3cc(ccc3n1CCO[C@H]1CCOC(C)(C)C1)-c1csc(n1)[C@@H](N1CCCC1)[C@H](NC(=O)C13CC(C1)[C@H](C)O3)C(=O)N1NC(C(=O)OCC(C)(C)C2)C2CC1C2] |
| A293 | 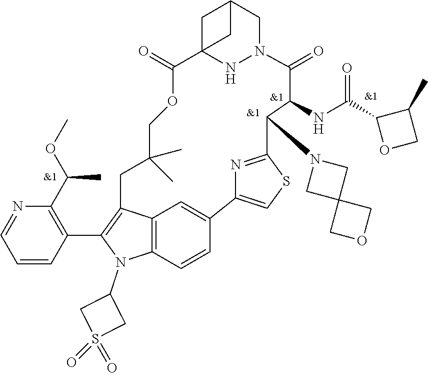 [SMILES: CO[C@@H](C)c1ncccc1-c1c2c3cc(ccc3n1C1CS(=O)(=O)C1)-c1csc(n1)[C@@H](N1CC3(COC3)C1)[C@H](NC(=O)[C@H]1OC[C@@H]1C)C(=O)N1CC3CC(C3)(N1)C(=O)OCC(C)(C)C2] |
| A294 | 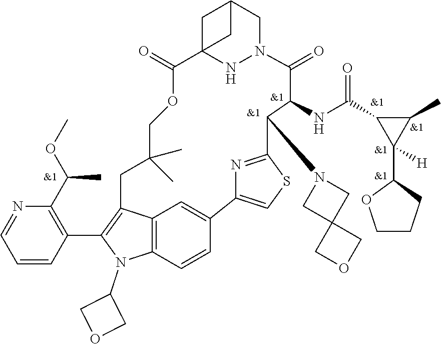 [SMILES: CO[C@@H](C)c1ncccc1-c1c2c3cc(ccc3n1C1COC1)-c1csc(n1)[C@@H](N1CC3(COC3)C1)[C@H](NC(=O)[C@@H]1[C@@H](C)[C@H]1[C@H]1CCCO1)C(=O)N1CC3CC(C3)(N1)C(=O)OCC(C)(C)C2] |
| A295 | 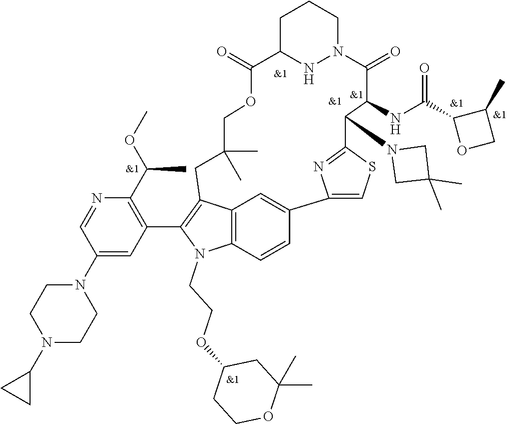 [SMILES: CO[C@@H](C)c1ncc(N2CCN(C3CC3)CC2)cc1-c1c2c3cc(ccc3n1CCO[C@H]1CCOC(C)(C)C1)-c1csc(n1)[C@@H](N1CC(C)(C)C1)[C@H](NC(=O)[C@H]1OC[C@@H]1C)C(=O)N1CCCC(N1)C(=O)OCC(C)(C)C2] |
| A296 | 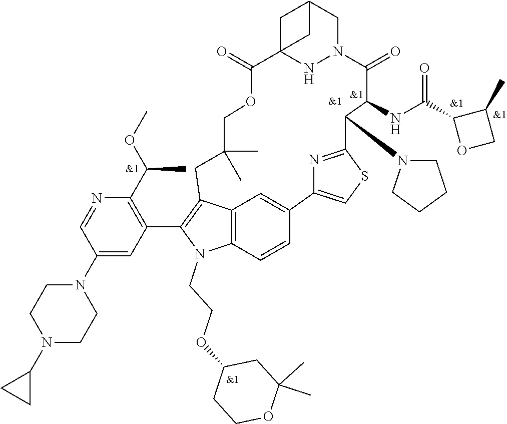 [SMILES: CO[C@@H](C)c1ncc(N2CCN(C3CC3)CC2)cc1-c1c2c3cc(ccc3n1CCO[C@H]1CCOC(C)(C)C1)-c1csc(n1)[C@@H](N1CCCC1)[C@H](NC(=O)[C@H]1OC[C@@H]1C)C(=O)N1CC3CC(C3)(N1)C(=O)OCC(C)(C)C2] |
| A297 | 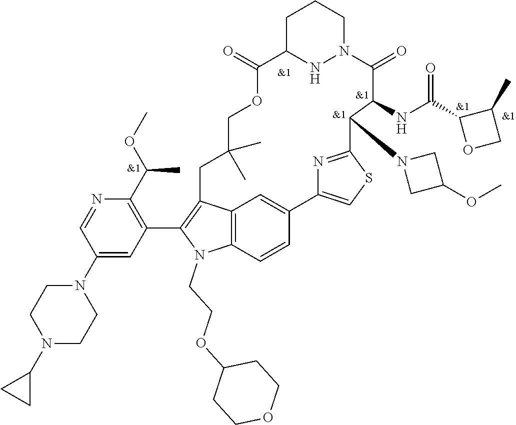 [SMILES: COC1CN([C@@H]2c3nc(cs3)-c3ccc4c(c3)c(c(-c3cc(N5CCN(C6CC6)CC5)cnc3[C@H](C)OC)n4CCOC3CCOCC3)CC(C)(C)COC(=O)C3CCCN(N3)C(=O)[C@H]2NC(=O)[C@H]2OC[C@@H]2C)C1] |
| A298 | 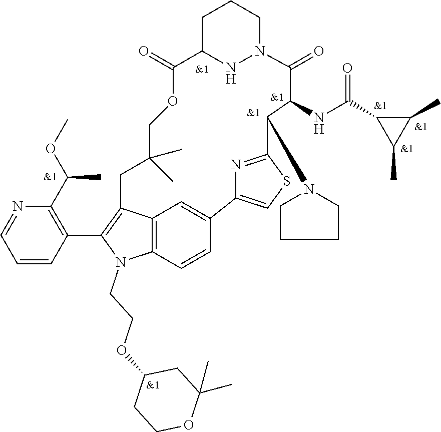 [SMILES: CO[C@@H](C)c1ncccc1-c1c2c3cc(ccc3n1CCO[C@H]1CCOC(C)(C)C1)-c1csc(n1)[C@@H](N1CCCC1)[C@H](NC(=O)[C@H]1[C@H](C)[C@@H]1C)C(=O)N1CCCC(N1)C(=O)OCC(C)(C)C2] |
| A299 | 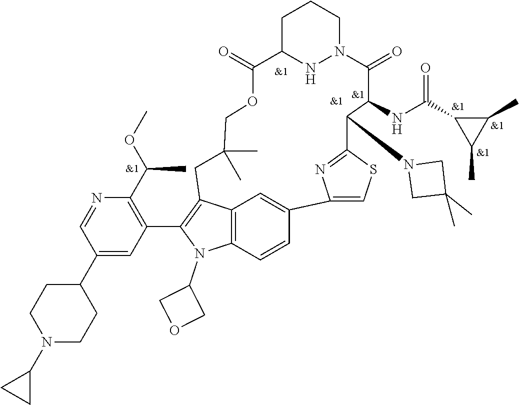 [SMILES: CO[C@@H](C)c1ncc(C2CCN(C3CC3)CC2)cc1-c1c2c3cc(ccc3n1C1COC1)-c1csc(n1)[C@@H](N1CC(C)(C)C1)[C@H](NC(=O)[C@H]1[C@H](C)[C@@H]1C)C(=O)N1CCCC(N1)C(=O)OCC(C)(C)C2] |
| A300 | 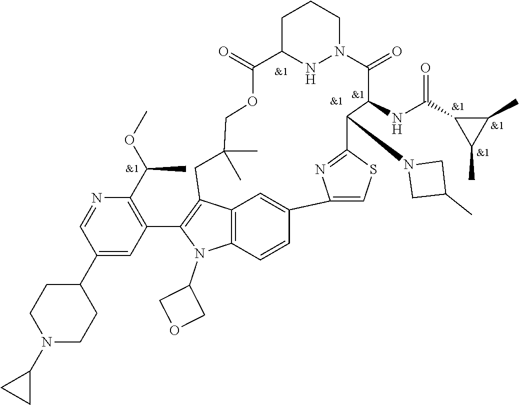 [SMILES: CO[C@@H](C)c1ncc(C2CCN(C3CC3)CC2)cc1-c1c2c3cc(ccc3n1C1COC1)-c1csc(n1)[C@@H](N1CC(C)C1)[C@H](NC(=O)[C@H]1[C@H](C)[C@@H]1C)C(=O)N1CCCC(N1)C(=O)OCC(C)(C)C2] |
| A301 | 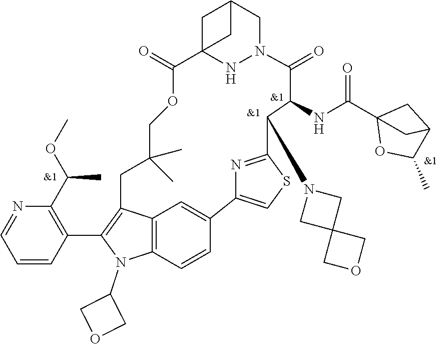 [SMILES: CO[C@@H](C)c1ncccc1-c1c2c3cc(ccc3n1C1COC1)-c1csc(n1)[C@@H](N1CC3(COC3)C1)[C@H](NC(=O)C13CC(C1)[C@H](C)O3)C(=O)N1CC3CC(C3)(N1)C(=O)OCC(C)(C)C2] |
| A302 | 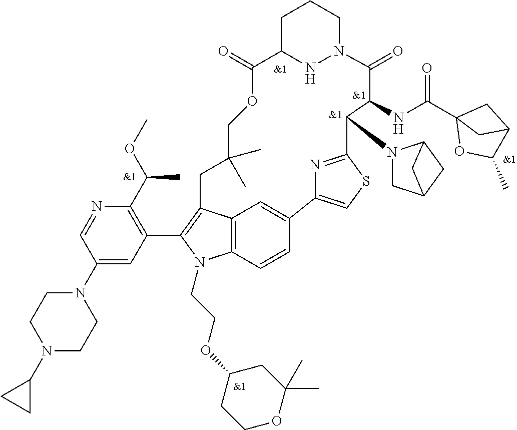 [SMILES: CO[C@@H](C)c1ncc(N2CCN(C3CC3)CC2)cc1-c1c2c3cc(ccc3n1CCO[C@H]1CCOC(C)(C)C1)-c1csc(n1)[C@@H](N1CC3CC1C3)[C@H](NC(=O)C13CC(C1)[C@H](C)O3)C(=O)N1CCCC(N1)C(=O)OCC(C)(C)C2] |
| A303 | 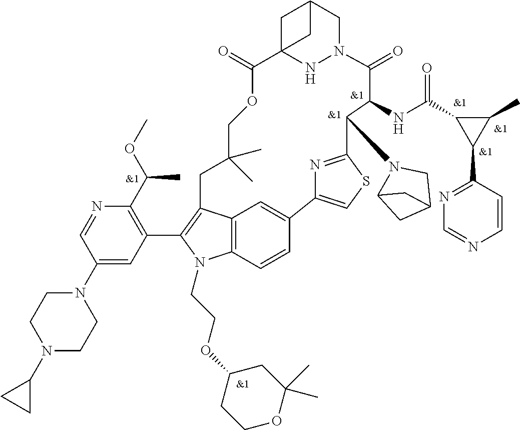 [SMILES: CO[C@@H](C)c1ncc(N2CCN(C3CC3)CC2)cc1-c1c2c3cc(ccc3n1CCO[C@H]1CCOC(C)(C)C1)-c1csc(n1)[C@@H](N1CC3CC1C3)[C@H](NC(=O)[C@@H]1[C@@H](C)[C@H]1c1ccncn1)C(=O)N1CC3CC(C3)(N1)C(=O)OCC(C)(C)C2] |
| A304 | 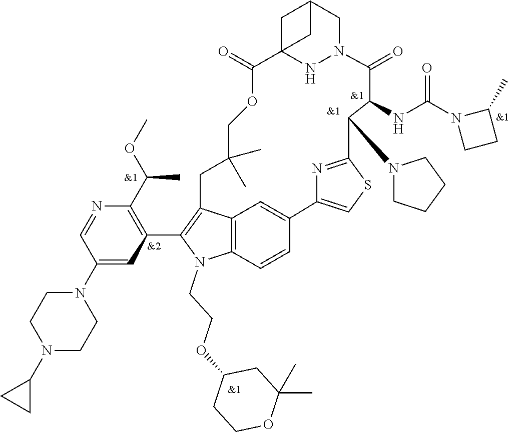 |
| A305 | 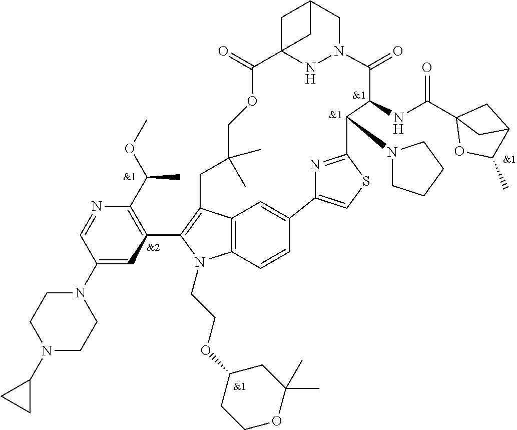 |
| A306 | 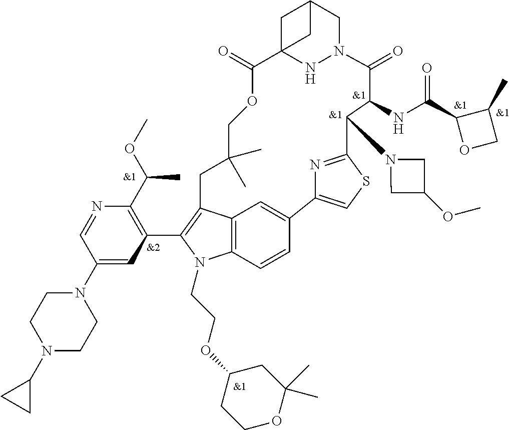 |
| A307 | 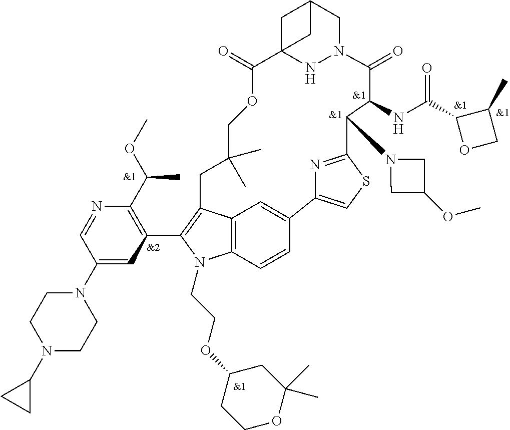 |
| A308 | 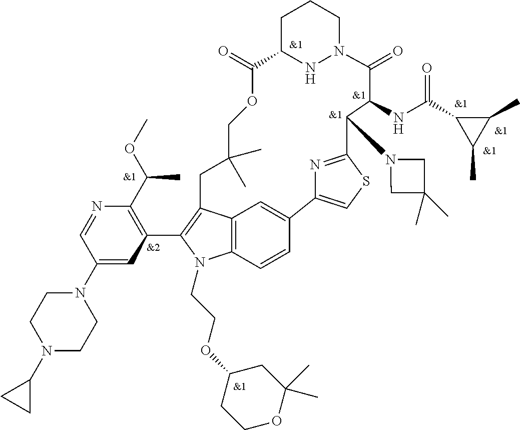 |
| A309 | 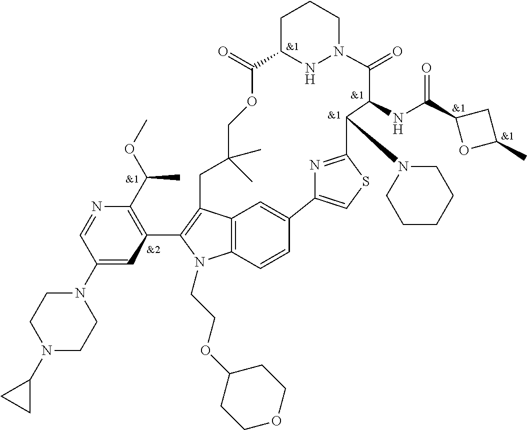 |
| A310 | 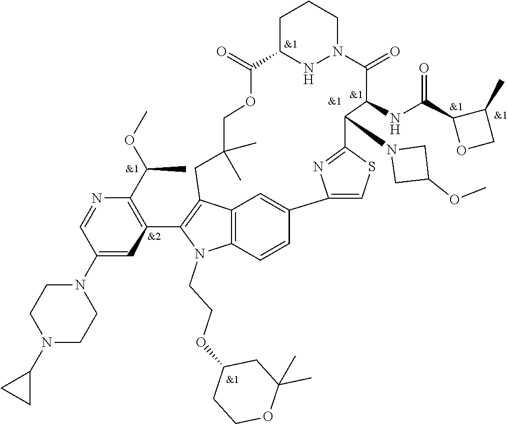 |
| A311 | 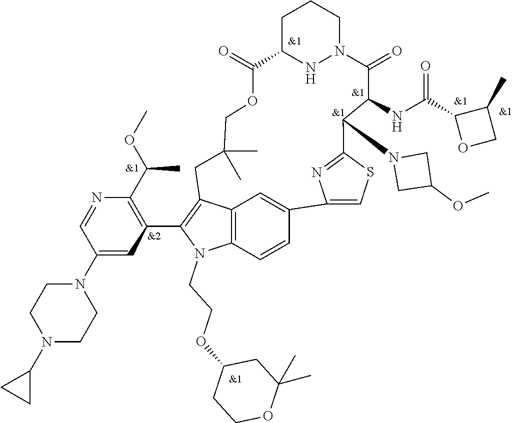 |
| A312 | 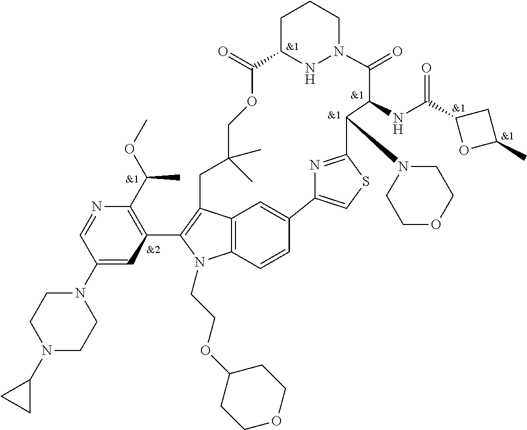 |
| A313 | 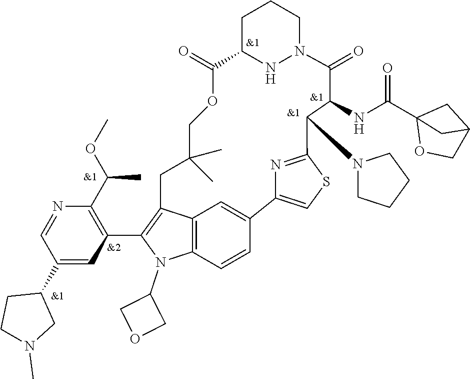 |
| A314 | 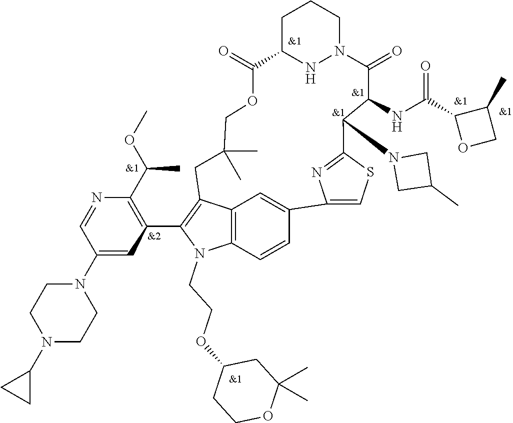 |
| A315 | 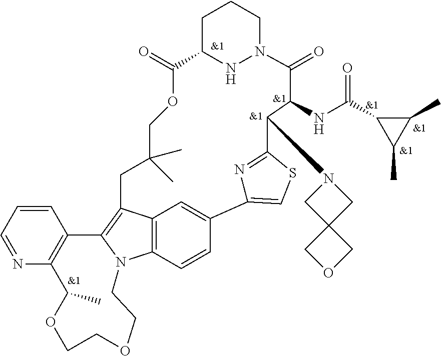 [SMILES: C[C@@H]1[C@H](C)[C@H]1C(=O)N[C@@H]1C(=O)N2CCC[C@H](N2)C(=O)OCC(C)(C)Cc2c3n(c4ccc(cc24)-c2csc(n2)[C@H]1N1CC2(COC2)C1)CCOCCO[C@@H](C)c1ncccc1-3] |
| A316 | 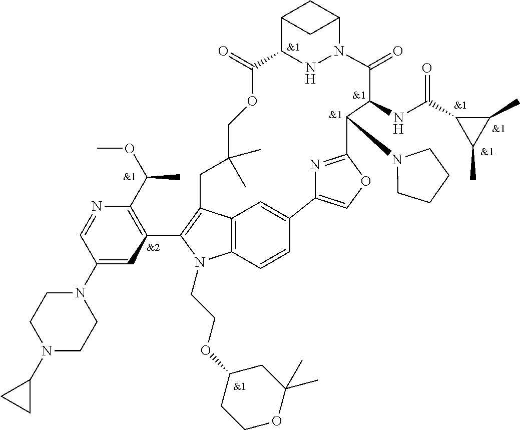 |
| A317 | 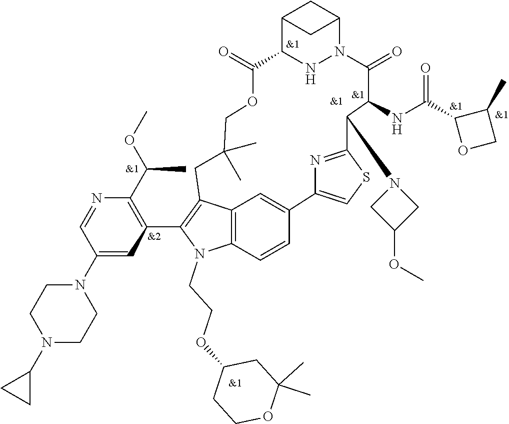 |
| A318 | 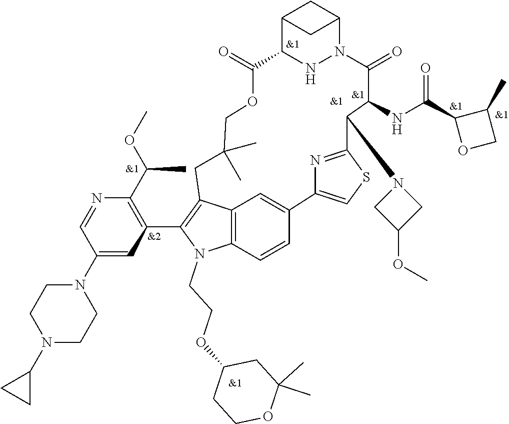 |
| A319 | 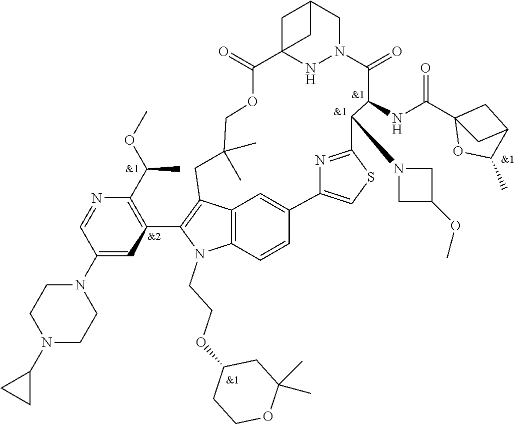 |
| A320 | 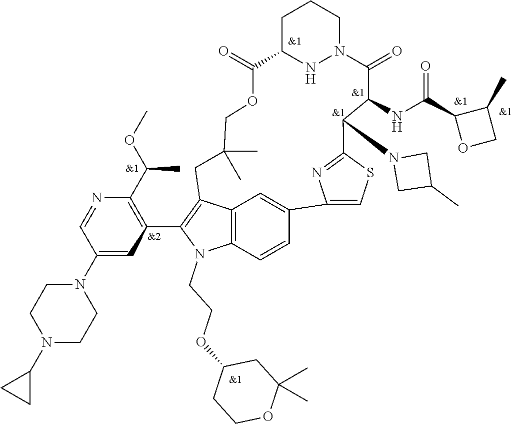 |
| A321 | 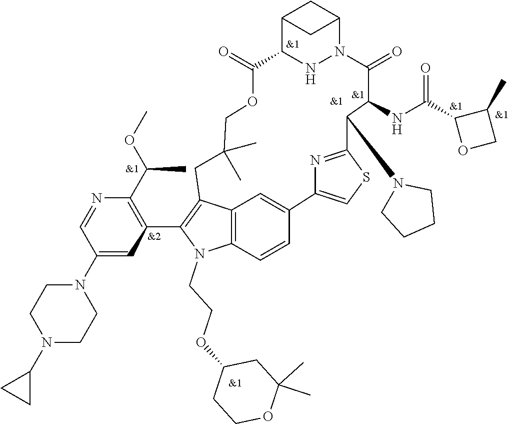 |
| A322 | 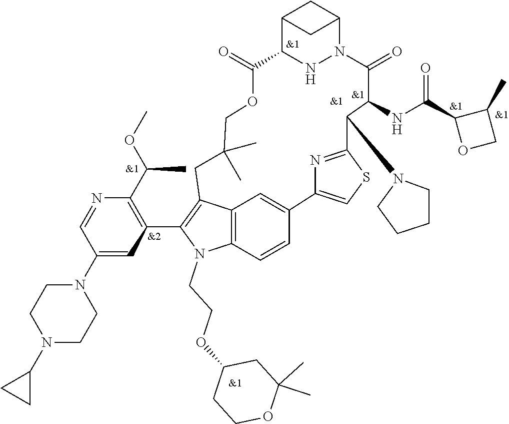 |
| A323 | 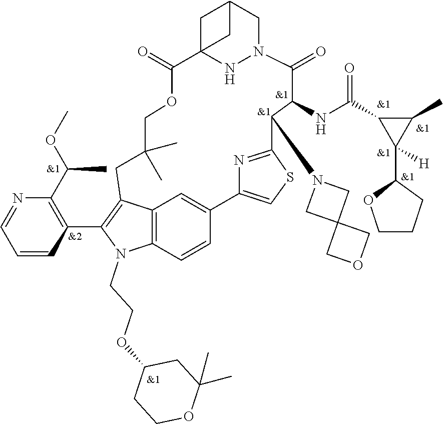 |
| A324 | 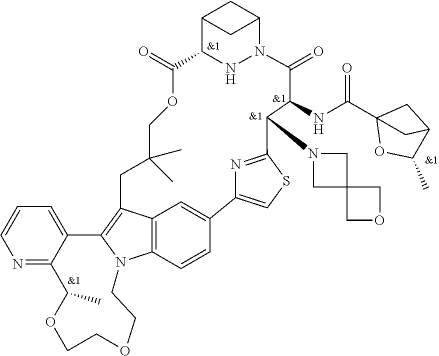 [SMILES: C[C@@H]1OCCOCCn2c(c3c4cc(ccc42)-c2csc(n2)[C@@H](N2CC4(COC4)C2)[C@H](NC(=O)C24CC(C2)[C@H](C)O4)C(=O)N2N[C@H](C(=O)OCC(C)(C)C3)C3CC2C3)-c2cccnc21] |
| A325 | 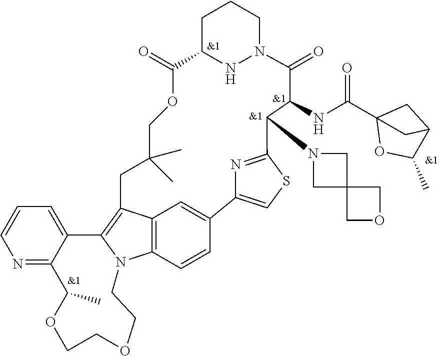 [SMILES: C[C@@H]1OCCOCCn2c(c3c4cc(ccc42)-c2csc(n2)[C@@H](N2CC4(COC4)C2)[C@H](NC(=O)C24CC(C2)[C@H](C)O4)C(=O)N2CCC[C@H](N2)C(=O)OCC(C)(C)C3)-c2cccnc21] |
| A326 | 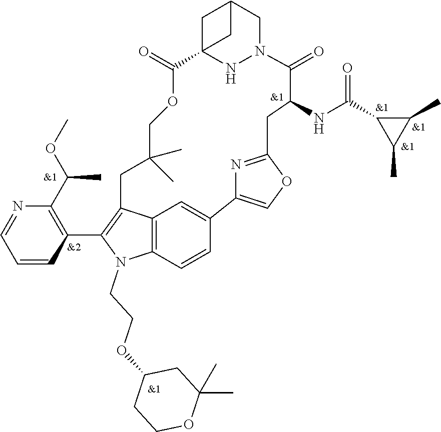 |
| A327 | 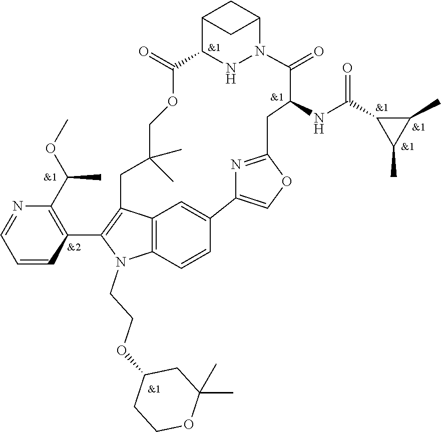 |
| A328 | 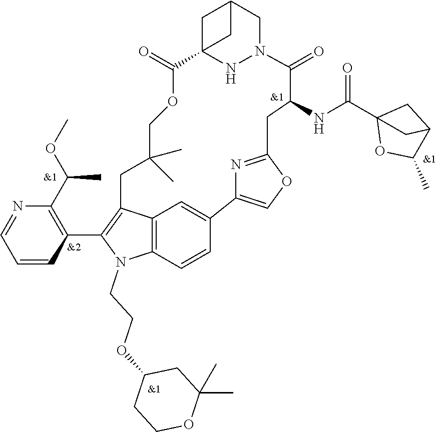 |
| A329 | 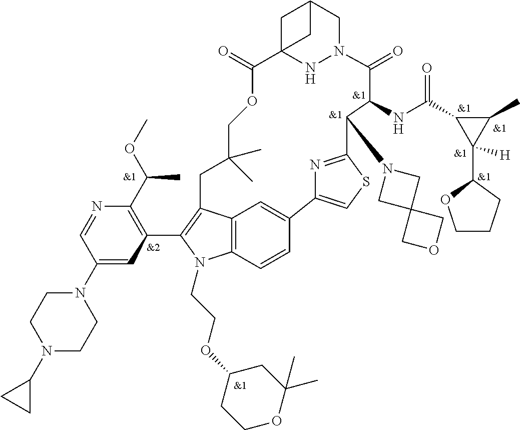 |
| A330 | 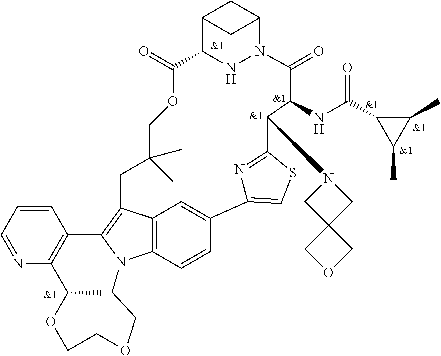 [SMILES: C[C@@H]1[C@H](C)[C@H]1C(=O)N[C@@H]1C(=O)N2N[C@H](C(=O)OCC(C)(C)Cc3c4n(c5ccc(cc35)-c3csc(n3)[C@H]1N1CC3(COC3)C1)CCOCCO[C@@H](C)c1ncccc1-4)C1CC2C1] |
| A331 | 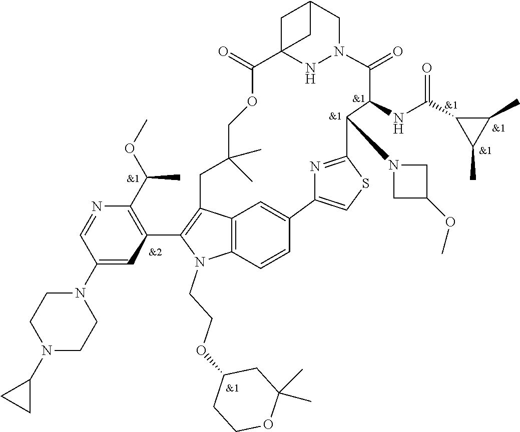 |
| A332 | 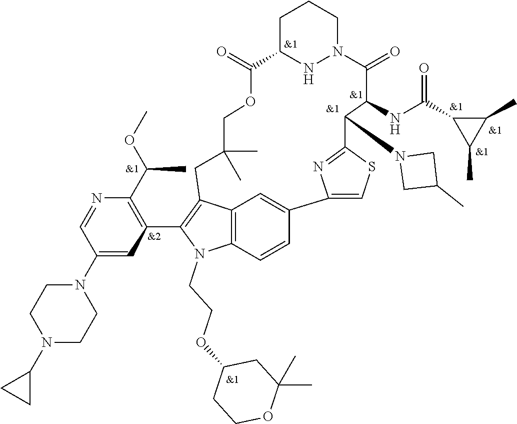 |
| A333 | 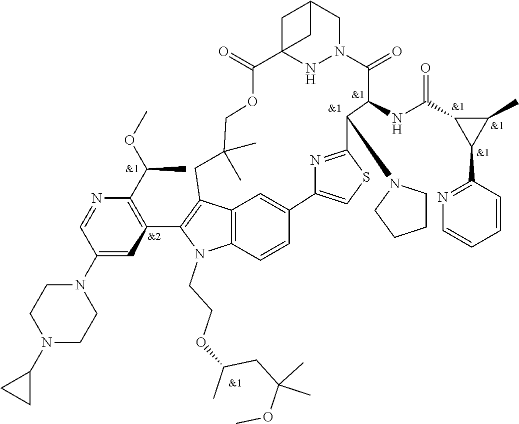 |
| A334 | 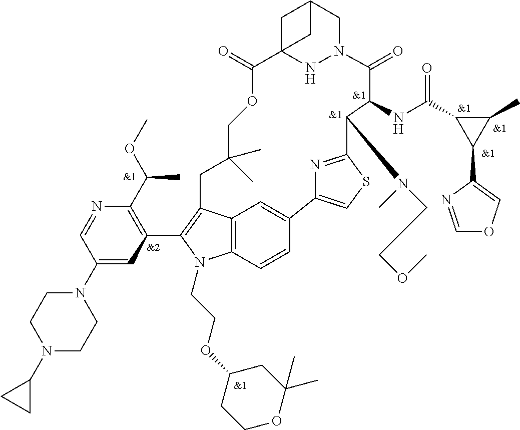 |
| A335 | 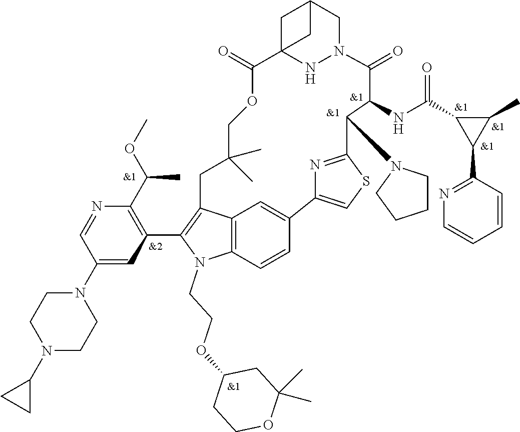 |
| A336 | 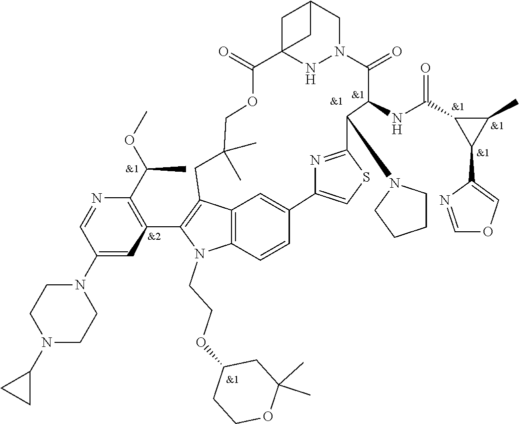 |
| A337 | 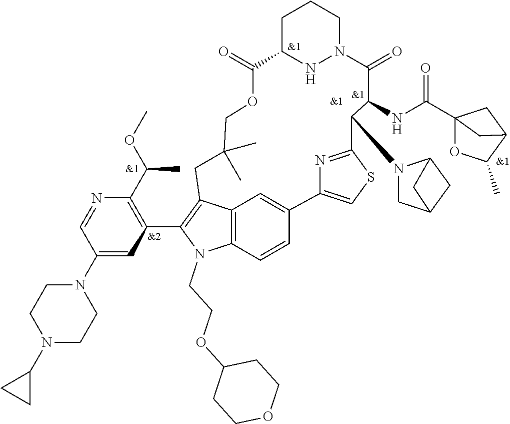 |
| A338 | 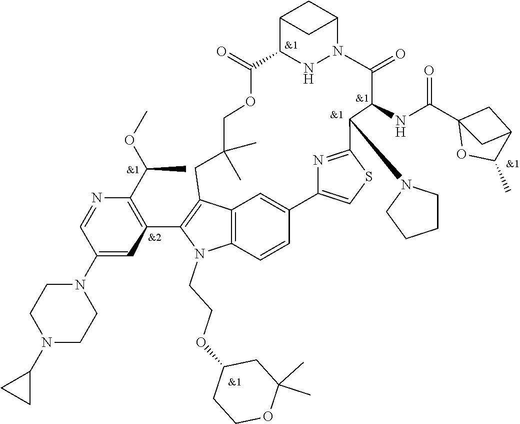 |
| A339 | 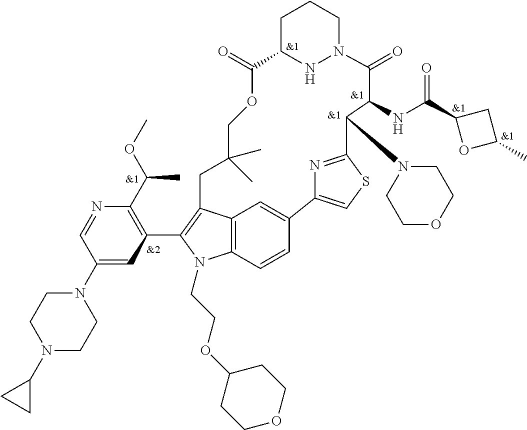 |
| A340 | 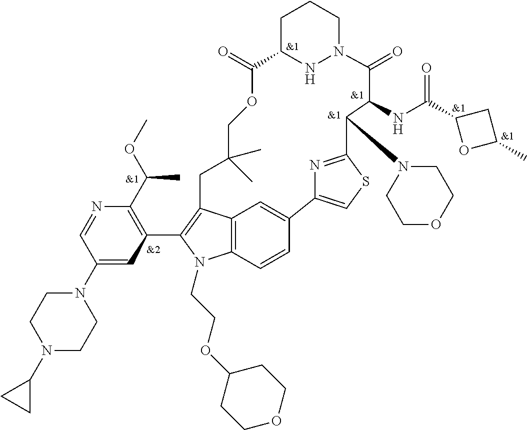 |
| A341 | 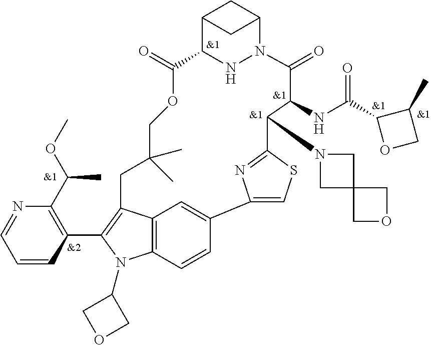 |
| A342 | 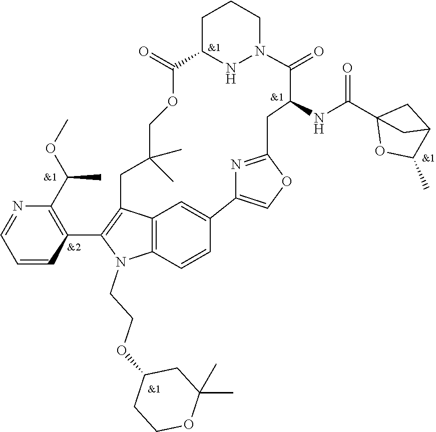 |
| A343 | 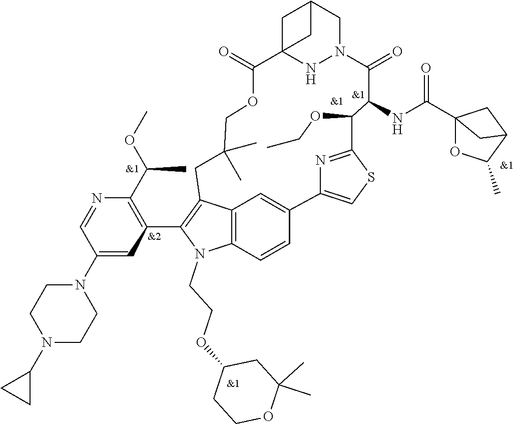 |
| A344 | 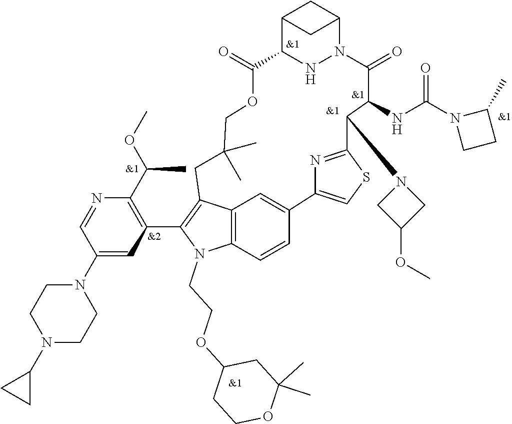 |
| A345 | 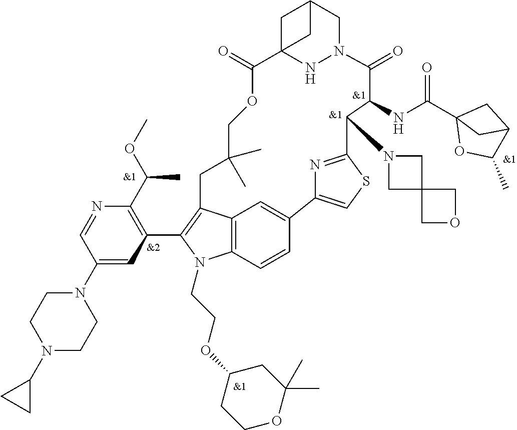 |
| A346 | 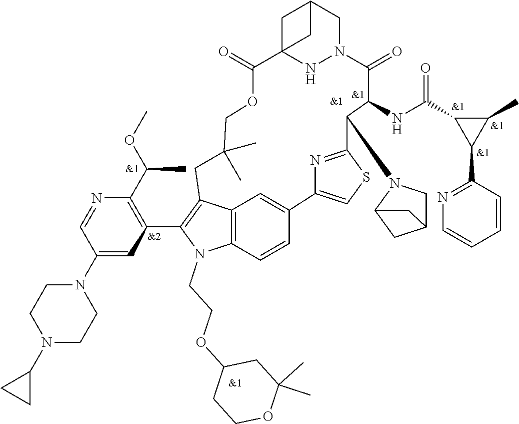 |
| A347 | 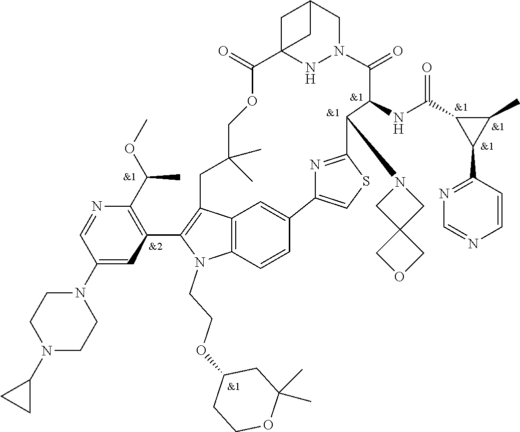 |
| A348 | 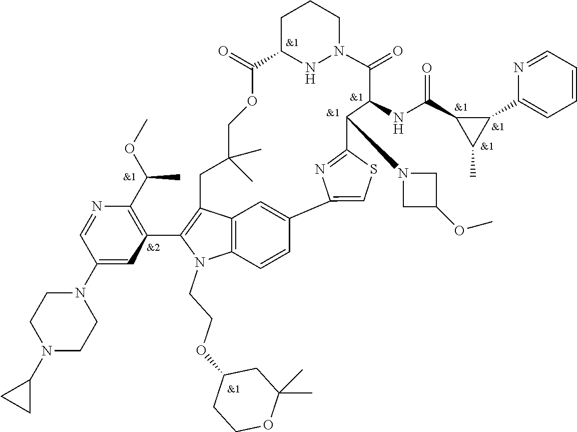 |
| A349 | 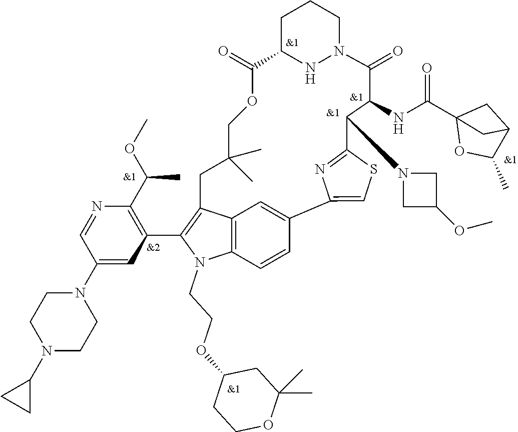 |
| A350 | 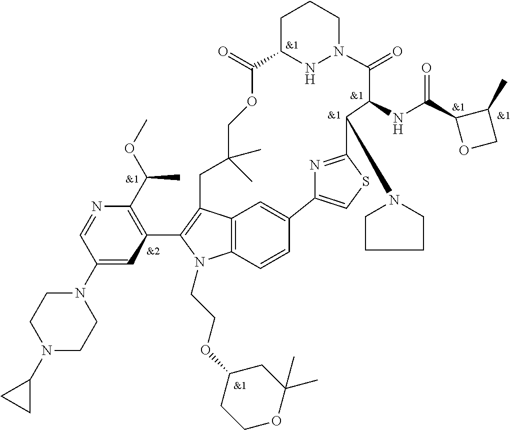 |
| A351 | 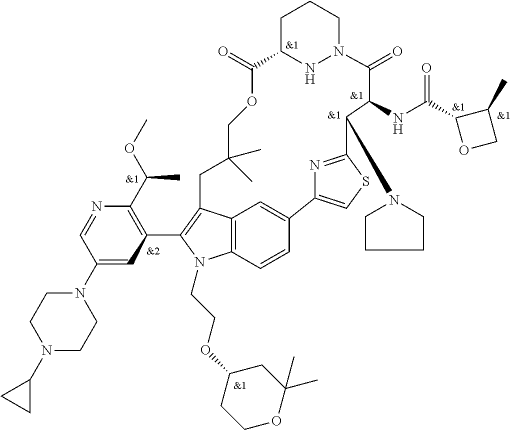 |
| A352 | 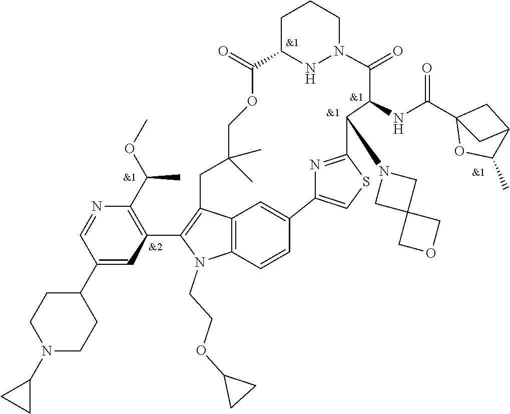 |
| A353 | 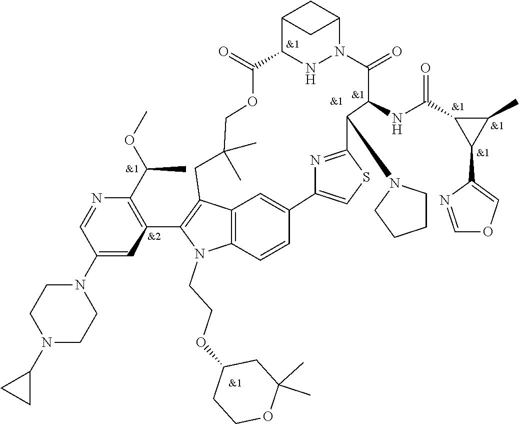 |
| A354 | 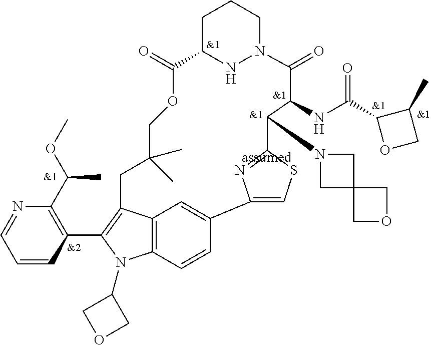 |
| A355 | 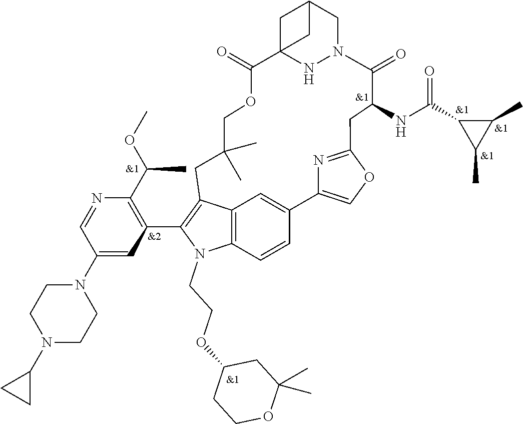 |
| A356 | 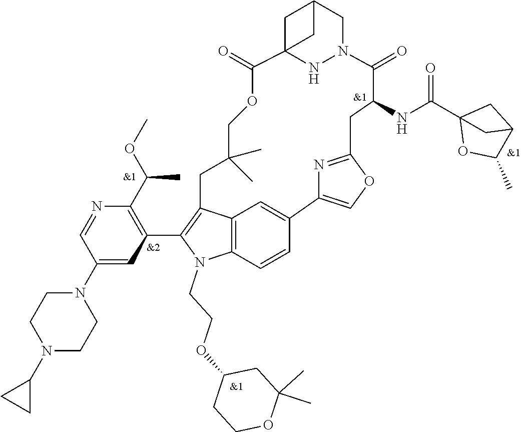 |
| A357 | 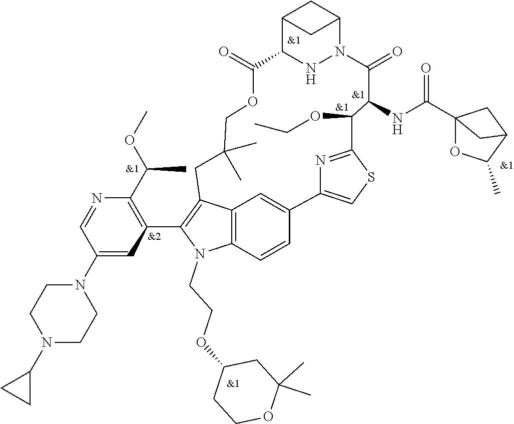 |
| A358 | 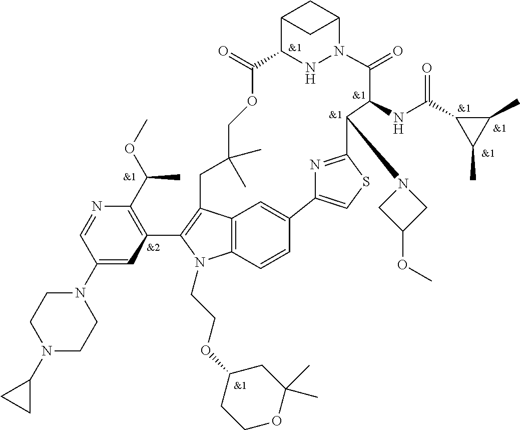 |
| A359 | 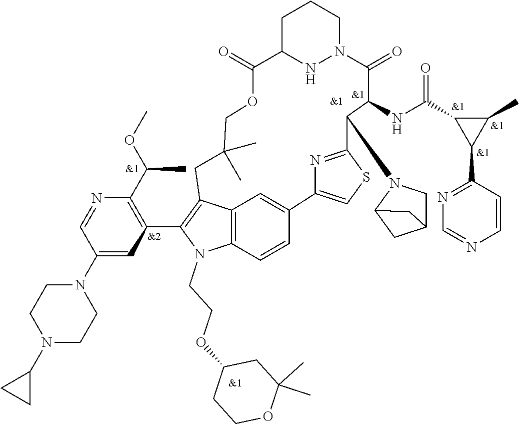 |
| A360 | 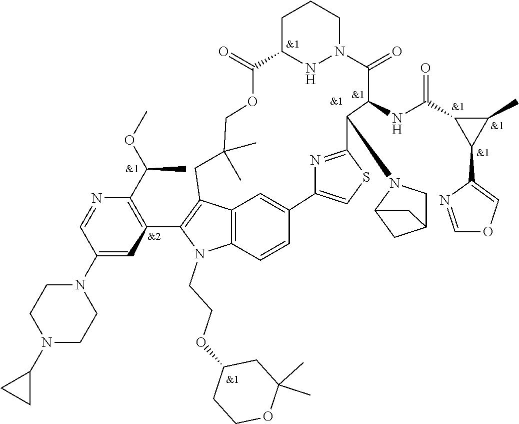 |
| A361 | 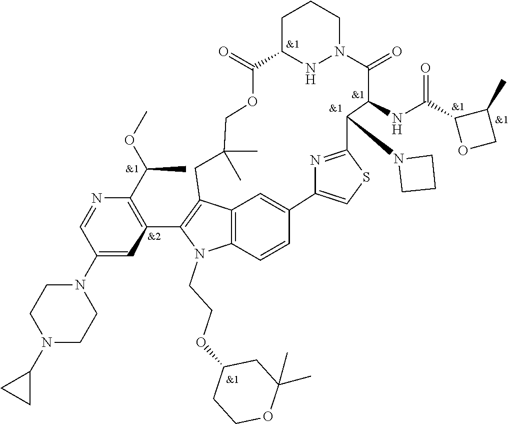 |
| A362 | 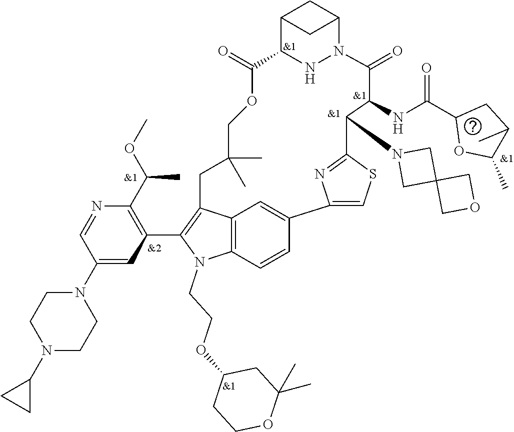 |
| A363 | 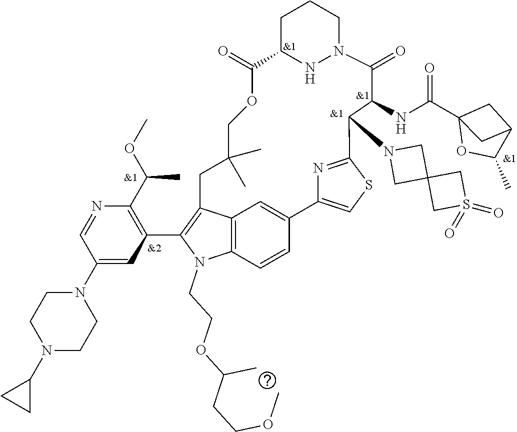 |
| A364 | 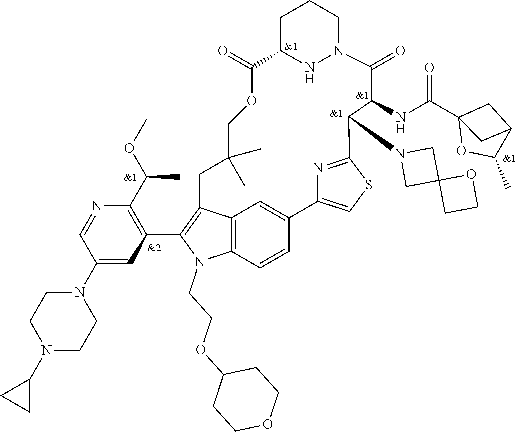 |
| A365 | 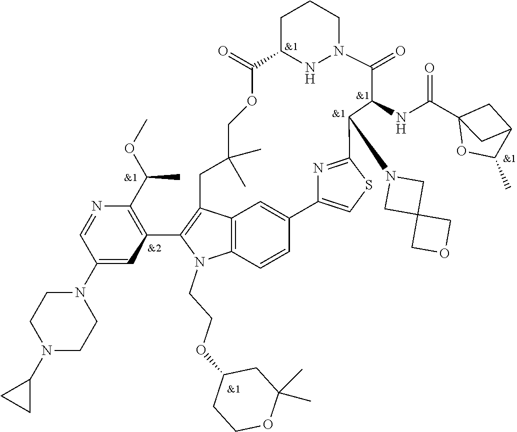 |
| A366 | 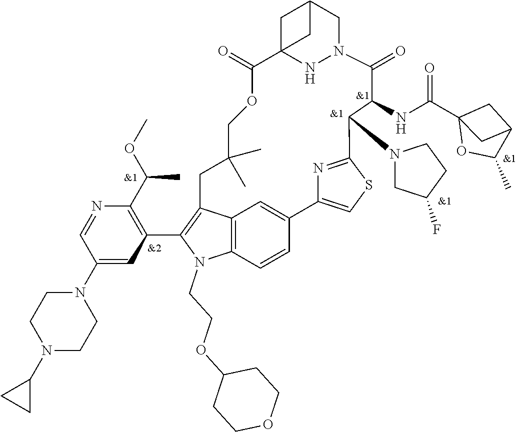 |
| A367 | 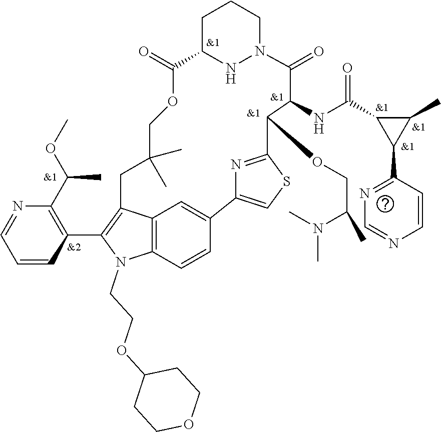 |
| A368 | 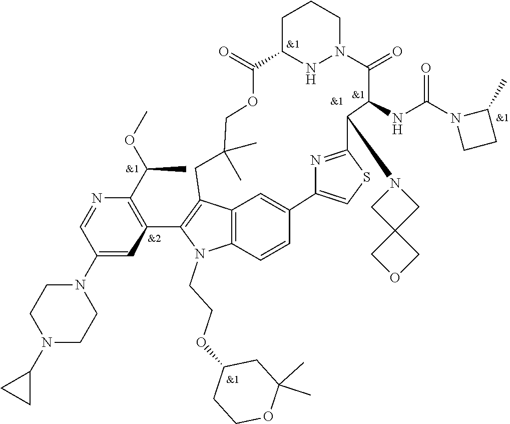 |
| A369 | 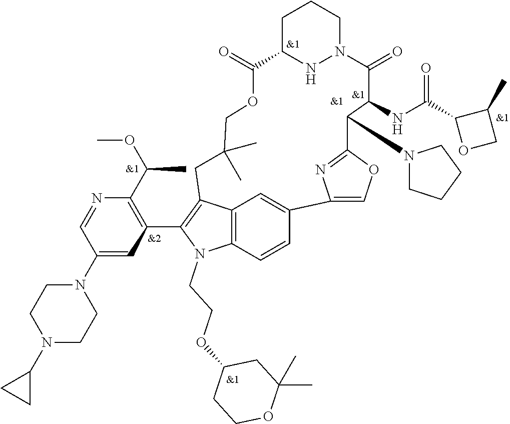 |
| A370 | 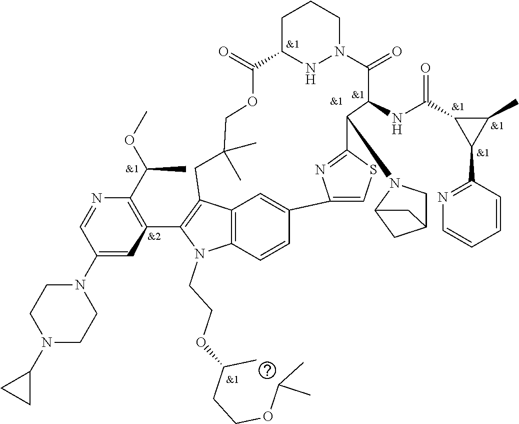 |
| A371 | 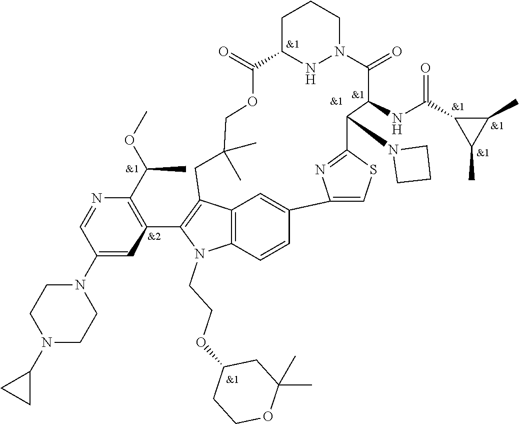 |
| A372 | 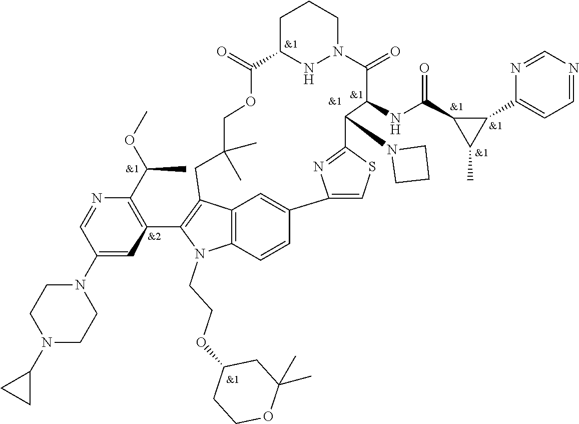 |
| A373 | 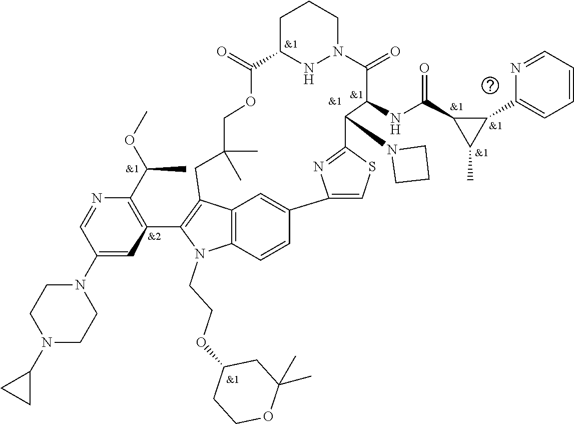 |
| A374 | 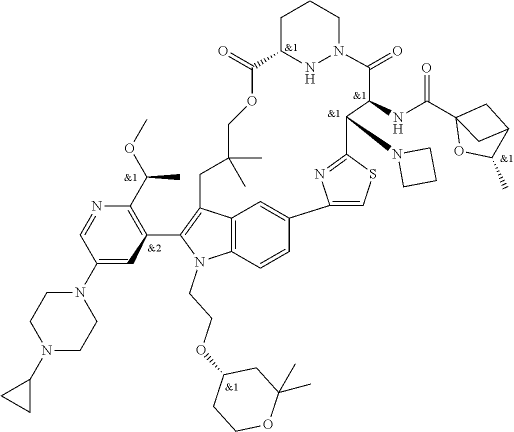 |
| A375 | 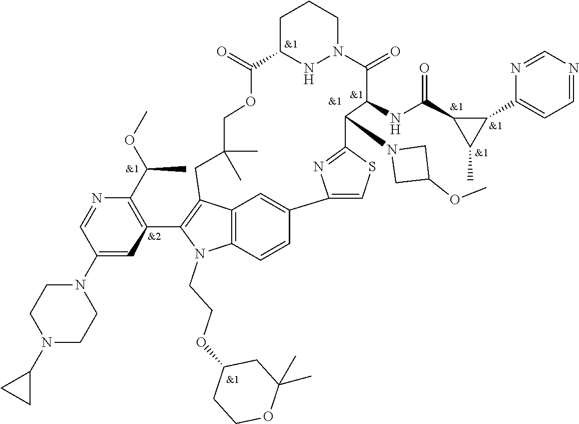 |
| A376 | 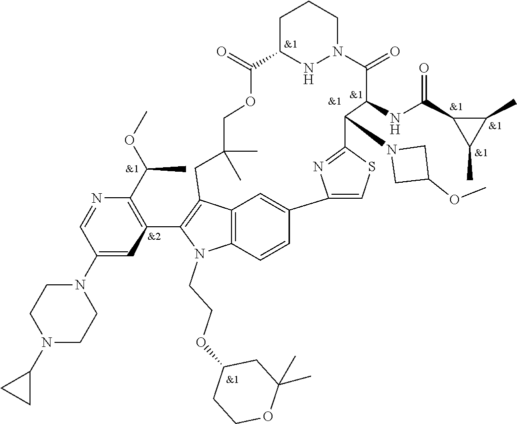 |
| A377 | 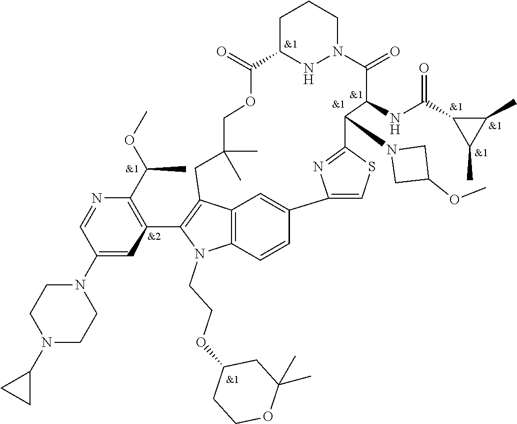 |
| A378 | 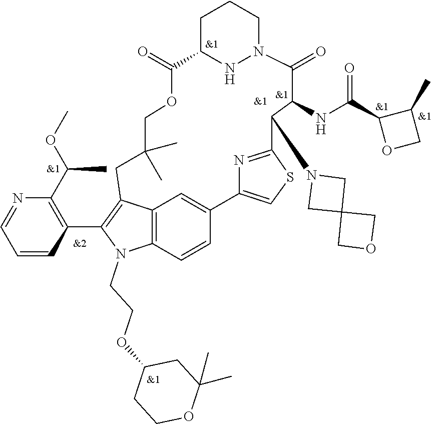 |
| A379 | 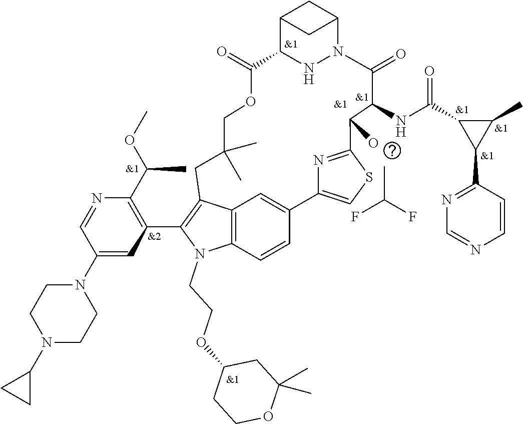 |
| A380 | 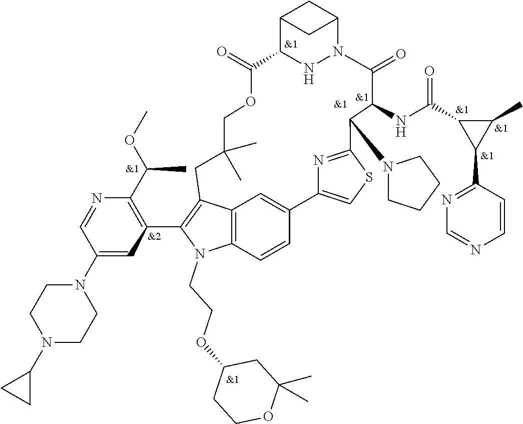 |
| A381 | 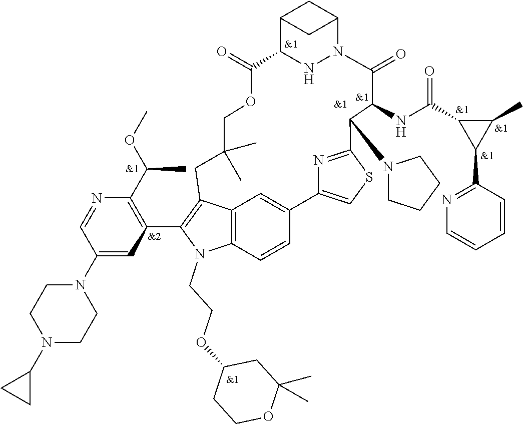 |
| A382 | 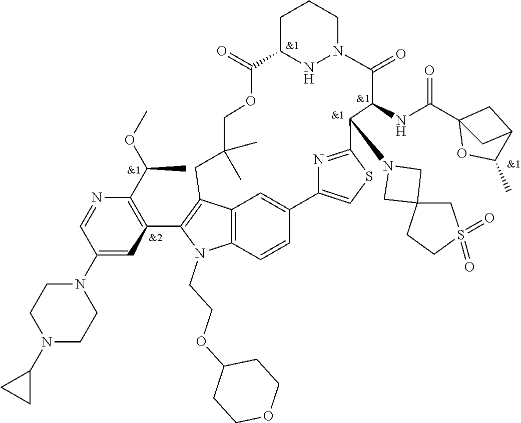 |
| A383 | 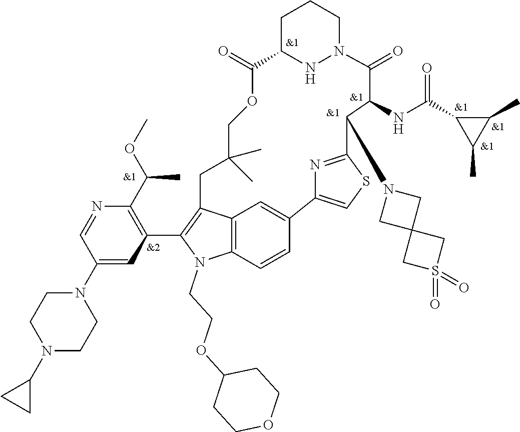 |
| A384 | 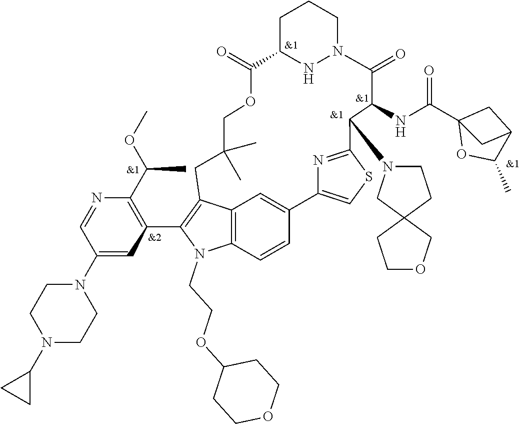 |
| A385 | 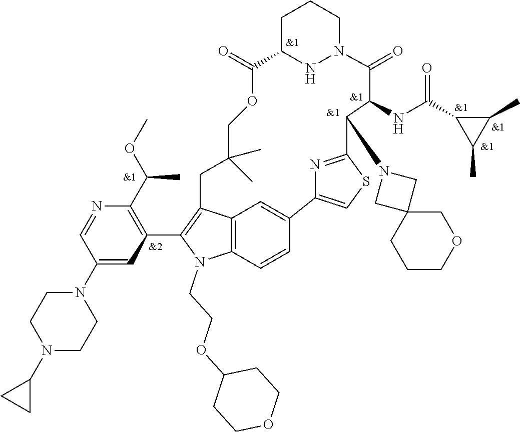 |
| A386 | 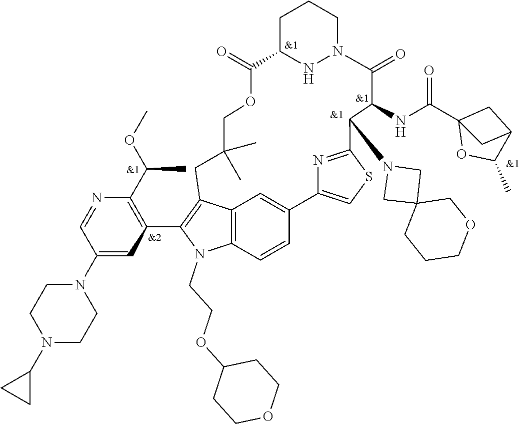 |
| A387 | 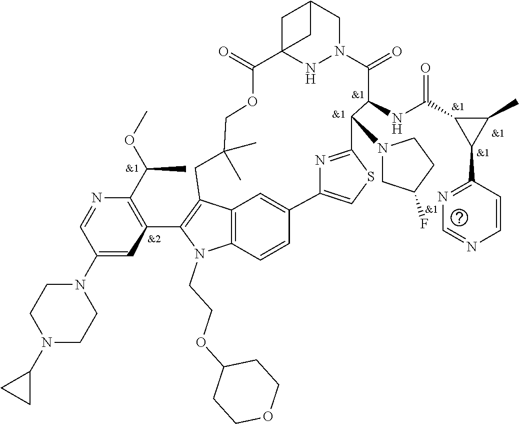 |
| A388 | 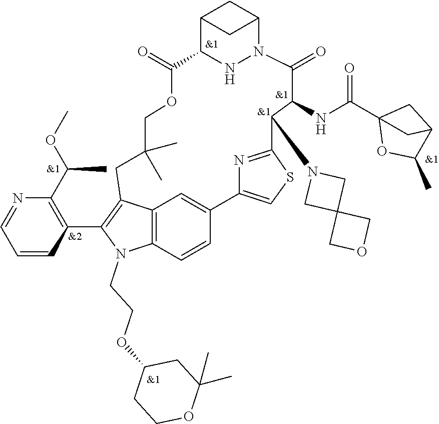 |
| A389 | 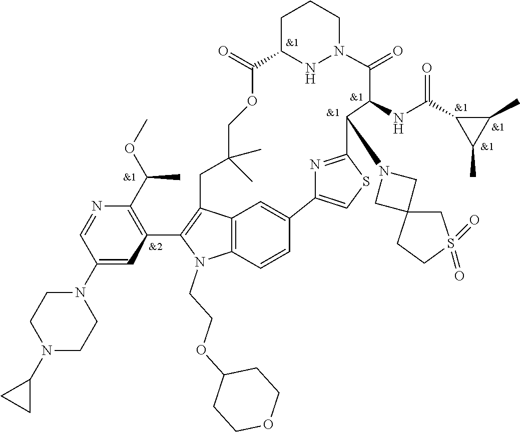 |
| A390 | 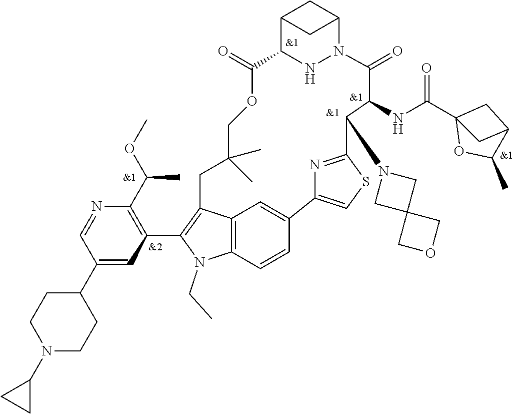 |
| A391 | 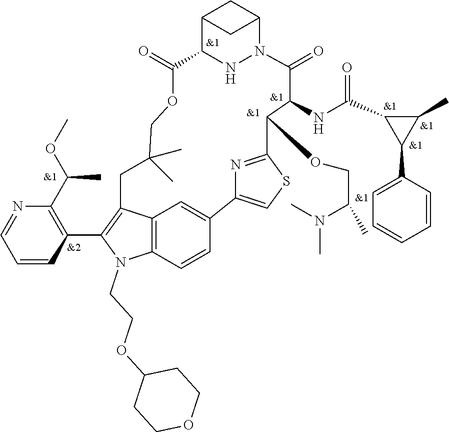 |
| A392 | 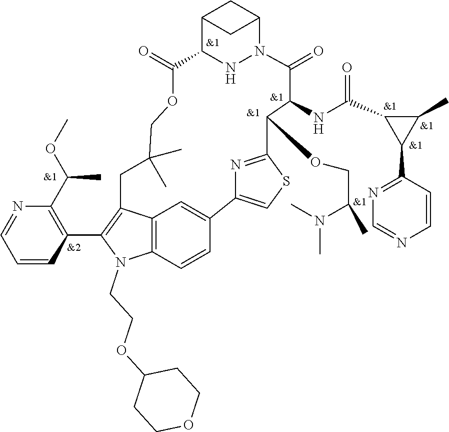 |
| A393 | 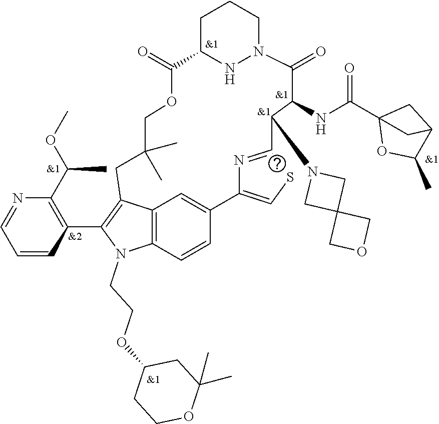 |
| A394 | 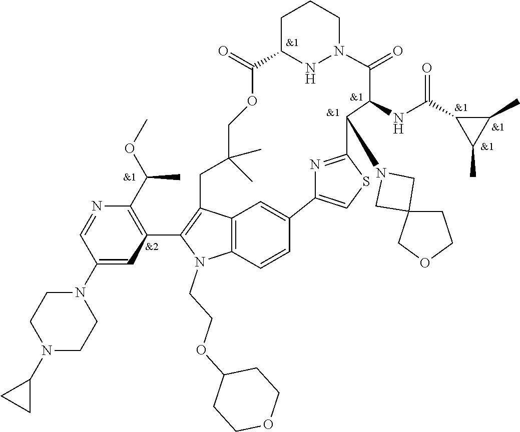 |
| A395 | 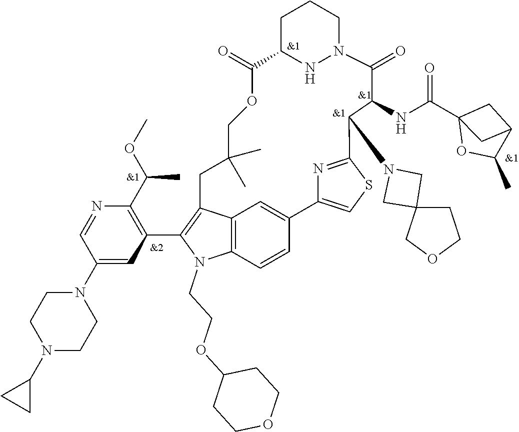 |
| A396 | 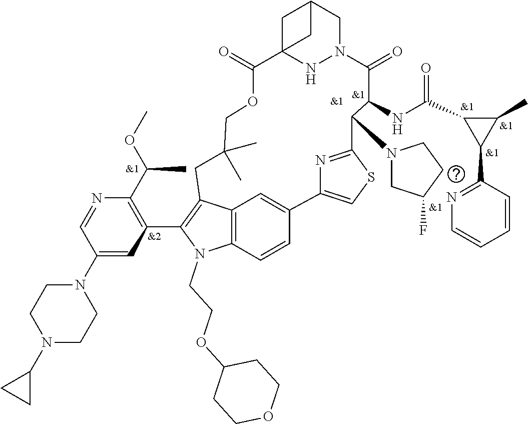 |
| A397 | 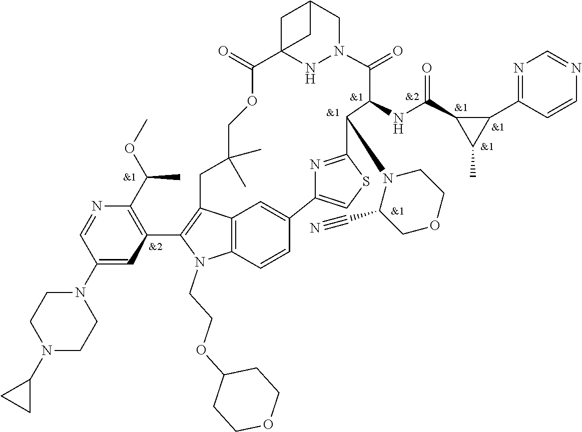 |
| A398 | 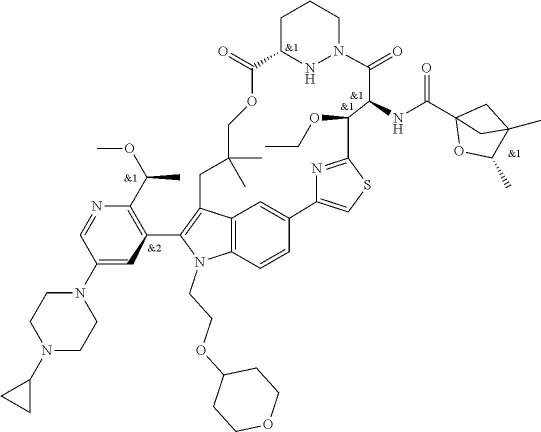 |
| A399 | 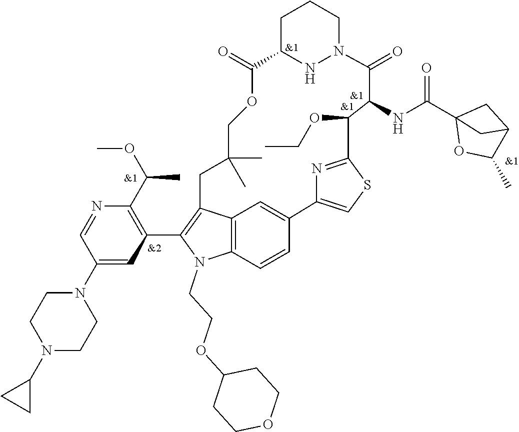 |
| A400 | 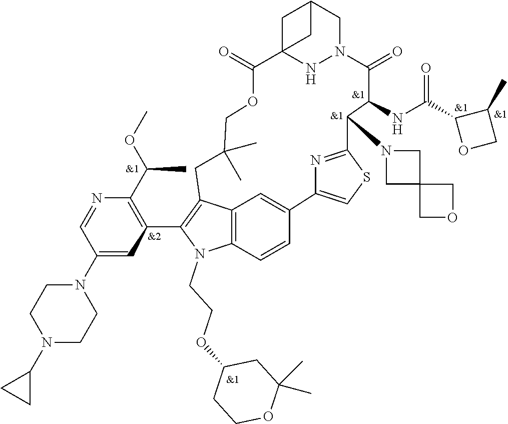 |
| A401 | 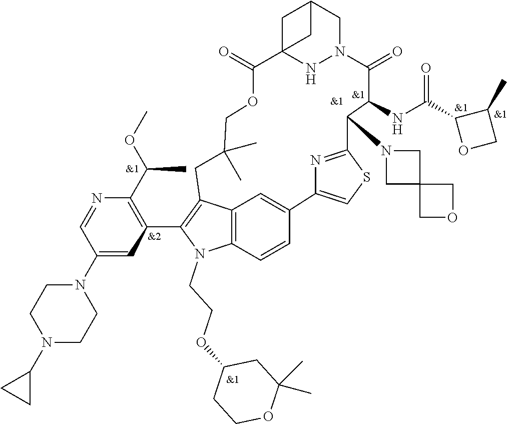 |
| A402 | 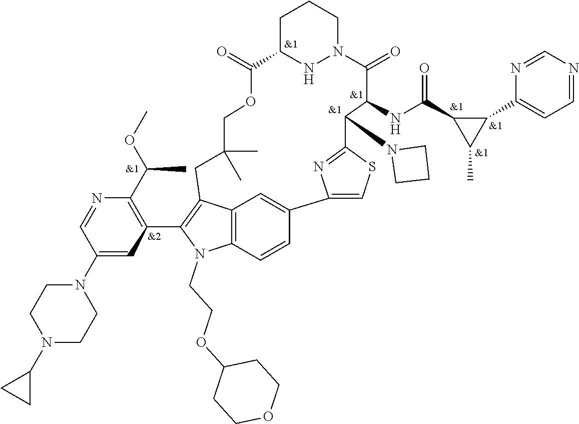 |
| A403 | 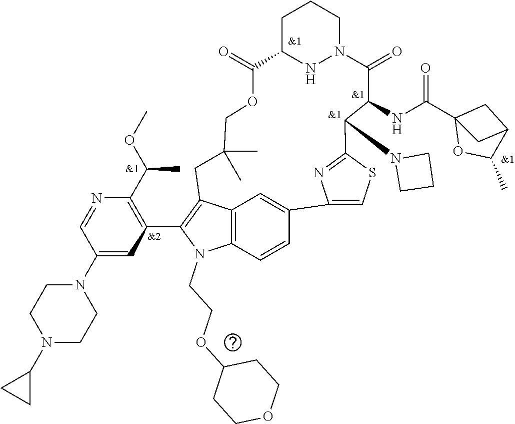 |
| A404 | 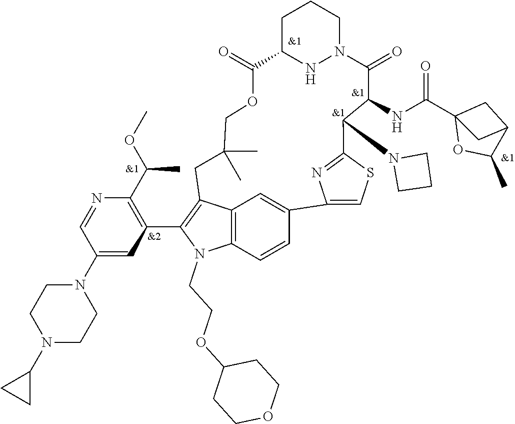 |
| A405 | 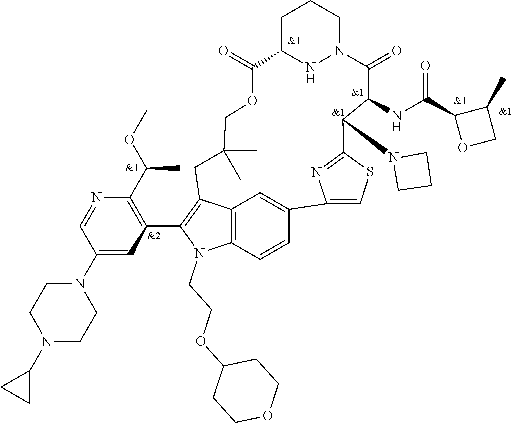 |
| A406 | 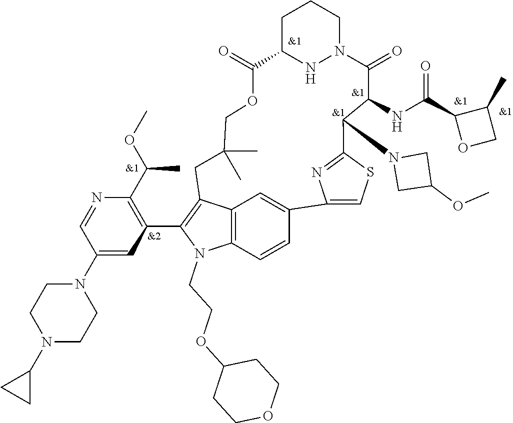 |
| A407 | 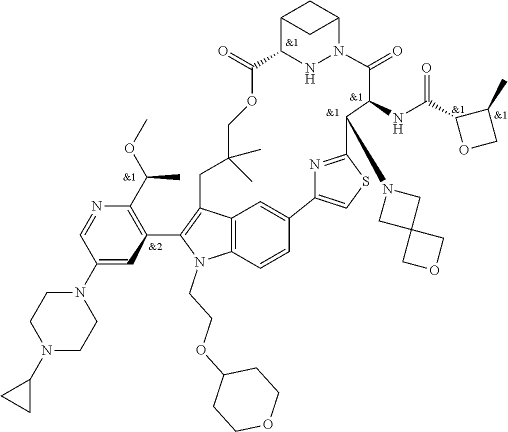 |
| A408 | 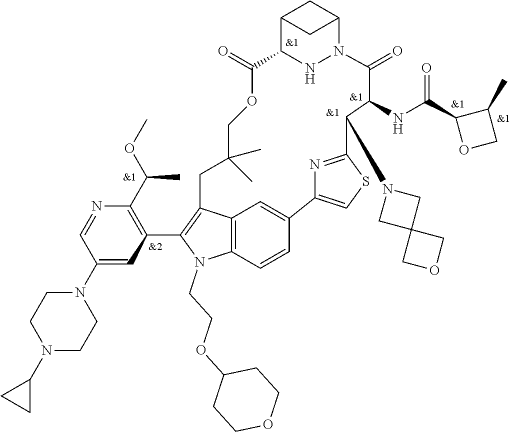 |
| A409 | 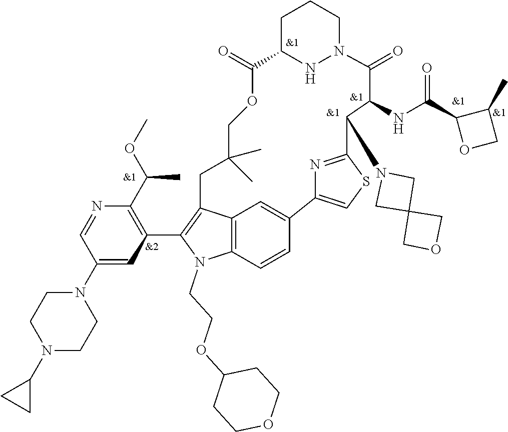 |
| A410 | 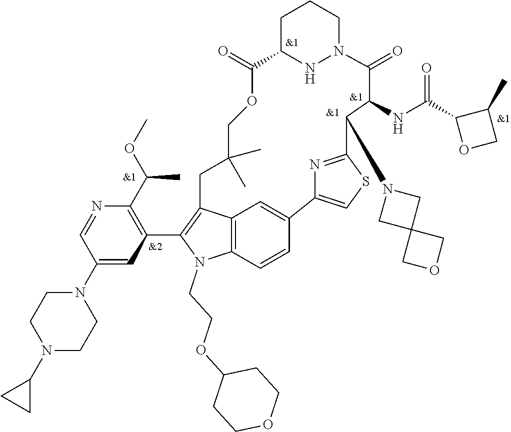 |
| A411 | 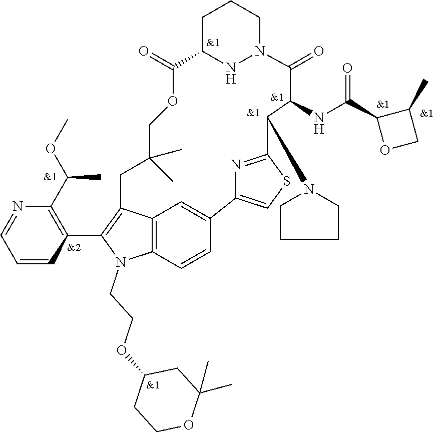 |
| A412 | 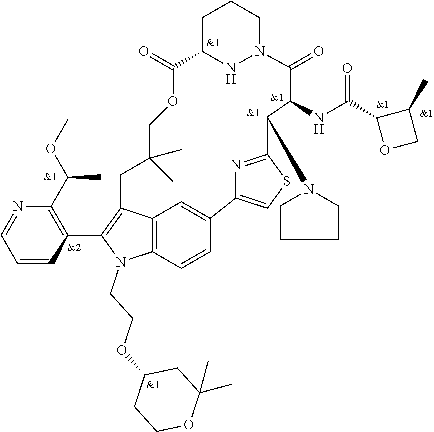 |
| A413 | 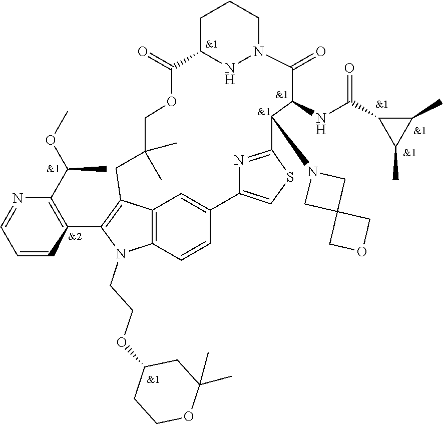 |
| A414 | 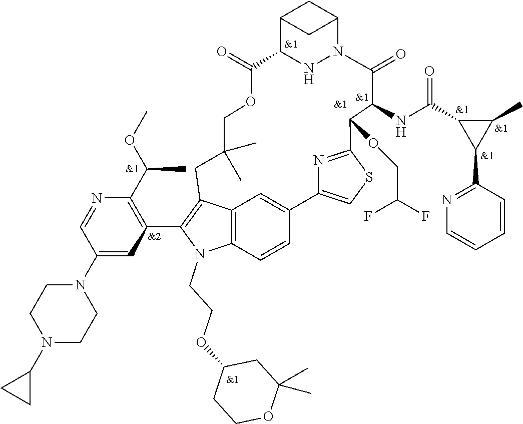 |
| A415 | 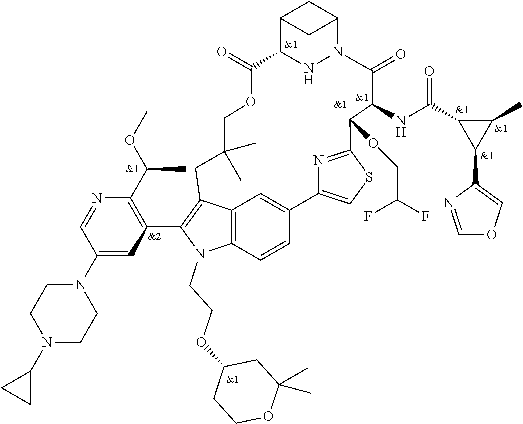 |
| A416 | 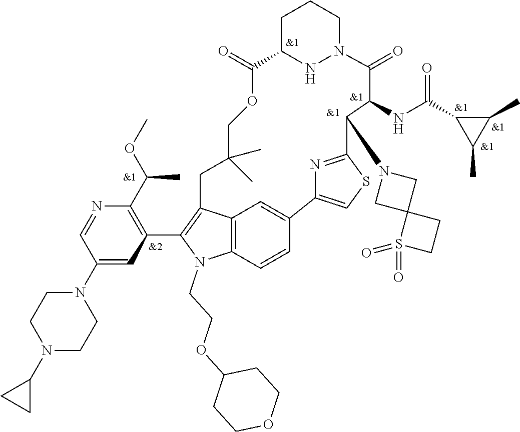 |
| A417 | 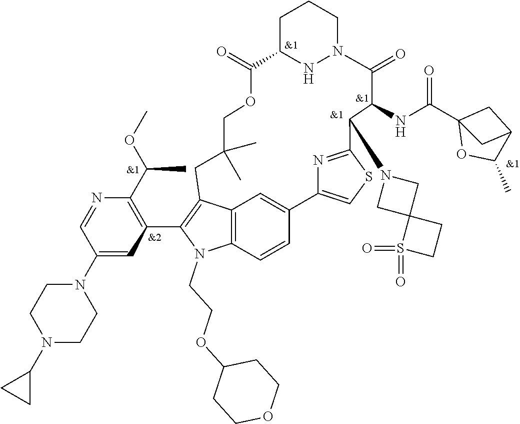 |
| A418 | 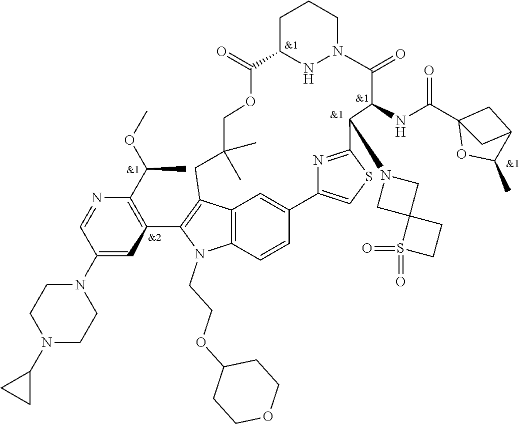 |
| A419 | 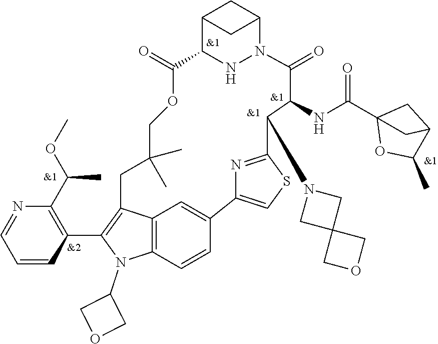 |
| A420 | 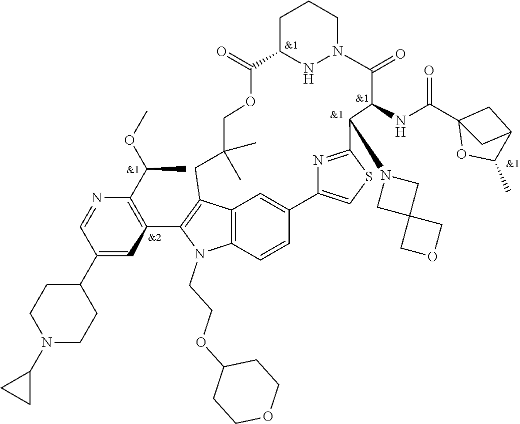 |
| A421 | 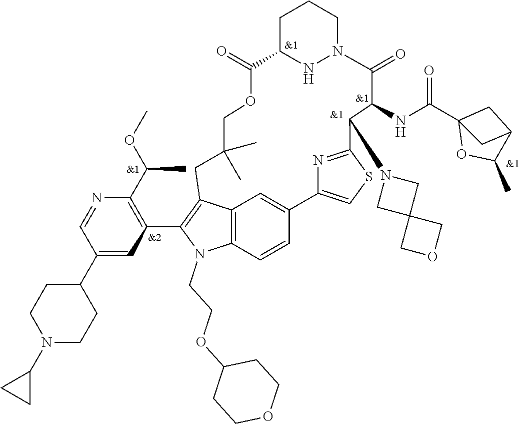 |
| A422 | 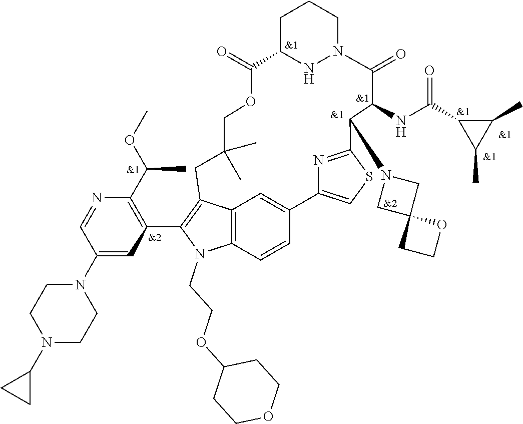 |
| A423 | 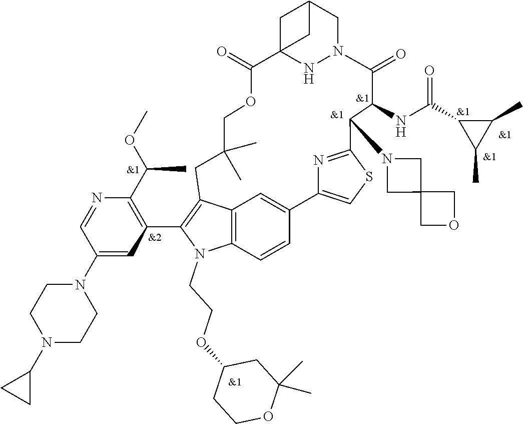 |
| A424 | 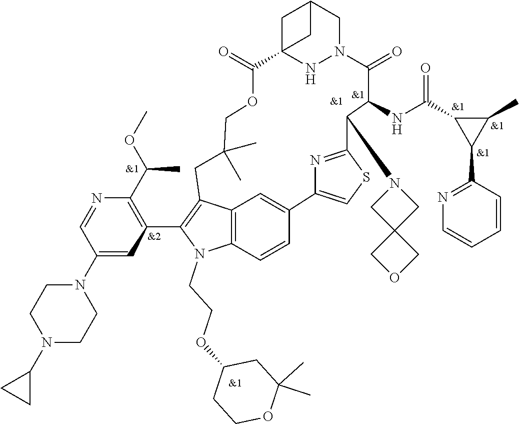 |
| A425 | 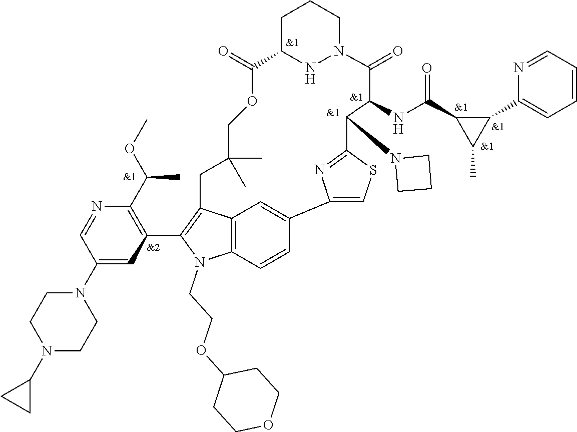 |
| A426 | 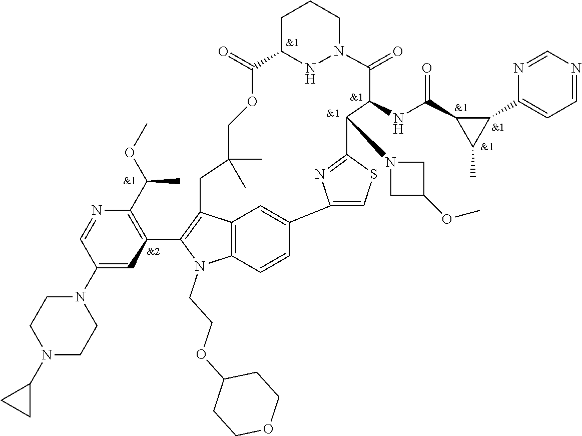 |
| A427 | 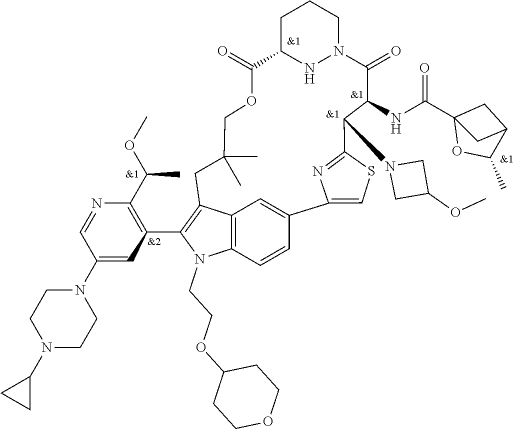 |
| A428 | 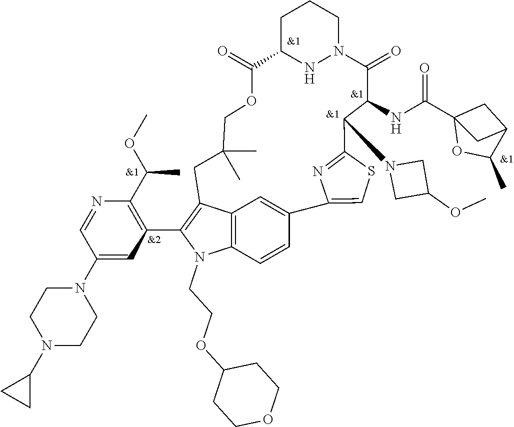 |
| A429 | 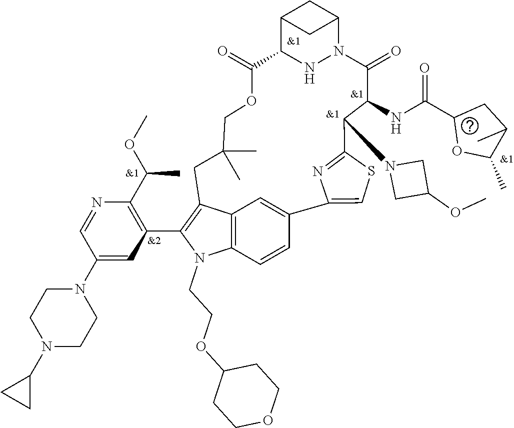 |
| A430 | 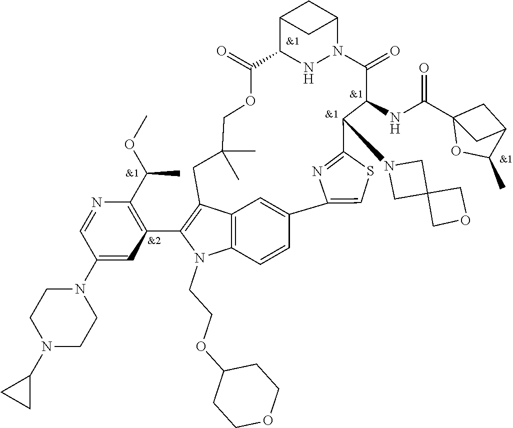 |
| A431 | 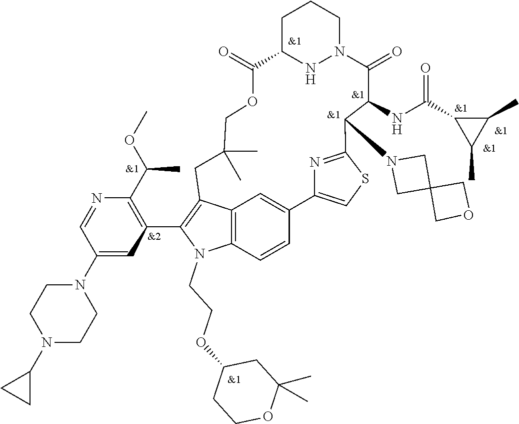 |
| A432 | 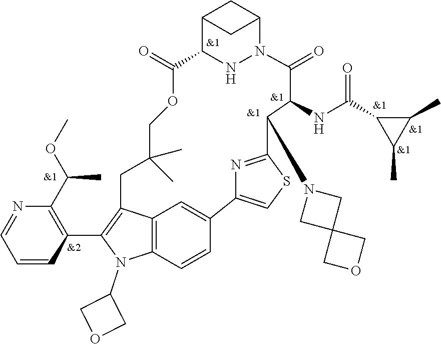 |
| A433 | 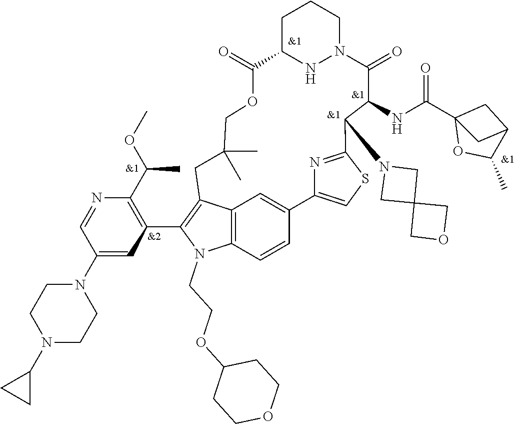 |
| A434 | 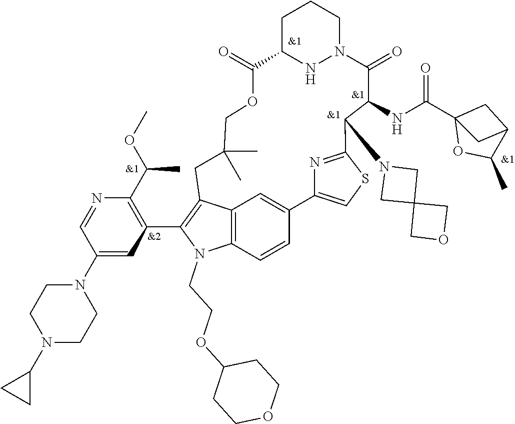 |
| A435 | 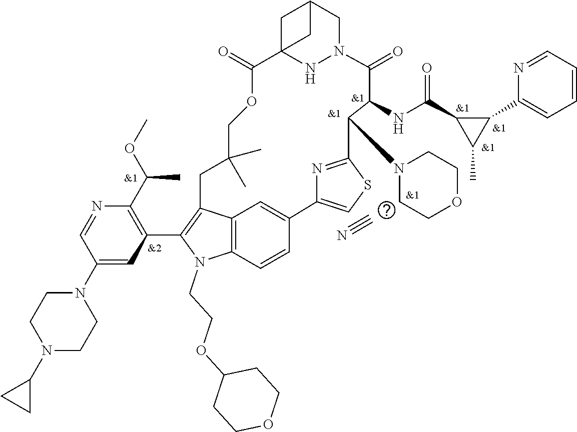 |
| A436 | 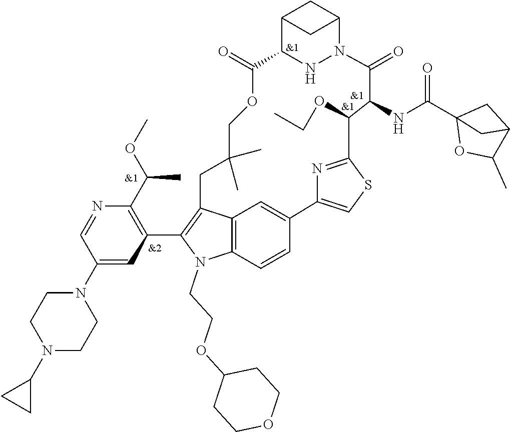 |
| A437 | 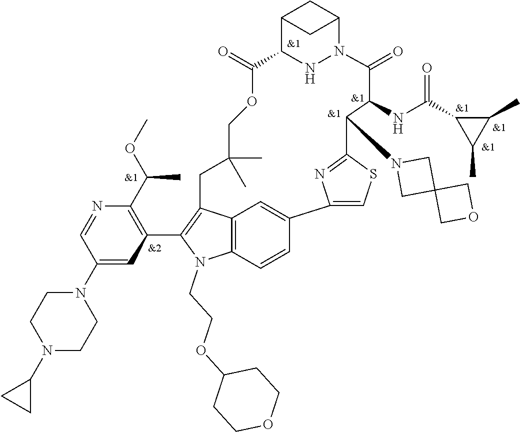 |
| A438 | 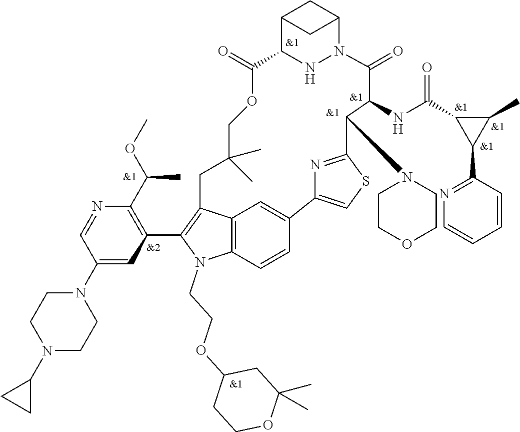 |
| A439 | 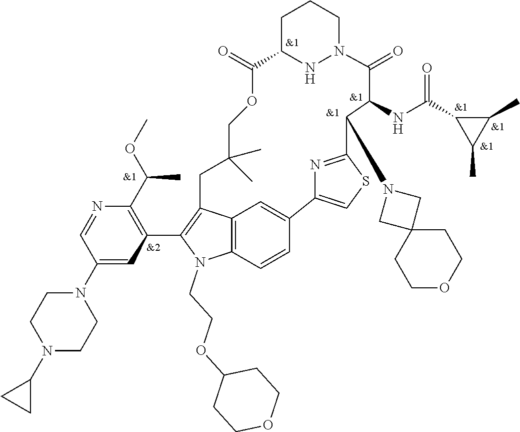 |
| A440 | 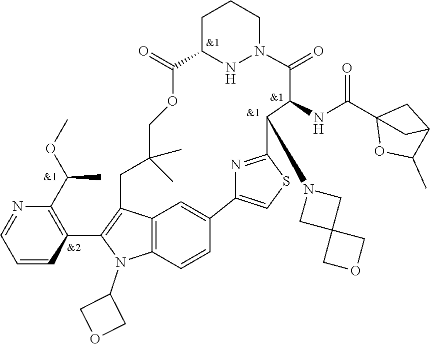 |
| A441 | 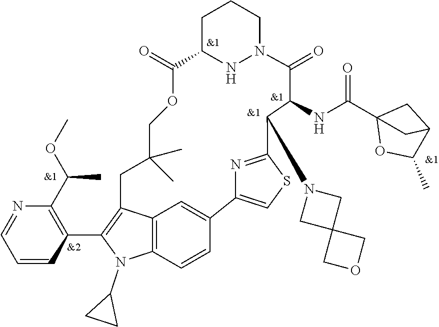 |
| A442 | 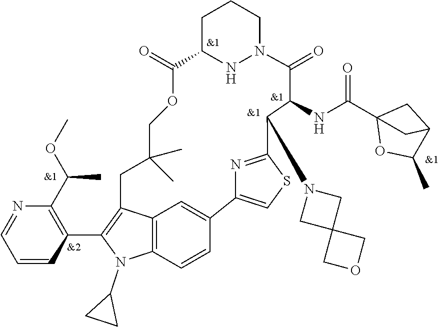 |
| A443 | 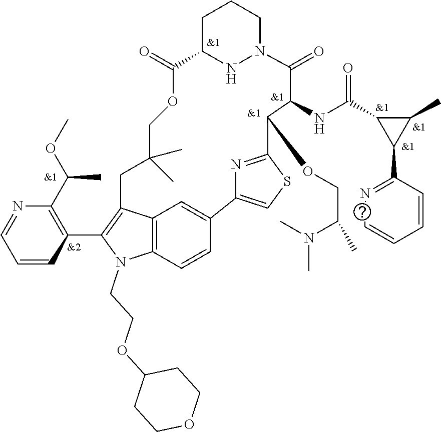 |
| A444 | 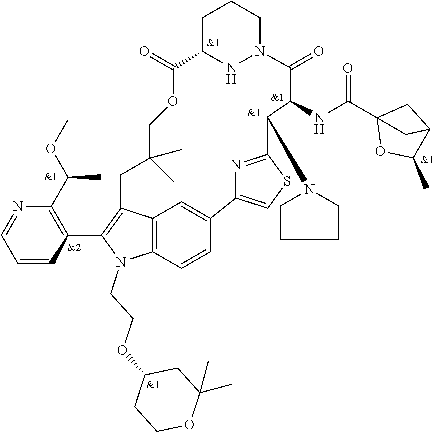 |
| A445 | 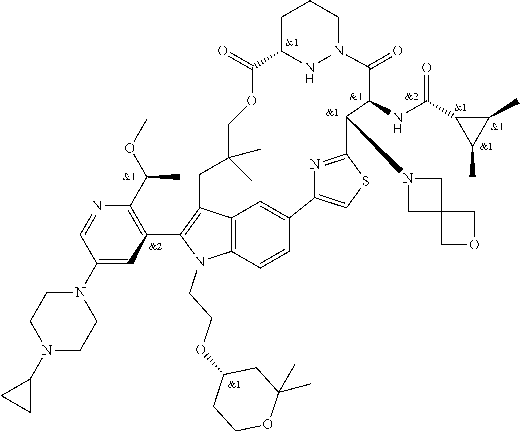 |
| A446 | 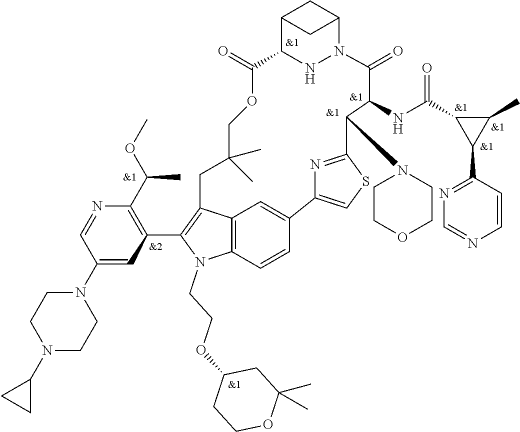 |
| A447 | 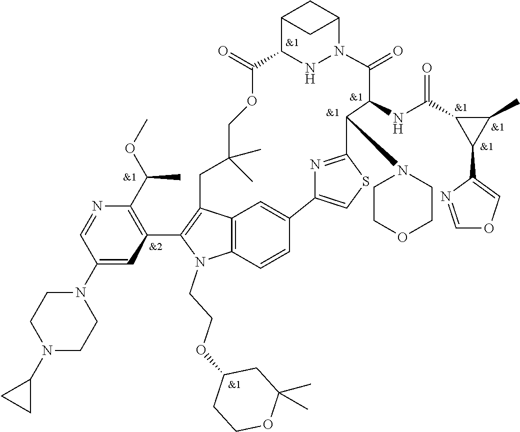 |
| A448 | 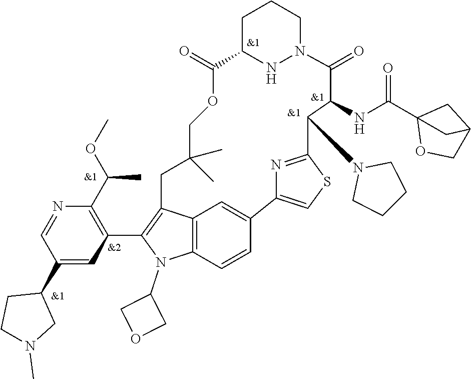 |
| A449 | 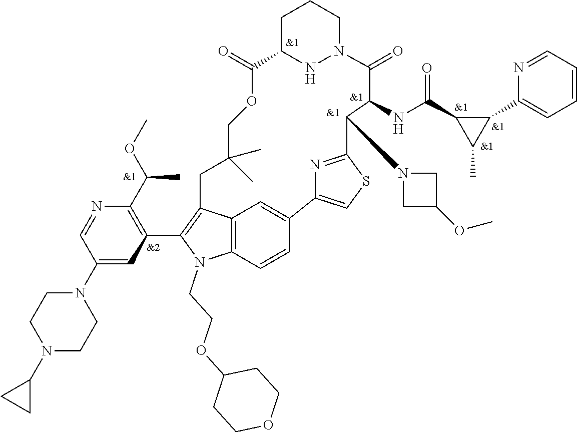 |
| A450 | 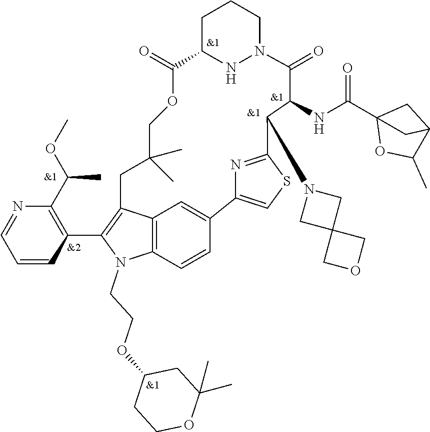 |
| A451 | 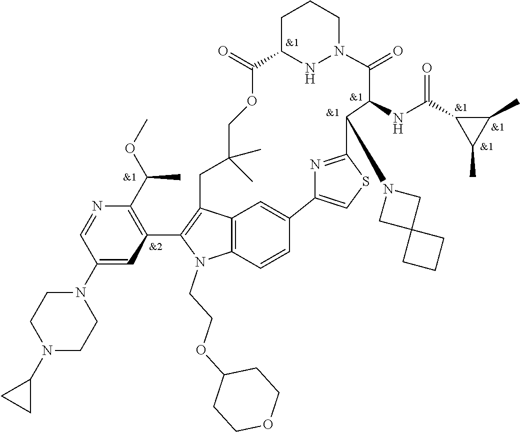 |
| A452 | 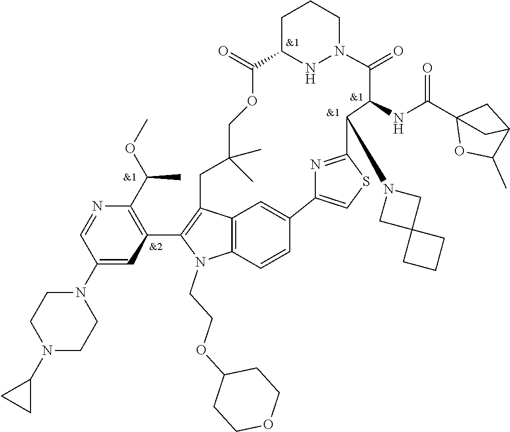 |
| A453 | 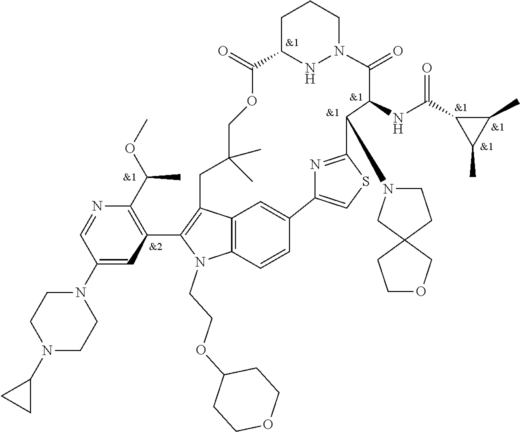 |
| A454 | 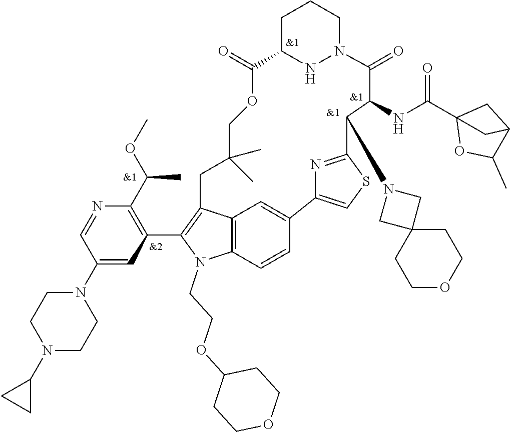 |
| A455 | 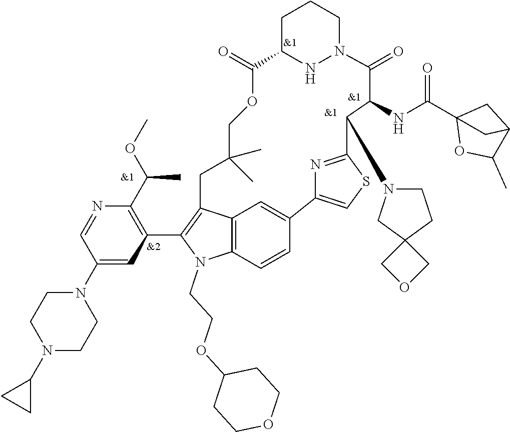 |
| A456 | 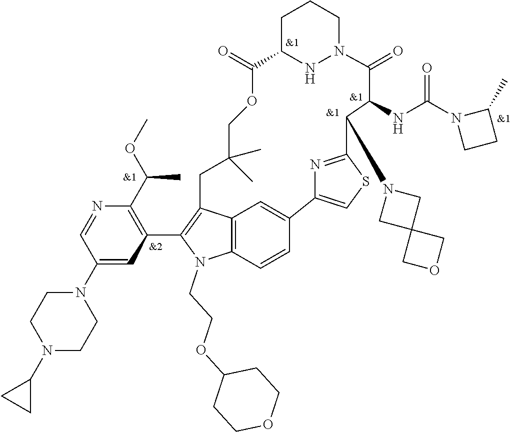 |
| A457 | 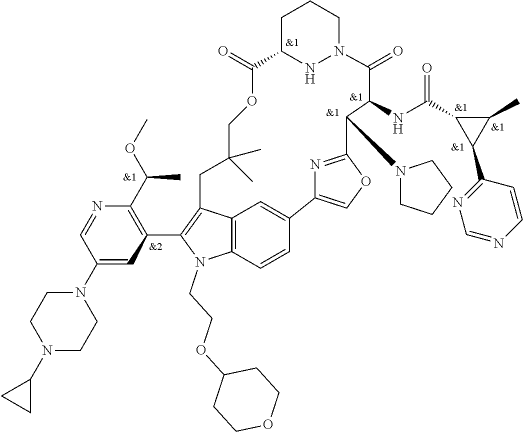 |
| A458 | 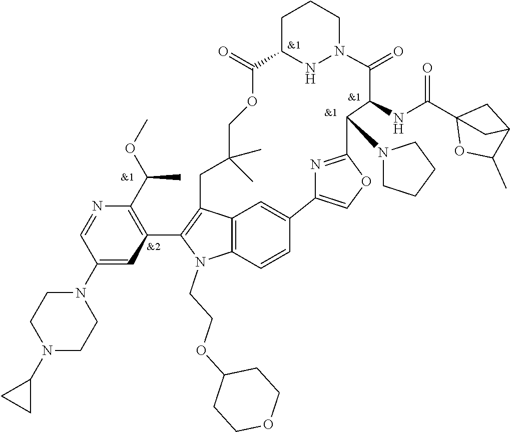 |
| A459 | 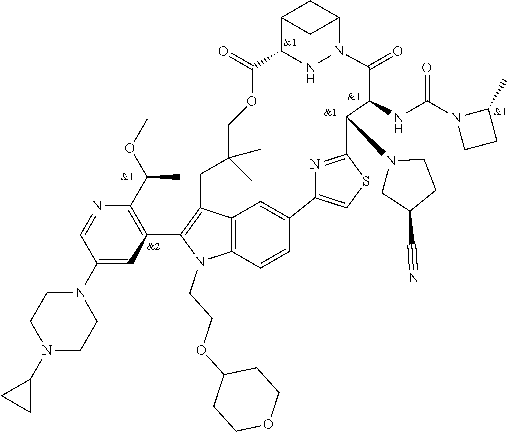 |
| A460 | 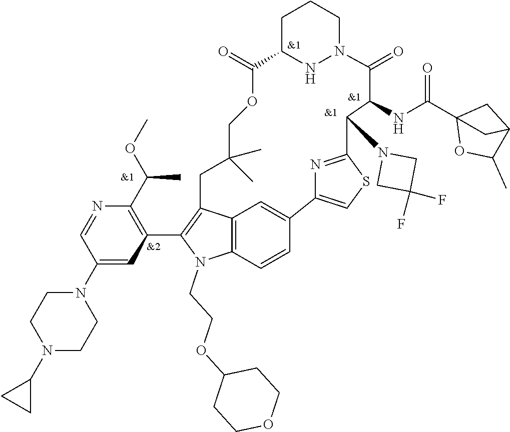 |
| A461 | 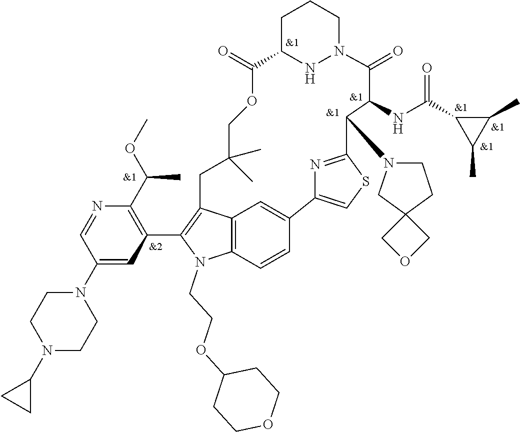 |
| A462 | 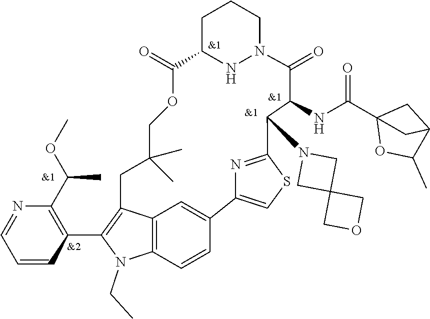 |
| A463 | 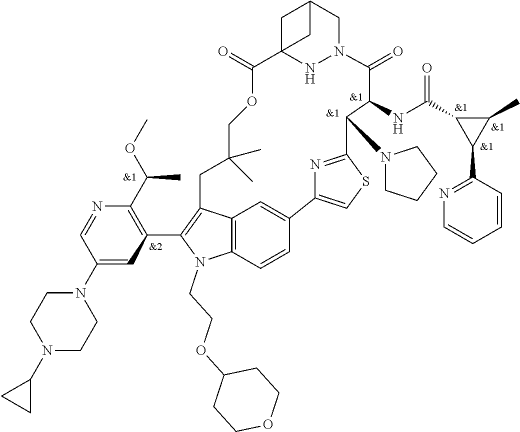 |
| A464 | 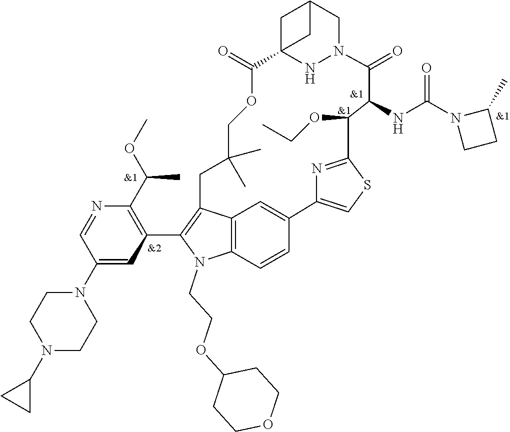 |
| A465 | 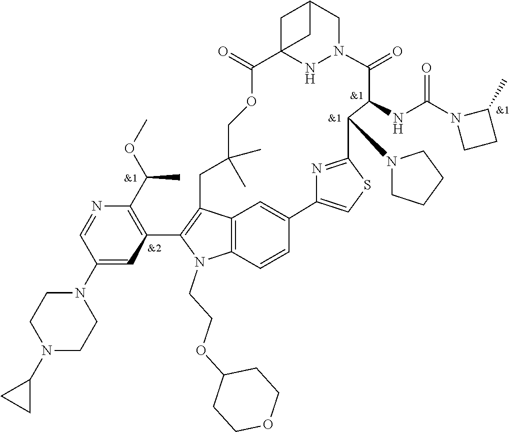 |
| A466 | 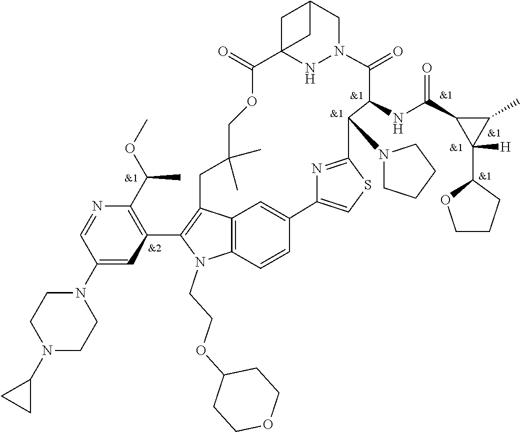 |
| A467 | 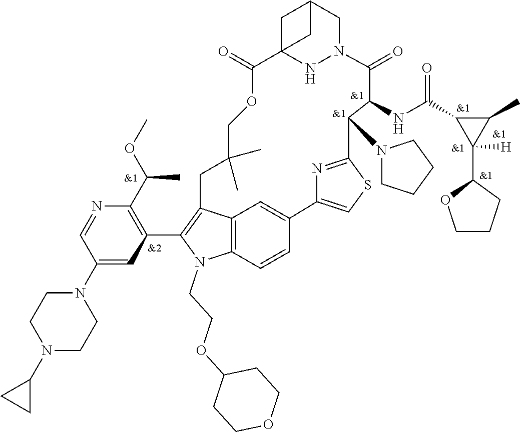 |
| A468 | 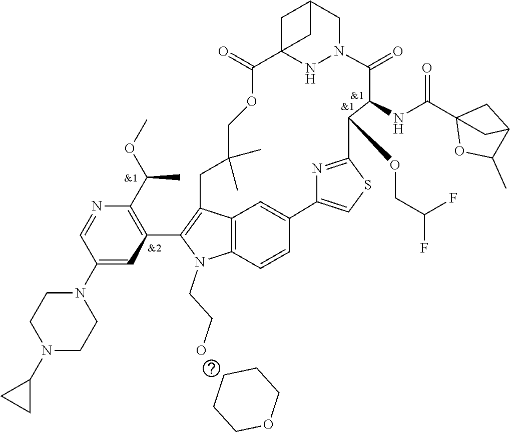 |
| A469 | 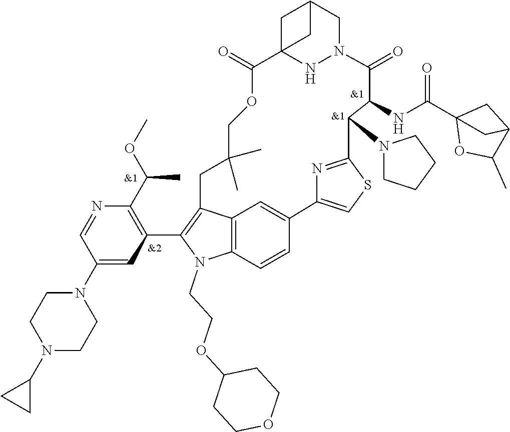 |
| A470 | 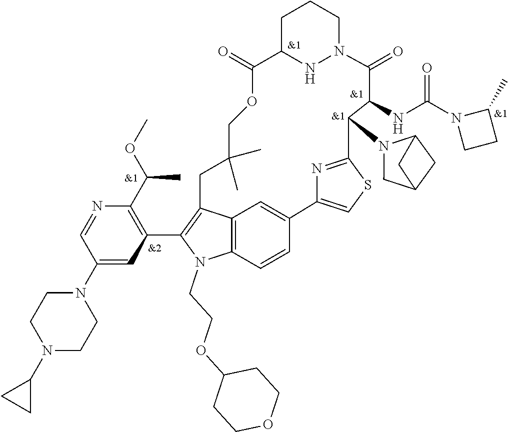 |
| A471 | 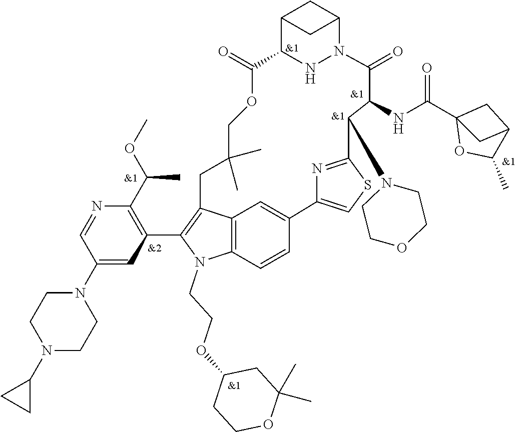 |
| A472 | 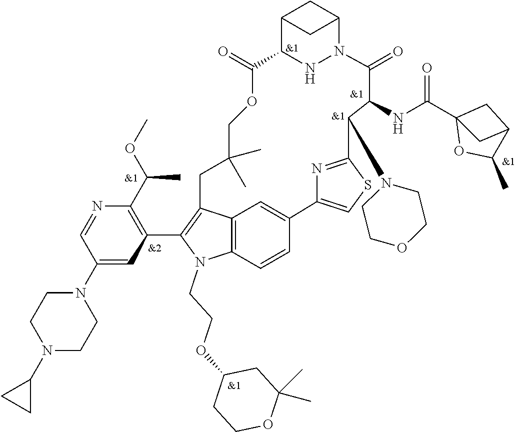 |
| A473 | 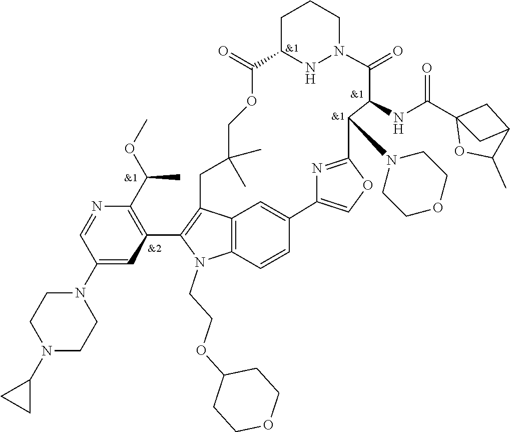 |
| A474 | 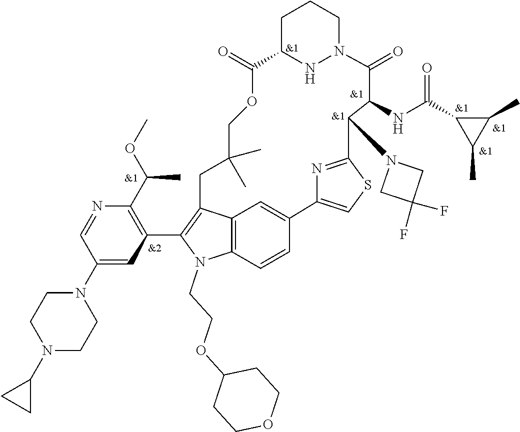 |
| A475 | 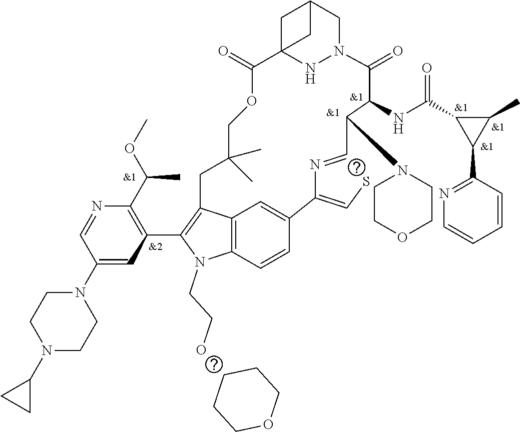 |
| A476 | 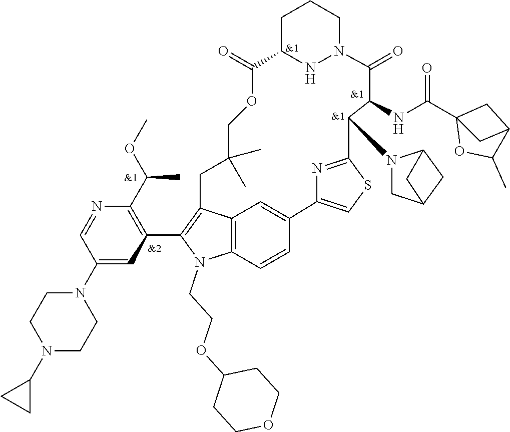 |
| A477 | 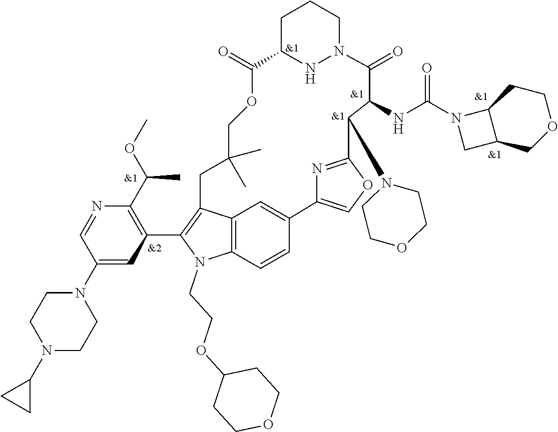 |
| A478 | 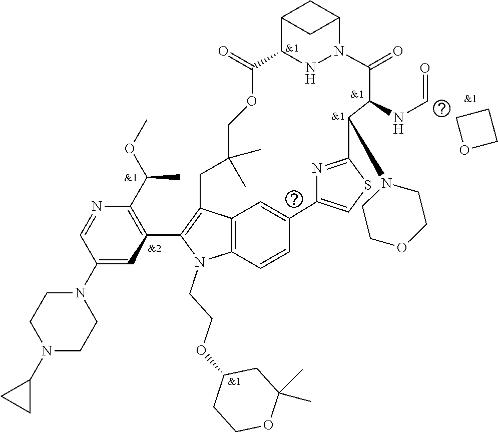 |
| A479 | 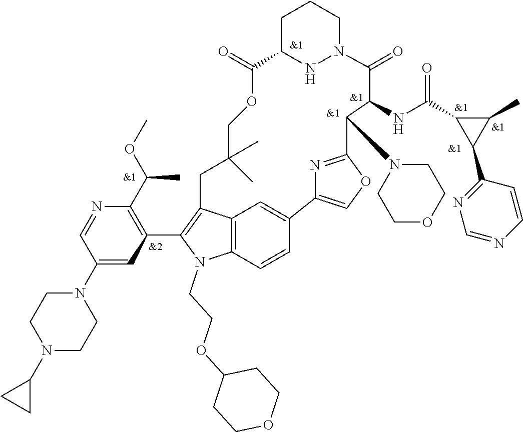 |
| A480 | 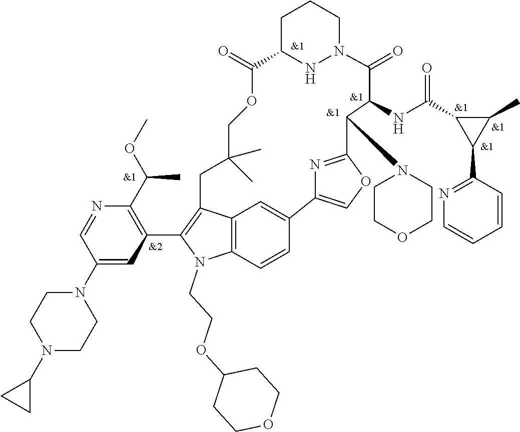 |
| A481 | 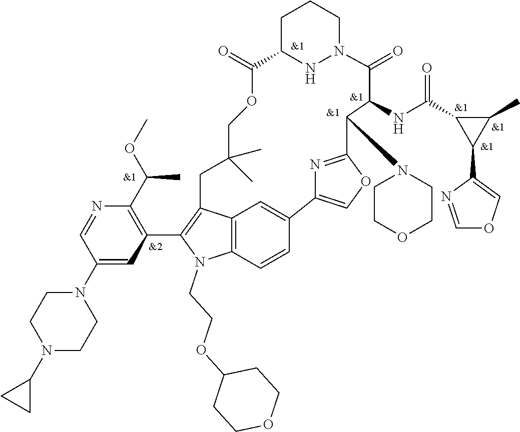 |
| A482 | 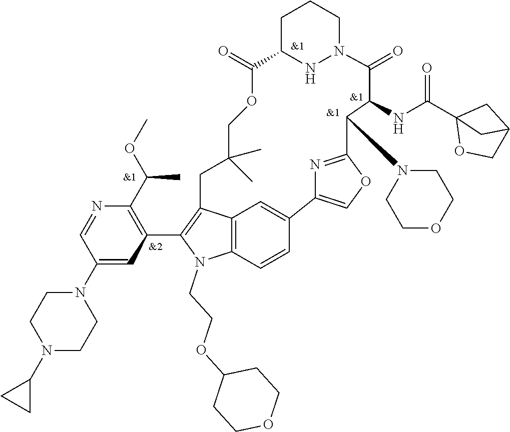 |
| A483 | 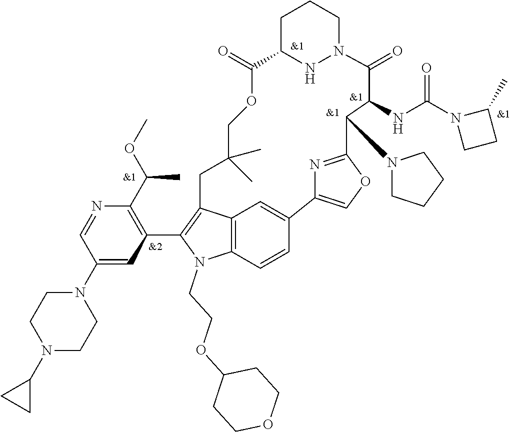 |
| A484 | 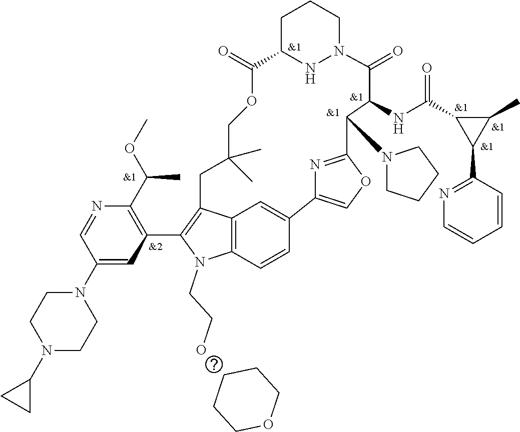 |
| A485 | 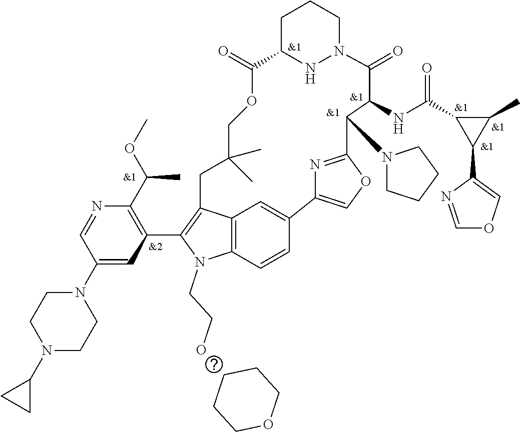 |
| A486 | 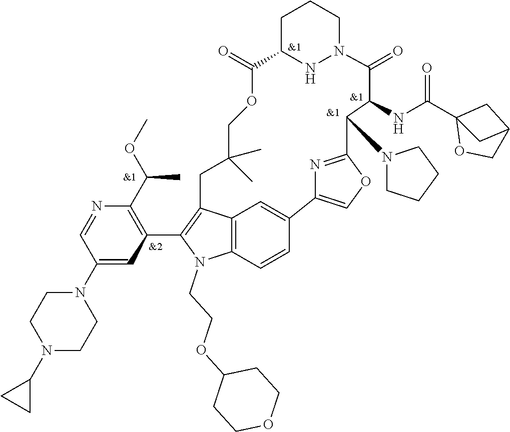 |
| A487 | 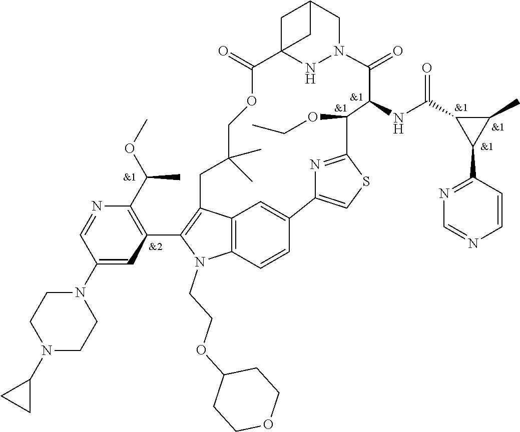 |
| A488 | 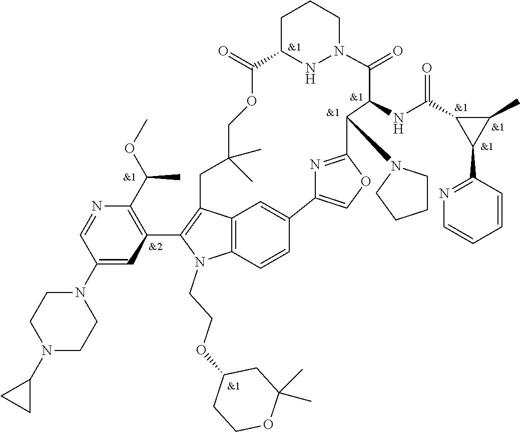 |
| A489 | 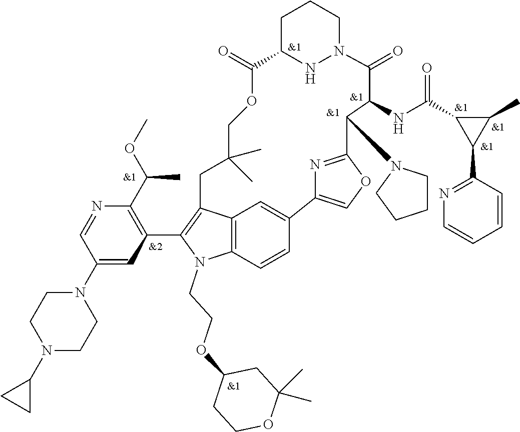 |
| A490 | 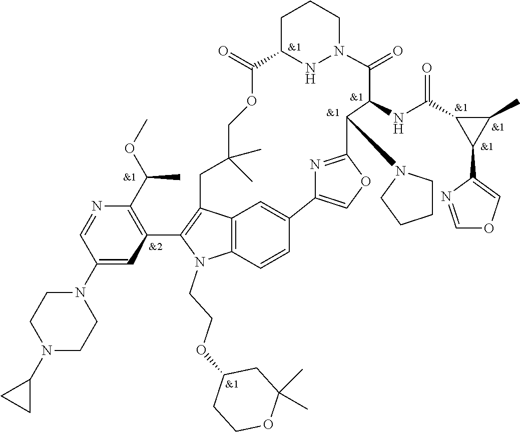 |
| A491 | 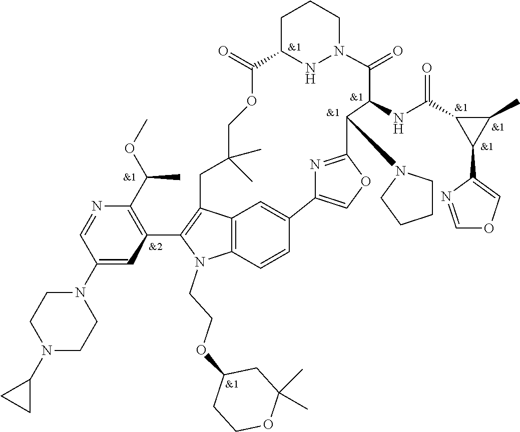 |
| A492 | 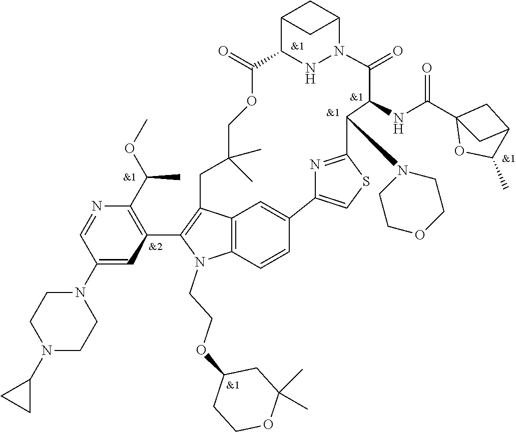 |
| A493 | 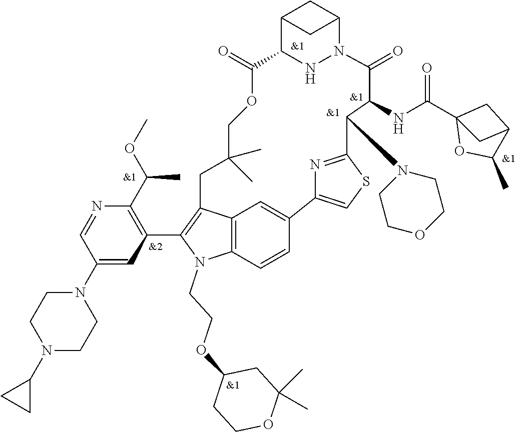 |
| A494 | 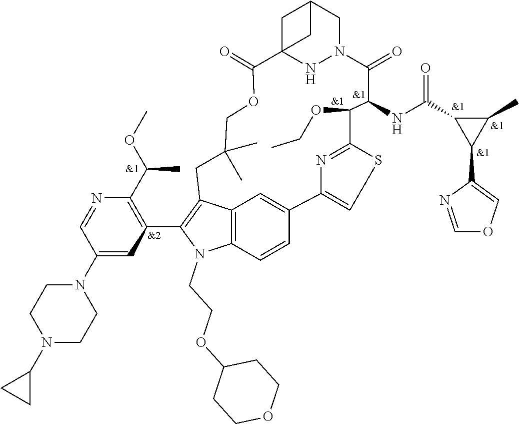 |
| A495 | 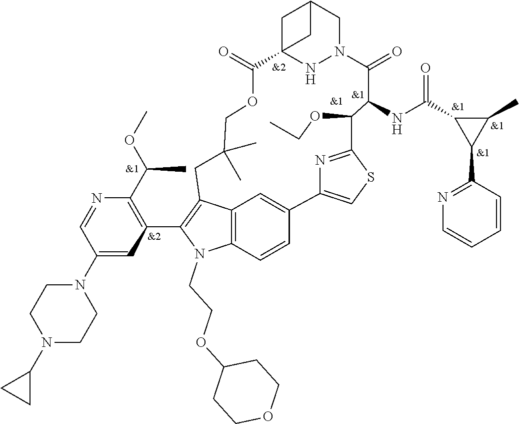 |
| A496 | 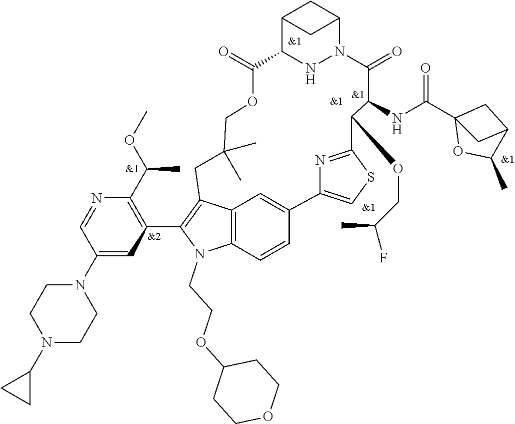 |
| A497 | 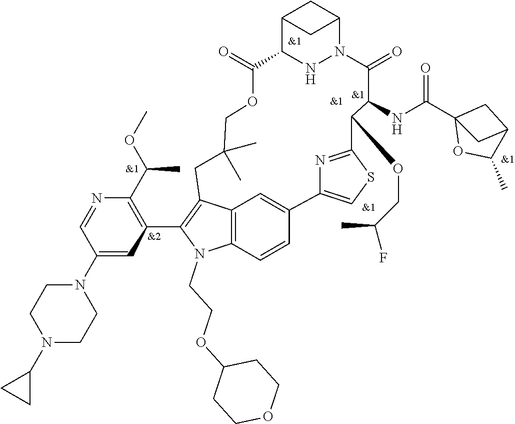 |
| A498 | 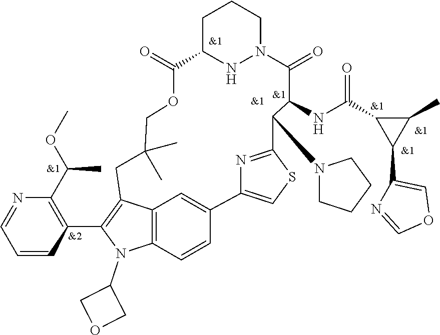 |
| A499 | 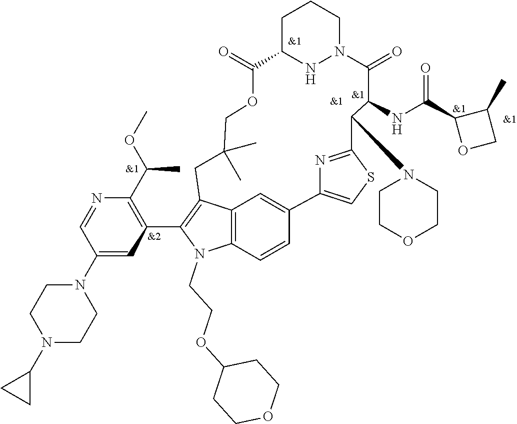 |
| A500 | 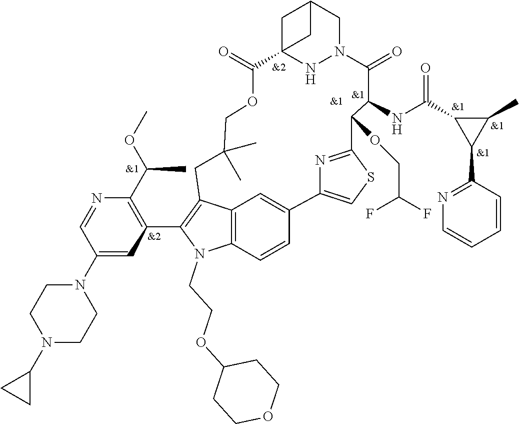 |
| A501 | 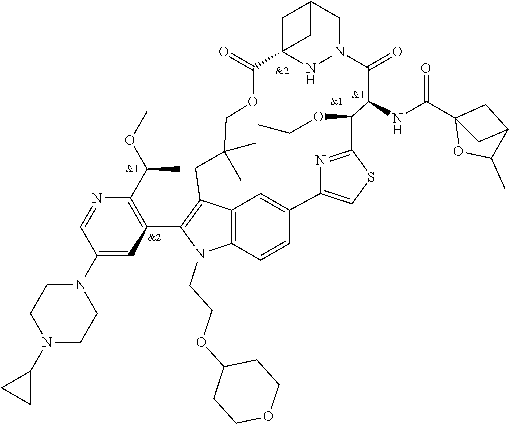 |
| A502 | 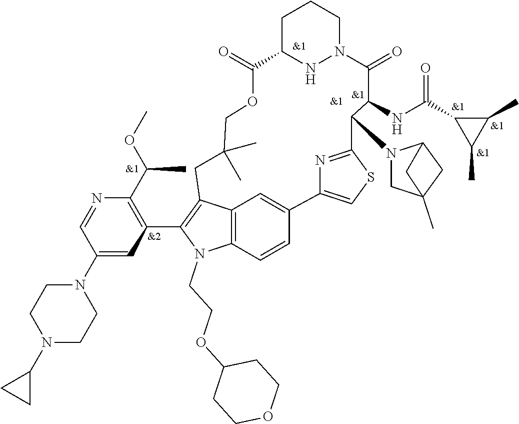 |
| A503 | 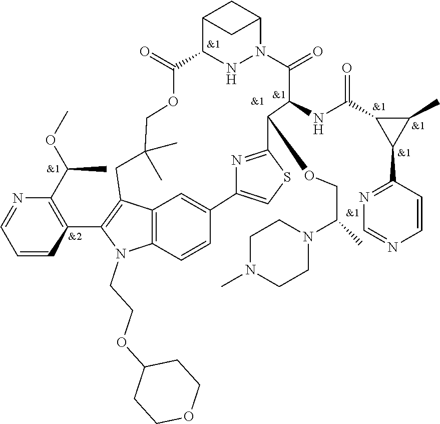 |
| A504 | 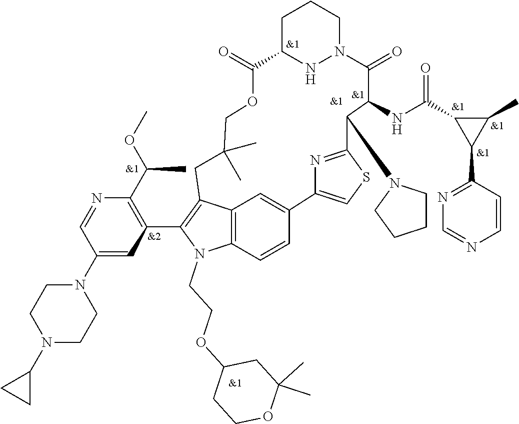 |
| A505 | 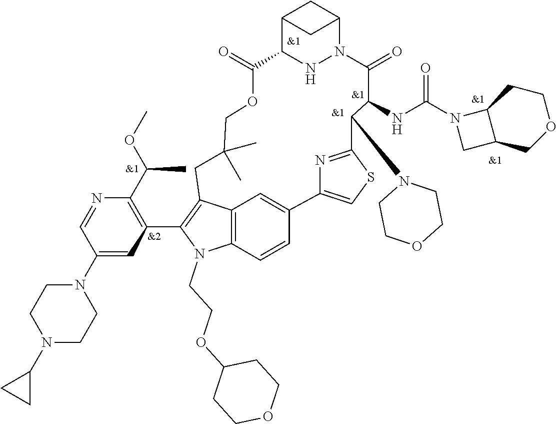 |
| A506 | 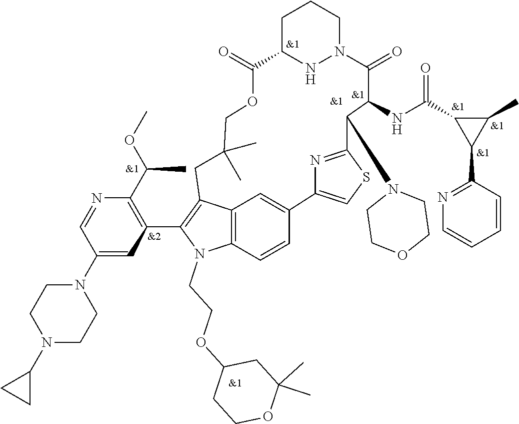 |
| A507 | 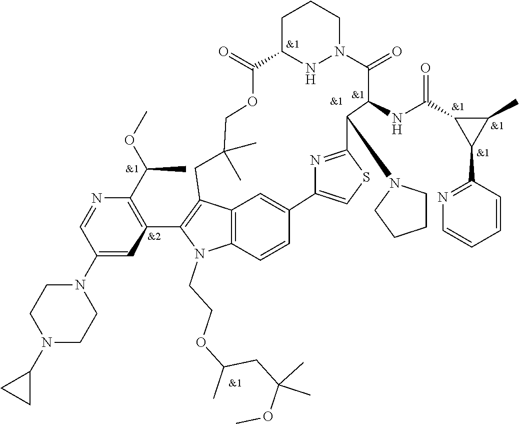 |
| A508 | 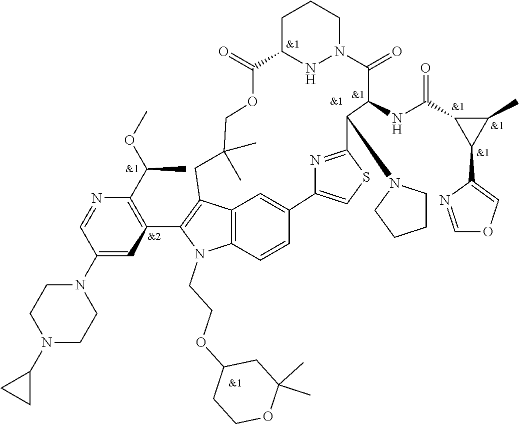 |
| A509 | 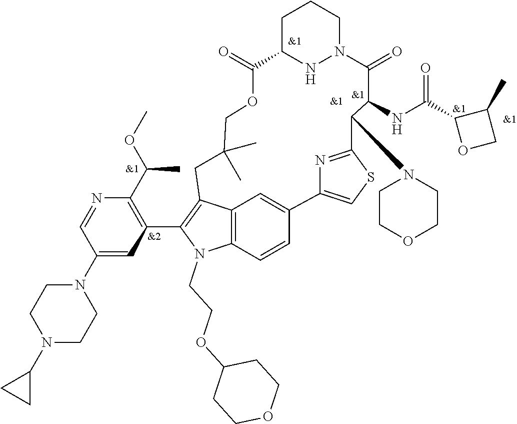 |
| A510 | 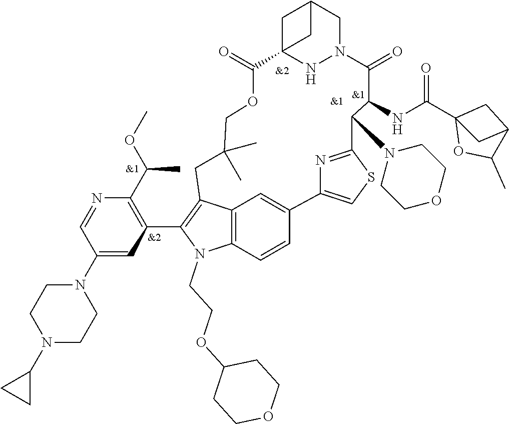 |
| A511 | 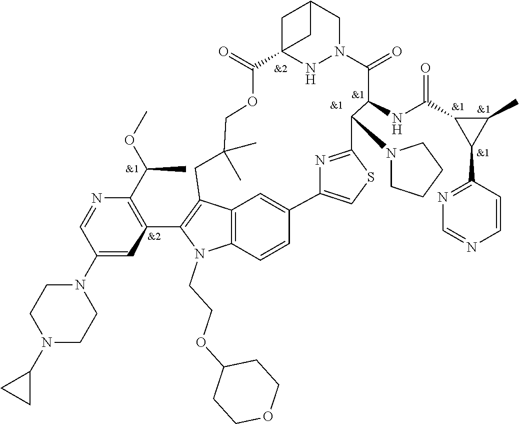 |
| A512 | 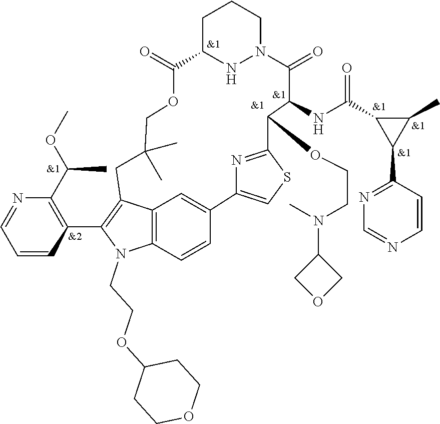 |
| A513 | 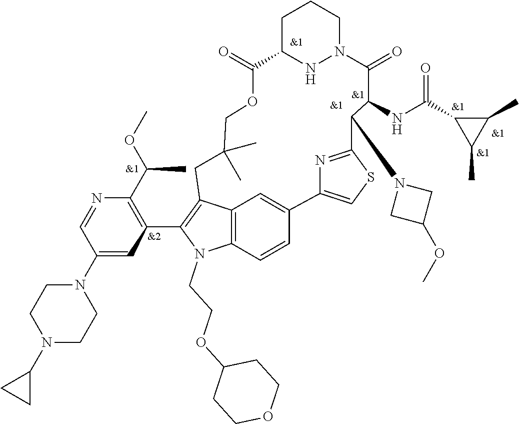 |
| A514 | 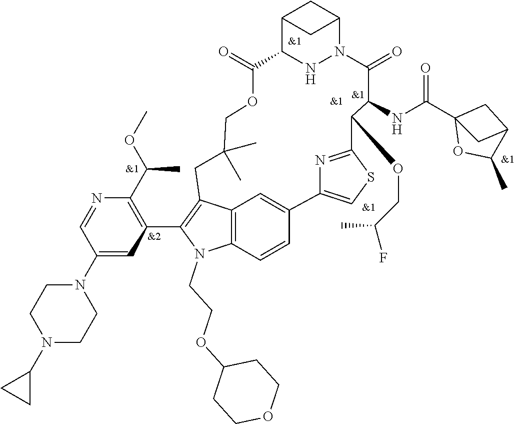 |
| A515 | 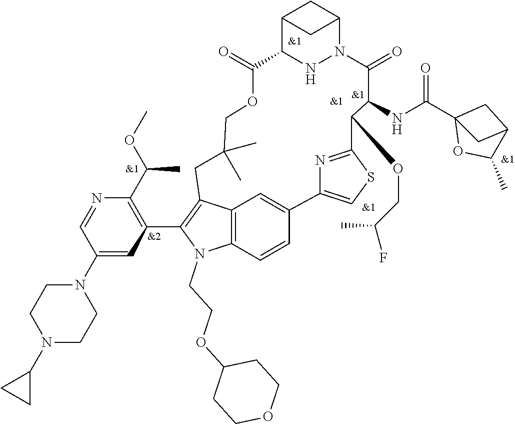 |
| A516 | 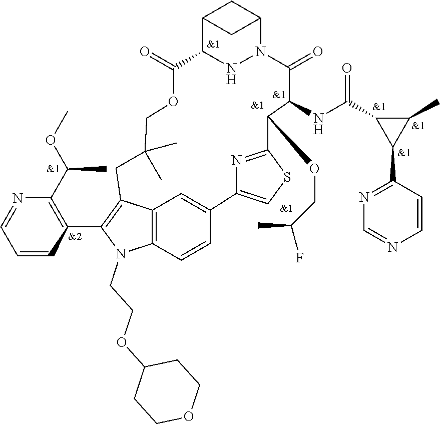 |
| A517 | 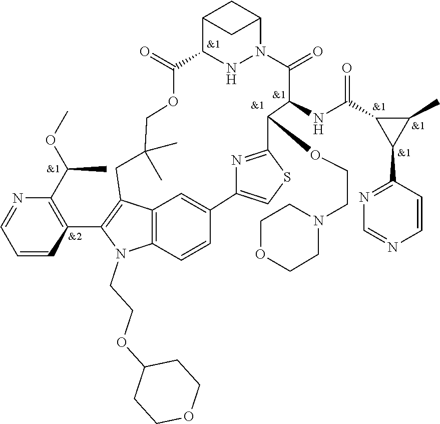 |
| A518 | 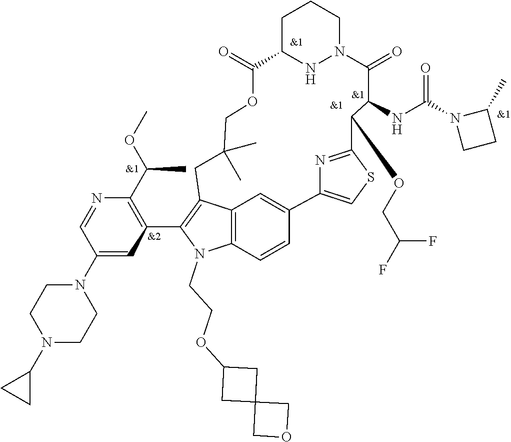 |
| A519 | 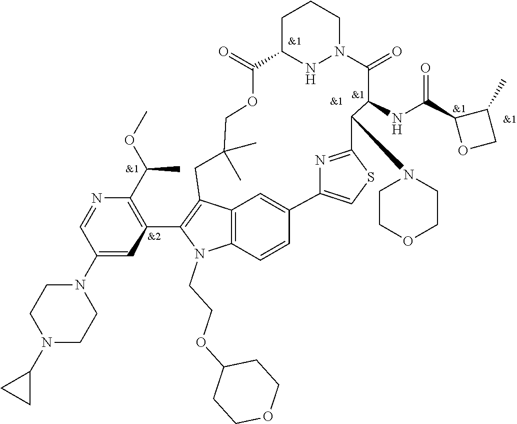 |
| A520 | 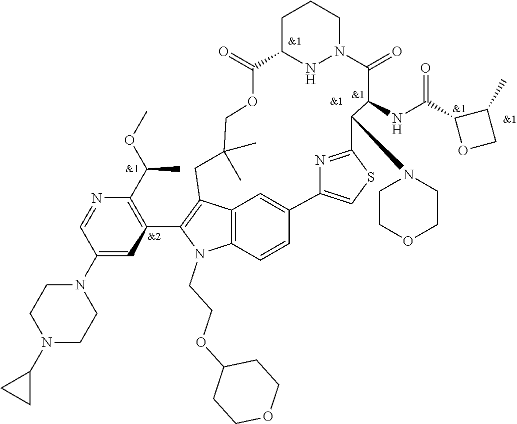 |
| A521 | 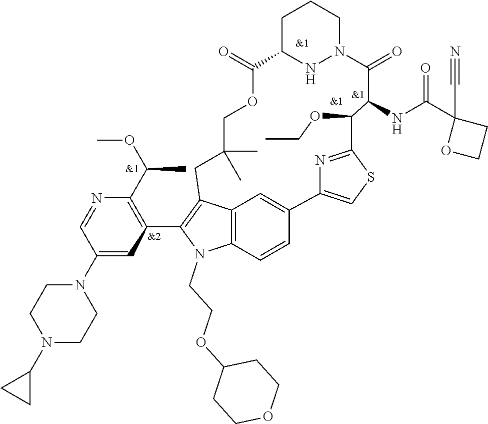 |
| A522 | 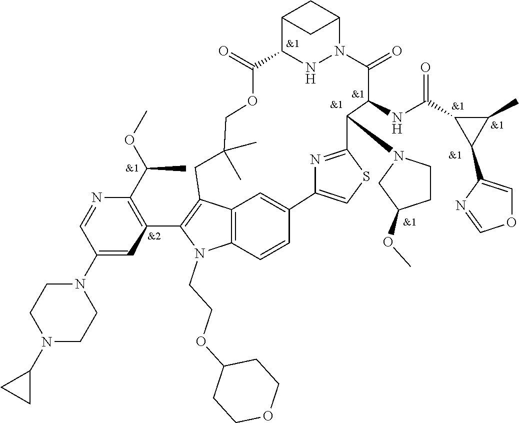 |
| A523 | 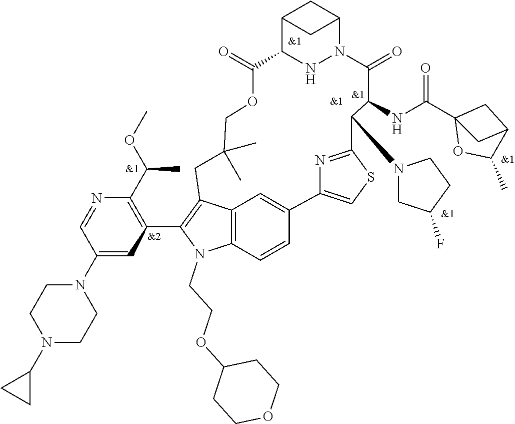 |
| A524 | 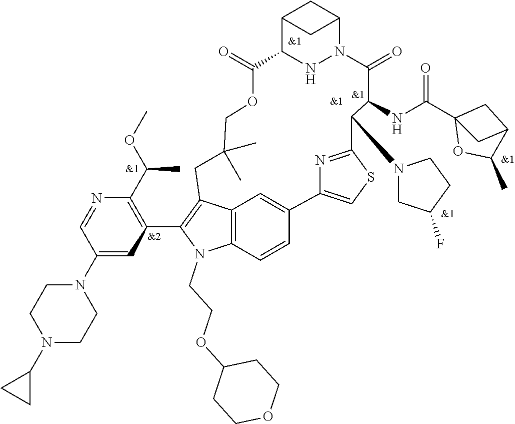 |
| A525 | 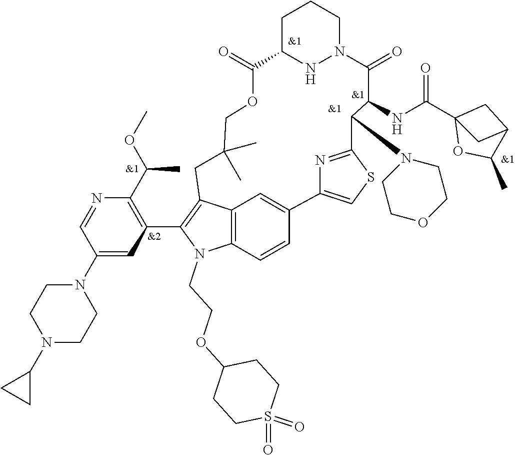 |
| A526 | 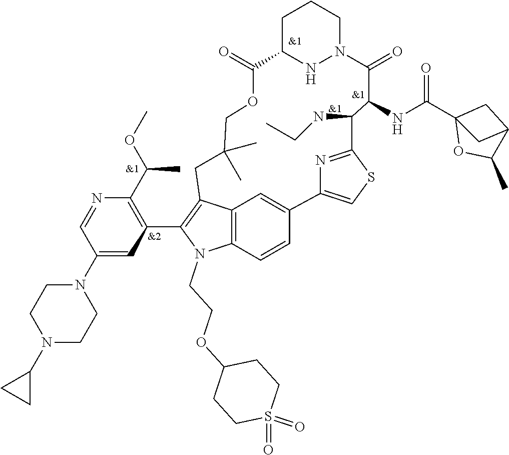 |
| A527 | 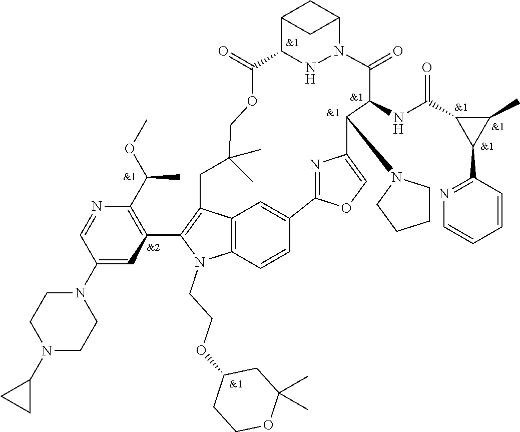 |
| A528 | 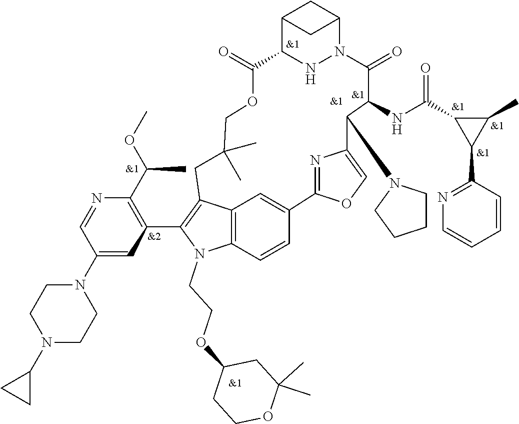 |
| A529 | 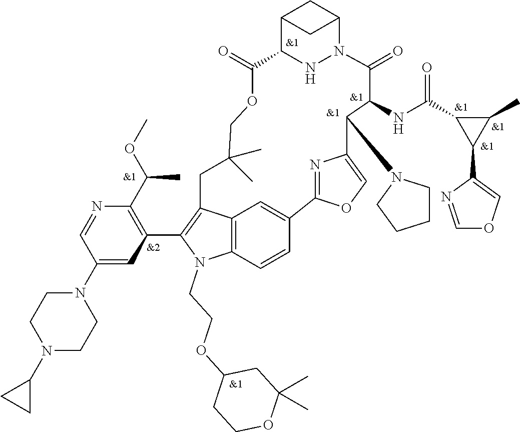 |
| A530 | 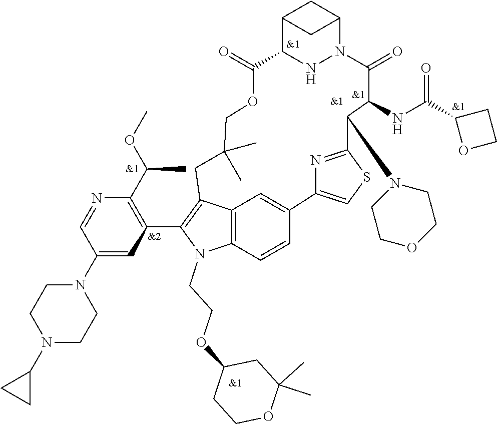 |
| A531 | 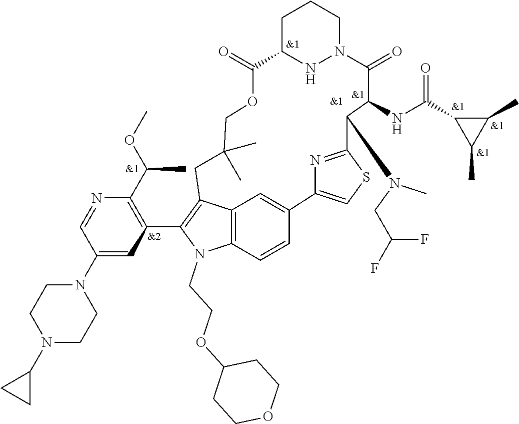 |
| A532 | 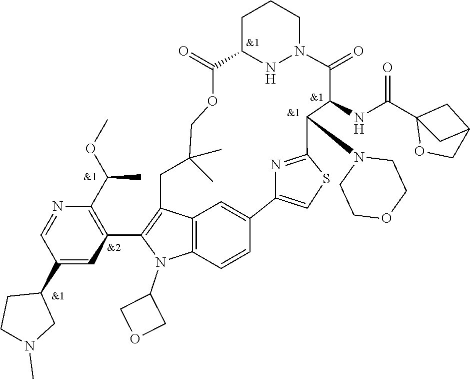 |
| A533 | 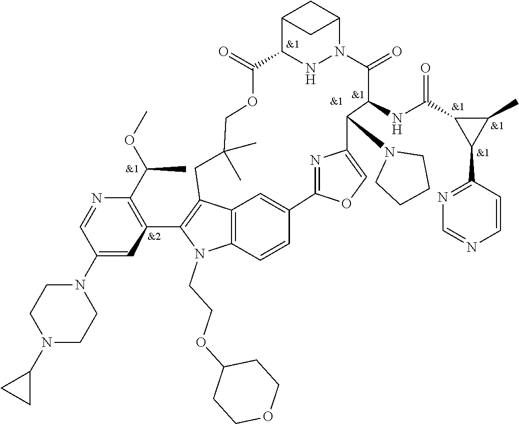 |
| A534 | 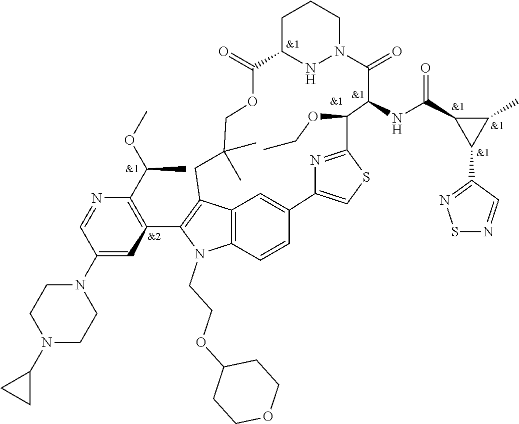 |
| A535 | 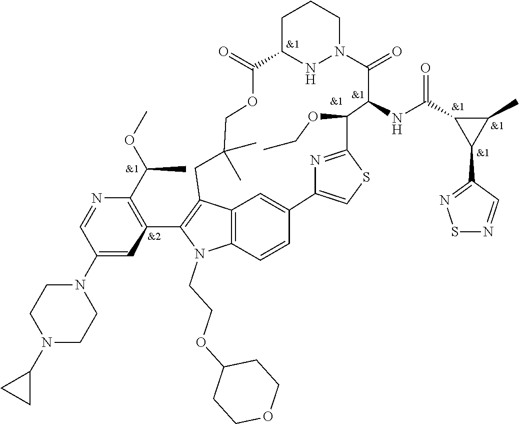 |
| A536 | 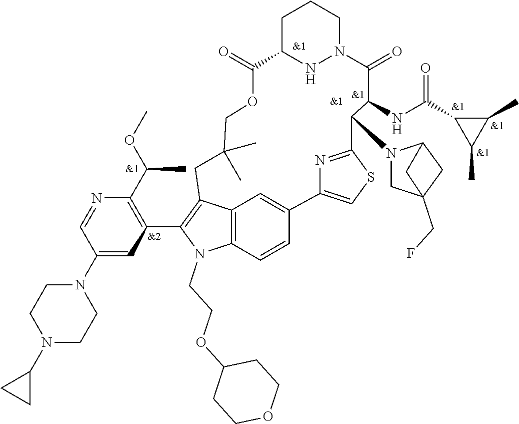 |
| A537 | 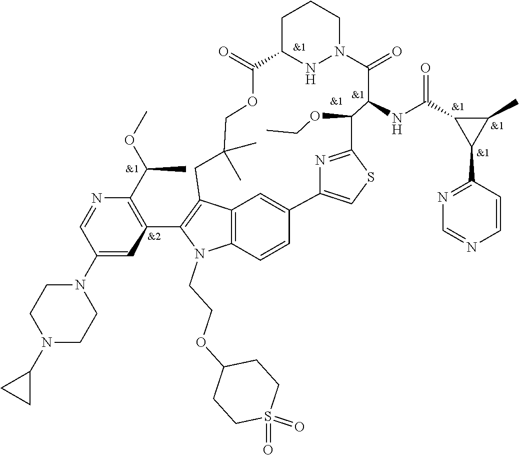 |
| A538 | 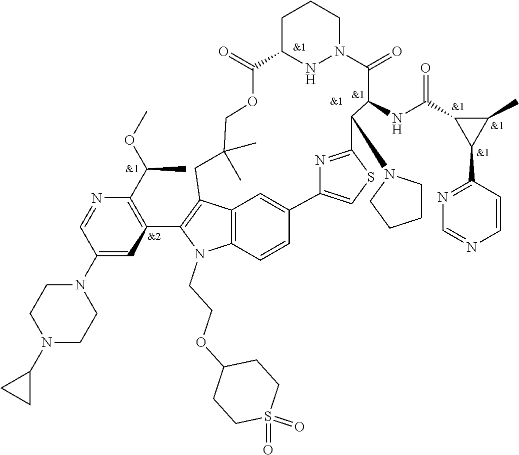 |
| A539 | 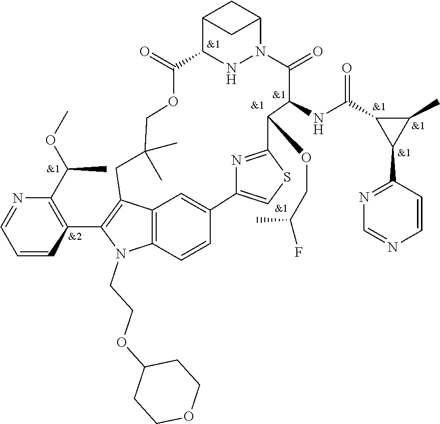 |
| A540 | 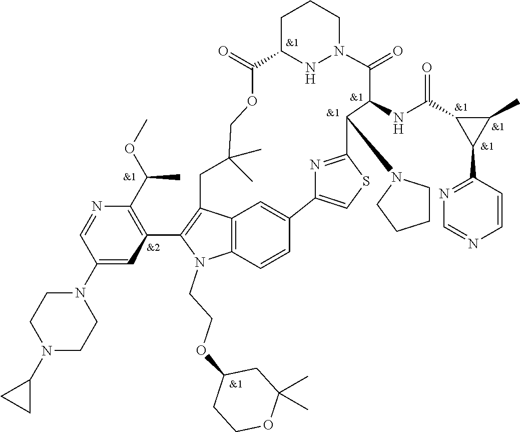 |
| A541 | 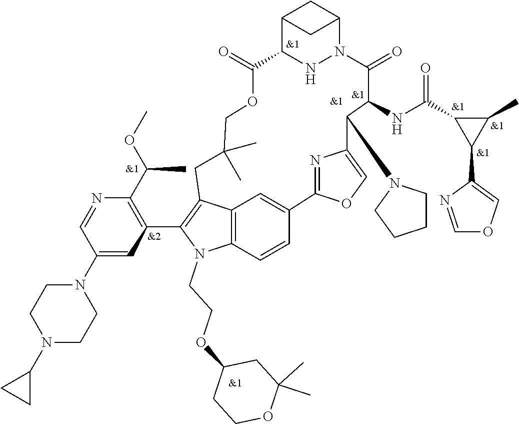 |
| A542 | 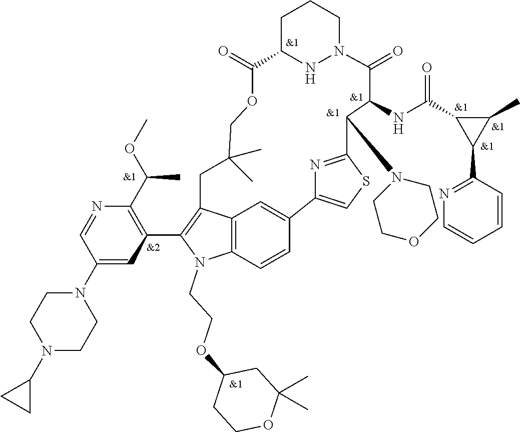 |
| A543 | 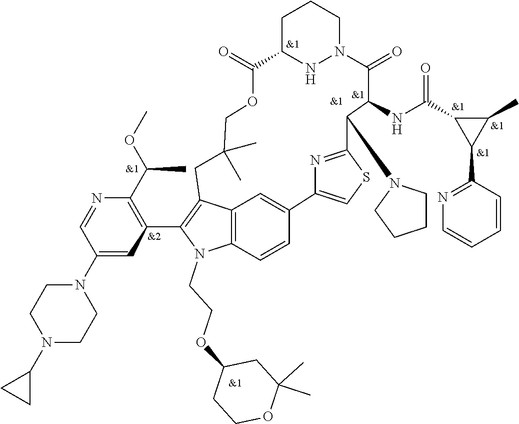 |
| A544 | 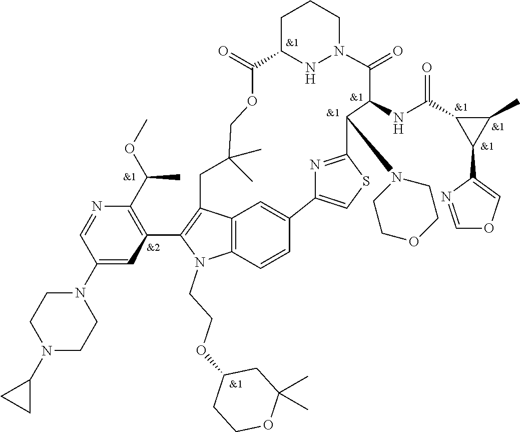 |
| A545 | 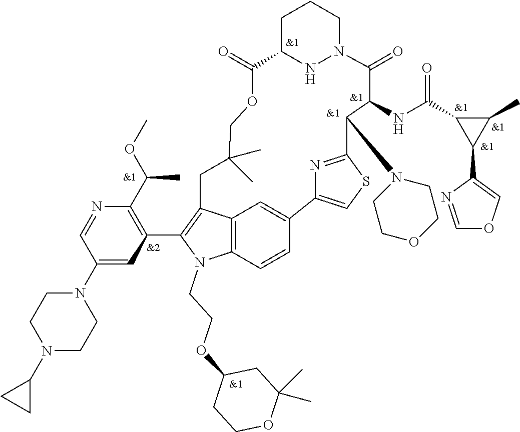 |
| A546 | 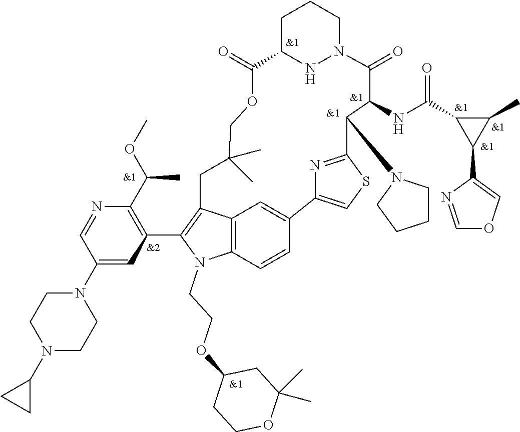 |
| A547 | 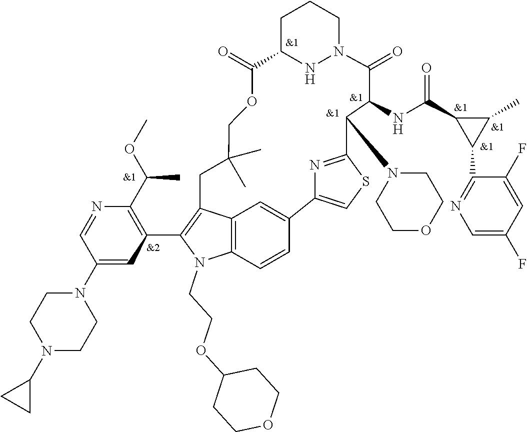 |
| A548 | 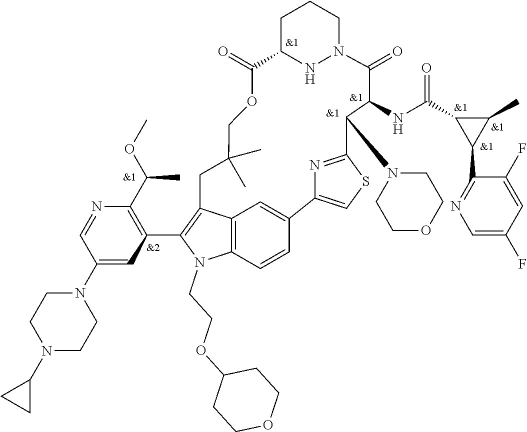 |
| A549 | 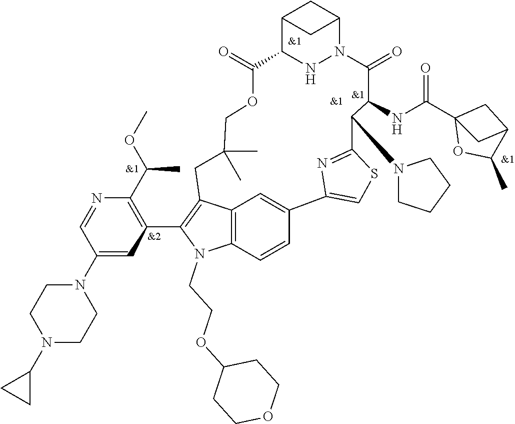 |
| A550 | 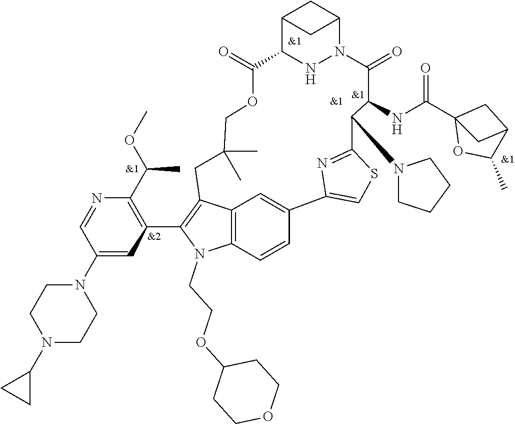 |
| A551 | 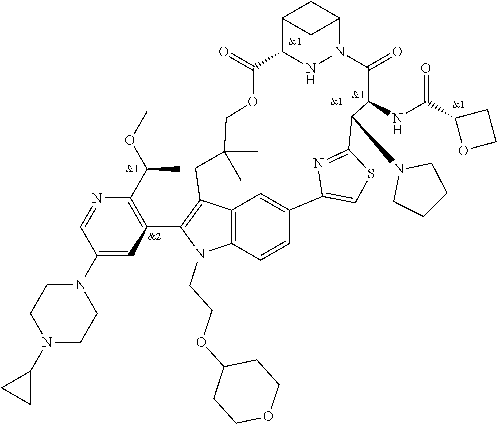 |
| A552 | 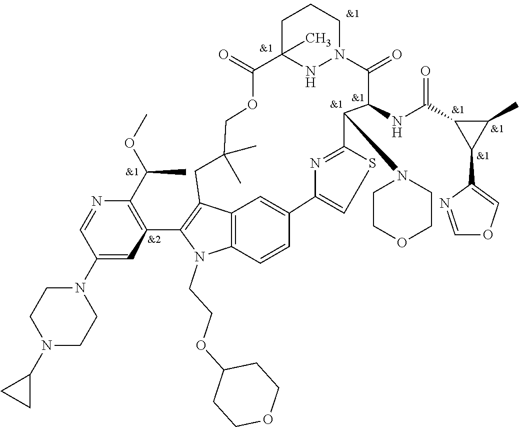 |
| A553 | 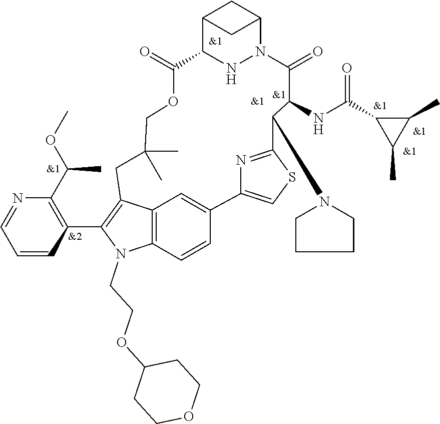 |
| A554 | 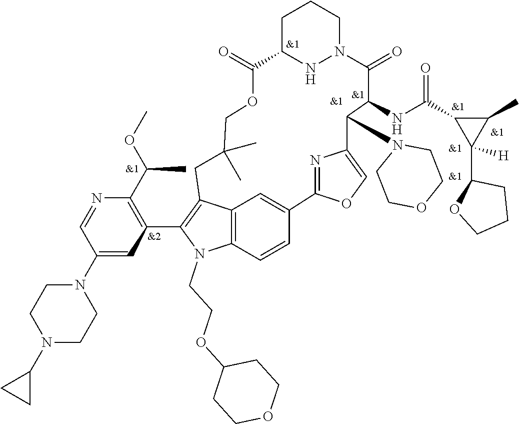 |
| A555 | 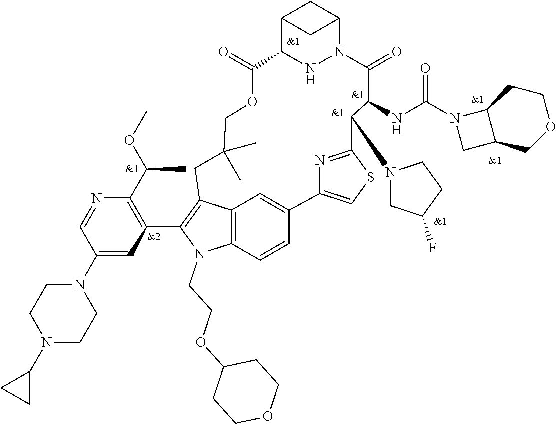 |
| A556 | 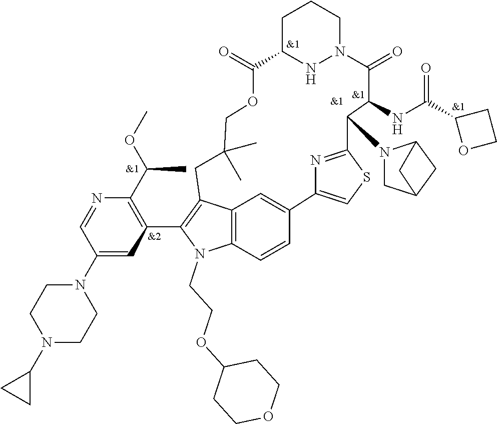 |
| A557 | 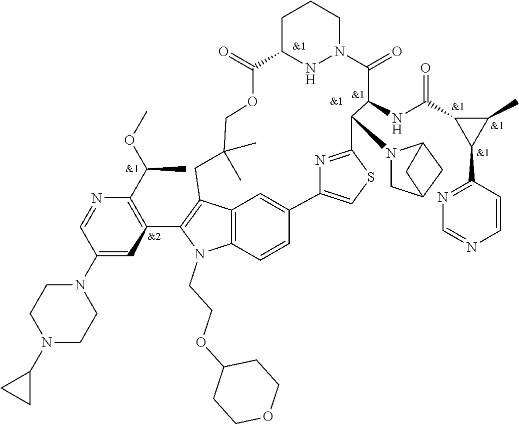 |
| A558 | 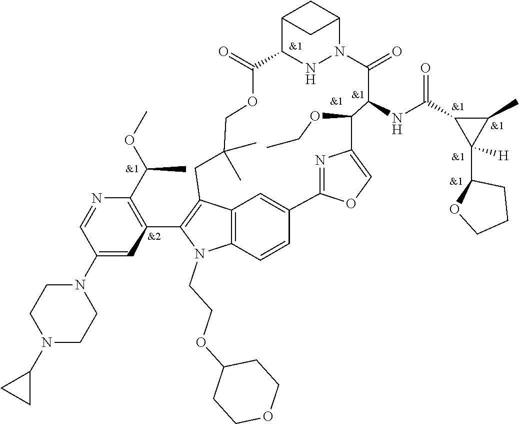 |
| A559 | 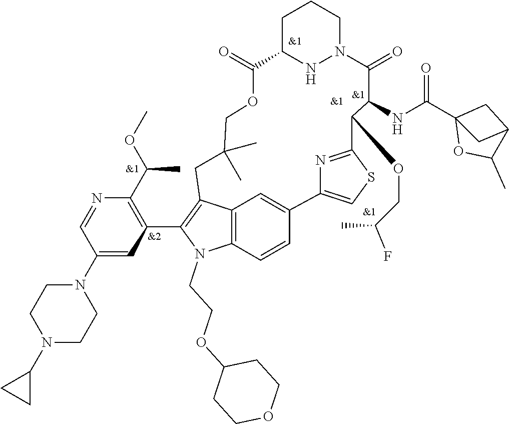 |
| A560 | 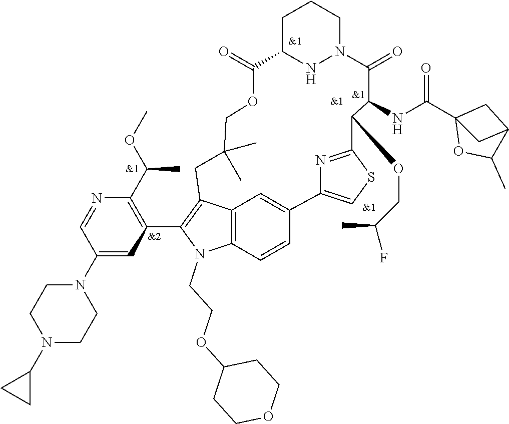 |
| A561 | 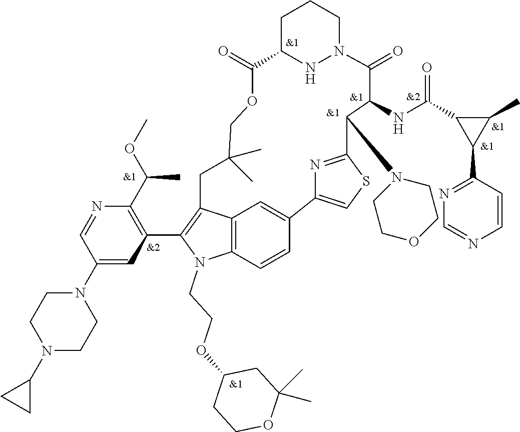 |
| A562 | 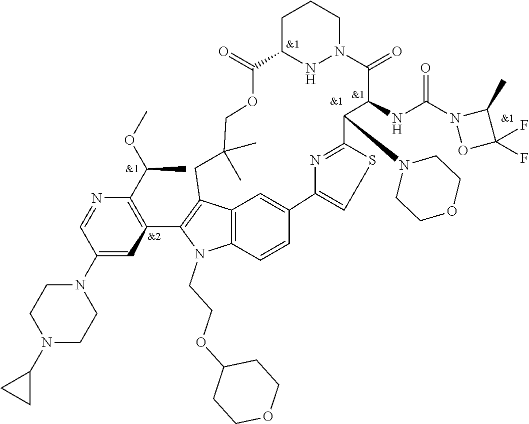 |
| A563 | 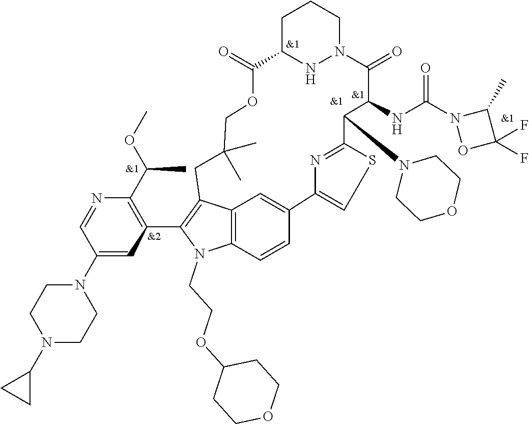 |
| A564 | 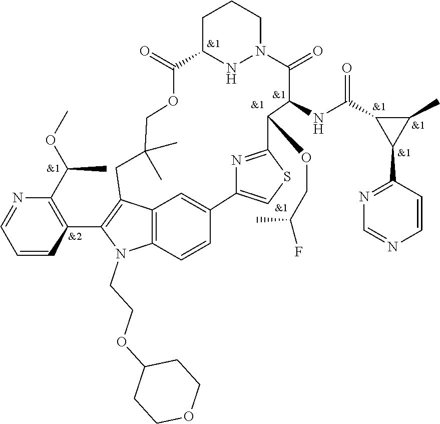 |
| A565 | 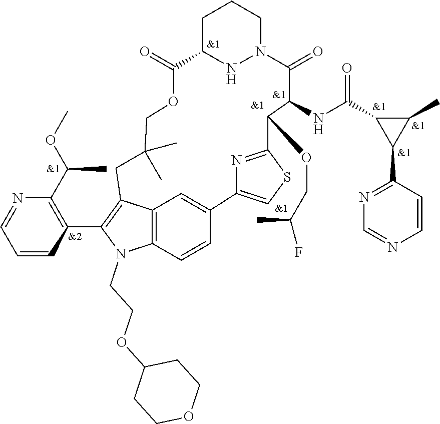 |
| A566 | 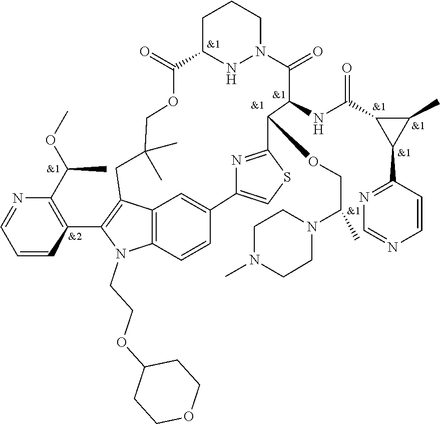 |
| A567 | 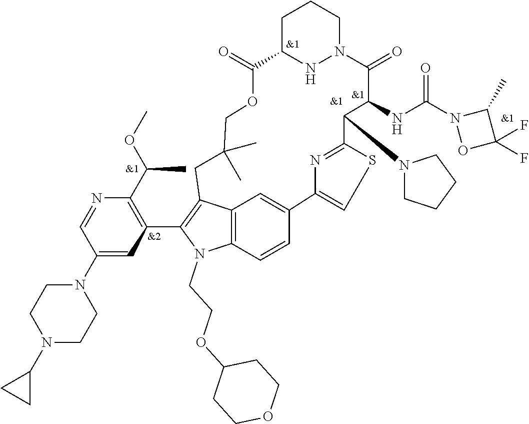 |
| A568 | 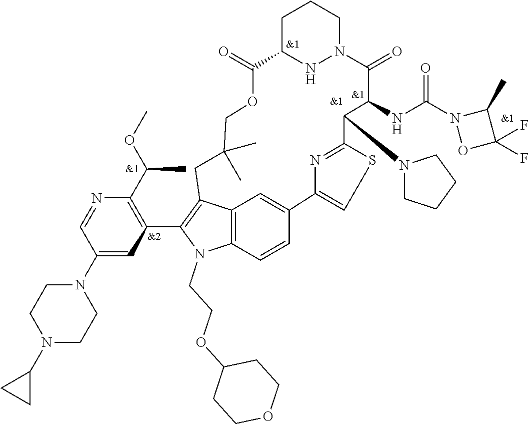 |
| A569 | 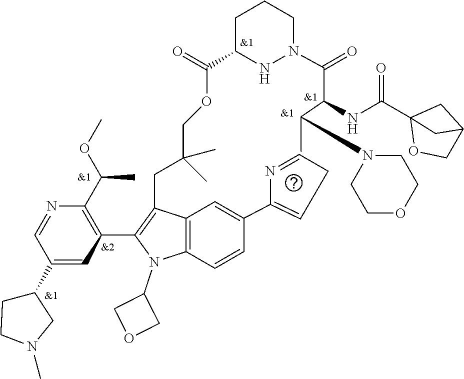 |
| A570 | 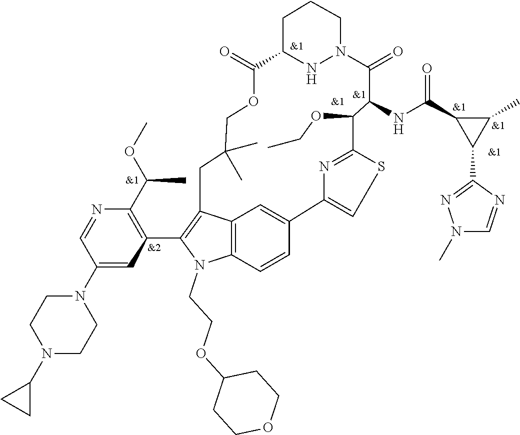 |
| A571 | 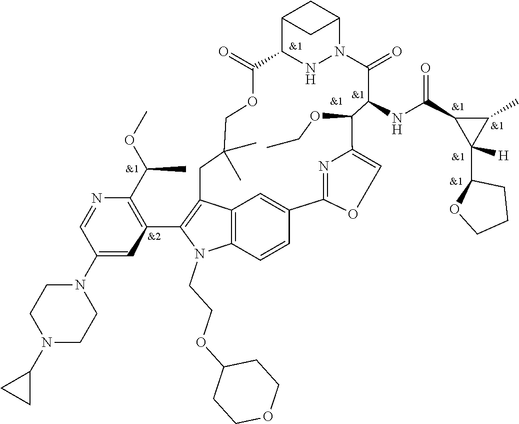 |
| A572 | 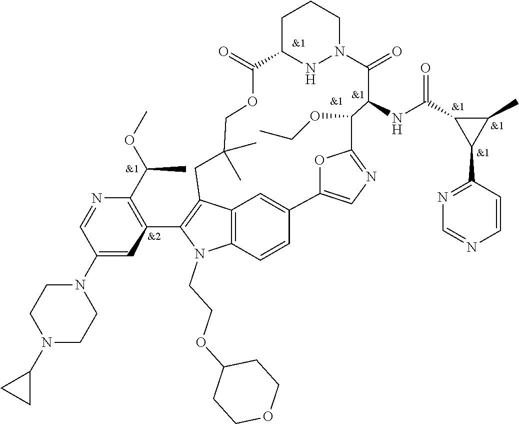 |
| A573 | 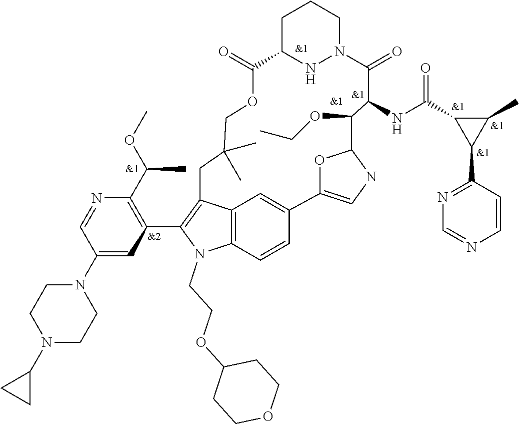 |
| A574 | 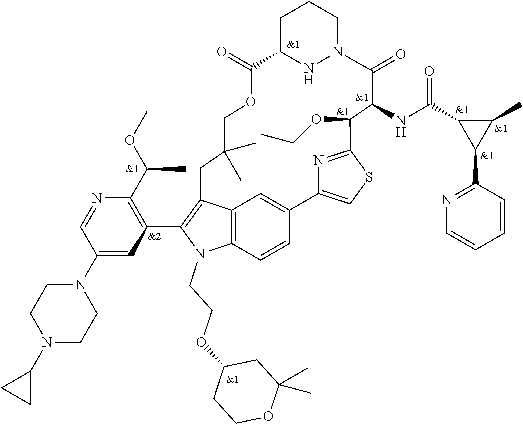 |
| A575 | 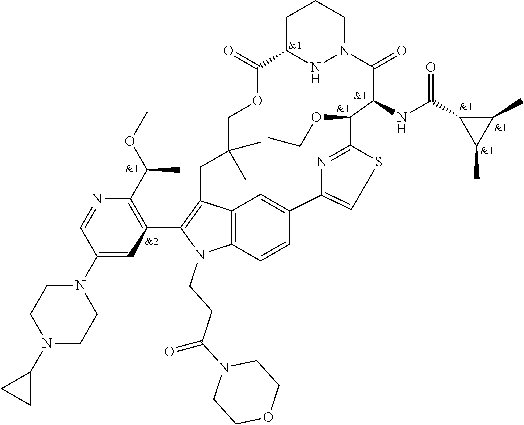 |
| A576 | 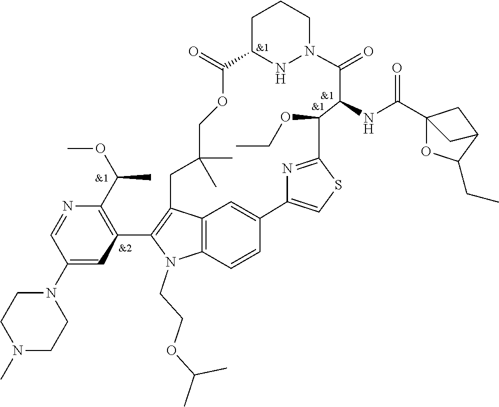 |
| A577 | 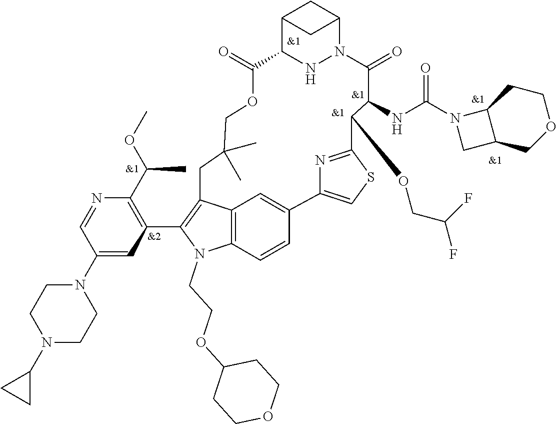 |
| A578 | 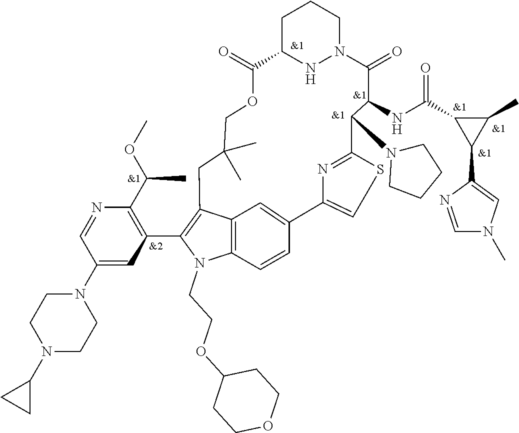 |
| A579 | 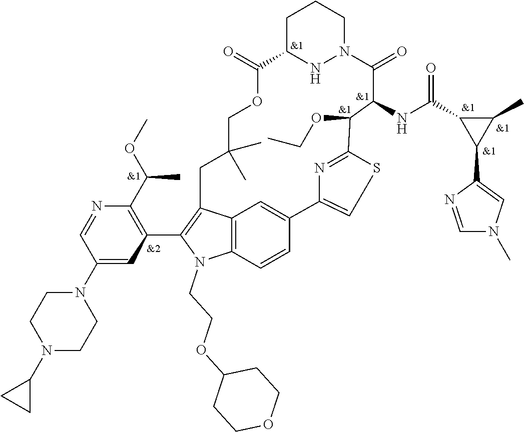 |
| A580 | 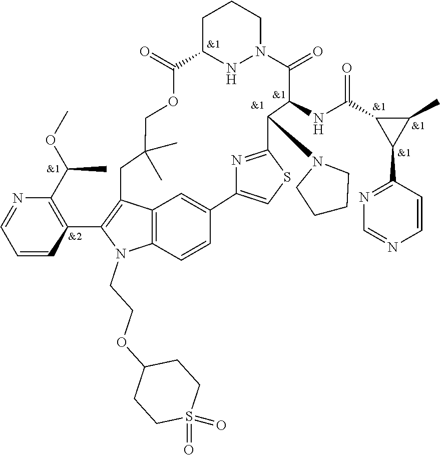 |
| A581 | 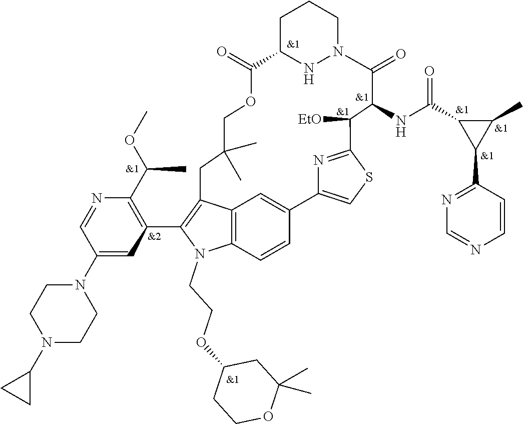 |
| A582 | 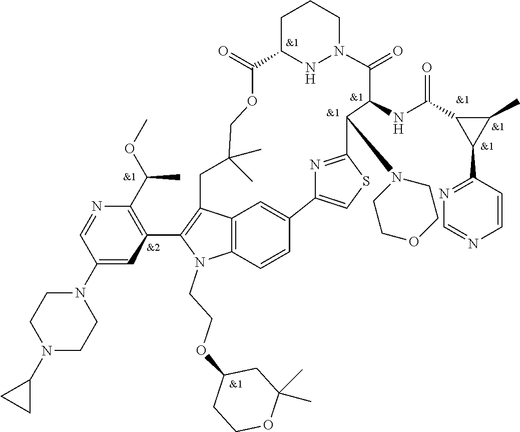 |
| A583 | 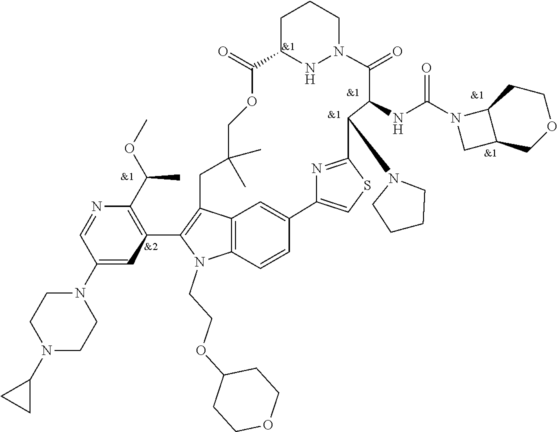 |
| A584 | 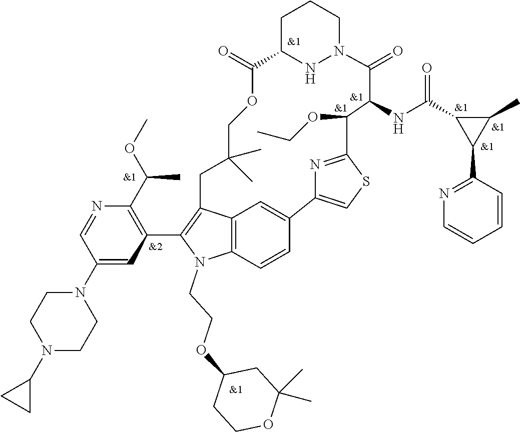 |
| A585 | 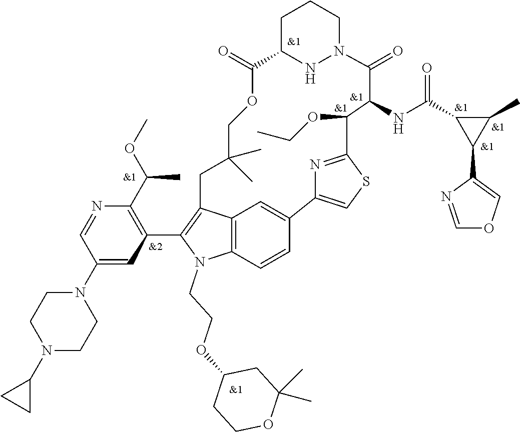 |
| A586 | 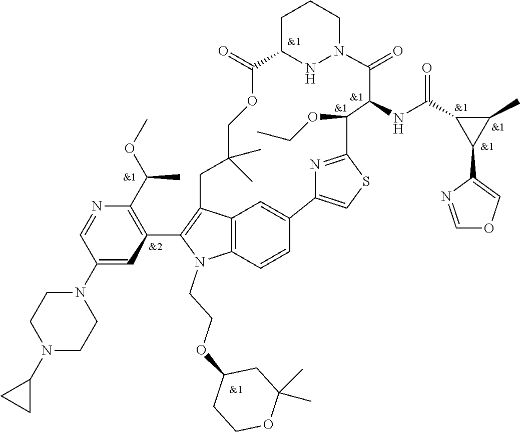 |
| A587 | 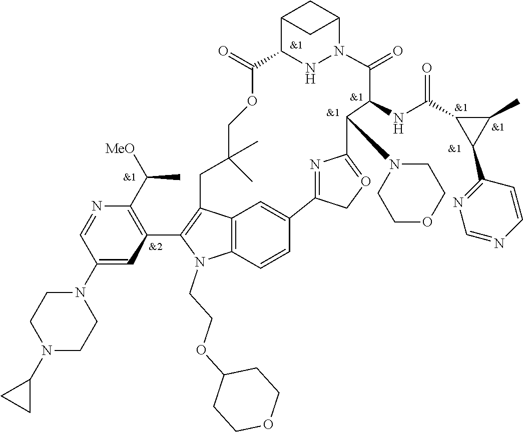 |
| A588 | 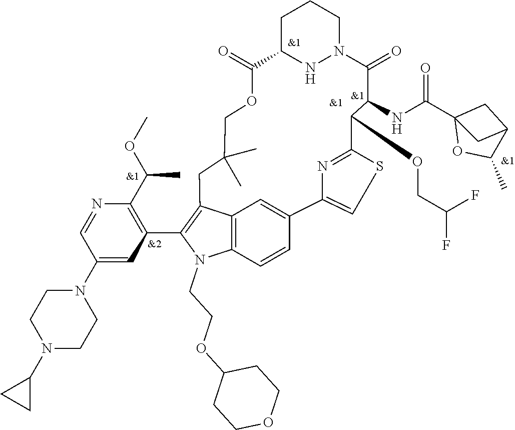 |
| A589 | 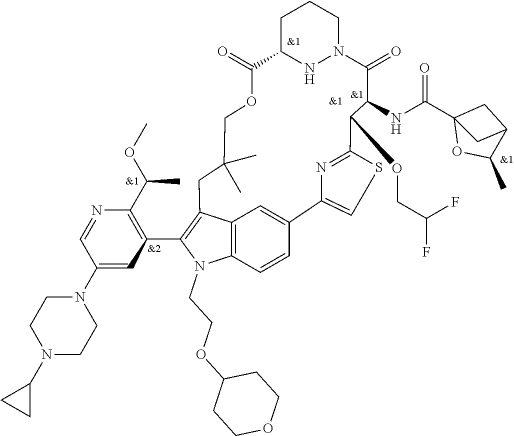 |
| A590 | 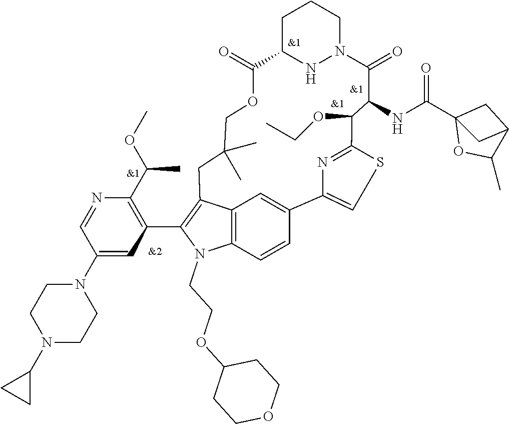 |
| A591 | 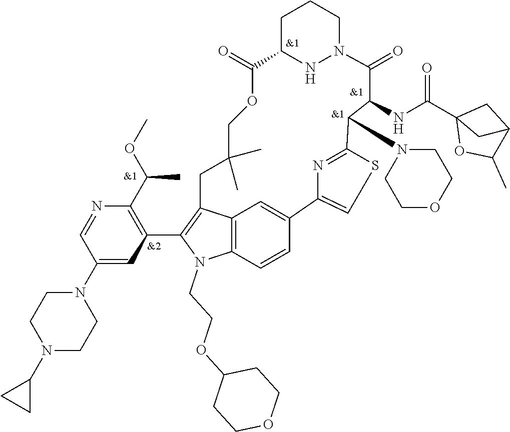 |
| A592 | 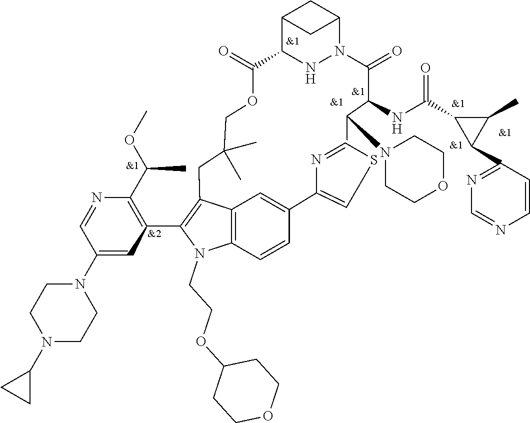 |
| A593 | 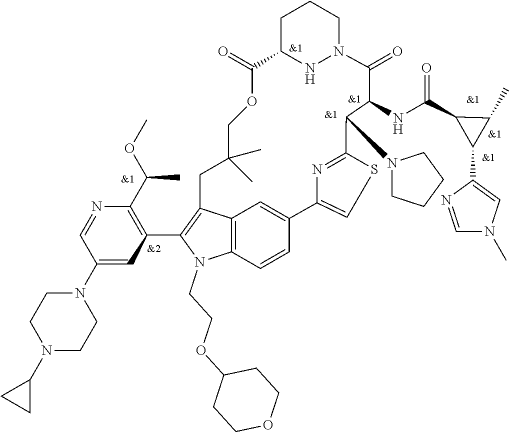 |
| A594 | 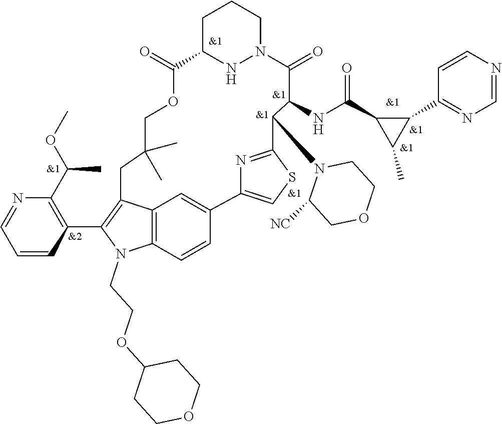 |
| A595 | 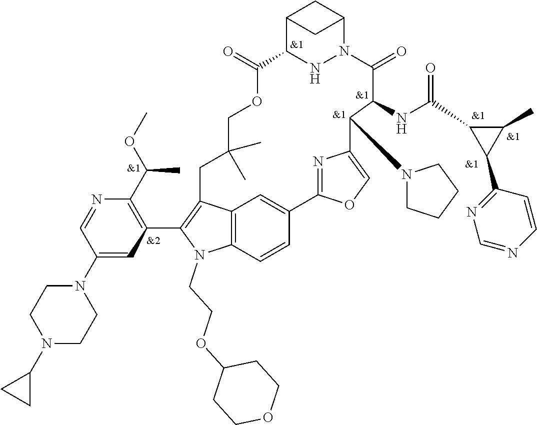 |
| A596 | 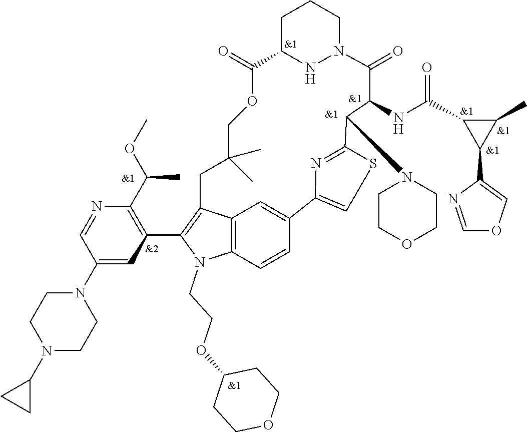 |
| A597 | 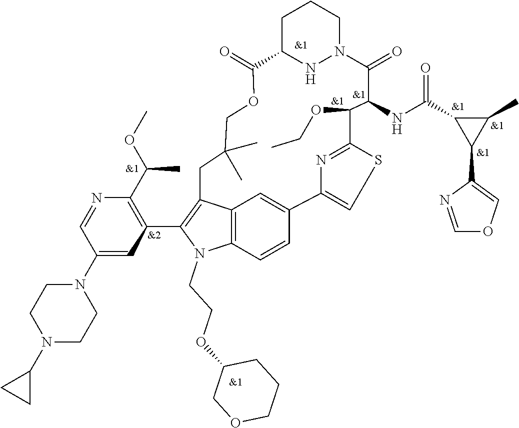 |
| A598 | 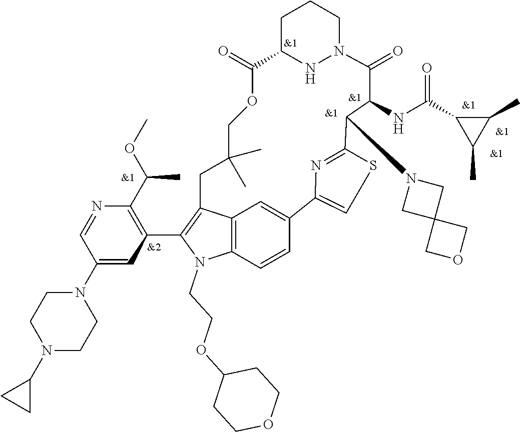 |
| A599 | 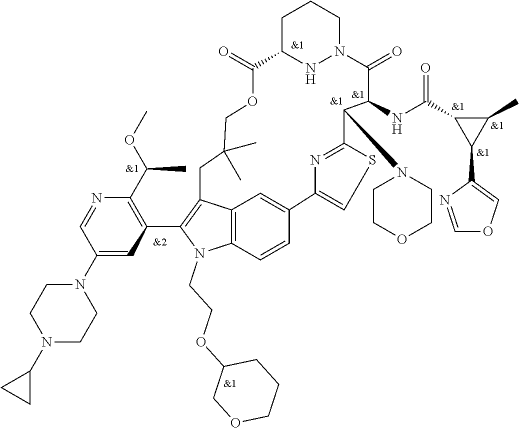 |
| A600 | 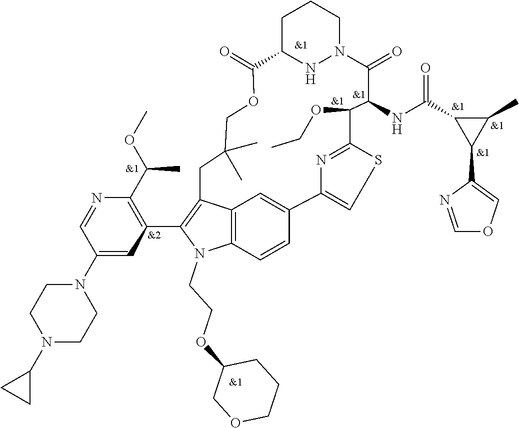 |
| A601 | 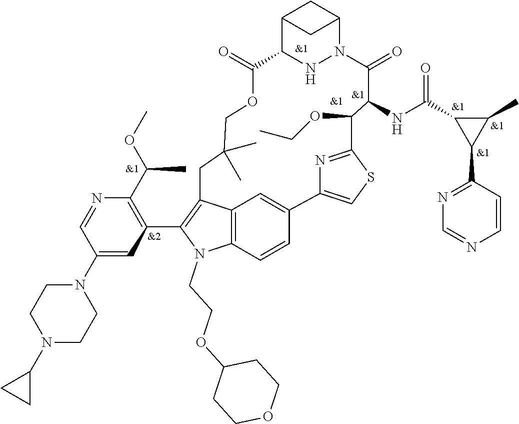 |
| A602 | 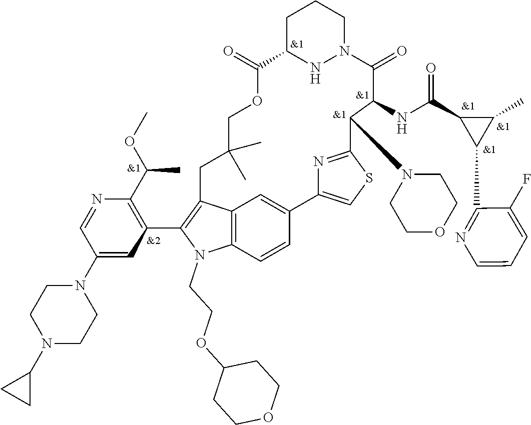 |
| A603 | 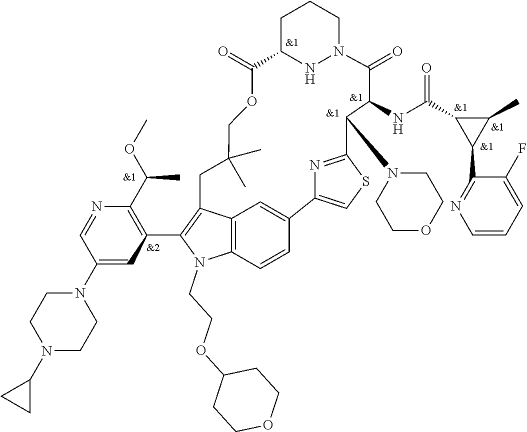 |
| A604 | 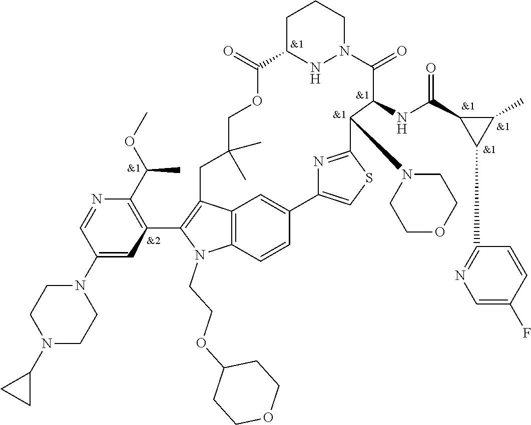 |
| A605 | 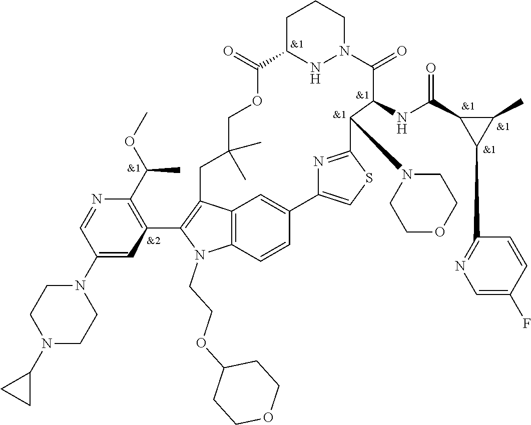 |
| A606 | 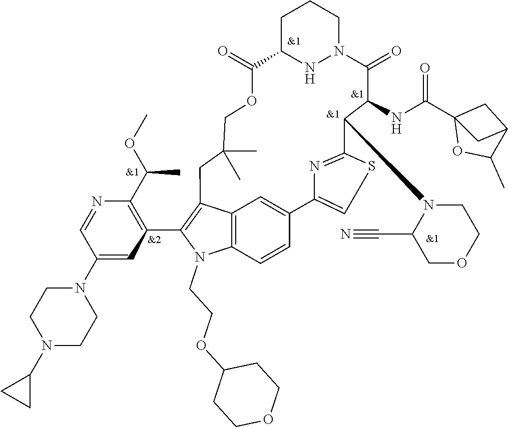 |
| A607 | 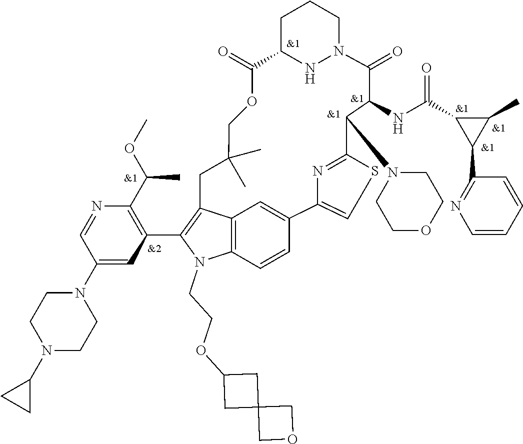 |
| A608 | 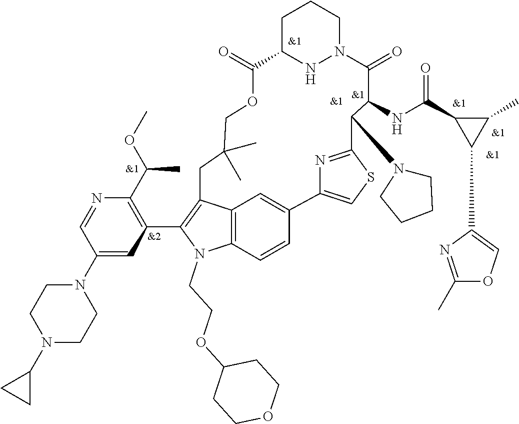 |
| A609 | 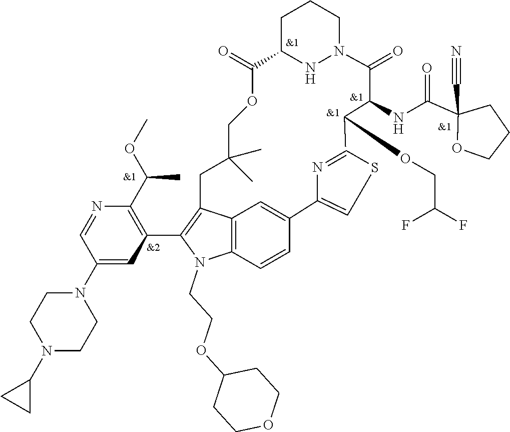 |
| A610 | 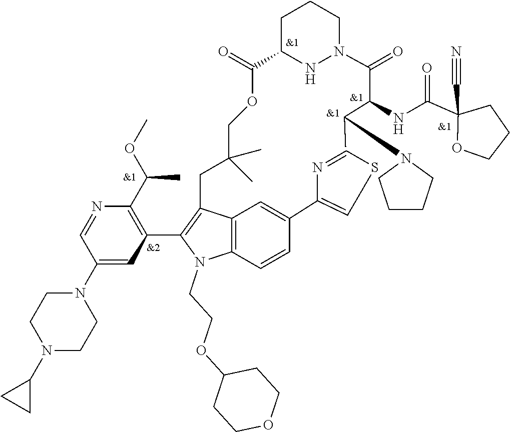 |
| A611 | 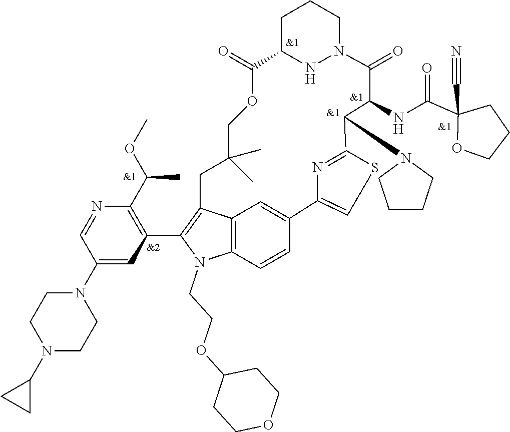 |
| A612 | 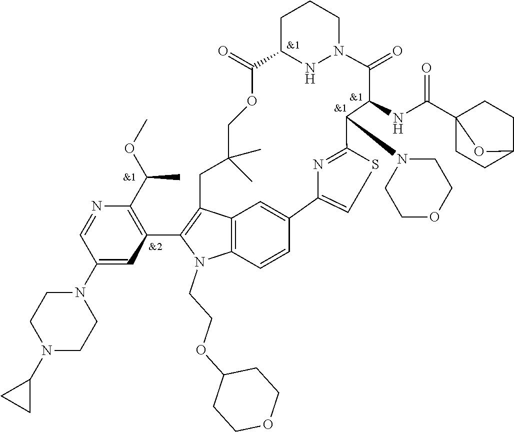 |
| A613 | 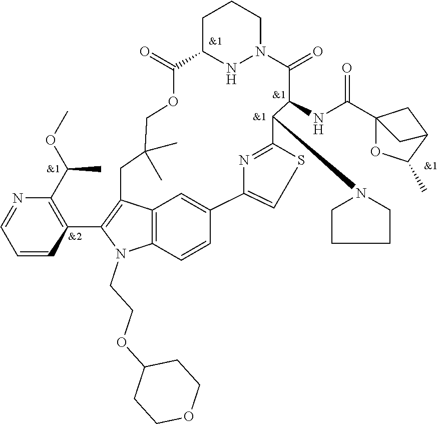 |
| A614 | 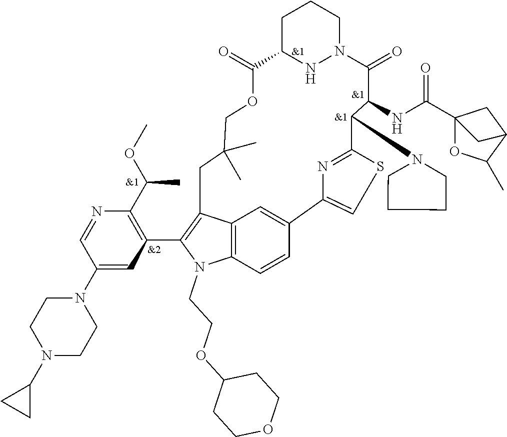 |
| A615 | 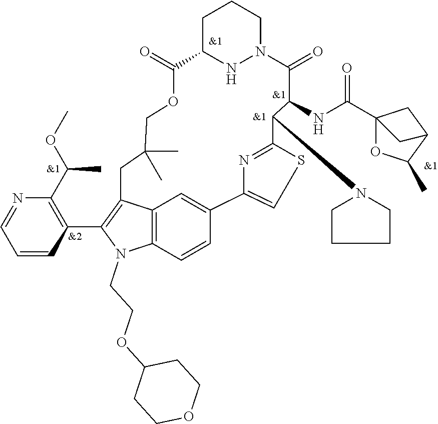 |
| A616 | 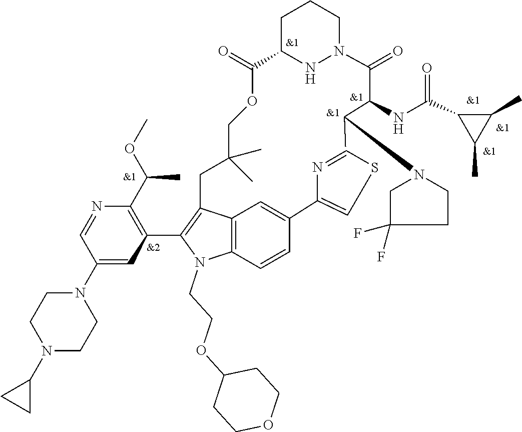 |
| A617 | 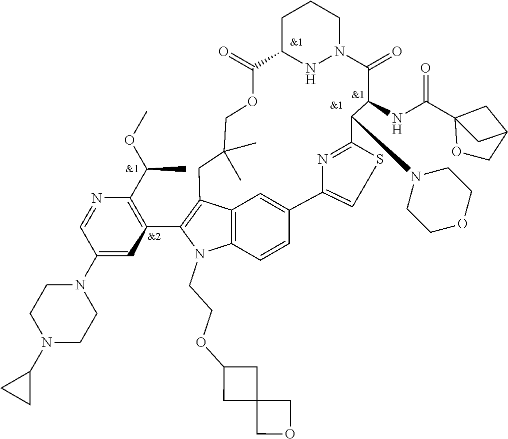 |
| A618 | 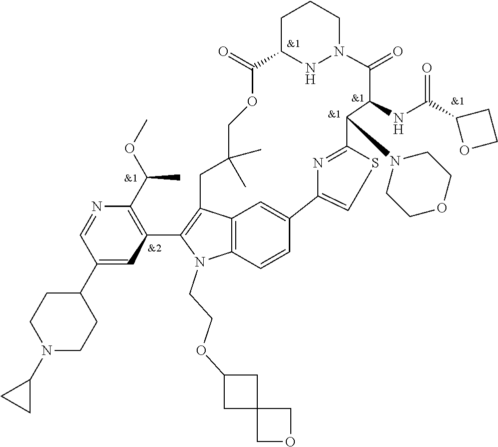 |
| A619 | 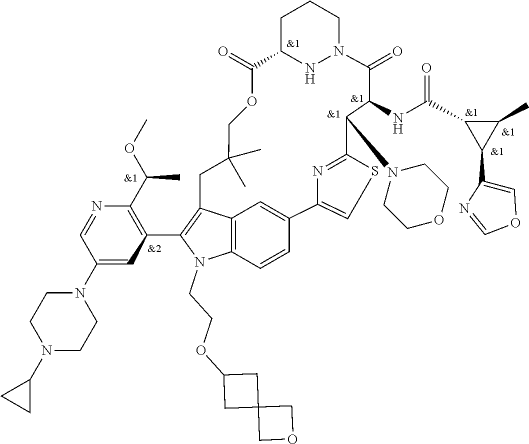 |
| A620 | 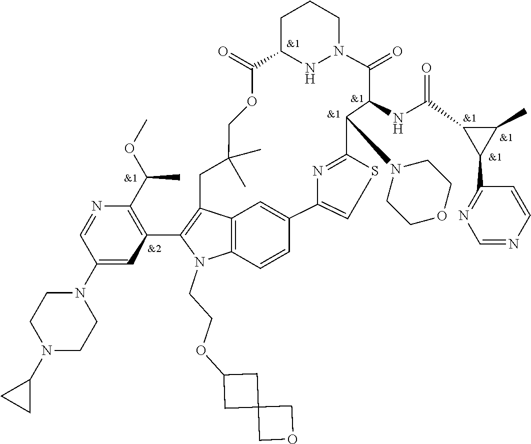 |
| A621 | 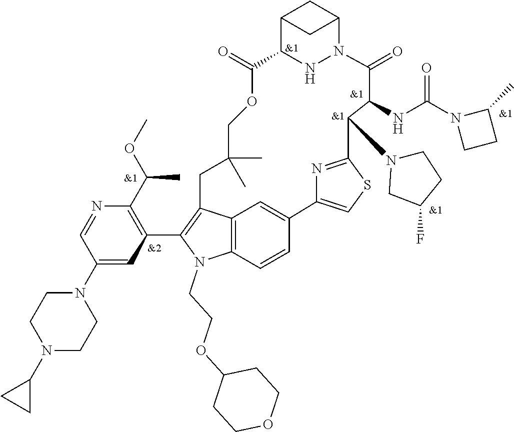 |
| A622 | 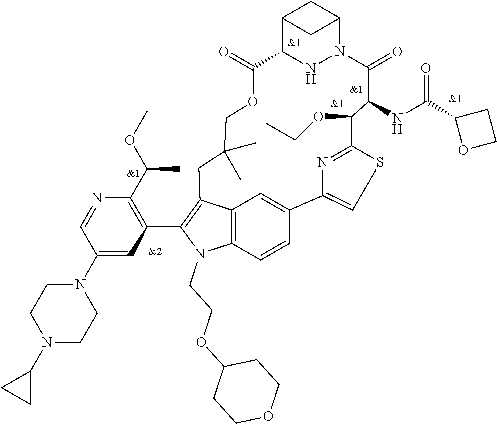 |
| A623 | 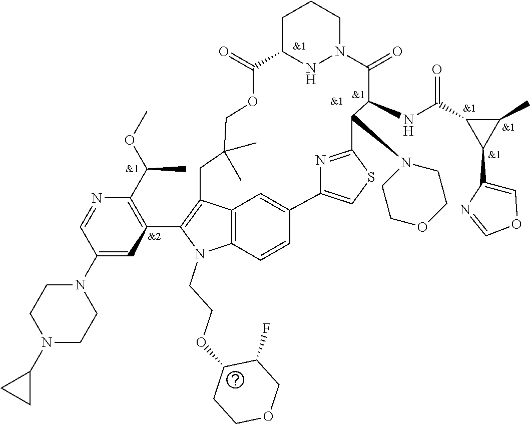 |
| A624 | 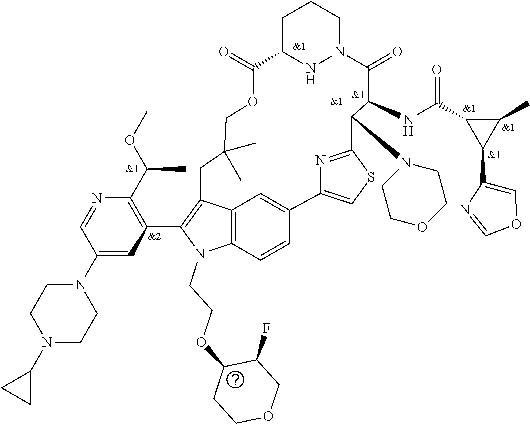 |
| A625 | 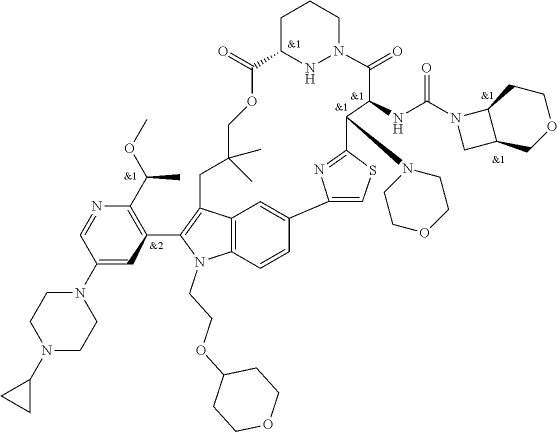 |
| A626 | 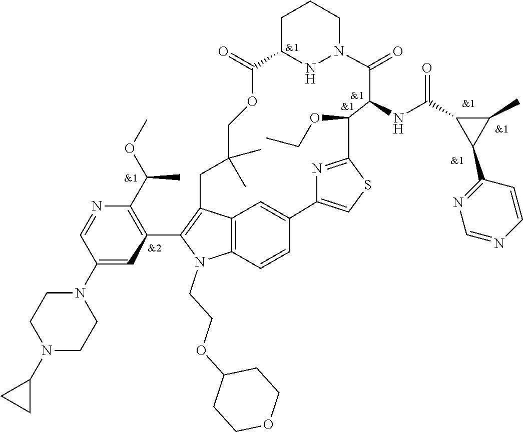 |
| A627 | 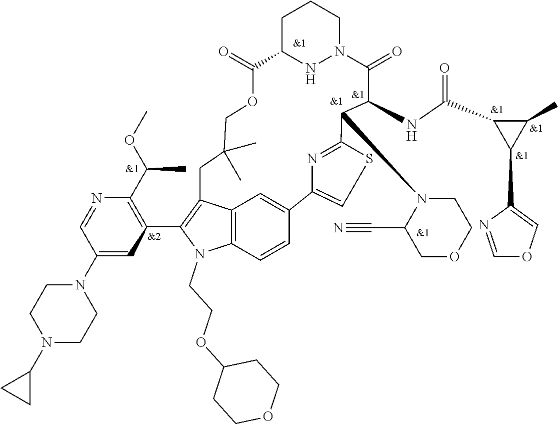 |
| A628 | 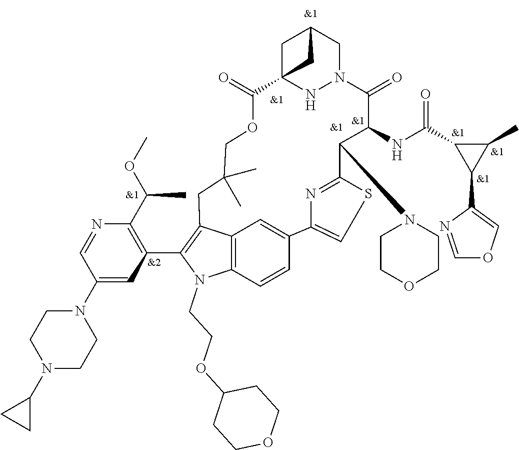 |
| A629 | 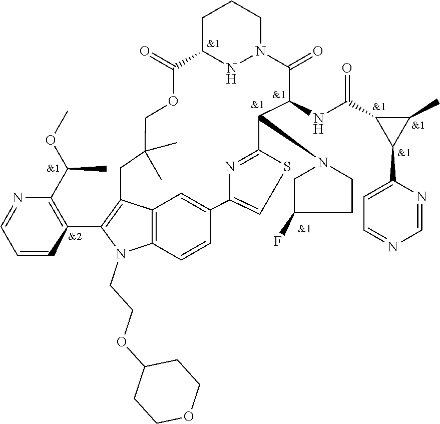 |
| A630 | 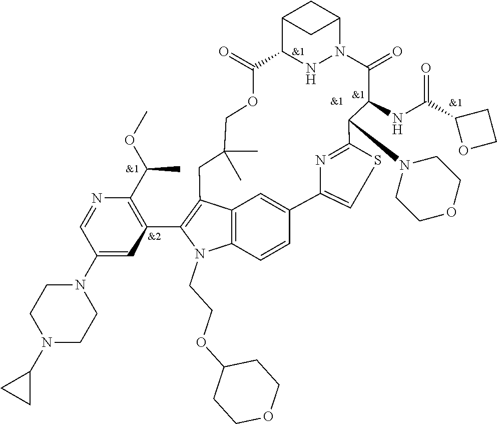 |
| A631 | 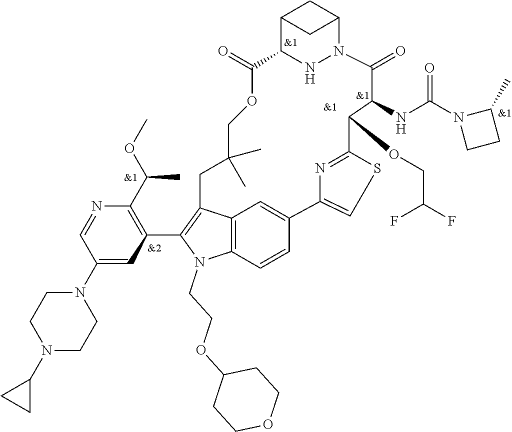 |
| A632 | 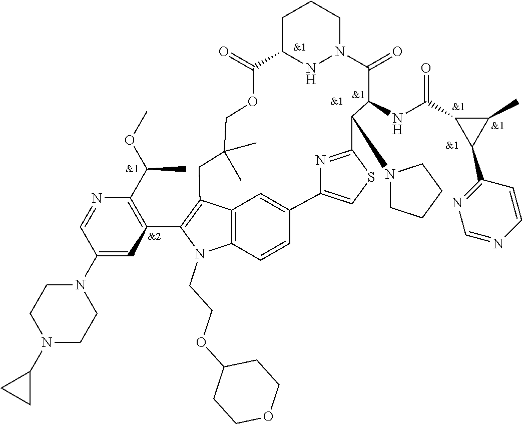 |
| A633 | 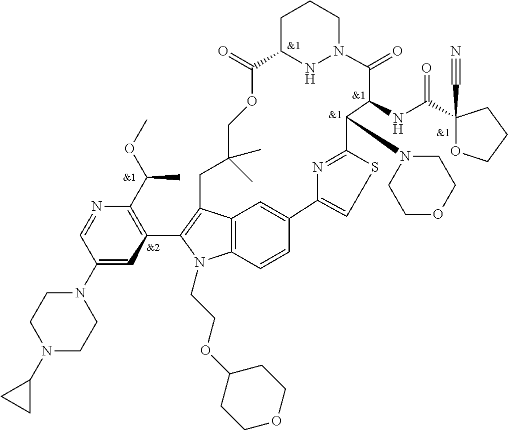 |
| A634 | 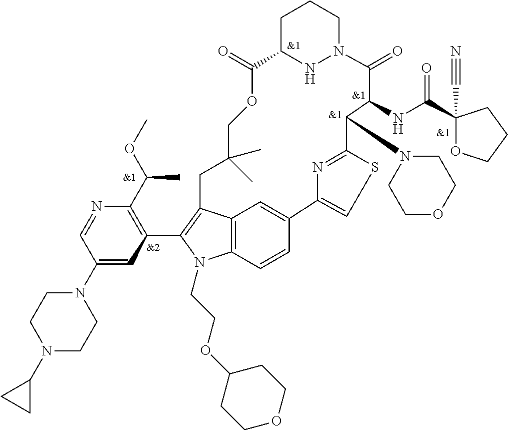 |
| A635 | 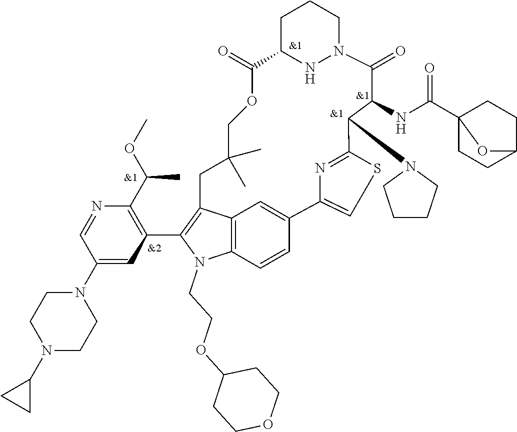 |
| A636 | 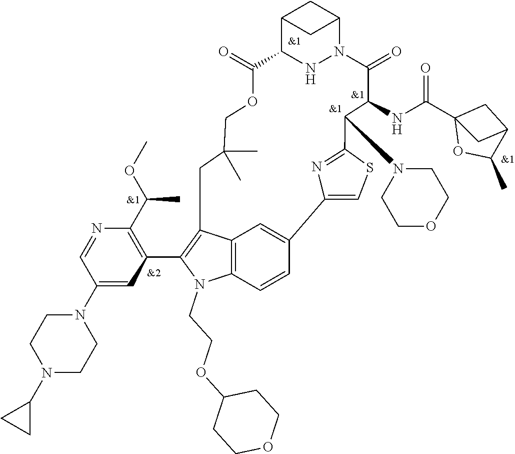 |
| A637 | 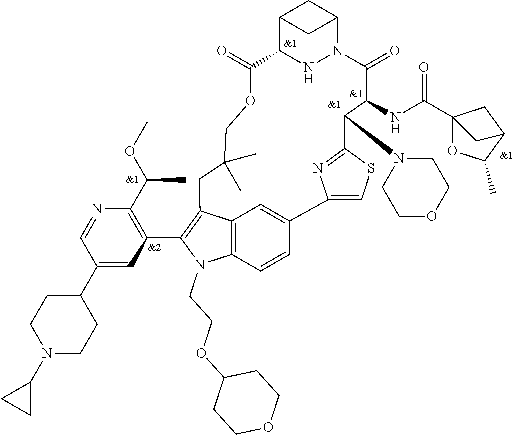 |
| A638 | 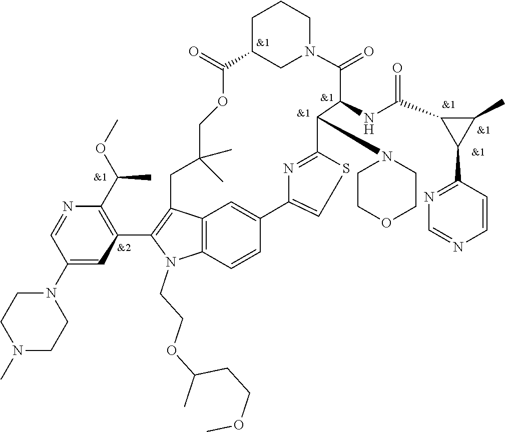 |
| A639 | 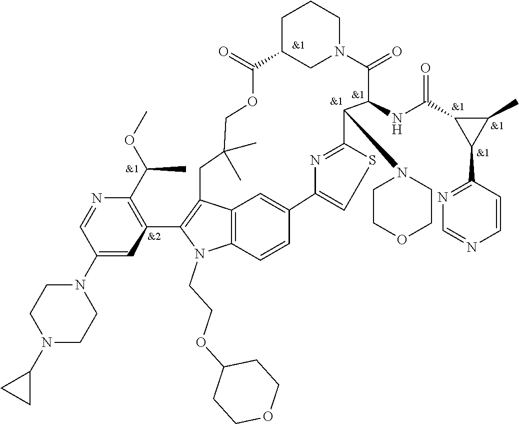 |
| A640 | 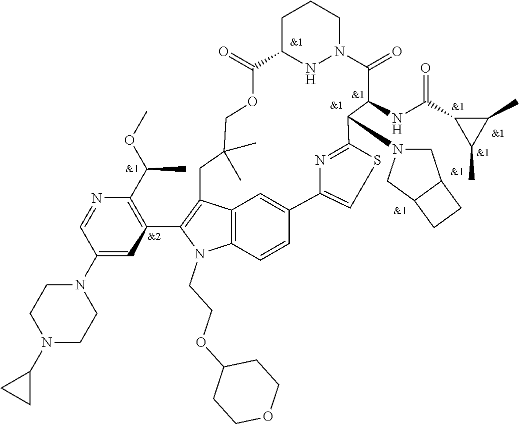 |
| A641 | 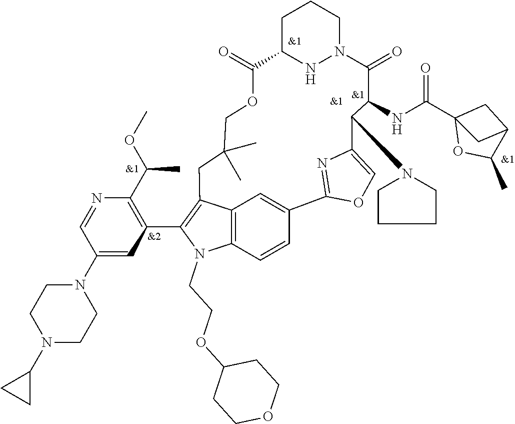 |
| A642 | 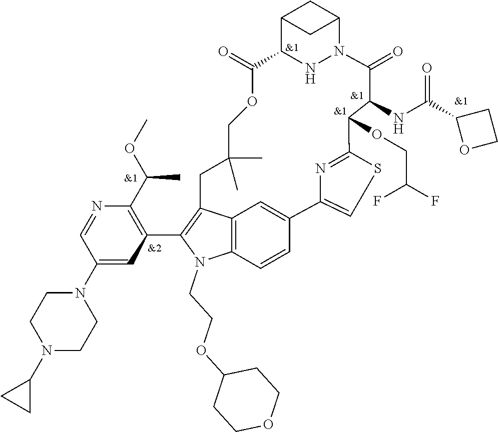 |
| A643 | 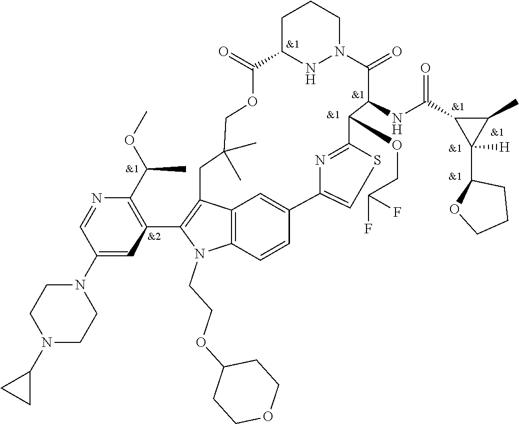 |
| A644 | 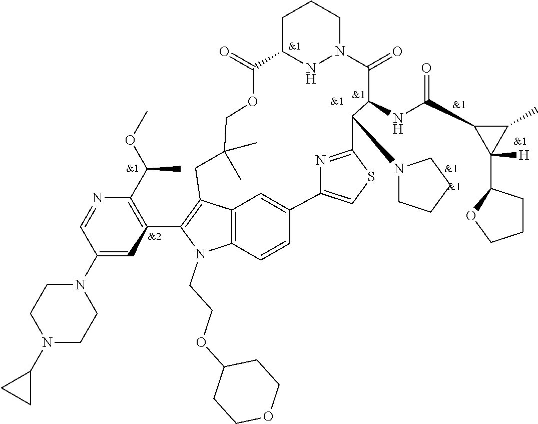 |
| A645 | 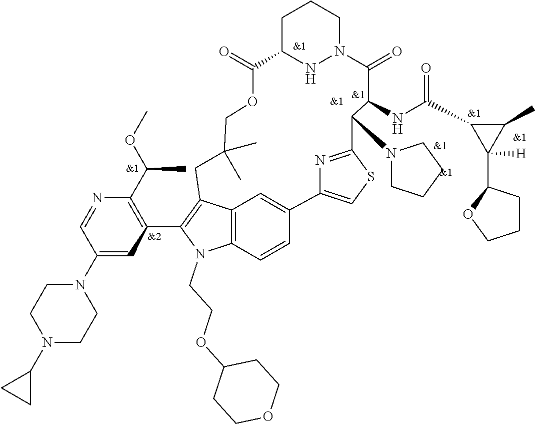 |
| A646 | 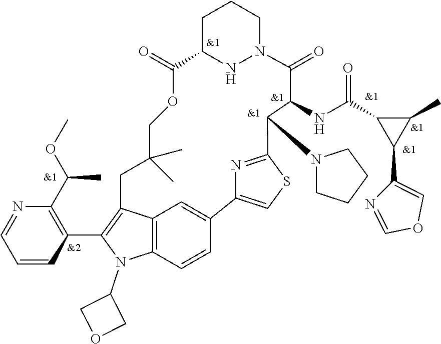 |
| A647 | 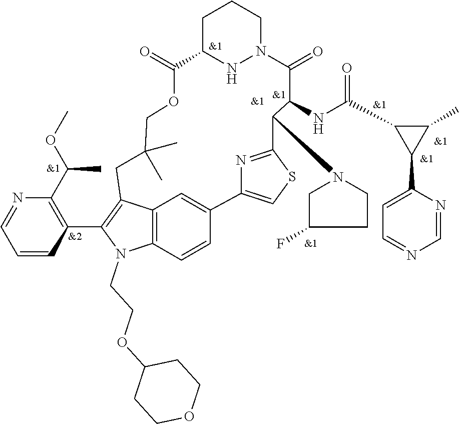 |
| A648 | 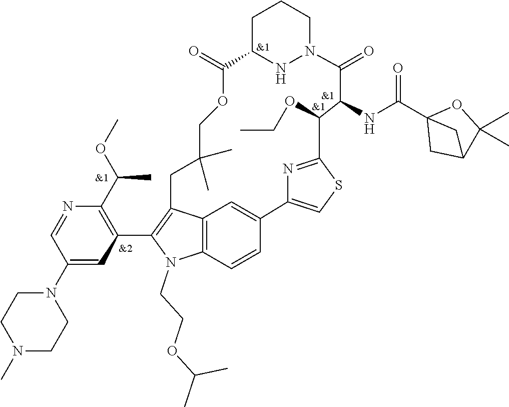 |
| A649 | 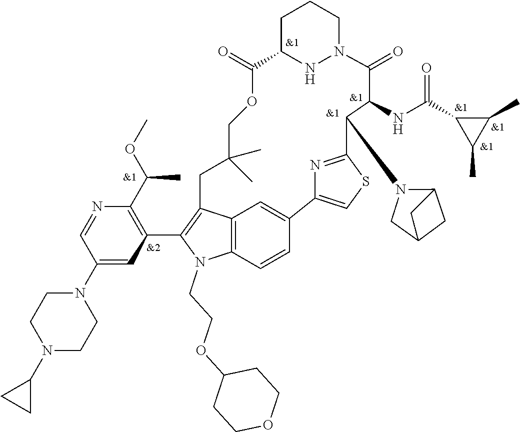 |
| A650 | 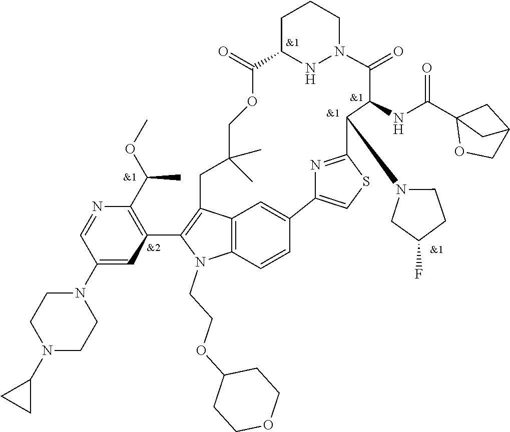 |
| A651 | 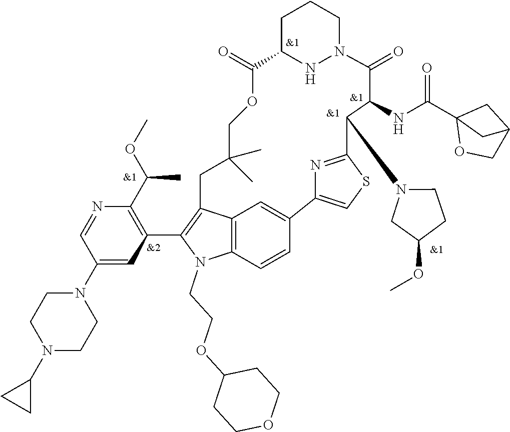 |
| A652 | 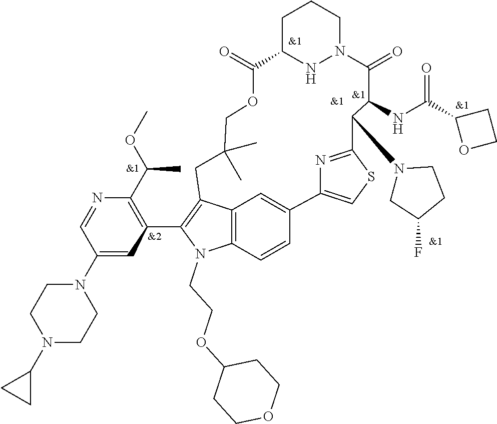 |
| A653 | 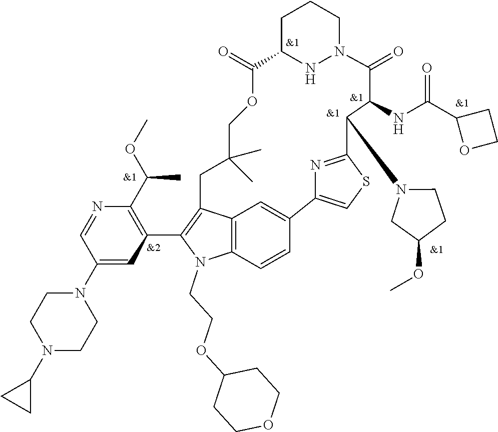 |
| A654 | 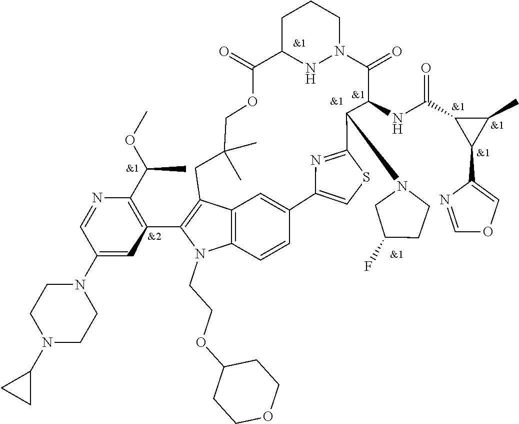 |
| A655 | 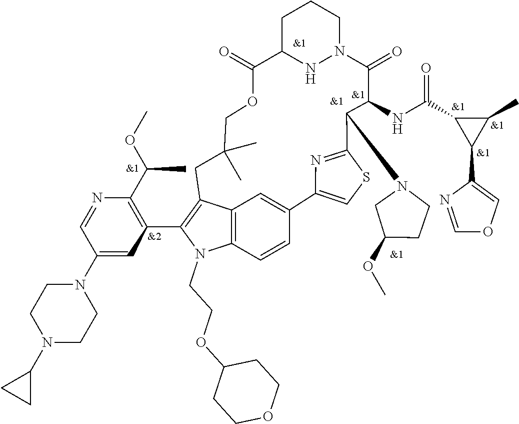 |
| A656 | 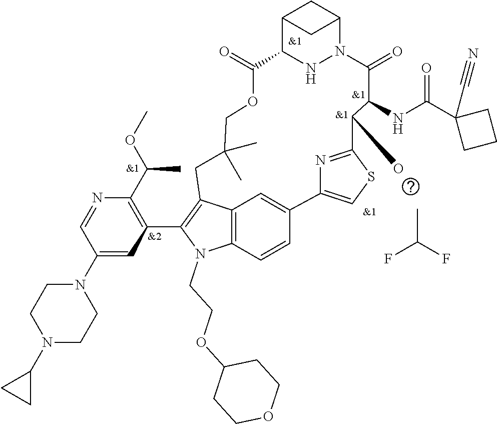 |
| A657 | 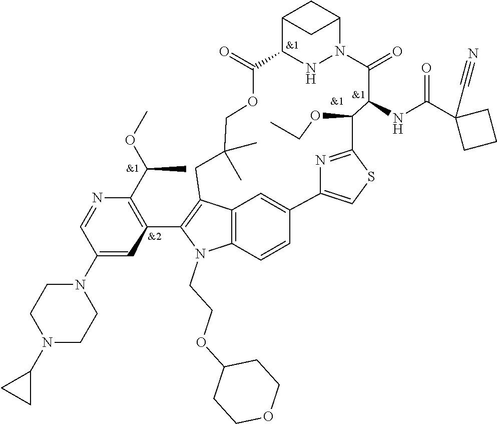 |
| A658 | 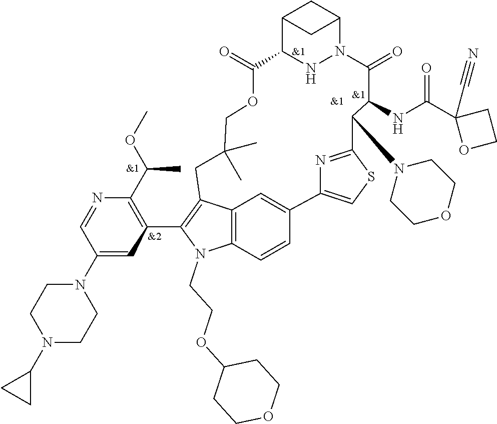 |
| A659 | 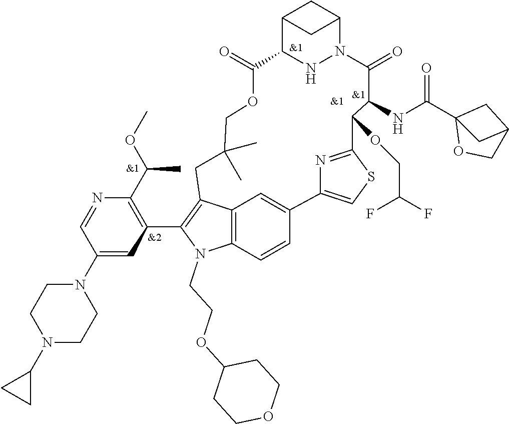 |
| A660 | 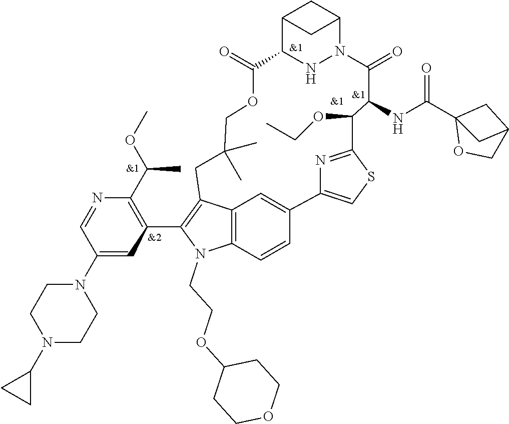 |
| A661 | 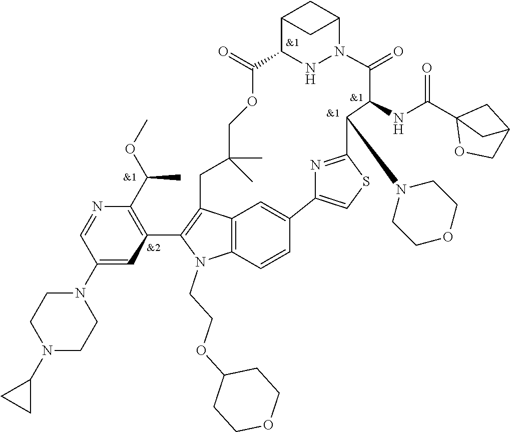 |
| A662 | 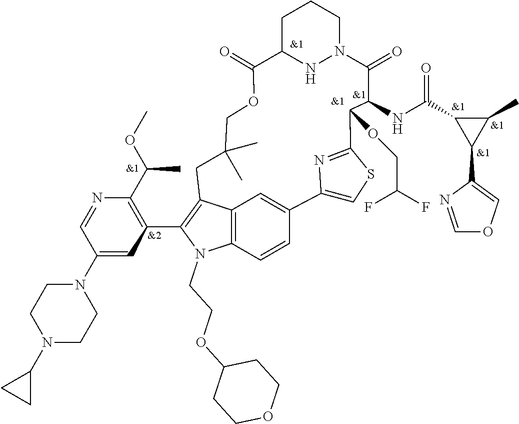 |
| A663 | 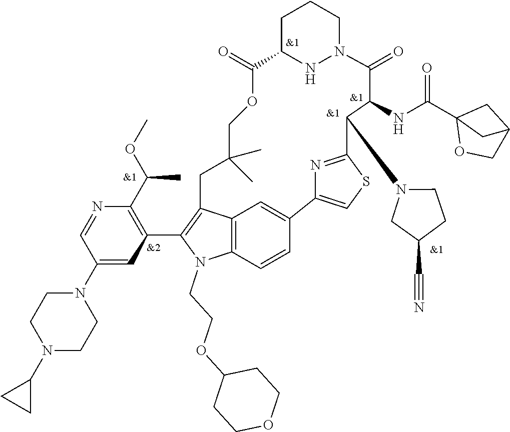 |
| A664 | 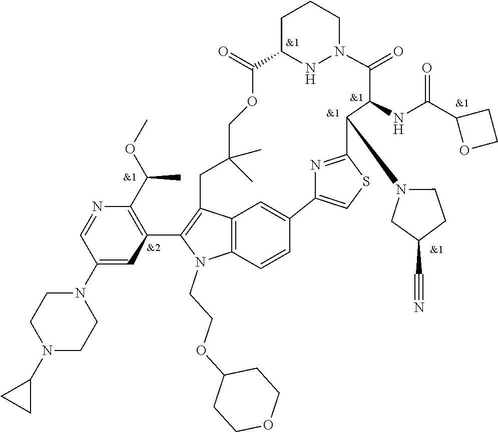 |
| A665 | 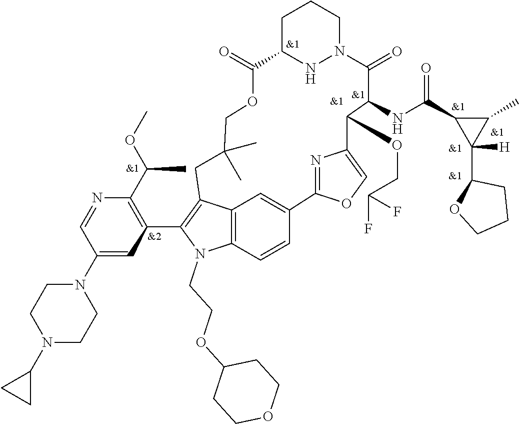 |
| A666 | 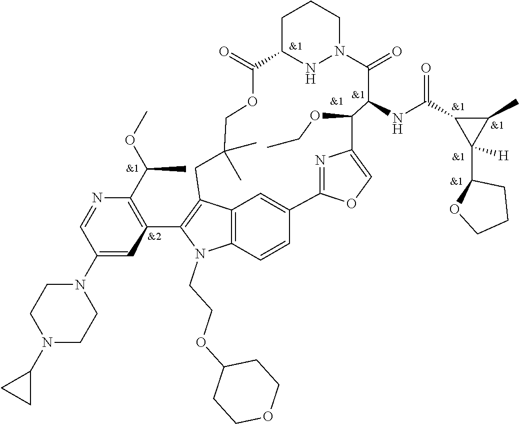 |
| A667 | 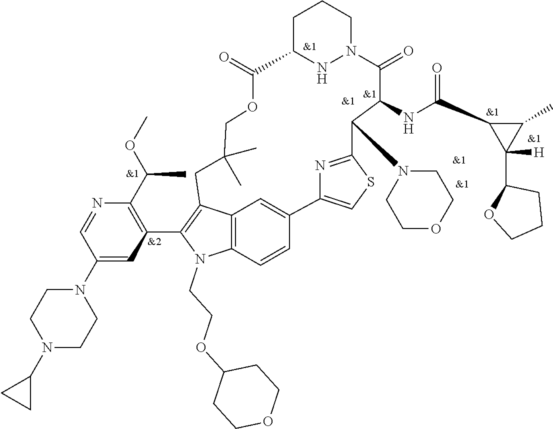 |
| A668 | 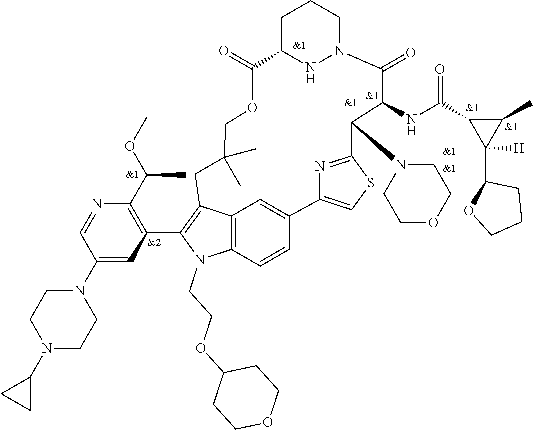 |
| A669 | 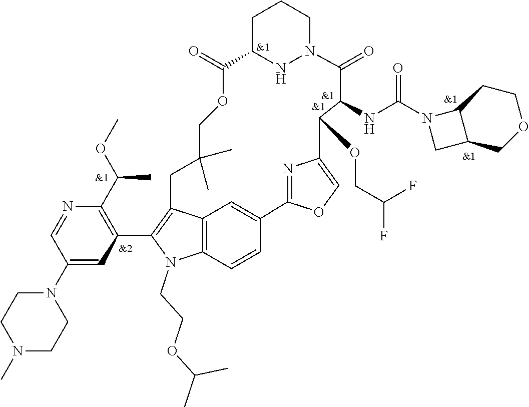 |
| A670 | 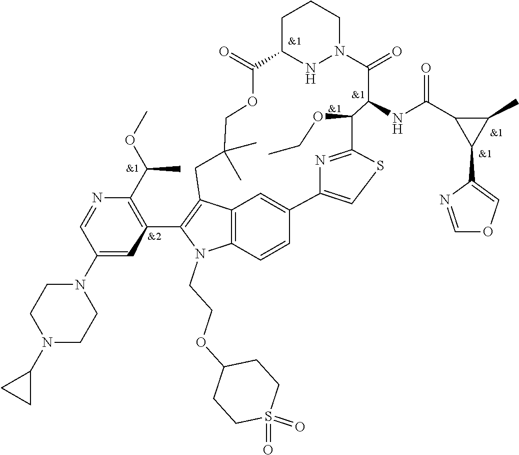 |
| A671 | 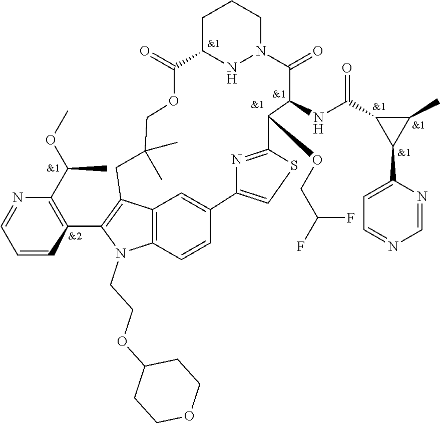 |
| A672 | 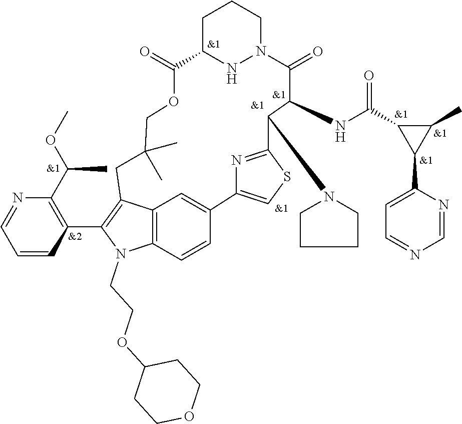 |
| A673 | 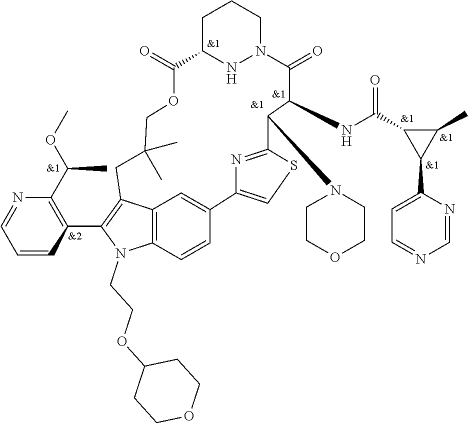 |
| A674 | 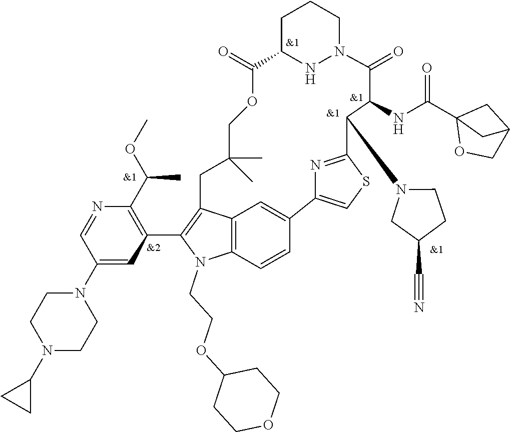 |
| A675 | 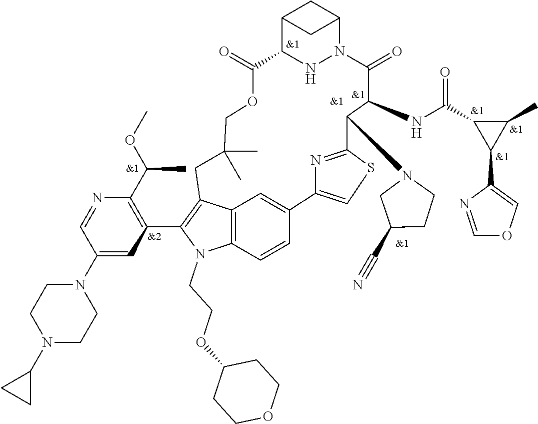 |
| A676 | 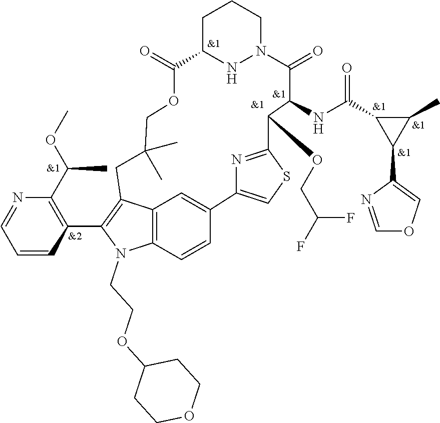 |
| A677 | 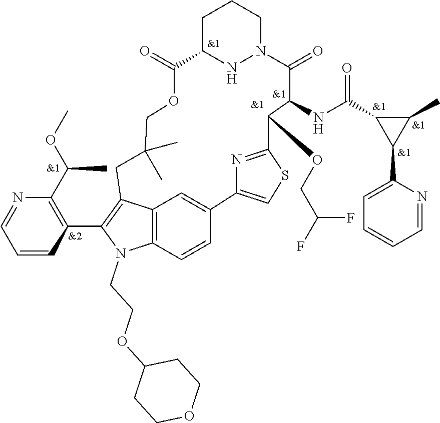 |
| A678 | 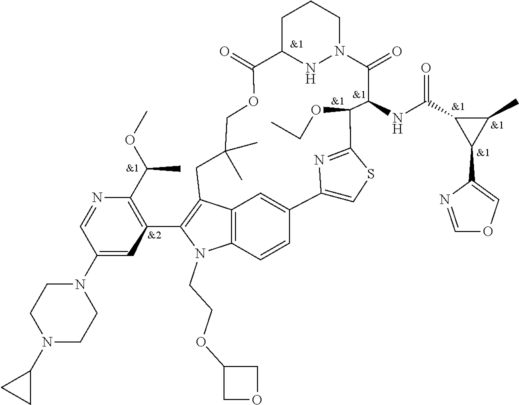 |
| A679 | 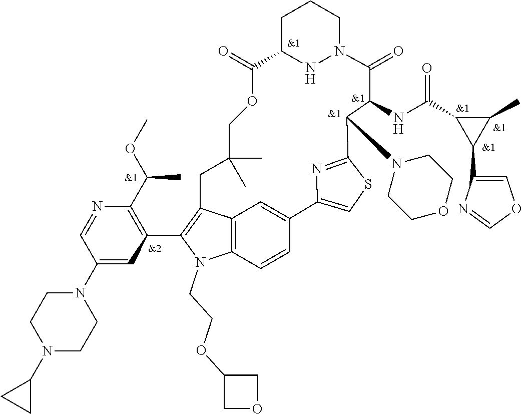 |
| A680 | 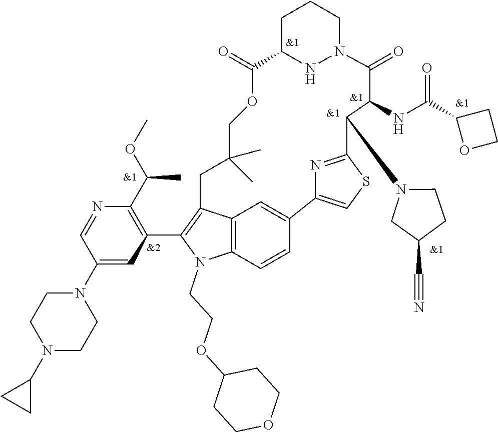 |
| A681 | 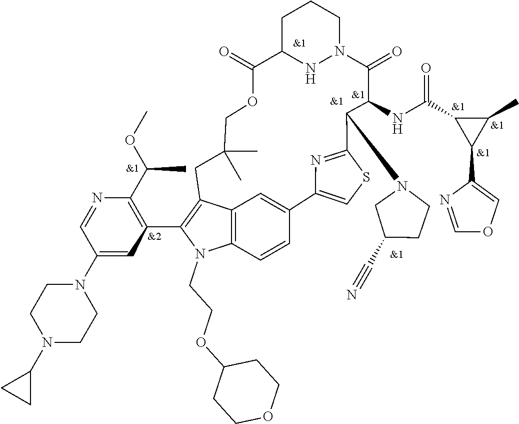 |
| A682 | 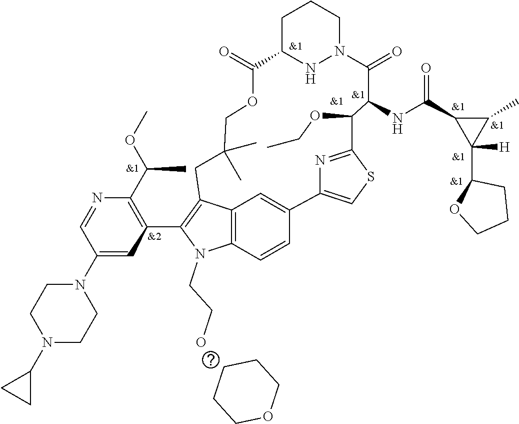 |
| A683 | 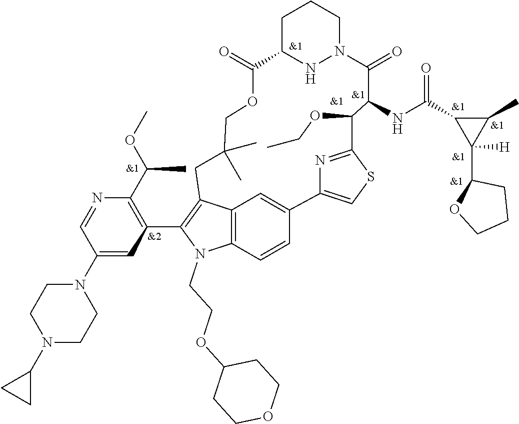 |
| A684 | 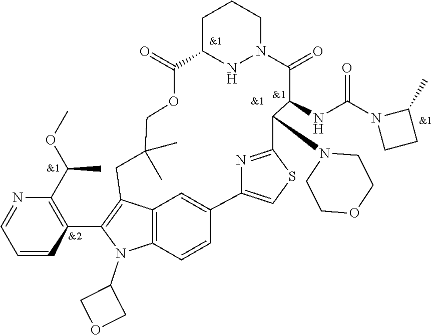 |
| A685 | 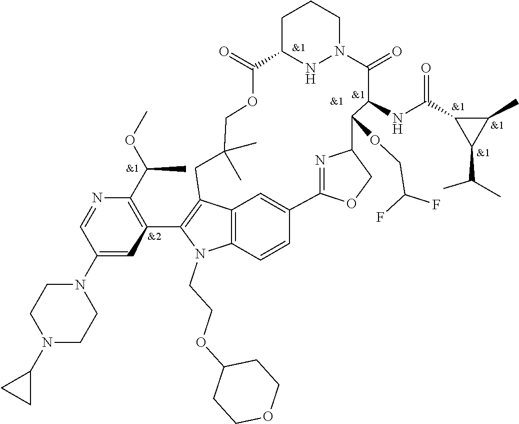 |
| A686 | 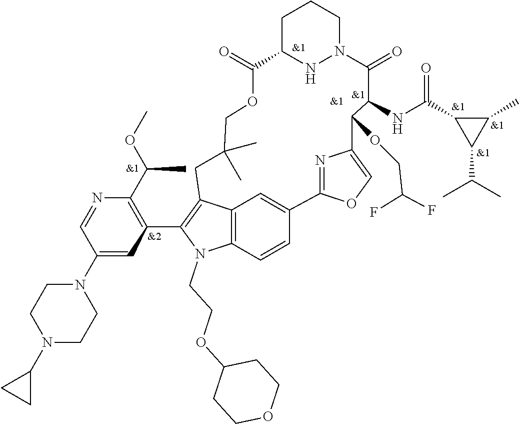 |
| A687 | 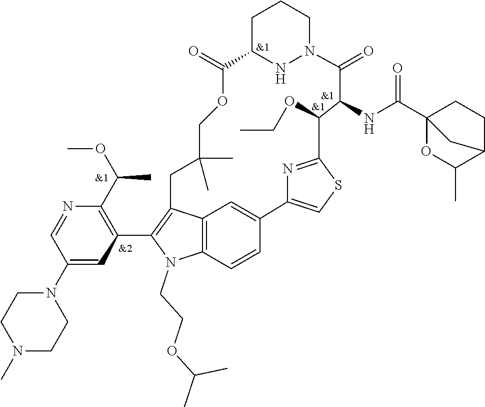 |
| A688 | 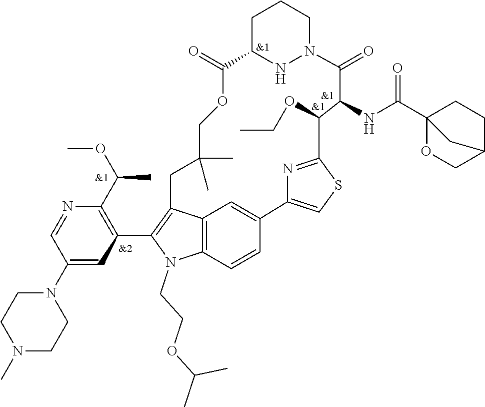 |
| A689 | 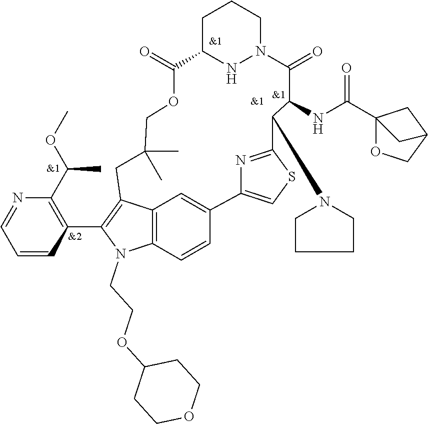 |
| A690 | 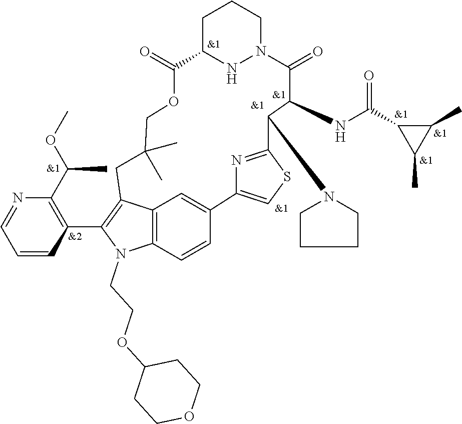 |
| A691 | 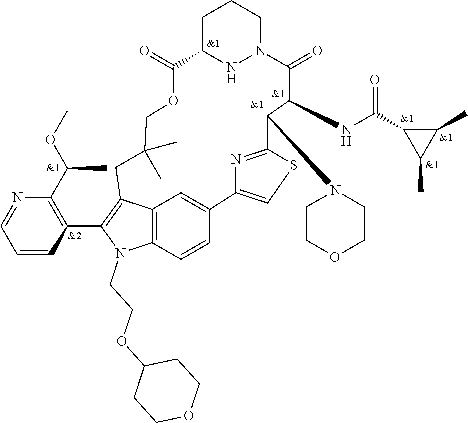 |
| A692 | 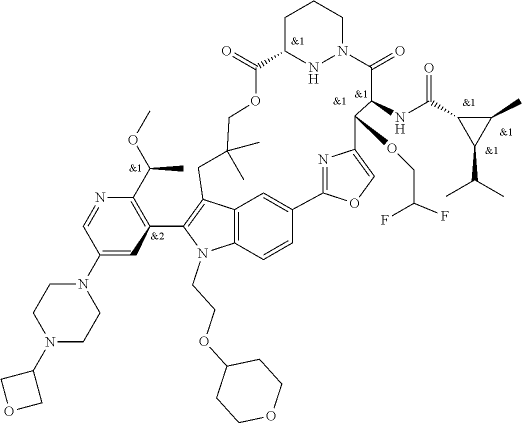 |
| A693 | 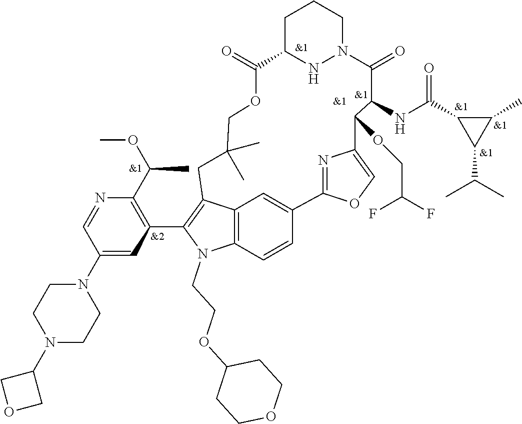 |
| A694 | 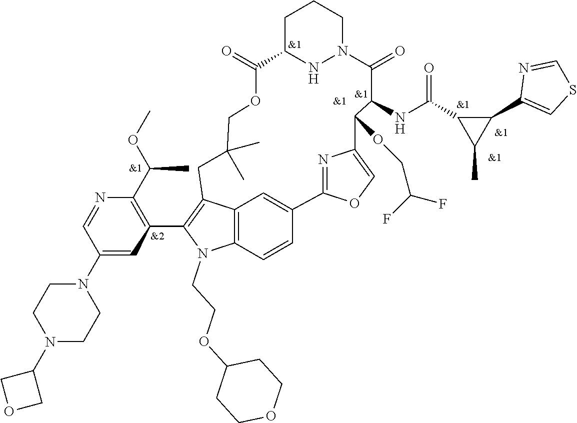 |
| A695 | 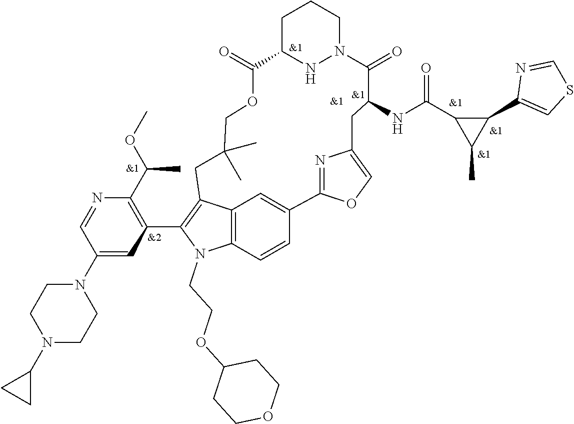 |
| A696 | 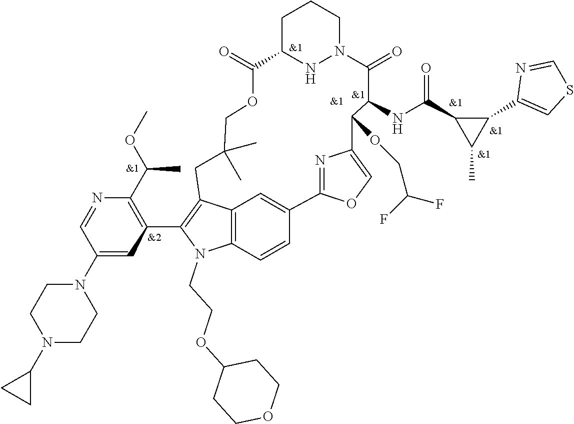 |
| A697 | 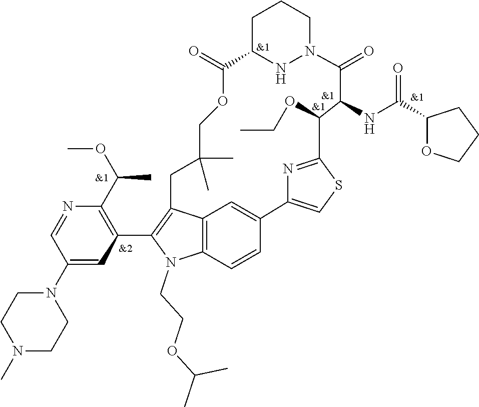 |
| A698 | 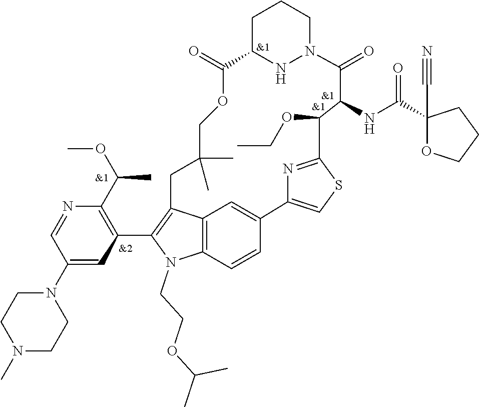 |
| A699 | 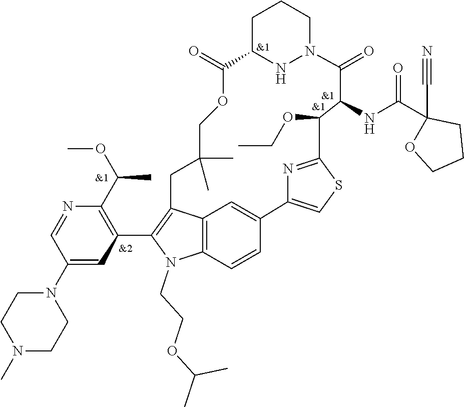 |
| A700 | 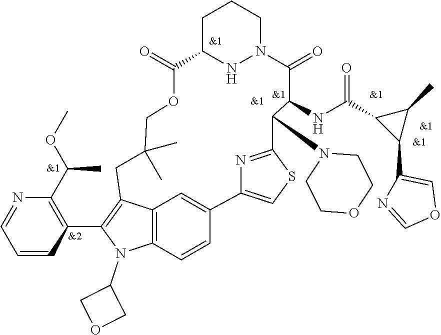 |
| A701 | 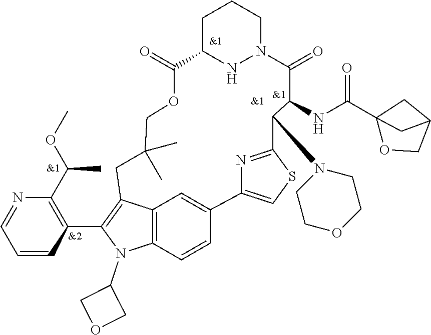 |
| A702 | 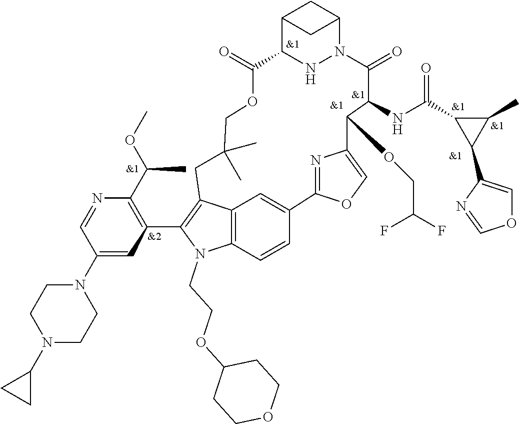 |
| A703 | 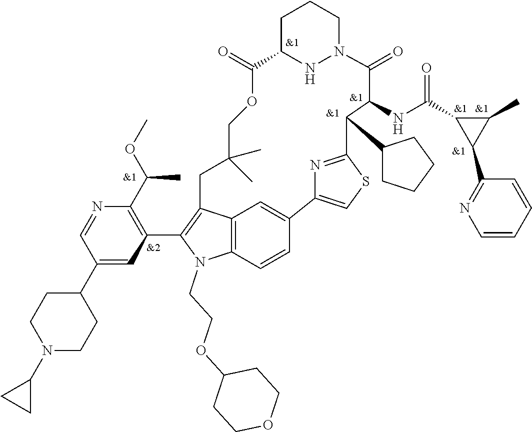 |
| A704 | 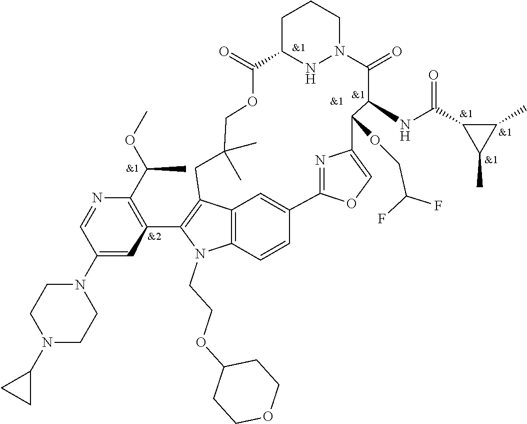 |
| A705 | 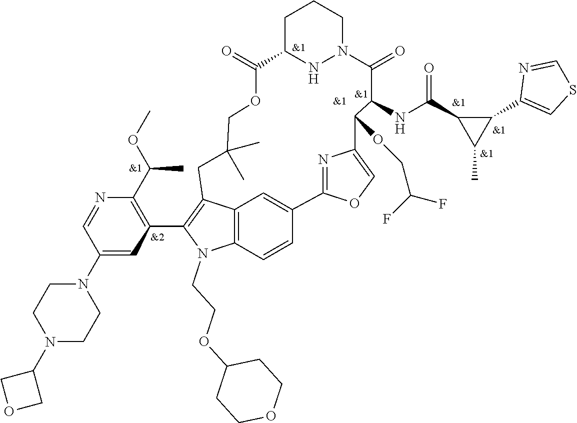 |
| A706 | 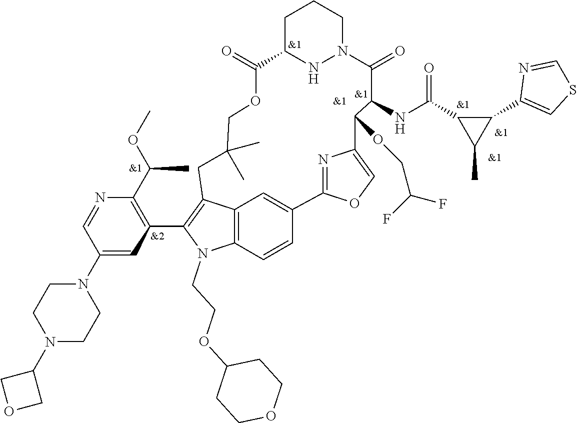 |
| A707 | 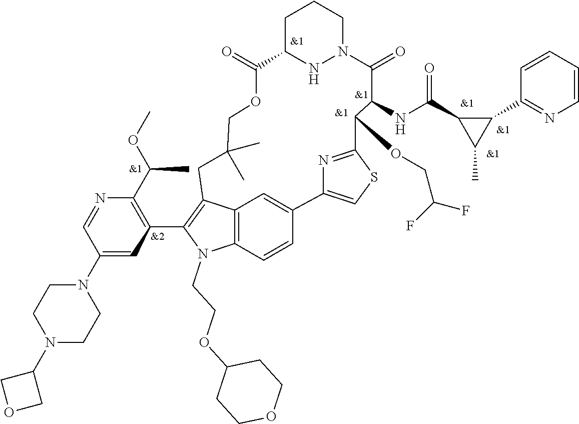 |
| A708 | 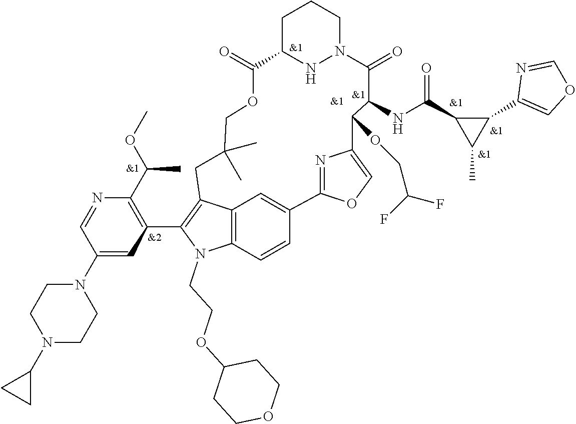 |
| A709 | 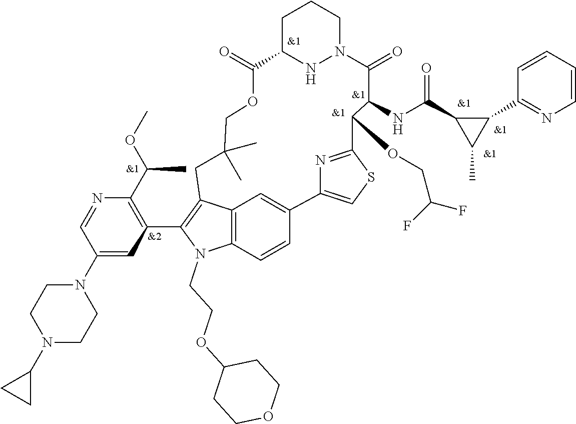 |
| A710 | 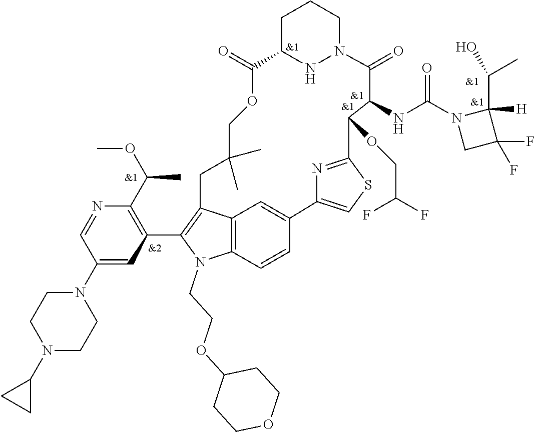 |
| A711 | 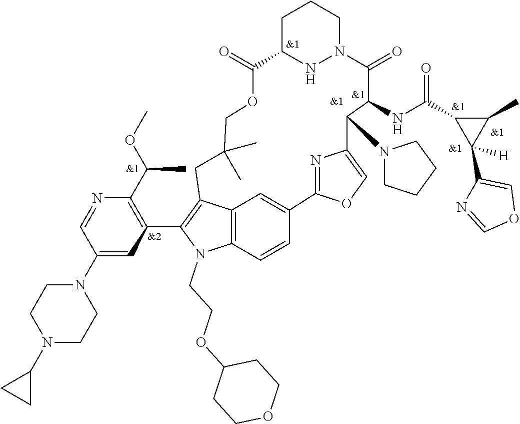 |
| A712 | 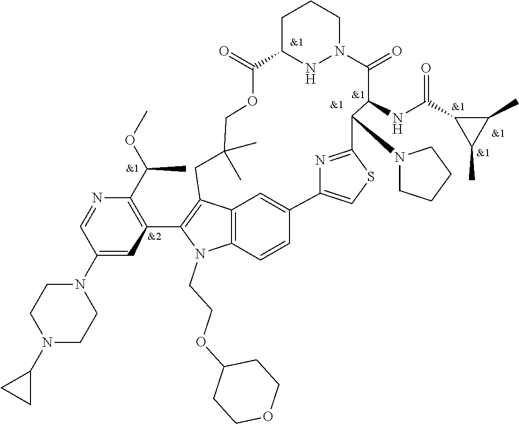 |
| A713 | 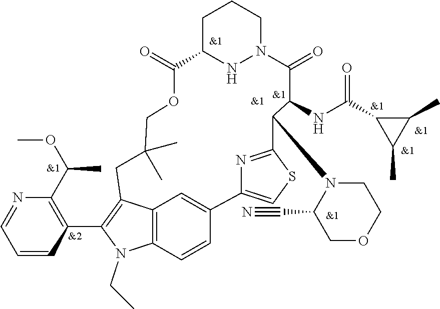 |
| A714 | 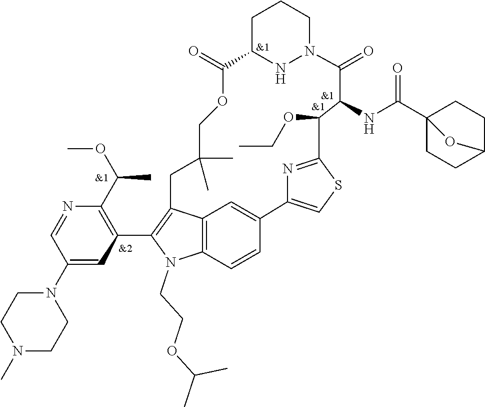 |
| A715 | 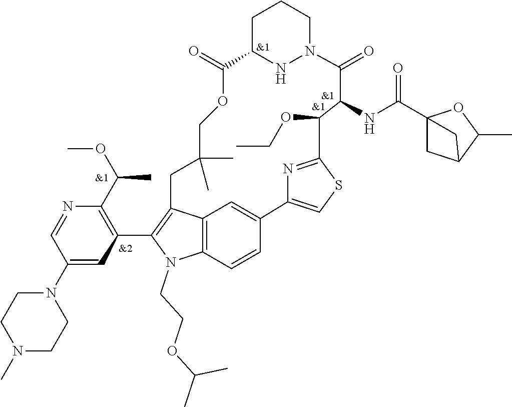 |
| A716 | 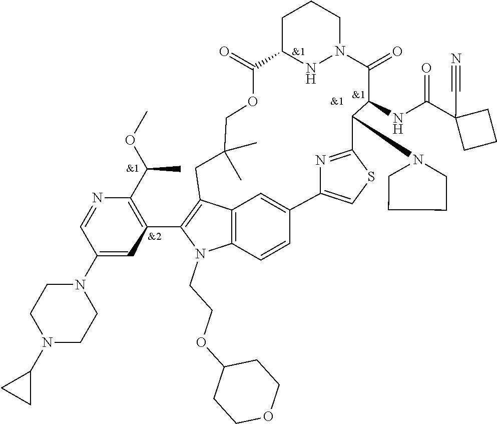 |
| A717 | 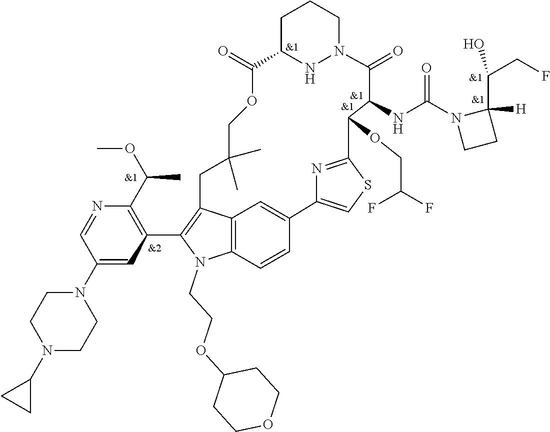 |
| A718 | 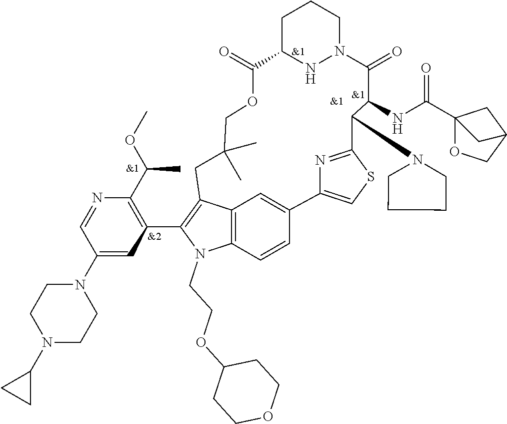 |
| A719 | 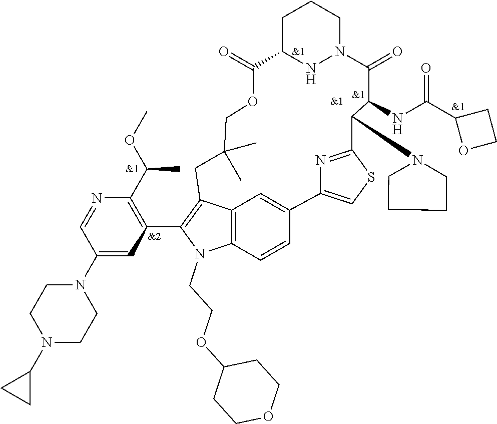 |
| A720 | 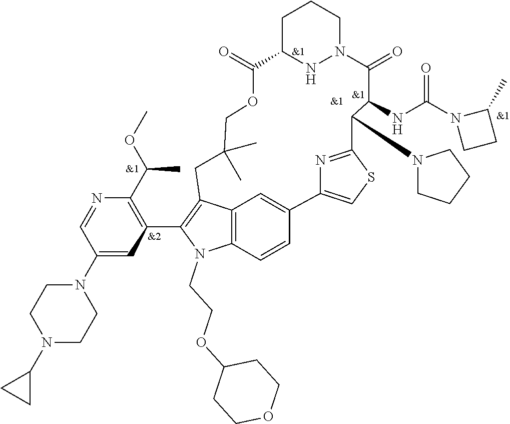 |
| A721 | 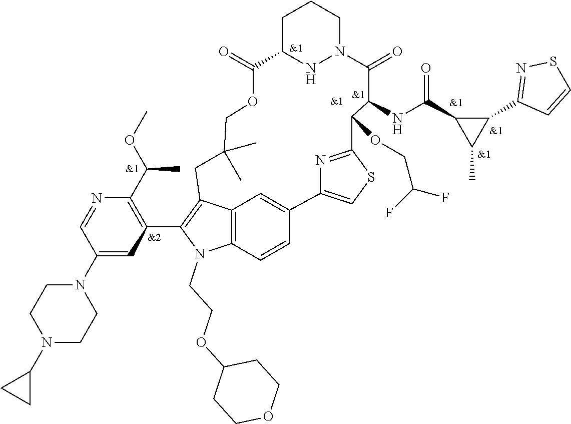 |
| A722 | 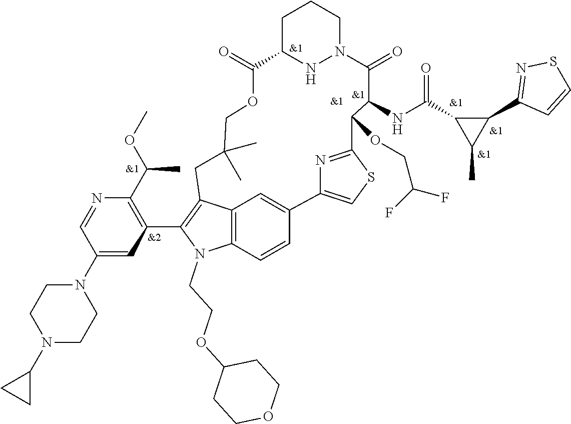 |
| A723 | 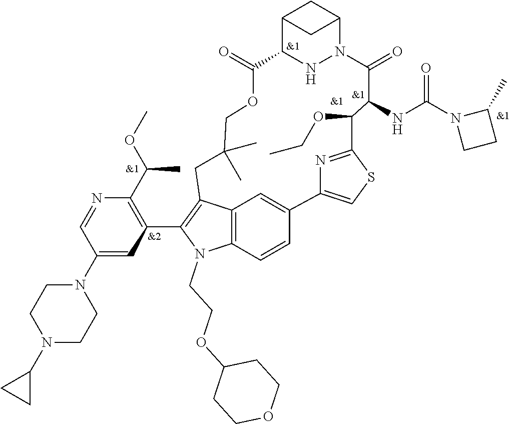 |
| A724 | 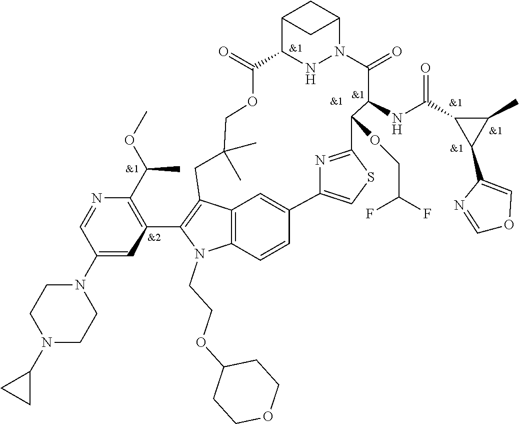 |
| A725 | 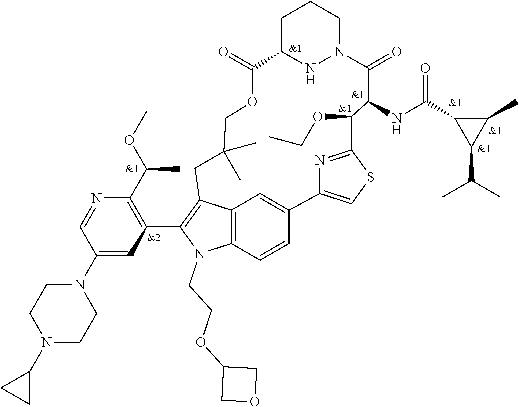 |
| A726 | 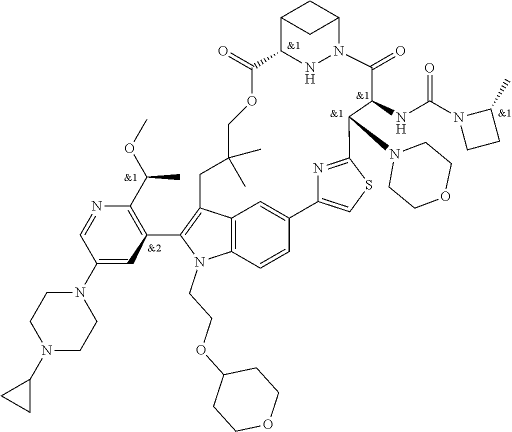 |
| A727 | 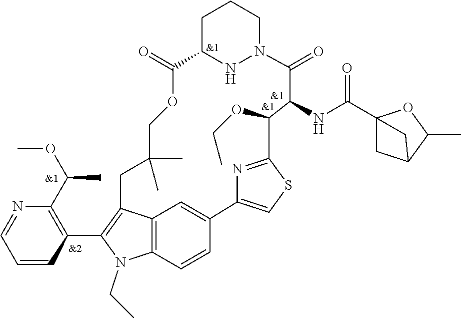 |
| A728 | 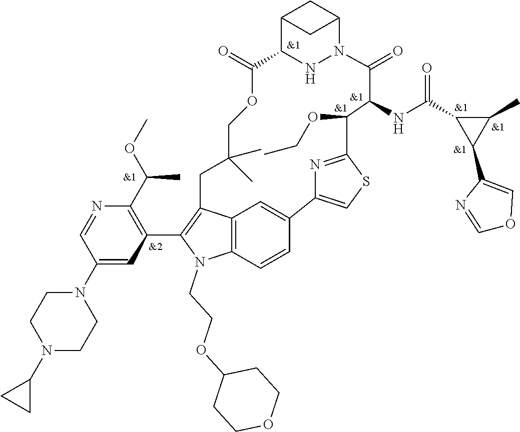 |
| A729 | 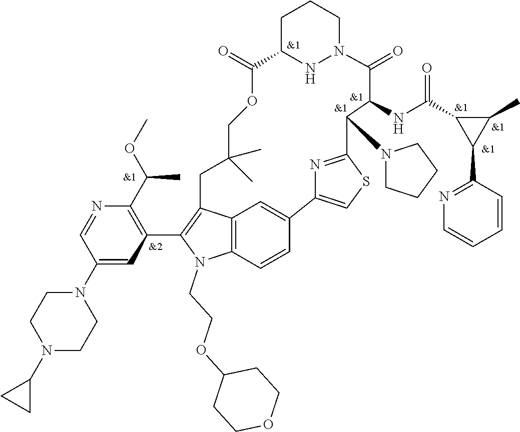 |
| A730 | 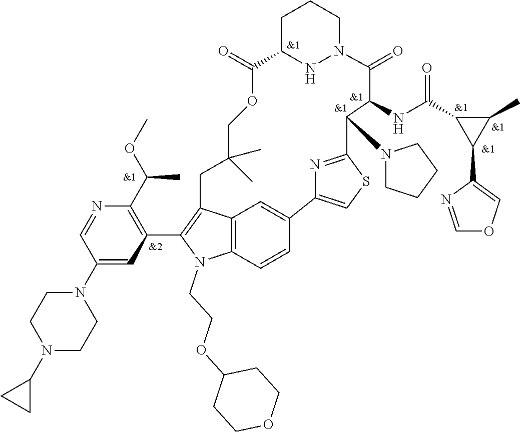 |
| A731 | 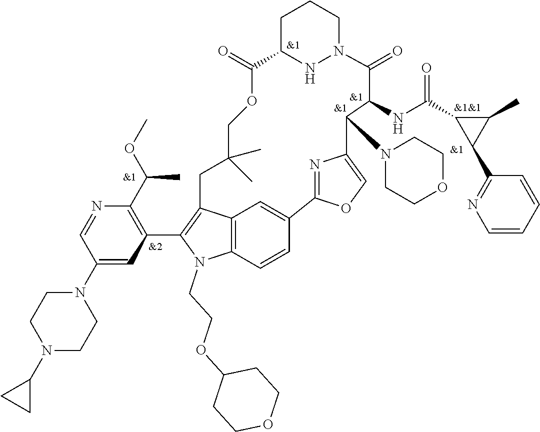 |
| A732 | 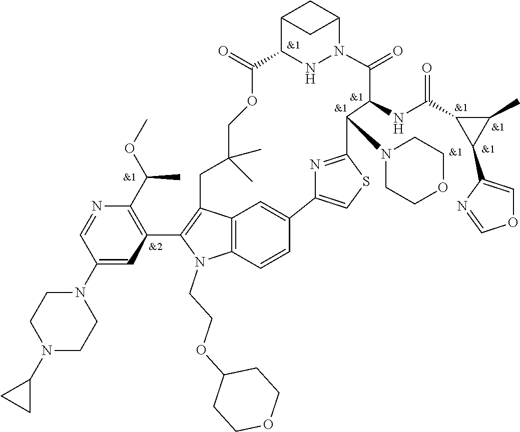 |
| A733 | 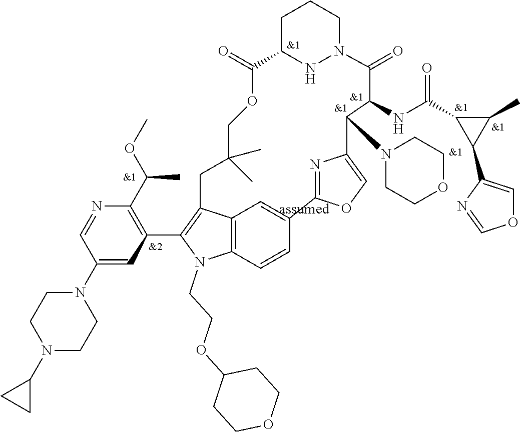 |
| A734 | 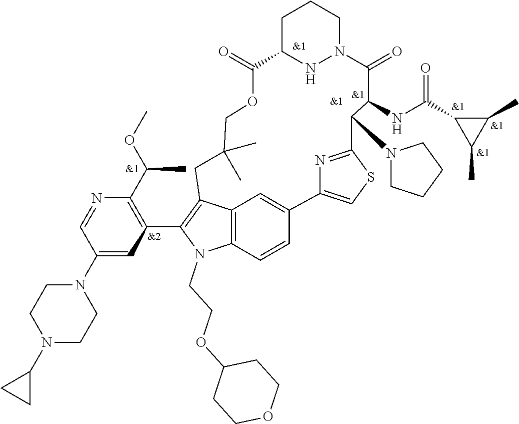 |
| A735 | 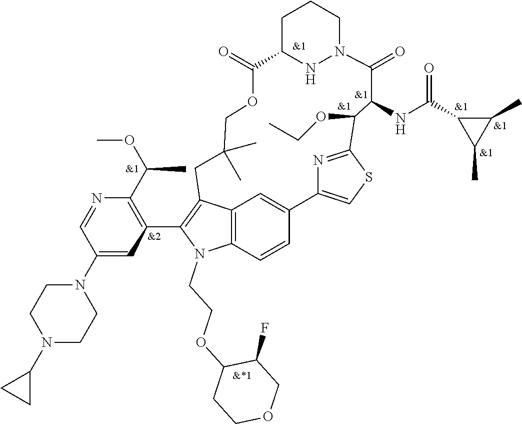 |
| A736 | 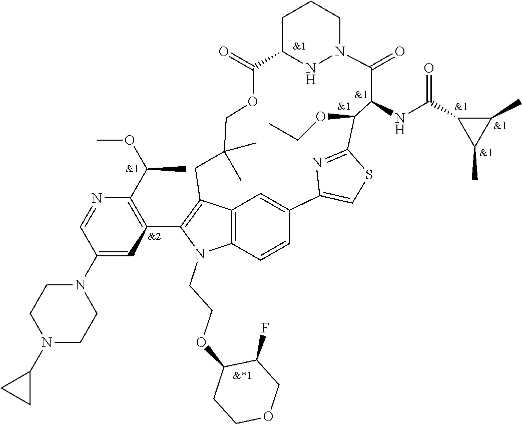 |
| A737 | 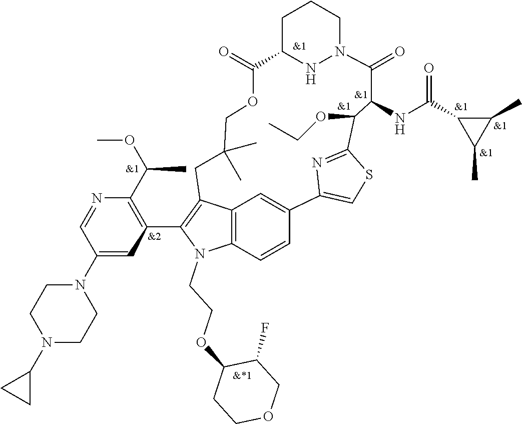 |
| A738 | 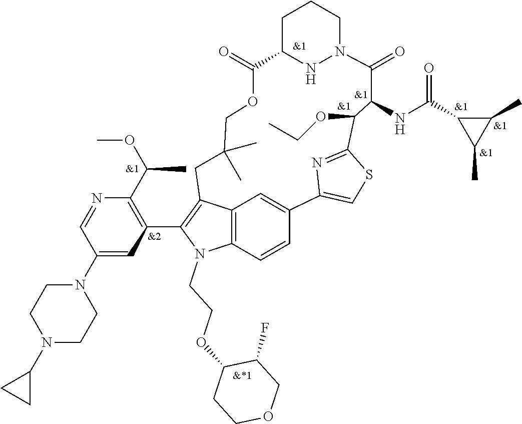 |
| A739 | 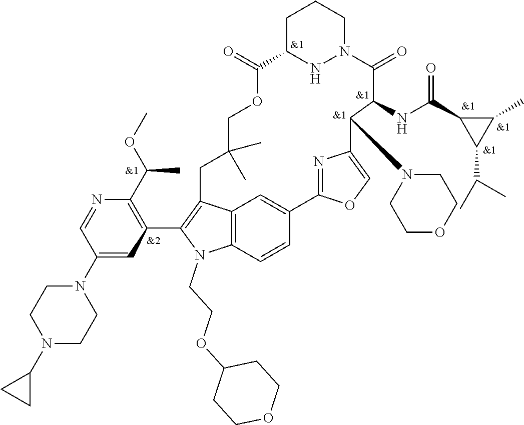 |
| A740 | 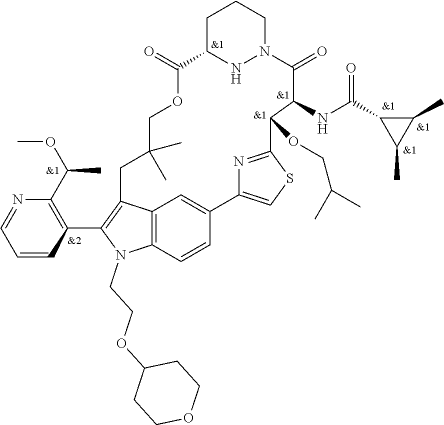 |
| A741 | 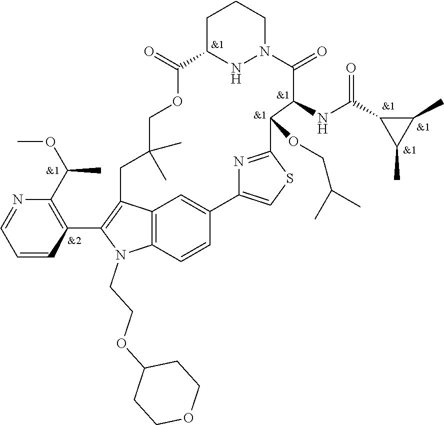 |
| A742 | 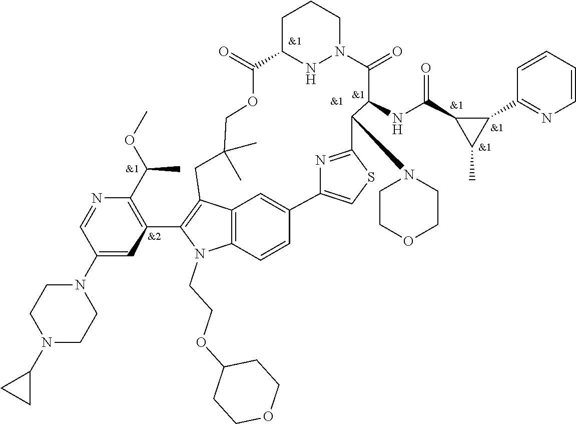 |
| A743 | 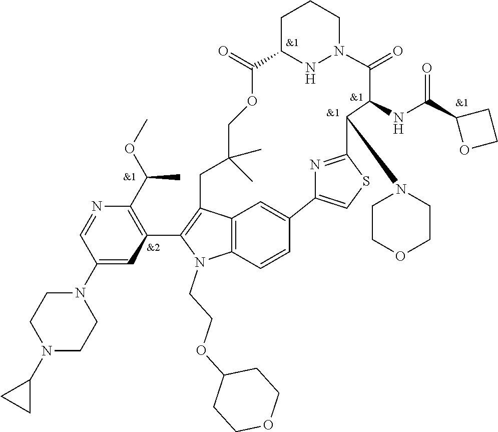 |
| A744 | 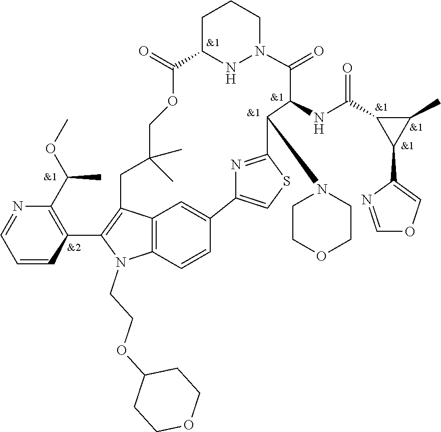 |
| A745 | 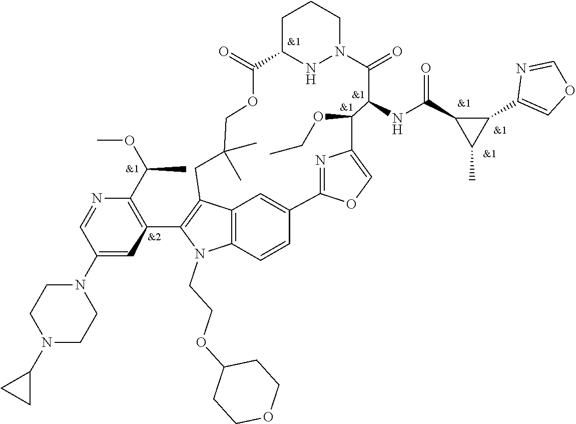 |
| A746 | 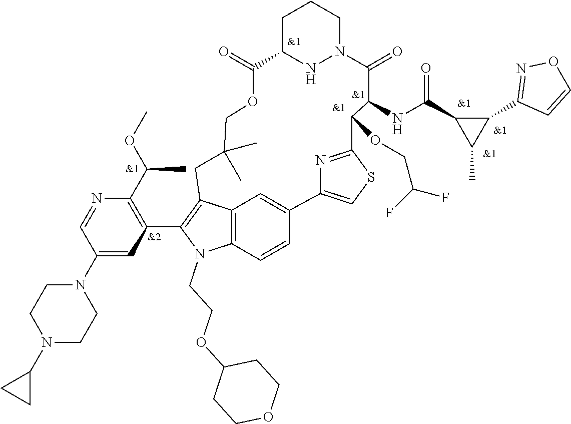 |
| A747 | 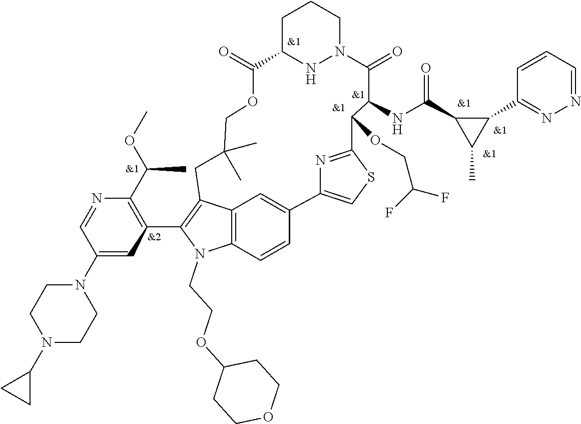 |
| A748 | 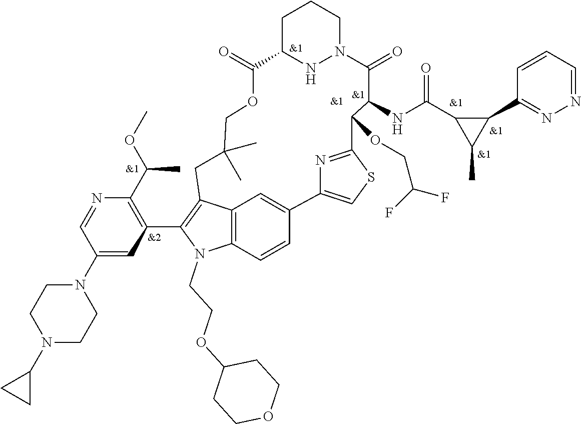 |
| A749 | 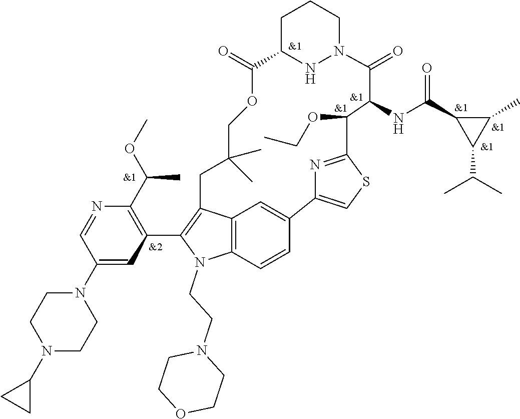 |
| A750 | 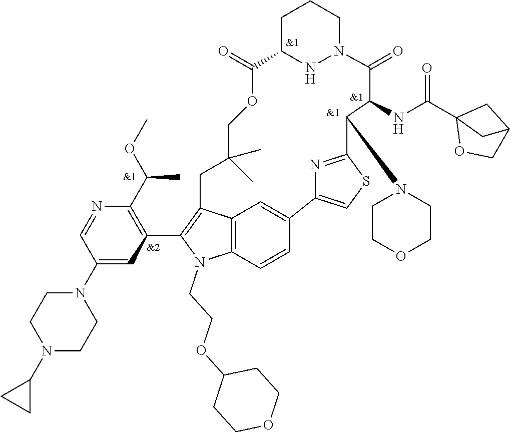 |
| A751 | 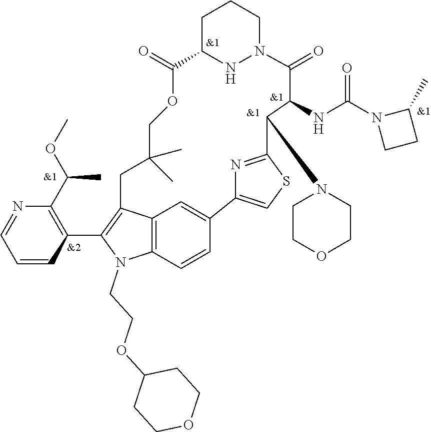 |
| A752 | 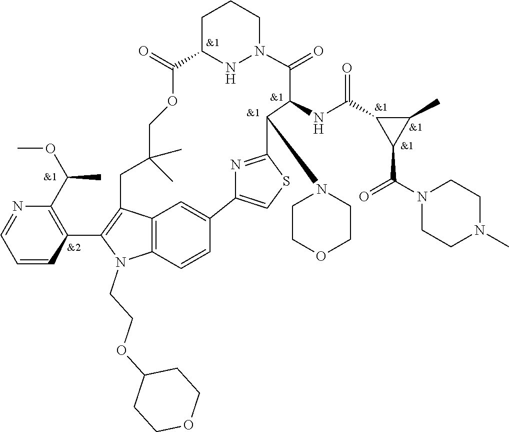 |
| A753 | 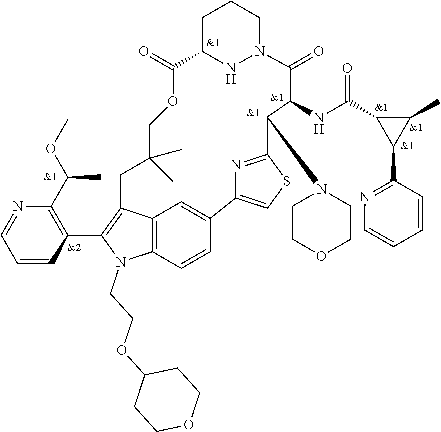 |
| A754 | 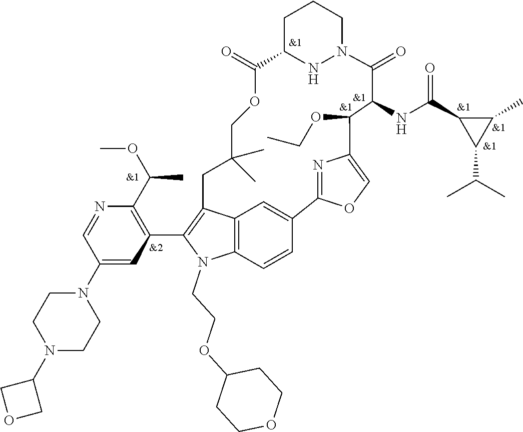 |
| A755 | 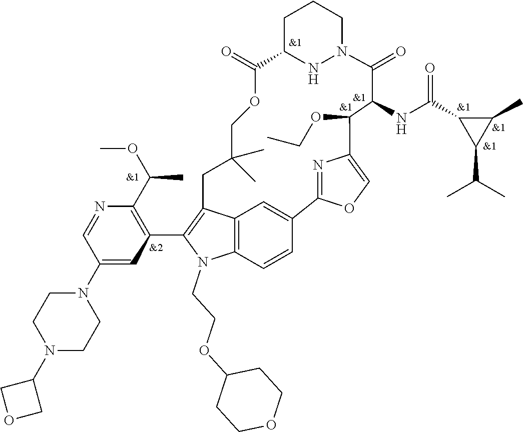 |
| A756 | 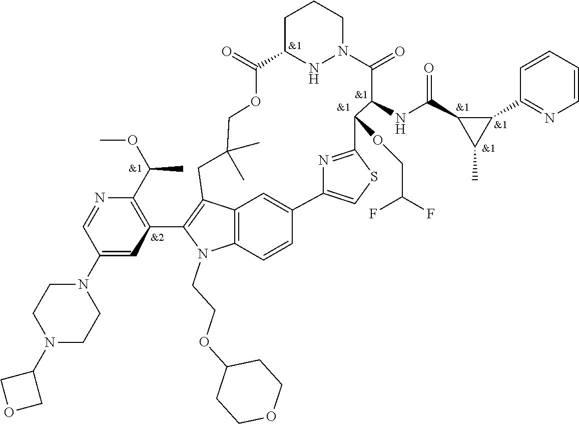 |
| A757 | 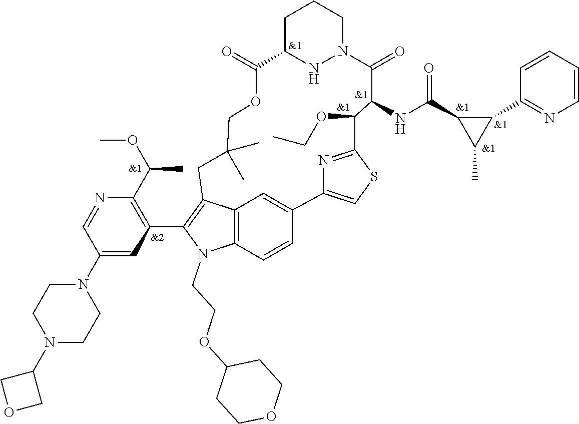 |
| A758 | 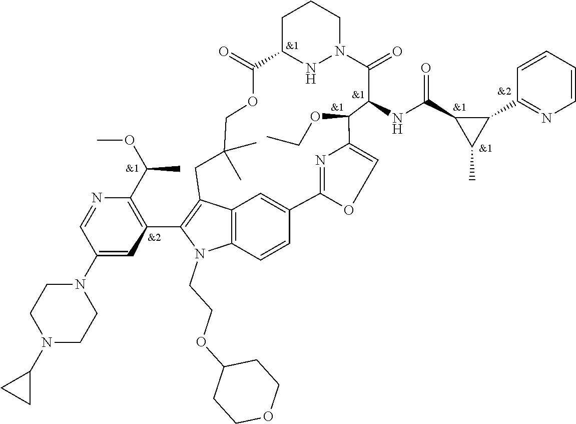 |
| A759 | 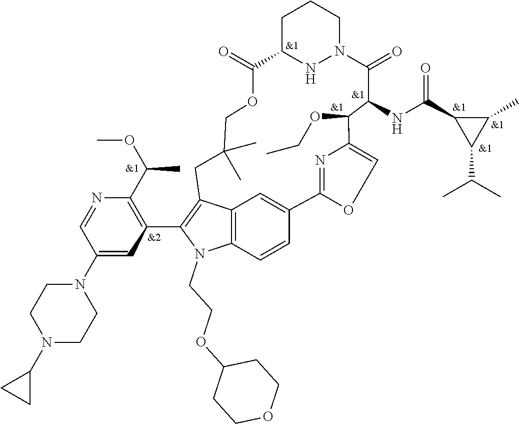 |
| A760 | 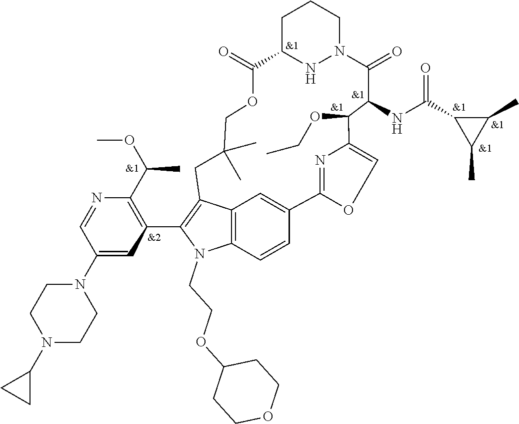 |
| A761 | 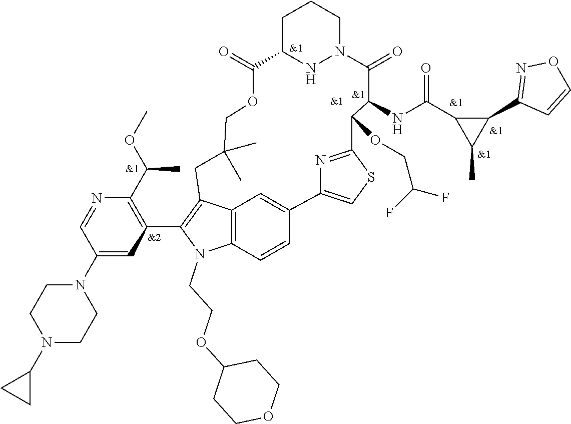 |
| A762 | 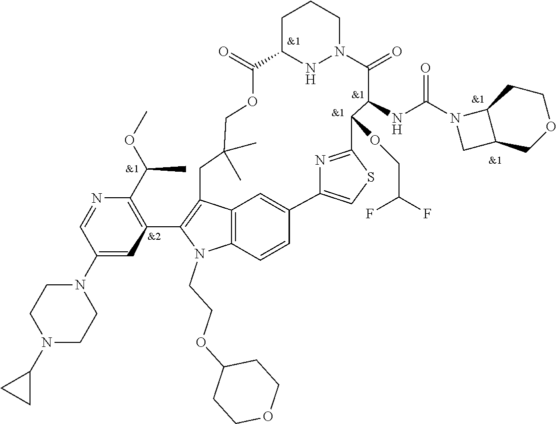 |
| A763 | 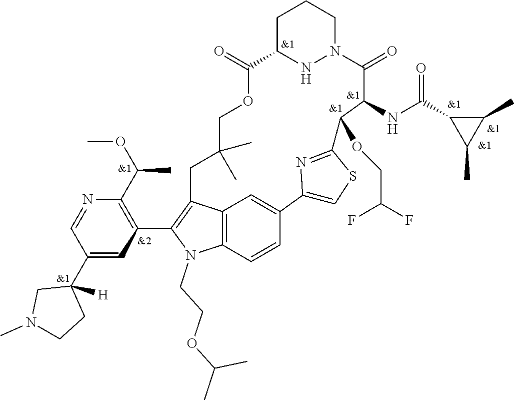 |
| A764 | 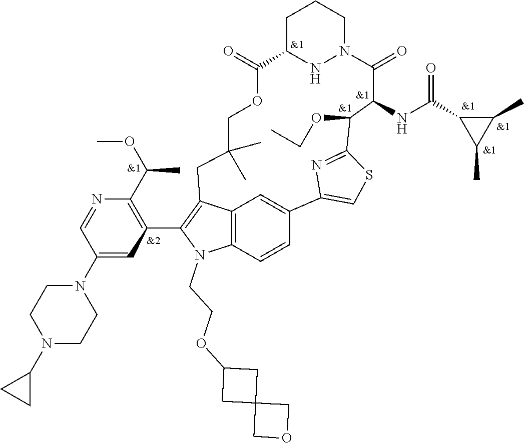 |
| A765 | 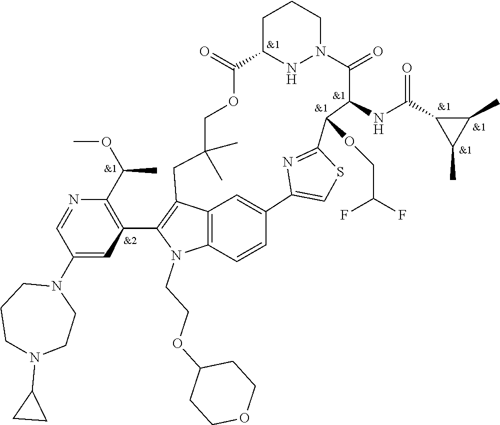 |
| A766 | 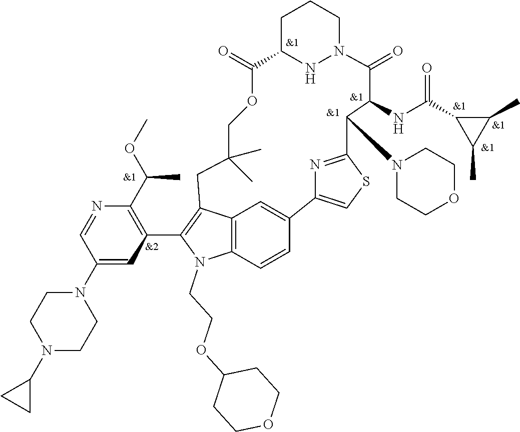 |
| A767 | 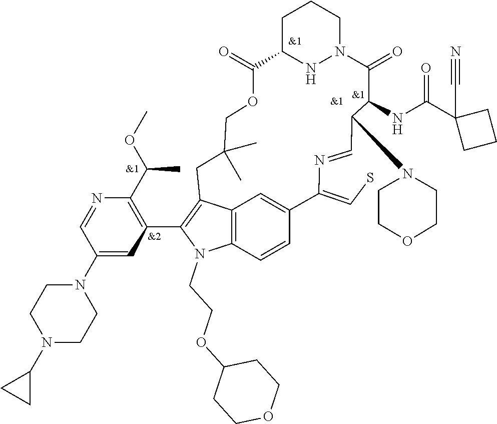 |
| A768 | 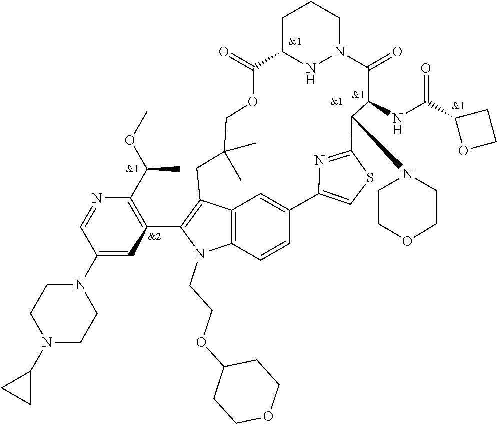 |
| A769 | 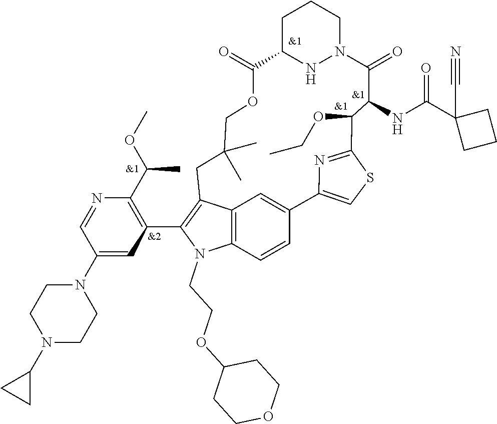 |
| A770 | 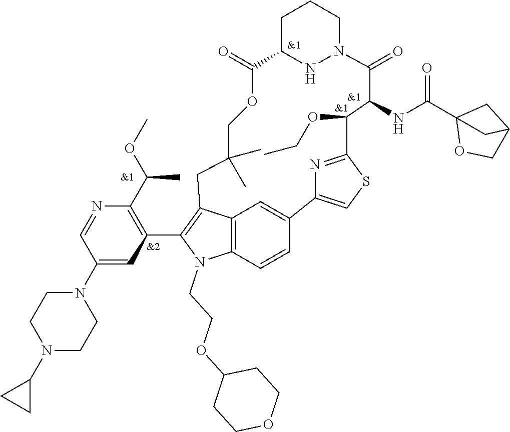 |
| A771 | 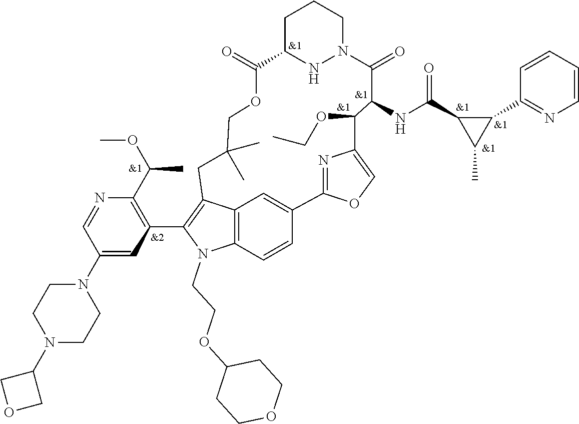 |
| A772 | 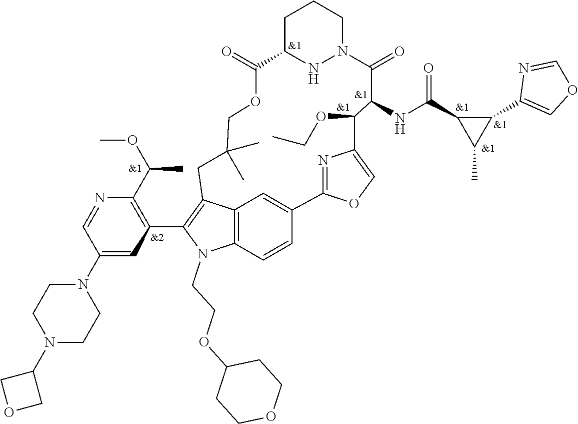 |
| A773 | 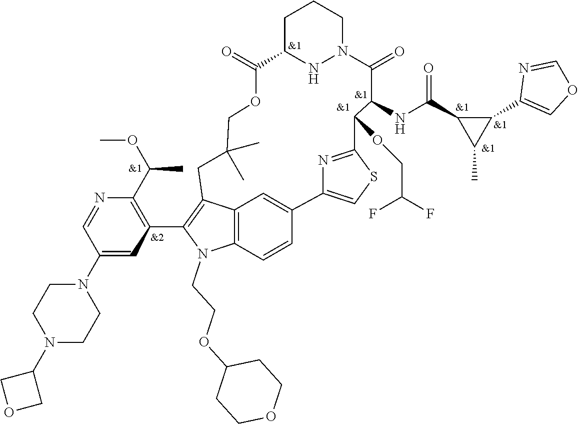 |
| A774 | 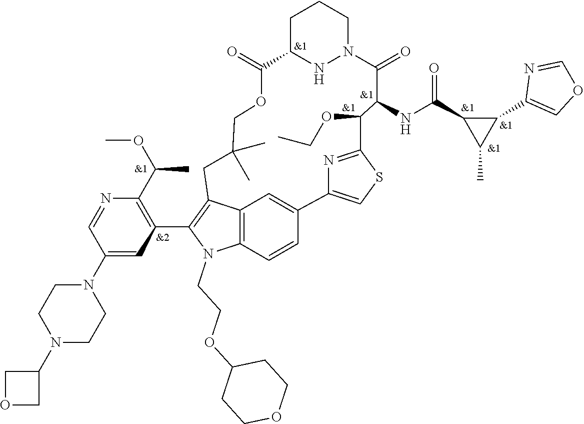 |
| A775 | 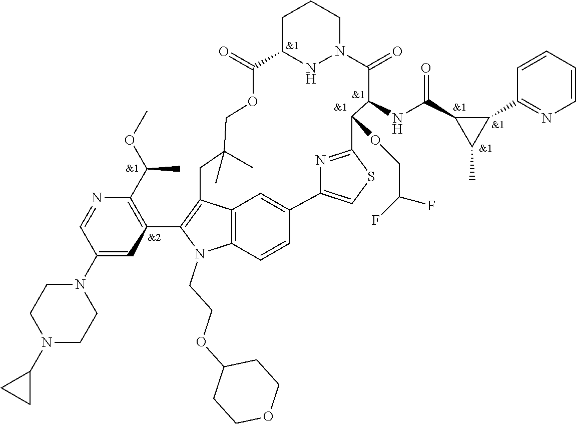 |
| A776 | 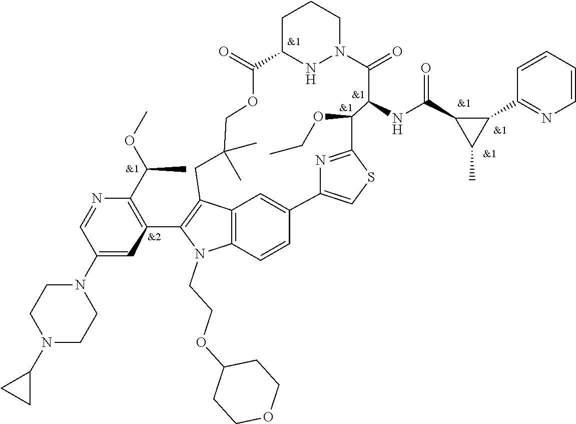 |
| A777 | 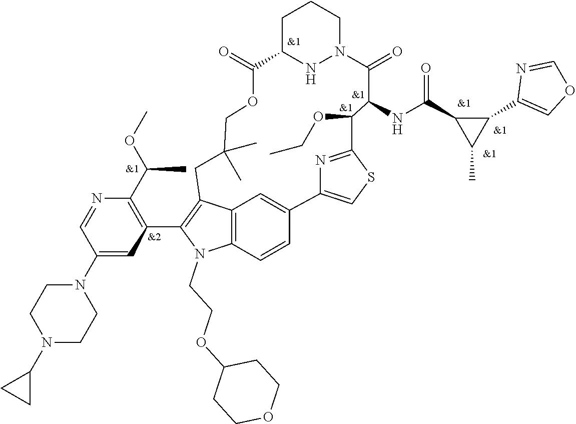 |
| A778 | 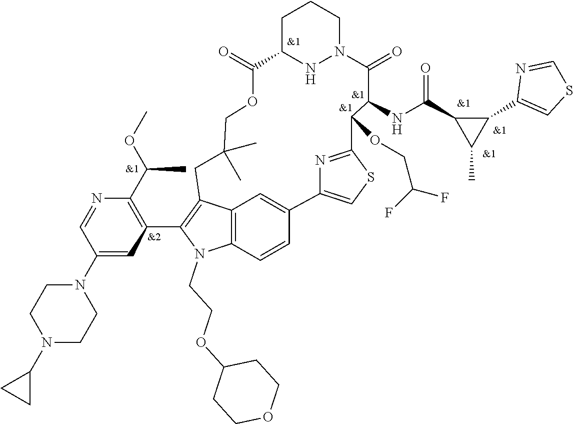 |
| A779 | 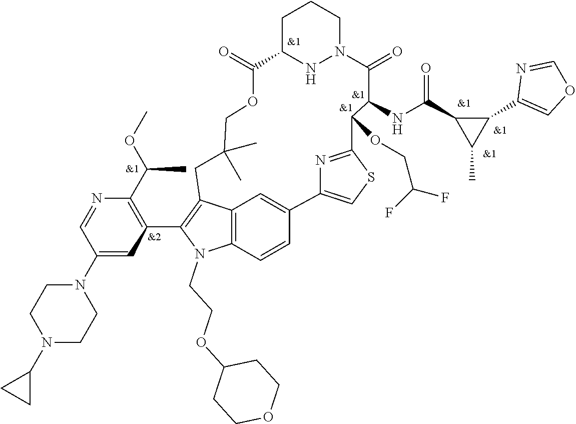 |
| A780 | 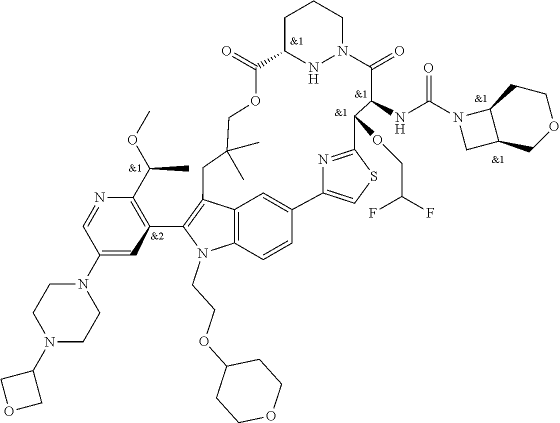 |
| A781 | 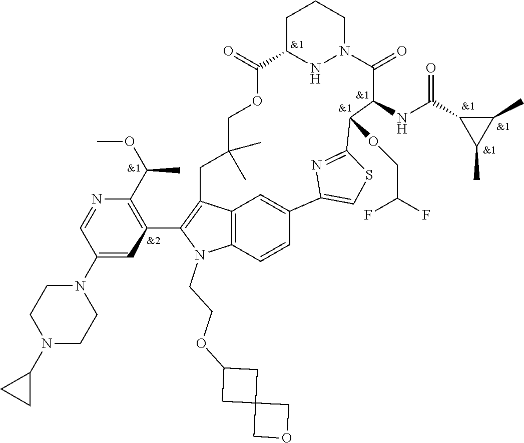 |
| A782 | 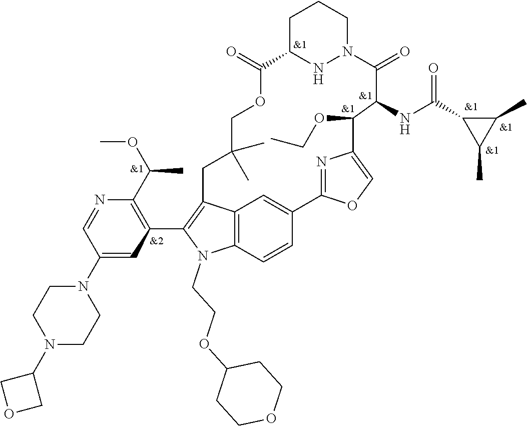 |
| A783 | 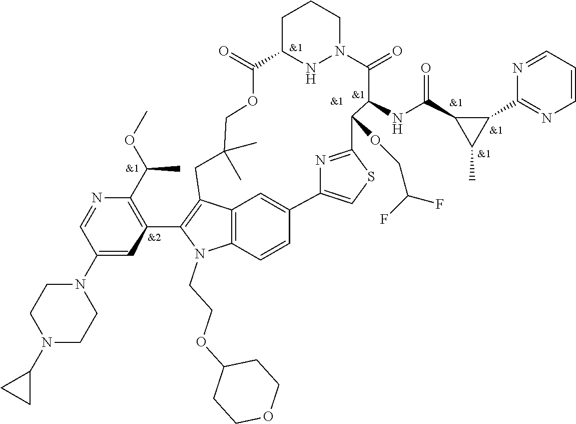 |
| A784 | 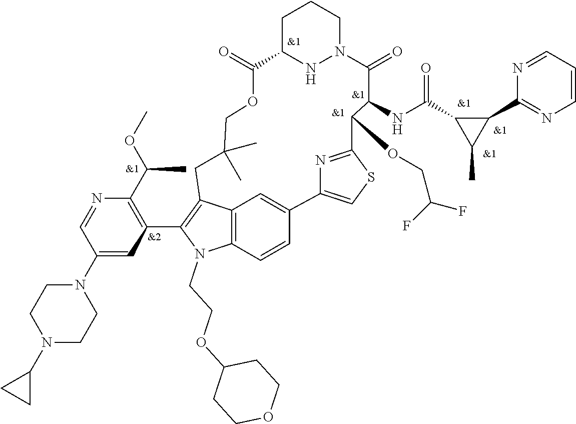 |
| A785 | 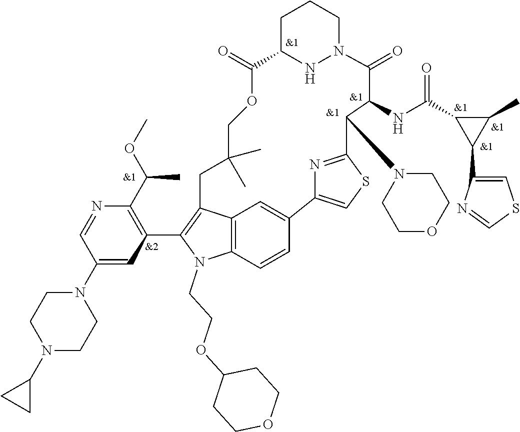 |
| A786 | 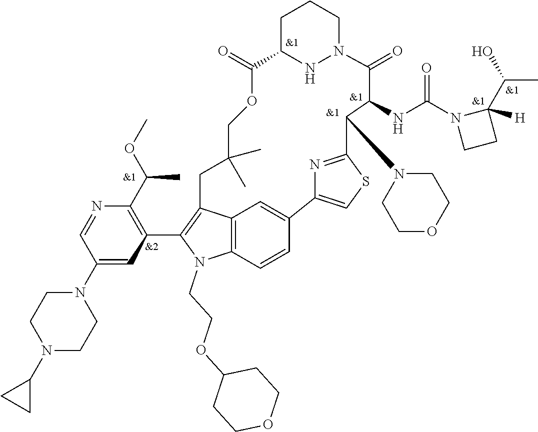 |
| A787 | 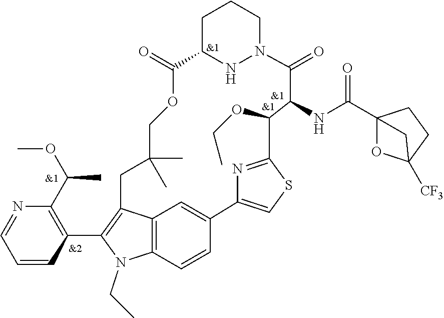 |
| A788 | 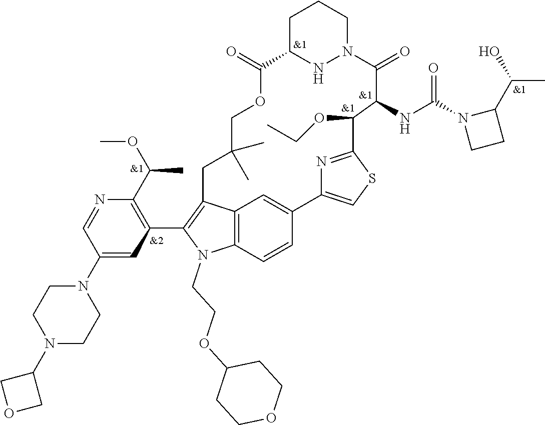 |
| A789 | 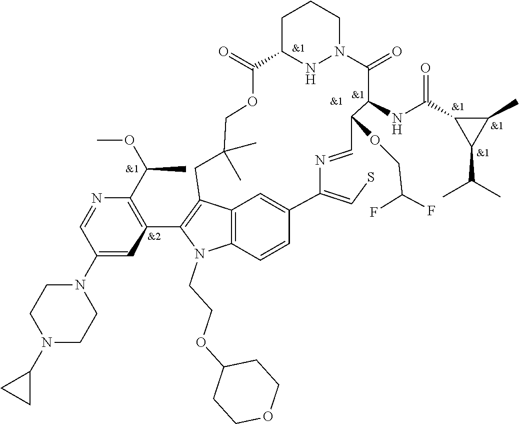 |
| A790 | 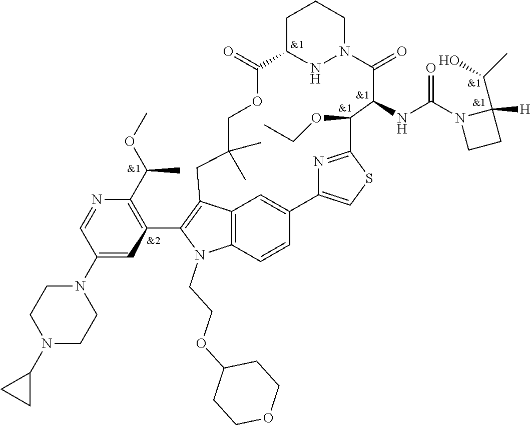 |
| A791 | 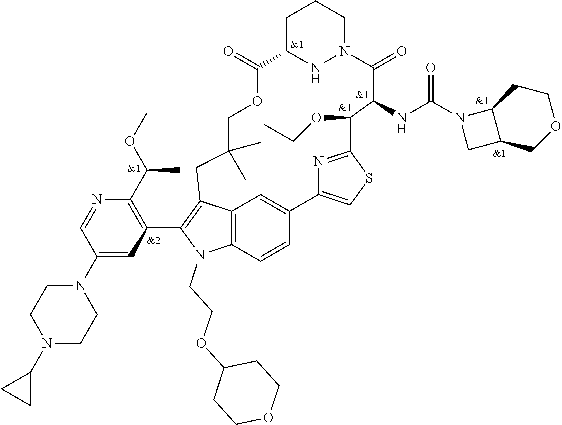 |
| A792 | 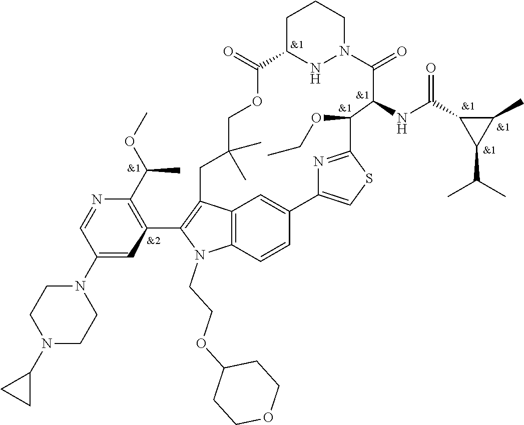 |
| A793 | 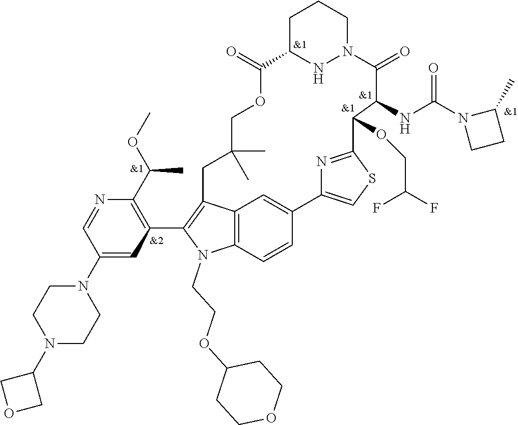 |
| A794 | 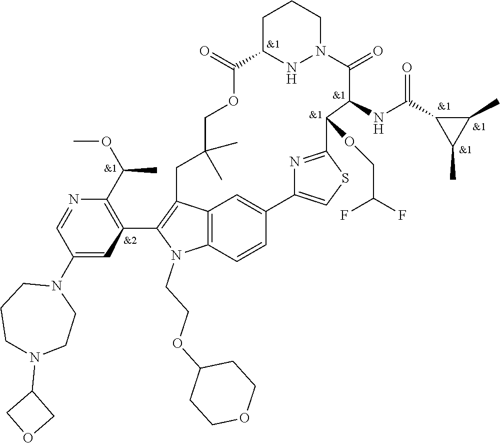 |
| A795 | 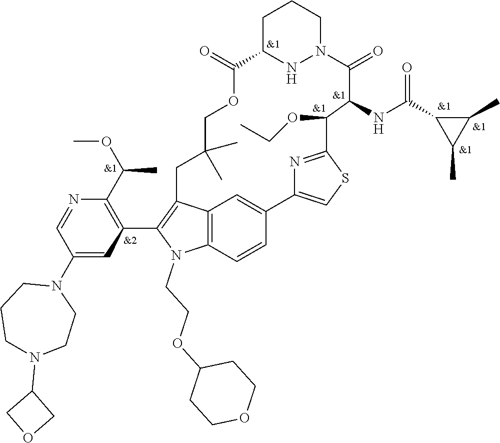 |
| A796 | 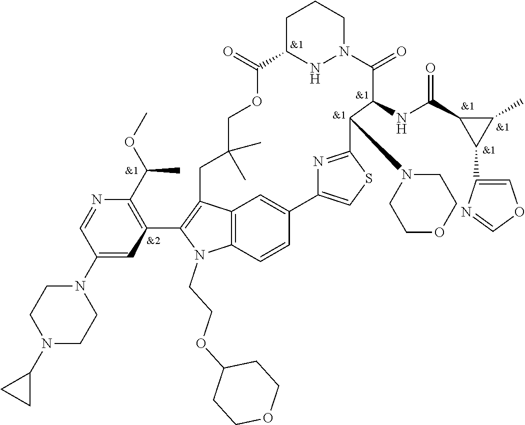 |
| A797 | 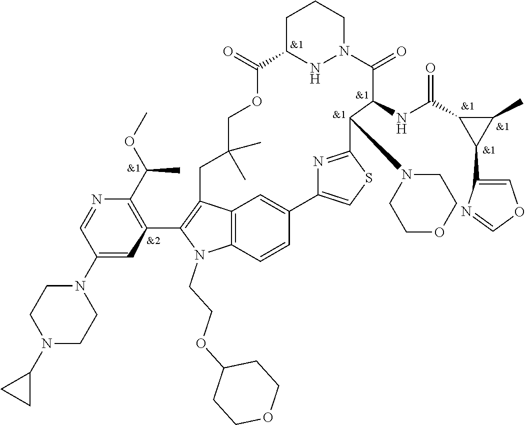 |
| A798 | 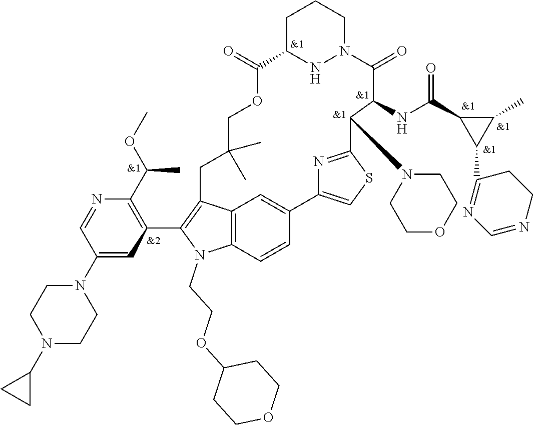 |
| A799 | 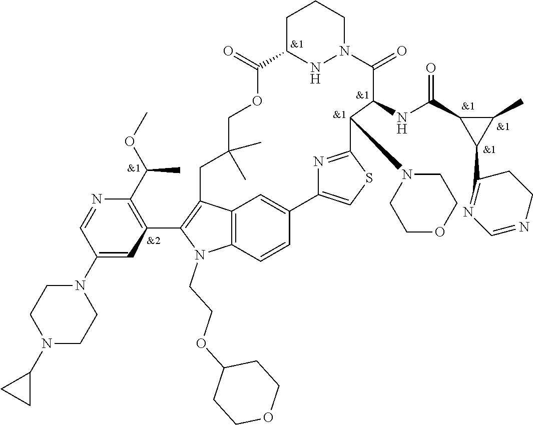 |
| A800 | 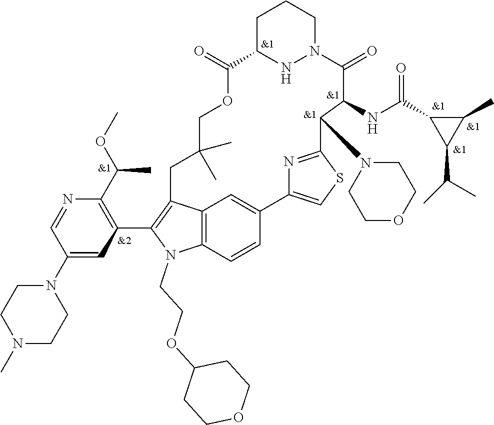 |
| A801 | 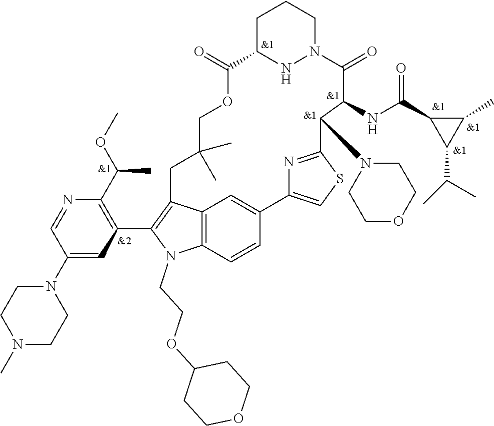 |
| A802 | 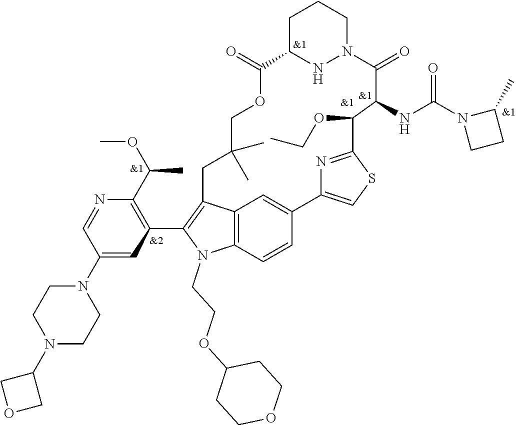 |
| A803 | 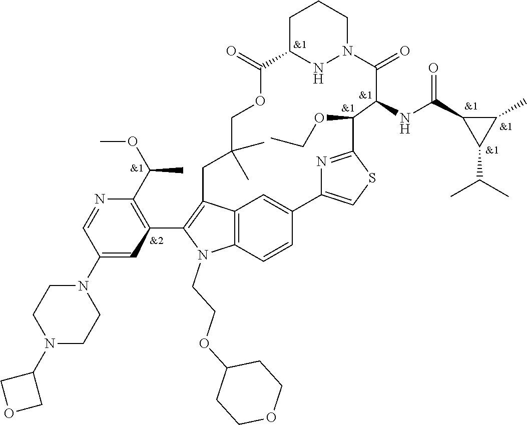 |
| A804 | 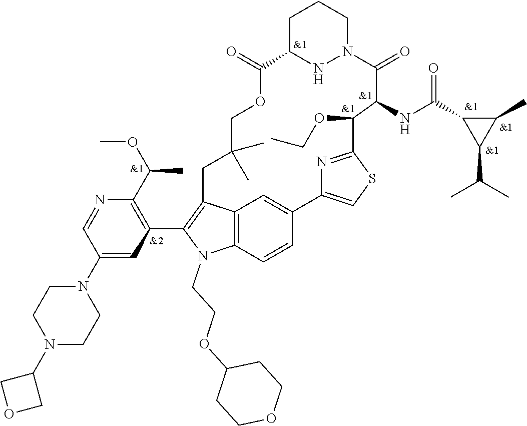 |
| A805 | 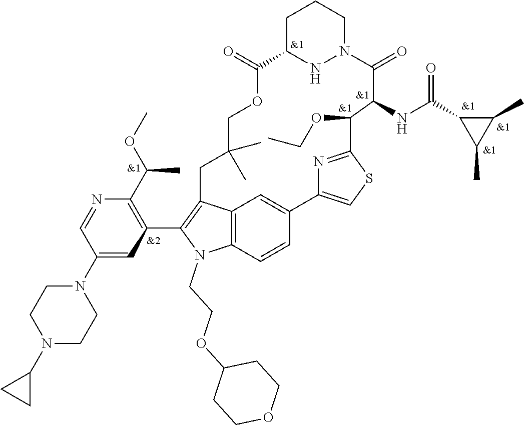 |
| A806 | 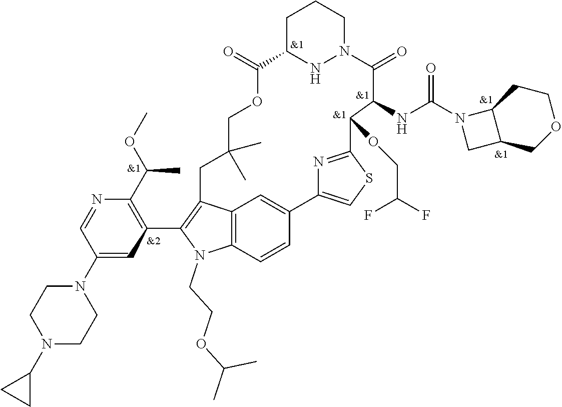 |
| A807 | 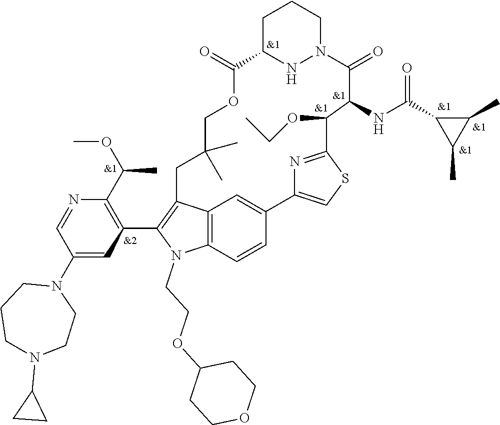 |
| A808 | 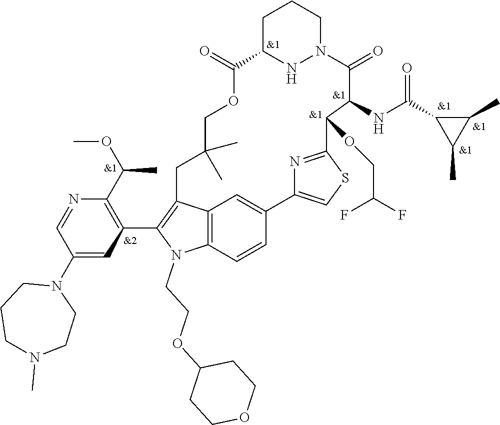 |
| A809 | 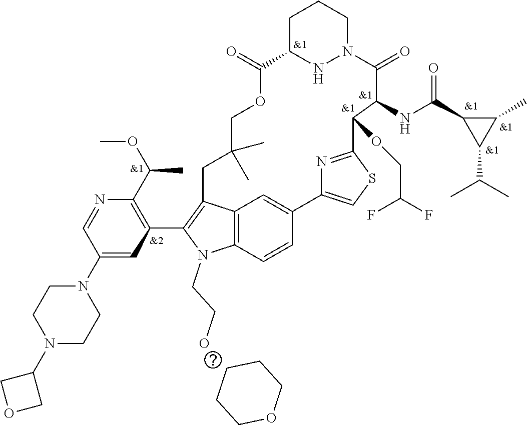 |
| A810 | 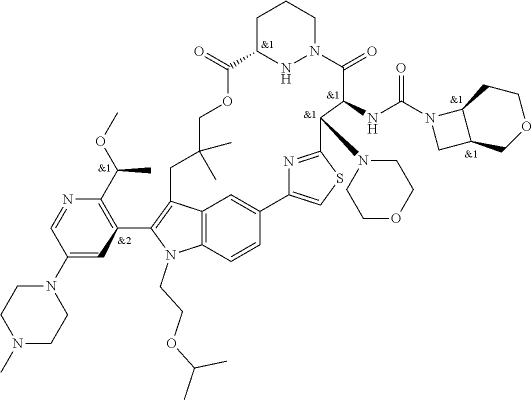 |
| A811 | 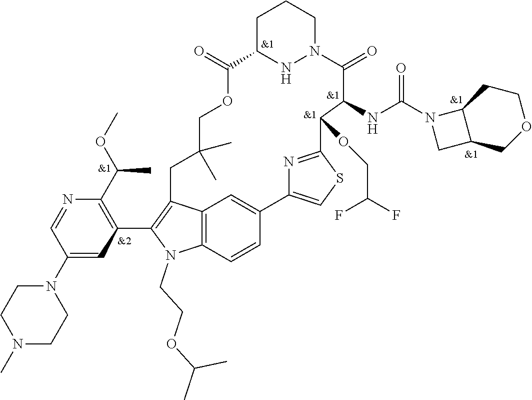 |
| A812 | 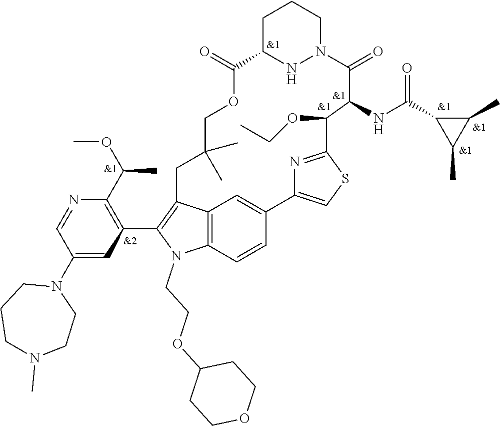 |
| A813 | 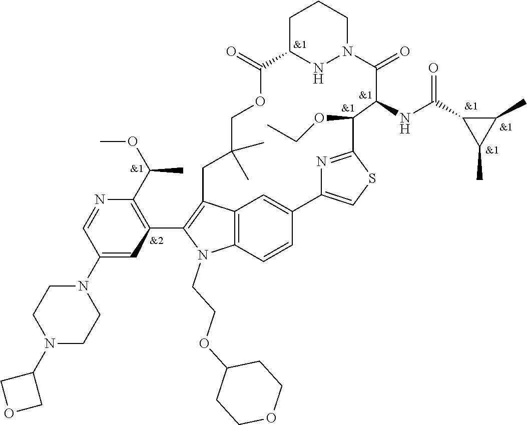 |
| A814 | 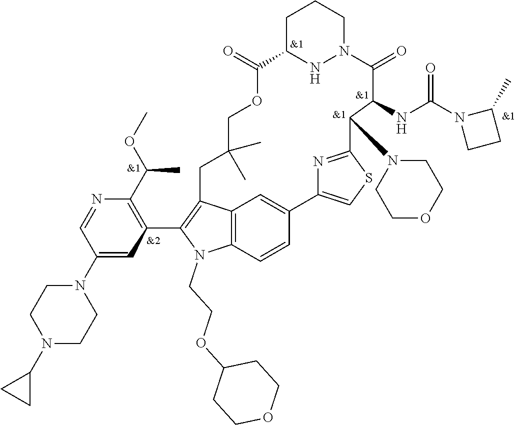 |
| A815 | 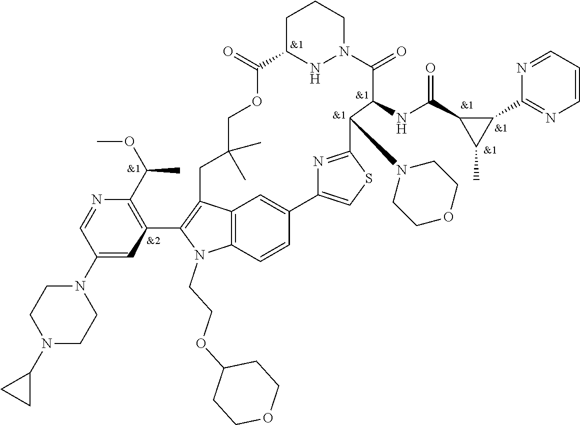 |
| A816 | 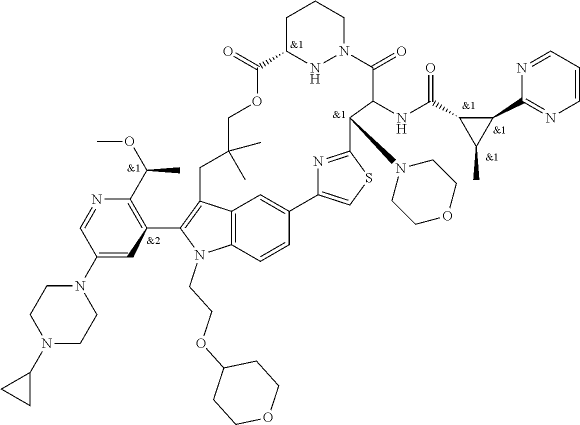 |
| A817 | 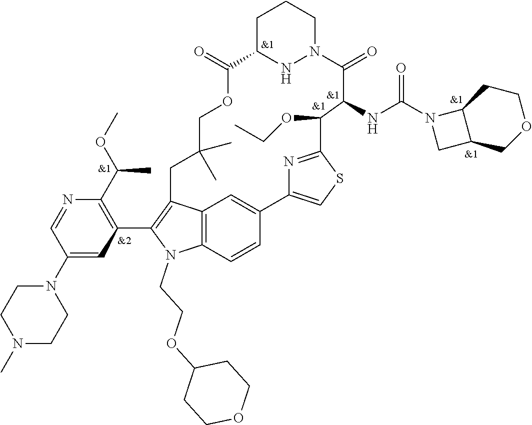 |
| A818 | 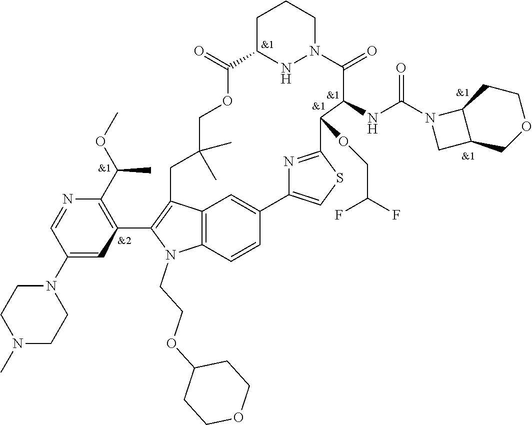 |
| A819 | 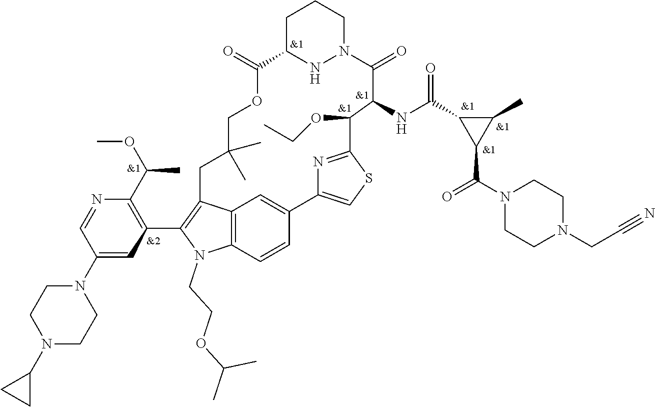 |
| A820 | 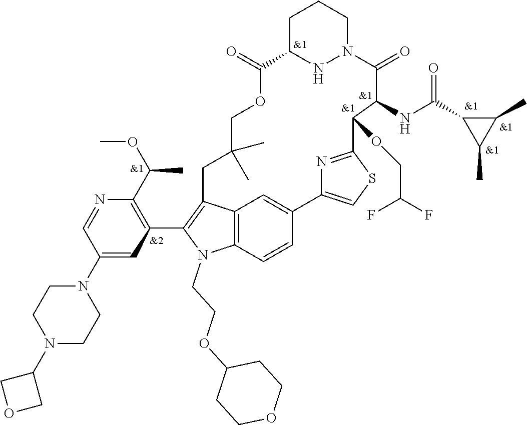 |
| A821 | 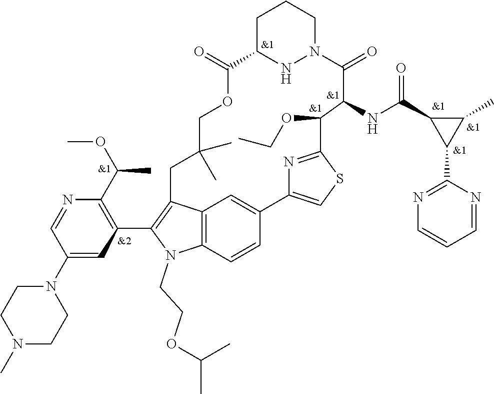 |
| A822 | 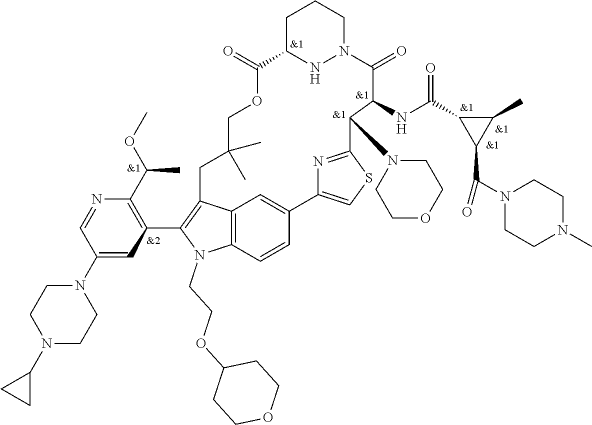 |
| A823 | 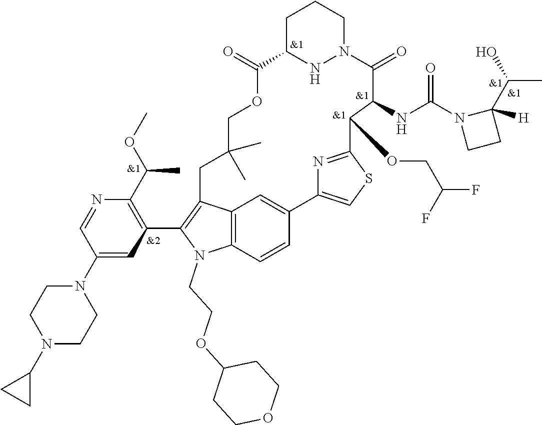 |
| A824 | 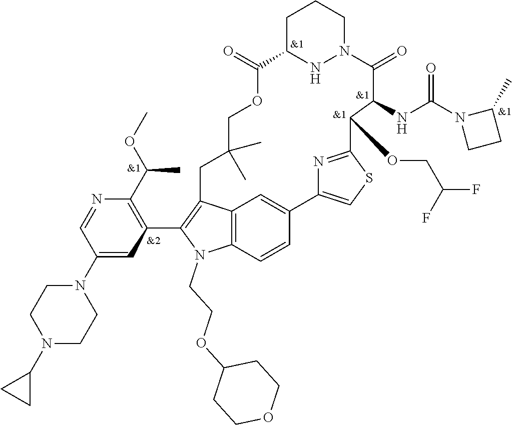 |
| A825 | 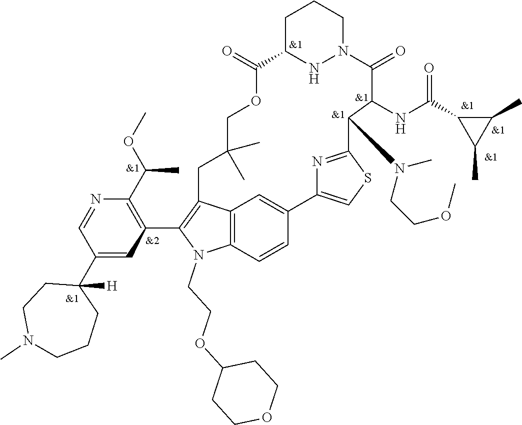 |
| A826 | 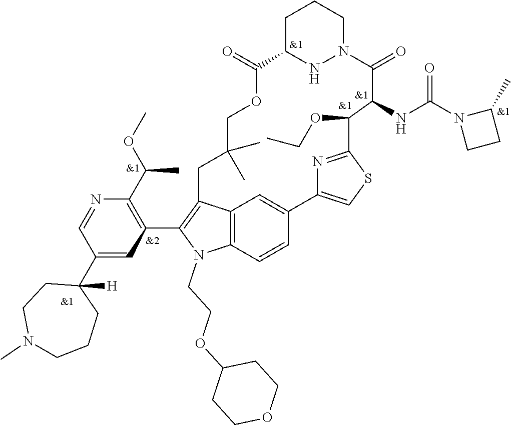 |
| A827 | 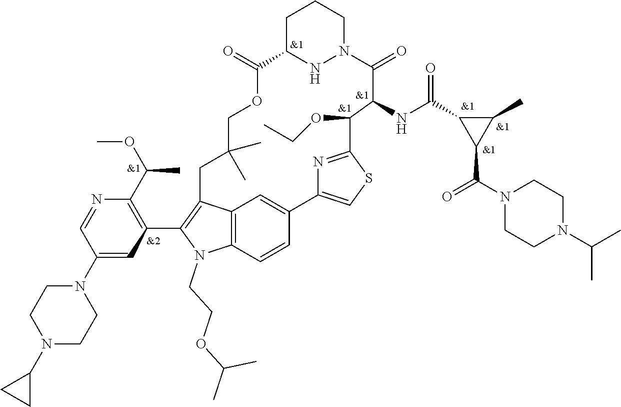 |
| A828 | 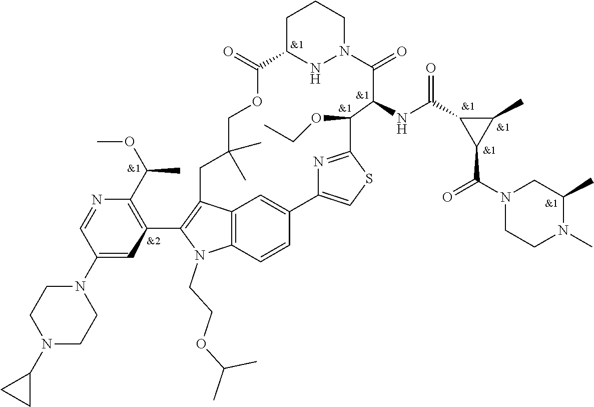 |
| A829 | 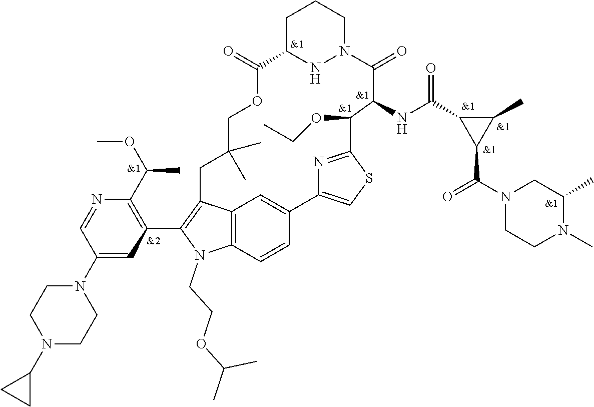 |
| A830 | 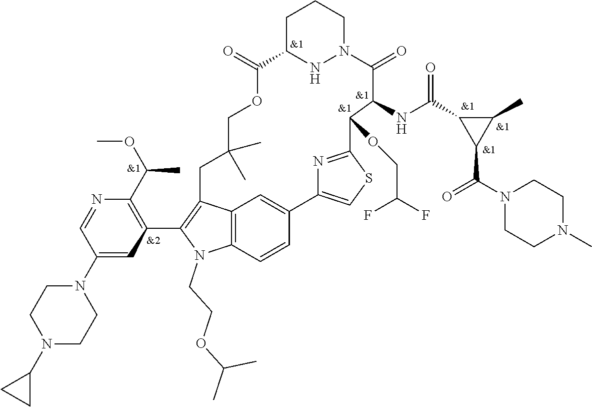 |
| A831 | 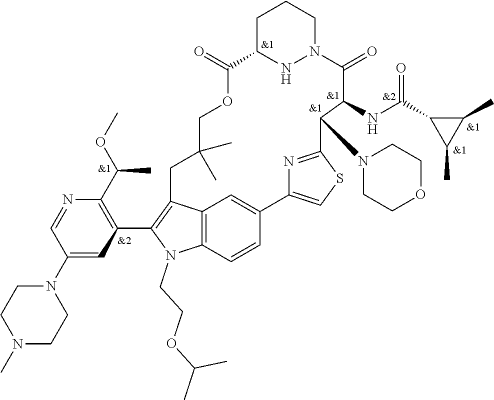 |
| A832 | 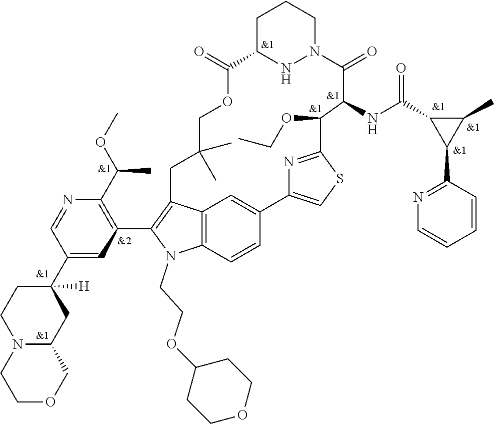 |
| A833 | 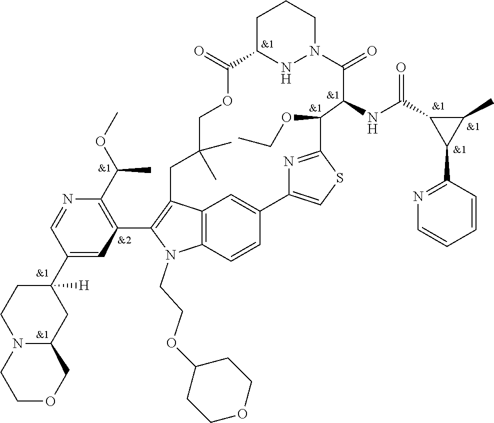 |
| A834 | 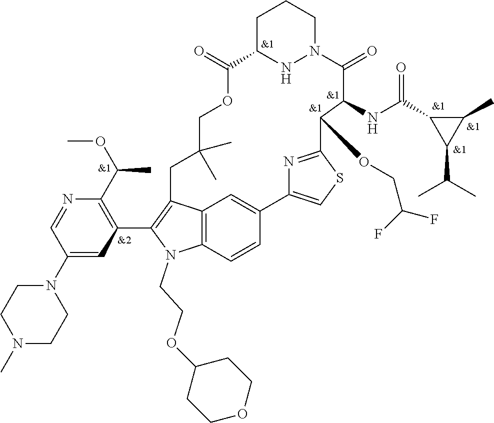 |
| A835 | 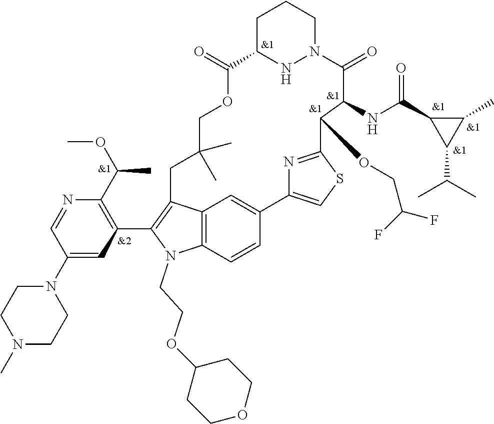 |
| A836 | 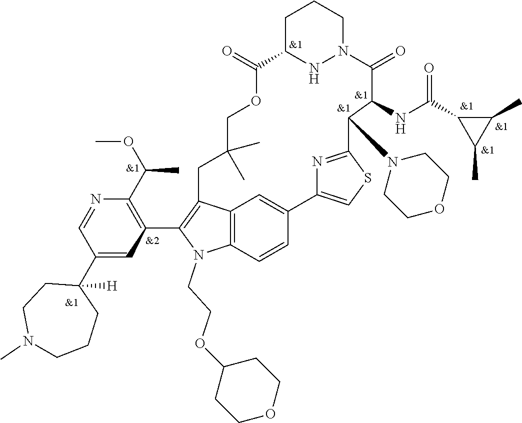 |
| A837 | 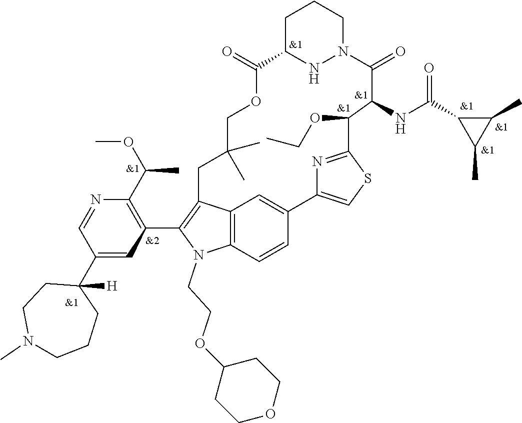 |
| A838 | 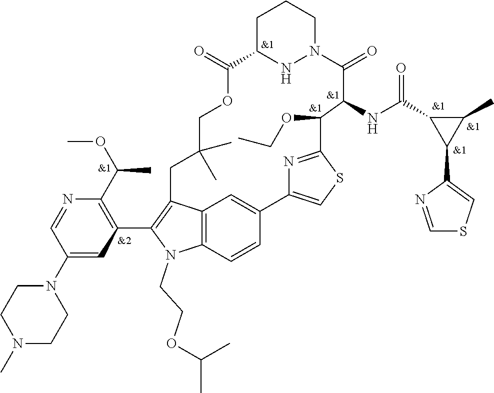 |
| A839 | 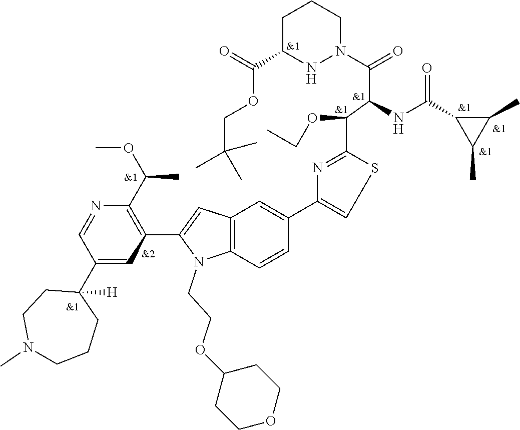 |
| A840 | 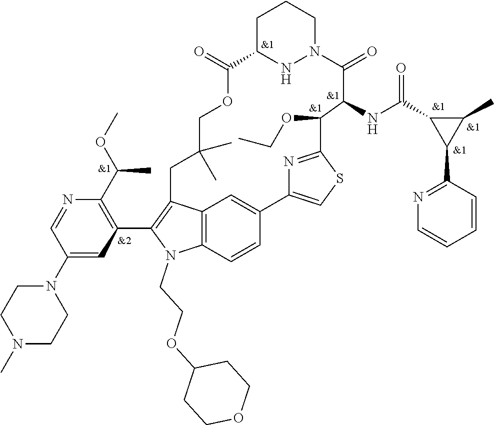 |
| A841 | 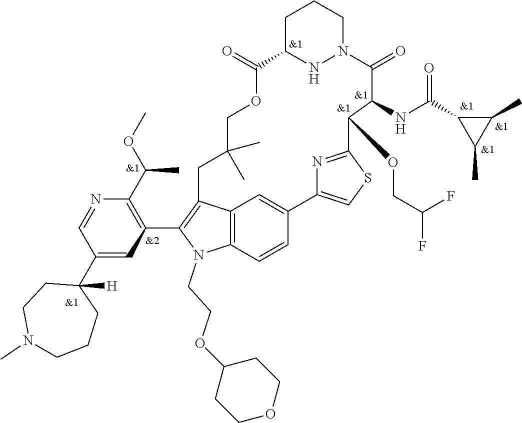 |
| A842 | 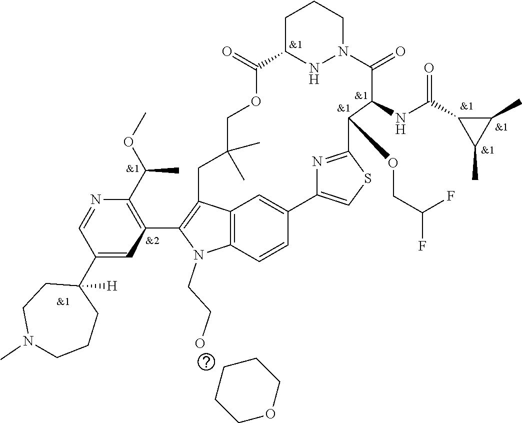 |
| A843 | 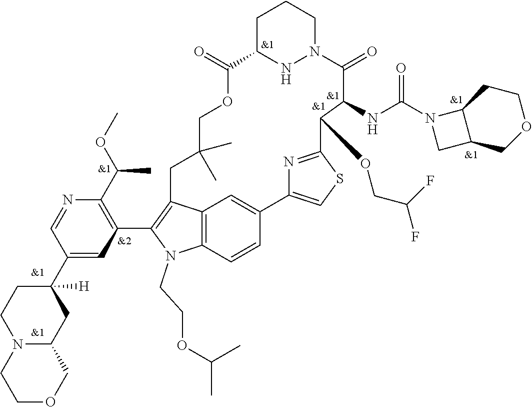 |
| A844 | 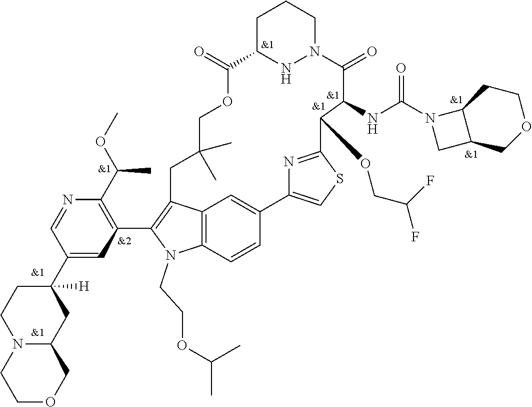 |
| A845 | 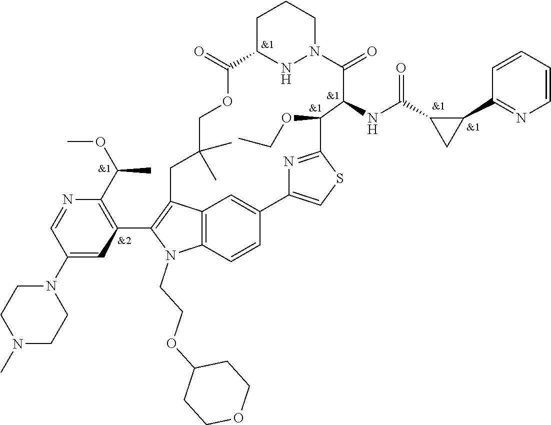 |
| A846 | 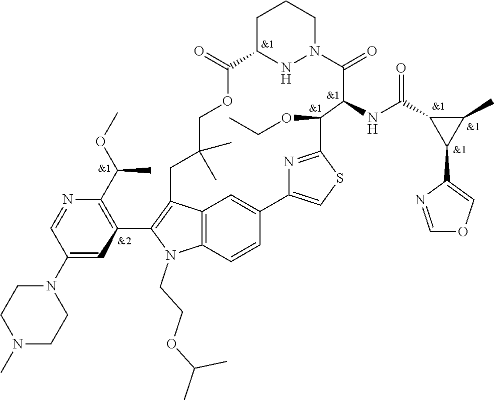 |
| A847 | 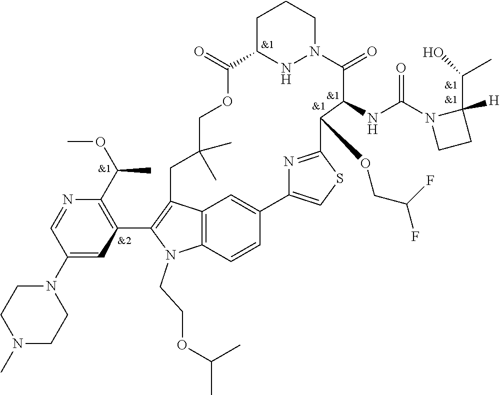 |
| A848 | 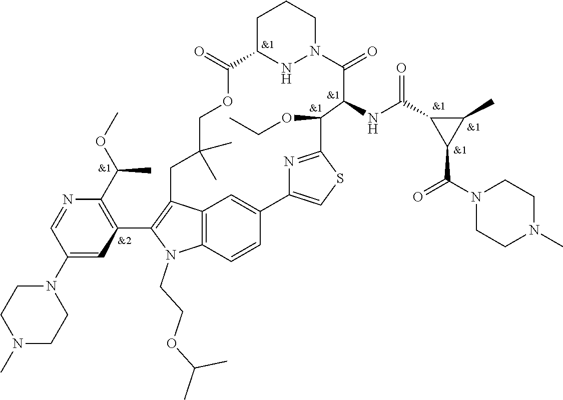 |
| A849 | 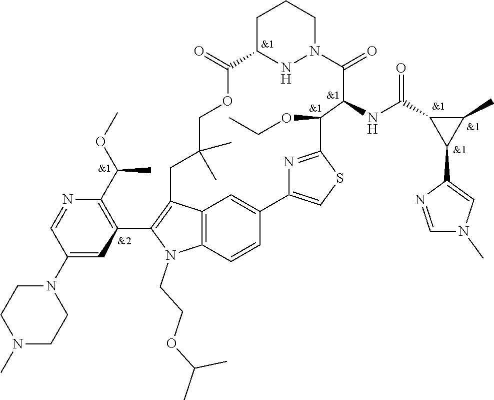 |
| A850 | 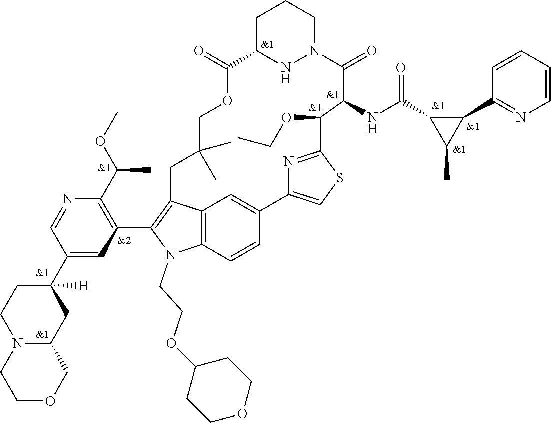 |
| A851 | 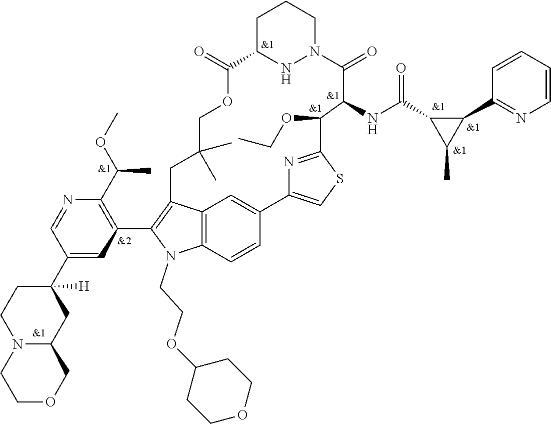 |
| A852 | 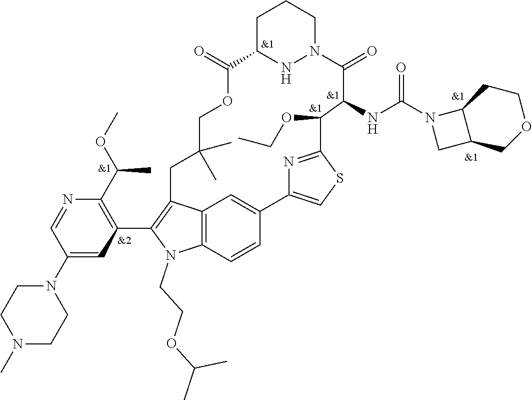 |
| A853 | 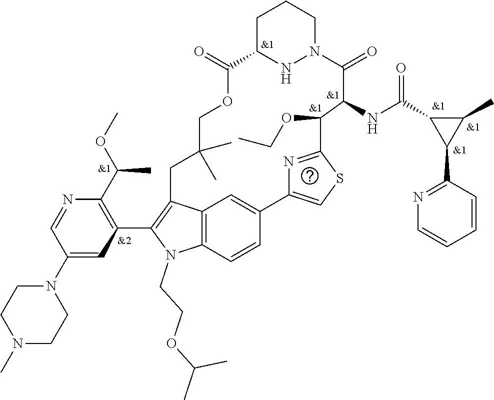 |
| A854 | 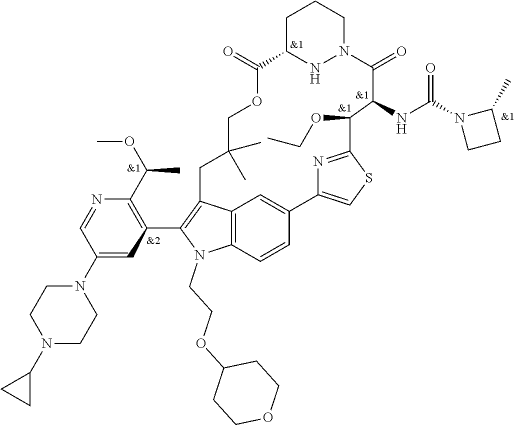 |
| A855 | 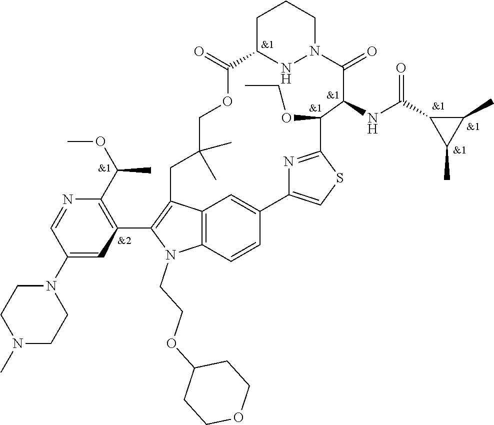 |
| A856 | 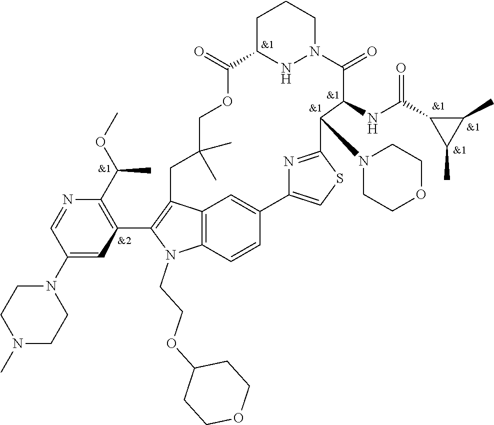 |
| A857 | 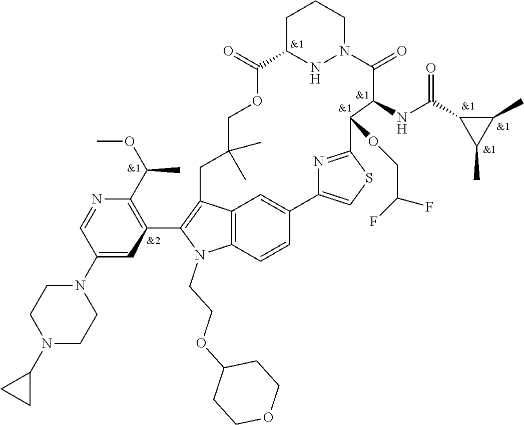 |
| A858 | 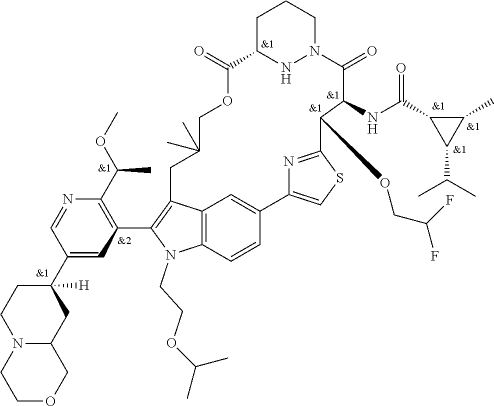 |
| A859 | 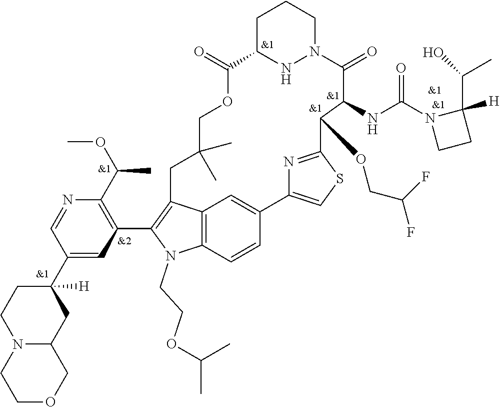 |
| A860 | 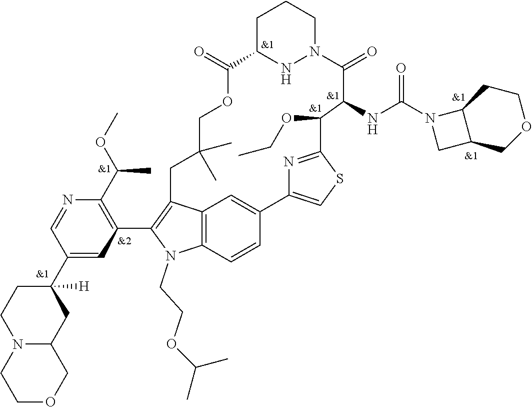 |
| A861 | 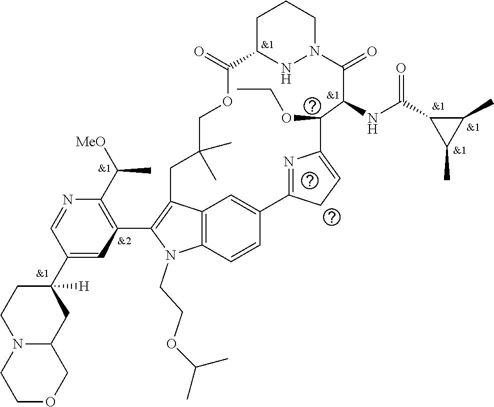 |
| A862 | 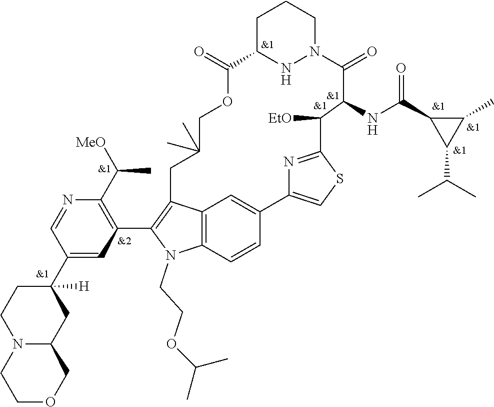 |
| A863 | 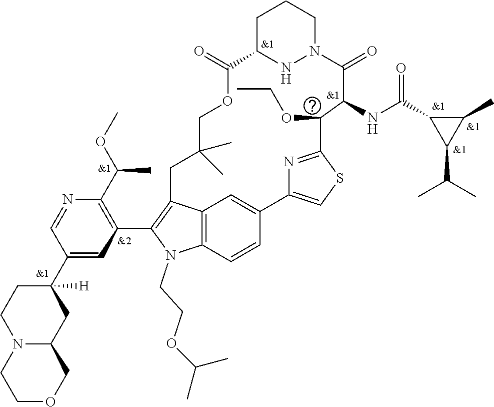 |
| A864 | 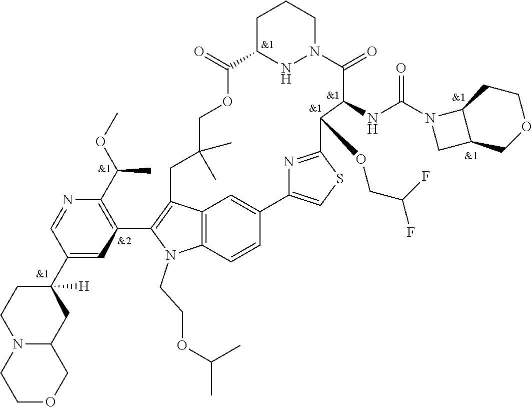 |
| A865 | 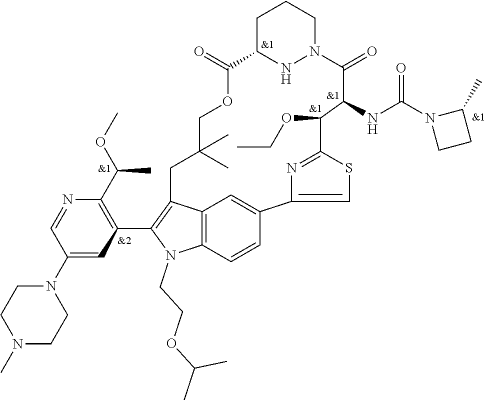 |
| A866 | 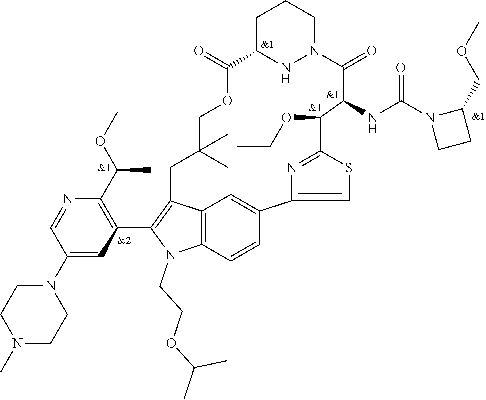 |
| A867 | 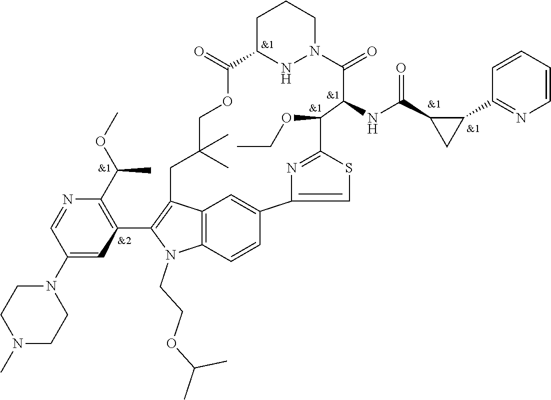 |
| A868 | 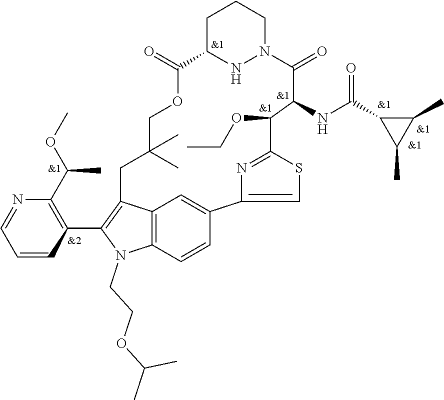 |

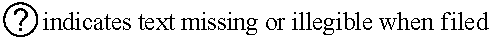

In some embodiments, a compound of the present invention is:

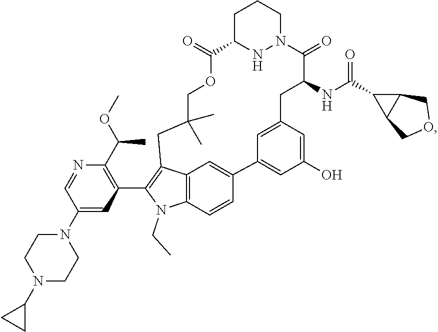

or a pharmaceutically acceptable salt thereof.

In some embodiments, a compound of the present invention is selected from Table 2, or a pharmaceutically acceptable salt or stereoisomer thereof. In some embodiments, a compound of the present invention is selected from Table 2, or a pharmaceutically acceptable salt or atropisomer thereof.

### Table 2. Certain Compounds of the Present Invention

TABLE 2
Certain Compounds of the Present Invention

| Ex# | Structure |
|---|---|
| A869 | 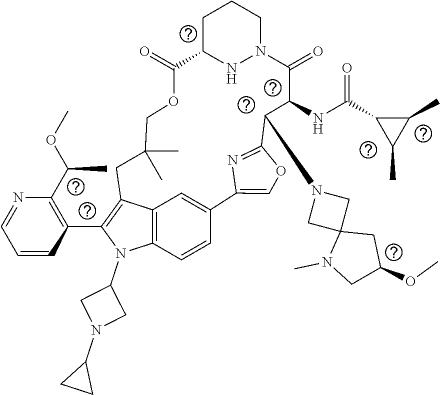 |
| A870 | 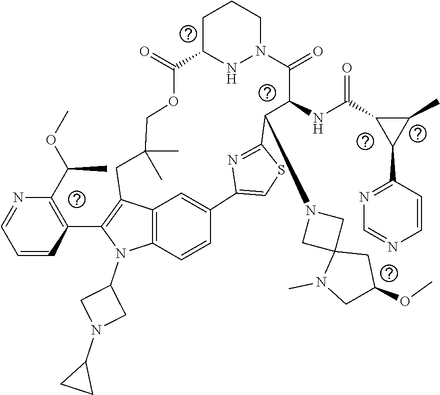 |
| A871 | 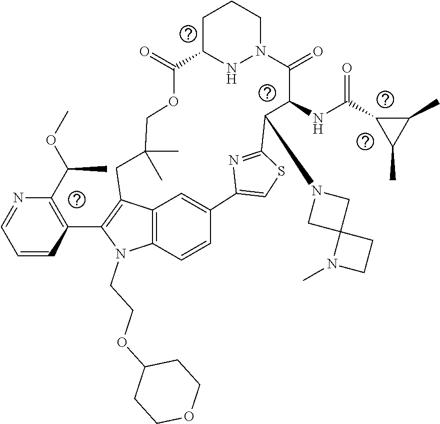 |
| A872 | 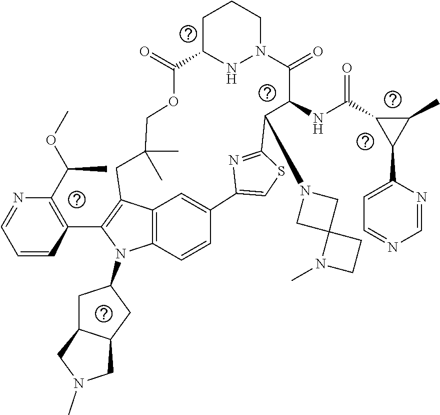 |
| A873 | 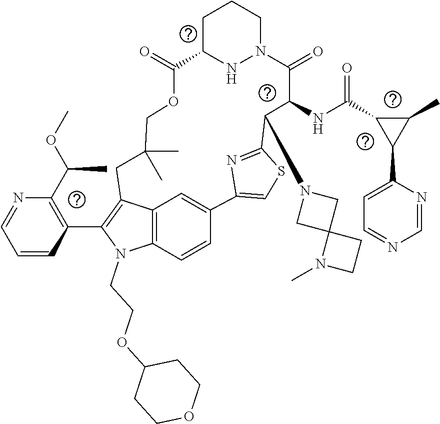 |
| A874 | 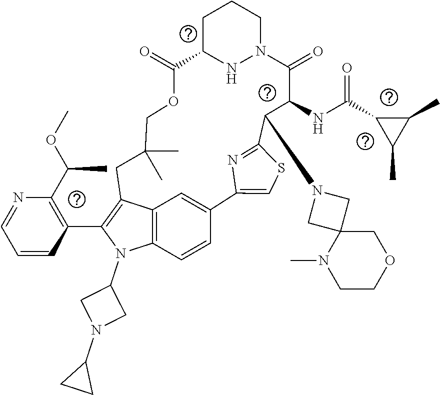 |
| A875 | 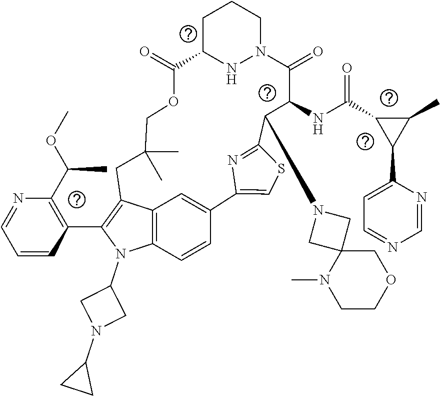 |
| A876 | 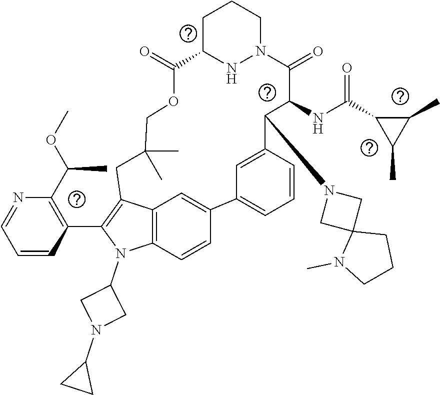 |
| A877 | 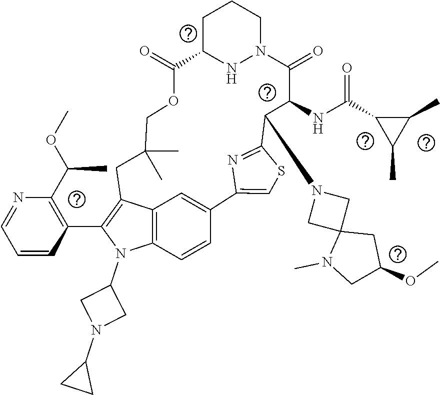 |
| A878 | 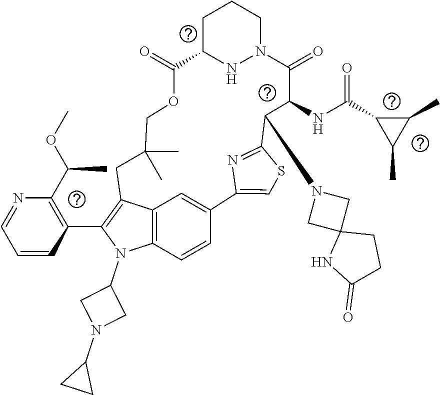 |
| A879 | 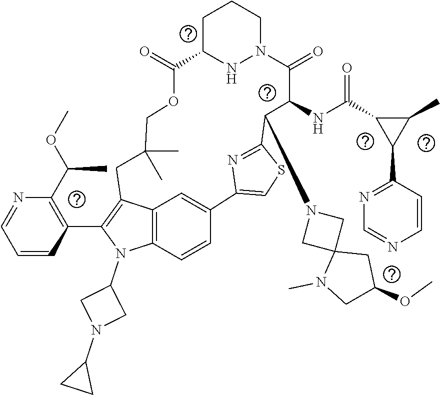 |
| A880 | 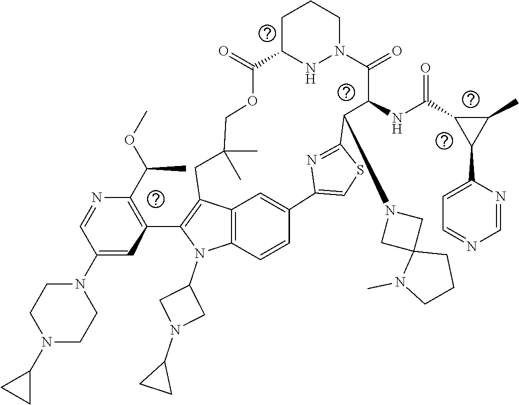 |
| A881 | 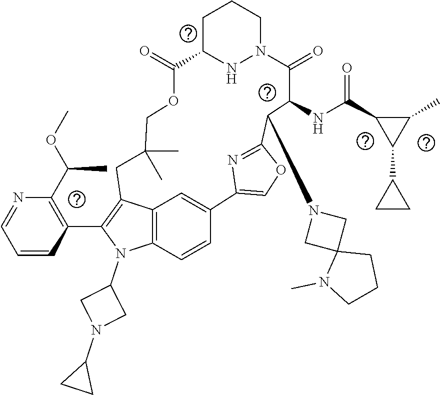 |
| A882 | 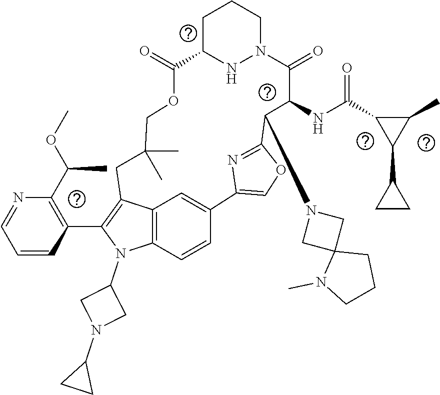 |
| A883 | 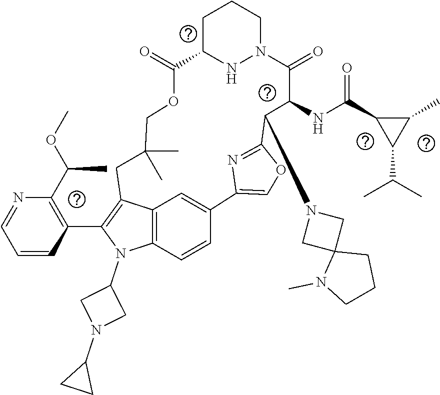 |
| A884 | 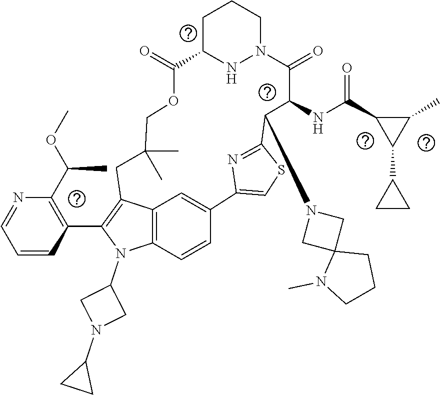 |
| A885 | 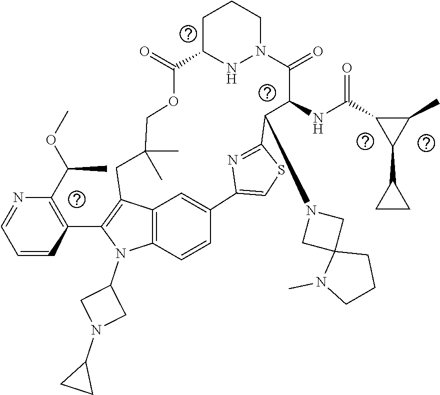 |
| A886 | 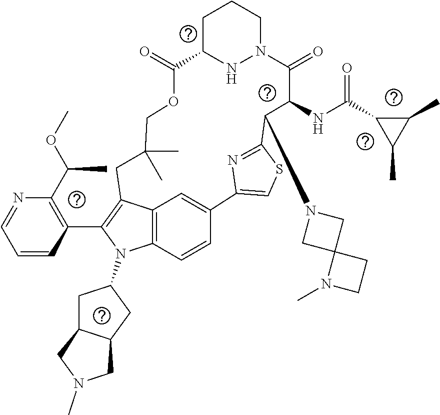 |
| A887 | 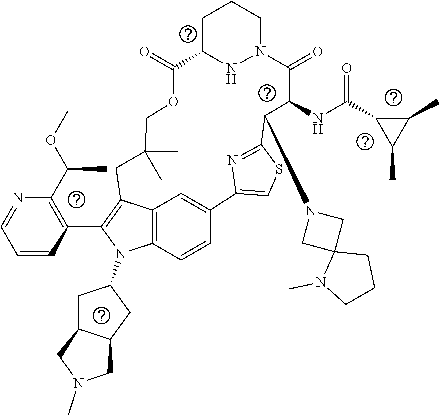 |
| A888 | 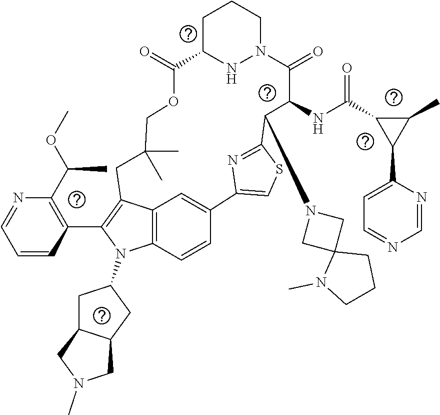 |
| A889 | 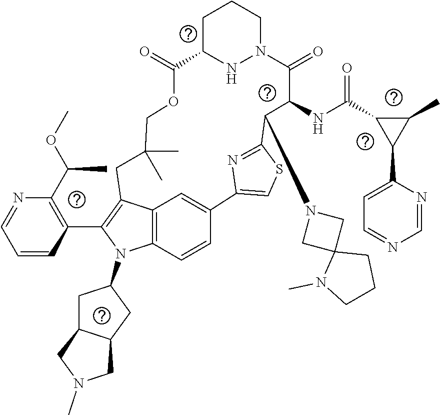 |
| A890 | 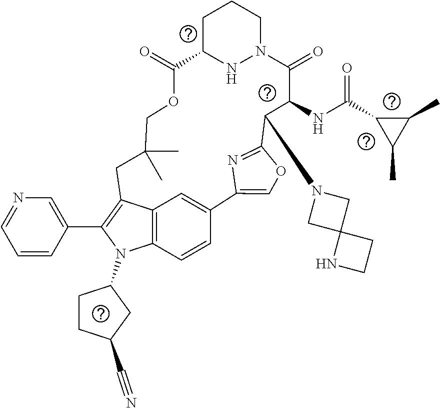 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@H]3CC[C@H](C#N)C3)c3ccc(cc23)-c2coc(n2)[C@H]1N1CC2(CCN2)C1] |
| A891 | 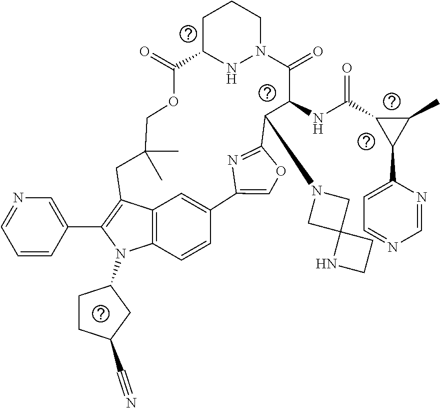 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3coc(n3)[C@H]2N2CC3(CCN3)C2)[C@@H]1c1ccncn1] |
| A892 | 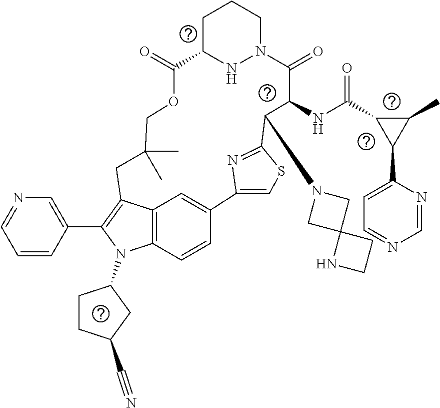 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3csc(n3)[C@H]2N2CC3(CCN3)C2)[C@@H]1c1ccncn1] |
| A893 | 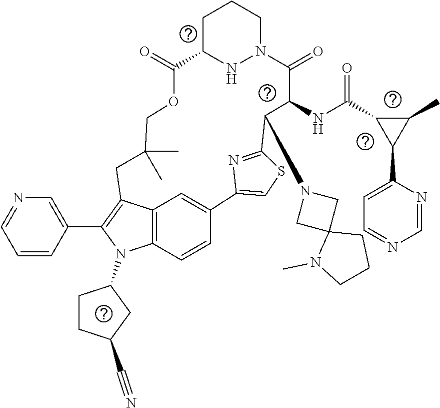 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3csc(n3)[C@H]2N2CC3(CCCN3C)C2)[C@@H]1c1ccncn1] |
| A894 | 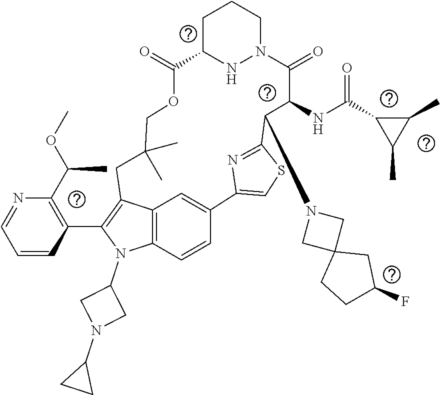 |
| A895 | 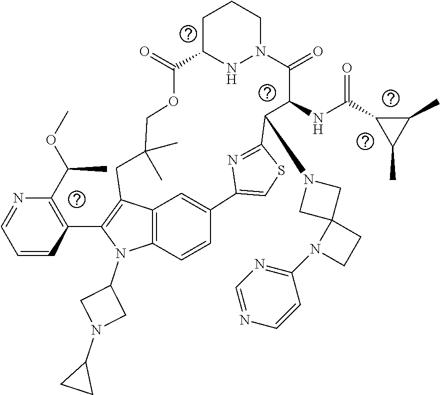 |
| A896 | 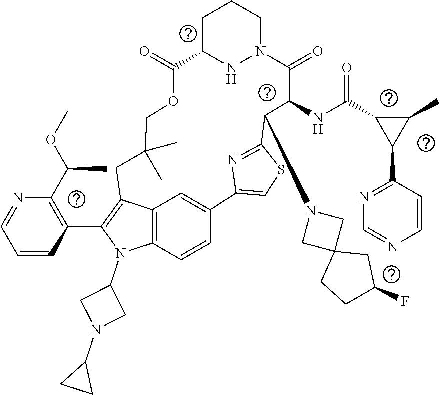 |
| A897 | 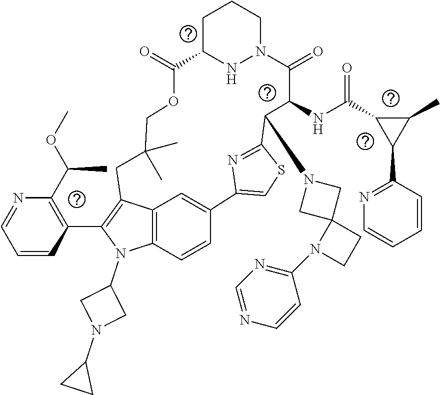 |
| A898 | 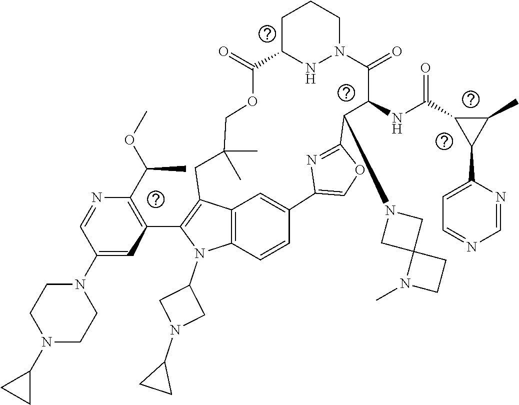 |
| A899 | 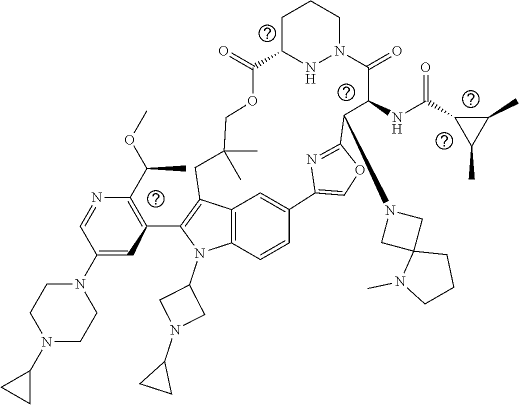 |
| A900 | 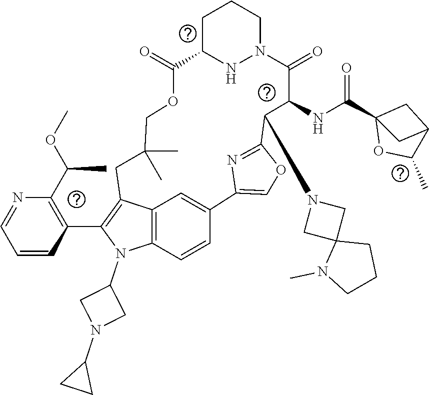 |
| A901 | 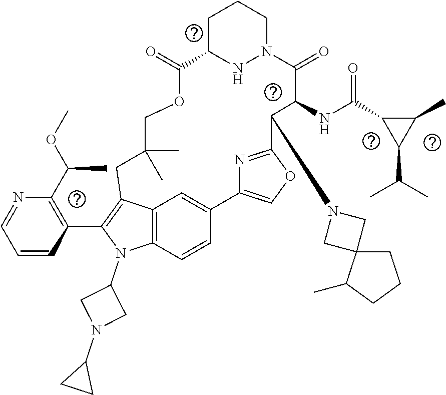 |
| A902 | 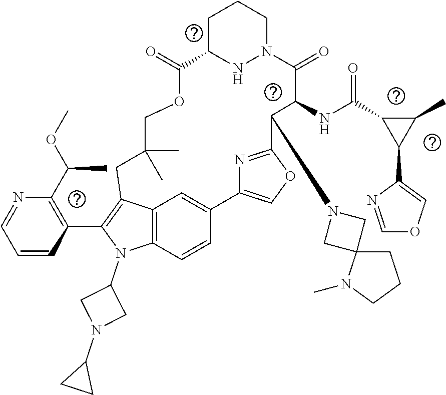 |
| A903 | 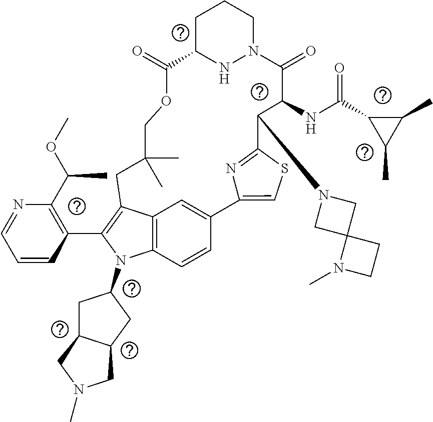 |
| A904 | 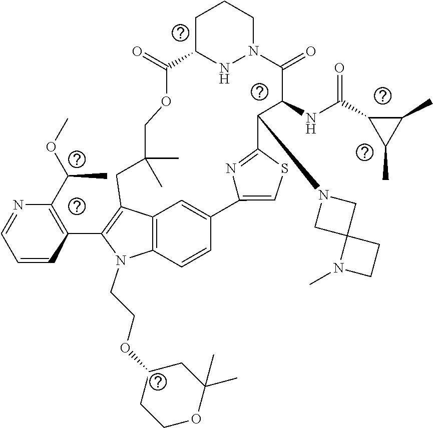 |
| A905 | 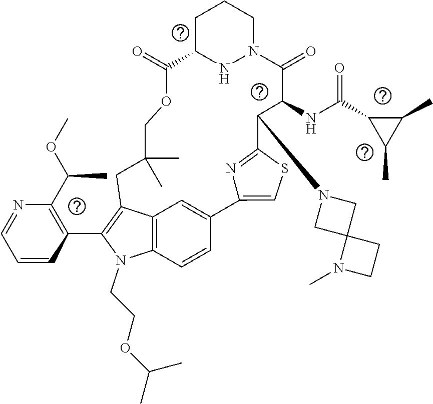 |
| A906 | 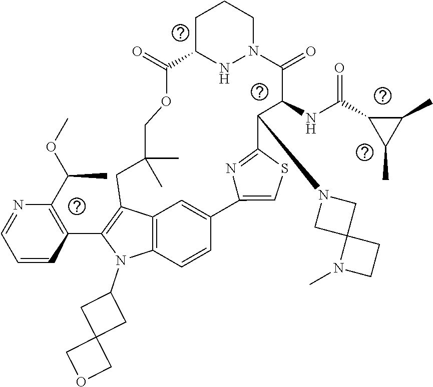 |
| A907 | 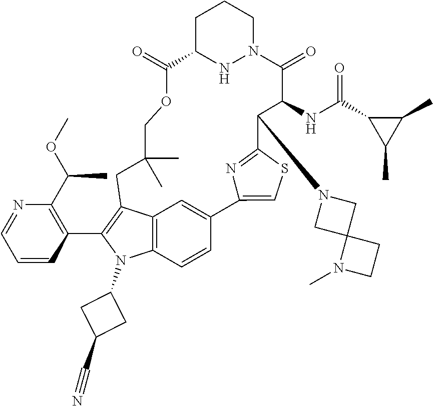 |
| A908 | 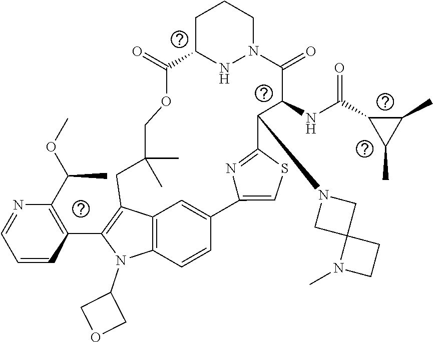 |
| A909 | 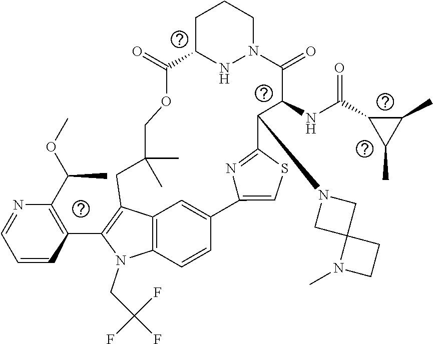 |
| A910 | 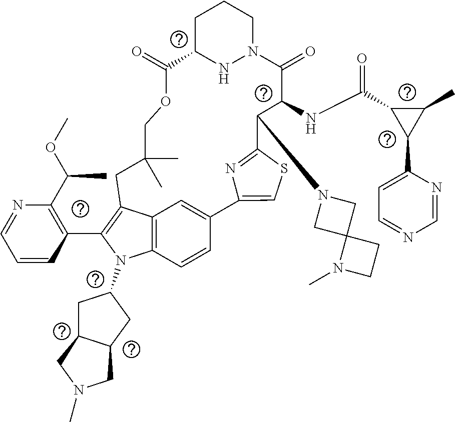 |
| A911 | 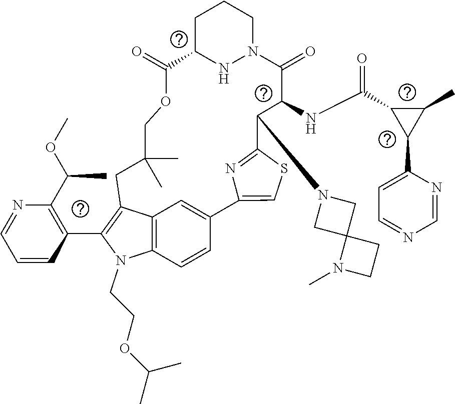 |
| A912 | 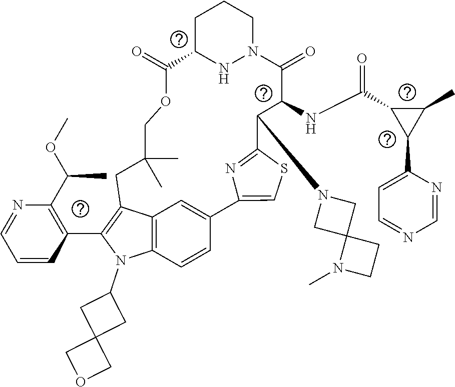 |
| A913 | 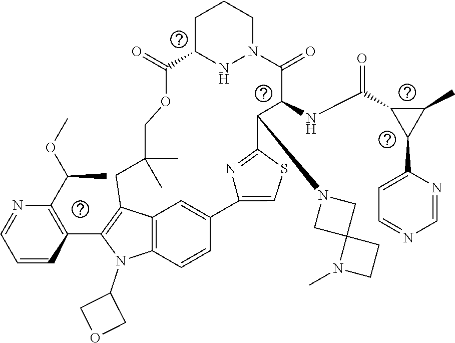 |
| A914 | 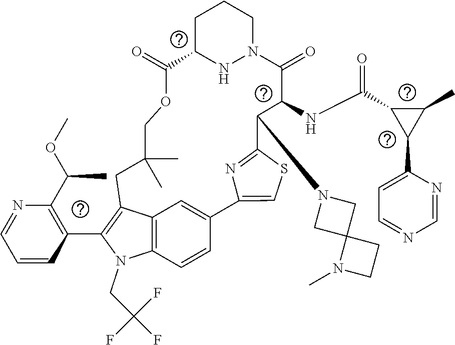 |
| A915 | 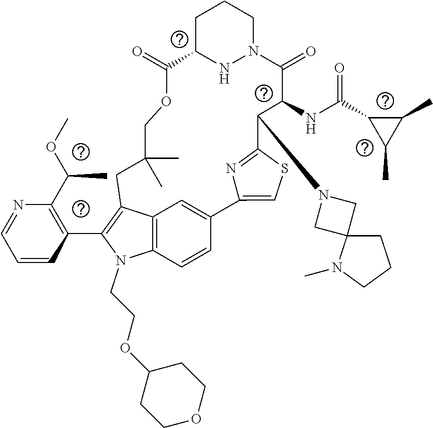 |
| A916 | 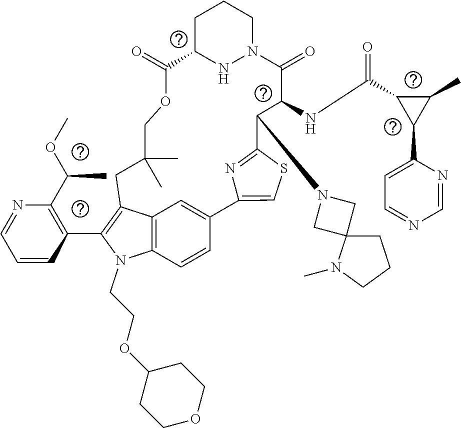 |
| A917 | 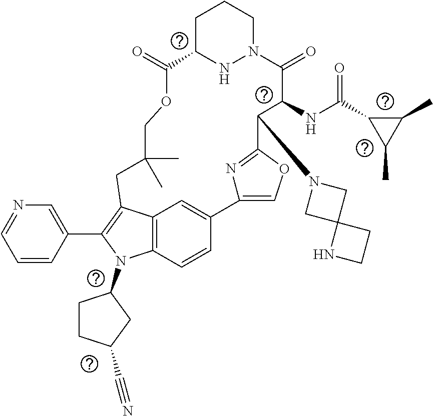 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@@H]3CC[C@@H](C#N)C3)c3ccc(cc23)-c2coc(n2)[C@H]1N1CC2(CCN2)C1] |
| A918 | 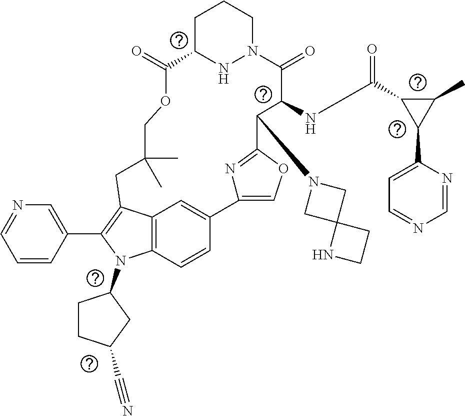 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3coc(n3)[C@H]2N2CC3(CCN3)C2)[C@@H]1c1ccncn1] |
| A919 | 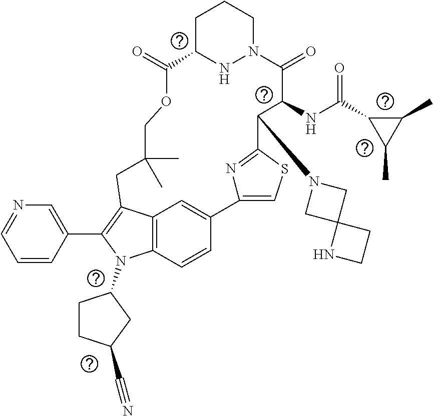 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@H]3CC[C@H](C#N)C3)c3ccc(cc23)-c2csc(n2)[C@H]1N1CC2(CCN2)C1] |
| A920 | 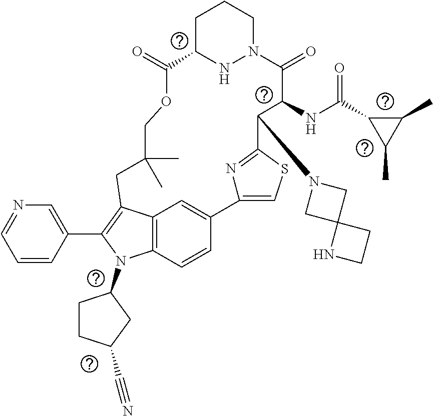 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@@H]3CC[C@@H](C#N)C3)c3ccc(cc23)-c2csc(n2)[C@H]1N1CC2(CCN2)C1] |
| A921 | 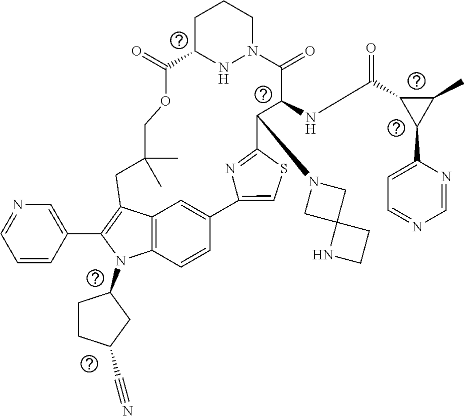 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3csc(n3)[C@H]2N2CC3(CCN3)C2)[C@@H]1c1ccncn1] |
| A922 | 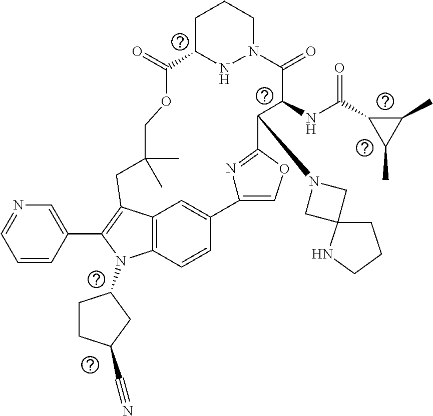 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@H]3CC[C@H](C#N)C3)c3ccc(cc23)-c2coc(n2)[C@H]1N1CC2(CCCN2)C1] |
| A923 | 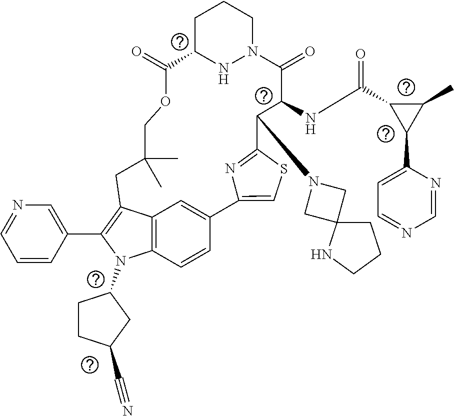 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3csc(n3)[C@H]2N2CC3(CCCN3)C2)[C@@H]1c1ccncn1] |
| A924 | 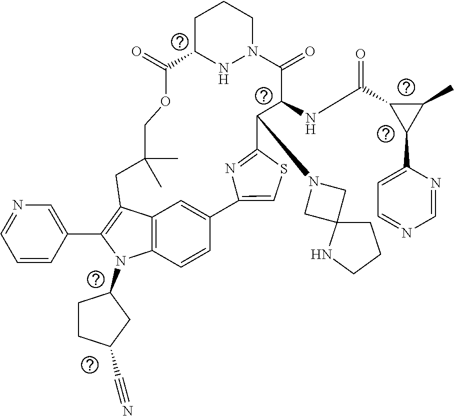 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3csc(n3)[C@H]2N2CC3(CCCN3)C2)[C@@H]1c1ccncn1] |
| A925 | 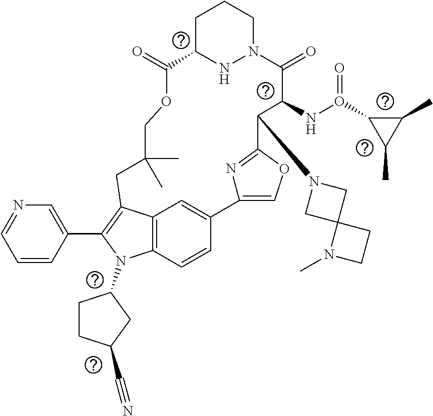 |
| A926 | 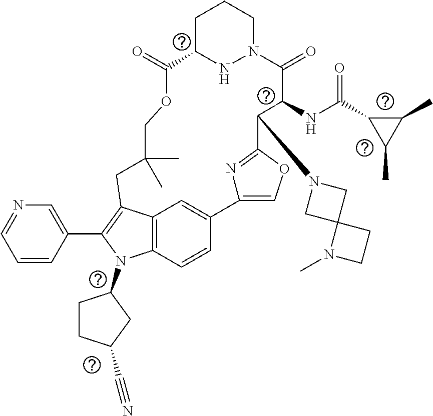 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@@H]3CC[C@@H](C#N)C3)c3ccc(cc23)-c2coc(n2)[C@H]1N1CC2(CCN2C)C1] |
| A927 | 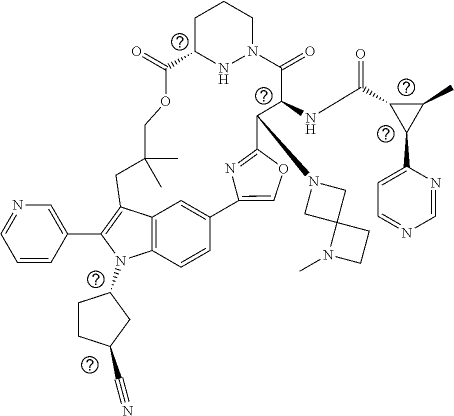 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3coc(n3)[C@H]2N2CC3(CCN3C)C2)[C@@H]1c1ccncn1] |
| A928 | 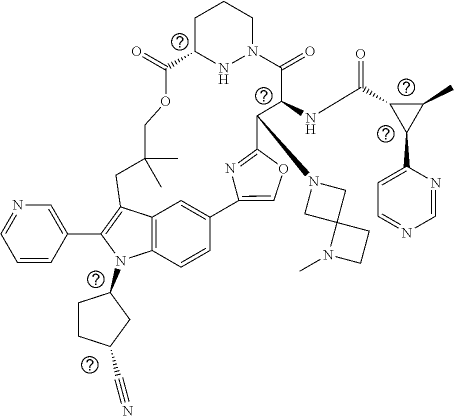 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3coc(n3)[C@H]2N2CC3(CCN3C)C2)[C@@H]1c1ccncn1] |
| A929 | 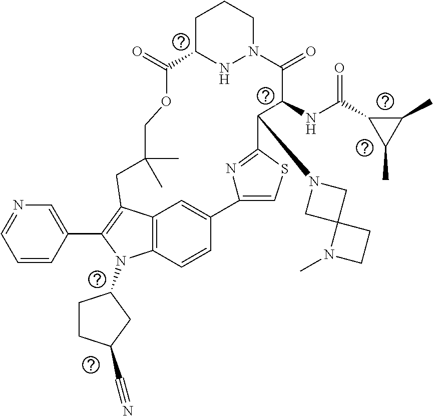 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@H]3CC[C@H](C#N)C3)c3ccc(cc23)-c2csc(n2)[C@H]1N1CC2(CCN2C)C1] |
| A930 | 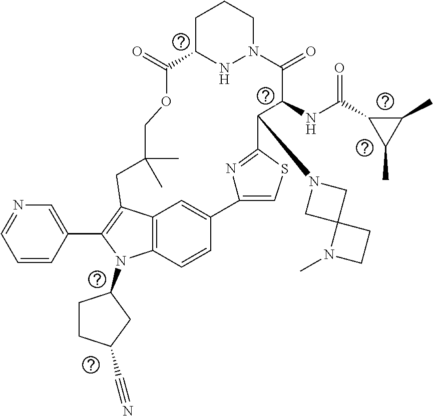 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@@H]3CC[C@@H](C#N)C3)c3ccc(cc23)-c2csc(n2)[C@H]1N1CC2(CCN2C)C1] |
| A931 | 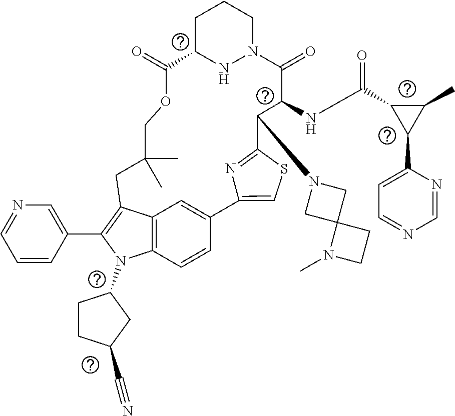 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3csc(n3)[C@H]2N2CC3(CCN3C)C2)[C@@H]1c1ccncn1] |
| A932 | 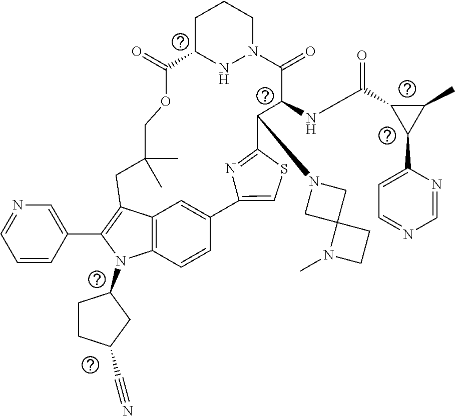 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3csc(n3)[C@H]2N2CC3(CCN3C)C2)[C@@H]1c1ccncn1] |
| A933 | 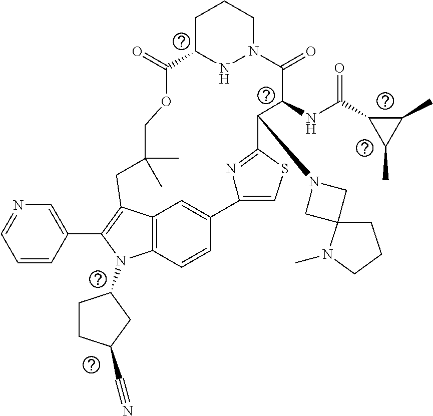 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@H]3CC[C@H](C#N)C3)c3ccc(cc23)-c2csc(n2)[C@H]1N1CC2(CCCN2C)C1] |
| A934 | 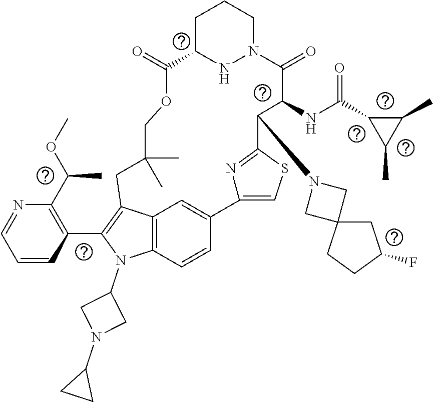 |
| A935 | 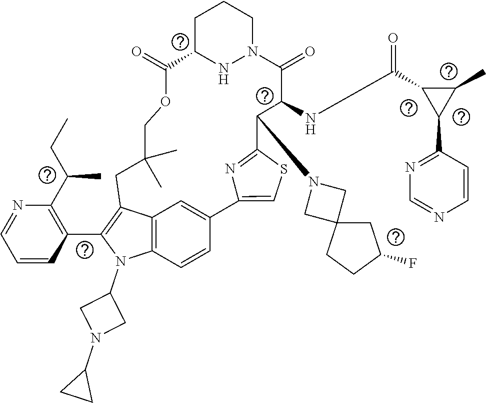 |
| A936 | 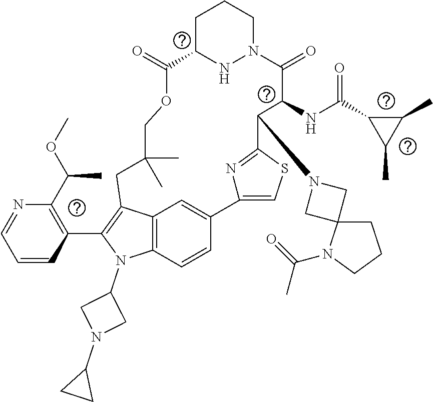 |
| A937 | 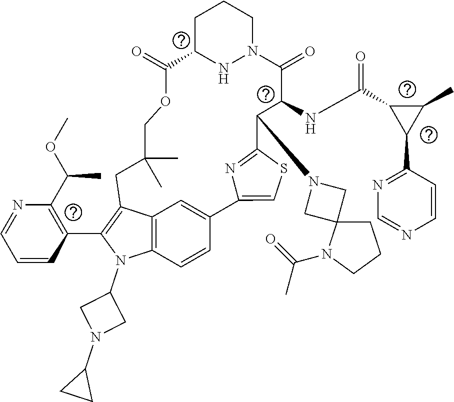 |
| A938 | 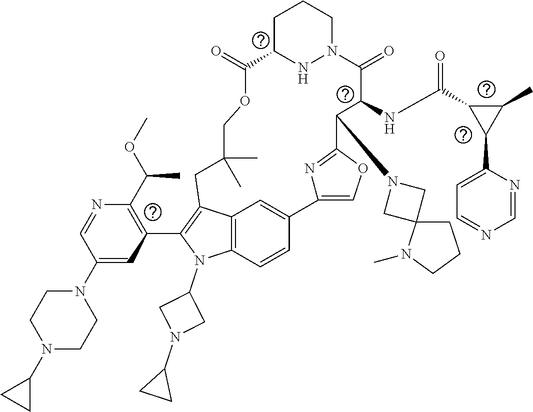 |
| A939 | 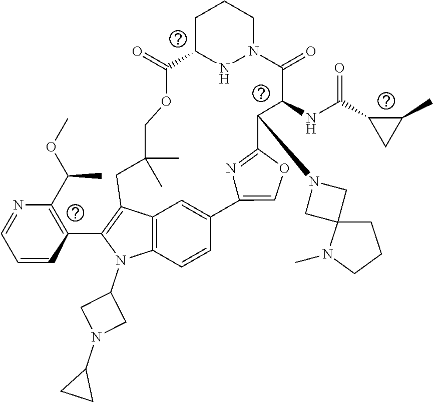 |
| A940 | 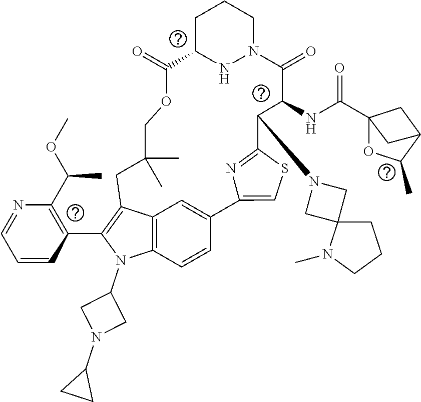 |
| A941 | 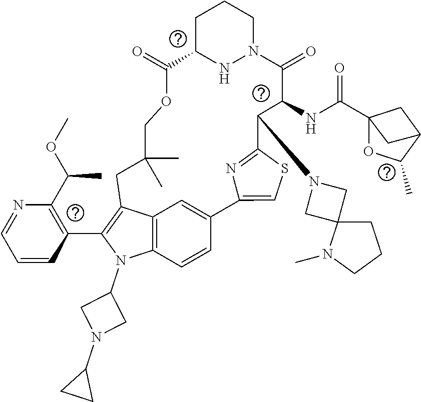 |
| A942 | 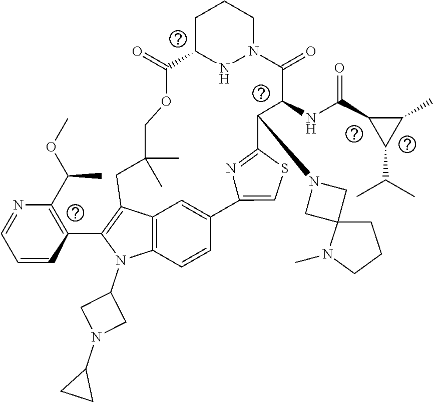 |
| A943 | 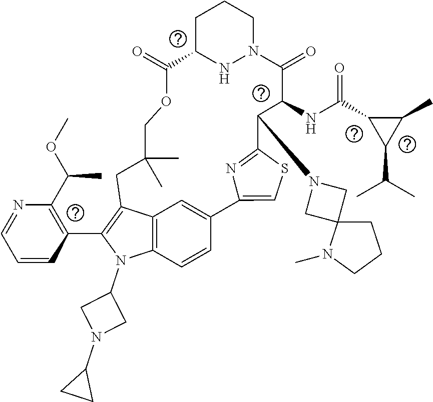 |
| A944 | 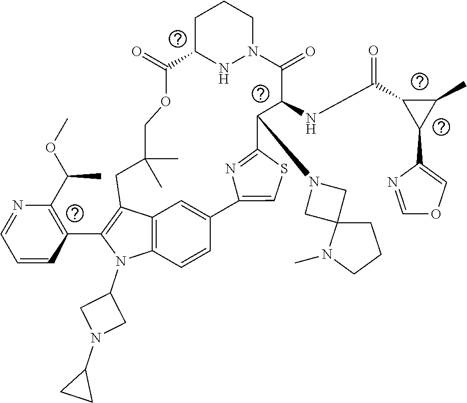 |
| A945 | 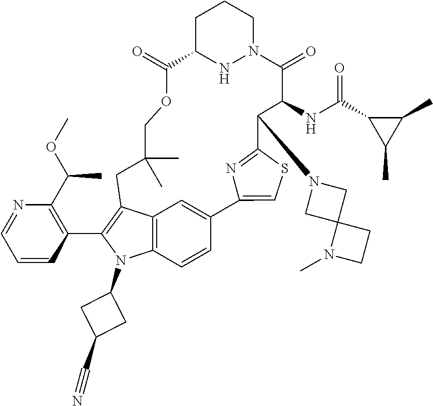 |
| A946 | 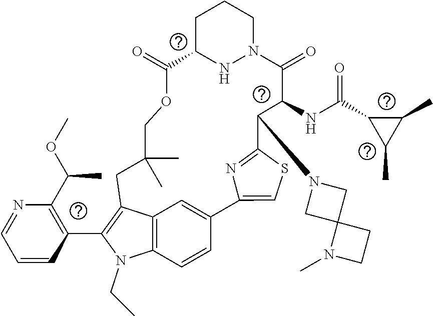 |
| A947 | 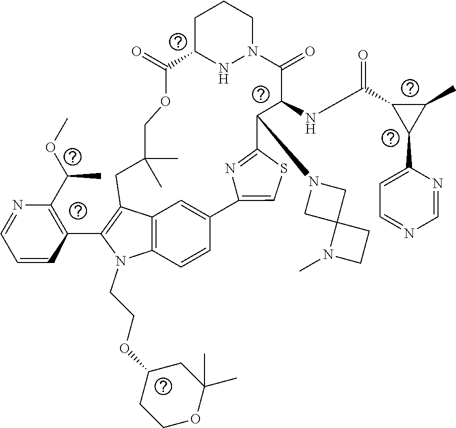 |
| A948 | 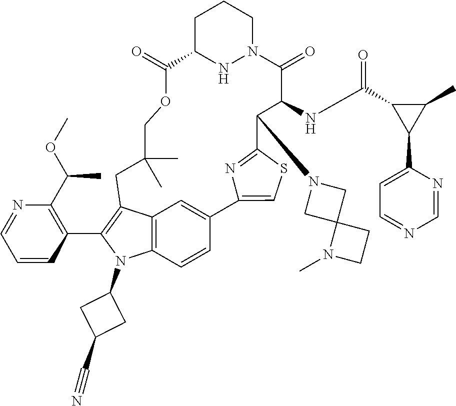 |
| A949 | 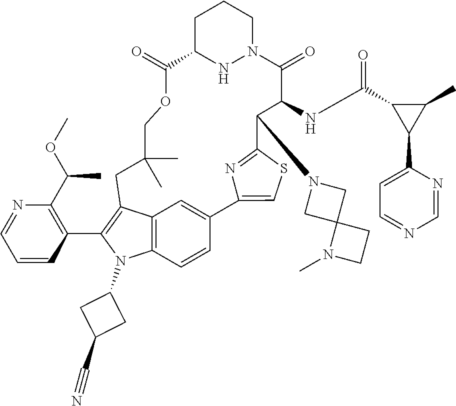 |
| A950 | 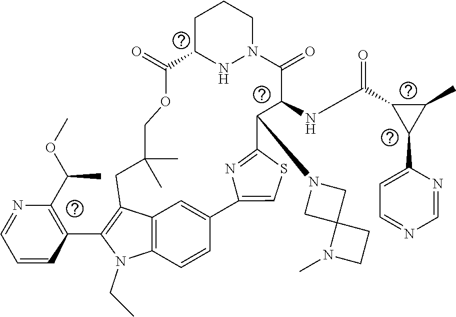 |
| A951 | 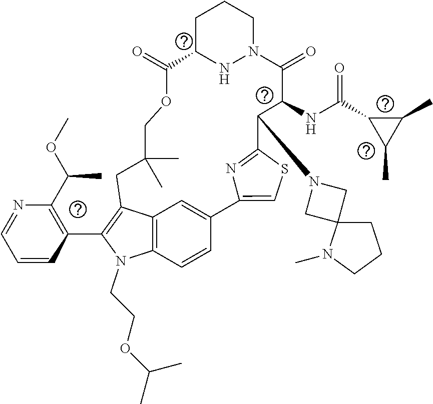 |
| A952 | 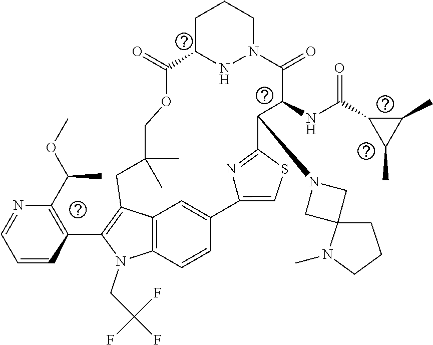 |
| A953 | 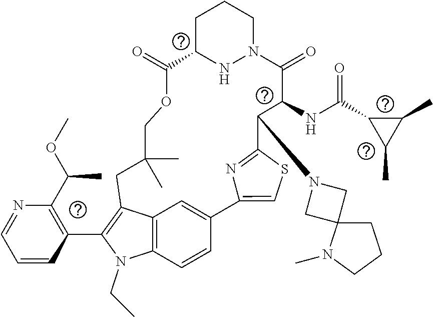 |
| A954 | 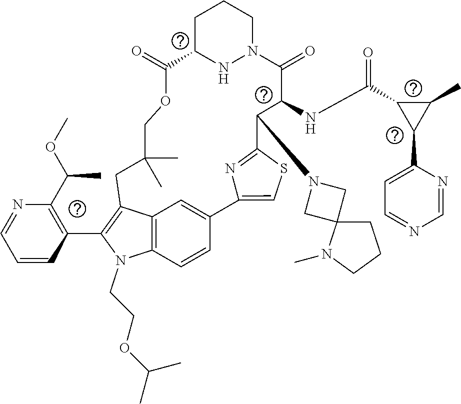 |
| A955 | 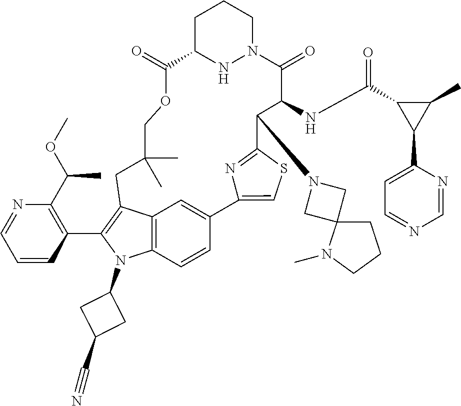 |
| A956 | 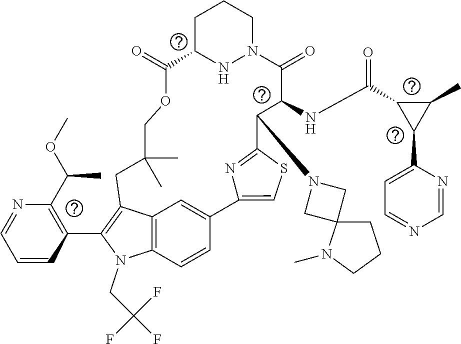 |
| A957 | 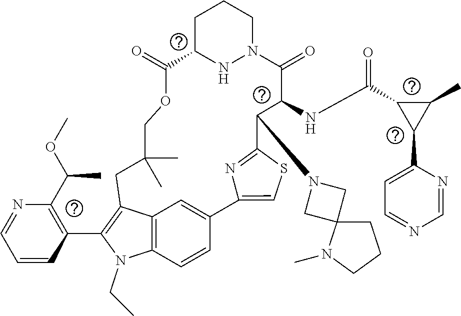 |
| A958 | 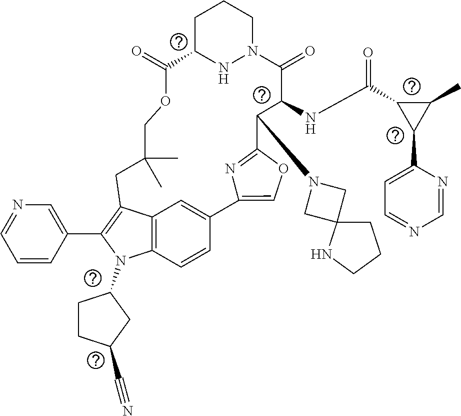 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3coc(n3)[C@H]2N2CC3(CCCN3)C2)[C@@H]1c1ccncn1] |
| A959 | 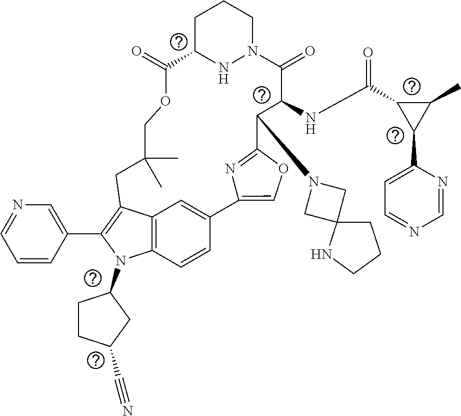 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3coc(n3)[C@H]2N2CC3(CCCN3)C2)[C@@H]1c1ccncn1] |
| A960 | 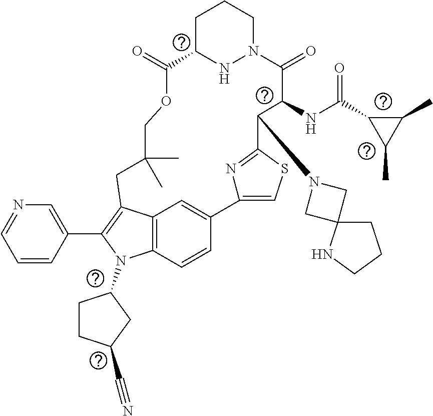 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@H]3CC[C@H](C#N)C3)c3ccc(cc23)-c2csc(n2)[C@H]1N1CC2(CCCN2)C1] |
| A961 | 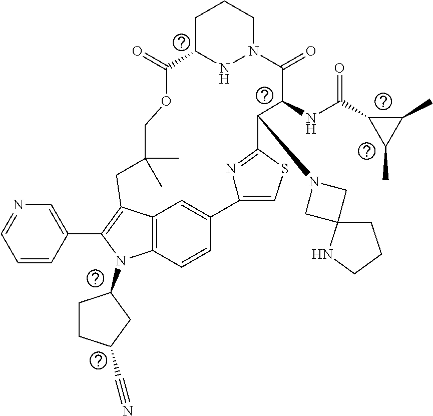 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@@H]3CC[C@@H](C#N)C3)c3ccc(cc23)-c2csc(n2)[C@H]1N1CC2(CCCN2)C1] |
| A962 | 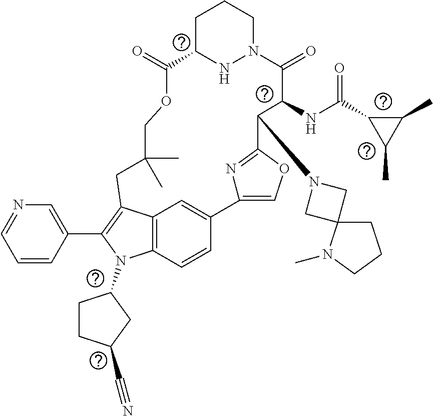 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@H]3CC[C@H](C#N)C3)c3ccc(cc23)-c2coc(n2)[C@H]1N1CC2(CCCN2C)C1] |
| A963 | 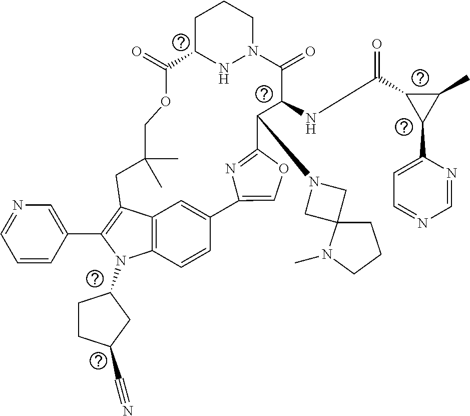 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3coc(n3)[C@H]2N2CC3(CCCN3C)C2)[C@@H]1c1ccncn1] |
| A964 | 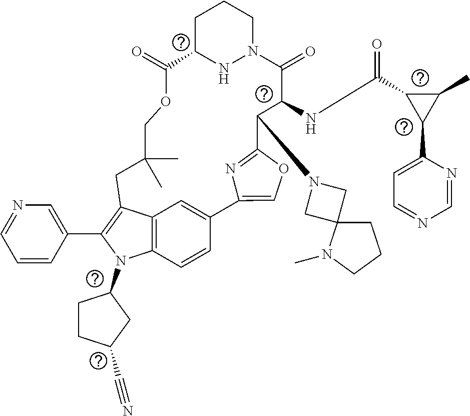 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3coc(n3)[C@H]2N2CC3(CCCN3C)C2)[C@@H]1c1ccncn1] |
| A965 | 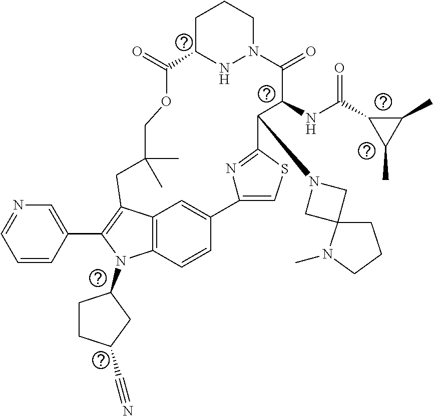 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@@H]3CC[C@@H](C#N)C3)c3ccc(cc23)-c2csc(n2)[C@H]1N1CC2(CCCN2C)C1] |
| A966 | 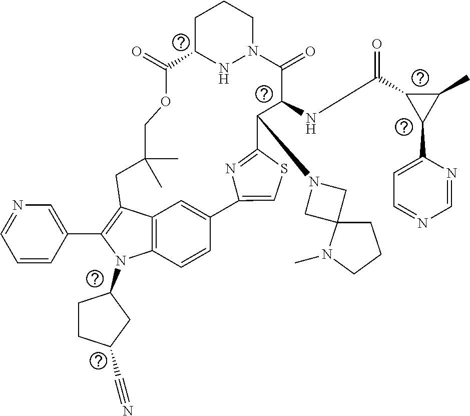 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3csc(n3)[C@H]2N2CC3(CCCN3C)C2)[C@@H]1c1ccncn1] |
| A967 | 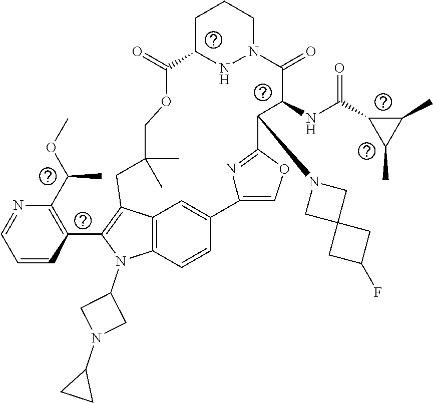 |
| A968 | 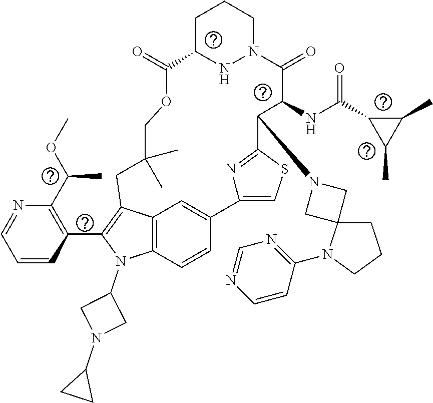 |
| A969 | 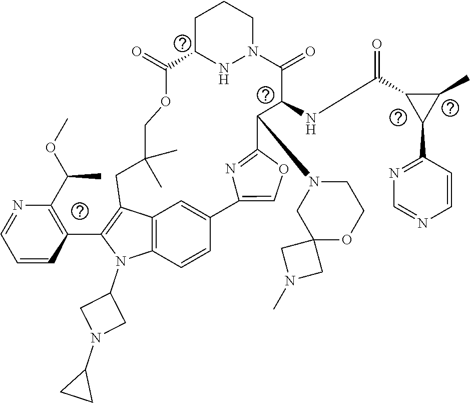 |
| A970 | 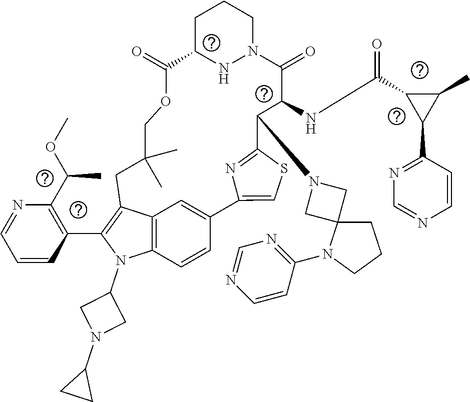 |
| A971 | 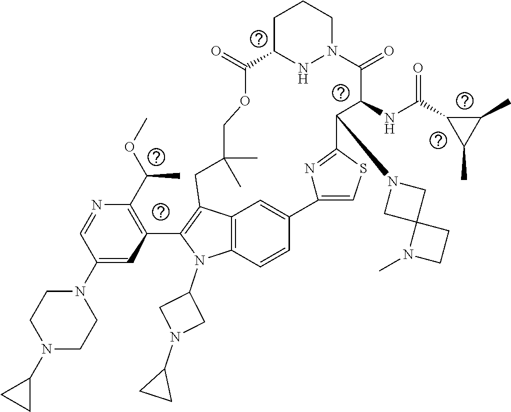 |
| A972 | 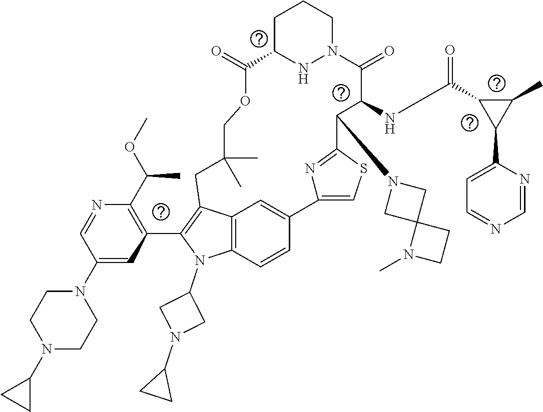 |
| A973 | 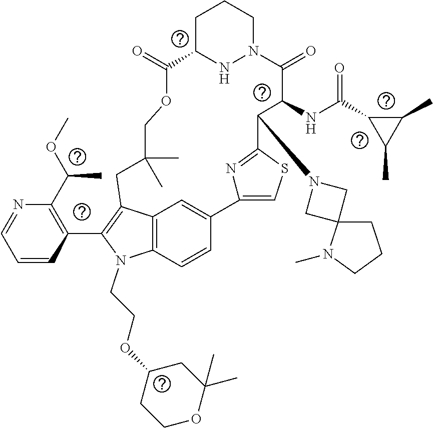 |
| A974 | 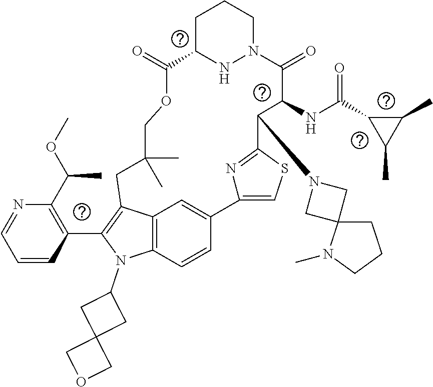 |
| A975 | 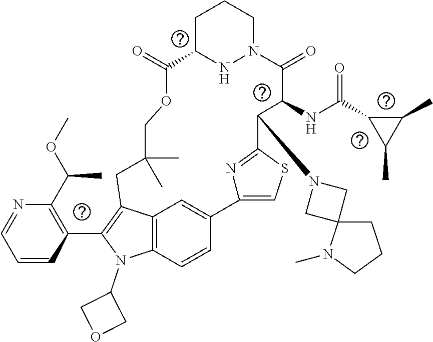 |
| A976 | 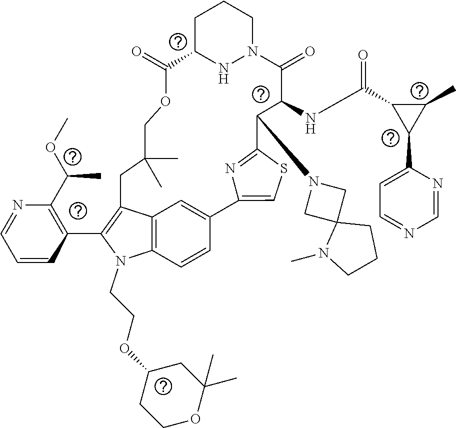 |
| A977 | 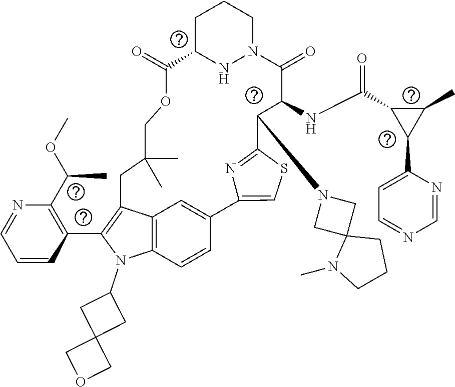 |
| A978 | 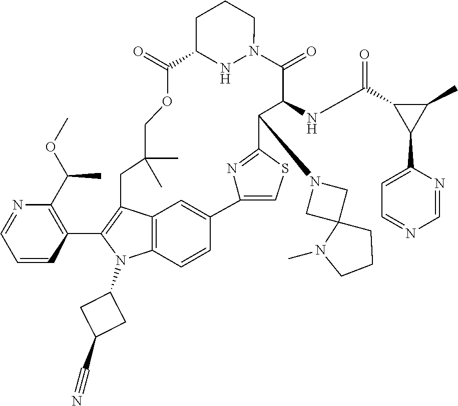 |
| A979 | 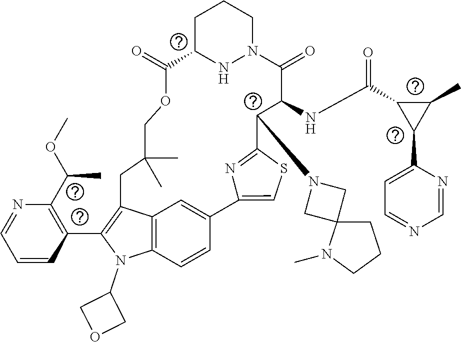 |
| A980 | 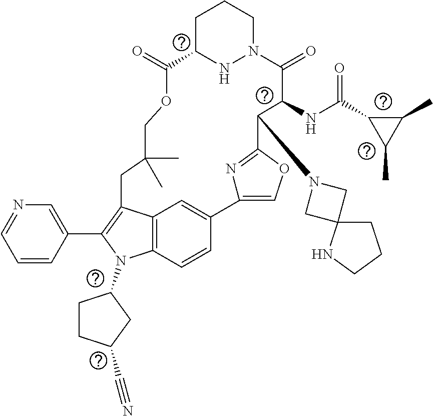 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@H]3CC[C@@H](C#N)C3)c3ccc(cc23)-c2coc(n2)[C@H]1N1CC2(CCCN2)C1] |
| A981 | 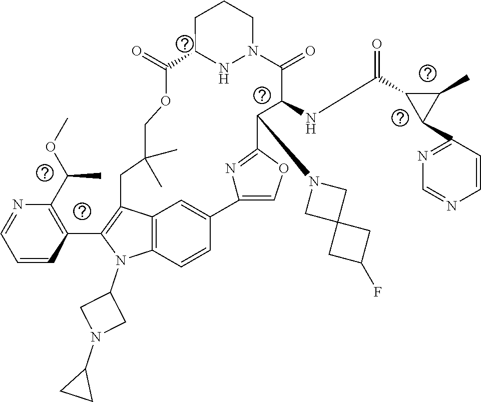 |
| A982 | 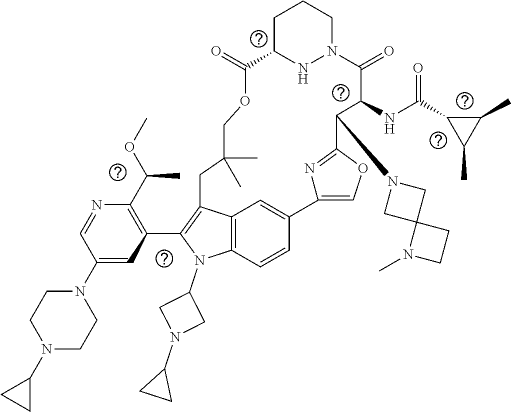 |
| A983 | 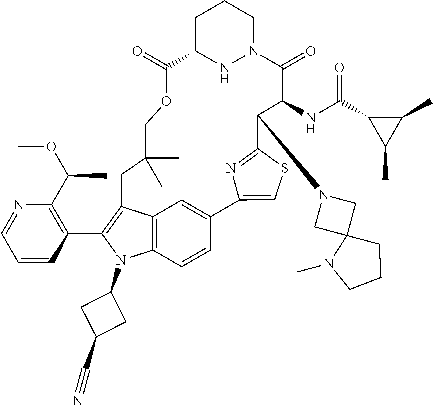 |
| A984 | 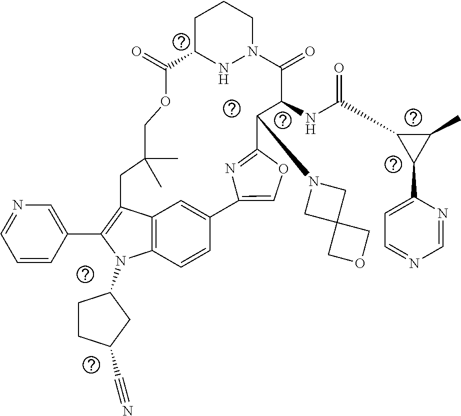 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@@H](C#N)C4)c4ccc(cc34)-c3coc(n3)[C@H]2N2CC3(COC3)C2)[C@@H]1c1ccncn1] |
| A985 | 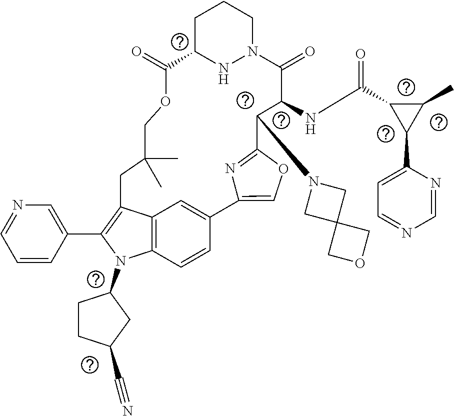 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@H](C#N)C4)c4ccc(cc34)-c3coc(n3)[C@H]2N2CC3(COC3)C2)[C@@H]1c1ccncn1] |
| A986 | 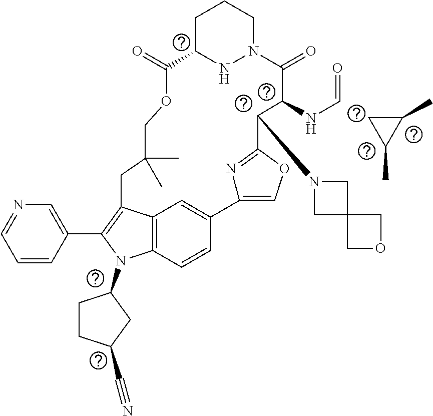 |
| A987 | 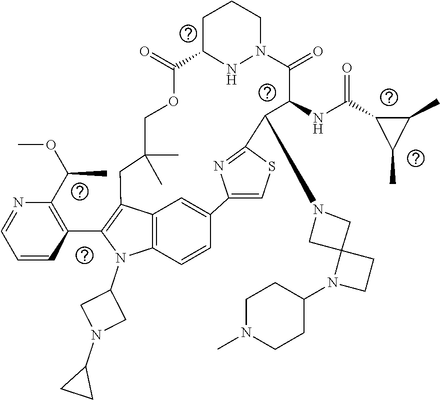 |
| A988 | 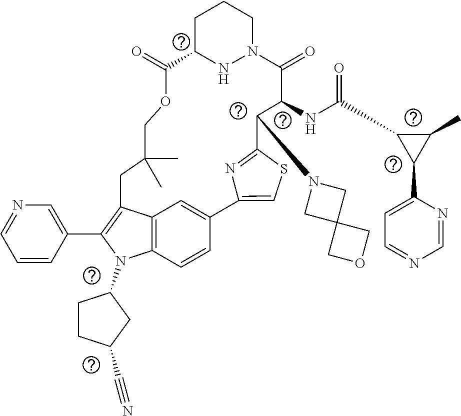 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@@H](C#N)C4)c4ccc(cc34)-c3csc(n3)[C@H]2N2CC3(COC3)C2)[C@@H]1c1ccncn1] |
| A989 | 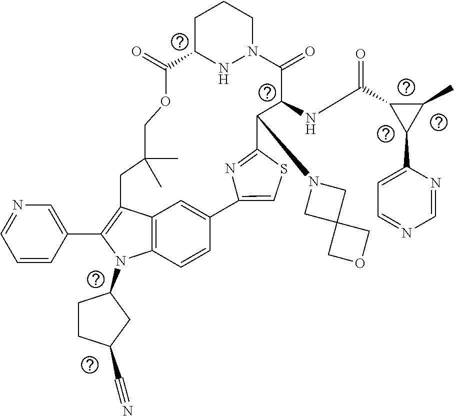 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@H](C#N)C4)c4ccc(cc34)-c3csc(n3)[C@H]2N2CC3(COC3)C2)[C@@H]1c1ccncn1] |
| A990 | 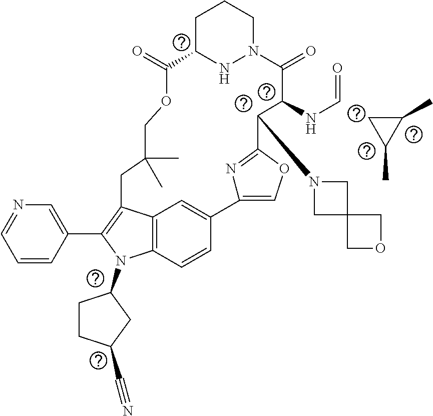 |
| A991 | 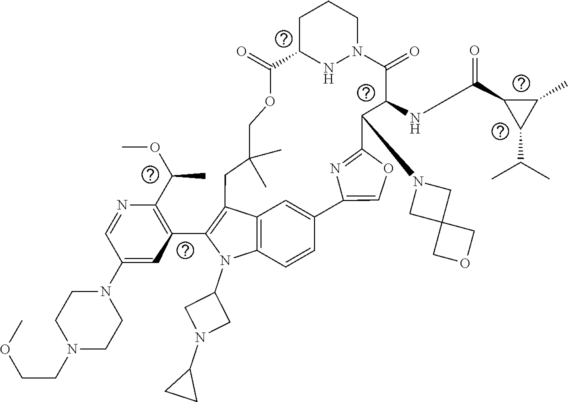 |
| A992 | 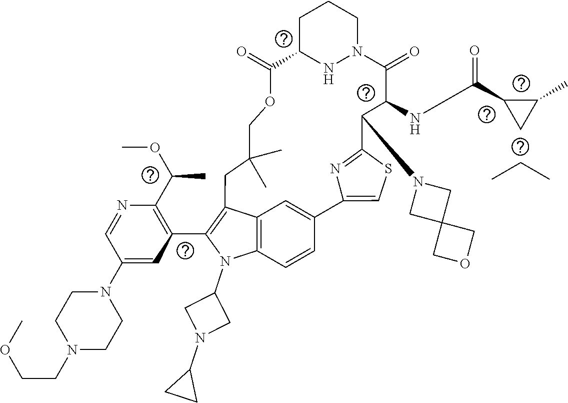 |
| A993 | 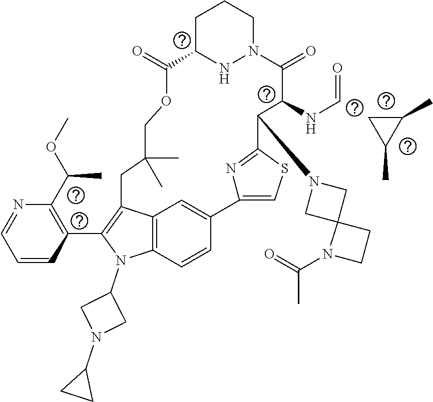 |
| A994 | 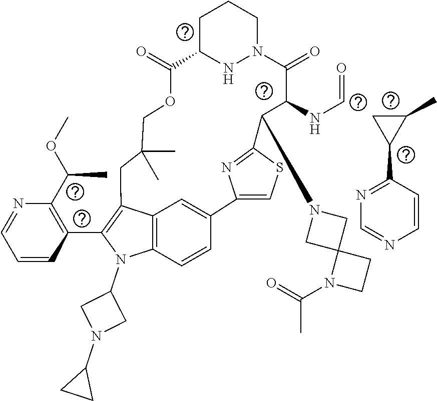 |
| A995 | 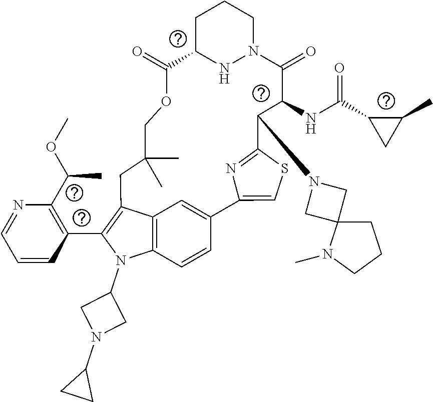 |
| A996 | 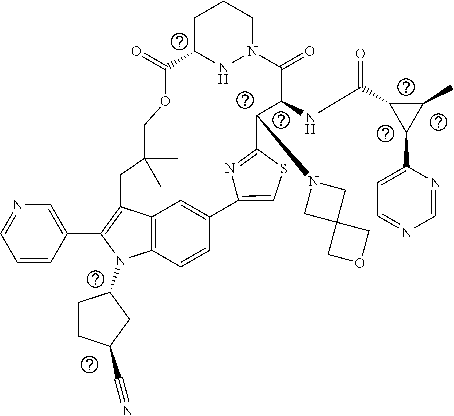 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3csc(n3)[C@H]2N2CC3(COC3)C2)[C@@H]1c1ccncn1] |
| A997 | 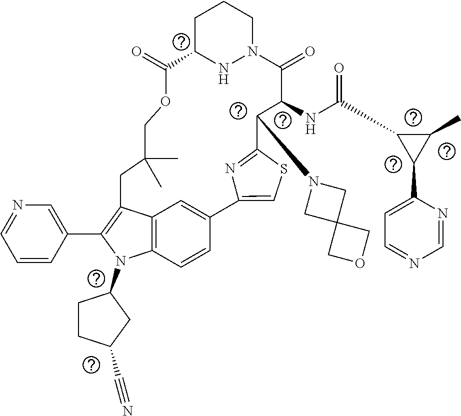 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@@H](C#N)C4)c4ccc(cc34)-c3csc(n3)[C@H]2N2CC3(COC3)C2)[C@@H]1c1ccncn1] |
| A998 | 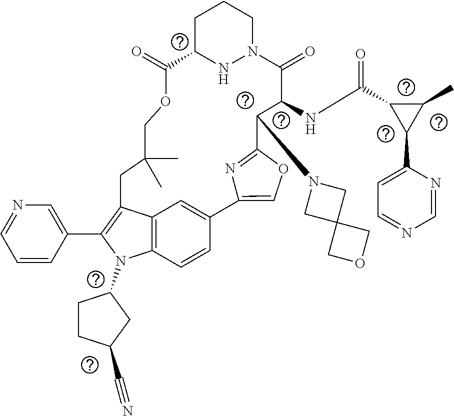 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3coc(n3)[C@H]2N2CC3(COC3)C2)[C@@H]1c1ccncn1] |
| A999 | 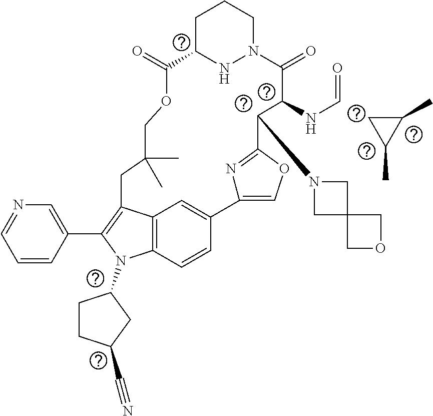 |
| A1000 | 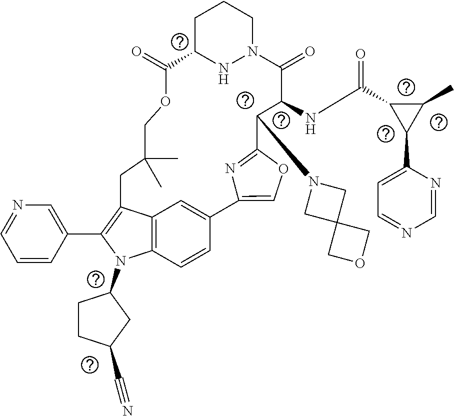 [SMILES: C[C@@H]1[C@@H](C(=O)N[C@@H]2C(=O)N3CCC[C@H](N3)C(=O)OCC(C)(C)Cc3c(-c4cccnc4)n([C@@H]4CC[C@H](C#N)C4)c4ccc(cc34)-c3coc(n3)[C@H]2N2CC3(COC3)C2)[C@@H]1c1ccncn1] |
| A1001 | 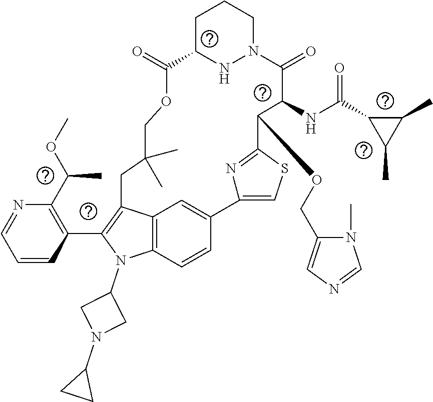 |
| A1002 | 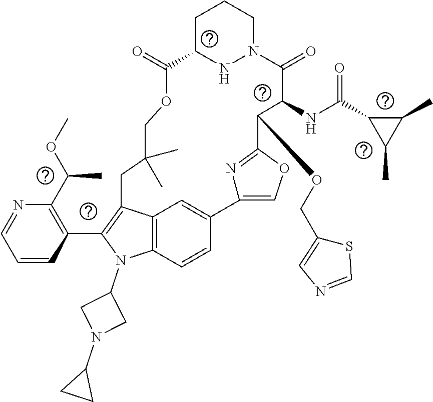 |
| A1003 | 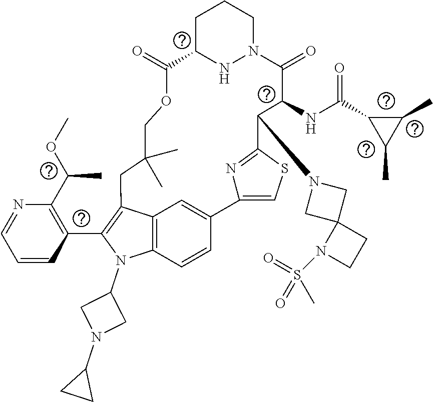 |
| A1004 | 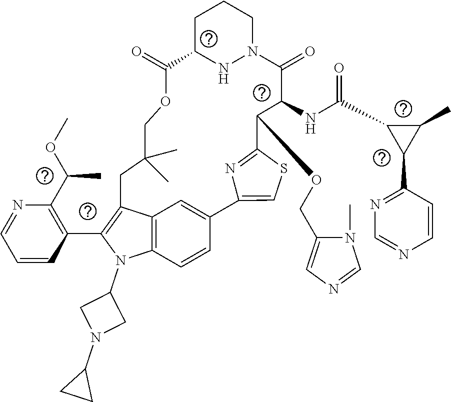 |
| A1005 | 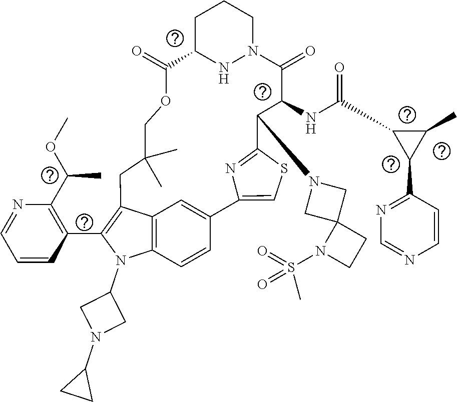 |
| A1006 | 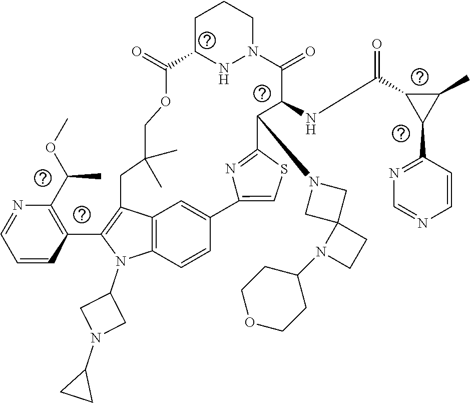 |
| A1007 | 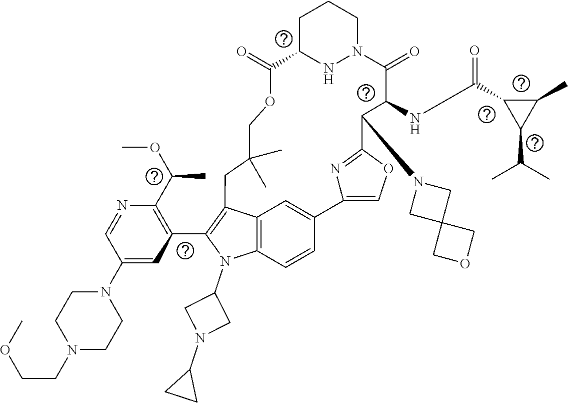 |
| A1008 | 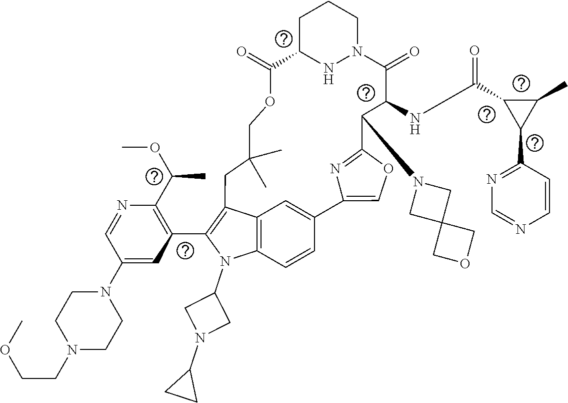 |
| A1009 | 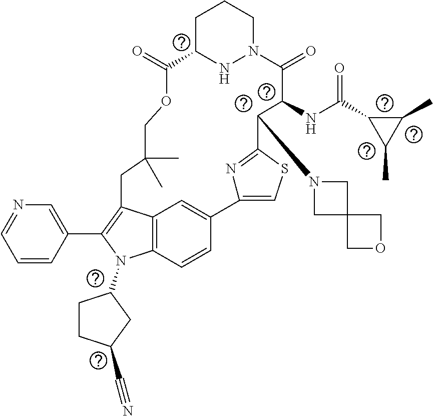 |
| A1010 | 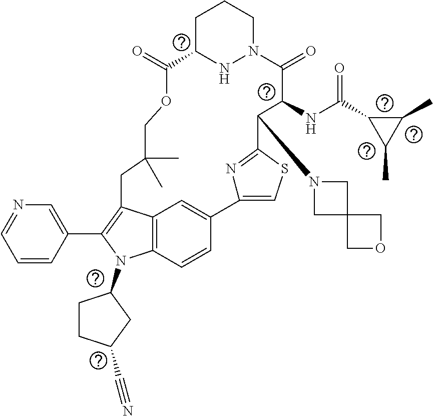 [SMILES: C[C@@H]1[C@H](C)[C@H]1C(=O)N[C@@H]1C(=O)N2CCC[C@H](N2)C(=O)OCC(C)(C)Cc2c(-c3cccnc3)n([C@@H]3CC[C@@H](C#N)C3)c3ccc(cc23)-c2csc(n2)[C@H]1N1CC2(COC2)C1] |
| A1011 | 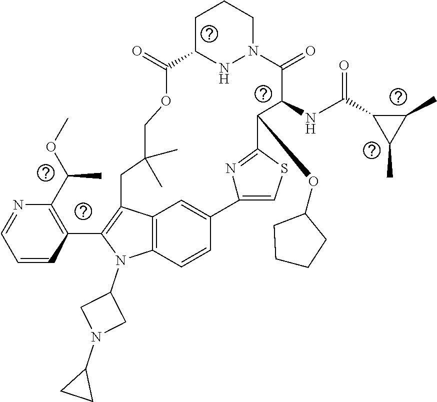 |
| A1012 | 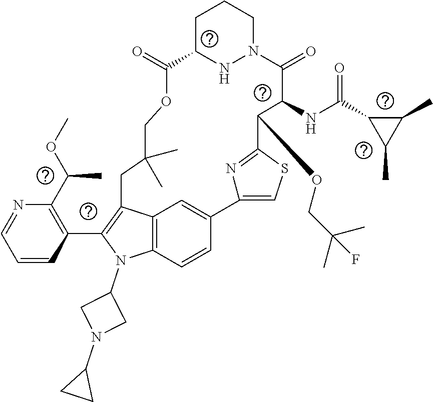 |
| A1013 | 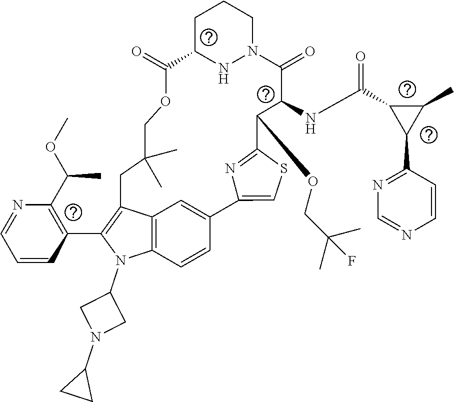 |
| A1014 | 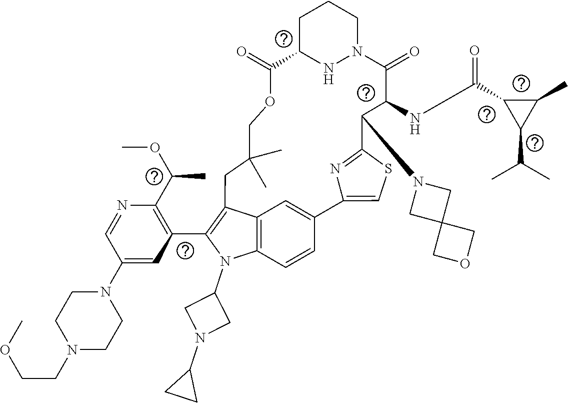 |
| A1015 | 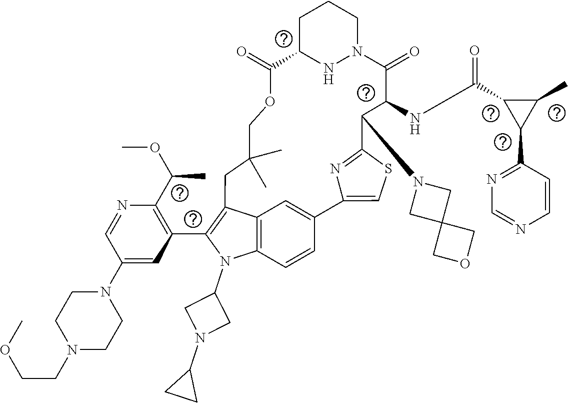 |
| A1016 | 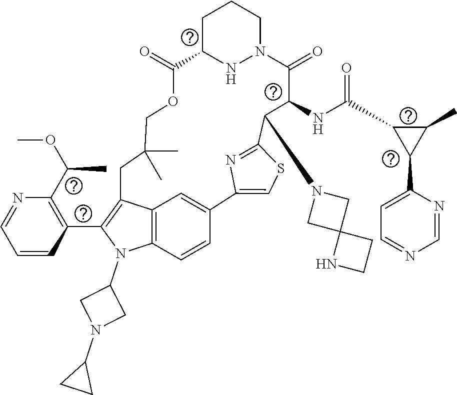 |
| A1017 | 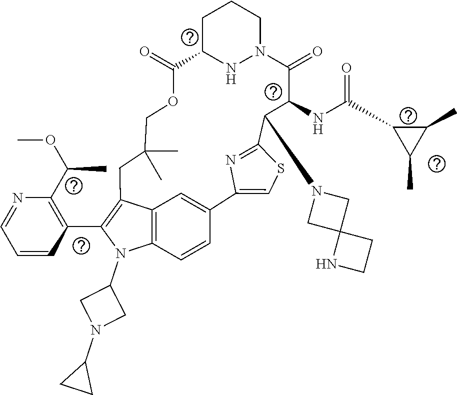 |
| A1018 | 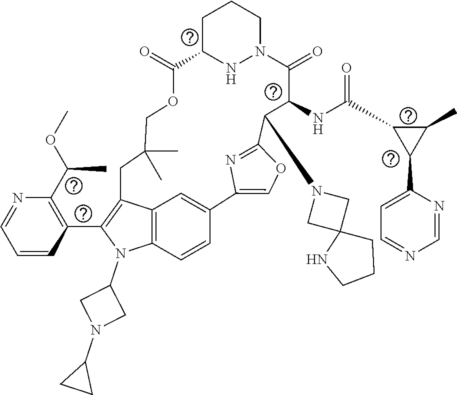 |
| A1019 | 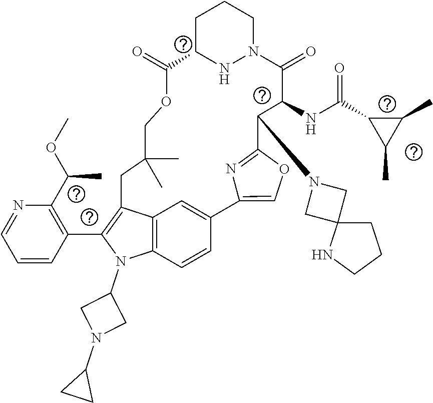 [SMILES: CO[C@@H](C)c1ncccc1-c1c2c3cc(ccc3n1C1CN(C3CC3)C1)-c1coc(n1)[C@@H](N1CC3(CCCN3)C1)[C@H](NC(=O)[C@H]1[C@H](C)[C@@H]1C)C(=O)N1CCC[C@H](N1)C(=O)OCC(C)(C)C2] |
| A1020 | 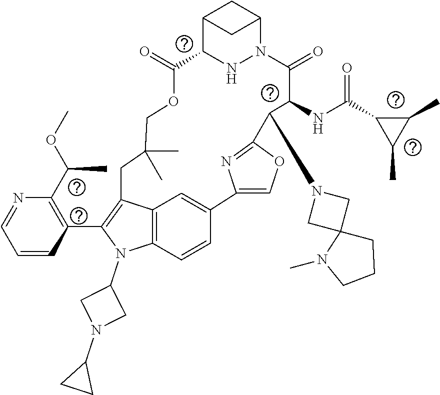 |
| A1021 | 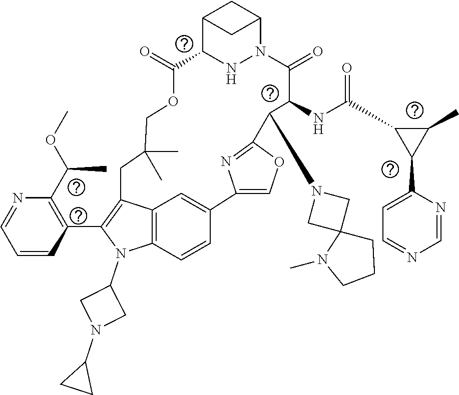 |
| A1022 | 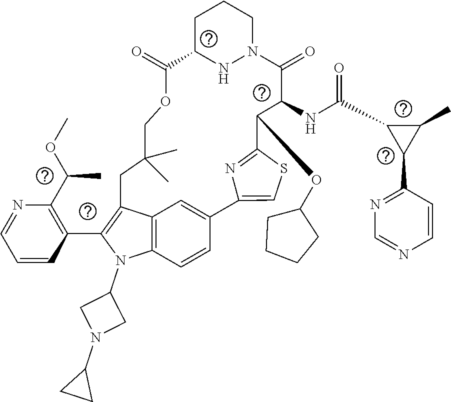 |
| A1023 | 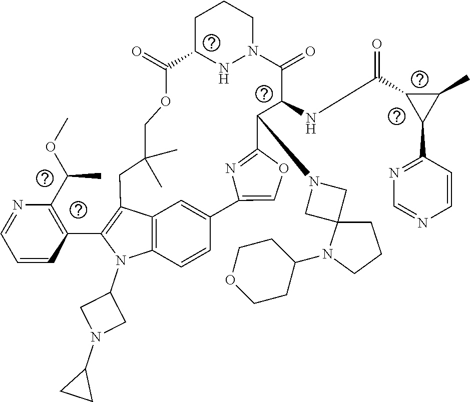 |
| A1024 | 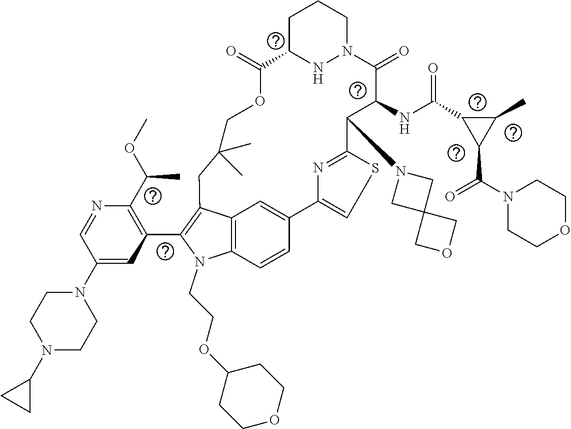 |
| A1025 | 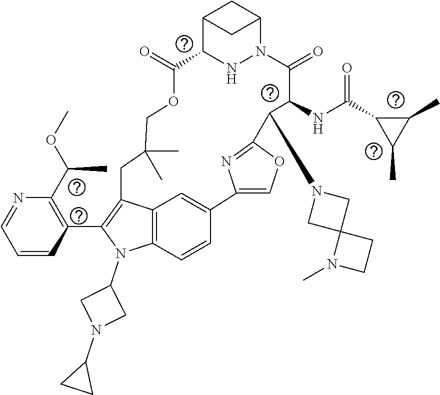 |
| A1026 | 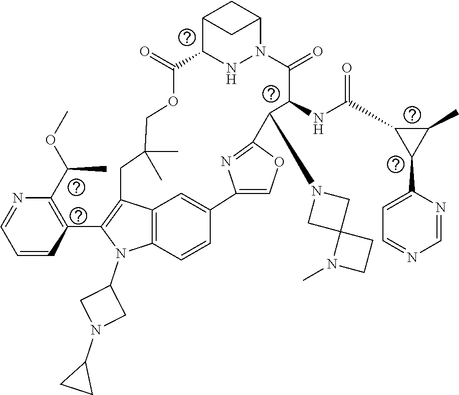 |
| A1027 | 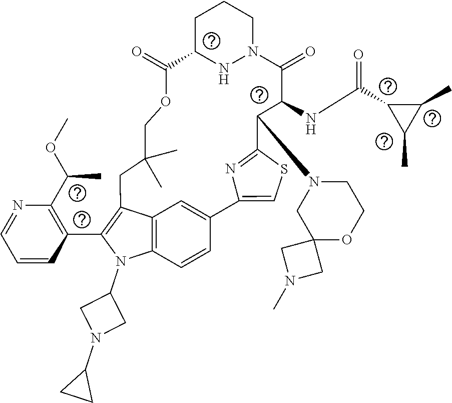 |
| A1028 | 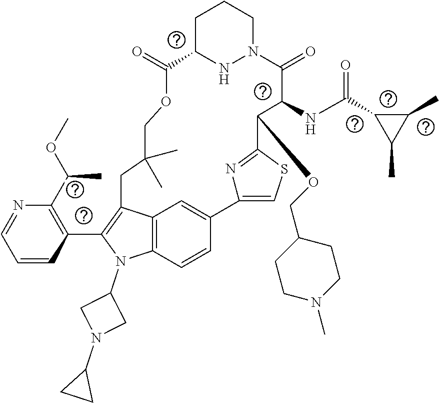 |
| A1029 | 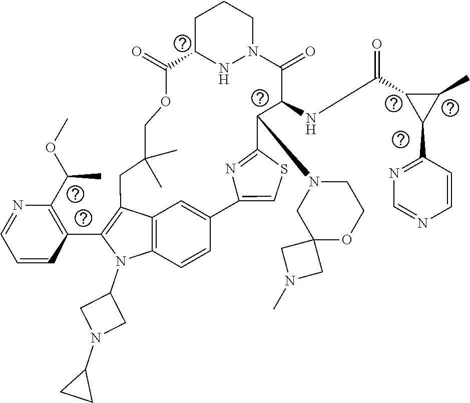 |
| A1030 | 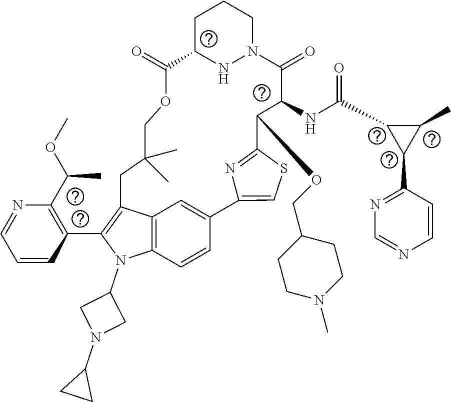 |
| A1031 | 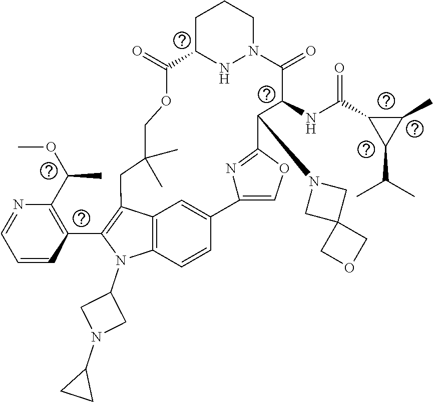 |
| A1032 | 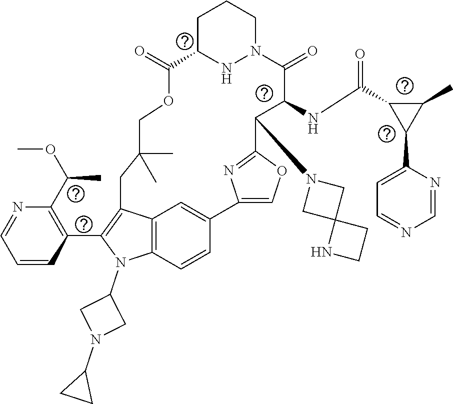 |
| A1033 | 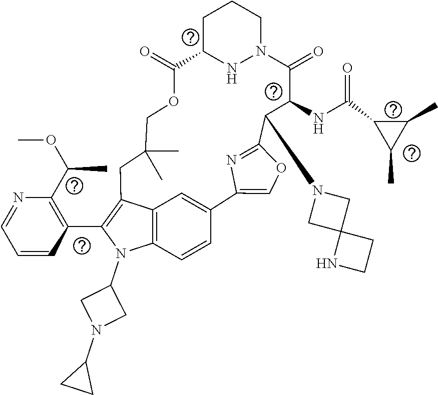 |
| A1034 | 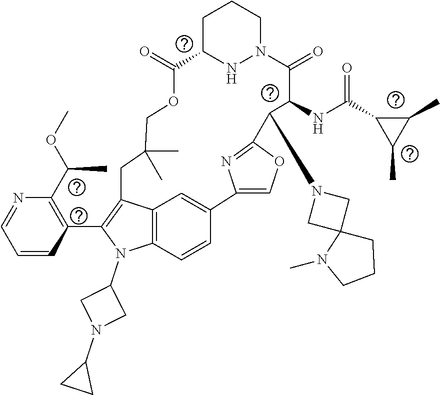 |
| A1035 | 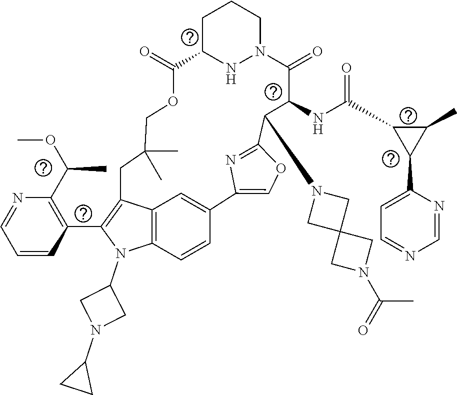 |
| A1036 | 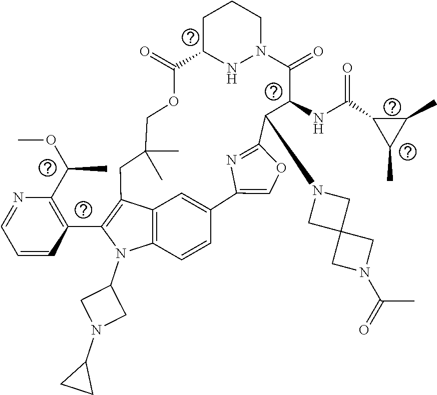 |
| A1037 | 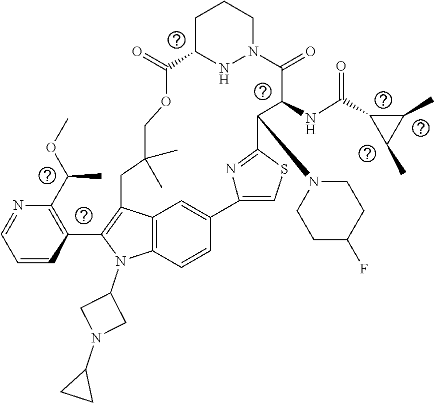 |
| A1038 | 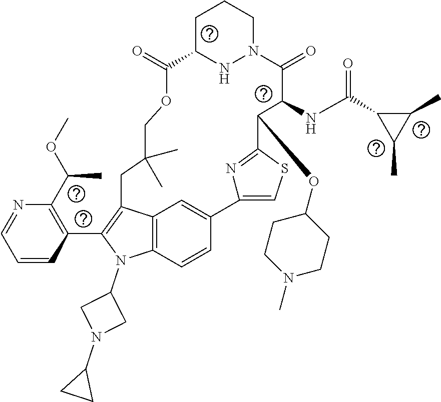 |
| A1039 | 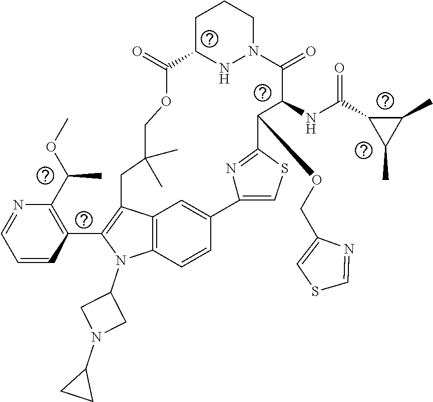 |
| A1040 | 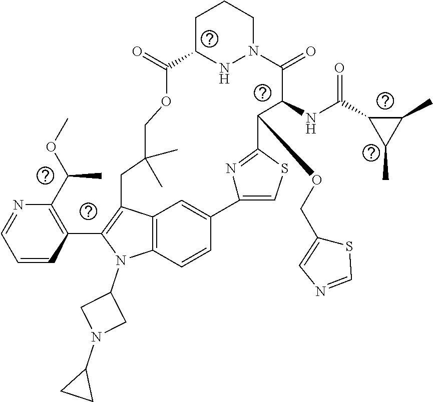 |
| A1041 | 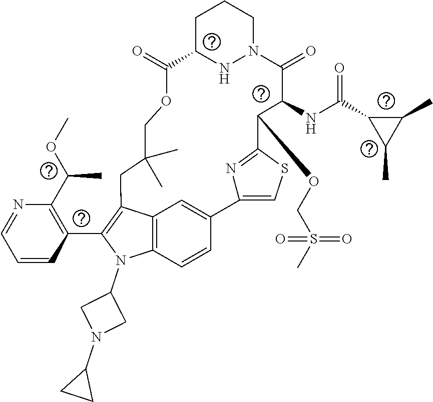 |
| A1042 | 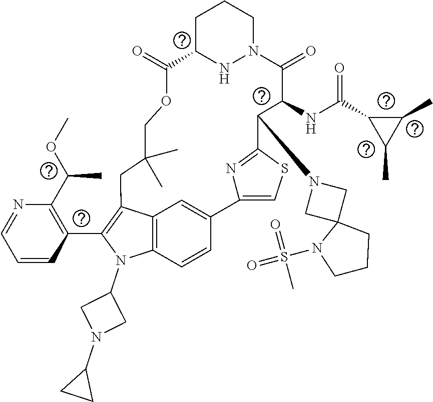 |
| A1043 | 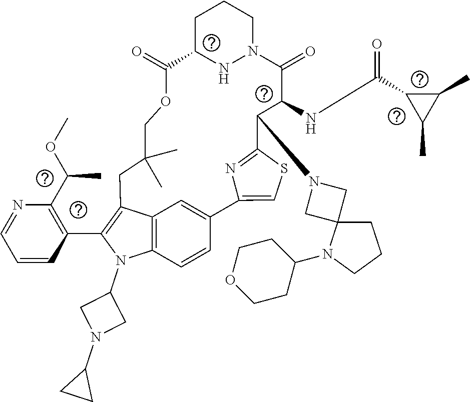 |
| A1044 | 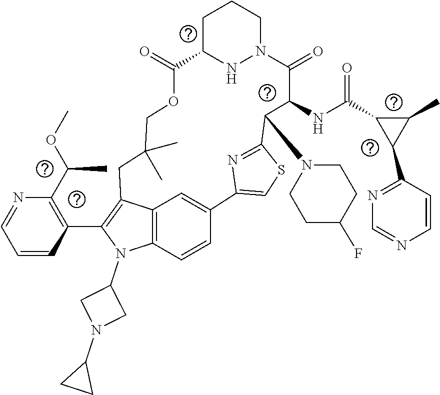 |
| A1045 | 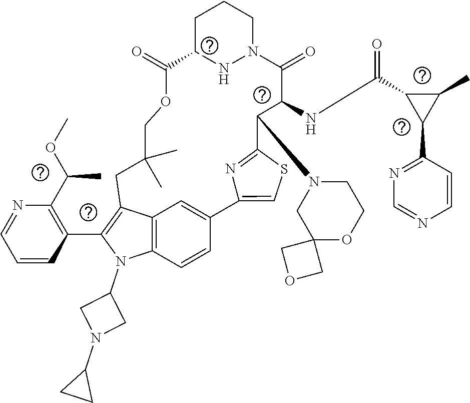 |
| A1046 | 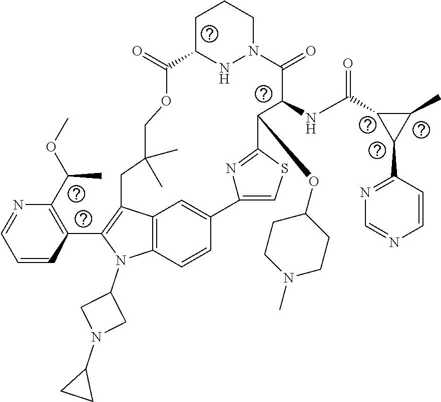 |
| A1047 | 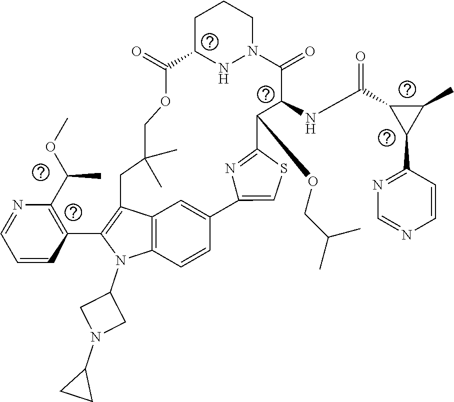 |
| A1048 | 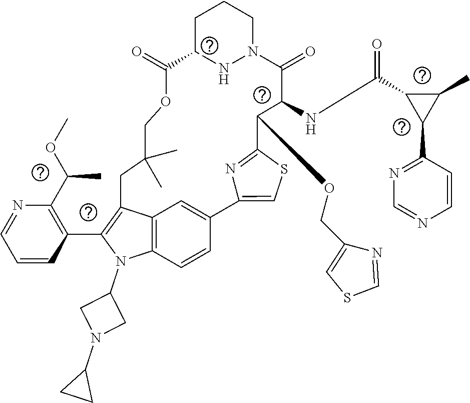 |
| A1049 | 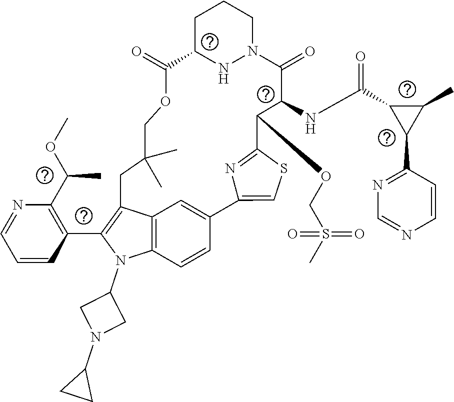 |
| A1050 | 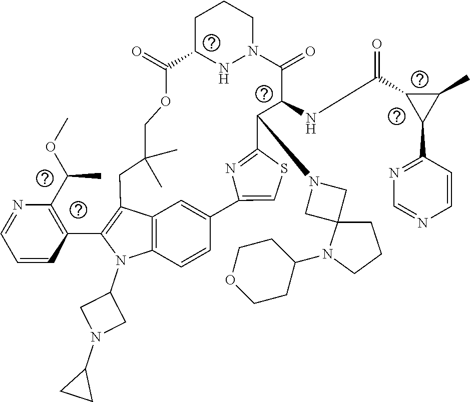 |
| A1051 | 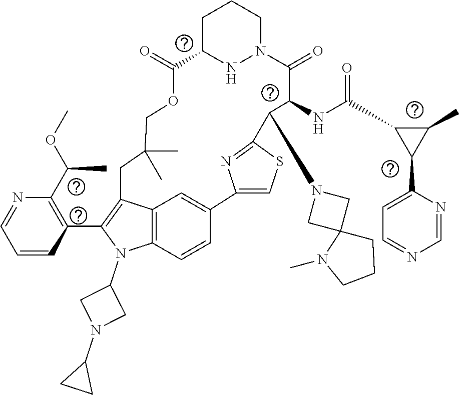 |
| A1052 | 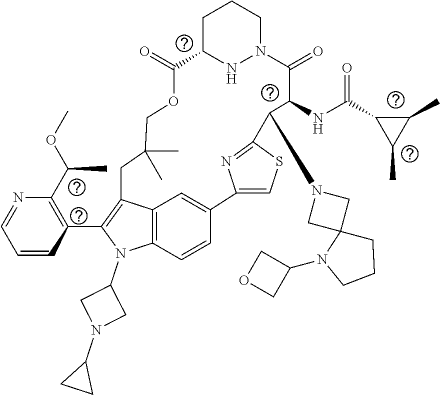 |
| A1053 | 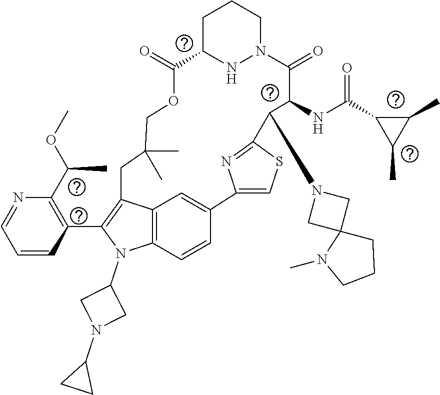 |
| A1054 | 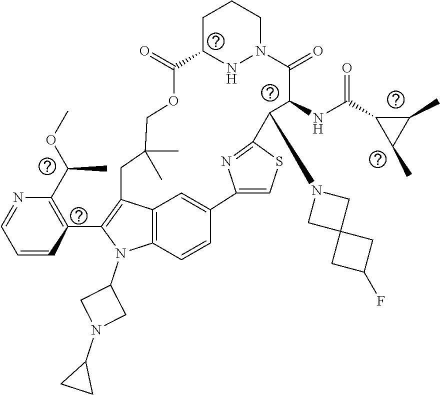 |
| A1055 | 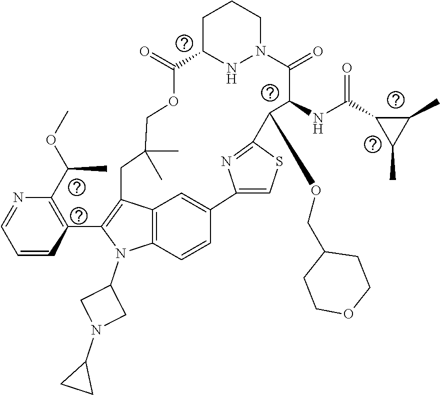 |
| A1056 | 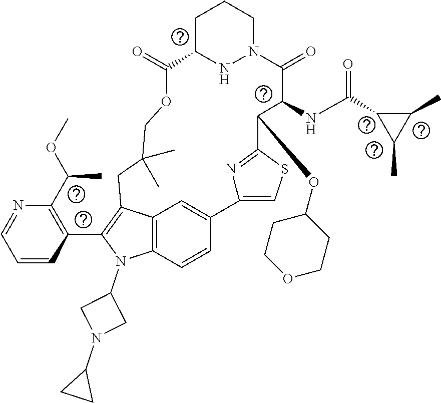 |
| A1057 | 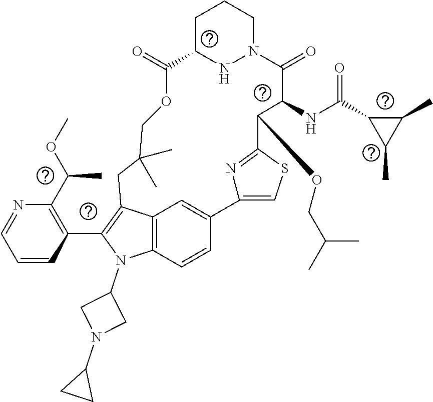 |
| A1058 | 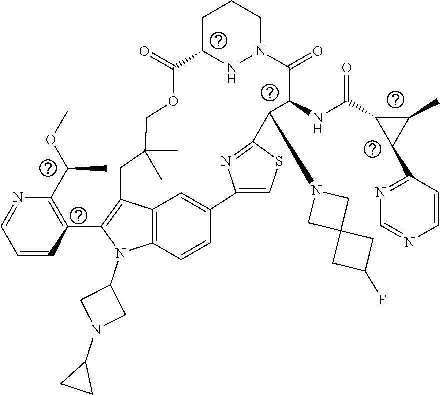 |
| A1059 | 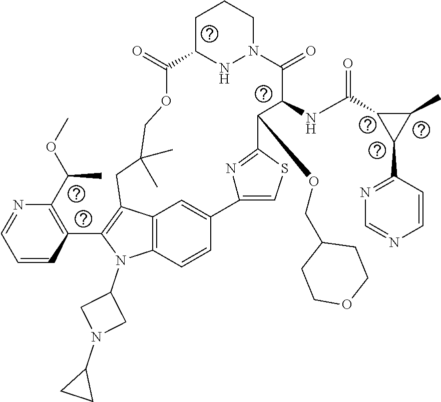 |
| A1060 | 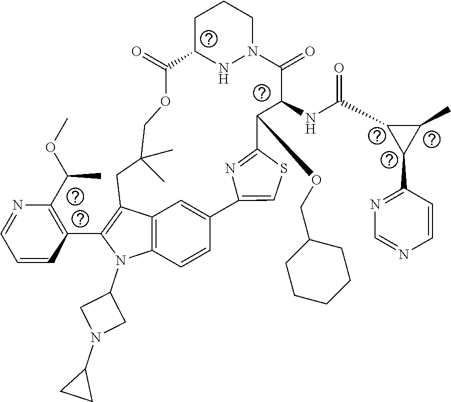 |
| A1061 | 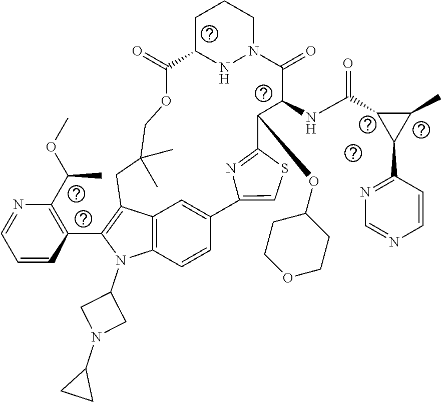 |
| A1062 | 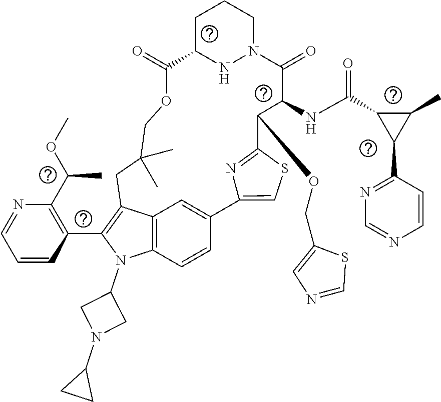 |
| A1063 | 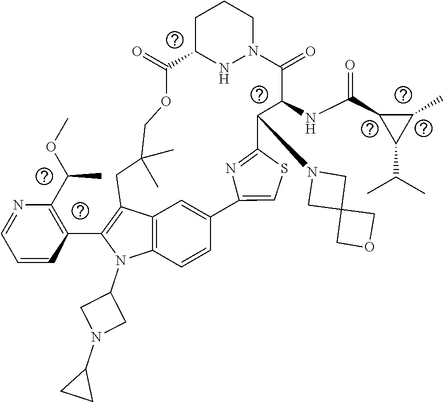 |
| A1064 | 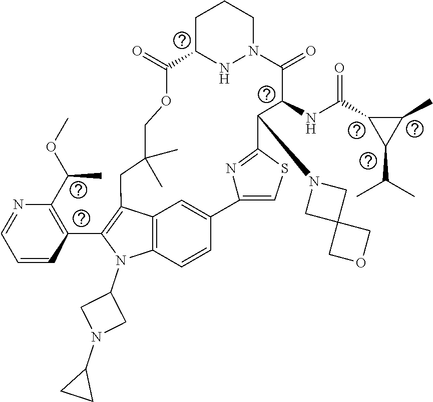 |
| A1065 | 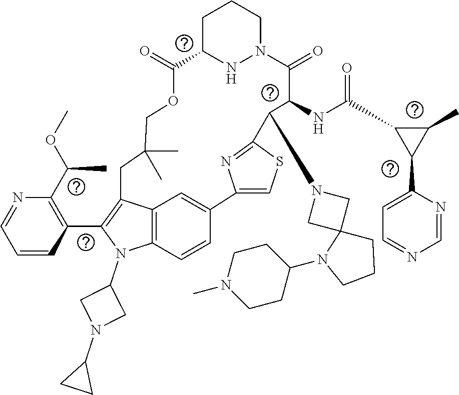 |
| A1066 | 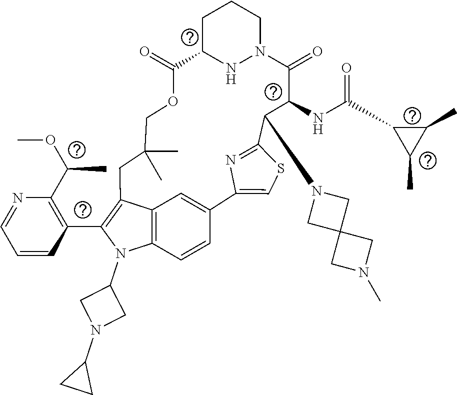 |
| A1067 | 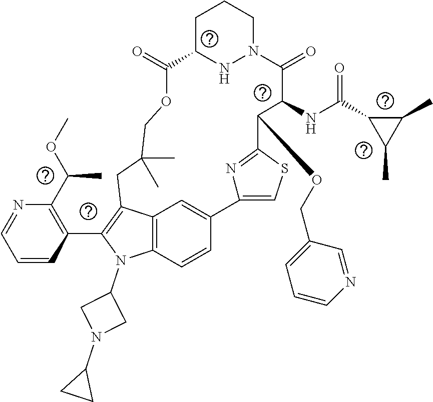 |
| A1068 | 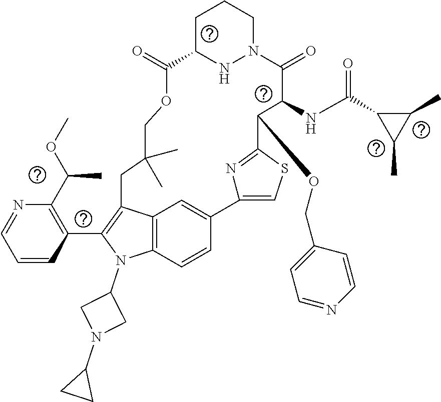 |
| A1069 | 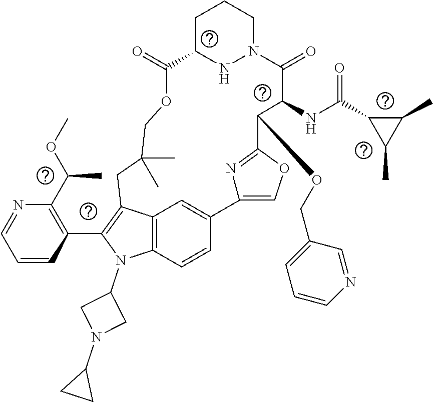 |
| A1070 | 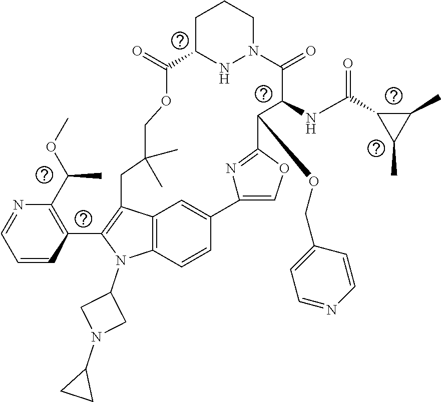 |
| A1071 | 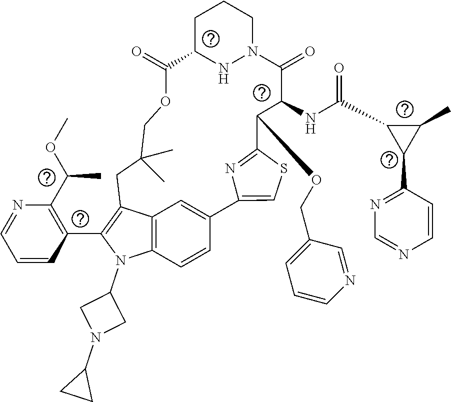 |
| A1072 | 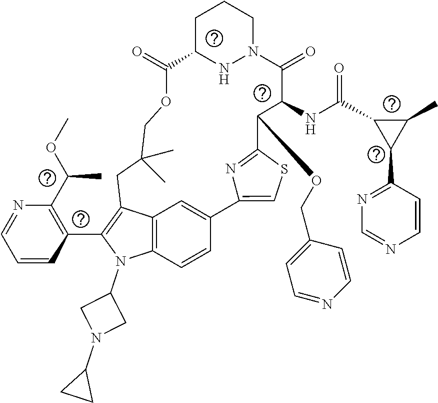 |
| A1073 | 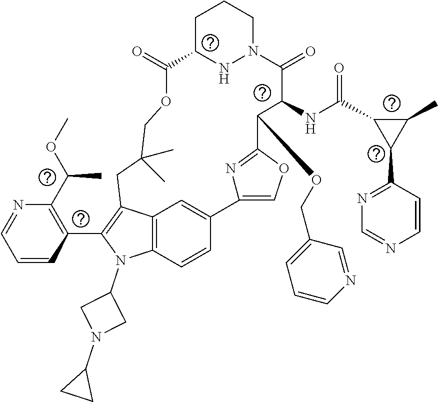 |
| A1074 | 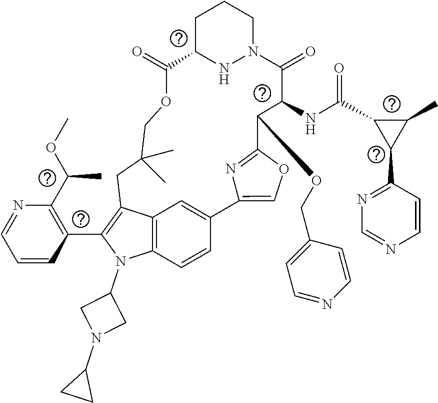 |
| A1075 | 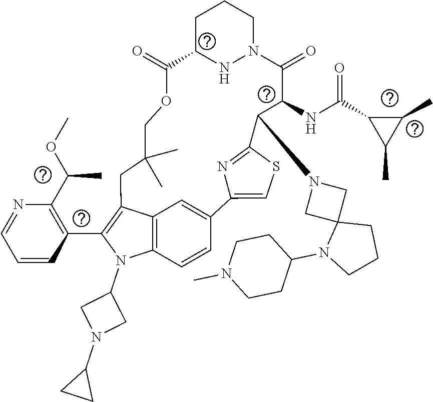 |
| A1076 | 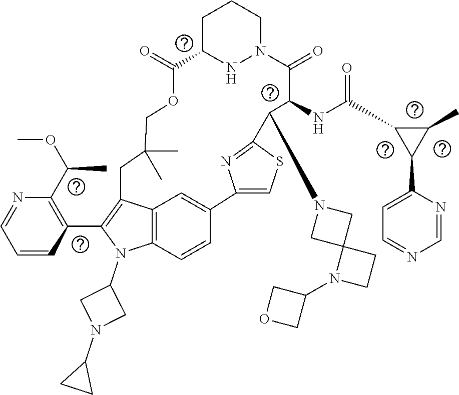 |
| A1077 | 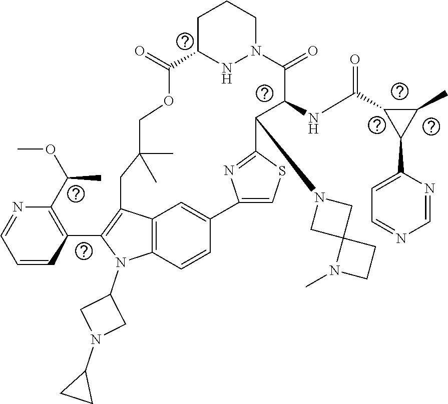 |
| A1078 | 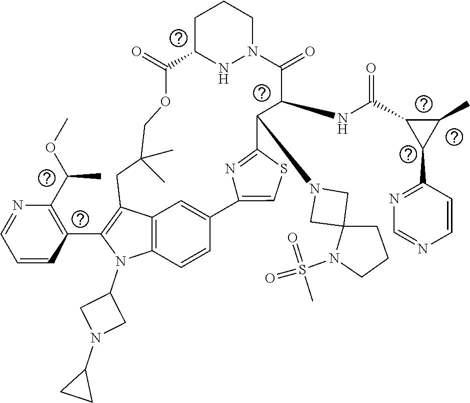 |
| A1079 | 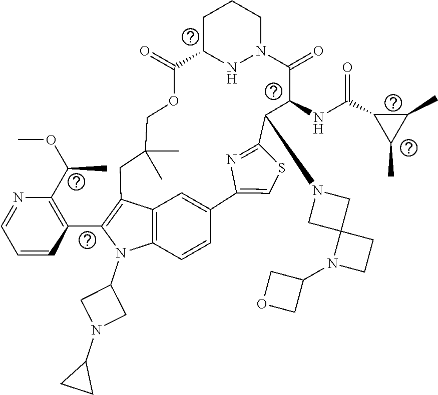 |
| A1080 | 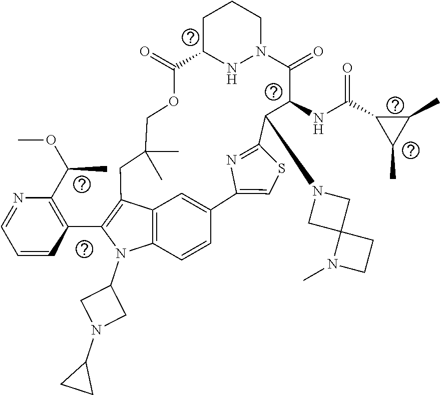 |
| A1081 | 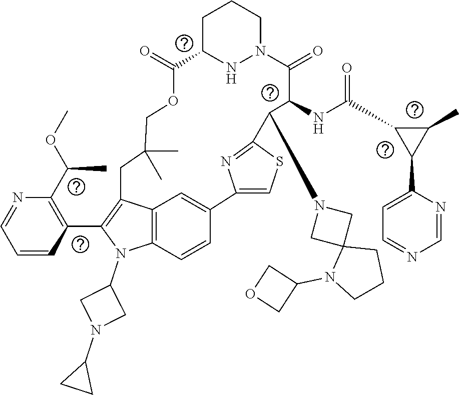 |
| A1082 | 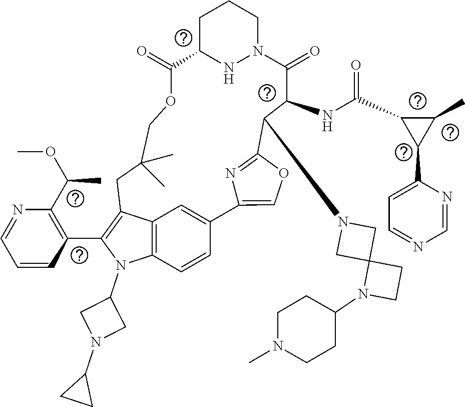 |
| A1083 | 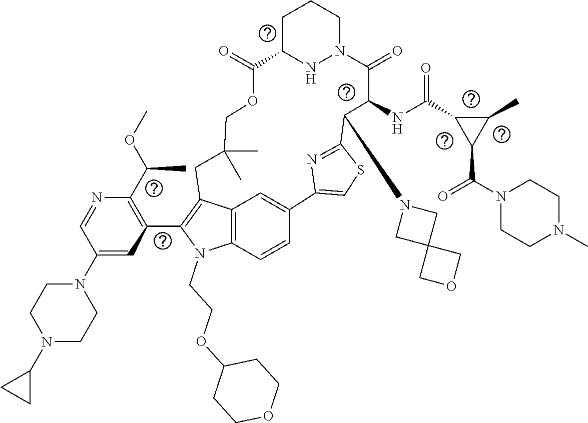 |
| A1084 | 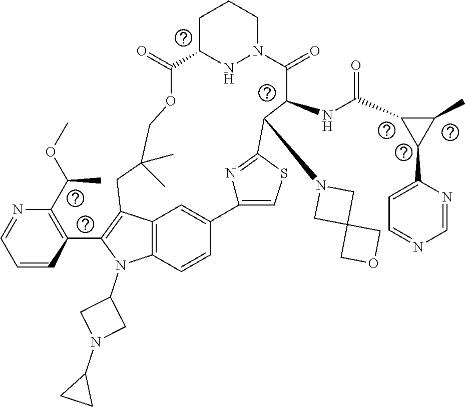 |
| A1085 | 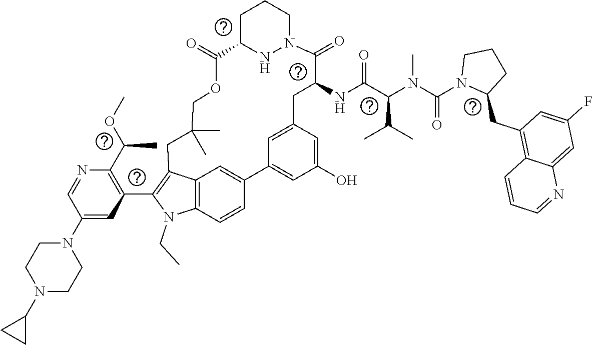 |
| A1086 | 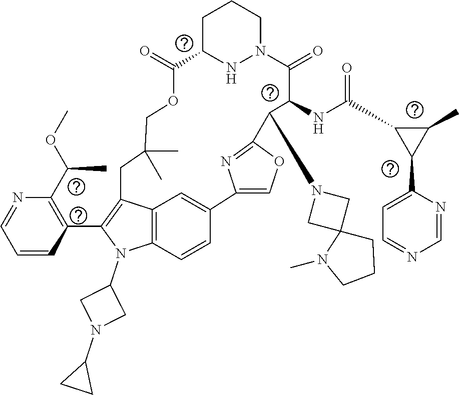 |
| A1087 | 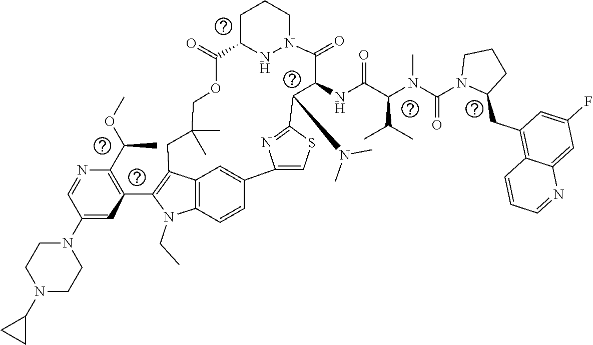 |
| A1088 | 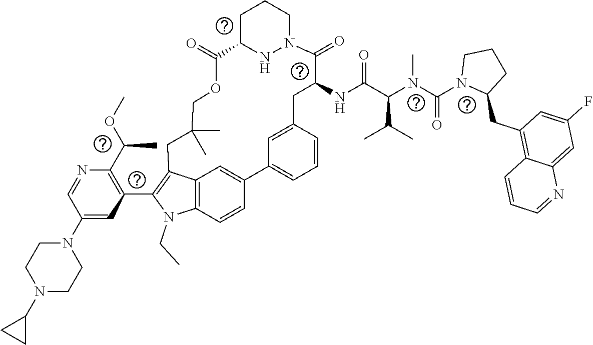 |
| A1089 | 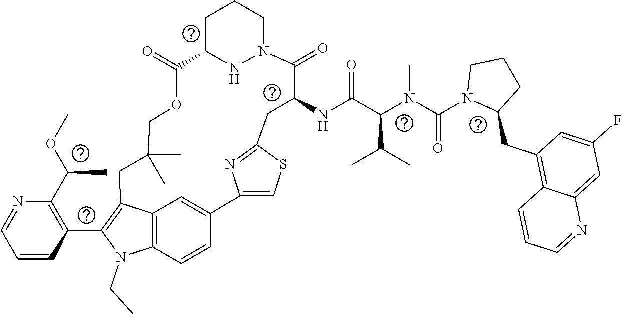 |
| A1090 | 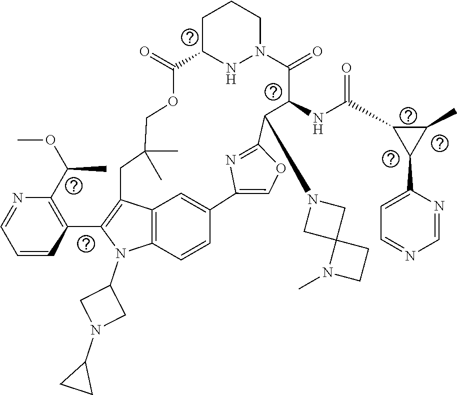 |
| A1091 | 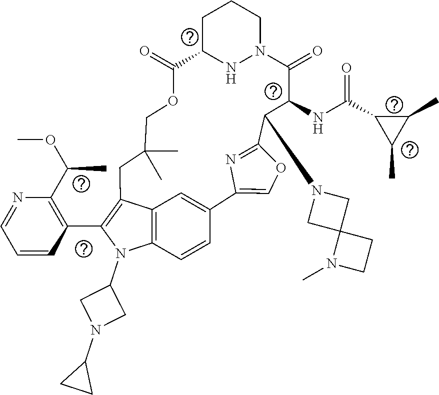 |
| A1092 | 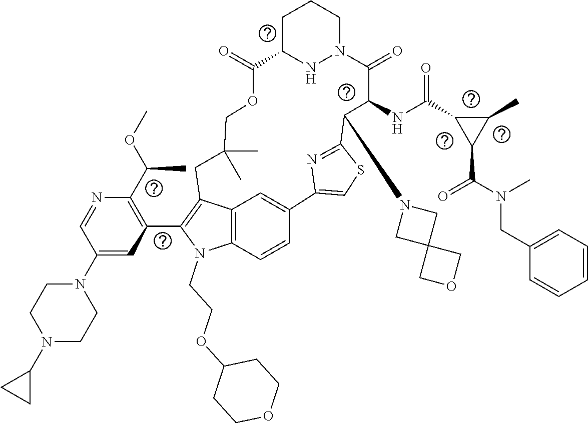 |
| A1093 | 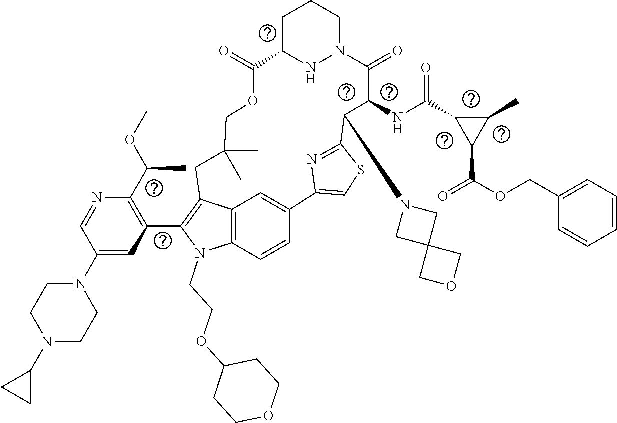 |
| A1094 | 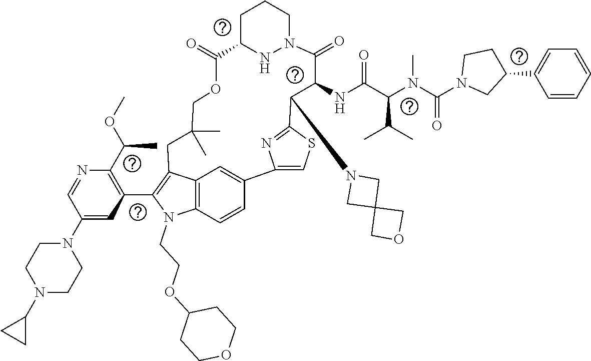 |
| A1095 | 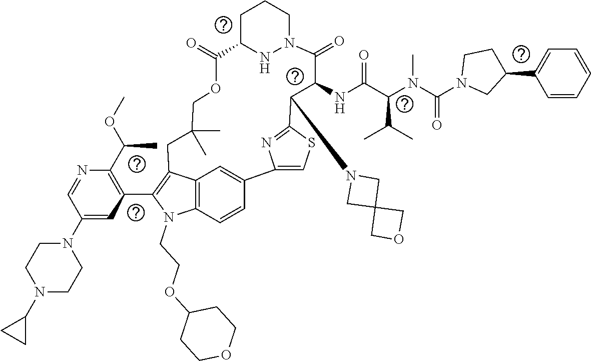 |
| A1096 | 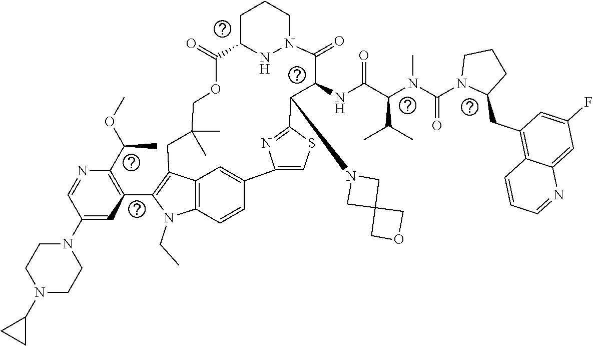 |
| A1097 | 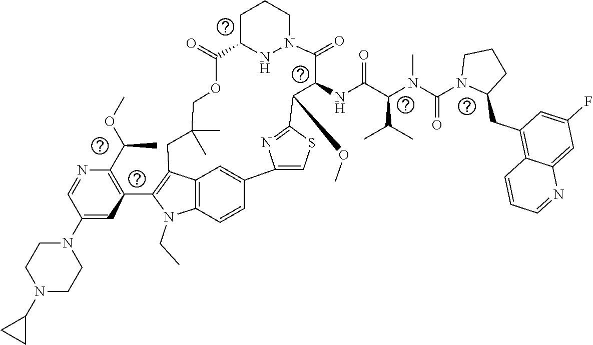 |
| A1098 | 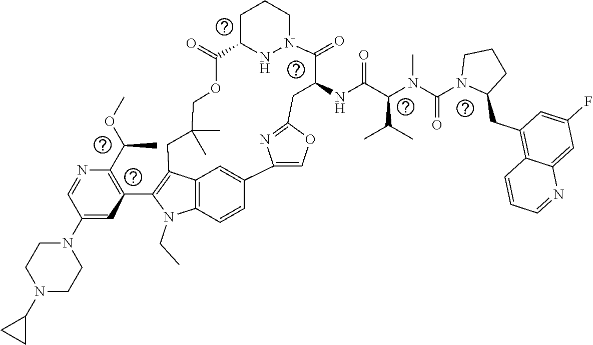 |
| A1099 | 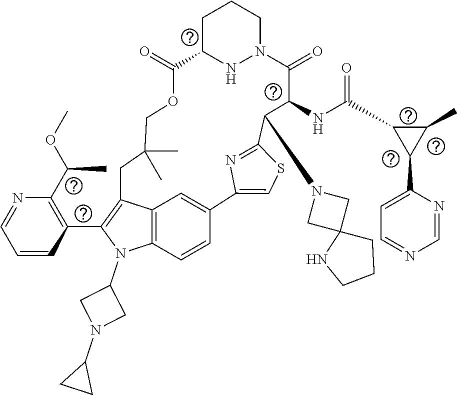 |
| A1100 | 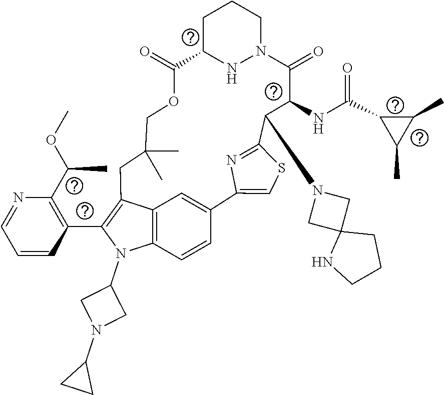 |
| A1101 | 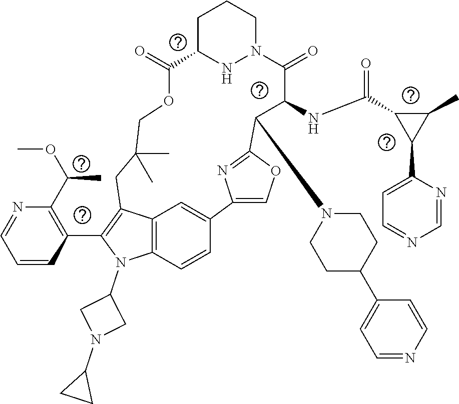 |
| A1102 | 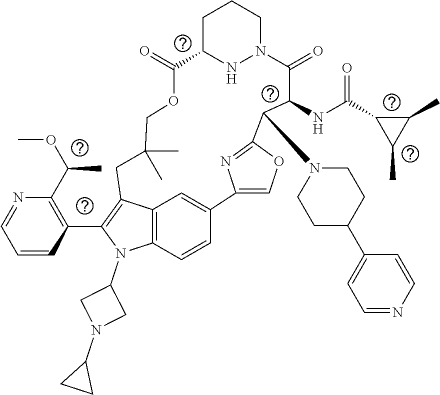 |
| A1103 | 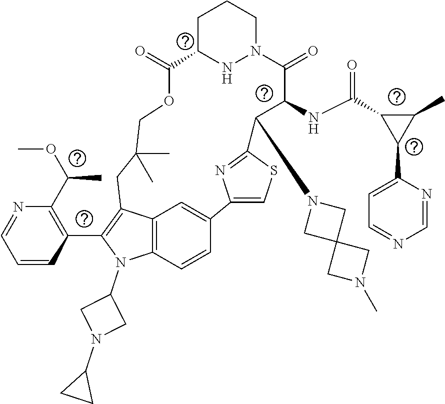 |
| A1104 | 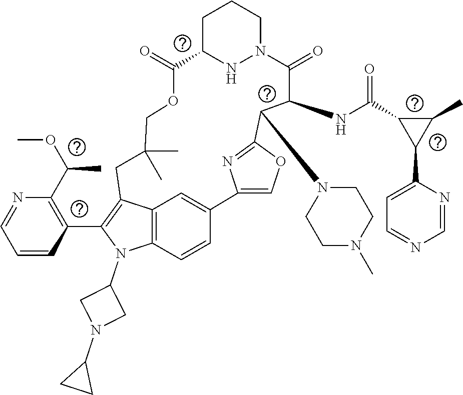 |
| A1105 | 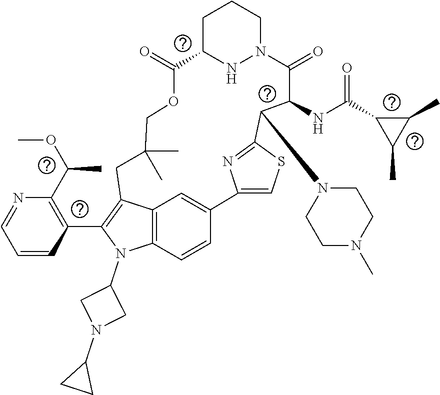 |
| A1106 | 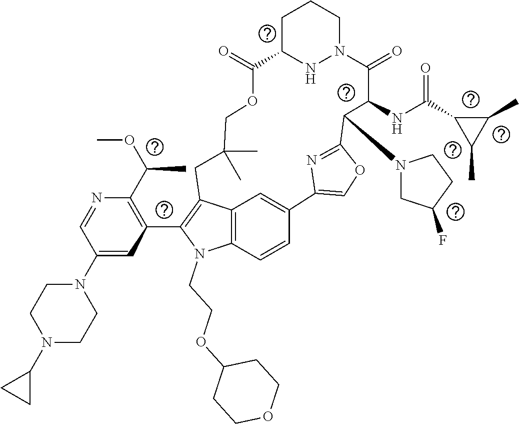 |
| A1107 | 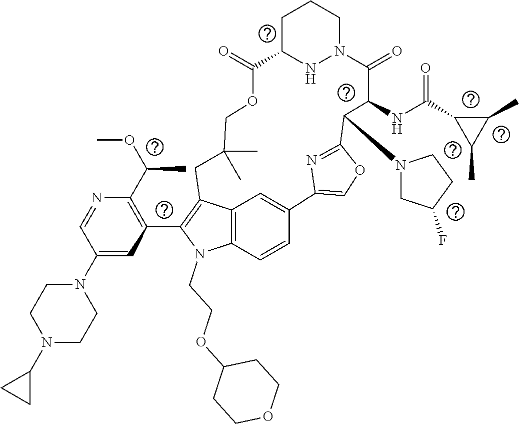 |
| A1108 | 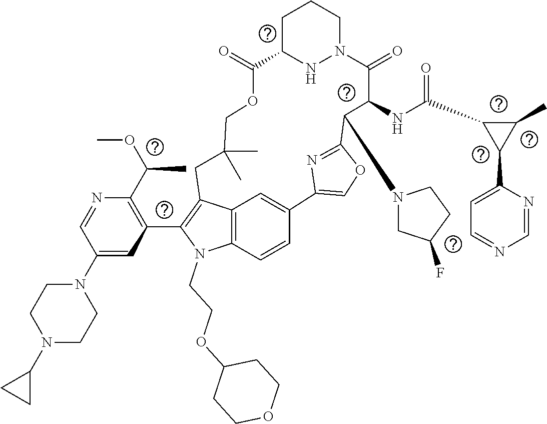 |
| A1109 | 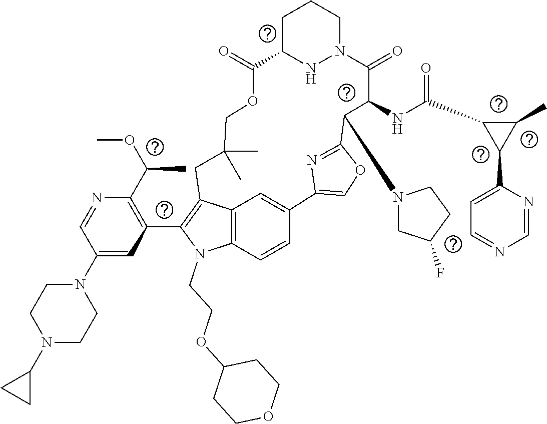 |
| A1110 | 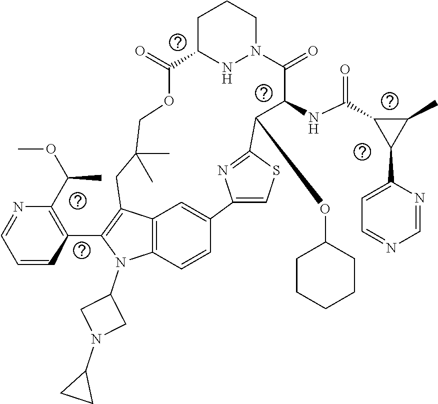 |
| A1111 | 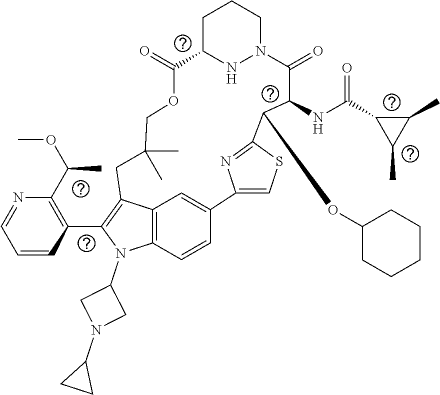 |
| A1112 | 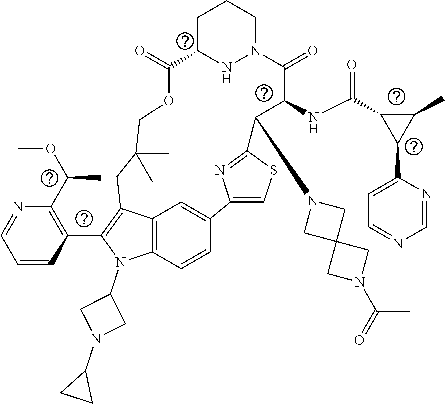 |
| A1113 | 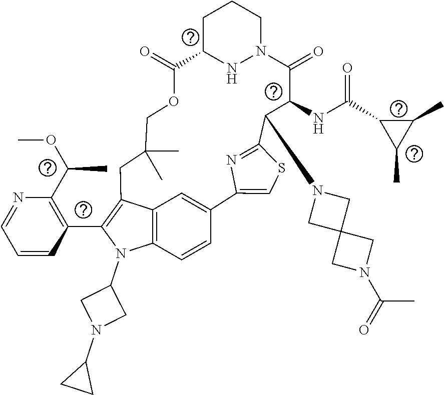 |
| A1114 | 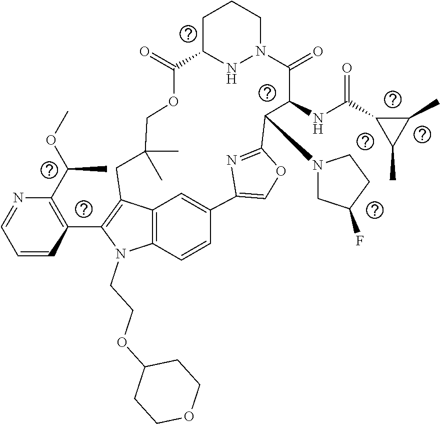 |
| A1115 | 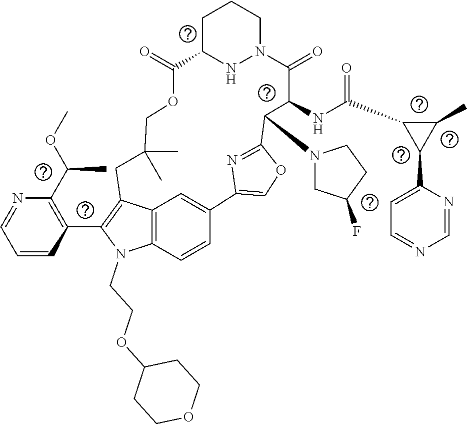 |
| A1116 | 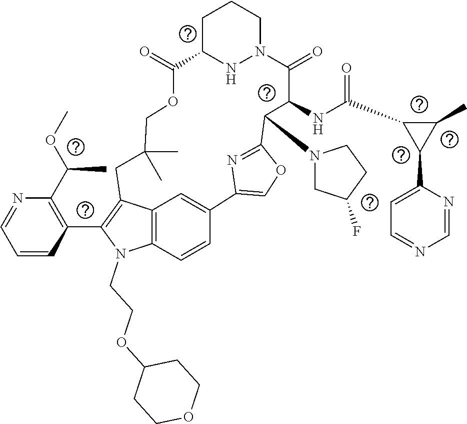 |
| A1117 | 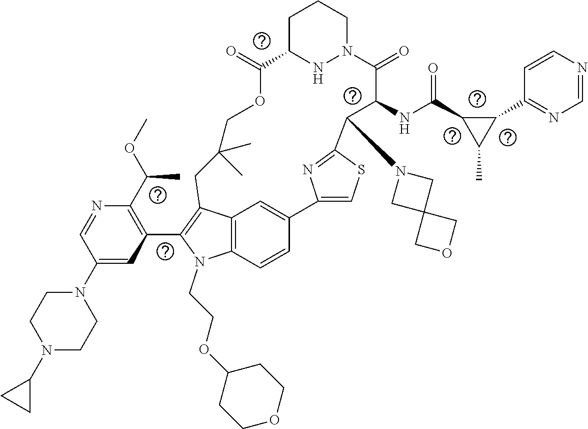 |
| A1118 | 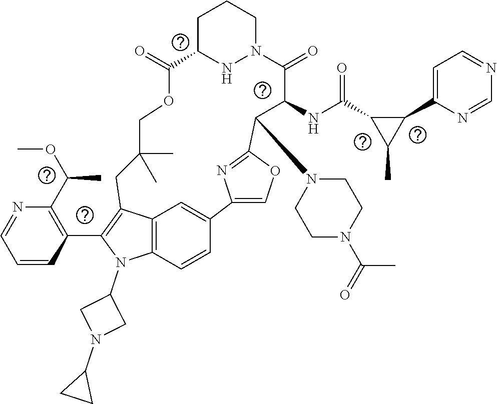 |
| A1119 | 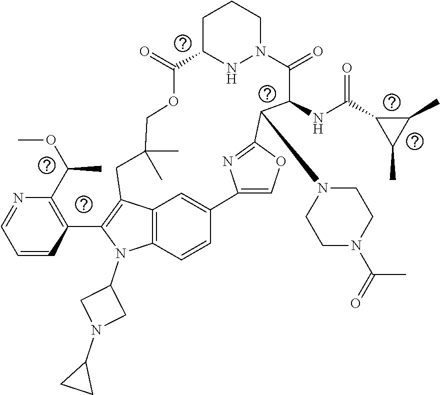 |
| A1120 | 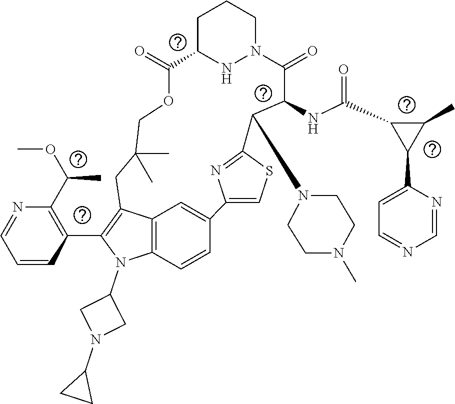 |
| A1121 | 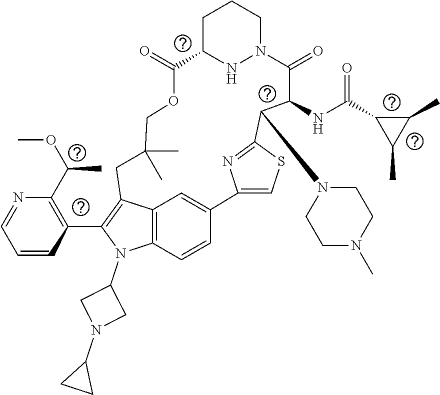 |
| A1122 | 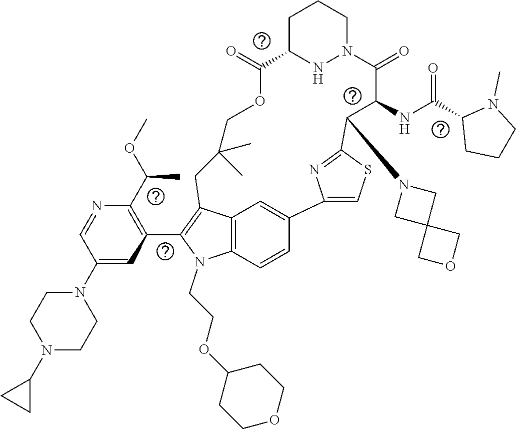 |
| A1123 | 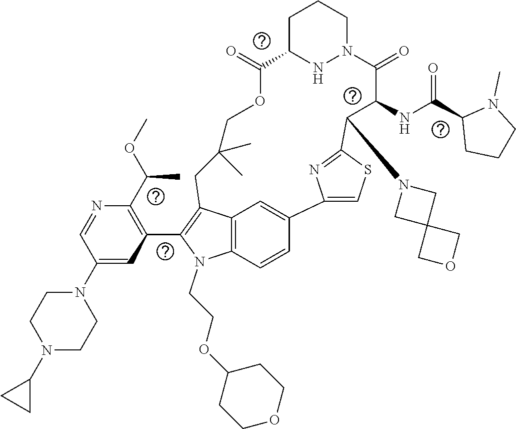 |
| A1124 | 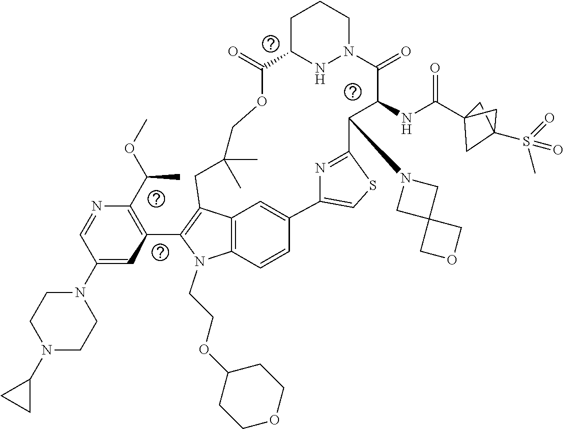 |
| A1125 | 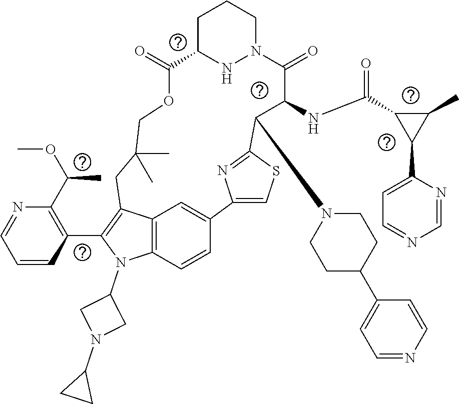 |
| A1126 | 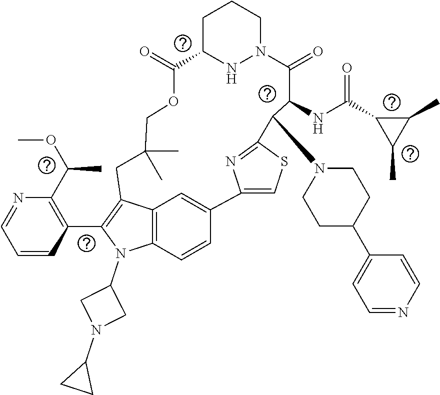 |
| A1127 | 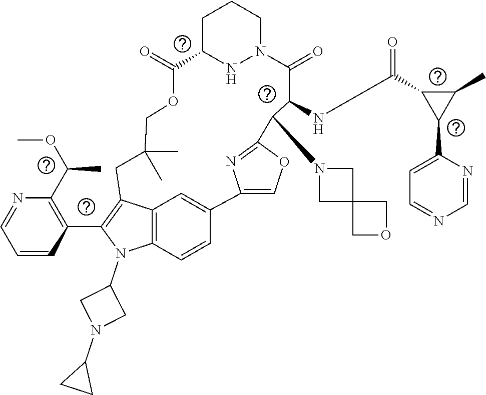 |
| A1128 | 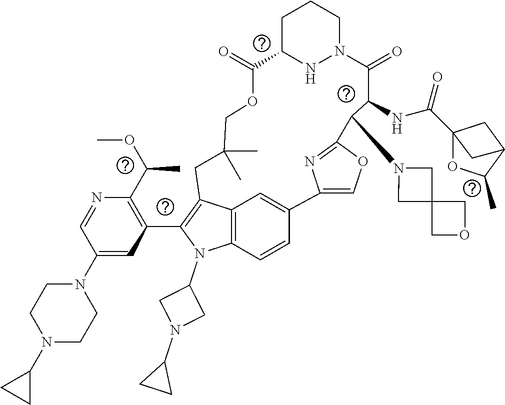 |
| A1129 | 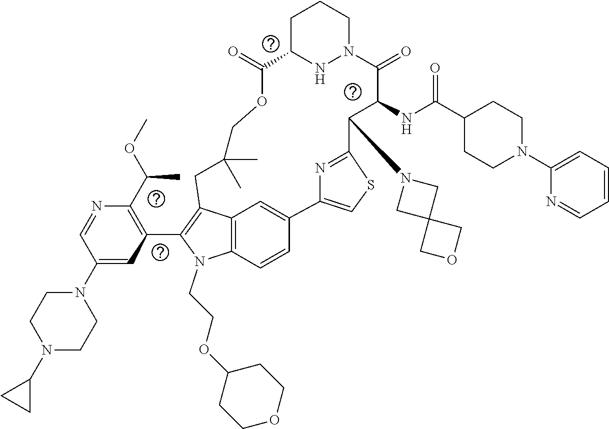 |
| A1130 | 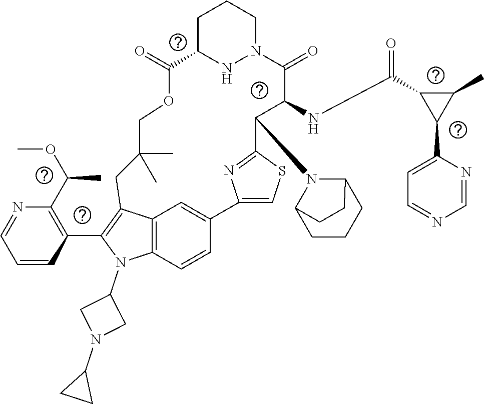 |
| A1131 | 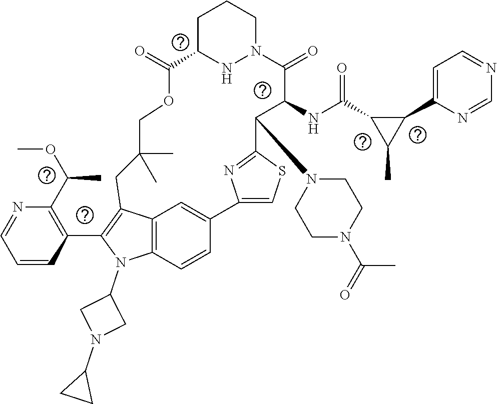 |
| A1132 | 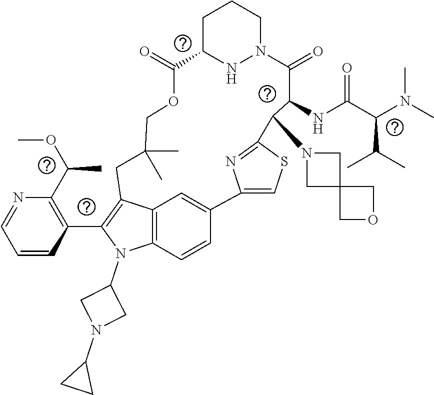 |
| A1133 | 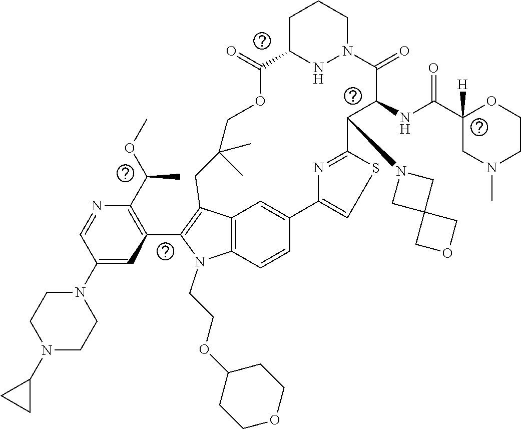 |
| A1134 | 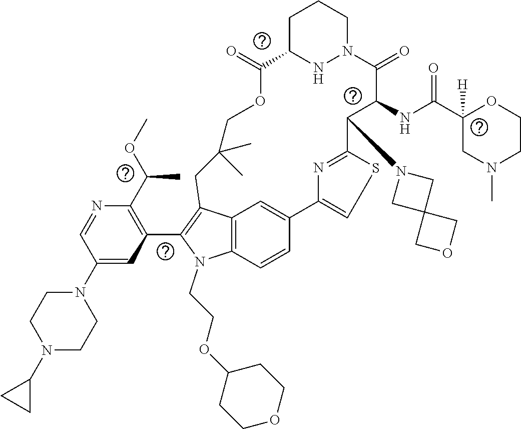 |
| A1135 | 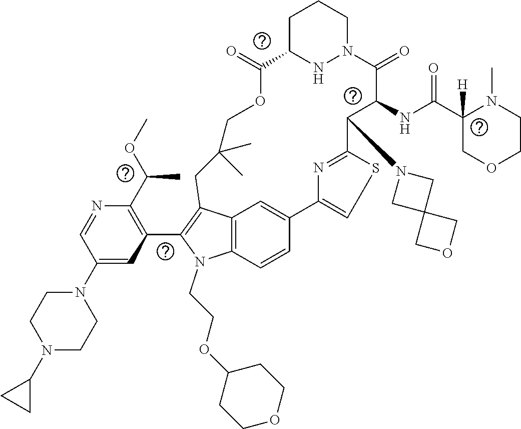 |
| A1136 | 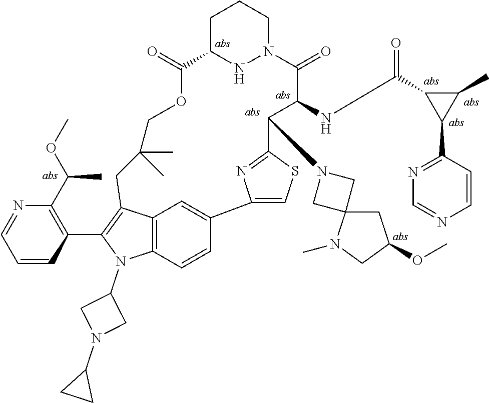 |
| A1137 | 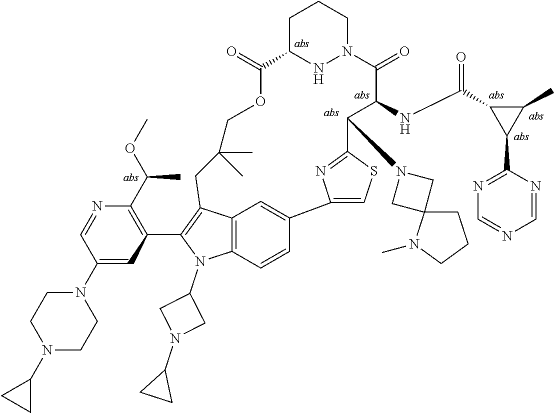 |
| A1138 | 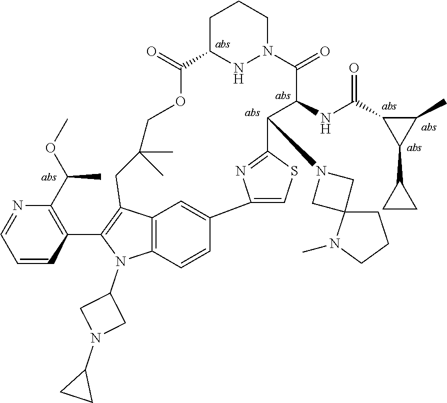 |
| A1139 | 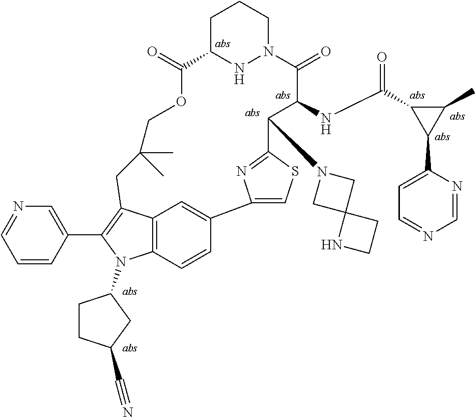 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3csc(n3)[C@H]2N2CC3(CCN3)C2)[C@@H]1c1ccncn1] |
| A1140 | 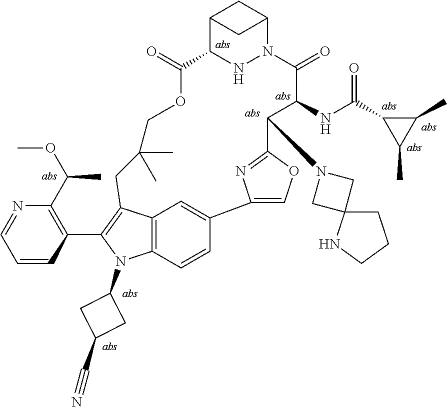 |
| A1141 | 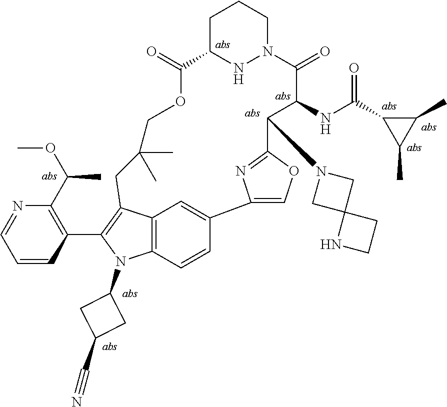 |
| A1142 | 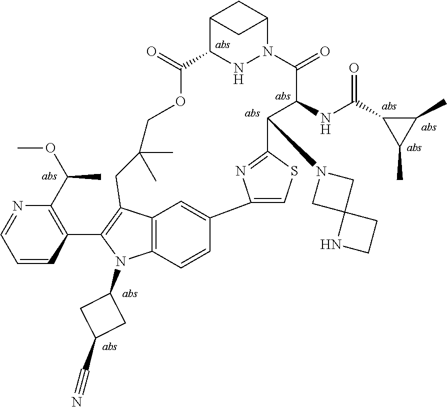 |
| A1143 | 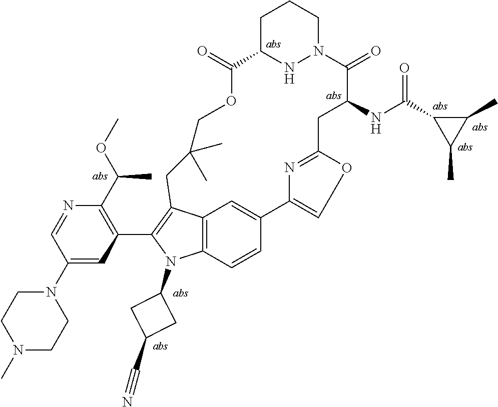 |
| A1144 | 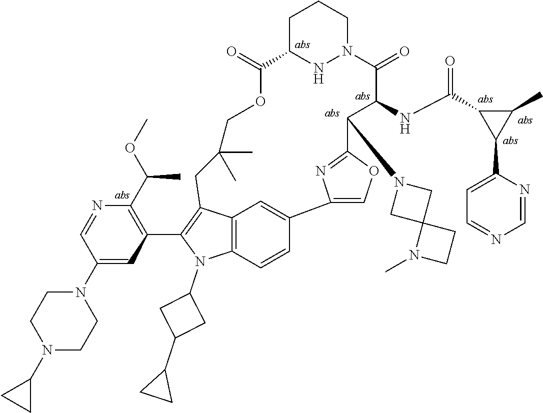 |
| A1145 | 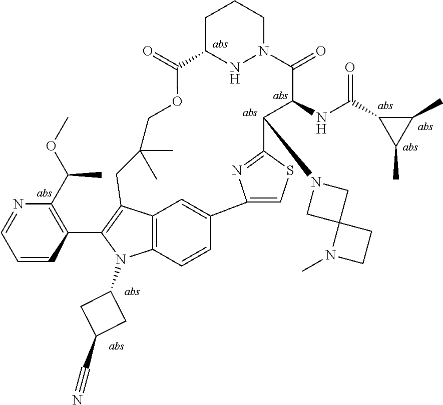 |
| A1146 | 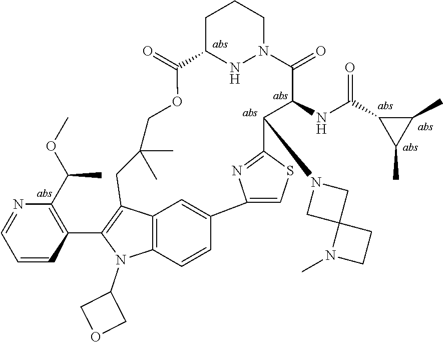 |
| A1147 | 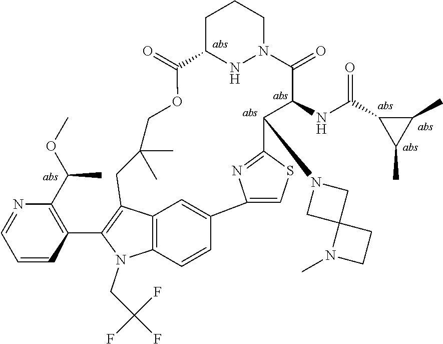 |
| A1148 | 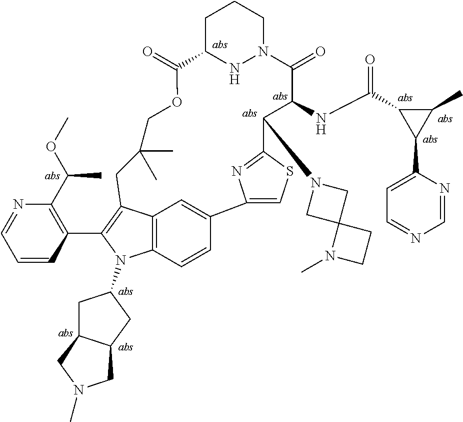 |
| A1149 | 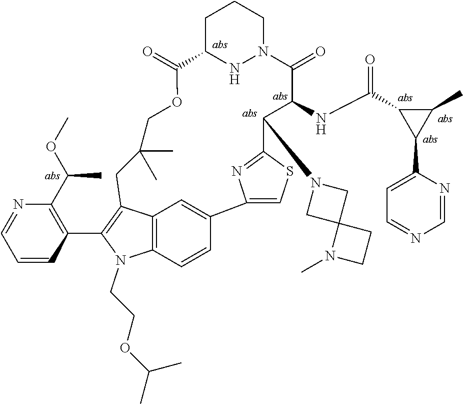 |
| A1150 | 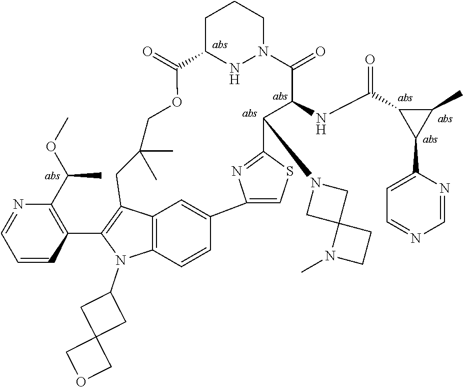 |
| A1151 | 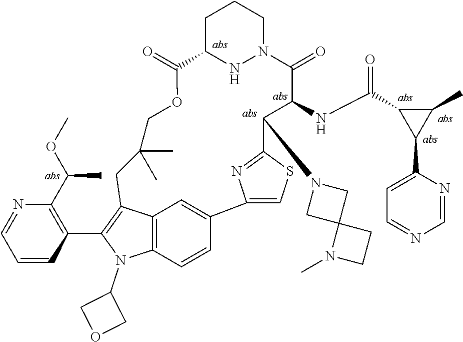 |
| A1152 | 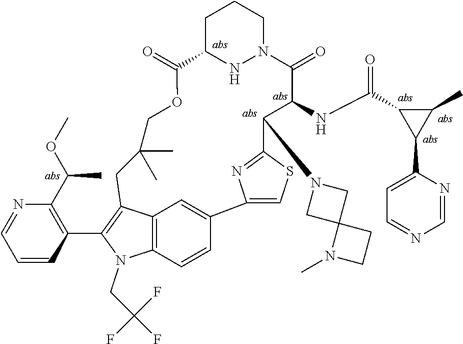 |
| A1153 | 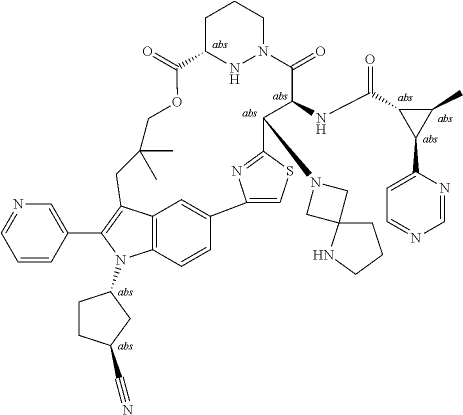 [SMILES: C[C@@H]1[C@@H](C(=O)N[C@@H]2C(=O)N3CCC[C@H](N3)C(=O)OCC(C)(C)Cc3c(-c4cccnc4)n([C@H]4CC[C@H](C#N)C4)c4ccc(cc34)-c3csc(n3)[C@H]2N2CC3(CCCN3)C2)[C@@H]1c1ccncn1] |
| A1154 | 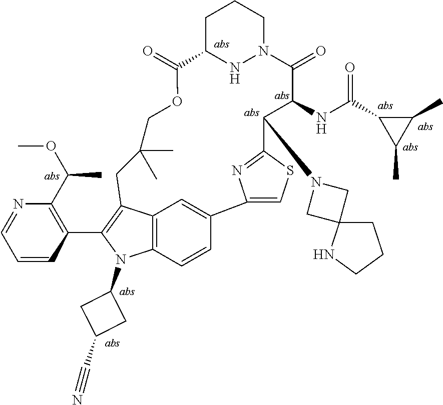 |
| A1155 | 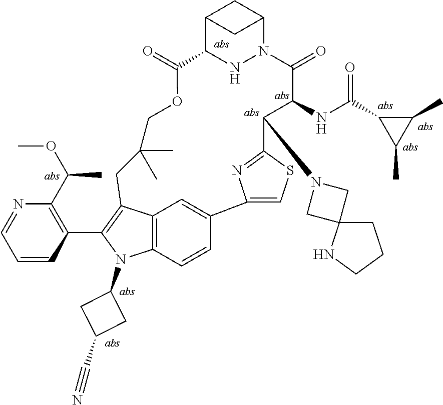 |
| A1156 | 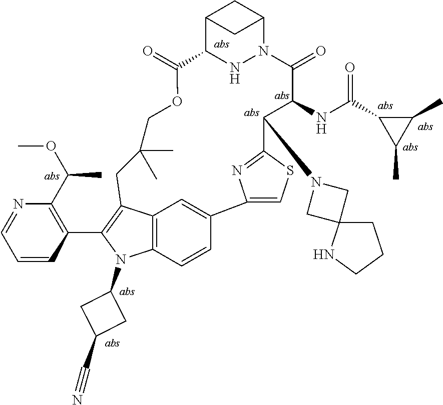 |
| A1157 | 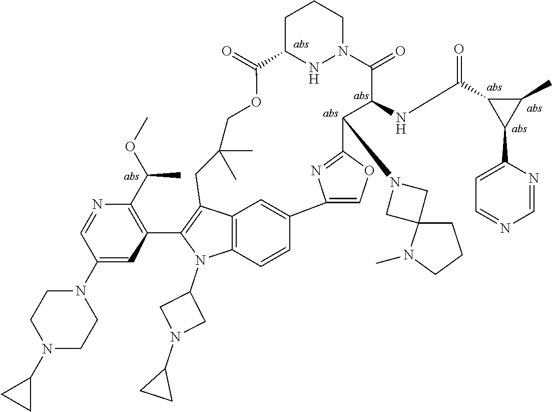 |
| A1158 | 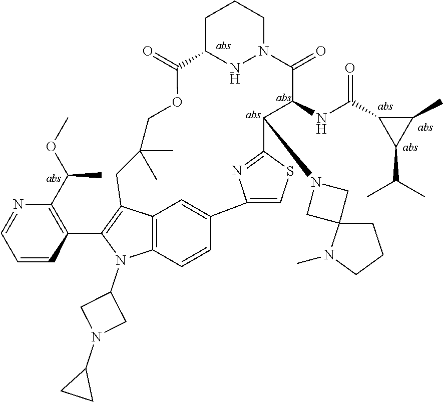 |
| A1159 | 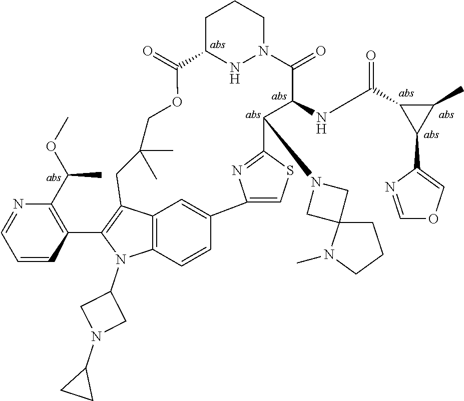 |
| A1160 | 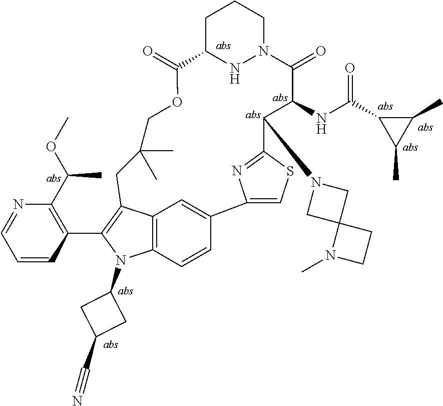 |
| A1161 | 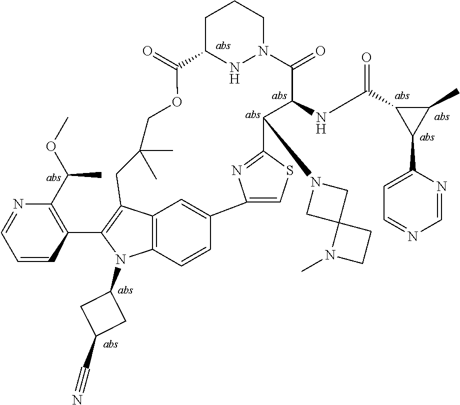 |
| A1162 | 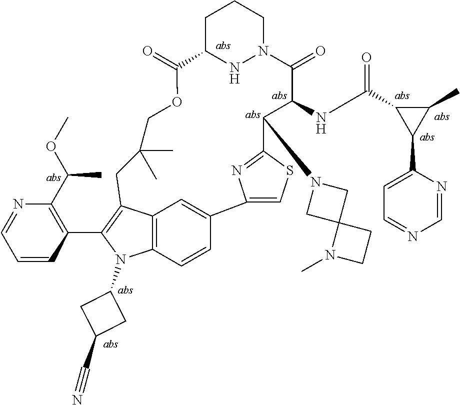 |
| A1163 | 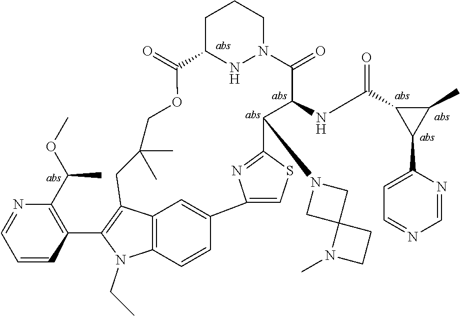 |
| A1164 | 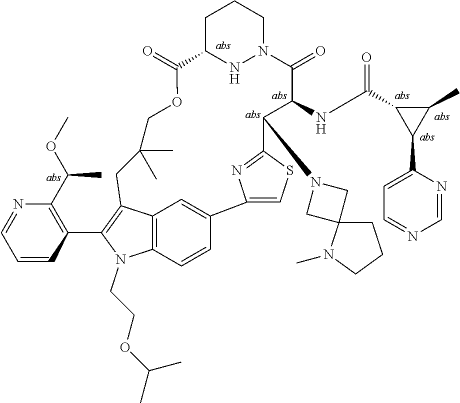 |
| A1165 | 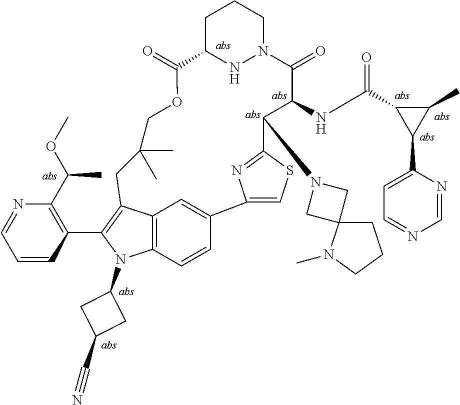 |
| A1166 | 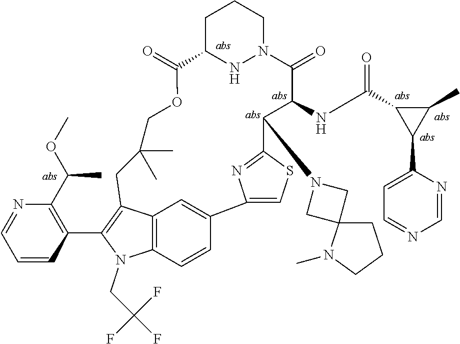 |
| A1167 | 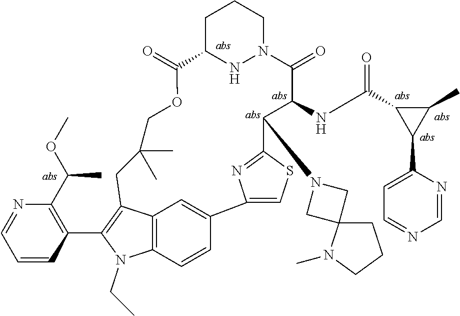 |
| A1168 | 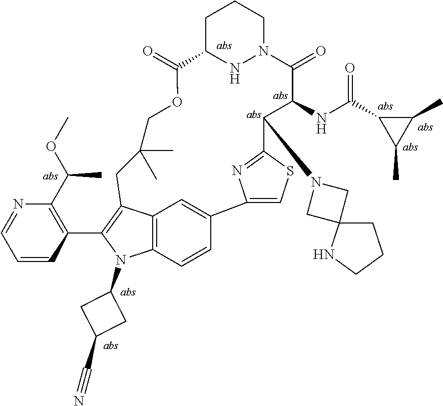 |
| A1169 | 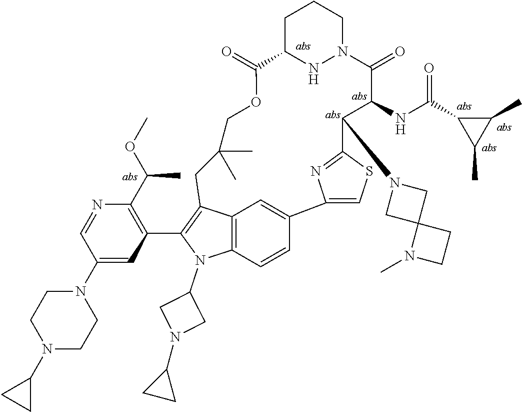 |
| A1170 | 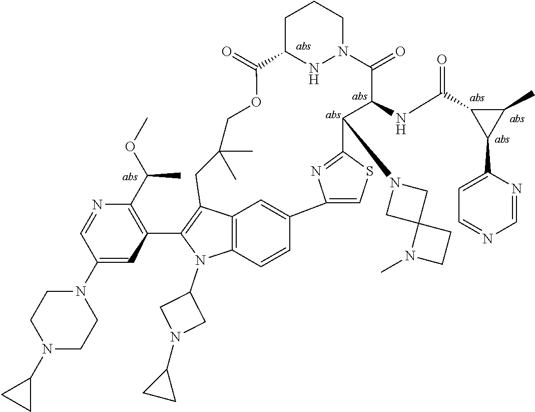 |
| A1171 | 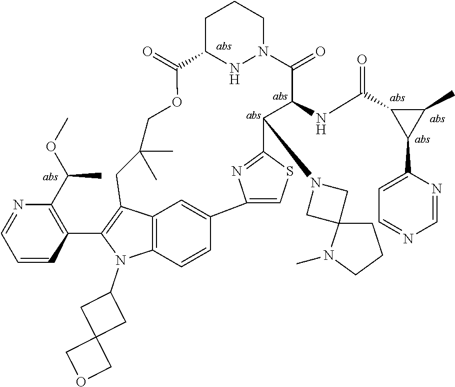 |
| A1172 | 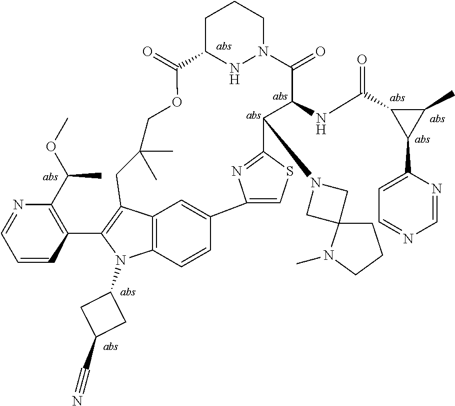 |
| A1173 | 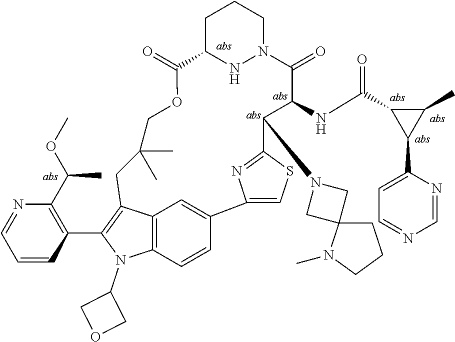 |
| A1174 | 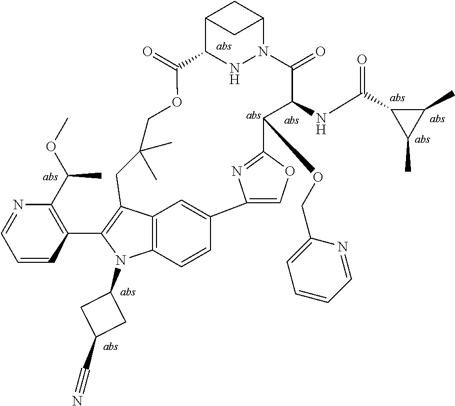 |
| A1175 | 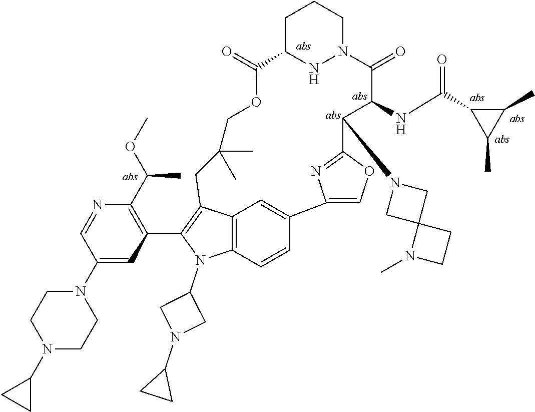 |
| A1176 | 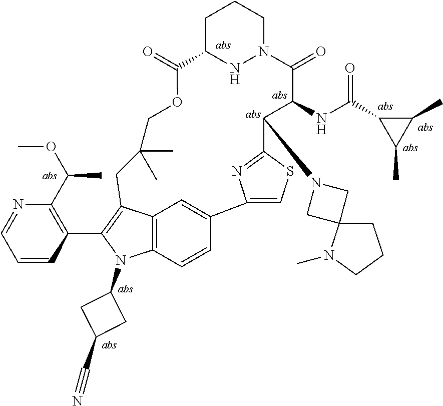 |
| A1177 | 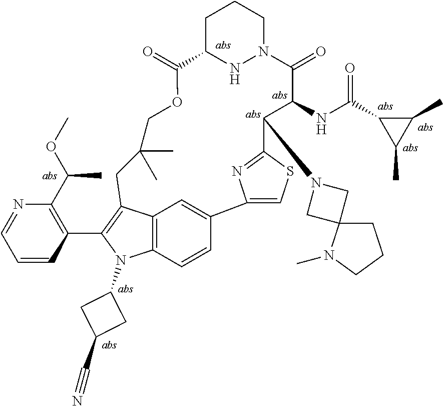 |
| A1178 | 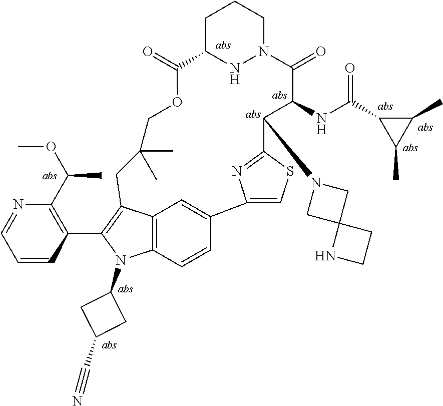 |
| A1179 | 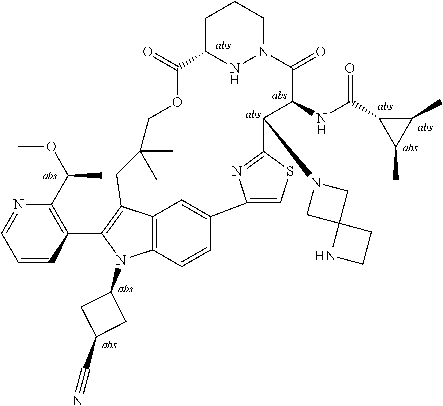 |
| A1180 | 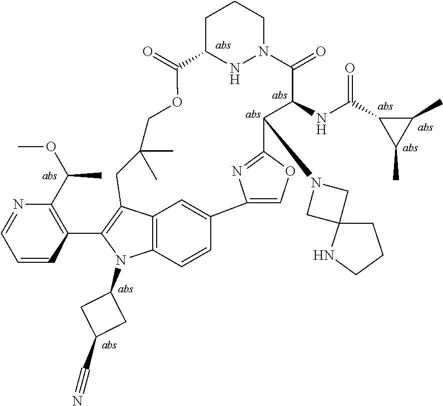 |
| A1181 | 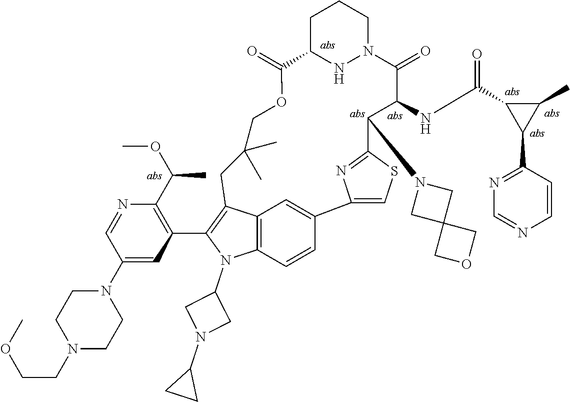 |
| A1182 | 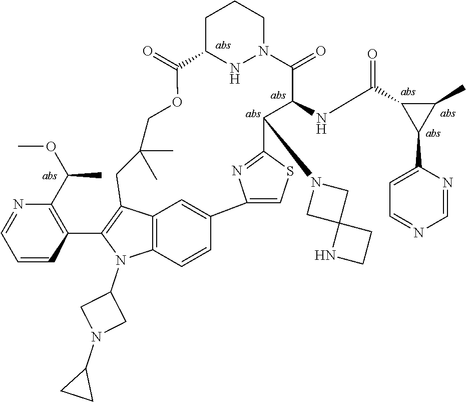 |
| A1183 | 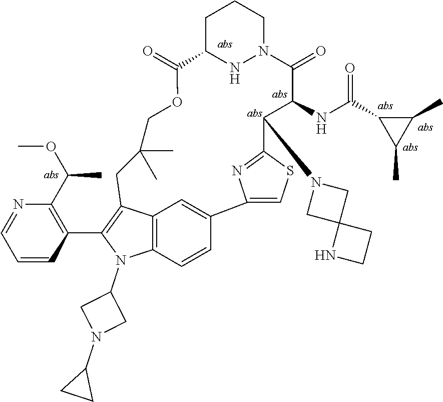 |
| A1184 | 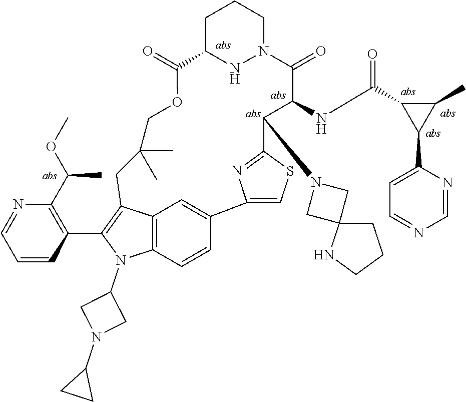 |
| A1185 | 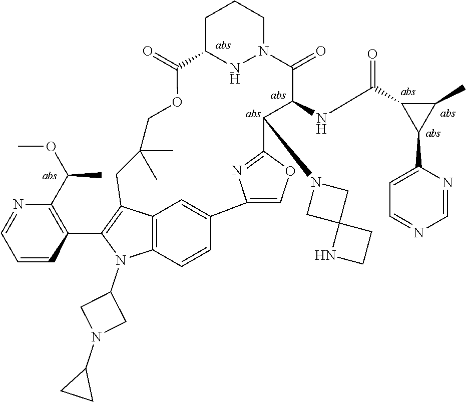 |
| A1186 | 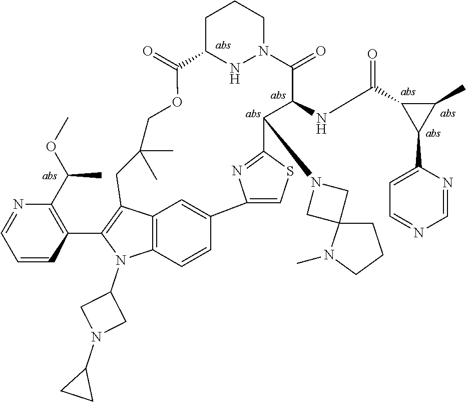 |
| A1187 | 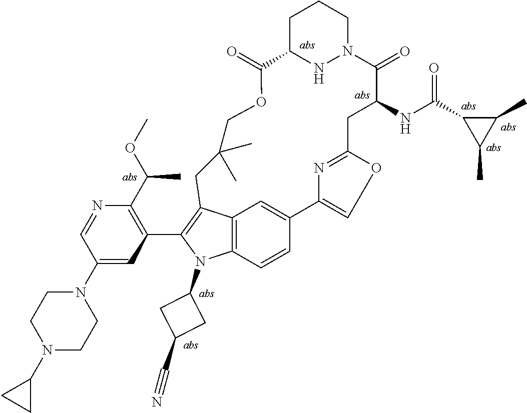 |
| A1188 | 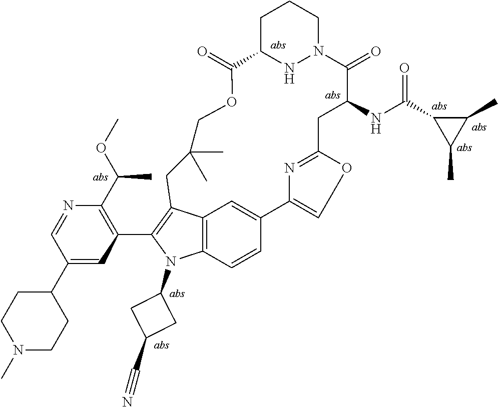 |
| A1189 | 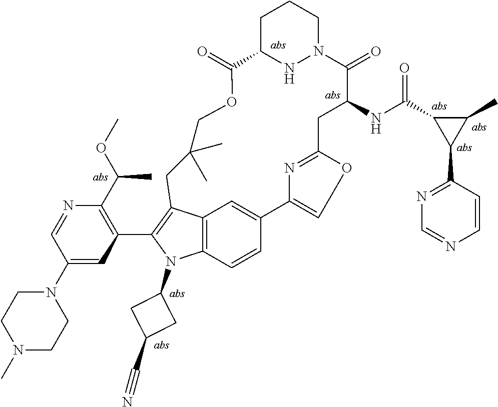 |
| A1190 | 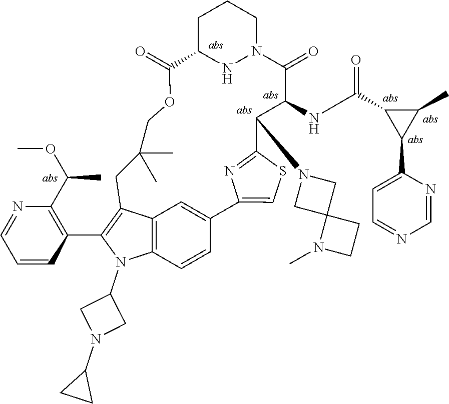 |
| A1191 | 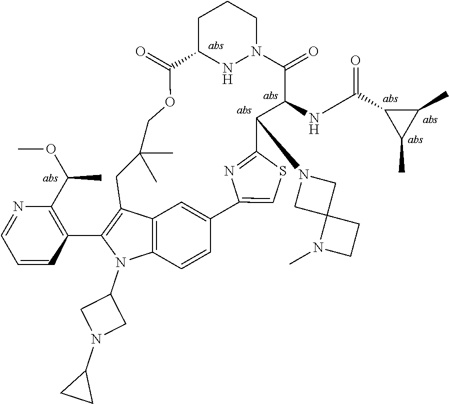 |
| A1192 | 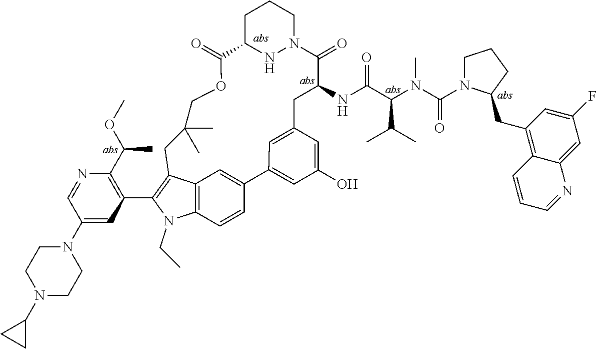 |
| A1193 | 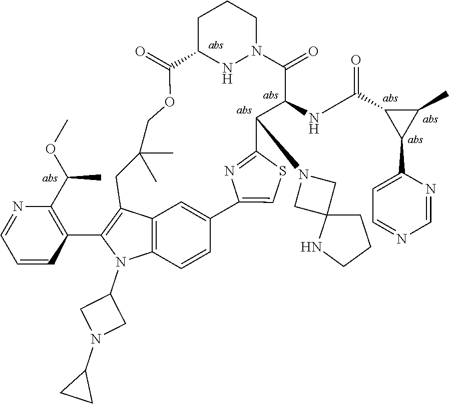 |
| A1194 | 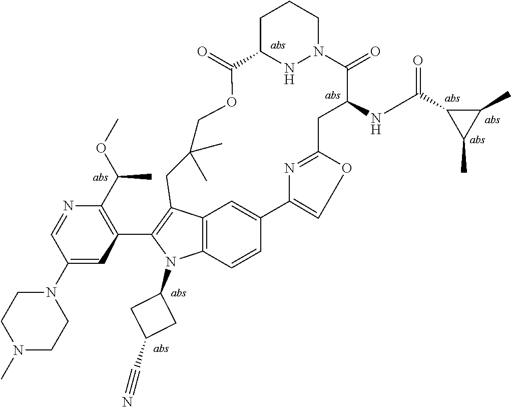 |
| A1195 | 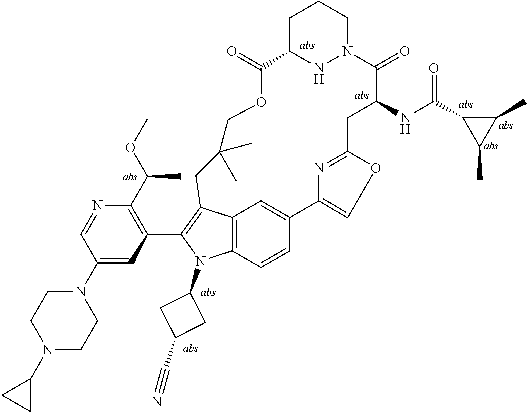 |
| A1196 | 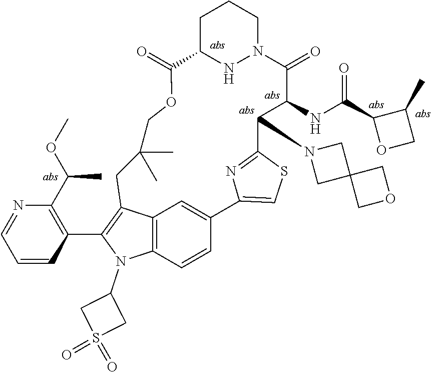 |
| A1197 | 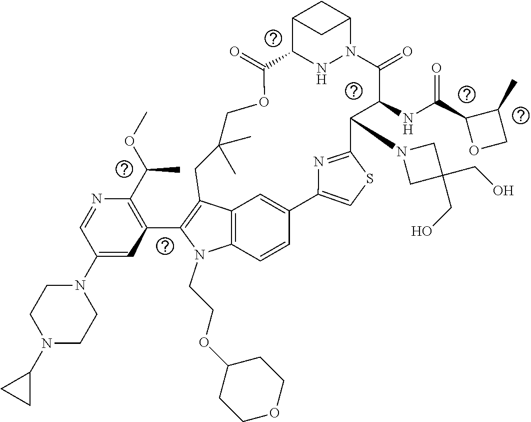 |
| A1198 | 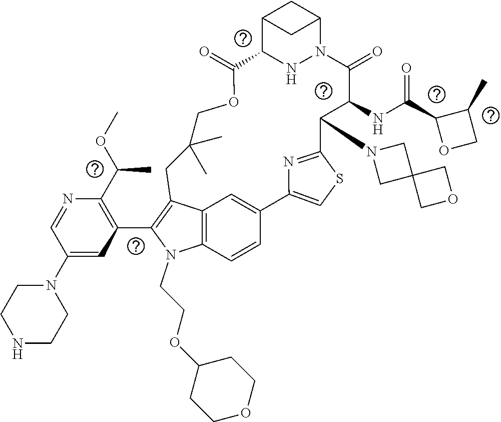 |
| A1199 | 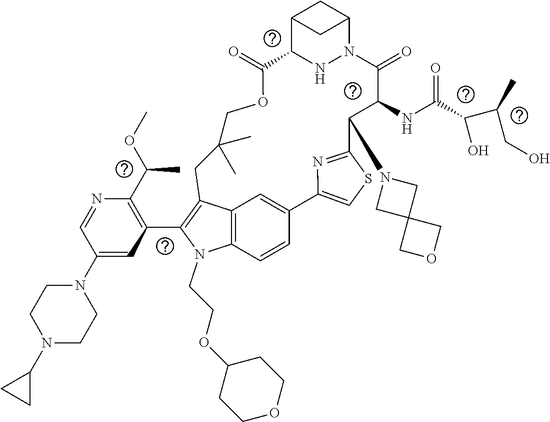 |
| A1200 | 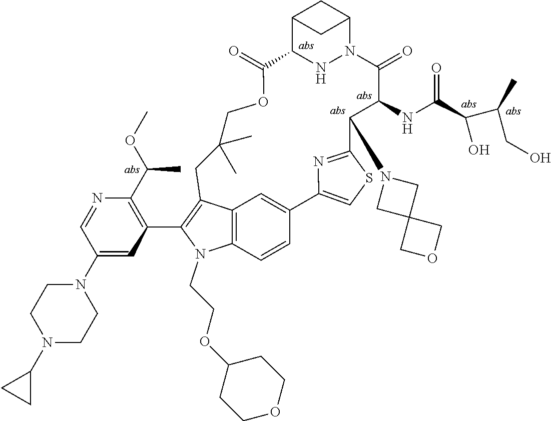 |

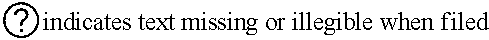

In some embodiments, a compound of the present invention is a compound selected from Table 3, or a pharmaceutically acceptable salt or stereoisomer thereof. In some embodiments, a compound of the present invention is a compound selected from Table 3, or a pharmaceutically acceptable salt or atropisomer thereof.

In some embodiments, a compound of the present invention is not a compound selected from Table 3. In some embodiments, a compound of the present invention is not a compound selected from Table 3, or a pharmaceutically acceptable salt or stereoisomer thereof. In some embodiments, a compound of the present invention is not a compound selected from Table 3, or a pharmaceutically acceptable salt or atropisomer thereof.

TABLE 3
Certain Compounds

| | |
|---|---|
| 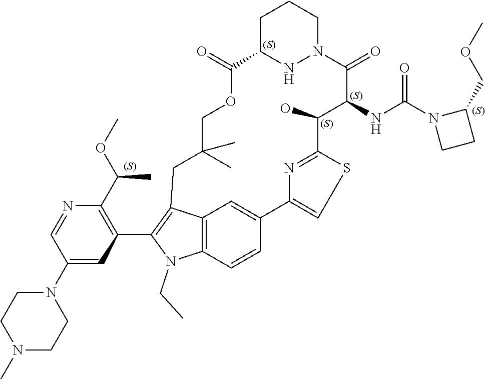 | B181 |
| 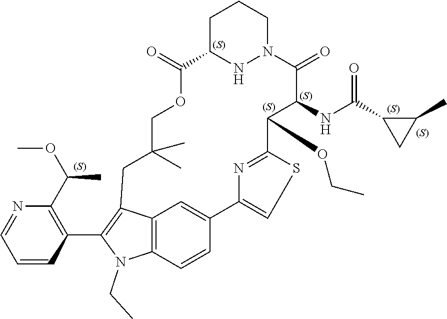 | B225 |
| 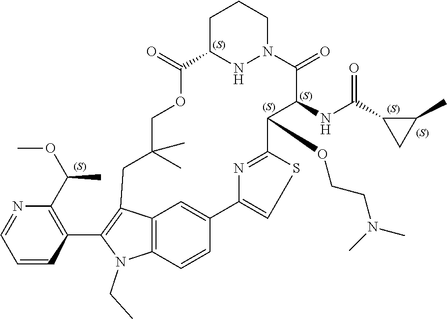 | B226 |
| 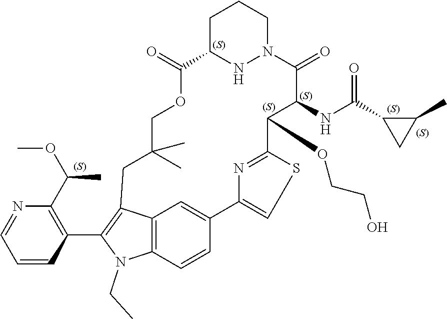 | B232 |
| 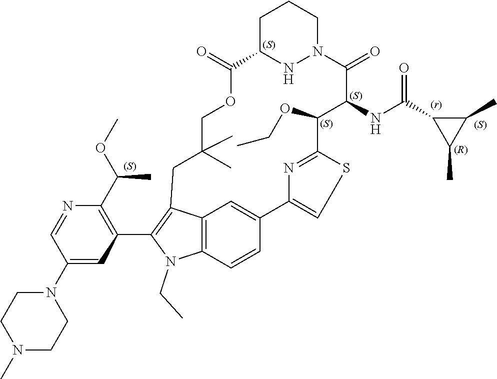 | B301 |
| 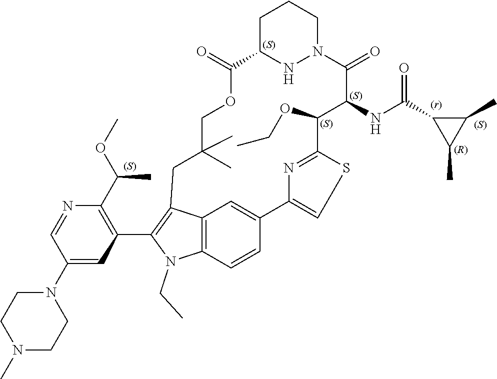 | B302 |
| 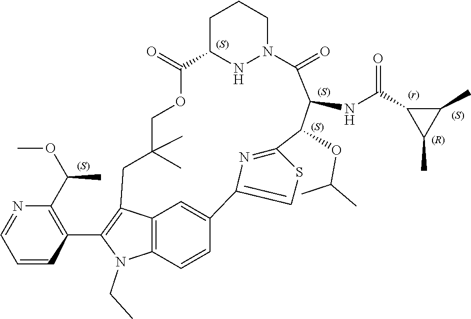 | B303 |
| 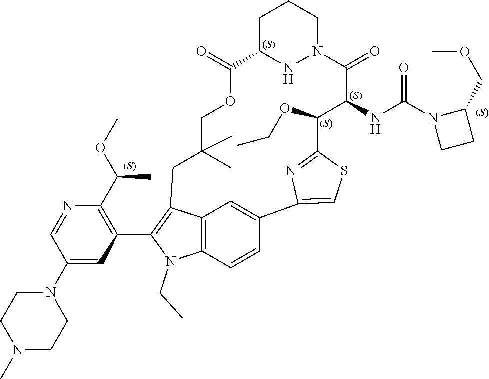 | B305 |
| 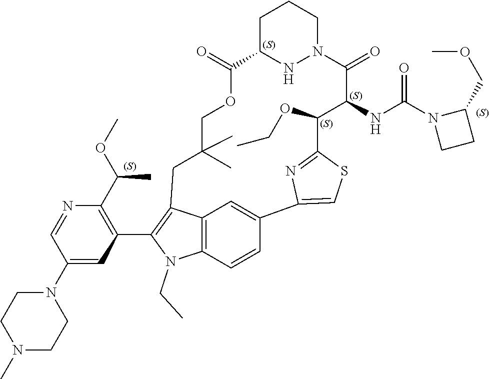 | B306 |
| 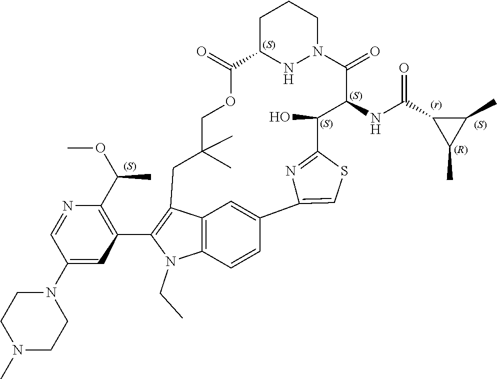 | B321 |
| 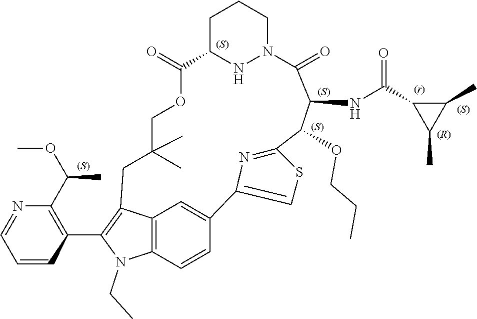 | B322 |
| 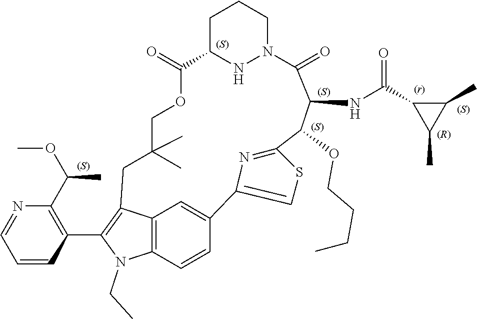 | B323 |
| 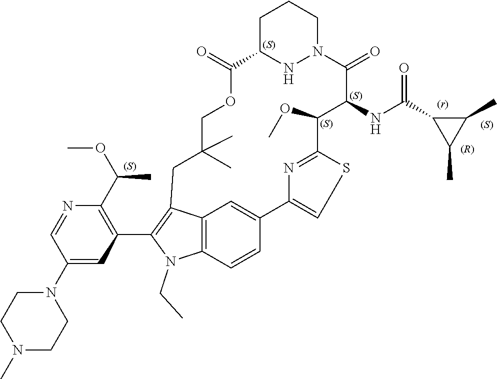 | B327 |
| 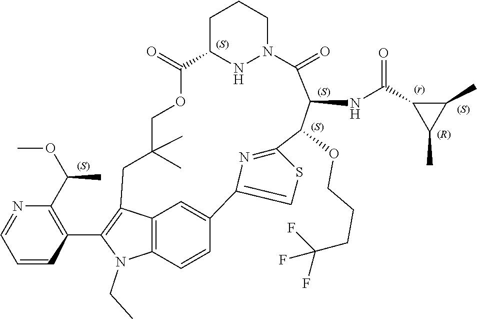 | B328 |
| 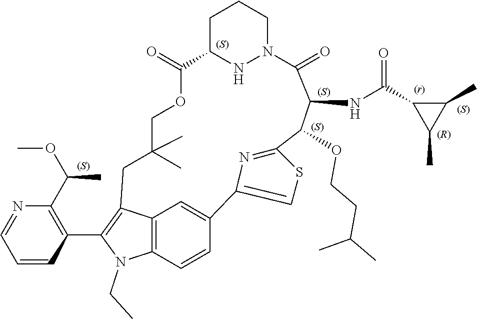 | B334 |
| 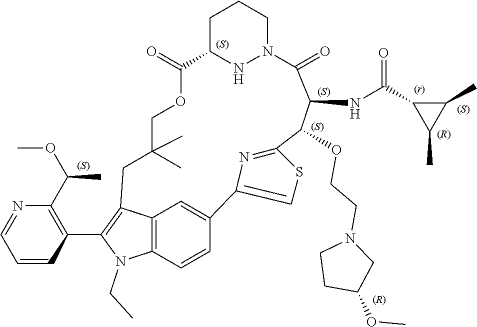 | B335 |
| 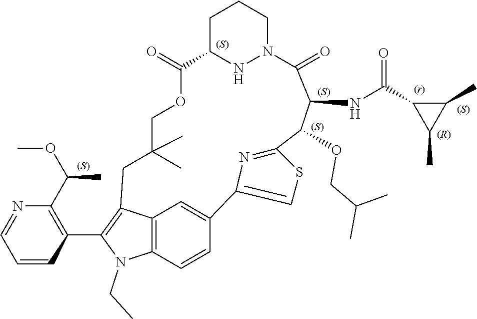 | B339 |
| 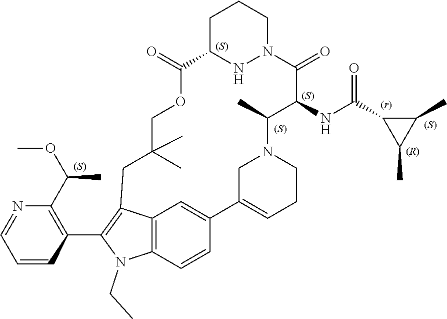 | B340 |
| 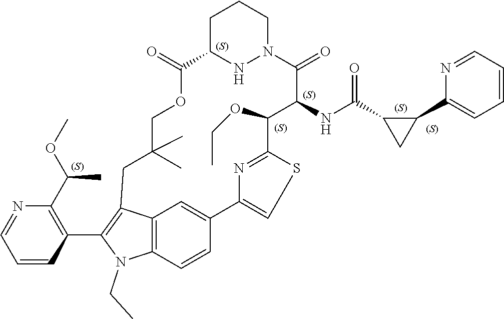 | B344 |
| 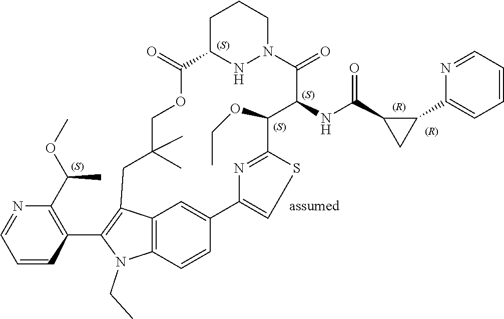 | B345 |
| 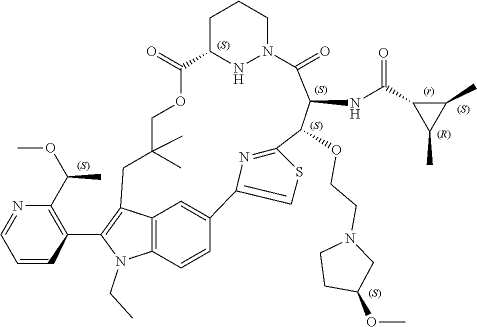 | B354 |
| 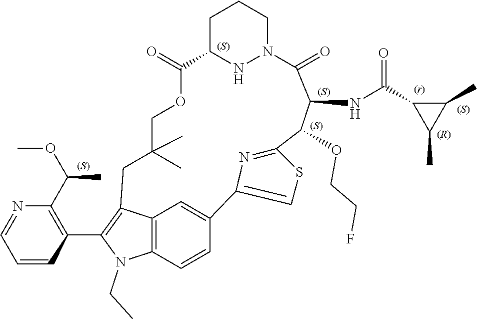 | B367 |
| 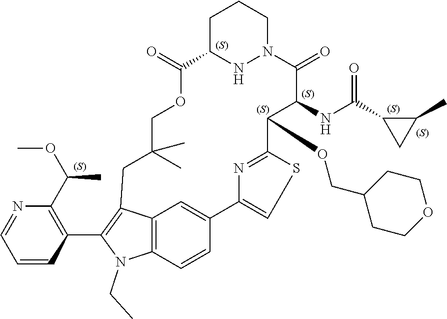 | B370 |
| 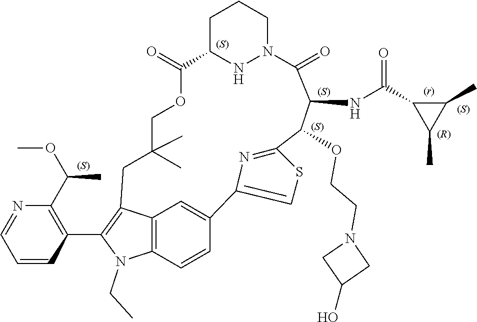 | B371 |
| 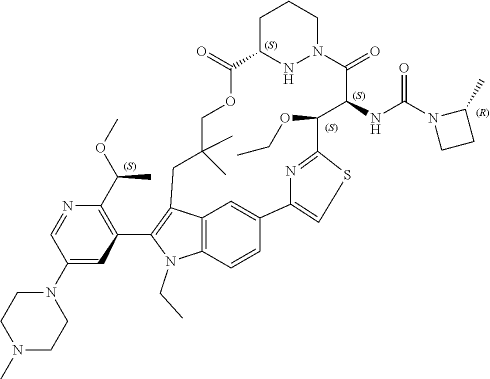 | B377 |
| 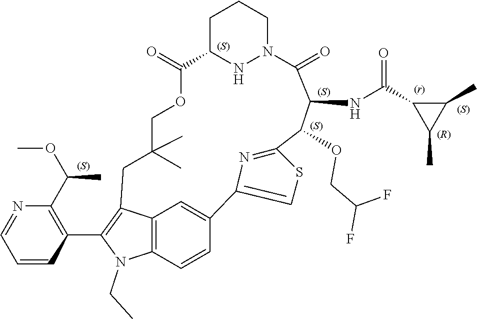 | B387 |
| 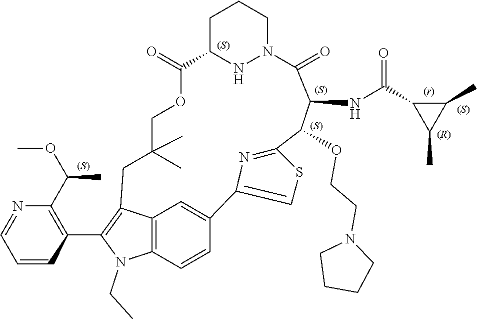 | B388 |
| 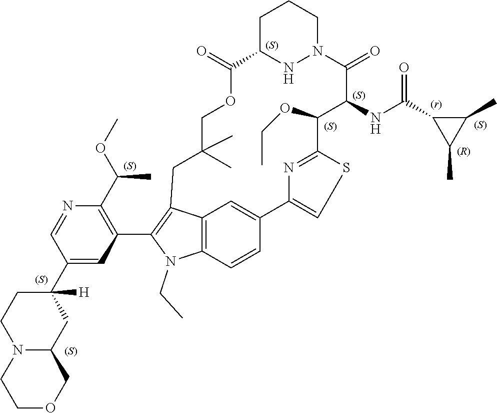 | B389 |
| 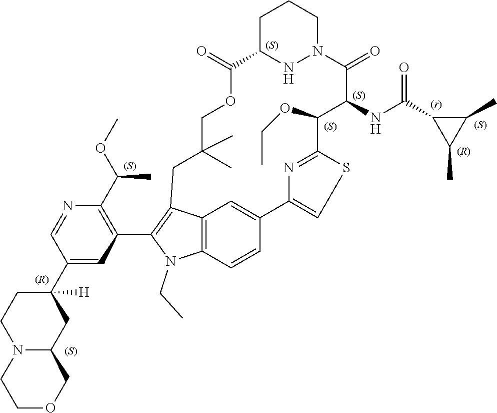 | B390 |
| 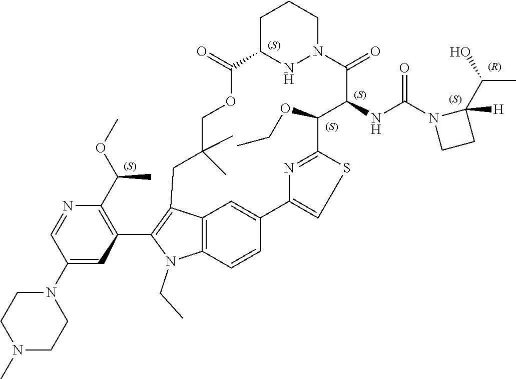 | B396 |
| 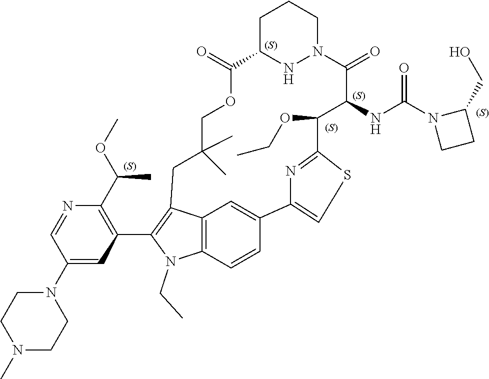 | B397 |
| 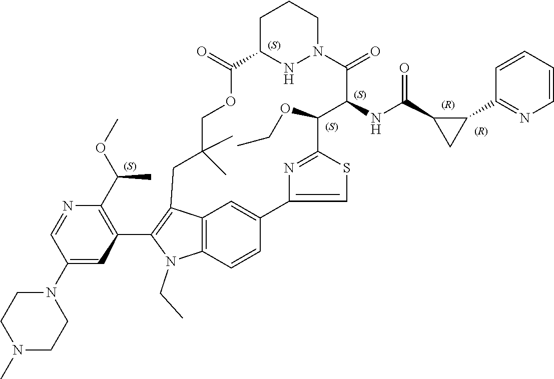 | B401 |
| 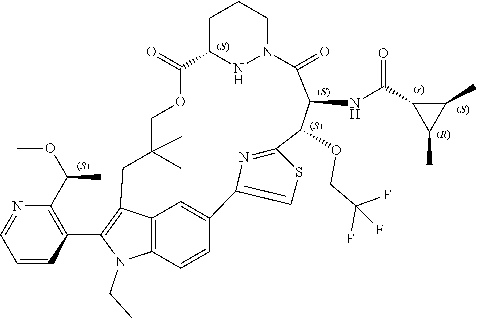 | B402 |
| 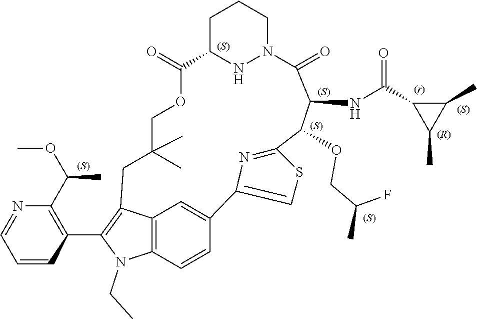 | B403 |
| 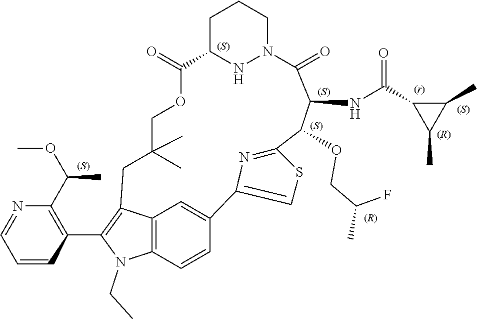 | B404 |
| 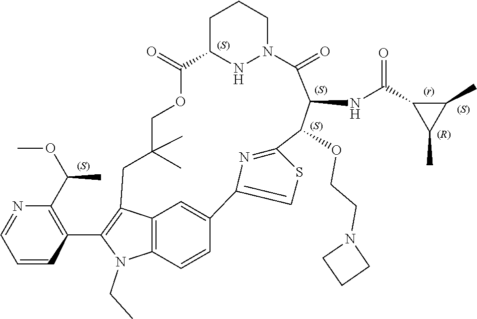 | B405 |
| 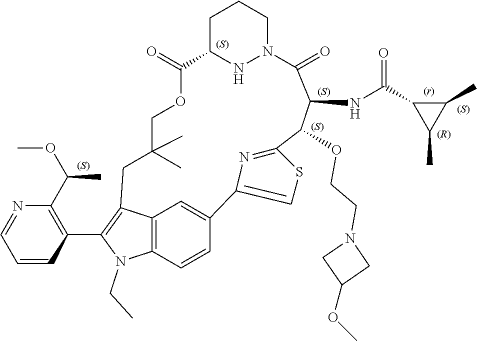 | B406 |
| 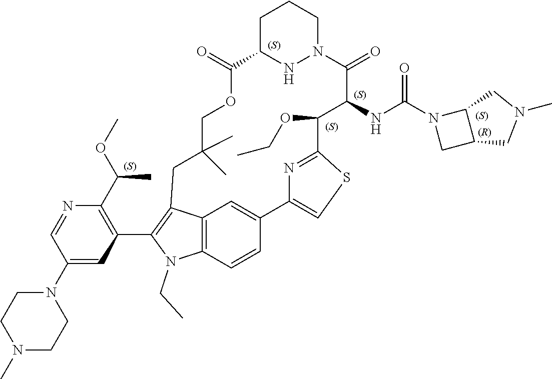 | B410 |
| 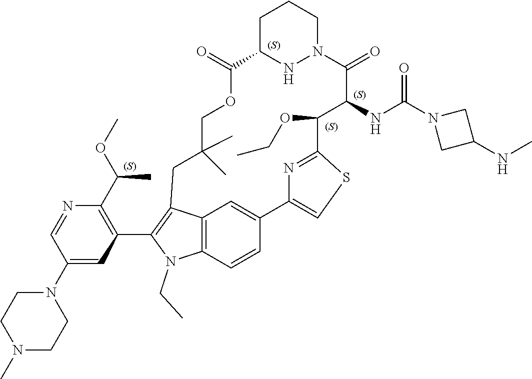 | B411 |
| 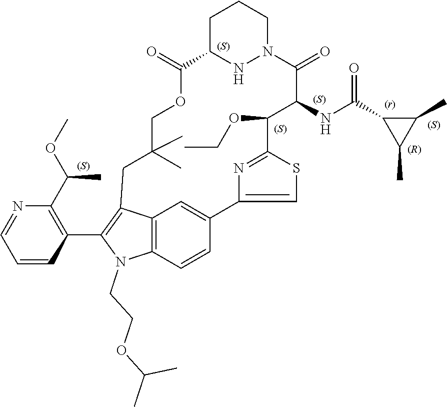 | B416 |
| 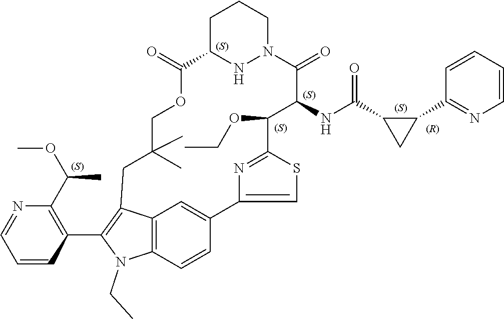 | B417 |
| 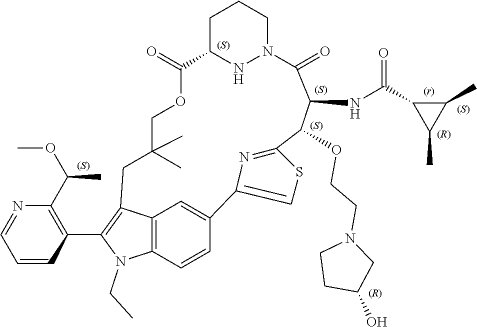 | B421 |
| 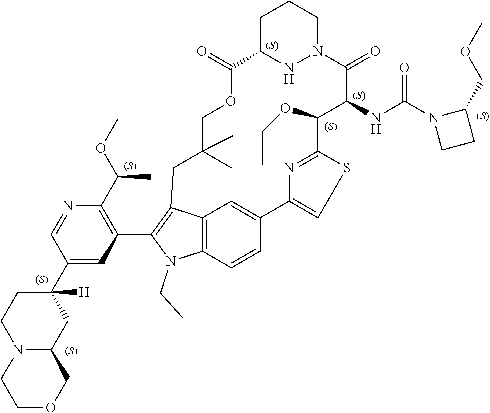 | B426 |
| 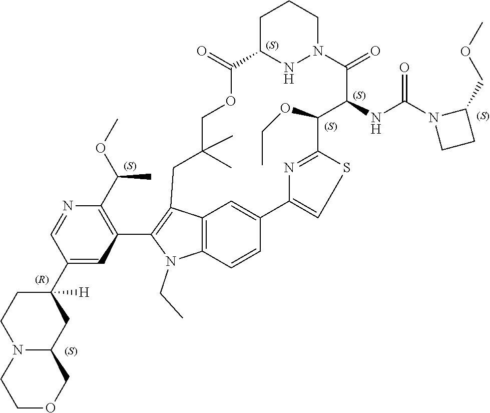 | B427 |
| 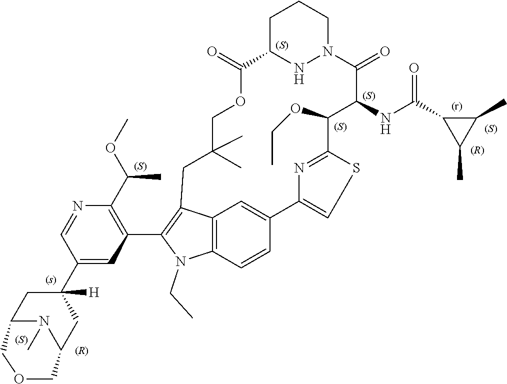 | B428 |
| 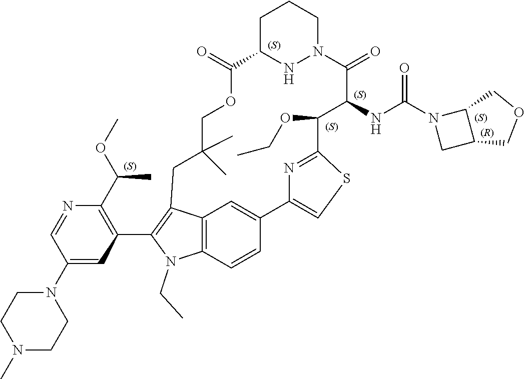 | B433 |
| 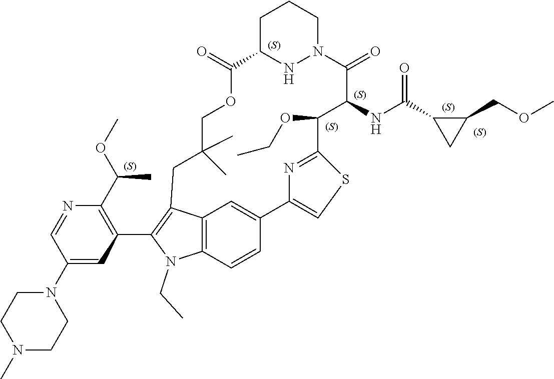 | B434 |
| 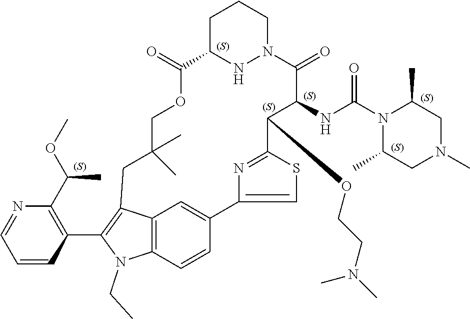 | B437 |
| 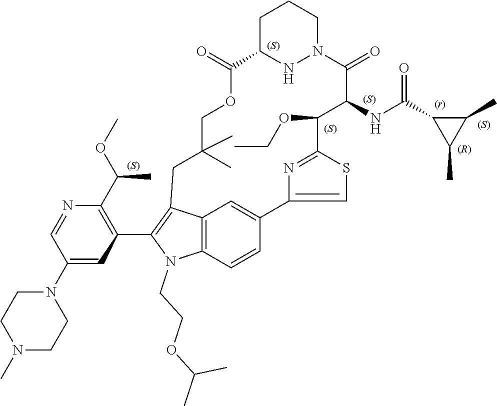 | B438 |
| 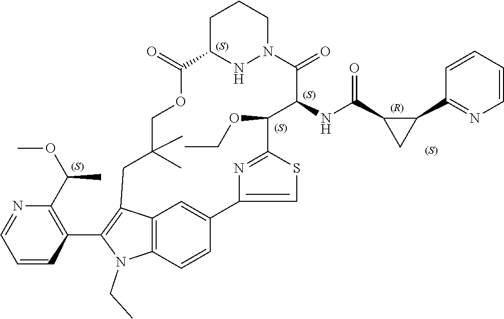 | B439 |
| 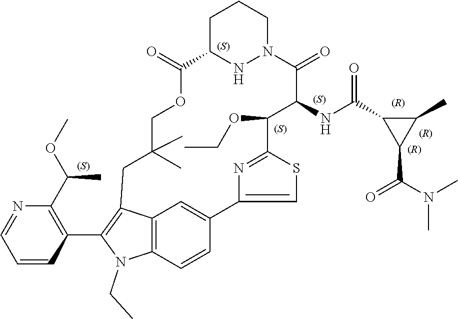 | B440 |
| 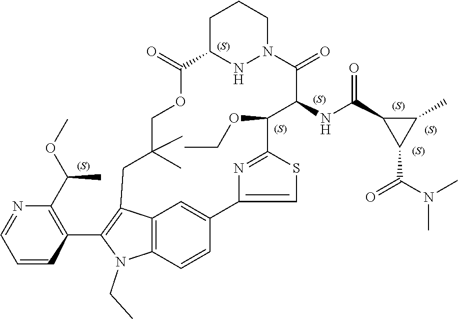 | B441 |
| 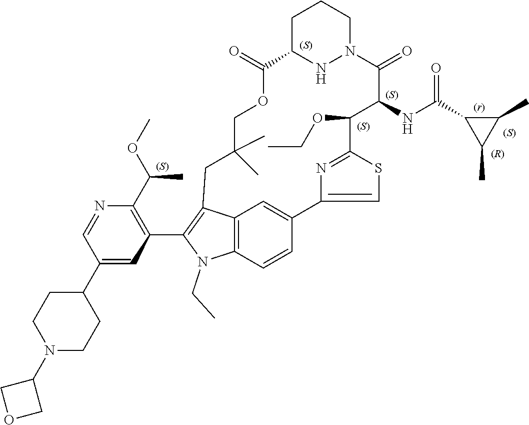 | B449 |
| 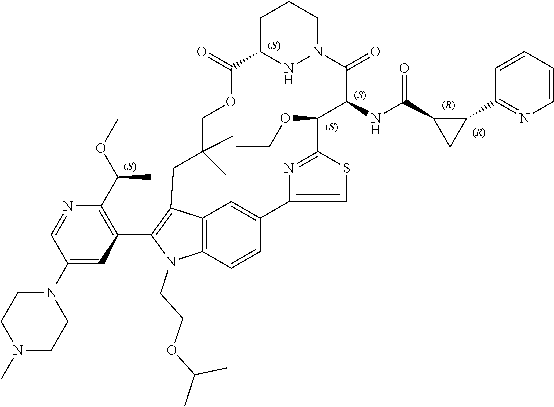 | B450 |
| 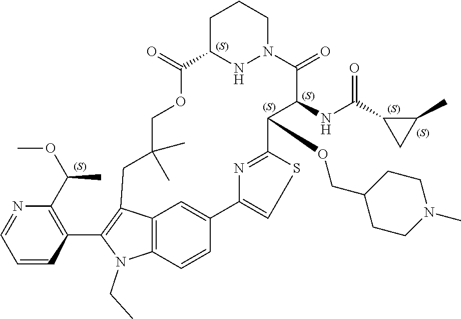 | B451 |
| 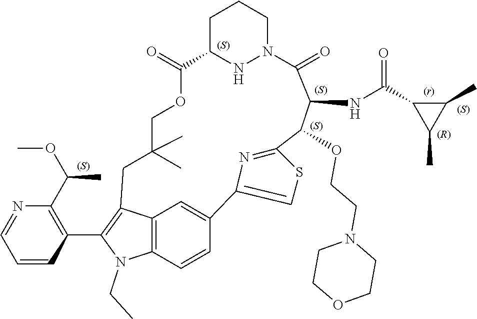 | B452 |
| 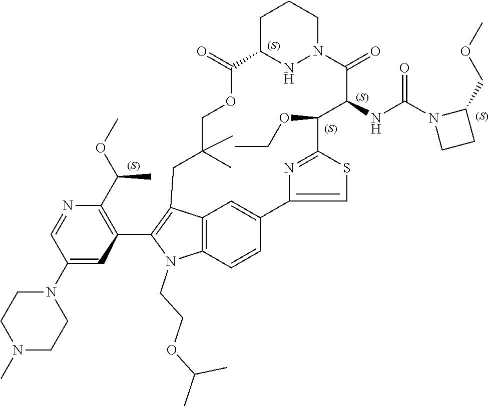 | B459 |
| 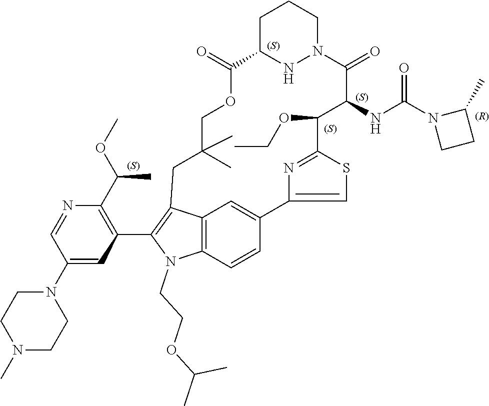 | B460 |
| 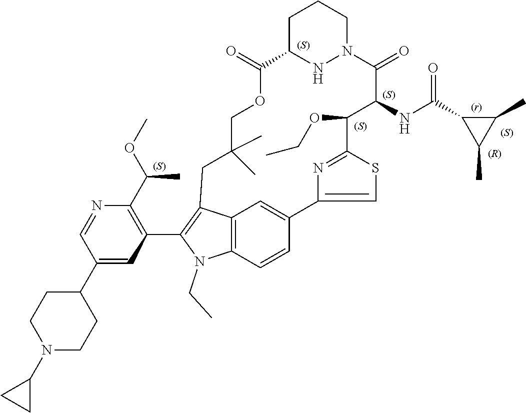 | B461 |
| 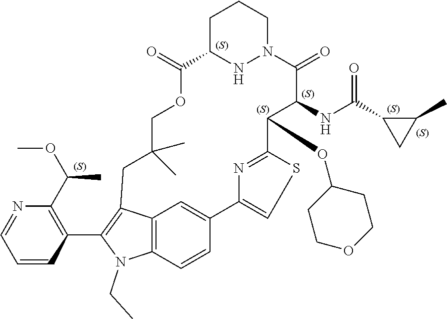 | B462 |
| 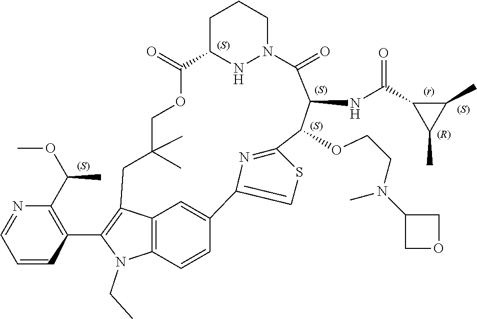 | B467 |
| 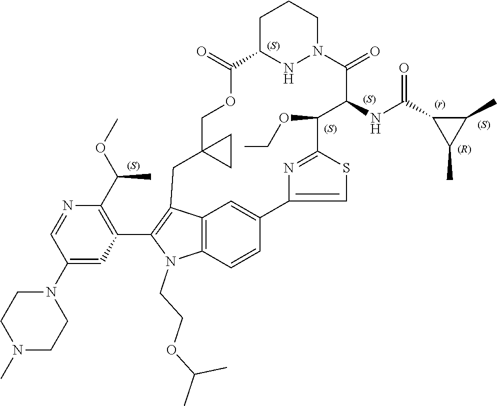 | B475 |
| 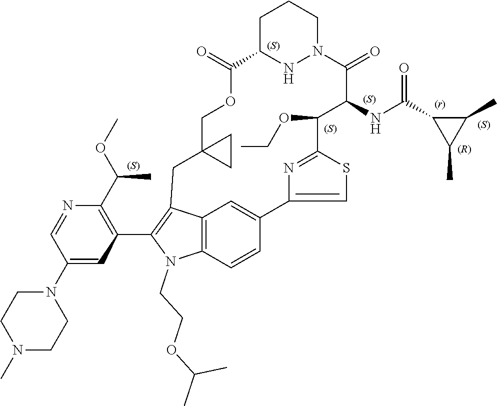 | B476 |
| 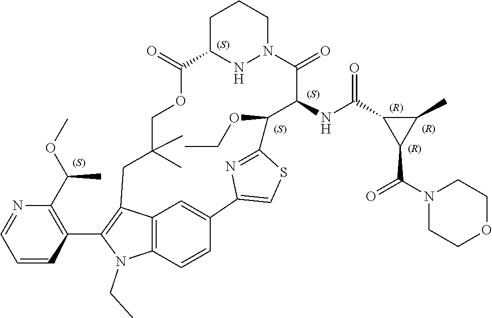 | B477 |
| 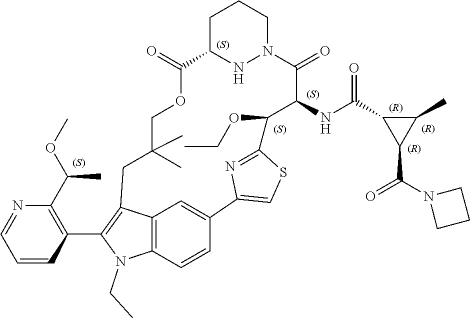 | B478 |
| 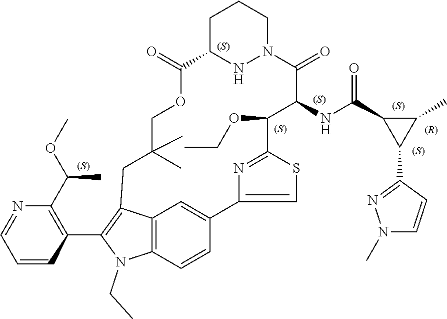 | B479 |
| 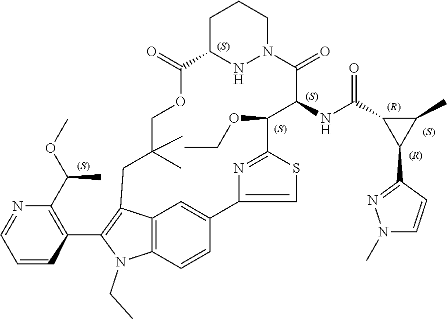 | B480 |
| 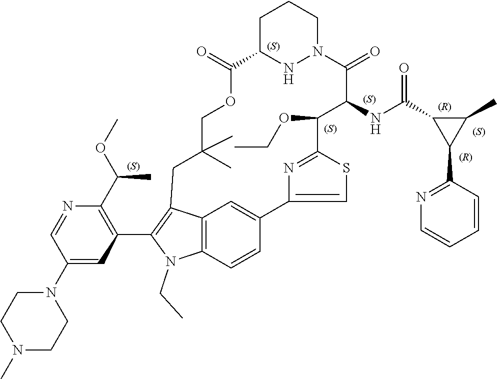 | B481 |
| 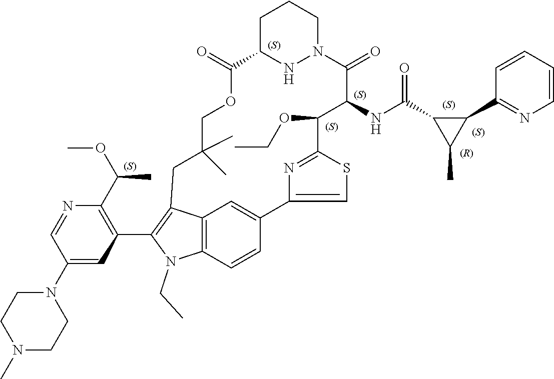 | B482 |
| 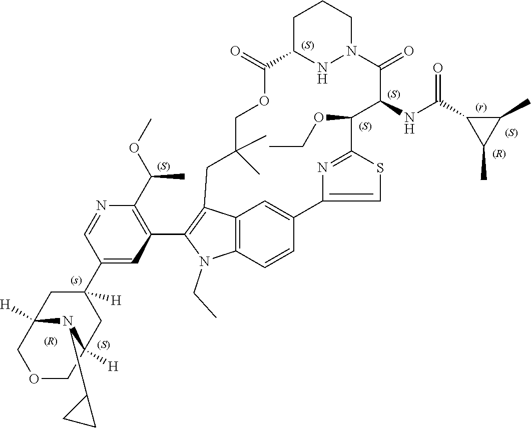 | B483 |
| 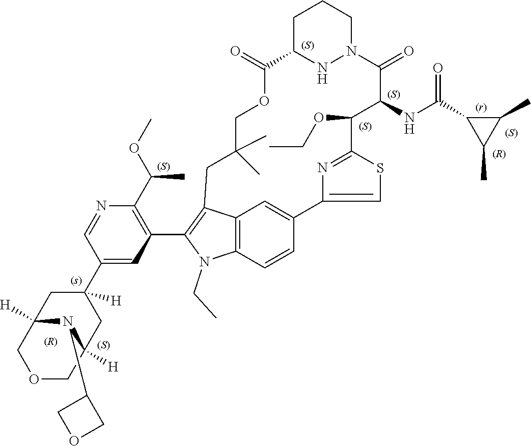 | B484 |
| 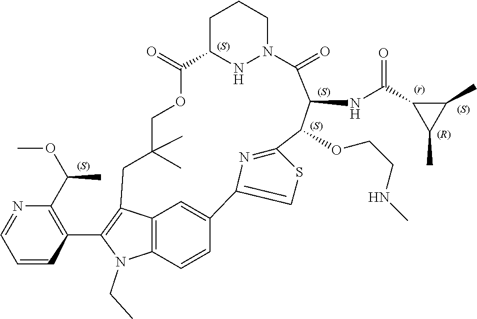 | B490 |
| 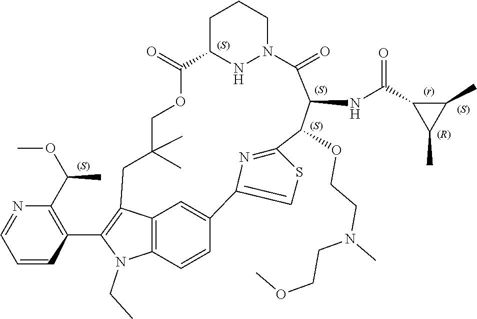 | B493 |
| 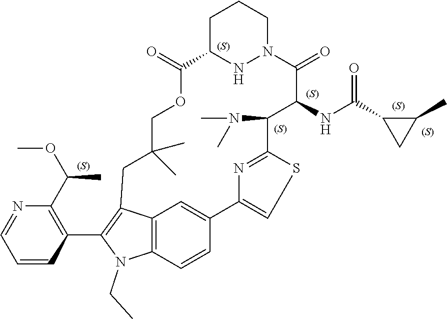 | B496 |
| 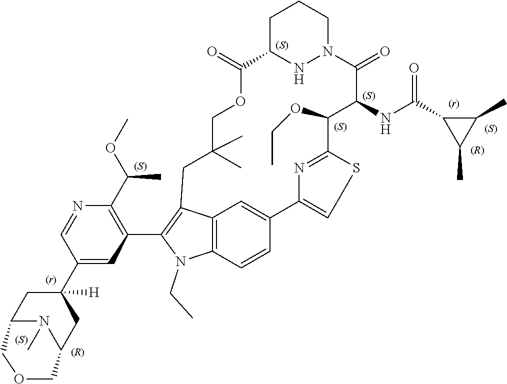 | B497 |
| 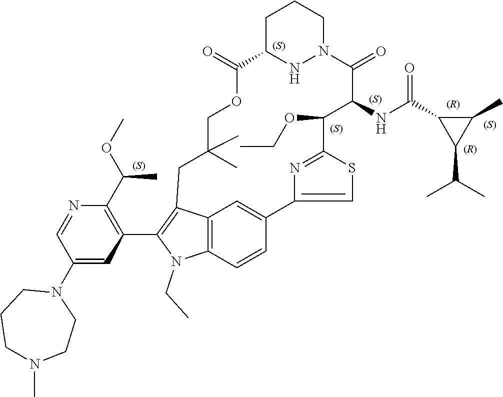 | B500 |
| 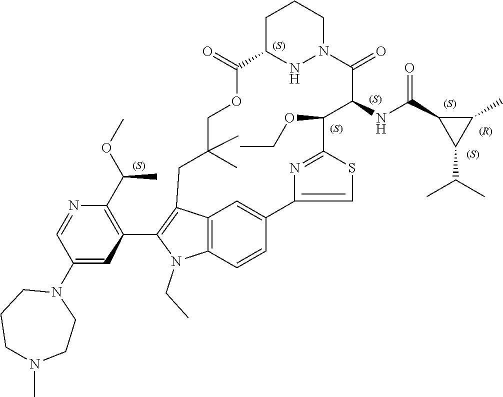 | B501 |
| 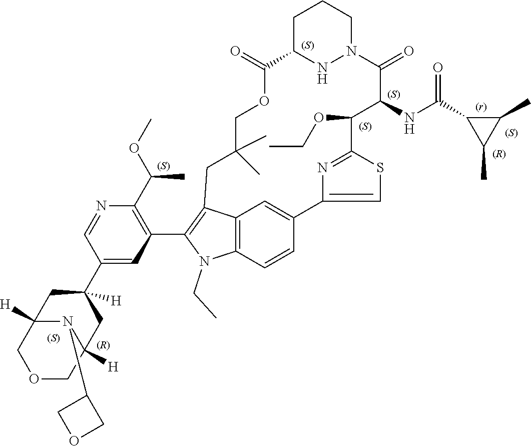 | B502 |
| 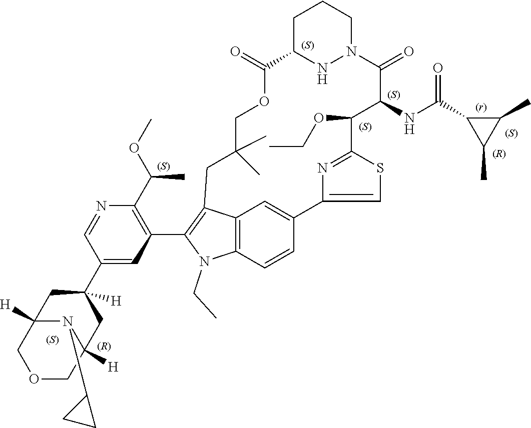 | B503 |
| 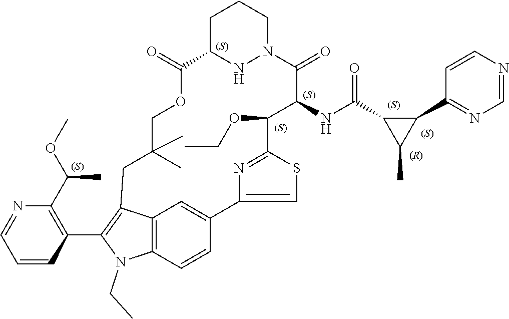 | B504 |
| 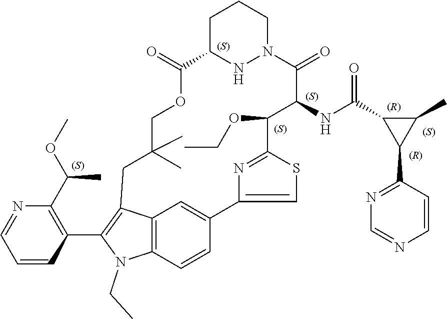 | B505 |
| 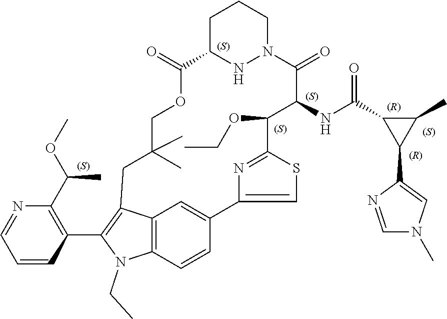 | B506 |
| 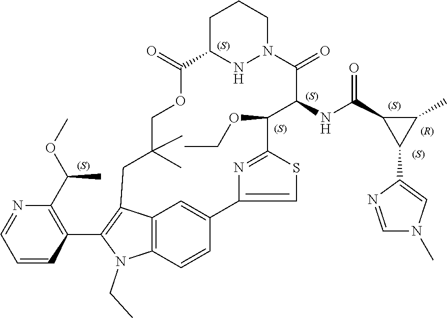 | B507 |
| 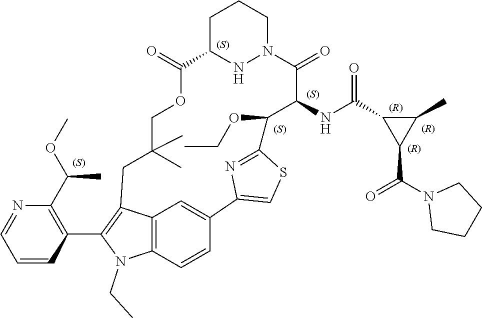 | B508 |
| 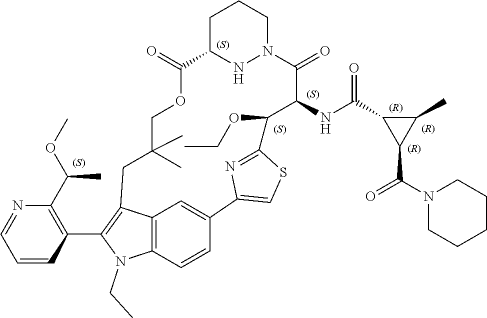 | B509 |
| 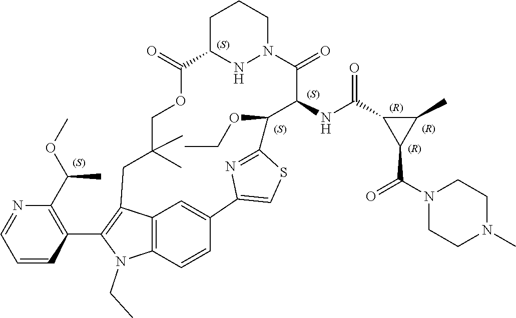 | B510 |
| 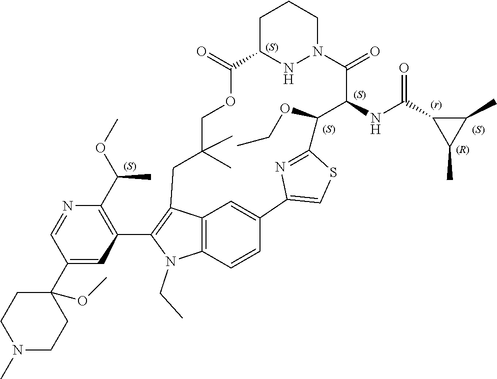 | B511 |
| 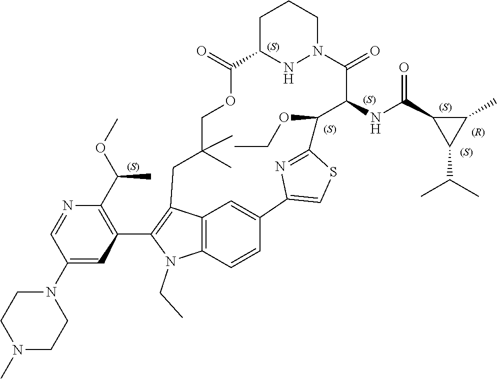 | B512 |
| 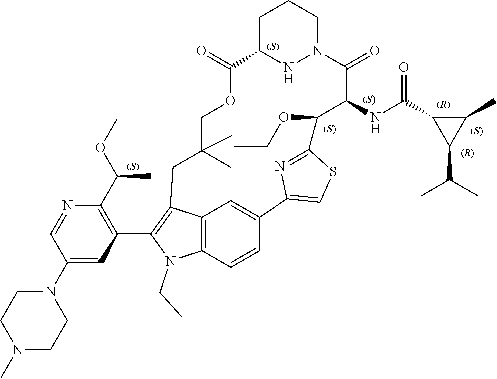 | B513 |
| 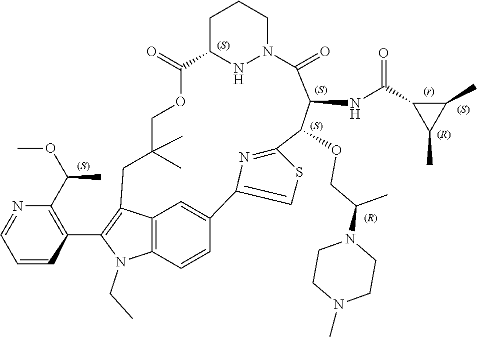 | B515 |
| 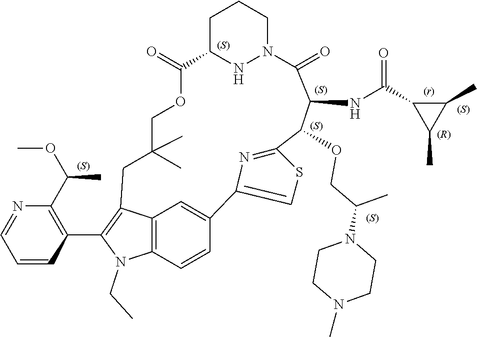 | B516 |
| 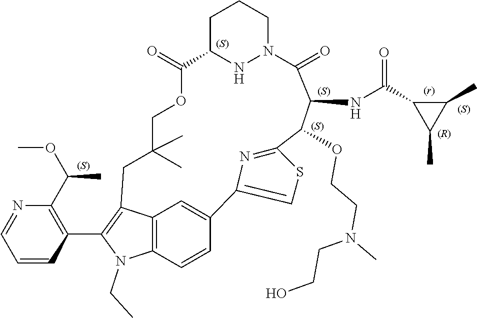 | B517 |
| 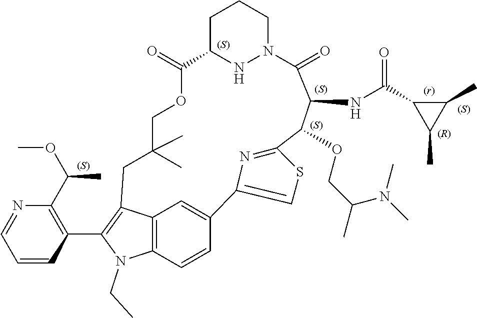 | B518 |
| 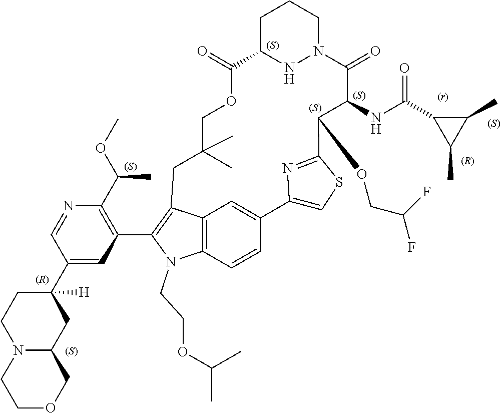 | B520 |
| 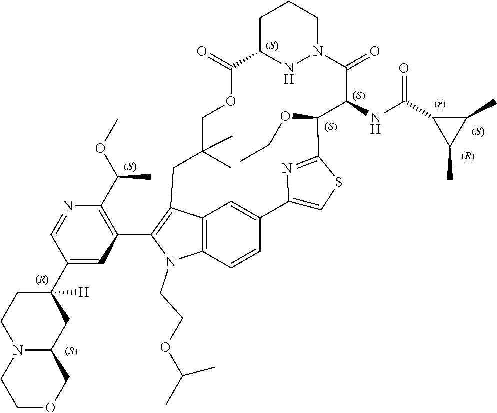 | B521 |
| 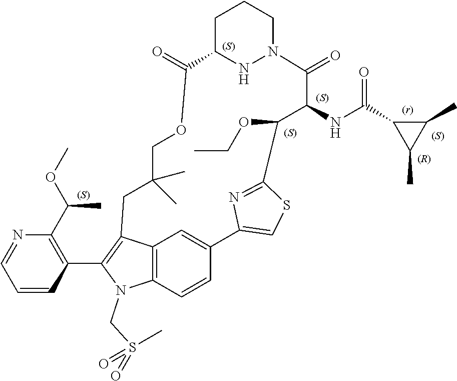 | B522 |
| 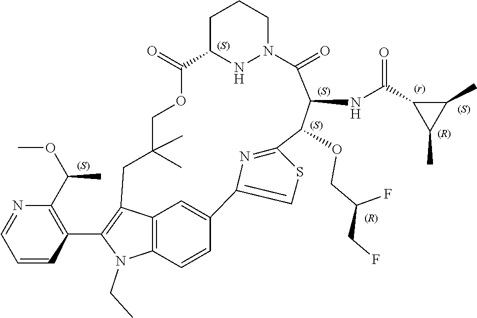 | B523 |
| 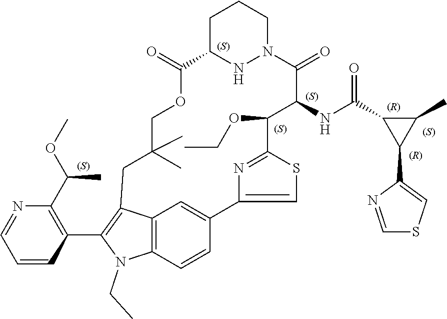 | B531 |
| 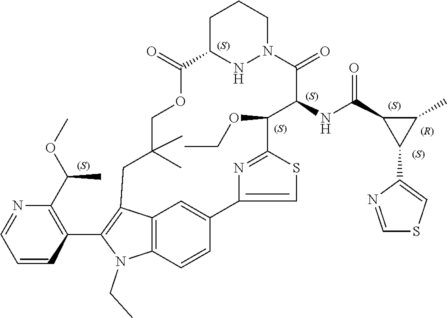 | B532 |
| 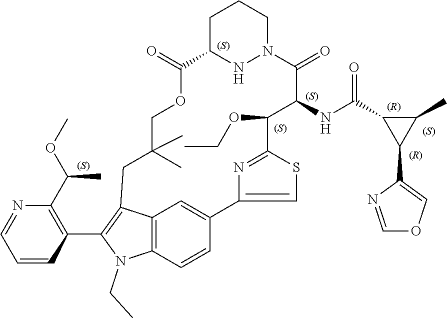 | B533 |
| 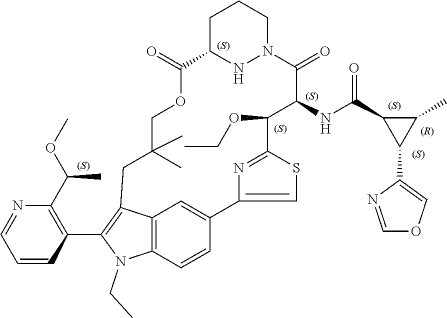 | B534 |
| 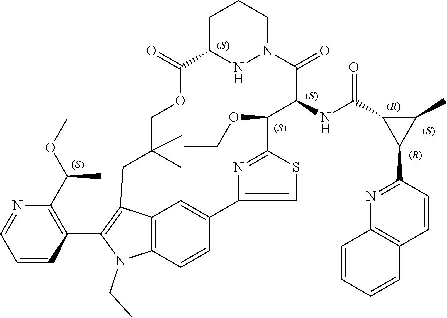 | B535 |
| 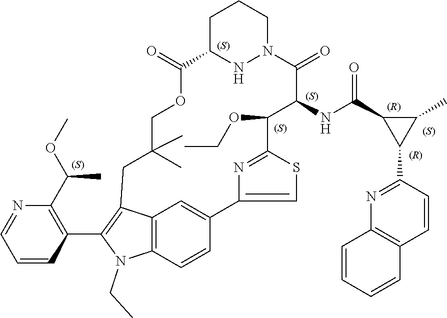 | B536 |
| 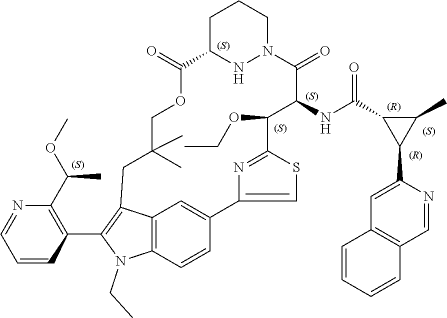 | B537 |
| 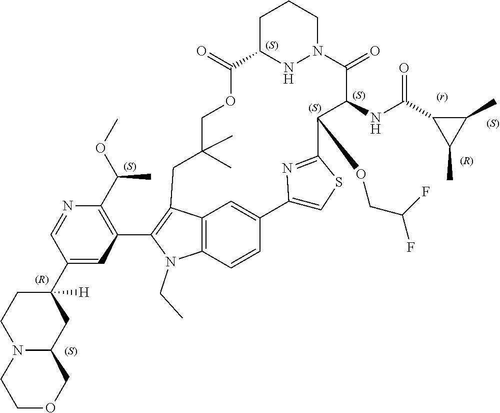 | B538 |
| 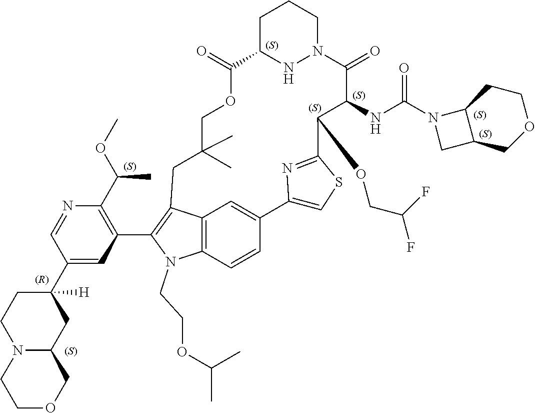 | B539 |
| 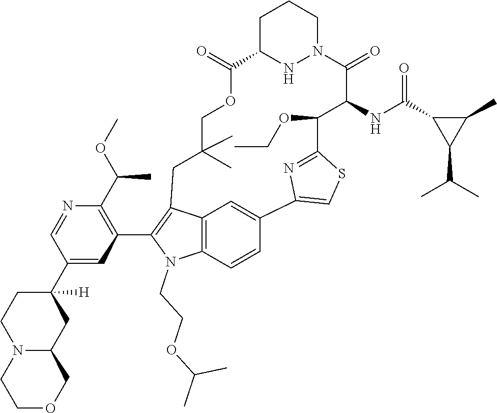 | B540 |
| 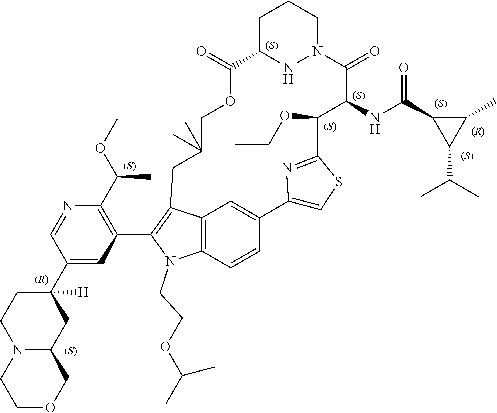 | B541 |
| 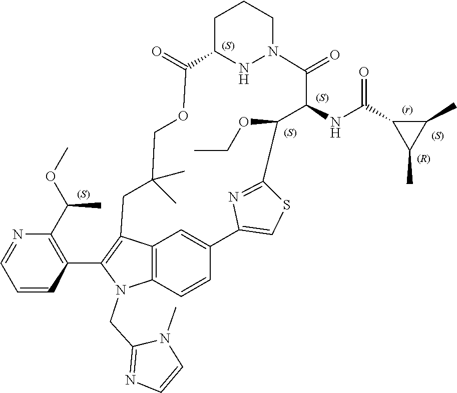 | B542 |
| 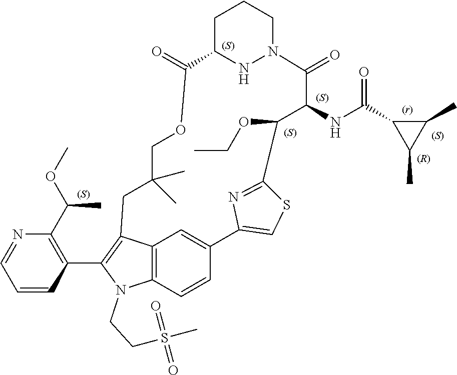 | B543 |
| 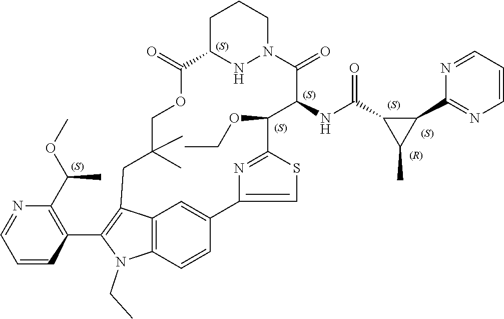 | B546 |
| 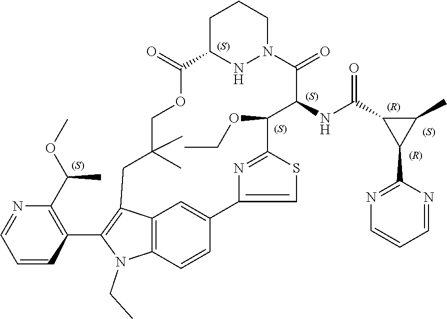 | B547 |
| 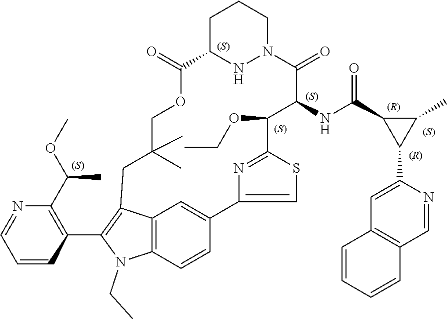 | B548 |
| 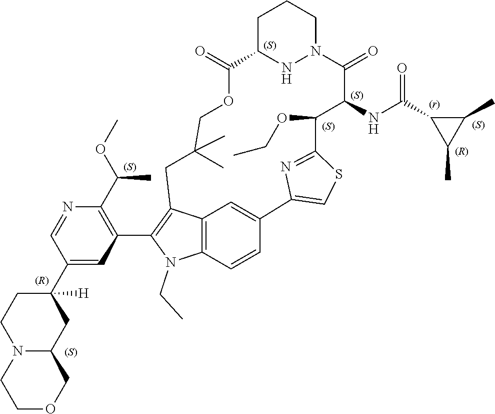 | B549 |
| 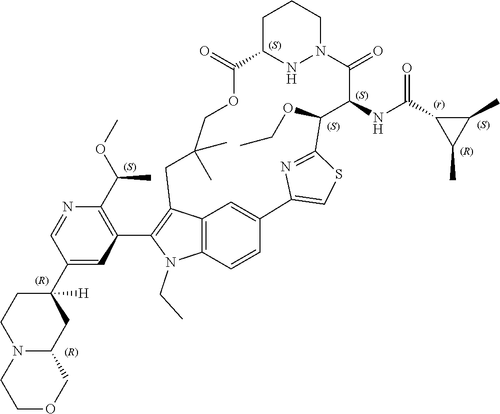 | B550 |
| 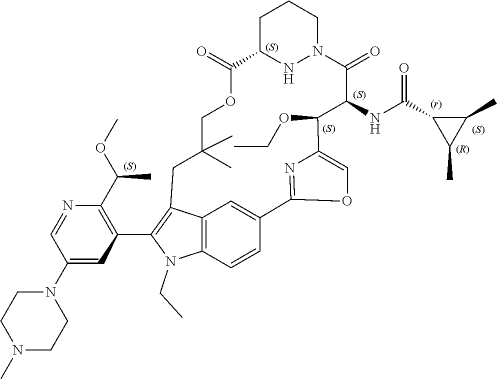 | B551 |
| 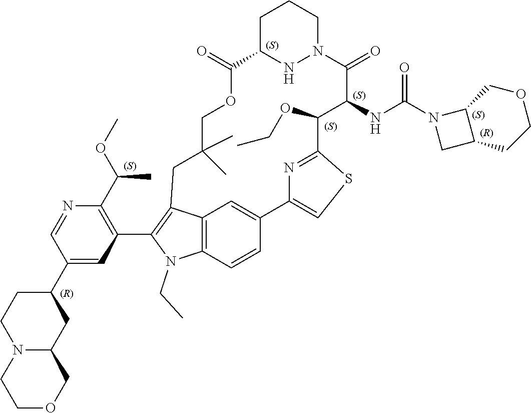 | B555 |
| 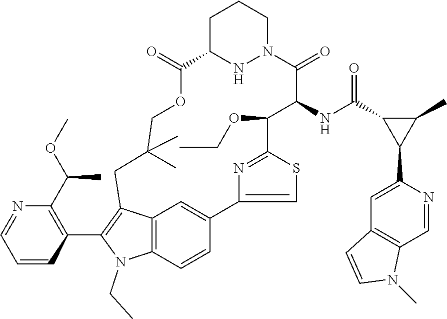 | B558 |
| 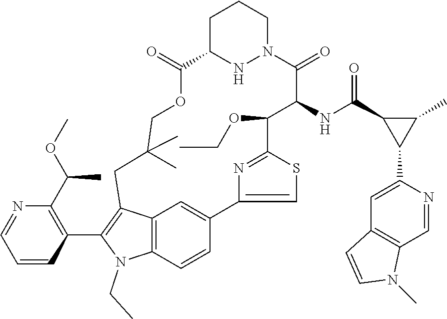 | B559 |
| 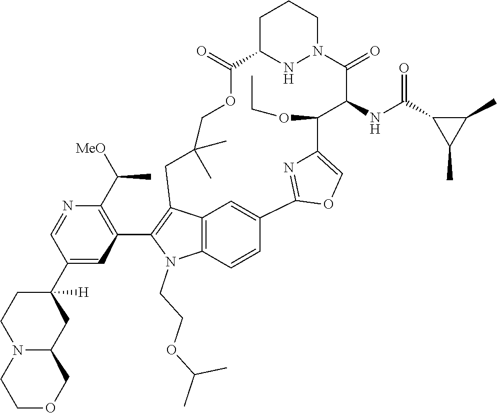 | B560 |
| 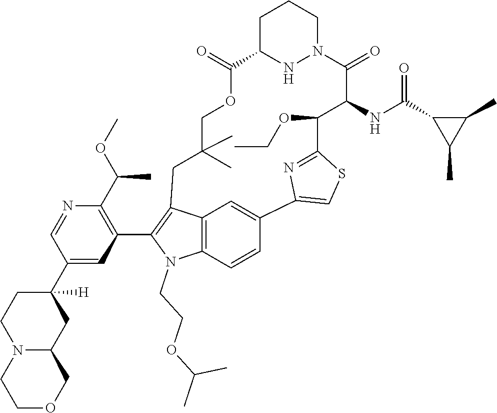 | B561 |
| 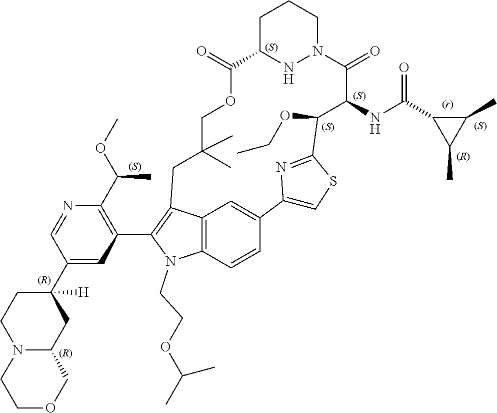 | B562 |
| 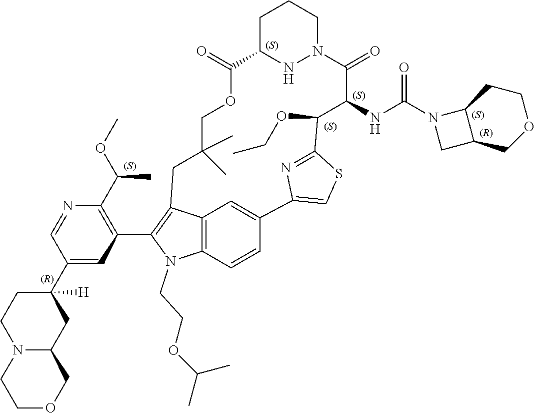 | B563 |
| 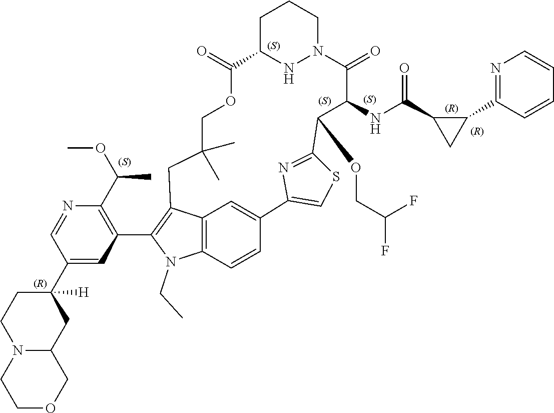 | B564 |
| 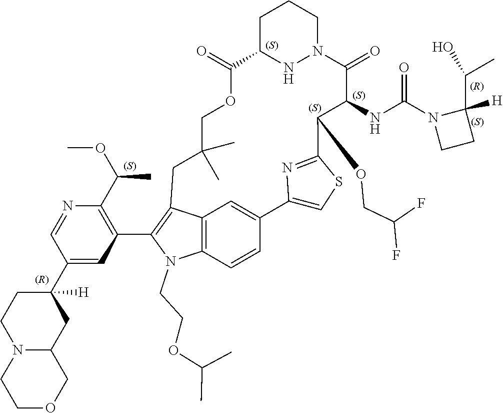 | B565 |
| 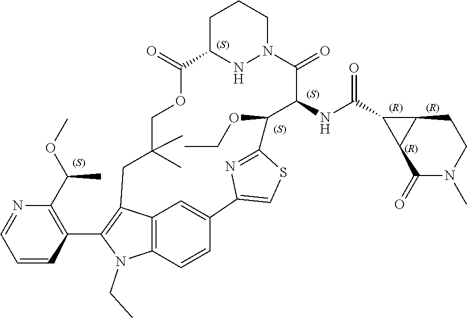 | B566 |
| 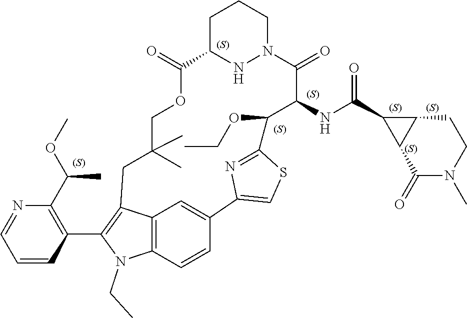 | B567 |
| 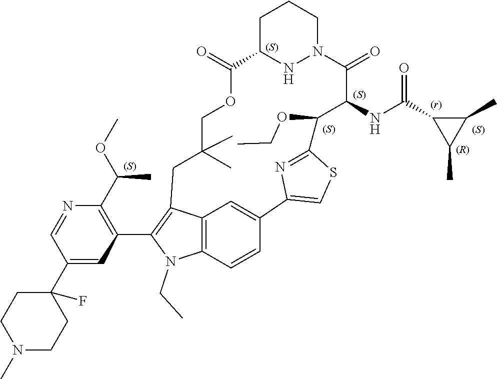 | B568 |
| 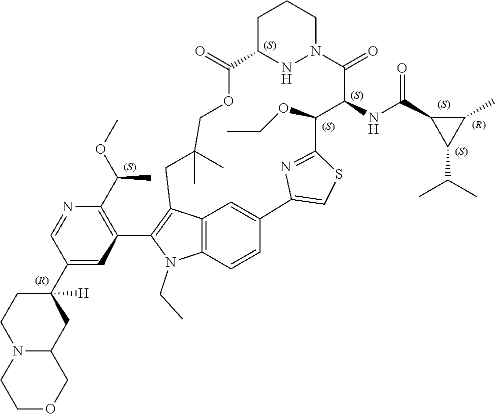 | B569 |
| 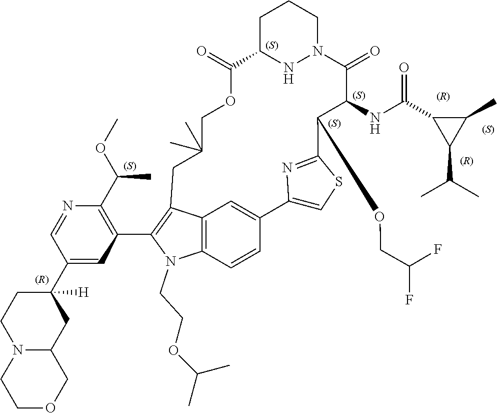 | B570 |
| 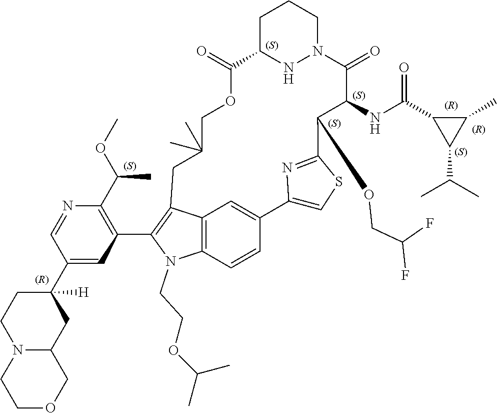 | B571 |
| 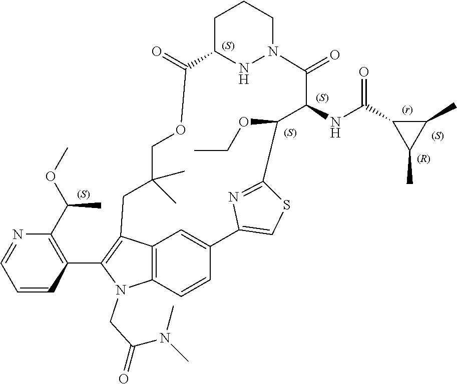 | B572 |
| 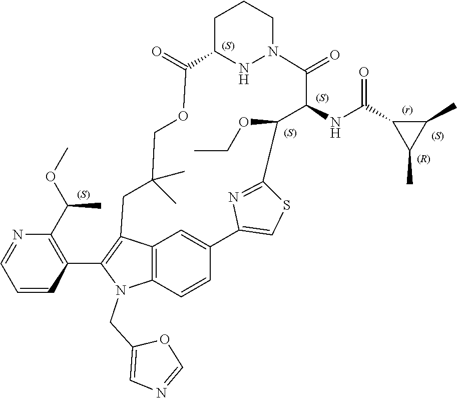 | B573 |
| 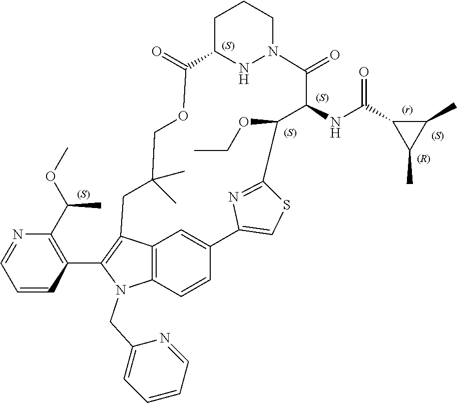 | B574 |
| 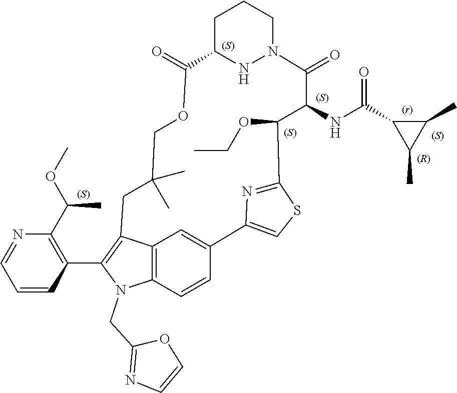 | B576 |
| 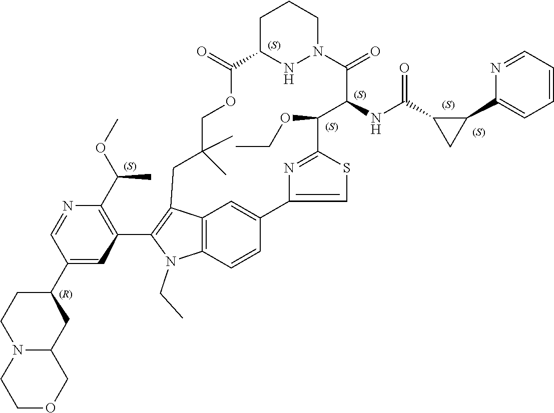 | B577 |
| 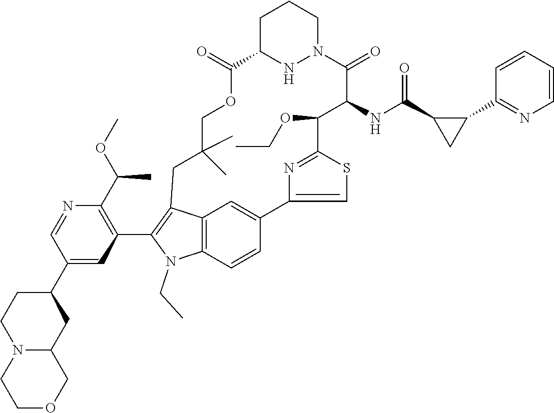 | B578 |
| 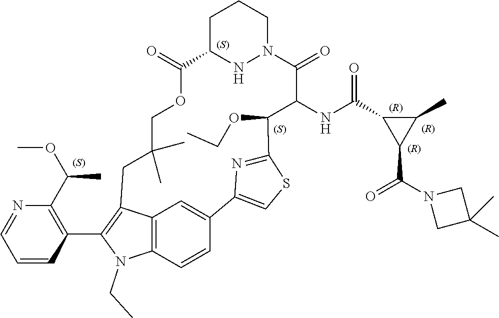 | B579 |
| 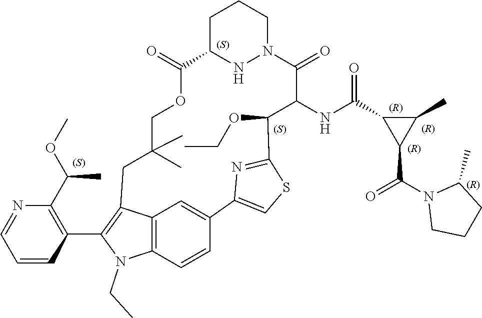 | B580 |
| 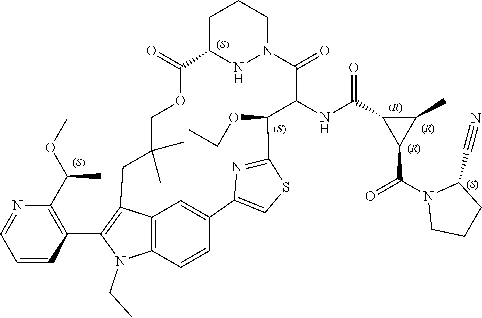 | B581 |
| 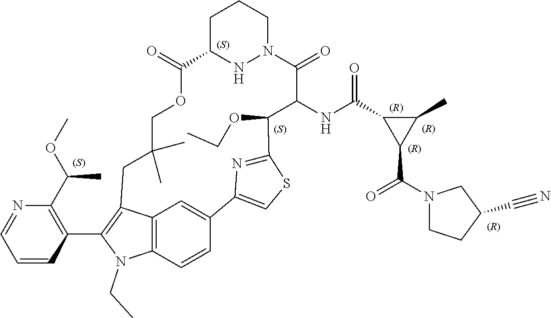 | B582 |
| 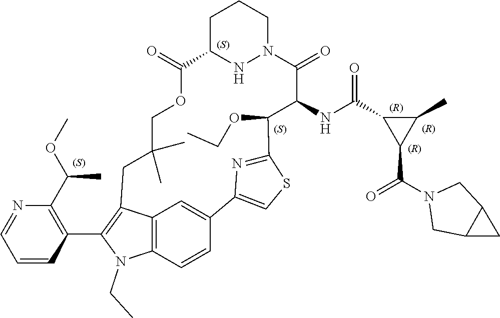 | B583 |
| 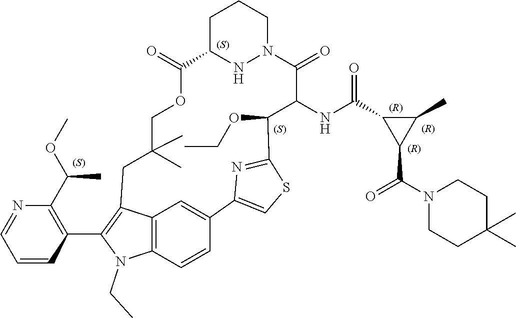 | B584 |
| 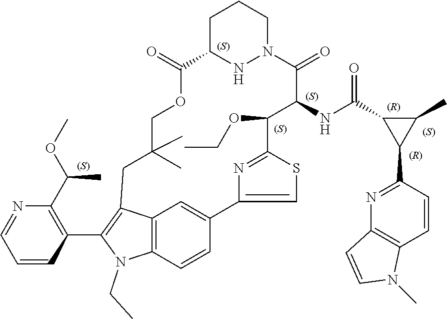 | B586 |
| 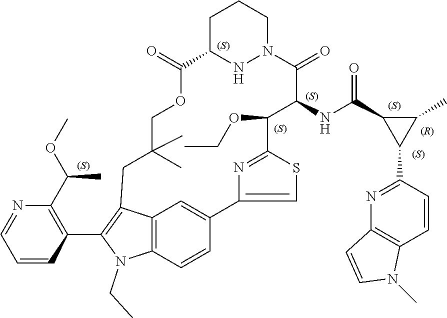 | B587 |
| 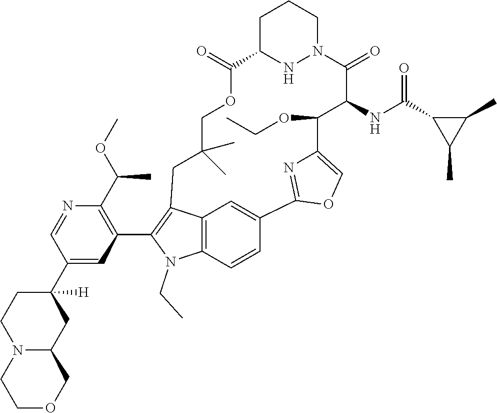 | B588 |
| 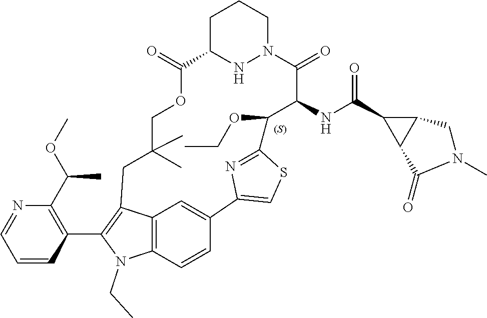 | B589 |
| 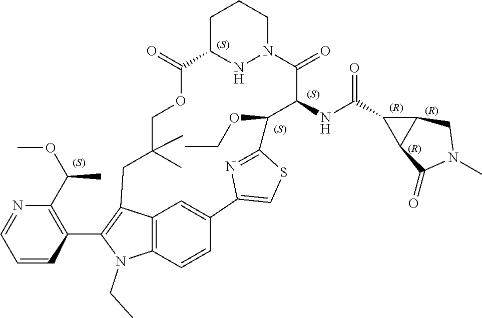 | B590 |
| 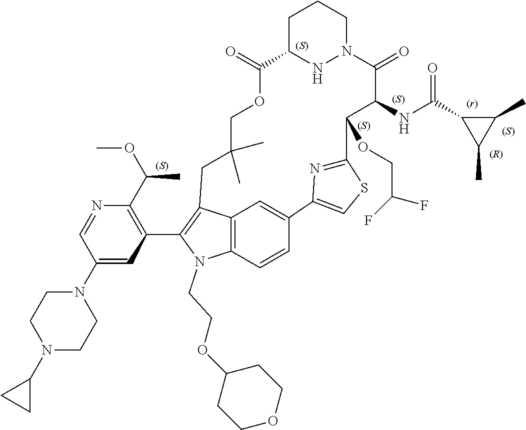 | B591 |
| 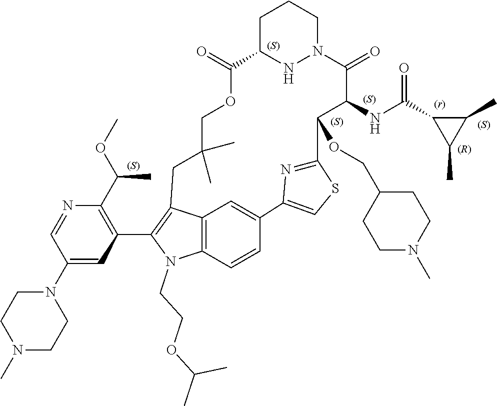 | B592 |
| 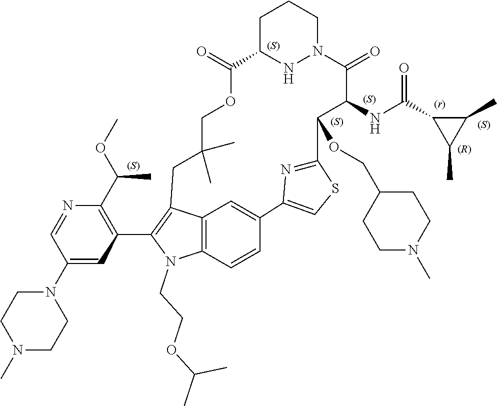 | B593 |
| 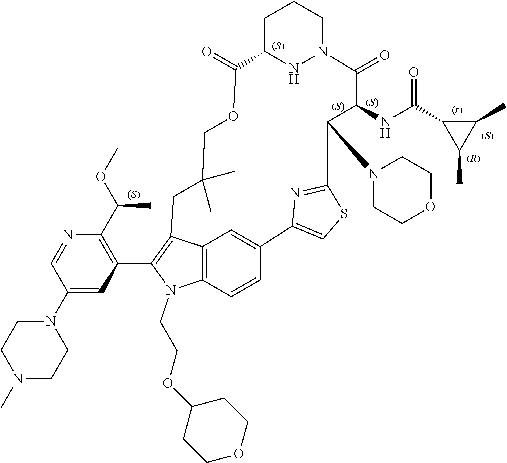 | B594 |
| 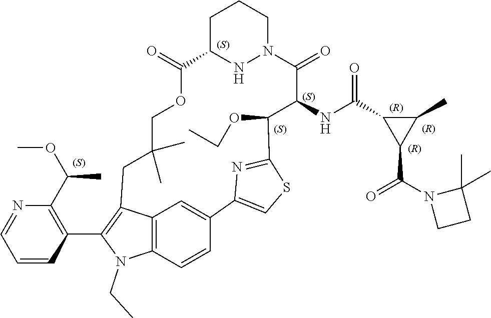 | B595 |
| 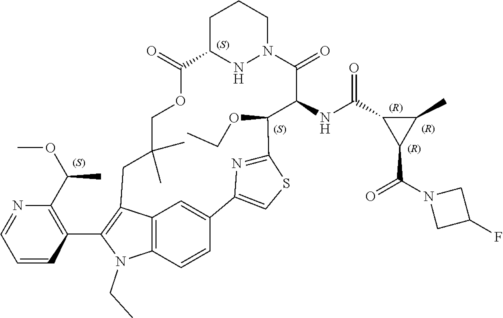 | B596 |
| 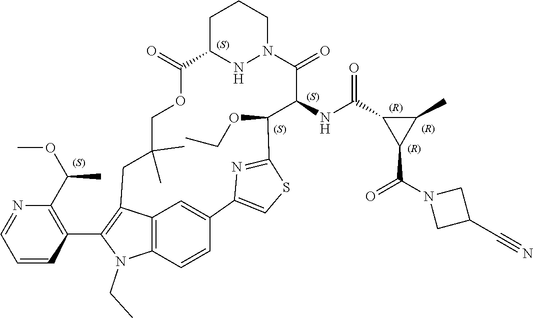 | B597 |
| 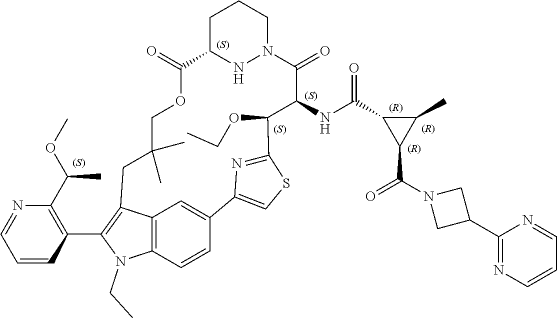 | B598 |
| 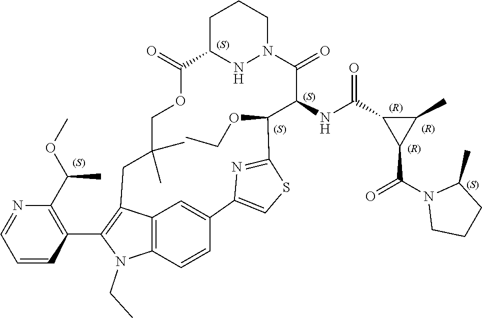 | B599 |
| 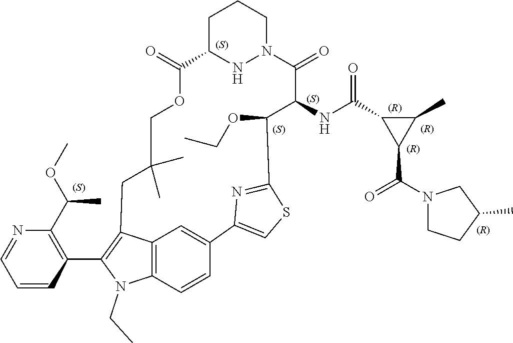 | B600 |
| 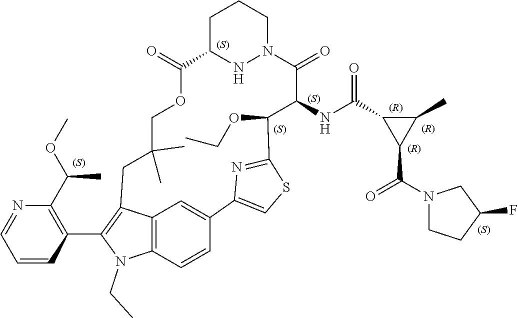 | B601 |
| 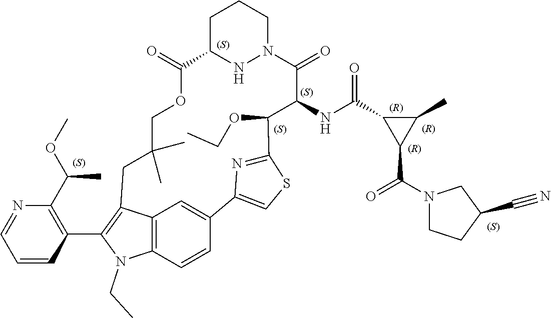 | B602 |
| 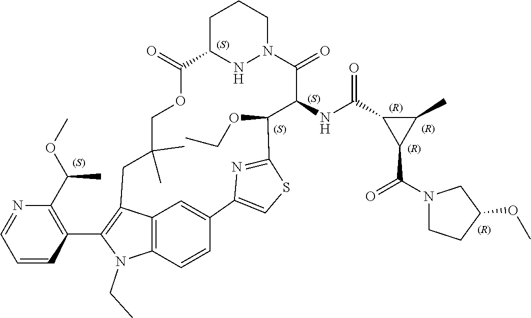 | B603 |
| 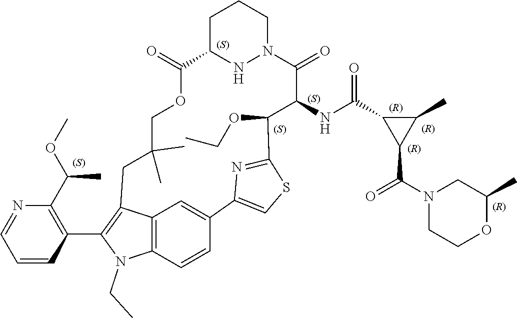 | B604 |
| 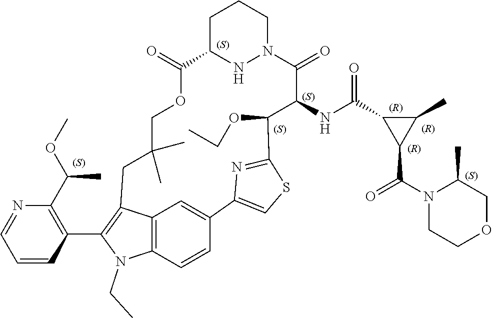 | B605 |
| 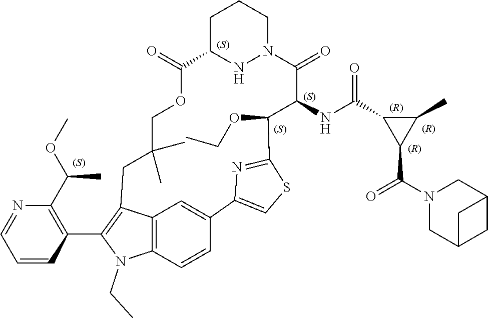 | B606 |
| 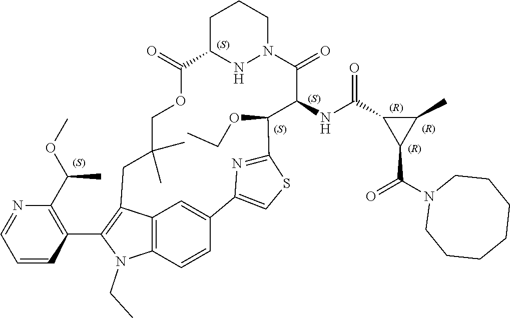 | B607 |
| 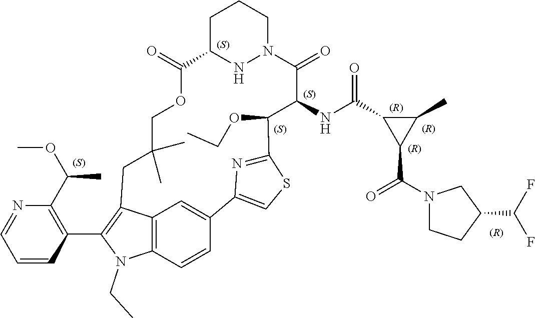 | B608 |

In some embodiments, a compound of the present invention has improved oral bioavailability (% F) compared to what is known in the art. Methods of measuring oral bioavailability are known in the art, and one such method is provided below:
Oral bioavailability may be determined in BALB/c mice. Following intravenous (IV) bolus and oral gavage (PO) administration of a test compound, about 30 μL of whole blood samples are collected at designated time points into tubes containing K₂EDTA. The blood samples are centrifuged at 4600 rpm at 4° C. for about 5 minutes and plasma samples are stored at −80° C. prior to bioanalysis. Plasma samples are extracted by protein precipitation and analyzed by tandem mass spectrometry (LC MS/MS) on, for example, an API 5500 system using electrospray positive ionization.

All PK parameters may be derived from plasma concentration over time data with non-compartment analysis using WinNonlin. The bioavailability (F %, also % F) is estimated using the following equation:

$$F \% = \frac{{AUC}_{inf , PO}}{{AUC}_{inf , IV}} \cdot \frac{{Dose}_{IV}}{{Dose}_{PO}}$$

AUC_{inf.PO} is the area under the plasma concentration over time from time zero to infinity following PO administration.
AUC_{inf.IV} is the area under the plasma concentration over time from time zero to infinity following IV administration.
Dose_{IV} is the total dose of IV administration
Dose_{PO} is the total dose of PO administration

In general, F % (or % F) values of over 30% are preferred, with values over 50% being more preferred.

In some embodiments, a compound of the present invention is selective for one or more particular Ras mutants over other Ras mutants or wild-type compared to what is known in the art. Methods of measuring such selectivity are known in the art, such as the Ras-Raf binding assay, a protocol for which is provided above. Accordingly, in some embodiments, compounds of the present invention are selective for KRAS^{G12V} over other Ras mutants or over wild-type, or combinations thereof. In some embodiments, compounds of the present invention exhibit an IC50 value of less than 1 uM for KRAS^{G12V} in the Ras-Raf binding assay described above. In some embodiments, compounds of the present invention exhibit at least 10× selectivity for KRAS^{G12V} versus wild-type KRAS as measured using the Ras-Raf binding assay.

In some embodiments, a compound of the present invention is more potent for one or more particular Ras mutants over other Ras mutants or wild-type compared to what is known in the art. Methods of measuring such potency are known in the art, such as the pERK assay, a protocol for which is provided in the Examples below. In some embodiments, compounds of the present invention exhibit greater potency with respect to KRAS^{G12V} than what is known in the art. Compounds of the present invention may also exhibit greater potency with respect to other RAS mutants disclosed herein, or combinations thereof. In some embodiments, a compound of the present invention exhibits at least 1.5× greater potency with respect to KRAS^{G12V} versus KRAS^{G12D} using the pERK assay.

In some embodiments, a compound of the present invention exhibits a greater detrimental effect on cell viability with respect to one or more particular Ras mutants over other Ras mutants or wild-type compared to what is known in the art. Methods of measuring cell viability are known in the art, such as the CellTiter-Glo® Cell Viability Assay, a protocol for which is provided in the Examples below. Accordingly, in some embodiments, compounds of the present invention exhibit a greater decrease in cell viability with respect to KRAS^{G12V} compared to what is known in the art. Compounds of the present invention may also exhibit a greater decrease in cell viability respect to other RAS mutants disclosed herein, or combinations thereof. In some embodiments, compounds of the present invention exhibit a greater decrease in cell viability with respect to KRAS^{G12V} versus wild-type KRAS.

In some embodiments, a compound of the present invention may exhibit greater metabolic stability, permeability, or solubility, or a combination thereof, versus what is known in the art. Methods for measuring such properties are known in the art. In some embodiments, a compound of the present invention may exhibit improvements with respect to any of the following properties, or a combination thereof, compared to what is known in the art: selectivity, potency, cell viability, metabolic stability, permeability, oral bioavailability, or solubility.

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

Compounds of the present invention are also adaptable for uses in antibody-drug conjugates as well as degrader applications.

Further provided is a method of treating cancer in a subject in need thereof, the method comprising administering to the subject a therapeutically effective amount of a compound of the present invention, or a pharmaceutically acceptable salt thereof. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer comprises a Ras mutation, such as K-Ras G12V. Other Ras mutations are described herein.

Further provided is a method of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof. For example, the Ras protein is K-Ras G12V. Other Ras proteins are described herein. The cell may be a cancer cell, such as a pancreatic cancer cell, a colorectal cancer cell, a lung cancer (e.g., non-small cell lung cancer cell), an acute myeloid leukemia cell, a multiple myeloma cell, a thyroid gland adenocarcinoma cell, a myelodysplastic syndrome cell, a melanoma cell, or a squamous cell lung carcinoma cell. Other cancer types are described herein. The cell may be in vivo or in vitro.

With respect to compounds of the present invention, one stereoisomer may exhibit better inhibition than another stereoisomer. For example, one atropisomer may exhibit inhibition, whereas the other atropisomer may exhibit little or no inhibition.

In some embodiments, a method or use described herein further comprises administering an additional anti-cancer therapy. In some embodiments, the additional anti-cancer therapy is a HER2 inhibitor, an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, or a combination thereof. In some embodiments, the additional anticancer therapy is a SHP2 inhibitor. In other embodiments, the additional anticancer agent or therapy is a pan-KRAS inhibitor (e.g., Pan KRAS-IN-1, BI-286, YL-17231). Other additional anti-cancer therapies are described herein.

In some embodiments, provided herein is a compound of Formula V:

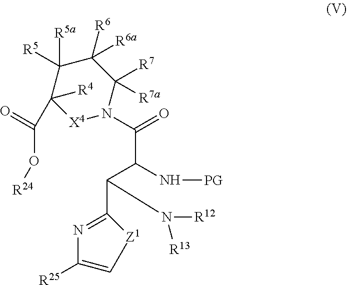

wherein:
R⁴, R⁵, R⁶, R⁷, R^{5a}, R^{6a} and R^{7a} are each independently hydrogen, halo or C₁-C₆ alkyl;
or R⁵ and R⁷ are taken together to form a methylene or ethylene bridge between the carbon atoms to which they are attached, and R⁴, R^{5a}, R⁶, Rea, and R^{7a} are each independently hydrogen, halogen, or C₁₋₆ alkyl;
or R⁴ and R⁶ are taken together to form a methylene or ethylene bridge between the carbon atoms to which they are attached, and R⁵, R^{5a}, R^{6a}, R⁷ and R^{7a} are each independently hydrogen, halogen, or C₁₋₆ alkyl;
or R⁵ and R⁶ taken together with the atoms to which they are attached form optionally substituted 3- to 7-membered cycloalkyl or optionally substituted 3- to 7-membered heterocycloalkyl;
or R⁶ and R⁷ taken together with the atoms to which they are attached form optionally substituted 3- to 7-membered cycloalkyl or optionally substituted 3- to 7-membered heterocycloalkyl;
or R⁵ and R^{5a} taken together with the atom to which they are attached form optionally substituted 3- to 7-membered cycloalkyl or an optionally substituted 3- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 3- to 7-membered heterocycloalkyl;
or R⁵ and R⁶ taken together form a bond;
X⁴ is NH or CH₂;
Z¹ is O or S;
PG is an amino protecting group;
R²⁴ is H or C₁₋₆ alkyl;
R²⁵ is halogen; and
R¹², and R¹³ are each, independently, optionally substituted C₁-C₆ alkyl, optionally substituted C₁-C₆ heteroalkyl, or R¹² and R¹³ taken together with the nitrogen atom to which they are attached form optionally substituted 3- to 11-membered heterocycloalkyl.

In some embodiments, X⁴ is NH.

In some embodiments, R²⁴ is C₁₋₆ alkyl.

In some embodiments, PG is Boc or Cbz.

In some embodiments, R²⁵ is Br or I.

In some embodiments, R¹² and R¹³ taken together with the nitrogen atom to which they are attached form optionally substituted 7- to 11-membered sprio heterocycloalkyl.

In some embodiments, R⁵ and R⁷ are taken together to form a methylene bridge between the carbon atoms to which they are attached, and R⁴, R^{5a}, R⁶, Roa, and R^{7a} are each independently hydrogen, halogen, or C₁₋₆ alkyl.

In some embodiments, the moiety:

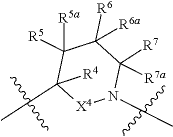

### in Formula (V) is

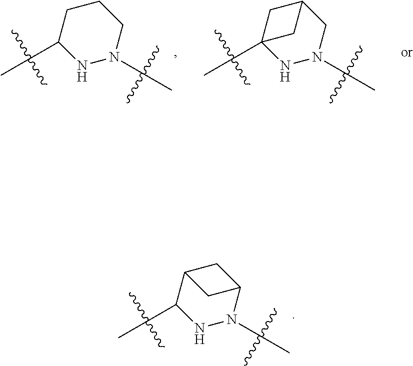 [SMILES: *C1CCCN(*)N1 ; *N1CC2CC(*)(C2)N1 ; *C1NN(*)C2CC1C2]

In some embodiments, the moiety:

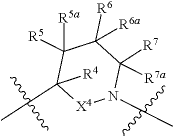

### in Formula (V) is

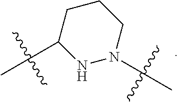 [SMILES: *C1CCCN(*)N1]

In some embodiments, the moiety:

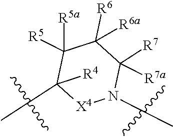

### in Formula (V) is

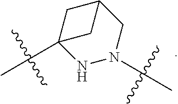 [SMILES: *N1CC2CC(*)(C2)N1]

In some embodiments, the moiety:

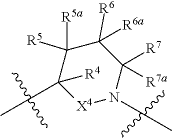

### in Formula (V) is

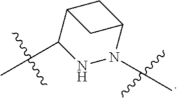 [SMILES: *C1NN(*)C2CC1C2]

In some embodiments, R⁵ and R⁷ are taken together to form a methylene or ethylene bridge between the carbon atoms to which they are attached, and R⁴, R⁵, R^{5a}, R⁶, R^{6a}, R⁷ and R^{7a} are each independently hydrogen, halogen, or C₁₋₆ alkyl.

In some embodiments, Z¹ is O. In some embodiments, Z¹ is S.

In some embodiments, provided herein is a compound of Formula (VI):

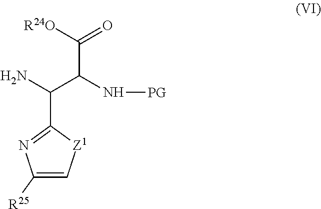

wherein:
Z¹ is O or S;
PG is an amino protecting group;
R²⁴ is H or C₁₋₆ alkyl; and
R²⁵ is halogen.

In some embodiments, Z¹ is O. In some embodiments, Z¹ is S.

In some embodiments, PG is Boc or Cbz.

In some embodiments, R²⁴ is C₁₋₆ alkyl.

In some embodiments, R²⁵ is Br or I.

In some embodiments, Z¹ is O, R²⁵ is I, PG is Boc and R¹⁴ is Et.

In some embodiments, provided herein is a compound of Formula VII:

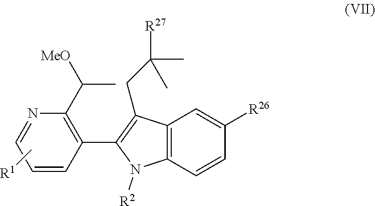

wherein:
R¹ is hydrogen, optionally substituted C₁-C₆ alkyl, optionally substituted C₂₋₆ alkenyl, optionally substituted C₂₋₆ alkynyl, optionally substituted C₁-C₆ heteroalkyl, optionally substituted 3- to 10-membered cycloalkyl, optionally substituted 3- to 6-membered cycloalkenyl, optionally substituted 3- to 15-membered heterocycloalkyl, optionally substituted 6 to 10-membered aryl, optionally substituted 5 to 10-membered heteroaryl, —B(OH)₂ or 4,4,5,5-tetramethyl-1,3,2-dioxaborolan-2-yl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6- to 10-membered aryl, optionally substituted 5- to 10-membered heteroaryl;
R²⁶ is halogen, toluenesulfonyloxy, CH₃SO₃—, CF₃SO₃—or —B(OH)₂; and
R²⁷ is —CH₂—OH, —CH₂O-PG¹, —COOH or —COOR²⁸;
R²⁸ is C₁₋₆ alkyl or NR²⁹R²⁹;
R²⁹ is H or independently C₁₋₆ alkyl; and
PG¹ is a hydroxy protecting group.

In some embodiments, R¹ is H, 4-methylpiperazin-1-yl, 4-cyclopropylpiperazin-1-yl, —B(OH)₂ or 4,4,5,5-tetramethyl-1,3,2-dioxaborolan-2-yl.

In some embodiments, R¹ is —B(OH)₂ or 4,4,5,5-tetramethyl-1,3,2-dioxaborolan-2-yl and R²⁶ is Br or I.

In some embodiments, R² is H, C₁₋₆ alkyl, 2-((tetrahydro-2H-pyran-4-yl)oxy)ethyl, or 3-cyanocyclobutyl.

In some embodiments, R²⁶ is Br or I.

In some embodiments, PG¹ is TBDPS.

In some embodiments, R²⁷ is —CH₂OH.

In some embodiments, the compound of Formula (VII) is selected from:

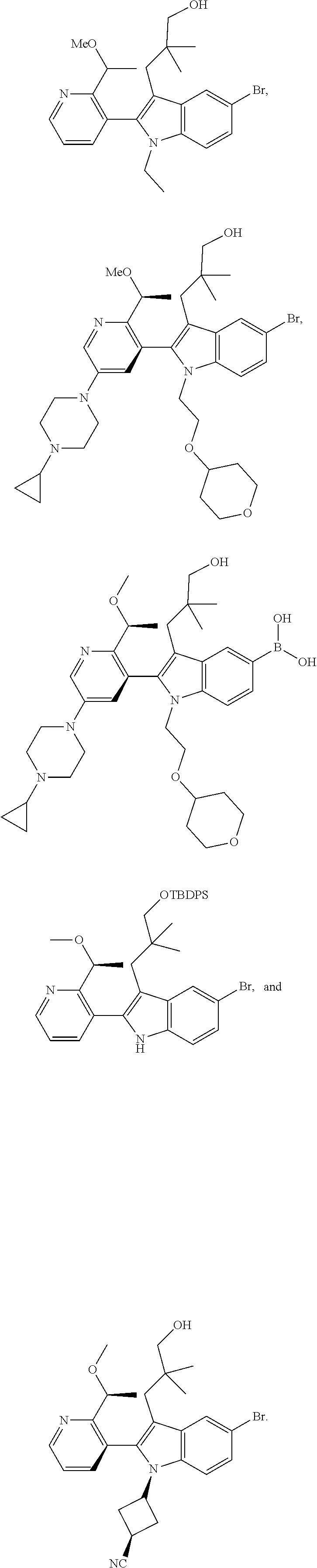

In some embodiments, provided herein is a compound of Formula (VIII):

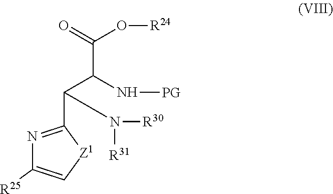

wherein:
Z¹ is O or S;
PG is H or an amino protecting group;
R³⁰ and R³¹ are each H; or R³⁰ and R³¹ taken together with the nitrogen atom to which they are attached form optionally substituted 7- to 11-membered spiro heterocycloalkyl;
R²⁴ is H or C₁₋₆ alkyl; and
R²⁵ is Br or I.

In some embodiments, R²⁴ is H.

In some embodiments, PG is Boc.

In some embodiments, R³⁰ and R³¹ are each H.

In some embodiments, R³⁰ and R³¹ taken together with the nitrogen atom to which they are attached form an optionally substituted 7- to 11-membered spiro heterocycloalkyl.

In some embodiments, Z¹ is O. In some embodiments, Z¹ is S.

In some embodiments, the compound of Formula (VIII) is:

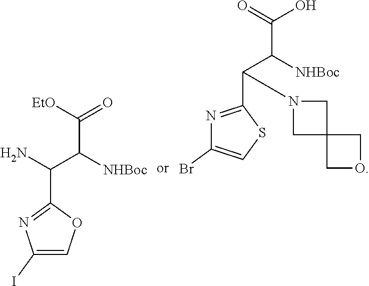 [SMILES: CCOC(=O)C(NC(=O)OC(C)(C)C)C(N)c1nc(I)co1 ; CC(C)(C)OC(=O)NC(C(=O)O)C(c1nc(Br)cs1)N1CC2(COC2)C1]

In some embodiments, provided herein is a compound of Formula (IX):

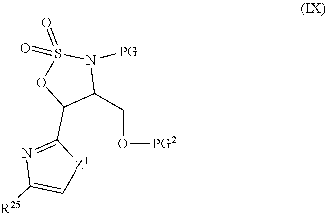

wherein:
Z¹ is O or S;
PG is an amino protecting group;
PG² is a hydroxy protecting group; and
R²⁵ is Br or I.

In some embodiments, PG is Boc.

In some embodiments, PG² is —C(O) CH₃.

In some embodiments, Z¹ is O. In some embodiments, Z¹ is S.

In some embodiments, R²⁵ is Br.

In some embodiments, provided herein is a compound of Formula (X):

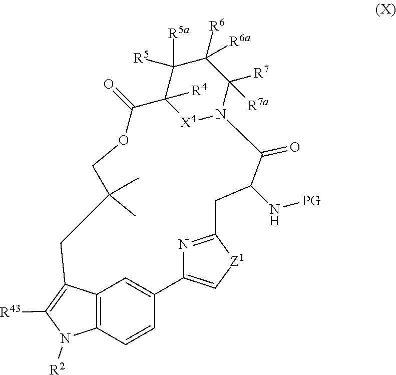

wherein PG is H or an amino protecting group;
Z¹ is O or S;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6- to 10-membered aryl, optionally substituted 5- to 10-membered heteroaryl;
R³² is halogen, —B(OH)₂ or 4,4,5,5-tetramethyl-1,3,2-dioxaborolan-2-yl;
R⁴, R⁵, R⁶, R⁷, R^{5a}, R^{6a} and R^{7a} are each independently hydrogen, halo or C₁-C₆ alkyl;
or R⁵ and R⁷ are taken together to form a methylene or ethylene bridge between the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form a methylene or ethylene bridge between the carbon atoms to which they are attached, and R⁵, R^{5a}, R^{6a}, R⁷ and R^{7a} are each independently hydrogen, halogen, or C₁₋₆ alkyl;
or R⁵ and R⁶ taken together with the atoms to which they are attached form optionally substituted 3- to 7-membered cycloalkyl or optionally substituted 4- to 7-membered heterocycloalkyl;
or R⁶ and R⁷ taken together with the atoms to which they are attached form optionally substituted 3- to 7-membered cycloalkyl or optionally substituted 4- to 7-membered heterocycloalkyl;
or R⁵ and R^{5a} taken together with the atom to which they are attached form optionally substituted 3- to 7-membered cycloalkyl or an optionally substituted 4- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 4- to 7-membered heterocycloalkyl;
or R⁵ and R⁶ taken together form a bond.

In some embodiments, PG is Boc.

In some embodiments, R² is H or optionally substituted C₁₋₆ alkyl.

In some embodiments, R³² is 4,4,5,5-tetramethyl-1,3,2-dioxaborolan-2-yl, Br or I.

In some embodiments, Z¹ is O. In some embodiments, Z¹ is S.

In some embodiments, R⁴, R⁵, R⁶, R⁷, R^{5a}, R^{6a} and R^{7a} are each hydrogen.

In some embodiments, R⁵ and R⁷ are taken together to form a methylene bridge between the carbon atoms to which they are attached, and R⁴, R^{5a}, R⁶, R^{6a}, and R^{7a} are each hydrogen.

In some embodiments, provided herein is a compound of Formula (XI):

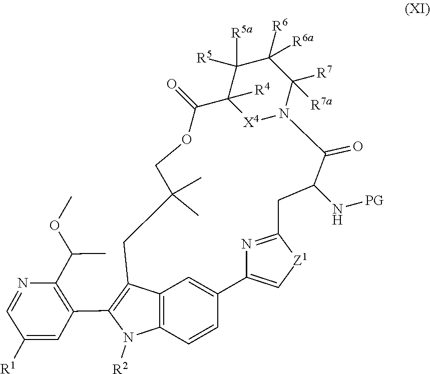

wherein:
PG is H or an amino protecting group;
Z¹ is O or S;
R¹ is hydrogen, optionally substituted C₁-C₆ alkyl, optionally substituted C₂₋₆ alkenyl, optionally substituted C₂₋₆ alkynyl, optionally substituted C₁-C₆ heteroalkyl, optionally substituted 3- to 10-membered cycloalkyl, optionally substituted 3- to 6-membered cycloalkenyl, optionally substituted 3- to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6- to 10-membered aryl, optionally substituted 5- to 10-membered heteroaryl;
R⁴, R⁵, R⁶, R⁷, R^{5a}, R^{6a} and R^{7a} are each independently hydrogen, halo or C₁-C₆ alkyl; or R⁵ and R⁷ are taken together to form a methylene or ethylene bridge between the carbon atoms to which they are attached, and R⁴, R^{5a}, R⁶, Roa, and R^{7a} are each independently hydrogen, halogen, or C₁₋₆ alkyl;
or R⁴ and R⁶ are taken together to form a methylene or ethylene bridge between the carbon atoms to which they are attached, and R⁵, R^{5a}, R^{6a}, R⁷ and R^{7a} are each independently hydrogen, halogen, or C₁₋₆ alkyl;
or R⁵ and R⁶ taken together with the atoms to which they are attached form optionally substituted 3- to 7-membered cycloalkyl or optionally substituted 4- to 7-membered heterocycloalkyl;
or R⁶ and R⁷ taken together with the atoms to which they are attached form optionally substituted 3- to 7-membered cycloalkyl or optionally substituted 4- to 7-membered heterocycloalkyl;
or R⁵ and R^{5a} taken together with the atom to which they are attached form optionally substituted 3- to 7-membered cycloalkyl or an optionally substituted 4- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 4- to 7-membered heterocycloalkyl;
or R⁵ and R⁶ taken together form a bond.

In some embodiments, PG is H or Boc.

In some embodiments, R¹ is H or optionally substituted heterocycloalkyl.

In some embodiments, R² is H or optionally substituted C₁₋₆ alkyl.

In some embodiments, Z¹ is O. In some embodiments, Z¹ is S.

In some embodiments, R⁴, R⁵, R⁶, R⁷, R^{5a}, R^{6a} and R^{7a} are each hydrogen.

In some embodiments, R⁵ and R⁷ are taken together to form a methylene bridge between the carbon atoms to which they are attached, and R⁴, R^{5a}, R⁶, R^{6a}, and R^{7a} are each hydrogen.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Scheme below, together with synthetic methods known in the art of synthetic organic chemistry, or variations thereon as appreciated by those skilled in the art. As a further example, synthetic methods described in WO 2020/132597, WO 2021/091982, WO 2021/091967, WO 2021/091956, WO 2022/060836 (e.g., Examples A225 and A387), WO 2022/235864, WO 2022/235870, WO 2023/060253, and WO 2023/133543, the disclosure of each of which is incorporated herein by reference, may be useful in preparing compounds of the invention. These methods include but are not limited to those methods described in the Scheme below.

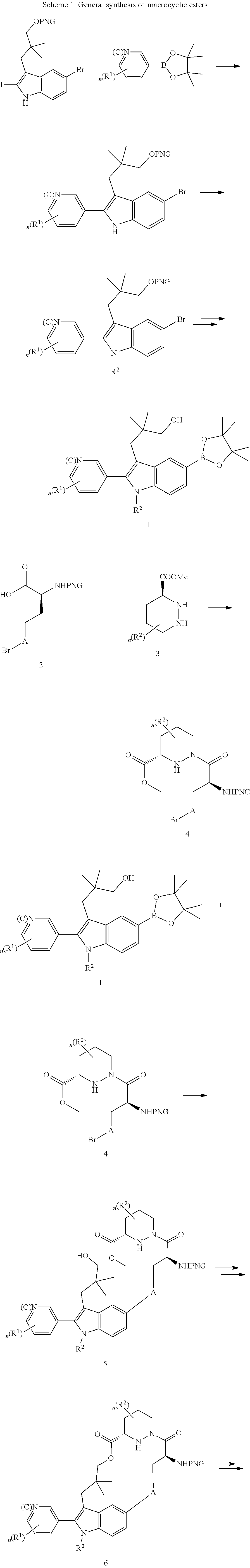

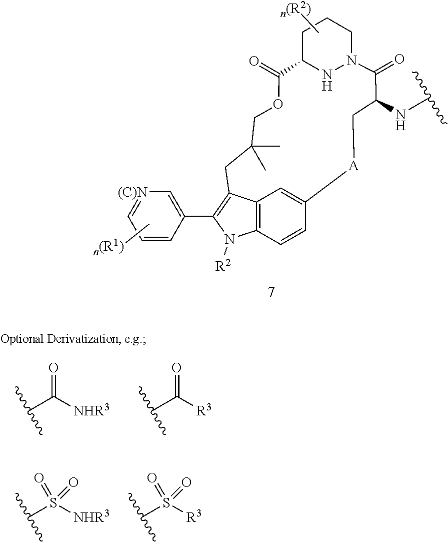

A general synthesis of macrocyclic esters is outlined in Scheme 1. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl(S)-hexahydropyridazine-3-carboxylate analogs (3) can be prepared by a variety of methods highlighted below.

Methyl-amino-3-(4-bromothiazol-2-yl) propanoyl) hexahydropyridazine-3-carboxylate (4) can be prepared via coupling of(S)-2-amino-3-(4-bromothiazol-2-yl) propanoic acid (2) with methyl(S)-hexahydropyridazine-3-carboxylate derivative (3).

The final macrocyclic esters can be made by coupling of methyl-amino-3-(4-bromothiazol-2-yl) propanoyl) hexahydropyridazine-3-carboxylate (4) and an appropriately substituted indolyl boronic ester (1) in the presence of Pd catalyst followed by hydrolysis and macrolactonization steps to result in an appropriately protected macrocyclic intermediate (5). Deprotection and coupling with an appropriately substituted carboxylic acid (or other coupling partner) results in a macrocyclic product. Additional deprotection or functionalization steps could be required to produce a final compound (6).

Further, with respect to Scheme 1, the thiazole may be replaced with an alternative optionally substituted 5 to 6-membered heteroarylene, an optionally substituted 3 to 6-membered cycloalkylene, optionally substituted 3 to 6-membered heterocycloalkylene (e.g., morpholino), or optionally substituted 6-membered arylene (e.g., phenyl). PNG is a hydroxy protecting group when attached to the oxygen atom of the molecule and as an amino protecting group when attached to the nitrogen atom (e.g., in compound 4). The substituents R¹ and R² and moiety A in Scheme 1 are as defined in the specification and claims. The subscript n is 1, 2 or 3. R³ corresponds to variable W, as defined in the specification and claims.

In some embodiments, a general synthesis of macrocyclic esters is outlined in Scheme 1A.

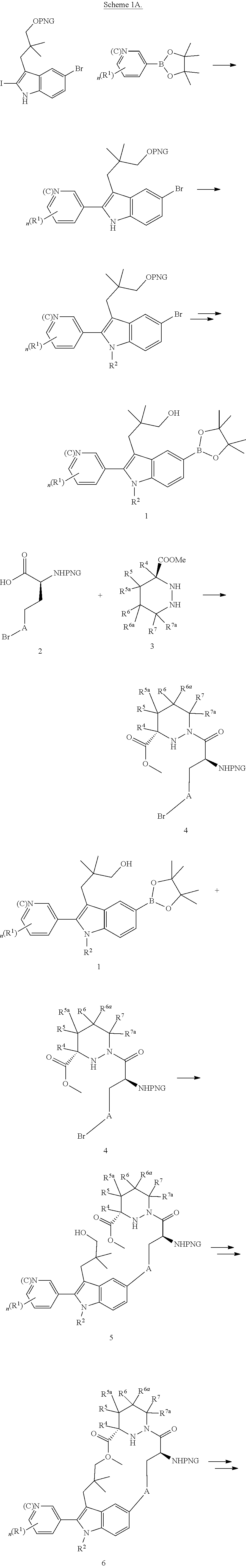

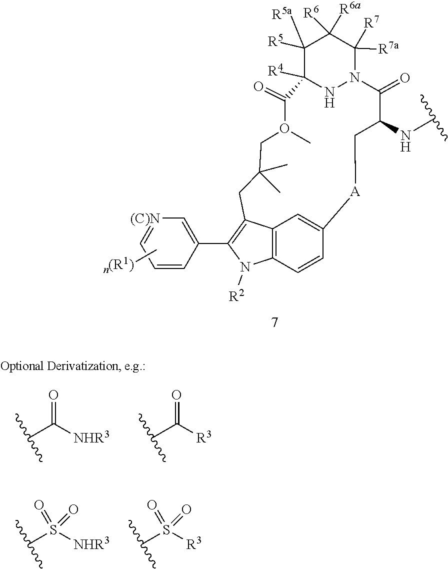

As shown in Schemes 1 and 1A, PNG is a hydroxy protecting group when attached to the oxygen atom of the molecule. PNG is an amino protecting group when attached to the nitrogen atom (e.g., in compound 4). The substituents R¹, R², R⁴, R^{4a}, R⁵, R^{5a}, R⁶, R^{6a}, R⁷, R^{7a} and moiety A are defined in the specification and claims. R³ corresponds to variable W, as defined in the specification and claims.

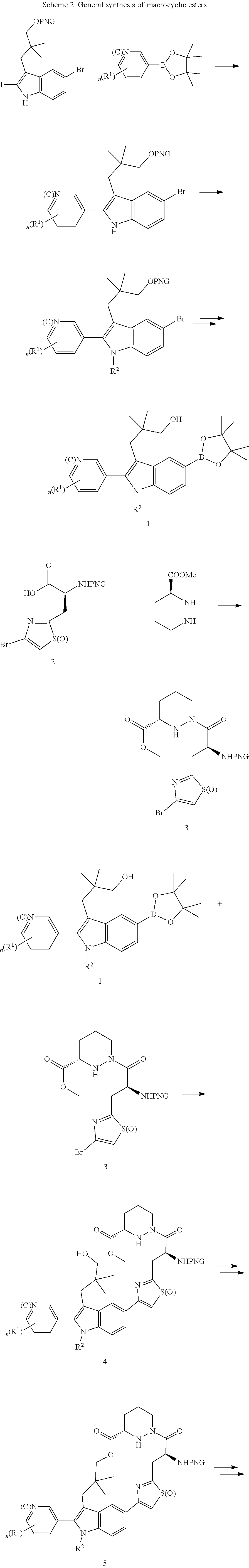

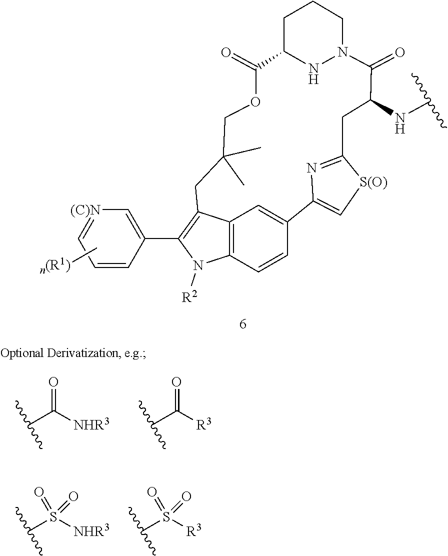

A further general synthesis of macrocyclic esters is outlined in Scheme 2. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl) propanoyl) hexahydropyridazine-3-carboxylate (3) can be prepared via coupling of(S)-2-amino-3-(4-bromothiazol-2-yl) propanoic acid (2) with methyl(S)-hexahydropyridazine-3-carboxylate (3).

The final macrocyclic esters can be made by coupling of methyl-amino-3-(4-bromothiazol-2-yl) propanoyl) hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (4). Deprotection and coupling with an appropriately substituted carboxylic acid (or other coupling partner) results in a macrocyclic product. Additional deprotection or functionalization steps could be required to produce a final compound (6).

Further, with respect to Scheme 1, the thiazole may be replaced with an alternative optionally substituted 5 to 6-membered heteroarylene, an optionally substituted 3 to 6-membered cycloalkylene, optionally substituted 3 to 6-membered heterocycloalkylene (e.g., morpholino), or optionally substituted 6-membered arylene (e.g., phenyl). PNG is a hydroxy protecting group when attached to the oxygen atom of the molecule and as an amino protecting group when attached to the nitrogen atom of the molecule (e.g., in compound 3). The substituents R¹ and R² in Scheme 2 are as defined in the specification and claims. The subscript n is 1, 2 or 3. R³ corresponds to variable W, as defined in the specification and claims.

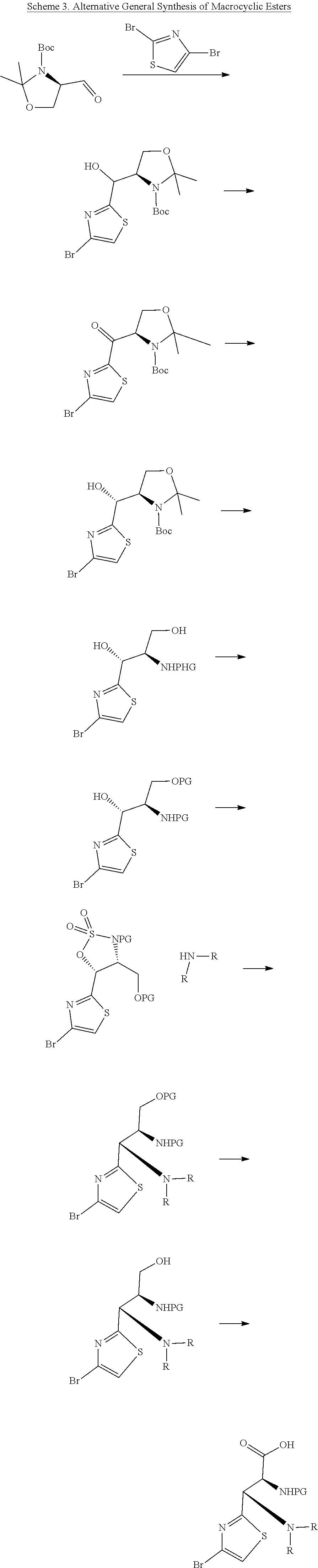

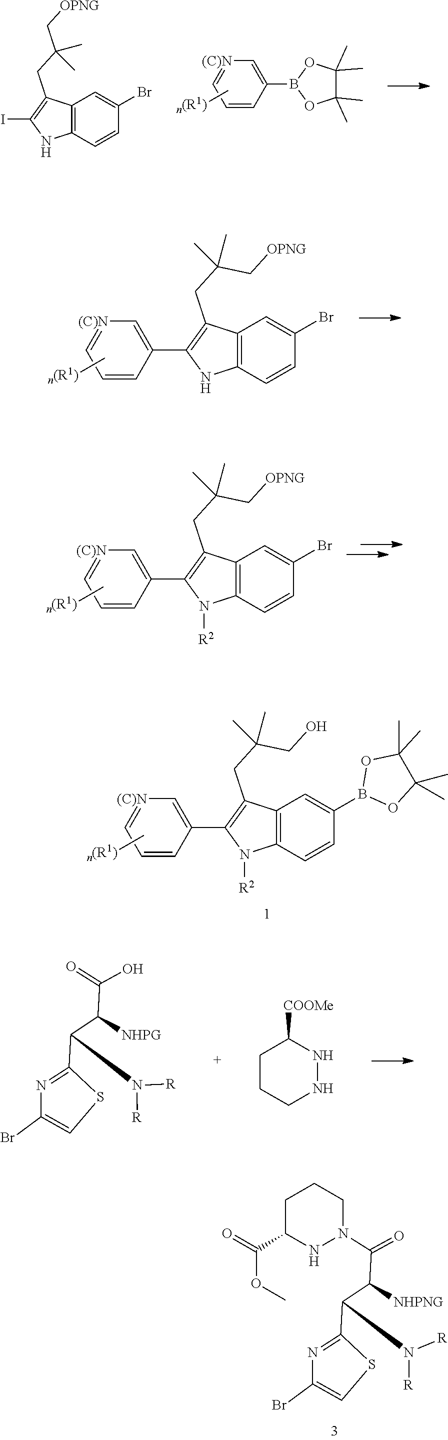

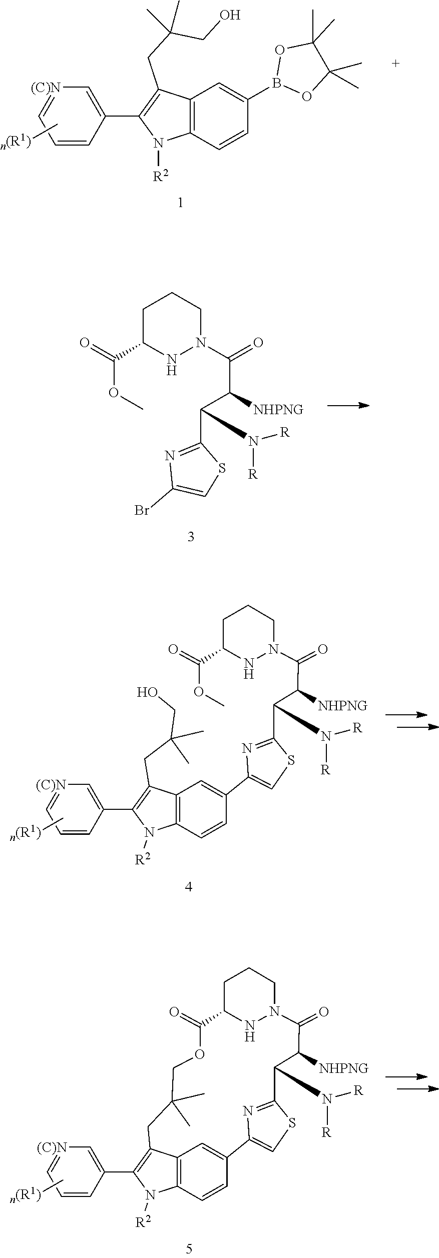

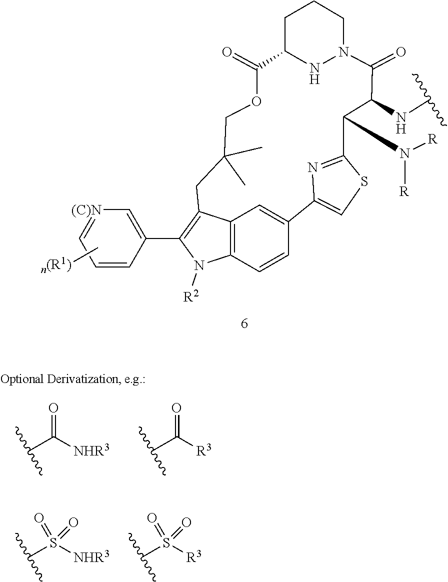

A further general synthesis of macrocyclic esters is outlined in Scheme 3. An appropriately substituted indolyl boronic ester (1) can be prepared as described in Schemes 1 and 2.

Amino-substituted(S)-2-amino-3-(4-bromothiazol-2-yl) propanoic acid can be prepared from 2,4-dibromothiazole as shown in Scheme 3, above.

Amino-substituted methyl-amino-3-(4-bromothiazol-2-yl) propanoyl) hexahydropyridazine-3-carboxylate (3) can be prepared via coupling of amino-substituted(S)-2-amino-3-(4-bromothiazol-2-yl) propanoic acid with methyl(S)-hexahydropyridazine-3-carboxylate.

The final macrocyclic esters can be prepared in the manner described in Schemes 1 and 2. As shown in Scheme 3, PG is a hydroxy protecting group when attached to the oxygen atom of the molecule and as an amino protecting group when attached to the nitrogen atom of the molecule. PNG is a hydroxy protecting group when attached to oxygen and an amino protecting group when attached to nitrogen. The substituents R¹ and R² are as defined in the specification and claims. The subscript n is 1, 2 or 3. R³ corresponds to variable W, as defined in the specification and claims. The two R substituents on the nitrogen atom in compounds 3, 4, 5 and 6 correspond to substituents R¹² and R¹³, also defined in the specification and claims.

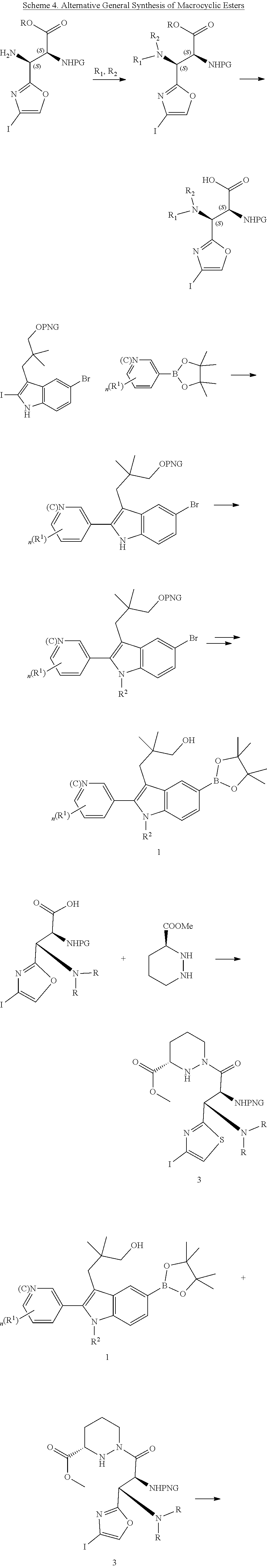

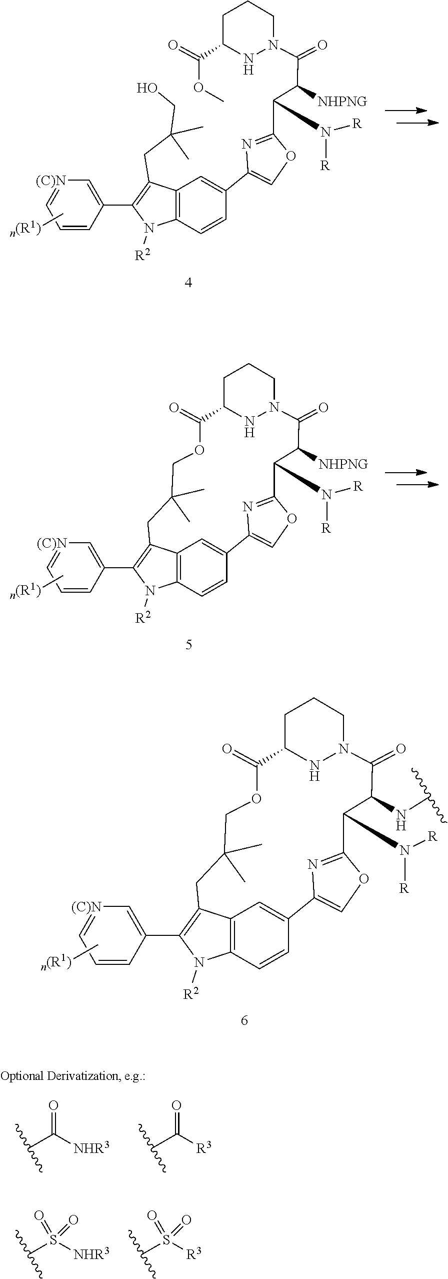

Oxazole-containing compounds can be prepared in an analogous method as shown in Schemes 1-3 but with diamino-substituted iodo-oxazole (3). This compound can be prepared as shown in Scheme 4, above.

The final macrocyclic esters can be prepared in the manner described in Schemes 1 and 2. Compounds with various substitutions on the pyridazine ring, as disclosed and claimed herein, can be prepared using methods known in the art and described in WO2024067857, which is incorporated by reference in its entirety for all purposes.

As shown in Scheme 4, PG represents an amino protecting group when attached to the nitrogen atom of the molecule. PNG is a hydroxy protecting group when attached to the oxygen atom of the molecule and as an amino protecting group when connected to nitrogen. The substituents R¹ and R² are as defined in the specification and claims. The subscript n is 1, 2 or 3. R³ corresponds to variable W, as defined in the specification and claims. The two R substituents on the nitrogen atom in compounds 3, 4, 5 and 6 correspond to substituents R¹² and R¹³, also defined in the specification and claims. The R¹ and R² substituents correspond to substituents R¹² and R¹³, as defined in the specification and claims. In some embodiments, the R group in

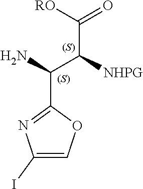

is hydrogen or an optionally substituted alkyl.

The synthetic routes described in the general schemes are suitable for synthesizing compounds in which the hexahydropyridazine moiety:

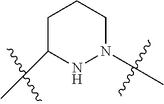 [SMILES: *C1CCCN(*)N1]

is replaced by:

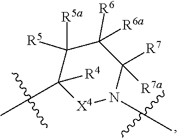

as detailed in the specification and claims.

### Pharmaceutical Compositions and Methods of Use

The compounds with which the invention is concerned are Ras inhibitors and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder, or condition or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating, or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive, or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, intradermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous, or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

In some embodiments, a compound described herein, or a compound of any of formulas I′ or I, or any of the subformulas described herein, or a compound recited in any of the claims, or a pharmaceutically acceptable salt, solvate, tautomer or isomer thereof, can promote, enhance, or increase GTP hydrolysis in a RAS mutant protein. Methods of measuring such hydrolysis are known in the art: see, e.g., WO 2024206858, incorporated herein by reference in its entirety.

### Methods of Use

In some embodiments, the invention discloses a method of treating a disease or disorder that is characterized by aberrant Ras activity due to a Ras mutant. In some embodiments, the disease or disorder is a cancer.

Accordingly, also provided is a method of treating cancer in a subject in need thereof, the method comprising administering to the subject a therapeutically effective amount of a compound of the present invention, or a pharmaceutically acceptable salt thereof, or a pharmaceutical composition comprising such a compound or salt. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a Ras protein-related disorder in a subject in need thereof, the method comprising administering to the subject a therapeutically effective amount of a compound of the present invention, or a pharmaceutically acceptable salt thereof, or a pharmaceutical composition comprising such a compound or salt.

In some embodiments, the compounds of the present invention or pharmaceutically acceptable salts thereof, pharmaceutical compositions comprising such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate, and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma, and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms), multiple myeloma, myelodysplastic syndrome), Hodgkin's disease, non-Hodgkin's lymphoma (malignant lymphoma); Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the Ras protein is wild type (Ras^{WT}). Accordingly, in some embodiments, a compound of the present invention is employed in a method of treating a patient having a cancer comprising a Ras^{WT} (e.g., K-Ras^{WT}, H-Ras^{WT} or N-Ras^{WT}). In some embodiments, the Ras protein is Ras amplification (e.g., K-Ras^{amp}). Accordingly, in some embodiments, a compound of the present invention is employed in a method of treating a patient having a cancer comprising a Ras^{amp} (K-Ras^{amp}, H-Ras^{amp} or N-Ras^{amp}). In some embodiments, the cancer comprises a Ras mutation, such as a Ras mutation described herein. In some embodiments, a mutation is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof;
   or a combination of any of the foregoing. In some embodiments, the cancer comprises a K-RasG12V mutation and the cancer is colorectal cancer. In some embodiments, the cancer comprises a K-RasG12V mutation and the cancer is pancreatic cancer. In some embodiments, the cancer comprises a K-RasG12V mutation and the cancer is non-small cell lung cancer.

Methods of detecting Ras mutations are known in the art. Such means include, but are not limited to direct sequencing, and utilization of a high-sensitivity diagnostic assay (with CE-IVD mark), e.g., as described in Domagala, et al., Pol J Pathol 3:145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

Also provided is a method of inhibiting a Ras protein in a cell, the method comprising contacting the cell with an effective amount of a compound of the present invention, or a pharmaceutically acceptable salt thereof. A method of inhibiting RAF-Ras binding, the method comprising contacting the cell with an effective amount of a compound of the present invention, or a pharmaceutically acceptable salt thereof, is also provided. The cell may be a cancer cell. The cancer cell may be of any type of cancer described herein. The cell may be in vivo or in vitro.

### Combination Therapy

The methods of the invention may include a compound of the present invention used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65: S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In certain embodiments, compositions of the disclosure comprise a compound of the present invention and one additional therapeutic agent. In certain embodiments, compositions of the disclosure comprise a compound of the present invention and two additional therapeutic agents. In certain embodiments, compositions of the disclosure comprise a compound of the present invention and three additional therapeutic agents. In certain embodiments, compositions of the disclosure comprise a compound of the present invention and four or more additional therapeutic agents.

Also provided are pharmaceutical compositions including the combinations, or a pharmaceutically acceptable salt thereof, and a pharmaceutically acceptable excipient. Compositions comprising a combination of therapeutic agents may be used in methods of modulating RAS (e.g., in a subject or in a cell) and in methods of treating RAS related diseases and disorders (e.g., cancer), as described herein. The present disclosure provides, inter alia, compositions, methods, and kits for treating or preventing a RAS related disease or disorder.

Exemplary agents that may be used in combination with a compound of the present invention are described below. All references herein are incorporated by reference for the agents described, including compound or molecular structures disclosed therein, whether explicitly stated as such or not.

### a) RAS (ON) Inhibitors

Compositions and methods of the present disclosure may include a compound of the present invention plus a RAS (ON) inhibitor. In some embodiments, the RAS (ON) inhibitor is RMC-6236, RMC-7977, RMC-6291, RMC-4998, RMC-9805, RMC-044, RMC-5127, GFH547, ERAS-0015 or compound 6A of WO 2024/067857. Exemplary RAS (ON) inhibitors useful in combinations according to the present disclosure can be found in any one of the following patent applications: WO 2024206858, WO 2024169914, WO 202453208, WO 2024206858, WO 2024149819, WO 2024104364, WO 2024067857, WO 2024060966, WO 2024017859, WO 2024102421, WO 2024008834, WO 2024008610, WO 2023232776, WO 2023240263, WO 2023086341, WO 2023208005, WO 2023133543, WO 2023060253, WO 2023025832, WO 2023015559, WO 2023060253, WO 2022235870, WO 2022235864, WO 2022060836, WO 2021091982, WO 2021091967, WO 2021091956, WO 2020132597, CN 117903169, CN 117720556, CN 117720555, CN 117720554, CN 117534687, CN 117534685, CN 117534684PCT/US2024/030993, PCT/US2024/023272, and PCT/US2024/023208, each of which is incorporated herein by reference in its entirety, including the compound structures disclosed therein.

In some embodiments, the RAS (ON) inhibitor is the multi-selective inhibitor RMC-6236

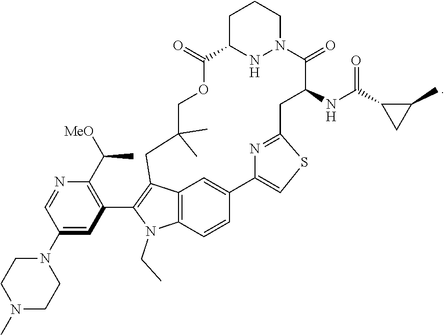

In some embodiments, a RAS (ON) multi-selective inhibitor is compound 6A of WO 2024/067857

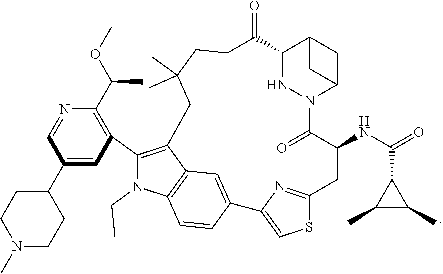

Some embodiments of combinations comprising a compound of the present invention include a composition comprising a RAS (ON) mutant-selective inhibitor. In some embodiments, the RAS (ON) mutant-selective inhibitor is a RAS (ON) G12C-selective inhibitor. In some embodiments, the RAS (ON) mutant-selective inhibitor is a RAS (ON) G12D-selective inhibitor. In some embodiments, the RAS (ON) mutant-selective inhibitor is a RAS (ON) G13C-selective inhibitor. In some embodiments, the RAS (ON) mutant-selective inhibitor is a RAS (ON) Q61H-selective inhibitor. In some embodiments, the RAS (ON) mutant-selective inhibitor is a RAS (ON) G13D-selective inhibitor.

In some embodiments, the RAS (ON) mutant-selective inhibitor useful according to the present disclosure is RMC-9805, a G12D mutant-selective inhibitor

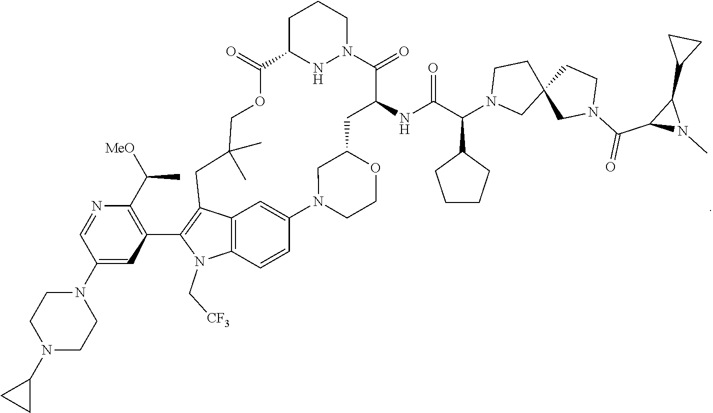

In some embodiments, the RAS (ON) mutant-selective inhibitor is the RAS (ON) G12C-selective tri-complex inhibitor, RMC-6291

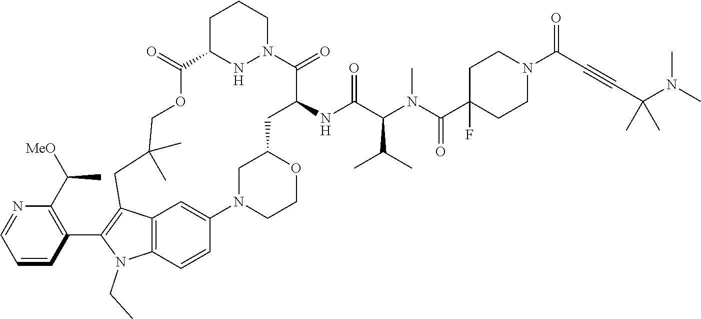

### (Schulze et. al., Science. 2023 Aug. 18; 381 (6659): 794-799).

In some embodiments, the combination therapy comprising a compound of the present invention may include one or more RAS (ON) inhibitors, for example, a compound of the present invention plus one or more RAS (ON) multi-selective inhibitors and/or one or more RAS (ON) mutant-selective inhibitors.

### b) RAS/MAPK Inhibitors

Compositions and methods described herein may include a compound of the present invention in combination with one or more RAS/MAPK pathway inhibitors. The RAS/MAPK pathway is a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions. In some embodiments, a therapeutic agent that may be combined with a RAS (ON) inhibitor is an inhibitor of the MAP kinase (MAPK) pathway (or “MAPK pathway inhibitor”). MAPK pathway inhibitors include, but are not limited to, one or more MAPK pathway inhibitors described in Cancers (Basel) 2015 September; 7 (3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH₅₁₂₆₇₆₆; MAP855; AZD6244; refametinib (RDEA 119/BAY 86-9766); GDC-0973/XL581; AZD8330 (ARRY-424704/ARRY-704); RO5126766 (Roche, described in PLOS One. 2014 Nov. 25; 9 (11)); and GSK1120212 (or JTP-74057, described in Clin Cancer Res. 2011 Mar. 1; 17 (5): 989-1000). The MAPK pathway inhibitor may be PLX8394, LXH254, GDC-5573, or LY3009120. A MAPK pathway inhibitor may be a PI3Kα: RAS breaker, such as BBO-10203.

i)RAS (OFF) Inhibitors and RAS (OFF) Degraders

Compositions and methods described herein may include a compound of the present invention in combination with one or more RAS (OFF) inhibitors. Numerous mutant-selective and pan-KRAS inhibitors have been disclosed and are known in the art. A RAS (OFF) inhibitor may be administered or formulated in combination with a RAS (ON) inhibitor described herein. RAS (OFF) inhibitors are designed to inhibit RAS activity by targeting different regions of the RAS protein in its inactive state (GDP bound state), preventing its activation and downstream signaling.

In some embodiments, a RAS (OFF) inhibitor is a KRAS (OFF) inhibitor that has a molecular weight of under 700 Da. The term “KRAS (OFF) inhibitor” refers to any RAS (OFF) inhibitor that binds to KRAS in its GDP-bound “OFF” position. In some embodiments, the KRAS (OFF) inhibitor is specific for a KRAS^{G12C} mutation. KRAS^{G12C} (OFF) inhibitors use a covalent binding group that allows them to selectively target the KRASG12C mutant protein, and many such inhibitors comprise a pyrimidine core. KRAS^{G12C} (OFF) inhibitors all target the same cysteine residue in the KRAS^{G12C} mutant protein, leading to a conformational change that locks the protein in an inactive state. KRAS^{G12C} (OFF) inhibitors include, but are not limited to, adagrasib (MRTX849), divarasib (RG6330/GDC-6036), fulzerasib (IBI351/GFH925), garsorasib (D-1553), glecirasib (JAB-21822), olomorasib (LY3537982), opnurasib (JDQ443), sotorasib (AMG 510), ARS-853, ARS-1620, BI 1823911, BPI-421286, D3S-001, GEC255, HBI-2438, HS-10370, JAB-21000, JAB-21822, JMKX001899, JNJ-74699157 (ARS-3248), MK-1084 and YL-15293. In some embodiments, the KRAS (OFF) inhibitor is selected from AMG 510 and MRTX849. In some embodiments, the KRAS (OFF) inhibitor is sotorasib (AMG 510). In some embodiments, the KRAS (OFF) inhibitor is adagrasib (MRTX849). In some embodiments, the KRAS (OFF) inhibitor is divarasib (GDC-6036). In some embodiments, the KRAS (OFF) inhibitor is selected from BPI-421286, JNJ-74699157 (ARS-3248), LY3537982, MRTX1257, ARS853, ARS1620, or GDC-6036.

In some embodiments, a KRAS (OFF) inhibitor is specific for a KRAS^{G12D} mutation. Many KRAS^{G12D} (OFF) inhibitors have been developed using KRAS^{G12C} (OFF) inhibitors as a starting point, thus sharing the backbone of G12C inhibitors in combination with other chemical moieties such as piperazine-based compounds. Non-limiting examples of KRAS^{G12D} (OFF) inhibitors include ASP3082, BPI-501836, ERAS-4693, ERAS-5024, HBW-012-D, HBW-012-E, HRS-4642, JAB-22000, KD-8, TSN1611, LY3962673, MRTX282, MRTX1133, Q2a, SHR1127, TH-Z827, TH-Z835, TSN1611, VRTX153 and DN022150.

In some embodiments, the small molecule RAS (OFF) inhibitor is specific for a KRAS^{G12V} mutation, such as JAB-23000 or QTX3544. In some embodiments, the small molecule RAS (OFF) inhibitor is specific for a KRAS^{G13D} mutation. In some embodiments, the small molecule RAS (OFF) inhibitor is a pan-KRAS (OFF) inhibitor, such as A2A-03, ABREV01, ABT-200, ADT-030, ADT-1004, AN9025, BBP-454, BGB-53038, BI-2865, BI 3706674, ERAS-4, ERAS-254, ERAS-4001, HB-700 (G12X+G13D), JAB-23400, OC211, PF-07934040, QTX3034, RSC-1255, YL-17231 or ZG2001. In some embodiments, the Ras inhibitor is JAB-23400. In some embodiments, the Ras inhibitor is BI-2852. In some embodiments, the Ras inhibitor targets both the ON and OFF states of Ras, such as BBO-11818, JAB-23E73, JAB-23425, JAB-23E73, BBO-8520, FMC-376, GFH375 (VS-7375), INCB161734, QTX3046, TSN1611, or TH-Z835.

In some embodiments, reference to the term RAS (OFF) inhibitor includes any such RAS (OFF) inhibitor disclosed in any one of the following patent applications: WO 2024206766, WO 2024206747, WO 2024197503, WO 2024193698, WO 2024192424, WO 2024179546, WO 2024178313, WO 2024178304, WO 2024173842, WO 2024167922, WO 2024160225, WO 2024159471, WO 2024159470, WO 2024158778, WO 2024158242, WO 2024153119, WO 2024153116, WO 2024138486, WO 2024138206, WO 2024138052, WO 2024131829, WO 2024125642, WO 2024125600, WO 2024123913, WO 2024123102, WO 2024120433, WO 2024120419, WO 2024119277, WO 2024118926, WO 2024109233, WO 2024112654, WO 2024104453, WO 2024104425, WO 2024107686, WO 2024104453, WO 2024103010, WO 2024097559, WO 2024091409, WO 2024088273, WO 2024085661, WO 2024083258, WO 2024083256, WO 2024083246, WO 2024083168, WO 2024078555, WO 2024076674, WO 2024076672, WO 2024076670, WO 2024067714, WO 2024067575, WO 2024064335, WO 2024063578, WO 2024063576, WO 2024061370, WO 2024061333, WO 2024061267, WO 2024056063, WO 2024055112, WO 2024054926, WO 2024054647, WO 2024054625, WO 2024051763, WO 2024051721, WO 2024050742, WO 2024050640, WO 2024046406, WO 2024046370, WO 2024045066, WO 2024044667, WO 2024044649, WO 2024044334, WO 2024041621, WO 2024041606, WO 2024041589, WO 2024041573, WO 2024040131, WO 2024040109, WO 2024040080, WO 2024036270, WO 2024034657, WO 2024034593, WO 2024034591, WO 2024034123, WO 2024032747, WO 2024032704, WO 2024032703, WO 2024032702, WO 2024031088, WO 2024030647, WO 2024030633, WO 2024029613, WO 2024022507, WO 2024022444, WO 2024020159, WO 2024019103, WO 2024017859, WO 2024017392, WO 2024015731, WO 2024015262, WO 2024012456, WO 2024009191, WO 2024008179, WO 2024008178, WO 2024008068, WO 2024006445, WO 2024006424, WO 2024002373, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023246914, WO 2023246903, WO 2023246777, WO 2023244713, WO 2023244615, WO 2023244604, WO 2023244600, WO 2023244599, WO 2023230190, WO 2023226630, WO 2023225302, WO 2023225252, WO 2023220421, WO 2023219941, WO 2023217148, WO 2023215802, WO 2023215801, WO 2023213269, WO 2023212548, WO 2023208005, WO 2023205719, WO 2023199180, WO 2023198191, WO 2023197984, WO 2023190748, WO 2023185864, WO 2023183755, WO 2023183585, WO 2023179703, WO 2023179629, WO 2023173017, WO 2023173016, WO 2023173014, WO 2023172737, WO 2023171781, WO 2023159087, WO 2023159086, WO 2023154766, WO 2023152255, WO 2023151674, WO 2023151621, WO 2023150394, WO 2023150284, WO 2023143623, WO 2023143605, WO 2023143352, WO 2023143352, WO 2023143312, WO 2023141570, WO 2023141300, WO 2023138662, WO 2023138601, WO 2023138589, WO 2023138524, WO 2023133183, WO 2023133181, WO 2023130012, WO 2023125989, WO 2023125627, WO 2023122662, WO 2023122154, WO 2023120742, WO 2023119677, WO 2023117681, WO 2023116934, WO 2023116895, WO 2023114733, WO 2023105491, WO 2023104018, WO 2023103906, WO 2023103523, WO 2023101928, WO 2023099624, WO 2023099624, WO 2023099620, WO 2023099612, WO 2023099608, WO 2023099592, WO 2023098832, WO 2023098425, WO 2023097227, WO 2023081840, WO 2023081476, WO 2023078424, WO 2023077441, WO 2023072297, WO 2023072188, WO 2023066371, WO 2023064857, WO 2023061463, WO 2023061294, WO 2023057985, WO 2023056951, WO 2023056421, WO 2023051586, WO 2023049697, WO 2023046135, WO 2023045960, WO 2023041059, WO 2023041059, WO 2023040989, WO 2023040513, WO 2023039240, WO 2023039020, WO 2023036282, WO 2023034290, WO 2023030517, WO 2023030495, WO 2023030385, WO 2023030495, WO 2023030517, WO 2023030685, WO 2023030687, WO 2023034290, WO 2023036282, WO 2023039240, WO 203020347, WO 2023025116, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023040989, WO 2023039240, WO 2023039020, WO 2023036282, WO 2023034290, WO 2023030517, WO 2023030495, WO 2023030385, WO 2023025116, WO 2023020523, WO 2023020521, WO 2023020519, WO 2023020518, WO 2023020347, WO 2023018812, WO 2023018810, WO 2023018809, WO 2023018699, WO 2023014979, WO 2023014006, WO 2023004102, WO 2023003417, WO 2023001141, WO 2023001123, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762 (e.g., Compound 44 or Compound 66a), WO 2022212894, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021113595, WO 2021107160, WO 2021106231, WO 2021088458, WO 2021086833, WO 2021085653, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, KR 20240101190, KR 20240101189, KR 20240041720, KR 20240041719, CN 118666870, CN 118666869, CN 118580238, CN 118307563, CN 118221700, CN 118221699, CN 118221698, CN 118221685, CN 118126064, CN 118078802, CN 118078801, CN 118005656, CN 117986263, CN 117986263, CN 117946135, CN 117924327, CN 117903117, CN 117800990, CN 117800989, CN 117800976, CN 117736226, CN 117683051, CN 117645627, CN 117624194, CN 117624190, CN 117586280, CN 117486901, CN 117466917, CN 117462688, CN 117362315, CN 117327102, CN 117327094, CN 117327074, CN 117285590, CN 117263959, CN 117247382, CN 117186095, CN 117164605, CN 116969977, CN 116925075, CN 116891489, CN 116731045, CN 116731044, CN 116554208, CN 116514846, CN 116478184, CN 116478141, CN 116410145, CN 116375742, CN 116354988, CN 116332948, CN 116332938, CN 116327956, CN 116262759, CN 116217592, CN 116199703, CN 116162099, CN 116143806, CN 116143805, CN 116120315, CN 116102559, CN 115960105, CN 115894520, CN 115872979, CN 115850267, CN 115785199, CN 115785124, CN 115724842, CN 115724842, CN 115721720, CN 115716840, CN 115703775, CN 115611923, CN 115611898, CN 115583937, CN 115572278, CN 115557949, CN 115521312, CN 115504976, CN 115490709, CN 115466272, CN 115433183, CN 115433179, CN 115403575, CN 115385938, CN 115385937, CN 115385912, CN 115381786, CN 115368383, CN 115368382, CN 115368381, CN 115353506, CN 115322158, CN 115304623, CN 115304602, CN 115197245, CN 115181106, CN 114989195, CN 114989166, CN 114989147, CN 114920741, CN 114920739, CN 114907387, CN 114874234, CN 114874201, CN 114716436, CN 114716435, CN 114685532, CN 114685460, CN 114591319, CN 114539293, CN 114539286, CN 114539246, CN 114437107, CN 114437084, CN 114409653, CN 114380827, CN 114195804, CN 114195788, CN 114437107, CN 114409653, CN 114380827, CN 114195804, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113321654, CN 113248521, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, CN 109574871, US2024270736, or EP 4389751, each of which is incorporated herein by reference in its entirety, including the RAS compound structures disclosed therein which are specifically incorporated herein by reference.

In some embodiments, reference to the term RAS (OFF) inhibitor refers to a pan-KRAS inhibitor, such as selected from one disclosed in any of the following: WO 2024206766, WO 2024206747, WO 2024192424, WO 2024178313, WO 2024178304, WO 2024173842, WO2024153180, WO 2024119277, WO 2024120433, WO 2024115890, WO 2024112654, WO 2024104453, WO 2024104425, WO 2024107686, WO 2024104453, WO 2024103010, WO 2024085661, WO 2024083246, WO 2024083168, WO 2024067575, WO 2024064335, WO 2024063578, WO 2024063576, WO 2024051852, WO 2024051763, WO 2024046370, WO 2024044667, WO 2024041621, WO 2024041606, WO 2024041589, WO 2024040131, WO 2024040109, WO 2024032747, WO 2024032704, WO 2024032703, WO 2024032702, WO 2024031088, WO 2024030647, WO 2024030633, WO 2024015262, WO 2024009191, WO 2024008068, WO 2024002373, WO 2023287896, WO 2023274324, WO 2023246914, WO 2023246777, WO 2023230190, WO 2023215802, WO 2023215801, WO 2023197984, WO 2023190748, WO 2023183585, WO 2023179703, WO 2023173017, WO 2023173016, WO 2023173014, WO 2023172737, WO 2023154766, WO 2023143352, WO 2023143312, WO 2023138589, WO 2023133183, WO 2023122662, WO 2023114733, WO 2023099624, WO 2023099623, WO 2023099612, WO 2023099608, WO 2023099592, WO 2023097227, WO 2023064857, WO 2023056421, WO 2023049697, WO 2023046135, WO 2023039240, WO 2023034290, WO 2023020523, WO 2023020521, WO 2023020519, WO 2023020518, WO 2023001123, WO 2022271823, WO 2022261210, WO 2022258974, WO 2022256459, WO 2022250170, WO 2022248885, WO 2022228543, WO 2022216762, WO 2022072783, WO 2016161361, KR 20240101190, KR 20240101189, KR 20240041720, KR 20240041719, CN 118221700, CN 118126064, CN 117924327, CN 117946135, CN 117800990, CN 117800989, CN 117683051, CN 117486901, CN 117263959, CN 116969977, or CN 116332948, each of which is incorporated herein by reference in its entirety, including the compound structures disclosed therein. In some embodiments, the combination therapy comprising a compound of the present invention may include one or more additional RAS inhibitors, for example, a pan-KRAS inhibitor. In some embodiments, combination comprising a pan-KRAS inhibitor therapy comprises ERAS-4001. In some embodiments, the pan-KRAS inhibitor, is a pan-KRAS inhibitor in a patent application filed in the name of Medshine Discovery, Inc. In some embodiments, combination comprising a pan-KRAS inhibitor therapy includes BGB-53038, BBO-11818, YL-17231, QTX3034, ABT-200, ADT-1004, AN9025, OC211, JAB-23425, BI-2865, BI-2493, ABREV01, A2A-03, or PF-07934040.

In any embodiment employing a RAS (OFF) inhibitor herein, a RAS (OFF) degrader targeting the OFF state of RAS may be employed. These degraders are known in the art. RAS degraders may be found, for example, in one or more of the following applications: WO 2024199266, WO 2024188281, WO 2024/159164, WO 2024/152247, WO 2024/149214, WO 2024131777, WO 2024120424, WO 2024119278, WO 2024118966, WO 2024118960, WO 2024083258, WO 2024083256, WO 2024055112, WO 2024054625, WO 2024050742, WO 2024044334, WO 2024040080, WO 2024034657, WO 2024034593, WO 2024034591, WO 2024034123, WO 2024029613, WO 2024020159, WO 2024019103, WO 2024017392, WO 2023215906, WO 2023185864, WO 2023171781, WO 2023141570, WO 2023138524, WO 2023130012, WO 2023116934, WO 2023099620, WO 2023081476, WO 2023077441, WO 2022260482, CN 118725012, CN 118496502, CN 118496300, CN 118126040, or CN 115785199, each of which is incorporated herein by reference in its entirety.

In some embodiments, the RAS (OFF) inhibitor is a peptide-based inhibitor. Peptide-based RAS (OFF) inhibitors have been developed that target specific regions of the RAS protein, such as the Switch II region or the RAS-effector interface. Non-limiting examples include the K-Ras-binding peptide (Krpep-2d), the Ras inhibitory peptide (RasIn) and LUNA18 (NCT05012618). Peptide-based RAS (OFF) inhibitors are a class of compounds that target the RAS protein by disrupting its interaction with its downstream effectors or other signaling proteins. These inhibitors are typically designed to mimic the binding motifs of RAS-interacting proteins or other RAS effectors, such as RAF or PI3K. By binding to RAS at the same site as these effectors, peptide-based inhibitors can effectively compete with these proteins and prevent the activation of downstream signaling pathways. See, e.g., WO 2024176153, WO 2024101402, WO 2024101386, WO 2023214576, WO 2023140329, WO 2022234853, WO 2022234852, WO 2022234851, and WO 2022234639, each of which is incorporated herein by reference in its entirety.

Peptide-based RAS (OFF) inhibitors can be further classified into two main categories: those that target the RAS-effector interface, and those that target other regions of the RAS protein. Peptide-based inhibitors that target the RAS-effector interface are designed to bind to the switch regions of RAS that are critical for its interaction with downstream effectors, such as RAF or PI3K. These inhibitors typically contain amino acid residues that are similar to those found in the binding motifs of RAS-interacting proteins or effectors and are often designed to form hydrogen bonds or other interactions with key residues on the surface of RAS.

Peptide-based RAS (OFF) inhibitors that target other regions of the RAS protein are typically designed to disrupt other interactions that are critical for the activation or signaling of RAS. For example, some peptide-based inhibitors are designed to bind to the hypervariable region of RAS, which is thought to play a role in membrane localization and anchoring of the protein. By binding to this region, peptide-based inhibitors can prevent the proper localization of RAS to the plasma membrane, which is necessary for its activation and signaling.

Several common motifs have been identified as important for the binding of RAS-interacting proteins and effectors and are often used in the design of peptide-based inhibitors. One example is the RAF-binding domain (RBD), which is found in many RAS-interacting proteins and is important for the interaction of RAS with downstream effectors such as RAF. The RBD contains a conserved amino acid sequence (Arg-Xaa-Arg) that is critical for binding to RAS, and this motif has been incorporated into several peptide-based inhibitors designed to disrupt the RAS-RAF interaction. Another example is the RAS-binding domain (RBD) of PI3K, which is important for the interaction of RAS with this downstream effector. The RBD of PI3K contains several conserved amino acid residues (such as Arg-Arg-Trp) that are critical for binding to RAS, and these motifs have been used in the design of peptide-based inhibitors that target the RAS-PI3K interaction. Other common motifs used in peptide-based RAS (OFF) inhibitors include the Ras-binding domain (RBD) of other RAS-interacting proteins such as RaIGDS and SOS, as well as sequences that mimic the structure of the switch regions of RAS itself. These motifs are typically used to optimize the binding affinity and selectivity of the inhibitor for the desired target protein or interaction.

In some embodiments, the RAS (OFF) inhibitor is an antibody or antigenic binding peptide specific for RAS (OFF). Antibodies have been developed that bind to specific regions of the RAS protein, such as the Switch II region or the RAS-effector interface. For example, some antibodies have been developed that target the switch regions of RAS proteins, which are critical for the activation of these proteins and their interaction with downstream effectors. Binding of these antibodies to the switch regions can prevent the conformational changes required for RAS activation and downstream signaling. Another approach involves the use of antibodies that target RAS-interacting proteins or downstream effectors, such as RAF or PI3K. Binding of these antibodies to their target proteins can disrupt the RAS-dependent signaling pathways and inhibit the growth and survival of cancer cells. Additionally, some antibodies have been developed that can induce the internalization and degradation of RAS proteins, leading to their depletion and inhibition of downstream signaling. For example, some antibodies have been developed that recognize the unique structure of mutant RAS proteins and target them for degradation via the ubiquitin-proteasome pathway. Non-limiting examples of KRAS (OFF)-specific inhibitory antibodies include anti-p21ser, and K27 (DARPin) (see, e.g., Khan et al, Biochim Biophys Acta Mol Cell Res. 2020 February; 1867 (2): 118570). See also WO 2024136608 and WO 2024111590, each of which is incorporated herein by reference in its entirety.

Antibody-drug conjugates may also be constructed using RAS inhibitors, such as WO 2024189481, which is incorporated herein by reference in its entirety, including the compound structures disclosed therein.

### ii) SOS1 Inhibitors

In some embodiments, compositions and methods described herein may include a compound of the present invention in combination with one or more SOS1 inhibitors. A SOS1 inhibitor may be administered or formulated in combination with compound of the present invention and/or any additional therapeutic agent described herein. In some embodiments, a SOS1 inhibitor is one or more of RMC-5845, RMC-4948, RMC-0331, BI-1701963, BI-3406, SDR5, MRTX-0902, ZG2001, and BAY-293. In some embodiments, reference to the term SOS1 inhibitor includes any such SOS1 inhibitor disclosed in any one of the following patent applications: WO 2023109929, WO 2023059597, WO 2023029833, WO 2023041049, WO 2023022497, WO 2022157629, WO 2022184116, WO 2022170952, WO 2022170917, WO 2022171184, WO 2022170802, WO 2022161461, WO 2022121813, WO 2022028506, WO 2022139304, WO 2021228028, WO 2019122129, CN 115215847, CN 115028644, CN 114685488, CN 111393519, CN 115677702, and CN 115806560 each of which is incorporated herein by reference in its entirety, including the compound structures disclosed therein which are specifically incorporated herein by reference.

iii) SHP Inhibitors

In some embodiments, compositions and methods described herein may include a compound of the present invention in combination with one or more SHP inhibitors. A SHP inhibitor may be administered or formulated in combination with a compound of the present invention and/or any additional therapeutic agent described herein. In some embodiments, the SHP inhibitor is an inhibitor of SHP1. In some embodiments, the SHP inhibitor is an inhibitor of SHP2. In some embodiments, the SHP1 inhibitor is SB8091 or SB6299 aka DA-4511. In some embodiments, a SHP2 inhibitor is one or more of SHP099, TNO155, RMC-4550, RMC-4630, JAB-3068, JAB-3312, RLY-1971, ERAS-601, SH3809, PF-07284892, or BBP-398. In some embodiments, reference to the term SHP2 inhibitor includes any such SHP2 inhibitor disclosed in any one of the following patent applications: WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2022063190, WO 2022043865, WO 2022042331, WO 2022033430, WO 2022017444, WO 2022007869, WO 2021259077, WO 2021249449, WO 2021249057, WO 2021244659, WO 2021218755, WO 2021176072, WO 2021171261, WO 2021149817, WO 2021148010, WO 2021147879, WO 2021143823, WO 2021143701, WO 2021143680, WO 2021281752, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Pat. Nos. 11,179,397, 11,044,675, 11,034,705, 11,033,547, 11,001,561, 10,988,466, 10,954,243, 10,934,302, or U.S. Pat. No. 10,858,359, each of which is incorporated herein by reference in its entirety, including the compound structures disclosed therein which are specifically incorporated herein by reference.

### iv) MEK Inhibitors

In some embodiments, compositions and methods described herein may include a compound of the present invention in combination with one or more MEK inhibitors. A MEK inhibitor may be administered or formulated in combination with a compound of the present invention and/or any additional therapeutic agent described herein. In some embodiments, a MEK inhibitor is one or more of pimasertib, IMM-1-104, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from AE51-Q58; AF53-Q58; E203K; L177M; C121S; F53L; K57E; Q56P; and K57N. In some embodiments, reference to the term MEK inhibitor includes any such MEK inhibitor disclosed in any one of the following patent applications: WO 2022221866, WO 2022125941, WO 2022208391, WO 2022015736, WO 2022177557, WO 2021018866, WO 2021069486, WO 2021142144, WO 2021168283, WO 2021234097, WO 2019076947, WO 2018233696, WO 2016188472, WO 2014063024, WO 2013019906, WO 2011047238, WO 2007044515, US2023032403, and CN 115813930, each of which is incorporated herein by reference in its entirety, including the compound structures disclosed therein which are specifically incorporated herein by reference.

### v) RAF Inhibitors

In some embodiments, compositions and methods described herein may include a compound of the present invention in combination with one or more RAF inhibitors. A RAF inhibitor may be administered or formulated in combination with a compound of the present invention and/or any additional therapeutic agent described herein. In some embodiments, a RAF inhibitor is VS-6766 or BTDX-4933. In some embodiments, a RAF inhibitor is a BRAF inhibitor. BRAF inhibitors that may be used in combination with a compound of the present invention include, for example, Vs6766, IK-595, vemurafenib, dabrafenib, and encorafenib. BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N5811; D594N; D594G; D594A; D594H; F595L; G596D; G596R and A762E. In some embodiments, reference to the term RAF inhibitor includes any such RAF inhibitor disclosed in any one of the following patent applications: WO 2023076991, WO 2022226626, WO 2022226261, WO 2019084459, WO 2018203219, WO 201851306, WO 2017212442, WO 2015075483, WO 2013134243, WO 2013134298, WO 2011047238, WO 2011025965, WO 2011025947, WO 2011025951, WO 2011025940, WO 2011025938, WO 2010065893, WO 2009016460, WO 2009130015, WO 2009111278, WO 2009111279, WO 2008028141, and WO 2006024834, each of which is incorporated herein by reference in its entirety, including the compound structures disclosed therein which are specifically incorporated herein by reference.

### vi) ERK Inhibitors

In some embodiments, compositions and methods described herein may include a compound of the present invention in combination with one or more ERK inhibitors. An ERK inhibitor may be administered or formulated in combination with a compound of the present invention and/or any additional therapeutic agent described herein. In some embodiments, an ERK inhibitor is an ERK1/2 inhibitor, such as ERAS-007. In some embodiments, an ERK inhibitor is an ERK 5 inhibitor. In some embodiments, an ERK inhibitor is one or more of ASTX-029 or 1-75. In some embodiments, reference to the term ERK inhibitor includes any such ERK inhibitor disclosed in any one of the following patent applications: WO 2023076305, WO 2022259222, WO 2022221547, WO 2021110169, WO 2021110168, WO 2021252316, WO 2020102686, WO 2020228817, WO 2020107987, WO 2019233456, WO 2019233457, WO 2016025561, WO 2016192063, WO 2016106029, WO 2016106009, WO 2015051341, WO 2014124230, WO 2014052563, WO 2011041152, WO 200910550, WO 2008153858, CN 114315837, CN 115057860, CN 107973783, each of which is incorporated herein by reference in its entirety, including the compound structures disclosed therein which are specifically incorporated herein by reference.

vii) MAPK Inhibitors

In some embodiments, compositions and methods described herein may include a compound of the present invention in combination with one or more Mitogen-Activated Protein Kinase (MAPK) inhibitors. A MAPK inhibitor may be administered or formulated in combination with a compound of the present invention and/or any additional therapeutic agent described herein. In some embodiments, a MAPK inhibitor is a p38MAPK inhibitor or a MAP3K8 inhibitor. In some embodiments, the MAPK inhibitor is one or more of Tilpisertib (GS-4875) and neflamapidmod (VX-745). In some embodiments, reference to the term MAPK inhibitor includes any such MAPK inhibitor disclosed in any one of the following patent applications: WO 2016029263, CN 114767674, CN 115850179, and CN 1743006, each of which is incorporated herein by reference in its entirety, including the compound structures disclosed therein which are specifically incorporated herein by reference.

In some embodiments, a therapeutic agent that may be combined with a compound of the present invention is an inhibitor of MAP2K4. A non-limiting example of a MAP2K4 inhibitor useful according to the disclosure is HRX-0233.

### c) Kinase Inhibitors

Compositions and methods described herein may include a compound of the present invention in combination with one or more kinase inhibitors. Tyrosine kinases and serine/threonine kinases play a crucial role in various cellular processes such as cell signaling, growth, and differentiation. Kinase inhibitors known in the art have been developed as a treatment for various types of cancer in addition to therapies for conditions such as neurodegenerative diseases, autoimmune disorders, and inflammation.

### i) PKA Inhibitors

In some embodiments, compositions and methods described herein may include one or more Protein Kinase A (PKA) inhibitors. A PKA inhibitor may be administered or formulated in combination with a compound of the present invention and/or any additional therapeutic agent described herein. In some embodiments, a PKA inhibitor is H89. In some embodiments, reference to the term PKA inhibitor includes any such PKA inhibitor disclosed in any one of the following patent applications: CN 106620678 and CN 114632155, each of which is incorporated herein by reference in its entirety, including the compound structures disclosed therein which are specifically incorporated herein by reference.

### ii) FAK Inhibitors

In some embodiments, compositions and methods described herein may include a compound of the present invention in combination with one or more Focal Adhesion Kinase (FAK) inhibitors. A FAK inhibitor may be administered or formulated in combination with a compound of the present invention and/or any additional therapeutic agent described herein. In some embodiments, a FAK inhibitor is one or more of BI853520, defactinib, GSK2256098, PF-00562271, and VS-4718. In some embodiments, reference to the term FAK inhibitor includes any such FAK inhibitor disclosed in any one of the following patent applications: WO 2022152315, WO 2021098679, WO 2020135442, WO 2020191448, WO 2012022408, WO 2013134353, WO 2012110774, WO 2010062578, CN 111072571, and KR 101691536, each of which is incorporated herein by reference in its entirety, including the compound structures disclosed therein which are specifically incorporated herein by reference.

iii) ROCK Inhibitors

In some embodiments, compositions and methods described herein may include a compound of the present invention in combination with one or more Rho-associated, coiled-coil containing protein kinase (ROCK) inhibitors. A ROCK inhibitor may be administered or formulated in combination with a compound of the present invention and/or any additional therapeutic agent described herein. In some embodiments, a ROCK inhibitor is GSK269962A. In some embodiments, reference to the term ROCK inhibitor includes any such ROCK inhibitor disclosed in any one of the following patent applications: WO 2023051753, WO 2022237892, WO 2022012409, WO 2021093795, WO 2021214200, WO 2020177292, WO 202011751, WO 2019014304, WO 2019179525, WO 2019089868, WO 2019014300, WO 2018108156, WO 2018009627, WO 2018009625, WO 2018009622, WO 2017123860, WO 2017205709, WO 2016112236, WO 2014068035, WO 2013030367, WO 2012146724, WO 2012067965, WO 2011107608, CN 108129453, CN 108191821, CN 110917352, CN 108558823, CN 108047193, CN 107973777, CN 108047197, CN 108129448, CN 115869304, and GB202214708, each of which is incorporated herein by reference in its entirety, including the compound structures disclosed therein which are specifically incorporated herein by reference.

### iv) MSK1 Inhibitors

In some embodiments, compositions and methods described herein may include a compound of the present invention in combination with one or more Mitogen- and stress-activated kinase (MSK1) inhibitors. A MSK1 inhibitor may be administered or formulated in combination with a compound of the present invention and/or any additional therapeutic agent described herein. In some embodiments, a MSK1 inhibitor is one or more of SB-747651A, SB 747651A, Ro 320432, CGP 57380, GSK2830371, SR1664, LY-3214996, PFI-4, MSC-2363318A, and AS601245.

### v) RSK Inhibitors

In some embodiments, compositions and methods described herein may include a compound of the present invention in combination with one or more ribosomal S6 kinase (RSK) inhibitors. A RSK1 inhibitor may be administered or formulated in combination with a compound of the present invention and/or any additional therapeutic agent described herein. In some embodiments, a RSK inhibitor is one or more of BI-D1870, LJH685, SL0101-1, FMK, BRD7389, BIX 02565, LJI308, LJI308-S, LJI308-1, and LJH685-S. In some embodiments, a RSK inhibitor is PMD-026. In some embodiments, reference to the term RSK inhibitor includes any such RSK inhibitor disclosed in any one of the following patent applications: WO 2021249558, WO 2020165646, WO 2017141116, and CN 113801139, each of which is incorporated herein by reference in its entirety, including the compound structures disclosed therein which are specifically incorporated herein by reference.

### vi) ALK Inhibitors

In some embodiments, compositions and methods described herein may include a compound of the present invention in combination with one or more Anaplastic Lymphoma Kinase (ALK) inhibitors. An ALK inhibitor may be administered or formulated in combination with a compound of the present invention and/or any additional therapeutic agent described herein. In some embodiments, an ALK inhibitor is one or more of Crizotinib (Xalkori), Ceritinib (Zykadia), Alectinib (Alecensa), Brigatinib (Alunbrig), Lorlatinib (Lorbrena), Ensartinib (X-396), TAE684, ASP3026, TPX-0131, LDK378 (Ceritinib analog), CEP-37440; 4SC-203, TL-398, PLB1003, TSR-011, CT-707, TPX-0005, and AP26113. Additional examples of ALK kinase inhibitors are described in examples 3-39 of WO05016894. In some embodiments, reference to the term ALK inhibitor includes any such ALK inhibitor disclosed in any one of the following patent applications: WO 2019142095, WO 2019179482, WO 2018130928, WO 2018127184, WO 2017101803, WO 2016192132, WO 2014100431, WO 2012082972, CN 111138492, CN 110526914, CN 109836415, CN 105801603, CN 107987056, and CN 105878248, each of which is incorporated herein by reference in its entirety, including the compound structures disclosed therein which are specifically incorporated herein by reference.

### d) Receptor Tyrosine Kinase Inhibitors

Compositions and methods described herein may include a compound of the present invention in combination with one or more receptor tyrosine kinase inhibitors. A receptor tyrosine kinase (RTK) inhibitor is a type of molecule (e.g., small molecule, antibody, and nucleic acid) that binds to and blocks the activity of receptor tyrosine kinases or their ligands. RTKs are proteins found on the surface of cells that play a critical role in cell signaling and growth and have been developed as therapeutics for a range of diseases, including cancer, diabetes, and autoimmune disorders. In some embodiments, a therapeutic agent may be a pan-RTK inhibitor, such as afatinib.

i) EGFR inhibitors

In some embodiments, compositions and methods described herein may include a compound of the present invention in combination with one or more EGFR inhibitors. An EGFR inhibitor may be administered or formulated in combination with a compound of the present invention and/or any additional therapeutic agent described herein. EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15: 59 (8): 1935-40; and Yang et al., Cancer Res. 1999, 59:1236-1243. The EGFR inhibitor can be monoclonal antibody Mab E7.6.3 (Yang, 1999 supra), or Mab C₂₂₅ (ATCC Accession No. HB-8508), or an antibody or antibody fragment having the binding specificity thereof.

Small molecule antagonists of EGFR include gefitinib (Iressa®), Lazertinib, erlotinib (Tarceva®), and lapatinib (TykerBR). See, e.g., Yan et al., Pharmacogenetics and Pharmacogenomics In Oncology Therapeutic Antibody Development, BioTechniques 2005, 39 (4): 565-8; and Paez et al., EGFR Mutations In Lung Cancer Correlation With Clinical Response To Gefitinib Therapy, Science 2004, 304 (5676): 1497-500. In some embodiments, the EGFR inhibitor is osimertinib (Tagrisso®). In some embodiments, an EGFR inhibitor is one or more of cetuximab, gefitinib (Iressa), erlotinib (Tarceva), and afatinib (Gilotrif). Additional non-limiting examples of small molecule EGFR inhibitors include any of the EGFR inhibitors described in Traxler et al., Exp. Opin. Ther. Patents 1998, 8 (12): 1599-1625. An EGFR inhibitor may be ERAS-801. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4). In some embodiments, the EGFR inhibitor may be bosutinib, crizotinib, dasatinib, erlotinib, gefitinib, lapatinib, pazopanib, ruxolitinib, sunitinib, vemurafenib, abrocitinib, asciminib, futibatinib, ibrutinib, imatinib, pacritinib, or sorafenib. In some embodiments, reference to the term EGFR inhibitor includes any such EGFR inhibitor disclosed in any one of the following patent applications: WO 2023041071, WO 2023049312, WO 2023020600, WO 2023284747, WO 2022206797, WO 2022258977, WO 2022033416, WO 2022033410, WO 2022105908, WO 2022100641, WO 2022014639, WO 2022007841, WO 2021018009, WO 2021057882, WO 2021252661, WO 2021018003, WO 2021073498, WO 2021238827, WO 2020254547, WO 2020216371, WO 2020147838, WO 2020207483, WO 2020254572, WO 2020001350, WO 2021001351, WO 2019164948, WO 2019218958, WO 2019046775, WO 2019015655, WO 2018121758, WO 2018218963, WO 2017220007, WO 2017205459, WO 2017161937, WO 2016192609, WO 199633980, WO 199630347, WO 199730034, WO 199730044, WO 199738994, WO 199749688, WO 199802434, WO 199738983, WO 199519774, WO 199519970, WO 199713771, WO 199802437, WO 199802438, WO 199732881, WO 199833798, WO 199732880, WO 199732880, WO 199702266, WO 199727199, WO 199807726, WO 1997/34895, WO 199631510, WO 199814449, WO 199814450, WO 199814451, WO 199509847, WO 199719065, WO 199817662, WO 199935146, WO 199935132, WO 199907701, WO 199220642, DE 19629652, EP 682027, EP 837063, EP 0787772, EP 0520722, EP 0566226, CN 115960018, CN 110283162, CN 114044774, CN 111973601, CN 111973602, and CN 113896744, each of which is incorporated herein by reference in its entirety, including the compound structures disclosed therein which are specifically incorporated herein by reference.

### ii) HER2 Inhibitors

In some embodiments, compositions and methods described herein may include a compound of the present invention in combination with one or more HER2 inhibitors. A HER2 inhibitor may be administered or formulated in combination with a compound of the present invention and/or any additional therapeutic agent described herein. In some embodiments, an HER2 inhibitor is one or more of tucatinib, rastuzumab (Herceptin), pertuzumab (Perjeta), lapatinib (Tykerb), ado-trastuzumab emtansine (Kadcyla), and neratinib (Nerlynx). Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327. In some embodiments, reference to the term HER2 inhibitor includes any such HER2 inhibitor disclosed in any one of the following patent applications: WO 2021156178, WO 2021156180, WO 2021213800, WO 2021088987, WO 2013561183, and WO 2013056108, each of which is incorporated herein by reference in its entirety, including the compound structures disclosed therein which are specifically incorporated herein by reference.

iii) MET Inhibitors

In some embodiments, compositions and methods described herein may include a compound of the present invention in combination with one or more MET inhibitors. A MET inhibitor may be administered or formulated in combination with a compound of the present invention and/or any additional therapeutic agent described herein. In some embodiments, a MET inhibitor is one or more of Crizotinib (Xalkori), Cabozantinib (Cometriq, Cabometyx), Capmatinib (Tabrecta), Tepotinib (Tepmetko), Savolitinib (Volitinib), Onartuzumab (MetMab), Foretinib (GSK1363089), MGCD-265 (Amuvatinib), SU11274, and SU5416. In some embodiments, reference to the term MET inhibitor includes any such MET inhibitor disclosed in any one of the following patent applications: WO 2022226168, WO 2021222045, WO 2020047184, WO 2020015744, WO 2020244654, WO 2020156453, WO 2019206268, WO 2018077227, WO 2017012539, WO 2016015653, WO 2016012963, WO 2012015677, WO 2011162835, WO 2010089507, WO 2009091374, WO 2009056692, WO 2008051547, WO 2007130468, US2012237524, CN 103497177, CN 107311983, CN 107382968, CN 110218191, and TW201331206, each of which is incorporated herein by reference in its entirety, including the compound structures disclosed therein which are specifically incorporated herein by reference.

### iv) AXL Inhibitors

In some embodiments, compositions and methods described herein may include a compound of the present invention in combination with one or more AXL inhibitors. An AXL inhibitor may be administered or formulated in combination with a compound of the present invention and/or any additional therapeutic agent described herein. AXL is a receptor tyrosine kinase that belongs to the TAM family of receptors, which also includes TYRO3 and MERTK. In some embodiments, an AXL inhibitor is one or more of bemcentib, BGB324, R428, SGI-7079, TP-0903, BMS-777607, UNC2025, and TP-0903. In some embodiments, reference to the term AXL inhibitor includes any such AXL inhibitor disclosed in any one of the following patent applications: WO 2023045816, WO 2022237843, WO 2022246179, WO 2021012717, WO 2021088787, WO 2021067772, WO 2021239133, WO 2021204713, WO 2020238802, WO 2019039525, WO 2019101178, WO 2019074116, WO 2017146236, WO 2016097918, WO 2015012298, WO 2010005876, WO 2010083465, CN 115073367, and JP 2022171109, each of which is incorporated herein by reference in its entirety, including the compound structures disclosed therein which are specifically incorporated herein by reference.

### v) IGFR Inhibitors

In some embodiments, compositions and methods described herein may include a compound of the present invention in combination with one or more insulin-like growth factor receptor 1 (IGF-1R) inhibitors. An IGFR inhibitor may be administered or formulated in combination with a compound of the present invention and/or any additional therapeutic agent described herein. IGFR inhibitors have been developed to target the IGFR receptor, which plays a critical role in cancer progression and metastasis. In some embodiments, an IGFR inhibitor is one or more of linsitinib, AXL1717, OSI-906 (Linsitinib), BMS-754807, BI 836845, AZ12253801, PQIP (Pyrrolo[1,2-a]quinoxaline), and NVP-AEW541. In some embodiments, reference to the term IGFR inhibitor includes any such IGFR inhibitor disclosed in any one of the following patent applications: WO 2022115946, WO 2022217923, WO 2021203861, WO 2021246413, WO 2020116398, WO 2019046600, WO 2018195250, WO 2018221521, WO 2018204872, WO 2017072196, WO 2016173682, WO 2015162291, WO 2015162292, WO 2010066868, WO 2006069202, and CN 112125916, each of which is incorporated herein by reference in its entirety, including the compound structures disclosed therein which are specifically incorporated herein by reference.

### vi) RET Inhibitors

In some embodiments, compositions and methods described herein may include a compound of the present invention in combination with one or more Rearranged during transfection (RET) inhibitors. An RET inhibitor may be administered or formulated in combination with a compound of the present invention and/or any additional therapeutic agent described herein. RET plays a critical role in various cellular processes, including cell growth, differentiation, survival, and migration. RET is activated by binding of its ligands, such as glial cell line-derived neurotrophic factor (GDNF) family ligands, which leads to the activation of downstream signaling pathways that promote these cellular processes. In some embodiments, a RET inhibitor is one or more of pralsetinib, selpercatinib (LOXO-292), BLU-667, RXDX-105, TPX-0046, GSK3179106, molidustat (BAY 85-3934), and RPI-1 (Retrophin). In some embodiments, reference to the term RET inhibitor includes any such RET inhibitor disclosed in any one of the following patent applications: WO 2021211380, WO 2021057963, WO 2021043209, WO 2021222017, WO 2020035065, WO 2020114487, WO 2020200314, WO 2020200316, WO 2020114494, WO 2018071447, WO 2018213329, WO 2017079140, WO 2014050781, CN 113943285, CN 113683610, CN 113683611, CN 113620944, CN 113620945, CN 113527291, CN 113527292, CN 113527290, CN 113135896, CN 111057075, CN 111233899, and CN 111362923, each of which is incorporated herein by reference in its entirety, including the compound structures disclosed therein which are specifically incorporated herein by reference.

vii) ROS1 Inhibitors

In some embodiments, compositions and methods described herein may include a compound of the present invention in combination with one or more c-ros oncogene 1 (ROS1) inhibitors. A ROS1 inhibitor may be administered or formulated in combination with a a compound of the present invention and/or any additional therapeutic agent described herein. ROS1 is a receptor tyrosine kinase that belongs to the insulin receptor family and plays a role in various cellular processes, including cell growth, differentiation, survival, and migration. In some embodiments, a ROS1 inhibitor is one or more of taletrectinib, DS-6051b, TPX-0131, GZD824, and PF-06463922. In some embodiments, reference to the term ROS1 inhibitor includes any such ROS1 inhibitor disclosed in any one of the following patent applications: WO 2021098703, WO 2020024825, and US2017079972, each of which is incorporated herein by reference in its entirety, including the compound structures disclosed therein which are specifically incorporated herein by reference.

viii) PDGFR Inhibitors

In some embodiments, compositions and methods described herein may include a compound of the present invention in combination with one or more platelet-derived growth factor receptor (PDGFR) inhibitors. A PDGFR inhibitor may be administered or formulated in combination with a compound of the present invention and/or any additional therapeutic agent described herein. PDGFR is a family of receptor tyrosine kinases that consists of two members, PDGFRa and PDGFRB. They are activated by binding to their ligands, such as platelet-derived growth factor (PDGF), which leads to the activation of downstream signaling pathways that promote cell growth, proliferation, and survival. In some embodiments, a PDGFR inhibitor is one or more of CP-673451, imatinib, nintedanib (ofev), sunitinib (sutent), pazopanib (votrient), regorafenib (stivarga), and dasatinib (sprycel).

### ix) FGF Inhibitors

In some embodiments, compositions and methods described herein may include a compound of the present invention in combination with fibroblast growth factor (FGF) inhibitors. An FGF inhibitor may be administered or formulated in combination with a compound of the present invention and/or any additional therapeutic agent described herein. FGFRs are a family of receptor tyrosine kinases that consists of four members, FGFR1-4. FGFRs are activated by binding to their ligands, fibroblast growth factors (FGFs), which leads to the activation of downstream signaling pathways that promote cell growth, differentiation, and survival. In some embodiments, the FGFR inhibitor is an inhibitor of FGFR2. In some embodiments, the FGFR inhibitor is an inhibitor of FGFR4. In some embodiments, an FGFR inhibitor is one or more of futibatinib (TAK-659), erdafitinib (balversa), infigratinib (Truseltiq), Debio 1347, and rogaratinib (BAY 1163877). In some embodiments, reference to the term FGFR inhibitor includes any such FGFR inhibitor disclosed in any one of the following patent applications: WO 2022033472, WO 2022152274, WO 2022166469, WO 2022206939, WO 2021037219, WO 2021089005, WO 2021113462, WO 2020185532, WO 2019213544, WO 2020164603, WO 2019154364, WO 2019034076, WO 2019213506, WO 2019223766, WO 2018028438, WO 2018153373, WO 2018121650, WO 2018010514, WO 2017028816, WO 2017118438, WO 2016134320, WO 2015008844, WO 2014172644, WO 2014007951, WO 2013179033, WO 2013087578, WO 2012047699, CN 105906630, CN 115869315, CN 115141176, CN 115043832, and CN 115028634, each of which is incorporated herein by reference in its entirety. In some embodiments, the FGF pathway inhibitor targets an FGF ligand. Such FGF pathway inhibitors include FGF ligand traps and antibodies. Non-limiting examples include, FP-1039, an FGF ligand trap consisting of the extracellular domain of FGFR1 fused to the Fc portion of human lgG1, designed to sequester FGF ligands and inhibit FGF signaling, and MFGR1877S, a monoclonal antibody targeting FGF ligands, designed to block FGF-mediated signaling, including the compound structures disclosed therein which are specifically incorporated herein by reference.

### x) VEGF Inhibitors

In some embodiments, compositions and methods described herein may include a compound of the present invention in combination with one or more vascular endothelial growth factor (VEGF) signaling inhibitors. VEGF (vascular endothelial growth factor) signaling inhibitors are a class of drugs that target the signaling pathway mediated by VEGF and its receptors. VEGF plays a critical role in angiogenesis, the process of forming new blood vessels from existing ones, and it is overexpressed in many types of cancer, making it an attractive target for cancer therapy. A VEGF inhibitor may be administered or formulated in combination with a compound of the present invention and/or any additional therapeutic agent described herein. In some embodiments, the VEGF inhibitor is an antibody or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto). In some embodiments, the VEGF inhibitor is one or more of bevacizumab, aflibercept, ramucirumab, sorafenib, sunitinib, and pazopanib.

### e) PI3K/mTOR Pathway Inhibitors

Compositions and methods described herein may include a compound of the present invention in combination with one or more inhibitors of the PI3K-AKT-TOR signaling pathway. The PI3K-AKT-mTOR signaling pathway is a critical intracellular pathway that regulates a wide range of cellular processes including cell growth, proliferation, metabolism, and survival. The pathway is initiated when growth factors, such as insulin or IGF-1, bind to cell surface receptors and activate phosphoinositide 3-kinase (PI3K). Activated PI3K then phosphorylates phosphatidylinositol 4,5-bisphosphate (PIP2) to produce phosphatidylinositol 3,4,5-trisphosphate (PIP3), which in turn activates AKT. Activated AKT then phosphorylates a variety of downstream targets including the tuberous sclerosis complex (TSC1/TSC2), leading to the activation of mTOR (mammalian target of rapamycin) complex 1 (mTORC1). Activated mTORC1 promotes protein synthesis and cell growth by phosphorylating key regulators of translation initiation such as S6 kinase (S6K) and eukaryotic initiation factor 4E-binding protein 1 (4E-BP1).

### i) PI3K Inhibitors

In some embodiments, compositions and methods described herein may include a compound of the present invention in combination with one or more PI3K inhibitors. A PI3K inhibitor may be administered or formulated in combination with a compound of the present invention and/or any additional therapeutic agent described herein. PI3K inhibitors include, but are not limited to, wortmannin; 17-hydroxywortmannin analogs described in WO06/044453; 4-[2-(1H-Indazol-4-yl)-6-[4-(methylsulfonyl) piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)—I-(4-((2-(2-aminopyrimidin-5-yl)-7-methyl-4-morpholinothieno[3,2-d]pyrimidin-6-yl)methyl) piperazin-1-yl)-2-hydroxypropan-1-one (described in WO08/070740); LY294002 (2-(4-morpholinyl)-8-phenyl-4H-I-benzopyran-4-one (available from Axon Medchem); PI 103 hydrochloride (3-[4-(4-morpholinylpyrido-[3′,2′: 4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[I-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136. In some embodiments, the PI3K inhibitor is alpelisib or copanlisib.

### i) AKT Inhibitors

In some embodiments, compositions and methods described herein may include a compound of the present invention in combination with one or more AKT inhibitors. An AKT inhibitor may be administered or formulated in combination with a compound of the present invention and/or any additional therapeutic agent described herein. AKT inhibitors include, but are not limited to, ipatasertib, GSK-2141795, Akt-1-1 (inhibits Aktl) (Barnett et al., Biochem. J. 2005, 385 (Pt. 2): 399-408); Akt-1-1,2 (inhibits Akl and 2) (Barnett et al., Biochem. J. 2005, 385 (Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134 (12 Suppl): 3493S-3498S); perifosine (e.g., interferes with Akt membrane localization; Dasmahapatra et al. Clin. Cancer Res. 2004, 10 (15): 5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9). The PI3K/AKT inhibitor may include, but is not limited to, one or more PI3K/AKT inhibitors described in Cancers (Basel) 2015 September; 7 (3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; and GSK2126458.

iii) mTOR Inhibitors

In some embodiments, compositions and methods described herein may include a compound of the present invention in combination with one or more mTOR inhibitors. A mTOR inhibitor may be administered or formulated in combination with a compound of the present invention and/or any additional therapeutic agent described herein. mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl) rapamycin; 40-[3-hydroxy (hydroxymethyl)methylpropanoate]-rapamycin (also known as CC1779); 40-epi-(tetrazolyt)-rapamycin (also called ABT578); 32-deoxorapamycin; 16-pentynyloxy-32 (S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

iv) MNK inhibitors

In some embodiments, compositions and methods described herein may include a compound of the present invention in combination with one or more mitogen-activated protein kinase-interacting kinase (MNK) inhibitors. A MNK inhibitor may be administered or formulated in combination with a compound of the present invention and/or any additional therapeutic agent described herein. MNK proteins are activated downstream of the mitogen-activated protein kinase (MAPK) signaling pathway, which plays a critical role in the regulation of cellular proliferation, differentiation, and survival. MNKs phosphorylate eIF4E, a key component of the eukaryotic translation initiation complex, which enhances the translation of specific mRNAs, including those encoding proteins involved in cell cycle regulation and oncogenesis. In some embodiments, a MNK inhibitor is one or more tomivosertib (eFT508), CGP57380, and SEL201. In some embodiments, reference to the term MNK inhibitor includes any such MNK inhibitor disclosed in any one of the following patent applications: WO 2021098691, WO 2020108619, WO 2020086713, WO 2018152117, WO 2018228275, WO 2015200481, and CN 115583942, each of which is incorporated herein by reference in its entirety, including the compound structures disclosed therein which are specifically incorporated herein by reference.

v) eIF4 Inhibitors

In some embodiments, compositions and methods described herein may include a compound of the present invention in combination with one or more eukaryotic initiation factor 4A (eIF4A) inhibitors. An eIF4A inhibitor may be administered or formulated in combination with a compound of the present invention and/or any additional therapeutic agent described herein. eIF4A is a critical component of the eukaryotic translation initiation complex, where it functions as an RNA helicase to unwind the secondary structure of mRNA and facilitate ribosome binding. eIF4A is required for the translation of many cancer-associated genes, making it an attractive therapeutic target for cancer treatment. In some embodiments, an eIF4A inhibitor is one or more zotatifin (eFT226), silvestrol, pateamine A, and rocaglates. In some embodiments, reference to the term eIF4A inhibitor includes any such eIF4A inhibitor disclosed in any one of the following patent applications: WO 2023034813, WO 2021195128, and WO 2017091585, each of which is incorporated herein by reference in its entirety, including the compound structures disclosed therein which are specifically incorporated herein by reference.

In some embodiments, compositions and methods described herein may include one or more eukaryotic initiation factor 4G (eIF4G) inhibitors. An eIF4G inhibitor may be administered or formulated in combination with a compound of the present invention and/or any additional therapeutic agent described herein. eIF4G family includes several proteins that are involved in the initiation of protein translation. eIF4G serves as a scaffold for other proteins, including eIF4E and eIF4A, to form the eIF4F complex, which is responsible for binding to the 5′ cap of mRNA and unwinding the secondary structure of the mRNA to allow ribosomal scanning and translation initiation. In some embodiments, an eIF4G inhibitor is one or more pateamine A, and hippuristanol.

### f) DNA Damage Response Inhibitors

Compositions and methods described herein may include a compound of the present invention in combination with one or more DNA damage response (DDR) inhibitors. The DDR pathway is a critical cellular pathway that is activated in response to DNA damage and is essential for maintaining genomic stability, thereby preventing the development of cancer. However, cancer cells often have defects in the DDR pathway, which makes them more sensitive to DDR inhibitors. DDR inhibitors have shown promise in preclinical studies as potential cancer therapeutics, particularly in combination with other agents.

### i) Wee1 Inhibitors

In some embodiments, compositions and methods described herein may include a compound of the present invention in combination with one or more Wee1 inhibitors. Wee1 is a kinase that plays a critical role in regulating the cell cycle by inhibiting the activity of cyclin-dependent kinases (CDKs) and preventing the progression of cells through the G2/M checkpoint. Wee1 is overexpressed in several cancer types and has been implicated in tumor growth and survival. In some embodiments, a Wee1 inhibitor is one or more of imp7068, adavosertib, azenosertib or ZNL-02-096. In some embodiments, reference to the term Wee1 inhibitor includes any such Wee1 inhibitor disclosed in any one of the following patent applications: WO 2022011391, WO 2022247641, WO 2021043152, WO 2020221358, WO 2020083404, WO 2020192581, WO 2019085933, WO 2018133829, WO 2015115355, WO 2015183776, WO 2014085216, and CN 114831993, each of which is incorporated herein by reference in its entirety, including the compound structures disclosed therein which are specifically incorporated herein by reference.

### ii) CHK Inhibitors

In some embodiments, compositions and methods described herein may include a compound of the present invention in combination with one or more checkpoint kinase (CHK) inhibitors. A CHK inhibitor may be administered or formulated in combination with a compound of the present invention and/or any additional therapeutic agent described herein. CHK1 kinase is a critical regulator of the cell cycle and the DNA damage response pathway. In some embodiments, the CHK inhibitor is a CHK1 inhibitor. In some embodiments, a CHK inhibitor is a CHK2 inhibitor. In some embodiments, a CHK1 inhibitor is one or more BBI-355, rabusertib, LY2606368, GDC-0575, and MK-8776. In some embodiments, reference to the term CHK1 inhibitor includes any such CHK1 inhibitor disclosed in any one of the following patent applications: WO 2021113661, WO 2021104461, WO 2019012030, WO 2010118390, WO 2008067027, WO 2002070494, and TW202126818, each of which is incorporated herein by reference in its entirety, including the compound structures disclosed therein which are specifically incorporated herein by reference.

iii) ATM Inhibitors

In some embodiments, compositions and methods described herein may include a compound of the present invention in combination with one or more ataxia telangiectasia mutated (ATM) inhibitors. An ATM inhibitor may be administered or formulated in combination with a compound of the present invention and/or any additional therapeutic agent described herein. ATM plays a role in regulating the replication stress response and maintaining genomic stability. In some embodiments, an ATM inhibitor is one or more M4076, AZD0156, KU-60019, and VE-821. In some embodiments, reference to the term ATM inhibitor includes any such ATM inhibitor disclosed in any one of the following patent applications: WO 2021197339, WO 2021098734, WO 2021260580, WO 2007026157, WO 2006085067, and US 2016113935, each of which is incorporated herein by reference in its entirety, including the compound structures disclosed therein which are specifically incorporated herein by reference.

### iv) ATR Inhibitors

In some embodiments, compositions and methods described herein may include a compound of the present invention in combination with one or more ataxia telangiectasia and Rad3-related (ATR) inhibitors. An ATR inhibitor may be administered or formulated in combination with a compound of the present invention and/or any additional therapeutic agent described herein. In some embodiments, an ATR inhibitor is one or more ceralaertib, VE-821, RP-350, AZ20, VX-970, abd110, VX-803, and BAY 1895344. In some embodiments, reference to the term ATR inhibitor includes any such ATR inhibitor disclosed in any one of the following patent applications: WO 2023016529, WO 2022237875, WO 2022268025, WO 2021012049, WO 2021023272, WO 2021260579, WO 2021228758, WO 2019050889, WO 2019154365, WO 2019133711, WO 2017059357, WO 2013049859, WO 2007046426, WO 2007015632, and CN 113797341, each of which is incorporated herein by reference in its entirety, including the compound structures disclosed therein which are specifically incorporated herein by reference.

### v) PARP Inhibitors

In some embodiments, compositions and methods described herein may include a compound of the present invention in combination with one or more Poly(ADP-ribose) polymerase (PARP) inhibitors. A PARP inhibitor may be administered or formulated in combination with a compound of the present invention and/or any additional therapeutic agent described herein. There are 17 PARP (aka tankyrase) family members that have been identified. PARP enzymes play a critical role in DNA damage repair, particularly in the repair of single-strand DNA breaks. PARP inhibitors block the activity of PARP enzymes, leading to the accumulation of DNA damage and ultimately cell death. In some embodiments, a PARP inhibitor is one or more Olaparib, rucaparib, niraparib, and veliparib (ABT-888). In some embodiments, reference to the term PARP inhibitor includes any such PARP inhibitor disclosed in any one of the following patent applications: WO 2023051812, WO 2023051807, WO 2023051716, WO 2023278592, WO 2022228387, WO 2022022664, WO 2022000946, WO 2022222921, WO 2021163530, WO 2020122034, WO 2020239097, WO 2020142583, WO 2020156577, WO 2020098774, WO 2020196712, WO 2019200382, WO 2018125961, WO 2018205938, WO 2018192576, WO 2018218025, WO 2017032289, WO 2017177838, WO 2017029601, WO 2017088723, WO 2016155655, WO 2015154630, WO 2013097225, WO 2012130166, WO 2011006794, WO 2009046205, WO 2009063244, WO 2008084261, WO 2007138351, WO 2006110816, WO 2005053662, WO 2005012524, CN 113698356, CN 113603647, CN 115073544, CN 108938634, CN 104887680, CN 110343088, CN 108976236, and CN 107629071, each of which is incorporated herein by reference in its entirety, including the compound structures disclosed therein which are specifically incorporated herein by reference.

### vi) DNA-PK Inhibitors

In some embodiments, compositions and methods described herein may include a compound of the present invention in combination with one or more DNA-dependent protein kinase (DNA-PK) inhibitors. An DNA-PK inhibitor may be administered or formulated in combination with a compound of the present invention and/or any additional therapeutic agent described herein. DNA-dependent protein kinase (DNA-PK) is a serine/threonine protein kinase that plays a crucial role in DNA repair and maintenance of genome stability. In some embodiments, a DNA-PK inhibitor is one or more NU7441, AZD7648, VX-984, M3814, and CC-115. In some embodiments, reference to the term DNA-PK inhibitor includes any such DNA-PK inhibitor disclosed in any one of the following patent applications: WO 2022187965, WO 2021197159, WO 2021260583, WO 2021204111, WO 2021104277, WO 2021098813, WO 2021022078, WO 2020259613, WO 2019143678, WO 2019143675, WO 2019201283, WO 2015058031, WO 2014159690, WO 2012028233, WO 2009010761, WO 2006032869, WO 2006109084, CN 112574179, CN 112300132, and CN 112300126, each of which is incorporated herein by reference in its entirety, including the compound structures disclosed therein which are specifically incorporated herein by reference.

### g) Cell Cycle Inhibitors

Compositions and methods described herein may include a compound of the present invention in combination with one or more cell cycle inhibitors. Cell cycle inhibitors target specific proteins involved in regulating the cell cycle, which is the process by which a cell divides and replicates its DNA. Non-limiting examples cell cycle proteins include cyclin-dependent kinase (CDK), aurora kinase, and polo-like kinase (PLK). CDKs are a family of kinases that are involved in regulating the cell cycle. CDK inhibitors block the activity of these kinases, leading to cell cycle arrest and/or apoptosis. Aurora kinases are a family of serine/threonine kinases that play a critical role in regulating mitosis. Aurora kinase inhibitors block the activity of these kinases, leading to mitotic arrest and cell death. PLKs are a family of serine/threonine kinases that are involved in regulating multiple stages of the cell cycle. PLK inhibitors block the activity of these kinases, leading to cell cycle arrest and/or apoptosis.

### i) CDK Inhibitors

In some embodiments, compositions and methods described herein may include a compound of the present invention in combination with one or more CDK inhibitors. A CDK inhibitor may be administered or formulated in combination with a compound of the present invention and/or any additional therapeutic agent described herein. Cyclin-dependent kinases are a family of protein kinases that regulate cell division and proliferation. Cell cycle progression is controlled by cyclins and their associated cyclin-dependent kinases, such as CDK1, CDK2, CDK3, CDK4 and CDK6, while other CDKs such as CDK7, CDK8 and CDK9 are critical to transcription. CDK binding to cyclins forms heterodimeric complexes that phosphorylate their substrates on serine and threonine residues, which in turn initiates events required for cell-cycle transcription and progression. In some embodiments, a CDK inhibitor is a CDK2 inhibitor. In some embodiments, a CDK inhibitor is a CDK4/6 inhibitor. In some embodiments, a CDK inhibitor is a CDK7 inhibitor. In some embodiments, a CDK inhibitor is a CDK9 inhibitor. In some embodiments, a CDK inhibitor is one or more palbociclib, ribociclib, abemaciclib, and trilaciclib. In some embodiments, a CDK inhibitor is one or more of tagtociclib (PF-07104091), seliciclib, voruciclib P1446A-05, BLU-222, dinaciclib, AT-7519, RGB286638, and AZD4573.

In some embodiments, reference to the term CDK inhibitor includes any such CDK inhibitor disclosed in any one of the following patent applications: WO 2022166793, WO 2022187611, WO 2022130304, WO 2021227906, WO 2021057867, WO 2020207260, WO 2020138370, WO 2020125513, WO 2020148635, WO 2020215156, WO 2020052627, WO 2017177837, WO 2017162215, WO 2017177836, WO 2016193939, WO 2016014904, WO 2016015598, WO 2016015605, WO 2015181737, WO 2012061156 A1, WO 2012038411, WO 2010020675, WO 2010125004, WO 2007139732, WO 2006024945, CN 114478529, CN 108794496, CN 105294737, CN 107652284, KR 20180106188, and US2017152269, each of which is incorporated herein by reference in its entirety, including the compound structures disclosed therein which are specifically incorporated herein by reference.

### ii) Aurora Kinase Inhibitors

In some embodiments, compositions and methods described herein may include a compound of the present invention in combination with one or more aurora kinase inhibitors. An aurora kinase inhibitor may be administered or formulated in combination with a compound of the present invention and/or any additional therapeutic agent described herein. Aurora kinases are a family of serine/threonine kinases that play a critical role in regulating cell division and maintaining genomic stability. The Aurora kinase family consists of three members: Aurora A, Aurora B, and Aurora C. In some embodiments, an aurora kinase inhibitor is one or more palbociclib, ribociclib, and abemaciclib. In some embodiments, an aurora kinase inhibitor is one or more of alisertib, danusertib, barasertib, and MLN8237. In some embodiments, reference to the term aurora kinase inhibitor includes any such aurora kinase inhibitor disclosed in any one of the following patent applications: WO 2021110009, WO 2021008338, WO 2020112514, WO 2019129234, WO 2016077161, WO 2013143466, WO 2011103089, WO 2010081881, WO 2010133794, WO 2009134658, WO 2008001886, WO 2007095124, WO 2007003596, WO 2006129064, CN 114276227, CN 108078991, CN 106543155, CN 104211692, and CN 104098551, each of which is incorporated herein by reference in its entirety, including the compound structures disclosed therein which are specifically incorporated herein by reference.

iii) PLK Inhibitors

In some embodiments, compositions and methods described herein may include a compound of the present invention in combination with one or more polo-like kinase (PLK) inhibitors. A PLK inhibitor may be administered or formulated in combination with a compound of the present invention and/or any additional therapeutic agent described herein. PLKs are a family of serine/threonine kinases that play a crucial role in regulating cell division, DNA damage response, mitotic progression, and consists of four members: PLK1, PLK2, PLK3, and PLK4. In some embodiments, a PLK inhibitor is one or more of volasertib, onvansertib, BI 2536, and GSK461364. In some embodiments, reference to the term PLK inhibitor includes any such PLK inhibitor disclosed in any one of the following patent applications: WO 2011012534 A1, WO 2010065134, WO 2009130453, WO 2009042806, WO 2004043936, WO 2007030361, WO 2006021547, CN 115804777, and EP 2325185, each of which is incorporated herein by reference in its entirety, including the compound structures disclosed therein which are specifically incorporated herein by reference.

### iv) Kinesin Superfamily of Microtubule Motor Protein Inhibitors

In some embodiments, compositions and methods described herein may include a compound of the present invention in combination with one or more Kinesin spindle protein (KSP) inhibitors. In some embodiments, compositions described herein may include one or more Kinesin family (KIF) inhibitors. In some embodiments, a KSP inhibitor may be administered or formulated in combination with a compound of the present invention and/or any additional therapeutic agent described herein. KSP and KIF are a subset of the kinesin superfamily of microtubule motor proteins. KSP, also known as Eg5, is a member of the kinesin superfamily of motor proteins that plays a critical role in mitotic spindle formation and cell division. KSP inhibitors selectively target rapidly dividing cancer cells by disrupting spindle formation and inducing mitotic arrest. In some embodiments, a KSP inhibitor is one or more of SB743921, monastrol, S-Trityl-L-cysteine (STLC), and filanesib (ARRY-520). In some embodiments, a KIF inhibitor is an inhibitor of a Kinesin-8 family microtubule motor protein. In some embodiments, the kinesin-8 family protein is KIF18A. In some embodiments, a KIF inhibitor is one or more of AMG650, BTB-1, K03861, and SJ000291942. In some embodiments, reference to the term kinesin superfamily of microtubule motor protein inhibitor includes any such kinesin superfamily of microtubule motor protein inhibitor disclosed in any one of the following patent applications: WO 2015114854, WO 2015114855, WO 2010084186, WO 2006101761, WO 2006110390, WO 2006044825, WO 2006078574, WO 2005060654, WO 2004092147, WO 2004037171, WO 2004058700, WO 2003050064, WO 2003105855, WO 2022037665, WO 2018114804, WO 2017162663, WO 2016207089, WO 2012073375, JP 2014162787, JP 2019189590, JP2013166713, and KR 20220145566, each of which is incorporated herein by reference in its entirety, including the compound structures disclosed therein which are specifically incorporated herein by reference.

### v) DYRK1 Inhibitors

In some embodiments, compositions and methods described herein may include a compound of the present invention in combination with one or more Dual-specificity tyrosine phosphorylation-regulated kinase 1 (DYRK1) inhibitors. A DYRK1 inhibitor may be administered or formulated in combination with a compound of the present invention and/or any additional therapeutic agent described herein. DYRK1 is a member of the DYRK (dual-specificity tyrosine phosphorylation-regulated kinase) family of protein kinases. It plays essential roles in various cellular processes, including cell cycle regulation, neuronal development, and transcriptional control. In some embodiments, a DYRK1 inhibitor is one or more of harmine, INDY, D4476, and AZ191. In some embodiments, reference to the term DYRK1 inhibitor includes any such DYRK1 inhibitor disclosed in any one of the following patent applications: WO 2023277331 A1, WO 2023140846 A1, WO 2017181087 A1, each of which is incorporated herein by reference in its entirety, including the compound structures disclosed therein which are specifically incorporated herein by reference.

### h) Anti-Apoptotic Protein Inhibitors

Compositions and methods described herein may include a compound of the present invention in combination with one or more anti-apoptotic protein inhibitors. In some embodiments, an anti-apoptotic protein inhibitor may be administered or formulated in combination with a compound of the present invention and/or any additional therapeutic agent described herein. Anti-apoptotic inhibitors target proteins that play a role in preventing apoptosis, a form of programmed cell death. Apoptosis is a critical mechanism for eliminating damaged or unwanted cells. Anti-apoptotic proteins are a family of proteins that inhibit the apoptotic pathway, thereby preventing cell death. There are several known classes of anti-apoptotic inhibitors, including Bcl-2 inhibitors, XIAP inhibitors, survivin inhibitors, Mcl-1 inhibitors, and FLIP inhibitors. These inhibitors work by binding to specific anti-apoptotic proteins and preventing their activity, thereby promoting cell death in cancer cells. In some embodiments, compositions described herein may include one or more anti-apoptotic protein inhibitors. An anti-apoptotic protein inhibitor may be administered or formulated in combination with a RAS (ON) inhibitor and/or any additional therapeutic agent described herein. In some embodiments, the anti-apoptotic protein inhibitor includes a MCL-1 inhibitor. Non-limiting examples of MCL-1 inhibitors include, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263. In some embodiments, the anti-apoptotic protein inhibitor includes a BCL protein inhibitor. Examples of BCL protein inhibitors include but are not limited to Venetoclax (Venclexta), Navitoclax (ABT-263), A-1331852, S63845, and AT-101.

### i) Autophagy Inhibitors

Compositions and methods described herein may include a compound of the present invention in combination with one or more autophagy inhibitors. In some embodiments, an autophagy inhibitor may be administered or formulated in combination with a compound of the present invention and/or any additional therapeutic agent described herein. Autophagy inhibitors include, but are not limited to chloroquine, 3-methyladenine, hydroxychloroquine (Plaquenil™), spautin-1, SAR405, bafilomycin A1, 5-amino-4-imidazole carboxamide riboside (AICAR), okadaic acid, autophagy-suppressive algal toxins which inhibit protein phosphatases of type 2A or type 1, analogues of CAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

### a) ULK Inhibitors

In some embodiments, compositions and methods described herein may include a compound of the present invention in combination with one or more Unc-51-like kinase (ULK) inhibitors. An ULK inhibitor may be administered or formulated in combination with a compound of the present invention and/or any additional therapeutic agent described herein. In some embodiments, a ULK inhibitor is a ULK1/2 inhibitor. In some embodiments, an ULK inhibitor is one or more of ULK-101, MRT68921, SBI-0206965, MRT67307, MRT68920, MRT68922, MRT199665, LY3009120, and Dorsomorphin.

### b) VPS Inhibitors

In some embodiments, compositions and methods described herein may include a compound of the present invention in combination with one or more Vacuolar protein sorting protein (VPS) inhibitors. A VPS inhibitor may be administered or formulated in combination with a compound of the present invention and/or any additional therapeutic agent described herein. VPS (proteins are a family of proteins that play a critical role in the process of autophagy by regulating the formation and function of autophagosomes, structures that engulf and transport cellular components to lysosomes for degradation. Dysregulation of VPS proteins has been implicated in various diseases, including cancer, neurodegenerative disorders, and infectious diseases. In some embodiments, a VPS inhibitor is a VPS34 inhibitor. In some embodiments, a VPS inhibitor is one or more of PIK—III, VPS34-IN1, SAR405, Spautin-1, and NSC185058.

### c) Macropinocytosis Inhibitors

In some embodiments, compositions and methods described herein may include a compound of the present invention in combination with one or more macropinocytosis inhibitors. A macropinocytosis inhibitor may be administered or formulated in combination with a RAS (ON) inhibitor and/or any additional therapeutic agent described herein. Macropinocytosis inhibitors are compounds that can block or reduce the process of macropinocytosis. In some embodiments, a macropinocytosis inhibitor is one or more of EIPA (ethylisopropylamiloride), Wortmannin, Amiloride, Apilimod, Dyngo-4a, and Latrunculin B.

j) WNT/b-catenin Pathway Inhibitors

Compositions and methods described herein may include a compound of the present invention in combination with one or more WNT/beta-catenin pathway inhibitors. In some embodiments, a WNT/beta-catenin pathway inhibitor may be administered or formulated in combination with a compound of the present invention and/or any additional therapeutic agent described herein. The WNT/beta-catenin pathway is an important signaling pathway that plays a crucial role in development, tissue homeostasis, and disease. Dysregulation of this pathway has been implicated in various cancers, making it an attractive target for cancer therapy. WNT/beta-catenin pathway inhibitors target various components of the pathway, including WNT ligands, receptors, and downstream effectors.

i) b-catenin Inhibitors

In some embodiments, compositions and methods described herein may include a compound of the present invention and one or more b-catenin inhibitors. A b-catenin inhibitor may be administered or formulated in combination with a compound of the present invention and/or any additional therapeutic agent described herein. Beta-catenin is a protein that plays an important role in the WNT signaling pathway, which regulates various cellular processes including cell proliferation, differentiation, and migration. In normal cells, b-catenin levels are tightly regulated by a destruction complex, which marks beta-catenin for degradation. However, in many cancer cells, the destruction complex is impaired, leading to the accumulation of beta-catenin in the nucleus and the activation of target genes involved in tumor growth and metastasis. In some embodiments, a WNT/b-catenin inhibitor is one or more of FOG-001, OMP-131R¹⁰, Foxy-5, LGK974, RXC004, ETC-159, OMP-54F28, Niclosamide, OMP-18R⁵, OTSA-101, BNC101, DKN-01, Sulindac, Pyrvinium, E7449, BC2059, PRI-724, SM08502, IWP1, IWP2, IWP3, IWP4, IWP12, IWP L6, C₅₉, GNF-6231, GNF-1331, DK-520, DK-419, IgG-2919, Fz7-21, RHPD-P1, SRI37892, 1094-0205, 2124-0331, 3235-0367, NSC36784, NSC654259, lgG-2919, Salinomycin, BMD4702, 3289-8625, J01-017a, FJ9, KY-02061, KY-02327, NSC668036, Peptide Pen-N3, SSTC3, CCT031374, TCS 183, XAV939, AZ1366, G007-LK, MSC2504877, G244-LM, IWR-1, JW74, JW55, K-756, NVP-TNKS656, MN-64, RK-287107, WIKI4, KY1220, KYA1797K, MSAB, PKF115-584, CGP049090, AV-65, PNU-74654, Windorphen, IQ-1 tegavivant, foscenvivant, PNPB-29, ZW4864, SAH-BCL9, Carnosic acid, xStAx-VHL, NRX-252114, Septuximab vedotin, PF-06647020, LGR5-mc-vc-PAB-MMAE, LGR5-NMS818, CWP232291, PRI-724 (also known as ICG-001), C-82, and BC2059. In some embodiments, reference to the term b-catenin inhibitor includes any such b-catenin inhibitor disclosed in any one of the following patent applications: CN 104388427 and CN 103830211, each of which is incorporated herein by reference in its entirety, including the compound structures disclosed therein which are specifically incorporated herein by reference.

### ii) PORCN Inhibitors

In some embodiments, compositions and methods described herein may include a compound of the present invention in combination with one or more Porcupine (PORCN) inhibitors. A PORCN inhibitor may be administered or formulated in combination with a compound of the present invention and/or any additional therapeutic agent described herein. PORCN is a membrane-bound O-acyltransferase enzyme that plays a critical role in the WNT signaling pathway by mediating the palmitoylation of WNT ligands. This palmitoylation is essential for the secretion and signaling activity of WMT proteins. Inhibition of PORCN leads to reduced WNT signaling activity. In some embodiments, a PORCN inhibitor is one or more of LGK974 (WNT974), ETC-1922159, CGX1321, and CWP232291.

iii) GSK3 Inhibitors

In some embodiments, compositions and methods described herein may include a compound of the present invention in combination with one or more Glycogen synthase kinase (GSK3) inhibitors. A GSK3 inhibitor may be administered or formulated in combination with a compound of the present invention and/or any additional therapeutic agent described herein. The GSK3 family consists of two closely related serine/threonine kinases: GSK3a and GSK3B. These kinases are involved in numerous cellular processes, including glycogen metabolism, cell cycle regulation, and Wnt signaling. GSK inhibitors have been investigated as potential therapeutics for various diseases, including cancer, diabetes, Alzheimer's disease, and bipolar disorder. In some embodiments, a GSK3 inhibitor is one or more of Tideglusib, laduviglusib, LiCl (Lithium chloride), CHIR99021, SB216763, AZD1080, and LY2090314. In some embodiments, reference to the term GSK3 inhibitor includes any such GSK3 inhibitor disclosed in any one of the following patent applications: WO 2017153834, WO 2014059383, WO 2010012398, WO 2009017455, WO 2003037891, CN 107151235, and CN 102258783, each of which is incorporated herein by reference in its entirety, including the compound structures disclosed therein which are specifically incorporated herein by reference.

### iv) CLK Inhibitors

In some embodiments, compositions and methods described herein may include a compound of the present invention in combination with one or more Cdc2-like kinase (CLK) inhibitors. A CLK inhibitor may be administered or formulated in combination with a compound of the present invention and/or any additional therapeutic agent described herein. LKs (Cdc2-like kinases) are a family of serine/threonine kinases that play a crucial role in pre-mRNA splicing, specifically in the regulation of alternative splicing.

There are four members of the CLK family: CLK1, CLK2, CLK3, and CLK4. The CLK family of kinases have been shown to be involved in several diseases, including cancer, neurodegenerative disorders, and viral infections. In some embodiments, a CLK inhibitor is a CLK 2 inhibitor. In some embodiments, a CLK2 inhibitor is one or more of Lorecivivint, SM08502, SM04690, TG003, KH—CB19, Cmpd-1, T3.5, and CX-4945. In some embodiments, reference to the term CLK inhibitor includes any such CLK inhibitor disclosed in WO 2020006115, which is incorporated herein by reference in its entirety, including the compound structures disclosed therein which are specifically incorporated herein by reference.

### k) JAK/STAT Pathway Inhibitors

Compositions and methods described herein may include a compound of the present invention in combination with one or more JAK/STAT pathway inhibitors. In some embodiments, a JAK/STAT pathway inhibitor may be administered or formulated in combination with a compound of the present invention and/or any additional therapeutic agent described herein. The Janus kinase/signal transducer and activator of transcription (JAK/STAT) pathway is a signaling pathway involved in many cellular processes, including immune response, cell growth, and differentiation. Dysregulation of this pathway has been linked to various diseases, including inflammatory disorders, cancer, and autoimmune diseases. Inhibitors of the JAK/STAT pathway can be used for the treatment of these diseases. In some embodiments, a JAK/STAT pathway inhibitor is an inhibitor of JAK1, JAK2 and/or JAK3. In some embodiments, a JAK inhibitor is one or more of Ruxolitinib (Jakafi@), Pacritinib, Fedratinib, Tofacitinib (Xeljanz@), Abrocitinib, Filgotinib, Oclacitinib, Peficitinib, Upadacitinib, Deucravacitinib, Delgocitinib, and Baricitinib (Olumiant®). In some embodiments, reference to the term JAK inhibitor includes any such JAK inhibitor disclosed in any one of the following patent applications: WO 2023011301, WO 2023201044, WO 2022143629, WO 2022251434, WO 2022067106, WO 2022033551, WO 2021244323, WO 2021238817, WO 2021238818, WO 2021178991, WO 2021136345, WO 2021190647, WO 2020219639, WO 2020182159, WO 2020155931, WO 2020038457, WO 2020219524, WO 2020173400, WO 2018204233, WO 2018204238, WO 2018169875, WO 2018117152, WO 2017215630, WO 2016070697, WO 2016027195, CN 117815195, CN 117815367, and CN 115969796, each of which is incorporated herein by reference in its entirety, including the compound structures disclosed therein which are specifically incorporated herein by reference.

In some embodiments, the JAK/STAT pathway inhibitor is a STAT inhibitor. In some embodiments, the STAT inhibitor is an inhibitor of STAT3 and/or STAT5. In some embodiments, the STAT inhibitor is a STAT3 degrader. In some embodiments, the STAT inhibitor is one or more of TTI-101, C-188-9, WP1066, VVD-130850, LLL12B, STA-21, SD-36, Stattic, S31-201, OPB-31121, and Napabucasin (BBI608). In some embodiments, reference to the term STAT inhibitor includes any such STAT inhibitor disclosed in any one of the following patent applications: WO 2024030628, WO 2023164680, WO 2023192960, WO 2023133336, WO2020206424, WO 2023107706, WO 2021150543, WO 2008151037, and CN 109288845, each of which is incorporated herein by reference in its entirety, including the compound structures disclosed therein which are specifically incorporated herein by reference.

### I) Epigenetic Modulators

Compositions and methods described herein may include a compound of the present invention in combination with one or more epigenetic modulators. Epigenetic modulators are a class of therapeutics that target enzymes responsible for modifying the structure and function of chromatin, the complex of DNA and proteins that make up chromosomes. These enzymes, including histone deacetylases (HDACs), histone methyltransferases (HMTs), and DNA methyltransferases (DNMTs), play critical roles in gene expression and regulation by modifying the packaging of DNA and affecting how it is read and transcribed. Epigenetic modulators work by altering the activity of these enzymes, either by inhibiting or enhancing their function, to regulate gene expression in specific ways. By targeting specific epigenetic modifications, such as acetylation, methylation, and DNA methylation, these therapies have the potential to treat a wide range of diseases, including cancer, inflammatory disorders, and neurological disorders.

### i) HDAC Inhibitors

In some embodiments, compositions and methods described herein may include a compound of the present invention in combination with one or more histone deacetylase (HDAC) inhibitors. A HDAC inhibitor may be administered or formulated in combination with a compound of the present invention and/or any additional therapeutic agent described herein. There are several classes of HDACs, including class I, class IIa, class IIb, class III, and class IV. Class I HDACs are further divided into HDAC1, HDAC2, HDAC3, and HDAC8, while class IIa HDACs include HDAC4, HDAC5, HDAC7, and HDAC9. Class Ilb HDACs consist of HDAC6 and HDAC10, and class III HDACs are known as sirtuins. HDAC inhibitors can target different classes of HDACs, and their specific effects on gene expression can vary depending on which HDACs they target. In some embodiments, a HDAC inhibitor is one or more of Vorinostat (Zolinza), Romidepsin (Istodax), Belinostat (Beleodaq), Panobinostat (Farydak), Entinostat (MS-275), Valproic acid (Depakene), Trichostatin A (TSA), Sodium butyrate, and Mocetinostat (MGCD0103). Non-limiting examples of HDAC inhibitors include trichostatin, sodium butyrate, apicidan, suberoyl anilide hydroamic acid, vorinostat, LBH 589, romidepsin, ACY-1215, and Panobinostat. In some embodiments, reference to the term HDAC inhibitor includes any such HDAC inhibitor disclosed in any one of the following patent applications: WO 2022110958, WO 2021252628, WO 2019204550, WO 2018178060, WO 2016126724, WO 2014143666, WO 2013041480, and WO 2006120456, each of which is incorporated herein by reference in its entirety, including the compound structures disclosed therein which are specifically incorporated herein by reference.

### ii) BET Inhibitors

In some embodiments, compositions and methods described herein may include a compound of the present invention in combination with one or more bromodomain and extra-terminal protein (BET) inhibitors. A BET inhibitor may be administered or formulated in combination with a compound of the present invention and/or any additional therapeutic agent described herein. BET (bromodomain and extra-terminal) proteins are a family of epigenetic reader proteins that recognize and bind to acetylated lysine residues on histones, leading to chromatin remodeling and gene expression regulation. There are four BET proteins in humans: BRD2, BRD3, BRD4, and BRDT. BET inhibitors specifically target the bromodomains of BET proteins, inhibiting their binding to acetylated lysine residues on histones and leading to alterations in gene expression. BET inhibitors are useful in the treatment of cancer and other diseases characterized by dysregulated gene expression. In some embodiments, a BET inhibitor is one or more of JQ1, I-BET762, OTX015, RVX-208, and CPI-0610. In some embodiments, reference to the term BET inhibitor includes any such BET inhibitor disclosed in any one of the following patent applications: WO 2022046682, WO 2022182857, WO 2021107657, WO 2021107656, WO 2020221006, WO 2020053660, WO 2018097977, WO 2017222977, WO 2017142881, WO 2015075665, WO 2015011084, and CN 113264930, each of which is incorporated herein by reference in its entirety, including the compound structures disclosed therein which are specifically incorporated herein by reference.

iii) EZH2 Inhibitors

In some embodiments, compositions and methods described herein may include a compound of the present invention in combination with one or more Enhancer of Zeste Homolog 2 (EZH2) inhibitors. An EZH2 inhibitor may be administered or formulated in combination with a compound of the present invention and/or any additional therapeutic agent described herein. EZH2 is a histone-lysine N-methyltransferase that is a member of the Polycomb repressive complex 2 (PRC2) family. EZH2 plays a crucial role in gene expression regulation, specifically by catalyzing the trimethylation of histone H3 at lysine 27 (H3K27me3), leading to transcriptional repression of target genes. EZH2 has been found to be overexpressed in several types of cancers and is associated with tumor progression and poor prognosis. In some embodiments, an EZH2 inhibitor is one or more of Tazemetostat, GSK2816126, and CPI-1205 (lirametostat). In some embodiments, reference to the term EZH2 inhibitor includes any such EZH2 inhibitor disclosed in any one of the following patent applications: WO 2023030299, WO 2022179584, WO 2020224607, WO 2021243060, WO 2021086069, WO 2019206155, WO 2018133795, WO 2018137639, WO 2017184999, WO 2017218953, WO 2016201328, WO 2015195848, WO 2013155317, WO 2013138361, and CN 114621191, each of which is incorporated herein by reference in its entirety, including the compound structures disclosed therein which are specifically incorporated herein by reference.

### iv) Co-REST Inhibitors

In some embodiments, compositions and methods described herein may include a compound of the present invention in combination with one or more Co-REST inhibitors. A Co-REST inhibitor may be administered or formulated in combination with a compound of the present invention and/or any additional therapeutic agent described herein. Co-REST is a transcriptional co-repressor protein that interacts with a variety of transcription factors to regulate gene expression. Co-REST acts by recruiting histone deacetylases (HDACs) to chromatin, leading to the repression of gene expression. Inhibition of Co-REST has been proposed as a potential therapeutic strategy for the treatment of various diseases, including neurodegenerative disorders and cancer. In some embodiments, a co-REST inhibitor is one or more of Nocodazole, NSC 1892, and Anacardic acid.

### v) EP300

In some embodiments, compositions and methods described herein may include a compound of the present invention in combination with one or more E1A-binding protein p300 (EP300) inhibitors. An EP300 inhibitor may be administered or formulated in combination with a compound of the present invention and/or any additional therapeutic agent described herein. EP300 is a transcriptional co-activator involved in the regulation of numerous cellular processes, including chromatin remodeling, DNA damage response, and cell cycle progression. EP300 acts as a histone acetyltransferase, catalyzing the transfer of acetyl groups to lysine residues on histone proteins, which leads to changes in chromatin structure and gene expression. EP300 activity has been implicated in diseases, such as cancer, cardiovascular and neurological disorders. In some embodiments, an EP300 inhibitor is one or more of C₆₄₆, A-485, NU9056, and L002. In some embodiments, reference to the term EP300 inhibitor includes any such EP300 inhibitor disclosed in any one of the following patent applications: WO 2021213521 and WO 2016044694, each of which is incorporated herein by reference in its entirety, including the compound structures disclosed therein which are specifically incorporated herein by reference.

### vi) LSD1

In some embodiments, compositions and methods described herein may include a compound of the present invention in combination with one or more Lysine-specific demethylase 1 (LSD1) inhibitors. A LSD1 inhibitor may be administered or formulated in combination with a compound of the present invention and/or any additional therapeutic agent described herein. LSD1 is an enzyme that plays a crucial role in regulating gene expression through histone modification. It specifically removes the methyl group from lysine 4 on histone 3, leading to gene repression. Dysregulation of LSD1 has been associated with various diseases including cancer and neurodegenerative disorders. In some embodiments, a LSD1 inhibitor is one or more of GSK2879552, IMG-7289, ORY-1001, IMG-8419, SP-2577, CC-90011, HCl-2509, and INCB059872. In some embodiments, reference to the term LSD1 inhibitor includes any such LSD1 inhibitor disclosed in any one of the following patent applications: WO 2021095840, WO 2021175079, WO 2021058024, WO 2020047198, WO 2020052649, WO 2020015745, WO 2020052647, WO 2018137644, WO 2017184934, WO 2017027678, WO 2017116558, WO 2017149463, WO 2016161282, WO 2015123465, WO 2015123424, WO 2013057322, WO 2013057320, WO 2012135113, CN 114805261, CN 111072610 CN 107174584, CN 110478352, CN 106432248, and CN 106045881, each of which is incorporated herein by reference in its entirety, including the compound structures disclosed therein which are specifically incorporated herein by reference.

vii) PRMT5

In some embodiments, compositions and methods described herein may include a compound of the present invention in combination with one or more Protein arginine methyltransferase 5 (PRMT5) inhibitors. A PRMT5 inhibitor may be administered or formulated in combination with a compound of the present invention and/or any additional therapeutic agent described herein. PRMT5 is a member of the PRMT family, which catalyzes the transfer of a methyl group from S-adenosylmethionine (SAM) to the nitrogen atoms of arginine residues in target proteins. PRMT5 is involved in various biological processes, including gene expression regulation, signal transduction, and DNA repair. In some embodiments, a PRMT5 inhibitor is one or more of TNG908, TNG462, AMG193, GSK591, EPZ015666, TC-E 5003, and MS023. In some embodiments, reference to the term PRMT5 inhibitor includes any such PRMT5 inhibitor disclosed in any one of the following patent applications: WO 2023001133, WO 2022206964, WO 2022153161, WO 2021068953, WO 2021088992, WO 2020259478, WO 2020205660, WO 2020250123, WO 2020033288, WO 2019102494, WO 2019112719, WO 2019180631, WO 2018065365, WO 2017153186, WO 2017212385, WO 2017032840, WO 2016022605, WO2014100695, WO 2014145214, WO 2014100719, CN 111825656, CN 114558014, CN 11304554, and CN 112778275, each of which is incorporated herein by reference in its entirety, including the compound structures disclosed therein which are specifically incorporated herein by reference.

viii) MAT2A

In some embodiments, compositions and methods described herein may include a compound of the present invention in combination with one or more methionine adenosyltransferase 2A (MAT2A) inhibitors. A MAT2A inhibitor may be administered or formulated in combination with a compound of the present invention and/or any additional therapeutic agent described herein. MAT2A is an enzyme that catalyzes the production of S-adenosylmethionine (SAM), which is an important cofactor in many biological processes, including DNA methylation, protein methylation, and polyamine synthesis. Elevated MAT2A expression has been associated with various cancers. In some embodiments, a MAT2A inhibitor is one or more of cycloleucine and 2-hydroxy-4-methylthiobutanoic acid. In some embodiments, reference to the term MAT2A inhibitor includes any such MAT2A inhibitor disclosed in any one of the following patent applications: WO 2022256808, WO 2022256806, WO 2019191470, and CN 115716831, each of which is incorporated herein by reference in its entirety, including the compound structures disclosed therein which are specifically incorporated herein by reference.

### ix) DOT1L

In some embodiments, compositions and methods described herein may include a compound of the present invention in combination with one or more Disruptor of Telomeric silencing 1-like (DOT1L) inhibitors. A DOT1L inhibitor may be administered or formulated in combination with a compound of the present invention and/or any additional therapeutic agent described herein. DOT1L is a histone methyltransferase enzyme that catalyzes the methylation of lysine 79 on histone H3. This modification is associated with transcriptional elongation and is important for the maintenance of gene expression programs. The DOT1L family includes enzymes that are involved in epigenetic regulation and transcriptional control, and their dysregulation has been linked to various diseases, including cancer. In some embodiments, a DOT1L inhibitor is one or more of EPZ-5676 (pinometostat) and EPZ-004777. In some embodiments, reference to the term DOT1L inhibitor includes any such DOT1L inhibitor disclosed in any one of the following patent applications: WO 2016090271, WO 2014100662, and CN 108997480, each of which is incorporated herein by reference in its entirety, including the compound structures disclosed therein which are specifically incorporated herein by reference.

iix) UBA1

In some embodiments, compositions and methods described herein may include a compound of the present invention in combination with one or more ubiquitin-activating enzyme inhibitors (e.g., a UBA1 inhibitor). A UBA1 inhibitor may be administered or formulated in combination with a compound of the present invention and/or any additional therapeutic agent described herein. UBA1, also known as ubiquitin-activating enzyme 1, is a key enzyme involved in the ubiquitination process, a fundamental cellular mechanism for protein degradation and regulation. Ubiquitination involves the covalent attachment of ubiquitin molecules to target proteins, marking them for degradation by the proteasome or modulating their activity, localization, or interactions within the cell. Several inhibitors have been developed to modulate UBA1 activity, with the aim of disrupting ubiquitination-mediated processes in diseased cells. These inhibitors include but are not limited to adenosine-based inhibitors which typically compete with ATP for binding to the active site of UBA1, thereby preventing the activation of ubiquitin (e.g., PYR-41 and MLN7243); covalent inhibitors which form irreversible bonds with specific amino acid residues in the active site of UBA1, leading to inhibition of its activity (e.g., TAK-243 (formerly known as MLN4924)); allosteric inhibitors which bind to sites on UBA1 distinct from the active site, inducing conformational changes that inhibit its catalytic activity (e.g., compound 2i); and fragment-based inhibitors which are designed based on smaller molecular fragments that bind to UBA1. In some embodiments, a UBA1 inhibitor is one or more of PYR-41, MLN7243, and TAK-243. In some embodiments, reference to the term UBA1 inhibitor includes any such UBA1 inhibitor disclosed in any one of the following patent applications: WO 2016069393 A1, WO 2016069392 A1, and JP 2013237627 A2, each of which is incorporated herein by reference in its entirety, including the compound structures disclosed therein which are specifically incorporated herein by reference.

### m) Additional Therapeutic Agents Useful for Combination Therapy

In some embodiments, compositions and methods described herein may include a compound of the present invention in combination with one or more Farnesyl transferase inhibitors. A farnesyl transferase inhibitor may be administered or formulated in combination with a compound of the present invention and/or any additional therapeutic agent described herein. Farnesyl transferase inhibitors (FTIs) are a class of drugs that target the farnesyl transferase enzyme, which plays a role in a process called protein prenylation. Protein prenylation is an important step in the process of activating certain proteins involved in signal transduction, cell growth, and differentiation. In some embodiments, a farnesyl transferase inhibitor is one or more of tipifarnib, lonafarnib, and rilapladib. In some embodiments, reference to the term farnesyl transferase inhibitor includes any such farnesyl transferase inhibitor disclosed in any one of the following patent applications: WO 2010057028, WO 2007042465, WO 200136395, WO 200064891, WO 200042849, WO 199938862, WO 199928315, WO 199829390, WO 199426723, CN 107312000, CN 107365310, KR 100375421, KR 100388790, each of which is incorporated herein by reference in its entirety, including the compound structures disclosed therein which are specifically incorporated herein by reference.

In some embodiments, compositions and methods described herein may include a compound of the present invention in combination with one or more casein kinase inhibitors. In some embodiments, a casein inhibitor is, SR-3029, a potent and ATP competitive CK10 and CK18 inhibitor.

In some embodiments, compositions and methods described herein may include one or more FLT3 inhibitors in combination with a compound of the present invention disclosed herein. FLT3 (Fms-like tyrosine kinase 3), also known as CD135, is a receptor tyrosine kinase (RTK) that plays a crucial role in regulating hematopoiesis, the process by which blood cells are formed. It is primarily expressed on hematopoietic stem cells (HSCs) and progenitor cells in the bone marrow, where it controls cell proliferation, survival, and differentiation. In some embodiments, a FLT3 inhibitor includes, but are not limited to, midostaurin, gilteritinib, sorafenib, quizartinib, crenolanib, ponatinib and quizartinib.

In some embodiments, compositions and methods described herein may include a compound of the present invention in combination with one or more one or more TGFb pathway inhibitors. In some embodiments, compositions and methods described herein may include one or more TGFb inhibitors. A TGFb inhibitor may be administered or formulated in combination with a compound of the present invention and/or any additional therapeutic agent described herein. TGFb (transforming growth factor beta) is a multifunctional cytokine involved in various cellular processes, including cell growth, differentiation, apoptosis, and immune response. Dysregulation of the TGFb signaling pathway has been implicated in various diseases, including cancer, fibrosis, and autoimmune disorders. In some embodiments, a TGFb inhibitor is one or more of galunisertib (LY2157299), and vactosertib (TEW-7197). In some embodiments, a TGFb inhibitor is one or more of Galunisertib, LY2157299, Fresolimumab, Lerdelimumab, Trabedersen, curcumin, resveratrol and small interfering RNA (siRNA) to silence TGFb receptor expression. In some embodiments, reference to the term TGFb inhibitor includes any such TGFb inhibitor disclosed in any one of the following patent applications: WO 2023043473, WO 2020104648, WO 2020128850, WO 2016140884, WO 2007018818, WO 2004024159, WO 200226935, WO 2002062753, WO 2002062776, and JP 2012087076, each of which is incorporated herein by reference in its entirety, including the compound structures disclosed therein which are specifically incorporated herein by reference.

In some embodiments, compositions and methods described herein may include a compound of the present invention in combination with one or more HSP90 inhibitors. A HSP90 inhibitor may be administered or formulated in combination with a compound of the present invention and/or any additional therapeutic agent described herein. HSP90, also known as heat shock protein 90, is a molecular chaperone that plays a critical role in regulating the folding, stability, and activity of a large number of client proteins involved in various cellular processes, including cell cycle progression, signal transduction, and apoptosis. In some embodiments, a HSP90 inhibitor is one or more of Geldanamycin and its derivatives (e.g., 17-AAG, 17-DMAG), KOS 953, Radicicol and its derivatives (e.g., PU—H71), SNX-2112, Ganetespib, AT13387, Onalespib, Luminespib, and KW-2478. In some embodiments, reference to the term HSP90 inhibitor includes any such HSP90 inhibitor disclosed in any one of the following patent applications: WO 2021137665, WO 2018200534, WO 2017151425, WO 2015200514, WO 2013053833, WO 2013009657, WO 2013119985, WO 2012138894, WO 2011044394, WO 2009097578, WO 2008115719, CN 105237533, and CN 104030904, each of which is incorporated herein by reference in its entirety, including the compound structures disclosed therein which are specifically incorporated herein by reference.

In some embodiments, compositions and methods described herein may include a compound of the present invention in combination with one or more Glutathione peroxidase 4 (GPX4) inhibitors. A GPX4 inhibitor may be administered or formulated in combination with a compound of the present invention and/or any additional therapeutic agent described herein. GPX4 is an antioxidant enzyme that plays a critical role in protecting cells against oxidative stress-induced cell death. GPX4 catalyzes the reduction of lipid hydroperoxides to their corresponding alcohols and acts as a regulator of ferroptosis, a form of regulated cell death driven by lipid peroxidation. In some embodiments, a GPX4 inhibitor is one or more of RSL3, ML162, DPI7, FINO2, MCB-613, CBS9106, ML210, ODSH, and TLN232. In some embodiments, reference to the term GPX4 inhibitor includes any such GPX4 inhibitor disclosed in any one of the following patent applications: WO 2021132592, US2021244715, and KR 20220115536, each of which is incorporated herein by reference in its entirety, including the compound structures disclosed therein which are specifically incorporated herein by reference.

In some embodiments, compositions and methods described herein may include a compound of the present invention in combination with one or more NRF2 inhibitors. A NRF2 inhibitor may be administered or formulated in combination with a compound of the present invention and/or any additional therapeutic agent described herein. NRF2 is a transcription factor that regulates the expression of genes involved in the cellular antioxidant response, detoxification, and other cytoprotective pathways. It plays a critical role in cellular defense mechanisms against oxidative stress and other forms of cellular damage. In some embodiments, a NRF2 inhibitor is one or more of ML385, Brusatol, CDDO-Im, RTA-408, and trigonelline. In some embodiments, reference to the term NRF2 inhibitor includes any such NRF2 inhibitor disclosed in any one of the following patent applications: WO 2023051088, WO 2021202720, KR 2022013610, and CN 107519168, each of which is incorporated herein by reference in its entirety, including the compound structures disclosed therein which are specifically incorporated herein by reference.

In some embodiments, compositions and methods described herein may include a compound of the present invention in combination with one or more TEA domain (TEAD) inhibitors. A TEAD inhibitor may be administered or formulated in combination with a compound of the present invention and/or any additional therapeutic agent described herein. TEAD is a family of transcription factors that play a key role in regulating gene expression during embryonic development and tissue homeostasis. The four members of the TEAD family (TEAD1-4) are transcriptional co-activators that bind to DNA through their conserved TEA domain and interact with other transcription factors to activate the expression of target genes. In some embodiments, a TEAD inhibitor is one or more of VT-107, a pan-TEAD, VT-104, Verteporfin, CA3, IAG933, K-975, IK-595, and Statins (see, e.g., Chapeau, Emilie and Schmelzle, Tobias (2023) IAG933, an oral selective YAP1-TAZ/pan-TEAD protein-protein interaction inhibitor (PPIi) with pre-clinical activity in monotherapy and combinations with MAPK inhibitors. Nature cancer). In some embodiments, reference to the term TEAD inhibitor includes any such TEAD inhibitor disclosed in any one of the following patent applications: WO 2023280254, WO 2023031781, WO 2022258040, WO 2020070181 WO 2018185266, and WO 2017064277, each of which is incorporated herein by reference in its entirety, including the compound structures disclosed therein which are specifically incorporated herein by reference.

In some embodiments, compositions and methods described herein may include a compound of the present invention in combination with one or more NOTCH/Gamma secretase inhibitors. A NOTCH/Gamma secretase inhibitor may be administered or formulated in combination with a compound of the present invention and/or any additional therapeutic agent described herein. In some embodiments, a NOTCH/Gamma secretase inhibitor is nirogacestat. In some embodiments, reference to the term NOTCH/Gamma secretase inhibitor includes any such NOTCH/Gamma secretase inhibitor disclosed in any one of the following patent applications: WO 2020208572, WO 2017200969, WO 2014047390, WO 2014047372, WO 2011041336, WO 2010090954, WO 2009008980, WO 2009087130, WO 2007110335, CN 103664904, CN 105560244, and KR 20200077480, each of which is incorporated herein by reference in its entirety, including the compound structures disclosed therein which are specifically incorporated herein by reference.

In some embodiments, compositions and methods described herein may include a compound of the present invention in combination with one or more Hedgehog inhibitors. A hedgehog inhibitor may be administered or formulated in combination with a compound of the present invention and/or any additional therapeutic agent described herein. The hedgehog (Hh) family of proteins are secreted signaling molecules that play a crucial role in embryonic development and tissue homeostasis in adults. The Hh signaling pathway is involved in regulating cell growth, differentiation, and survival. In some embodiments, a hedgehog inhibitor is one or more of Vismodegib (Erivedge), Sonidegib (Odomzo), and Glasdegib (Daurismo). In some embodiments, reference to the term hedgehog inhibitor includes any such hedgehog inhibitor disclosed in any one of the following patent applications: WO 2011063309, and CN 107163028, each of which is incorporated herein by reference in its entirety, including the compound structures disclosed therein which are specifically incorporated herein by reference.

Compositions and methods described herein may include a compound of the present invention in combination with one or more NFkB pathway inhibitors. An NFkB inhibitor may be administered or formulated in combination with a compound of the present invention and/or any additional therapeutic agent described herein. NF-kappa B (NFkB) is a family of transcription factors involved in regulating various cellular processes, including inflammation, immunity, cell survival, and proliferation. Non-limiting examples of NFkB inhibitors include Bortezomib (Velcade), Curcumin, Parthenolide, IKK inhibitors (e.g., IKK-16, BAY 11-7082), Resveratrol, Andrographolide and Proteasome inhibitors (e.g., MG132, lactacystin).

In some embodiments, the additional therapy is the administration of side-effect limiting agents (e.g., agents intended to lessen the occurrence or severity of side effects of treatment. For example, in some embodiments, the a compound of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor).

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, a compound of the present invention may be used as an adjuvant therapy after surgery. In some embodiments, a compound of the present invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, a compound of the present invention can render abnormal cells more sensitive to treatment with radiation for purposes of killing or inhibiting the growth of such cells. Accordingly, this disclosure further relates to a method for sensitizing abnormal cells in a mammal to treatment with radiation which comprises administering to the mammal an amount of a compound of the present disclosure, which amount is effective to sensitize abnormal cells to treatment with radiation. The amount of the compound in this method can be determined according to the means for ascertaining effective amounts of such compounds described herein. In some embodiments, a compound of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

In some embodiments, compositions and methods described herein may include a compound of the present invention in combination with one or more Claudin-18 targeting agents. A Claudin-18 targeting agents may be administered or formulated in combination with a compound of the present invention and/or any additional therapeutic agent described herein. Claudin-18 (e.g., claudin 18.2; CLDN18.2) has become a promising target for the treatment of patients with digestive malignancies, such as gastric cancer (GC), gastroesophageal junction (GEJ) cancer, esophageal cancer, and pancreatic cancer, because of its limited expression in healthy tissues and abnormal overexpression in a range of malignancies. Multiple clinical trials of CLDN18.2-targeted therapies, including monoclonal antibodies, bispecific antibodies, antibody-drug conjugates (ADCs), and chimeric antigen receptor (CAR) T-cell therapies, are ongoing, with some showing promising early results. Malignant transformation of gastric epithelial tissue leads to disruption of cell polarity and then to exposure of CLDN18.2 epitopes on the cell surface. Although targeted monoclonal antibodies are largely unable to access CLDN18.2 located in tight-junction supramolecular complexes in normal tissue, the perturbations in cell polarity that expose CLDN18.2 epitopes may theoretically enable CLDN18.2 targeted agents to bind to CLDN18.2 in malignant tissues with minimal off-target effects, making CLDN18.2 an attractive target for therapy. In some embodiments, a Claudin-18 targeting agent is one or more of Zolbetuximab, ASKB589, Osemitamab (TST001), PT886 (a bispecific antibody that targets CLDN18.2 and CD47), TJ-CD4B, CMG901 (an ADC that is composed of an antiCLDN18.2 monoclonal antibody joined to a cytotoxic payload, monomethyl auristatin E), and CT041 (autologous T cells genetically engineered to express a CLDN18.2-targeted CAR). In some embodiments, reference to the term Claudin-18 targeting agent includes any such Claudin-18 targeting agent disclosed in any one of the following patent applications: WO 2024081544, WO 2024131683, WO 2024137619, WO 2024140670, WO 2024136594, WO 2023034922, WO 2023046202, WO 2022203090, WO 2022133169, WO 2022100613, WO 2022256449, WO 2022136642, WO 2021155380, WO 2021129765, WO 2021011885, WO 2021058000, WO 2021218874, WO 2021027850, WO 2020156554, WO 2020025792, WO 2020114480, WO 2020211792, WO 2020239005, WO 2019219089, WO 2018157147, WO 2018108106, WO 2016166122, WO 2014146778, CN 118290582, CN 118203658, and CN 118286201, each of which is incorporated herein by reference in its entirety, including the compound structures disclosed therein which are specifically incorporated herein by reference.

In some embodiments, a therapeutic agent for combination therapy may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

An additional therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

An additional therapeutic agent may be an immune modulatory agent. For example, an additional therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, which interacts with a checkpoint protein. In some embodiments, the checkpoint inhibitor is an agent, such as an antibody, which interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/lg fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002. Non-limiting examples of immune modulatory agent includes targets identified in Table 4.

TABLE 4
Exemplary Immune Modulatory Targets

| Target | Biological Function |
|---|---|
| CTLA-4 | Inhibitory Receptor |
| PD-1 | Inhibitory Receptor |
| PD-L1 | Ligand for PD-1 |
| LAG-3 | Inhibitory Receptor |
| B7.1 | Costimulatory Molecule |
| B7-H3 | Inhibitory Ligand |
| B7-H4 | Inhibitory Ligand |
| TIM3 | Inhibitory Receptor |
| VISTA | Inhibitory Receptor |
| CD137 | Costimulatory Molecule |
| OX-40 | Costimulatory Receptor |
| CD40 agonist | Costimulatory Molecule |
| CD40 agonist + FLT3 ligand | Costimulatory Molecule |
| CD27 | Costimulatory Receptor |
| CCR4 | Costimulatory Receptor |
| GITR | Costimulatory Receptor |
| NKG2D | Activating Receptor |
| KIR | Costimulatory Receptor |
| NKG2A | Inhibitory Receptor |
| ENPP1 | Inhibitory Receptor |
| TIGIT | Inhibitory Receptor |
| A2aR | Inhibitory Receptor |
| CD73 | Inhibitory Receptor |
| CD39 | Inhibitory Receptor |
| PVRIG | Inhibitory Receptor |
| IDO | Inhibitory enzyme |
| CSF1R | Inhibitory Receptor |
| LIF | Inhibitory Cytokine |
| CD47 | Inhibitory Receptor |
| SIRPa | Inhibitory Receptor |
| IL-2 | Effector Cytokines |
| IL-15 | Effector Cytokines |
| IL-12 | Effector Cytokines |
| TREM2 | Receptor |
| TGFb | Multifunctional Cytokine |
| CD73/TGFb trap | Multifunctional Cytokine |
| TCR-T cells directed to KRAS^{MUT}, | Cell therapy |
| mesothelin, or PRAME | |
| mRNA cancer vaccines | vaccines |
| BiTEs | Bi-specific T-cell engager |
| Dual EP2/EP4 inhibitor | E-prostanoid receptor |
| Gamma delta T Cells | Cell therapy |
| NK cells | Cell therapy |

CTLA4, cytotoxic T-lymphocyte-associated antigen 4;
LAG3, lymphocyte activation gene 3;
PD-1, programmed cell death protein 1;
PD-L1, PD-1 ligand;
TIM3, T cell membrane protein 3;
VISTA, V-domain immunoglobulin (Ig)-containing suppressor of T-cell activation;
KIR, killer IgG-like receptor, APC (Antigen Presenting Cells);
TREM2 (Triggering receptor expressed on myeloid cells 2);
TGF-b (Transforming growth factor beta)

An additional therapeutic agent may be an anti-TIGIT antibody, such as MBSA43, BMS-986207, MK-7684, COM902, AB154, MTIG7192A or OMP-313M32 (etigilimab).

In some embodiments, the combination therapy includes a compound of the present invention and a cancer vaccine composition. In some embodiments, the cancer vaccine composition is HB-700, mRNA-4157, mRNA-5671, BNT111, GVAX Pancreas, IMA901, DCVax, SOT101, Sipuleucel-T, PROSTVAC-VF or TG01.

An additional therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irinotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18: 233a (1999), and Douillard et al., Lancet 355 (9209): 1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4 (5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux@), rituximab (Rituxan@), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelaamine and thiotepa), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as luteinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), cell surface monoclonal antibodies (e.g., anti-CD38 (HUMAX-CD38), anti-CSI (e.g., elotuzumab, P13K/Akt inhibitors (e.g., perifosine), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), and cFMS inhibitors (e.g., ARRY-382).

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing. In some embodiments, the anti-cancer agent is JAB-3312.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune modulatory therapies, such as an immune checkpoint inhibitor. In some embodiments, a therapeutic agent may be a pan-RTK inhibitor, such as afatinib.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a RAS (ON) inhibitor of the present disclosure is used in combination with a MEK inhibitor and a SOS1 inhibitor. In some embodiments, a RAS (ON) inhibitor of the present disclosure is used in combination with a PD-L1 inhibitor and a SOS1 inhibitor. In some embodiments, a RAS (ON) inhibitor of the present disclosure is used in combination with a PD-L1 inhibitor and a SHP2 inhibitor. In some embodiments, a RAS (ON) inhibitor of the present disclosure is used in combination with a MEK inhibitor and a SHP2 inhibitor. In some embodiments, the cancer is colorectal cancer, and the treatment comprises administration of a Ras inhibitor of the present disclosure in combination with a second or third therapeutic agent.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110 (1): 186-192; Thompson et al., Clin. Cancer Res. 2007, 13 (6): 1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with a compound of the present invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloproteinase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), EGFR inhibitory agents (e.g., antibodies or antigen binding regions that specifically bind thereto) such as Vectibix® (panitumumab), erlotinib (Tarceva@), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXIGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with a compound of the present invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with a compound of the present invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-la, interferon beta-lb, interferon gamma, natural interferon gamma-la, interferon gamma-lb, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with a compound of the present invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a QDa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHZ, a Bruker Ascend 500 MHz instrument, or a Varian 400 MHz, and the raw data was analyzed with either TopSpin or Mestrelab Mnova.

### Synthesis of Intermediates

### Intermediate A. Synthesis of 3-(5-bromo-1-ethyl-2-[2-[(1S)-1-methoxyethyl]pyridin-3-yl]indol-3-yl)-2,2-dimethylpropan-1-ol

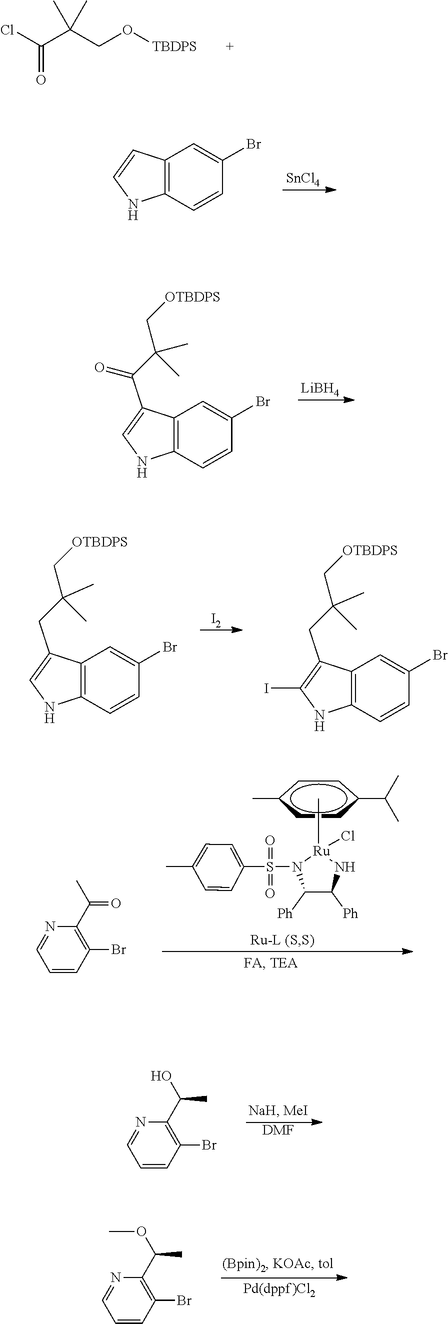

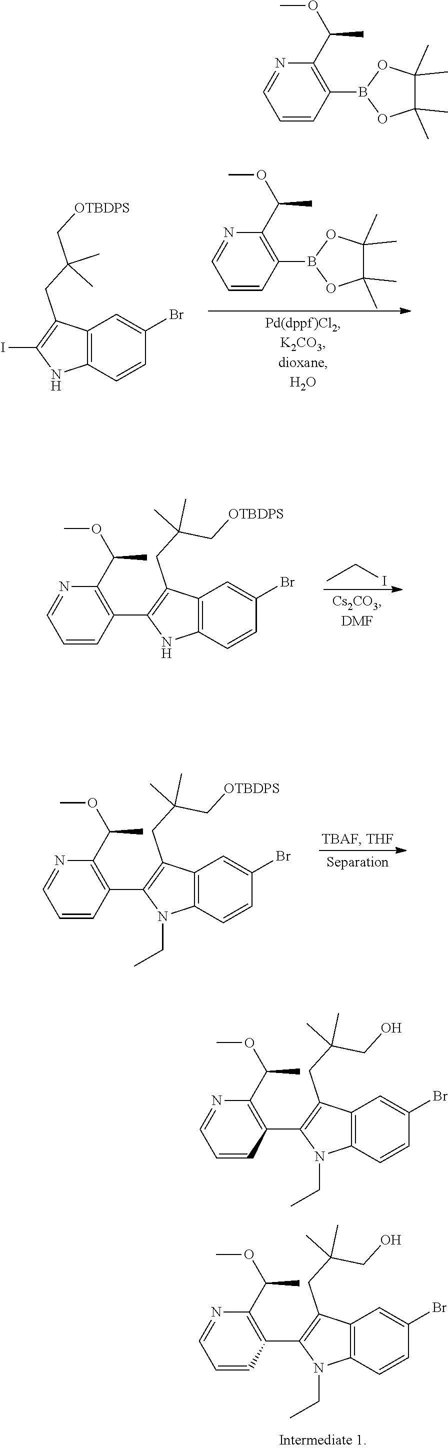

### Step 1

To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl4 in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (400 mL), dried over Na₂SO₄ and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI): m/z [M+Na]calc'd for C₂₉H₃₂BrNO₂SiNa 556.1; found 556.3.

### Step 2

To a mixture of 1-(5-bromo-1H-indol-3-yl)-3-((tert-butyldiphenylsilyl)oxy)-2,2-dimethylpropan-1-one (50 g, 93.6 mmol) in THF (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered and the filtrate concentrated under reduced pressure. The residue was diluted with DCM (50 mL), cooled to 10° C. and diludine (9.5 g, 37.4 mmol) and TsOH. H₂O (890 mg, 4.7 mmol) added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI): m/z [M+H] calc'd for C₂₉H₃₄BrNOSi 519.2; found 520.1; 1H NMR (400 MHZ, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

### Step 3

To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THF (15 mL) at rt was added AgOTf (888 mg, 3.5 mmol). The mixture was stirred at rt for 2 h, then diluted with EtOAc (200 mL) and washed with saturated Na₂S₂O₃ (100 mL), dried over anhydrous Na₂SO₄ and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. 1H NMR (400 MHZ, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

### Step 4

To a stirred mixture of HCOOH (66.3 g, 1.44 mol) in TEA (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to rt and 1-(3-bromopyridin-2-yl) ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄ and filtered. The filtrate was concentrated under reduced pressure and the residue was purified by silica gel column chromatography to give (1S)-1-(3-bromopyridin-2-yl) ethanol (100 g, 74% yield) as an oil. LCMS (ESI): m/z [M+H] calc'd for C₇H₈BrNO 201.1; found 201.9.

### Step 5

To a stirred mixture of (1S)-1-(3-bromopyridin-2-yl) ethanol (100 g, 495 mmol) in DMF (1 L) at 0° C. was added NaH, 60% dispersion in oil (14.25 g, 594 mmol) in portions. The mixture was stirred at 0° C. for 1 h. MeI (140.5 g, 990 mmol) was added dropwise at 0° C. and the mixture was allowed to warm to rt and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1S)-1-methoxyethyl]pyridine (90 g, 75% yield) as an oil. LCMS (ESI): m/z [M+H] calc'd for C₈H₁₀BrNO 215.0; found 215.9.

### Step 6

To a stirred mixture of 3-bromo-2-[(1S)-1-methoxyethyl]pyridine (90 g, 417 mmol) and Pd(dppf)Cl₂ (30.5 g, 41.7 mmol) in toluene (900 mL) at rt under an atmosphere of Ar was added bis(pinacolato)diboron (127 g, 500 mmol) and KOAc (81.8 g, 833 mmol) in portions. The mixture was heated to 100° C. and stirred for 3 h. The filtrate was concentrated under reduced pressure and the residue was purified by Al₂O₃ column chromatography to give 2-[(1S)-1-methoxyethyl]-3-(4,4,5,5-tetramethyl-1,3,2-dioxaborolan-2-yl)pyridine (100 g, 63% yield) as a semi-solid. LCMS (ESI): m/z [M+H] calc'd for C₁₄H₂₂BNO₃ 263.2; found 264.1.

### Step 7

To a stirred mixture of 5-bromo-3-[3-[(tert-butyldiphenylsilyl)oxy]-2,2-dimethylpropyl]-2-iodo-1H-indole (140 g, 217 mmol) and 2-[(1S)-1-methoxyethyl]-3-(4,4,5,5-tetramethyl-1,3,2-dioxaborolan-2-yl)pyridine (100 g, 380 mmol) in 1,4-dioxane (1.4 L) at rt under an atmosphere of Ar was added K₂CO₃ (74.8 g, 541 mmol), Pd(dppf)Cl₂ (15.9 g, 21.7 mmol) and H₂O (280 mL) in portions. The mixture was heated to 85° C. and stirred for 4 h, then cooled, H₂O (5 L) added and the mixture extracted with EtOAc (3×2 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 45% yield) as a solid. LCMS (ESI): m/z [M+H] calc'd for C₃₇H₄₃BrN₂O₂Si 654.2; found 655.1.

### Step 8

To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and Etl (33.8 g, 217 mmol) in portions. The mixture was warmed to rt and stirred for 16 h then H₂O (4 L) added and the mixture extracted with EtOAc (3×1.5 L). The combined organic layers were washed with brine (2×1 L), dried over anhydrous Na₂SO₄ and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI): m/z [M+H] calc'd for C₃₉H₄₇BrN₂O₂Si 682.3; found 683.3.

### Step 9

To a stirred mixture of TBAF (172.6 g, 660 mmol) in THF (660 mL) at rt under an atmosphere of N₂ was added 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 97 mmol) in portions. The mixture was heated to 50° C. and stirred for 16 h, cooled, diluted with H₂O (5 L) and extracted with EtOAc (3×1.5 L). The combined organic layers were washed with brine (2×1 L), dried over anhydrous Na₂SO₄ and filtered. After filtration, the filtrate was concentrated under reduced pressure. The filtrate was concentrated under reduced pressure and the residue was purified by silica gel column chromatography to give two atropisomers of 3-(5-bromo-1-ethyl-2-(2-((S)-1-methoxyethyl)pyridin-3-yl)-1H-indol-3-yl)-2,2-dimethylpropan-1-ol (as single atropisomers) both as solids. (Combined 30 g, 62% yield) both as a solid. LCMS (ESI): m/z [M+H] calc'd for C₂₃H₂₉BrN₂O₂ 444.1; found 445.1.

### Intermediate A. Alternative Synthesis Through Fisher Indole Route

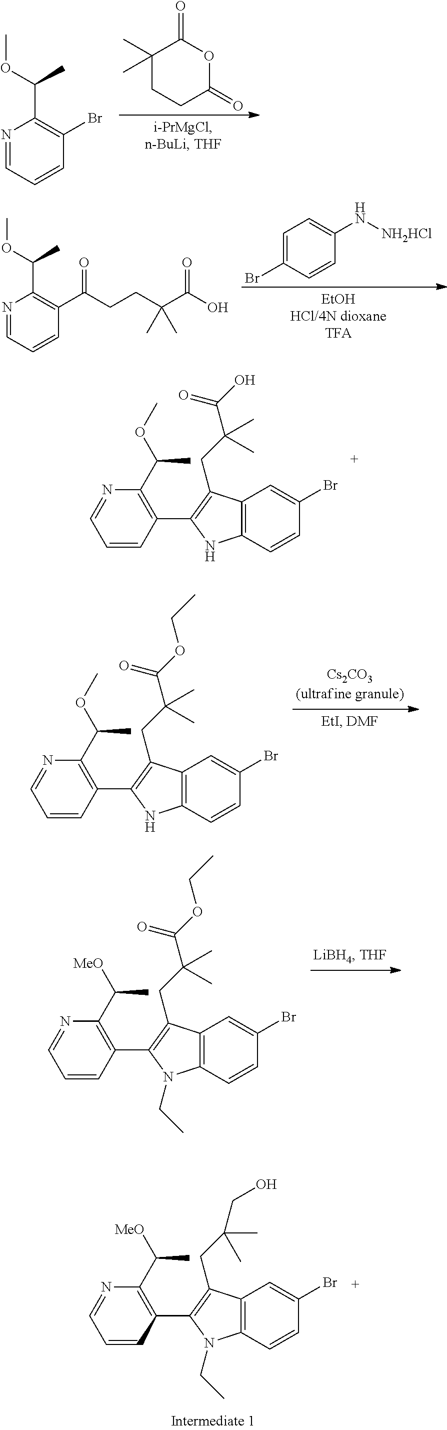

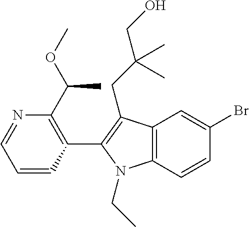

### Step 1

To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THF (0.5 L) added dropwise over 30 min at −10° C. The resulting mixture was warmed to −5° C. and stirred for 1 h, then 3,3-dimethyloxane-2,6-dione (118 g, 833 mmol) in THF (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with ice-water (3 L) and extracted with EtOAc (3×2.5 L). The combined organic layers were dried over anhydrous Na₂SO₄, filtered, the filtrate was concentrated under reduced pressure and the residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI): m/z [M+H] calc'd for C₁₅H₂₁NO₄ 279.2; found 280.1.

### Step 2

To a mixture of 5-[2-[(1S)-1-methoxyethyl]pyridin-3-yl]-2,2-dimethyl-5-oxopentanoic acid (78 g, 279 mmol) in EtOH (0.78 L) at rt under an atmosphere of N₂ was added (4-bromophenyl) hydrazine HCl salt (68.7 g, 307 mmol) in portions. The mixture was heated to 85° C. and stirred for 2 h, cooled to rt, then 4M HCl in 1,4-dioxane (69.8 mL, 279 mmol) added dropwise. The mixture was heated to 85° C. and stirred for an additional 3 h, then concentrated under reduced pressure and the residue was dissolved in TFA (0.78 L). The mixture was heated to 60° C. and stirred for 1.5, concentrated under reduced pressure and the residue adjusted to PH ˜5 with saturated NaHCO₃, then extracted with EtOAc (3×1.5 L). The combined organic layers were dried over anhydrous Na₂SO₄, filtered, the filtrate concentrated under reduced pressure and the residue was purified by silica gel column chromatography to give 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl)pyridin-3-yl)-1H-indol-3-yl)-2,2-dimethylpropanoate (78 g, crude). LCMS (ESI): m/z [M+H] calc'd for C₂₁H₂₃BrN₂O₃ 430.1 and C₂₃H₂₇BrN₂O₃ 458.1; found 431.1 and 459.1.

### Step 3

To a mixture of 3-(5-bromo-2-[2-[(1S)-1-methoxyethyl]pyridin-3-yl]-1H-indol-3-yl)-2,2-dimethylpropanoic acid and ethyl(S)-3-(5-bromo-2-(2-(1-methoxyethyl)pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. Etl (215 g, 1.38 mmol) in DMF (200 mL) was then added dropwise at 0° C. The mixture was warmed to rt and stirred for 4 h then diluted with brine (5 L) and extracted with EtOAc (3×2.5 L). The combined organic layers were washed with brine (2×1.5 L), dried over anhydrous Na₂SO₄ and filtered. The filtrate was concentrated under reduced pressure and the residue was purified by silica gel column chromatography to give ethyl 3-(5-bromo-1-ethyl-2-[2-[(1S)-1-methoxyethyl]pyridin-3-yl]indol-3-yl)-2,2-dimethylpropanoate (160 g, 57% yield) as a solid. LCMS (ESI): m/z [M+H] calc'd for C₂₅H₃₁BrN₂O₃ 486.2; found 487.2.

### Step 4

To a mixture of ethyl 3-(5-bromo-1-ethyl-2-[2-[(1S)-1-methoxyethyl]pyridin-3-yl]indol-3-yl)-2,2-dimethylpropanoate (160 g, 328 mmol) in THF (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄ and filtered. The filtrate was concentrated under reduced pressure and the residue was purified by silica gel column chromatography to give to two atropisomers of 3-(5-bromo-1-ethyl-2-(2-((S)-1-methoxyethyl)pyridin-3-yl)-1H-indol-3-yl)-2,2-dimethylpropan-1-ol (as single atropisomers) (60 g, 38% yield) and (40 g, 26% yield) both as solids. LCMS (ESI): m/z [M+H] calc'd for C₂₃H₂₉BrN₂O₂ 444.1; found 445.2.

### Intermediate B. Synthesis of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate

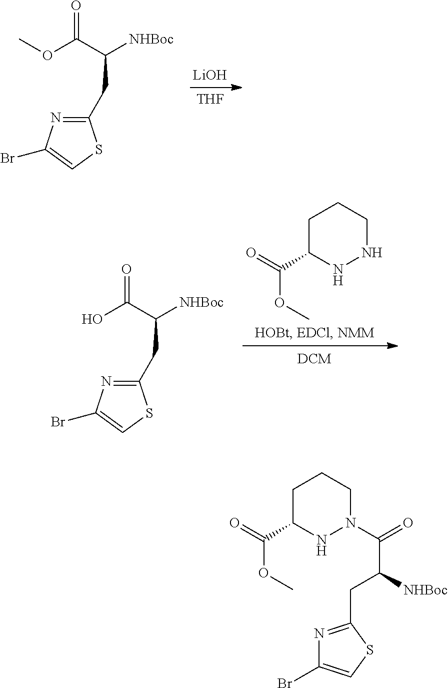

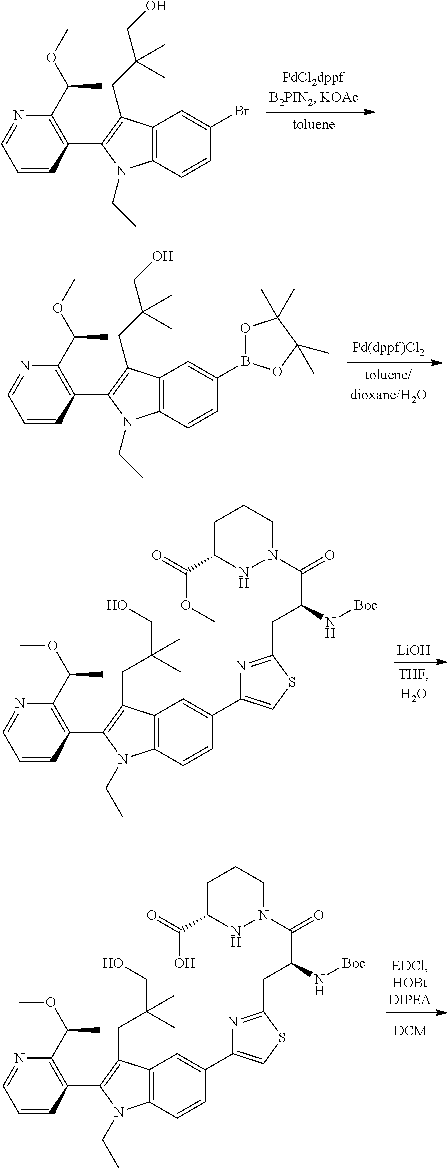

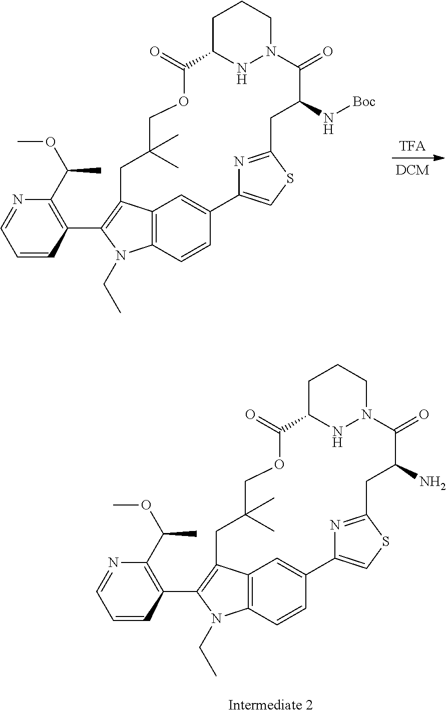

### Step 1

To a solution of methyl (2S)-3-(4-bromo-1,3-thiazol-2-yl)-2-[(tert-butoxycarbonyl)amino]propanoate (110 g, 301.2 mmol) in THF (500 mL) and H₂O (200 mL) at room temperature was added LiOH (21.64 g, 903.6 mmol). The solution was stirred for 1 h and was then concentrated under reduced pressure. The residue was adjusted to pH 6 with 1 M HCl and then extracted with DCM (3×500 mL). The combined organic layers were, dried over Na₂SO₄, filtered, and concentrated under reduced pressure to give(S)-3-(4-bromothiazol-2-yl)-2-((tert-butoxycarbonyl)amino) propanoic acid (108 g, crude). LCMS (ESI): m/z [M+H] calc'd for C₁₁H₁₆BrN₂O₄S 351.0; found 351.0.

### Step 2

To a solution of(S)-3-(4-bromothiazol-2-yl)-2-((tert-butoxycarbonyl)amino) propanoic acid (70 g, 199.3 mmol) in DCM (500 mL) at 0° C. was added methyl (3S)-1,2-diazinane-3-carboxylate bis(trifluoroacetic acid) salt (111.28 g, 298.96 mmol), NMM (219.12 mL. 1993.0 mmol), EDCl (76.41 g, 398.6 mmol) and HOBt (5.39 g, 39.89 mmol). The solution was warmed to room temperature and stirred for 1 h. The reaction was then quenched with H₂O (500 mL) and was extracted with EtOAc (3×500 mL). The combined organic layers were dried over Na₂SO₄, filtered, and concentrated under reduced pressured. The residue was purified by silica gel column chromatography to give methyl(S)-1-((S)-3-(4-bromothiazol-2-yl)-2-((tert-butoxycarbonyl)amino) propanoyl) hexahydropyridazine-3-carboxylate (88.1 g, 93% yield). LCMS (ESI): m/z [M+H] calc'd for C₁₇H₂₆BrN₄O₅S 477.1; found 477.1.

### Step 3

To a solution of 3-(5-bromo-1-ethyl-2-(2-((S)-1-methoxyethyl)pyridin-3-yl)-1H-indol-3-yl)-2,2-dimethylpropan-1-ol (60 g, 134.7 mmol) in toluene (500 mL) at room temperature was added bis(pinacolato)diboron (51.31 g, 202.1 mmol), Pd(dppf)Cl₂ (9.86 g, 13.4 mmol), and KOAc (26.44 g, 269 mmol). The reaction mixture was then heated to 90° C. and stirred for 2 h. The reaction solution was then cooled to room temperature and concentrated under reduced pressure. The residue was purified by silica gel column chromatography to give(S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (60.6 g, 94% yield). LCMS (ESI): m/z [M+H] calc'd for C₂₉H₄₂BN₂O₄ 493.32; found 493.3.

### Step 4

To a solution of(S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl(S)-1-((S)-3-(4-bromothiazol-2-yl)-2-((tert-butoxycarbonyl)amino) propanoyl) hexahydropyridazine-3-carboxylate (43.62 g, 91.4 mmol), K₃PO₄ (32.23 g, 152.3 mmol) and Pd(dppf)Cl₂ (8.91 g, 12.18 mmol). The resulting solution was heated to 70° C. and stirred overnight. The reaction mixture was then cooled to room temperature and was quenched with H₂O (200 mL). The mixture was extracted with EtOAc and the combined organic layers were dried over Na₂SO₄, filtered, and concentrated under reduced pressure. The residue was purified by silica gel column chromatography to give methyl(S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl) thiazol-2-yl) propanoyl) hexahydropyridazine-3-carboxylate (39.7 g, 85% yield). LCMS (ESI): m/z [M+H] calc'd for C₄₀H₅₅N₆O₇S 763.4; found 763.3.

### Step 5

To a solution of methyl(S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl) thiazol-2-yl) propanoyl) hexahydropyridazine-3-carboxylate (39.7 g, 52.0 mmol) in THF (400 mL) and H₂O (100 mL) at room temperature was added LiOH·H₂O (3.74 g, 156.2 mmol). The mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to give(S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl) thiazol-2-yl) propanoyl) hexahydropyridazine-3-carboxylic acid (37.9 g, crude). LCMS (ESI): m/z [M+H] calc'd for C₃₉H₅₃N₆O₇S 749.4; found 749.4.

### Step 6

To a solution of(S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl) thiazol-2-yl) propanoyl) hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCl (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel column chromatography to give tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (30 g, 81% yield). LCMS (ESI): m/z [M+H] calc'd for C₃₉H₅₁N₆O₆S 731.4; found 731.3.

### Step 7

To a solution of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (6 g, 8.21 mmol) in DCM (60 mL) at 0° C. was added TFA (30 mL). The mixture was stirred for 1 h and was then concentrated under reduced pressure to give (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-11H-8-oxa-2(4,2)-thiazola-1(5,3)-indola-6(1,3)-pyridazinacycloundecaphane-5,7-dione (7.0 g, crude). LCMS (ESI): m/z [M+H] calc'd for C₃₄H₄₂N₆₀₄S 631.3; found: 630.3.

### Intermediate C. Synthesis of tert-butyl ((6³S,4S,Z)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate

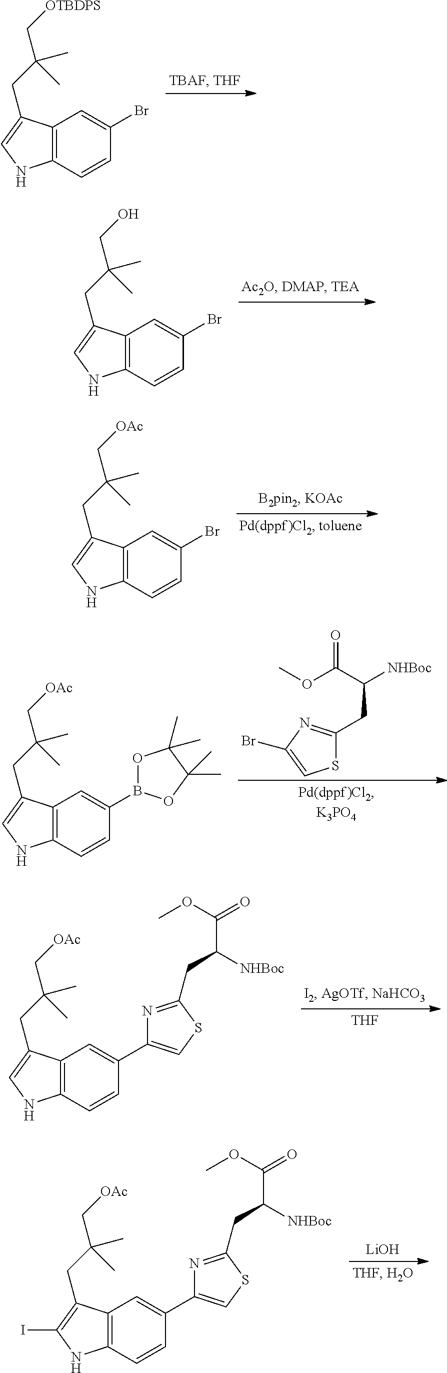

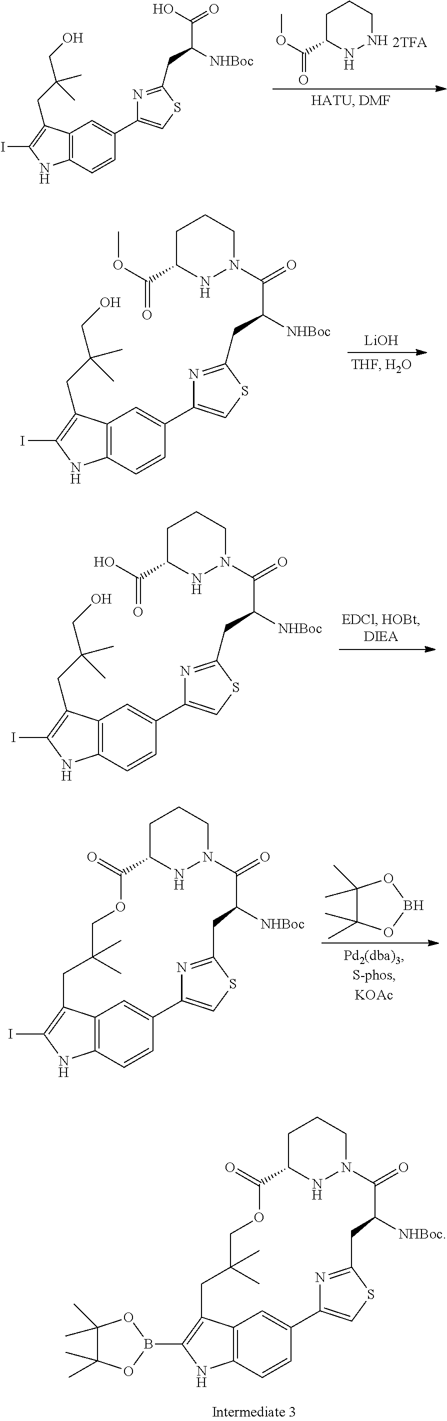

### Step 1

To a stirred solution of 5-bromo-3-[3-[(tert-butyldiphenylsilyl)oxy]-2,2-dimethylpropyl]-1H-indole (100 g, 192.0 mmol) in THF (1000 mL) were added TBAF (261.17 g, 998.8 mmol) in portion at room temperature. The resulting mixture was stirred for 16 h at 50° C. The resulting mixture was concentrated under reduced pressure. The resulting mixture was extracted with EtOAc (2 L). The combined organic layers were washed with water (6 L), dried over anhydrous Na₂SO₄. After filtration, the filtrate was concentrated under reduced pressure. The residue was purified by silica gel column chromatography, eluted with PE/EtOAc (3:1) to afford 3-(5-bromo-1H-indol-3-yl)-2,2-dimethylpropan-1-ol (54 g, 96.63%). LCMS (ESI): m/z [M+H] calc'd for C₁₃H₁₆BrNO 281.0; found 282.0.

### Step 2

To a stirred solution of 3-(5-bromo-1H-indol-3-yl)-2,2-dimethylpropan-1-ol (54 g, 191.3 mmol) in DCM (300 mL) were added TEA (58.09 g, 574.1 mmol) and Ac₂O (18.95 g, 185.6 mmol) and DMAP (1.17 g, 9.5 mmol) dropwise at 0° C. The resulting mixture was washed with water (3×500 mL), dried over anhydrous Na₂SO₄. After filtration, the filtrate was concentrated under reduced pressure. This resulted in 3-(5-bromo-1H-indol-3-yl)-2,2-dimethylpropyl acetate (54 g, 80.6%) as a yellow solid. The crude product was used in the next step directly without further purification. LCMS (ESI): m/z [M+H] calc'd for C₁₅H₁₈BrNO₂ 323.0; found 324.0.

### Step 3

To a stirred solution of 3-(5-bromo-1H-indol-3-yl)-2,2-dimethylpropyl acetate (54 g, 166.5 mmol) in toluene (600 mL) were added KOAc (40.87 g, 416.3 mmol) and B₂pin₂ (105.76 g, 416.3 mmol) and Pd(dppf)Cl₂ (12.19 g, 16.6 mmol) in portions at room temperature under argon atmosphere. The resulting mixture was stirred for 3 h at 90° C. under argon atmosphere. The resulting mixture was concentrated under reduced pressure. The resulting mixture was extracted with EtOAc (1 L). The combined organic layers were washed with water (3×1 L), dried over anhydrous Na₂SO₄. After filtration, the filtrate was concentrated under reduced pressure. The residue was purified by silica gel column chromatography, eluted with PE/EtOAc (3:1) to afford 2,2-dimethyl-3-[5-(4,4,5,5-tetramethyl-1,3,2-dioxaborolan-2-yl)-1H-indol-3-yl]propyl acetate borane (55 g, 76.57%) as a yellow solid. LCMS (ESI): m/z [M+H] calc'd for C₂₁H₃₀BNO₄ 371.2; found 372.2.

### Step 4

To a stirred solution of 2,2-dimethyl-3-[5-(4,4,5,5-tetramethyl-1,3,2-dioxaborolan-2-yl)-1H-indol-3-yl]propyl acetate (54 g, 145.443 mmol) and methyl (2S)-3-(4-bromo-1,3-thiazol-2-yl)-2-[(tert-butoxycarbonyl)amino]propanoate (79.68 g, 218.1 mmol) and K₃PO₄ (77.18 g, 363.6 mmol) in toluene (330 mL) and dioxane (110 mL) and H₂O (110 mL) were added Pd(dppf)Cl₂ (10.64 g, 14.5 mmol) in portions at room temperature under argon atmosphere. The resulting mixture was stirred for 36 h at 70° C. under argon atmosphere. The resulting mixture was concentrated under vacuum. The resulting mixture was extracted with EtOAc (3 L). The combined organic layers were washed with water (3×2 L), dried over anhydrous Na₂SO₄. After filtration, the filtrate was concentrated under reduced pressure. The residue was purified by silica gel column chromatography, eluted with PE/EtOAc (1:1) to afford methyl (2S)-3-(4-[3-[3-(acetyloxy)-2,2-dimethylpropyl]-1H-indol-5-yl]-1,3-thiazol-2-yl)-2-[(tert-butoxycarbonyl)amino]propanoate (54 g, 60.78%) as a yellow oil. LCMS (ESI): m/z [M+H] calc'd for C₂₇H₃₅N₃O₆S 529.2; found 530.2.

### Step 5

To a stirred solution of methyl (2S)-3-(4-[3-[3-(acetyloxy)-2,2-dimethylpropyl]-1H-indol-5-yl]-1,3-thiazol-2-yl)-2-[(tert-butoxycarbonyl)amino]propanoate (54 g, 101.954 mmol) in THF (450 mL) were added NaHCO₃ (10.28 g, 122.3 mmol) and AgOTf (31.44 g, 122.3 mmol) dropwise at 0° C. To the stirred solution was added 12 (23.29 g, 91.6 mmol) in THF (100 mL) dropwise at 0° C. The resulting mixture was stirred for 15 min at 0° C. The reaction was quenched with sat. Na₂S₂O₃ (aq.) at 0° C. The resulting mixture was extracted with EtOAc (1 L). The combined organic layers were washed with water (3 L), dried over anhydrous Na₂SO₄. After filtration, the filtrate was concentrated under reduced pressure. The residue was purified by silica gel column chromatography, eluted with PE/EtOAc (5:1) to afford methyl (2S)-3-(4-[3-[3-(acetyloxy)-2,2-dimethylpropyl]-2-iodo-1H-indol-5-yl]-1,3-thiazol-2-yl)-2-[(tert-butoxycarbonyl)mino]propanoate (40 g, 53.80%) as a yellow solid. LCMS (ESI): m/z [M+H] calc'd for C₂₇H₃₄IN₃O₆S 655.1; found 656.1.

### Step 6

To a stirred solution of methyl (2S)-3-(4-[3-[3-(acetyloxy)-2,2-dimethylpropyl]-2-iodo-1H-indol-5-yl]-1,3-thiazol-2-yl)-2-[(tert-butoxycarbonyl)amino]propanoate (40 g, 61.01 mmol) in THF (300 mL) and H₂O (100 mL) were added LiOH (4.38 g, 183.05 mmol) dropwise at 0° C. The resulting mixture was stirred for overnight at room temperature. The residue was acidified to pH 6 with conc. HCl. The resulting mixture was extracted with EtOAc (500 mL). The combined organic layers were washed with water (3×500 mL), dried over anhydrous Na₂SO₄. After filtration, the filtrate was concentrated under reduced pressure. This resulted in (2S)-2-[(tert-butoxycarbonyl)amino]-3-[4-[3-(3-hydroxy-2,2-dimethylpropyl)-2-iodo-1H-indol-5-yl]-1,3-thiazol-2-yl]propanoic acid (40 g, crude) as a yellow oil. The crude product was used in the next step directly without further purification. LCMS (ESI): m/z [M+H] calc'd for C₂₄H₃₀IN₃O₅S 599.1.1; found 600.1.

### Step 7

To a stirred solution of (2S)-2-[(tert-butoxycarbonyl)amino]-3-[4-[3-(3-hydroxy-2,2-dimethylpropyl)-2-iodo-1H-indol-5-yl]-1,3-thiazol-2-yl]propanoic acid (40 g, 66.72 mmol) and methyl (3S)-1,2-diazinane-3-carboxylate (28.86 g, 200.17 mmol) and HOBT (1.8 g, 13.35 mmol) and DIEA (172.47 g, 1334.5 mmol) in DCM (350 mL) were added EDCl (31.98 g, 166.8 mmol) dropwise at 0° C. The resulting mixture was stirred for overnight at room temperature under nitrogen atmosphere. The resulting mixture was washed with water (1.5 L), dried over anhydrous Na₂SO₄. After filtration, the filtrate was concentrated under reduced pressure. The residue was purified by silica gel column chromatography, eluted with PE/EtOAc (1:1) to afford methyl (3S)-1-[(2S)-2-[(tert-butoxycarbonyl)amino]-3-[4-[3-(3-hydroxy-2,2-dimethylpropyl)-2-iodo-1H-indol-5-yl]-1,3-thiazol-2-yl]propanoyl]-1,2-diazinane-3-carboxylate (28 g, 43.9%) as a yellow oil. LCMS (ESI): m/z [M+H] calc'd for C₃₀H₄₀IN₅O₆S 725.1.1; found 726.1.

### Step 8

To a stirred solution of methyl (3S)-1-[(2S)-2-[(tert-butoxycarbonyl)amino]-3-[4-[3-(3-hydroxy-2,2-dimethylpropyl)-2-iodo-1H-indol-5-yl]-1,3-thiazol-2-yl]propanoyl]-1,2-diazinane-3-carboxylate (28 g, 38.5 mmol) in THF (240 mL) were added LiOH (2.77 g, 115.7 mmol) in H₂O (80 mL) dropwise at 0° C. The resulting mixture was stirred for 2 h at room temperature. The mixture was acidified to pH 6 with conc. HCl. The resulting mixture was extracted with EtOAc (300 mL). The combined organic layers were washed with water (3×300 mL), dried over anhydrous Na₂SO₄. After filtration, the filtrate was concentrated under reduced pressure. This resulted in (3S)-1-[(2S)-2-[(tert-butoxycarbonyl)amino]-3-[4-[3-(3-hydroxy-2,2-dimethylpropyl)-2-iodo-1H-indol-5-yl]-1,3-thiazol-2-yl]propanoyl]-1,2-diazinane-3-carboxylic acid (25 g, crude) as a yellow oil. The crude product was used in the next step directly without further purification. LCMS (ESI): m/z [M+H] calc'd for C₂₉H₃₈IN₅O₆S 711.1; found 712.2.

### Step 9

To a stirred solution of (3S)-1-[(2S)-2-[(tert-butoxycarbonyl)amino]-3-[4-[3-(3-hydroxy-2,2-dimethylpropyl)-2-iodo-1H-indol-5-yl]-1,3-thiazol-2-yl]propanoyl]-1,2-diazinane-3-carboxylic acid (25 g, 35.13 mmol) and HOBT (23.74 g, 175.6 mmol) and DIPEA (136.21 g, 1053.9 mmol) in DCM (2 L) were added EDCl (188.5 g, 983.6 mmol) in portions at 0° C. The resulting mixture was stirred for overnight at room temperature under nitrogen atmosphere. The resulting mixture was washed with water (6 L), dried over anhydrous Na₂SO₄. After filtration, the filtrate was concentrated under reduced pressure. The residue was purified by silica gel column chromatography, eluted with PE/EtOAc (2:1) to afford tert-butyl ((6³S,4S,Z)-1²-iodo-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (13 g, 45.88%) as a yellow solid. LCMS (ESI): m/z [M+H] calc'd for C₂₉H₃₆IN₅O₅S 693.1; found 694.0.

### Step 10

To a stirred mixture of tert-butyl ((6³S,4S,Z)-1²-iodo-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (13 g, 18.7 mmol) and KOAc (6.44 g, 65.6 mmol) and s-Phos (2.31 g, 5.62 mmol) in toluene (120 mL) were added Pd₂(dba)₃ (2.06 g, 2.25 mmol) in portions at room temperature under argon atmosphere. To the stirred solution were added 4,4,5,5-tetramethyl-1,3,2-dioxaborolane (17.99 g, 140.5 mmol) dropwise at 0° C. under argon atmosphere. The resulting mixture was stirred for 3 h at 60° C. under argon atmosphere. The reaction was quenched with sat. NH₄Cl (aq.) at 0° C. The resulting mixture was concentrated under vacuum. The resulting mixture was extracted with EtOAc (200 mL). The combined organic layers were washed with water (3×300 mL), dried over anhydrous Na₂SO₄. After filtration, the filtrate was concentrated under reduced pressure. The residue was purified by silica gel column chromatography, eluted with PE/EtOAc (3:1) to afford tert-butyl ((6³S,4S,Z)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (10 g, 68.6% yield) as a yellow solid. LCMS (ESI): m/z [M+H] calc'd for C₃₅H₄₈BN₅O₇S 693.3; found 694.4.

### Intermediate D. Synthesis of(S)-3-bromo-5-iodo-2-(1-methoxyethyl)pyridine

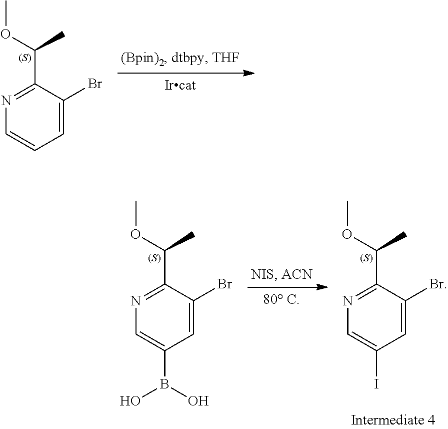

### Step 1

To a stirred solution of 3-bromo-2-[(1S)-1-methoxyethyl]pyridine (80.00 g, 370.24 mmol, 1.00 equiv) and bis(pinacolato)diboron (141.03 g, 555.3 mmol, 1.50 equiv) in THF (320 mL) was added dtbpy (14.91 g, 55.5 mmol) and Chloro(1,5-cyclooctadiene)iridium(I) dimer (7.46 g, 11.1 mmol) under argon atmosphere. The resulting mixture was stirred for 16 h at 75° C. under argon atmosphere. The mixture was concentrated under reduced pressure. The resulting mixture was dissolved in EtOAc (200 mL) and the mixture was adjusted to pH 10 with Na₂CO₃ (40 g) and NaOH (10 g) (mass 4:1) in water (600 mL). The aqueous layer was extracted with EtOAc (800 mL). The aqueous phase was acidified to pH=6 with HCl (6 N) to precipitate the desired solid to afford 5-bromo-6-[(1S)-1-methoxyethyl]pyridin-3-ylboronic acid (50 g, 52.0% yield) as a light-yellow solid. LCMS (ESI): m/z [M+H] calc'd for C₈H₁₁BBrNO₃ 259.0; found 260.0.

### Step 2

To a stirred solution of 5-bromo-6-[(1S)-1-methoxyethyl]pyridin-3-ylboronic acid (23.00 g, 88.5 mmol) in ACN (230 mL) were added NIS (49.78 g, 221.2 mmol) at room temperature under argon atmosphere. The resulting mixture was stirred for overnight at 80° C. under argon atmosphere. The resulting mixture was concentrated under reduced pressure. The resulting mixture was dissolved in DCM (2.1 L) and washed with Na₂S₂O₃ (3×500 mL). The organic layer was dried over anhydrous Na₂SO₄. After filtration, the filtrate was concentrated under reduced pressure. The residue was purified by silica gel column chromatography to afford(S)-3-bromo-5-iodo-2-(1-methoxyethyl)pyridine (20 g, 66.0% yield). LCMS (ESI): m/z [M+H] calc'd for C₈H₉BrINO 340.9; found 341.7.

### Intermediate E. Synthesis of tert-butyl ((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate

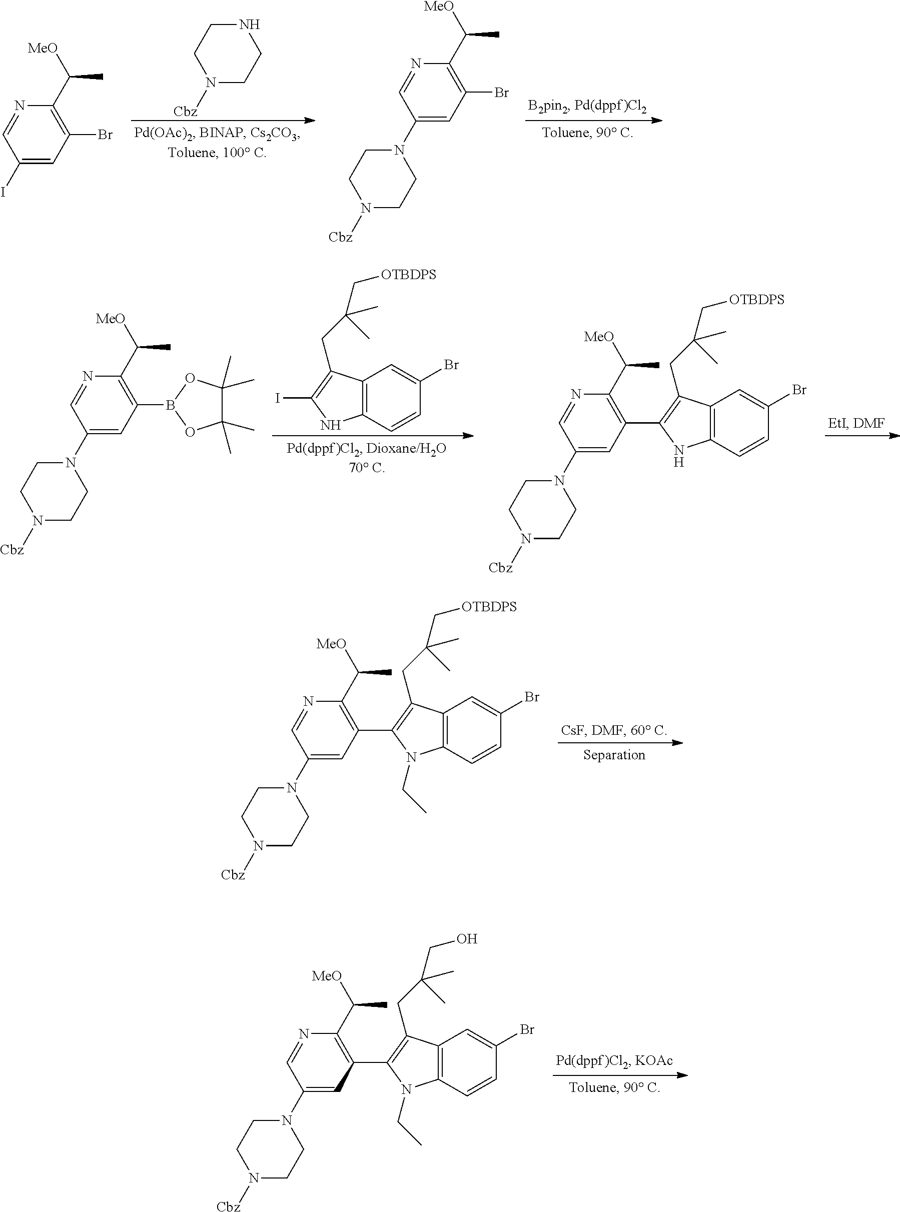

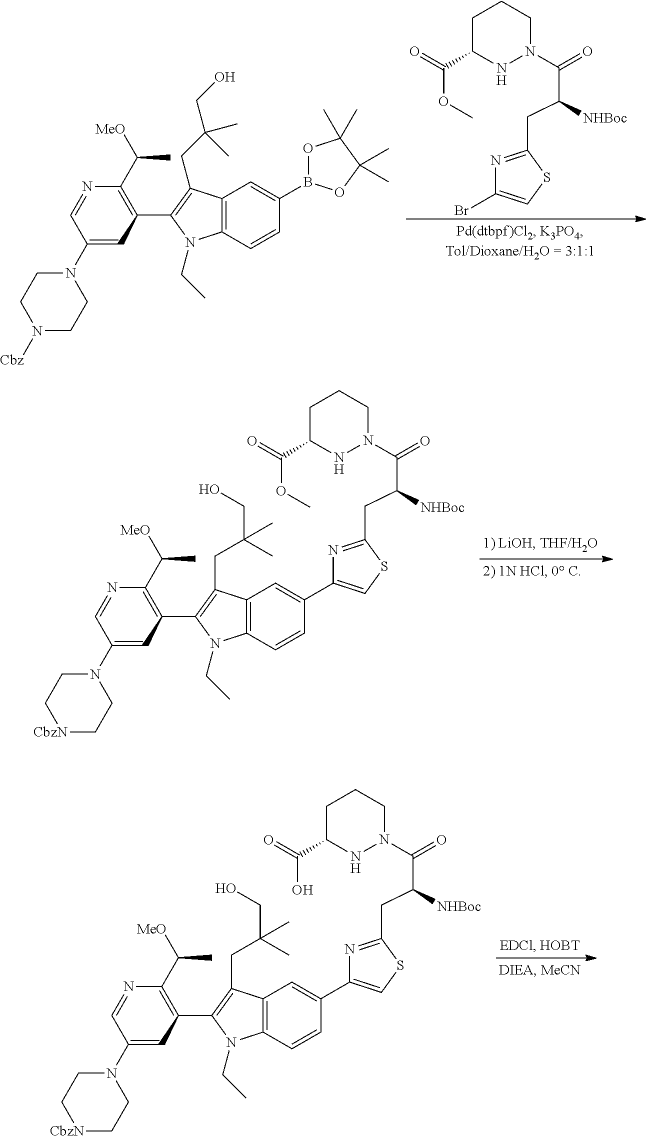

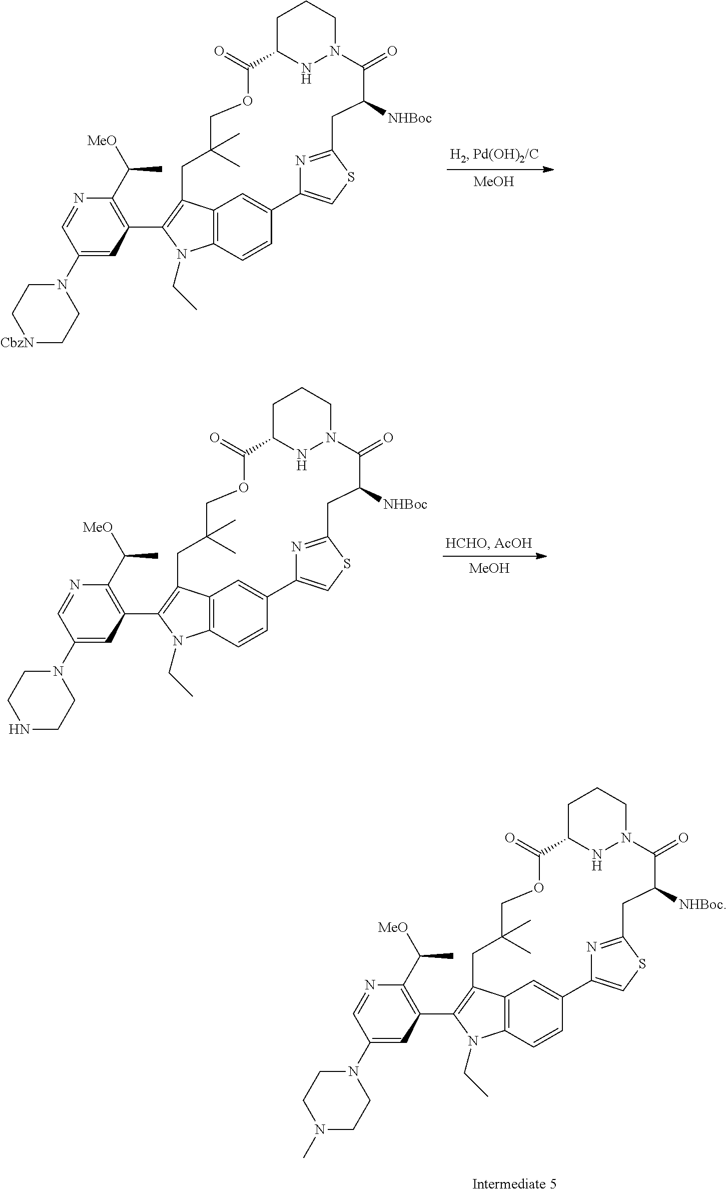

### Step 1

Into a 3 L 3-necked round-bottom flask purged and maintained with an inert atmosphere of argon, was placed 3-bromo-5-iodo-2-[(1S)-1-methoxyethyl]pyridine (147 g, 429.8 mmol) benzyl piperazine-1-carboxylate (94.69 g, 429.8 mmol), Pd(OAc)₂ (4.83 g, 21.4 mmol), BINAP (5.35 g, 8.6 mmol), Cs₂CO₃ (350.14 g, 1074.6 mmol), toluene (1 L). The resulting solution was stirred for overnight at 100° C. in an oil bath. The reaction mixture was cooled to 25° C. after reaction completed. The resulting mixture was 10 concentrated under reduced pressure. The residue was applied onto a silica gel column with ethyl acetate/hexane (1:1). Removal of solvent under reduced pressure gave benzyl(S)-4-(5-bromo-6-(1-methoxyethyl)pyridin-3-yl) piperazine-1-carboxylate (135 g, 65.1% yield) as a dark yellow solid. LCMS (ESI): m/z [M+H] calc'd for C₂₀H₂₄BrN₃O₃ 433.1; found 434.1.

### Step 2

Into a 3-L 3-necked round-bottom flask purged and maintained with an inert atmosphere of argon, was placed benzyl 4-[5-bromo-6-[(1S)-1-methoxyethyl]pyridin-3-yl]piperazine-1-carboxylate (135 g, 310.8 mmol), bis(pinacolato)diboron (86.82 g, 341.9 mmol), Pd(dppf)Cl₂ (22.74 g, 31.0 mmol), KOAc (76.26 g, 777.5 mmol), Toluene (1 L). The resulting solution was stirred for 2 days at 90° C. in an oil bath. The reaction mixture was cooled to 25° C. The resulting mixture was concentrated under vacuum. The residue was applied onto a neutral alumina column with ethyl acetate/hexane (1:3). Removal of solvent under reduced pressure gave benzyl(S)-4-(6-(1-methoxyethyl)-5-(4,4,5,5-tetramethyl-1,3,2-dioxaborolan-2-yl) pyridin-3-yl) piperazine-1-carboxylate (167 g, crude) as a dark yellow solid. LCMS (ESI): m/z [M+H] calc'd for C₂₆H₃₆BN₃O₅ 481.3; found 482.1.

### Step 3

Into a 3-L 3-necked round-bottom flask purged and maintained with an inert atmosphere of argon, was placed(S)-4-(6-(1-methoxyethyl)-5-(4,4,5,5-tetramethyl-1,3,2-dioxaborolan-2-yl) pyridin-3-yl) piperazine-1-carboxylate (167 g, 346.9 mmol), 5-bromo-3-[3-[(tert-butyldiphenylsilyl)oxy]-2,2-dimethylpropyl]-2-iodo-1H-indole (224.27 g, 346.9 mmol), Pd(dppf)Cl₂ (25.38 g, 34.6 mmol), dioxane (600 mL), H₂O (200 mL), K₃PO₄ (184.09 g, 867.2 mmol), Toluene (200 mL). The resulting solution was stirred for overnight at 70° C. in an oil bath. The reaction mixture was cooled to 25° C. after reaction completed. The resulting mixture was concentrated under vacuum. The residue was applied onto a silica gel column with ethyl acetate/hexane (1:1). Removal of solvent under reduced pressure gave benzyl(S)-4-(5-(5-bromo-3-(3-((tert-butyldiphenylsilyl)oxy)-2,2-dimethylpropyl)-1H-indol-2-yl)-6-(1-methoxyethyl)pyridin-3-yl) piperazine-1-carboxylate (146 g, 48.1% yield) as a yellow solid. LCMS (ESI): m/z [M+H] calc'd for C₄₉H₅₇BrN₄O₄Si 872.3; found 873.3.

### Step 4

To a stirred mixture of benzyl(S)-4-(5-(5-bromo-3-(3-((tert-butyldiphenylsilyl)oxy)-2,2-dimethylpropyl)-1H-indol-2-yl)-6-(1-methoxyethyl)pyridin-3-yl) piperazine-1-carboxylate (146 g, 167.0 mmol) and Cs₂CO₃ (163.28 g, 501.1 mmol) in DMF (1200 mL) was added C₂H₅I (52.11 g, 334.0 mmol) in portions at 0° C. under N₂ atmosphere. The final reaction mixture was stirred at 25° C. for 12 h. Desired product could be detected by LCMS. The resulting mixture was diluted with EA (1 L) and washed with brine (3×1.5 L). The organic layers were dried over anhydrous Na₂SO₄. After filtration, the filtrate was concentrated under reduced pressure to give benzyl(S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl) piperazine-1-carboxylate (143 g, crude) as a yellow solid that was used directly for next step without further purification. LCMS (ESI): m/z [M+H] calc'd for C₅₁H₆₁BrN₄O₄Si 900.4; found 901.4.

### Step 5

To a stirred mixture of benzyl benzyl(S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl) piperazine-1-carboxylate (143 g, 158.5 mmol) in DMF (1250 mL) was added CsF (72.24 g, 475.5 mmol). Then the reaction mixture was stirred at 60° C. for 2 days under N₂ atmosphere. Desired product could be detected by LCMS. The resulting mixture was diluted with EA (1 L) and washed with brine (3×1 L). Then the organic phase was concentrated under reduced pressure. The residue was purified by silica gel column chromatography, eluted with PE/EA (1/3) to afford two atropisomers of benzyl(S)-4-(5-(5-bromo-1-ethyl-3-(3-hydroxy-2,2-dimethylpropyl)-1H-indol-2-yl)-6-(1-methoxyethyl)pyridin-3-yl) piperazine-1-carboxylate A (38 g, 36% yield, RT=1.677 min in 3 min LCMS (0.1% FA)) and B (34 g, 34% yield, RT=1.578 min in 3 min LCMS (0.1% FA)) both as yellow solid. LCMS (ESI): m/z [M+H] calc'd for C₃₅H₄₃BrN₄O₄ 663.2; found 662.2.

### Step 6

Into a 500-mL 3-necked round-bottom flask purged and maintained with an inert atmosphere of nitrogen, was placed benzyl(S)-4-(5-(5-bromo-1-ethyl-3-(3-hydroxy-2,2-dimethylpropyl)-1H-indol-2-yl)-6-(1-methoxyethyl)pyridin-3-yl) piperazine-1-carboxylate A (14 g, 21.1 mmol), bis(pinacolato)diboron (5.89 g, 23.21 mmol), Pd(dppf)Cl₂ (1.54 g, 2.1 mmol), KOAc (5.18 g, 52.7 mmol), Toluene (150 mL). The resulting solution was stirred for 5 h at 90° C. in an oil bath. The reaction mixture was cooled to 25° C. The resulting mixture was concentrated under vacuum. The residue was purified by silica gel column chromatography, eluted with PE/EA (1/3) to give benzyl(S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl) piperazine-1-carboxylate (12 g, 76.0% yield) as a yellow solid. LCMS (ESI): m/z [M+H] calc'd for C₄₁H₅₅BN₄O₆ 710.4; found 711.3.

### Step 7

Into a 250-mL round-bottom flask purged and maintained with an inert atmosphere of argon, was placed benzyl(S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl) piperazine-1-carboxylate (10.8 g, 15.2 mmol), methyl (3S)-1-[(2S)-3-(4-bromo-1,3-thiazol-2-yl)-2-[(tert-butoxycarbonyl)amino]propanoyl]-1,2-diazinane-3-carboxylate (7.98 g, 16.7 mmol), Pd(dtbpf)Cl₂ (0.99 g, 1.52 mmol), K₃PO₄ (8.06 g, 37.9 mmol), Toluene (60 mL), dioxane (20 mL), H₂O (20 mL). The resulting solution was stirred for 3 h at 70° C. in an oil bath. The reaction mixture was cooled to 25° C. The resulting solution was extracted with EtOAc (2×50 mL) and concentrated under reduced pressure. The residue was applied onto a silica gel column with ethyl acetate/hexane (10:1). Removal of solvent to give methyl(S)-1-((S)-3-(4-(2-(5-(4-((benzyloxy) carbonyl) piperazin-1-yl)-2-((S)-1-methoxyethyl)pyridin-3-yl)-1-ethyl-3-(3-hydroxy-2,2-dimethylpropyl)-1H-indol-5-yl) thiazol-2-yl)-2-((tert-butoxycarbonyl)amino) propanoyl) hexahydropyridazine-3-carboxylate (8 g, 50.9% yield) as a yellow solid. LCMS (ESI): m/z [M+H] calc'd for C₅₂H₆₈N₈O₉S 980.5; found 980.9.

### Step 8

To a stirred mixture of methyl(S)-1-((S)-3-(4-(2-(5-(4-((benzyloxy) carbonyl) piperazin-1-yl)-2-((S)-1-methoxyethyl)pyridin-3-yl)-1-ethyl-3-(3-hydroxy-2,2-dimethylpropyl)-1H-indol-5-yl) thiazol-2-yl)-2-((tert-butoxycarbonyl)amino) propanoyl) hexahydropyridazine-3-carboxylate (12 g, 12.23 mmol) in THF (100 mL)/H₂O (100 mL) was added LiOH (2.45 g, 61.1 mmol) under N₂ atmosphere and the resulting mixture was stirred for 2 h at 25° C. Desired product could be detected by LCMS. THF was concentrated under reduced pressure. The pH of aqueous phase was acidified to 5 with HCL (1N) at 0° C. The aqueous layer was extracted with DCM (3×100 ml). The organic phase was concentrated under reduced pressure to give(S)-1-((S)-3-(4-(2-(5-(4-((benzyloxy) carbonyl) piperazin-1-yl)-2-((S)-1-methoxyethyl)pyridin-3-yl)-1-ethyl-3-(3-hydroxy-2,2-dimethylpropyl)-1H-indol-5-yl) thiazol-2-yl)-2-((tert-butoxycarbonyl)amino) propanoyl) hexahydropyridazine-3-carboxylic acid (10 g, 84.5% yield) as a light yellow solid. LCMS (ESI): m/z [M+H] calc'd for C₅₁H₆₆N₈O₉S 966.5; found 967.0.

### Step 9

Into a 3-L round-bottom flask purged and maintained with an inert atmosphere of nitrogen, was placed(S)-1-((S)-3-(4-(2-(5-(4-((benzyloxy) carbonyl) piperazin-1-yl)-2-((S)-1-methoxyethyl)pyridin-3-yl)-1-ethyl-3-(3-hydroxy-2,2-dimethylpropyl)-1H-indol-5-yl) thiazol-2-yl)-2-((tert-butoxycarbonyl)amino) propanoyl) hexahydropyridazine-3-carboxylic acid (18 g, 18.61 mmol), ACN (1.8 L), DIEA (96.21 g, 744.4 mmol), EDCl (107.03 g, 558.3 mmol), HOBT (25.15 g, 186.1 mmol). The resulting solution was stirred for overnight at 25° C. The resulting mixture was concentrated under vacuum after reaction completed. The resulting solution was diluted with DCM (1 L). The resulting mixture was washed with HCl (3×1 L, IN aqueous). The resulting mixture was washed with water (3×1 L). Then the organic layer was concentrated, the residue was applied onto a silica gel column with ethyl acetate/hexane (1:1). Removal of solvent under reduced pressure gave benzyl 4-(5-((6³S,4S,Z)-4-((tert-butoxycarbonyl)amino)-11-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl) piperazine-1-carboxylate (10.4 g, 54.8% yield) as a light yellow solid. LCMS (ESI): m/z [M+H] calc'd for C₅₁H₆₄N₈O₈S 948.5; found 949.3.

### Step 10

Into a 250-mL round-bottom flask purged and maintained with an inert atmosphere of nitrogen, was placed benzyl 4-(5-((6³S,4S,Z)-4-((tert-butoxycarbonyl)amino)-11-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl) piperazine-1-carboxylate (10.40 g, 10.9 mmol), Pd(OH)₂/C (5 g, 46.9 mmol), MeOH (100 mL). The resulting solution was stirred for 3 h at 25° C. under 2 atm H₂ atmosphere. The solids were filtered out and the filter cake was washed with MeOH (3×100 mL). Then combined organic phase was concentrated under reduced pressure to give tert-butyl ((6³S,4S,Z)-1¹-ethyl-1²-(2-((S)-1-methoxyethyl)-5-(piperazin-1-yl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (8.5 g, 90.4% yield) as a light yellow solid. LCMS (ESI): m/z [M+H] calc'd for C₄₃H₅₈N₈O₆S 814.4; found 815.3.

### Step 11

Into a 1000-mL round-bottom flask purged and maintained with an inert atmosphere of nitrogen, was placed tert-butyl ((6³S,4S,Z)-1¹-ethyl-1²-(2-((S)-1-methoxyethyl)-5-(piperazin-1-yl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (8.5 g, 10.4 mmol), MeOH (100 mL), AcOH (1.88 g, 31.2 mmol) and stirred for 15 mins. Then HCHO (1.88 g, 23.15 mmol, 37% aqueous solution) and NaBH₃CN (788 mg, 12.5 mmol) was added at 25° C. The resulting solution was stirred for 3 h at 25° C. The resulting mixture was quenched with 100 ml water and concentrated under reduced pressure to remove MeOH. The resulting solution was diluted with 300 mL of DCM. The resulting mixture was washed with water (3×100 mL). Removal of solvent gave tert-butyl ((6³S,4S,Z)-1¹-ethyl-1²-(2-((S)-1-methoxyethyl)-5-(4-methylpiperazin-1-yl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (8.2 g, 90.1% yield) as a yellow solid. LCMS (ESI): m/z [M+H] calc'd for C₄₄H₆₀N₈O₆S 828.4; found 829.3.

### Intermediate 1. Synthesis of(S)-(2-(5-(4-cyclopropylpiperazin-1-yl)-2-(1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) boronic acid

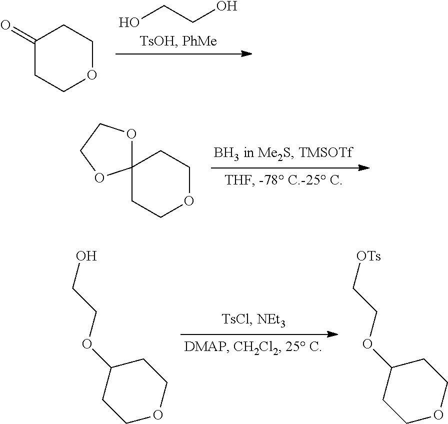

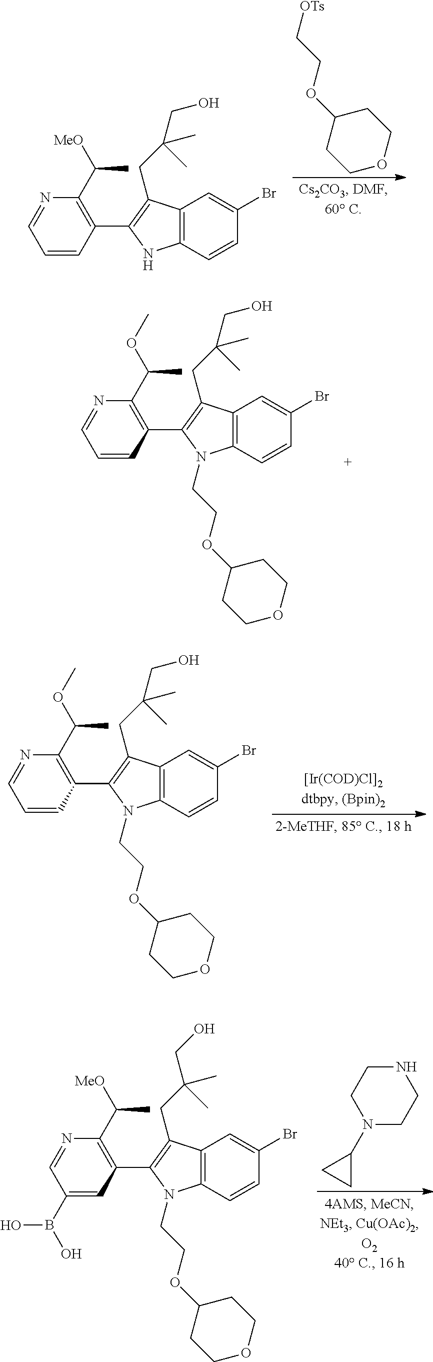

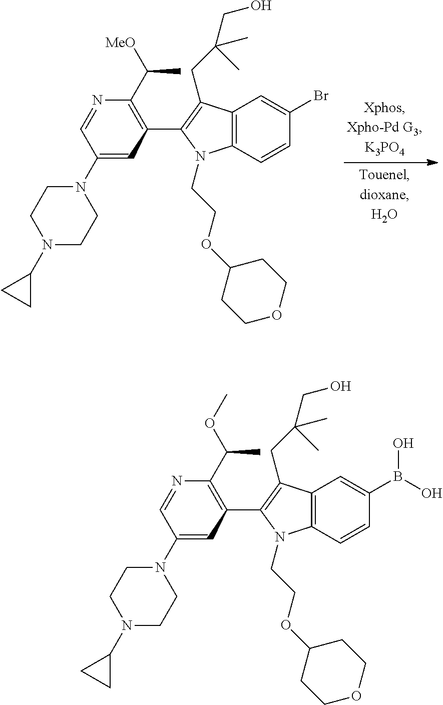

### Step 1

To a stirred solution of tetrahydro-4H-pyran-4-one (400 g, 3995.325 mmol) and TsOH (68.8 g, 399.53 mmol) in toluene (2 L) was added ethylene glycol (248 g, 3995.32 mmol) dropwise at 20° C. under a nitrogen atmosphere. The reaction needed a water knockout vessel to remove H₂O. The resulting mixture was stirred for 3 h at 100° C. The resulting mixture was washed with saturated NaHCO₃(aq). The resulting mixture was extracted with EtOAc (3×1 L). The combined organic layers were washed with brine (2×1 L), dried over anhydrous Na₂SO₄. After filtration, the filtrate was concentrated under reduced pressure. The crude product (267 g, crude) was used in the next step directly without further purification. LCMS (ESI): m/z [M+H] calc'd for C₇H₁₃O₃ 145.1; found 145.1.

### Step 2

To a stirred solution of 1,4,8-trioxaspiro[4.5]decane (280 g, 1942.15 mmol) in THF (2800 mL) was added BH₃-Me₂S (233.1 mL, 2330.6 mmol) dropwise at −78° C. under nitrogen atmosphere. To the above mixture was added TMSOTf (21.58 g, 97.11 mmol) dropwise over 15 min at −70° C. The resulting mixture was stirred for additional 16 h at 20° C. The reaction was quenched with MeOH (145 g) added dropwise at 0° C. and was stirred for 3 h at 25° C. The resulting mixture was concentrated under reduced pressure. The crude product (250 g, crude) was used in the next step directly without further purification. LCMS (ESI): m/z [M+H] calc'd for C₇H₁₅O₃ 147.1; found 147.1.

### Step 3

To a stirred solution of 2-(oxan-4-yloxy)ethanol (250 g, 1710.15 mmol) in CH₂Cl₂ (1.5 L) were added NEt₃ (519.17 g, 5130.45 mmol) and DMAP (20.89 g, 171.02 mmol) dropwise at 0° C. under a nitrogen atmosphere. To the above mixture was added TsCl (391.23 g, 2052.19 mmol) in CH₂Cl₂ (1 L) dropwise over 15 min at 0° C. The resulting mixture was stirred for additional 3 h at room temperature. The mixture was neutralized to pH 7 with 1M HCl(aq). The resulting mixture was extracted with CH₂Cl₂ (3×1000 mL). The combined organic layers were washed with brine (3×1500 mL), dried over anhydrous Na₂SO₄. After filtration, the filtrate was concentrated under reduced pressure. The residue was purified by silica gel column chromatography, eluted with PE/EtOAc (8:1) to afford 2-((tetrahydro-2H-pyran-4-yl)oxy)ethyl 4-methylbenzenesulfonate (220 g, 42.8%) as a brown oil. LCMS (ESI): m/z [M+H] calc'd for C₁₄H₂₀O₅S 301.1; found 301.0.

### Step 4

To a stirred solution of 3-(5-bromo-2-{2-[(1S)-1-methoxyethyl]pyridin-3-yl}-1H-indol-3-yl)-2,2-dimethylpropan-1-ol (220 g, 527.14 mmol) in DMF (1600 mL) were added Cs₂CO₃ (429.38 g, 1317.85 mmol) and 2-((tetrahydro-2H-pyran-4-yl)oxy)ethyl 4-methylbenzenesulfonate (221.67 g, 738.0 mmol) in portions at 25° C. under nitrogen atmosphere. The resulting mixture was stirred for 16 h at 60° C. The resulting mixture was diluted with H₂O (3000 mL). The resulting mixture was extracted with EtOAc (3×2000 mL). The combined organic layers were washed with brine (3×2000 mL), dried over anhydrous Na₂SO₄ and filtered. After filtration, the filtrate was concentrated under reduced pressure. The residue was purified by silica gel column chromatography, eluted with PE/EtOAc (1:1) to afford the mixture (160 g) of(S)-3-(5-bromo-2-(2-(1-methoxyethyl)pyridin-3-yl)-1-(2-((tetrahydro-2H-pyran-4-yl)oxy)ethyl)-1H-indol-3-yl)-2,2-dimethylpropan-1-ol A and (S)-3-(5-bromo-2-(2-(1-methoxyethyl)pyridin-3-yl)-1-(2-((tetrahydro-2H-pyran-4-yl)oxy)ethyl)-1H-indol-3-yl)-2,2-dimethylpropan-1-ol B with the ratio of 7:3 and eluted with EA to afford(S)-3-(5-bromo-2-(2-(1-methoxyethyl)pyridin-3-yl)-1-(2-((tetrahydro-2H-pyran-4-yl)oxy)ethyl)-1H-indol-3-yl)-2,2-dimethylpropan-1-ol B (156 g, 54.2%). The mixture (160 g) could be washed with MTBE (160 ml) to afford the pure(S)-3-(5-bromo-2-(2-(1-methoxyethyl)pyridin-3-yl)-1-(2-((tetrahydro-2H-pyran-4-yl)oxy)ethyl)-1H-indol-3-yl)-2,2-dimethylpropan-1-ol A (112 g, 39.0%). LCMS (ESI): m/z [M+H] calc'd for C₂₈H₃₇BrN₂O₄ 545.2; found 545.1.

### Step 5

To a stirred mixture of(S)-3-(5-bromo-2-(2-(1-methoxyethyl)pyridin-3-yl)-1-(2-((tetrahydro-2H-pyran-4-yl)oxy)ethyl)-1H-indol-3-yl)-2,2-dimethylpropan-1-ol A (100 g, 183.31 mmol) and 4,4,5,5-tetramethyl-2-(tetramethyl-1,3,2-dioxaborolan-2-yl)-1,3,2-dioxaborolane (93.10 g, 366.62 mmol) in 2-methyltetrahydrofuran (400 mL) were added dtbpy (7.38 g, 27.5 mmol) and bis((1Z,5Z)-cycloocta-1,5-diene); bis(chloroiridium) (2.46 g, 3.67 mmol) in portions at 25° C. under argon atmosphere. The resulting mixture was stirred for 16 h at 85° C. under an argon atmosphere. The mixture was acidified to pH 6 with HCl (aq.). The resulting mixture was extracted with CH₂Cl₂ (3×800 mL). The combined organic layers were washed with brine (3×800 mL), dried over anhydrous Na₂SO₄. After filtration, the filtrate was concentrated under reduced pressure. This resulted in (S)-(5-(5-bromo-3-(3-hydroxy-2,2-dimethylpropyl)-1-(2-((tetrahydro-2H-pyran-4-yl)oxy)ethyl)-1H-indol-2-yl)-6-(1-methoxyethyl)pyridin-3-yl) boronic acid (130 g, crude) as a light red solid. ESI-MS m/z=589.3, 591.3 [M+H]⁺; Calculated MW: 588.2, 590.2. LCMS (ESI): m/z [M+H] calc'd for C₂₈H₃₈BBrN₂O₆ 589.21; found 591.3.

### Step 6

To a stirred mixture of(S)-(5-(5-bromo-3-(3-hydroxy-2,2-dimethylpropyl)-1-(2-((tetrahydro-2H-pyran-4-yl)oxy)ethyl)-1H-indol-2-yl)-6-(1-methoxyethyl)pyridin-3-yl) boronic acid (130 g, 193.60 mmol) and 4A MS (130 g) in MeCN (3250 mL) were added NEt₃ (39.18 g, 387.20 mmol) and 1-cyclopropylpiperazine (73.30 g, 580.81 mmol) in portions at 25° C. under an oxygen atmosphere. To the above mixture was added Cu(OAc)₂ (52.75 g, 290.40 mmol) in portions over 30 min at 25° C. The resulting mixture was stirred for additional 16 h at 40° C. The mixture was filtered, the filter cake was washed with EtOAc (3×600 mL). The filtrate was concentrated under reduced pressure. The residue was extracted with EtOAc (3×800 mL). The combined organic layers were washed with brine (3×800 mL), dried over anhydrous Na₂SO₄. After filtration, the filtrate was concentrated under reduced pressure. The residue was purified by silica gel column chromatography, eluted with PE/EtOAc (1:1) to afford(S)-3-(5-bromo-2-(5-(4-cyclopropylpiperazin-1-yl)-2-(1-methoxyethyl)pyridin-3-yl)-1-(2-((tetrahydro-2H-pyran-4-yl)oxy)ethyl)-1H-indol-3-yl)-2,2-dimethylpropan-1-ol (78 g, 60.2%) as a brown oil. LCMS (ESI): m/z [M+H] calc'd for C₃₅H₅₀BrN₄O₄ 669.30; found 669.4.

### Step 7

To a stirred solution of(S)-3-(5-bromo-2-(5-(4-cyclopropylpiperazin-1-yl)-2-(1-methoxyethyl)pyridin-3-yl)-1-(2-((tetrahydro-2H-pyran-4-yl)oxy)ethyl)-1H-indol-3-yl)-2,2-dimethylpropan-1-ol (65 g, 97.06 mmol) and XPhos Pd G3 (2.46 g, 2.91 mmol) in 2-methyltetrahydrofuran (650 mL) and MeOH (320 mL) were added B₂(OH)₄ (10.44 g, 116.47 mmol) and potassium 2,2-dimethylpropanoate (27.22 g, 194.12 mmol) in portions at 40° C. under a nitrogen atmosphere. Desired product could be detected by LCMS. The resulting mixture was filtered; the filter cake was washed with EtOAc (6×100 mL). The filtrate was concentrated under reduced pressure. The resulting mixture was washed with H₂O (1×500 mL). The aqueous layer was extracted with EtOAc (1×300 mL). The resulting mixture was washed with brine (1×500 mL). The crude product was used in the next step directly without further purification. This resulted in(S)-(2-(5-(4-cyclopropylpiperazin-1-yl)-2-(1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) boronic acid (79 g, crude) as a white solid. LCMS (ESI): m/z [M+H] calc'd for C₃₅H₅₂BN₄O₆ 635.40; found 635.7.

### Intermediate 2. Synthesis of (2S,3S)-3-(4-bromothiazol-2-yl)-2-((tert-butoxycarbonyl)amino)-3-(2-oxa-6-azaspiro[3.3]heptan-6-yl) propanoic acid

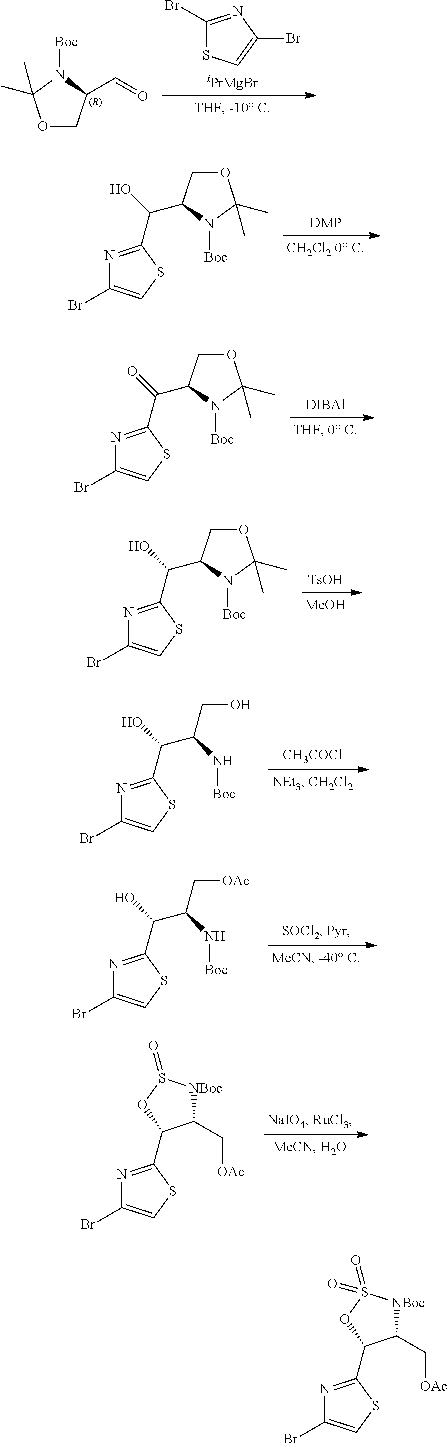

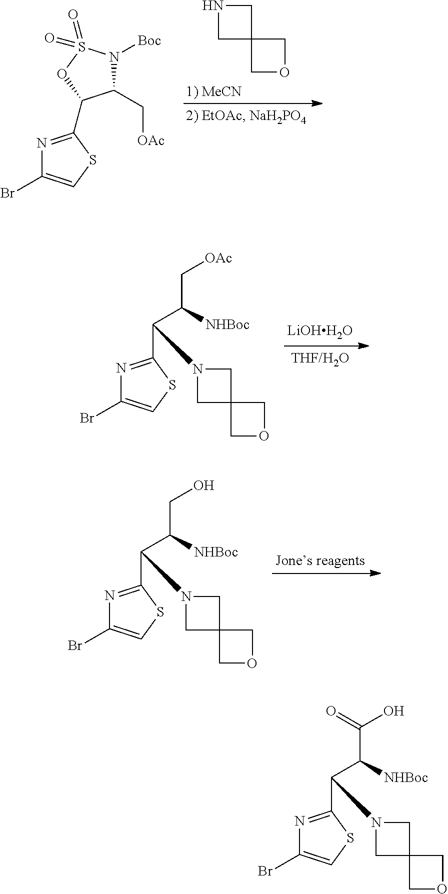

### Step 1

A solution of 2,4-dibromo-1,3-thiazole (23.31 g, 95.95 mmol) in THF (250 mL) was treated with iPr-MgBr (96 mL, 1M in THF) at −10° C. under a nitrogen atmosphere and stirred for 1 h. Then the dropwise addition of tert-butyl (4R)-4-formyl-2,2-dimethyl-1,3-oxazolidine-3-carboxylate (20 g, 87.23 mmol) in 50 mL THF at −10° C. The resulting mixture was diluted with H₂O (500 mL). The aqueous layer was extracted with EtOAc (3×300 mL). The resulting organic phase was concentrated under reduced pressure. The residue was purified by silica gel column chromatography, eluted with PE/EtOAc (10:1) to afford tert-butyl (4R)-4-((4-bromothiazol-2-yl) (hydroxy)methyl)-2,2-dimethyloxazolidine-3-carboxylate (31.6 g, 92.11%) as colorless oil. LCMS (ESI): m/z [M+H] calc'd for C₁₄H₂₂BrN₂O₄S 393.05; found 393.1.

### Step 2

Into a 2000 mL 2-necked round-bottom flask were added tert-butyl (4R)-4-((4-bromothiazol-2-yl) (hydroxy)methyl)-2,2-dimethyloxazolidine-3-carboxylate (32.6 g, 82.89 mmol) and DMP (70.31 g, 165.78 mmol) in CH₂Cl₂ (500 mL) at 0° C. The resulting mixture was stirred for 4 h at 25° C. The reaction was quenched with NaHCO₃(aq.) (500 mL) at 0° C. The aqueous layer was extracted with CH₂Cl₂ (2×500 mL). The resulting organic phase was concentrated under reduced pressure. The residue was purified by silica gel column chromatography, eluted with PE/EtOAc (20:1) to afford tert-butyl (R)-4-(4-bromothiazole-2-carbonyl)-2,2-dimethyloxazolidine-3-carboxylate (29 g, 89.42%) as a colorless oil. LCMS (ESI): m/z [M+H] calc'd for C₁₄H₂₀BrN₂O₄S 391.03; found 391.1.

### Step 3

A solution of tert-butyl (R)-4-(4-bromothiazole-2-carbonyl)-2,2-dimethyloxazolidine-3-carboxylate (29 g, 74.12 mmol) in THF (500 mL) was added DIBAL (111.17 mL, 111.17 mmol) dropwise over 1 h at 0° C. under a nitrogen atmosphere. The reaction was quenched with NH₄Cl (aq.) (500 mL) at 0° C. The precipitated solids were filtrated and washed with EtOAc (5×200 mL). The resulting organic phase was concentrated under reduced pressure. The crude product (26 g) was used in the next step directly without further purification. LCMS (ESI): m/z [M+H] calc'd for C₁₄H₂₂BrN₂O₄S 393.05; found 393.3.

### Step 4

To a solution of tert-butyl (R)-4-((R)-(4-bromothiazol-2-yl) (hydroxy)methyl)-2,2-dimethyl-1|3-oxazolidine-3-carboxylate (29.6 g, 75.26 mmol) in MeOH (500 mL) was added TsOH (1.30 g, 7.53 mmol) under a nitrogen atmosphere. The resulting mixture was stirred for overnight at 40° C. The mixture was concentrated under reduced pressure. The residue was purified by silica gel column chromatography, eluted with PE/EtOAc (2:1) to afford tert-butyl ((1R,2R)-1-(4-bromothiazol-2-yl)-1,3-dihydroxypropan-2-yl) carbamate (16 g, 57.18%) as a white solid. LCMS (ESI): m/z [M+H] calc'd for C₁₁H₁₈BrN₂O₄S 353.02; found 353.2.

### Step 5

To a solution of tert-butyl ((1R,2R)-1-(4-bromothiazol-2-yl)-1,3-dihydroxypropan-2-yl) carbamate (5 g, 14.16 mmol) in CH₂Cl₂ (100 mL) was added NEt₃ (4.30 g, 42.47 mmol) followed by the dropwise addition of AcCl (1.11 g, 14.16 mmol) in CH₂Cl₂ (20 mL) at 0° C. and stirred overnight at room temperature. The reaction was quenched with 50 mL water. The aqueous layer was extracted with CH₂Cl₂ (3×30 mL). The resulting mixture was concentrated under reduced pressure. The residue was purified by silica gel column chromatography, eluted with PE/EtOAc (5:1) to afford (2R,3R)-3-(4-bromothiazol-2-yl)-2-((tert-butoxycarbonyl)amino)-3-hydroxypropyl acetate (4 g, 67.92%) as colorless oil. LCMS (ESI): m/z [M+H] calc'd for C₁₃H₂₀BrN₂O₅S 395.03; found 395.1.

### Step 6

To a solution of SOCl₂ (3.01 g, 25.30 mmol) in MeCN (20 mL) and CH₂Cl₂ (20 mL) was added (2R,3R)-3-(4-bromothiazol-2-yl)-2-((tert-butoxycarbonyl)amino)-3-hydroxypropyl acetate (4 g, 10.12 mmol) at −40° C. under a nitrogen atmosphere and stirred for 10 mins. Pyridine (3.20 g, 40.48 mmol) in MeCN/CH₂Cl₂ (5 mL/5 mL) was added dropwise at −40° C. and stirred for 1 h. TLC show no SM remained. The resulting mixture was washed with H₂O (20 mL), and 1M HCl(aq) (20 mL). The resulting organic phase was concentrated under reduced pressure. The crude product was used in the next step directly without further purification.

### Step 7

To a solution of tert-butyl (4R,5R)-4-(acetoxymethyl)-5-(4-bromothiazol-2-yl)-1,2,3-oxathiazolidine-3-carboxylate 2-oxide (5 g, 11.33 mmol) in MeCN/H₂O (40 mL/40 mL) was added RuCl₃·H₂O (0.05 g, 0.23 mmol) at 0° C. under a nitrogen atmosphere followed by the addition of NaIO₄ (2.91 g, 13.60 mmol) in portions. The resulting mixture was stirred for overnight at 25° C. The resulting mixture was diluted with EtOAc (100 mL). The residue was washed with H₂O (3×50 mL). The resulting organic phase was concentrated under reduced pressure. The residue was purified by silica gel column chromatography, eluted with PE/EtOAc (7:1) to afford tert-butyl (4R,5R)-4-(acetoxymethyl)-5-(4-bromothiazol-2-yl)-1,2,3-oxathiazolidine-3-carboxylate 2,2-dioxide (3 g, 57.90%) as a white solid. LCMS (ESI): m/z [M+H] calc'd for C₁₃H₁₈BrN₂O₇S₂ 456.97; found 457.0.

### Step 8

To a stirred solution of tert-butyl (4R,5R)-4-(acetoxymethyl)-5-(4-bromothiazol-2-yl)-1,2,3-oxathiazolidine-3-carboxylate 2,2-dioxide (1500 g, 3280.05 mmol) was added 2-oxa-6-azaspiro[3.3]heptane (812.90 g, 8200.13 mmol) in MeCN (15000 mL) at 25° C. under an air atmosphere. The resulting mixture was concentrated under reduced pressure. The crude residue was added to a stirred mixture of NaH₂PO₄(aq) in EtOAc at 50° C. under an air atmosphere. The aqueous layer was extracted with EtOAc (3×1500 mL). The resulting mixture was concentrated under reduced pressure. The residue was washed with PE/EtOAc (85:15) (2000 mL) to afford (2S,3S)-3-(4-bromothiazol-2-yl)-2-((tert-butoxycarbonyl)amino)-3-(2-oxa-6-azaspiro[3.3]heptan-6-yl) propyl acetate (937 g, 59.9%) as a yellow solid. LCMS (ESI): m/z [M+H] calc'd for C₁₈H₂₇BrN₃₀₅S 476.09; found 475.95.

### Step 9

To a stirred solution of (2S,3S)-3-(4-bromothiazol-2-yl)-2-((tert-butoxycarbonyl)amino)-3-(2-oxa-6-azaspiro[3.3]heptan-6-yl) propyl acetate (996 g, 2090.72 mmol) in MeOH (5000 mL) and THF (5000 mL) was added KOH (aq) (350 g, in 5000 mL H₂O) dropwise at 0° C. under an air atmosphere and stirred for 2 h at 25° C. Desired product could be detected by LCMS. The pH value of reaction mixture was neutralized to 7˜8 with 1M NaH₂PO₄ (6×3000 mL). The combined mixture were concentrated under reduced pressure to a crude mixture and then extracted with EtOAc (3×3000 mL), dried over anhydrous Na₂SO₄. After filtration, the filtrate was concentrated under reduced pressure. This resulted in tert-butyl ((1S,2S)-1-(4-bromothiazol-2-yl)-3-hydroxy-1-(2-oxa-6-azaspiro[3.3]heptan-6-yl) propan-2-yl) carbamate (889.2 g, 83.23%) as a white solid. LCMS (ESI): m/z [M+H] calc'd for C₁₆H₂₃BrN₃₀₅S; found 434.15.

### Step 10

To a stirred solution of tert-butyl ((1S,2S)-1-(4-bromothiazol-2-yl)-3-hydroxy-1-(2-oxa-6-azaspiro[3.3]heptan-6-yl) propan-2-yl) carbamate (345 g, 794.29 mmol) in acetone (320 mL) was added JONE'S REAGENT (75 mL) dropwise at 0° C. under an air atmosphere and stirred for 2 h at room temperature. Desired product could be detected by LCMS. The residue was purified by Prep-HPLC. The pH of desired fraction was acidified with 1M critic acid to pH 5 and extracted with CHCI3/IPA (3:1, 5×3000 mL). The organic phase was concentrated under reduced pressure. This resulted in (2S,3S)-3-(4-bromothiazol-2-yl)-2-((tert-butoxycarbonyl)amino)-3-(2-oxa-6-azaspiro[3.3]heptan-6-yl) propanoic acid (186 g, 53.4%) as a green solid. LCMS (ESI): m/z [M+H] calc'd for C₁₆H₂₃BrN₃₀₅S 448.05; found 448.15.

### Example 1: Synthesis of (1r,2R,3S)—N-((6³S,3S,4S,Z)-1²-(5-(4-cyclopropylpiperazin-1-yl)-2-((S)-1-methoxyethyl)pyridin-3-yl)-10,10-dimethyl-5,7-dioxo-3-(2-oxa-6-azaspiro[3.3]heptan-6-yl)-11-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2,3-dimethylcyclopropane-1-carboxamide

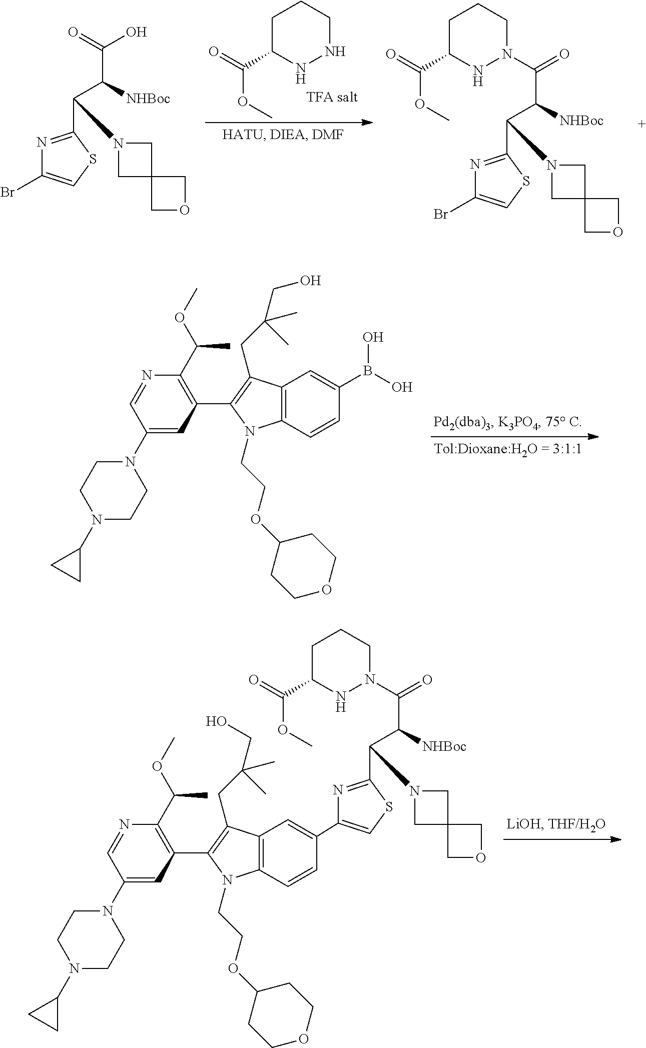

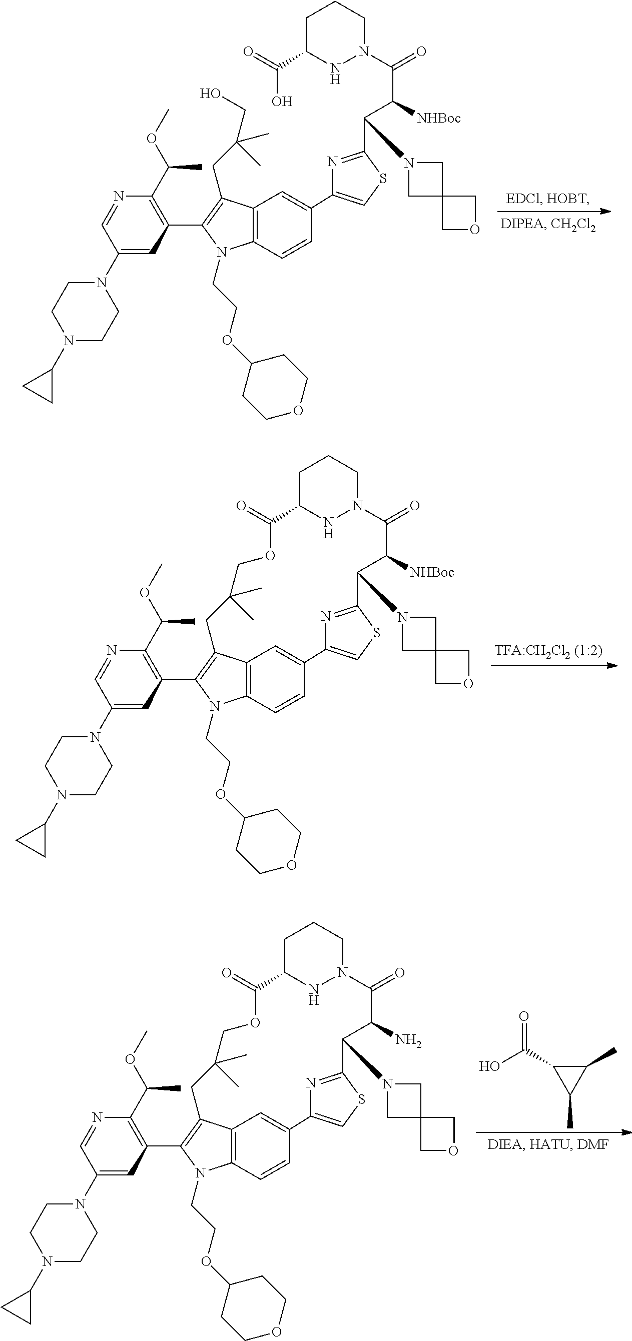

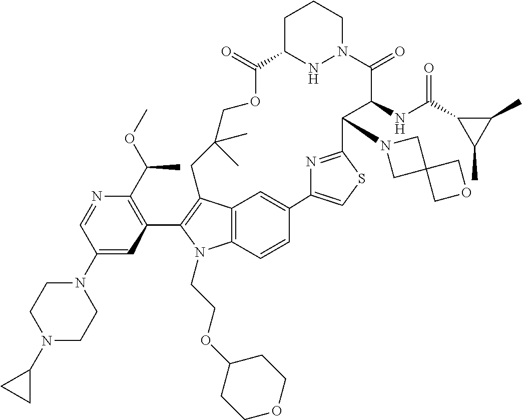

### Step 1

To a stirred solution of (2S,3S)-3-(4-bromothiazol-2-yl)-2-((tert-butoxycarbonyl)amino)-3-(2-oxa-6-azaspiro[3.3]heptan-6-yl) propanoic acid (5 g, 11.15 mmol) in DMF (50 mL) was added DIPEA (28.83 g, 223.04 mmol) dropwise at 0° C. under an air atmosphere and stirred for 5 min. methyl(S)-hexahydropyridazine-3-carboxylate (5 g, 11.15 mmol) was added into the solution. HATU (6.36 g, 16.73 mmol) was added into the solution. Desired product could be detected by LCMS. The resulting mixture was washed with H₂O (3×100 mL). The aqueous layer was extracted with EtOAc (1×100 mL). The organic layers were combined and concentrated under reduced pressure. The residue was purified by silica gel column chromatography, eluted with PE/EtOAc (1:1) to afford methyl(S)-1-((2S,3S)-3-(4-bromothiazol-2-yl)-2-((tert-butoxycarbonyl)amino)-3-(2-oxa-6-azaspiro[3.3]heptan-6-yl) propanoyl) hexahydropyridazine-3-carboxylate (4.68 g, 65.74%) as a yellow solid. LCMS (ESI): m/z [M+H] calc'd for C₂₂H₃₃BrN₅O₆S 574.13; found 573.95.

### Step 2

To a stirred solution of(S)-(2-(5-(4-cyclopropylpiperazin-1-yl)-2-(1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) boronic acid (5.2 g, 8.19 mmol) and methyl(S)-1-((2S,3S)-3-(4-bromothiazol-2-yl)-2-((tert-butoxycarbonyl)amino)-3-(2-oxa-6-azaspiro[3.3]heptan-6-yl) propanoyl) hexahydropyridazine-3-carboxylate (4.59 g, 8.19 mmol) in toluene (45 mL) and 1,4-dioxane (15 mL) and H₂O (15 mL) was added K₃PO₄ (4.35 g, 20.49 mmol) and XPhos (1.95 g, 4.10 mmol) and Pd₂(dba)₃ (2.25 g, 2.46 mmol) in portions at 75° C. under a nitrogen atmosphere. The mixture was allowed to cool down to room temperature. The aqueous layer was extracted with EtOAc (2×50 mL). The resulting mixture was concentrated under reduced pressure. The residue was purified by silica gel column chromatography, eluted with PE/EtOAc (1:2) to afford methyl(S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(2-oxa-6-azaspiro[3.3]heptan-6-yl) propanoyl) hexahydropyridazine-3-carboxylate (6.42 g, 65.03%) as a brown solid. LCMS (ESI): m/z [M+H] calc'd for C₅₇H₈₂N₉O₁₀S 1084.59; found 1084.

### Step 3

To a stirred solution of methyl(S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(2-oxa-6-azaspiro[3.3]heptan-6-yl) propanoyl) hexahydropyridazine-3-carboxylate (6.4 g, 5.90 mmol) in THF (65 mL) and H₂O (65 mL) was added LiOH·H₂O (990.58 mg, 23.61 mmol) in portions at room temperature under an air atmosphere. Desired product could be detected by LCMS. The mixture was acidified to pH 5 with 1N HCl (aq.). The aqueous layer was extracted with CH₂Cl₂ (3×50 mL). The resulting mixture was concentrated under reduced pressure. This resulted in(S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(2-oxa-6-azaspiro[3.3]heptan-6-yl) propanoyl) hexahydropyridazine-3-carboxylic acid (5.7 g, crude) as a brown solid. LCMS (ESI): m/z [M+H] calc'd for C₅₆H₈₀N₉O₁₀S 1070.57; found 1070.50.

### Step 4

To a stirred solution of(S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(2-oxa-6-azaspiro[3.3]heptan-6-yl) propanoyl) hexahydropyridazine-3-carboxylic acid (5.7 g, 5.33 mmol) and DIPEA (27.53 g, 213.00 mmol) in CH₂Cl₂ (570 mL) was added EDCl (24.80 g, 159.75 mmol) and HOBT (7.20 g, 53.25 mmol) in portions at room temperature under an air atmosphere. The resulting mixture was washed with 1N HCl (aq.) (1×100 mL). The resulting mixture was washed with NaHCO₃(aq.) (1×100 mL). The resulting mixture was washed with H₂O (1×100 mL). The organic phase was concentrated under reduced pressure. The residue was purified by silica gel column chromatography, eluted with PE/EtOAc (1:1) to afford tert-butyl ((6³S,3S,4S,Z)-1²-(5-(4-cyclopropylpiperazin-1-yl)-2-((S)-1-methoxyethyl)pyridin-3-yl)-10,10-dimethyl-5,7-dioxo-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (1.7 g, 27.30%) as a brown solid. LCMS (ESI): m/z [M+H] calc'd for C₅₆H₇₈N₉O₉S 1052.56; found 1052.05.

### Step 5

To a stirred solution of tert-butyl ((6³S,3S,4S,Z)-1²-(5-(4-cyclopropylpiperazin-1-yl)-2-((S)-1-methoxyethyl)pyridin-3-yl)-10,10-dimethyl-5,7-dioxo-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (800 mg, 0.76 mmol) in CH₂Cl₂ (9 mL) was added TFA (3 mL, 40.39 mmol) dropwise at 0° C. under an air atmosphere. The reaction mixture was stirred for 1 h at room temperature. The mixture was neutralized to pH 8 with saturated NaHCO₃(aq.). The aqueous layer was extracted with CH₂Cl₂ (2×30 mL). The resulting mixture was concentrated under reduced pressure. This resulted in (6³S,3S,4S,Z)-4-amino-1²-(5-(4-cyclopropylpiperazin-1-yl)-2-((S)-1-methoxyethyl)pyridin-3-yl)-10,10-dimethyl-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (780 mg, crude) as a brown solid. LCMS (ESI): m/z [M+H] calc'd for C₅₁H₇₀N₉O₇S 952.51; found 953.00.

### Step 6

To a stirred solution of (6³S,3S,4S,Z)-4-amino-1²-(5-(4-cyclopropylpiperazin-1-yl)-2-((S)-1-methoxyethyl)pyridin-3-yl)-10,10-dimethyl-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (1.65 g, 1.73 mmol) and (1R,2R,3S)-2,3-dimethylcyclopropane-1-carboxylic acid (257.12 mg, 2.25 mmol) in DMF (17 mL) was added DIPEA (2.24 g, 17.33 mmol) and HATU (988.29 mg, 2.60 mmol) in portions at room temperature. It was stirred for 2 h. Desired product could be detected by LCMS. The resulting mixture was washed with H₂O (3×50 mL). The aqueous layer was extracted with EtOAc (1×50 mL). The resulting mixture was concentrated under reduced pressure. The crude product (1 g) was purified by Prep-HPLC to afford (1r,2R,3S)—N-((6³S,3S,4S,Z)-1²-(5-(4-cyclopropylpiperazin-1-yl)-2-((S)-1-methoxyethyl)pyridin-3-yl)-10,10-dimethyl-5,7-dioxo-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2,3-dimethylcyclopropane-1-carboxamide (890 mg, 48.11%) as a white solid. LCMS (ESI): m/z [M+H] calc'd for C₅₇H₇₈N₉O₈S 1048.57; found 1048.60.

### Intermediate 3. Synthesis of (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile

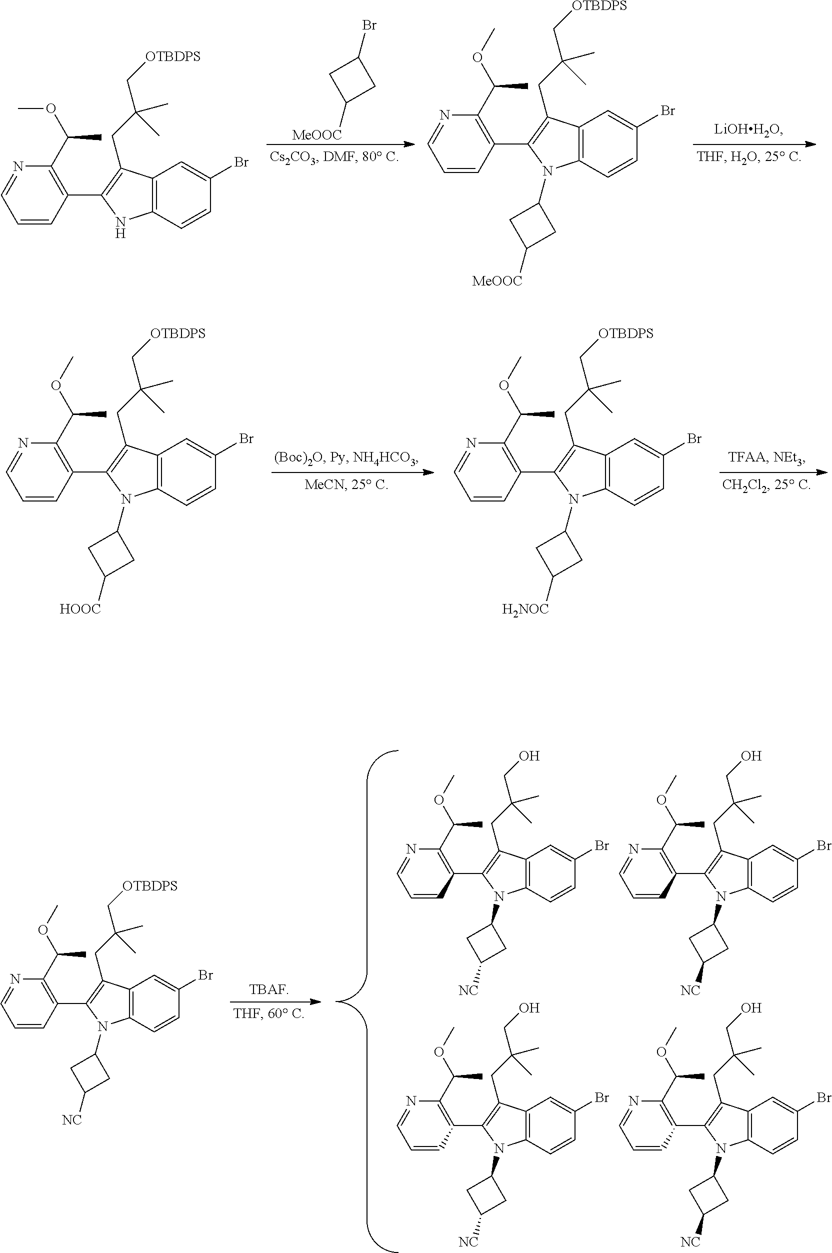

### Step 1

To a stirred solution of(S)-5-bromo-3-(3-((tert-butyldiphenylsilyl)oxy)-2,2-dimethylpropyl)-2-(2-(1-methoxyethyl)pyridin-3-yl)-1H-indole (45 g, 68.62 mmol) and Cs₂CO₃ (44.72 g, 137.25 mmol) in DMF (500 mL) was added methyl 3-bromocyclobutane-1-carboxylate (15.90 g, 82.35 mmol) in portions at 0° C. under an air atmosphere. The resulting mixture was stirred for 16 h at 80° C. The reaction was quenched by the addition of H₂O (500 mL) at 0° C. The resulting mixture was extracted with EtOAc (3×500 mL). The combined organic layers were washed with brine (3×500 mL), dried over anhydrous Na₂SO₄. After filtration, the filtrate was concentrated under reduced pressure. The residue was purified by silica gel column chromatography, eluted with PE/EtOAc (1:1) to afford the product (52 g, 78.95% yield) as a brown yellow solid. LCMS (ESI): m/z [M+H] calc'd for C₄₃H₅₁BrN₂O₄Si 767.28; found 769.30.

### Step 2

To a stirred solution of methyl(S)-3-(5-bromo-3-(3-((tert-butyldiphenylsilyl)oxy)-2,2-dimethylpropyl)-2-(2-(1-methoxyethyl)pyridin-3-yl)-1H-indol-1-yl)cyclobutane-1-carboxylate (52 g, 67.72 mmol) and LIOH·H₂O (5.68 g, 135.44 mmol) in THF (250 mL) and H₂O (250 mL) at 0° C. under an air atmosphere. The resulting mixture was stirred for 2 h at room temperature. The mixture was acidified to pH 6 with citric acid. The resulting mixture was extracted with CH₂Cl₂ (3×500 mL). The combined organic layers were dried over anhydrous Na₂SO₄. After filtration, the filtrate was concentrated under reduced pressure to afford(S)-3-(5-bromo-3-(3-((tert-butyldiphenylsilyl)oxy)-2,2-dimethylpropyl)-2-(2-(1-methoxyethyl)pyridin-3-yl)-1H-indol-1-yl)cyclobutane-1-carboxylic acid (50.5 g, 98.92% yield) as a brown yellow solid. LCMS (ESI): m/z [M+H] calc'd for C₄₂H₄₉BrN₂O₄Si 753.26; found 754.95.

### Step 3

To a stirred solution of(S)-3-(5-bromo-3-(3-((tert-butyldiphenylsilyl)oxy)-2,2-dimethylpropyl)-2-(2-(1-methoxyethyl)pyridin-3-yl)-1H-indol-1-yl)cyclobutane-1-carboxylic acid (50 g, 66.33 mmol) and (Boc)₂O (28.95 g, 132.65 mmol) in MeCN (500 mL) was added NH₄HCO₃ (10.49 g, 132.65 mmol) in portions at 0° C. under air atmosphere. The resulting mixture was stirred for 2 h at room temperature. The reaction was quenched with H₂O/ice at 0° C. The resulting mixture was extracted with EtOAc (3×500 mL). The combined organic layers were dried over anhydrous Na₂SO₄. After filtration, the filtrate was concentrated under reduced pressure. The residue was purified by silica gel column chromatography, eluted with CH₂Cl₂/MeOH (5:1) to afford(S)-3-(5-bromo-3-(3-((tert-butyldiphenylsilyl)oxy)-2,2-dimethylpropyl)-2-(2-(1-methoxyethyl)pyridin-3-yl)-1H-indol-1-yl)cyclobutane-1-carboxamide (47.4 g, 75.94% yield) as a yellow solid. LCMS (ESI): m/z [M+H] calc'd for C₄₂H₅₀BrN₃₀₃Si 752.28; found 754.25.

### Step 4

To a stirred solution of(S)-3-(5-bromo-3-(3-((tert-butyldiphenylsilyl)oxy)-2,2-dimethylpropyl)-2-(2-(1-methoxyethyl)pyridin-3-yl)-1H-indol-1-yl)cyclobutane-1-carboxamide (47 g, 62.43 mmol) and NEts (12.63 g, 124.86 mmol) in CH₂Cl₂ (500 mL) was added TFAA (59.00 g, 280.93 mmol) in portions at 0° C. under air atmosphere. The resulting mixture was stirred for 2 h at room temperature. The reaction was quenched with H₂O/ice at 0° C. The resulting mixture was extracted with CH₂Cl₂ (3×500 mL). The combined organic layers were dried over anhydrous Na₂SO₄. After filtration, the filtrate was concentrated under reduced pressure. The residue was purified by silica gel column chromatography, eluted with CH₂Cl₂/MeOH (5:1) to afford(S)-3-(5-bromo-3-(3-((tert-butyldiphenylsilyl)oxy)-2,2-dimethylpropyl)-2-(2-(1-methoxyethyl)pyridin-3-yl)-1H-indol-1-yl)cyclobutane-1-carbonitrile (41 g, 71.50% yield) as a brown yellow solid. LCMS (ESI): m/z [M+H] calc'd for C₄₂H₄₈BrN₃O₂Si 734.27; found 736.30.

### Step 5

To a stirred solution of(S)-3-(5-bromo-3-(3-((tert-butyldiphenylsilyl)oxy)-2,2-dimethylpropyl)-2-(2-(1-methoxyethyl)pyridin-3-yl)-1H-indol-1-yl)cyclobutane-1-carbonitrile (41 g, 55.79 mmol) and TBAF (87.53 g, 334.76 mmol) in THF (400 mL) at 0° C. under nitrogen atmosphere. The resulting mixture was stirred for 2 h at 60° C. The reaction was quenched by the addition of H₂O/ice (500 mL) at 0° C. The resulting mixture was extracted with EtOAc (3×500 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile A (5.3 g), a mixture of (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile B+ (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile C (15.7 g) and (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile D (5.7 g) as a brown yellow solid. Then, the mixture of (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile B and (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile (15.7 g) was purified by Prep HPLC (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile B (9.7 g) and (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile (2.1 g) as a brown yellow solid.

### Example 2: Synthesis of (1r,2R,3S)—N-((6³S,3S,4S,Z)-1¹-((1r,3S)-3-cyanocyclobutyl)-1²-(2-((S)-1-methoxyethyl)pyridin-3-yl)-10,10-dimethyl-5,7-dioxo-3-(2-oxa-6-azaspiro[3.3]heptan-6-yl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl)-2,3-dimethylcyclopropane-1-carboxamide

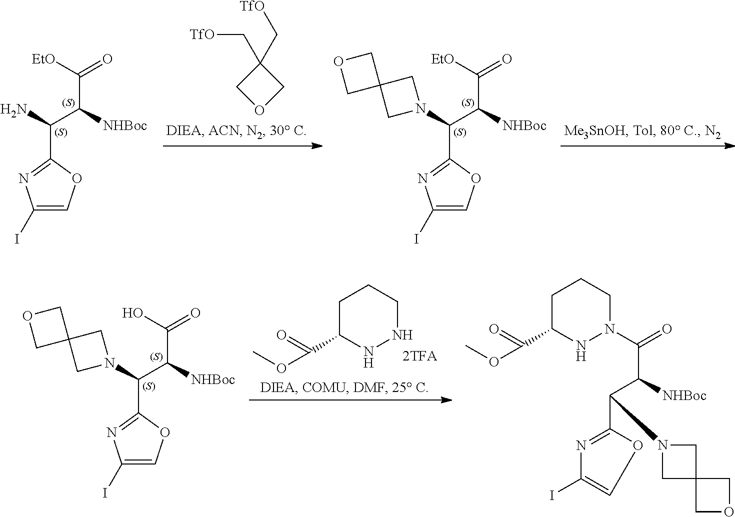

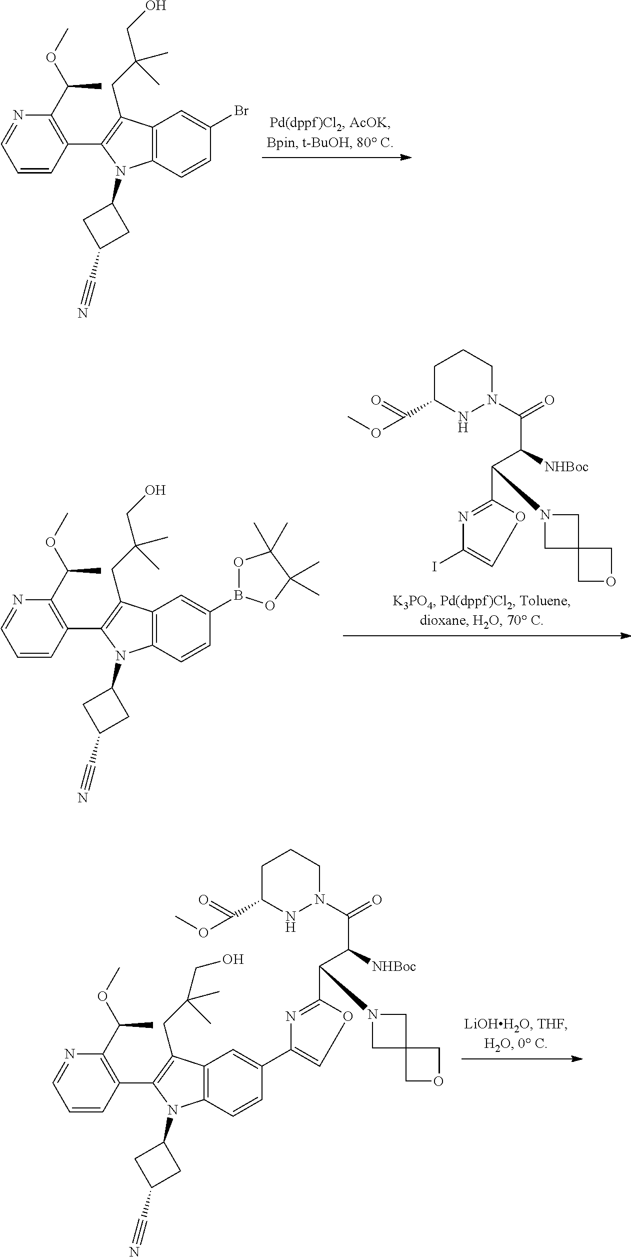 [SMILES: CO[C@@H](C)c1ncccc1-c1c(CC(C)(C)CO)c2cc(Br)ccc2n1[C@H]1C[C@H](C#N)C1 ; CO[C@@H](C)c1ncccc1-c1c(CC(C)(C)CO)c2cc(B3OC(C)(C)C(C)(C)O3)ccc2n1[C@H]1C[C@H](C#N)C1 ; COC(=O)[C@@H]1CCCN(C(=O)[C@@H](NC(=O)OC(C)(C)C)[C@@H](c2nc(I)co2)N2CC3(COC3)C2)N1 ; COC(=O)[C@@H]1CCCN(C(=O)[C@@H](NC(=O)OC(C)(C)C)[C@@H](c2nc(-c3ccc4c(c3)c(CC(C)(C)CO)c(-c3cccnc3[C@H](C)OC)n4[C@H]3C[C@H](C#N)C3)co2)N2CC3(COC3)C2)N1]

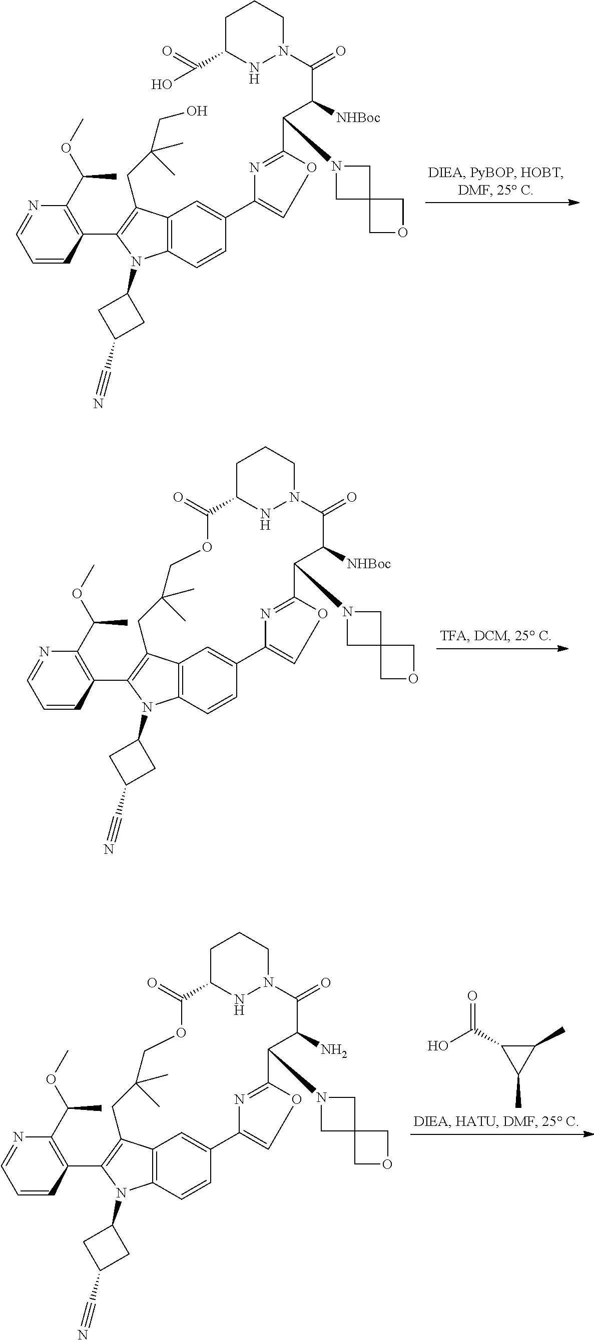

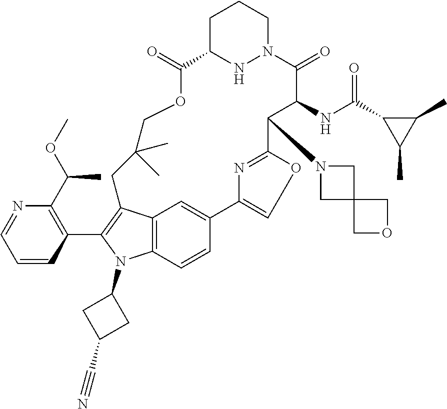

### Step 1

To a stirred solution of ethyl (2S,3S)-3-amino-2-((tert-butoxycarbonyl)amino)-3-(4-iodooxazol-2-yl) propanoate (100 mg, 0.24 mmol) in MeCN (45 mL) was added DIPEA (13.677 g, 105.83 mmol) and oxetane-3,3-diylbis(methylene) bis(trifluoromethanesulfonate) (12.13 g, 31.75 mmol) in portions at 0° C. under a nitrogen atmosphere. The resulting mixture was stirred for additional 2 h at 30° C. The reaction was quenched with H₂O at 0° C. The resulting mixture was extracted with EtOAc (3×100 mL), dried over anhydrous Na₂SO₄. After filtration, the filtrate was concentrated under reduced pressure. The residue was purified by silica gel column chromatography, eluted with PE/EtOAc (1:3) to afford ethyl (2S,3S)-2-((tert-butoxycarbonyl)amino)-3-(4-iodooxazol-2-yl)-3-(2-oxa-6-azaspiro[3.3]heptan-6-yl) propanoate (4.2 g, 70.41% yield) as a white solid. LCMS (ESI): m/z [M+H] calc'd for C₁₈H₂₆IN₃O₆ 508.09; found 508.10.

### Step 2

To a stirred solution of ethyl (2S,3S)-2-((tert-butoxycarbonyl)amino)-3-(4-iodooxazol-2-yl)-3-(2-oxa-6-azaspiro[3.3]heptan-6-yl) propanoate (12 mg, 0.02 mmol) in toluene (42 mL) was added trimethyltin hydroxide (7.48 g, 41.40 mmol) in portions at 20° C. under a nitrogen atmosphere. The resulting mixture was stirred for additional 16 h at 80° C. Desired product could be detected by LCMS. The resulting mixture was filtered, the filter cake was washed with EtOAc (5×50 mL). The combined organic layers were washed with water (3×30 mL), dried over anhydrous Na₂SO₄. After filtration, the filtrate was concentrated under reduced pressure. This resulted in (2S,3S)-2-((tert-butoxycarbonyl)amino)-3-(4-iodooxazol-2-yl)-3-(2-oxa-6-azaspiro[3.3]heptan-6-yl) propanoic acid (3.4 g, 85.69% yield) as a yellow solid. LCMS (ESI): m/z [M+H] calc'd for C₁₆H₂₂IN₃O₆ 480.06; found 480.08.

### Step 3

To a stirred mixture of (2S,3S)-2-((tert-butoxycarbonyl)amino)-3-(4-iodooxazol-2-yl)-3-(2-oxa-6-azaspiro[3.3]heptan-6-yl) propanoic acid (2.9 g, 6.05 mmol) in DMF (29 mL) was added methyl (3S)-1,2-diazinane-3-carboxylate (4.51 mg, 0.03 mmol), DIPEA (7.82 g, 60.51 mmol) and COMU (3.89 g, 9.08 mmol) in portions at 0° C. under an air atmosphere. The resulting mixture was stirred for additional 1 h at 25° C. The reaction was monitored by LCMS. The reaction was quenched by the addition of H₂O (30 mL) at 0° C. The resulting mixture was extracted with EtOAc (3×50 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (1:10) to afford methyl(S)-1-((2S,3S)-2-((tert-butoxycarbonyl)amino)-3-(4-iodooxazol-2-yl)-3-(2-oxa-6-azaspiro[3.3]heptan-6-yl) propanoyl) hexahydropyridazine-3-carboxylate (1.9 g, 40.97% yield) as a yellow solid. LCMS (ESI): m/z [M+H] calc'd for C₂₂H₃₂IN₅O₇ 606.13; found 606.15.

### Step 4

To a solution of (1R,3s)-3-(5-bromo-3-(3-hydroxy-2,2-dimethylpropyl)-2-(2-((S)-1-methoxyethyl)pyridin-3-yl)-1H-indol-1-yl)cyclobutane-1-carbonitrile A (4 g, 8.06 mmol) in t-BuOH (40 mL) was treated with bis(pinacolato)diboron (6.14 g, 24.17 mmol) and KOAc (1.58 g, 16.11 mmol) and Pd(dppf)Cl₂ (1.18 g, 1.611 mmol, 0.2 equiv) for 5 min at 0° C. under nitrogen atmosphere. The resulting mixture was stirred for 2 h at 80° C. under a nitrogen atmosphere. The resulting mixture was concentrated under reduced pressure. The residue was purified by silica gel column chromatography, eluted with PE/EtOAc (1:1) to afford (1 S,3r)-3-(3-(3-hydroxy-2,2-dimethylpropyl)-2-(2-((S)-1-methoxyethyl)pyridin-3-yl)-5-(4,4,5,5-tetramethyl-1,3,2-dioxaborolan-2-yl)-1H-indol-1-yl)cyclobutane-1-carbonitrile (3.8 g, 78.10% yield) as a yellow solid. LCMS (ESI): m/z [M+H] calc'd for C₃₂H₄₂BN₃O₄ 544.33; found 544.35.

### Step 5

To a stirred solution of (1S,3r)-3-(3-(3-hydroxy-2,2-dimethylpropyl)-2-(2-((S)-1-methoxyethyl)pyridin-3-yl)-5-(4,4,5,5-tetramethyl-1,3,2-dioxaborolan-2-yl)-1H-indol-1-yl)cyclobutane-1-carbonitrile (802.01 mg, 1.33 mmol) in toluene (6 mL) and dioxane (2 mL) and H₂O (2 mL) was added K₃PO₄ (585.81 mg, 2.76 mmol) and Pd(dtbpf)Cl₂ (0.1 mg) in portions at 0° C. under a nitrogen atmosphere. The resulting mixture was stirred for 2 h at 70° C. The reaction was quenched by the addition of water/ice (10 mL) at 0° C. The resulting mixture was extracted with EtOAc (3×15 mL). The combined organic layers were dried over anhydrous Na₂SO₄. After filtration, the filtrate was concentrated under reduced pressure. The residue was purified by silica gel column chromatography, eluted with PE/EtOAc (1:1) to afford methyl(S)-1-((2S,3S)-2-((tert-butoxycarbonyl)amino)-3-(4-(1-((1r,3S)-3-cyanocyclobutyl)-3-(3-hydroxy-2,2-dimethylpropyl)-2-(2-((S)-1-methoxyethyl)pyridin-3-yl)-1H-indol-5-yl) oxazol-2-yl)-3-(2-oxa-6-azaspiro[3.3]heptan-6-yl) propanoyl) hexahydropyridazine-3-carboxylate (800 mg, 64.77% yield) as a yellow solid. LCMS (ESI): m/z [M+H] calc'd for C₄₈H₆₂N₈O₉ 895.46; found 895.40.

### Step 6

To a stirred solution of methyl(S)-1-((2S,3S)-2-((tert-butoxycarbonyl)amino)-3-(4-(1-((1,3S)-3-cyanocyclobutyl)-3-(3-hydroxy-2,2-dimethylpropyl)-2-(2-((S)-1-methoxyethyl)pyridin-3-yl)-1H-indol-5-yl) oxazol-2-yl)-3-(2-oxa-6-azaspiro[3.3]heptan-6-yl) propanoyl) hexahydropyridazine-3-carboxylate (800 mg, 0.89 mmol) and LiOH·H₂O (45.00 mg, 1.07 mmol) in THF (5 mL) and H₂O (5 mL) at 0° C. under an air atmosphere. The resulting mixture was stirred for 1 h at 0° C. The mixture was acidified to pH 6 with citric acid. The resulting mixture was extracted with CH₂Cl₂ (3×15 mL). The combined organic layers were dried over anhydrous Na₂SO₄. After filtration, the filtrate was concentrated under reduced pressure to afford(S)-1-((2S,3S)-2-((tert-butoxycarbonyl)amino)-3-(4-(1-((1r,3S)-3-cyanocyclobutyl)-3-(3-hydroxy-2,2-dimethylpropyl)-2-(2-((S)-1-methoxyethyl)pyridin-3-yl)-1H-indol-5-yl) oxazol-2-yl)-3-(2-oxa-6-azaspiro[3.3]heptan-6-yl) propanoyl) hexahydropyridazine-3-carboxylic acid (890 mg, crude) as a yellow solid. LCMS (ESI): m/z [M+H] calc'd for C₄₇H₆₀N₈O₉ 881.45; found 881.35.

### Step 7

To a stirred solution of(S)-1-((2S,3S)-2-((tert-butoxycarbonyl)amino)-3-(4-(1-((1r,3S)-3-cyanocyclobutyl)-3-(3-hydroxy-2,2-dimethylpropyl)-2-(2-((S)-1-methoxyethyl)pyridin-3-yl)-1H-indol-5-yl) oxazol-2-yl)-3-(2-oxa-6-azaspiro[3.3]heptan-6-yl) propanoyl) hexahydropyridazine-3-carboxylic acid (880 mg, 1.0 mmol) and DIPEA (3872.82 mg, 29.97 mmol) in DMF (80 mL) was added PYBOP (2598.92 mg, 5.0 mmol) and HOBT (674.83 mg, 5.0 mmol) in portions at 0° C. under air atmosphere. The resulting mixture was stirred for 2 h at room temperature under air atmosphere. The reaction was quenched by the addition of water/ice (50 mL) at 0° C. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by Prep-HPLC to afford tert-butyl ((6³S,3S,4S,Z)-1¹-((1r,3S)-3-cyanocyclobutyl)-1²-(2-((S)-1-methoxyethyl)pyridin-3-yl)-10,10-dimethyl-5,7-dioxo-3-(2-oxa-6-azaspiro[3.3]heptan-6-yl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl) carbamate (262 mg, 27.35% yield) as a yellow solid. LCMS (ESI): m/z [M+H] calc'd for C₄₇H₅₈N₈O₈ 863.44; found 863.45.

### Step 8

A stirred solution of tert-butyl ((6³S,3S,4S,Z)-1¹-((1r,3S)-3-cyanocyclobutyl)-1²-(2-((S)-1-methoxyethyl)pyridin-3-yl)-10,10-dimethyl-5,7-dioxo-3-(2-oxa-6-azaspiro[3.3]heptan-6-yl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl) carbamate (260 mg, 0.30 mmol) in TFA (1 mL) and CH₂Cl₂ (5 mL) at 0° C. was stirred for 1 h at room temperature under an air atmosphere. The mixture was basified to pH 7 with saturated NaHCO₃(aq.). The resulting mixture was extracted with CH₂Cl₂ (3×5 mL). The combined organic layers were dried over anhydrous Na₂SO₄. After filtration, the filtrate was concentrated under reduced pressure to afford (1S,3r)-3-((6³S,3S,4S,Z)-4-amino-1²-(2-((S)-1-methoxyethyl)pyridin-3-yl)-10,10-dimethyl-5,7-dioxo-3-(2-oxa-6-azaspiro[3.3]heptan-6-yl)-6¹,6²,6³,6⁴,6⁵,6⁶-hexahydro-1¹H-8-oxa-2(4,2)-oxazola-1(5,3)-indola-6(1,3)- pyridazinacycloundecaphane-11-yl)cyclobutane-1-carbonitrile (267 mg, crude) as a yellow green solid. LCMS (ESI): m/z [M+H] calc'd for C₄₂H₅₀N₈O₆ 763.39; found 763.30.

### Step 9

To a stirred solution of (1S,3r)-3-((6³S,3S,4S,Z)-4-amino-1²-(2-((S)-1-methoxyethyl)pyridin-3-yl)-10,10-dimethyl-5,7-dioxo-3-(2-oxa-6-azaspiro[3.3]heptan-6-yl)-6¹,6²,6³,6⁴,6⁵,6⁶-hexahydro-1¹H-8-oxa-2(4,2)-oxazola-1(5,3)-indola-6(1,3)-pyridazinacycloundecaphane-11-yl)cyclobutane-1-carbonitrile (130 mg, 0.17 mmol) and DIPEA (220.24 mg, 1.70 mmol) and (1R,2R,3S)-2,3-dimethylcyclopropane-1-carboxylic acid (38.90 mg, 0.34 mmol) in DMF (3 mL) at 0° C. under an air atmosphere followed by the addition of HATU (129.58 mg, 0.340 mmol, 2 equiv) in portions at 0° C. The resulting mixture was stirred for 1 h at room temperature under air atmosphere. The reaction was quenched with water at 0° C. The resulting mixture was extracted with EtOAc (3×5 mL). The combined organic layers were washed with brine (3×10 mL), dried over anhydrous Na₂SO₄. After filtration, the filtrate was concentrated under reduced pressure. The residue was purified by reversed-phase Prep-HPLC flash to afford (1r,2R,3S)—N-((6³S,3S,4S,Z)-1¹-((1r,3S)-3-cyanocyclobutyl)-1²-(2-((S)-1-methoxyethyl)pyridin-3-yl)-10,10-dimethyl-5,7-dioxo-3-(2-oxa-6-azaspiro[3.3]heptan-6-yl)-6¹,6²,6³,6⁴,6⁵,6⁶-hexahydro-1¹H-8-oxa-2(4,2)-oxazola-1(5,3)- indola-6(1,3)-pyridazinacycloundecaphane-4-yl)-2,3-dimethylcyclopropane-1-carboxamide (28.2 mg, 18.90% yield) as a white solid. LCMS (ESI): m/z [M+H] calc'd for C₄₈H₅₈N₈O₇ 859.44; found 859.45. ¹H NMR (400 MHZ, DMSO-d6) δ 8.44-8.37 (m, 1H), 8.24 (s, 1H), 8.01-8.20 (m, 1H), 7.84-7.77 (m, 1H), 7.75 (s, 1H), 7.56-7.43 (m, 1H), 7.25 (d, J=14.2 Hz, 1H), 7.13 (d, J=6.3 Hz, 1H), 6.98 (s, 1H), 6.81-6.44 (m, 2H), 6.23-6.09 (m, 1H), 5.75-5.62 (m, 1H), 5.43-5.33 (m, 1H), 5.05 (dd, J=16.4, 11.2 Hz, 1H), 4.25 (d, J=12.1 Hz, 1H), 4.04-3.90 (m, 3H), 3.83-3.75 (m, 2H), 3.72-3.60 (m, 2H), 3.65-3.57 (m, 1H), 3.56-3.49 (m, 1H), 3.39-3.29 (m, 3H), 3.08 (d, J=1.7 Hz, 4H), 2.98-2.88 (m, 3H), 2.84 (s, 2H), 2.83-2.69 (m, 3H), 2.63-2.52 (m, 2H), 2.39-2.15 (m, 1H), 2.12 (s, 4H), 2.04 (s, 1H), 1.82 (d, J=12.4 Hz, 1H), 1.68 (s, 1H), 1.53 (t, J=11.8 Hz, 1H), 1.22 (t, J=7.1 Hz, 1H), 0.98 (d, J=6.4 Hz, 2H), 0.89 (d, J=8.4 Hz, 2H), 0.84-0.72 (m, 2H), 0.45 (s, 1H)

### Example 3: Synthesis of (1r,2R,3S)—N-((6³S,3S,4S,Z)-1¹-(1-cyclopropylazetidin-3-yl)-3-(3-methoxyazetidin-1-yl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2,3-dimethylcyclopropane-1-carboxamide

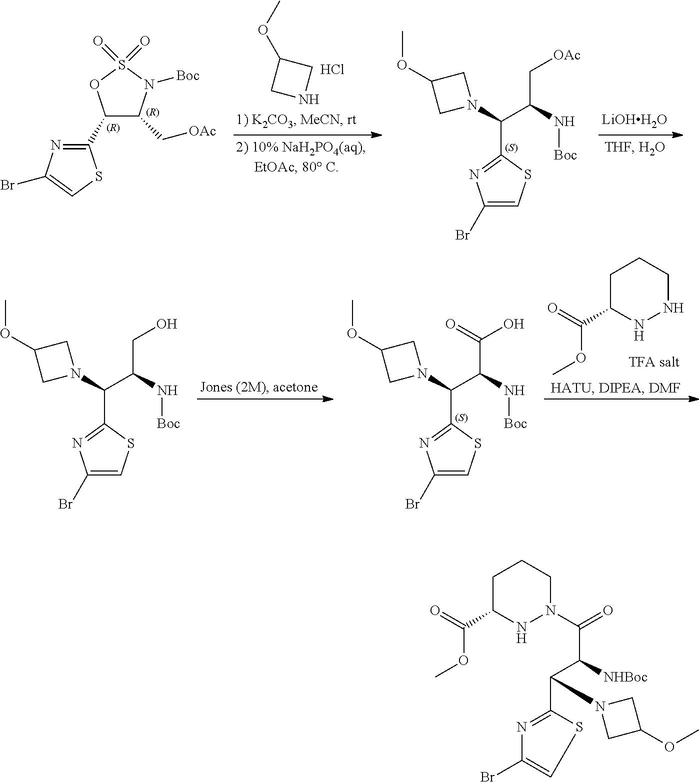

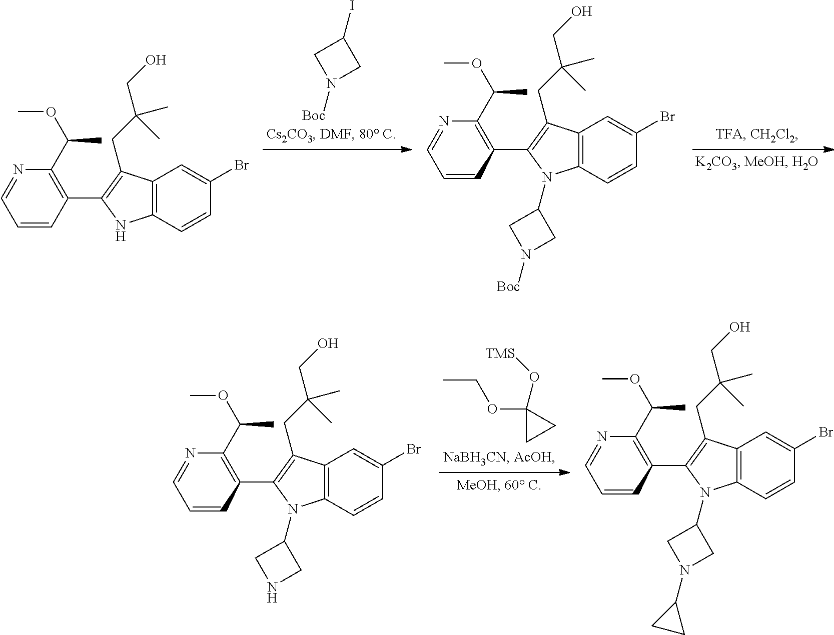

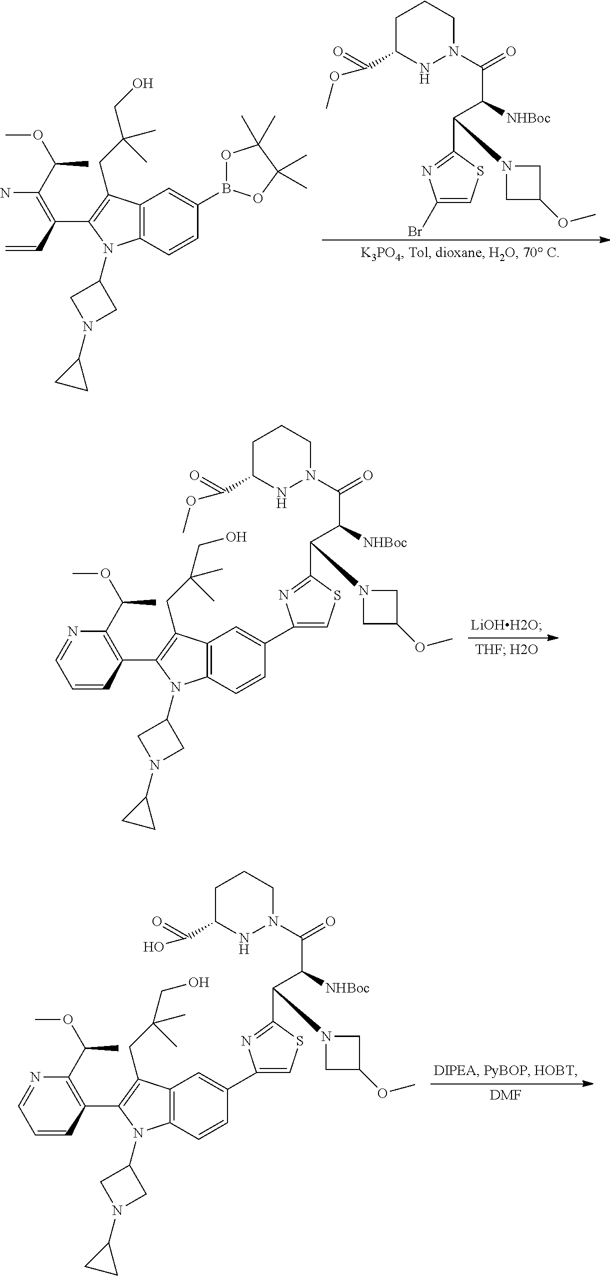

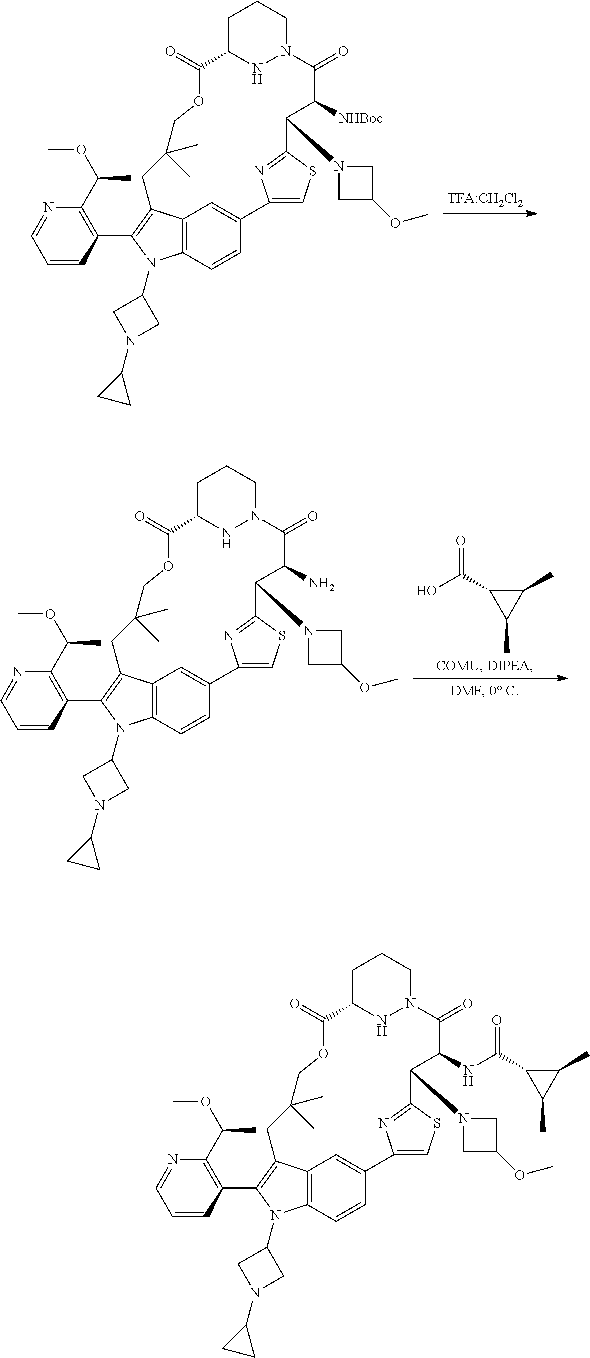

### Step 1

To a stirred solution of 3-methoxyazetidine (34.29 g, 393.61 mmol) and K₂CO₃ (45.33 g, 328.01 mmol) in was added MeCN (300 mL) dropwise at 25° C. The resulting mixture was stirred for 4 h at 25° C. The resulting mixture was filtered, the filter cake was washed with EA (3×100 mL). The filtrate was concentrated under reduced pressure. The residue was dissolved in EA (150 mL). To the above mixture was added tert-butyl (4R,5R)-4-(acetoxymethyl)-5-(4-bromothiazol-2-yl)-1,2,3-oxathiazolidine-3-carboxylate 2,2-dioxide (30 g, 65.60 mmol) and 10% NAH₂PO₄ (150 mL) dropwise over 16 h at 25° C. The resulting mixture was stirred for additional 16 h t at 50° C. Desired product could be detected by LCMS. The resulting mixture was extracted with EtOAc (3×100 mL), dried over anhydrous Na₂SO₄. After filtration, the filtrate was concentrated under reduced pressure to afford (2S,3S)-3-(4-bromothiazol-2-yl)-2-((tert-butoxycarbonyl)amino)-3-(3-methoxyazetidin-1-yl) propyl acetate (28.6 g, crude) as a yellow oil. LCMS (ESI): m/z [M+H] calc'd for C₁₇H₂₆BrN₃O₅S 463.08; found 444.15.

### Step 2

To a stirred solution of (2S,3S)-3-(4-bromothiazol-2-yl)-2-((tert-butoxycarbonyl)amino)-3-(3-methoxyazetidin-1-yl) propyl acetate (29.5 g, 63.53 mmol) in THF (150 mL) were added LiOH·H₂O (10.66 g, 254.10 mmol) and H₂O (150 mL) dropwise at 0° C. The resulting mixture was stirred for 1 h at 25° C. Desired product could be detected by LCMS. The reaction was quenched with H₂O (100 mL) at 0° C. The resulting mixture was extracted with EtOAc (3×200 mL), dried over anhydrous Na₂SO₄. After filtration, the filtrate was concentrated under reduced pressure. This resulted in tert-butyl ((1S,2S)-1-(4-bromothiazol-2-yl)-3-hydroxy-1-(3-methoxyazetidin-1-yl) propan-2-yl) carbamate (21 g, crude) as a yellow solid. ESI-MS m/z=422.00 [M+H]⁺; Calculated MW: 421.07. LCMS (ESI): m/z [M+H] calc'd for C₁₅H₂₄BrN₃O₄S 422.07; found 422.00.

### Step 3

To a stirred solution of tert-butyl ((1 S,2S)-1-(4-bromothiazol-2-yl)-3-hydroxy-1-(3-methoxyazetidin-1-yl) propan-2-yl) carbamate (16.8 g, 16.10 mmol) in acetone (300 mL) was added sulfuric acid; trioxochromium (74.58 mL, 149.17 mmol) in portions at 0° C. The resulting mixture was stirred for 4 h at 25° C. Desired product could be detected by LCMS. The residue was purified by Prep-HPLC to afford (2S,3S)-3-(4-bromo-1,3-thiazol-2-yl)-2-[(tert-butoxycarbonyl)amino]-3-(3-methoxyazetidin-1-yl) propanoic acid (11 g, 50.70% yield) as a white solid. LCMS (ESI): m/z [M+H] calc'd for C₁₅H₂₂BrN₃O₅S 436.05; found 436.00.

### Step 4

To a stirred solution of methyl(S)-hexahydropyridazine-3-carboxylate (12.12 g, 50.42 mmol) and DIPEA (22.36 mL, 128.34 mmol) in DMF (80 mL) were added (2S,3S)-3-(4-bromo-1,3-thiazol-2-yl)-2-[(tert-butoxycarbonyl)amino]-3-(3-methoxyazetidin-1-yl) propanoic acid (11 g, 25.21 mmol) and HATU (10.46 g, 27.50 mmol) dropwise at 0° C. The resulting mixture was stirred for 1 h at 25° C. Desired product could be detected by LCMS. The reaction was quenched by the addition of H₂O (100 mL) at 0° C. The resulting mixture was extracted with EtOAc (3×300 mL). The combined organic layers were washed with brine (3×300 mL), dried over anhydrous Na₂SO₄. After filtration, the filtrate was concentrated under reduced pressure. The residue was purified by silica gel column chromatography, eluted with PE/EtOAc (1:2) to afford methyl(S)-1-((2S,3S)-3-(4-bromothiazol-2-yl)-2-((tert-butoxycarbonyl)amino)-3-(3-methoxyazetidin-1-yl) propanoyl) hexahydropyridazine-3-carboxylate (8.4 g, 59.24% yield) as a yellow solid. LCMS (ESI): m/z [M+H] calc'd for C₂₁H₃₂BrN₅O₆S 562.13; found 562.15.

### Step 5

To a stirred mixture of(S)-3-(5-bromo-2-(2-(1-methoxyethyl)pyridin-3-yl)-1H-indol-3-yl)-2,2-dimethylpropan-1-ol (50 g, 119.80 mmol) and Cs₂CO₃ (195.17 g, 599.02 mmol) in DMF (500 mL) was added tert-butyl 3-iodoazetidine-1-carboxylate (169.59 g, 599.02 mmol) dropwise at 70° C. under an air atmosphere. The resulting mixture was stirred for 16 h at 80° C. The resulting mixture was filtered, the filter cake was washed with EtOAc (3×500 mL). The combined organic layers were washed with brine (3×800 mL), dried over anhydrous Na₂SO₄. After filtration, the filtrate was concentrated under reduced pressure. The residue was purified by silica gel column chromatography, eluted with PE/EtOAc (1:1) to afford tert-butyl(S)-3-(5-bromo-3-(3-hydroxy-2,2-dimethylpropyl)-2-(2-(1-methoxyethyl)pyridin-3-yl)-1H-indol-1-yl) azetidine-1-carboxylate (37.3 g, 48.94% yield) as a yellow solid. LCMS (ESI): m/z [M+H] calc'd for C₂₉H₃₈BrN₃O₄ 572.20; found 572.2.

### Step 6

To a stirred solution of tert-butyl(S)-3-(5-bromo-3-(3-hydroxy-2,2-dimethylpropyl)-2-(2-(1-methoxyethyl)pyridin-3-yl)-1H-indol-1-yl) azetidine-1-carboxylate (37.3 g, 65.15 mmol) in CH₂Cl₂ (270 mL) was added TFA (90 mL) dropwise at 0° C. under an air atmosphere. The resulting mixture was stirred for 3 h at 25° C. The resulting mixture was concentrated under reduced pressure. The residue was dissolved in MeOH (180 mL). To the above mixture was added K₂CO₃ (90.04 g, 651.48 mmol) in H₂O (180 mL) dropwise over 10 min at 0° C. The resulting mixture was stirred for additional 5 h at 25° C. The resulting mixture was concentrated under reduced pressure. The resulting mixture was extracted with CH₂Cl₂ (3×500 mL). The combined organic layers were dried over anhydrous Na₂SO₄. After filtration, the filtrate was concentrated under reduced pressure. The crude product(S)-3-(1-(azetidin-3-yl)-5-bromo-2-(2-(1-methoxyethyl)pyridin-3-yl)-1H-indol-3-yl)-2,2-dimethylpropan-1-ol (31.3 g, 86.44%) was used in the next step directly without further purification. LCMS (ESI): m/z [M+H] calc'd for C₂₄H₃₀BrN₃O₂ 472.15; found 472.2.

### Step 7

To a stirred mixture of(S)-3-(1-(azetidin-3-yl)-5-bromo-2-(2-(1-methoxyethyl)pyridin-3-yl)-1H-indol-3-yl)-2,2-dimethylpropan-1-ol (30.2 g, 63.93 mmol) and (1-ethoxycyclopropoxy)trimethylsilane (33.43 g, 191.78 mmol) in MeOH (450 mL) were added AcOH (10.99 mL) and NaBH₃CN (8.03 g, 127.85 mmol) in portions at 0° C. under an air atmosphere. The resulting mixture was stirred for 4 h at 60° C. under a nitrogen atmosphere. The reaction was quenched with H₂O/Ice at 0° C. The mixture was basified to pH 8 with saturated NaHCO₃(aq.). The resulting mixture was concentrated under reduced pressure. Then resulting mixture was extracted with CH₂Cl₂ (3×500 mL). The combined organic layers were washed with saturated NaHCO₃(aq.) (3×200 mL), dried over anhydrous Na₂SO₄. After filtration, the filtrate was concentrated under reduced pressure. The residue was purified by trituration with MTBE (120 mL) to afford(S)-3-(5-bromo-1-(1-cyclopropylazetidin-3-yl)-2-(2-(1-methoxyethyl)pyridin-3-yl)-1H-indol-3-yl)-2,2-dimethylpropan-1-ol (18.3 g, 53.07% yield) as a white solid. LCMS (ESI): m/z [M+H] calc'd for C₂₇H₃₄BrN₃O₂ 512.18; found 512.2.

### Step 8

1,3,2-dioxaborolan-2-yl)-1H-indol-3-yl)-2,2-dimethylpropan-1-ol (361.88 mg, 0.64 mmol) in toluene (3 mL) was added K₃PO₄ (227.61 mg, 1.07 mmol), Pd(dtbpf)Cl₂ (69.89 mg, 0.11 mmol), dioxane (1 mL), H₂O (1 mL) in portions at 25° C. under an air atmosphere. The resulting mixture was stirred for 1 h at 70° C. under a nitrogen atmosphere. The reaction was monitored by LCMS. The reaction was quenched with H₂O at 0° C. The resulting mixture was extracted with EtOAc (3×20 mL), dried over anhydrous Na₂SO₄. After filtration, the filtrate was concentrated under reduced pressure. The residue was purified by Prep-HPLC to afford methyl(S)-1-((2S,3S)-2-((tert-butoxycarbonyl)amino)-3-(4-(1-(1-cyclopropylazetidin-3-yl)-3-(3-hydroxy-2,2-dimethylpropyl)-2-(2-((S)-1-methoxyethyl)pyridin-3-yl)-1H-indol-5-yl) thiazol-2-yl)-3-(3-methoxyazetidin-1-yl) propanoyl) hexahydropyridazine-3-carboxylate (408 mg, 66.52% yield) as a white solid. LCMS (ESI): m/z [M+H] calc'd for C₄₈H₆₆N₈O₈S 915.47; found 915.50.

### Step 9

To a stirred solution of methyl(S)-1-((2S,3S)-2-((tert-butoxycarbonyl)amino)-3-(4-(1-(1-cyclopropylazetidin-3-yl)-3-(3-hydroxy-2,2-dimethylpropyl)-2-(2-((S)-1-methoxyethyl)pyridin-3-yl)-1H-indol-5-yl) thiazol-2-yl)-3-(3-methoxyazetidin-1-yl) propanoyl) hexahydropyridazine-3-carboxylate (403 mg, 0.44 mmol) in THF (2.5 mL) was added LiOH·H₂O (27.72 mg, 0.66 mmol) in H₂O (2.5 mL) dropwise at 0° C. under an air atmosphere. The resulting mixture was stirred for 1 h at 25° C. under air atmosphere. The reaction was monitored by LCMS. The mixture was acidified to pH 6 with citric acid. The resulting mixture was extracted with CH₂Cl₂ (3×20 mL), dried over anhydrous Na₂SO₄. After filtration, the filtrate was concentrated under reduced pressure. This resulted in(S)-1-((2S,3S)-2-((tert-butoxycarbonyl)amino)-3-(4-(1-(1-cyclopropylazetidin-3-yl)-3-(3-hydroxy-2,2-dimethylpropyl)-2-(2-((S)-1-methoxyethyl)pyridin-3-yl)-1H-indol-5-yl) thiazol-2-yl)-3-(3-methoxyazetidin-1-yl) propanoyl) hexahydropyridazine-3-carboxylic acid (450 mg, crude) as a light yellow solid. LCMS (ESI): m/z [M+H] calc'd for C₄₇H₆₄N₈O₈S 901.46; found 901.6.

### Step 10

To a stirred solution of(S)-1-((2S,3S)-2-((tert-butoxycarbonyl)amino)-3-(4-(1-(1-cyclopropylazetidin-3-yl)-3-(3-hydroxy-2,2-dimethylpropyl)-2-(2-((S)-1-methoxyethyl)pyridin-3-yl)-1H-indol-5-yl) thiazol-2-yl)-3-(3-methoxyazetidin-1-yl) propanoyl) hexahydropyridazine-3-carboxylic acid (420 mg, 0.47 mmol) in DMF (42 mL) was added DIPEA (1.81 g, 13.98 mmol), HOBT (629.79 mg, 4.66 mmol) and PyBOP (2.43 g, 4.66 mmol) at 0° C. under an air atmosphere. The resulting mixture was stirred for 5 h at 25° C. The reaction was monitored by LCMS. The reaction was quenched with H₂O at 0° C. The mixture was neutralized to pH 7 with citric acid. The resulting mixture was extracted with EtOAc (3×30 mL). The combined organic layers were washed with brine (3×150 mL), dried over anhydrous Na₂SO₄. After filtration, the filtrate was concentrated under reduced pressure. The residue was purified by Prep-HPLC to afford tert-butyl ((6³S,3S,4S,Z)-1¹-(1-cyclopropylazetidin-3-yl)-3-(3-methoxyazetidin-1-yl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (184 mg, 40.23% yield) as a brown solid. LCMS (ESI): m/z [M+H] calc'd for C₄₇H₆₂N₈O₇S 883.45; found 883.5.

### Step 11

To a stirred solution of tert-butyl ((6³S,3S,4S,Z)-1¹-(1-cyclopropylazetidin-3-yl)-3-(3-methoxyazetidin-1-yl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (80 mg, 0.09 mmol) in CH₂Cl₂ (0.9 mL) was added TFA (0.3 mL) dropwise at 0° C. under an air atmosphere. The resulting mixture was stirred for 1 h at 0° C. The reaction was monitored by LCMS. The mixture was basified to pH 8 with saturated NaHCO₃ (aq.). The resulting mixture was extracted with CH₂Cl₂ (3×15 mL), dried over anhydrous Na₂SO₄. After filtration, the filtrate was concentrated under reduced pressure. The crude product mixture was used in the next step directly without further purification. This resulted in (6³S,3S,4S,Z)-4-amino-1¹-(1-cyclopropylazetidin-3-yl)-3-(3-methoxyazetidin-1-yl)-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (123 mg, crude) as a yellow solid. LCMS (ESI): m/z [M+H] calc'd for C₄₂H₅₄N₈O₅S 783.39; found 783.4.

### Step 12

A mixture of (6³S,3S,4S,Z)-4-amino-1¹-(1-cyclopropylazetidin-3-yl)-3-(3-methoxyazetidin-1-yl)-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (200.00 mg, 0.31 mmol), (2S)-3-methyl-2-[N-methyl-1-[(3S)-1-(prop-2-enoyl) pyrrolidin-3-yl]formamido]butanoic acid (171.95 mg, 0.61 mmol), DIPEA (393.55 mg, 3.05 mmol) and COMU (260.66 mg, 0.61 mmol) in DMF (2.0 ml) was stirred for 30 min at 0° C. under an air atmosphere. The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC to afford (1r,2R,3S)—N-((6³S,3S,4S,Z)-1¹-(1-cyclopropylazetidin-3-yl)-3-(3-methoxyazetidin-1-yl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2,3-dimethylcyclopropane-1-carboxamide (44.6 mg, 33.8% yield,) as a white solid. LCMS (ESI): m/z [M+H] calc'd for C₄₈H₆₂N₈O₆S 879.45; found 879.3. ¹H NMR (400 MHZ, DMSO-d6) δ 8.53-8.46 (m, 1H), 8.28 (s, 1H), 7.81-7.64 (m, 2H), 7.55 (d, J=8.7 Hz, 1H), 7.47-7.33 (m, 2H), 7.21 (m, 1H), 5.44 (d, J=9.2 Hz, 1H), 4.83 (d, J=12.2 Hz, 1H), 4.56-4.44 (m, 1H), 4.09-3.99 (m, 3H), 3.92-3.86 (m, 1H), 3.76-3.74 (m, 1H), 3.45-3.39 (m, 2H), 3.35-3.25 (m, 2H), 3.09-3.01 (m, 1H), 3.00 (s, 3H), 2.89 (s, 5H), 2.71 (s, 2H), 2.56 (d, J=26.7 Hz, 2H), 2.33 (d, J=5.7 Hz, 5H), 2.21 (s, 1H), 1.95-1.83 (m, 1H), 1.74-1.61 (m, 2H), 1.51 (s, 1H), 1.38-1.25 (m, 2H), 1.13 (d, J=6.1 Hz, 3H), 0.98 (d, J=7.5 Hz, 2H), 0.92-0.84 (m, 5H), 0.72 (s, 3H), 0.10 (d, J=6.4 Hz, 4H).

### Intermediate 4. Synthesis of(S)-2-((2S,3S)-3-(2-bromooxazol-4-yl)-2-((tert-butoxycarbonyl)amino)-3-(pyrrolidin-1-yl) propanoyl)-2,3-diazabicyclo[3.1.1]heptane-4-carboxylic acid

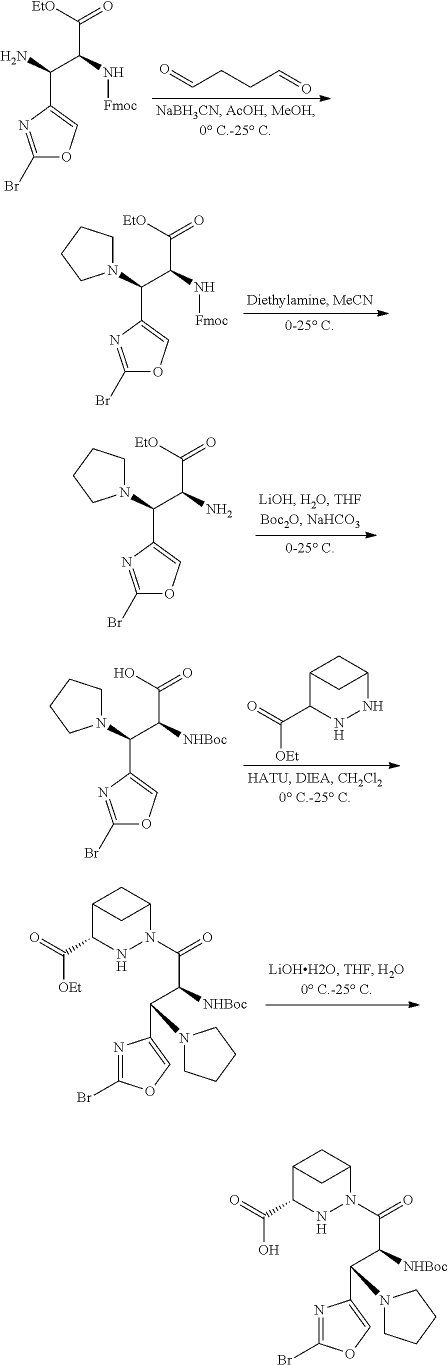

### Step 1

To a stirred mixture of ethyl (2S,3S)-2-((((9H-fluoren-9-yl) methoxy) carbonyl)amino)-3-amino-3-(2-bromooxazol-4-yl) propanoate (7.4 g, 14.79 mmol) and succinaldehyde (3.82 g, 44.37 mmol) in MeOH (80 mL) were added AcOH (2.66 g, 44.37 mmol) dropwise at 0° C. The resulting mixture was stirred for 30 min at 0° C. To the above mixture was added NaBH₃CN (2.79 g, 44.37 mmol) in portions at 0° C. The resulting mixture was stirred for additional 1 h at 25° C. The resulting mixture was diluted with H₂O (100 mL). The resulting mixture was extracted with EtOAc (3×100 mL), dried over anhydrous Na₂SO₄. After filtration, the filtrate was concentrated under reduced pressure. The residue was purified by silica gel column chromatography, eluted with PE/EtOAc (17%) to afford ethyl (2S,3S)-2-((((9H-fluoren-9-yl) methoxy) carbonyl)amino)-3-(2-bromooxazol-4-yl)-3-(pyrrolidin-1-yl) propanoate (8 g, 78% yield) as a yellow solid. LCMS (ESI): m/z [M+H] calc'd for C₂₇H₂₈BrN₃O₅ 554.12; found 554.2.

### Step 2

Into a 500 mL round-bottom flask were added ethyl (2S,3S)-2-((((9H-fluoren-9-yl) methoxy) carbonyl)amino)-3-(2-bromooxazol-4-yl)-3-(pyrrolidin-1-yl) propanoate (7.4 g, 13.35 mmol), MeCN (74 mL) and diethylamine (74 mL) at 0° C. The resulting mixture was stirred for 1 h at 25° C. The resulting mixture was concentrated under reduced pressure to afford ethyl (2S,3S)-2-amino-3-(2-bromooxazol-4-yl)-3-(pyrrolidin-1-yl) propanoate (8 g, crude) as a yellow solid. The crude product was used in the next step directly without further purification. LCMS (ESI): m/z [M+H] calc'd for C₁₂H₁₈BrN₃O₃ 332.05; found 332.2.

### Step 3

To a stirred mixture of ethyl (2S,3S)-2-amino-3-(2-bromooxazol-4-yl)-3-(pyrrolidin-1-yl) propanoate (8 g, 24.08 mmol) in THF (80 mL) and H₂O (80 mL) were added LiOH·H₂O (5.05 g, 120.41 mmol) in portions at 0° C. The resulting mixture was stirred for 10 h at 25° C. Desired product could be detected by LCMS. To the above mixture was added NaHCO₃ (17.68 g, 210.42 mmol) and (Boc)₂O (11.48 g, 52.61 mmol) dropwise at 0° C. The resulting mixture was stirred for additional 2 h at 25° C. The resulting mixture was extracted with EtOAc (2×100 mL), the aqueous layer was neutralized to pH 5 with citric acid. The aqueous layer was extracted with CHCl₃: i-PrOH=3:1 (3×100 mL). After filtration, the filtrate was concentrated under reduced pressure. The residue was purified by Prep-HPLC to afford (2S,3S)-3-(2-bromooxazol-4-yl)-2-((tert-butoxycarbonyl)amino)-3-(pyrrolidin-1-yl) propanoic acid (2.4 g, 23% yield) as a light yellow solid. LCMS (ESI): m/z [M+H] calc'd for C₁₅H₂₂BrN₃O₅ 404.07; found 403.9.

### Step 4

To a stirred mixture of ethyl(S)-2,3-diazabicyclo[3.1.1]heptane-4-carboxylate (551.37 mg, 1.36 mmol) in CH₂Cl₂ (5 mL) were added DIPEA (1598.56 mg, 12.37 mmol), (2S,3S)-3-(2-bromooxazol-4-yl)-2-((tert-butoxycarbonyl)amino)-3-(pyrrolidin-1-yl) propanoic acid (500 mg, 1.24 mmol) and HATU (611.37 mg, 1.61 mmol) in portions at 0° C. The resulting mixture was stirred for 30 min at 25° C. The resulting mixture was diluted with H₂O (20 mL). The resulting mixture was extracted with CH₂Cl₂ (3×20 mL). The combined organic layers were washed with water (2×10 mL), dried over anhydrous Na₂SO₄. After filtration, the filtrate was concentrated under reduced pressure. The residue was purified by silica gel column chromatography, eluted with PE/EtOAc (1:1) to afford ethyl(S)-2-((2S,3S)-3-(2-bromooxazol-4-yl)-2-((tert-butoxycarbonyl)amino)-3-(pyrrolidin-1-yl) propanoyl)-2,3-diazabicyclo[3.1.1]heptane-4-carboxylate (540 mg, 74% yield) as a white solid. LCMS (ESI): m/z [M+H] calc'd for C₂₃H₃₄BrN₅O 556.17; found 556.1.

### Step 5

To a stirred mixture of ethyl(S)-2-((2S,3S)-3-(2-bromooxazol-4-yl)-2-((tert-butoxycarbonyl)amino)-3-(pyrrolidin-1-yl) propanoyl)-2,3-diazabicyclo[3.1.1]heptane-4-carboxylate (540 mg, 0.97 mmol) in THF (5 mL) and H₂O (5 mL) were added LiOH·H₂O (122.16 mg, 2.91 mmol) in portions at 0° C. The resulting mixture was stirred for 1 h at room temperature. Desired product could be detected by LCMS. The mixture was acidified to pH 5 with citric acid. The resulting mixture was extracted with EtOAc (3×20 mL). The combined organic layers were washed with water (2×10 mL), dried over anhydrous Na₂SO₄. After filtration, the filtrate was concentrated under reduced pressure to afford(S)-2-((2S,3S)-3-(2-bromooxazol-4-yl)-2-((tert-butoxycarbonyl)amino)-3-(pyrrolidin-1-yl) propanoyl)-2,3-diazabicyclo[3.1.1]heptane-4-carboxylic acid (500 mg, crude) as a white solid. LCMS (ESI): m/z [M+H] calc'd for C₂₁H₃₀BrN₅O₆ 528.14; found 528.1.

### Intermediate 5. Synthesis of (1R,2S,3R)-2-methyl-3-(pyridin-2-yl) cyclopropane-1-carboxylic acid

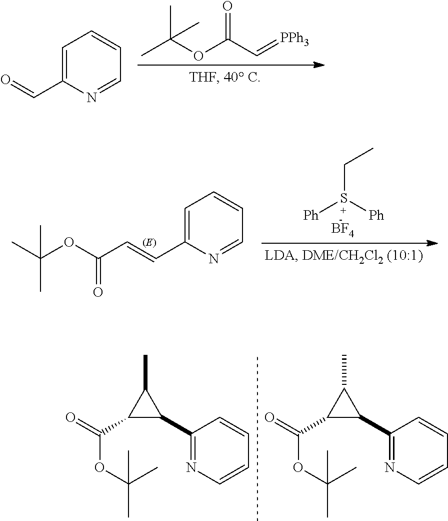

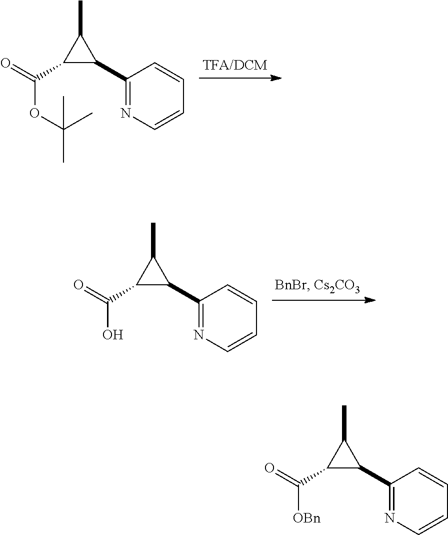

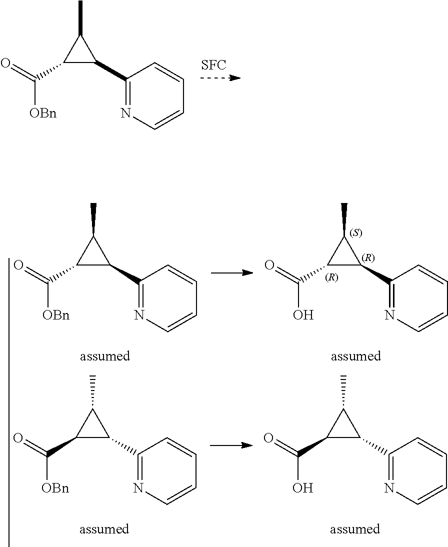

### Step 1

A solution of 2-formylpyridine (20 g, 186.720 mmol) and tert-butyl 2-(triphenyl-lambda5-phosphanylidene)acetate (105.43 g, 280.080 mmol) in THF (200 mL) was stirred for 2 h at 40° C. under air atmosphere. The resulting mixture was concentrated under reduced pressure. The residue was washed with H₂O (2×100 mL). The aqueous layer was extracted with EtOAc (2×100 mL). The residue was purified by silica gel column chromatography, eluted with pet. ether/EtOAc (1:1) to afford tert-butyl (2E)-3-(pyridin-2-yl) prop-2-enoate (21.1 g, 55.05%) as a yellow oil. LCMS (ESI): m/z [M+H] calc'd for C₁₂H₁₅NO₂ 205.11; found 205.11.

### Step 2

A solution of ethyldiphenylsulfanium tetrafluoroborate (64.84 g, 214.605 mmol) and LDA (26.82 g, 250.373 mmol) in DME (570 mL) and CH₂Cl₂ (57 mL) was stirred for 30 min at −78° C. under a nitrogen atmosphere. To the above mixture was added tert-butyl (2E)-3-(pyridin-2-yl) prop-2-enoate (14.683 g, 71.535 mmol) dropwise over 5 min at −78° C. The resulting mixture was stirred for an additional 1 h at room temperature. The reaction was quenched with sat. aq. NH₄Cl at room temperature. The aqueous layer was extracted with CH₂Cl₂ (2×200 mL). The residue was purified by silica gel column chromatography, eluted with pet. ether/EtOAc (1:1) to afford tert-butyl (1S)-2-methyl-3-(pyridin-2-yl) cyclopropane-1-carboxylate (5 g, 29.9% yield) as yellow oil and tert-butyl (1R)-2-methyl-3-(pyridin-2-yl) cyclopropane-1-carboxylate (4.07 g, 24.2% yield). LCMS (ESI): m/z [M+H] calc'd for C₁₄H₁₉NO₂ 233.14; found 233.14.

### Step 3

A solution of tert-butyl (1R)-2-methyl-3-(pyridin-2-yl) cyclopropane-1-carboxylate (10.9 g, 46.719 mmol) and TFA (30 mL, 403.892 mmol) in CH₂Cl₂ (30 mL) was stirred for 1 h at room temperature under air atmosphere. The resulting mixture was concentrated under reduced pressure. The crude resulting mixture was used in the next step directly without further purification. LCMS (ESI): m/z [M+H] calc'd for C₁₀H₁₁NO₂ 177.08; found 177.08.

### Step 4

To a stirred solution of (1R,2S,3R)-2-methyl-3-(pyridin-2-yl) cyclopropane-1-carboxylic acid (9 g, 50.789 mmol) and Cs₂CO₃ (49.64 g, 152.367 mmol) in DMF (100 mL) were added KI (1.69 g, 10.158 mmol) and BnBr (13.03 g, 76.184 mmol) dropwise at room temperature under air atmosphere. The residue was washed with brine (1×50 mL). The aqueous layer was extracted with EtOAc (2×100 mL). The resulting mixture was concentrated under reduced pressure. The residue was purified by silica gel column chromatography, eluted with PE/EtOAc (1:1) to afford benzyl (1R,2S,3R)-2-methyl-3-(pyridin-2-yl) cyclopropane-1-carboxylate (5.9 g, 65.55%) as a yellow oil and mixture of two isomers. The mixture of isomers was separated by chiral-SFC to afford benzyl (1 S,2R,3S)-2-methyl-3-(pyridin-2-yl) cyclopropane-1-carboxylate A (1.18 g, 48.1% yield) as colorless oil and benzyl (1R,2S,3R)-2-methyl-3-(pyridin-2-yl) cyclopropane-1-carboxylate B (1.32 g, 53.8% yield) as colorless oil. LCMS (ESI): m/z [M+H] calc'd for C₁₇H₁₇NO₂ 267.13; found 267.13.

### Step 5

A solution of benzyl (1R,2S,3R)-2-methyl-3-(pyridin-2-yl) cyclopropane-1-carboxylate (2.5 g, 9.352 mmol) and LiOH·H₂O (0.27 g, 11.222 mmol) in MeOH (5 mL) and THF (5 mL) and H₂O (5 mL) was stirred for 1 h at room temperature under air atmosphere. The resulting mixture was concentrated under reduced pressure. The aqueous layer was extracted with 2-methoxy-2-methylpropane (2×20 mL). The aqueous layer was diluted with MeCN (5 mL) to lyophilization. This resulted in (1R,2S,3R)-2-methyl-3-(pyridin-2-yl) cyclopropane-1-carboxylic acid (2.7 g, crude) as a white solid. LCMS (ESI): m/z [M+H] calc'd for C₁₀H₁₁NO₂ 177.1; found 177.1.

### Example 4: Synthesis of (1R,2S,3R)—N-((6⁴S,3S,4S,Z)-1²-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-5,7-dioxo-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-4-yl)-2-methyl-3-(pyridin-2-yl) cyclopropane-1-carboxamide

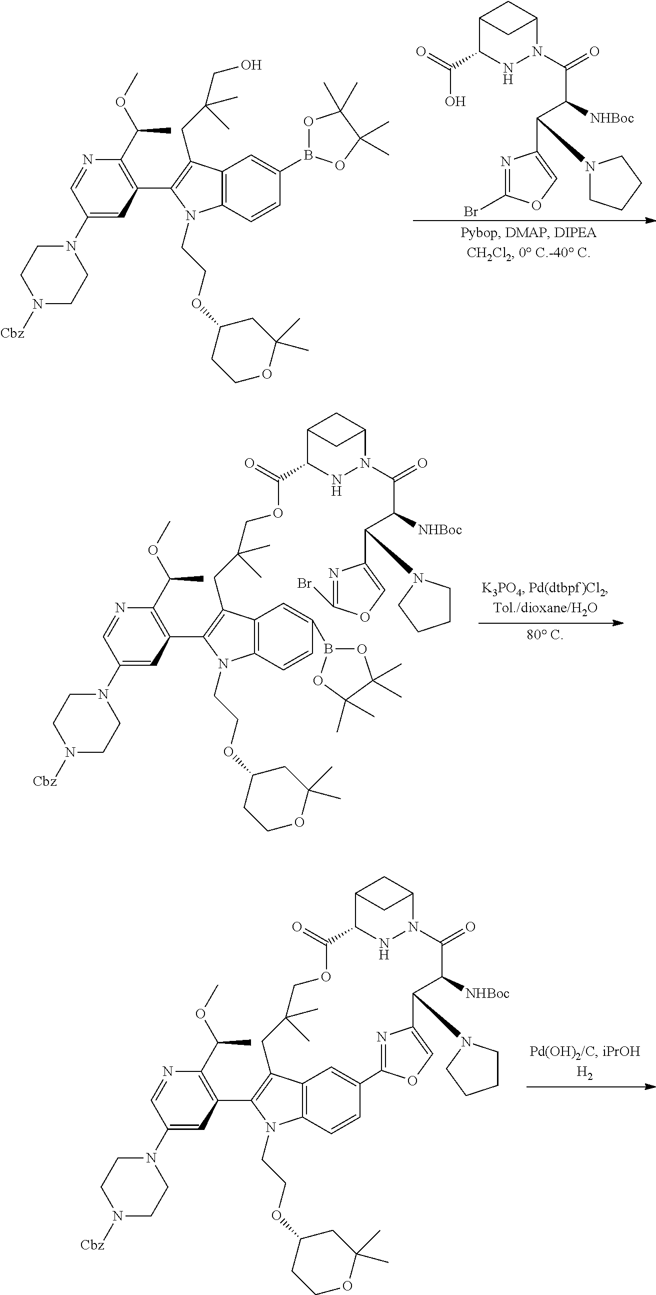

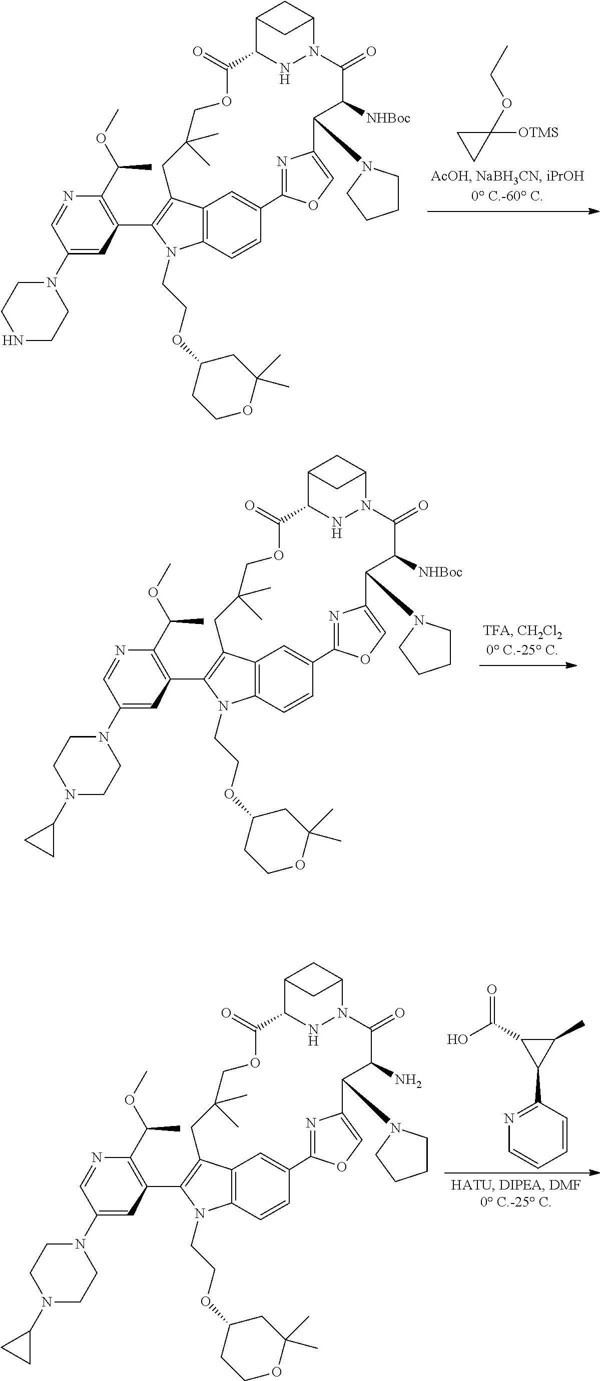

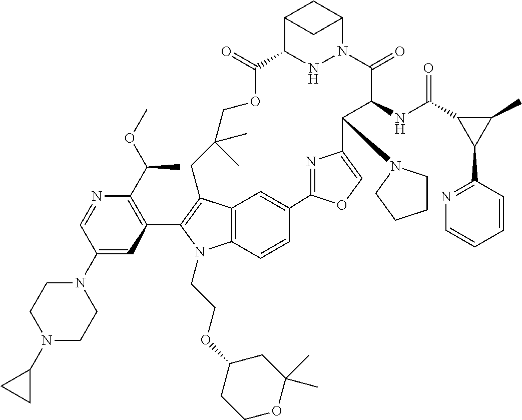

### Step 1

To a stirred solution of benzyl 4-(5-(1-(2-(((S)-2,2-dimethyltetrahydro-2H-pyran-4-yl)oxy)ethyl)-3-(3-hydroxy-2,2-dimethylpropyl)-5-(4,4,5,5-tetramethyl-1,3,2-dioxaborolan-2-yl)-1H-indol-2-yl)-6-((S)-1-methoxyethyl)pyridin-3-yl) piperazine-1-carboxylate (600 mg, 0.715 mmol, 1 equiv) and (S)-2-((2S,3S)-3-(2-bromooxazol-4-yl)-2-((tert-butoxycarbonyl)amino)-3-(pyrrolidin-1-yl) propanoyl)-2,3-diazabicyclo[3.1.1]heptane-4-carboxylic acid (453.52 mg, 0.86 mmol) in CH₂Cl₂ (3 mL) were added DIPEA (924.41 mg, 7.15 mmol), DMAP (131.07 mg, 1.07 mmol) and PyBOP (1.16 g, 2.15 mmol) in portions at 0° C. The resulting mixture was stirred for 1 h at 40° C. The resulting mixture was concentrated under reduced pressure. The residue was purified by silica gel column chromatography, eluted with PE/EtOAc (1:10) to afford 3-(2-(5-(4-((benzyloxy) carbonyl) piperazin-1-yl)-2-((S)-1-methoxyethyl)pyridin-3-yl)-1-(2-(((S)-2,2-dimethyltetrahydro-2H-pyran-4-yl)oxy)ethyl)-5-(4,4,5,5-tetramethyl-1,3,2-dioxaborolan-2-yl)-1H-indol-3-yl)-2,2-dimethylpropyl(S)-2-((2,3S)-3-(2-bromooxazol-4-yl)-2-((tert-butoxycarbonyl)amino)-3-(pyrrolidin-1-yl) propanoyl)-2,3-diazabicyclo[3.1.1]heptane-4-carboxylate (1.5 g, 77% yield) as a brown yellow solid. LCMS (ESI): m/z [M+H] calc'd for C₆₉H₉₅BBrN₉O₁₃ 1348.63; found 1348.4.

### Step 2

To a solution of 3-(2-(5-(4-((benzyloxy) carbonyl) piperazin-1-yl)-2-((S)-1-methoxyethyl)pyridin-3-yl)-1-(2-(((S)-2,2-dimethyltetrahydro-2H-pyran-4-yl)oxy)ethyl)-5-(4,4,5,5-tetramethyl-1,3,2-dioxaborolan-2-yl)-1H-indol-3-yl)-2,2-dimethylpropyl(S)-2-((2S,3S)-3-(2-bromooxazol-4-yl)-2-((tert-butoxycarbonyl)amino)-3-(pyrrolidin-1-yl) propanoyl)-2,3-diazabicyclo[3.1.1]heptane-4-carboxylate (1.5 g, 1.11 mmol) and K₃PO₄ (707.93 mg, 3.34 mmol) in toluene (42 mL), dioxane (14 mL) and H₂O (14 mL) were added Pd(DtBPF)Cl₂ (72.46 mg, 0.11 mmol). After stirring for 2 h at 80° C. under a nitrogen atmosphere, desired product could be detected by LCMS. The resulting mixture was extracted with EtOAc (3×30 mL). The combined organic layers were washed with H₂O (3×10 mL), dried over anhydrous Na₂SO₄. After filtration, the filtrate was concentrated under reduced pressure. The residue was purified by silica gel column chromatography, eluted with PE/EtOAc (1:10) to afford benzyl 4-(5-((6⁴S,3S,4S,Z)-4-((tert-butoxycarbonyl)amino)-1¹-(2-(((S)-2,2-dimethyltetrahydro-2H-pyran-4-yl)oxy)ethyl)-10,10-dimethyl-5,7-dioxo-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-1²-yl)-6-((S)-1-methoxyethyl)pyridin-3-yl) piperazine-1-carboxylate (350 mg, 24% yield) as a Brown yellow solid. LCMS (ESI): m/z [M+H] calc'd for C₆₃H₈₃N₉O₁₁ 1142.62; found 1142.5.

### Step 3

To a solution of benzyl 4-(5-((6⁴S,3S,4S,Z)-4-((tert-butoxycarbonyl)amino)-1¹-(2-(((S)-2,2-dimethyltetrahydro-2H-pyran-4-yl)oxy)ethyl)-10,10-dimethyl-5,7-dioxo-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-12-yl)-6-((S)-1-methoxyethyl)pyridin-3-yl) piperazine-1-carboxylate (330 mg, 0.29 mmol) in isopropanol (3 mL) was added Pd(OH)₂/C (162.26 mg, 1.16 mmol) under nitrogen atmosphere in a 25 mL round-bottom flask. The mixture was hydrogenated at room temperature for 5 h under hydrogen atmosphere using a hydrogen balloon, filtered through a Celite pad and concentrated under reduced pressure to afford tert-butyl ((6⁴S,3S,4S,Z)-1¹-(2-(((S)-2,2-dimethyltetrahydro-2H-pyran-4-yl)oxy)ethyl)-1²-(2-((S)-1-methoxyethyl)-5-(piperazin-1-yl) pyridin-3-yl)-10,10-dimethyl-5,7-dioxo-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-4-yl) carbamate (270 mg, crude). LCMS (ESI): m/z [M+H] calc'd for C₅₅H₇₇N₉O₉ 1008.58; found 1008.6.

### Step 4

To a stirred solution of tert-butyl ((6⁴S,3S,4S,Z)-1¹-(2-(((S)-2,2-dimethyltetrahydro-2H-pyran-4-yl)oxy)ethyl)-1²-(2-((S)-1-methoxyethyl)-5-(piperazin-1-yl) pyridin-3-yl)-10,10-dimethyl-5,7-dioxo-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-4-yl) carbamate (270 mg, 0.27 mmol) and (1-ethoxycyclopropoxy)trimethylsilane (933.58 mg, 5.36 mmol) in iPrOH (2 mL) were added AcOH (160.81 mg, 2.68 mmol) dropwise at 0° C. The resulting mixture was stirred for 15 min at 0° C. To the above mixture was added NaBH₃CN (84.14 mg, 1.34 mmol) in portions at 0° C. The resulting mixture was stirred for additional 1 h at 60° C. The reaction was quenched with saturated NaHCO₃(aq.) at 0° C. The resulting mixture was diluted with water (20 mL). The resulting mixture was extracted with EtOAc (3×30 mL). The combined organic layers were washed with H₂O (3×10 mL), dried over anhydrous Na₂SO₄. After filtration, the filtrate was concentrated under reduced pressure. The residue was purified by Prep-HPLC to afford tert-butyl ((6⁴S,3S,4S,Z)-1²-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-5,7-dioxo-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-4-yl) carbamate (130 mg, 41% yield) as a white solid. LCMS (ESI): m/z [M+H] calc'd for C₅₈H₈₁N₉O₉ 1048.62; found 1048.5.

### Step 5

To a stirred solution of tert-butyl ((6⁴S,3S,4S,Z)-1²-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-5,7-dioxo-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-4-yl) carbamate (126 mg, 0.12 mmol) in CH₂Cl₂ (5 mL) was added TFA (1 mL) dropwise at 0° C. The resulting mixture was stirred for 1 h at 25° C. The resulting mixture was concentrated under reduced pressure. This resulted in (6⁴S,3S,4S,Z)-4-amino-1²-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-5,7-dione (100 mg, crude) as a yellow solid. LCMS (ESI): m/z [M+H] calc'd for C₅₃H₇₃N₉O₇ 948.56; found 948.5.

### Step 6

To a stirred mixture of (6⁴S,3S,4S,Z)-4-amino-1²-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-5,7-dione (70 mg, 0.074 mmol) in DMF (1 mL) were added DIPEA (95.41 mg, 0.74 mmol), (1R,2S,3R)-2-methyl-3-(pyridin-2-yl) cyclopropane-1-carboxylic acid (19.62 mg, 0.11 mmol) and HATU (42.10 mg, 0.11 mmol) in portions at 0° C. The resulting mixture was stirred for 1 h at room temperature. The resulting mixture was diluted with H₂O (20 mL). The resulting mixture was extracted with EtOAc (3×20 mL), dried over anhydrous Na₂SO₄. After filtration, the filtrate was concentrated under reduced pressure. The crude product was purified by Prep-HPLC to afford (1R,2S,3R)—N-((6⁴S,3S,4S,Z)-1²-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-5,7-dioxo-3-(pyrrolidin-1-yl)-1¹H-8-oxa-6²,6³-diaza-2 (2,4)-oxazola-1(5,3)-indola-6 (2,4)-bicyclo[3.1.1]heptanacycloundecaphane-4-yl)-2-methyl-3-(pyridin-2-yl) cyclopropane-1-carboxamide (7.5 mg, 9% yield) as a white solid. LCMS (ESI): m/z [M+H] calc'd for C₆₃H₈₂N₁₀O₈ 1107.63; found 1107.6. ¹H NMR (400 MHZ, DMSO-d₆) δ 8.54-8.45 (m, 2H), 8.41 (s, 1H), 8.24 (d, J=8.4 Hz, 1H), 8.00 (s, 1H), 7.70 (q, J=9.4, 8.8 Hz, 3H), 7.45-7.35 (m, 2H), 7.26-7.17 (m, 1H), 6.37 (t, J=9.3 Hz, 1H), 5.66 (d, J=11.4 Hz, 1H), 4.57 (d, J=11.2 Hz, 1H), 4.43-4.36 (m, 1H), 4.29-4.20 (m, 1H), 4.04 (d, J=10.2 Hz, 1H), 4.00-3.90 (m, 3H), 3.78-3.72 (m, 1H), 3.66-3.56 (m, 2H), 3.55-3.48 (m, 2H), 3.45-3.37 (m, 3H) 3.28-3.15 (m, 4H), 3.00 (d, J=14.8 Hz, 1H), 2.87-2.76 (m, 3H), 2.75-2.66 (m, 4H), 2.66-2.55 (m, 4H), 2.32 (d, J=14.9 Hz, 1H), 2.24-2.02 (m, 4H), 1.75-1.62 (m, 3H), 1.60-1.53 (m, 1H), 1.52-1.38 (m, 4H), 1.34 (d, J=6.3 Hz, 3H), 1.06 (s, 3H), 1.04-0.88 (m, 8H), 0.88-0.80 (m, 1H), 0.56 (d, J=12.6 Hz, 5H), 0.48-0.40 (m, 2H), 0.37-0.28 (m, 2H).

### Intermediate 6. Synthesis of (1S,2R,3S)-2-methyl-3-(pyrimidin-4-yl) cyclopropane-1-carboxylic acid

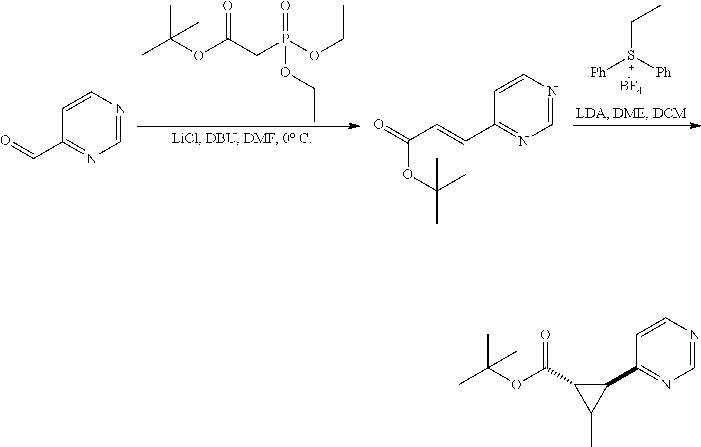

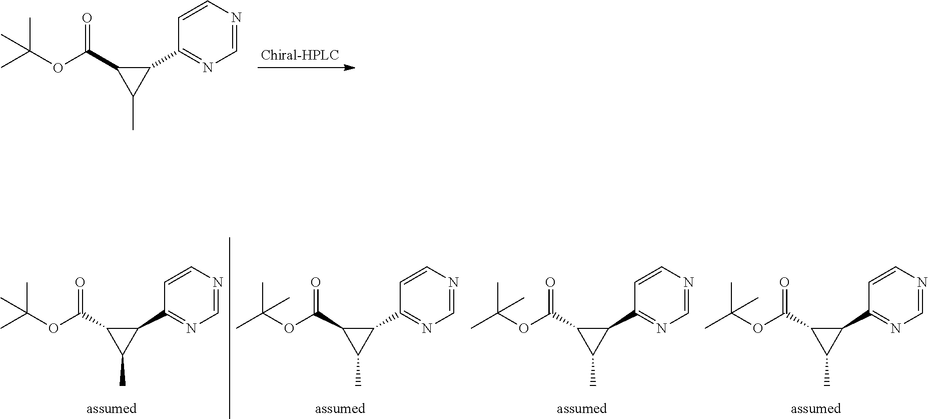

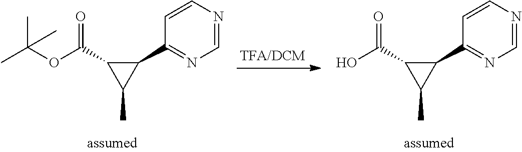 [SMILES: C[C@H]1[C@H](C(=O)OC(C)(C)C)[C@H]1c1ccncn1 ; C[C@H]1[C@H](C(=O)O)[C@H]1c1ccncn1]

### Step 1

To a stirred solution of LiCl (0.31 g, 7.401 mmol) and pyrimidine-4-carbaldehyde (1 g, 9.251 mmol) in DMF were added DBU (1.69 g, 11.101 mmol) and tert-butyl 2-(diethoxyphosphoryl)acetate (2.80 g, 11.101 mmol) dropwise at 0° C. under an air atmosphere. The resulting mixture was stirred for an additional 1 h at room temperature. The reaction was monitored by LCMS. The reaction was quenched with sat. aq. NH₄Cl at 0° C. The resulting mixture was concentrated under reduced pressure. The resulting mixture was diluted with EtOAc (200 mL). The resulting mixture was washed with brine (3×200 mL). The resulting mixture was concentrated under reduced pressure. The residue was purified by silica gel column chromatography, eluted with PE/EtOAc (10:1) to afford tert-butyl (E)-3-(pyrimidin-4-yl) acrylate (1 g, 52.41%) as a yellow solid. LCMS (ESI): m/z [M+H] calc'd for C₁₁H₁₄N₂O₂ 206.11; found 206.11.

### Step 2

A solution of ethyldiphenylsulfanium (2.192 g, 10.182 mmol) in DME: CH₂Cl₂=10:1 was treated with LDA (2M in THF) (6 mL, 11.879 mmol) for 0.5 h at −78° C. under a nitrogen atmosphere followed by the addition of tert-butyl (E)-3-(pyrimidin-4-yl) acrylate (700 mg, 3.394 mmol) dropwise at room temperature. The resulting mixture was stirred for an additional 1 h at room temperature. The reaction was monitored by LCMS. The reaction was quenched with sat. aq. NH₄Cl at room temperature. The aqueous layer was extracted with EtOAc (3×100 mL). The resulting mixture was concentrated under reduced pressure to afford tert-butyl (1 S,2R,3S)-2-methyl-3-(pyrimidin-4-yl) cyclopropane-1-carboxylate (400 mg, 53.7%) as a yellow solid. LCMS (ESI): m/z [M+H] calc'd for C₇H₁₀O₂ 126.1; found 126.1. The crude product (400 mg) was purified by Prep-HPLC to afford tert-butyl (1 S,2R,3S)-2-methyl-3-(pyrimidin-4-yl) cyclopropane-1-carboxylate (55 mg, 13.75%) as a white solid. LCMS (ESI): m/z [M+H] calc'd for C₁₃H₁₈N₂O₂ 234.14; found 234.14.

### Step 3

A solution/mixture of tert-butyl (1S,2R,3S)-2-methyl-3-(pyrimidin-4-yl) cyclopropane-1-carboxylate, (50 mg, 0.213 mmol) and TFA (5 mL, 67.315 mmol) in CH₂Cl₂ was stirred for 1 h at room temperature under air atmosphere. The reaction was monitored by LCMS. The resulting mixture was concentrated under reduced pressure. The crude product V-1380-3Q was used in the next step directly without further purification (single diastereomer of unknown absolute configuration Isomer). LCMS (ESI): m/z [M+H] calc'd for C₉H₁₀N₂O₂ 178.07; found 178.07.

### Example 5: Synthesis of (1R,2S,3R)—N-(6³S,3R,4S)-1²-(5-(4-cyclopropylpiperazin-1-yl)-2-((S)-1-methoxyethyl)pyridin-3-yl)-3-ethoxy-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2 (5,2)-oxazola-1(5,3)-indola-6(1,3)-pyridazinacycloundecaphane-4-yl)-2-methyl-3-(pyrimidin-4-yl) cyclopropane-1-carboxamide

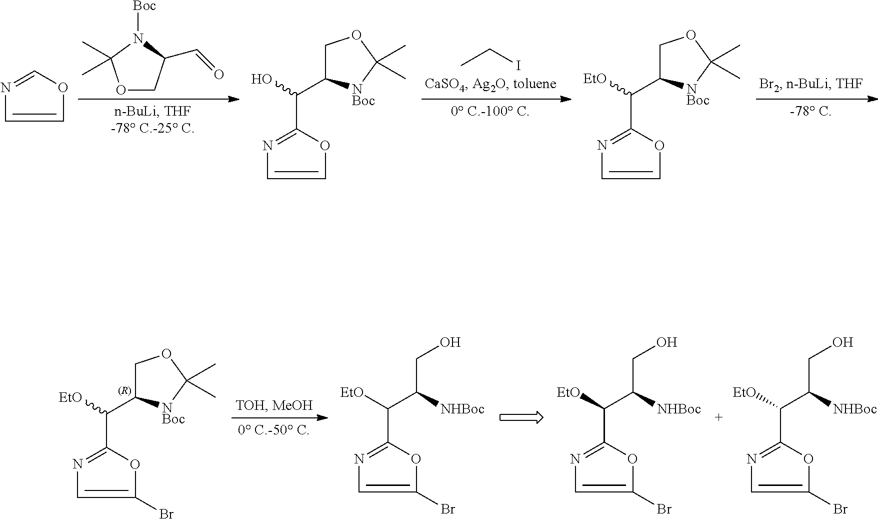

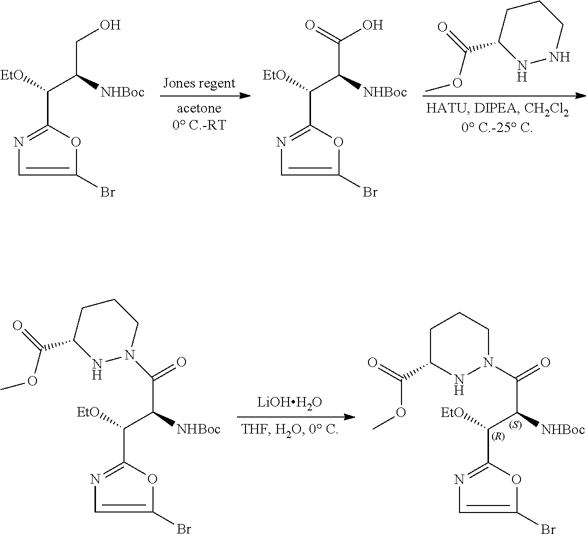

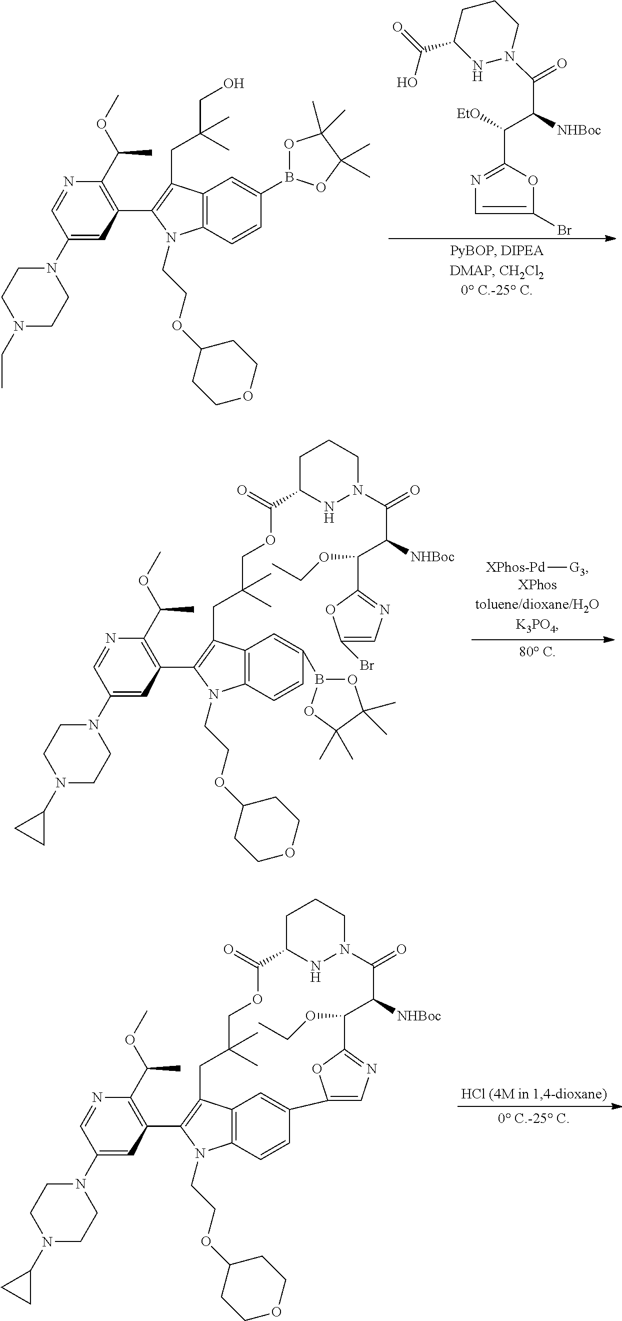

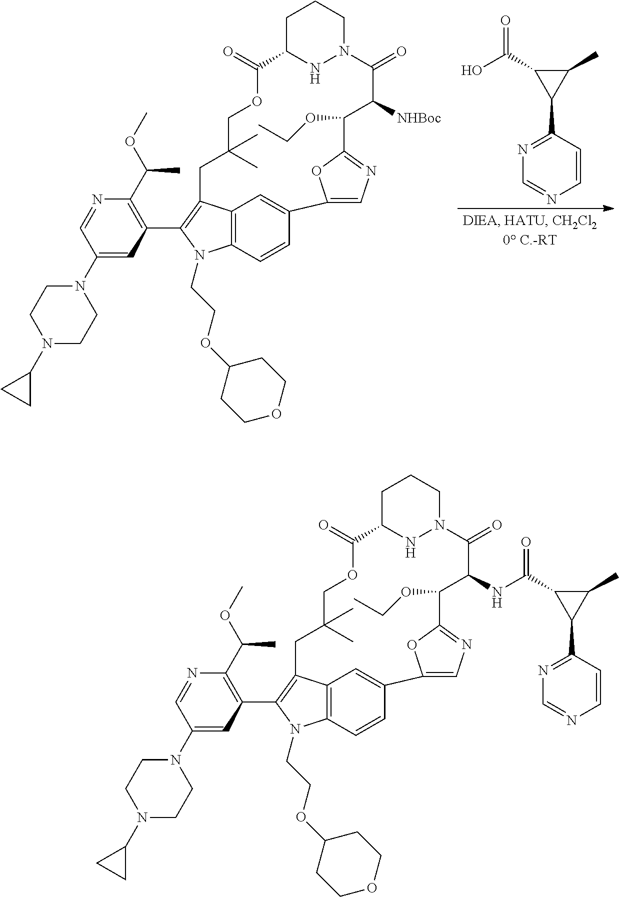

### Step 1

To a stirred solution of oxazole (5 g, 72.40 mmol,) in THF (70 mL) were added n-butyl lithium (34.75 mL, 86.88 mmol) dropwise at −78° C. under a nitrogen atmosphere. The resulting mixture was stirred for additional 30 min at −78° C. To the above mixture was added tert-butyl (R)-4-formyl-2,2-dimethyloxazolidine-3-carboxylate (19.92 g, 86.88 mmol) dropwise at −78° C. The resulting mixture was stirred for additional 16 h at 25° C. The reaction was quenched with sat. NH₄Cl (aq.) at 0° C. The resulting mixture was extracted with CH₂Cl₂ (3×100 mL), dried over anhydrous Na₂SO₄. After filtration, the filtrate was concentrated under reduced pressure. The residue was purified by Prep-HPLC this resulted in tert-butyl (4R)-4-(hydroxy (oxazol-2-yl)methyl)-2,2-dimethyloxazolidine-3-carboxylate (8.07 g, 37.3%) as a yellow oil. LCMS (ESI): m/z [M+H] calc'd for C₁₄H₂₂N₂O₅ 299.15; found 299.20.

### Step 2

To a stirred mixture of tert-butyl (4R)-4-(hydroxy (oxazol-2-yl)methyl)-2,2-dimethyloxazolidine-3-carboxylate (8.01 g, 26.85 mmol) and iodoethane (41.87 g, 268.49 mmol) in toluene (80 mL) were added calcium sulfate (10.96 g, 80.55 mmol) and Ag₂O (62.22 g, 268.49 mmol) in portions at 0° C. under a nitrogen atmosphere. The resulting mixture was stirred for additional 3 h at 100° C. The resulting mixture was filtered, the filter cake was washed with CH₂Cl₂ (3×30 mL). The filtrate was concentrated under reduced pressure. The residue was purified by Prep-HPLC to afford tert-butyl (4R)-4-(ethoxy (oxazol-2-yl)methyl)-2,2-dimethyloxazolidine-3-carboxylate (6.74 g, 76.9%) as a yellow liquid. LCMS (ESI): m/z [M+H] calc'd for C₁₆H₂₆N₂O₅ 327.18; found 327.10.

### Step 3

In a 250-mL round bottom flask, to a solution of tert-butyl (4R)-4-(ethoxy (oxazol-2-yl)methyl)-2,2-dimethyloxazolidine-3-carboxylate (4.1 g, 12.56 mmol) in THF (40 mL) was added dropwise n-BuLi (0.97 g, 15.07 mmol) at −78° C. under a nitrogen atmosphere. The reaction mixture was stirred at −78° C. for 40 min. Then a solution of Br₂ (2.41 g, 15.07 mmol) in 10 mL THF was added dropwise and the mixture was stirred for another 2 h. The reaction was quenched with sat. NH₄Cl (aq.) at 0° C. The resulting mixture was extracted with CH₂Cl₂ (3×100 mL), dried over anhydrous Na₂SO₄. After filtration, the filtrate was concentrated under reduced pressure. The residue was purified by Prep-HPLC to afford tert-butyl (4R)-4-((5-bromooxazol-2-yl)(ethoxy)methyl)-2,2-dimethyloxazolidine-3-carboxylate (3.59 g, 70.5%) as a yellow oil. LCMS (ESI): m/z [M+H] calc'd for C₁₆H₂₅BrN₂O₅ 405.09; found 304.95.

### Step 4

To a stirred solution of tert-butyl (4R)-4-((5-bromooxazol-2-yl) (ethoxy)methyl)-2,2-dimethyloxazolidine-3-carboxylate (3.575 g, 8.82 mmol) in MeOH (40 mL) were added TsOH (0.15 g, 0.88 mmol) in portions at 0° C. The resulting mixture was stirred overnight at 50° C. The resulting mixture was concentrated under reduced pressure. The resulting mixture was extracted with CH₂Cl₂ (3×100 mL), dried over anhydrous Na₂SO₄. After filtration, the filtrate was concentrated under reduced pressure. The residue was purified by Prep-HPLC followed by chiral HPLC to afford tert-butyl ((2R)-1-(5-bromooxazol-2-yl)-1-ethoxy-3-hydroxypropan-2-yl) carbamate A (530 mg, 30.6%) and tert-butyl ((2R)-1-(5-bromooxazol-2-yl)-1-ethoxy-3-hydroxypropan-2-yl) carbamate B (1.02 g, 58.9%) as yellow oils. LCMS (ESI): m/z [M+Na] calc'd for C₁₃H₂₁BrN₂O₅ 386.05; found 387.10.

### Step 5

To a stirred solution of tert-butyl ((1R,2R)-1-(5-bromooxazol-2-yl)-1-ethoxy-3-hydroxypropan-2-yl) carbamate (1 g, 2.74 mmol) in acetone (10 mL) were added Jones reagent (2.74 mL, 5.48 mmol) dropwise at 0° C. The resulting mixture was stirred for additional 1 h at room temperature. The reaction was quenched with iPrOH at 0° C. The mixture was basified to pH 8 with saturated NaHCO₃(aq.). The resulting mixture was filtered, the filter cake was washed with MeOH (3×10 mL). The filtrate was concentrated under reduced pressure. The mixture was acidified to pH 6 with citric acid. The resulting mixture was extracted with CH₂Cl₂ (3×100 mL), dried over anhydrous Na₂SO₄. After filtration, the filtrate was concentrated under reduced pressure. The residue was purified by Prep-HPLC to afford (2S,3R)-3-(5-bromooxazol-2-yl)-2-((tert-butoxycarbonyl)amino)-3-ethoxypropanoic acid (510 mg, 49.1%) as a light yellow solid. LCMS (ESI): m/z [M+Na] calc'd for C₁₃H₁₉BrN₂O₆ 399.04; found 400.95.

### Step 6

To a stirred solution of (2S,3R)-3-(5-bromooxazol-2-yl)-2-((tert-butoxycarbonyl)amino)-3-ethoxypropanoic acid (211 mg, 0.56 mmol) and methyl(S)-hexahydropyridazine-3-carboxylate (210.98 mg, 1.46 mmol) in CH₂Cl₂ (5 mL) were added DIPEA (719.16 mg, 5.56 mmol) and HATU (211.57 mg, 0.56 mmol) in portions at 0° C. The resulting mixture was stirred for additional 1 h at 25° C. The resulting mixture was extracted with CH₂Cl₂ (3×50 mL), dried over anhydrous Na₂SO₄. After filtration, the filtrate was concentrated under reduced pressure. The residue was purified by Prep-HPLC to afford methyl(S)-1-((2S,3R)-3-(5-bromooxazol-2-yl)-2-((tert-butoxycarbonyl)amino)-3-ethoxypropanoyl) hexahydropyridazine-3-carboxylate (195 mg, 69.3%) as a light yellow solid. LCMS (ESI): m/z [M+H] calc'd for C₁₉H₂₉BrN₄O₇ 505.12; found 505.1.

### Step 7

To a stirred solution of methyl(S)-1-((2S,3R)-3-(5-bromooxazol-2-yl)-2-((tert-butoxycarbonyl)amino)-3-ethoxypropanoyl) hexahydropyridazine-3-carboxylate (191 mg, 0.38 mmol) in THF (3 mL) and H₂O (3 mL) were added LiOH·H₂O (79.29 mg, 1.89 mmol) in portions at 0° C. The resulting mixture was stirred for additional 1 h at 0° C. The mixture was acidified to pH 6 with citric acid. The resulting mixture was extracted with CH₂Cl₂ (3×50 mL), dried over anhydrous Na₂SO₄. After filtration, the filtrate was concentrated under reduced pressure. The crude product was used in the next step directly without further purification. This resulted in(S)-1-((2S,3R)-3-(5-bromooxazol-2-yl)-2-((tert-butoxycarbonyl)amino)-3-ethoxypropanoyl) hexahydropyridazine-3-carboxylic acid (196 mg, crude) as a yellow solid. LCMS (ESI): m/z [M+H] calc'd for C₁₈H₂₇BrN₄O₇ 490.11; found 491.10.

### Step 8

To a stirred solution of(S)-3-(2-(5-(4-cyclopropylpiperazin-1-yl)-2-(1-methoxyethyl)pyridin-3-yl)-1-(2-((tetrahydro-2H-pyran-4-yl)oxy)ethyl)-5-(4,4,5,5-tetramethyl-1,3,2-dioxaborolan-2-yl)-1H-indol-3-yl)-2,2-dimethylpropan-1-ol (247 mg, 0.35 mmol) and (S)-1-((2S,3R)-3-(5-bromooxazol-2-yl)-2-((tert-butoxycarbonyl)amino)-3-ethoxypropanoyl) hexahydropyridazine-3-carboxylic acid (186.25 mg, 0.38 mmol) in CH₂Cl₂ (3 mL) were added DIPEA (445.39 mg, 3.45 mmol), DMAP (63.15 mg, 0.52 mmol) and PyBOP (717.32 mg, 1.38 mmol) in portions at 0° C. The resulting mixture was stirred for additional 2 h at 25° C. The resulting mixture was extracted with EtOAc (3×100 mL), dried over anhydrous Na₂SO₄. After filtration, the filtrate was concentrated under reduced pressure. The residue was purified by silica gel column chromatography, eluted with PE/EtOAc (1:1) to afford 3-(2-(5-(4-cyclopropylpiperazin-1-yl)-2-((S)-1-methoxyethyl)pyridin-3-yl)-1-(2-((tetrahydro-2H-pyran-4-yl)oxy)ethyl)-5-(4,4,5,5-tetramethyl-1,3,2-dioxaborolan-2-yl)-1H-indol-3-yl)-2,2-dimethylpropyl(S)-1-((2S,3R)-3-(5-bromooxazol-2-yl)-2-((tert-butoxycarbonyl)amino)-3-ethoxypropanoyl) hexahydropyridazine-3-carboxylate (663 mg, crude) as a brown solid. LCMS (ESI): m/z [M+H] calc'd for C₅₉H₈₆BBrN₈O₁₂ 1189.56; found 1189.6.

### Step 9

To a stirred solution of 3-(2-(5-(4-cyclopropylpiperazin-1-yl)-2-((S)-1-methoxyethyl)pyridin-3-yl)-1-(2-((tetrahydro-2H-pyran-4-yl)oxy)ethyl)-5-(4,4,5,5-tetramethyl-1,3,2-dioxaborolan-2-yl)-1H-indol-3-yl)-2,2-dimethylpropyl(S)-1-((2S,3R)-3-(5-bromooxazol-2-yl)-2-((tert-butoxycarbonyl)amino)-3-ethoxypropanoyl) hexahydropyridazine-3-carboxylate (635 mg, 0.53 mmol) in toluene (6 mL), 1,4-dioxane (2 mL) and H₂O (2 mL) were added K₃PO₄ (339.77 mg, 1.60 mmol), XPhos (25.44 mg, 0.05 mmol) and XPhos Pd G3 (45.16 mg, 0.05 mmol) in portions at 25° C. under a nitrogen atmosphere. The resulting mixture was stirred for additional 2 h at 80° C. The resulting mixture was extracted with EtOAc (3×50 mL), dried over anhydrous Na₂SO₄. After filtration, the filtrate was concentrated under reduced pressure. The residue was purified by silica gel column chromatography, eluted with PE/EtOAc (1:1) to afford tert-butyl ((6³S,3R,4S)-1²-(5-(4-cyclopropylpiperazin-1-yl)-2-((S)-1-methoxyethyl)pyridin-3-yl)-3-ethoxy-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2 (5,2)-oxazola-1(5,3)-indola-6(1,3)-pyridazinacycloundecaphane-4-yl) carbamate (86 mg, 16.3%) as a light yellow solid. LCMS (ESI): m/z [M+H] calc'd for C₅₃H₇₄N₈O₁₀ 983.55; found 983.4.

### Step 10

Into a 8 mL vial were added tert-butyl ((6³S,3R,4S)-1²-(5-(4-cyclopropylpiperazin-1-yl)-2-((S)-1-methoxyethyl)pyridin-3-yl)-3-ethoxy-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2 (5,2)-oxazola-1(5,3)-indola-6(1,3)-pyridazinacycloundecaphane-4-yl) carbamate (82 mg, 0.08 mmol) and HCl(gas) in 1,4-dioxane (2 mL) at 0° C. The resulting mixture was stirred for additional 30 min at 25° C. The resulting mixture was concentrated under reduced pressure. The crude product was used in the next step directly without further purification. This resulted in (6³S,3R,4S)-4-amino-1²-(5-(4-cyclopropylpiperazin-1-yl)-2-((S)-1-methoxyethyl)pyridin-3-yl)-3-ethoxy-10,10-dimethyl-1¹-(2-((tetrahydro-2H-pyran-4-yl)oxy)ethyl)-6¹,6²,6³,6⁴,6⁵,6⁶-hexahydro-1¹H-8-oxa-2 (5,2)-oxazola-1(5,3)-indola-6(1,3)-pyridazinacycloundecaphane-5,7-dione (118 mg, crude) as a yellow green solid. LCMS (ESI): m/z [M+H] calc'd for C₄₈H₆₆N₈O₈ 883.50; found 883.55.

### Step 11

To a stirred solution of (6³S,3R,4S)-4-amino-1²-(5-(4-cyclopropylpiperazin-1-yl)-2-((S)-1-methoxyethyl)pyridin-3-yl)-3-ethoxy-10,10-dimethyl-1¹-(2-((tetrahydro-2H-pyran-4-yl)oxy)ethyl)-6¹,6²,6³,6⁴,6⁵,6⁶-hexahydro-1¹H-8-oxa-2 (5,2)-oxazola-1(5,3)-indola-6(1,3)-pyridazinacycloundecaphane-5,7-dione (118 mg, 0.13 mmol) and (1R,2S,3R)-2-methyl-3-(pyrimidin-4-yl) cyclopropane-1-carboxylic acid (73.70 mg, 0.17 mmol) in CH₂Cl₂ (5 mL) were added DIPEA (172.70 mg, 1.34 mmol) and HATU (50.81 mg, 0.13 mmol) in portions at 0° C. The resulting mixture was stirred for additional 1 h at room temperature. The resulting mixture was concentrated under reduced pressure. The crude product was purified by Prep-HPLC to afford (1R,2S,3R)—N-((6³S,3R,4S)-1²-(5-(4-cyclopropylpiperazin-1-yl)-2-((S)-1-methoxyethyl)pyridin-3-yl)-3-ethoxy-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2 (5,2)-oxazola-1(5,3)-indola-6(1,3)-pyridazinacycloundecaphane-4-yl)-2-methyl-3-(pyrimidin-4-yl) cyclopropane-1-carboxamide (16.8 mg, 12.0%) as a white solid. LCMS (ESI): m/z [M+H] calc'd for C₅₇H₇₄N₁₀O₉ 1043.56; found 1043.65. ¹H NMR (400 MHZ, DMSO-d6) δ 9.06 (s, 1H), 8.64 (t, J=7.8 Hz, 2H), 8.53 (s, 1H), 8.45 (d, J=2.8 Hz, 1H), 7.95 (s, 1H), 7.65 (q, J=8.6 Hz, 2H), 7.56 (d, J=5.3 Hz, 1H), 7.17 (d, J=2.9 Hz, 1H), 6.17 (t, J=7.7 Hz, 1H), 4.83 (d, J=11.6 Hz, 1H), 4.60 (t, J=11.9 Hz, 1H), 4.51-4.22 (m, 4H), 4.09 (q, J=6.1 Hz, 1H), 3.67 (d, J=10.9 Hz, 1H), 3.62-3.47 (m, 3H), 3.33-3.07 (m, 11H), 3.02 (td, J=6.6, 3.6 Hz, 2H), 2.88 (d, J=14.4 Hz, 2H), 2.82-2.63 (m, 4H), 2.59 (t, J=5.1 Hz, 1H), 2.18-2.02 (m, 1H), 2.00-1.57 (m, 6H), 1.50 (d, J=14.9 Hz, 3H), 1.33 (d, J=6.1 Hz, 3H), 1.23 (d, J=5.6 Hz, 1H), 1.25-1.01 (m, 9H), 0.90 (s, 3H). 0.90 (s, 2H). 0.54-0.03 (m, 7H).

### Example 6: Synthesis of (1R,2S,3R)—N-((6³S,3S,4S,Z)-3-((S)-3-cyanomorpholino)-1²-(2-((S)-1-methoxyethyl)pyridin-3-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methyl-3-(pyrimidin-4-yl) cyclopropane-1-carboxamide

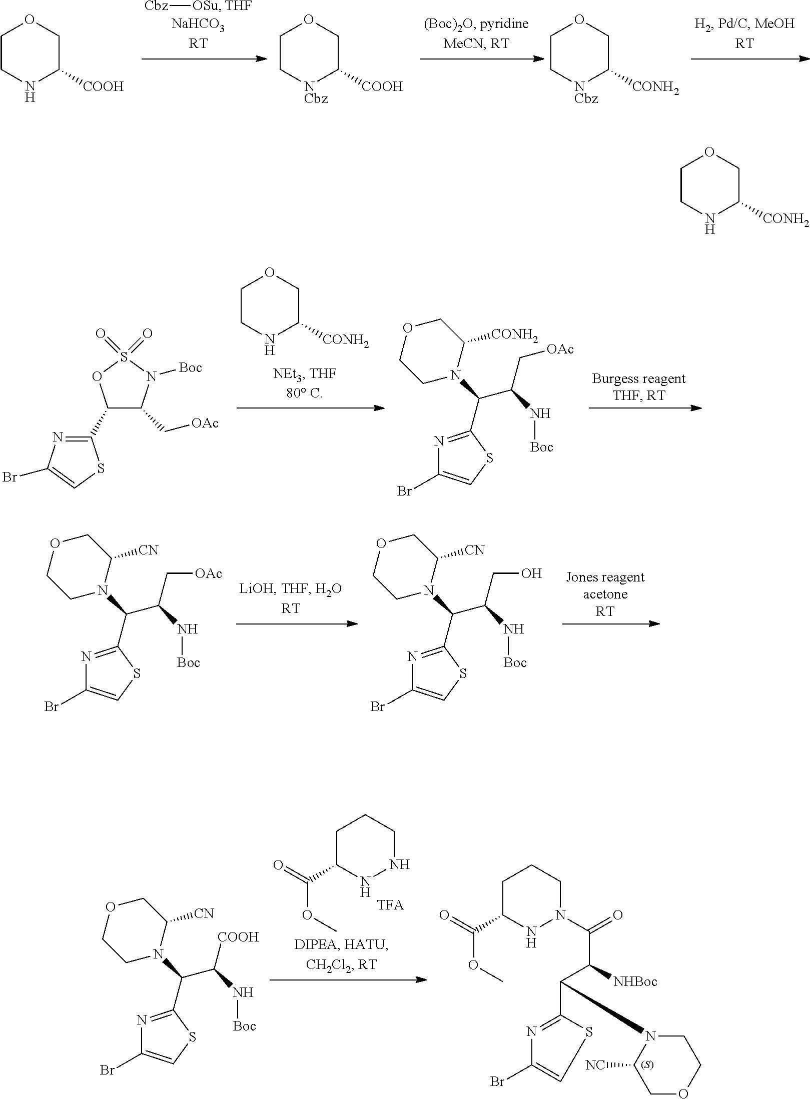

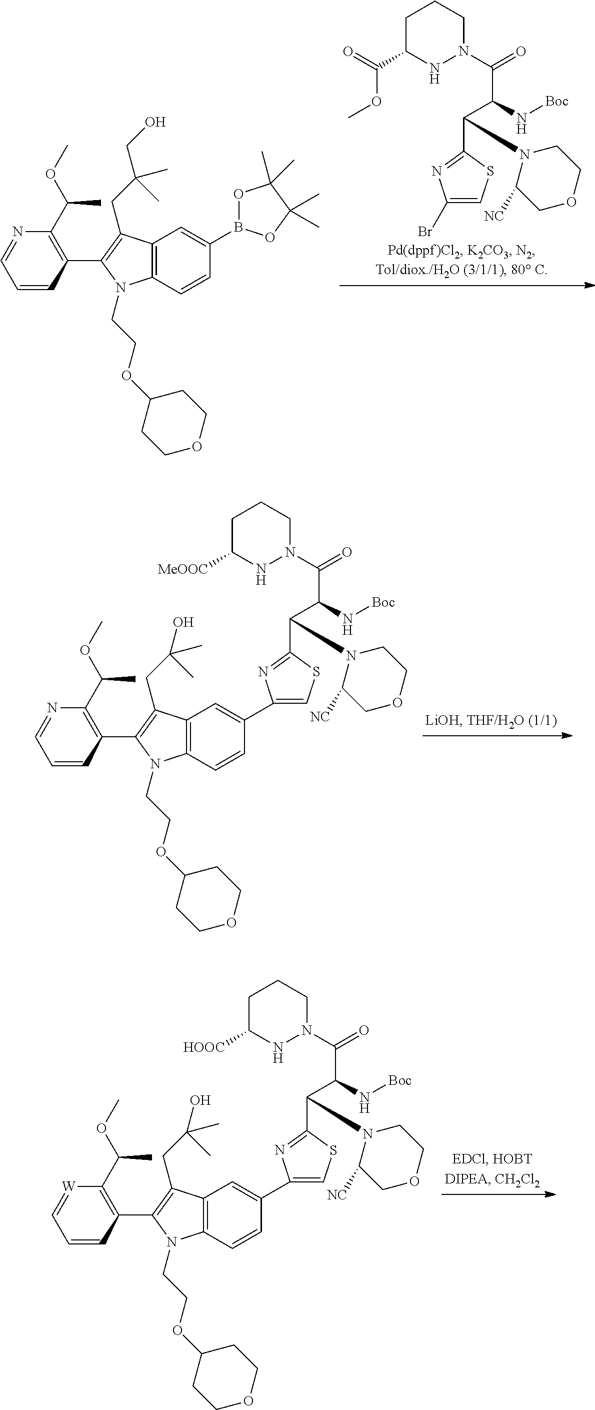

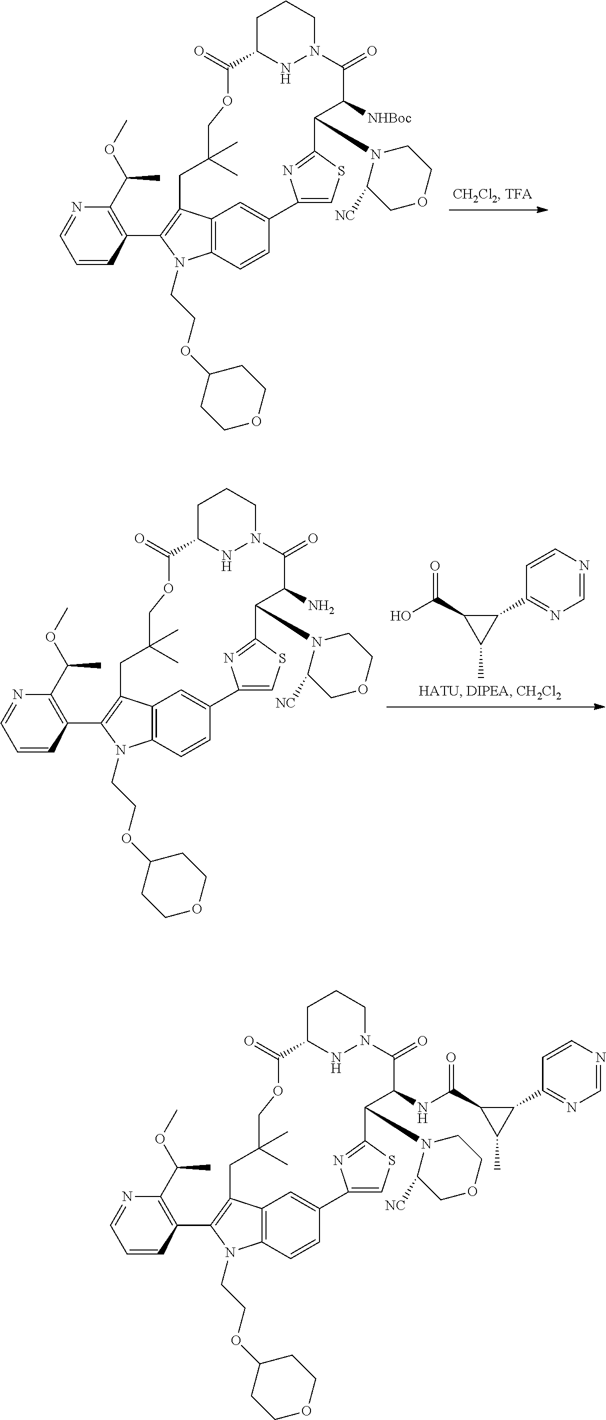

### Step 1

To a stirred mixture of (3R)-morpholine-3-carboxylic acid (6.5 g, 49.57 mmol) in THF (35 mL) and saturated NaHCO₃(aq.) (35 mL) was added 4-N-Cbz-cyclohexanone (15.94 g, 64.44 mmol) in portions at 0° C. The resulting mixture was stirred for 1 h at room temperature. The resulting mixture was diluted with H₂O (40 mL). The aqueous layer was extracted with CH₂Cl₂ (3×10 mL). The resulting mixture was concentrated under reduced pressure. The residue product was purified by Prep-HPLC to afford (3R)-4-[(benzyloxy) carbonyl]morpholine-3-carboxylic acid (8 g, 55.9%) as a colorless oil. LCMS (ESI): m/z [M+Na] calc'd for C₁₃H₁₅NO₅ 288.10; found 288.0.

### Step 2

To a stirred mixture of (3R)-4-[(benzyloxy) carbonyl]morpholine-3-carboxylic acid (8 g, 30.16 mmol) and Boc₂O (8.39 mL, 39.21 mmol) in MeCN (80 mL) were added NH₄HCO₃ (2.62 g, 33.18 mmol) and pyridine (1.6 mL, 20.23 mmol) in portions at 0° C. The resulting mixture was stirred for 3 h at room temperature. The resulting mixture was diluted with H₂O (30 mL). The aqueous layer was extracted with CH₂Cl₂ (3×15 mL). The resulting mixture was concentrated under reduced pressure. The crude product was purified by Prep-HPLC to afford benzyl (3R)-3-carbamoylmorpholine-4-carboxylate (6.92 g, 86.8%) as a yellow solid. LCMS (ESI): m/z [M+H] calc'd for C₁₃H₁₆N₂O₄ 265.11; found 265.0.

### Step 3

To a stirred mixture of benzyl (R)-3-carbamoylmorpholine-4-carboxylate (6.92 g, 26.18 mmol) in MeOH (70 mL) was added Pd/C (3.5 g, 32.89 mmol) at room temperature. The resulting mixture was stirred for 1 h at room temperature under a hydrogen atmosphere. The resulting mixture was filtered, the filter cake was washed with MeOH (2×20 mL). The filtrate was concentrated under reduced pressure. This resulted in (R)-morpholine-3-carboxamide (2.9 g, 65.6%) as a white solid. LCMS (ESI): m/z [M+H] calc'd for C₅H₁₀N₂O₂ 131.07, found 131.0.

### Step 4

To a stirred mixture of tert-butyl (4R,5R)-4-(acetoxymethyl)-5-(4-bromothiazol-2-yl)-1,2,3-oxathiazolidine-3-carboxylate 2,2-dioxide (5 g, 10.93 mmol) in THF (50 mL) were added (R)-morpholine-3-carboxamide (2.8 g, 21.51 mmol) and NEt₃ (7.60 mL, 54.67 mmol) in portions at 0° C. The resulting mixture was stirred for 24 h at 80° C. The resulting mixture was concentrated under reduced pressure. The residue was purified by silica gel column chromatography, eluted with EtOAc, to afford (2S,3S)-3-(4-bromothiazol-2-yl)-2-((tert-butoxycarbonyl)amino)-3-((R)-3-carbamoylmorpholino) propyl acetate (3.15 g, 54.5%) as a yellow solid. ESI-MS m/z=507.0 [M+H]⁺; Calculated MW: 506.1. LCMS (ESI): m/z [M+H] calc'd for C₁₈H₂₇BrN₄O₆S 507.08, found 507.0.

### Step 5

To a stirred solution of (2S,3S)-3-(4-bromothiazol-2-yl)-2-((tert-butoxycarbonyl)amino)-3-((R)-3-carbamoylmorpholino) propyl acetate (3 g, 5.91 mmol) in THF (30 mL) was added Burgess reagent (2113.42 mg, 8.87 mmol) in portions at 0° C. The resulting mixture was stirred for 2 h at room temperature. The resulting mixture was diluted with H₂O (15 mL). The aqueous layer was extracted with CH₂Cl₂ (3×10 mL). The resulting mixture was concentrated under reduced pressure. This resulted in (2S,3S)-3-(4-bromothiazol-2-yl)-2-((tert-butoxycarbonyl)amino)-3-((S)-3-cyanomorpholino) propyl acetate (3.4 g, crude) as a yellow solid. LCMS (ESI): m/z [M+Na] calc'd for C₁₈H₂₅BrN₄O₅S 511.07, found 511.0.

### Step 6

To a stirred mixture of (2S,3S)-3-(4-bromothiazol-2-yl)-2-((tert-butoxycarbonyl)amino)-3-((S)-3-cyanomorpholino) propyl acetate (3.4 g, 6.95 mmol) in THF (20 mL) and H₂O (20 mL) was added LiOH·H₂O (1.46 g, 34.74 mmol) in portions at 0° C. The resulting mixture was stirred for 1 h at room temperature. The resulting mixture was diluted with CH2Cl2 (15 mL). The aqueous layer was extracted with CH₂Cl₂ (3×10 mL). The resulting mixture was concentrated under reduced pressure. This resulted in tert-butyl ((1 S,2S)-1-(4-bromothiazol-2-yl)-1-((S)-3-cyanomorpholino)-3-hydroxypropan-2-yl) carbamate (2.2 g, 70.7%) as a yellow oil. LCMS (ESI): m/z [M+H] calc'd for C₁₆H₂₃BrN₄O₄S 447.06, found 448.9.

### Step 7

To a stirred mixture of tert-butyl ((1S,2S)-1-(4-bromothiazol-2-yl)-1-((S)-3-cyanomorpholino)-3-hydroxypropan-2-yl) carbamate (2.2 g, 4.92 mmol) in acetone (22 mL) was added Jones reagent (1.95 g, 9.85 mmol) dropwise at 0° C. The resulting mixture was stirred for 1 h at room temperature. The reaction was quenched with iPrOH at 0° C. The reaction was quenched by the addition of NaHCO₃ at 0° C. The residue was basified to pH 6 with citric acid. The aqueous layer was extracted with CH₂Cl₂ (3×10 mL). The resulting mixture was concentrated under reduced pressure. The residue product was purified by Prep-HPLC to afford (2S,3S)-3-(4-bromothiazol-2-yl)-2-((tert-butoxycarbonyl)amino)-3-((S)-3-cyanomorpholino) propanoic acid (680 mg, 25.3%) as a white solid. LCMS (ESI): m/z [M+H] calc'd for C₁₆H₂₁BrN₄O₅S 461.04, found 461.0.

### Step 8

To a stirred solution of (2S,3S)-3-(4-bromothiazol-2-yl)-2-((tert-butoxycarbonyl)amino)-3-((S)-3-cyanomorpholino) propanoic acid (680 mg, 1.47 mmol) and methyl(S)-hexahydropyridazine-3-carboxylate TFA salt (287.75 mg, 2.21 mmol) in CH₂Cl₂ (7 mL) were added DIPEA (1.910 g, 14.74 mmol) and HATU (840.70 mg, 2.21 mmol) in portions at 0° C. The resulting mixture was stirred for 1 h at room temperature. The resulting mixture was diluted with H₂O (10 mL) and extracted with CH₂Cl₂ (3×10 mL). The resulting mixture was concentrated under reduced pressure. The residue was purified by silica gel column chromatography, eluted with PE/EtOAc (6:1) to afford methyl(S)-1-((2S,3S)-3-(4-bromothiazol-2-yl)-2-((tert-butoxycarbonyl)amino)-3-((S)-3-cyanomorpholino) propanoyl) hexahydropyridazine-3-carboxylate (570 mg, 55.9%) as a yellow solid. LCMS (ESI): m/z [M+H] calc'd for C₂H₃₁BrN₆O₆S 587.12, found 587.0.

### Step 9

To a 100 mL flask equipped with a stir bar was charged with(S)-3-(2-(2-(1-methoxyethyl)pyridin-3-yl)-1-(2-((tetrahydro-2H-pyran-4-yl)oxy)ethyl)-5-(4,4,5,5-tetramethyl-1,3,2-dioxaborolan-2-yl)-1H-indol-3-yl)-2,2-dimethylpropan-1-ol (300 mg, 0.51 mmol), methyl(S)-1-((2S,3S)-3-(4-bromothiazol-2-yl)-2-((tert-butoxycarbonyl)amino)-3-((S)-3-cyanomorpholino) propanoyl) hexahydropyridazine-3-carboxylate (297.42 mg, 0.51 mmol), Pd(dppf)Cl₂ (74.09 mg, 0.10 mmol) and K₃PO₄ (268.65 mg, 1.27 mmol) at room temperature. The vial was sealed with a rubber stopper, then vacuumed and refilled with nitrogen for three cycles. Toluene (4.5 mL), 1,4-dioxane (1.5 mL), H₂O (1.5 mL) were then added. The resulting mixture was stirred at 80° C. for 2 hours. The aqueous layer was extracted with EtOAc (3×100 mL). Solvent was removed under reduced pressure to give crude product, which was purified on silica gel column chromatography using PE/EtOAc (3:1) to afford methyl(S)-1-((2S,3S)-2-((tert-butoxycarbonyl)amino)-3-((S)-3-cyanomorpholino)-3-(4-(3-(2-hydroxy-2-methylpropyl)-2-(2-((S)-1-methoxyethyl)pyridin-3-yl)-1-(2-((tetrahydro-2H-pyran-4-yl)oxy)ethyl)-1H-indol-5-yl) thiazol-2-yl) propanoyl) hexahydropyridazine-3-carboxylate (157 mg, 32.0%) as a yellow solid. LCMS (ESI): m/z [M+H] calc'd for C₄₉H₆₆N₈O₁₀S 959.46, found 959.3.

### Step 10

Into a 40 mL vial were added methyl(S)-1-((2S,3S)-2-((tert-butoxycarbonyl)amino)-3-((S)-3-cyanomorpholino)-3-(4-(3-(2-hydroxy-2-methylpropyl)-2-(2-((S)-1-methoxyethyl)pyridin-3-yl)-1-(2-((tetrahydro-2H-pyran-4-yl)oxy)ethyl)-1H-indol-5-yl) thiazol-2-yl) propanoyl) hexahydropyridazine-3-carboxylate (147 mg, 0.15 mmol), LiOH (11.01 mg, 0.46 mmol) and H₂O (2 mL)/THF (2 mL) at room temperature. The resulting mixture was stirred for 30 min at 0° C. under an argon atmosphere. The reaction was quenched with H₂O at 0° C., acidified to pH 6 then extracted with EtOAc (3×20 mL). The combined organic layers were dried over anhydrous Na₂SO₄. After filtration, the filtrate was concentrated under reduced pressure. This resulted in(S)-1-((2S,3S)-2-((tert-butoxycarbonyl)amino)-3-((S)-3-cyanomorpholino)-3-(4-(3-(2-hydroxy-2-methylpropyl)-2-(2-((S)-1-methoxyethyl)pyridin-3-yl)-1-(2-((tetrahydro-2H-pyran-4-yl)oxy)ethyl)-1H-indol-5-yl) thiazol-2-yl) propanoyl) hexahydropyridazine-3-carboxylic acid (135 mg, 93.0% yield) as a yellow solid. LCMS (ESI): m/z [M+H] calc'd for C₄₈H₆₄N₈O₁₀S 945.45, found 945.3.

### Step 11

Into a 100 mL round-bottom flask were added(S)-1-((2S,3S)-2-((tert-butoxycarbonyl)amino)-3-((S)-3-cyanomorpholino)-3-(4-(3-(2-hydroxy-2-methylpropyl)-2-(2-((S)-1-methoxyethyl)pyridin-3-yl)-1-(2-((tetrahydro-2H-pyran-4-yl)oxy)ethyl)-1H-indol-5-yl) thiazol-2-yl) propanoyl) hexahydropyridazine-3-carboxylic acid (135 mg, 0.14 mmol) and DIPEA (738.44 mg, 5.72 mmol) in CH₂Cl₂ (13.5 mL) at 0° C., then was added HOBT (193.01 mg, 1.43 mmol) and EDCl (821.44 mg, 4.20 mmol) at 0° C. The resulting mixture was stirred for 2 h at room temperature. The reaction was quenched with H₂O at 0° C. and extracted with EtOAc (3×100 mL). The combined organic layers were washed with H₂O (3×10 mL), dried over anhydrous Na₂SO₄. After filtration, the filtrate was concentrated under reduced pressure. The residue was purified by Prep-TLC (PE: EtOAc=1:3) to afford tert-butyl ((6³S,3S,4S,Z)-3-((S)-3-cyanomorpholino)-1²-(2-((S)-1-methoxyethyl)pyridin-3-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (42 mg, 31.0% yield) as an off-white solid. LCMS (ESI): m/z [M+H] calc'd for C₄₉H₆₄N₈O₉S 941.45, found 941.4.

### Step 12

Into a 40 mL vial were added tert-butyl ((6³S,3S,4S,Z)-3-((S)-3-cyanomorpholino)-1²-(2-((S)-1-methoxyethyl)pyridin-3-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (36 mg, 0.04 mmol), CH₂Cl₂ (3.6 mL) and TFA (0.36 mL) at 0° C. The resulting mixture was stirred for 1 h at room temperature. The mixture was basified to pH 8 with saturated NaHCO₃(aq.) and extracted with EtOAc (3×15 mL). The combined organic layers were dried over anhydrous Na₂SO₄. After filtration, the filtrate was concentrated under reduced pressure to afford (3S)-4-((6³S,3S,4S,Z)-4-amino-1²-(2-((S)-1-methoxyethyl)pyridin-3-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-3-yl) morpholine-3-carbonitrile (47 mg, crude) as an yellow oil. LCMS (ESI): m/z [M+H] calc'd for C₄₄H₅₆N₈O₇S 841.40, found 421.3 [M/2+H]+.

### Step 13

Into a 40 mL vial were added (3S)-4-((6³S,3S,4S,Z)-4-amino-1²-(2-((S)-1-methoxyethyl)pyridin-3-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-3-yl) morpholine-3-carbonitrile (47 mg, 0.06 mmol) and CH₂Cl₂ (2 mL) at 0° C. DIPEA (72.23 mg, 0.56 mmol), (1R,2S,3R)-2-methyl-3-(pyrimidin-4-yl) cyclopropane-1-carboxylic acid (29.87 mg, 0.07 mmol) and HATU (31.87 mg, 0.08 mmol) was then added. The resulting mixture was stirred for 2 h at room temperature. The reaction was quenched with H₂O at 0° C. and extracted with EtOAc (3×100 mL) and dried over anhydrous Na₂SO₄. After filtration, the filtrate was concentrated under reduced pressure. The residue was purified by Prep-TLC (EtOAc) to afford crude product as a light yellow solid. The crude product (20 mg) was purified by Prep-HPLC to afford (1R,2S,3R)—N-((6³S,3S,4S,Z)-3-((S)-3-cyanomorpholino)-1²-(2-((S)-1-methoxyethyl)pyridin-3-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methyl-3-(pyrimidin-4-yl) cyclopropane-1-carboxamide (7 mg, 12.0%) as a white solid. LCMS (ESI): m/z [M+H] calc'd for C₅₃H₆₄N₁₀O₈S 1001.46, found 1001.4. ¹H NMR (400 MHZ, DMSO-d₆) δ 9.13 (d, J=1.3 Hz, 1H), 8.74 (dd, J=4.7, 1.8 Hz, 1H), 8.69 (d, J=5.3 Hz, 1H), 8.49-8.39 (m, 2H), 7.92 (s, 1H), 7.77 (d, J=7.7 Hz, 1H), 7.73-7.64 (m, 2H), 7.61 (d, J=8.6 Hz, 1H), 7.52 (dd, J=7.7, 4.7 Hz, 1H), 6.10-6.03 (m, 1H), 5.23 (d, J=12.2 Hz, 1H), 4.64 (s, 1H), 4.44 (d, J=14.8 Hz, 1H), 4.27-4.17 (m, 2H), 4.07 (d, J=12.4 Hz, 1H), 3.98 (d, J=11.7 Hz, 1H), 3.88 (s, 1H), 3.71 (t, J=9.4 Hz, 2H), 3.60-3.50 (m, 1H), 3.43 (s, 1H), 3.23 (s, 3H), 3.18-3.11 (m, 1H), 3.12 (s, 3H), 2.98 (s, 1H), 2.78 (s, 1H), 2.65 (dd, J=9.7, 4.6 Hz, 1H), 2.35 (s, 1H), 1.99 (s, 1H), 1.77 (dd, J=15.7, 5.9 Hz, 1H), 1.77 (s, 2H), 1.48 (d, J=15.6 Hz, 4H), 1.37 (d, J=6.1 Hz, 3H), 1.24 (s, 1H), 1.15-1.02 (m, 4H), 0.89 (s, 3H), 0.35 (s, 3H).

### Example 7: Synthesis of (1r,2R,3S)—N-((6⁴S,4S,Z)-1¹-(2-(((S)-2,2-dimethyltetrahydro-2H-pyran-4-yl)oxy)ethyl)-1₂-(2-((S)-1-methoxyethyl)pyridin-3-yl)-10,10-dimethyl-5,7-dioxo-1¹H-8-oxa-6₂,6₃-diaza-2(4,2)-oxazola-1(5,3)-indola-6 (2,4)-bicyclo[3.1.1]heptanacycloundecaphane-4-yl)-2,3-dimethylcyclopropane-1-carboxamide

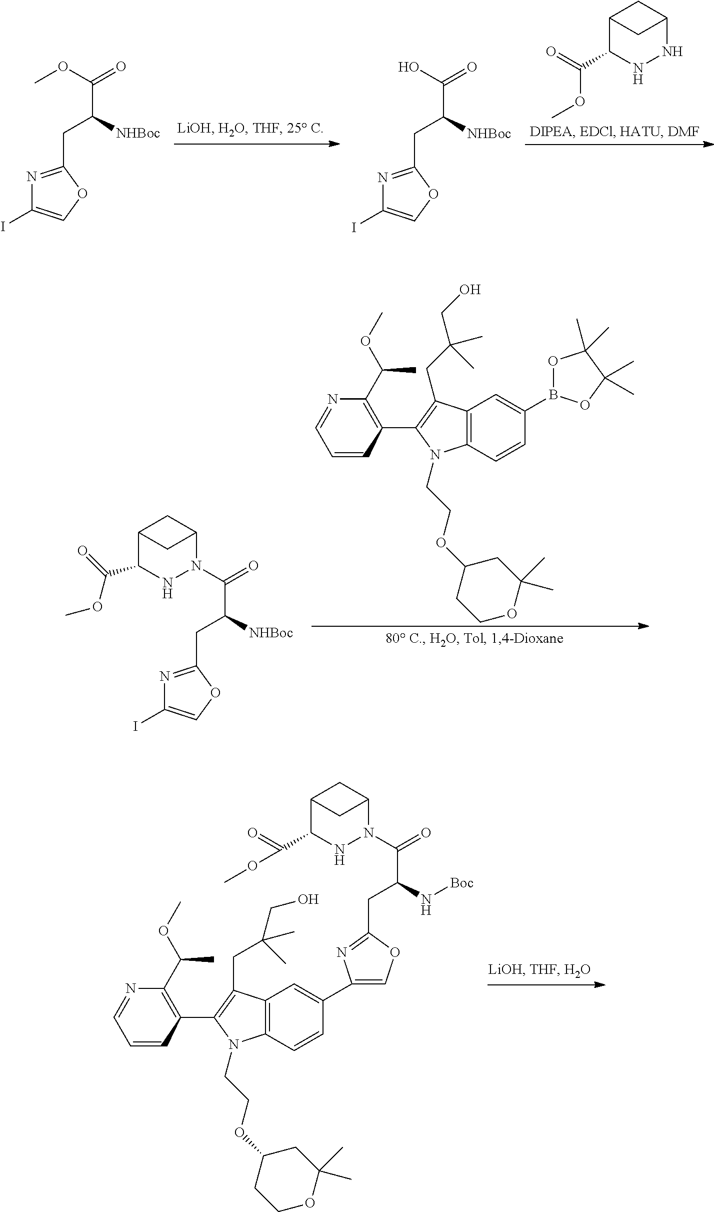

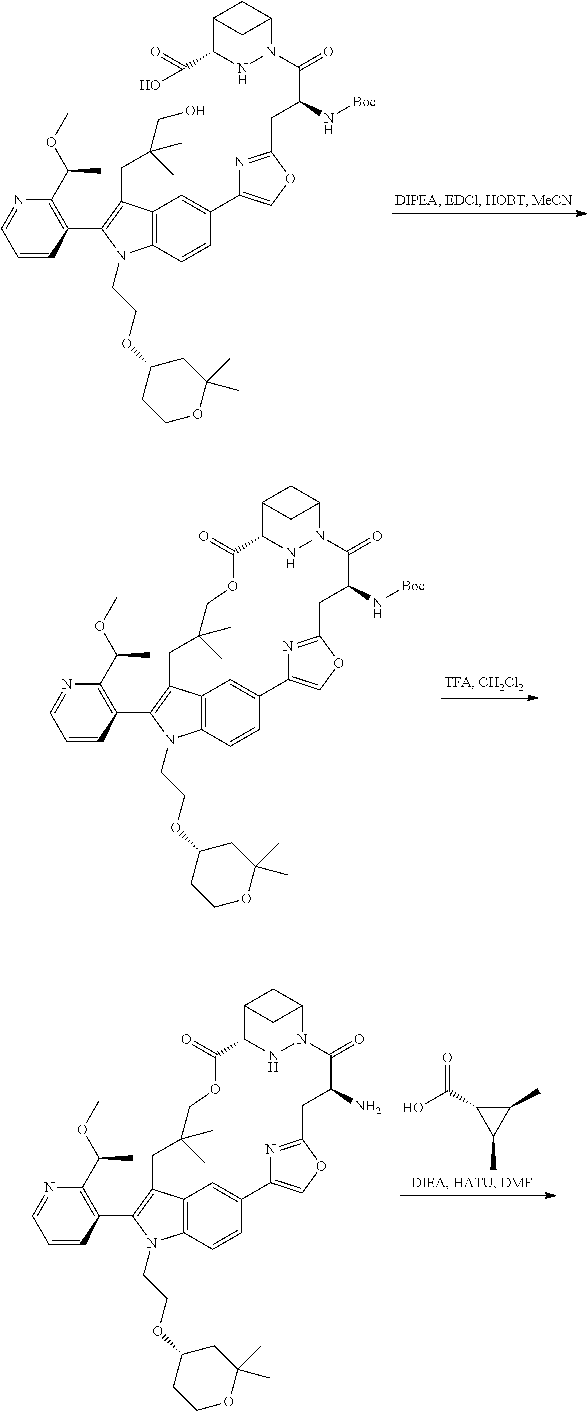

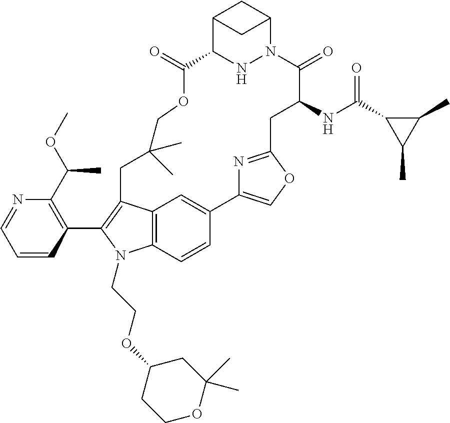

### Step 1

To a stirred solution of methyl(S)-2-((tert-butoxycarbonyl)amino)-3-(4-iodooxazol-2-yl) propanoate (5.8 g, 14.64 mmol) in THF (50 ml) and H₂O (50 mL) was added LiOH·H₂O (2.46 g, 58.56 mmol) in portions at 0° C. under an air atmosphere. The final reaction mixture was stirred for 2 h at 25° C. Desired product could be detected by LCMS. The residue was neutralized to pH 7 with citric acid. The resulting mixture was concentrated under reduced pressure. The residue was acidified to pH 5 with citric acid. The aqueous layer was extracted with CH₂Cl₂ (3×100 mL). Then organic phase was concentrated under reduced pressure. This resulted in(S)-2-((tert-butoxycarbonyl)amino)-3-(4-iodooxazol-2-yl) propanoic acid (5 g, crude) as a white solid. LCMS (ESI): m/z [M+H] calc'd for C₁₁H₁₅IN₂O₅ 383.00, found 383.0.

### Step 2

A solution of methyl(S)-2,3-diazabicyclo[3.1.1]heptane-4-carboxylate (0.12 g, 0.79 mmol) in DMF (10 ml) was treated with DIPEA (0.34 g, 2.62 mmol) followed by the addition of(S)-2-((tert-butoxycarbonyl)amino)-3-(4-iodooxazol-2-yl) propanoic acid (100 mg, 0.26 mmol) at 0° C. To the above mixture was added HATU (0.20 g, 0.52 mmol) at 0° C. The resulting mixture was stirred for additional 2 h at 25° C. Desired product could be detected by LCMS. The resulting mixture was diluted with EtOAc (40 mL). The resulting mixture was washed with H₂O (3×30 mL). The resulting mixture was concentrated under reduced pressure. The residue was purified by silica gel column chromatography, eluted with PE/EtOAc (3:1) to afford methyl(S)-2-((S)-2-((tert-butoxycarbonyl)amino)-3-(4-iodooxazol-2-yl) propanoyl)-2,3-diazabicyclo[3.1.1]heptane-4-carboxylate (3.2 g, 79.22%) as a brown solid. LCMS (ESI): m/z [M+H] calc'd for C₁₈H₂₅IN₄O₆ 521.08, found 521.05.

### Step 3

To a stirred solution of 3-(1-(2-(((S)-2,2-dimethyltetrahydro-2H-pyran-4-yl)oxy)ethyl)-2-(2-((S)-1-methoxyethyl)pyridin-3-yl)-5-(4,4,5,5-tetramethyl-1,3,2-dioxaborolan-2-yl)-1H-indol-3-yl)-2,2-dimethylpropan-1-ol (0.84 g, 1.77 mmol) and methyl(S)-2-((S)-2-((tert-butoxycarbonyl)amino)-3-(4-iodooxazol-2-yl) propanoyl)-2,3-diazabicyclo[3.1.1]heptane-4-carboxylate (0.84 g, 1.77 mmol) in toluene (9 ml), 1,4-dioxane (3 ml), H₂O (3 ml) were added K₃PO₄ (1.13 g, 5.32 mmol) and Pd(DtBPF)Cl₂ (0.69 g, 1.06 mmol) in portions at 25° C. under a nitrogen atmosphere. The final reaction mixture was stirred for 1 h at 70° C. Desired product could be detected by LCMS. The resulting mixture was diluted with H₂O (20 mL). The aqueous layer was extracted with EtOAc (3×50 mL). The resulting mixture was concentrated under reduced pressure. The residue was purified by silica gel column chromatography, eluted with PE/EtOAc (1:3) to afford methyl(S)-2-((S)-2-((tert-butoxycarbonyl)amino)-3-(4-(1-(2-(((S)-2,2-dimethyltetrahydro-2H-pyran-4-yl)oxy)ethyl)-3-(3-hydroxy-2,2-dimethylpropyl)-2-(2-((S)-1-methoxyethyl)pyridin-3-yl)-1H-indol-5-yl) oxazol-2-yl) propanoyl)-2,3-diazabicyclo[3.1.1]heptane-4-carboxylate (1.45 g, 78.39%) as a brown solid.

### Step 4

To a stirred mixture of methyl(S)-2-((S)-2-((tert-butoxycarbonyl)amino)-3-(4-(1-(2-(((S)-2,2-dimethyltetrahydro-2H-pyran-4-yl)oxy)ethyl)-3-(3-hydroxy-2,2-dimethylpropyl)-2-(2-((S)-1-methoxyethyl)pyridin-3-yl)-1H-indol-5-yl) oxazol-2-yl) propanoyl)-2,3-diazabicyclo[3.1.1]heptane-4-carboxylate (20 mg, 0.02 mmol) in THF (10 ml) and H₂O (10 mL) were added LiOH·H₂O (0.24 g, 5.64 mmol) in portions at 0° C. under an air atmosphere. The final reaction mixture was irradiated with microwave radiation for 2 h at 25° C. Desired product could be detected by LCMS. The mixture was neutralized to pH 7 with citric acid. The resulting mixture was concentrated under reduced pressure. The mixture was acidified to pH 5 with citric acid. The resulting mixture was extracted with CH₂Cl₂ (3×20 mL). The combined organic layers were dried over anhydrous Na₂SO₄. After filtration, the filtrate was concentrated under reduced pressure. This resulted in(S)-2-((S)-2-((tert-butoxycarbonyl)amino)-3-(4-(1-(2-(((S)-2,2-dimethyltetrahydro-2H-pyran-4-yl)oxy)ethyl)-3-(3-hydroxy-2,2-dimethylpropyl)-2-(2-((S)-1-methoxyethyl)pyridin-3-yl)-1H-indol-5-yl) oxazol-2-yl) propanoyl)-2,3-diazabicyclo[3.1.1]heptane-4-carboxylic acid (1 g, crude) as a brown solid. LCMS (ESI): m/z [M+H] calc'd for C₄₇H₆₄N₆O₁₀ 872.47, found 437.55 [M/2+H]⁺.

### Step 5

To a stirred solution of(S)-2-((S)-2-((tert-butoxycarbonyl)amino)-3-(4-(1-(2-(((S)-2,2-dimethyltetrahydro-2H-pyran-4-yl)oxy)ethyl)-3-(3-hydroxy-2,2-dimethylpropyl)-2-(2-((S)-1-methoxyethyl)pyridin-3-yl)-1H-indol-5-yl) oxazol-2-yl) propanoyl)-2,3-diazabicyclo[3.1.1]heptane-4-carboxylic acid (1 g, 1.15 mmol) and DIPEA (5181.36 mg, 40.08 mmol) in MeCN (100 ml) were added EDCl (7685.03 mg, 40.08 mmol) and HOBT (1547.73 mg, 11.45 mmol) dropwise at 0° C. under an air atmosphere. The final reaction mixture was stirred for 2 h at 35° C. Desired product could be detected by LCMS. The resulting mixture was diluted with EtOAc (500 mL) and washed with H₂O (3×300 mL). The resulting mixture was concentrated under reduced pressure. The residue was purified by silica gel column chromatography, eluted with PE/EtOAc (3:1) to afford tert-butyl ((6⁴S,4S,Z)-1¹-(2-(((S)-2,2-dimethyltetrahydro-2H-pyran-4-yl)oxy)ethyl)-1²-(2-((S)-1-methoxyethyl)pyridin-3-yl)-10,10-dimethyl-5,7-dioxo-1¹H-8-oxa-6²,6³-diaza-2(4,2)-oxazola-1(5,3)-indola-6 (2,4)-bicyclo[3.1.1]heptanacycloundecaphane-4-yl) carbamate (300 mg, 30.63%) as a brown solid. LCMS (ESI): m/z [M+H] calc'd for C₄₇H₆₂N₆O₉ 855.46, found 855.45.

### Step 6

Into a round-bottom flask were added tert-butyl ((6⁴S,4S,Z)-1¹-(2-(((S)-2,2-dimethyltetrahydro-2H-pyran-4-yl)oxy)ethyl)-1²-(2-((S)-1-methoxyethyl)pyridin-3-yl)-10,10-dimethyl-5,7-dioxo-1¹H-8-oxa-6²,6³-diaza-2(4,2)-oxazola-1(5,3)-indola-6 (2,4)-bicyclo[3.1.1]heptanacycloundecaphane-4-yl) carbamate (430 mg, 0.50 mmol) and CH₂Cl₂ (25 mL) at 0° C. To the above mixture was added TFA (5 mL, 67.32 mmol) dropwise over 5 min at 0° C. The resulting mixture was stirred for additional 0.5 h at 25° C. Desired product could be detected by LCMS. The reaction was quenched by the addition of sat. NaHCO₃(aq.) (30 mL) at 0° C. The aqueous layer was extracted with CH₂Cl₂ (3×40 mL). The resulting mixture was concentrated under reduced pressure. This resulted in (6⁴S,4S,Z)-4-amino-1¹-(2-(((S)-2,2-dimethyltetrahydro-2H-pyran-4-yl)oxy)ethyl)-1²-(2-((S)-1-methoxyethyl)pyridin-3-yl)-10,10-dimethyl-1¹H-8-oxa-6²,6³-diaza-2(4,2)-oxazola-1(5,3)-indola-6 (2,4)-bicyclo[3.1.1]heptanacycloundecaphane-5,7-dione (800 mg, crude) as a yellow solid. LCMS (ESI): m/z [M+H] calc'd for C₄₂H₅₄N₆O₇ 755.41, found 378.30. [M/2+H]⁺;

### Step 7

A solution of (1R,2R,3S)-2,3-dimethylcyclopropane-1-carboxylic acid (60.48 mg, 0.53 mmol) in DMF (1 ml) was treated with DIPEA (171.20 mg, 1.33 mmol) for 5 min at 0° C. under an air atmosphere. To the above mixture was added (6⁴S,4S,Z)-4-amino-1¹-(2-(((S)-2,2-dimethyltetrahydro-2H-pyran-4-yl)oxy)ethyl)-1²-(2-((S)-1-methoxyethyl)pyridin-3-yl)-10,10-dimethyl-1¹H-8-oxa-6²,6³-diaza-2(4,2)-oxazola-1(5,3)-indola-6 (2,4)-bicyclo[3.1.1]heptanacycloundecaphane-5,7-dione (200 mg, 0.27 mmol) in portions at 0° C. The resulting mixture was stirred for additional 5 min at 0° C. To the above mixture was added HATU (120.88 mg, 0.32 mmol) dropwise at 0° C. The resulting mixture was stirred for additional 1 h at 25° C. Desired product could be detected by LCMS. The crude product (80 mg) was purified by Prep-HPLC to afford (1r,2R,3S)—N-((6⁴S,4S,Z)-1¹-(2-(((S)-2,2-dimethyltetrahydro-2H-pyran-4-yl)oxy)ethyl)-1²-(2-((S)-1-methoxyethyl)pyridin-3-yl)-10,10-dimethyl-5,7-dioxo-1¹H-8-oxa-6²,6³-diaza-2(4,2)-oxazola-1(5,3)-indola-6(2,4)-bicyclo[3.1.1]heptanacycloundecaphane-4-yl)-2,3-dimethylcyclopropane-1-carboxamide (4.9 mg, 2.14%) as a white solid. LCMS (ESI): m/z [M+H] calc'd for C₄₈H₆₂N₆O₈ 851.46, found 851.4. ¹H NMR (400 MHz, DMSO-d6) δ 8.74-8.6 (m, 1H), 8.45-8.4 (m, 1H), 8.35 (s, 1H), 8.16-8.1 (m, 1H), 7.79-7.70 (m, 1H), 7.67-7.52 (m, 1H), 7.51-7.30 (m, 2H), 5.99-5.72 (m, 1H), 5.70-5.22 (m, 1H), 4.91-4.61 (m, 1H), 4.51-4.32 (m, 2H), 4.27-4.02 (m, 2H), 3.62-3.45 (m, 4H), 3.32-3.25 (m, 2H), 3.32-3.15 (m, 4H), 3.10-2.95 (m, 2H), 2.91-2.80 (m, 1H), 2.62-2.52 (m, 1H), 2.45-2.40 (m, 1H), 2.32-2.20 (m, 1H), 2.15-2.01 (m, 1H), 1.55-1.40 (m, 3H), 1.40-1.30 (m, 3H), 1.30-1.20 (m, 1H), 1.20-1.15 (m, 2H), 1.10-1.01 (m, 7H), 1.00-0.99 (m, 3H), 0.99-0.92 (m, 4H), 0.92-0.80 (m, 4H), 0.35-0.21 (m, 3H).

### Intermediate 7. Synthesis of 3-oxaspiro[bicyclo[2.1.1]hexane-2,1′-cyclopropane]-4-carboxylic acid

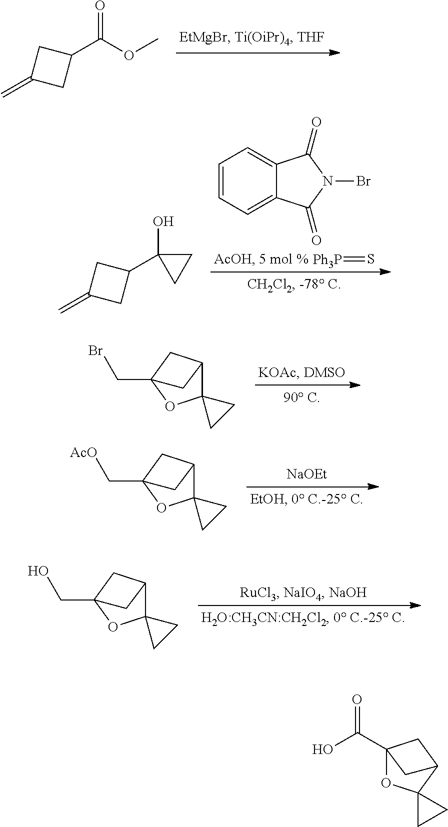

### Step 1

To a stirred solution of methyl 3-methylenecyclobutane-1-carboxylate (20 g, 158.535 mmol) in THF (300 mL) was added tetrakis(propan-2-yloxy) titanium (9.01 g, 31.707 mmol) for 5 min at 0° C. under a nitrogen atmosphere followed by the addition of bromo (ethyl) magnesium (42.26 g, 317.070 mmol) dropwise at 0° C. The resulting mixture was stirred for 2 h at 25° C. under a nitrogen atmosphere. The reaction was quenched with sat. aq. NH₄Cl at 0° C. The resulting mixture was extracted with MTBE (3×300 mL). The combined organic layers were washed with H₂O (1×500 mL), dried over anhydrous Na₂SO₄. After filtration, the filtrate was concentrated under reduced pressure. The residue was purified by silica gel column chromatography, eluted with pentane/MTBE (4:1) to afford 1-(3-methylenecyclobutyl)cyclopropan-1-ol (13.5 g, 68.57%) as a yellow solid. LCMS (ESI): m/z [M+H] calc'd for C₈H₁₂O 124.1; found 124.1.

### Step 2

To an oven-dried 100-mL round-bottom flask was added N-bromophthalimide (28.39 g, 125.621 mmol) dissolved in dry CH₂Cl₂ (900 mL) under N₂. The reaction vessel was cooled to −78° C. in a dry ice/acetone bath prior to the addition of a solution of 1-(3-methylenecyclobutyl)cyclopropan-1-ol (13 g, 104.684 mmol) and acetic acid (6.29 g, 104.684 mmol) in dry CH₂Cl₂ (10 mL). Then, the triphenylphosphine sulfide (1.54 g, 5.234 mmol) solution was added. The reaction was stirred for 2.5 h at −78° C. The reaction was quenched with a mixture of sat. aq. sodium thiosulfate solution (500 mL) and sat. aq. sodium bicarbonate solution (500 mL). The mixture was diluted with H₂O (800 mL) and transferred to a separatory funnel. The aqueous phase was extracted with EtOAc (2×1000 mL). The combined organic phases were dried over sodium sulfate and concentrated under reduced pressure into a white crude paste. The residue was purified by silica gel column chromatography, eluted with PE/EtOAc (8:1) to afford 4-(bromomethyl)-3-oxaspiro[bicyclo[2.1.1]hexane-2,1′-cyclopropane](8 g, 37.63%) as a colorless oil. LCMS (ESI): m/z [M+H] calc'd for C₈H₁₁BrO 202.0; found 202.0.

### Step 3

A mixture of 4-(bromomethyl)-3-oxaspiro[bicyclo[2.1.1]hexane-2,1′-cyclopropane](8 g, 39.394 mmol) and potassium acetate (12.37 g, 126.061 mmol) in DMSO (30 mL) was stirred for 2 h at 90° C. under air atmosphere. The reaction was quenched with H₂O at 0° C. The resulting mixture was extracted with EtOAc (2×50 mL). The combined organic layers were washed with H₂O (3×80 mL), dried over anhydrous Na₂SO₄. After filtration, the filtrate was concentrated under reduced pressure. This resulted in (3-oxaspiro[bicyclo[2.1.1]hexane-2,1′-cyclopropan]-4-yl)methyl acetate (7 g, 97.52%) as a colorless oil. LCMS (ESI): m/z [M+H] calc'd for C₁₀H₁₄O₃ 182.1; found 182.1.

### Step 4

To a stirred mixture of (3-oxaspiro[bicyclo[2.1.1]hexane-2,1′-cyclopropan]-4-yl)methyl acetate (7 g, 38.415 mmol) in ethanol (140 mL) was added sodium ethoxide (3.92 g, 57.623 mmol) in portions at 0° C. under air atmosphere. The resulting mixture was stirred for 2 h at 25° C. under air atmosphere. The resulting mixture was concentrated under reduced pressure. The reaction was quenched with sat. aq. NH₄Cl at 0° C. The aqueous layer was extracted with EtOAc (3×50 mL). The resulting mixture was concentrated under reduced pressure. The residue was purified by silica gel column chromatography, eluted with PE/EtOAc (5:1) to afford (3-oxaspiro[bicyclo[2.1.1]hexane-2,1′-cyclopropan]-4-yl) methanol (5.3 g, 98.42%) as a yellow oil. LCMS (ESI): m/z [M+H] calc'd for C₈H₁₂O₂ 140.1; found 140.1.

### Step 5

To a stirred mixture of (3-oxaspiro[bicyclo[2.1.1]hexane-2,1′-cyclopropan]-4-yl) methanol (5 g, 35.668 mmol) in CH₂Cl₂ (20 mL)/MeCN (20 mL)/H₂O (30 mL) were added RuCl₃·H₂O (0.80 g, 3.567 mmol) and NaOH (5.71 g, 142.672 mmol) in portions at 0° C. under air atmosphere. To the above mixture was added NaIO₄ (22.89 g, 107.004 mmol) in portions over 5 min at 0° C. The resulting mixture was stirred for an additional 20 h at 25° C. The reaction was filtered to remove the ruthenium and diluted with 100 mL of H₂O. The aqueous phase was washed with MTBE (2×100 mL). The aqueous layer was acidified with 5 M HCl (5 mL) and extracted with EtOAc (4×100 mL). The resulting mixture was concentrated under reduced pressure. This resulted in 3-oxaspiro[bicyclo[2.1.1]hexane-2,1′-cyclopropane]-4-carboxylic acid (4.5 g, 81.84%) as a yellow oil. LCMS (ESI): m/z [M+H] calc'd for C₈H₁₀O₃ 154.1; found 154.1.

### Example 8: Synthesis of (1R,3S,4R)-3-methyl-N-((1⁵S,1⁸S,6³S,3S,4S,Z)-1⁵,1⁸,10,10-tetramethyl-5,7-dioxo-3-(2-oxa-6-azaspiro[3.3]heptan-6-yl)-1⁷,1⁸,1¹⁰,1¹¹,6¹,6²,6³,6⁴,6⁵,6⁶-decahydro-1⁵H-8-oxa-1 (15,17)-pyrido[3′,2′: 9,10][1,4]dioxa[7]azacycloundecino[7,8-a]indola-2(4,2)-thiazola-6(1,3)-pyridazinacycloundecaphane-4-yl)-2-oxabicyclo[2.1.1]hexane-1-carboxamide

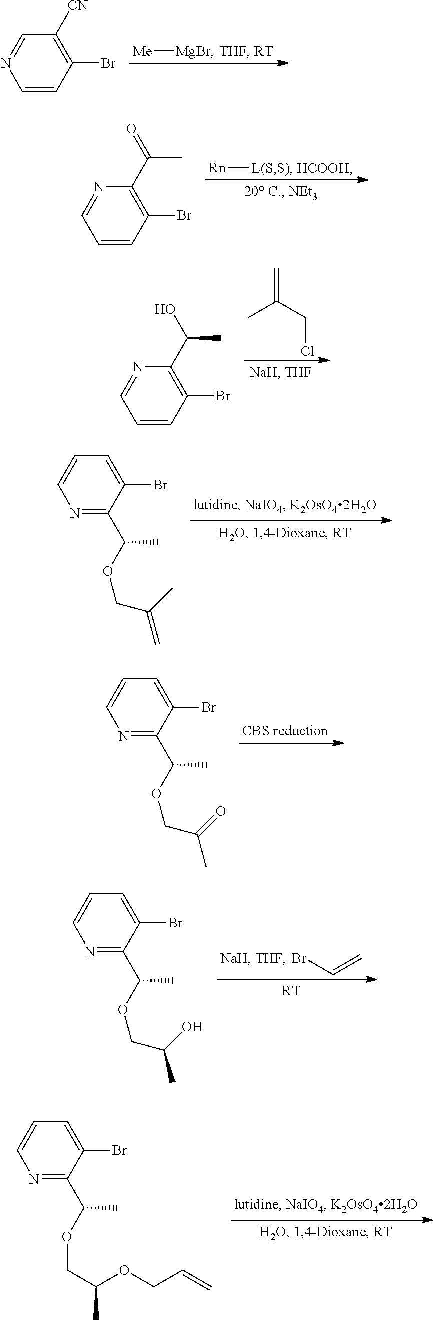

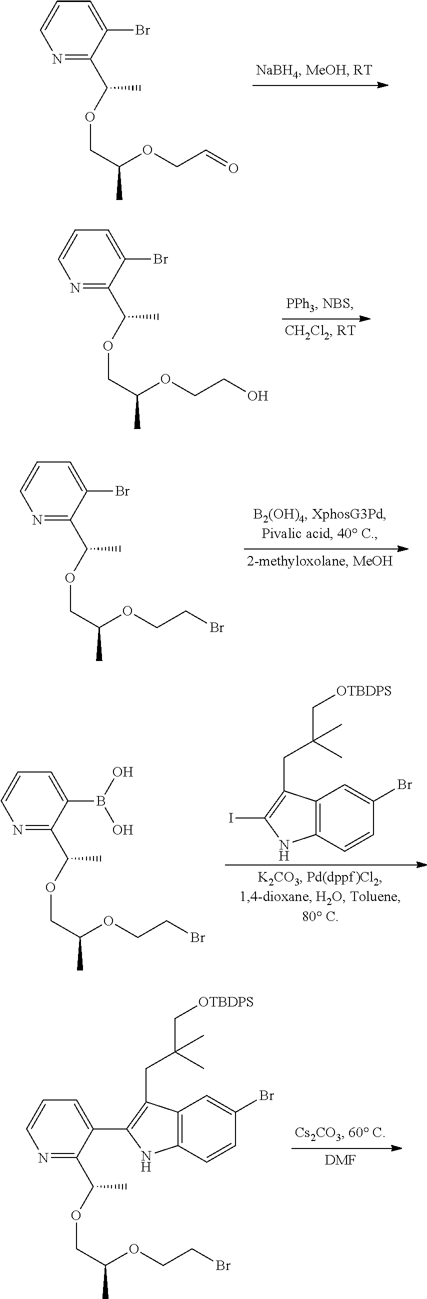

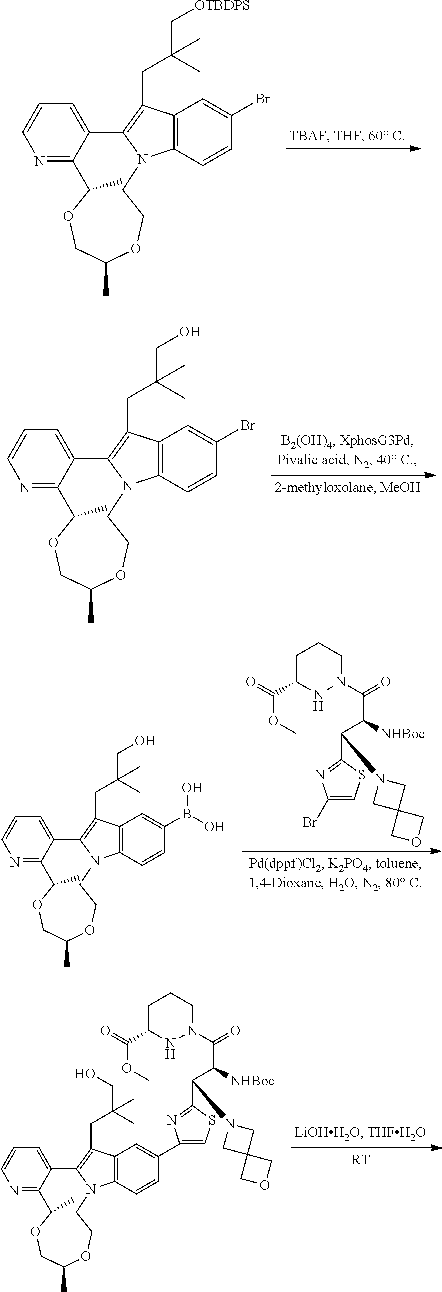

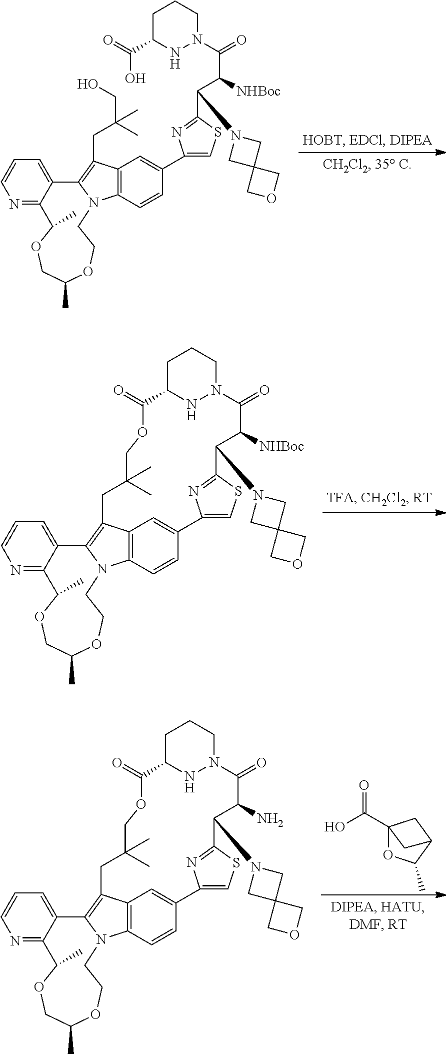

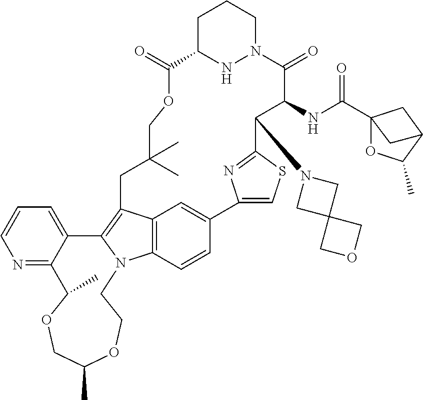 [SMILES: C[C@@H]1OC[C@H](C)OCCn2c(c3c4cc(ccc42)-c2csc(n2)[C@@H](N2CC4(COC4)C2)[C@H](NC(=O)C24CC(C2)[C@H](C)O4)C(=O)N2CCC[C@H](N2)C(=O)OCC(C)(C)C3)-c2cccnc21]

### Step 1

To a stirred solution of 3-bromopicolinonitrile (20 g) in THF (180 mL) were added Me-MgBr (40.29 mL) dropwise at 0° C. under air atmosphere. The resulting mixture was stirred for 2 h at 10° C. under a nitrogen atmosphere. The reaction was quenched by the addition of sat. NH₄Cl (aq.) at 0° C. The mixture was neutralized to pH 6 with saturated NaHCO₃(aq.). The resulting mixture was extracted with EtOAc (1×200 mL). The combined organic layers were washed with H₂O (3×200 mL), dried over anhydrous Na₂SO₄. After filtration, the filtrate was concentrated under reduced pressure. The crude product mixture was used in the next step directly without further purification. LCMS (ESI): m/z [M+H] calc'd for C₇He₆rNO 199.96, found 200.00.

### Step 2

A solution of NEt₃ (28.27 g, 279.40 mmol) in HCOOH (11.43 g) was treated with (S,S)—N-(p-Toluenesulfonyl)-1,2-diphenylethanediamine (chloro) (p-cymene) ruthenium (II) (0.33 g) for 10 min at 10° C. under an argon atmosphere. The reaction mixture was followed by the addition of 1-(3-bromopyridin-2-yl) ethan-1-one (20.7 g) dropwise at 10° C. The resulting mixture was stirred for 2 h at 25° C. under argon atmosphere. The reaction was quenched by the addition of sat. NH₄Cl (aq.) at 0° C. The resulting mixture was extracted with EtOAc (3×40 mL). The combined organic layers were washed with H₂O (3×100 mL), dried over anhydrous Na₂SO₄. After filtration, the filtrate was concentrated under reduced pressure. This resulted in(S)-1-(3-bromopyridin-2-yl) ethan-1-ol (21 g, 75.33%) as a black oil. ESI-MS m/z=201.95 [M+H]⁺; Calculated MW: 200.98. LCMS (ESI): m/z [M+H] calc'd for C₇H₉BrNO 201.98, found 201.95.

### Step 3

A solution of(S)-1-(3-bromopyridin-2-yl) ethan-1-ol (1 g) in THF (10 mL) was treated with NaH (178.16 mg) for 30 min at 0° C. under an air atmosphere followed by the addition of 3-chloro-2-methylprop-1-ene (1344.46 mg) dropwise at 0° C. The resulting mixture was stirred for 2 h at 25° C. The reaction was quenched by the addition of H₂O (15 mL) at 0° C. The resulting mixture was extracted with EtOAc (3×20 mL). The combined organic layers were dried over anhydrous Na₂SO₄. After filtration, the filtrate was concentrated under reduced pressure. The residue was purified by Prep-TLC (PE/EtOAc 1:2) to(S)-3-bromo-2-(1-((2-methylallyl)oxy)ethyl)pyridine (980 mg, 61.84%) as a yellow oil. LCMS (ESI): m/z [M+H] calc'd for C₁₁H₁₄BrNO 256.03, found 256.0.

### Step 4

To a stirred solution of(S)-3-bromo-2-(1-((2-methylallyl)oxy)ethyl)pyridine (950 mg) in 1,4-dioxane (5 mL) and H₂O (5 mL) were added 2,6-lutidine (794.85 mg) and NaIO₄ (3173.16 mg) and K₂OsO₄.2H₂O (68.33 mg) dropwise at 0° C. under an air atmosphere. The resulting mixture was stirred for 2 h at 25° C. The resulting mixture was filtered, the filter cake was washed with EtOAc (2×10 mL). The filtrate was concentrated under reduced pressure. The resulting mixture was diluted with H₂O (10 mL). The resulting mixture was extracted with EtOAc (3×15 mL). The combined organic layers were dried over anhydrous Na₂SO₄. After filtration, the filtrate was concentrated under reduced pressure. This resulted in (S)-1-(1-(3-bromopyridin-2-yl) ethoxy) propan-2-one (850 mg, 71.03%) as a red oil. LCMS (ESI): m/z [M+H] calc'd for C₁₀H₁₂BrNO₂ 258.01, found 257.95.

### Step 5

To a stirred solution of NEt₃ (4.75 g) was added HCOOH (7.41 g) and (S,S)—N-(p-Toluenesulfonyl)-1,2-diphenylethanediamine (chloro) (p-cymene) ruthenium (II) (211.79 mg) in portions at 0° C. under an air atmosphere. The resulting mixture was stirred for 15 min at 40° C. under an argon atmosphere. To the above mixture was(S)-1-(1-(3-bromopyridin-2-yl) ethoxy) propan-2-one (13.56 g) and NEt₃ (4.75 g) dropwise over 5 min at 0° C. The resulting mixture was stirred for additional 2 h at 40° C. Desired product could be detected by LCMS. The resulting mixture was diluted with EtOAc (50 mL). The resulting mixture was washed with H₂O (2×50 mL). The combined organic layers were dried over anhydrous Na₂SO₄. After filtration, the filtrate was concentrated under reduced pressure. The crude product (12.3 g) was purified by Prep-HPLC to afford(S)-1-((S)-1-(3-bromopyridin-2-yl) ethoxy) propan-2-ol (9.2 g, 50.45%) as a yellow oil. LCMS (ESI): m/z [M+H] calc'd for C₁₀H₁₄BrNO₂ 260.02, found 259.95.

### Step 6

A solution of(S)-1-((S)-1-(3-bromopyridin-2-yl) ethoxy) propan-2-ol (9.15 g) in THF (100 mL) was treated with NaH (1.69 g) for 30 min at 30° C. under an air atmosphere followed by the addition of allyl bromide (17.02 g) in portions at 0° C. The resulting mixture was stirred for 2 h at 25° C. under an air atmosphere. The reaction was quenched by the addition of sat. NH₄Cl (aq.) (200 mL) at 0° C. The aqueous layer was extracted with EtOAc (3×200 mL). The residue was purified by silica gel column chromatography, eluted with PE/EtOAc (4:1) to afford 2-((S)-1-((S)-2-(allyloxy) propoxy)ethyl)-3-bromopyridine (12.4 g, 82.20%) as a yellow oil. LCMS (ESI): m/z [M+H] calc'd for C₁₃H₁₈BrNO₂ 300.05, found 299.95.

### Step 7

A solution of 2-((S)-1-((S)-2-(allyloxy) propoxy)ethyl)-3-bromopyridine (12.3 g) in 1,4-dioxane (250 mL) and H₂O (250 mL) was treated with 2,6-lutidine (8.78 g) at 0° C. under an air atmosphere followed by the addition of NaIO₄ (35.06 g) and K₂OsO₄·2H₂O (0.75 g) in portions at 0° C. The resulting mixture was stirred for 2 h at 25° C. The resulting mixture was filtered, the filter cake was washed with EtOAc (6×150 mL). The aqueous layer was extracted with EtOAc (6×300 mL). The combined organic layers were dried over anhydrous Na₂SO₄. After filtration, the filtrate was concentrated under reduced pressure. This resulted in 2-(((S)-1-((S)-1-(3-bromopyridin-2-yl) ethoxy) propan-2-yl)oxy) acetaldehyde (14.2 g, 86.02%) as a black oil. LCMS (ESI): m/z [M+H] calc'd for C₁₂H₁₆BrNO₃ 302.03, found 301.90.

### Step 8

A solution 2-(((S)-1-((S)-1-(3-bromopyridin-2-yl) ethoxy) propan-2-yl)oxy) acetaldehyde (10 g) in MeOH (140 mL) was treated with NaBH₄ (5.01 g) at 0° C. under air atmosphere. The resulting mixture was stirred for 2 h at 25° C. under an air atmosphere. The reaction was quenched by the addition of sat. NH 4Cl (aq.) (200 mL) at 0° C. The aqueous layer was extracted with EtOAc (3×200 mL). The combined organic layers were dried over anhydrous Na₂SO₄. After filtration, the filtrate was concentrated under reduced pressure. The residue was purified by silica gel column chromatography, eluted with PE/EtOAc (1:1) to afford 2-(((S)-1-((S)-1-(3-bromopyridin-2-yl) ethoxy) propan-2-yl)oxy)ethan-1-ol (4.43 g, 44.01%) as a yellow oil. LCMS (ESI): m/z [M+H] calc'd for C₁₂H₁₈BrNO 304.05, found 303.95.

### Step 9

A solution of 2-(((S)-1-((S)-1-(3-bromopyridin-2-yl) ethoxy) propan-2-yl)oxy)ethan-1-ol (15 g) in CH₂Cl₂ (150 mL) was treated with PPh3 (19.40 g) at 0° C. under an air atmosphere followed by the addition of NBS (13.17 g) in portions at 0° C. The resulting mixture was stirred for 1 h at 25° C. The residue was washed with H₂O (3×100 mL). The combined organic layers were dried over anhydrous Na₂SO₄. After filtration, the filtrate was concentrated under reduced pressure. The residue was purified by silica gel column chromatography, eluted with PE/EtOAc (3:1) to afford 3-bromo-2-((S)-1-((S)-2-(2-bromoethoxy) propoxy)ethyl)pyridine (14.3 g, 79.00%) as a yellow oil. LCMS (ESI): m/z [M+H] calc'd for C₁₂H₁₇Br₂NO₂ 365.96, found 367.85.

### Step 10

A solution of 3-bromo-2-((S)-1-((S)-2-(2-bromoethoxy) propoxy)ethyl)pyridine (14.12 g), B₂(OH)₄ (4.14 g), methyl 2,2-dimethylpropanoate (8.94 g) and XPhos Pd G3 (0.98 g) in 2-methyloxolane (100 mL) and MeOH (50 mL) was stirred at 25° C. under a nitrogen atmosphere. The resulting mixture was stirred for 2 h at 40° C. The precipitated solids were collected by filtration and washed with EtOAc (3×50 mL). The aqueous layer was extracted with EtOAc (3×100 mL). The combined organic layers were dried over anhydrous Na₂SO₄. After filtration, the filtrate was concentrated under reduced pressure. This resulted in (2-((S)-1-((S)-2-(2-bromoethoxy) propoxy)ethyl)pyridin-3-yl) boronic acid (18.12 g, 79.46%) as a yellow solid. LCMS (ESI): m/z [M+H] calc'd for C₁₂H₁₉BBrNO₄ 332.06, found 332.15.

### Step 11

A solution of (2-((S)-1-((S)-2-(2-bromoethoxy) propoxy)ethyl)pyridin-3-yl) boronic acid (18 g), 5-bromo-3-(3-((tert-butyldiphenylsilyl)oxy)-2,2-dimethylpropyl)-2-iodo-1H-indole (38.56 g), Pd(dppf)Cl₂ (4.42 g) and K₃PO₄ (28.77 g) in toluene (30 mL), 1,4-dioxane (10 mL) and H₂O (10 mL) at was stirred at 25° C. under a nitrogen atmosphere. The resulting mixture was stirred for 1 h at 80° C. The aqueous layer was extracted with EtOAc (3×10 mL). The combined organic layers were dried over anhydrous Na₂SO₄. After filtration, the filtrate was concentrated under reduced pressure. The residue was purified by silica gel column chromatography, eluted with PE/EtOAc (1:1) to afford 5-bromo-2-(2-((S)-1-((S)-2-(2-bromoethoxy) propoxy)ethyl)pyridin-3-yl)-3-(3-((tert-butyldiphenylsilyl)oxy)-2,2-dimethylpropyl)-1H-indole (16.03 g, 36.65%) as a yellow solid. LCMS (ESI): m/z [M+H] calc'd for C₄₁H₅₀Br₂N₂O₃Si 805.20, found 805.15.

### Step 12

A solution of 5-bromo-2-(2-((S)-1-((S)-2-(2-bromoethoxy) propoxy)ethyl)pyridin-3-yl)-3-(3-((tert-butyldiphenylsilyl)oxy)-2,2-dimethylpropyl)-1H-indole (15.4 g) in DMF (160 mL) was treated with Cs₂CO₃ (31097.50 mg) at 0° C. under an air atmosphere. The resulting mixture was stirred for 1 h at 60° C. The mixture was allowed to cool down to 25° C. The precipitated solids were collected by filtration and washed with EtOAc (3×100 mL). The residue was washed with H₂O (3×100 mL). The combined organic layers were dried over anhydrous Na₂SO₄. After filtration, the filtrate was concentrated under reduced pressure. The residue was purified by silica gel column chromatography, eluted with PE/EtOAc (2:1) to afford (5S,8S)-15-bromo-17-(3-((tert-butyldiphenylsilyl)oxy)-2,2-dimethylpropyl)-5,8-dimethyl-7,8,10,11-tetrahydro-5H-pyrido[3′,2′:9,10][1,4]dioxa[7]azacycloundecino[7,8-a]indole (10.66 g, 76.94%) as a yellow solid. LCMS (ESI): m/z [M+H] calc'd for C₄₁H₄₉BrN₂O₃Si 725.27, found 725.60.

### Step 13

A solution of (5S,8S)-15-bromo-17-(3-((tert-butyldiphenylsilyl)oxy)-2,2-dimethylpropyl)-5,8-dimethyl-7,8,10,11-tetrahydro-5H-pyrido[3′,2′: 9,10][1,4]dioxa[7]azacycloundecino[7,8-a]indole (11.43 g) in THF (120 mL) was treated with TBAF (8.23 g) at 0° C. under an air atmosphere. The resulting mixture was stirred for 2 h at 60° C. The mixture was allowed to cool down to 25° C. The resulting mixture was diluted with EtOAc (200 mL). The residue was washed with H₂O (3×100 mL). The combined organic layers were dried over anhydrous Na₂SO₄. After filtration, the filtrate was concentrated under reduced pressure. The residue was purified by silica gel column chromatography, eluted with PE/EtOAc (1:1) to afford-((5S,8S)-15-bromo-5,8-dimethyl-7,8,10,11-tetrahydro-5H-pyrido[3′,2′:9,10][1,4]dioxa[7]azacycloundecino[7,8-a]indol-17-yl)-2,2-dimethylpropan-1-ol (7.21 g, 93.93%) as a yellow solid. LCMS (ESI): m/z [M+H] calc'd for C₂₅H₃₁BrN₂O₃ 487.15, found 487.15.

### Step 14

A solution of 3-((5S,8S)-15-bromo-5,8-dimethyl-7,8,10,11-tetrahydro-5H-pyrido[3′,2′:9,10][1,4]dioxa[7]azacycloundecino[7,8-a]indol-17-yl)-2,2-dimethylpropan-1-ol (7 g) in 2-methyloxolane (70 mL) and MeOH (35 mL) was treated with XPhos Pd G3 (364.68 mg) B₂(OH)₄ (1544.93 mg) and potassium 2,2-dimethylpropanoate (4027.40 mg) at 25° C. under a nitrogen atmosphere. The resulting mixture was stirred for 2 h at 40° C. The mixture was allowed to cool down to 25° C. The precipitated solids were collected by filtration and washed with H₂O (2×50 mL). The aqueous layer was extracted with EtOAc (3×100 mL). The combined organic layers were dried over anhydrous Na₂SO₄. After filtration, the filtrate was concentrated under reduced pressure. This resulted in ((5S,8S)-17-(3-hydroxy-2,2-dimethylpropyl)-5,8-dimethyl-7,8,10,11-tetrahydro-5H-pyrido[3′,2′:9,10][1,4]dioxa[7]azacycloundecino[7,8-a]indol-15-yl) boronic acid (8.12 g, 93.75%) as a black solid. LCMS (ESI): m/z [M+H] calc'd for C₂₅H₃₃BN₂O₅ 453.25, found 453.25.

### Step 15

A solution of ((5S,8S)-17-(3-hydroxy-2,2-dimethylpropyl)-5,8-dimethyl-7,8,10,11-tetrahydro-5H-pyrido[3′,2′:9,10][1,4]dioxa[7]azacycloundecino[7,8-a]indol-15-yl) boronic acid (1.3 g), methyl(S)-1-((2S,3S)-3-(4-bromothiazol-2-yl)-2-((tert-butoxycarbonyl)amino)-3-(2-oxa-6-azaspiro[3.3]heptan-6-yl) propanoyl) hexahydropyridazine-3-carboxylate (1.3 g) and Pd(dppf)Cl₂ (0.23 g) in toluene (9 mL), 1,4-dioxane (3 mL) and H₂O (3 mL) was treated with K₃PO₄ (1.53 g) at 25° C. under a nitrogen atmosphere. The resulting mixture was stirred for 1 h at 80° C. The aqueous layer was extracted with EtOAc (3×5 mL). The combined organic layers were dried over anhydrous Na₂SO₄. After filtration, the filtrate was concentrated under reduced pressure. The residue was purified by Prep-TLC (PE/EtOAc 1:5) to afford methyl(S)-1-((2S,3S)-2-((tert-butoxycarbonyl)amino)-3-(4-((5S,8S)-17-(3-hydroxy-2,2-dimethylpropyl)-5,8-dimethyl-7,8,10,11-tetrahydro-5H-pyrido[3′,2′: 9,10][1,4]dioxa[7]azacycloundecino[7,8-a]indol-15-yl) thiazol-2-yl)-3-(2-oxa-6-azaspiro[3.3]heptan-6-yl) propanoyl) hexahydropyridazine-3-carboxylate (1.6 g, 77.14%) as a yellow solid. LCMS (ESI): m/z [M+H] calc'd for C₄₇H₆₃N₇O₉S 902.44, found 902.45.

### Step 16

A solution of methyl(S)-1-((2S,3S)-2-((tert-butoxycarbonyl)amino)-3-(4-((5S,8S)-17-(3-hydroxy-2,2-dimethylpropyl)-5,8-dimethyl-7,8,10,11-tetrahydro-5H-pyrido[3′,2′: 9,10][1,4]dioxa[7]azacycloundecino[7,8-a]indol-15-yl) thiazol-2-yl)-3-(2-oxa-6-azaspiro[3.3]heptan-6-yl) propanoyl) hexahydropyridazine-3-carboxylate (910 mg) in THF (5 mL) and H₂O (5 mL) was treated with LiOH·H₂O (169.31 mg) at 0° C. under an air atmosphere. The resulting mixture was stirred for 1 h at 25° C. The resulting mixture was washed with EtOAc (3×20 mL). The combined organic layers were dried over anhydrous Na₂SO₄. After filtration, the filtrate was concentrated under reduced pressure. This resulted in(S)-1-((2S,3S)-2-((tert-butoxycarbonyl)amino)-3-(4-((5S,8S)-17-(3-hydroxy-2,2-dimethylpropyl)-5,8-dimethyl-7,8,10,11-tetrahydro-5H-pyrido[3′,2′:9,10][1,4]dioxa[7]azacycloundecino[7,8-a]indol-15-yl) thiazol-2-yl)-3-(2-oxa-6-azaspiro[3.3]heptan-6-yl) propanoyl) hexahydropyridazine-3-carboxylic acid (890 mg, 99.35%) as a yellow solid. LCMS (ESI): m/z [M+H] calc'd for C₄₆H₆₁N₇O₉S 888.43, found 888.40.

### Step 17

A solution of(S)-1-((2S,3S)-2-((tert-butoxycarbonyl)amino)-3-(4-((5S,8S)-17-(3-hydroxy-2,2-dimethylpropyl)-5,8-dimethyl-7,8,10,11-tetrahydro-5H-pyrido[3′,2′:9,10][1,4]dioxa[7]azacycloundecino[7,8-a]indol-15-yl) thiazol-2-yl)-3-(2-oxa-6-azaspiro[3.3]heptan-6-yl) propanoyl) hexahydropyridazine-3-carboxylic acid (890 mg) in CH₂Cl₂ (500 mL) was treated with DIPEA (5181.00 mg) at 25° C. under an air atmosphere followed by the addition of EDCl (5763.37 mg) and HOBT (1354.17 mg) in portions at 0° C. The resulting mixture was stirred for 16 h at 35° C. The residue was washed with acetic acid (1×200 mL). The residue was washed with pH=9.4 borate buffer (1×200 mL). The residue was washed with H₂O (1×200 mL). The combined organic layers were dried over anhydrous Na₂SO₄. After filtration, the filtrate was concentrated under reduced pressure. The residue was purified by Prep-TLC (PE/EtOAc 1:5) to afford tert-butyl ((1⁵S,1⁸S,6³S,3S,4S,Z)-1⁵,1⁸,10,10-tetramethyl-5,7-dioxo-3-(2-oxa-6-azaspiro[3.3]heptan-6-yl)-1⁷,1⁸,1¹⁰,1¹¹,6¹,6²,6³,6⁴,6⁵,6⁶-decahydro-1⁵H-8-oxa-1 (15,17)-pyrido[3′,2′:9,10][1,4]dioxa[7]azacycloundecino[7,8-a]indola-2(4,2)-thiazola-6(1,3)-pyridazinacycloundecaphane-4-yl) carbamate 490 mg, 56.20%) as a yellow solid. LCMS (ESI): m/z [M+H] calc'd for C₄₆H₅₉N₇O₈S 870.41, found 870.45.

### Step 18

A solution of tert-butyl ((1⁵S,1⁸S,6³S,3S,4S,Z)-1⁵,1⁸,10,10-tetramethyl-5,7-dioxo-3-(2-oxa-6-azaspiro[3.3]heptan-6-yl)-1⁷,1⁸, 1¹⁰, 1¹¹,6¹,6²,6³,6⁴,6⁵,6⁶-decahydro-1⁵H-8-oxa-1 (15,17)-pyrido[3′,2′:9,10][1,4]dioxa[7]azacycloundecino[7,8-a]indola-2(4,2)-thiazola-6(1,3)-pyridazinacycloundecaphane-4-yl) carbamate (410 mg) in CH₂Cl₂ (5 mL) was treated with ZnBr₂ (3183.43 mg) at 25° C. under an air atmosphere. The resulting mixture was stirred for 16 h at 25° C. The mixture was neutralized to pH 8 with saturated NaHCO₃(aq.). The precipitated solids were collected by filtration and washed with CH₂Cl₂ (2×10 mL). The aqueous layer was extracted with CH₂Cl₂ (3×10 mL). 1The combined organic layers were dried over anhydrous Na₂SO₄. After filtration, the filtrate was concentrated under reduced pressure. This resulted in (1⁵S,1⁸S,6³S,3S,4S,Z)-4-amino-1⁵,1⁸,10,10-tetramethyl-3-(2-oxa-6-azaspiro[3.3]heptan-6-yl)-1⁷,1⁸,1¹⁰,1¹¹,6¹,6²,6³,6⁴,6⁵,6⁶-decahydro-1⁵H-8-oxa-1 (15,17)-pyrido[3′,2′:9,10][1,4]dioxa[7]azacycloundecino[7,8-a]indola-2(4,2)-thiazola-6(1,3)-pyridazinacycloundecaphane-5,7-dione (370 mg, 90.76%) as a yellow solid. LCMS (ESI): m/z [M+H] calc'd for C₄₁H₅₁N₇O₆S 770.36, found 770.30.

### Step 19

A solution of(S)-3-methyl-2-oxabicyclo[2.1.1]hexane-1-carboxylic acid (20.31 mg) in DMF (1 mL) was treated with DIPEA (167.86 mg) for 10 min at 0° C. under an air atmosphere followed by the addition of (15S,18S,6³S,3S,4S,Z)-4-amino-1⁵,1⁸,10,10-tetramethyl-3-(2-oxa-6-azaspiro[3.3]heptan-6-yl)-1⁷,1⁸,1¹⁰,1¹¹,6¹,6²,6³,6⁴,6⁵,6⁶-decahydro-1⁵H-8-oxa-1 (15,17)-pyrido[3′,2′:9,10][1,4]dioxa[7]azacycloundecino[7,8-a]indola-2(4,2)-thiazola-6(1,3)-pyridazinacycloundecaphane-5,7-dione (100 mg) and HATU (98.77 mg) in portions at 0° C. The resulting mixture was stirred for 1 h at 25° C. The resulting mixture was diluted with EtOAc (3 mL). The residue was washed with H₂O (3×6 mL). The combined organic layers were dried over anhydrous Na₂SO₄. After filtration, the filtrate was concentrated under reduced pressure. The crude product (110 mg) was purified by Prep-HPLC to afford (1R,3S,4R)-3-methyl-N-((1⁵S,1⁸S,6³S,3S,4S,Z)-1⁵,1⁸,10,10-tetramethyl-5,7-dioxo-3-(2-oxa-6-azaspiro[3.3]heptan-6-yl)-1⁷,1⁸,1¹⁰, 1¹¹,6¹,6²,6³,6⁴,6⁵,6⁶-decahydro-1⁵H-8-oxa-1 (15,17)-pyrido[3′,2′:9,10][1,4]dioxa[7]azacycloundecino[7,8-a]indola-2(4,2)-thiazola-6(1,3)-pyridazinacycloundecaphane-4-yl)-2-oxabicyclo[2.1.1]hexane-1-carboxamide (51.1 mg, 43.12%) as a white solid. LCMS (ESI): m/z [M+H] calc'd for C₄₈H₅₉N₇₀₈S 894.41, found 893.41. ¹H NMR (400 MHZ, Methanol-d4) δ 8.65 (s, 2H), 7.79 (s, 1H), 7.69 (s, 1H), 7.55 (s, 1H), 7.45 (s, 2H), 5.96 (s, 1H), 4.72 (s, 4H), 4.58 (s, 1H), 4.46 (s, 1H), 4.41-4.37 (m, 4H), 4.04-3.89 (m, 3H), 3.71 (s, 2H), 3.66 (s, 2H), 3.56 (s, 3H), 3.38 (s, 1H), 3.29-3.12 (m, 2H), 2.75 (s, 1H), 2.67 (s, 1H), 2.31-2.98 (m, 1H), 2.20 (s, 1H), 2.03 (s, 1H), 1.88-1.71 (m, 4H), 1.58 (s, 4H), 1.31-1.26 (m, 4H), 0.90 (s, 3H), 0.81 (s, 3H), 0.54 (s, 3H)

### Example 9: Synthesis of (2R,3S)—N-((6³S,3S,4S,Z)-12-(5-(1-cyclopropylpiperidin-4-yl)-2-((S)-1-methoxyethyl)pyridin-3-yl)-3-(3-methoxy-3-methylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyloxetane-2-carboxamide

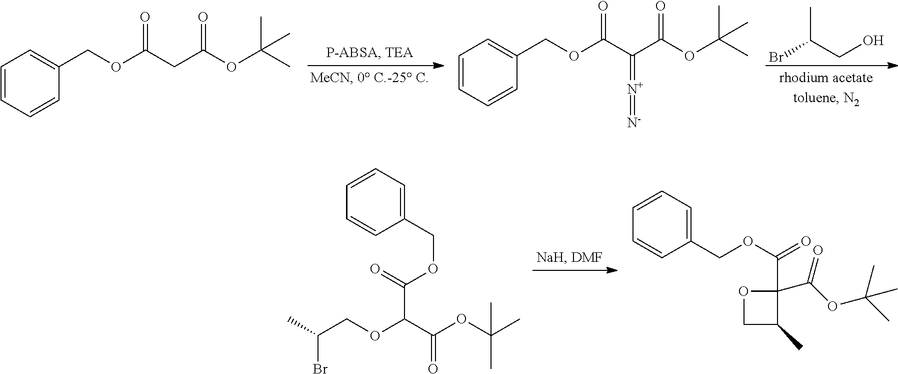

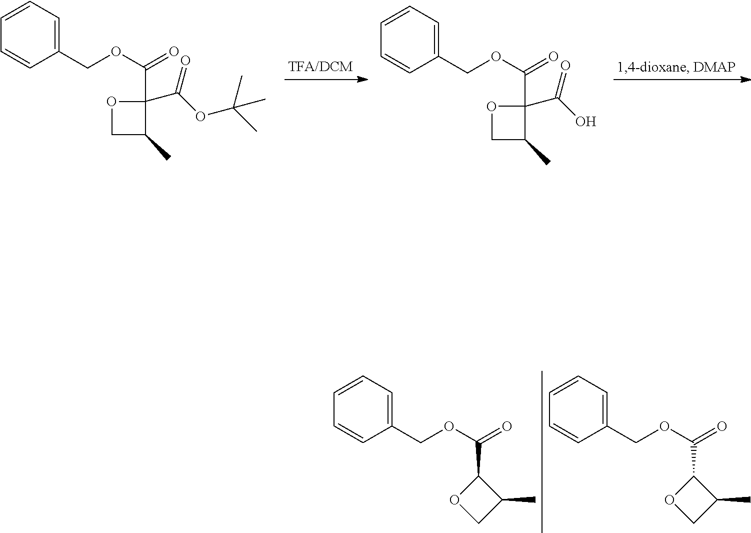

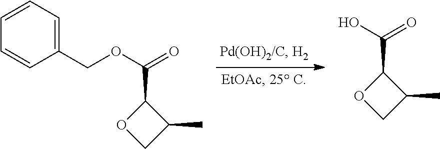

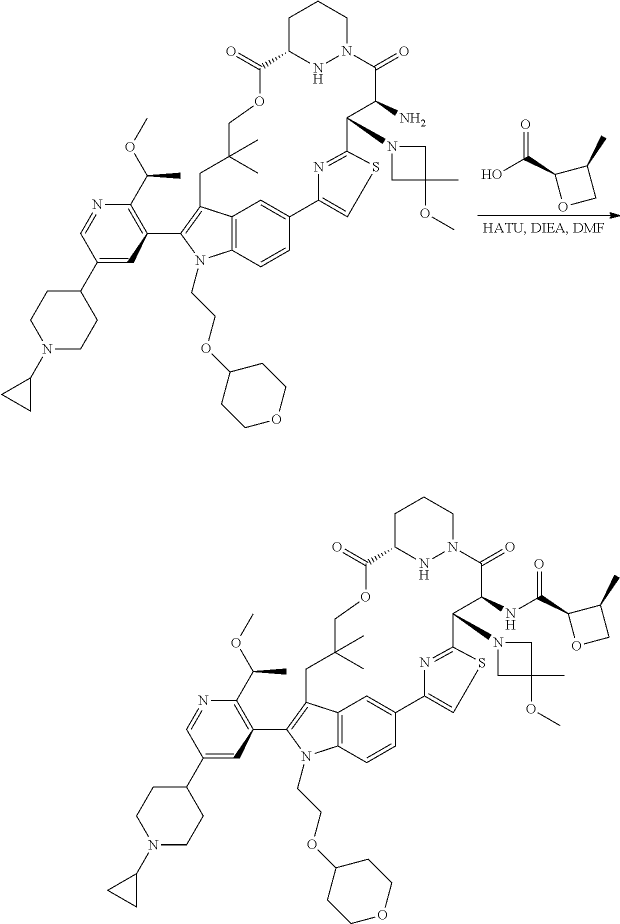

### Step 1

To a stirred mixture of benzyl tert-butyl malonate (450 g, 1797.886 mmol) and 4-acetamidobenzene-1-sulfonyl azide (431.92 g, 1797.886 mmol) in MeCN (11 L) was added EtsN (545.80 g, 5393.658 mmol) dropwise at 0° C. under air atmosphere. The resulting mixture was stirred for 16 h at 25° C. under air atmosphere. The resulting mixture was filtered, and the filter cake was washed with EtOAc (2×500 mL). The filtrate was concentrated under reduced pressure. The residue was purified by silica gel column chromatography, eluted with PE/EtOAc (10:1) to afford 1-benzyl 3-(tert-butyl) 2-diazomalonate (465 g, 93.61%) as a light yellow oil. LCMS (ESI): m/z [M+H] calc'd for C₁₄H₁₆N₂O₄ 276.11; found 276.11.

### Step 2

A solution of Rh₂(OAc)₄ (10.24 g, 23.164 mmol) in toluene (3200 mL) was treated with (2R)-2-bromopropan-1-ol (160.98 g, 1158.195 mmol) for 10 min at 0° C. under a nitrogen atmosphere followed by the addition of 1-benzyl 3-(tert-butyl) 2-diazomalonate (320 g, 1158.195 mmol) dropwise at 0° C. The resulting mixture was stirred for 4 h at 60° C. under a nitrogen atmosphere. The resulting mixture was filtered, the filter cake was washed with EtOAc (2×300 mL). The filtrate was concentrated under reduced pressure. The residue was purified by silica gel column chromatography, eluted with PE/EtOAc (10:1) to afford 1-benzyl 3-(tert-butyl) 2-((R)-2-bromopropoxy) malonate (300 g, 66.88%) as a light yellow oil. LCMS (ESI): m/z [M+H] calc'd for C₁₇H₂₃BrO₅ 386.07; found 386.07.

### Step 3

To a stirred mixture of 1-benzyl 3-(tert-butyl) 2-((R)-2-bromopropoxy) malonate (300 g, 774.653 mmol) in DMF (6 L) was added NaH (14.87 g, 619.722 mmol) in portions at 0° C. under air atmosphere. The resulting mixture was stirred for 5 h at 25° C. under air atmosphere. The reaction was quenched with sat. aq. KHSO₄ at 0° C. The resulting mixture was extracted with EtOAc (3×6 L). The combined organic layers were washed with H₂O (2×4 L), dried over anhydrous Na₂SO₄. After filtration, the filtrate was concentrated under reduced pressure. The residue was purified by silica gel column chromatography, eluted with PE/EtOAc (5:1) to afford 2-benzyl 2-(tert-butyl) (3S)-3-methyloxetane-2,2-dicarboxylate (144 g, 60.68%) as a light yellow oil. LCMS (ESI): m/z [M+H] calc'd for C₁₇H₂₂O₅ 306.15; found 306.15.

### Step 4

To a stirred mixture of 2-benzyl 2-(tert-butyl) (3S)-3-methyloxetane-2,2-dicarboxylate (30 g, 97.925 mmol) in CH₂Cl₂ (30 mL) was added TFA (10 mL) dropwise at 0° C. under an air atmosphere. The resulting mixture was stirred for 2 h at 25° C. under air atmosphere. The reaction was quenched with sat. aq. NaHCO₃ at 0° C. The aqueous layer was extracted with EtOAc (3×100 mL). The resulting mixture was concentrated under reduced pressure. This resulted in (3S)-2-((benzyloxy) carbonyl)-3-methyloxetane-2-carboxylic acid as a light yellow oil. The crude product was used in the next step directly without further purification. LCMS (ESI): m/z [M+H] calc'd for C₁₃H₁₄O₅ 250.08; found 250.08.

### Step 5

A mixture of (3S)-2-((benzyloxy) carbonyl)-3-methyloxetane-2-carboxylic acid (31.2 g, 124.675 mmol) and DMAP (30.46 g, 249.350 mmol) in 1,4-dioxane (300 mL) was stirred for 16 h at 100° C. under air atmosphere. The resulting mixture was filtered, the filter cake was washed with EtOAc (3×50 mL). The filtrate was concentrated under reduced pressure. The residue was purified by silica gel column chromatography, eluted with PE/EtOAc (9:1) to afford benzyl (2R,3S)-3-methyloxetane-2-carboxylate A (6 g, 23.33%) as a colorless oil and B (6.6 g, 25.67) as a colorless oil. LCMS (ESI): m/z [M+H] calc'd for C₁₂H₁₄O₃ 206.09; found 206.09.

### Step 6

A mixture of benzyl (2R,3S)-3-methyloxetane-2-carboxylate (1 g, 4.85 mmol) and Pd(OH)₂/C (250 mg) in EtOAc (10 mL) was stirred for 16 h at 100° C. under a hydrogen atmosphere. The resulting mixture was filtered, the filter cake was washed with EtOAc (3×10 mL). The filtrate was concentrated under reduced pressure, which afforded (2R,3S)-3-methyloxetane-2-carboxylic acid (500 mg, 89% yield) as colorless oil. LCMS (ESI): m/z [M+H] calc'd for C₅H₈O₃ 116.05; found 116.05.

### Step 7

Into a 100 mL round-bottom flask were added (2R,3S)-3-methyloxetane-2-carboxylic acid (19.97 mg, 0.164 mmol), DMF (10 mL), DIPEA (211.51 mg, 1.640 mmol), HATU (124.45 mg, 0.328 mmol) and (63S,3S,4S,Z)-4-amino-12-(5-(1-cyclopropylpiperidin-4-yl)-2-((S)-1-methoxyethyl)pyridin-3-yl)-3-(3-methoxy-3-methylazetidin-1-yl)-10,10-dimethyl-11-(2-((tetrahydro-2H-pyran-4-yl)oxy)ethyl)-61,62,63,64,65,66-hexahydro-11H-8-oxa-2(4,2)-thiazola-1(5,3)-indola-6(1,3)-pyridazinacycloundecaphane-5,7-dione (156 mg, 0.164 mmol) at 25° C. The reaction was monitored by LCMS. The aqueous layer was extracted with EtOAc (3×10 mL). The resulting mixture was concentrated under reduced pressure. The crude product (150 mg) was purified by Prep-HPLC to afford (2R,3S)—N-((63S,3S,4S,Z)-12-(5-(1-cyclopropylpiperidin-4-yl)-2-((S)-1-methoxyethyl)pyridin-3-yl)-3-(3-methoxy-3-methylazetidin-1-yl)-10,10-dimethyl-5,7-dioxo-11-(2-((tetrahydro-2H-pyran-4-yl)oxy)ethyl)-61,62,63,64,65,66-hexahydro-11H-8-oxa-2(4,2)-thiazola-1(5,3)-indola-6(1,3)-pyridazinacycloundecaphane-4-yl)-3-methyloxetane-2-carboxamide (32.4 mg, 17.63%) as a white solid. LCMS (ESI): m/z [M+H] calc'd for C₅₇H₇₈N₈O₉S 1050.56; found 1050.56.

### Example 10: Synthesis of (2R,3S)—N-((6³S,3S,4S,Z)-12-(5-((S)-1-cyclopropylpyrrolidin-3-yl)-2-(S)-1-methoxyethyl)pyridin-3-yl)-3-(3-methoxy-3-methylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyloxetane-2-carboxamide

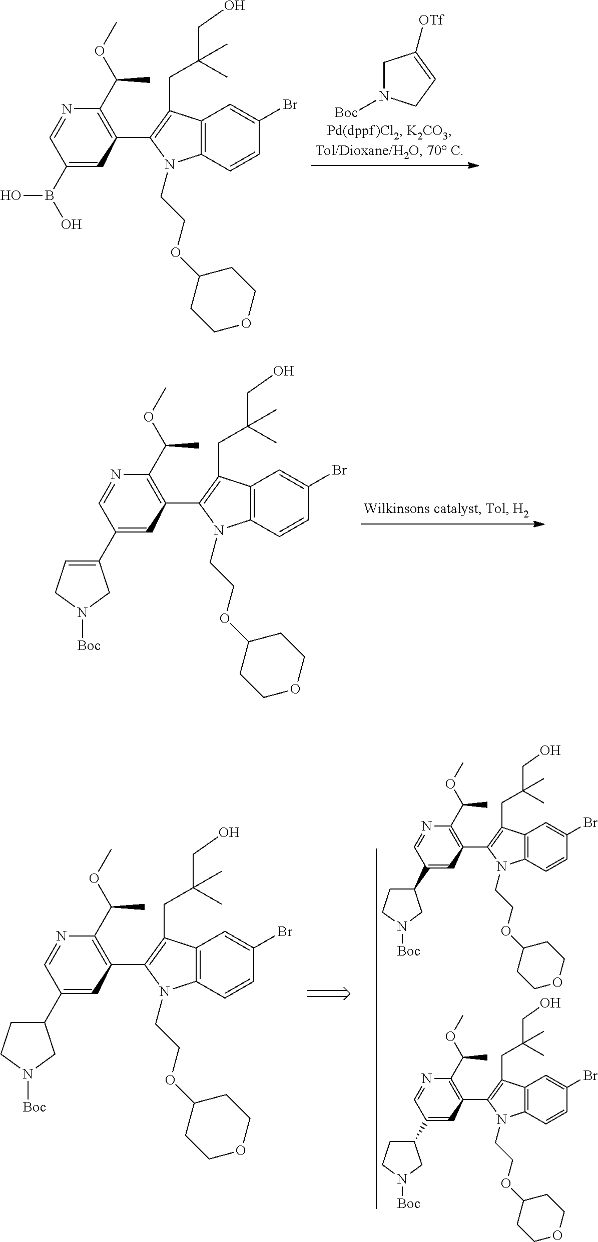

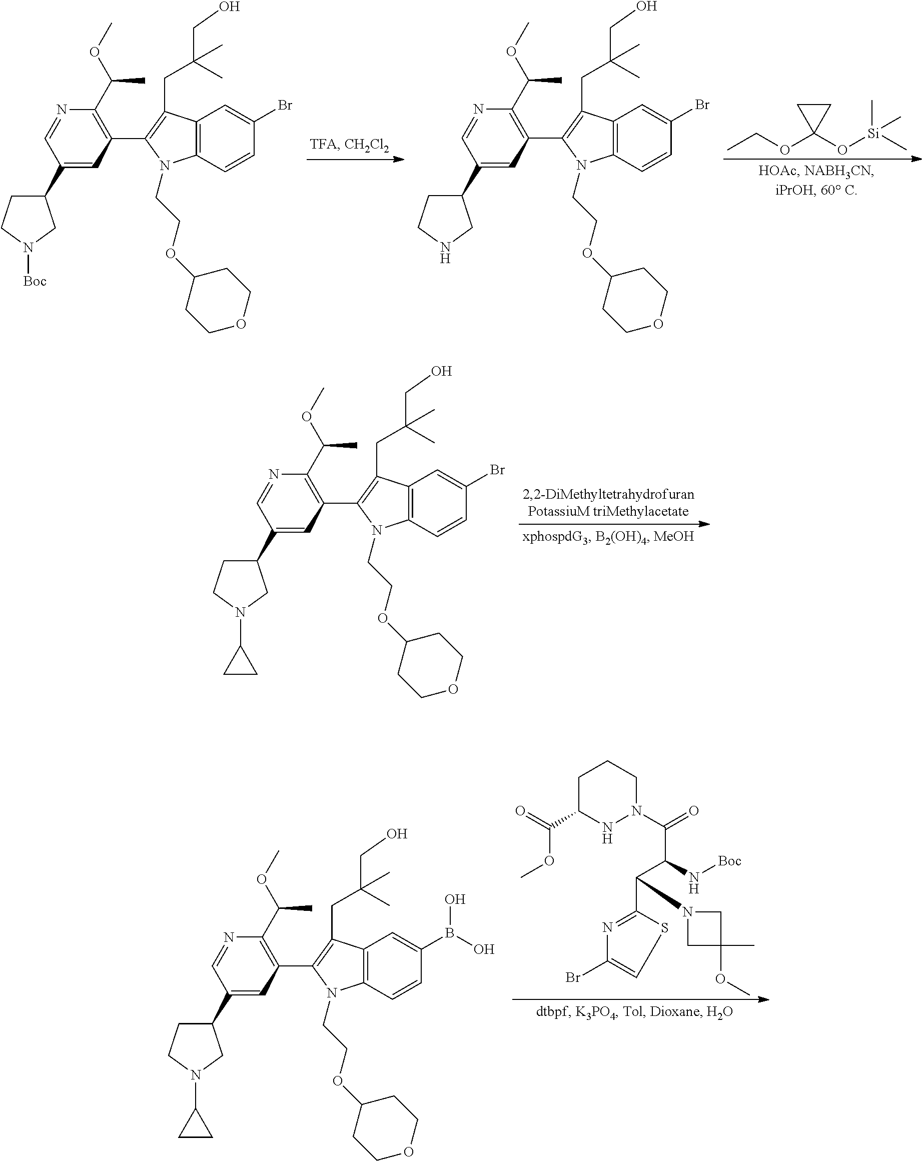

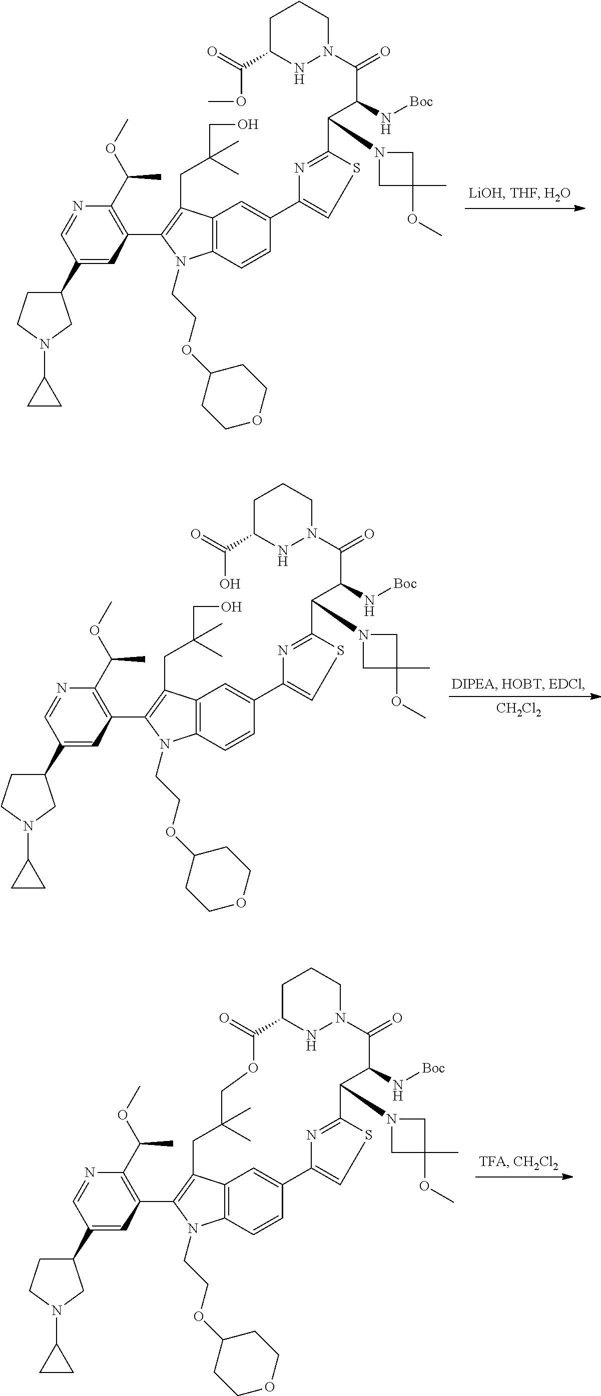

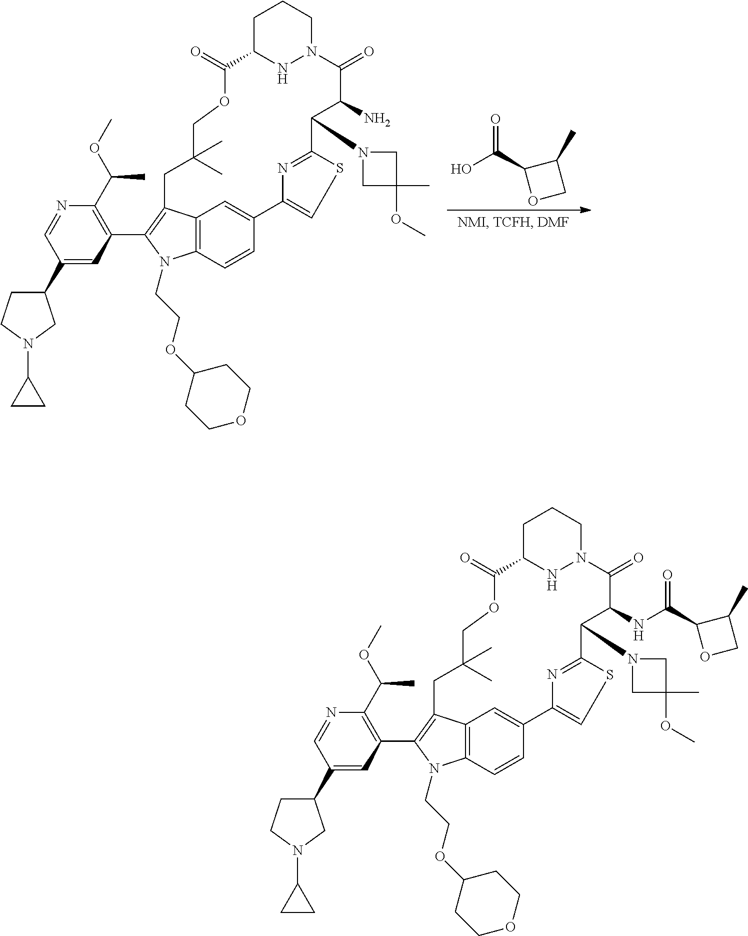

### Step 1

A solution of 5-[5-bromo-3-(3-hydroxy-2,2-dimethylpropyl)-1-[2-(oxan-4-yloxy)ethyl]indol-2-yl]-6-[(1S)-1-methoxyethyl]pyridin-3-ylboronic acid (12 g, 23.20 mmol), tert-butyl 3-(trifluoromethanesulfonyloxy)-2,5-dihydropyrrole-1-carboxylate (36.81 g, 116.01 mmol), Pd(dppf)Cl₂ (1.70 g, 2.32 mmol) and K₂CO₃ (8.02 g, 58.01 mmol) in toluene (120 mL), dioxane (40 mL), H₂O (40 mL) was stirred for 2 h at 70° C. under nitrogen atmosphere. The resulting mixture was concentrated under reduced pressure. The residue was purified by silica gel column chromatography, eluted with PE/EtOAc (1:1) to afford tert-butyl 3-{5-[5-bromo-3-(3-hydroxy-2,2-dimethylpropyl)-1-[2-(oxan-4-yloxy)ethyl]indol-2-yl]-6-[(1S)-1-methoxyethyl]pyridin-3-yl}-2,5-dihydropyrrole-1-carboxylate (7 g, 42.39%) as a brown solid. LCMS (ESI): m/z [M+H] calc'd for C₃₇H₅₀BrN₃O₆ 712.29, found 712.40.

### Step 2

A solution of tert-butyl 3-{5-[5-bromo-3-(3-hydroxy-2,2-dimethylpropyl)-1-[2-(oxan-4-yloxy)ethyl]indol-2-yl]-6-[(1S)-1-methoxyethyl]pyridin-3-yl}-2,5-dihydropyrrole-1-carboxylate (3 g, 4.68 mmol), Tris(triphenylphosphine)phosphonium chloride (3 g, 3.24 mmol) in toluene (50 mL) was stirred for overnight at 70° C. under a hydrogen atmosphere. The resulting mixture was concentrated under reduced pressure. The residue was purified by silica gel column chromatography, eluted with PE/EtOAc (1:1) to afford tert-butyl 3-{5-[5-bromo-3-(3-hydroxy-2,2-dimethylpropyl)-1-[2-(oxan-4-yloxy)ethyl]indol-2-yl]-6-[(1S)-1-methoxyethyl]pyridin-3-yl}pyrrolidine-1-carboxylate (1.7 g, 53.66%) as a brown solid. The residue was purified by SFC to afford tert-butyl (3S)-3-{5-[5-bromo-3-(3-hydroxy-2,2-dimethylpropyl)-1-[2-(oxan-4-yloxy)ethyl]indol-2-yl]-6-[(1S)-1-methoxyethyl]pyridin-3-yl}pyrrolidine-1-carboxylate (700 mg, 41.17%) and tert-butyl (3R)-3-{5-[5-bromo-3-(3-hydroxy-2,2-dimethylpropyl)-1-[2-(oxan-4-yloxy)ethyl]indol-2-yl]-6-[(1S)-1-methoxyethyl]pyridin-3-yl}pyrrolidine-1-carboxylate (960 mg, 56.47%). LCMS (ESI): m/z [M+H] calc'd for C₃₇H₅₂BrN₃O₆ 714.30, found 714.50.

### Step 3

A mixture of tert-butyl (3S)-3-{5-[5-bromo-3-(3-hydroxy-2,2-dimethylpropyl)-1-[2-(oxan-4-yloxy)ethyl]indol-2-yl]-6-[(1S)-1-methoxyethyl]pyridin-3-yl}pyrrolidine-1-carboxylate (3.2 g, 4.48 mmol) in CH₂Cl₂ (22 mL) was stirred for 2 min at 0° C. under an air atmosphere followed by the addition of TFA (11 mL) dropwise at 0° C. The resulting mixture was stirred for 1 h at 25° C. The mixture was basified to pH 8 with saturated NaHCO₃(aq.). The resulting mixture was extracted with CH₂Cl₂ (1×50 mL), dried over anhydrous Na₂SO₄. After filtration, the filtrate was concentrated under reduced pressure. This resulted in 3-(5-bromo-2-(2-((S)-1-methoxyethyl)-5-((S)-pyrrolidin-3-yl) pyridin-3-yl)-1-(2-((tetrahydro-2H-pyran-4-yl)oxy)ethyl)-1H-indol-3-yl)-2,2-dimethylpropan-1-ol (3.4 g, crude) as a brown solid. The crude product was used in the next step directly without further purification. LCMS (ESI): m/z [M+H] calc'd for C₃₂H₄₄BrN₃O₄ 614.25, found 614.20.

### Step 4

A mixture of 3-(5-bromo-2-(2-((S)-1-methoxyethyl)-5-((S)-pyrrolidin-3-yl) pyridin-3-yl)-1-(2-((tetrahydro-2H-pyran-4-yl)oxy)ethyl)-1H-indol-3-yl)-2,2-dimethylpropan-1-ol (3.4 g, 5.53 mmol) and (1-ethoxycyclopropoxy)trimethylsilane (9.64 g, 55.32 mmol) and HOAc (3.32 g, 55.32 mmol) in iPrOH (35 mL) was stirred for 10 min at 0° C. under an air atmosphere followed by the addition of NABH₃CN (3.48 g, 55.32 mmol) dropwise at 0° C. The resulting mixture was stirred for 2 h at 60° C. The reaction was quenched by the addition of NaHCO₃ (50 mL) at 0° C. The resulting mixture was extracted with CH₂Cl₂ (3×50 mL), dried over anhydrous Na₂SO₄. After filtration, the filtrate was concentrated under reduced pressure. The residue was purified by silica gel column chromatography, eluted with PE/EtOAc (1:1) to afford 3-(5-bromo-2-(5-((S)-1-cyclopropylpyrrolidin-3-yl)-2-((S)-1-methoxyethyl)pyridin-3-yl)-1-(2-((tetrahydro-2H-pyran-4-yl)oxy)ethyl)-1H-indol-3-yl)-2,2-dimethylpropan-1-ol (1.8 g, 49.70%) as a brown solid. LCMS (ESI): m/z [M+H] calc'd for C₃₅H₄₈BrN₃O₄ 654.28, found 654.25.

### Step 5

A mixture of 3-(5-bromo-2-(5-((S)-1-cyclopropylpyrrolidin-3-yl)-2-((S)-1-methoxyethyl)pyridin-3-yl)-1-(2-((tetrahydro-2H-pyran-4-yl)oxy)ethyl)-1H-indol-3-yl)-2,2-dimethylpropan-1-ol (1.8 g, 2.75 mmol) and XPhos Pd G3 (116.36 mg, 0.14 mmol) and B₂(OH)₄ (492.97 mg, 5.50 mmol) and potassium trimethylsilanolate (1058.13 mg, 8.25 mmol) in MeOH (7 mL) and 2-Methyltetrahydrofuran (14 mL) was stirred for 2 h at 40° C. under a nitrogen atmosphere. The resulting mixture was filtered, the filter cake was washed with EtOAc (1×300 mL). The resulting mixture was washed with H₂O (1×300 mL), dried over anhydrous Na₂SO₄. After filtration, the filtrate was concentrated under reduced pressure. This resulted in (2-(5-((S)-1-cyclopropylpyrrolidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) boronic acid (1.7 g, crude) as a black solid. The crude product was used in the next step directly without further purification. LCMS (ESI): m/z [M+H] calc'd for C₃₅H₅₀BN₃O₆ 620.38, found 620.40.

### Step 6

A mixture of (2-(5-((S)-1-cyclopropylpyrrolidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) boronic acid (740 mg, 1.25 mmol) and methyl(S)-1-((2S,3S)-3-(4-bromothiazol-2-yl)-2-((tert-butoxycarbonyl)amino)-3-(3-methoxy-3-methylazetidin-1-yl) propanoyl) hexahydropyridazine-3-carboxylate (792.98 mg, 1.38 mmol) and Pd(dtbpf)Cl₂ (162.99 mg, 0.25 mmol) and K₃PO₄ (796.27 mg, 3.75 mmol) in toluene (6 mL) and 1,4-dioxane (2 mL) and H₂O (2 mL) was stirred for 2 h at 80° C. under a nitrogen atmosphere. The resulting mixture was diluted with CH₂Cl₂ (50 mL). The resulting mixture was washed with H₂O (1×50 mL), dried over anhydrous Na₂SO₄. After filtration, the filtrate was concentrated under reduced pressure. The residue was purified by silica gel column chromatography, eluted with PE/EtOAc (3% NEt₃) (1:1) to afford methyl(S)-1-((2S,3S)-2-((tert-butoxycarbonyl)amino)-3-(4-(2-(5-((S)-1-cyclopropylpyrrolidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(3-methoxy-3-methylazetidin-1-yl) propanoyl) hexahydropyridazine-3-carboxylate (700 mg, 52.25%) as a brown solid. LCMS (ESI): m/z [M+H] calc'd for C₅₇H₈₂N₈O₁₀S 1071.59, found 1072.15.

### Step 7

A mixture of methyl(S)-1-((2S,3S)-2-((tert-butoxycarbonyl)amino)-3-(4-(2-(5-((S)-1-cyclopropylpyrrolidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(3-methoxy-3-methylazetidin-1-yl) propanoyl) hexahydropyridazine-3-carboxylate (700 mg, 0.65 mmol) in THF (3.5 mL, 43.20 mmol) was stirred for 2 min at 0° C. under an air atmosphere followed by the addition of LiOH. H₂O (109.66 mg, 2.61 mmol) and H₂O (3.5 mL, 194.28 mmol) in portions at 0° C. The resulting mixture was stirred for 1 h at 25° C. The mixture was acidified to pH 5 with citric acid. The resulting mixture was extracted with CH₂Cl₂ (3×50 mL), dried over anhydrous Na₂SO₄. After filtration, the filtrate was concentrated under reduced pressure. This resulted in(S)-1-((2S,3S)-2-((tert-butoxycarbonyl)amino)-3-(4-(2-(5-((S)-1-cyclopropylpyrrolidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(3-methoxy-3-methylazetidin-1-yl) propanoyl) hexahydropyridazine-3-carboxylic acid (710 mg, crude) as a brown solid. The crude product was used in the next step directly without further purification. LCMS (ESI): m/z [M+H] calc'd for C₅₆H₈₀N₈O₁₀S 1057.57, found 1058.60.

### Step 8

A mixture of(S)-1-((2S,3S)-2-((tert-butoxycarbonyl)amino)-3-(4-(2-(5-((S)-1-cyclopropylpyrrolidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(3-methoxy-3-methylazetidin-1-yl) propanoyl) hexahydropyridazine-3-carboxylic acid (700 mg, 0.66 mmol) in CH₂Cl₂ (70 mL) was stirred for 2 min at 0° C. under an air atmosphere followed by the addition of DIPEA (3.42 g, 26.48 mmol) and HOBT (0.89 g, 6.62 mmol) and EDCl (3.81 g, 19.86 mmol) in portions at 0° C. The resulting mixture was stirred for 16 h at 25° C. The resulting mixture was washed with citric acid (1×50 mL). The resulting mixture was washed with of NaHCO₃ (1×50 mL). The resulting mixture was washed with H₂O (1×50 mL), dried over anhydrous Na₂SO₄. After filtration, the filtrate was concentrated under reduced pressure. The residue was purified by silica gel column chromatography, eluted with PE/EtOAc (3% NEt₃) (1:1) to afford tert-butyl ((6³S,3S,4S,Z)-1²-(5-((S)-1-cyclopropylpyrrolidin-3-yl)-2-((S)-1-methoxyethyl)pyridin-3-yl)-3-(3-methoxy-3-methylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (300 mg, 43.60%) as a brown solid. LCMS (ESI): m/z [M+H] calc'd for C₅₆H₇₈N₈O₉S 1039.56, found 1040.05.

### Step 9

A mixture of tert-butyl ((6³S,3S,4S,Z)-1²-(5-((S)-1-cyclopropylpyrrolidin-3-yl)-2-((S)-1-methoxyethyl)pyridin-3-yl)-3-(3-methoxy-3-methylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (300 mg, 0.29 mmol) in CH₂Cl₂ (2 mL) was stirred for 2 min at 0° C. under an air atmosphere followed by the addition of TFA (1 mL) dropwise at 0° C. The resulting mixture was stirred for 1 h at 25° C. The mixture was basified to pH 8 with saturated NaHCO₃(aq.). The resulting mixture was extracted with CH₂Cl₂ (3×50 mL), dried over anhydrous Na₂SO₄. After filtration, the filtrate was concentrated under reduced pressure. This resulted in (6³S,3S,4S,Z)-4-amino-1²-(5-((R)-1-cyclopropylpyrrolidin-3-yl)-2-((S)-1-methoxyethyl)pyridin-3-yl)-3-(3-methoxy-3-methylazetidin-1-yl)-10,10-dimethyl-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (300 mg, crude) as a brown solid. The crude product was used in the next step directly without further purification. LCMS (ESI): m/z [M+H] calc'd for C₅₁H₇₀N₈O₇S 939.51, found 939.50.

### Step 10

A solution of (2R,3S)-3-methyloxetane-2-carboxylic acid (12.36 mg, 0.11 mmol) and DIPEA (137.61 mg, 1.06 mmol) in DMF (2 mL) was stirred for 5 min at 0° C. under an air atmosphere. To the above mixture was added (6³S,3S,4S,Z)-4-amino-1²-(5-((R)-1-cyclopropylpyrrolidin-3-yl)-2-((S)-1-methoxyethyl)pyridin-3-yl)-3-(3-methoxy-3-methylazetidin-1-yl)-10,10-dimethyl-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (100 mg, 0.11 mmol) and HATU (80.97 mg, 0.21 mmol) in portions over 1 min at 0° C. The resulting mixture was stirred for additional 1.5 h at room temperature. The resulting mixture was washed with brine (3×5 mL), dried over anhydrous Na₂SO₄. After filtration, the filtrate was concentrated under reduced pressure. The crude product (12 mg) was purified by Prep-HPLC to afford (2R,3S)—N-((6³S,3S,4S,Z)-12-(5-((S)-1-cyclopropylpyrrolidin-3-yl)-2-((S)-1-methoxyethyl)pyridin-3-yl)-3-(3-methoxy-3-methylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyloxetane-2-carboxamide (12 mg, 10.81%) as a white solid. LCMS (ESI): m/z [M+H] calc'd for C₅₆H₇₆N₈O₉S 1037.55, found 1037.60. ¹H NMR (400 MHZ, DMSO-d6) δ 8.64 (d, J=2.2 Hz, 1H), 8.51-8.45 (m, 1H), 8.07 (d, J=8.9 Hz, 1H), 7.95 (s, 1H), 7.75 (dd, J=8.6, 1.6 Hz, 1H), 7.66-7.57 (m, 2H), 5.56 (d, J=8.8 Hz, 1H), 5.19 (d, J=12.1 Hz, 1H), 4.89-4.82 (m, 1H), 4.59 (d, J=5.9 Hz, 1H), 4.47 (d, J=14.8 Hz, 1H), 4.40-4.19 (m, 6H), 3.64 (d, J=10.9 Hz, 2H), 3.55 (s, 4H), 3.25 (d, J=11.5 Hz, 4H), 3.15-3.01 (m, 10H), 2.99-2.92 (m, 2H), 2.90-2.74 (m, 3H), 2.69 (dd, J=9.1, 6.5 Hz, 1H), 2.44 (d, J=14.2 Hz, 1H), 2.37-2.26 (m, 1H), 2.13 (d, J=12.1 Hz, 1H), 1.82 (d, J=19.0 Hz, 2H), 1.77-1.67 (m, 2H), 1.51 (d, J=17.1 Hz, 3H), 1.41 (s, 3H), 1.34 (dd, J=6.6, 1.8 Hz, 6H), 1.27-1.18 (m, 1H), 1.12-1.00 (m, 2H), 0.93 (s, 3H), 0.41 (dd, J=6.7, 4.2 Hz, 2H), 0.35-0.22 (m, 5H).

### Example 10: Synthesis of a Compound of Table 1

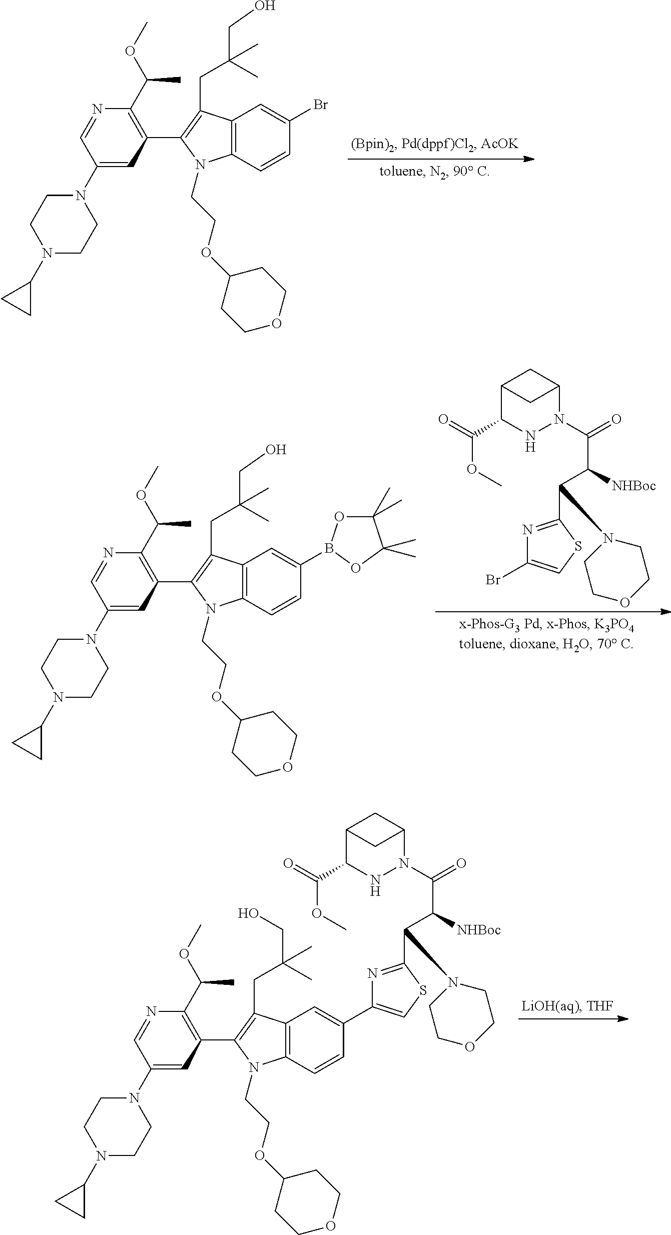

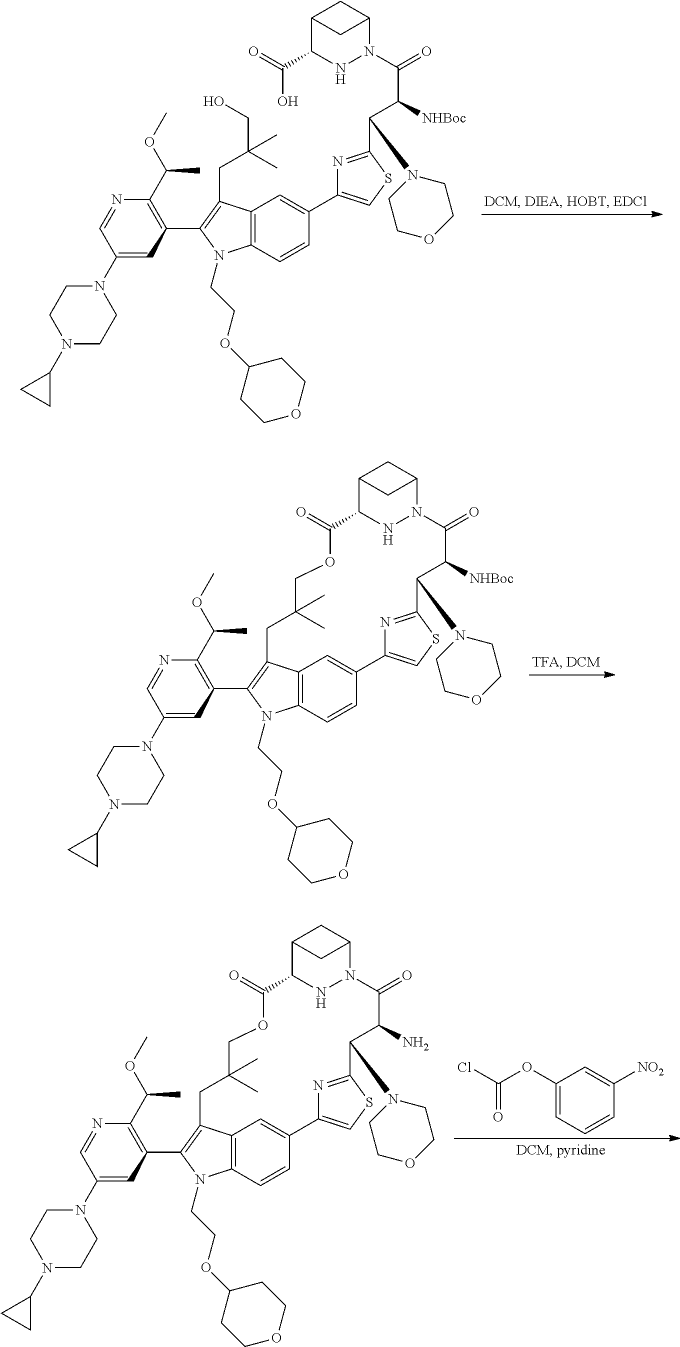

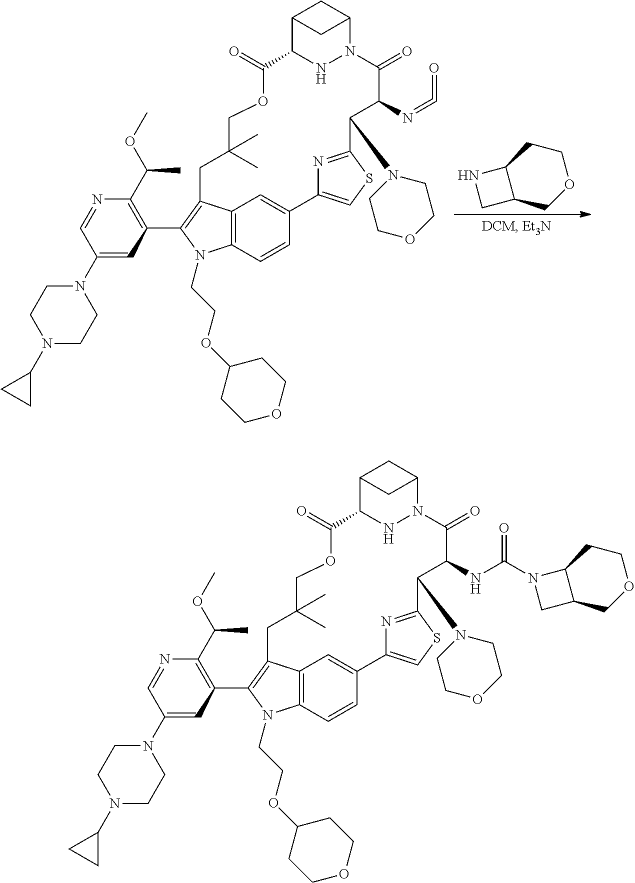

### Step 1

Into a 50 mL sealed tube were added 3-[(2M)-5-bromo-2-[5-(4-cyclopropylpiperazin-1-yl)-2-[(1S)-1-methoxyethyl]pyridin-3-yl]-1-[2-(oxan-4-yloxy)ethyl]indol-3-yl]-2,2-dimethylpropan-1-ol (500 mg, 0.747 mmol), methyl 4-(4,4,5,5-tetramethyl-1,3,2-dioxaborolan-2-yl)pyridine-2-carboxylate (392.86 mg, 1.494 mmol), AcOK (219.82 mg, 2.241 mmol), Pd(dppf)Cl₂ (163.89 mg, 0.224 mmol) and toluene (5 mL) at 20° C. The resulting mixture was stirred for 1 h at 90° C. under an argon atmosphere. The reaction was quenched by the addition of H₂O (40 mL) at room temperature. The resulting mixture was extracted with EtOAc (3×50 mL). The combined organic layers dried over anhydrous Na₂SO₄. After filtration, the filtrate was concentrated under reduced pressure. The residue was purified by Prep-TLC (CH₂Cl₂/MeOH 12:1) to afford 3-[(2M)-2-[5-(4-cyclopropylpiperazin-1-yl)-2-[(1S)-1-methoxyethyl]pyridin-3-yl]-1-[2-(oxan-4-yloxy)ethyl]-5-(4,4,5,5-tetramethyl-1,3,2-dioxaborolan-2-yl) indol-3-yl]-2,2-dimethylpropan-1-ol (400 mg, 63.53%) as a light yellow solid. LCMS (ESI): m/z [M+H] calc'd for C₄₁H₆₁BN₄O₆ 716.5; found 716.5.

### Step 2

To a solution of 3-[(2M)-2-[5-(4-cyclopropylpiperazin-1-yl)-2-[(1S)-1-methoxyethyl]pyridin-3-yl]-1-[2-(oxan-4-yloxy)ethyl]-5-(4,4,5,5-tetramethyl-1,3,2-dioxaborolan-2-yl) indol-3-yl]-2,2-dimethylpropan-1-ol (360 mg, 0.502 mmol) and ethyl (4S)-2-[(2S,3S)-3-(4-bromo-1,3-thiazol-2-yl)-2-[(tert-butoxycarbonyl)amino]-3-[(3S)-3-fluoropyrrolidin-1-yl]propanoyl]-2,3-diazabicyclo[3.1.1]heptane-4-carboxylate (326.24 mg, 0.552 mmol) in toluene (0.9 mL), 1,4-dioxane (0.3 mL), H₂O (0.3 mL) were added K₃PO₄ (319.83 mg, 1.506 mmol) and dicyclohexyl [2′,4′,6′-tris(propan-2-yl)-[1,1′-biphenyl]-2-yl]phosphane (47.89 mg, 0.100 mmol). 3rd Generation XPhos precatalyst (42.51 mg, 0.001 mmol) at 20° C. After stirring for 2 h at 70° C. under an argon atmosphere, the resulting mixture was concentrated under reduced pressure. The residue was purified by silica gel column chromatography, eluted with CH₂Cl₂/MeOH (10:1) to afford methyl (4S)-2-[(2S,3S)-2-[(tert-butoxycarbonyl)amino]-3-{4-[(2M)-2-[5-(4-cyclopropylpiperazin-1-yl)-2-[(1S)-1-methoxyethyl]pyridin-3-yl]-3-(3-hydroxy-2,2-dimethylpropyl)-1-[2-(oxan-4-yloxy)ethyl]indol-5-yl]-1,3-thiazol-2-yl}-3-[(3S)-3-fluoropyrrolidin-1-yl]propanoyl]-2,3-diazabicyclo[3.1.1]heptane-4-carboxylate (230 mg, 33.72%) as a light brown solid. LCMS (ESI): m/z [M+H] calc'd for C₅₇H₈₁N₉O₁₀S 1083.58; found 1083.58.

### Step 3

Into a 100 mL round-bottom flask were added ethyl (4S)-2-[(2S,3S)-2-[(tert-butoxycarbonyl)amino]-3-{4-[(2M)-2-[5-(4-cyclopropylpiperazin-1-yl)-2-[(1S)-1-methoxyethyl]pyridin-3-yl]-3-(3-hydroxy-2,2-dimethylpropyl)-1-[2-(oxan-4-yloxy)ethyl]indol-5-yl]-1,3-thiazol-2-yl}-3-(morpholin-4-yl) propanoyl]-2,3-diazabicyclo[3.1.1]heptane-4-carboxylate (140 mg, 0.127 mmol), THF (0.7 mL) and LiOH (1 N, aq.) (0.7 mL, 0.700 mmol) at 0° C. The resulting mixture was stirred for 1 h at 20° C. under air atmosphere. The reaction was quenched with H₂O at 20° C. The residue was acidified to pH 6 with HCl (1 mol/L). The aqueous layer was extracted with EtOAc (3×20 mL). The combined organic layers dried over anhydrous Na₂SO₄. After filtration, the filtrate was concentrated under reduced pressure. This resulted in (4S)-2-[(2S,3S)-2-[(tert-butoxycarbonyl)amino]-3-{4-[(2M)-2-[5-(4-cyclopropylpiperazin-1-yl)-2-[(1S)-1-methoxyethyl]pyridin-3-yl]-3-(3-hydroxy-2,2-dimethylpropyl)-1-[2-(oxan-4-yloxy)ethyl]indol-5-yl]-1,3-thiazol-2-yl}-3-(morpholin-4-yl) propanoyl]-2,3-diazabicyclo[3.1.1]heptane-4-carboxylic acid (110 mg, 48.38%) as a light yellow solid. LCMS (ESI): m/z [M+H] calc'd for C₅₆H₇₉N₉O₁₀S 1069.57; found 1069.57.

### Step 4

Into a 100 mL round-bottom flask were added (4S)-2-[(2S,3S)-2-[(tert-butoxycarbonyl)amino]-3-{4-[(2M)-2-[5-(4-cyclopropylpiperazin-1-yl)-2-[(1S)-1-methoxyethyl]pyridin-3-yl]-3-(3-hydroxy-2,2-dimethylpropyl)-1-[2-(oxan-4-yloxy)ethyl]indol-5-yl]-1,3-thiazol-2-yl}-3-(morpholin-4-yl) propanoyl]-2,3-diazabicyclo[3.1.1]heptane-4-carboxylic acid (190 mg, 0.178 mmol), CH₂Cl₂ (38 mL), DIPEA (688.28 mg, 5.340 mmol), HOBT (119.93 mg, 0.890 mmol), and EDCl (1020.86 mg, 5.340 mmol) at 20° C. The resulting mixture was stirred for 12 h at 30° C. under air atmosphere. The reaction was quenched with H₂O at room temperature. The resulting mixture was extracted with CH₂Cl₂ (3×50 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford tert-butyl ((6⁴S,3S,4S,Z)-12-(5-(4-cyclopropylpiperazin-1-yl)-2-((S)-1-methoxyethyl)pyridin-3-yl)-10,10-dimethyl-3-morpholino-5,7-dioxo-11-(2-((tetrahydro-2H-pyran-4-yl)oxy)ethyl)-11H-8-oxa-6²,6³-diaza-2(4,2)-thiazola-1(5,3)-indola-6 (2,4)-bicyclo[3.1.1]heptanacycloundecaphane-4-yl) carbamate (90 mg, 40.95%) as a light yellow solid. LCMS (ESI): m/z [M+H] calc'd for C₅₆H₇₇N₉O₉S 1051.56; found 1051.56.

### Step 5

Into a 100 mL round-bottom flask were added tert-butyl ((6⁴S,3S,4S,Z)-12-(5-(4-cyclopropylpiperazin-1-yl)-2-((S)-1-methoxyethyl)pyridin-3-yl)-10,10-dimethyl-3-morpholino-5,7-dioxo-11-(2-((tetrahydro-2H-pyran-4-yl)oxy)ethyl)-11H-8-oxa-6²,6³-diaza-2(4,2)-thiazola-1(5,3)-indola-6 (2,4)-bicyclo[3.1.1]heptanacycloundecaphane-4-yl) carbamate (200 mg, 0.190 mmol), CH₂Cl₂ (6 mL), and TFA (2 mL, 26.926 mmol) at 0° C. The resulting mixture was stirred for 1 h at 0° C. under air atmosphere. The reaction was quenched by the addition of aq. NaHCO₃ (5 mL) at 0° C. The resulting mixture was extracted with CH₂Cl₂ (3×20 mL). The combined organic layers were washed with brine (1×1 30 mL), dried over anhydrous Na₂SO₄. After filtration, the filtrate was concentrated under reduced pressure. This resulted in (6⁴S,3S,4S,Z)-4-amino-1²-(5-(4-cyclopropylpiperazin-1-yl)-2-((S)-1-methoxyethyl)pyridin-3-yl)-10,10-dimethyl-3-morpholino-11-(2-((tetrahydro-2H-pyran-4-yl)oxy)ethyl)-11H-8-oxa-6²,6³-diaza-2(4,2)-thiazola-1(5,3)-indola-6 (2,4)-bicyclo[3.1.1]heptanacycloundecaphane-5,7-dione (160 mg, 88.41%) as a yellow solid. LCMS (ESI): m/z [M+H] calc'd for C₅₁H₆₉N₉O₇S 951.50; found 951.50.

### Step 6

Into a 100 mL round-bottom flask were added (64S,3S,4S,Z)-4-amino-12-(5-(4-cyclopropylpiperazin-1-yl)-2-((S)-1-methoxyethyl)pyridin-3-yl)-10,10-dimethyl-3-morpholino-1¹-(2-((tetrahydro-2H-pyran-4-yl)oxy)ethyl)-11H-8-oxa-6²,6³-diaza-2(4,2)-thiazola-1(5,3)-indola-6 (2,4)-bicyclo[3.1.1]heptanacycloundecaphane-5,7-dione (160 mg, 0.168 mmol, 1 equiv), CH₂Cl₂ (3 mL), Pyridine (66.45 mg, 0.840 mmol, 5 equiv) and 3-nitrophenyl carbonochloridate (67.73 mg, 0.336 mmol, 2 equiv) at 0° C. The resulting mixture was stirred for 1 h at 0° C. under air atmosphere. The resulting mixture was concentrated under vacuum. This resulted in the product (160 mg, 97.34%) as a yellow solid. LCMS (ESI): m/z [M+H] calc'd for C₅₂H₆₇N₉O₈S 977.48; found 977.48.

### Step 7

Into a 100 mL round-bottom flask were added (6⁴S,3S,4S,Z)-12-(5-(4-cyclopropylpiperazin-1-yl)-2-((S)-1-methoxyethyl)pyridin-3-yl)-4-isocyanato-10,10-dimethyl-3-morpholino-1¹-(2-((tetrahydro-2H-pyran-4-yl)oxy)ethyl)-1¹H-8-oxa-6²,6³-diaza-2(4,2)-thiazola-1(5,3)-indola-6 (2,4)-bicyclo[3.1.1]heptanacycloundecaphane-5,7-dione (160 mg, 0.164 mmol, 1 equiv), CH₂Cl₂ (4 mL), NEt₃ (165.51 mg, 1.640 mmol, 10 equiv) and (1S,6S)-3-oxa-7-azabicyclo[4.2.0]octane (37.02 mg, 0.328 mmol, 2 equiv) at 0° C. The resulting mixture was stirred for 1 h at 0° C. under air atmosphere. The reaction was quenched with Water at room temperature. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. The crude product (140 mg) was purified by Prep-HPLC to afford the product (10.6 mg, 5.94%, 97.5% purity at 220 nm, 98.1% purity at 254 nm) as a white solid. LCMS (ESI): m/z [M+H] calc'd for C₅₈H₇₈N₁₀O₉S 1090.57; found 1090.57.

### Synthesis of (2R)—N-((6³S,3S,4S,Z)-12-(5-(4-cyclopropylpiperazin-1-yl)-2-((S)-1-methoxyethyl)pyridin-3-yl)-3-(2,2-difluoroethoxy)-10,10-dimethyl-5,7-dioxo-1¹-(2-((tetrahydro-2H pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3,3-difluoro-2-((R)-1-hydroxyethyl) azetidine-1-carboxamide

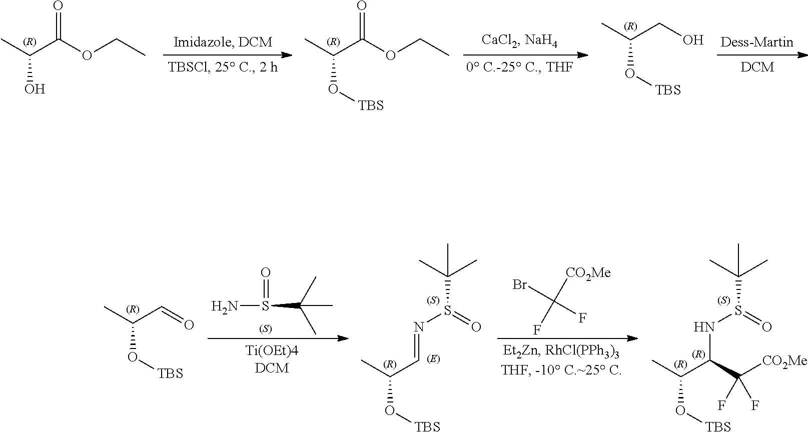

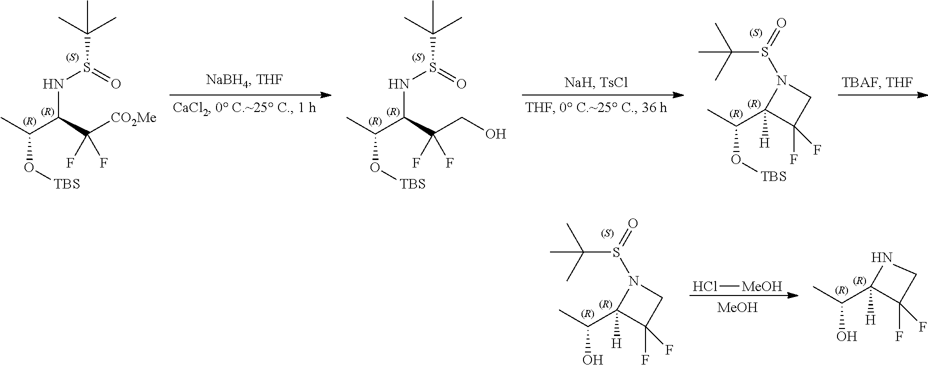

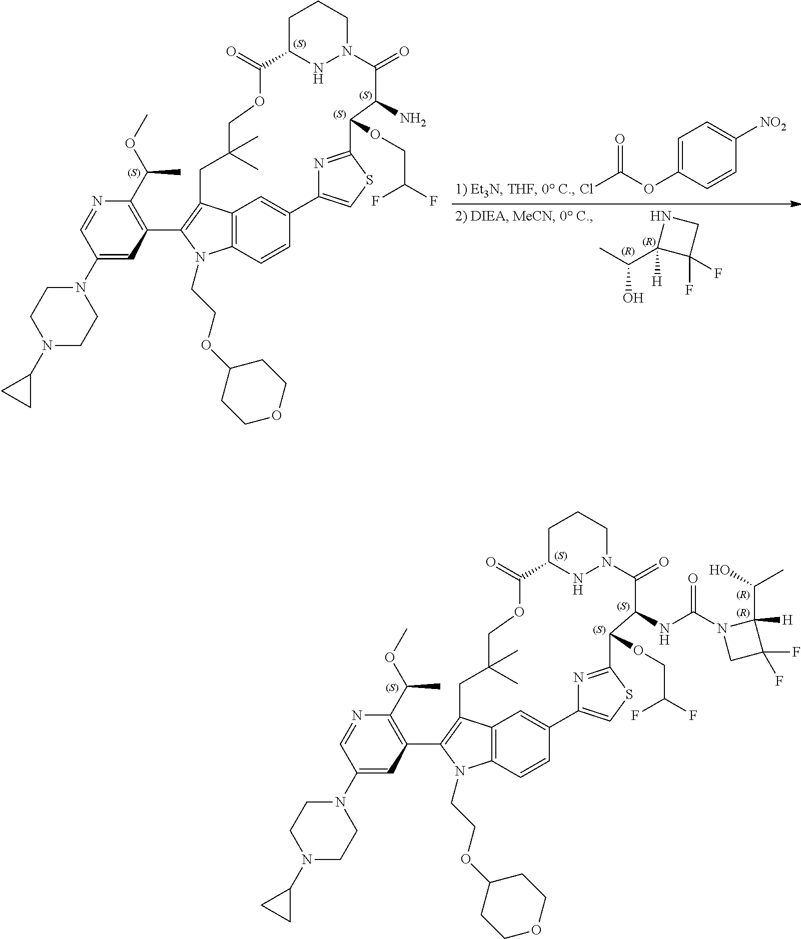

### Step 1

To a stirred solution of ethyl (2R)-2-hydroxypropanoate (20 g, 169.302 mmol) and 1H-imidazole (23.05 g, 338.604 mmol) in CH₂Cl₂ (200 mL, 3146.120 mmol) was added TBSCl (38.28 g, 253.953 mmol) dropwise at room temperature under air atmosphere. The reaction was quenched with sat. aq. NH₄Cl at room temperature. The aqueous layer was extracted with EtOAc (2×200 mL). The resulting mixture was concentrated under reduced pressure. The residue was purified by silica gel column chromatography, eluted with PE/EtOAc (9:1) to afford ethyl (2R)-2-[(tert-butyldimethylsilyl)oxy]propanoate (39 g, 99.12%) as a white oil. LCMS (ESI): m/z [M+H] calc'd for C₁₁H₂₄O₃Si 232.2; found 232.2.

### Step 2

To a stirred solution of ethyl (R)-2-((tert-butyldimethylsilyl)oxy) propanoate (19 g, 81.757 mmol) and CaCl₂) (18.15 g, 163.514 mmol) in THF (200 mL) was added NaBH₄ (12.37 g, 327.028 mmol) dropwise at 0° C. to room temperature under air atmosphere. The reaction was quenched with MeOH at 0° C. The precipitated solids were collected by filtration and washed with MeOH (3×50 mL). The resulting mixture was concentrated under reduced pressure. The residue was purified by silica gel column chromatography, eluted with PE/EtOAc (1:1) to afford (2R)-2-[(tert-butyldimethylsilyl)oxy]propan-1-ol (9.4 g, 60.40%) as a white oil. LCMS (ESI): m/z [M+H] calc'd for C₉H₂₂O₂Si 190.1; found 190.1.

### Step 3

A solution of (2R)-2-[(tert-butyldimethylsilyl)oxy]propan-1-ol (9.4 g, 49.381 mmol) and Dess-Martin (83.78 g, 197.524 mmol) in CH₂Cl₂ (300 mL) was stirred for 1 h at room temperature under air atmosphere. The new spot was identified by TLC. The residue was neutralized to pH 7 with sat. aq. NaHCO₃. The precipitated solids were collected by filtration and washed with CH₂Cl₂ (3×50 mL). The aqueous layer was extracted with CH₂Cl₂ (2×50 mL). The resulting mixture was concentrated under reduced pressure. The residue was purified by silica gel column chromatography, eluted with PE/EtOAc (1:1) to afford (2R)-2-[(tert-butyldimethylsilyl)oxy]propanal (5.413 g, 58.20%) as a yellow oil. LCMS (ESI): m/z [M+H] calc'd for C₉H₂₀O₂Si 188.1; found 188.1.

### Step 4

To a stirred solution of (2R)-2-[(tert-butyldimethylsilyl)oxy]propanal (5.413 g, 28.740 mmol) and (S)-2-methylpropane-2-sulfinamide (3.83 g, 31.614 mmol) in CH₂Cl₂ (100 mL) was added Ti(OEt)₄ (26.22 g, 114.960 mmol) dropwise at room temperature under air atmosphere. The residue was washed with H₂O (2×20 mL). The aqueous layer was extracted with CH₂Cl₂ (2×50 mL). The residue was purified by silica gel column chromatography, eluted with PE/EtOAc (5:1) to afford(S)—N-[(1E,2R)-2-[(tert-butyldimethylsilyl)oxy]propylidene]-2-methylpropane-2-sulfinamide (3.61 g, 43.09%) as a brown oil. LCMS (ESI): m/z [M+H] calc'd for C₁₃H₂₉NO₂SSi 291.2; found 291.2.

### Step 5

To a stirred solution of(S)—N-[(1E,2R)-2-[(tert-butyldimethylsilyl)oxy]propylidene]-2-methylpropane-2-sulfinamide (3.61 g, 12.383 mmol) and Rh(PPh₃)₃Cl (0.34 g, 0.371 mmol) in THF (50 mL) was added ZnEt₂ (3.06 g, 24.766 mmol) dropwise at −10° C. under air atmosphere. To the above mixture was added methyl 2-bromo-2,2-difluoroacetate (7.02 g, 37.149 mmol) dropwise over 2 min at −10° C. The resulting mixture was stirred for additional 1 h at −10° C. The mixture was acidified to pH 7 with conc. HCl. The reaction was quenched with sat. aq. NH₄Cl at room temperature. The aqueous layer was extracted with EtOAc (2×20 mL). The resulting mixture was concentrated under reduced pressure. The residue was purified by silica gel column chromatography, eluted with PE/EtOAc (1:1) to afford methyl (3R,4R)-4-[(tert-butyldimethylsilyl)oxy]-2,2-difluoro-3{[(S)-2-methylpropane-2-sulfinyl]amino}pentanoate (1.75 g, 35.19%) as a brown oil. LCMS (ESI): m/z [M+H] calc'd for C₁₆H₃₃F₂NO₄SSi 401.2; found 401.2.

### Step 6

To a stirred solution of methyl (3R,4R)-4-[(tert-butyldimethylsilyl)oxy]-2,2-difluoro-3-{[(S)-2-methylpropane-2-sulfinyl]amino}pentanoate (10 mg, 0.025 mmol) and CaCl₂) (0.97 g, 8.716 mmol) in THF (10 mL) was added NaBH₄ (0.66 g, 17.432 mmol) dropwise at room temperature under air atmosphere. The reaction was quenched by the addition of MeOH (20 mL) at 0° C. The precipitated solids were collected by filtration and washed with MeOH (3×10 mL). The resulting mixture was concentrated under reduced pressure. The residue was purified by silica gel column chromatography, eluted with PE/EtOAc (1:1) to afford(S)—N-[(3R,4R)-4-[(tert-butyldimethylsilyl)oxy]-2,2-difluoro-1-hydroxypentan-3-yl]-2-methylpropane-2-sulfinamide (1.13 g, 69.41%) as brown oil. LCMS (ESI): m/z [M+H] calc'd for C₁₅H₃₃F₂NO₃SSi 373.2; found 373.2.

### Step 7

A solution of(S)—N-[(3R,4R)-4-[(tert-butyldimethylsilyl)oxy]-2,2-difluoro-1-hydroxypentan-3-yl]-2-methylpropane-2-sulfinamide (500 mg, 1.338 mmol) and NaH (64.24 mg, 2.676 mmol) in THF (5 mL) was stirred for 30 min at room temperature under air atmosphere. To the above mixture was added TsCl (382.74 mg, 2.007 mmol) dropwise over 1 h at room temperature. To the above mixture was added NaH (64.24 mg, 2.676 mmol) dropwise over 2 min at room temperature. The resulting mixture was stirred for additional 36 h at room temperature. The reaction was quenched with sat. aq. NH₄Cl at room temperature. The residue was washed with H₂O (2×2 mL). The aqueous layer was extracted with EtOAc (2×2 mL). The resulting mixture was concentrated under reduced pressure. The residue was purified by silica gel column chromatography, eluted with PE/EtOAc (6:1) to afford (2R)-2-[(1R)-1-[(tert-butyldimethylsilyl)oxy]ethyl]-3,3-difluoro-1-[(S)-2-methylpropane-2-sulfinyl]azetidine (304 mg, 63.88%) as a yellow oil. LCMS (ESI): m/z [M+H] calc'd for C₁₅H₃₁F₂NO₂SSi 355.2; found 355.2.

### Step 8

A solution of (2R)-2-[(1R)-1-[(tert-butyldimethylsilyl)oxy]ethyl]-3,3-difluoro-1-[(S)-2-methylpropane-2-sulfinyl]azetidine (280 mg, 0.787 mmol) and TBAF (2 mL) in THF (2 mL) was stirred for 1 h at room temperature under air atmosphere. The residue was washed with H₂O (2×2 mL). The aqueous layer was extracted with EtOAc (2×2 mL). The residue was purified by silica gel column chromatography, eluted with PE/EtOAc (6:1) to afford (1R)-1-[(2R)-3,3-difluoro-1-[(S)-2-methylpropane-2-sulfinyl]azetidin-2-yl]ethanol (150 mg, 78.94%) as a yellow solid. LCMS (ESI): m/z [M+H] calc'd for C₉H₁₇F₂NO₂S 241.1; found 241.1.

### Step 9

A solution of (1R)-1-[(2R)-3,3-difluoro-1-[(S)-2-methylpropane-2-sulfinyl]azetidin-2-yl]ethanol (145 mg, 0.601 mmol) and HCl (g) in MeOH (2 mL, 65.826 mmol) in MeOH (2 mL) was stirred for 1 h at room temperature under air atmosphere. The resulting mixture was concentrated under reduced pressure to afford (1R)-1-[(2R)-3,3-difluoroazetidin-2-yl]ethanol (150 mg, crude) as a yellow solid. The crude product (150 mg, crude) was used in the next step directly without further purification. LCMS (ESI): m/z [M+H] calc'd for C₄₈H₆₄F₂N₈O₇S 934.5; found 934.5.

### Step 10

To a stirred solution of (6³S,3S,4S,Z)-4-amino-12-(5-(4-cyclopropylpiperazin-1-yl)-2-((S)-1-methoxyethyl)pyridin-3-yl)-3-(2,2-difluoroethoxy)-10,10-dimethyl-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (100 mg, 0.107 mmol) and Et₃N (32.46 mg, 0.321 mmol) in THF (4 mL) was added 4-nitrophenyl carbonochloridate (25.86 mg, 0.128 mmol) dropwise at 0° C. under an air atmosphere. To the above mixture was added (1R)-1-[(2R)-3,3-difluoroazetidin-2-yl]ethanol (29.33 mg, 0.214 mmol) and DIPEA (41.46 mg, 0.321 mmol) in MeCN (8 mL) dropwise over 2 min at 0° C. The resulting mixture was stirred for an additional 2 h at 0° C. The residue was washed with H₂O (2×5 mL). The aqueous layer was extracted with EtOAc (2×5 mL). The resulting mixture was concentrated under reduced pressure. The residue was purified by Prep-TLC to afford (2R)—N-((6³S,3S,4S,Z)-12-(5-(4-cyclopropylpiperazin-1-yl)-2-((S)-1-methoxyethyl)pyridin-3-yl)-3-(2,2-difluoroethoxy)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3,3-difluoro-2-((R)-1-hydroxyethyl) azetidine-1-carboxamide (15.2 mg, 12.94%) as a white solid. LCMS (ESI): m/z [M+H] calc'd for C₅₄H₇₁F₄N₉O₉S 1097.5; found 1097.5.

### Example 11: Synthesis of (1R,2S,3R)—N-((3S,4S,Z)-12-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-5,7-dioxo-3-(2-oxa-6-azaspiro[3.3]heptan-6-yl)-1¹H-8-oxa-6²,6³-diaza-2(4,2)-thiazola-1(5,3)-indola-6 (3,1)-bicyclo[3.1.1]heptanacycloundecaphane-4-yl)-2-methyl-3-(oxazol-4-yl) cyclopropane-1-carboxamide

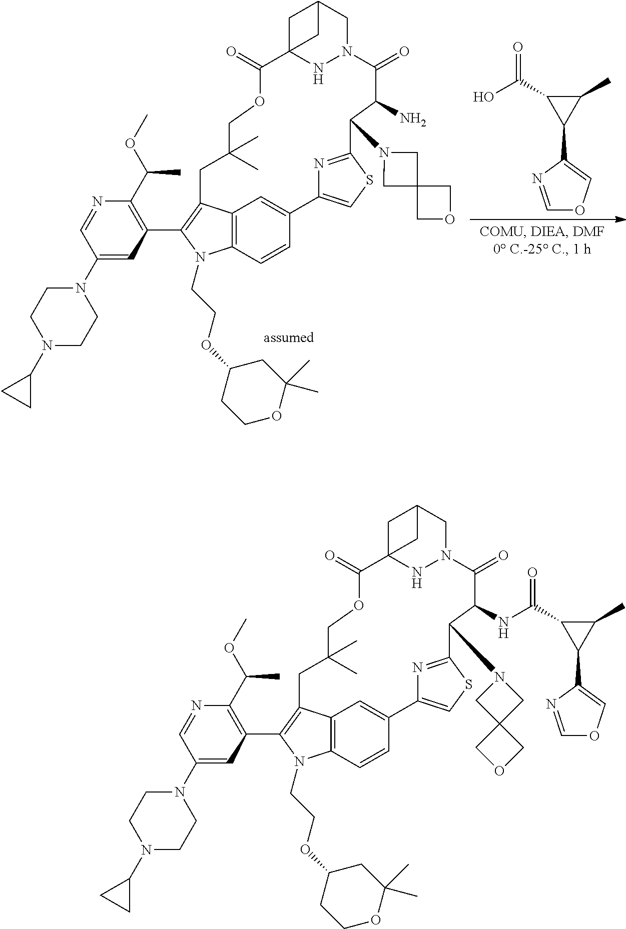

### Step 1

To a stirred solution of (3S,4S,Z)-4-amino-12-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-3-(2-oxa-6-azaspiro[3.3]heptan-6-yl)-1¹H-8-oxa-6²,6³-diaza-2(4,2)-thiazola-1(5,3)-indola-6 (3,1)-bicyclo[3.1.1]heptanacycloundecaphane-5,7-dione (120 mg, 0.121 mmol) and (1R,2S,3R)-2-methyl-3-(oxazol-4-yl) cyclopropane-1-carboxylic acid (24.26 mg, 0.145 mmol) in DMF (2 mL) were added DIPEA (156.30 mg, 1.210 mmol) and COMU (51.79 mg, 0.121 mmol) in portions at 0° C. The resulting mixture was stirred for 1 h at 25° C. The mixture was diluted with H₂O. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by reversed-phase flash chromatography to afford (1R,2S,3R)—N-((3S,4S,Z)-1²-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-5,7-dioxo-3-(2-oxa-6-azaspiro[3.3]heptan-6-yl)-1¹H-8-oxa-6²,6³-diaza-2(4,2)-thiazola-1(5,3)-indola-6 (3,1)-bicyclo[3.1.1]heptanacycloundecaphane-4-yl)-2-methyl-3-(oxazol-4-yl)cyclopropane-1-carboxamide (41 mg, 28% yield) as a white solid. LCMS (ESI): m/z [M+H] calc'd for C₆₂H₈₀N₁₀O₉S 1140.58; found 1140.58.

### Example 12: Synthesis of (2R)—N-((3S,4S,Z)-12-(5-(4-cyclopropylpiperazin-1-yl)-2-((S)-1-methoxyethyl)pyridin-3-yl)-1¹-(2-(((S)-2,2-dimethyltetrahydro-2H-pyran-4-yl)oxy)ethyl)-10,10-dimethyl-5,7-dioxo-3-(2-oxa-6-azaspiro[3.3]heptan-6-yl)-1¹H-8-oxa-6²,6³-diaza-2(4,2)-oxazola-1(5,3)-indola-6 (3,1)-bicyclo[3.1.1]heptanacycloundecaphane-4-yl)-2-methylazetidine-1-carboxamide

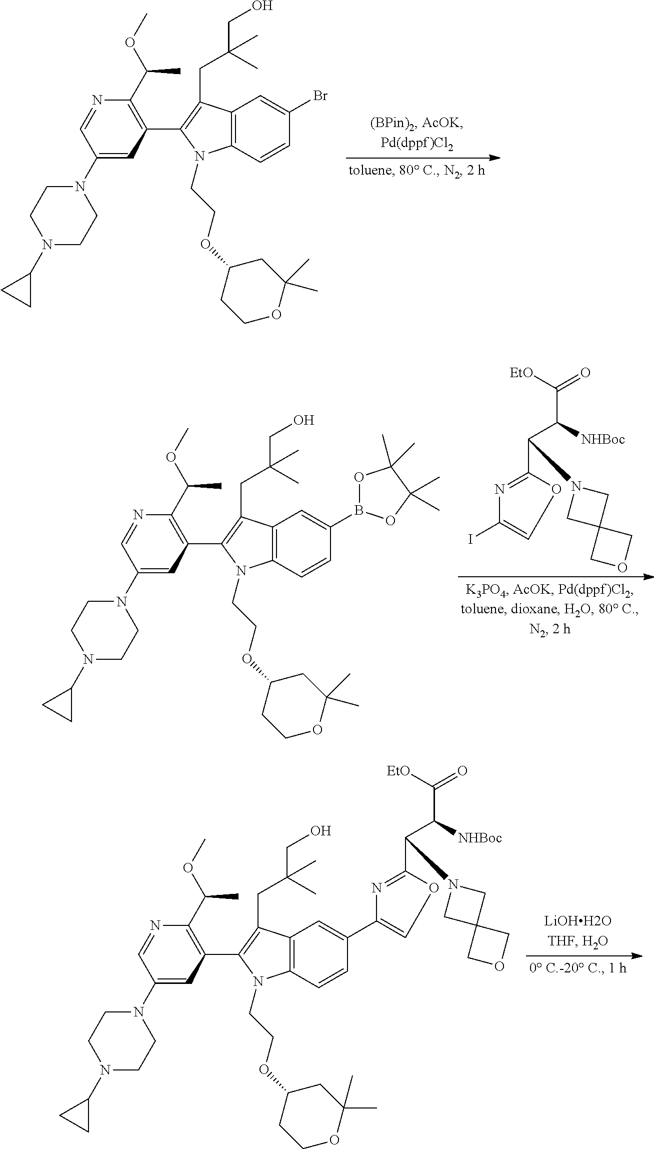

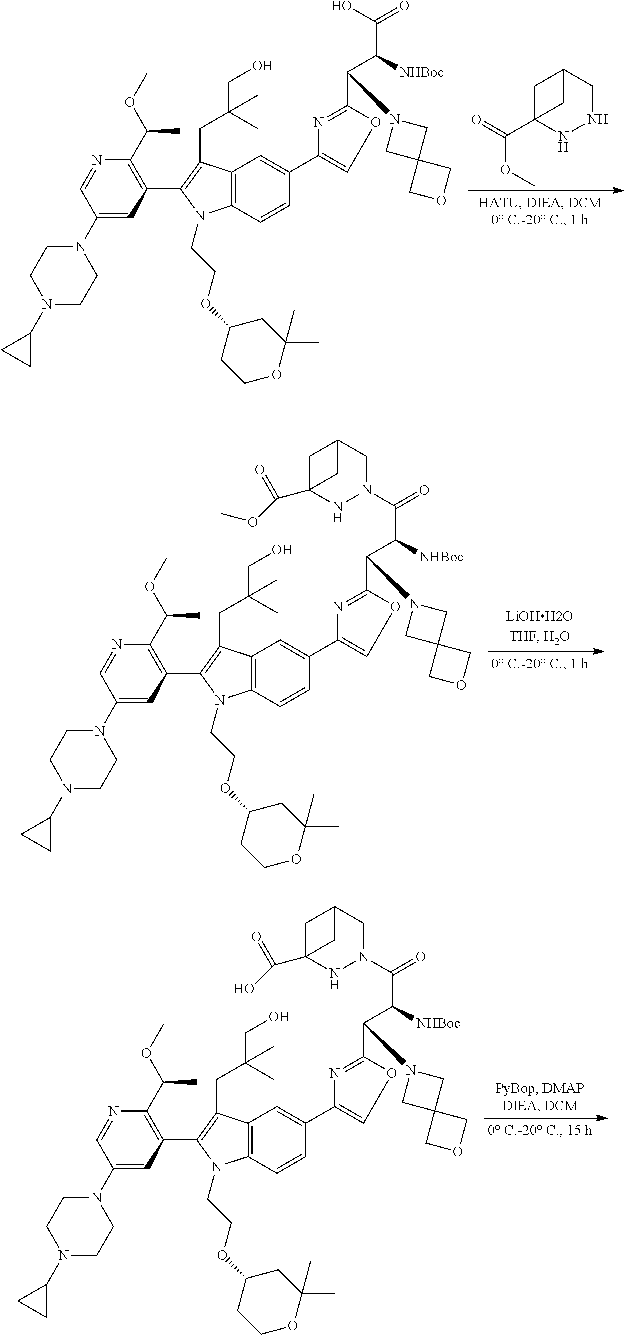

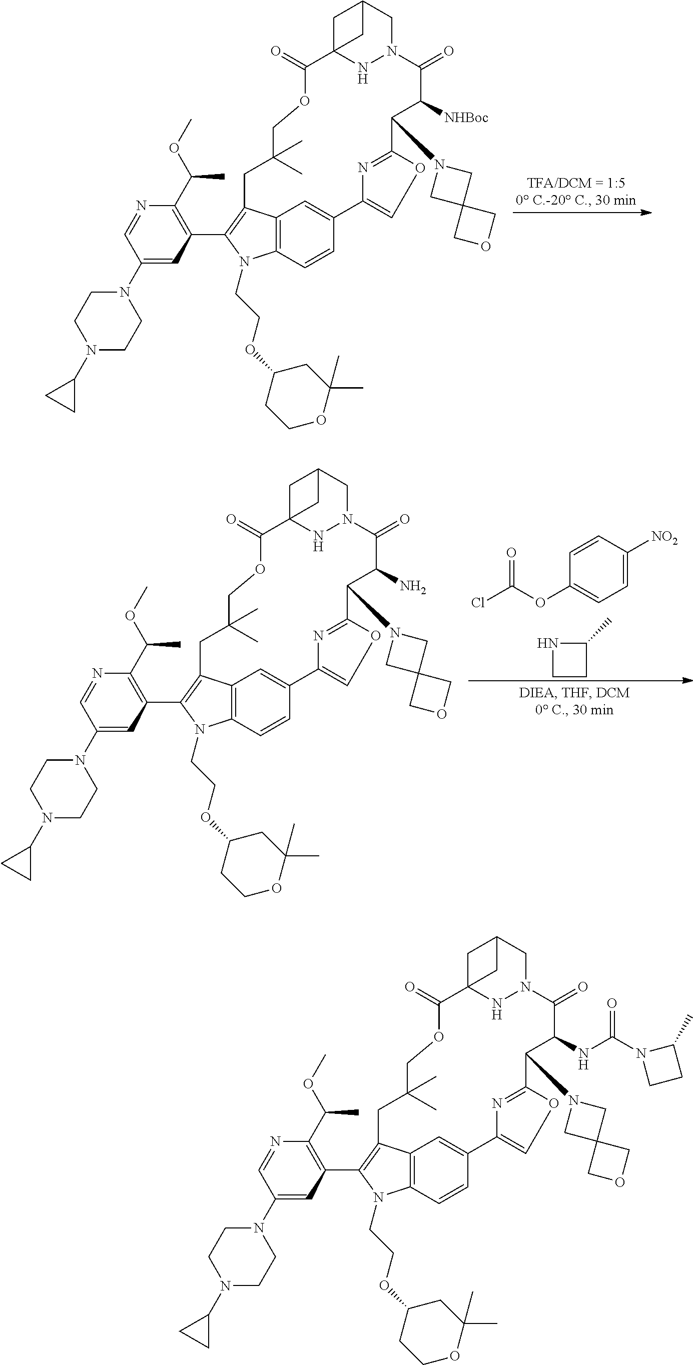

### Step 1

A solution of 3-(5-bromo-2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-1H-indol-3-yl)-2,2-dimethylpropan-1-ol (2 g, 2.866 mmol), 4,4,4′,4′,5,5,5′,5′-octamethyl-2,2′-bi (1,3,2-dioxaborolane) (1.46 g, 5.732 mmol), AcOK (0.84 g, 8.598 mmol) and Pd(dppf)Cl₂. CH₂Cl₂ (233.50 mg, 0.287 mmol) in toluene (20 mL) was stirred for 2 h at 80° C. under a nitrogen atmosphere. The resulting mixture was concentrated under reduced pressure. The residue was purified by silica gel column chromatography, eluted with PE/EtOAc (1:4) to afford 3-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-5-(4,4,5,5-tetramethyl-1,3,2-dioxaborolan-2-yl)-1H-indol-3-yl)-2,2-dimethylpropan-1-ol (1.62 g, 76%) as a brown solid. LCMS (ESI): m/z [M+H] calc'd for C₄₃H₆₅BN₄O₆ 744.50; found 744.50.

### Step 2

A solution of 3-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-5-(4,4,5,5-tetramethyl-1,3,2-dioxaborolan-2-yl)-1H-indol-3-yl)-2,2-dimethylpropan-1-ol (1.62 g, 2.181 mmol), ethyl (2S,3S)-2-((tert-butoxycarbonyl)amino)-3-(4-iodooxazol-2-yl)-3-(2-oxa-6-azaspiro[3.3]heptan-6-yl) propanoate (1.33 g, 2.617 mmol), K₃PO₄ (1.39 g, 6.543 mmol) and Pd(dppf)Cl₂· CH₂Cl₂ (177.65 mg, 0.218 mmol) in a solution of toluene (9 mL), dioxane (3 mL) and H₂O (3 mL) was stirred for 2 h at 80° C. under a nitrogen atmosphere. The resulting mixture was concentrated under reduced pressure. The residue was purified by silica gel column chromatography, eluted with PE/EtOAc (1:1) to afford ethyl (2S,3S)-2-((tert-butoxycarbonyl)amino)-3-(4-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-3-(3-hydroxy-2,2-dimethylpropyl)-1H-indol-5-yl) oxazol-2-yl)-3-(2-oxa-6-azaspiro[3.3]heptan-6-yl) propanoate (1.15 g, 52%) as a yellow solid. LCMS (ESI): m/z [M+H] calc'd for C₅₅H₇₉N₇O₁₀ 997.59; found 997.59.

### Step 3

To a stirred solution of ethyl (2S,3S)-2-((tert-butoxycarbonyl)amino)-3-(4-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-3-(3-hydroxy-2,2-dimethylpropyl)-1H-indol-5-yl) oxazol-2-yl)-3-(2-oxa-6-azaspiro[3.3]heptan-6-yl) propanoate (1.15 g, 1.152 mmol) in THF (7 mL) was added LiOH·H₂O (145.01 mg, 3.456 mmol) in H₂O (7 mL) dropwise at 0° C. under air atmosphere. The resulting mixture was stirred for 1 h at 20° C. under air atmosphere. The mixture was acidified to pH 5 with citric acid. The resulting mixture was extracted with EtOAc (3×20 mL). The combined organic layers were dried over anhydrous Na₂SO₄. After filtration, the filtrate was concentrated under reduced pressure. This resulted in (2S,3S)-2-((tert-butoxycarbonyl)amino)-3-(4-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-3-(3-hydroxy-2,2-dimethylpropyl)-1H-indol-5-yl) oxazol-2-yl)-3-(2-oxa-6-azaspiro[3.3]heptan-6-yl) propanoic acid (1.07 g, crude) as a yellow solid. The crude product was used in the next step directly without further purification. LCMS (ESI): m/z [M+H] calc'd for C₅₃H₇₅N₇O₁₀ 969.56; found 969.56.

### Step 4

To a stirred solution of (2S,3S)-2-((tert-butoxycarbonyl)amino)-3-(4-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-3-(3-hydroxy-2,2-dimethylpropyl)-1H-indol-5-yl) oxazol-2-yl)-3-(2-oxa-6-azaspiro[3.3]heptan-6-yl) propanoic acid, (1.07 g, 1.103 mmol) and methyl 2,3-diazabicyclo[3.1.1]heptane-1-carboxylate (516.74 mg, 3.309 mmol) in CH₂Cl₂ (15 mL) was added DIPEA (1.42 g, 11.030 mmol) and HATU (629.01 mg, 1.655 mmol) in portions at 0° C. The resulting mixture was stirred for additional 1 h at 20° C. The resulting mixture was concentrated under reduced pressure. The residue was purified by reversed-phase flash chromatography to afford methyl 3-((2S,3S)-2-((tert-butoxycarbonyl)amino)-3-(4-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-3-(3-hydroxy-2,2-dimethylpropyl)-1H-indol-5-yl) oxazol-2-yl)-3-(2-oxa-6-azaspiro[3.3]heptan-6-yl) propanoyl)-2,3-diazabicyclo[3.1.1]heptane-1-carboxylate (908 mg, 74.28%) as an off-white solid. LCMS (ESI): m/z [M+H] calc'd for C₆₀H₈₅N₉O₁₁ 1107.64; found 1107.64.

### Step 5

To a stirred solution of methyl 3-((2S,3S)-2-((tert-butoxycarbonyl)amino)-3-(4-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-3-(3-hydroxy-2,2-dimethylpropyl)-1H-indol-5-yl) oxazol-2-yl)-3-(2-oxa-6-azaspiro[3.3]heptan-6-yl) propanoyl)-2,3-diazabicyclo[3.1.1]heptane-1-carboxylate (900 mg, 0.821 mmol) in THF (5 mL) was added LiOH·H₂O (103.35 mg, 2.463 mmol) in H₂O (5 mL) dropwise at 0° C. under an air atmosphere. The resulting mixture was stirred for 1 h at 20° C. under air atmosphere. The mixture was acidified to pH 5 with citric acid. The resulting mixture was extracted with EtOAc (3×20 mL). The combined organic layers were dried over anhydrous Na₂SO₄. After filtration, the filtrate was concentrated under reduced pressure. This resulted in 3-((2S,3S)-2-((tert-butoxycarbonyl)amino)-3-(4-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-3-(3-hydroxy-2,2-dimethylpropyl)-1H-indol-5-yl) oxazol-2-yl)-3-(2-oxa-6-azaspiro[3.3]heptan-6-yl) propanoyl)-2,3-diazabicyclo[3.1.1]heptane-1-carboxylic acid (870 mg, crude) as an off-white solid. The crude product was used in the next step directly without further purification. LCMS (ESI): m/z [M+H] calc'd for C₅₉H₈₃N₉O₁₁ 1093.62; found 1093.62.

### Step 6

To a stirred solution of 3-((2S,3S)-2-((tert-butoxycarbonyl)amino)-3-(4-(2-(5-(4-cyclopropylpiperazin-1-yl)-2-((S)-1-methoxyethyl)pyridin-3-yl)-1-(2-(((S)-2,2-dimethyltetrahydro-2H-pyran-4-yl)oxy)ethyl)-3-(3-hydroxy-2,2-dimethylpropyl)-1H-indol-5-yl) oxazol-2-yl)-3-(2-oxa-6-azaspiro[3.3]heptan-6-yl) propanoyl)-2,3-diazabicyclo[3.1.1]heptane-1-carboxylic acid (846 mg, 0.773 mmol) in CH₂Cl₂ (423 mL) was added DIPEA (999.15 mg, 7.730 mmol) dropwise at 0° C. under air atmosphere. To the above mixture was added PyBOP (1206.89 mg, 2.319 mmol) and DMAP (94.44 mg, 0.773 mmol) in portions at 0° C. The resulting mixture was stirred for additional 15 h at 20° C. The resulting mixture was concentrated under reduced pressure. The residue was purified by reverse phase flash chromatography to afford tert-butyl ((3S,4S,Z)-12-(5-(4-cyclopropylpiperazin-1-yl)-2-((S)-1-methoxyethyl)pyridin-3-yl)-11-(2-(((S)-2,2-dimethyltetrahydro-2H-pyran-4-yl)oxy)ethyl)-10,10-dimethyl-5,7-dioxo-3-(2-oxa-6-azaspiro[3.3]heptan-6-yl)-11H-8-oxa-6²,6³-diaza-2(4,2)-oxazola-1(5,3)-indola-6 (3,1)-bicyclo[3.1.1]heptanacycloundecaphane-4-yl) carbamate (520 mg, 62.49%) as a white solid. LCMS (ESI): m/z [M+H] calc'd for C₅₉H₈₁N₉O₁₀ 1075.61; found 1075.61.

### Step 7

To a stirred solution of tert-butyl ((3S,4S,Z)-12-(5-(4-cyclopropylpiperazin-1-yl)-2-((S)-1-methoxyethyl)pyridin-3-yl)-11-(2-(((S)-2,2-dimethyltetrahydro-2H-pyran-4-yl)oxy)ethyl)-10,10-dimethyl-5,7-dioxo-3-(2-oxa-6-azaspiro[3.3]heptan-6-yl)-11H-8-oxa-62,63-diaza-2(4,2)-oxazola-1(5,3)-indola-6 (3,1)-bicyclo[3.1.1]heptanacycloundecaphane-4-yl) carbamate (520 mg, 0.483 mmol) in CH₂Cl₂ (26 mL) was added TFA (5.20 mL) dropwise at 0° C. under air atmosphere. The resulting mixture was stirred for 30 min at 20° C. under air atmosphere. The mixture was basified to pH 7 with sat. aq. NaHCO₃. The resulting mixture was extracted with CH₂Cl₂ (3×30 mL). The combined organic layers were dried over anhydrous Na₂SO₄. After filtration, the filtrate was concentrated under reduced pressure. This resulted in (3S,4S,Z)-4-amino-12-(5-(4-cyclopropylpiperazin-1-yl)-2-((S)-1-methoxyethyl)pyridin-3-yl)-11-(2-(((S)-2,2-dimethyltetrahydro-2H-pyran-4-yl)oxy)ethyl)-10,10-dimethyl-3-(2-oxa-6-azaspiro[3.3]heptan-6-yl)-11H-8-oxa-6²,6³-diaza-2(4,2)-oxazola-1(5,3)-indola-6 (3,1)-bicyclo[3.1.1]heptanacycloundecaphane-5,7-dione (460 mg, crude) as an off-white solid. The crude product was used in the next step directly without further purification. LCMS (ESI): m/z [M+H] calc'd for C₅₄H₇₃N₉O₈ 975.56; found 975.56.

### Step 8

To a stirred solution of (3S,4S,Z)-4-amino-12-(5-(4-cyclopropylpiperazin-1-yl)-2-((S)-1-methoxyethyl)pyridin-3-yl)-11-(2-(((S)-2,2-dimethyltetrahydro-2H-pyran-4-yl)oxy)ethyl)-10,10-dimethyl-3-(2-oxa-6-azaspiro[3.3]heptan-6-yl)-1¹H-8-oxa-6²,6³-diaza-2(4,2)-oxazola-1(5,3)-indola-6 (3,1)-bicyclo[3.1.1]heptanacycloundecaphane-5,7-dione (100 mg, 0.102 mmol) in THF (2 mL) was added DIPEA (132.39 mg, 1.020 mmol) dropwise at 0° C. under air atmosphere. To the above mixture was added 4-nitrophenyl carbonochloridate (61.94 mg, 0.306 mmol) in portions at 0° C. The resulting mixture was stirred for an additional 10 min at 0° C. The resulting mixture was used in the next step directly without further purification. To the above solution, (R)-2-methylazetidine (21.87 mg, 0.309 mmol) and DIPEA (132.49 mg, 1.030 mmol) in CH₂Cl₂ (2 mL) was added and dropwise at 0° C. under air atmosphere. The above mixture was added to the mixture of the last step dropwise at 0° C. The resulting mixture was stirred for additional 15 min at 0° C. The resulting mixture was concentrated under reduced pressure. The crude product (80 mg) was purified by Prep-HPLC to afford (2R)—N-((3S,4S,Z)-12-(5-(4-cyclopropylpiperazin-1-yl)-2-((S)-1-methoxyethyl)pyridin-3-yl)-11-(2-(((S)-2,2-dimethyltetrahydro-2H-pyran-4-yl)oxy)ethyl)-10,10-dimethyl-5,7-dioxo-3-(2-oxa-6-azaspiro[3.3]heptan-6-yl)-11H-8-oxa-62,63-diaza-2(4,2)-oxazola-1(5,3)-indola-6 (3,1)-bicyclo[3.1.1]heptanacycloundecaphane-4-yl)-2-methylazetidine-1-carboxamide (21.3 mg, 17.86%) as a white solid. LCMS (ESI): m/z [M+H] calc'd for C₅₉H₈₀N₁₀O₉ 1072.61; found 1072.61.

### Example 13: Synthesis of (1s,4R)—N-((6³S,3S,4S,Z)-1²-(5-(1-cyclopropylpiperidin-4-yl)-2-((S)-1-methoxyethyl)pyridin-3-yl)-10,10-dimethyl-5,7-dioxo-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-(methyl-d3)-2-oxabicyclo[2.1.1]hexane-4-d-1-carboxamide

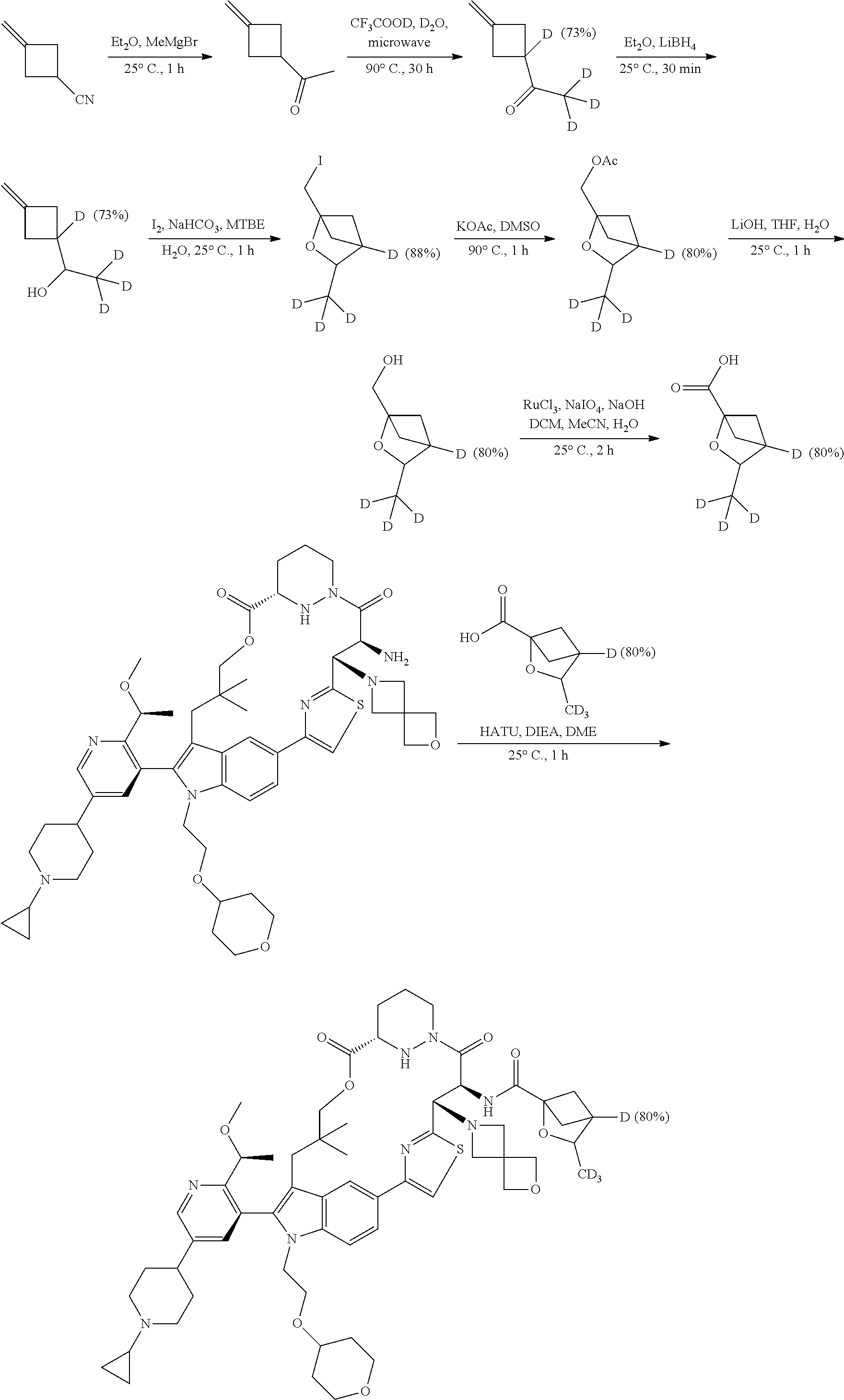

### Step 1

To a stirred solution of 3-methylidenecyclobutane-1-carbonitrile (10 g, 107.378 mmol) in Et₂O (100 mL) was added MeMgBr (89.48 mL, 268.445 mmol) dropwise at 0° C. The resulting mixture was stirred for 1 h at room temperature. The reaction was quenched by the addition of sat. aq. NH₄Cl (50 mL) at 0° C. The aqueous layer was extracted with Et₂O (2×50 mL). The resulting mixture was distilled under vacuum (70° C.). This resulted in 1-(3-methylidenecyclobutyl) ethanone (9.3 g, crude) as a colorless liquid. LCMS (ESI): m/z [M+H] calc'd for C₇H₁₀O 110.07; found 110.07.

### Step 2

To a stirred solution of 1-(3-methylidenecyclobutyl) ethanone (4 g, 36.312 mmol) in D₂O (40 mL) was added CF₃COOD (8.35 g, 72.624 mmol) in portions at 0° C. The final reaction mixture was irradiated with microwave radiation for 30 min at 90° C. The aqueous layer was extracted with Et₂O (3×20 mL). This resulted in 1-(3-methylenecyclobutyl-1-d) ethan-1-one-2,2,2-d3 (4 g, crude). The resulting mixture was used in the next step directly without further purification. LCMS (ESI): m/z [M+H] calc'd for C₇H₆D₃O 112.08; found 112.08.

### Step 3

To a stirred mixture of 1-(3-methylenecyclobutyl-1-d) ethan-1-one-2,2,2-d3 (4 g, 35.032 mmol) in Et₂O (60 mL) was added LiBH₄ (35.03 mL, 70.064 mmol) in portions at 0° C. The resulting mixture was stirred for 1 h at room temperature. The reaction was quenched by the addition of sat. aq. NH₄Cl (50 mL) at 0° C. The aqueous layer was extracted with EtOAc (2×50 mL). The resulting mixture was concentrated under reduced pressure. The residue was purified by reversed-phase flash chromatography to afford 1-(3-methylenecyclobutyl-1-d) ethan-2,2,2-d3-1-ol (512 mg, 12.58%) as a colorless liquid. LCMS (ESI): m/z [M+H] calc'd for C₇H₈D₃O 114.10; found 114.10.

### Step 4

To a stirred mixture of 1-(3-methylenecyclobutyl-1-d) ethan-2,2,2-d3-1-ol (520 mg, 4.475 mmol) in MTBE (5.2 mL) and H₂O (2.6 mL) was added NaHCO₃ (751.89 mg, 8.950 mmol) and I₂ (2271.69 mg, 8.950 mmol) in portions at room temperature. The resulting mixture was stirred for 2 h at room temperature. The reaction was monitored by TLC (pet. ether/EtOAc 5:1, Rf=0.5). The resulting mixture was diluted with H₂O (10 mL). The aqueous layer was extracted with EtOAc (3×10 mL). The resulting mixture was concentrated under reduced pressure. The residue was purified by Prep-HPLC to afford 1-(iodomethyl)-3-(methyl-d3)-2-oxabicyclo[2.1.1]hexane-4-d (280 mg, 25.84%) as a yellow solid. LCMS (ESI): m/z [M+H] calc'd for C₇H₇D₃IO 240.00; found 240.00.

### Step 5

To a stirred mixture of 1-(iodomethyl)-3-(methyl-d3)-2-oxabicyclo[2.1.1]hexane-4-d (300 mg, 1.239 mmol) in DMSO (3 mL) was added KOAc (182.43 mg, 1.859 mmol) in portions at room temperature. The resulting mixture was stirred for 1 h at 90° C. The resulting mixture was diluted with EtOAc (5 mL). The resulting mixture was washed with H₂O (3×10 mL). The resulting mixture was concentrated under reduced pressure. This resulted in (3-(methyl-d3)-2-oxabicyclo[2.1.1]hexan-1-yl-4-d)methyl acetate (200 mg, crude) as a brown, yellow liquid. LCMS (ESI): m/z [M+H] calc'd for C₉H₁₀D₃O₃ 172.11; found 172.11.

### Step 6

To a stirred mixture of (3-(methyl-d3)-2-oxabicyclo[2.1.1]hexan-1-yl-4-d)methyl acetate (190 mg, 1.091 mmol) in THF (1 mL) was added LiOH·H₂O (104.47 mg, 4.364 mmol) in H₂O (1 mL) dropwise at 0° C. The resulting mixture was stirred for 1 h at room temperature. The resulting mixture was diluted with H₂O (2 mL). The aqueous layer was extracted with CH₂Cl₂ (3×1 mL). The resulting mixture was concentrated under reduced pressure. This resulted in (3-(methyl-d3)-2-oxabicyclo[2.1.1]hexan-1-yl-4-d) methanol (150 mg, crude) as a yellow oil. LCMS (ESI): m/z [M+H] calc'd for C₇H₈D₃O₂ 130.09; found 130.09.

### Step 7

To a stirred mixture of (3-(methyl-d3)-2-oxabicyclo[2.1.1]hexan-1-yl-4-d) methanol (115 mg, 0.870 mmol) in CH₂Cl₂ (1 mL), MeCN (1 mL) and H₂O (3 mL) was added NaIO₄ (558.21 mg, 2.610 mmol), RuCl₃·H₂O (7.84 mg, 0.035 mmol) and NaOH (139.18 mg, 3.480 mmol) in portions at 0° C. The resulting mixture was stirred for 2 h at room temperature. The resulting mixture was diluted with H₂O (5 mL). The mixture was acidified to pH 5 with citric acid. The aqueous layer was extracted with CH₂Cl₂ (3×4 mL). The resulting mixture was concentrated under reduced pressure. This resulted in 3-(methyl-d3)-2-oxabicyclo[2.1.1]hexane-1-carboxylic-4-d acid (60 mg, crude) as a green oil. LCMS (ESI): m/z [M+H] calc'd for C₇H₆D₃O₃ 144.07; found 144.07.

### Step 8

A solution of (6³S,3S,4S,Z)-4-amino-12-(5-(1-cyclopropylpiperidin-4-yl)-2-((S)-1-methoxyethyl)pyridin-3-yl)-10,10-dimethyl-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (200 mg, 0.210 mmol) and 3-(methyl-d3)-2-oxabicyclo[2.1.1]hexane-1-carboxylic-4-d acid (46.10 mg, 0.315 mmol) in DMF (2 mL) was treated with DIPEA (271.74 mg, 2.100 mmol) for 5 min at 0° C. followed by the addition of HATU (95.93 mg, 0.252 mmol) in portions at 0° C. The resulting mixture was stirred for 1 h at room temperature. The resulting mixture was diluted with EtOAc (5 mL). The residue was washed with H₂O (3×5 mL). The resulting mixture was concentrated under reduced pressure. The crude product (100 mg) was purified by Prep-HPLC to afford (1 S,4R)—N-((6³S,3S,4S,Z)-12-(5-(1-cyclopropylpiperidin-4-yl)-2-((S)-1-methoxyethyl)pyridin-3-yl)-10,10-dimethyl-5,7-dioxo-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-(methyl-d3)-2-oxabicyclo[2.1.1]hexane-4-d-1-carboxamide (52.3 mg, 23.05%) as a white solid. LCMS (ESI): m/z [M+H] calc'd for C₅₉H₇₄D3N₈O₉S 1076.57; found 1076.57.

### Example 14: Synthesis of (2R,3S)—N-((6³S,3S,4S,Z)-12-(5-(1-cyclopropylazetidin-3-yl)-2-((S)-1-methoxyethyl)pyridin-3-yl)-10,10-dimethyl-5,7-dioxo-3-(2-oxa-6-azaspiro[3.3]heptan-6-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyloxetane-2-carboxamide

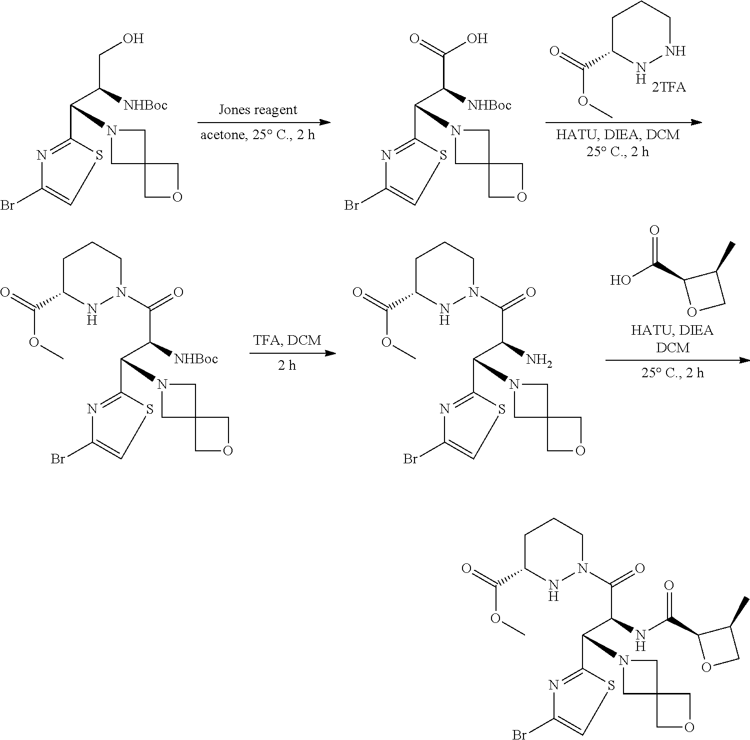

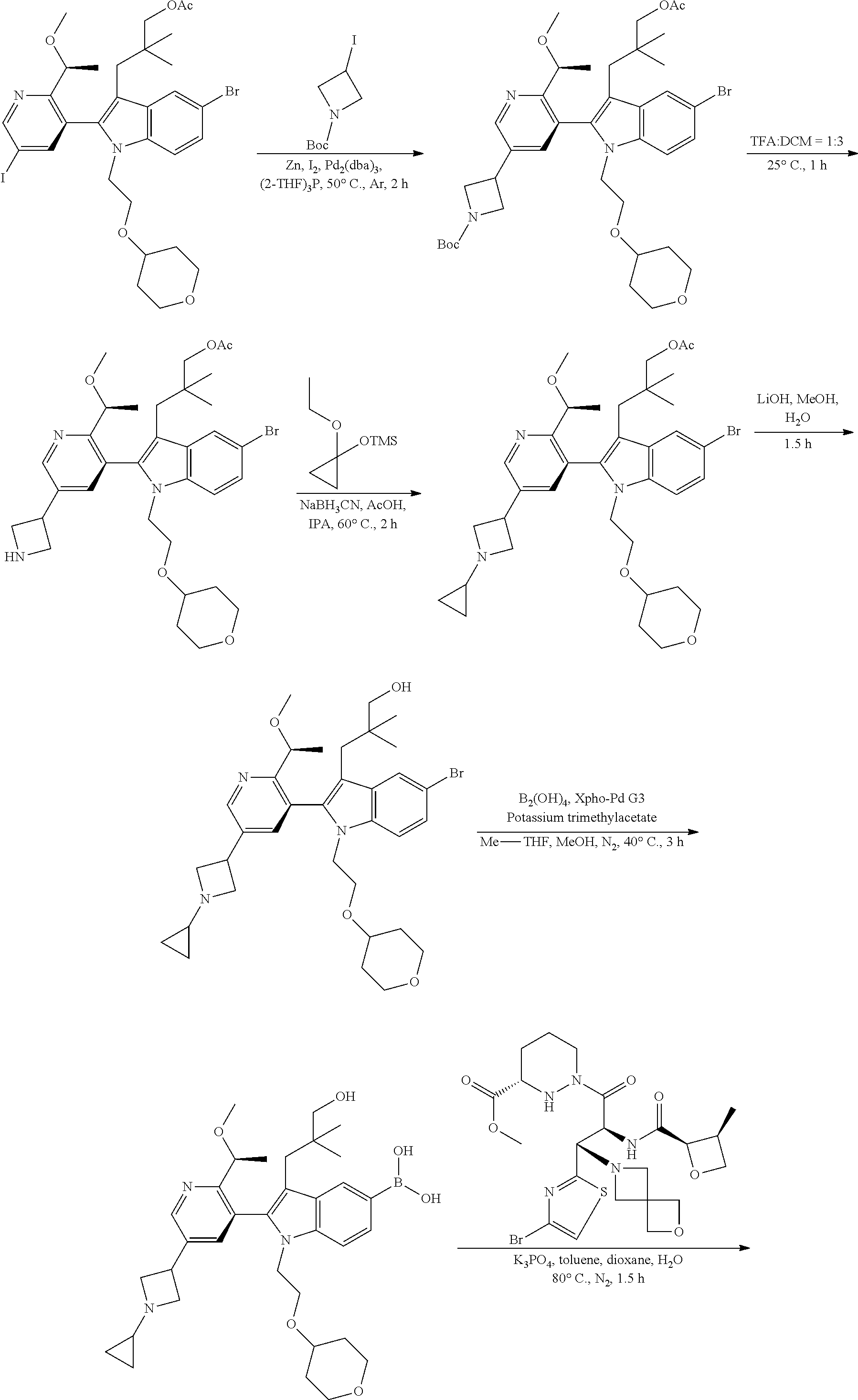

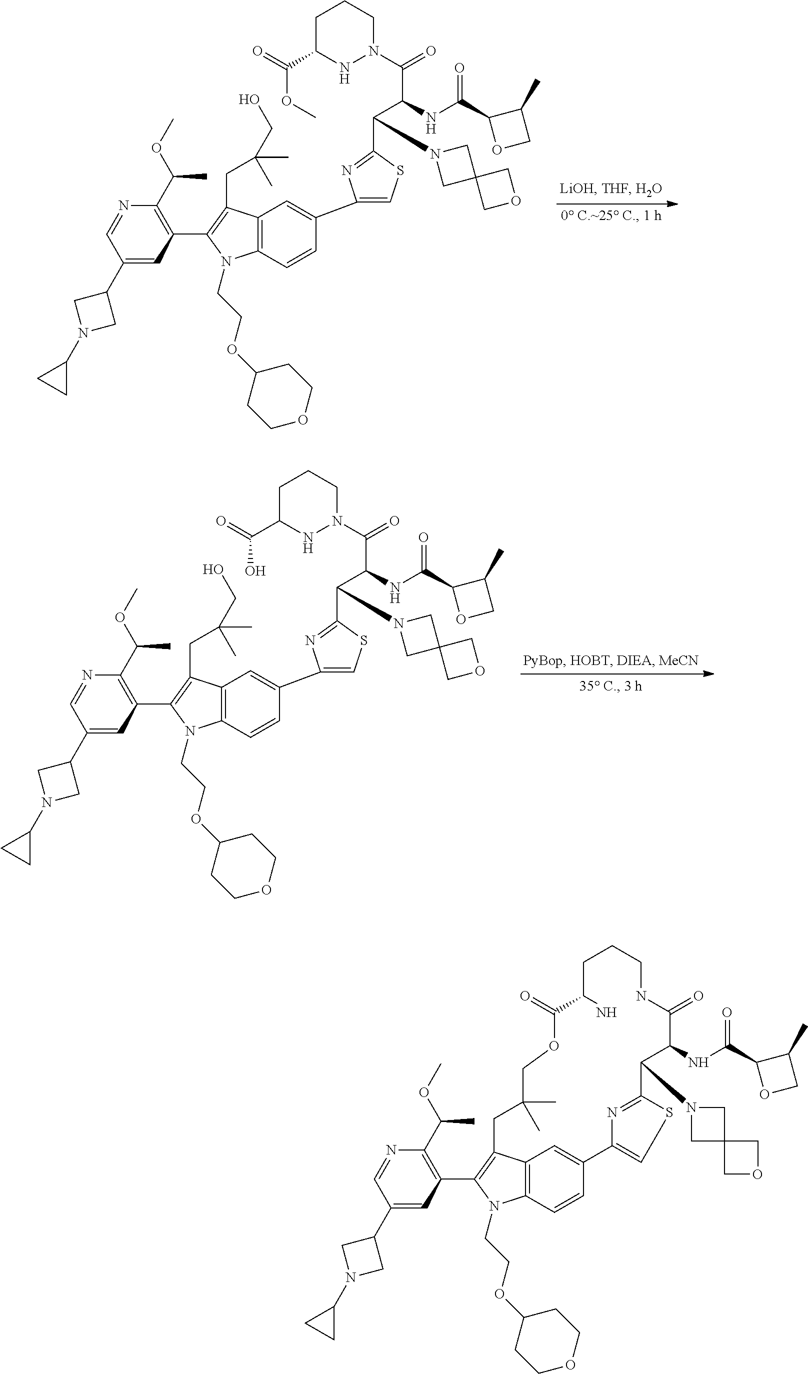

### Step 1

To a stirred solution of tert-butyl ((1 S,2S)-1-(4-bromothiazol-2-yl)-3-hydroxy-1-(2-oxa-6-azaspiro[3.3]heptan-6-yl) propan-2-yl) carbamate (5 g, 11.511 mmol) in acetone (50 mL) was added Jones reagent (17.27 mL, 34.533 mmol) dropwise at 0° C. The resulting mixture was stirred for 2 h at room temperature. The residue was purified by reverse-phase flash chromatography to afford (2S,3S)-3-(4-bromothiazol-2-yl)-2-((tert-butoxycarbonyl)amino)-3-(2-oxa-6-azaspiro[3.3]heptan-6-yl) propanoic acid (1.2 g, 23.25%) as a yellow solid. LCMS (ESI): m/z [M+H] calc'd for C₁₆H₂₂BrN₃O₅S 449.04; found 449.04.

### Step 2

To a stirred solution of (2S,3S)-3-(4-bromothiazol-2-yl)-2-((tert-butoxycarbonyl)amino)-3-(2-oxa-6-azaspiro[3.3]heptan-6-yl) propanoic acid (1.3 g, 2.900 mmol) in CH₂Cl₂ (13 mL) were added HATU (1.43 g, 3.770 mmol) and DIPEA (5.05 mL, 29.000 mmol) in portions at 0° C. The resulting mixture was stirred for 2 h at room temperature. The resulting mixture was diluted with H₂O (10 mL), extracted with CH₂Cl₂ (3×40 mL). The combined organic layers were washed with H₂O (3×40 mL), dried over anhydrous Na₂SO₄. After filtration, the filtrate was concentrated under reduced pressure. The residue was purified by silica gel column chromatography, eluted with PE/EtOAc (1:3) to afford methyl(S)-1-((2S,3S)-3-(4-bromothiazol-2-yl)-2-((tert-butoxycarbonyl)amino)-3-(2-oxa-6-azaspiro[3.3]heptan-6-yl) propanoyl) hexahydropyridazine-3-carboxylate (960 mg, 57.63%) as a white solid. LCMS (ESI): m/z [M+H] calc'd for C₂₂H₃₂BrN₅O₆S 575.12; found 575.12.

### Step 3

To a stirred solution of methyl(S)-1-((2S,3S)-3-(4-bromothiazol-2-yl)-2-((tert-butoxycarbonyl)amino)-3-(2-oxa-6-azaspiro[3.3]heptan-6-yl) propanoyl) hexahydropyridazine-3-carboxylate (1.43 g, 2.489 mmol) in CH₂Cl₂ (28.6 mL) was added TFA (5.7 mL) dropwise at 0° C. The resulting mixture was stirred for 1 h at 25° C. The reaction was quenched with sat. aq. NaHCO₃ at 0° C. The resulting mixture was extracted with CH₂Cl₂ (3×50 mL). The combined organic layers were washed with brine (2×30 mL), dried over anhydrous Na₂SO₄. After filtration, the filtrate was concentrated under reduced pressure. This resulted in methyl(S)-1-((2S,3S)-2-amino-3-(4-bromothiazol-2-yl)-3-(2-oxa-6-azaspiro[3.3]heptan-6-yl) propanoyl) hexahydropyridazine-3-carboxylate (1.3 g, crude) as a yellow green solid. The crude product was used in the next step directly without further purification. LCMS (ESI): m/z [M+H] calc'd for C₁₇H₂₄BrN₅O₄S 475.07; found 475.07.

### Step 4

To a stirred solution of methyl(S)-1-((2S,3S)-2-amino-3-(4-bromothiazol-2-yl)-3-(2-oxa-6-azaspiro[3.3]heptan-6-yl) propanoyl) hexahydropyridazine-3-carboxylate (1.25 g, 2.635 mmol) and (2R,3S)-3-methyloxetane-2-carboxylic acid (0.37 g, 3.162 mmol) in DMF (13 mL) were added DIPEA (3.41 g, 26.350 mmol) and HATU (1.30 g, 3.425 mmol) in portions at 0° C. The resulting mixture was stirred for 1 h at 25° C. The residue was purified by reverse-phase flash chromatography to afford methyl (S)-1-((2S,3S)-3-(4-bromothiazol-2-yl)-2-((2R,3S)-3-methyloxetane-2-carboxamido)-3-(2-oxa-6-azaspiro[3.3]heptan-6-yl) propanoyl) hexahydropyridazine-3-carboxylate (1.1 g, 69% yield) as a light yellow solid. LCMS (ESI): m/z [M+H] calc'd for C₂₂H₃₀BrN₅O₆S 573.11; found 573.11.

### Step 5

A mixture of Zn (7.33 g, 112.128 mmol) and |2 (1.42 g, 5.606 mmol) in DMF (50 mL) was stirred for 5 min at 50° C. under an argon atmosphere. To the above mixture was added tert-butyl 3-iodoazetidine-1-carboxylate (7.94 g, 28.032 mmol) in DMF (50 mL) dropwise at 0° C. The resulting mixture was stirred for an additional 2 h at 50° C. under an argon atmosphere. The resulting mixture was used in the following reaction directly. To a stirred mixture of(S)-3-(5-bromo-2-(5-iodo-2-(1-methoxyethyl)pyridin-3-yl)-1-(2-((tetrahydro-2H-pyran-4-yl)oxy)ethyl)-1H-indol-3-yl)-2,2-dimethylpropyl acetate (23 g, 14.016 mmol), Pd₂(dba)₃ (2.95 g, 3.224 mmol) and tris(furan-2-yl)phosphane (0.49 g, 2.102 mmol) in DMF (150 mL) was added the above resulting mixture dropwise at 0° C. under an argon atmosphere. The resulting mixture was stirred for 2 h at 50° C. under an argon atmosphere. The precipitated solids were collected by filtration and washed with EtOAc (3×50 mL). The resulting mixture was diluted with H₂O (500 mL), extracted with EtOAc (3×300 mL), dried over anhydrous Na₂SO₄. After filtration, the filtrate was concentrated under reduced pressure. The residue was purified by silica gel column chromatography, eluted with pet. ether/EtOAc (12:1) to afford tert-butyl(S)-3-(5-(3-(3-acetoxy-2,2-dimethylpropyl)-5-bromo-1-(2-((tetrahydro-2H-pyran-4-yl)oxy)ethyl)-1H-indol-2-yl)-6-(1-methoxyethyl)pyridin-3-yl) azetidine-1-carboxylate (16.2 g, 67.66%) as a brown oil. LCMS (ESI): m/z [M+H] calc'd for C₃₈H₅₂BrN₃O₇ 741.30; found 741.30.

### Step 6

A solution of tert-butyl(S)-3-(5-(3-(3-acetoxy-2,2-dimethylpropyl)-5-bromo-1-(2-((tetrahydro-2H-pyran-4-yl)oxy)ethyl)-1H-indol-2-yl)-6-(1-methoxyethyl)pyridin-3-yl) azetidine-1-carboxylate (8 g, 10.771 mmol) and TFA (20.00 mL) in CH₂Cl₂ (60 mL) was stirred for 1 h at room temperature. The mixture was neutralized to pH 7 with sat. aq. NaHCO₃. The resulting mixture was extracted with CH₂Cl₂ (3×80 mL). The combined organic layers were washed with H₂O (3×100 mL), dried over anhydrous Na₂SO₄. After filtration, the filtrate was concentrated under reduced pressure. This resulted in(S)-3-(2-(5-(azetidin-3-yl)-2-(1-methoxyethyl)pyridin-3-yl)-5-bromo-1-(2-((tetrahydro-2H-pyran-4-yl)oxy)ethyl)-1H-indol-3-yl)-2,2-dimethylpropyl acetate (6.5 g, crude) as a yellow solid. LCMS (ESI): m/z [M+H] calc'd for C₃₃H₄₄BrN₃O₅ 641.25; found 641.25.

### Step 7

To a stirred solution of(S)-3-(2-(5-(azetidin-3-yl)-2-(1-methoxyethyl)pyridin-3-yl)-5-bromo-1-(2-((tetrahydro-2H-pyran-4-yl)oxy)ethyl)-1H-indol-3-yl)-2,2-dimethylpropyl acetate (6.5 g, 10.115 mmol) and (1-ethoxycyclopropoxy)trimethylsilane (5.29 g, 30.345 mmol) in iPrOH (65 mL) were added NaBH₃CN (1.91 g, 30.345 mmol) and AcOH (1.74 mL, 30.345 mmol) in portions at room temperature. The resulting mixture was stirred for 1 h at 60° C. The reaction was quenched with sat. aq. NaHCO₃ at 0° C. The resulting mixture was extracted with EtOAc (3×100 mL). The combined organic layers were washed with H₂O (3×100 mL), dried over anhydrous Na₂SO₄. After filtration, the filtrate was concentrated under reduced pressure. The residue was purified by silica gel column chromatography, eluted with EtOAc/MeOH (4:1) to afford(S)-3-(5-bromo-2-(5-(1-cyclopropylazetidin-3-yl)-2-(1-methoxyethyl)pyridin-3-yl)-1-(2-((tetrahydro-2H-pyran-4-yl)oxy)ethyl)-1H-indol-3-yl)-2,2-dimethylpropyl acetate (4 g, 57.93%) as a yellow solid. LCMS (ESI): m/z [M+H] calc'd for C₃₆H₄₈BrN₃O₅ 683.28; found 683.28.

### Step 8

To a stirred solution of(S)-3-(5-bromo-2-(5-(1-cyclopropylazetidin-3-yl)-2-(1-methoxyethyl)pyridin-3-yl)-1-(2-((tetrahydro-2H-pyran-4-yl)oxy)ethyl)-1H-indol-3-yl)-2,2-dimethylpropyl acetate (4 g, 5.859 mmol) in MeOH (30 mL) and H₂O (10 mL) was added LiOH·H₂O (1.23 g, 29.295 mmol) in portions at 0° C. The resulting mixture was stirred for 1.5 h at room temperature. The mixture was acidified to pH 6 with citric acid. The resulting mixture was extracted with EtOAc (3×50 mL). The combined organic layers were washed with H₂O (3×60 mL), dried over anhydrous Na₂SO₄. After filtration, the filtrate was concentrated under reduced pressure. This resulted in(S)-3-(5-bromo-2-(5-(1-cyclopropylazetidin-3-yl)-2-(1-methoxyethyl)pyridin-3-yl)-1-(2-((tetrahydro-2H-pyran-4-yl)oxy)ethyl)-1H-indol-3-yl)-2,2-dimethylpropan-1-ol (3.85 g, crude) as a white solid. LCMS (ESI): m/z [M+H] calc'd for C₃₄H₄₆BrN₃O₄ 641.27; found 641.27.

### Step 9

To a stirred solution of(S)-3-(5-bromo-2-(5-(1-cyclopropylazetidin-3-yl)-2-(1-methoxyethyl)pyridin-3-yl)-1-(2-((tetrahydro-2H-pyran-4-yl)oxy)ethyl)-1H-indol-3-yl)-2,2-dimethylpropan-1-ol (1.24 g, 1.935 mmol) and tetrahydroxydiborane (0.21 g, 2.322 mmol) in 2-Me-THF (8 mL) and MeOH (4 mL) were added XPhos Pd 3^{rd} generation (0.05 g, 0.058 mmol), potassium 2,2-dimethylpropanoate (0.54 g, 3.870 mmol) in portions at 25° C. The resulting mixture was stirred for 3 h at 40° C. under a nitrogen atmosphere. The resulting mixture was filtered, the filter cake was washed with CH₂Cl₂ (3×30 mL). The filtrate was concentrated under reduced pressure. This resulted in(S)-(2-(5-(1-cyclopropylazetidin-3-yl)-2-(1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) boronic acid (1.35 g, crude) as a brown solid. LCMS (ESI): m/z [M+H] calc'd for C₃₄H₄₈BN₃O₆ 605.36; found 605.36.

### Step 10

To a solution of(S)-(2-(5-(1-cyclopropylazetidin-3-yl)-2-(1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) boronic acid (1.35 g, 2.229 mmol) and methyl(S)-1-((2S,3S)-3-(4-bromothiazol-2-yl)-2-((2R,3S)-3-methyloxetane-2-carboxamido)-3-(2-oxa-6-azaspiro[3.3]heptan-6-yl) propanoyl) hexahydropyridazine-3-carboxylate (1.28 g, 2.236 mmol) in toluene (15 mL), 1,4-dioxane (5 mL) and H₂O (5 mL) were added K₃PO₄ (1.42 g, 6.687 mmol), XPhos (0.21 g, 0.446 mmol) and XPhos Pd 3^{rd} generation (0.19 g, 0.223 mmol). After stirring for 2 h at 80° C. under a nitrogen atmosphere, the resulting mixture was concentrated under reduced pressure. The residue was purified by silica gel column chromatography to afford methyl(S)-1-((2S,3S)-3-(4-(2-(5-(1-cyclopropylazetidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-2-((2R,3S)-3-methyloxetane-2-carboxamido)-3-(2-oxa-6-azaspiro[3.3]heptan-6-yl) propanoyl) hexahydropyridazine-3-carboxylate (1.55 g, 62% yield) as a yellow solid. LCMS (ESI): m/z [M+H] calc'd for C₅₆H₇₆N₈O₁₀S 1052.54; found 1052.54.

### Step 11

To a stirred solution of methyl(S)-1-((2S,3S)-3-(4-(2-(5-(1-cyclopropylazetidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-2-((2R,3S)-3-methyloxetane-2-carboxamido)-3-(2-oxa-6-azaspiro[3.3]heptan-6-yl) propanoyl) hexahydropyridazine-3-carboxylate (1.55 g, 1.472 mmol) in THF (15 mL) were added LiOH·H₂O (0.11 g, 4.416 mmol) in H₂O (15 mL) dropwise at 0° C. The resulting mixture was stirred for 1 h at 25° C. The mixture was acidified to pH 5 with citric acid. The resulting mixture was extracted with EtOAc (3×50 mL). The combined organic layers were washed with brine (2×20 mL), dried over anhydrous Na₂SO₄. After filtration, the filtrate was concentrated under reduced pressure. This resulted in (S)-1-((2S,3S)-3-(4-(2-(5-(1-cyclopropylazetidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-2-((2R,3S)-3-methyloxetane-2-carboxamido)-3-(2-oxa-6-azaspiro[3.3]heptan-6-yl) propanoyl) hexahydropyridazine-3-carboxylic acid (1.6 g, crude) as a yellow solid. The crude product was used in the next step directly without further purification. LCMS (ESI): m/z [M+H] calc'd for C₅₅H₇₄N₈O₁₀S 1038.52; found 1038.52.

### Step 12

To a stirred solution of(S)-1-((2S,3S)-3-(4-(2-(5-(1-cyclopropylazetidin-3-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-2-((2R,3S)-3-methyloxetane-2-carboxamido)-3-(2-oxa-6-azaspiro[3.3]heptan-6-yl) propanoyl) hexahydropyridazine-3-carboxylic acid (1.5 g, 1.443 mmol) in MeCN (1000 mL) were added DIPEA (5.60 g, 43.290 mmol), HOBT (1.95 g, 14.430 mmol) and PyBOP (3.76 g, 7.215 mmol) in portions at 0° C. The resulting mixture was stirred for 3 h at 35° C. The resulting mixture was concentrated under reduced pressure. The resulting mixture was diluted with water (500 mL). The resulting mixture was extracted with EtOAc (3×500 mL). The combined organic layers were washed with brine (2×500 mL), dried over anhydrous Na₂SO₄. After filtration, the filtrate was concentrated under reduced pressure. The crude product was purified by Prep-HPLC to afford (2R,3S)—N-((6³S,3S,4S,Z)-12-(5-(1-cyclopropylazetidin-3-yl)-2-((S)-1-methoxyethyl)pyridin-3-yl)-10,10-dimethyl-5,7-dioxo-3-(2-oxa-6-azaspiro[3.3]heptan-6-yl)-11-(2-((tetrahydro-2H-pyran-4-yl)oxy)ethyl)-6¹,6²,6³,6⁴,6⁵,6⁶-hexahydro-11H-8-oxa-2(4,2)-thiazola-1(5,3)-indola-6(1,3)-pyridazinacycloundecaphane-4-yl)-3-methyloxetane-2-carboxamide (295.2 mg, 20% yield) as a white solid. LCMS (ESI): m/z [M+H] calc'd for C₅₅H₇₂N₈O₉S 1020.51; found 1020.51.

### Example 15: Synthesis of (1S,2R,3S)—N-((6³S,3S,4S,Z)-12-(5-(4-cyclopropylpiperazin-1-yl)-2-((S)-1-methoxyethyl)pyridin-3-yl)-10,10-dimethyl-5,7-dioxo-3-(pyrrolidin-1-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methyl-3-((R)-tetrahydrofuran-2-yl) cyclopropane-1-carboxamide

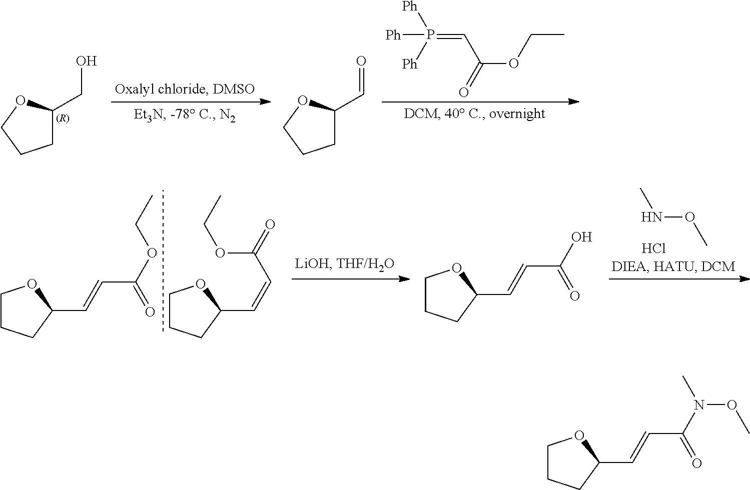

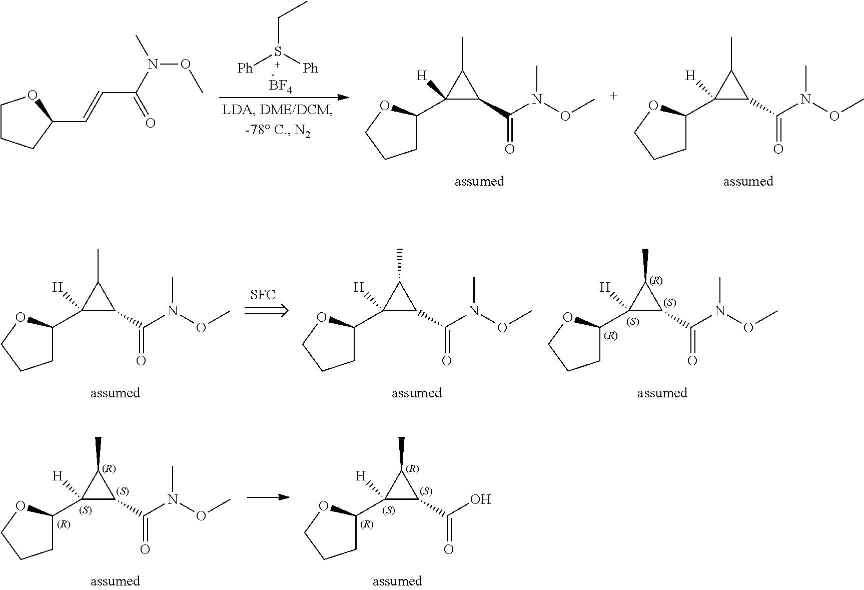

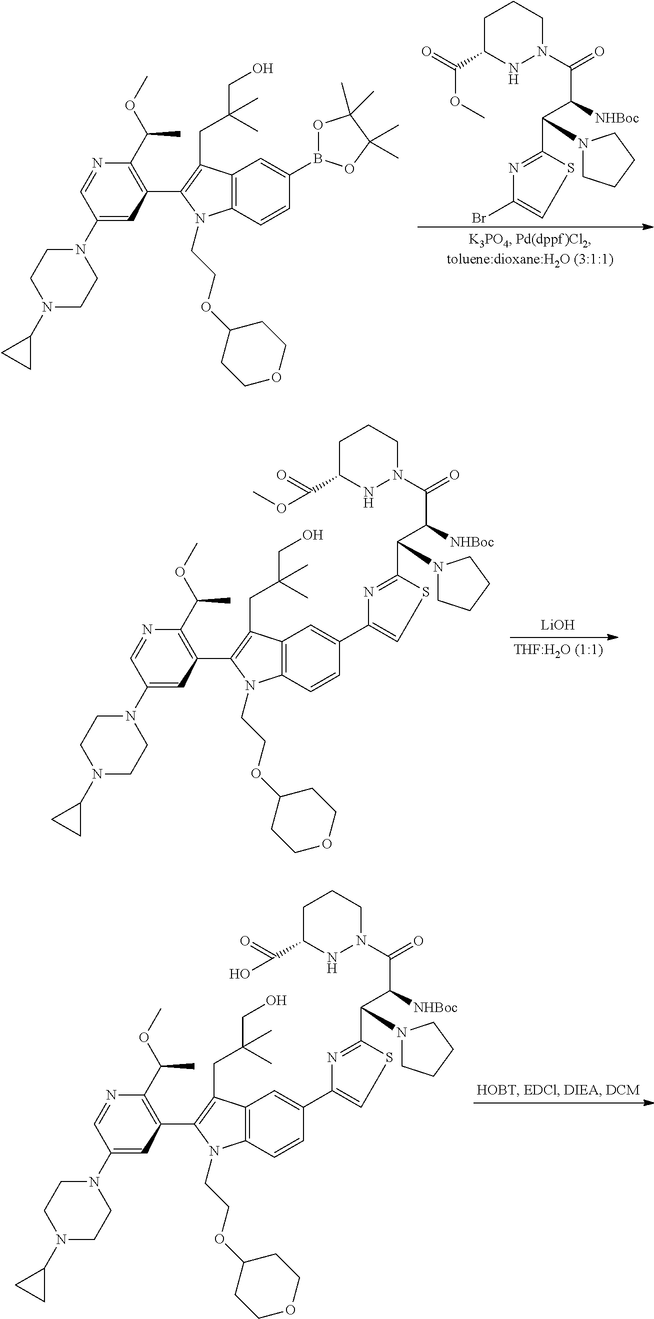

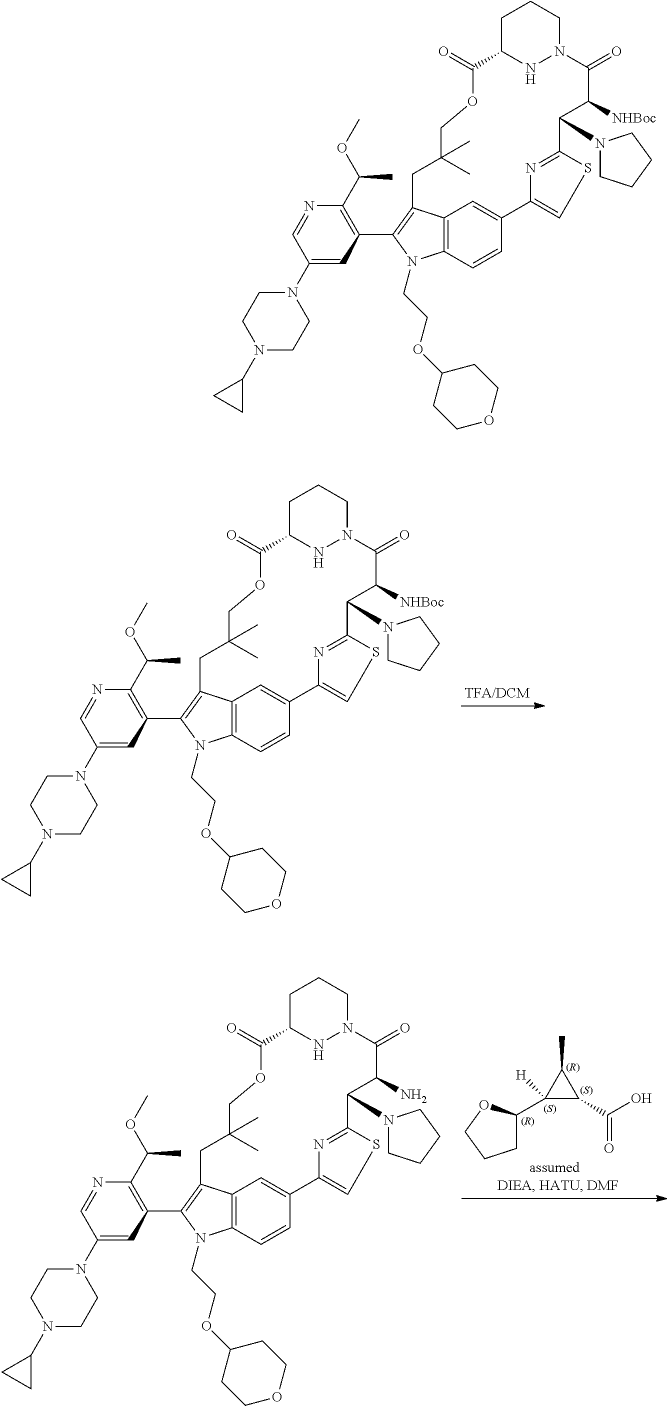

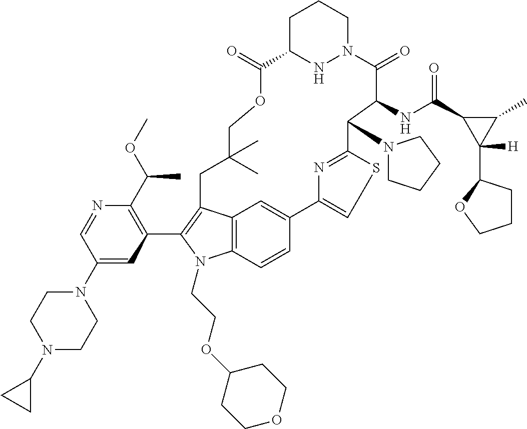

### Step 1

Into a 500-mL 3-necked-round-bottom flask purged and maintained with an inert atmosphere of nitrogen was placed oxalyl chloride (24.85 g, 195.824 mmol) and CH₂Cl₂ (200 mL). This was followed by the addition of DMSO (22.95 g, 293.736 mmol), (2R)-oxolan-2-ylmethanol (10 g, 97.912 mmol) and Et₃N (49.54 g, 489.560 mmol) in position at −78° C. The resulting mixture stirred for an additional 1 h at 25° C. The reaction was quenched by the addition of H₂O (200 mL) at 0° C. and extracted with CH₂Cl₂ (3×200 mL) and concentrated under reduced pressure. Removal of solvent resulted in 10 g (81.61%) (2R)-oxolane-2-carbaldehyde as a yellow liquid. LCMS (ESI): m/z [M+H] calc'd for C₅H₈O₂ 100.05; found 100.05.

### Step 2

Into a 500-mL 3-necked-round-bottom flask purged and maintained with an inert atmosphere of nitrogen, was placed (2R)-oxolane-2-carbaldehyde (10 g, 99.883 mmol), ethyl 2-(triphenyl-lambda5-phosphanylidene) acetate (34.80 g, 99.883 mmol) and CH₂Cl₂ (200 mL). The resulting mixture was stirred 12 h at 40° C. The reaction progress was monitored by TLC (pet. ether/EtOAc=5:1). The resulting mixture was washed with H₂O (200 mL), then extracted with CH₂Cl₂ (200 mL×3) and concentrated under reduced pressure. The residue was purified by silica gel column chromatography, eluted with pet. ether/EtOAc (6%) to afford (3E)-4-[(2R)-oxan-2-yl]but-3-en-2-one (6 g, 31.76%) as a yellow liquid. LCMS (ESI): m/z [M+H] calc'd for C₉H₁₄O₃ 170.09; found 170.09.

### Step 3

Into a 250-mL round-bottom flask purged and maintained with an inert atmosphere of nitrogen, was placed ethyl (2E)-3-[(2R)-oxolan-2-yl]prop-2-enoate (6 g, 35.251 mmol), THF (60 mL) and H₂O (60 mL). This was followed by the addition of LiOH (4.22 g, 176.255 mmol) in portions at 0° C. The resulting solution was stirred for 4 h at 25° C. The resulting mixture was concentrated under reduced pressure. The aqueous layer was employed to adjust the pH to 5 with 1 M aq. HCl. The resulting solution was extracted with EtOAc (3×100 mL). Removal of solvent resulted in (2E)-3-[(2R)-oxolan-2-yl]prop-2-enoic acid (5.4 g, 91.60%) as a yellow liquid. LCMS (ESI): m/z [M+H] calc'd for C₇H₁₀O₃ 142.06; found 142.06.

### Step 4

Into a 250-mL round-bottom flask purged and maintained with an inert atmosphere of nitrogen, was placed (2E)-3-[(2R)-oxolan-2-yl]prop-2-enoic acid (5.4 g, 36.939 mmol) and CH₂Cl₂ (100 mL). This was followed by the addition of DIPEA (23.87 g, 184.695 mmol), N, O-dimethylhydroxylamine (4.51 g, 73.878 mmol) and HATU (21.07 g, 55.409 mmol) at 0° C. The resulting solution was stirred for 1 h at 25° C. The reaction was then quenched by the addition of brine (100 mL). The resulting mixture was extracted with EtOAc (5×100 mL). The resulting mixture was concentrated under reduced pressure. The residue was purified by silica gel column chromatography, eluted with PE/EtOAc (40%) to afford (2E)-N-methoxy-N-methyl-3-[(2R)-oxolan-2-yl]prop-2-enamide (4.56 g, 58.70%) as a yellow liquid. LCMS (ESI): m/z [M+H] calc'd for C₉H₁₅NO₃ 185.11; found 185.11.

### Step 5

Into a 250-mL 3-necked-round-bottom flask purged and maintained with an inert atmosphere of nitrogen, was placed ethyldiphenylsulfonium tetrafluoroborate (22.39 g, 73.857 mmol), CH₂Cl₂ (20 mL) and DME (200 mL). To the resulting solution was added LDA (2 M in THF) (9.23 g, 86.166 mmol) dropwise at −78° C. under a nitrogen atmosphere over 30 min. To the above mixture was added (2E)-N-methoxy-N-methyl-3-[(2R)-oxolan-2-yl]prop-2-enamide (4.56 g, 24.619 mmol) in portions over 20 min at-78° C. The resulting mixture was stirred for an additional 2 h at 25° C. The reaction was quenched by the addition of aq. NH₄Cl (200 mL) at 0° C., then extracted with EtOAc (5×200 mL) and concentrated under reduced pressure. The residue was purified by reverse-phase flash chromatography to afford the product (1 g, 17.24% yield) as yellow liquid and V-1400-5H (560 mg, 9.25% yield) as yellow liquid. LCMS (ESI): m/z [M+H] calc'd for C₇H₁₀O₂ 126.1; found 126.1. The crude mixture was separated by chiral SFC to afford (1 S,2S,3S)—N-methoxy-N,2-dimethyl-3-((R)-tetrahydrofuran-2-yl) cyclopropane-1-carboxamide (160 mg, 42.1%) and (1 S,2R,3S)—N-methoxy-N,2-dimethyl-3-((R)-tetrahydrofuran-2-yl) cyclopropane-1-carboxamide (80 mg, 21.0%). LCMS (ESI): m/z [M+H] calc'd for C₁₁H₁₉NO₃ 213.14; found 213.14.

### Step 6

Into a 100-mL round-bottom flask purged and maintained with an inert atmosphere of nitrogen, was placed (1 S,2R,3S)—N-methoxy-N,2-dimethyl-3-((R)-tetrahydrofuran-2-yl) cyclopropane-1-carboxamide (110 mg, 0.516 mmol) and THF (5 mL). This was followed by the addition of t-BuOK (347.25 mg, 3.096 mmol) and H₂O (18.58 mg, 1.032 mmol) in portions at 0° C. The resulting solution was stirred for 12 h at 25° C. The resulting mixture was employed to adjust pH to 5 with 1 M aq. HCl and extracted with EtOAc (5×100 mL). Removal of solvent resulted in (1S,2R,3S)-2-methyl-3-((R)-tetrahydrofuran-2-yl) cyclopropane-1-carboxylic acid (70 mg, 71.76%) as a yellow liquid. LCMS (ESI): m/z [M+H] calc'd for C₉H₁₄O₃ 170.09; found 170.09.

### Step 7

To a stirred solution of(S)-3-(2-(5-(4-cyclopropylpiperazin-1-yl)-2-(1-methoxyethyl)pyridin-3-yl)-1-(2-((tetrahydro-2H-pyran-4-yl)oxy)ethyl)-5-(4,4,5,5-tetramethyl-1,3,2-dioxaborolan-2-yl)-1H-indol-3-yl)-2,2-dimethylpropan-1-ol (6 g, 8.371 mmol) and methyl(S)-1-((2,3S)-3-(4-bromothiazol-2-yl)-2-((tert-butoxycarbonyl)amino)-3-(pyrrolidin-1-yl) propanoyl) hexahydropyridazine-3-carboxylate (4.57 g, 8.371 mmol) in toluene (60 mL) and 1,4-dioxane (20 mL) and H₂O (20 mL) was added K₃PO₄ (4.44 g, 20.928 mmol) and Pd(dppf)Cl₂ (612.51 mg, 0.837 mmol) in portions at 75° C. under a nitrogen atmosphere. The desired product was detected by LCMS. The mixture was allowed to cool down to 25° C. The aqueous layer was extracted with EtOAc (2×20 mL). The resulting mixture was concentrated under reduced pressure. The residue was purified by silica gel column chromatography, eluted with PE/EtOAc (1:2) to afford methyl(S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(pyrrolidin-1-yl) propanoyl) hexahydropyridazine-3-carboxylate (7.3 g, 74.30%) as a brown solid. LCMS (ESI): m/z [M+H] calc'd for C₅₆H₈₁N₉O₉S 1055.59; found 1055.59.

### Step 8

To a stirred solution of methyl(S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(pyrrolidin-1-yl) propanoyl) hexahydropyridazine-3-carboxylate (7.3 g, 6.910 mmol) in THF (80 mL) and H₂O (80 mL) was added LiOH·H₂O (1.16 g, 27.640 mmol) in portions at 25° C. under air atmosphere. The aqueous layer was extracted with CH₂Cl₂ (2×50 mL). The resulting mixture was concentrated under reduced pressure. This resulted in(S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(pyrrolidin-1-yl) propanoyl) hexahydropyridazine-3-carboxylic acid (6.71 g, 83.84%) as a brown solid. LCMS (ESI): m/z [M+H] calc'd for C₅₅H₇₉N₉O₉S 1041.57; found 1041.57.

### Step 9

To a stirred solution of(S)-1-((2S,3S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2-((tetrahydro-2H-pyran-4-yl)oxy)ethyl)-1H-indol-5-yl) thiazol-2-yl)-3-(pyrrolidin-1-yl) propanoyl) hexahydropyridazine-3-carboxylic acid (6.7 g, 6.428 mmol) and DIPEA (33.23 g, 257.120 mmol) in CH₂Cl₂ (670 mL) was added EDCl (36.97 g, 192.840 mmol) and HOBT (8.69 g, 64.280 mmol) in portions at 25° C. under air atmosphere. The resulting mixture was washed with (1×300 mL) of 1 Naq. HCl. The resulting mixture was washed with 1×300 mL of aq. NaHCO₃. The resulting mixture was washed with H₂O (1×300 mL). The resulting mixture was concentrated under reduced pressure. The residue was purified by silica gel column chromatography, eluted with PE/EtOAc (1:2) to afford tert-butyl ((6³S,3S,4S,Z)-12-(5-(4-cyclopropylpiperazin-1-yl)-2-((S)-1-methoxyethyl)pyridin-3-yl)-10,10-dimethyl-5,7-dioxo-3-(pyrrolidin-1-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (4 g, 54.68%) as a brown solid. LCMS (ESI): m/z [M+H] calc'd for C₅₅H₇₇N₉O₈S 1023.56; found 1023.56.

### Step 10

To a stirred solution of tert-butyl ((6³S,3S,4S,Z)-12-(5-(4-cyclopropylpiperazin-1-yl)-2-((S)-1-methoxyethyl)pyridin-3-yl)-10,10-dimethyl-5,7-dioxo-3-(pyrrolidin-1-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl) carbamate (3.9 g, 3.807 mmol) in CH₂Cl₂ (40 mL) was added TFA (40 mL) dropwise at 0° C. under air atmosphere and stirred for 1 h at 25° C. The resulting mixture was concentrated under reduced pressure. The residue was dissolved in CH₂Cl₂ (50 mL). The mixture was neutralized to pH 8 with sat. aq. NaHCO₃. The aqueous layer was extracted with CH₂Cl₂ (3×40 mL). The resulting mixture was concentrated under reduced pressure. This resulted in (6³S,3S,4S,Z)-4-amino-1²-(5-(4-cyclopropylpiperazin-1-yl)-2-((S)-1-methoxyethyl)pyridin-3-yl)-10,10-dimethyl-3-(pyrrolidin-1-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (4.5 g, 89.52%) as a brown solid. LCMS (ESI): m/z [M+H] calc'd for C₅₀H₆₉N₉O₆S 923.51; found 923.51.

### Step 11

Into a 100 mL round-bottom flask were added (6³S,3S,4S,Z)-4-amino-1²-(5-(4-cyclopropylpiperazin-1-yl)-2-((S)-1-methoxyethyl)pyridin-3-yl)-10,10-dimethyl-3-(pyrrolidin-1-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (100 mg, 0.108 mmol) and DMF (3 mL) at room temperature. To the above mixture was added V-1400-7 (40 mg, 0.12 mmol) in portions over 1 min at room temperature. To the above mixture was added DIPEA (70 mg) in portions over 1 min at room temperature. To the above mixture was added HATU (50 mg) in portions over 1 min at room temperature. The resulting mixture was stirred for an additional 2 h at room temperature. The reaction was monitored by LCMS. The desired product was detected by LCMS. The resulting mixture was extracted with EtOAc (3×250 mL). The combined organic layers were washed with H₂O (3×10 mL), dried over anhydrous Na₂SO₄. After filtration, the filtrate was concentrated under reduced pressure. The crude product (160 mg) was purified by Prep-HPLC to afford (1S,2R,3S)—N-((6³S,3S,4S,Z)-1²-(5-(4-cyclopropylpiperazin-1-yl)-2-((S)-1-methoxyethyl)pyridin-3-yl)-10,10-dimethyl-5,7-dioxo-3-(pyrrolidin-1-yl)-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-methyl-3-((R)-tetrahydrofuran-2-yl) cyclopropane-1-carboxamide (18.3 mg, 15.71%) as a white solid. LCMS (ESI): m/z [M+H] calc'd for C₅₉H₈₁N₉O₈S 1075.59; found 1075.59.

### Example 16: Synthesis of in (2R,4R)—N-((6³S,3S,4S,Z)-12-(5-(4-cyclopropylpiperazin-1-yl)-2-((S)-1-methoxyethyl)pyridin-3-yl)-3-(3,3-dimethylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-4-methyloxetane-2-carboxamide and (2S,4R)—N-((6³S,3S,4S,Z)-12-(5-(4-cyclopropylpiperazin-1-yl)-2-((S)-1-methoxyethyl)pyridin-3-yl)-3-(3,3-dimethylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-4-methyloxetane-2-carboxamide

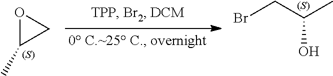

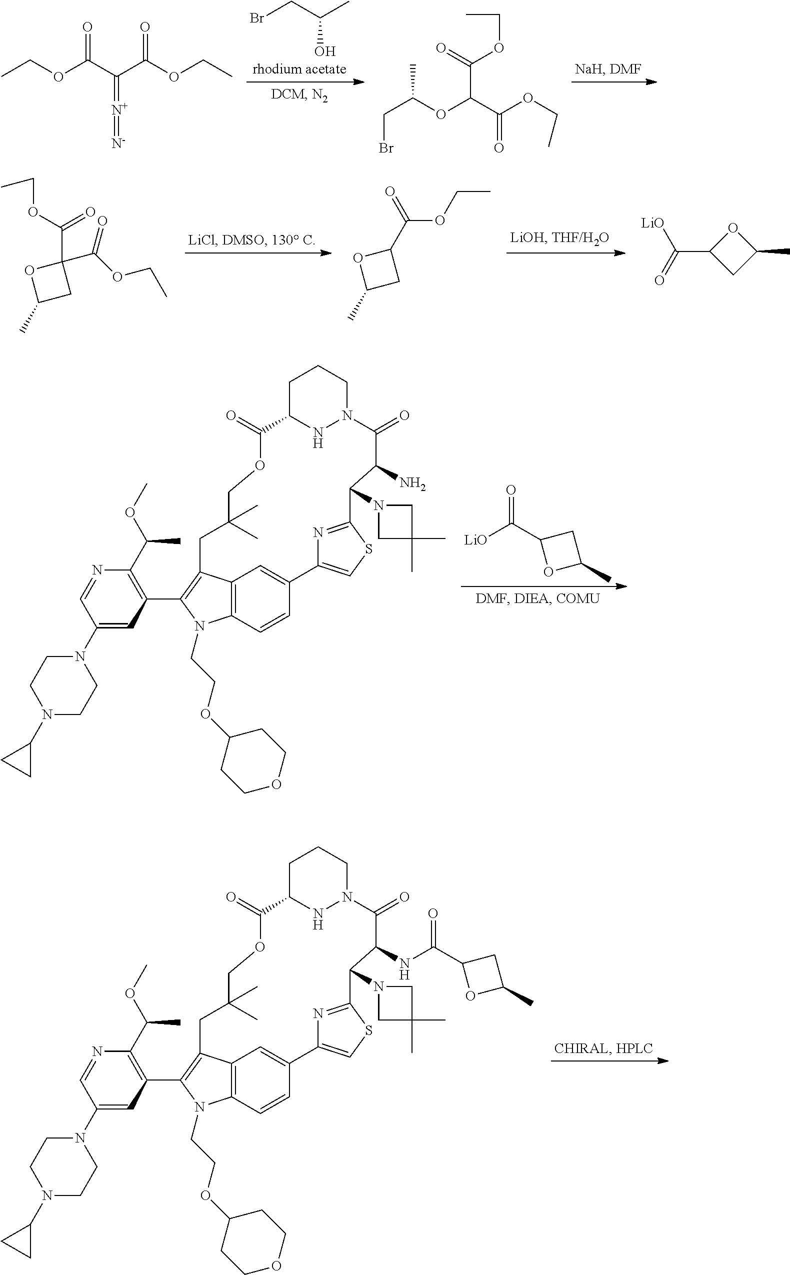

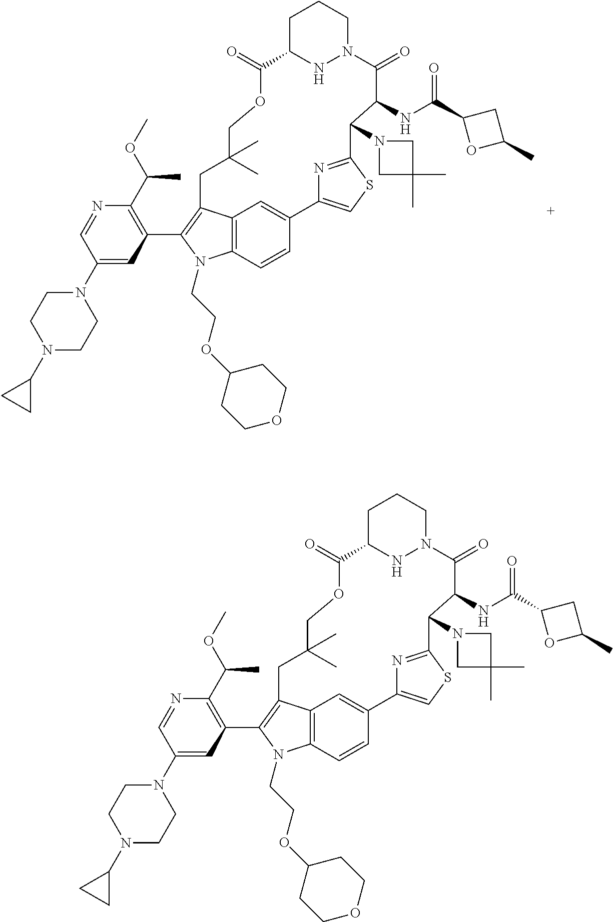

### Step 1

To a stirred solution of (−)-propylene oxide (30 g, 516.529 mmol) and TPP (31.71 g, 51.653 mmol) in CH₂Cl₂ was added Br₂ (82.55 g, 516.529 mmol) dropwise at 0° C. under air atmosphere. The resulting mixture was stirred for 8 h at 25° C. under an air atmosphere. The reaction was quenched with Na₂S₂O₃ at 0° C. The resulting mixture was washed with H₂O. The crude product was purified by distillation under 1 atm and the fraction was collected at 70° C. LCMS (ESI): m/z [M+H] calc'd for C₃H₇BrO 137.97; found 137.97.

### Step 2

To a stirred solution of 1-bromopropane-2-ol (2.30 g, 16.528 mmol) and rhodium acetate (0.23 g, 0.826 mmol) in CH₂Cl₂ was added 1,3-diethyl 2-diazopropanedioate (4 g, 21.486 mmol) dropwise at 0° C. under a nitrogen atmosphere. The resulting mixture was stirred for 4 h at 25° C. under an air atmosphere. The resulting mixture was filtered, the filter cake was washed with EtOAc. The filtrate was concentrated under reduced pressure. The residue was purified by silica gel column chromatography, eluted with PE/EtOAc to afford 1,3-diethyl 2-{[(2S)-1-bromopropan-2-yl]oxy}propanedioate as a colorless oil. LCMS (ESI): m/z [M+H] calc'd for C₁₀H₁₇BrO₅ 296.03; found 296.03.

### Step 3

A solution of diethyl(S)-2-((1-bromopropan-2-yl)oxy) malonate (5 g, 282.690 mmol) and NaH (10.18 g, 424.035 mmol) in 300 mL DMF was stirred for 4 h at 25° C. under air atmosphere. The reaction was monitored by LCMS. The reaction was quenched by the addition of sat. aq. NH₄Cl at 0° C. To the above mixture was added EtOAc. The resulting mixture was washed once with H₂O. The aqueous layer was extracted with EtOAc. The combined organic layers were washed with H₂O. The resulting mixture was concentrated under reduced pressure. The residue was purified by silica gel column chromatography, eluted with pet. ether/EtOAc (10:1) to afford diethyl(S)-4-methyloxetane-2,2-dicarboxylate (2 g, 40.90%) as a colorless oil. LCMS (ESI): m/z [M+H] calc'd for C₁₀H₁₆O₅ 216.10; found 216.10.

### Step 4

A solution of 2,2-diethyl (3S)-3-methyloxetane-2,2-dicarboxylate (2 g, 9.249 mmol) and LiCl (0.59 g, 13.873 mmol) in DMSO (20 mL) was stirred for 16 h at 130° C. under a nitrogen atmosphere. The reaction was monitored by LCMS. The mixture was allowed to cool down to 20° C. The reaction mixture was diluted with CH₂Cl₂ and washed with H₂O. The resulting mixture was concentrated under reduced pressure at 0° C. The crude product was used in the next step directly without further purification. LCMS (ESI): m/z [M+H] calc'd for C₇H₁₂O₃ 144.08; found 144.08.

### Step 5

A solution of ethyl (4S)-4-methyloxetane-2-carboxylate (1.5 g, 10.404 mmol) and LiOH—H₂O (0.65 g, 15.606 mmol) in THF (10 mL) and H₂O (10 mL) was stirred for 2 h at 20° C. under air atmosphere. The resulting mixture was concentrated under reduced pressure. The above mixture was freeze-drying to afford lithium (4S)-4-methyloxetane-2-carboxylate (1 g, 74%) as a brown solid. LCMS (ESI): m/z [M+H] calc'd for C₅H₇LiO₃ 122.06; found 122.06.

### Step 6

Into a 40 mL vial were added (6³S,3S,4S,Z)-4-amino-12-(5-(4-cyclopropylpiperazin-1-yl)-2-((S)-1-methoxyethyl)pyridin-3-yl)-3-(3,3-dimethylazetidin-1-yl)-10,10-dimethyl-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (80 mg, 0.085 mmol), DMF (2 mL), DIPEA (33.06 mg, 0.255 mmol), lithio-(4R)-4-methyloxetane-2-carboxylate (31.22 mg, 0.255 mmol) and COMU (54.77 mg, 0.128 mmol) at 0° C. The resulting mixture was stirred for 1 h at room temperature under an air atmosphere. The reaction was quenched with H₂O at room temperature. The resulting mixture was extracted with EtOAc (3×30 mL). The combined organic layers were washed with brine (1×50 mL), dried over anhydrous Na₂SO₄. After filtration, the filtrate was concentrated under reduced pressure. The crude product (80 mg) was purified by Prep-HPLC to afford (4R)—N-((6³S,3S,4S,Z)-1²-(5-(4-cyclopropylpiperazin-1-yl)-2-((S)-1-methoxyethyl)pyridin-3-yl)-3-(3,3-dimethylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-4-methyloxetane-2-carboxamide (25 mg) as a crude white solid. The crude product was purified by chiral-HPLC to afford (2R,4R)—N-((6³S,3S,4S,Z)-12-(5-(4-cyclopropylpiperazin-1-yl)-2-((S)-1-methoxyethyl)pyridin-3-yl)-3-(3,3-dimethylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-4-methyloxetane-2-carboxamide (6.1 mg, 6.76% yield) as a white solid and (2S,4R)—N-((6³S,3S,4S,Z)-12-(5-(4-cyclopropylpiperazin-1-yl)-2-((S)-1-methoxyethyl)pyridin-3-yl)-3-(3,3-dimethylazetidin-1-yl)-10,10-dimethyl-5,7-dioxo-1¹-(2-((tetrahydro-2H-pyran-4-yl)oxy)ethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-4-methyloxetane-2-carboxamide (6.4 mg, 7.09% yield) as a white solid. LCMS (ESI): m/z [M+H] calc'd for C₇H₁₀O₂ 126.1; found 126.1. LCMS (ESI): m/z [M+H] calc'd for C₅₆H₇₇N₉O₈S 1035.56; found 1035.56.

TABLE 5
Exemplary Compounds Prepared by
Methods of the Present Invention

| | Molecular | MS |
|---|---|---|
| Ex# | weight (g/mol) | Found |
| A1 | 904.103 | 904.5 |
| A2 | 988.269 | 988.4 |
| A3 | 1102.388 | 1102.65 |
| A4 | 1033.281 | 1033.6 |
| A5 | 1060.307 | 1060.6 |
| A6 | 1032.297 | 1032.65 |
| A7 | 807.993 | 808.45 |
| A8 | 820.004 | 820.4 |
| A9 | 1078.347 | 1078.9 |
| A10 | 1090.402 | 1090.9 |
| A11 | 1090.402 | 1090.9 |
| A12 | 1090.402 | 1090.9 |
| A13 | 1090.402 | 1091 |
| A14 | 1080.363 | 1080.6 |
| A15 | 1046.324 | 1046.7 |
| A16 | 1088.386 | 1088.9 |
| A17 | 876.068 | 876.5 |
| A18 | 1080.363 | 1080.6 |
| A19 | 1045.292 | 1045.55 |
| A20 | 1017.282 | 1017.55 |
| A21 | 1073.346 | 1073.6 |
| A22 | 976.258 | 976.4 |
| A23 | 1094.426 | 1094.8 |
| A24 | 1091.408 | 1091.9 |
| A25 | 1090.402 | 1090.9 |
| A26 | 1076.375 | 1076.8 |
| A27 | 1037.338 | 1038 |
| A28 | 1037.338 | 1038.1 |
| A29 | 1023.311 | 1024 |
| A30 | 1035.322 | 1036.1 |
| A31 | 823.992 | 824.4 |
| A32 | 1085.357 | 1085.5 |
| A33 | 1031.309 | 1031.6 |
| A34 | 1018.295 | 1018.4 |
| A35 | 852.071 | 852.3 |
| A36 | 918.027 | 918.25 |
| A37 | 839.991 | 840.25 |
| A38 | 1022.283 | 1022.55 |
| A39 | 1045.292 | 1045.5 |
| A40 | 1071.374 | 1071.65 |
| A41 | 911.153 | 911.45 |
| A42 | 879.141 | 879.3 |
| A43 | 1077.403 | 1077.55 |
| A44 | 1077.403 | 1077.65 |
| A45 | 1077.403 | 1077.55 |
| A46 | 1077.403 | 1077.6 |
| A47 | 1079.411 | 1079.4 |
| A48 | 894.108 | 894.4 |
| A49 | 908.135 | 908.45 |
| A50 | 1075.387 | 1075.5 |
| A51 | 1075.387 | 1075.5 |
| A52 | 903.163 | 903.35 |
| A53 | 862.041 | 862.5 |
| A54 | 837.975 | 838.35 |
| A55 | 833.987 | 834.4 |
| A56 | 849.986 | 850.4 |
| A57 | 1063.376 | 1063.55 |
| A58 | 1063.376 | 1063.55 |
| A59 | 1063.376 | 1063.6 |
| A60 | 1064.364 | 1064.55 |
| A61 | 1064.364 | 1064.5 |
| A62 | 940.177 | 940.5 |
| A63 | 1095.451 | 1095.6 |
| A64 | 1073.35 | 1073.65 |
| A65 | 859.041 | 859.45 |
| A66 | 1086.345 | 1086.5 |
| A67 | 1046.28 | 1046.5 |
| A68 | 1034.269 | 1034.7 |
| A69 | 1046.28 | 1046.6 |
| A70 | 934.104 | 934.4 |
| A71 | 906.094 | 906.5 |
| A72 | 920.121 | 920.5 |
| A73 | 980.242 | 980.5 |
| A74 | 980.242 | 980.5 |
| A75 | 859.041 | 859.3 |
| A76 | 1018.27 | 1018.65 |
| A77 | 1032.297 | 1032.65 |
| A78 | 990.241 | 990.6 |
| A79 | 1128.426 | 1128.55 |
| A80 | 887.095 | 887.5 |
| A81 | 875.084 | 875.45 |
| A82 | 863.073 | 863.3 |
| A83 | 891.152 | 891.25 |
| A84 | 874.052 | 874.25 |
| A85 | 849.986 | 850.35 |
| A86 | 863.935 | 864.45 |
| A87 | 821.976 | 822.5 |
| A88 | 777.967 | 778.45 |
| A89 | 1114.399 | 1114.7 |
| A90 | 1063.376 | 1063.65 |
| A91 | 940.177 | 940.5 |
| A92 | 1053.337 | 1053.5 |
| A93 | 1053.337 | 1053.6 |
| A94 | 1023.311 | 1023.65 |
| A95 | 1037.338 | 1037.55 |
| A96 | 1037.338 | 1037.6 |
| A97 | 1051.321 | 1051.6 |
| A98 | 1051.321 | 1051.6 |
| A99 | 1021.295 | 1021.4 |
| A100 | 1035.322 | 1035.55 |
| A101 | 1035.322 | 1035.65 |
| A102 | 1105.457 | 1105.7 |
| A103 | 1091.43 | 1091.4 |
| A104 | 1119.484 | 1119.65 |
| A105 | 1089.458 | 1089.7 |
| A106 | 1089.458 | 1089.7 |
| A107 | 1103.441 | 1103.6 |
| A108 | 1103.441 | 1103.65 |
| A109 | 1016.279 | 1016.45 |
| A110 | 1051.365 | 1051.8 |
| A111 | 905.179 | 905.5 |
| A112 | 893.168 | 893.35 |
| A113 | 875.946 | 876.25 |
| A114 | 904 | 904.45 |
| A115 | 836.003 | 836.4 |
| A116 | 1074.334 | 1074.55 |
| A117 | 1033.35 | 1033.55 |
| A118 | 1077.403 | 1077.7 |
| A119 | 1077.403 | 1077.7 |
| A120 | 1078.391 | 1078.65 |
| A121 | 1078.391 | 1078.6 |
| A122 | 1078.391 | 1078.6 |
| A123 | 1078.391 | 1078.6 |
| A124 | 1064.364 | 1064.6 |
| A125 | 1064.364 | 1064.65 |
| A126 | 1036.354 | 1036.5 |
| A127 | 1034.338 | 1034.6 |
| A128 | 926.15 | 926.4 |
| A129 | 997.273 | 997.5 |
| A130 | 1025.327 | 1025.55 |
| A131 | 1051.365 | 1051.65 |
| A132 | 995.257 | 995.55 |
| A133 | 1105.457 | 1105.75 |
| A134 | 1041.362 | 1042.1 |
| A135 | 1038.344 | 1039.2 |
| A136 | 916.011 | 916.5 |
| A137 | 954.204 | 954.35 |
| A138 | 1019.367 | 1019.5 |
| A139 | 1019.367 | 1019.55 |
| A140 | 979.302 | 979.6 |
| A141 | 1091.43 | 1091.4 |
| A142 | 1088.333 | 1088.45 |
| A143 | 881.113 | 881.35 |
| A144 | 919.162 | 919.45 |
| A145 | 919.162 | 919.45 |
| A146 | 894.083 | 894.55 |
| A147 | 954.204 | 954.4 |
| A148 | 954.204 | 954.45 |
| A149 | 968.231 | 968.45 |
| A150 | 968.231 | 968.5 |
| A151 | 994.269 | 994.6 |
| A152 | 994.269 | 994.6 |
| A153 | 1005.34 | 1005.45 |
| A154 | 1087.442 | 1087.55 |
| A155 | 1087.442 | 1087.5 |
| A156 | 822.089 | 822.35 |
| A157 | 895.11 | 895.45 |
| A158 | 932.079 | 932.35 |
| A159 | 920.068 | 920.3 |
| A160 | 922.093 | 922.4 |
| A161 | 908.11 | 908.55 |
| A162 | 906.119 | 906.45 |
| A163 | 1061.404 | 1061.45 |
| A164 | 1021.339 | 1021.5 |
| A165 | 1021.339 | 1021.5 |
| A166 | 1048.365 | 1048.7 |
| A167 | 1048.365 | 1048.55 |
| A168 | 980.242 | 980.45 |
| A169 | 994.269 | 994.55 |
| A170 | 1026.315 | 1026.45 |
| A171 | 1026.315 | 1026.4 |
| A172 | 1067.364 | 1067.5 |
| A173 | 1024.299 | 1024.45 |
| A174 | 1024.299 | 1024.45 |
| A175 | 1065.348 | 1065.5 |
| A176 | 1065.348 | 1065.5 |
| A177 | 1008.344 | 1008.5 |
| A178 | 1035.366 | 1035.55 |
| A179 | 1035.366 | 1035.6 |
| A180 | 1105.457 | 1105.6 |
| A181 | 1105.457 | 1105.55 |
| A182 | 1117.468 | 1117.7 |
| A183 | 1089.414 | 1089.65 |
| A184 | 1089.414 | 1089.65 |
| A185 | 1076.415 | 1076.7 |
| A186 | 1076.415 | 1076.7 |
| A187 | 1101.469 | 1101.6 |
| A188 | 1101.469 | 1101.75 |
| A189 | 1089.458 | 1089.7 |
| A190 | 1089.458 | 1089.7 |
| A191 | 1089.414 | 1089.55 |
| A192 | 1089.414 | 1089.45 |
| A193 | 978.23 | 978.55 |
| A194 | 866.098 | 866.3 |
| A195 | 909.137 | 909.45 |
| A196 | 1087.442 | 1087.55 |
| A197 | 1077.403 | 1077.65 |
| A198 | 1035.366 | 1035.6 |
| A199 | 1022.327 | 1022.6 |
| A200 | 1022.327 | 1022.6 |
| A201 | 914.139 | 914.4 |
| A202 | 914.139 | 914.4 |
| A203 | 1067.364 | 1067.55 |
| A204 | 1092.432 | 1092.65 |
| A205 | 1092.432 | 1092.6 |
| A206 | 952.232 | 952.6 |
| A207 | 1049.393 | 1049.7 |
| A208 | 1062.323 | 1062.5 |
| A209 | 1062.323 | 1062.45 |
| A210 | 834.1 | 834.4 |
| A211 | 1090.402 | 1090.5 |
| A212 | 1025.327 | 1025.45 |
| A213 | 1033.394 | 1033.6 |
| A214 | 1131.495 | 1131.6 |
| A215 | 1103.441 | 1103.7 |
| A216 | 1089.414 | 1089.5 |
| A217 | 1113.48 | 1113.6 |
| A218 | 1113.48 | 1113.6 |
| A219 | 1092.432 | 1092.6 |
| A220 | 1092.432 | 1092.7 |
| A221 | 1078.405 | 1078.6 |
| A222 | 1088.361 | 1088.65 |
| A223 | 1124.398 | 1124.7 |
| A224 | 1113.371 | 1113.6 |
| A225 | 906.119 | 906.45 |
| A226 | 1049.349 | 1049.55 |
| A227 | 1049.349 | 1049.5 |
| A228 | 1050.337 | 1050.55 |
| A229 | 1050.337 | 1050.55 |
| A230 | 966.215 | 966.45 |
| A231 | 966.215 | 966.45 |
| A232 | 1135.421 | 1135.55 |
| A233 | 882.097 | 882.3 |
| A234 | 928.166 | 928.45 |
| A235 | 928.166 | 928.45 |
| A236 | 1023.311 | 1023.45 |
| A237 | 1023.311 | 1023.6 |
| A238 | 912.167 | 912.45 |
| A239 | 938.205 | 938.5 |
| A240 | 1061.404 | 1061.5 |
| A241 | 1016.279 | 1016.5 |
| A242 | 1123.41 | 1123.6 |
| A243 | 1060.351 | 1060.55 |
| A244 | 1049.349 | 1049.45 |
| A245 | 926.194 | 926.4 |
| A246 | 952.232 | 952.3 |
| A247 | 1020.355 | 1020.5 |
| A248 | 1091.43 | 1091.6 |
| A249 | 1091.43 | 1091.6 |
| A250 | 1089.414 | 1089.5 |
| A251 | 1076.419 | 1076.5 |
| A252 | 1074.334 | 1074.5 |
| A253 | 1072.362 | 1072.6 |
| A254 | 964.243 | 964.3 |
| A255 | 867.086 | 867.3 |
| A256 | 905.135 | 905.5 |
| A257 | 1049.349 | 1049.45 |
| A258 | 924.178 | 924.45 |
| A259 | 938.205 | 938.55 |
| A260 | 1075.431 | 1075.55 |
| A261 | 1107.411 | 1107.6 |
| A262 | 1100.416 | 1100.6 |
| A263 | 1125.382 | 1125.45 |
| A264 | 1026.329 | 1026.8 |
| A265 | 934.095 | 934.25 |
| A266 | 908.135 | 908.4 |
| A267 | 1117.468 | 1117.65 |
| A268 | 1103.441 | 1103.5 |
| A269 | 1073.459 | 1073.6 |
| A270 | 1045.405 | 1045.55 |
| A271 | 1075.431 | 1075.55 |
| A272 | 1074.447 | 1074.6 |
| A273 | 1046.393 | 1046.5 |
| A274 | 1101.469 | 1101.55 |
| A275 | 1073.415 | 1073.55 |
| A276 | 1075.431 | 1075.6 |
| A277 | 1047.377 | 1047.45 |
| A278 | 866.054 | 866.35 |
| A279 | 964.174 | 964.5 |
| A280 | 1136.409 | 1136.45 |
| A281 | 1135.421 | 1135.55 |
| A282 | 1089.418 | 1089.4 |
| A283 | 904.069 | 904.35 |
| A284 | 891.152 | 891.45 |
| A285 | 893.124 | 893.55 |
| A286 | 878.109 | 878.4 |
| A287 | 880.081 | 880.25 |
| A288 | 1089.418 | 1089.5 |
| A289 | 1100.441 | 1100.4 |
| A290 | 1062.392 | 1062.4 |
| A291 | 1048.365 | 1048.5 |
| A292 | 1087.442 | 1087.55 |
| A293 | 914.12 | 914.25 |
| A294 | 920.146 | 920.35 |
| A295 | 1064.408 | 1064.6 |
| A296 | 1062.392 | 1062.45 |
| A297 | 1038.326 | 1038.5 |
| A298 | 924.222 | 924.5 |
| A299 | 961.287 | 961.45 |
| A300 | 947.26 | 947.4 |
| A301 | 892.092 | 892.4 |
| A302 | 1088.43 | 1088.4 |
| A303 | 1136.478 | 1136.55 |
| A304 | 1061.408 | 1061.55 |
| A305 | 1088.43 | 1088.55 |
| A306 | 1078.391 | 1078.55 |
| A307 | 1078.391 | 1078.55 |
| A308 | 1062.436 | 1062.65 |
| A309 | 1038.326 | 1038.5 |
| A310 | 1066.38 | 1066.4 |
| A311 | 1066.38 | 1066.45 |
| A312 | 1038.326 | 1038.55 |
| A313 | 921.178 | 921.35 |
| A314 | 1050.381 | 1050.5 |
| A315 | 852.071 | 852.25 |
| A316 | 1044.352 | 1044.55 |
| A317 | 1078.391 | 1078.55 |
| A318 | 1078.391 | 1078.6 |
| A319 | 1104.429 | 1104.6 |
| A320 | 1050.381 | 1050.5 |
| A321 | 1062.392 | 1062.35 |
| A322 | 1062.392 | 1062.4 |
| A323 | 1020.307 | 1020.4 |
| A324 | 892.092 | 892.35 |
| A325 | 880.081 | 880.3 |
| A326 | 851.058 | 851.4 |
| A327 | 851.058 | 851.4 |
| A328 | 879.068 | 879.35 |
| A329 | 1144.494 | 1144.45 |
| A330 | 864.082 | 864.4 |
| A331 | 1076.419 | 1076.55 |
| A332 | 1048.409 | 1048.55 |
| A333 | 1123.479 | 1123.45 |
| A334 | 1141.45 | 1141.6 |
| A335 | 1124.467 | 1124.4 |
| A336 | 1113.44 | 1113.45 |
| A337 | 1060.376 | 1060.5 |
| A338 | 1088.43 | 1088.5 |
| A339 | 1038.326 | 1038.35 |
| A340 | 1038.326 | 1038.35 |
| A341 | 866.054 | 866.4 |
| A342 | 867.057 | 867.55 |
| A343 | 1063.376 | 1063.65 |
| A344 | 1077.407 | 1077.6 |
| A345 | 1116.44 | 1116.7 |
| A346 | 1135.49 | 1135.65 |
| A347 | 1152.477 | 1152.7 |
| A348 | 1127.467 | 1127.6 |
| A349 | 1092.418 | 1092.45 |
| A350 | 1050.381 | 1050.6 |
| A351 | 1050.381 | 1050.55 |
| A352 | 1031.334 | 1031.45 |
| A353 | 1125.451 | 1125.65 |
| A354 | 854.043 | 854.35 |
| A355 | 975.245 | 975.75 |
| A356 | 1003.255 | 1003.75 |
| A357 | 1063.376 | 1063.7 |
| A358 | 1076.419 | 1076.65 |
| A359 | 1124.467 | 1124.55 |
| A360 | 1113.44 | 1113.6 |
| A361 | 1036.354 | 1036.6 |
| A362 | 1116.44 | 1116.6 |
| A363 | 1124.441 | 1124.65 |
| A364 | 1076.375 | 1076.6 |
| A365 | 1104.429 | 1104.7 |
| A366 | 1078.366 | 1078.55 |
| A367 | 992.257 | 992.6 |
| A368 | 1077.407 | 1077.75 |
| A369 | 1046.324 | 1046.6 |
| A370 | 1123.479 | 1123.65 |
| A371 | 1034.382 | 1034.65 |
| A372 | 1098.429 | 1098.65 |
| A373 | 1097.441 | 1097.65 |
| A374 | 1062.392 | 1062.6 |
| A375 | 1128.455 | 1128.65 |
| A376 | 1064.408 | 1064.59 |
| A377 | 954.204 | 954.6 |
| A378 | 954.204 | 954.6 |
| A379 | 1135.393 | 1135.65 |
| A380 | 1124.467 | 1124.65 |
| A381 | 1123.479 | 1123.65 |
| A382 | 1138.468 | 1138.5 |
| A383 | 1096.431 | 1096.55 |
| A384 | 1104.429 | 1104.7 |
| A385 | 1076.419 | 1076.65 |
| A386 | 1104.429 | 1104.65 |
| A387 | 1114.403 | 1114.6 |
| A388 | 992.253 | 992.6 |
| A389 | 1110.458 | 1110.5 |
| A390 | 987.281 | 987.55 |
| A391 | 1004.268 | 1004.5 |
| A392 | 1004.268 | 1004.65 |
| A393 | 980.242 | 980.4 |
| A394 | 1062.392 | 1062.65 |
| A395 | 1090.402 | 1090.6 |
| A396 | 1113.415 | 1113.55 |
| A397 | 1137.422 | 1137.65 |
| A398 | 1037.338 | 1038.2 |
| A399 | 1023.311 | 1024.2 |
| A400 | 1090.402 | 1090.45 |
| A401 | 1090.402 | 1090.5 |
| A402 | 1070.375 | 1070.55 |
| A403 | 1034.338 | 1034.65 |
| A404 | 1034.338 | 1034.65 |
| A405 | 1008.3 | 1008.55 |
| A406 | 1038.326 | 1038.55 |
| A407 | 1062.348 | 1062.5 |
| A408 | 1062.348 | 1062.5 |
| A409 | 1050.337 | 1050.55 |
| A410 | 1050.337 | 1050.55 |
| A411 | 926.194 | 926.5 |
| A412 | 926.194 | 926.55 |
| A413 | 952.232 | 952.55 |
| A414 | 1134.405 | 1134.7 |
| A415 | 1124.366 | 1124.75 |
| A416 | 1096.431 | 1096.55 |
| A417 | 1124.441 | 1124.6 |
| A418 | 1124.441 | 1124.65 |
| A419 | 892.092 | 892.5 |
| A420 | 1075.387 | 1075.5 |
| A421 | 1075.387 | 1075.55 |
| A422 | 1048.365 | 1048.65 |
| A423 | 1088.43 | 1088.65 |
| A424 | 1151.489 | 1151.75 |
| A425 | 1069.387 | 1069.6 |
| A426 | 1100.401 | 1100.6 |
| A427 | 1064.364 | 1064.65 |
| A428 | 1064.364 | 1064.6 |
| A429 | 1088.386 | 1088.65 |
| A430 | 1088.386 | 1088.6 |
| A431 | 1076.419 | 1076.6 |
| A432 | 864.082 | 864.5 |
| A433 | 1076.375 | 1076.55 |
| A434 | 1076.375 | 1076.7 |
| A435 | 1136.434 | 1136.65 |
| A436 | 1035.322 | 1035.45 |
| A437 | 1060.376 | 1060.6 |
| A438 | 1139.478 | 1139.6 |
| A439 | 1076.419 | 1076.65 |
| A440 | 880.081 | 880.5 |
| A441 | 864.082 | 864.45 |
| A442 | 864.082 | 864.45 |
| A443 | 992.257 | 992.55 |
| A444 | 952.232 | 952.45 |
| A445 | 1076.419 | 1076.6 |
| A446 | 1140.466 | 1140.6 |
| A447 | 1129.439 | 1129.6 |
| A448 | 921.178 | 921.55 |
| A449 | 1099.413 | 1099.65 |
| A450 | 980.242 | 980.6 |
| A451 | 1046.393 | 1046.7 |
| A452 | 1074.403 | 1074.75 |
| A453 | 1076.419 | 1076.65 |
| A454 | 1104.429 | 1104.75 |
| A455 | 1090.402 | 1090.65 |
| A456 | 1049.353 | 1049.45 |
| A457 | 1068.334 | 1068.65 |
| A458 | 1032.297 | 1032.6 |
| A459 | 1058.364 | 1058.6 |
| A460 | 1070.318 | 1070.6 |
| A461 | 1062.392 | 1062.55 |
| A462 | 852.071 | 852.5 |
| A463 | 1095.425 | 1095.65 |
| A464 | 1008.3 | 1008.6 |
| A465 | 1033.354 | 1033.6 |
| A466 | 1088.43 | 1088.7 |
| A467 | 1088.43 | 1088.7 |
| A468 | 1071.302 | 1071.55 |
| A469 | 1060.376 | 1060.6 |
| A470 | 1033.354 | 1033.55 |
| A471 | 1104.429 | 1104.65 |
| A472 | 1104.429 | 1104.65 |
| A473 | 1048.296 | 1049.6 |
| A474 | 1042.308 | 1042.6 |
| A475 | 1111.424 | 1111.65 |
| A476 | 1060.376 | 1060.6 |
| A477 | 1063.311 | 1063.7 |
| A478 | 1064.364 | 1064.6 |
| A479 | 1084.333 | 1084.6 |
| A480 | 1083.345 | 1083.6 |
| A481 | 1073.306 | 1073.55 |
| A482 | 1034.269 | 1034.6 |
| A483 | 1005.275 | 1005.55 |
| A484 | 1067.346 | 1067.7 |
| A485 | 1057.307 | 1057.55 |
| A486 | 1018.27 | 1018.55 |
| A487 | 1071.359 | 1071.65 |
| A488 | 1095.4 | 1095.7 |
| A489 | 1095.4 | 1095.75 |
| A490 | 1085.361 | 1085.75 |
| A491 | 1085.361 | 1085.7 |
| A492 | 1104.429 | 1104.65 |
| A493 | 1104.429 | 1104.65 |
| A494 | 1060.332 | 1060.65 |
| A495 | 1070.371 | 1070.75 |
| A496 | 1067.339 | 1067.6 |
| A497 | 1067.339 | 1067.65 |
| A498 | 861.013 | 861.45 |
| A499 | 1038.326 | 1038.6 |
| A500 | 1106.351 | 1106.65 |
| A501 | 1035.322 | 1035.55 |
| A502 | 1046.393 | 1046.4 |
| A503 | 1059.348 | 1059.6 |
| A504 | 1112.456 | 1112.7 |
| A505 | 1091.39 | 1091.5 |
| A506 | 1127.467 | 1127.65 |
| A507 | 1111.468 | 1111.65 |
| A508 | 1101.429 | 1101.65 |
| A509 | 1038.326 | 1038.6 |
| A510 | 1076.375 | 1076.6 |
| A511 | 1096.413 | 1096.6 |
| A512 | 1020.267 | 1020.4 |
| A513 | 1036.354 | 1036.75 |
| A514 | 1067.339 | 1067.65 |
| A515 | 1067.339 | 1067.65 |
| A516 | 979.189 | 979.45 |
| A517 | 1032.278 | 1032.5 |
| A518 | 1044.28 | 1044.55 |
| A519 | 1038.326 | 1038.55 |
| A520 | 1038.326 | 1038.55 |
| A521 | 1008.256 | 1009 |
| A522 | 1115.412 | 1115.5 |
| A523 | 1078.366 | 1078.5 |
| A524 | 1078.366 | 1078.55 |
| A525 | 1112.43 | 1112.4 |
| A526 | 1071.377 | 1071.3 |
| A527 | 1107.411 | 1107.6 |
| A528 | 1107.411 | 1107.65 |
| A529 | 1097.372 | 1097.65 |
| A530 | 1064.364 | 1064.6 |
| A531 | 1044.324 | 1044.6 |
| A532 | 937.177 | 937.5 |
| A533 | 1096.413 | 1096.55 |
| A534 | 1065.377 | 1065.45 |
| A535 | 1065.377 | 1065.5 |
| A536 | 1064.383 | 1064.5 |
| A537 | 1107.414 | 1107.45 |
| A538 | 1132.468 | 1132.4 |
| A539 | 979.189 | 979.4 |
| A540 | 1112.456 | 1112.65 |
| A541 | 1097.372 | 1097.65 |
| A542 | 1127.467 | 1127.55 |
| A543 | 1111.468 | 1111.65 |
| A544 | 1117.428 | 1117.4 |
| A545 | 1117.428 | 1117.6 |
| A546 | 1101.429 | 1101.6 |
| A547 | 1135.393 | 1135.4 |
| A548 | 1135.393 | 1135.4 |
| A549 | 1060.376 | 1060.55 |
| A550 | 1060.376 | 1060.5 |
| A551 | 1020.311 | 1020.45 |
| A552 | 1101.385 | 1101.55 |
| A553 | 908.179 | 908.3 |
| A554 | 1076.35 | 1076.4 |
| A555 | 1093.381 | 1093.4 |
| A556 | 1020.311 | 1020.55 |
| A557 | 1096.413 | 1096.6 |
| A558 | 1047.308 | 1047.7 |
| A559 | 1055.328 | 1055.6 |
| A560 | 1055.328 | 1055.6 |
| A561 | 1128.455 | 1128.6 |
| A562 | 1073.322 | 1073.35 |
| A563 | 1073.322 | 1073.35 |
| A564 | 967.178 | 967.4 |
| A565 | 967.178 | 967.45 |
| A566 | 1047.337 | 1047.7 |
| A567 | 1057.323 | 1057.4 |
| A568 | 1057.323 | 1057.35 |
| A569 | 937.177 | 937.35 |
| A570 | 1062.352 | 1062.65 |
| A571 | 1047.308 | 1047.6 |
| A572 | 1043.28 | 1043.65 |
| A573 | 1043.28 | 1043.65 |
| A574 | 1086.414 | 1086.55 |
| A575 | 1008.3 | 1008.4 |
| A576 | 969.263 | 969.5 |
| A577 | 1086.317 | 1086.45 |
| A578 | 1086.418 | 1086.45 |
| A579 | 1061.364 | 1061.4 |
| A580 | 1008.281 | 1008.35 |
| A581 | 1087.402 | 1087.4 |
| A582 | 1128.455 | 1128.75 |
| A583 | 1063.38 | 1063.55 |
| A584 | 1086.414 | 1086.55 |
| A585 | 1076.375 | 1076.45 |
| A586 | 1076.375 | 1076.45 |
| A587 | 1096.344 | 1096.4 |
| A588 | 1059.291 | 1059.65 |
| A589 | 1059.291 | 1059.6 |
| A590 | 1023.311 | 1023.6 |
| A591 | 1064.364 | 1064.45 |
| A592 | 1112.412 | 1112.55 |
| A593 | 1086.418 | 1086.5 |
| A594 | 1001.224 | 1001.4 |
| A595 | 1080.345 | 1080.7 |
| A596 | 1089.374 | 1089.5 |
| A597 | 1048.321 | 1048.55 |
| A598 | 1048.365 | 1048.4 |
| A599 | 1089.374 | 1089.35 |
| A600 | 1048.321 | 1048.4 |
| A601 | 1071.359 | 1071.45 |
| A602 | 1117.403 | 1117.45 |
| A603 | 1117.403 | 1117.45 |
| A604 | 1117.403 | 1117.65 |
| A605 | 1117.403 | 1117.6 |
| A606 | 1089.374 | 1089.45 |
| A607 | 1111.424 | 1111.45 |
| A608 | 1087.402 | 1087.45 |
| A609 | 1058.263 | 1058.35 |
| A610 | 1047.337 | 1047.65 |
| A611 | 1047.337 | 1047.55 |
| A612 | 1064.364 | 1064.55 |
| A613 | 924.178 | 924.45 |
| A614 | 1048.365 | 1048.75 |
| A615 | 924.178 | 924.45 |
| A616 | 1056.335 | 1056.55 |
| A617 | 1062.348 | 1062.55 |
| A618 | 1036.31 | 1036.45 |
| A619 | 1101.385 | 1101.45 |
| A620 | 1112.412 | 1112.45 |
| A621 | 1051.344 | 1051.65 |
| A622 | 995.257 | 995.4 |
| A623 | 1107.364 | 1107.55 |
| A624 | 1107.364 | 1107.55 |
| A625 | 1079.379 | 1079.4 |
| A626 | 1059.348 | 1059.4 |
| A627 | 1114.384 | 1114.5 |
| A628 | 1101.385 | 1101.35 |
| A629 | 978.205 | 978.4 |
| A630 | 1036.31 | 1036.55 |
| A631 | 1044.28 | 1044.7 |
| A632 | 1084.402 | 1084.75 |
| A633 | 1063.336 | 1063.35 |
| A634 | 1063.336 | 1063.35 |
| A635 | 1048.365 | 1048.45 |
| A636 | 1076.375 | 1076.75 |
| A637 | 1076.375 | 1076.75 |
| A638 | 1073.375 | 1073.4 |
| A639 | 1099.413 | 1099.6 |
| A640 | 1046.393 | 1046.45 |
| A641 | 1032.297 | 1032.6 |
| A642 | 1031.237 | 1031.45 |
| A643 | 1087.345 | 1087.55 |
| A644 | 1076.419 | 1076.4 |
| A645 | 1076.419 | 1076.45 |
| A646 | 877.081 | 877.3 |
| A647 | 978.205 | 978.35 |
| A648 | 969.263 | 971.1 |
| A649 | 1032.366 | 1032.75 |
| A650 | 1052.328 | 1052.5 |
| A651 | 1064.364 | 1064.75 |
| A652 | 1026.29 | 1026.5 |
| A653 | 1038.326 | 1038.15 |
| A654 | 1091.365 | 1091.51 |
| A655 | 1103.401 | 1103.15 |
| A656 | 1054.275 | 1054.3 |
| A657 | 1018.295 | 1018.65 |
| A658 | 1059.348 | 1059.4 |
| A659 | 1057.275 | 1057.35 |
| A660 | 1021.295 | 1021.55 |
| A661 | 1062.348 | 1062.15 |
| A662 | 1056.247 | 1056.6 |
| A663 | 1059.348 | 1059.55 |
| A664 | 1033.31 | 1033.45 |
| A665 | 1071.277 | 1071.75 |
| A666 | 1035.297 | 1035.6 |
| A667 | 1092.418 | 1092.7 |
| A668 | 1092.418 | 1092.7 |
| A669 | 990.163 | 990.5 |
| A670 | 1096.387 | 1096.35 |
| A671 | 971.141 | 971.35 |
| A672 | 960.215 | 960.35 |
| A673 | 976.214 | 976.35 |
| A674 | 1059.348 | 1059.55 |
| A675 | 1098.385 | 1098.7 |
| A676 | 960.114 | 960.4 |
| A677 | 970.153 | 970.4 |
| A678 | 1020.267 | 1020.5 |
| A679 | 1061.32 | 1061.55 |
| A680 | 1033.31 | 1033.5 |
| A681 | 1098.385 | 1098.75 |
| A682 | 1051.365 | 526.6 |
| A683 | 1051.365 | 1051.45 |
| A684 | 841.048 | 841.45 |
| A685 | 1043.267 | 1043.65 |
| A686 | 1043.267 | 1043.65 |
| A687 | 969.263 | 970 |
| A688 | 955.236 | 955.5 |
| A689 | 910.151 | 910.3 |
| A690 | 896.168 | 896.35 |
| A691 | 912.167 | 912.4 |
| A692 | 1059.266 | 1059.6 |
| A693 | 1059.266 | 1059.55 |
| A694 | 1100.3 | 1100.55 |
| A695 | 1084.301 | 1084.6 |
| A696 | 1084.301 | 1084.55 |
| A697 | 929.198 | 929.9 |
| A698 | 954.208 | 954.9 |
| A699 | 954.208 | 954.9 |
| A700 | 893.08 | 893.4 |
| A701 | 854.043 | 854.4 |
| A702 | 1080.244 | 1080.65 |
| A703 | 1067.346 | 1067.55 |
| A704 | 1015.213 | 1015.5 |
| A705 | 1100.3 | 1100.55 |
| A706 | 1084.232 | 1084.5 |
| A707 | 1094.271 | 1094.45 |
| A708 | 1068.233 | 1068.65 |
| A709 | 1078.272 | 1078.7 |
| A710 | 1098.275 | 1098.6 |
| A711 | 1057.307 | 1057.5 |
| A712 | 1004.287 | 1004.55 |
| A713 | 837.06 | 837.3 |
| A714 | 955.236 | 955.9 |
| A715 | 955.236 | 956 |
| A716 | 1031.338 | 1031.35 |
| A717 | 1080.285 | 1080.4 |
| A718 | 1034.338 | 1034.4 |
| A719 | 1008.3 | 1008.4 |
| A720 | 1021.343 | 1021.4 |
| A721 | 1100.369 | 1100.6 |
| A722 | 1100.369 | 1100.6 |
| A723 | 1008.3 | 1008.55 |
| A724 | 1096.312 | 1096.35 |
| A725 | 995.301 | 995.35 |
| A726 | 1049.353 | 1049.45 |
| A727 | 799.007 | 800 |
| A728 | 1060.332 | 1060.5 |
| A729 | 1083.414 | 1083.4 |
| A730 | 1073.375 | 1073.45 |
| A731 | 1083.345 | 1083.45 |
| A732 | 1101.385 | 1101.4 |
| A733 | 1073.306 | 1073.4 |
| A734 | 1020.355 | 1020.5 |
| A735 | 1013.291 | 1013.5 |
| A736 | 1013.291 | 1013.55 |
| A737 | 1013.291 | 1013.55 |
| A738 | 1013.291 | 1013.5 |
| A739 | 1048.34 | 1048.45 |
| A740 | 899.168 | 899.5 |
| A741 | 899.168 | 899.5 |
| A742 | 1099.413 | 1099.45 |
| A743 | 1024.299 | 1024.55 |
| A744 | 965.187 | 965.4 |
| A745 | 1032.253 | 1032.6 |
| A746 | 1084.301 | 1084.45 |
| A747 | 1095.328 | 1095.6 |
| A748 | 1095.328 | 1095.6 |
| A749 | 1008.344 | 1008.4 |
| A750 | 1050.337 | 1050.4 |
| A751 | 913.155 | 913.5 |
| A752 | 1024.299 | 1024.45 |
| A753 | 975.226 | 975.4 |
| A754 | 1023.286 | 1023.5 |
| A755 | 1023.286 | 1023.55 |
| A756 | 1110.339 | 1110.45 |
| A757 | 1074.359 | 1074.45 |
| A758 | 1042.292 | 1042.5 |
| A759 | 1007.287 | 1007.55 |
| A760 | 979.233 | 979.55 |
| A761 | 1084.301 | 1084.45 |
| A762 | 1074.306 | 1074.7 |
| A763 | 948.191 | 948.45 |
| A764 | 1007.312 | 1007.55 |
| A765 | 1045.308 | 1045.7 |
| A766 | 1036.354 | 1036.55 |
| A767 | 1047.337 | 1047.4 |
| A768 | 1024.299 | 1024.45 |
| A769 | 1006.284 | 1006.4 |
| A770 | 1009.284 | 1009.45 |
| A771 | 1058.291 | 1058.6 |
| A772 | 1048.252 | 1048.55 |
| A773 | 1100.3 | 1100.45 |
| A774 | 1064.32 | 1064.5 |
| A775 | 1094.34 | 1094.5 |
| A776 | 1058.36 | 1058.55 |
| A777 | 1048.321 | 1048.65 |
| A778 | 1100.369 | 1100.4 |
| A779 | 1084.301 | 1084.35 |
| A780 | 1090.305 | 1090.6 |
| A781 | 1043.292 | 1043.45 |
| A782 | 995.232 | 995.5 |
| A783 | 1095.328 | 1095.5 |
| A784 | 1095.328 | 1095.5 |
| A785 | 1105.442 | 1105.35 |
| A786 | 1067.368 | 1068.1 |
| A787 | 852.977 | 853.7 |
| A788 | 1042.314 | 1042.6 |
| A789 | 1059.335 | 1059.55 |
| A790 | 1026.315 | 1026.5 |
| A791 | 1038.326 | 1038.55 |
| A792 | 1023.355 | 1023.55 |
| A793 | 1048.268 | 1048.45 |
| A794 | 1061.307 | 1061.5 |
| A795 | 1025.327 | 1025.55 |
| A796 | 1089.374 | 1089.65 |
| A797 | 1089.374 | 1089.65 |
| A798 | 1100.401 | 1100.45 |
| A799 | 1100.401 | 1100.5 |
| A800 | 1038.37 | 1038.55 |
| A801 | 1038.37 | 1038.55 |
| A802 | 1012.288 | 1012.55 |
| A803 | 1039.354 | 1039.55 |
| A804 | 1039.354 | 1039.5 |
| A805 | 995.301 | 995.5 |
| A806 | 1032.269 | 1032.5 |
| A807 | 1009.328 | 1009.75 |
| A808 | 1019.27 | 1019.5 |
| A809 | 1075.334 | 1075.8 |
| A810 | 1053.341 | 1053.5 |
| A811 | 1006.231 | 1006.5 |
| A812 | 983.29 | 983.4 |
| A813 | 1011.3 | 1011.75 |
| A814 | 1037.342 | 1037.75 |
| A815 | 1100.401 | 1100.7 |
| A816 | 1100.401 | 1100.7 |
| A817 | 1012.288 | 1013 |
| A818 | 1048.268 | 1048.45 |
| A819 | 1090.406 | 546.1 |
| A820 | 1047.28 | 1047.2 |
| A821 | 991.273 | 992.1 |
| A822 | 1148.486 | 1148.8 |
| A823 | 1062.295 | 1062.55 |
| A824 | 1032.269 | 1032.7 |
| A825 | 1023.355 | 512.5 |
| A826 | 983.29 | 983.7 |
| A827 | 1093.45 | 1093.4 |
| A828 | 1079.423 | 540.6 |
| A829 | 1079.423 | 1079.4 |
| A830 | 1101.376 | 1101.8 |
| A831 | 968.279 | 968.4 |
| A832 | 1073.371 | 1073.55 |
| A833 | 1073.371 | 1073.55 |
| A834 | 1033.297 | 1033.7 |
| A835 | 1033.297 | 1033.7 |
| A836 | 1023.355 | 512.5 |
| A837 | 982.302 | 982.4 |
| A838 | 996.314 | 996.5 |
| A839 | 982.302 | 982.5 |
| A840 | 1032.322 | 1032.35 |
| A841 | 1018.282 | 1018.9 |
| A842 | 1018.282 | 1018.9 |
| A843 | 1047.28 | 1047.8 |
| A844 | 1047.28 | 1047.8 |
| A845 | 1018.295 | 509.9 |
| A846 | 980.246 | 980.5 |
| A847 | 994.22 | 994.4 |
| A848 | 1039.358 | 1039.4 |
| A849 | 993.289 | 993.55 |
| A850 | 1073.371 | 1073.8 |
| A851 | 1073.371 | 1073.8 |
| A852 | 970.251 | 970.8 |
| A853 | 990.285 | 990.4 |
| A854 | 996.289 | 997 |
| A855 | 969.263 | 970 |
| A856 | 1010.316 | 1010.4 |
| A857 | 1031.281 | 1031.7 |
| A858 | 1032.309 | 1032.3 |
| A859 | 1035.269 | 1035.7 |
| A860 | 1011.3 | 1011.7 |
| A861 | 952.207 | 952.5 |
| A862 | 996.329 | 997 |
| A863 | 996.329 | 996.9 |
| A864 | 1047.28 | 1047.6 |
| A865 | 928.214 | 928.4 |
| A866 | 958.24 | 958.4 |
| A867 | 976.258 | 976.9 |
| A868 | 829.077 | 829.7 |

Note:
values may differ slightly from values found elsewhere in this application due to different measurements and rounding.

TABLE 6
Exemplary Compounds Prepared by
Methods of the Present Invention

| | Molecular | |
|---|---|---|
| | weight | MS |
| Ex# | (g/mol) | Found |
| A869 | 932.160 | 932.7 |
| A870 | 996.206 | 996.7 |
| A871 | 937.200 | 937.7 |
| A872 | 996.272 | 996.7 |
| A873 | 1001.246 | 1001.7 |
| A874 | 934.199 | 934.7 |
| A875 | 998.245 | 998.6 |
| A876 | 911.184 | 911.7 |
| A877 | 948.226 | 948.6 |
| A878 | 918.157 | 918.4 |
| A879 | 1012.271 | 1012.6 |
| A880 | 1106.429 | 1106.5 |
| A881 | 928.172 | 928.6 |
| A882 | 928.172 | 928.7 |
| A883 | 930.188 | 930.6 |
| A884 | 944.237 | 944.6 |
| A885 | 944.237 | 944.6 |
| A886 | 932.227 | 932.6 |
| A887 | 946.253 | 946.7 |
| A888 | 1010.299 | 1010.6 |
| A889 | 1010.299 | 1010.6 |
| A890 | 813.986 | 814.5 |
| A891 | 878.032 | 878.6 |
| A892 | 894.097 | 894.4 |
| A893 | 922.150 | 922.4 |
| A894 | 921.176 | 921.6 |
| A895 | 968.219 | 968.5 |
| A896 | 985.221 | 985.5 |
| A897 | 1032.264 | 1032.6 |
| A898 | 1076.337 | 1076.8 |
| A899 | 1026.318 | 1026.6 |
| A900 | 930.144 | 930.5 |
| A901 | 930.188 | 930.6 |
| A902 | 955.154 | 955.5 |
| A903 | 932.227 | 932.5 |
| A904 | 965.253 | 965.4 |
| A905 | 895.163 | 895.5 |
| A906 | 905.158 | 905.5 |
| A907 | 888.131 | 888.3 |
| A908 | 865.094 | 865.5 |
| A909 | 891.056 | 891.5 |
| A910 | 996.272 | 996.5 |
| A911 | 959.209 | 959.5 |
| A912 | 969.204 | 969.5 |
| A913 | 929.140 | 929.5 |
| A914 | 955.101 | 955.5 |
| A915 | 951.227 | 951.4 |
| A916 | 1015.272 | 1015.4 |
| A917 | 813.986 | 814.7 |
| A918 | 878.032 | 878.6 |
| A919 | 830.052 | 830.5 |
| A920 | 830.052 | 830.3 |
| A921 | 894.097 | 894.5 |
| A922 | 828.013 | 828.4 |
| A923 | 908.124 | 908.4 |
| A924 | 908.124 | 908.6 |
| A925 | 828.013 | 828.6 |
| A926 | 828.013 | 828.5 |
| A927 | 892.058 | 892.6 |
| A928 | 892.058 | 892.6 |
| A929 | 844.078 | 844.5 |
| A930 | 844.078 | 844.4 |
| A931 | 908.124 | 908.4 |
| A932 | 908.124 | 908.4 |
| A933 | 858.105 | 858.6 |
| A934 | 921.176 | 921.4 |
| A935 | 985.221 | 985.4 |
| A936 | 946.210 | 946.3 |
| A937 | 1010.256 | 1010.4 |
| A938 | 1090.363 | 1090.6 |
| A939 | 888.108 | 888.6 |
| A940 | 946.210 | 946.6 |
| A941 | 946.210 | 946.6 |
| A942 | 946.253 | 946.7 |
| A943 | 946.253 | 946.4 |
| A944 | 971.220 | 971.7 |
| A945 | 888.131 | 888.6 |
| A946 | 837.084 | 837.5 |
| A947 | 1029.299 | 1029.5 |
| A948 | 952.176 | 952.5 |
| A949 | 952.176 | 952.5 |
| A950 | 901.130 | 901.6 |
| A951 | 909.190 | 909.5 |
| A952 | 905.082 | 905.5 |
| A953 | 851.111 | 851.6 |
| A954 | 973.235 | 973.5 |
| A955 | 966.203 | 966.4 |
| A956 | 969.128 | 969.4 |
| A957 | 915.156 | 915.5 |
| A958 | 892.058 | 892.5 |
| A959 | 892.058 | 892.5 |
| A960 | 844.078 | 844.6 |
| A961 | 844.078 | 844.4 |
| A962 | 842.039 | 842.4 |
| A963 | 906.085 | 906.4 |
| A964 | 906.085 | 906.5 |
| A965 | 858.105 | 858.4 |
| A966 | 922.150 | 922.4 |
| A967 | 891.084 | 891.6 |
| A968 | 982.245 | 982.6 |
| A969 | 982.179 | 982.6 |
| A970 | 1046.291 | 1046.6 |
| A971 | 1028.357 | 1028.5 |
| A972 | 1092.402 | 1092.5 |
| A973 | 979.280 | 979.5 |
| A974 | 919.185 | 919.6 |
| A975 | 879.121 | 879.6 |
| A976 | 1043.325 | 1043.5 |
| A977 | 983.230 | 983.6 |
| A978 | 966.203 | 966.5 |
| A979 | 943.166 | 943.6 |
| A980 | 828.013 | 828.3 |
| A981 | 955.129 | 955.5 |
| A982 | 1012.291 | 1012.6 |
| A983 | 902.158 | 902.6 |
| A984 | 879.016 | 879.6 |
| A985 | 879.016 | 879.4 |
| A986 | 814.971 | 815.5 |
| A987 | 987.305 | 987.7 |
| A988 | 895.082 | 895.4 |
| A989 | 895.082 | 895.4 |
| A990 | 831.037 | 831.4 |
| A991 | 1045.318 | 1045.6 |
| A992 | 1061.384 | 1061.7 |
| A993 | 932.183 | 932.6 |
| A994 | 996.229 | 996.5 |
| A995 | 904.173 | 904.5 |
| A996 | 895.082 | 895.4 |
| A997 | 895.082 | 895.4 |
| A998 | 879.016 | 879.3 |
| A999 | 814.971 | 815.3 |
| A1000 | 879.016 | 879.6 |
| A1001 | 904.130 | 904.6 |
| A1002 | 891.089 | 891.5 |
| A1003 | 968.237 | 968.6 |
| A1004 | 968.176 | 968.6 |
| A1005 | 1032.283 | 1032.4 |
| A1006 | 1038.309 | 1038.5 |
| A1007 | 1045.318 | 1045.5 |
| A1008 | 1081.310 | 1081.5 |
| A1009 | 831.037 | 831.3 |
| A1010 | 831.037 | 831.4 |
| A1011 | 878.133 | 878.6 |
| A1012 | 884.113 | 884.4 |
| A1013 | 948.158 | 948.5 |
| A1014 | 1061.384 | 1061.6 |
| A1015 | 1097.376 | 1097.6 |
| A1016 | 954.192 | 954.6 |
| A1017 | 890.147 | 890.5 |
| A1018 | 952.153 | 952.6 |
| A1019 | 888.108 | 888.5 |
| A1020 | 914.145 | 914.5 |
| A1021 | 978.191 | 978.6 |
| A1022 | 942.178 | 942.5 |
| A1023 | 1036.270 | 1036.7 |
| A1024 | 1147.430 | 1148.8 |
| A1025 | 900.119 | 900.5 |
| A1026 | 964.164 | 964.5 |
| A1027 | 934.199 | 934.6 |
| A1028 | 921.201 | 921.3 |
| A1029 | 998.245 | 998.6 |
| A1030 | 985.246 | 985.3 |
| A1031 | 903.119 | 903.4 |
| A1032 | 938.127 | 938.6 |
| A1033 | 874.081 | 874.7 |
| A1034 | 902.134 | 902.6 |
| A1035 | 980.163 | 980.5 |
| A1036 | 916.118 | 916.6 |
| A1037 | 895.139 | 895.5 |
| A1038 | 907.174 | 907.5 |
| A1039 | 907.154 | 907.5 |
| A1040 | 907.154 | 907.4 |
| A1041 | 902.133 | 902.4 |
| A1042 | 982.264 | 982.6 |
| A1043 | 988.290 | 988.7 |
| A1044 | 959.184 | 959.5 |
| A1045 | 985.203 | 985.5 |
| A1046 | 971.220 | 971.3 |
| A1047 | 930.168 | 930.4 |
| A1048 | 971.200 | 971.6 |
| A1049 | 966.178 | 966.5 |
| A1050 | 1052.335 | 1052.7 |
| A1051 | 982.245 | 982.6 |
| A1052 | 960.237 | 960.7 |
| A1053 | 918.200 | 918.7 |
| A1054 | 907.149 | 907.6 |
| A1055 | 908.159 | 908.4 |
| A1056 | 894.132 | 894.5 |
| A1057 | 866.122 | 866.5 |
| A1058 | 971.195 | 971.5 |
| A1059 | 972.204 | 972.4 |
| A1060 | 970.232 | 970.4 |
| A1061 | 958.178 | 958.5 |
| A1062 | 971.200 | 971.4 |
| A1063 | 919.185 | 919.5 |
| A1064 | 919.185 | 919.5 |
| A1065 | 1065.377 | 1065.5 |
| A1066 | 904.173 | 904.5 |
| A1067 | 901.126 | 901.5 |
| A1068 | 901.126 | 901.6 |
| A1069 | 885.061 | 885.6 |
| A1070 | 885.061 | 885.7 |
| A1071 | 965.172 | 965.6 |
| A1072 | 965.172 | 965.6 |
| A1073 | 949.106 | 949.6 |
| A1074 | 949.106 | 949.6 |
| A1075 | 1001.332 | 1001.8 |
| A1076 | 1010.256 | 1010.5 |
| A1077 | 968.219 | 968.4 |
| A1078 | 1046.309 | 1046.5 |
| A1079 | 946.210 | 946.5 |
| A1080 | 904.173 | 904.5 |
| A1081 | 1024.282 | 1024.6 |
| A1082 | 1035.285 | 1035.8 |
| A1083 | 1160.472 | 1161.2 |
| A1084 | 955.177 | 955.6 |
| A1085 | 1133.400 | 1134.8 |
| A1086 | 966.180 | 966.6 |
| A1087 | 1167.484 | 1167.7 |
| A1088 | 1117.400 | 1116.7 |
| A1089 | 1000.233 | 1000.5 |
| A1090 | 952.153 | 952.5 |
| A1091 | 888.108 | 888.5 |
| A1092 | 1181.489 | 1183.6 |
| A1093 | 1168.447 | 1169.4 |
| A1094 | 1238.583 | 1238.6 |
| A1095 | 1238.583 | 1239.7 |
| A1096 | 1221.531 | 1221.5 |
| A1097 | 1154.442 | 1154.7 |
| A1098 | 1108.351 | 1108.8 |
| A1099 | 968.219 | 968.5 |
| A1100 | 904.173 | 904.7 |
| A1101 | 1002.212 | 1002.5 |
| A1102 | 938.167 | 938.7 |
| A1103 | 968.219 | 968.6 |
| A1104 | 940.143 | 940.5 |
| A1105 | 876.097 | 876.4 |
| A1106 | 1022.257 | 1022.7 |
| A1107 | 1022.257 | 1022.7 |
| A1108 | 1086.302 | 1086.7 |
| A1109 | 1086.302 | 1086.7 |
| A1110 | 956.205 | 956.5 |
| A1111 | 892.159 | 892.5 |
| A1112 | 996.229 | 996.4 |
| A1113 | 932.183 | 932.5 |
| A1114 | 898.073 | 898.6 |
| A1115 | 962.118 | 962.6 |
| A1116 | 962.118 | 962.6 |
| A1117 | 1112.387 | 1113.5 |
| A1118 | 968.153 | 968.5 |
| A1119 | 904.107 | 904.5 |
| A1120 | 956.208 | 956.5 |
| A1121 | 892.163 | 892.5 |
| A1122 | 1063.356 | 1064.2 |
| A1123 | 1063.356 | 1064.2 |
| A1124 | 1124.416 | 1125.1 |
| A1125 | 1018.278 | 1018.4 |
| A1126 | 954.232 | 954.5 |
| A1127 | 939.111 | 939.4 |
| A1128 | 1027.260 | 1027.6 |
| A1129 | 1140.440 | 1141.1 |
| A1130 | 967.231 | 967.4 |
| A1131 | 984.218 | 984.6 |
| A1132 | 922.189 | 922.5 |
| A1133 | 1079.356 | 1080.2 |
| A1134 | 1079.356 | 1080.0 |
| A1135 | 1079.356 | 1080.0 |
| A1136 | 1012.295 | 1012.6 |
| A1137 | 1106.456 | 1106.5 |
| A1138 | 944.26 | 944.55 |
| A1139 | 894.119 | 894.4 |
| A1140 | 884.095 | 884.5 |
| A1141 | 858.057 | 858.55 |
| A1142 | 886.136 | 886.4 |
| A1143 | 860.073 | 860.45 |
| A1144 | 1076.361 | 1076.75 |
| A1145 | 888.152 | 888.3 |
| A1146 | 865.114 | 865.45 |
| A1147 | 891.074 | 891.5 |
| A1148 | 996.296 | 996.45 |
| A1149 | 959.231 | 959.5 |
| A1150 | 969.226 | 969.5 |
| A1151 | 929.161 | 929.45 |
| A1152 | 955.121 | 955.5 |
| A1153 | 908.146 | 908.4 |
| A1154 | 888.152 | 888.4 |
| A1155 | 900.163 | 900.45 |
| A1156 | 900.163 | 900.45 |
| A1157 | 1090.388 | 1090.6 |
| A1158 | 946.276 | 946.35 |
| A1159 | 971.242 | 971.65 |
| A1160 | 888.152 | 888.55 |
| A1161 | 952.199 | 952.5 |
| A1162 | 952.199 | 952.5 |
| A1163 | 901.151 | 901.55 |
| A1164 | 973.258 | 973.45 |
| A1165 | 966.226 | 966.35 |
| A1166 | 969.148 | 969.4 |
| A1167 | 915.178 | 915.5 |
| A1168 | 888.152 | 888.55 |
| A1169 | 1028.382 | 1028.5 |
| A1170 | 1092.429 | 1092.45 |
| A1171 | 983.253 | 983.55 |
| A1172 | 966.226 | 966.45 |
| A1173 | 943.188 | 943.55 |
| A1174 | 881.047 | 881.4 |
| A1175 | 1012.314 | 1012.6 |
| A1176 | 902.179 | 902.55 |
| A1177 | 902.179 | 902.45 |
| A1178 | 874.125 | 874.45 |
| A1179 | 874.125 | 874.4 |
| A1180 | 872.084 | 872.4 |
| A1181 | 1097.401 | 1097.55 |
| A1182 | 954.215 | 954.6 |
| A1183 | 890.168 | 890.45 |
| A1184 | 952.174 | 952.55 |
| A1185 | 938.147 | 938.6 |
| A1186 | 982.269 | 982.6 |
| A1187 | 886.111 | 886.4 |
| A1188 | 859.085 | 859.45 |
| A1189 | 924.12 | 924.45 |
| A1190 | 968.242 | 968.35 |
| A1191 | 904.195 | 904.45 |
| A1192 | 1133.424 | 1134.75 |
| A1193 | 968.242 | 968.5 |
| A1194 | 860.073 | 860.55 |
| A1195 | 886.111 | 886.5 |
| A1196 | 902.109 | 902.3 |
| A1197 | 1080.363 | 1080.6 |
| A1198 | 1022.283 | 1022.55 |
| A1199 | 1080.363 | 1080.35 |
| A1200 | 1080.363 | 1080.5 |

Note:
values may differ slightly from values found elsewhere in this application due to different measurements and rounding.

### Biological Assays

Disruption of B-Raf Ras-Binding Domain (BRAF^{RBD}) Interaction with K-Ras by Compounds of the Invention (Also Called a FRET Assay or an MOA Assay)

The purpose of this biochemical assay is to measure the ability of test compounds to facilitate ternary complex formation between a nucleotide-loaded K-Ras isoform and cyclophilin A; the resulting ternary complex disrupts binding to a BRAF^{RBD} construct, inhibiting K-Ras signaling through a RAF effector. Data is reported as IC50 values. Other Ras variants may be used.

In assay buffer containing 25 mM HEPES pH 7.3, 0.002% Tween20, 0.1% BSA, 100 mM NaCl and 5 mM MgCl₂, tagless cyclophilin A, His6-K-Ras-GMPPNP, and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a K-Ras: RAF complex are identified as those eliciting a decrease in the TR-FRET ratio relative to DMSO control wells.

Each of Examples A1-A868 and A876-A1196 exhibited an IC50 of less than 1 μM in with respect to K-RasG12V in this assay. Each of Examples A876-A1196 exhibited an IC50 of less than 1 μM in with respect to K-RasG12A in this assay.

Potency Assay: pERK

The purpose of this assay is to measure the ability of test compounds to inhibit K-Ras in cells. Activated K-Ras induces increased phosphorylation of ERK at Threonine 202 and Tyrosine 204 (PERK). This procedure measures a decrease in cellular pERK in response to test compounds. The procedure described below in Capan-1 cells is applicable to K-RasG12V.

Note: This protocol may be executed substituting other cell lines to characterize inhibitors of other RAS variants, including, for example, NCI-H₃₅₈ (K-Ras G12C), AsPC-1 (K-Ras G12D), Capan-1 (K-Ras G12V), or NCI-H₁₃₅₅ (K-Ras G13C).

Cells are grown and maintained using media and procedures recommended by the ATCC. On the day prior to compound addition, cells are plated in 384-well cell culture plates (40 μl/well) and grown overnight in a 37° C., 5% CO₂ incubator. Test compounds are prepared in 10, 3-fold dilutions in DMSO, with a high concentration of 10 mM. On the day of assay, 40 nL of test compound is added to each well of cell culture plate using an Echo550 liquid handler (LabCyte®). Concentrations of test compound are tested in duplicate. After compound addition, cells are incubated 4 hours at 37° C., 5% CO₂. Following incubation, culture medium is removed, and cells are washed once with phosphate buffered saline.

In some experiments, cellular pERK level is determined using the AlphaLISA SureFire Ultra p-ERK1/2 Assay Kit (PerkinElmer). Cells are lysed in 25 μL lysis buffer, with shaking at 600 RPM at room temperature. Lysate (10 μL) is transferred to a 384-well Opti-plate (PerkinElmer) and 5 μL acceptor mix is added. After a 2-hour incubation in the dark, 5 μL donor mix is added, plate is sealed, and incubated 2 hours at room temperature. Signal is read on an Envision plate reader (PerkinElmer) using standard AlphaLISA settings. Analysis of raw data is carried out in Excel (Microsoft) and Prism (GraphPad). Signal is plotted vs. the decadal logarithm of compound concentration, and IC₅₀ is determined by fitting a 4-parameter sigmoidal concentration response model.

In other experiments, cellular pERK is determined by In-Cell Western. Following compound treatment, cells are washed twice with 200 μL tris buffered saline (TBS) and fixed for 15 minutes with 150 μL 4% paraformaldehyde in TBS. Fixed cells are washed 4 times for 5 minutes with TBS containing 0.1% Triton X-100 (TBST) and then blocked with 100 μL Odyssey blocking buffer (LI-COR) for 60 minutes at room temperature. Primary antibody (PERK, CST-4370, Cell Signaling Technology) is diluted 1:200 in blocking buffer, and 50 μL is added to each well and incubated overnight at 4° C. Cells are washed 4 times for 5 minutes with TBST. Secondary antibody (IR-800CW rabbit, LI-COR, diluted 1:800) and DNA stain DRAQ5 (LI-COR, diluted 1:2000) are added and incubated 1-2 hours at room temperature. Cells are washed 4 times for 5 minutes with TBST. Plates are scanned on a Li—COR Odyssey CLx Imager. Analysis of raw data is carried out in Excel (Microsoft) and Prism (GraphPad). Signal is plotted vs. the decadal logarithm of compound concentration, and IC₅₀ is determined by fitting a 4-parameter sigmoidal concentration response model.

Each of Examples A1-A868, A983, A1076, A1078-A1081, A1083, A1086, A1091-A1092, A1099-A1102, A1104, A1106-A1110, A1112-A117, A1121-A1135, A1140-A1143, A1154-A1156, A1168, A1174, A1176-A1180, A1187-A1189, A1191, and A1193-A1196 exhibited an IC50 of less than 650 nM with respect to K-RasG12V (Capan-1) in at least one of the assays noted above. Each of Examples A876-A925, A927-A1023, A1025-A1036, A1038-A1081, A1083-A1110, A1112-A1129, and A1131-1196 exhibited an IC50 of less than 750 nM with respect to NCI-H1975.

### Determination of Cell Viability in RAS Mutant Cancer Cell Lines

### Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay is to determine the effects of test compounds on the proliferation of human cancer cell lines (e.g., MIA PaCa-2 KRAS G13C A12 (K-Ras G13C), NCI-H358 (K-Ras G12C), AsPC-1 (K-Ras G12D), and Capan-1 (K-Ras G12V)) over a 5-day treatment period by quantifying the amount of ATP present at endpoint using the CellTiter-Glo@ 2.0 Reagent (Promega). Cells are seeded at 250 cells/well in 40 μL of growth medium in 384-well assay plates and incubated overnight in a humidified atmosphere of 5% CO₂ at 37° C. On the day of the assay, 10 mM stock solutions of test compounds are first diluted into 3 mM solutions with 100% DMSO. Well-mixed compound solutions (15 μL) are transferred to the next wells containing 30 μL of 100% DMSO and repeated until a 9-concentration 3-fold serial dilution is made (starting assay concentration of 10 μM). Test compounds (132.5 nL) are directly dispensed into the assay plates containing cells. Alternatively, test compounds are prepared in 9 point, 3-fold dilutions in DMSO, with a high concentration of 10, 1 or 0.1 mM and on the day of the assay, test compounds (40 nL) are directly dispensed into the assay plates containing cells. The plates are shaken for 15 seconds at 300 rpm, centrifuged, and incubated in a humidified atmosphere of 5% CO₂ at 37° C. for 5 days. On day 5, assay plates and their contents are equilibrated to room temperature for approximately 30 minutes. CellTiter-Glo® 2.0 Reagent (25 μL) is added, and plate contents are mixed for 2 minutes on an orbital shaker before incubation at room temperature for 10 minutes. Luminescence is measured using the PerkinElmer Enspire. Data are normalized by the following: (Sample signal/Avg. DMSO)*100. The data are fit using a four-parameter logistic fit.

Using the conditions above, each of Examples A1-A868 displayed a greater decrease in cell viability with respect to KRAS^{G12V} (Capan-1) versus wild-type KRAS (A375). Each of Examples A876-A1196 displayed a greater decrease in cell viability with respect to Capan-1 and/or NCI-H2009 versus wild-type KRAS (A375).

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

1. A compound having the structure of Formula I′: 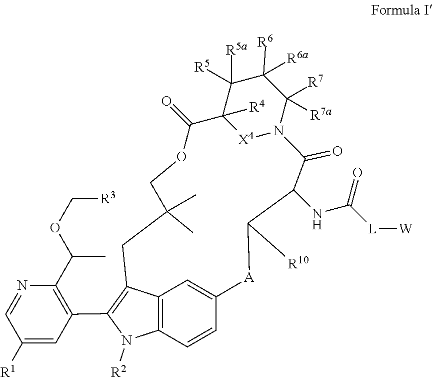
wherein:
A is optionally substituted 3 to 6-membered cycloalkylene, optionally substituted 3- to 10-membered heterocycloalkylene, optionally substituted C₆₋₁₀ arylene, optionally substituted 5 to 6-membered heteroarylene, optionally substituted C₂-C₆ alkylene, or optionally substituted C₂-C₆ alkenylene, or -L¹-L²-, wherein L¹ is 3 to 6-membered cycloalkylene or C₁₋₆ alkylene and L² is —O— or C₁₋₆ alkylene;
L is a bond or —CH(R²⁰)—N(R²¹)—C(O)—, wherein R²⁰ and R²¹ are each independently an optionally substituted C₁₋₆ alkyl; or R²⁰ and R²¹ taken together with the atoms to which they are attached form 4-10 membered heterocycloalkyl;
W is optionally substituted 3 to 10-membered heterocycloalkyl or optionally substituted 3 to 10-membered cycloalkyl;
X⁴ is CH₂ or NH;
R¹ is hydrogen, optionally substituted C₁-C₆ alkyl, optionally substituted C₂₋₆ alkenyl, optionally substituted C₂₋₆ alkynyl, optionally substituted C₁-C₆ heteroalkyl, optionally substituted 3- to 10-membered cycloalkyl, optionally substituted 3- to 6-membered cycloalkenyl, optionally substituted 3- to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6- to 10-membered aryl, optionally substituted 5- to 10-membered heteroaryl;
R³ is hydrogen, optionally substituted C₁-C₆ alkyl, optionally substituted C₁-C₃ heteroalkyl, optionally substituted C₃-C₆ cycloalkyl, or optionally substituted 4- to 6-membered heterocycloalkyl;
or R² and R³ combine, together with the atoms to which they are attached, to form an optionally substituted 8- to 14-membered heterocycloalkyl;
R⁴, R⁵, R⁶, R⁷, R^{5a}, R^{6a} and R^{7a} are each independently hydrogen, halo or C₁-C₆ alkyl;
or R⁵ and R⁷ are taken together to form a methylene or ethylene bridge between the carbon atoms to which they are attached, and R⁴, R^{5a}, R⁶, Roa, and R^{7a} are each independently hydrogen, halogen, or C₁₋₆ alkyl;
or R⁴ and R⁶ are taken together to form a methylene or ethylene bridge between the carbon atoms to which they are attached, and R⁵, R^{5a}, R^{6a}, R⁷ and R^{7a} are each independently hydrogen, halogen, or C₁₋₆ alkyl;
or R⁵ and R⁶ taken together with the atoms to which they are attached form optionally substituted 3- to 7-membered cycloalkyl or optionally substituted 3- to 7-membered heterocycloalkyl;
or R⁶ and R⁷ taken together with the atoms to which they are attached form optionally substituted 3- to 7-membered cycloalkyl or optionally substituted 3- to 7-membered heterocycloalkyl;
or R⁵ and R^{5a} taken together with the atom to which they are attached form optionally substituted 3- to 7-membered cycloalkyl or an optionally substituted 3- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 3- to 7-membered heterocycloalkyl;
or R⁵ and R⁶ taken together form a bond;
R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl;
and
when R¹⁰ is H, then ring A is optionally substituted 2,4-oxazol-diyl or optionally substituted 2,5-oxazol-diyl and R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt thereof.

2. The compound of claim 1, having the structure of Formula I′: 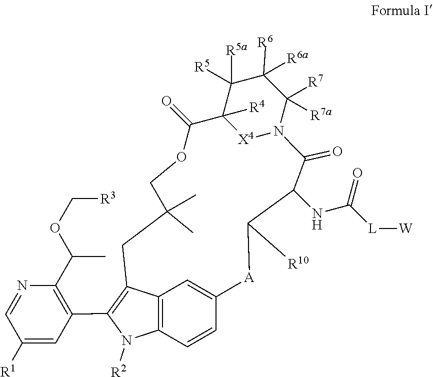
wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
L is a bond or —CH(R²⁰)—N(R²¹)—C(O)—, wherein R²⁰ and R²¹ are each independently an optionally substituted C₁₋₆ alkyl; or R²⁰ and R²¹ taken together with the atoms to which they are attached form 4-10 membered heterocycloalkyl;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R^{5a}, R^{6a} and R^{7a} are each independently hydrogen, halo or C₁-C₆ alkyl;
or R⁵ and R⁶ taken together with the atoms to which they are attached form optionally substituted 3- to 7-membered cycloalkyl or optionally substituted 3- to 7-membered heterocycloalkyl;
or R⁶ and R⁷ taken together with the atoms to which they are attached form optionally substituted 3- to 7-membered cycloalkyl or optionally substituted 3- to 7-membered heterocycloalkyl;
or R⁵ and R^{5a} taken together with the atom to which they are attached form optionally substituted 3- to 7-membered cycloalkyl or an optionally substituted 3- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 3- to 7-membered heterocycloalkyl;
or R⁵ and R⁶ taken together form a bond;
R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl;
and
when R¹⁰ is H, then ring A is optionally substituted 2,4-oxazol-diyl or optionally substituted 2,5-oxazol-diyl and R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt thereof.

3. The compound of claim 1 or 2, wherein the moiety: 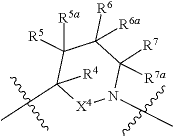
in Formula I′ is selected from: 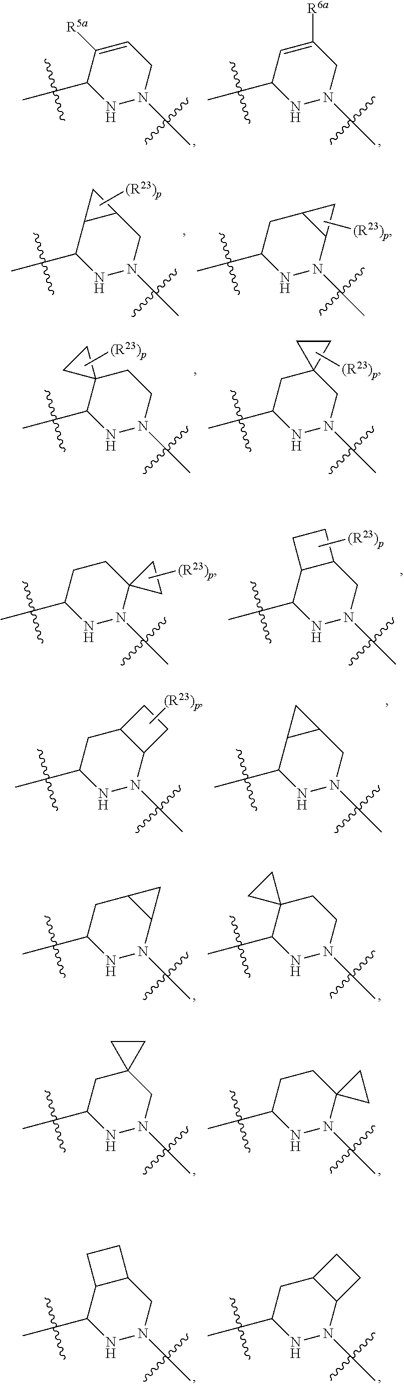 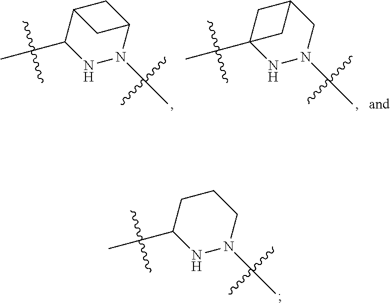
wherein:
R^{5a} is H, halogen or C₁₋₆ alkyl;
R^{6a} is H, halogen or C₁₋₆ alkyl;
each R²³ is independently halogen or C₁₋₆ alkyl; and
each p is independently an integer of 0, 1 or 2.

4. The compound of claim 1 or 2, having the structure of Formula I′-a: 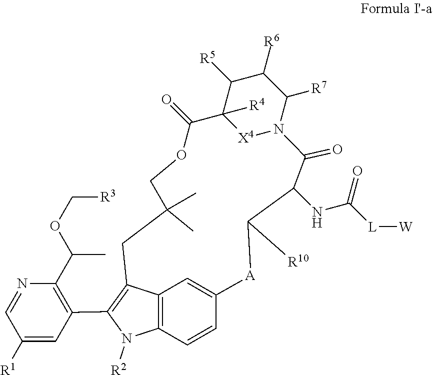
wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
L is a bond or —CH(R²⁰)—N(R²¹)—C(O)—, wherein R²⁰ and R²¹ are each independently an optionally substituted C₁₋₆ alkyl; or R²⁰ and R²¹ taken together with the atoms to which they are attached form 4-10 membered heterocycloalkyl;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R^{5a}, R^{6a} and R^{7a} are each independently hydrogen, halo or C₁-C₆ alkyl;
R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl;
and
when R¹⁰ is H, then ring A is optionally substituted 2,4-oxazol-diyl or optionally substituted 2,5-oxazol-diyl and R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt thereof.

5. The compound of any one of claims 1, 2 and 4 or a pharmaceutically acceptable salt thereof, having the structure of Formula I: 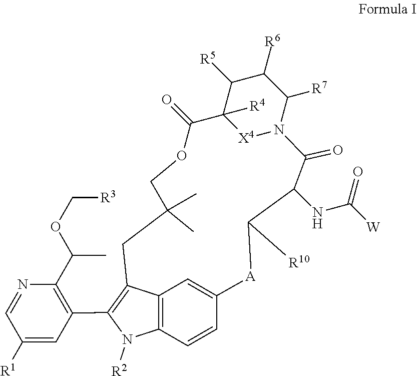
wherein:
A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is H, —OR¹¹ or —NR¹²R¹³;
R¹¹, R¹², and R¹³ are each, independently, optionally substituted C₁-C₆ alkyl, optionally substituted C₁-C₆ heteroalkyl, optionally substituted 4-14 membered heterocycloalkyl, optionally substituted C₃-C₁₀ cycloalkyl or R¹² and R¹³ combine to form an optionally substituted 3- to 10-membered heterocycloalkyl; and
when R¹⁰ is H, then ring A is optionally substituted 2,4-oxazol-diyl or optionally substituted 2,5-oxazol-diyl and R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl;
or a pharmaceutically acceptable salt thereof.

6. The compound of any one of claims 1 to 5 or a pharmaceutically acceptable salt thereof, wherein:
ring A is optionally substituted 2,4-oxazol-diyl or optionally substituted 2,5-oxazol-diyl;
R² is hydrogen, substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, optionally substituted 5 or 6-membered heteroaryl; and
R¹⁰ is H.

7. The compound of any one of claims 1 to 2 and 4 to 6, having the structure of Formula I′-b: 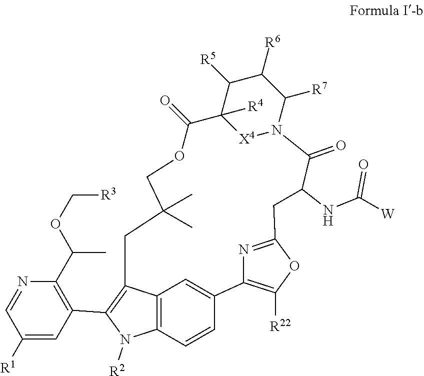
or a pharmaceutically acceptable salt thereof,
wherein R²² is H, CN, OH, optionally substituted C₁₋₆ alkyl, optionally substituted C₁₋₆ alkoxy, optionally substituted C₃₆ cycloalkyl or optionally substituted 4- to 6-membered heterocycloalkyl; and
R² is substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, or optionally substituted 5 or 6-membered heteroaryl.

8. The compound of any one of claims 1 to 7, or a pharmaceutically acceptable salt thereof, wherein R² is optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6-membered aryl, or optionally substituted 5 or 6-membered heteroaryl

9. The compound of any one of claims 1 to 2 and 4 to 8, having the structure of Formula I: 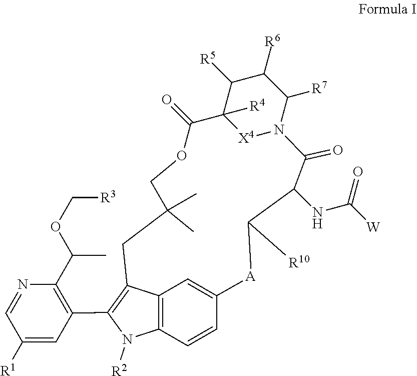
wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
W is optionally substituted 3 to 10-membered heterocycloalkyl or optionally substituted 3 to 10-membered cycloalkyl;
X⁴ is CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R³ is hydrogen;
or R² and R³ combine, together with the atoms to which they are attached, to form an optionally substituted 8- to 14-membered heterocycloalkyl;
each of R⁴, R⁵, R⁶, and R⁷ are hydrogen; or R⁴ and R⁶ are hydrogen and R⁵ and R⁷ combine, together with the atoms to which they are attached, to form an optionally substituted four-membered cycloalkyl; or R⁵ and R⁷ are hydrogen and R⁴ and R⁶ combine, together with the atoms to which they are attached, to form an optionally substituted four-membered cycloalkyl;
R¹⁰ is —OR¹¹ or —NR¹²R¹³; and
R¹¹, R¹², and R¹³ are each, independently, optionally substituted C₁-C₆ alkyl, optionally substituted C₁-C₆ heteroalkyl, or R¹² and R¹³ combine to form an optionally substituted 3- to 10-membered heterocycloalkyl;
or a pharmaceutically acceptable salt thereof,

10. The compound of any one of claims 1 to 9, or a pharmaceutically acceptable salt thereof, wherein X⁴ is NH.

11. The compound of any one of claims 1 to 9, or a pharmaceutically acceptable salt thereof, wherein X⁴ is CH₂.

12. The compound of any one of claims 1 to 9, or a pharmaceutically acceptable salt thereof, having the structure of Formula Ia:

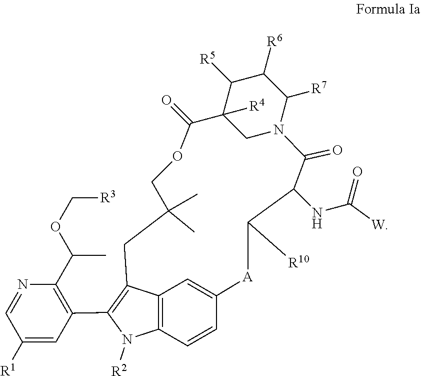

13. The compound of any one of claims 1 to 9, or pharmaceutically acceptable salt thereof, having the structure of Formula Ib:

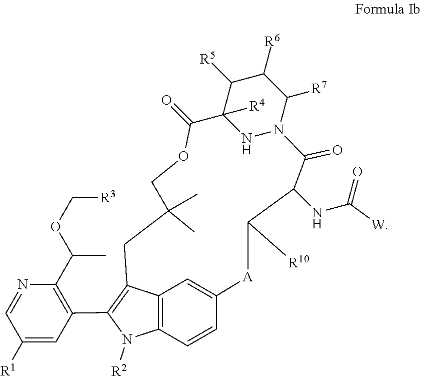

14. The compound of any one of claims 1 to 13, or a pharmaceutically acceptable salt thereof, wherein each of R⁴, R⁵, R⁶, and R⁷ are hydrogen.

15. The compound of any one of claims 1 to 9, or pharmaceutically acceptable salt thereof, having the structure of Formula Ic:

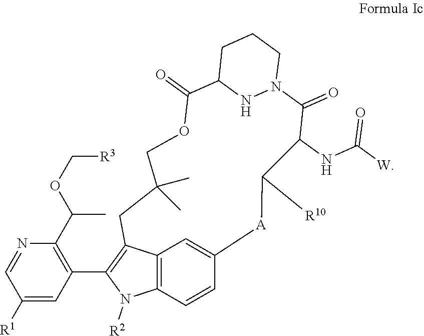

16. The compound of any one of claims 1 to 9, or pharmaceutically acceptable salt thereof, having the structure of Formula Id:

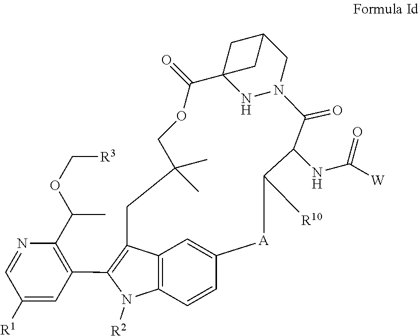

17. The compound of any one of claims 1 to 9, or pharmaceutically acceptable salt thereof, having the structure of Formula Ie:

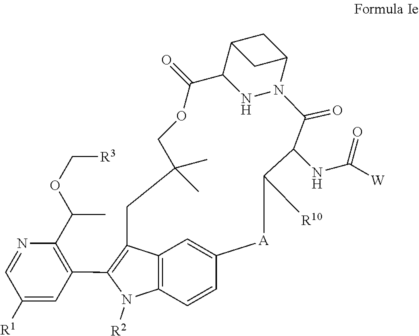

18. The compound of any one of claims 1 to 17, or a pharmaceutically acceptable salt thereof, wherein A is optionally substituted 5 to 6-membered heteroarylene.

19. The compound of claim 18, or a pharmaceutically acceptable salt thereof, wherein A is oxazole-diyl or thiazole-diyl.

20. The compound of any one of claims 1 to 9, or pharmaceutically acceptable salt thereof, having the structure of Formula Ic-1:

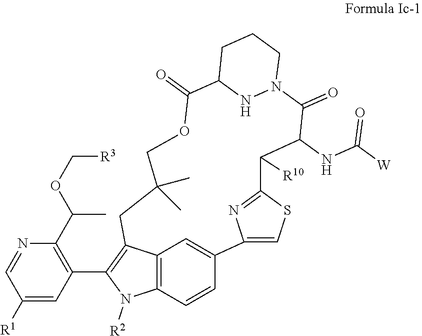

21. The compound of any one of claims 1 to 9, or pharmaceutically acceptable salt thereof, having the structure of Formula Id-1:

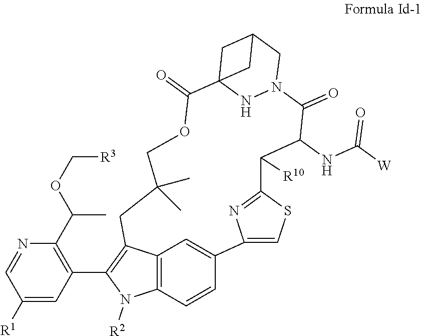

22. The compound of any one of claims 1 to 9, or pharmaceutically acceptable salt thereof, having the structure of Formula Ie-1:

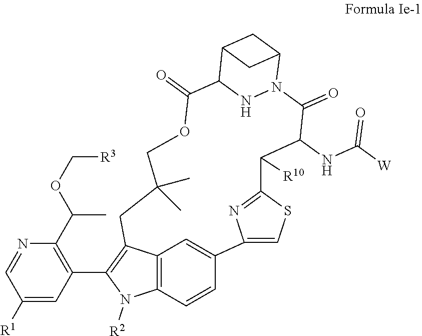

23. The compound of any one of claims 1 to 9, or pharmaceutically acceptable salt thereof, having the structure of Formula Ic-2:

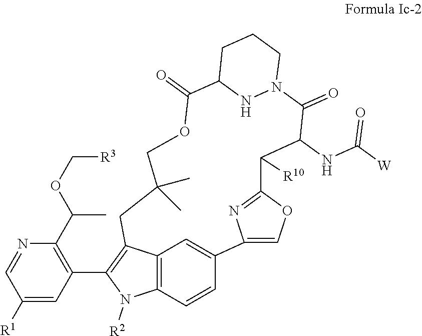

24. The compound of any one of claims 1 to 9, or pharmaceutically acceptable salt thereof, having the structure of Formula Id-2:

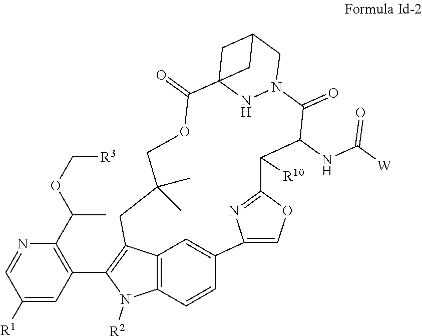

25. The compound of any one of claims 1 to 9, or pharmaceutically acceptable salt thereof, having the structure of Formula Ie-2:

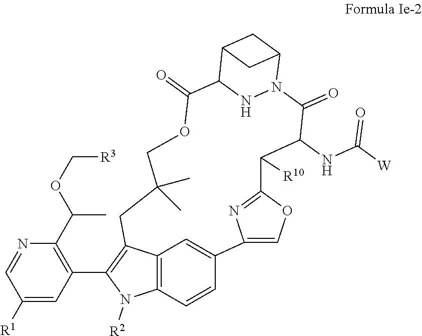

26. The compound of any one of claims 1 to 25, or pharmaceutically acceptable salt thereof, wherein R³ is hydrogen.

27. The compound of any one of claims 1 to 25, or pharmaceutically acceptable salt thereof, wherein R² and R³ combine to form an optionally substituted 8- to 14-membered heterocycloalkyl.

28. The compound of any one of claims 1 to 26, or pharmaceutically acceptable salt thereof, wherein R² is optionally substituted C₁-C₆ heteroalkyl or optionally substituted 3 to 7-membered heterocycloalkyl.

29. The compound of claim 28, or pharmaceutically acceptable salt thereof, wherein R² is optionally substituted C₁-C₆ heteroalkyl.

30. The compound of claim 28, or pharmaceutically acceptable salt thereof, wherein R² is optionally substituted 3 to 7-membered heterocycloalkyl.

31. The compound of any one of claims 1 to 9, or a pharmaceutically acceptable salt thereof, having the structure of Formula Ic-3: 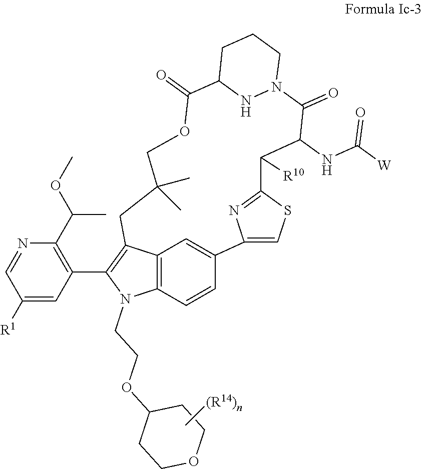
wherein each R¹⁴ is independently C₁-C₃ alkyl; and
n is 0, 1, 2, or 3.

32. The compound of any one of claims 1 to 9, or pharmaceutically acceptable salt thereof, having the structure of Formula Id-3: 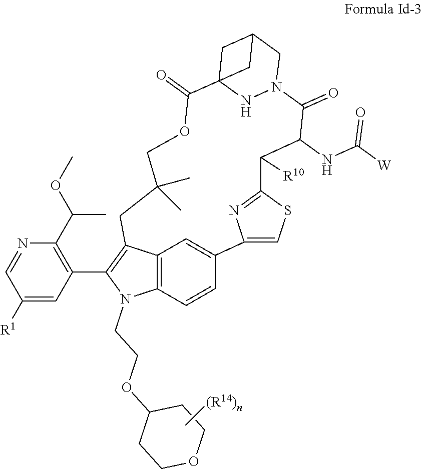
wherein each R¹⁴ is independently C₁-C₃ alkyl; and
n is 0, 1, 2, or 3.

33. The compound of any one of claims 1 to 9, or pharmaceutically acceptable salt thereof, having the structure of Formula Ie-3: 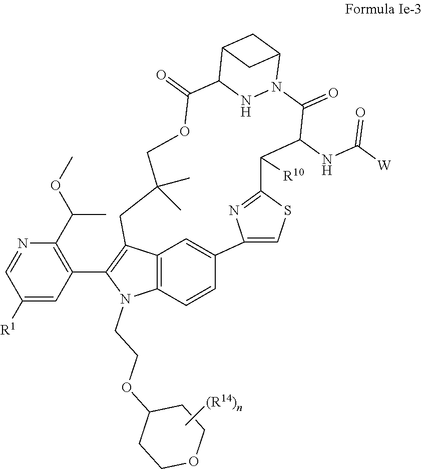
wherein each R¹⁴ is independently C₁-C₃ alkyl; and
n is 0, 1, 2, or 3.

34. The compound of any one of claims 1 to 9, or pharmaceutically acceptable salt thereof, having the structure of Formula Ic-4: 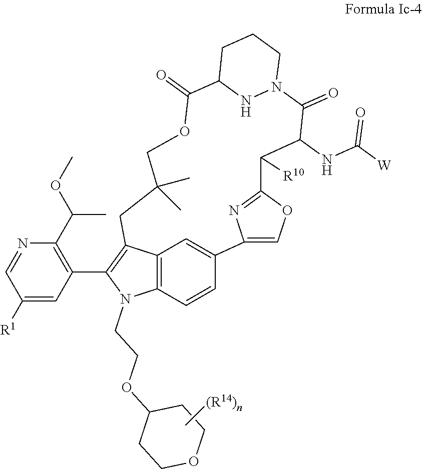
wherein each R¹⁴ is independently C₁-C₃ alkyl; and
n is 0, 1, 2, or 3.

35. The compound of any one of claims 1 to 9, or pharmaceutically acceptable salt thereof, having the structure of Formula Id-4: 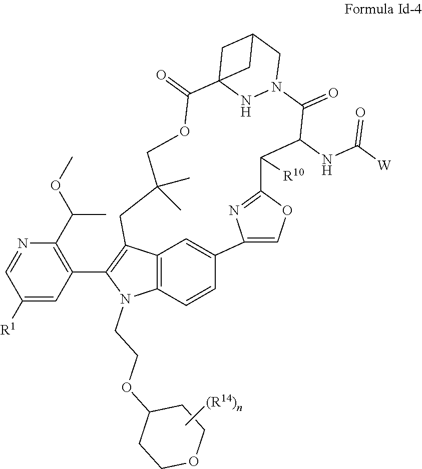
wherein each R¹⁴ is independently C₁-C₃ alkyl; and
n is 0, 1, 2, or 3.

36. The compound of any one of claims 1 to 9, or pharmaceutically acceptable salt thereof, having the structure of Formula Ie-4: 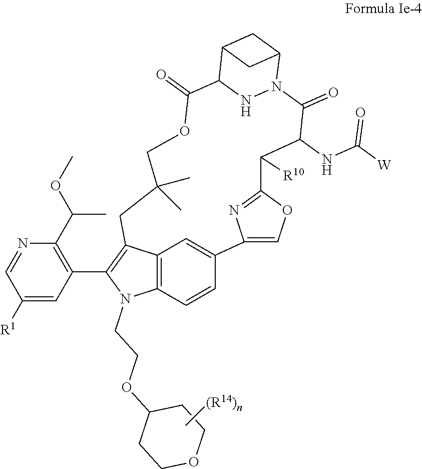
wherein each R¹⁴ is independently C₁-C₃ alkyl; and
n is 0, 1, 2, or 3.

37. The compound of any one of claims 31 to 36, or pharmaceutically acceptable salt thereof, wherein each R¹⁴ is methyl.

38. The compound of any one of claims 31 to 37, or pharmaceutically acceptable salt thereof, wherein n is 1.

39. The compound of any one of claims 31 to 37, or pharmaceutically acceptable salt thereof, wherein n is 2.

40. The compound of any one of claims 31 to 36, or pharmaceutically acceptable salt thereof, wherein n is 0.

41. The compound of any one of claims 1 to 9, or a pharmaceutically acceptable salt thereof, having the structure of Formula Ic-5: 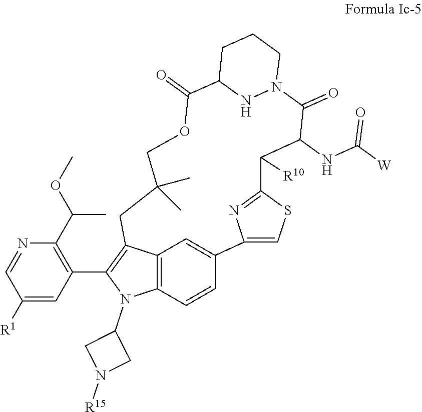
wherein R¹⁶ is C₁-C₃ alkyl or C₃-C₆cycloalkyl.

42. The compound of any one of claims 1 to 9, or pharmaceutically acceptable salt thereof, having the structure of Formula Id-5: 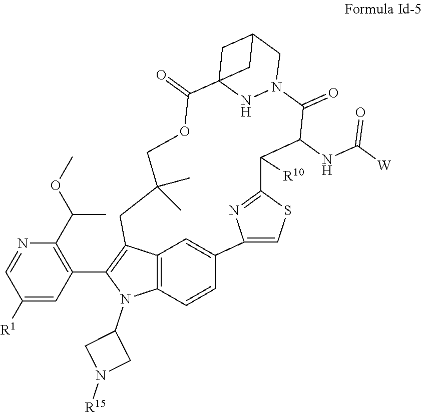
wherein R¹⁵ is C₁-C₃ alkyl or C₃-C₆cycloalkyl.

43. The compound of any one of claims 1 to 9, or pharmaceutically acceptable salt thereof, having the structure of Formula Ie-5: 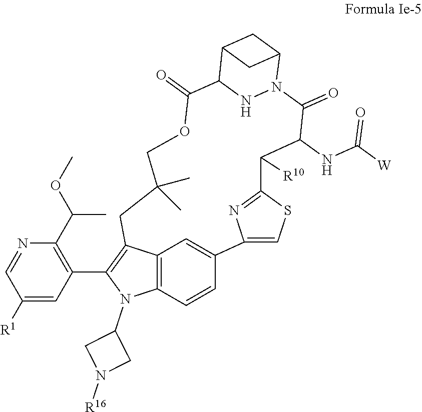
wherein R¹⁵ is C₁-C₃ alkyl or C₃-C₆cycloalkyl.

44. The compound of any one of claims 1 to 9, or pharmaceutically acceptable salt thereof, having the structure of Formula Ic-6: 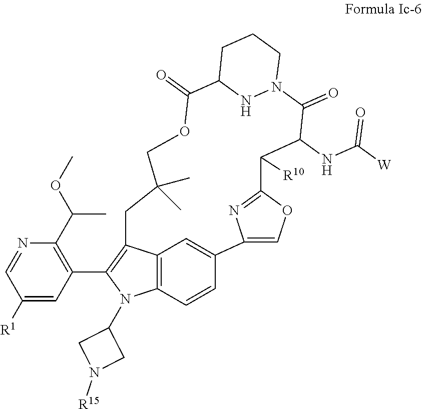
wherein R¹⁵ is C₁-C₃ alkyl or C₃-C₆cycloalkyl.

45. The compound of any one of claims 1 to 9, or pharmaceutically acceptable salt thereof, having the structure of Formula Id-6: 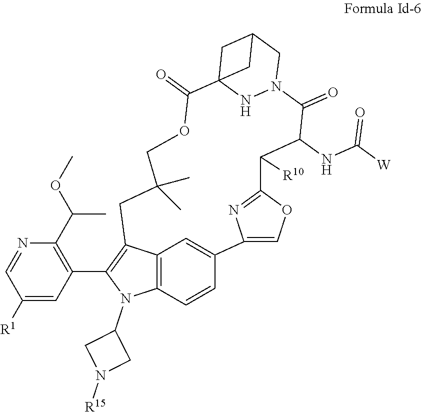
wherein R¹⁶ is C₁-C₃ alkyl or C₃-C₆cycloalkyl.

46. The compound of any one of claims 1 to 9, or pharmaceutically acceptable salt thereof, having the structure of Formula Ie-6: 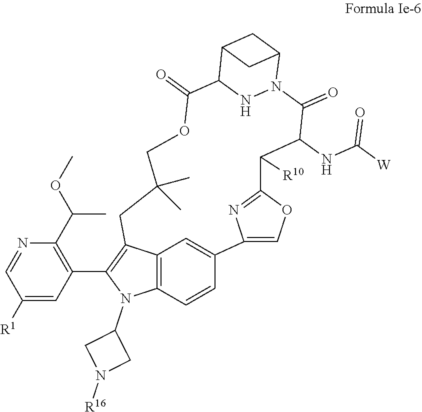
wherein R¹⁵ is C₁-C₃ alkyl or C₃-C₆cycloalkyl.

47. The compound of any one of claims 41 to 46, or a pharmaceutically acceptable salt thereof, wherein R¹⁵ is C₃-C₆ cycloalkyl.

48. The compound of claim 47, wherein R¹⁵ is cyclopropyl.

49. The compound of any one of claims 1 to 48, or a pharmaceutically acceptable salt thereof, wherein R¹ is hydrogen.

50. The compound of any one of claims 1 to 48, or a pharmaceutically acceptable salt thereof, wherein R¹ is 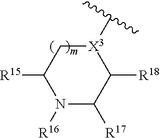
wherein X³ is N or CH;
m is 1 or 2;
R¹⁵, R¹⁶, R¹⁷, and R¹⁸ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R¹⁶ and R¹⁷ combine with the atoms to which they are attached to form an optionally substituted 3 to 8-membered heterocycloalkyl; or
R¹⁷ and R¹⁸ combine with the atoms to which they are attached to form an optionally substituted 3 to 8-membered heterocycloalkyl; or
R¹⁷ and R¹⁵ combine with the atoms to which they are attached to form an optionally substituted 4 to 8-membered heterocycloalkyl.

51. The compound of claim 50, or a pharmaceutically acceptable salt thereof, wherein R¹ is

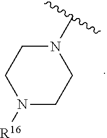

52. The compound of claim 50, or a pharmaceutically acceptable salt thereof, wherein R¹ is

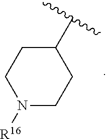

53. The compound of any one of claims 50 to 52, or a pharmaceutically acceptable salt thereof, wherein R¹⁶ is methyl.

54. The compound of any one of claims 50 to 52, or a pharmaceutically acceptable salt thereof, wherein R¹⁶ is cyclopropyl.

55. The compound of any one of claims 1 to 54, or a pharmaceutically acceptable salt thereof, wherein W is optionally substituted 3 to 10-membered heterocycloalkyl.

56. The compound of claim 55, or a pharmaceutically acceptable salt thereof, wherein W is optionally substituted 4- to 6-membered optionally substituted heterocycloalkyl.

57. The compound of claim 55 or 56, or a pharmaceutically acceptable salt thereof, wherein the heterocycloalkyl comprises one oxygen atom.

58. The compound of claim 55 or 56, or a pharmaceutically acceptable salt thereof, wherein the heterocycloalkyl comprises one nitrogen atom.

59. The compound of any one of claims 1 to 54, or a pharmaceutically acceptable salt thereof, wherein W is optionally substituted 3 to 10-membered heterocycloalkyl.

60. The compound of claim 59, or a pharmaceutically acceptable salt thereof, wherein W is optionally substituted 3- to 6-membered optionally substituted heterocycloalkyl

61. The compound of claim 60, or a pharmaceutically acceptable salt thereof, wherein W is optionally substituted cyclopropyl.

62. The compound of any one of claims 1 to 61, or a pharmaceutically acceptable salt thereof, wherein R¹⁰ is —OR¹¹.

63. The compound of claim 62, or a pharmaceutically acceptable salt thereof, wherein R¹¹ is optionally substituted C₁-C₆ alkyl.

64. The compound of claim 62, or a pharmaceutically acceptable salt thereof, wherein R¹¹ is optionally substituted C₁-C₆ heteroalkyl.

65. The compound of claim 62, or a pharmaceutically acceptable salt thereof, wherein R¹⁰ is —OCH₂CH₃.

66. The compound of any one of claims 1 to 61, or a pharmaceutically acceptable salt thereof, wherein R¹⁰ is —NR¹²R¹³.

67. The compound of claim 66, or a pharmaceutically acceptable salt thereof, wherein R¹² and R¹³ combine to form an optionally substituted 3- to 10-membered heterocycloalkyl.

68. The compound of claim 67, or a pharmaceutically acceptable salt thereof, wherein R¹² and R¹³ combine to form an optionally substituted pyrrolidine.

69. The compound of claim 67, or a pharmaceutically acceptable salt thereof, wherein R¹² and R¹³ combine to form an optionally substituted azetidine.

70. The compound of claim any one of claims 1 to 61, or a pharmaceutically acceptable salt thereof, wherein R¹⁰ is 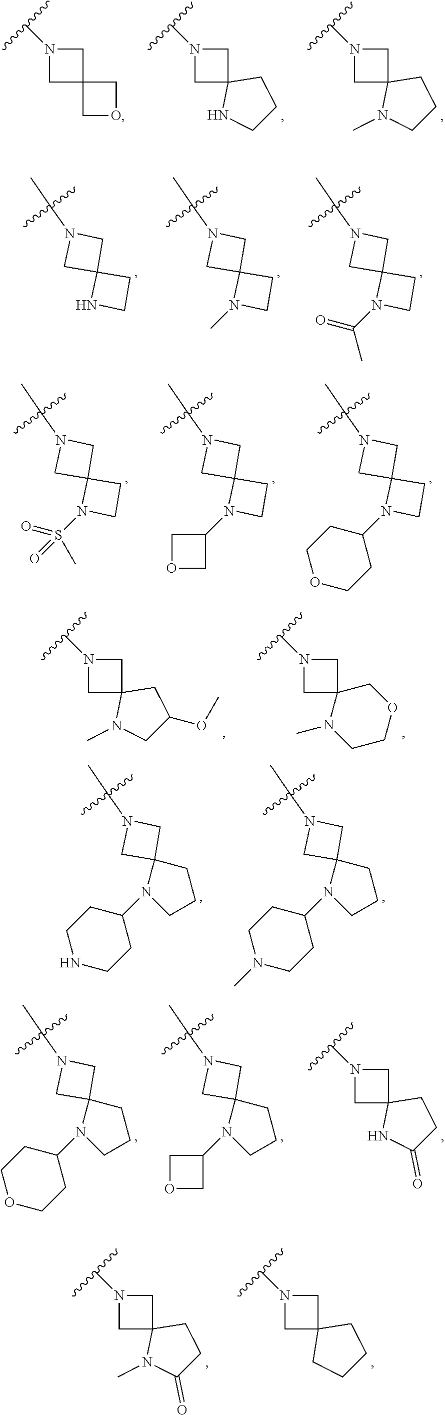 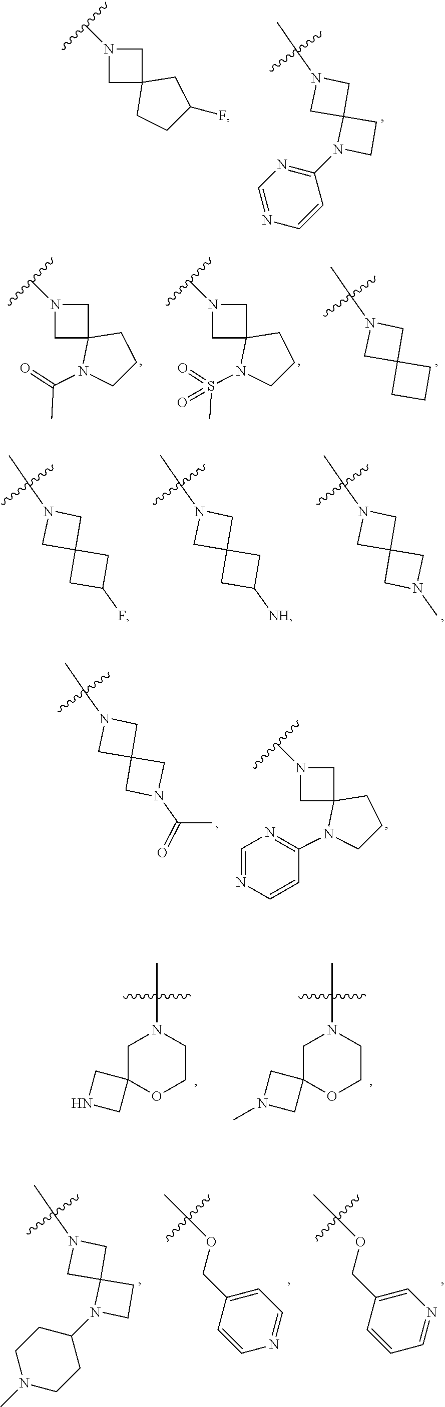 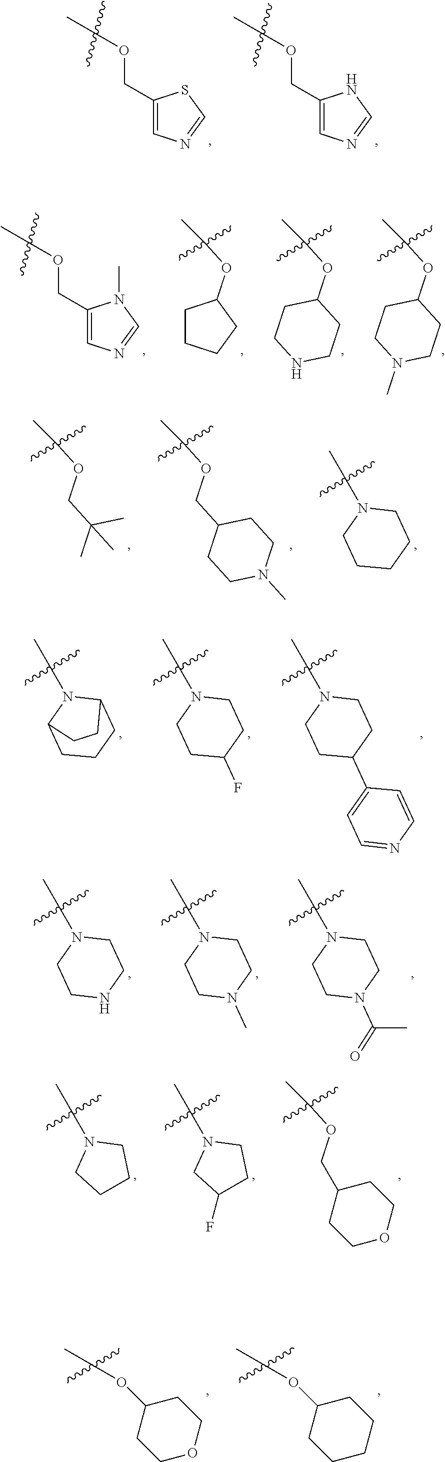 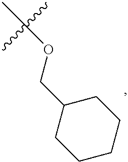 [SMILES: *OCC1CCCCC1]
—OCH₃, —N(CH₃)₂, —OCH₂CH(CH₃)₂, or —OCH₂SO₂CH₃, each of which is optionally substituted.

71. A compound of Table 1, or a pharmaceutically acceptable salt thereof.

72. A compound of Table 2, or a pharmaceutically acceptable salt thereof.

73. A pharmaceutical composition comprising a compound, or a pharmaceutically acceptable salt thereof, of any one of claims 1 to 72 and a pharmaceutically acceptable excipient

74. A method of treating cancer in a subject in need thereof, the method comprising administering to the subject a therapeutically effective amount of a compound, or a pharmaceutically acceptable salt thereof, of any one of claims 1 to 72 or a pharmaceutical composition of claim 73.

75. The method of claim 74, wherein the cancer is pancreatic cancer, colorectal cancer, non-small cell lung cancer, gastric cancer, esophageal cancer, ovarian cancer or uterine cancer.

76. The method of claim 75, wherein the cancer comprises a Ras mutation.

77. The method of claim 76, wherein the Ras mutation is K-Ras G12V.

78. A method of treating a Ras protein-related disorder in a subject in need thereof, the method comprising administering to the subject a therapeutically effective amount of a compound, or a pharmaceutically acceptable salt thereof, of any one of claims 1 to 72 or a pharmaceutical composition of claim 73.

79. The method of any one of claims 74 to 78, wherein the method further comprises administering to the subject an additional anticancer agent or therapy.

80. The method of claim 79, wherein the additional anticancer agent or therapy is an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, a HER2 inhibitor, or a combination thereof.

81. A compound of Formula V: 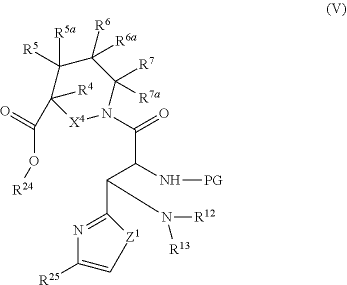
wherein:
R⁴, R⁵, R⁶, R⁷, R^{5a}, R^{6a} and R^{7a} are each independently hydrogen, halo or C₁-C₆ alkyl;
or R⁵ and R⁷ are taken together to form a methylene or ethylene bridge between the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form a methylene or ethylene bridge between the carbon atoms to which they are attached, and R⁵, R^{5a}, R^{6a}, R⁷ and R^{7a} are each independently hydrogen, halogen, or C₁₋₆ alkyl;
or R⁵ and R⁶ taken together with the atoms to which they are attached form optionally substituted 3- to 7-membered cycloalkyl or optionally substituted 3- to 7-membered heterocycloalkyl;
or R⁶ and R⁷ taken together with the atoms to which they are attached form optionally substituted 3- to 7-membered cycloalkyl or optionally substituted 3- to 7-membered heterocycloalkyl;
or R⁵ and R^{5a} taken together with the atom to which they are attached form optionally substituted 3- to 7-membered cycloalkyl or an optionally substituted 3- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 3- to 7-membered heterocycloalkyl;
or R⁵ and R⁶ taken together form a bond;
X⁴ is NH or CH₂;
Z¹ is O or S;
PG is an amino protecting group;
R²⁴ is H or C₁₋₆ alkyl;
R²⁵ is halogen; and
R¹², and R¹³ are each, independently, optionally substituted C₁-C₆ alkyl, optionally substituted C₁-C₆ heteroalkyl, or R¹² and R¹³ taken together with the nitrogen atom to which they are attached form optionally substituted 3- to 11-membered heterocycloalkyl.

82. The compound of claim 81, wherein X⁴ is NH.

83. The compound of claim 81 or 82, wherein R²⁴ is C₁₋₆ alkyl.

84. The compound of any one of claims 81 to 83, wherein PG is Boc or Cbz.

85. The compound of any one of claims 81 to 84, wherein R²⁵ is Br or I.

86. The compound of any one of claims 81 to 85, wherein R¹² and R¹³ taken together with the nitrogen atom to which they are attached form optionally substituted 7- to 11-membered sprio heterocycloalkyl.

87. The compound of any one of claims 81 to 86, wherein R⁵ and R⁷ are taken together to form a methylene bridge between the carbon atoms to which they are attached, and R⁴, R^{5a}, R⁶, R^{6a}, and R^{7a} are each independently hydrogen, halogen, or C₁₋₆ alkyl.

88. The compound of any one of claims 81 to 87, wherein the moiety: 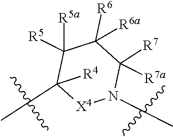
in Formula (V) is 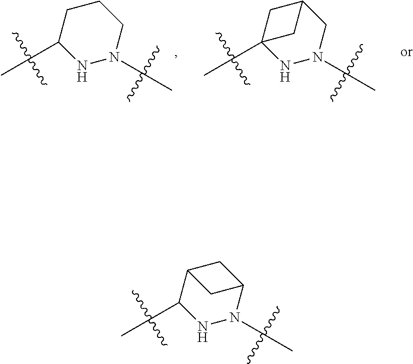 [SMILES: *C1CCCN(*)N1 ; *N1CC2CC(*)(C2)N1 ; *C1NN(*)C2CC1C2]
R⁵ and R⁷ are taken together to form a methylene or ethylene bridge between the carbon atoms to which they are attached, and R⁴, R⁵, R^{5a}, R⁶, R^{6a}, R⁷ and R^{7a} are each independently hydrogen, halogen, or C₁₋₆ alkyl.

89. The compound of any one of claims 81 to 88, wherein Z¹ is O.

90. The compound of any one of claims 81 to 88, wherein Z¹ is S.

91. A compound of Formula (VI): 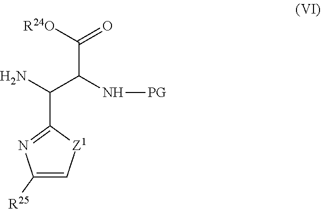
wherein:
Z¹ is O or S;
PG is an amino protecting group;
R²⁴ is H or C₁₋₆ alkyl; and
R²⁵ is halogen.

92. The compound of claim 91, wherein Z¹ is O.

93. The compound of claim 91, wherein Z¹ is S.

94. The compound of any one of claims 91 to 93, wherein PG is Boc or Cbz.

95. The compound of any one of claims 91 to 94, wherein R²⁴ is C₁₋₆ alkyl.

96. The compound of any one of claims 91 to 95, wherein R²⁵ is Br or I.

97. The compound of claim 91, wherein Z¹ is O, R²⁵ is I, PG is Boc and R¹⁴ is Et.

98. A compound of Formula VII: 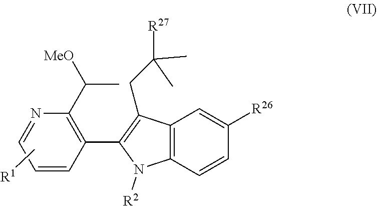
wherein:
R¹ is hydrogen, optionally substituted C₁-C₆ alkyl, optionally substituted C₂₋₆ alkenyl, optionally substituted C₂₋₆ alkynyl, optionally substituted C₁-C₆ heteroalkyl, optionally substituted 3- to 10-membered cycloalkyl, optionally substituted 3- to 6-membered cycloalkenyl, optionally substituted 3- to 15-membered heterocycloalkyl, optionally substituted 6 to 10-membered aryl, optionally substituted 5 to 10-membered heteroaryl, —B(OH)₂ or 4,4,5,5-tetramethyl-1,3,2-dioxaborolan-2-yl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6- to 10-membered aryl, optionally substituted 5- to 10-membered heteroaryl;
R²⁶ is halogen, toluenesulfonyloxy, CH₃SO₃—, CF₃SO₃— or —B(OH)₂; and
R²⁷ is —CH₂—OH, —CH₂O-PG¹, —COOH or —COOR²⁸;
R²⁸ is C₁₋₆ alkyl or NR²⁹R²⁹;
R²⁹ is H or independently C₁₋₆ alkyl; and
PG¹ is a hydroxy protecting group.

99. The compound of claim 98, wherein R¹ is H, 4-methylpiperazin-1-yl, 4-cyclopropylpiperazin-1-yl, —B(OH)₂ or 4,4,5,5-tetramethyl-1,3,2-dioxaborolan-2-yl.

100. The compound of claim 98 or 99, wherein R² is H, C₁₋₆ alkyl, 2-((tetrahydro-2H-pyran-4-yl)oxy)ethyl, or 3-cyanocyclobutyl.

101. The compound of any one of claims 98 to 100, wherein R²⁶ is Br or I.

102. The compound of any one of claims 98 to 101, wherein PG¹ is TBDPS.

103. The compound of any one of claims 98 to 102, wherein R²⁷ is —CH₂OH.

104. The compound of claim 98, wherein the compound is selected from:

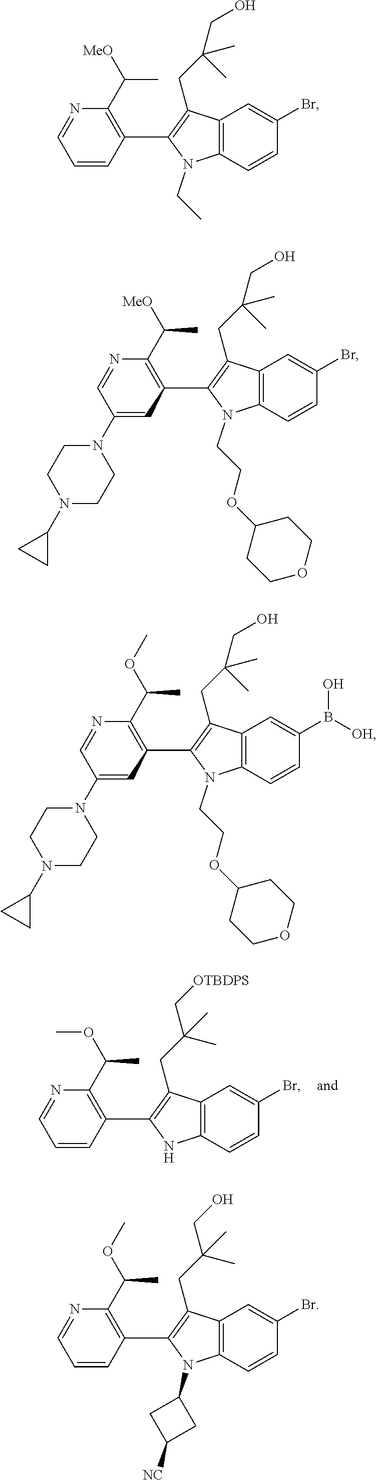

105. A compound of Formula (VIII): 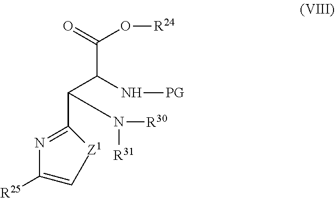
wherein:
Z¹ is O or S;
PG is H or an amino protecting group;
R³⁰ and R³¹ are each H; or R³⁰ and R³¹ taken together with the nitrogen atom to which they are attached form optionally substituted 7- to 11-membered spiro heterocycloalkyl;
R²⁴ is H or C₁₋₆ alkyl; and
R²⁵ is Br or I.

106. The compound of claim 105, wherein R²⁴ is H.

107. The compound of claim 105 or 106, wherein PG is Boc.

108. The compound of any one of claims 105 to 107, wherein R³⁰ and R³¹ are each H.

109. The compound of any one of claims 105 to 107, wherein R³⁰ and R³¹ taken together with the nitrogen atom to which they are attached form optionally substituted 7- to 11-membered spiro heterocycloalkyl.

110. The compound of any one of claims 105 to 109, wherein Z¹ is O.

111. The compound of any one of claims 105 to 109, wherein Z¹ is S.

112. The compound of claim 105, wherein the compound is:

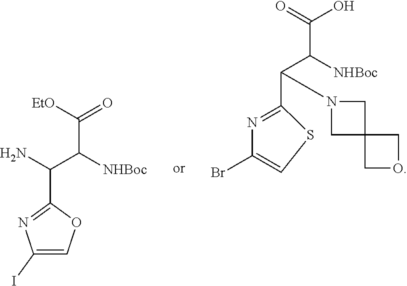 [SMILES: CCOC(=O)C(NC(=O)OC(C)(C)C)C(N)c1nc(I)co1 ; CC(C)(C)OC(=O)NC(C(=O)O)C(c1nc(Br)cs1)N1CC2(COC2)C1]

113. A compound of Formula (IX): 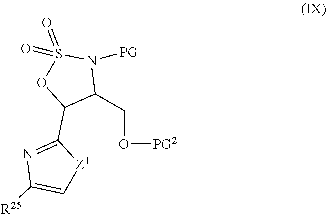
wherein:
Z¹ is O or S;
PG is an amino protecting group;
PG² is a hydroxy protecting group; and
R²⁵ is Br or I.

114. The compound of claim 113, wherein PG is Boc.

115. The compound of claim 113 or 114, wherein PG² is —C(O) CHs.

116. The compound of any one of claims 113 to 115, wherein Z¹ is O.

117. The compound of any one of claims 113 to 115, wherein Z¹ is S.

118. The compound of any one of claims 113 to 117, wherein R²⁵ is Br.

119. A compound of Formula (X): 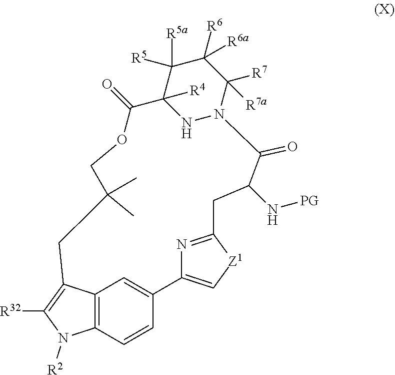
wherein:
PG is H or an amino protecting group;
Z¹ is O or S;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6- to 10-membered aryl, optionally substituted 5- to 10-membered heteroaryl;
R³² is halogen, —B(OH)₂ or 4,4,5,5-tetramethyl-1,3,2-dioxaborolan-2-yl;
R⁴, R⁵, R⁶, R⁷, R^{5a}, R^{6a} and R^{7a} are each independently hydrogen, halo or C₁-C₆ alkyl;
or R⁵ and R⁷ are taken together to form a methylene or ethylene bridge between the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form a methylene or ethylene bridge between the carbon atoms to which they are attached, and R⁵, R^{5a}, R^{6a}, R⁷ and R^{7a} are each independently hydrogen, halogen, or C₁₋₆ alkyl;
or R⁵ and R⁶ taken together with the atoms to which they are attached form optionally substituted 3- to 7-membered cycloalkyl or optionally substituted 4- to 7-membered heterocycloalkyl;
or R⁶ and R⁷ taken together with the atoms to which they are attached form optionally substituted 3- to 7-membered cycloalkyl or optionally substituted 4- to 7-membered heterocycloalkyl;
or R⁵ and R^{5a} taken together with the atom to which they are attached form optionally substituted 3- to 7-membered cycloalkyl or an optionally substituted 4- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 4- to 7-membered heterocycloalkyl;
or R⁵ and R⁶ taken together form a bond.

120. The compound of claim 119, wherein PG is Boc.

121. The compound of claim 119 or 120, wherein R² is H or optionally substituted C₁₋₆ alkyl.

122. The compound of any one of claims 119 to 121, wherein R³² is 4,4,5,5-tetramethyl-1,3,2-dioxaborolan-2-yl, Br or I.

123. The compound of any one of claims 119 to 122, wherein Z¹ is O.

124. The compound of any one of claims 119 to 122, wherein Z¹ is S.

125. The compound of any one of claims 119 to 124, wherein R⁴, R⁵, R⁶, R⁷, R^{5a}, R^{6a} and R^{7a} are each hydrogen.

126. The compound of any one of claims 119 to 124, wherein R⁵ and R⁷ are taken together to form a methylene bridge between the carbon atoms to which they are attached, and R⁴, R^{5a}, R⁶, R^{6a}, and R^{7a} are each hydrogen.

127. A compound of Formula (XI): 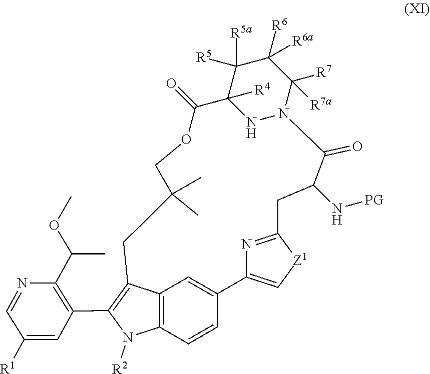
wherein:
PG is H or an amino protecting group;
Z¹ is O or S;
R¹ is hydrogen, optionally substituted C₁-C₆ alkyl, optionally substituted C₂₋₆ alkenyl, optionally substituted C₂₋₆ alkynyl, optionally substituted C₁-C₆ heteroalkyl, optionally substituted 3- to 10-membered cycloalkyl, optionally substituted 3- to 6-membered cycloalkenyl, optionally substituted 3- to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₃-C₁₀ cycloalkyl, optionally substituted 4 to 10-membered heterocycloalkyl, optionally substituted 6- to 10-membered aryl, optionally substituted 5- to 10-membered heteroaryl;
R⁴, R⁵, R⁶, R⁷, R^{5a}, R^{6a} and R^{7a} are each independently hydrogen, halo or C₁-C₆ alkyl;
or R⁵ and R⁷ are taken together to form a methylene or ethylene bridge between the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form a methylene or ethylene bridge between the carbon atoms to which they are attached, and R⁵, R^{5a}, R^{6a}, R⁷ and R^{7a} are each independently hydrogen, halogen, or C₁₋₆ alkyl;
or R⁵ and R⁶ taken together with the atoms to which they are attached form optionally substituted 3- to 7-membered cycloalkyl or optionally substituted 4- to 7-membered heterocycloalkyl;
or R⁶ and R⁷ taken together with the atoms to which they are attached form optionally substituted 3- to 7-membered cycloalkyl or optionally substituted 4- to 7-membered heterocycloalkyl;
or R⁵ and R^{5a} taken together with the atom to which they are attached form optionally substituted 3- to 7-membered cycloalkyl or an optionally substituted 4- to 7-membered heterocycloalkyl;
or R⁶ and R^{6a} taken together with the atom to which they are attached form optionally substituted cycloalkyl or optionally substituted heterocycloalkyl;
or R⁷ and R^{7a} taken together with the atom to which they are attached form an optionally 3- to 7-membered substituted cycloalkyl or an optionally substituted 4- to 7-membered heterocycloalkyl;
or R⁵ and R⁶ taken together form a bond.

128. The compound of claim 127, wherein PG is H or Boc.

129. The compound of claim 127 or 128, wherein R¹ is H or optionally substituted heterocycloalkyl.

130. The compound of any one of claims 127 to 129, wherein R² is H or optionally substituted C₁₋₆ alkyl.

131. The compound of any one of claims 127 to 130, wherein Z¹ is O.

132. The compound of any one of claims 127 to 130, wherein Z¹ is S.

133. The compound of any one of claims 127 to 132, wherein R⁴, R⁵, R⁶, R⁷, R^{5a}, R^{6a} and R^{7a} are each hydrogen.

134. The compound of any one of claims 128 to 132, wherein R⁵ and R⁷ are taken together to form a methylene bridge between the carbon atoms to which they are attached, and R⁴, R^{5a}, R⁶, R^{6a}, and R^{7a} are each hydrogen.
